(19) United States
(12) Patent Application Publication
(10) Pub. No.: US 2026/0200920 A1
(43) Pub. Date: Jul. 16, 2026

(54) SUBSTITUTED AMINOPYRIDINE COMPOUNDS FOR USE AS AKT1 INHIBITORS

(71) Applicant: Atavistik Bio, Inc., Cambridge, MA (US)

(72) Inventors: Maria Jesus Blanco-Pillado, Arlington, MA (US); Aaron Robert Coffin, Everett, MA (US); Anil Kumar Padyana, Cambridge, MA (US)

(21) Appl. No.: 19/561,295

(22) Filed: Mar. 9, 2026

Related U.S. Application Data
(63) Continuation of application No. PCT/US2024/046772, filed on Sep. 13, 2024.
(60) Provisional application No. 63/538,712, filed on Sep. 15, 2023.
(60) Provisional application No. 63/568,711, filed on Mar. 22, 2024.

(51) Int. Cl. C07D 471/04; A61K 31/4439; A61K 31/444; A61K 31/4545; A61K 31/5377; C07D 401/04; C07D 401/14; C07D 405/14

(52) U.S. Cl. CPC C07D 471/04; A61K 31/4439; A61K 31/444; A61K 31/4545; A61K 31/5377; C07D 401/04; C07D 401/14; C07D 405/14

(57) ABSTRACT

The invention provides aminopyridinyl imidazo[4,5-b]pyridine of formula (I) and related compounds, pharmaceutical compositions, and their use in treating disease, such as cancer.

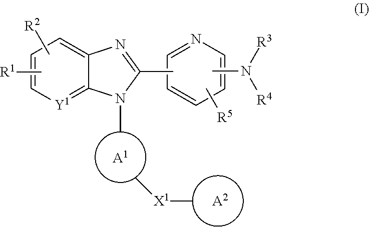

### CROSS-REFERENCE TO RELATED APPLICATIONS

This application claims priority to and the benefit of U.S. Provisional Patent Application Nos 63/538,712, filed Sep. 15, 2023, and 63/568,711, filed Mar. 22, 2024, the contents of each of which are hereby incorporated by reference in their entirety.

### BRIEF SUMMARY

The invention provides aminopyridinyl imidazo[4,5-b]pyridine and related compounds, pharmaceutical compositions, and their use in treating disease, such as cancer BACKGROUND

Cancer continues to be a significant health problem despite the substantial research efforts and scientific advances reported in the literature for treating this disease. Solid tumors, such as prostate cancer, colon, rectum, skin cancer, breast cancer, and lung cancer remain highly prevalent among the world population. Existing therapies for treating cancer include localized therapies, such as surgery, radiation therapy, cryotherapy, and systemic therapies (e.g., chemotherapy, hormonal therapy, immune therapy, and targeted therapy) used alone or in combination. Support therapies are also used in some contexts, where supportive therapies are additional treatments that do not directly treat cancer but are used to reduce side effects and address patient quality of life. However, current treatment options for cancer are not effective for all patients and/or can have substantial adverse side effects. New therapies are needed to address this unmet need in cancer therapy.

The AKT protein family, which members are also named as protein kinase B (PKB), are important mediators of growth factor induced cellular signaling. See, for example, Manning and Toker, Cell 169, Apr. 20, 2017. The AKT protein family belongs to the larger serine/threonine kinase family regulated by the phosphoinositide 3-kinase (PI3K) pathway—a critical signal transduction system linking oncogenes and multiple receptor classes to many essential cellular functions. Class I PI3K phosphorylates the 3′ hydroxyl of the inositol head group of phosphoinositides (PtdIns), resulting in the production of the lipid second messengers PtdIns-3,4-P₂ (PIP2) and PtdIns3,4,5-P3 (PIP3). PIP3 recruits AKT to the plasma membrane where it is activated and triggers a signaling cascade impinging upon pathways important for cancer cell growth.

In humans, there are three AKT isoforms encoded by three separate genes: AKT1, AKT2 and AKT3. These genes encode for serine/threonine kinases that are activated by growth factors in a phosphoinositide 3-kinase (PI3K) dependent manner. AKT1 regulates cell growth and survival and is expressed in several tissues. AKT2 is an important signaling molecule in the insulin signaling pathway and is required to induce glucose transport in certain tissues. The role of AKT3 is less known, and it appears to be predominantly expressed in the brain.

Among the three AKT isoforms, AKT1 is most frequently hyperactivated in tumors. The hyperactivation of AKT1 in tumors underscores the important role AKT1 plays in tumorigenesis and cancer cell proliferation and survival (Bhattarai et al. Nature Communications volume 13, Article number: 2111, 2022). AKT1 signaling promotes tumor cell growth by modulating multiple different effectors. For example, AKT1 regulates protein synthesis via the mTORC1 pathway, cellular metabolism via GSK3-beta pathway and multiple metabolic enzymes, and cell proliferation and survival via the FOXO family of transcription factors. See, for example, Hoxhaj and Manning, Nat Rev Cancer, 2020 February; 20(2): 74-88.

The most common mutation in AKT1 is a point mutation converting a glutamic acid of residue 17 to a lysine (E17K). The E17K mutation [Carpten et al. Nature 448, 2007, 439-444] in the Pleckstrin Homology Domain (PHD) of AKT1 leads to constitutive association with the plasma membrane via ectopic binding to PIP2 and increased affinity for PIP3. Consequently, this deregulated recruitment of AKT1 to the plasma membrane causes constitutive activation of AKT1 signaling and has been shown to be a recurrent mutation in breast, endometrial, and other cancers.

The oncogenic properties of the E17K single point mutation in AKT1 make it a target for inhibition for anti-cancer agents. In addition to cancer, somatic mutation of AKT1 to the E17K variant during embryonic development leads to the overgrowth disease Proteus Syndrome (N Engl J Med. 2011 Aug. 18; 365(7): 611-619). Proteus syndrome is characterized by the overgrowth of skin, connective tissue, brain, and other tissues. Patients with this disease also have an increased risk of premature death due to vascular deformities.

New compounds that inhibit AKT1 are needed and would provide a medical benefit to patients suffering from disorders associated with AKT1 activity. The present invention addresses the foregoing needs and provides other related advantages.

### SUMMARY

The invention provides aminopyridinyl imidazo[4,5-b]pyridine and related compounds, pharmaceutical compositions, and their use in treating disease, such as cancer. In particular, one aspect of the invention provides a collection of aminopyridinyl imidazo[4,5-b]pyridine and related compounds, such as a compound represented by Formula I:

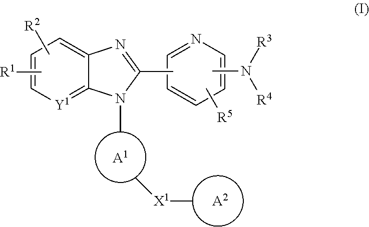

or a stereoisomer or pharmaceutically acceptable salt thereof, where the variables are as defined in the detailed description. Further description of additional collections of aminopyridinyl imidazo[4,5-b]pyridine and related compounds are described in the detailed description. The compounds may be part of a pharmaceutical composition comprising a pharmaceutically acceptable carrier.

Another aspect of the invention provides a method of treating a disease or disorder associated with aberrant AKT1 signaling. The method comprises administering to a subject in need thereof a therapeutically effective amount of a compound described herein, such as a compound of Formula I, to treat the disease or disorder, as further described in the detailed description.

Another aspect of the invention provides a method of treating cancer. The method comprises administering to a subject in need thereof a therapeutically effective amount of a compound described herein, such as a compound of Formula I, to treat the cancer, as further described in the detailed description. In certain embodiments, the cancer has a AKT1 mutation.

A method of treating a disease or disorder associated with active PI3K signaling. The method comprises administering to a subject in need thereof a therapeutically effective amount of a compound described herein, such as a compound of Formula I, to treat the disease or disorder.

Another aspect of the invention provides a method inhibiting AKT1 activity. The method comprises contacting an AKT1 with an effective amount of a compound described herein, such as a compound of Formula I, to thereby inhibit the AKT1 activity, as further described in the detailed description.

### DETAILED DESCRIPTION

The invention provides aminopyridinyl imidazo[4,5-b]pyridine and related compounds, pharmaceutical compositions, and their use in treating disease, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito, 1999, and “March's Advanced Organic Chemistry”, 5^{th} Ed, Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In certain embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In certain embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In certain embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

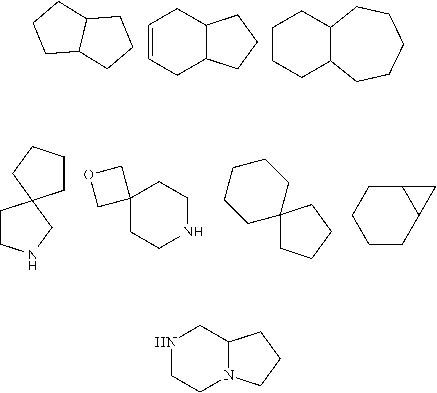

Exemplary bridged bicyclics include:

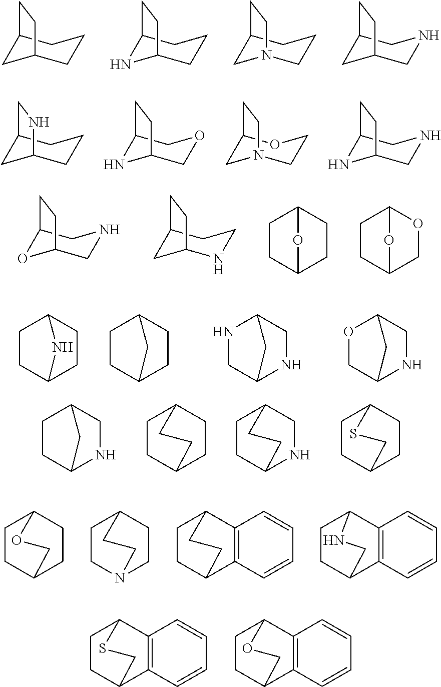

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group (e.g., a bivalent branched alkylene group or a bivalent linear alkylene chain group). An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” and “halo” are used interchangeably and mean F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it

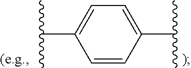 [SMILES: *c1ccc(*)cc1]

“phenylene” is a trivalent phenyl group when it has three groups attached to it

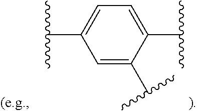 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by one or more oxo group. The term “heterocyclylene” refers to a multivalent heterocyclyl group having the appropriate number of open valences to account for groups attached to it. For example, “heterocyclylene” is a bivalent heterocyclyl group when it has two groups attached to it, “heterocyclylene” is a trivalent heterocyclyl group when it has three groups attached to it. The term “oxo-heterocyclylene” refers to a multivalent oxo-heterocyclyl group having the appropriate number of open valences to account for groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}, —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R⁴; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}2; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —C(O)R^{∘}₂; —OP(O)R^{∘}₂; —OP(O)(OR^{∘})₂; SiR^{∘}₃; —(C₁₋₄ a straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}. —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, J=NNHC(O)R*, J=NNHC(O)OR*, J=NNHS(O)₂R*, J=NR*, J=NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}, wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2 naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Cami Ile G. (eds.) Handbook of Pharmaceutical Salts, Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York, and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, the replacement of a carbon by a ¹³C- or ¹⁴C-enriched carbon, or the replacement of a fluorine by a ¹⁸F-enriched fluorine are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Heterosubstituted Aminopyridinyl Imidazo[4,5-b]pyridine & Related Compounds

One aspect of the invention provides aminopyridinyl imidazo[4,5-b]pyridine and related compounds. The compounds may be used in the pharmaceutical compositions and therapeutic methods described herein. Exemplary compounds are described in the following sections, along with exemplary procedures for making the compounds.

One aspect of the invention provides a compound represented by Formula I

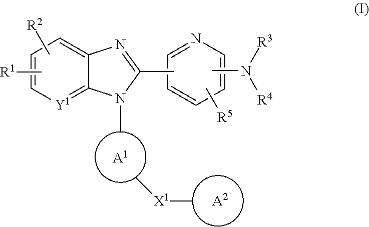

or a stereoisomer or pharmaceutically acceptable salt thereof; wherein:
R¹ is phenyl, a C₁₋₆ monocyclic cycloalkyl, a 5-6 membered heteroaryl containing 1 or 2 heteroatoms selected from nitrogen, a 5-6 membered saturated heterocyclyl containing 1 or 2 heteroatoms independently selected from nitrogen, oxygen, and sulfur, C₁₋₄ alkoxy or C₁₋₆ alkyl, wherein the phenyl, cycloalkyl, heteroaryl, heterocyclyl, and alkoxy are each substituted with m occurrences of R¹²;
R², R³, R⁴, R⁵, and R⁹ are each independently selected from hydrogen, C₁₋₄ alkyl, and C₁₋₄ haloalkyl;
R⁶ is halo or C₁₋₄ alkyl;
R⁷ is —(C₀₋₄ alkylene)-CO₂H, —CO₂(C₁₋₄ alkyl), S(O)₂N(R⁹)₂ or —C(O)N(R⁹)₂;
R⁸ represents independently for each occurrence C₁₋₄ alkyl, C₁₋₄ haloalkyl, or halo;
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R¹² represents independently for each occurrence halo, cyano, C₁₋₄ alkoxy or C₁₋₄ alkyl;
A¹ is phenylene or pyridinylene, each of which is substituted with 0, 1, or 2 occurrences of R⁶;
A² is a C₄₋₆ monocyclic cycloalkyl, C₅₋₈ bridged or fused bicyclic cycloalkyl, dioxo-cyclohexyl, C₇₋₁₀ spirocyclic saturated carbocyclyl, 7-10 membered spirocyclic saturated heterocyclyl containing 1 heteroatom selected from nitrogen or a 5-6 membered saturated heterocyclyl containing 1 or 2 heteroatoms independently selected from nitrogen, oxygen, and sulfur, wherein the cycloalkyl, carbocyclyl, and heterocyclyl are substituted with (i) 1 occurrence of R⁷ and (ii) n occurrences of R⁸;
X¹ is —N(R⁹)C(O)-Ψ, —C(O)N(R⁹)-Ψ, (3-6 membered saturated heterocyclylene containing 1 or 2 heteroatoms selected from nitrogen)-C(O)-Ψ, —N(R⁹)C(R¹⁰)(R¹¹)-Ψ, —N(R⁹)-Ψ, —C(O)-Ψ, —(C(R¹⁰)(R¹¹))ₚ-Ψ, -(3-6 membered saturated heterocyclylene containing 1 or 2 heteroatoms selected from nitrogen)-C(R¹⁰)(R¹¹)-Ψ, —(C(R¹⁰)(R¹¹))ₚ—N(R⁹)-Ψ, or a covalent bond; wherein Ψ is a bond to A², and wherein the 3-6 membered saturated heterocyclylene is substituted with 0, 1, or 2 occurrences of C₁₋₄ alkyl;
Y¹ is N or —C(H)—;
p is 1, 2, 3, 4, 5, or 6 and
m and n are independently 0, 1, 2, 3, or 4,
   wherein when X¹ is a covalent bond, A² is not cyclobutyl.

In certain embodiments, the compound is represented by Formula I:

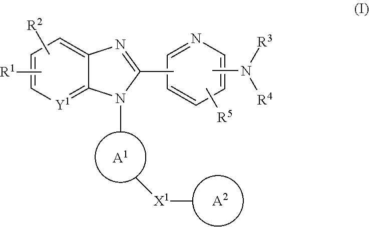

or a stereoisomer or pharmaceutically acceptable salt thereof; wherein:
R¹ is phenyl, a C₃₋₆ monocyclic cycloalkyl, a 5-6 membered heteroaryl containing 1 or 2 heteroatoms selected from nitrogen, a 5-6 membered saturated heterocyclyl containing 1 or 2 heteroatoms independently selected from nitrogen, oxygen, and sulfur, C₁₋₄ alkoxy or C₁₋₆ alkyl, wherein the phenyl, cycloalkyl, heteroaryl, heterocyclyl, and alkoxy are each substituted with m occurrences of R¹²;
R², R³, R⁴, R⁵, and R⁹ are independently hydrogen or C₁₋₄ alkyl;
R⁶ is halo or C₁₋₄ alkyl;
R⁷ is —(C₀₋₄ alkylene)-CO₂H, —CO₂(C₁₋₄ alkyl), or —C(O)NH₂;
R⁸ represents independently for each occurrence C₁₋₄ alkyl, C₁₋₄ haloalkyl, or halo;
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring:
R¹² represents independently for each occurrence halo, cyano, or C₁₋₄ alkyl;
A¹ is phenylene or pyridinylene, each of which is substituted with 0, 1, or 2 occurrences of R⁶;
A² is a C₄₋₆ monocycliccycloalkyl, C₃₋₇ bridged or fused bicyclic cycloalkyl, dioxo-cyclohexyl, C₇₋₁₀ spirocyclic saturated carbocyclyl, 7-10 membered spirocyclic saturated heterocyclyl containing 1 heteroatom selected from nitrogen or a 5-6 membered saturated heterocyclyl containing 1 or 2 heteroatoms independently selected from nitrogen, oxygen, and sulfur, wherein the cycloalkyl, carbocyclyl, and heterocyclyl are substituted with (i) 1 occurrence of R⁷ and (ii) n occurrences of R⁸;
X¹ is —N(R⁹)C(O)-Ψ, —C(O)N(R⁹)-Ψ, —(C₁₋₃ alkylene)-N(R⁹)C(O)-Ψ, -(3-5 membered saturated heterocyclylene containing 1 or 2 heteroatoms selected from nitrogen)-C(O)-Ψ, —N(R⁹)C(R¹⁰)(R¹¹)-Ψ, —N(R⁹)-Ψ, —C(O)-Ψ, —C(R¹⁰)(R¹¹)-Ψ, -(3-6 membered saturated heterocyclylene containing 1 or 2 heteroatoms selected from nitrogen)-C(R¹⁰)(R¹¹)-Ψ, —C(R¹⁰)(R¹¹)—N(R⁹)-Ψ, or a covalent bond; wherein Ψ is a bond to A²;
Y¹ is N or —C(H)—; and
m and n are independently 0, 1, 2, or 3,
wherein when X¹ is a covalent bond, A² is not cyclobutyl.

In another aspect, the invention provides a compound represented by Formula I:

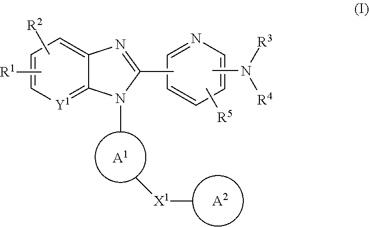

or a stereoisomer or pharmaceutically acceptable salt thereof; wherein:
R¹ is phenyl, a C₃₋₆ monocyclic cycloalkyl, a 5-6 membered heteroaryl containing 1 or 2 heteroatoms selected from nitrogen, a 5-6 membered saturated heterocyclyl containing 1 or 2 heteroatoms independently selected from nitrogen, oxygen, and sulfur, C₁₋₄ alkoxy or C₁₋₆ alkyl, wherein the phenyl, cycloalkyl, heteroaryl, heterocyclyl, and alkoxy are each substituted with m occurrences of R¹²
R², R³, R⁴, R⁵, and R⁹ are independently hydrogen or C₁₋₄ alkyl;
R⁶ is halo or C₁₋₄ alkyl;
R⁷ is —(C₀₋₄ alkylene)-CO₂H, —CO₂(C₁₋₄ alkyl), or —C(O)NH₂;
R⁸ represents independently for each occurrence C₁₋₄ alkyl, C₁₋₄ haloalkyl, or halo;
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring,
R¹² represents independently for each occurrence halo, cyano, or C₁₋₄ alkyl:
A¹ is phenylene or pyridinylene, each of which is substituted with 0, 1, or 2 occurrences of R⁶;
A² is a C₅₋₆ monocycliccycloalkyl, C₅₋₇ bridged or fused bicyclic cycloalkyl, dioxo-cyclohexyl, C₇₋₁₀ spirocyclic saturated carbocyclyl, 7-10 membered spirocyclic saturated heterocyclyl containing 1 heteroatom selected from nitrogen or a 5-6 membered saturated heterocyclyl containing 1 or 2 heteroatoms independently selected from nitrogen, oxygen, and sulfur, wherein the cycloalkyl, carbocyclyl, and heterocyclyl are substituted with (i) 1 occurrence of R⁷ and (ii) n occurrences of R⁸;
X¹ is —N(R⁹)C(O)-Ψ, —C(O)N(R⁹)-Ψ, —(C₁₋₃ alkylene)-N(R⁹)C(O)-Ψ, -(3-5 membered saturated heterocyclylene containing 1 or 2 heteroatoms selected from nitrogen)-C(O)-Ψ, —N(R⁹)C(R¹⁰)(R¹¹)-Ψ, —N(R⁹)-Ψ, —C(O)-Ψ, —C(R¹⁰)(R¹¹)-Ψ, -(3-6 membered saturated heterocyclylene containing 1 or 2 heteroatoms selected from nitrogen)-C(R¹⁰)(R¹¹)-Ψ, —C(R¹⁰)(R¹¹)—N(R⁹)-Ψ, or a covalent bond; wherein Ψ is a bond to A²;
Y¹ is N or —C(H)—; and
m and n are independently 0, 1, 2, or 3.

In another aspect, the invention provides a compound represented by Formula I:

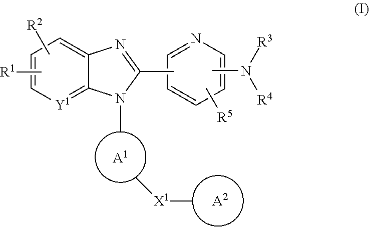

or a stereoisomer or pharmaceutically acceptable salt thereof; wherein:
R¹ is phenyl, a 5-6 membered heteroaryl containing 1 or 2 heteroatoms selected from nitrogen, a 5-6 membered saturated heterocyclyl containing 1 or 2 heteroatoms independently selected from nitrogen, oxygen, and sulfur, or C₁₋₆ alkyl, wherein the phenyl, heteroaryl, and heterocyclyl are each substituted with m occurrences of R¹²;
R², R³, R⁴, R⁵, and R⁹ are independently hydrogen or C₁₋₄ alkyl;
R⁶ is halo or C₁₋₄ alkyl;
R⁷ is —(C₀₋₄ alkylene)-CO₂H;
R⁸ represents independently for each occurrence C₁₋₄ alkyl, C₁₋₄ haloalkyl, or halo;
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R¹² represents independently for each occurrence halo, cyano, or C₁₋₄ alkyl;
A¹ is phenylene or pyridinylene, each of which is substituted with 0 or 1 occurrence of R⁶;
A² is a C₄₋₆ monocyclic cycloalkyl, C₅₋₇ bridged or fused bicyclic cycloalkyl, dioxo-cyclohexyl, C₇₋₁₀ spirocyclic saturated carbocyclyl, or a 5-6 membered saturated heterocyclyl containing 1 or 2 heteroatoms independently selected from nitrogen, oxygen, and sulfur, wherein the cycloalkyl, carbocyclyl, and heterocyclyl are substituted with (i) 1 occurrence of R⁷ and (ii) n occurrences of R⁸;
X¹ is —N(R⁹)C(O)-Ψ, —C(O)N(R⁹)-Ψ, —(C₁₋₃ alkylene)-N(R⁹)C(O)-Ψ, -(3-5 membered saturated heterocyclylene containing 1 or 2 heteroatoms selected from nitrogen)-C(O)-Ψ, —N(R⁹)C(R¹⁰)(R¹¹)-Ψ, —N(R⁹)—V, —C(O)-Ψ, —C(R¹⁰)(R¹¹)-Ψ, -(3-5 membered saturated heterocyclylene containing 1 or 2 heteroatoms selected from nitrogen)-C(R¹⁰)(R¹¹)-Ψ, —C(R¹⁰)(R¹¹)—N(R⁹)-Ψ, or a covalent bond; wherein T is a bond to A²;
Y¹ is N or —C(H)—; and
m and n are independently 0, 1, or 2.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As generally defined above, R¹ is phenyl, a C₃₋₆ monocyclic cycloalkyl, a 5-6 membered heteroaryl containing 1 or 2 heteroatoms selected from nitrogen, a 5-6 membered saturated heterocyclyl containing 1 or 2 heteroatoms independently selected from nitrogen, oxygen, and sulfur, C₁₋₄ alkoxy or C₁₋₆ alkyl, wherein the phenyl, cycloalkyl, heteroaryl, heterocyclyl, and alkoxy are each substituted with m occurrences of R².

In certain embodiments, R¹ is phenyl, a 5-6 membered heteroaryl containing 1 or 2 heteroatoms selected from nitrogen, a 5-6 membered saturated heterocyclyl containing 1 or 2 heteroatoms independently selected from nitrogen, oxygen, and sulfur, or C₁₋₆ alkyl, wherein the phenyl, heteroaryl, and heterocyclyl are each substituted with m occurrences of R².

In certain embodiments, R¹ is phenyl substituted with m occurrences of R¹². In certain embodiments, R¹ is a 6-membered heteroaryl containing 1 or 2 heteroatoms selected from nitrogen substituted with m occurrences of R¹². In certain embodiments, R¹ is a 5-membered heteroaryl containing 1 or 2 heteroatoms selected from nitrogen substituted with m occurrences of R¹². In certain embodiments, R¹ is a 5-6 membered saturated heterocyclyl containing 1 or 2 heteroatoms independently selected from nitrogen, oxygen, and sulfur substituted with m occurrences of R². In certain embodiments, R¹ is phenyl. In certain embodiments, R¹ is C₁₋₆ alkyl. In certain embodiments, R¹ is selected from the groups depicted in the compounds in Table 1, below.

As generally defined above, R², R³, R⁴, R⁵, and R⁹ are each independently selected from hydrogen, C₁₋₄ alkyl, and C₁₋₄ haloalkyl. In certain embodiments, R², R³, R⁴, R⁵, and R⁹ are independently hydrogen or C₁₋₄ alkyl. In certain embodiments, R² is hydrogen or C₁₋₄ alkyl. In certain embodiments, R³ is hydrogen or C₁₋₄ alkyl. In certain embodiments, R⁴ is hydrogen or C₁₋₄ alkyl. In certain embodiments, R⁵ is hydrogen or C₁₋₄ alkyl. In certain embodiments, R⁹ is hydrogen or C₁₋₄ alkyl. In certain embodiments, R² is C₁₋₄ alkyl. In certain embodiments, R³ is C₁₋₄ alkyl. In certain embodiments, R⁴ is C₁₋₄ alkyl. In certain embodiments, R⁵ is C₁₋₄ alkyl. In certain embodiments, R⁹ is C₁₋₄ alkyl. In certain embodiments, R⁹ is C₁₋₄ haloalkyl. In certain embodiments, R², R³, R⁴, R⁵, and R⁹ are hydrogen. In certain embodiments, R², R³, R⁴, R⁵ and R⁹ are C₁₋₄ alkyl.

In certain embodiments, R¹ is a C₃₋₆ monocyclic cycloalkyl substituted with m occurrences of R¹². In certain embodiments, R¹ is a cyclopropyl substituted with m occurrences of R¹². In certain embodiments, R¹ is a cyclobutyl substituted with m occurrences of R¹². In certain embodiments, R¹ is a cyclopentyl substituted with m occurrences of R¹². In certain embodiments, R¹ is a cyclohexyl substituted with m occurrences of R¹².

In certain embodiments, R¹ is a C₁₋₄ alkoxy substituted with m occurrences of R². In certain embodiments, R¹ is a methoxy substituted with m occurrences of R². In certain embodiments, R¹ is an ethoxy substituted with m occurrences of R². In certain embodiments, R¹ is a propoxy substituted with m occurrences of R². In certain embodiments, R¹ is a butoxy substituted with m occurrences of R¹².

In certain embodiments, R² is hydrogen. In certain embodiments, R³ is hydrogen. In certain embodiments, R⁴ is hydrogen. In certain embodiments, R⁵ is hydrogen. In certain embodiments, R⁹ is hydrogen.

In certain embodiments, R² is selected from the groups depicted in the compounds in Table 1, below. In certain embodiments, R³ is selected from the groups depicted in the compounds in Table 1, below. In certain embodiments, R⁴ is selected from the groups depicted in the compounds in Table 1, below. In certain embodiments, R⁵ is selected from the groups depicted in the compounds in Table 1, below. In certain embodiments, R⁹ is selected from the groups depicted in the compounds in Table 1, below.

As generally defined above, R⁶ is halo or C₁₋₄ alkyl. In certain embodiments. R⁶ is halo. In certain embodiments, R⁶ is C₁₋₄ alkyl. In certain embodiments, R⁶ is selected from the groups depicted in the compounds in Table 1, below.

As generally defined above, R⁷ is —(C₀₋₄ alkylene)-CO₂H, —CO₂(C₁₋₄ alkyl), S(O)₂N(R⁹)₂ or —C(O)N(R⁹)₂. In certain embodiments, R⁷ is —(C₀₋₄ alkylene)-CO₂H, —CO₂(C₁₋₄ alkyl) or —C(O)NH₂. In certain embodiments, R⁷ is —CO₂CH₃. In certain embodiments, R⁷ is —C(O)NH₂. In certain embodiments, R⁷ is S(O)₂N(R⁹)₂. In certain embodiments, R⁷ is —C(O)N(R⁹)₂. In certain embodiments, R⁷ is S(O)₂NHCH₃. In certain embodiments, R⁷ is C(O)NHCH₃.

In certain embodiments, R⁷ is —(C₁₋₄ alkylene)-CO₂H. In certain embodiments, R⁷ is —CO₂H. In certain embodiments, R⁷ is —(C₁₋₃ alkylene)-CO₂H. In certain embodiments, R⁷ is —CH₂CO₂H. In certain embodiments, R¹ is —C(H)(CH₃)CO₂H. In certain embodiments, R⁷ is selected from the groups depicted in the compounds in Table 1, below.

As generally defined above, R⁸ represents independently for each occurrence C₁₋₄ alkyl, C₁₋₄ haloalkyl, or halo. In certain embodiments, R⁸ represents independently for each occurrence C₁₋₄ alkyl. In certain embodiments, R⁸ represents independently for each occurrence C₁₋₄ haloalkyl. In certain embodiments, R⁸ represents independently for each occurrence halo. In certain embodiments, R⁸ is methyl. In certain embodiments, R⁸ is selected from the groups depicted in the compounds in Table 1, below.

As generally defined above, R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring. In certain embodiments, R¹⁰ and R¹¹ are hydrogen. In certain embodiments, R¹⁰ and R¹¹ C₁₋₄ alkyl. In certain embodiments, R¹⁰ is hydrogen. In certain embodiments, R¹¹ is hydrogen. In certain embodiments, R¹⁰ is C₁₋₄ alkyl. In certain embodiments, R¹¹ is C₁₋₄ alkyl. In certain embodiments, R¹⁰ and R¹¹ are taken together with the nitrogen atom to which they are attached to form a 3-7 membered ring. In certain embodiments, R¹⁰ is selected from the groups depicted in the compounds in Table 1, below. In certain embodiments, R¹¹ is selected from the groups depicted in the compounds in Table 1, below.

As generally defined above, R¹² represents independently for each occurrence halo, cyano, C₁₋₄ alkoxy or C₁₋₄ alkyl. In certain embodiments, R¹² represents independently for each occurrence halo, cyano, or C₁₋₄ alkyl. In certain embodiments, R¹² represents independently for each occurrence halo. In certain embodiments, R¹² represents independently for each occurrence C₁₋₄ alkyl. In certain embodiments, R¹² represents independently for each occurrence C₁₋₄ alkoxy. In certain embodiments, R¹² is cyano. In certain embodiments, R¹² is selected from the groups depicted in the compounds in Table 1, below.

As generally defined above, A¹ is phenylene or pyridinylene, each of which is substituted with 0, 1, or 2 occurrences of R⁶. In certain embodiments, A¹ is phenylene or pyridinylene, each of which is substituted with 0 or 1 occurrence of R⁶. In certain embodiments, A¹ is phenylene substituted with 0, 1, or 2 occurrences of R⁶. In certain embodiments, A¹ is phenylene substituted with 0 or 1 occurrence of R⁶. In certain embodiments, A¹ is phenylene substituted with 1 occurrence of R⁶. In certain embodiments, A¹ is phenylene substituted with 2 occurrences of R⁶. In certain embodiments, A¹ is pyridinylene substituted with 0, 1, or 2 occurrences of R⁶. In certain embodiments, A¹ is pyridinylene substituted with 0 or 1 occurrence of R⁶. In certain embodiments, A¹ is pyridinylene substituted with 1 occurrence of R⁶. In certain embodiments, A¹ is pyridinylene substituted with 2 occurrences of R⁶. In certain embodiments, A¹ is phenylene. In certain embodiments, A¹ is pyridinylene. In certain embodiments, A¹ is selected from the groups depicted in the compounds in Table 1, below.

As generally defined above, A² is a C₄₋₆ monocyclic cycloalkyl, C₅₋₈ bridged or fused bicyclic cycloalkyl, dioxo-cyclohexyl, C₇₋₁₀ spirocyclic saturated carbocyclyl, 7-10 membered spirocyclic saturated heterocyclyl containing 1 heteroatom selected from nitrogen or a 5-6 membered saturated heterocyclyl containing 1 or 2 heteroatoms independently selected from nitrogen, oxygen, and sulfur, wherein the cycloalkyl, carbocyclyl, and heterocyclyl are substituted with (i) 1 occurrence of R⁷ and (ii) n occurrences of R⁸. In certain embodiments, A² is a C₄₋₆ monocyclic cycloalkyl, C₅₋₇ bridged or fused bicyclic cycloalkyl, dioxo-cyclohexyl, C₇₋₁₀ spirocyclic saturated carbocyclyl, 7-10 membered spirocyclic saturated heterocyclyl containing 1 heteroatom selected from nitrogen or a 5-6 membered saturated heterocyclyl containing 1 or 2 heteroatoms independently selected from nitrogen, oxygen, and sulfur, wherein the cycloalkyl, carbocyclyl, and heterocyclyl are substituted with (i) 1 occurrence of R⁷ and (ii) n occurrences of R⁸.

In certain embodiments, A² is a C₅₋₆ monocyclic cycloalkyl, C₅₋₇ bridged or fused bicyclic cycloalkyl, dioxo-cyclohexyl, C₇₋₁₀ spirocyclic saturated carbocyclyl, 7-10 membered spirocyclic saturated heterocyclyl containing 1 heteroatom selected from nitrogen or a 5-6 membered saturated heterocyclyl containing 1 or 2 heteroatoms independently selected from nitrogen, oxygen, and sulfur, wherein the cycloalkyl, carbocyclyl, and heterocyclyl are substituted with (i) 1 occurrence of R⁷ and (ii) n occurrences of R⁸.

In certain embodiments, A² is a C₄₋₆ monocyclic cycloalkyl, C₅₋₇ bridged or fused bicyclic cycloalkyl, dioxo-cyclohexyl, C₇₋₁₀ spirocyclic saturated carbocyclyl, or a 5-6 membered saturated heterocyclyl containing 1 or 2 heteroatoms independently selected from nitrogen, oxygen, and sulfur, wherein the cycloalkyl, carbocyclyl, and heterocyclyl are substituted with (i) 1 occurrence of R⁷ and (ii) n occurrences of R⁸.

In certain embodiments, A² is a C₅₋₇ bridged or fused bicyclic cycloalkyl, wherein the cycloalkyl is substituted with (i) 1 occurrence of R⁷ and (ii) n occurrences of Ra. In certain embodiments, A² is a C₄₋₆ monocyclic cycloalkyl substituted with (i) 1 occurrence of R⁷ and (ii) n occurrences of R⁸. In certain embodiments, A² is dioxo-cyclohexyl substituted with (i) 1 occurrence of R⁷ and (ii) n occurrences of R⁸. In certain embodiments, A² is a C₅₋₆ monocyclic cycloalkyl, wherein the cycloalkyl is substituted with (i) 1 occurrence of R⁷ and (ii) n occurrences of R⁸. In certain embodiments, A² is cyclohexyl, cyclopentyl, or cyclobutyl, each of which is substituted with (i) 1 occurrence of R⁷ and (ii) n occurrences of R⁸. In certain embodiments, A² is cyclohexyl substituted with (i) 1 occurrence of R⁷ and (ii) n occurrences of R⁵. In certain embodiments, A² is cyclopentyl substituted with (i) 1 occurrence of R⁷ and (ii) n occurrences of R⁸.

In certain embodiments, A² is C₅₋₈ bridged bicyclic cycloalkyl substituted with (i) 1 occurrence of R⁷ and (ii) n occurrences of R⁵. In certain embodiments, A² is C₅₋₈ fused bicyclic cycloalkyl substituted with (i) 1 occurrence of R⁷ and (ii) n occurrences of R⁸. In certain embodiments, A² is C₅₋₇ bridged bicyclic cycloalkyl substituted with (i) 1 occurrence of R⁷ and (ii) n occurrences of R⁸. In certain embodiments, A² is C₅₋₇ fused bicyclic cycloalkyl substituted with (i) 1 occurrence of R⁷ and (ii) n occurrences of R⁸. In certain embodiments, A² is a 5-6 membered saturated heterocyclyl containing 1 or 2 heteroatoms independently selected from nitrogen, oxygen, and sulfur, wherein the heterocyclyl is substituted with (i) 1 occurrence of R⁷ and (ii) n occurrences of R⁸. In certain embodiments, A² is piperidinyl or pyrrolidinyl, each of which is substituted with (i) 1 occurrence of R⁷ and (ii) n occurrences of R⁸.

In certain embodiments, A² is a C₇₋₁₀ spirocyclic saturated carbocyclyl, wherein the carbocyclyl is substituted with (i) 1 occurrence of R⁷ and (ii) n occurrences of R⁸. In certain embodiments, A² is a C₇ spirocyclic saturated carbocyclyl, wherein the carbocyclyl is substituted with (i) 1 occurrence of R⁷ and (ii) n occurrences of R⁸.

In certain embodiments, A² is a 7-10 membered spirocyclic saturated heterocyclyl containing 1 heteroatom selected from nitrogen, wherein the heterocyclyl is substituted with (i) 1 occurrence of R⁷ and (ii) n occurrences of R³. In certain embodiments, A² is a 7-membered spirocyclic saturated heterocyclyl containing 1 heteroatom selected from nitrogen, wherein the heterocyclyl is substituted with (i) 1 occurrence of R⁷ and (ii) n occurrences of R⁸.

In certain embodiments, A² is dioxo-cyclohexyl. In certain embodiments, A² is

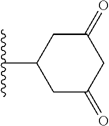 [SMILES: *C1CC(=O)CC(=O)C1]

In certain embodiments, A² is one of the following:

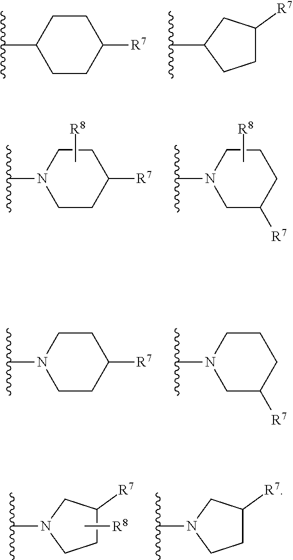

As generally defined above, X¹ is —N(R⁹)C(O)-Ψ, —C(O)N(R⁹)-Ψ, -(3-6 membered saturated heterocyclylene containing 1 or 2 heteroatoms selected from nitrogen)-C(O)-Ψ, —N(R⁹)C(R¹⁰)(R¹¹)-Ψ, —N(R⁹)-Ψ, —C(O)-Ψ, —(C(R¹⁰)(R¹¹))_{q}-Ψ, -(3-6 membered saturated heterocyclylene containing 1 or 2 heteroatoms selected from nitrogen)-C(R¹⁰)(R¹¹)-Ψ, —(C(R¹⁰)(R¹¹))ₚ—N(R⁹)-Ψ, or a covalent bond; wherein Ψ is a bond to A², and wherein the 3-6 membered saturated heterocyclylene is substituted with 0, 1, or 2 occurrences of C₁₋₄ alkyl. In certain embodiments, X¹ is —N(R⁹)C(O)-Ψ, —C(O)N(R⁹)-Ψ, —(C₁₋₃ alkylene)-N(R⁹)C(O)-Ψ, -(3-5 membered saturated heterocyclylene containing 1 or 2 heteroatoms selected from nitrogen)-C(O)-Ψ, —N(R⁹)C(R¹⁰)(R¹¹)-Ψ, —N(R⁹)-Ψ, —C(O)-Ψ, —C(R¹⁰)(R¹¹)-Ψ, -(3-6 membered saturated heterocyclylene containing 1 or 2 heteroatoms selected from nitrogen)-C(R¹⁰)(R¹¹)-Ψ, —C(R¹⁰)(R¹¹)—N(R⁹)-Ψ, or a covalent bond; wherein Ψ is a bond to A².

In certain embodiments, X¹ is —N(R⁹)C(O)-Ψ, —C(O)N(R⁹)-Ψ, —C₁₋₃ alkylene)-N(R⁹)C(O)-Ψ, -(3-5 membered saturated heterocyclylene containing 1 or 2 heteroatoms selected from nitrogen)-C(O)-Ψ, —N(R⁹)C(R¹⁰)(R¹¹)-Ψ, —N(R⁹)-Ψ, —C(O)-Ψ, —C(R¹⁰)(R¹¹)-Ψ, -(3-5 membered saturated heterocyclylene containing 1 or 2 heteroatoms selected from nitrogen)-C(R¹⁰)(R¹¹)-Ψ, —C(R¹⁰)(R¹¹)—N(R⁹)-Ψ, or a covalent bond; wherein w is a bond to A².

In certain embodiments, A² is selected from the groups depicted in the compounds in Table 1, below.

In certain embodiments, X¹ is —N(R⁹)C(O)-Ψ, wherein Ψ is a bond to A². In certain embodiments, X¹ is —(C₁₋₃ alkylene)-N(R⁹)C(O)-Ψ, wherein Ψ is a bond to A². In certain embodiments, X¹ is -(3-5 membered saturated heterocyclylene containing 1 or 2 heteroatoms selected from nitrogen)-C(O)-Ψ, wherein Ψ is a bond to A². In certain embodiments, X¹ is -(3-6 membered saturated heterocyclylene containing 1 or 2 heteroatoms selected from nitrogen)-C(O)-Ψ, wherein Ψ is a bond to A². In certain embodiments, X¹ is —N(R⁹)C(R¹⁰)(R¹¹)-Ψ, wherein Ψ is a bond to A². In certain embodiments, X¹ is —N(R⁹)-Ψ, wherein Ψ is a bond to A². In certain embodiments, X¹ is —C(O)-Ψ, wherein Ψ is a bond to A¹. In certain embodiments, X¹ is —C(R¹⁰)(R¹¹)-Ψ, wherein Ψ is a bond to A². In certain embodiments, X¹ is —(C(R¹⁰)(R¹¹))ₚ-Ψ, wherein Ψ is a bond to A². In certain embodiments, X¹ is —C(R¹⁰)(R¹¹)—N(R⁹)-Ψ, wherein Ψ is a bond to A². In certain embodiments, X¹ is —(C(R¹⁰)(R¹¹))ₚ—N(R⁹)-Ψ, wherein Ψ is a bond to A². In certain embodiments, X¹ is a -(3-6 membered saturated heterocyclylene containing 1 or 2 heteroatoms selected from nitrogen)-C(R¹⁰)(R¹¹)-Ψ, wherein Ψ is a bond to A². In certain embodiments, the 3-5 membered saturated heterocyclylene is substituted with 0, 1, or 2 occurrences of C₁₋₄ alkyl.

In certain embodiments, X¹ is —N(R⁹)C(O)-Ψ. In certain embodiments, X¹ is —(C₁₋₃ alkylene)-N(R⁹)C(O)-Ψ. In certain embodiments, X¹ is —(CH₂)—N(R⁹)C(O)-Ψ. In certain embodiments, X¹ is a -(3-5 membered saturated heterocyclylene containing 1 or 2 heteroatoms selected from nitrogen)-C(O)-Ψ. In certain embodiments, X¹ is -(3-6 membered saturated heterocyclylene containing 1 or 2 heteroatoms selected from nitrogen)-C(O)-Ψ. In certain embodiments, X¹ is a -(4-membered saturated heterocyclylene containing 1 heteroatom selected from nitrogen)-C(O)-Ψ. In certain embodiments, X¹ is

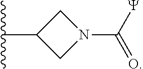

In certain embodiments, X¹ is selected from the groups depicted in the compounds in Table 1, below.

As generally defined above, Y¹ is N or —C(H)—. In certain embodiments, Y¹ is N. In certain embodiments, Y¹ is —C(H)—. In certain embodiments, Y¹ is selected from the groups depicted in the compounds in Table 1, below.

As generally defined above, m and n are independently 0, 1, 2, 3, or 4. In certain embodiments, m and n are independently 0, 1, 2, or 3. In certain embodiments, m and n are independently 0, 1, or 2. In certain embodiments, n is 1. In certain embodiments, n is 0. In certain embodiments, n is 2 In certain embodiments, n is 3. In certain embodiments, n is 4. In certain embodiments, n is a value according to that depicted in the compounds in Table 1, below.

In certain embodiments, m is 0. In certain embodiments, m is 1. In certain embodiments, m is 2. In certain embodiments, m is 3. In certain embodiments, m is 4. In certain embodiments, m is a value according to that depicted in the compounds in Table 1, below.

As generally defined above, p is 1, 2, 3, 4, 5, or 6. In certain embodiments, p is 1 or 2. In certain embodiments, p is 1 In certain embodiments, p is 2. In certain embodiments, p is 3 In certain embodiments, p is 4. In certain embodiments, p is 5. In certain embodiments, p is 6. In certain embodiments, p is a value according to that depicted in the compounds in Table 1, below.

In certain embodiments, the compound is represented by Formula Ia or a stereoisomer or pharmaceutically acceptable salt thereof:

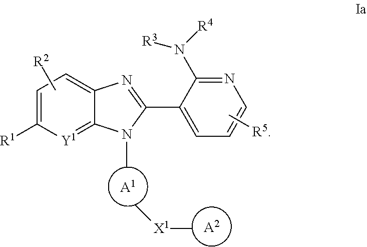

In certain embodiments, the compound is a compound of Formula Ib, Ic, Id, Ie, If, or Ig, or a stereoisomer or pharmaceutically acceptable salt thereof:

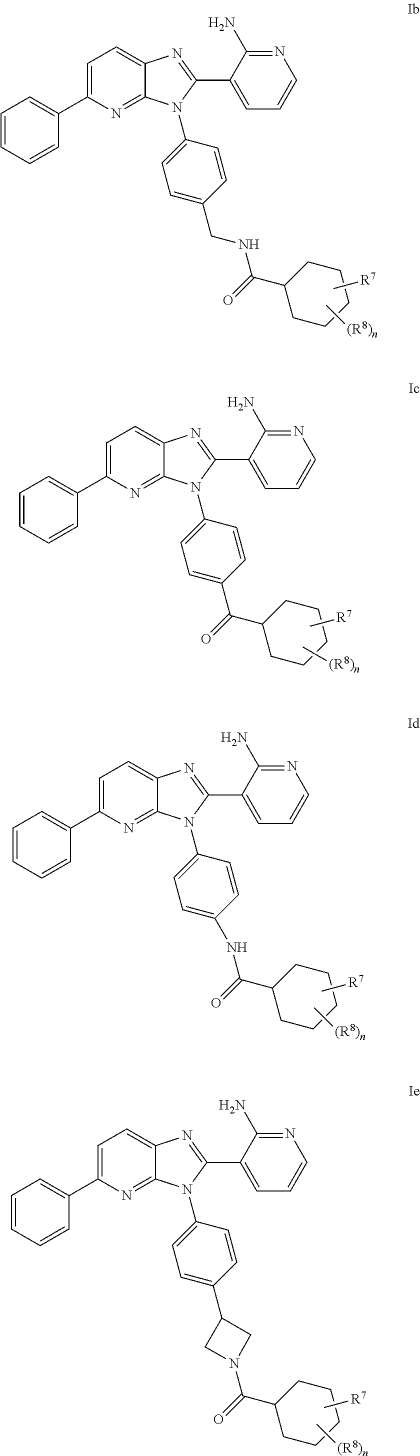

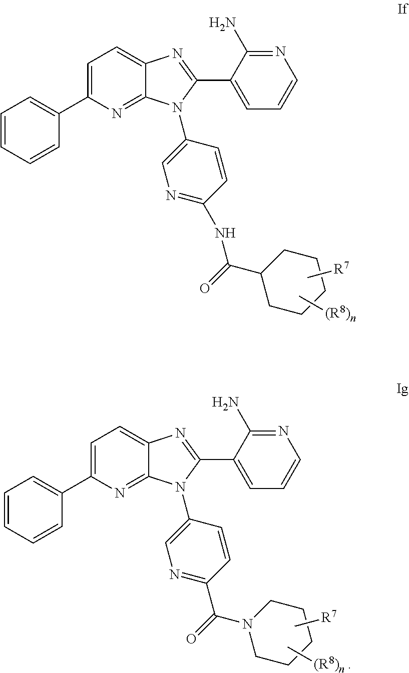

In certain embodiments, the compound is a compound of Formula Ih, Ii, Ij, Ik, Il, or Im, or a stereoisomer or pharmaceutically acceptable salt thereof:

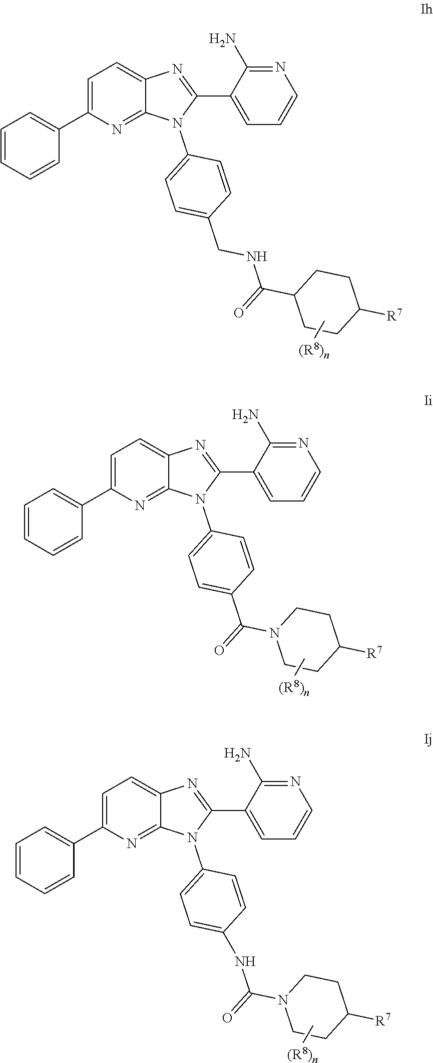

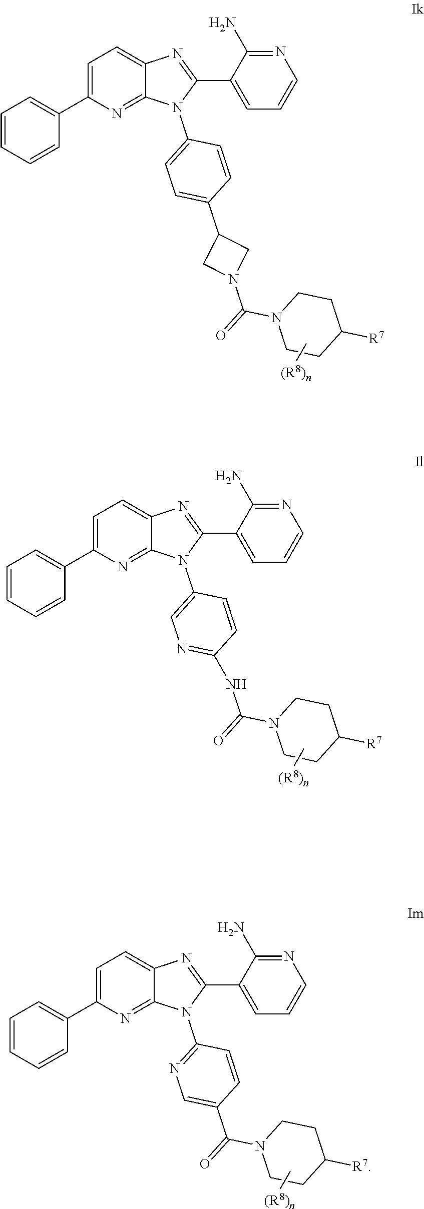

Another aspect of the invention provides a compound represented by Formula Il:

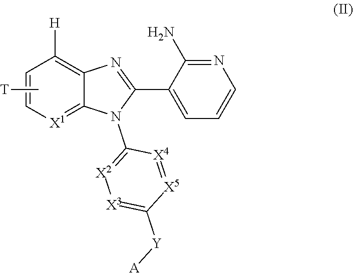

or a stereoisomer or pharmaceutically acceptable salt thereof, wherein:
T is hydrogen, halo, cyano, C(═O)NH₂, C₁₋₆ alkyl, C₁₋₆ alkoxyl, C₃₋₁₂ cycloalkyl, 3-12 membered heterocyclyl, C₆₋₁₀ aryl, or 5-9 membered heteroaryl, wherein the C₁₋₆ alkyl, C₁₋₆ alkoxyl, C₆₋₁₀ aryl, or 5-9 membered heteroaryl are optionally substituted with one or more occurrences of R^{T};
R^{T} is independently for each occurrence halo, C₁₋₆ alkyl, cyano, or NHC(═O)R^{T1};
R^{T1} is C₆₋₁₀ aryl or C₁₋₆ alkyl;
X¹ is N or CH;
X² and X³ are independently for each occurrence N or CR^{X};
X⁴ and X⁵ are CR^{X};
R^{X} is independently for each occurrence hydrogen, C₁₋₆ alkyl, or halo;
Y is absent, or Y is selected from the group consisting of —C(R^{Y1})₂—, —R^{Y2}C(R^{Y1})₂—*, —C(R^{Y1})₂R^{Y2}—*, —NR^{Y1}—, —C(R^{Y1})₂NR^{Y1}—*, —NR^{Y1}C(R^{Y1})₂—*, —CH₂NR^{Y1}(C═O)—*, —(C═O)NR^{Y1}CH₂-*, —NR^{Y1}C(═O)—*, —C(═O)NR^{Y1}—*, —C(═O)—, —R^{Y2}C(═O)—*, and —C(═O)R^{Y2}—*, wherein * denotes an attachment to A:
R^{Y1} is independently for each occurrence hydrogen or C₁₋₆ alkyl;
R^{Y2} is 3-12 membered heterocyclyl;
A is C₃₋₁₂ cycloalkyl or 3-12 membered heterocyclyl, wherein the C₃₋₁₂ cycloalkyl or 3-12 membered heterocyclyl are substituted with one or more occurrences of R^{A};
R^{A} is independently for each occurrence oxo, halo, C₁₋₆ alkyl, —(C₀₋₆ alkylene)-C(═O)OR^{A1}, C(═O)N(R^{A1})₂, NHC(═O)R^{A1}, NHC(═O)OR^{A1}, OC(═O)N(R^{A1})₂, S(═O)₂OR^{A1}, or S(═O)₂N(R^{A1})₂; and
R^{A1} is independently for each occurrence hydrogen or C₁₋₆ alkyl,
wherein when Y is absent, A is not cyclobutyl, azetidinyl, or oxetanyl.

Another aspect of the invention provides a compound represented by Formula II:

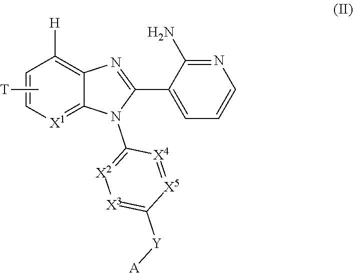

or a stereoisomer or pharmaceutically acceptable salt thereof, wherein:
T is hydrogen, halo, cyano, C(═O)NH₂, C₁₋₆ alkyl, C₁₋₆ alkoxyl, C₃₋₁₂ cycloalkyl, 3-12 membered heterocyclyl, C₆₋₁₀ aryl, or 5-9 membered heteroaryl, wherein the C₁₋₆ alkyl, C₁₋₆ alkoxyl, C₆₋₁₀ aryl, or 5-9 membered heteroaryl are optionally substituted with one or more occurrences of R^{T};
R^{T} is independently for each occurrence halo, C₁₋₆ alkoxyl, C₁₋₆ alkyl, cyano, or NHC(═O)R^{T1};
R^{T1} is C₆₋₁₀ aryl or C₁₋₆ alkyl;
X¹ is N or CH;
X² and X³ are independently for each occurrence N or CR^{X};
X⁴ and X⁵ are CR^{X};
R^{N} is independently for each occurrence hydrogen, C₁₋₆ alkyl, or halo;
Y is absent, or Y is selected from the group consisting of —(C(R^{Y1})₂)_{q}-, —R^{Y2}C(R^{Y1})₂—*, —C(R^{Y1})₂R^{Y2}—*, —NR^{Y1}—, —(C(R^{Y1})₂)_{q}NR^{Y1}—*, —NR^{Y1}C(R^{Y1})₂—*, —CH₂NR^{Y1}(C═O)—*, —(C═O)NR^{Y1}CH₂—*, —NR^{Y1}C(═O)—*, —C(═O)NR^{Y1}—*, —C(═O)—, —R^{Y2}C(═O)—*, and —C(═O)R^{Y2}—*, wherein * denotes an attachment to A;
R^{Y1} is independently for each occurrence hydrogen, C₁₋₆ alkyl, or C₁₋₆ haloalkyl;
R^{Y2} is 3-12 membered heterocyclyl, wherein the 3-12 membered heterocyclyl is optionally substituted with one or more occurrences of C₁₋₆ alkyl;
q is 1, 2, 3, 4,5 or 6;
A is C₃₋₁₂ cycloalkyl or 3-12 membered heterocyclyl, wherein the C₃₋₁₂ cycloalkyl or 3-12 membered heterocyclyl are substituted with one or more occurrences of R^{A};
R^{A} is independently for each occurrence oxo, halo, C₁₋₆ alkyl, —(C₀₋₆ alkylene)-C(═O)OR^{A1}, C(═O)N(R^{A1})₂, NHC(═O)R^{A1}, NHC(═O)OR^{A1}, OC(═O)N(R^{A1})₂, S(═O)₂OR^{A1}, or S(═O)₂N(R^{A1})₂; and
R^{A1} is independently for each occurrence hydrogen or C₁₋₆ alkyl,
   wherein when Y is absent, A is not cyclobutyl, azetidinyl, or oxetanyl.

In certain embodiments, the compound is represented by Formula II:

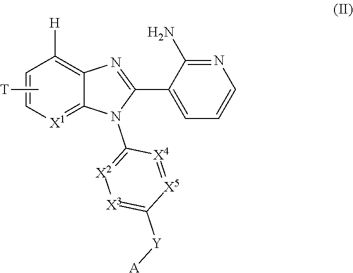

or a stereoisomer or pharmaceutically acceptable salt thereof, wherein:
T is hydrogen, halo, cyano, C(═O)NH₂, C₁₋₆ alkyl, C₁₋₆ alkoxyl, C₃₋₁₂ cycloalkyl, 3-12 membered heterocyclyl, C₆₋₁₀ aryl, or 5-9 membered heteroaryl, wherein the C₁₋₆ alkyl, C₁₋₆ alkoxyl, C₆₋₁₀ aryl, or 5-9 membered heteroaryl are optionally substituted with one or more occurrences of R^{T};
R^{T} is independently for each occurrence halo, C₁₋₆ alkyl, cyano, or NHC(═O)R^{T1};
R^{T1} is C₆₋₁₀ aryl or C₁₋₆ alkyl;
X¹ is N or CH;
X² and X³ are independently for each occurrence N or CR^{X};
X⁴ and X⁵ are CR^{X};
R^{X} is independently for each occurrence hydrogen, C₁₋₆ alkyl, or halo; Y is absent, or Y is selected from the group consisting of —C(R^{Y1})₂—, —R^{Y2}C(R^{Y1})₂—*, —C(R^{Y1})₂R^{Y2}—*, —NR^{Y1}—, —C(R^{Y1})₂NR^{Y1}—*, —NR^{Y1}C(R^{Y1})₂—*, —CH₂NR^{Y1}(C═O)—*, —(C═O)NR^{Y1}CH₂—*, —NR^{Y1}C(═O)—*, —C(═O)NR^{Y1}—*, —C(═O)—, —R^{Y2}C(═O)—*, and —C(═O)R^{Y2}—*, wherein * denotes an attachment to A;
R^{Y1} is independently for each occurrence hydrogen or C₁₋₆ alkyl;
R^{Y2} is 3-12 membered heterocyclyl;
A is C₅₋₁₂ cycloalkyl or 5-12 membered heterocyclyl, wherein the C₃₋₁₂ cycloalkyl or 3-12 membered heterocyclyl are substituted with one or more occurrences of R^{A};
R^{A} is independently for each occurrence oxo, halo, C₁₋₆ alkyl, —(C₀₋₆ alkylene)-C(═O)OR^{A1}, C(═O)N(R^{A1})₂, NHC(═O)R^{A1}, NHC(═O)OR^{A1}, OC(═O)N(R^{A1})₂, S(═O)₂OR^{A1}, or S(═O)₂N(R^{A1})₂; and
R^{A1} is independently for each occurrence hydrogen or C₁₋₆ alkyl.

As generally defined above, T is hydrogen, halo, cyano, C(═O)NH₂, C₁₋₆ alkyl, C₁₋₆ alkoxyl, C₃₋₁₂ cycloalkyl, 3-12 membered heterocyclyl, C₆₋₁₀ aryl, or 5-9 membered heteroaryl, wherein the C₁₋₆ alkyl, C₁₋₆ alkoxyl, C₆₋₁₀ aryl, or 5-9 membered heteroaryl are optionally substituted with one or more occurrences of R^{T}. In certain embodiments, T is phenyl, C₁₋₄ alkyl, C₅₋₆cycloalkyl, C₁₋₃ alkoxy, 5-6 membered heteroaryl, or 6-membered heterocyclyl, wherein the phenyl. C₁₋₃ alkoxy, or 5-6 membered heteroaryl are optionally substituted with one or more occurrences of R^{T}. In certain embodiments, T is phenyl, methyl, tert-butyl, cyclopentyl, cyclohexyl, methoxy, propoxy, morpholinyl, tetrahydropyranyl, pyrazolyl, or pyridinyl, wherein the phenyl, methoxy, or pyrazolyl are optionally substituted with one or more occurrences of Rr.

In certain embodiments, T is hydrogen. In certain embodiments, T is halo. In certain embodiments, T is cyano. In certain embodiments, T is C(═O)NH₂. In certain embodiments, T is C₁₋₆alkyl. In certain embodiments, T is C₁₋₆alkoxyl. In certain embodiments, T is C₃₋₁₂ cycloalkyl. In certain embodiments, T is 3-12 membered heterocyclyl. In certain embodiments, T is C₆₋₁₀ aryl. In certain embodiments, T is 5-9 membered heteroaryl.

In certain embodiments, T is phenyl optionally substituted with one or more occurrences of R^{T}. In certain embodiments, T is phenyl substituted with one or more occurrences of R^{T}. In certain embodiments, T is phenyl. In certain embodiments, T is tert-butyl. In certain embodiments, T is cyclohexyl. In certain embodiments, T is cyclopentyl. In certain embodiments, T is C, alkoxyl. In certain embodiments, T is C₄ alkoxyl. In certain embodiments, T is 5-membered heteroaryl. In certain embodiments, T is pyridinyl. In certain embodiments, T is 6-membered heterocyclyl. In certain embodiments, T is methoxy optionally substituted with one or more occurrences of R^{T}. In certain embodiments, T is methoxy substituted with one or more occurrences of R^{T}. In certain embodiments. T is methoxy. In certain embodiments, T is propoxy. In certain embodiments, T is morpholinyl. In certain embodiments, T is tetrahydropyranyl. In certain embodiments, T is pyrazolyl optionally substituted with one or more occurrences of R^{T}. In certain embodiments, T is pyrazolyl substituted with one or more occurrences of Rr. In certain embodiments, T is pyrazolyl. In certain embodiments, T is pyridinyl.

As generally defined above, R^{T} is independently for each occurrence halo, C₁₋₆ alkoxyl, C₁₋₆ alkyl, cyano, or NHC(═O)R^{T1}. In certain embodiments, R_{T} is independently for each occurrence halo, C₁₋₆ alkyl, cyano, or NHC(═O)R^{T1}. In certain embodiments, R^{T} is halo, C₁₋₆ alkoxyl, C₁₋₆ alkyl, or cyano. In certain embodiments, R^{T} is halo, C₁₋₆ alkyl, or cyano. In certain embodiments, R_{T} is fluoro, methoxy, methyl, or cyano. In certain embodiments, R^{T} is fluoro, methyl, or cyano. In certain embodiments, R^{T} is halo. In certain embodiments, R^{T} is fluoro. In certain embodiments, R^{T} is chloro. In certain embodiments, R^{T} is bromo. In certain embodiments, R^{T} is iodo. In certain embodiments, R^{T} is C₁₋₆ alkyl. In certain embodiments, R^{T} is methyl. In certain embodiments, R_{T} is cyano. In certain embodiments, R^{T} is C₁₋₆ alkoxyl. In certain embodiments, R^{T} is methoxy. In certain embodiments, R^{T} is NHC(═O)R^{T1}.

As generally defined above, R^{T1} is C₆₋₁₀ aryl or C₁₋₆ alkyl. In certain embodiments, R^{T1} is C₆₋₁₀ aryl. In certain embodiments, R^{T1} is C₁₋₆ alkyl.

As generally defined above, X¹ is N or CH. In certain embodiments, X¹ is N. In certain embodiments, X¹ is CH.

As generally defined above, X² and X³ are independently for each occurrence N or CR^{X}. In certain embodiments, X² is N. In certain embodiments, X³ is N. In certain embodiments, X² is CR^{X}. In certain embodiments, X³ is CR^{X}. In certain embodiments, X² is N and X³ is CR^{X}. In certain embodiments, X² is CR^{X} and X³ is N. In certain embodiments, X² is CR^{X} and X³ is CR^{X}.

As generally defined above, X⁴ and X⁵ are CR^{X}. In certain embodiments, X⁴ is CR^{X}. In certain embodiments, X⁵ is CR^{X}.

In certain embodiments, X² is CH, X³ is CH, X⁴ is CH, and X⁵ is CH. In certain embodiments, X² is C(F), X³ is CH, X⁴ is CH, and X⁵ is CH. In certain embodiments, X² is CH, X³ is CH, X⁴ is CH, and X⁵ is C(F). In certain embodiments, X² is CH, X³ is N, X⁴ is CH, and X⁵ is CH. In certain embodiments, X² is CH, X³ is C(F), X⁴ is CH, and X⁵ is CH. In certain embodiments, X² is C(CH₃), X³ is CH, X⁴ is CH, and X⁵ is CH. In certain embodiments, X² is C(CH₃), X³ is C(F), X⁴ is CH, and X⁵ is CH. In certain embodiments, X² is C(CH₃), X³ is N, X⁴ is CH, and X⁵ is CH. In certain embodiments, X² is N, X³ is C(CH₃), X⁴ is CH, and X⁵ is CH.

As generally defined above, R^{X} is independently for each occurrence hydrogen, C₁₋₆ alkyl, or halo. In certain embodiments, R^{X} is hydrogen, methyl, or fluoro. In certain embodiments, R^{X} is hydrogen. In certain embodiments. R^{X} is C₁₋₆ alkyl. In certain embodiments, R^{X} is methyl. In certain embodiments, R^{X} is halo. In certain embodiments, R^{X} is fluoro. In certain embodiments, R^{X} is chloro. In certain embodiments, R^{X} is bromo. In certain embodiments, R^{X} is iodo.

As generally defined above, Y is absent, or Y is selected from the group consisting of —(C(R^{Y1})₂)—, —R^{Y2}C(R^{Y1})₂—*, —C(R^{Y1})₂R^{Y2}—*, —NR^{Y1}—, —(C(R^{Y1})₂)_{q}NR^{Y1}—*, —NR^{Y1}C(R^{Y1})₂—*, —CH₂NR^{Y1}(C═O)—*, —(C═O)NR^{Y1}CH₂—*, —NR^{Y1}C(═O)—*, —C(═O)NR^{Y1}—*, —C(═O)—, —R^{Y2}C(═O)—*, and —C(═O)R^{YZ}—*, wherein * denotes an attachment to A. In certain embodiments, Y is absent, or Y is selected from the group consisting of —C(R^{Y1})₂—, —R^{Y2}C(R^{Y1})₂—*, —C(R^{Y1})₂R^{Y2}—*, —NR^{Y1}—, —C(R^{Y1})₂NR^{Y1}—*, —NR^{Y1}C(R^{Y1})₂—*, —CH₂NR^{Y1}(C═O)—*, —(C═O)NR^{Y1}CH₂—*, —NR^{Y1}C(═O)—*, —C(═O)NR^{Y1}—*, —C(═O)—, —R^{Y2}C(═O)—*, and —C(═O)R^{Y2}—*, wherein * denotes an attachment to A. In certain embodiments. Y is selected from the group consisting of —C(R^{Y1})₂—, —R^{Y2}C(R^{Y1})₂—*, —C(R^{Y1})₂R^{Y2}—*, —NR^{Y1}—, —C(R^{Y1})₂NR′—*, —NR^{Y1}C(R^{Y1})₂—*, —CH₂NR^{Y1}C(═O)—*, —(C═O)NR^{Y1}CH₂—*, —NR^{Y1}C(═O)—*, —C(═O)NR^{Y1}—*, —C(═O)—, —R^{Y2}C(═O)—*, and —C(═O)R^{Y1}═-*, wherein * denotes an attachment to A. In certain embodiments, Y is —NHC(═O)—*, —NHCH₂—*, —NH—, —C(═O)NH—*, —CH₂—, —CH₂CH₂—, —CH₂NH—*, —CH₂CH₂N(CH₃)—*, azetidinyl-CH₂—*, azetidinyl-(C═O)—*, —CH₂NH(C═O)—*, azetidinyl-CH(CH₃)*, azetidinyl-CH(CH(CH₃)₂)—*, piperidinyl-CH₂—*, pyrrolidinyl-CH₂—*, —CH(CH₃)—*, —CH((CH₃)NH—*, —C(CH₃)NH—*, —CH₂N(CH)—, or —C(═O)—. In certain embodiments, Y is —NHC(═O)—*, —NHCH₂—*, —NH—, —C(═O)NH—*, —CH₂—, —CH₂NH—*, azetidinyl-CH₂—*, azetidinyl-(C═O)—*, —CH₂NH(C═O)—*, azetidinyl-CH(CHI)—*, azetidinyl-CH(CH(CH₃)₂)—*, piperidinyl-CH₂—*, pyrrolidinyl-CH₂—*, —CH(CH₄)—*, —CH(CH₃)NH—*, —C(CH₃)₂NH—*, —CH₂N(CH₃)—, or —C(═O)—.

In certain embodiments, Y is —(C(R^{Y1})₂)_{q}—. In certain embodiments, Y is —C(R^{Y1})₂—. In certain embodiments, Y is —R^{Y2}C(R^{Y1})₂—*. In certain embodiments. Y is —C(R^{Y1})₂R^{Y2}—*. In certain embodiments, Y is —NR^{Y1}—. In certain embodiments, Y is —(C(R^{Y1})₂)_{q}NR^{Y1}—*. In certain embodiments, Y is —C(R^{Y1})₂NR^{Y1}—*. In certain embodiments, Y is —NR^{Y1}C(R^{Y1})₂—*. In certain embodiments, Y is —CH₂NR^{Y1}(C═O)—*. In certain embodiments, Y is —(C═O)NR^{Y1}CH₂—*. In certain embodiments, Y is —NR^{Y1}C(═O)—*. In certain embodiments, Y is —C(═O)NR^{Y1}—*. In certain embodiments, Y is —C(═O)—. In certain embodiments, Y is —R^{Y2}C(═O)—*. In certain embodiments, Y is —C(═O)R^{Y2}—*.

In certain embodiments, Y is —NHC(═O)—*. In certain embodiments, Y is —NHCH₂—*. In certain embodiments, Y is —NH—. In certain embodiments, Y is —C(═O)NH—*. In certain embodiments, Y is —CH₂—. In certain embodiments, Y is —CH₂CH₂—. In certain embodiments, Y is —CH₂NH—*. In certain embodiments, Y is —CH₂CH₂N(CH₃)—*. In certain embodiments, Y is azetidinyl-CH₂—*. In certain embodiments, Y is azetidinyl-(C═O)—*. In certain embodiments, Y is —CH₂NH(C═O)—*. In certain embodiments, Y is azetidinyl-CH(CH₃)—*. In certain embodiments, Y is azetidinyl-CH(CH(CH₃)₂)—*. In certain embodiments, Y is piperidinyl-CH₂—*. In certain embodiments, Y is pyrrolidinyl-CH₂—*. In certain embodiments, Y is —CH(CH₃)—*. In certain embodiments, Y is —CH(CH₃)NH—*. In certain embodiments, Y is —C(CH₃)₂NH—*. In certain embodiments, Y is —CH₂N(CH₃)—*.

As generally defined above, R¹² is 3-12 membered heterocyclyl, wherein the 3-12 membered heterocyclyl is optionally substituted with one or more occurrences of C₁₋₆ alkyl. In certain embodiments, R^{Y2} is 3-12 membered heterocyclyl. In certain embodiments, R¹² is 3-12 membered heterocyclyl substituted with one or more occurrences of C₁₋₆ alkyl. In certain embodiments, R^{Y2} is 4-membered heterocyclyl. In certain embodiments, R^{Y2} is 5-membered heterocyclyl. In certain embodiments, R^{Y2} is 6-membered heterocyclyl. In certain embodiments, R^{Y2} is azetidinyl, piperidinyl, or pyrrolidinyl. In certain embodiments, R^{Y2} is azetidinyl. In certain embodiments, R^{Y2} is piperidinyl. In certain embodiments, R^{Y2} is pyrrolidinyl.

As generally defined above, R^{Y1} is independently for each occurrence hydrogen, C₁₋₆ alkyl, or C₁₋₆ haloalkyl. In certain embodiments, R^{Y1} is independently for each occurrence hydrogen or C₁₋₈ alkyl. In certain embodiments, R^{Y1} is hydrogen, methyl, or iso-butyl. In certain embodiments, R^{Y1} is hydrogen. In certain embodiments, R^{Y1} is C₁₋₆ alkyl. In certain embodiments, R^{Y1} is methyl. In certain embodiments, R^{Y1} is iso-butyl. In certain embodiments, R^{Y1} is C₁₋₆ haloalkyl.

As generally defined above, q is 1, 2, 3, 4, 5, or 6. In certain embodiments, q is 1 or 2. In certain embodiments, q is 1. In certain embodiments, q is 2. In certain embodiments, q is 3. In certain embodiments, q is 4 In certain embodiments, q is 5. In certain embodiments, q is 6.

As generally defined above, A is C₃₋₁₂ cycloalkyl or 3-12 membered heterocyclyl, wherein the C₃₋₁₂ cycloalkyl or 3-12 membered heterocyclyl are substituted with one or more occurrences of R^{A}; or when Y is absent, A is C₅₋₁₂ cycloalkyl or 5-12 membered heterocyclyl, wherein the C₅₋₁₂ cycloalkyl or 5-12 membered heterocyclyl are substituted with one or more occurrences of R^{A}. In certain embodiments, A is C3.12 cycloalkyl or 3-12 membered heterocyclyl, wherein the C₃₋₁₂ cycloalkyl or 3-12 membered heterocyclyl are substituted with one or more occurrences of R^{A}. In certain embodiments, A is C₃₋₁₂ cycloalkyl substituted with one or more occurrences of R^{A}. In certain embodiments, A is 3-12 membered heterocyclyl substituted with one or more occurrences of R^{A} In certain embodiments, A is C₃₋₁₂ cycloalkyl. In certain embodiments, A is 3-12 membered heterocyclyl. In certain embodiments, A is C₅₋₁₂cycloalkyl or 5-12 membered heterocyclyl, wherein the C₃₋₁₂cycloalkyl or 3-12 membered heterocyclyl are substituted with one or more occurrences of R^{A}.

In certain embodiments, A is C₅₋₇ cycloalkyl or 5-7 membered heterocyclyl, wherein the C₅₋₇ cycloalkyl or 5-7 membered heterocyclyl are substituted with one or more occurrences of R⁴. In certain embodiments, A is cyclopentyl, bicyclo[1.1.1]pentyl, cyclohexyl, bicyclo[2.1.1]hexyl, bicyclo[3.1.0]hexyl, spiro[3.3]heptyl, pyrrolidinyl, piperidinyl, or 2-azaspiro[3.3]heptyl, wherein the cyclopentyl, bicyclo[1.1.1]pentyl, bicyclo[2.1.1]hexyl, bicyclo[3.1.0]hexyl, spiro[3.3]heptyl, pyrrolidinyl, piperidinyl, or 2-azaspiro[3.3]heptyl are substituted with one or more occurrences of R^{A}.

In certain embodiments, A is cyclopentyl substituted with one or more occurrences of R^{A}. In certain embodiments, A is cyclopentyl substituted with one occurrence of R^{A}. In certain embodiments, A is cyclopentyl substituted with one occurrence of —(C₀₋₆ alkylene)-C(═O)OR^{A1}. In certain embodiments, A is cyclopentyl substituted with —(C₀₋₆ alkylene)-C(═O)OR^{A1}. In certain embodiments, A is cyclopentyl substituted with one occurrence of —C(═O)OH. In certain embodiments, A is cyclopentyl substituted with —C(═O)OH. In certain embodiments, A is cyclopentyl substituted with —CH(CH₃)C(═O)OH.

In certain embodiments, A is bicyclo[1.1.1]pentyl substituted with one or more occurrences of R^{A}. In certain embodiments, A is bicyclo[1.1.1]pentyl substituted with one occurrence of R^{A}. In certain embodiments, A is bicyclo[1.1.1]pentyl substituted with one occurrence of —(C₀₋₆ alkylene)-C(═O)OR^{A1}. In certain embodiments, A is bicyclo[1.1.1]pentyl substituted with —(C₀₋₆ alkylene)-C(═O)OR^{A1}. In certain embodiments, A is bicyclo[1.1.1]pentyl substituted with one occurrence of —C(═O)OH. In certain embodiments, A is bicyclo[1.1.1]pentyl substituted with —C(═O)OH.

In certain embodiments, A is cyclohexyl substituted with one or more occurrences of R^{A}. In certain embodiments, A is cyclohexyl substituted with one occurrence of R^{A}. In certain embodiments, A is cyclohexyl substituted with one occurrence of —(C₀₋₆ alkylene)-C(═O)OR^{A1}. In certain embodiments, A is cyclohexyl substituted with —(C₀₋₆ alkylene)-C(═O)OR^{A1}. In certain embodiments, A is cyclohexyl substituted with one occurrence of —C(═O)OH. In certain embodiments, A is cyclohexyl substituted with —C(—O)OH. In certain embodiments, A is cyclohexyl substituted with —C(═O)OCH₃. In certain embodiments, A is cyclohexyl substituted with —C(═O)NH₂. In certain embodiments, A is cyclohexyl substituted with two occurrence of R^{A}. In certain embodiments, A is cyclohexyl substituted with two oxo. In certain embodiments, A is cyclohexyl substituted with one occurrence of —C(═O)OH and one occurrence of halo. In certain embodiments, A is cyclohexyl substituted with one occurrence of —C(═O)OH and one occurrence of fluoro. In certain embodiments, A is cyclohexyl substituted with one occurrence of —C(═O)OH and one occurrence of C₁₋₆ alkyl. In certain embodiments, A is cyclohexyl substituted with one occurrence of —C(═O)OH and one occurrence of methyl. In certain embodiments, A is cyclohexyl substituted with three occurrences of R^{A}. In certain embodiments, A is cyclohexyl substituted with one occurrence of —C(═O)OH and two occurrences of C₁₋₆ alkyl. In certain embodiments, A is cyclohexyl substituted with one occurrence of —C(═O)OH and two occurrences of methyl.

In certain embodiments, A is bicyclo[2.1.1]hexyl substituted with one or more occurrences of R^{A}. In certain embodiments, A is bicyclo[2.1.1]hexyl substituted with one occurrence of R^{A}. In certain embodiments, A is bicyclo[2.1.1]hexyl substituted with one occurrence of —(C₀₋₆ alkylene)-C(═O)OR^{A1}. In certain embodiments, A is bicyclo[2.1.1]hexyl substituted with —(C₀₋₆ alkylene)-C(═O)OR^{A1}. In certain embodiments, A is bicyclo[2.1.1]hexyl substituted with one occurrence of —C(═O)OH. In certain embodiments, A is bicyclo[2.1.1]hexyl substituted with —C(═O)OH.

In certain embodiments, A is bicyclo[3.1.0]hexyl substituted with one or more occurrences of R^{A}. In certain embodiments, A is bicyclo[3.1.0]hexyl substituted with one occurrence of R^{A}. In certain embodiments, A is bicyclo[3.1.0]hexyl substituted with one occurrence of —(C₀₋₆ alkylene)-C(═O)OR^{A1}. In certain embodiments, A is bicyclo[3.1.0]hexyl substituted with —(C₀₋₆ alkylene)-C(═O)OR^{A1}. In certain embodiments, A is bicyclo[3.1.0]hexyl substituted with one occurrence of —C(═O)OH. In certain embodiments, A is bicyclo[3.1.0]hexyl substituted with —C(═O)OH.

In certain embodiments, A is cycloheptyl substituted with one or more occurrences of R^{A}. In certain embodiments, A is cycloheptyl substituted with one occurrence of R^{A}. In certain embodiments, A is cycloheptyl substituted with one occurrence of —(C₀₋₆ alkylene)-C(═O)OR^{A1}. In certain embodiments, A is cycloheptyl substituted with —(C₀₋₆ alkylene)-C(═O)OR^{A1}. In certain embodiments, A is cycloheptyl substituted with one occurrence of —C(═O)OH.

In certain embodiments, A is spiro[3.3]heptyl substituted with one or more occurrences of R^{A}. In certain embodiments, A is spiro[3.3]heptyl substituted with one occurrence of R^{A}. In certain embodiments, A is spiro[3.3]heptyl substituted with one occurrence of —(C₁₋₆ alkylene)-C(═O)OR^{A1}. In certain embodiments, A is spiro[3.3]heptyl substituted with —(C₀₋₆ alkylene)-C(═O)OR^{A1}. In certain embodiments, A is spiro[3.3]heptyl substituted with one occurrence of —C(═O)OH.

In certain embodiments, A is piperidinyl substituted with one or more occurrences of R^{A}. In certain embodiments, A is piperidinyl substituted with one occurrence of R^{A}. In certain embodiments, A is piperidinyl substituted with one occurrence of —(C₀₋₆ alkylene)-C(═O)OR^{A1}. In certain embodiments, A is piperidinyl substituted with —(C₀₋₆ alkylene)-C(═O)OR^{A1}. In certain embodiments, A is piperidinyl substituted with one occurrence of —C(═O)OH. In certain embodiments, A is piperidinyl substituted with —C(═O)OH. In certain embodiments, A is piperidinyl substituted with —C(CH₃)₂C(═O)OH. In certain embodiments, A is piperidinyl substituted with —CH₂C(═O)OH. In certain embodiments, A is piperidinyl substituted with —CH(CH₃)C(═O)OH. In certain embodiments, A is piperidinyl substituted with —C(═O)NH₂. In certain embodiments, A is piperidinyl substituted with two occurrences of R^{A}. In certain embodiments, A is piperidinyl substituted with one occurrence of —C(═O)OH and one occurrence of C₁₋₆ alkyl. In certain embodiments, A is piperidinyl substituted with one occurrence of —C(═O)OH and one occurrence of methyl. In certain embodiments, A is piperidinyl substituted with three occurrences of R^{A}. In certain embodiments, A is piperidinyl substituted with one occurrence of —C(═O)OH and two occurrences of methyl. In certain embodiments, A is piperidinyl substituted with one occurrence of —C(═O)OH and two occurrences of halo. In certain embodiments, A is piperidinyl substituted with one occurrence of —C(═O)OH and two occurrences of fluoro. In certain embodiments, A is piperidinyl substituted with one occurrence of —C(═O)OH and two occurrences of C₁₋₆ alkyl. In certain embodiments, A is piperidinyl substituted with one occurrence of —C(═O)OH and two occurrences of methyl.

In certain embodiments, A is pyrrolidinyl substituted with one or more occurrences of R^{A}. In certain embodiments, A is pyrrolidinyl substituted with one occurrence of R^{A}. In certain embodiments, A is pyrrolidinyl substituted with one occurrence of —(C₀₋₆ alkylene)-C(═O)OR^{A1}. In certain embodiments, A is pyrrolidinyl substituted with —(C₀₋₆ alkylene)-C(═O)OR^{A1}. In certain embodiments, A is pyrrolidinyl substituted with one occurrence of —C(═O)OH. In certain embodiments, A is pyrrolidinyl substituted with —C(═O)OH. In certain embodiments, A is pyrrolidinyl substituted with —CH(CH₃)C(═O)OH. In certain embodiments, A is pyrrolidinyl substituted with —C(CH₃)₂C(═O)OH. In certain embodiments, A is pyrrolidinyl substituted with —CH₂C(═O)OH.

In certain embodiments, A is 7-membered heterocyclyl substituted with one or more occurrences of R^{A}. In certain embodiments, A is 7-membered heterocyclyl substituted with one occurrence of R^{A}. In certain embodiments, A is 7-membered heterocyclyl substituted with one occurrence of —(C₀₋₆ alkylene)-C(═O)OR^{A1}. In certain embodiments, A is 7-membered heterocyclyl substituted with —(C₀₋₆ alkylene)-C(═O)OR^{A1}. In certain embodiments, A is 7-membered heterocyclyl substituted with one occurrence of —C(═O)OH.

In certain embodiments, A is 2-azaspiro[3.3]heptyl substituted with one or more occurrences of R^{A}. In certain embodiments, A is 2-azaspiro[3.3]heptyl substituted with one occurrence of R^{A}. In certain embodiments, A is 2-azaspiro[3.3]heptyl substituted with one occurrence of —(C₀₋₆ alkylene)-C(═O)OR^{A1}. In certain embodiments, A is 2-azaspiro[3.3]heptyl substituted with —(C₀₋₆ alkylene)-C(═O)OR^{A1}. In certain embodiments, A is 2-azaspiro[3.3]heptyl substituted with one occurrence of —C(═O)OH.

In certain embodiments, Y is absent. In certain embodiments, when Y is absent, A is not cyclobutyl, azetidinyl, or oxetanyl. In certain embodiments, when Y is absent, A is C₅₋₁₂ cycloalkyl or 5-12 membered heterocyclyl, wherein the C₅₋₁₂ cycloalkyl or 5-12 membered heterocyclyl are substituted with one or more occurrences of R^{A}. In certain embodiments, when Y is absent, A is C₅₋₁₂ cycloalkyl substituted with one or more occurrences of R^{A}. In certain embodiments, when Y is absent, A is 5-12 membered heterocyclyl substituted with one or more occurrences of R^{A}. In certain embodiments, when Y is absent, A is pyrrolidinyl, piperidinyl, or 2-azaspiro[3.3]heptyl, wherein the pyrrolidinyl, piperidinyl, or 2-azaspiro[3.3]heptyl are substituted with one or more occurrences of R^{A}.

In certain embodiments, when Y is absent, A is pyrrolidinyl substituted with one or more occurrences of R^{A}. In certain embodiments, when Y is absent, A is pyrrolidinyl substituted with one occurrence of R^{A}. In certain embodiments, when Y is absent, A is pyrrolidinyl substituted with one occurrence of —(C₀₋₆ alkylene)-C(═O)OR^{A1}. In certain embodiments, when Y is absent, A is pyrrolidinyl substituted with —(C₀₋₆ alkylene)-C(═O)OR^{A1}. In certain embodiments, when Y is absent, A is pyrrolidinyl substituted with —C(═O)OH. In certain embodiments, when Y is absent, A is pyrrolidinyl substituted with —CH(CH₃)C(═O)OH. In certain embodiments, when Y is absent, A is pyrrolidinyl substituted with —C(CH₃)₂C(═O)OH.

In certain embodiments, when Y is absent, A is piperidinyl substituted with one or more occurrences of R^{A}. In certain embodiments, when Y is absent, A is piperidinyl substituted with one occurrence of R^{A}. In certain embodiments, when Y is absent, A is piperidinyl substituted with one occurrence of —(C₀₋₆ alkylene)-C(═O)OR^{A1}. In certain embodiments, when Y is absent, A is piperidinyl substituted with —(C₀₋₆, alkylene)-C(═O)OR^{A1}. In certain embodiments, when Y is absent, A is piperidinyl substituted with —C(═O)OH.

In certain embodiments, when Y is absent, A is 7-membered heterocyclyl substituted with one or more occurrences of R^{A}. In certain embodiments, when Y is absent, A is 7-membered heterocyclyl substituted with one occurrence of R^{A}. In certain embodiments, when Y is absent, A is 7-membered heterocyclyl substituted with one occurrence of —(C₀₋₆ alkylene)-C(═O)OR^{A1}. In certain embodiments, when Y is absent, A is 7-membered heterocyclyl substituted with —(C₀₋₆ alkylene)-C(═O)OR^{A1}. In certain embodiments, when Y is absent, A is 7-membered heterocyclyl substituted with —C(═O)OH.

In certain embodiments, when Y is absent, A is 2-azaspiro[3.3]heptyl substituted with one or more occurrences of R^{A} In certain embodiments, when Y is absent, A is 2-azaspiro[3.3]heptyl substituted with one occurrence of R^{A}. In certain embodiments, when Y is absent, A is 2-azaspiro[3.3]heptyl substituted with one occurrence of —(C₀₋₆ alkylene)-C(═O)OR^{A1}. In certain embodiments, when Y is absent, A is 2-azaspiro[3.3]heptyl substituted with —(C₀₋₆ alkylene)-C(═O)OR^{A1}. In certain embodiments, when Y is absent, A is 2-azaspiro[3.3]heptyl substituted with —C(═O)OH.

As generally defined above, R^{A} is independently for each occurrence oxo, halo, C₁₋₆ alkyl, —(C₀₋₆ alkylene)-C(═O)OR^{A1}, C(═O)N(R^{A1})₂, NHC(═O)R^{A1}, NHC(═O)OR^{A1}, OC(═O)N(R^{A1})₂, S(═O)₂OR^{A1}, or S(═O)₂N(R^{A1})₂. In certain embodiments, R^{A} is independently for each occurrence oxo, halo, C₁₋₆ alkyl, —(C₀₋₆ alkylene)-C(═O)OR^{A1}, or C(═O)N(R^{A1})₂. In certain embodiments, R^{A} is oxo, halo, C₁₋₆ alkyl, —(C₀₋₆ alkylene)-C(═O)OR^{A1}, or C(═O)N(R^{A1})₂. In certain embodiments, R^{A} is oxo, fluoro, methyl, —C(═O)OH, —CH(CH₃)C(═O)OH, —CH₂C(═O)OH, —C(CH₃)₂C(═O)OH, —C(═O)OCH₃, or C(═O)NH₂.

In certain embodiments, R^{A} is oxo. In certain embodiments, R^{A} is halo. In certain embodiments, R^{A} is fluoro. In certain embodiments, R^{A} is chloro. In certain embodiments, R^{A} is bromo. In certain embodiments, R^{A} is iodo. In certain embodiments, R^{A} is C₁₋₆ alkyl. In certain embodiments, R^{A} is methyl. In certain embodiments, R^{A} is —(C₀₋₆ alkylene)-C(═O)OR^{A1}. In certain embodiments, R^{A} is —C(═O)OH. In certain embodiments, R^{A} is —CH(CH₃)C(═O)OH. In certain embodiments, R^{A} is —CH₂C(═O)OH. In certain embodiments, R^{A} is —C(CH₃)₂C(═O)OH. In certain embodiments, R^{A} is —C(═O)OCH₃. In certain embodiments, R^{A} is C(═O)N(R^{A1})₂. In certain embodiments, R^{A} is —C(═O)NH₂. In certain embodiments, R^{A} is NHC(═O)R^{A}. In certain embodiments, R^{A} is NHC(═O)OR^{A1}. In certain embodiments, R^{A} is OC(═O)N(R^{A1})₂. In certain embodiments, R^{A} is S(═O)₂OR^{A1}. In certain embodiments, R^{A} is S(═O)₂N(R^{A1})₂.

As generally defined above, R^{A1} is independently for each occurrence hydrogen or C₁₋₆ alkyl. In certain embodiments, R^{A1} is hydrogen or methyl. In certain embodiments, R^{A1} is hydrogen. In certain embodiments, R^{A1} is C₁₋₆ alkyl. In certain embodiments, R^{A1} is methyl.

In certain embodiments, the invention provides a compound represented by Formula IIa:

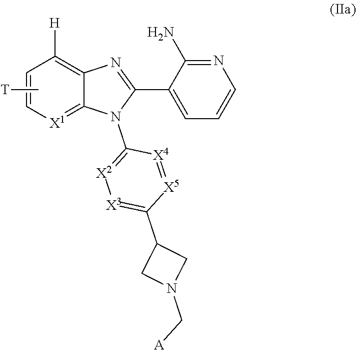

or a stereoisomer or pharmaceutically acceptable salt thereof, wherein T, X¹, X², X³, X⁴, X⁵, and A are as defined herein.

In certain embodiments, the invention provides a compound represented by Formula IIb:

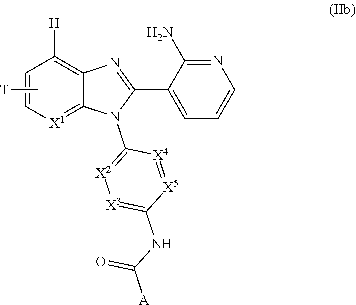

or a stereoisomer or pharmaceutically acceptable salt thereof, wherein T, X¹, X², X³, X⁴, X⁵, and A are as defined herein.

In certain embodiments, the invention provides a compound represented by Formula IIc:

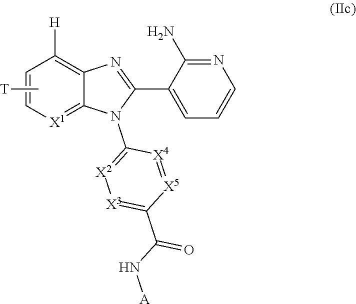

or a stereoisomer or pharmaceutically acceptable salt thereof, wherein T, X¹, X², X³, X⁴, X⁵, and A are as defined herein.

In certain embodiments, the invention provides a compound represented by Formula IId:

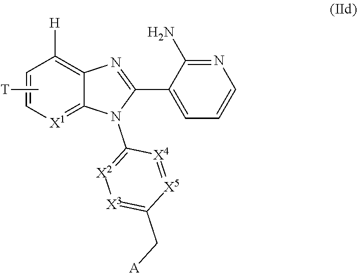

or a stereoisomer or pharmaceutically acceptable salt thereof, wherein T, X¹, X², X³, X⁴, X⁵, and A are as defined herein.

In certain embodiments, the compound is a compound of Formula IIa, IIb, IIc, or IId or a stereoisomer or pharmaceutically acceptable salt thereof:

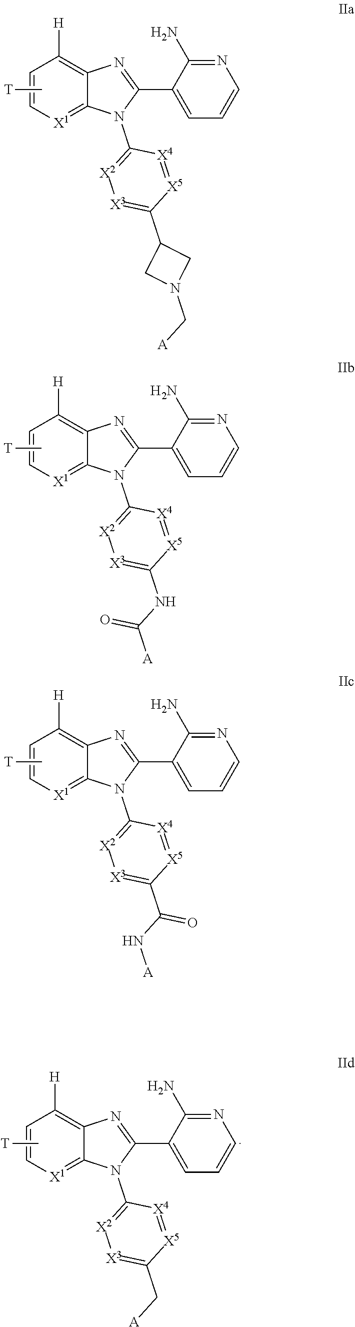

### EXEMPLARY SPECIFIC COMPOUNDS

In certain embodiments, the compound is a compound in Table 1 or a stereoisomer or pharmaceutically acceptable salt thereof. In certain embodiments, the compound is a compound in Table 1. It is contemplated herein, in certain embodiments, that any free carboxylic acid present in a compound in Table 1 could be readily replaced with a corresponding ester group (e.g. a methyl ester). It is also contemplated herein, in certain embodiments, that any ester group (e.g. a methyl ester) present in a compound in Table 1 could be readily replaced with a free carboxylic acid.

TABLE 1

| Compound No. | Chemical Structure |
|---|---|
| I-1 | 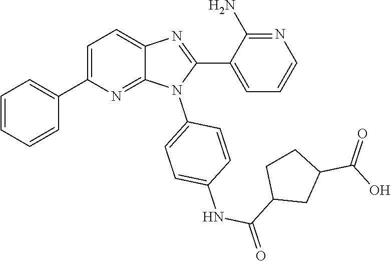 [SMILES: Nc1ncccc1-c1nc2ccc(-c3ccccc3)nc2n1-c1ccc(NC(=O)C2CCC(C(=O)O)C2)cc1] |
| I-2 | 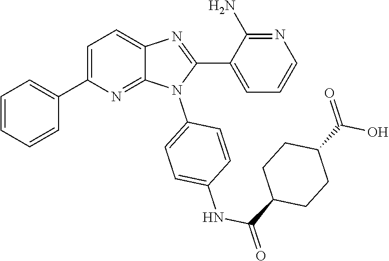 [SMILES: Nc1ncccc1-c1nc2ccc(-c3ccccc3)nc2n1-c1ccc(NC(=O)[C@H]2CC[C@H](C(=O)O)CC2)cc1] |
| I-3 | 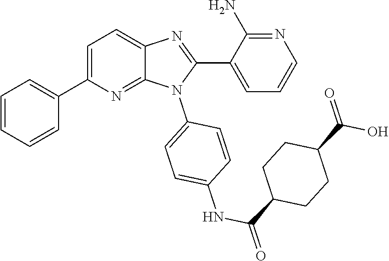 [SMILES: Nc1ncccc1-c1nc2ccc(-c3ccccc3)nc2n1-c1ccc(NC(=O)[C@H]2CC[C@@H](C(=O)O)CC2)cc1] |
| I-4 | 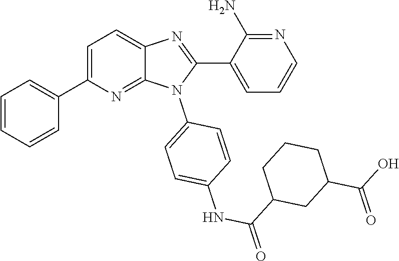 [SMILES: Nc1ncccc1-c1nc2ccc(-c3ccccc3)nc2n1-c1ccc(NC(=O)C2CCCC(C(=O)O)C2)cc1] |
| I-5 | 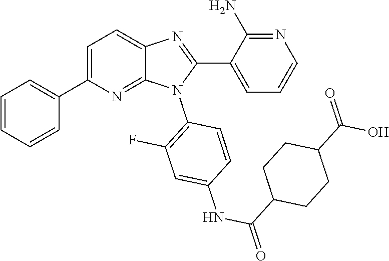 [SMILES: Nc1ncccc1-c1nc2ccc(-c3ccccc3)nc2n1-c1ccc(NC(=O)C2CCC(C(=O)O)CC2)cc1F] |
| I-6 | 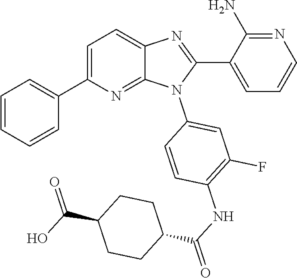 [SMILES: Nc1ncccc1-c1nc2ccc(-c3ccccc3)nc2n1-c1ccc(NC(=O)[C@H]2CC[C@H](C(=O)O)CC2)c(F)c1] |
| I-7 | 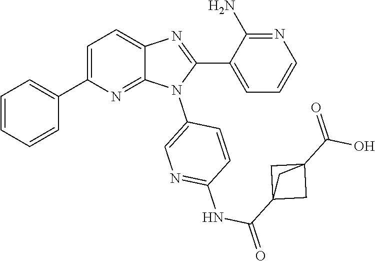 [SMILES: Nc1ncccc1-c1nc2ccc(-c3ccccc3)nc2n1-c1ccc(NC(=O)C23CC(C(=O)O)(C2)C3)nc1] |
| I-8 | 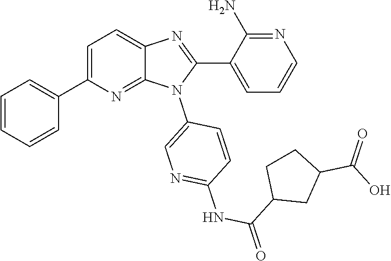 [SMILES: Nc1ncccc1-c1nc2ccc(-c3ccccc3)nc2n1-c1ccc(NC(=O)C2CCC(C(=O)O)C2)nc1] |
| I-9 | 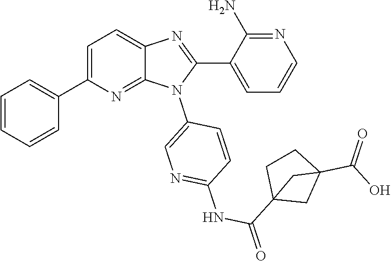 [SMILES: Nc1ncccc1-c1nc2ccc(-c3ccccc3)nc2n1-c1ccc(NC(=O)C23CCC(C(=O)O)(C2)C3)nc1] |
| I-10 | 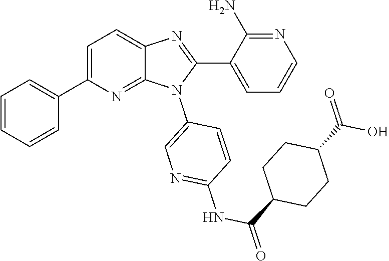 [SMILES: Nc1ncccc1-c1nc2ccc(-c3ccccc3)nc2n1-c1ccc(NC(=O)[C@H]2CC[C@H](C(=O)O)CC2)nc1] |
| I-11 | 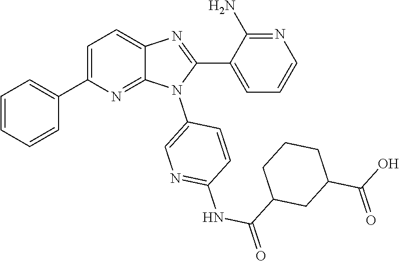 [SMILES: Nc1ncccc1-c1nc2ccc(-c3ccccc3)nc2n1-c1ccc(NC(=O)C2CCCC(C(=O)O)C2)nc1] |
| I-12 | 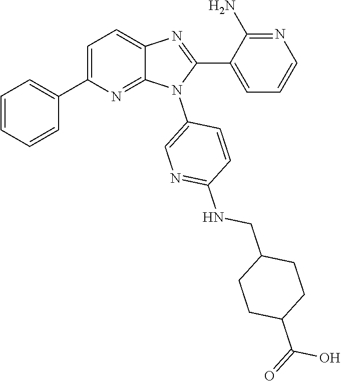 [SMILES: Nc1ncccc1-c1nc2ccc(-c3ccccc3)nc2n1-c1ccc(NCC2CCC(C(=O)O)CC2)nc1] |
| I-13 | 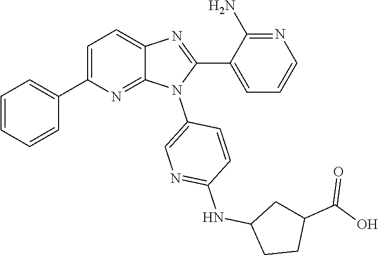 [SMILES: Nc1ncccc1-c1nc2ccc(-c3ccccc3)nc2n1-c1ccc(NC2CCC(C(=O)O)C2)nc1] |
| I-14 | 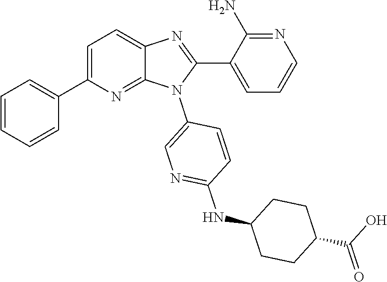 [SMILES: Nc1ncccc1-c1nc2ccc(-c3ccccc3)nc2n1-c1ccc(N[C@H]2CC[C@H](C(=O)O)CC2)nc1] |
| I-15 | 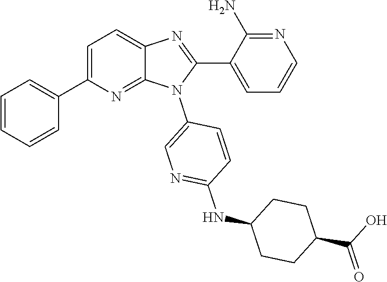 [SMILES: Nc1ncccc1-c1nc2ccc(-c3ccccc3)nc2n1-c1ccc(N[C@H]2CC[C@@H](C(=O)O)CC2)nc1] |
| I-16 | 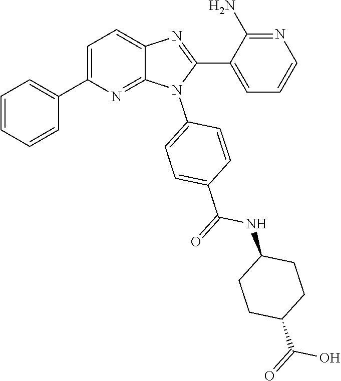 [SMILES: Nc1ncccc1-c1nc2ccc(-c3ccccc3)nc2n1-c1ccc(C(=O)N[C@H]2CC[C@H](C(=O)O)CC2)cc1] |
| I-17 | 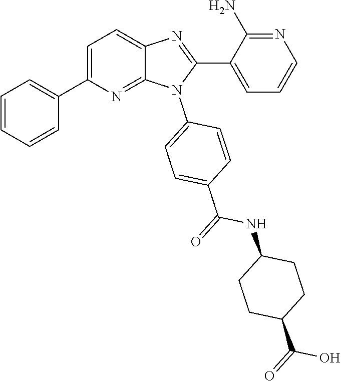 [SMILES: Nc1ncccc1-c1nc2ccc(-c3ccccc3)nc2n1-c1ccc(C(=O)N[C@H]2CC[C@@H](C(=O)O)CC2)cc1] |
| I-18 | 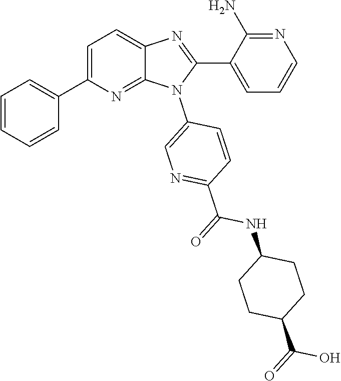 [SMILES: Nc1ncccc1-c1nc2ccc(-c3ccccc3)nc2n1-c1ccc(C(=O)N[C@H]2CC[C@@H](C(=O)O)CC2)nc1] |
| I-19 | 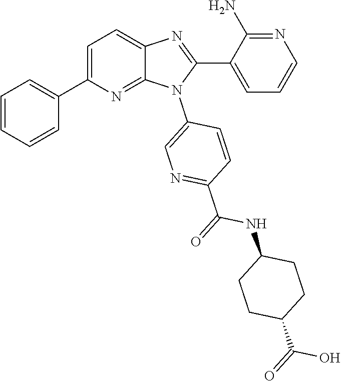 [SMILES: Nc1ncccc1-c1nc2ccc(-c3ccccc3)nc2n1-c1ccc(C(=O)N[C@H]2CC[C@H](C(=O)O)CC2)nc1] |
| I-20 | 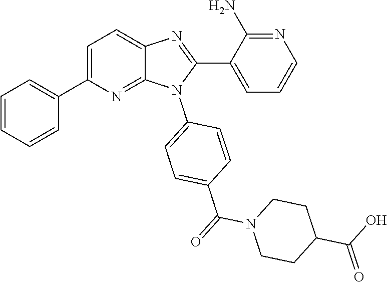 [SMILES: Nc1ncccc1-c1nc2ccc(-c3ccccc3)nc2n1-c1ccc(C(=O)N2CCC(C(=O)O)CC2)cc1] |
| I-21 | 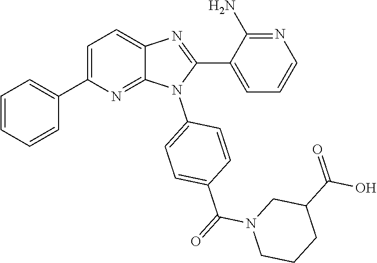 [SMILES: Nc1ncccc1-c1nc2ccc(-c3ccccc3)nc2n1-c1ccc(C(=O)N2CCCC(C(=O)O)C2)cc1] |
| I-22 | 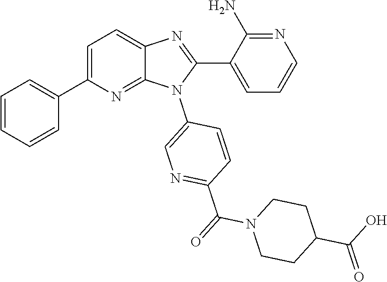 [SMILES: Nc1ncccc1-c1nc2ccc(-c3ccccc3)nc2n1-c1ccc(C(=O)N2CCC(C(=O)O)CC2)nc1] |
| I-23 | 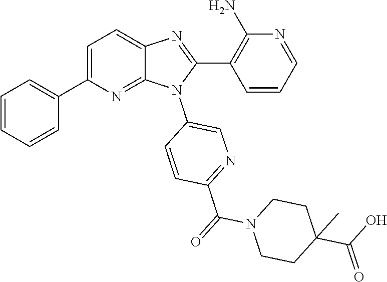 [SMILES: CC1(C(=O)O)CCN(C(=O)c2ccc(-n3c(-c4cccnc4N)nc4ccc(-c5ccccc5)nc43)cn2)CC1] |
| I-24 | 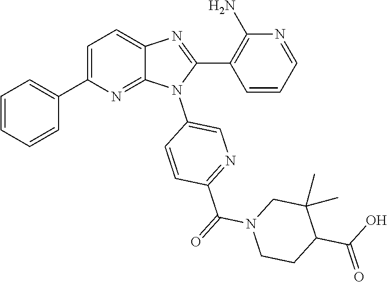 [SMILES: CC1(C)CN(C(=O)c2ccc(-n3c(-c4cccnc4N)nc4ccc(-c5ccccc5)nc43)cn2)CCC1C(=O)O] |
| I-25 | 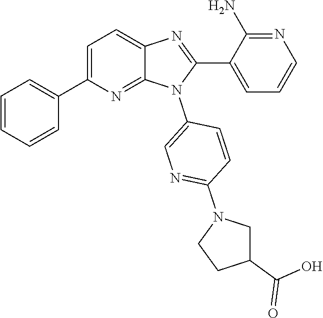 [SMILES: Nc1ncccc1-c1nc2ccc(-c3ccccc3)nc2n1-c1ccc(N2CCC(C(=O)O)C2)nc1] |
| I-26 | 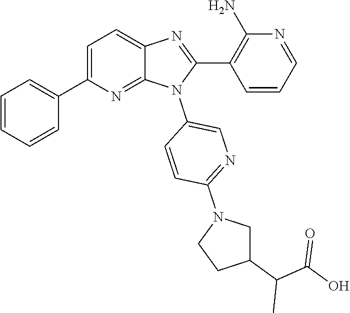 [SMILES: CC(C(=O)O)C1CCN(c2ccc(-n3c(-c4cccnc4N)nc4ccc(-c5ccccc5)nc43)cn2)C1] |
| I-27 | 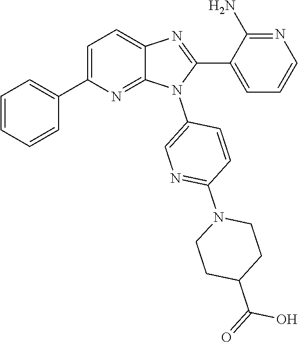 [SMILES: Nc1ncccc1-c1nc2ccc(-c3ccccc3)nc2n1-c1ccc(N2CCC(C(=O)O)CC2)nc1] |
| I-28 | 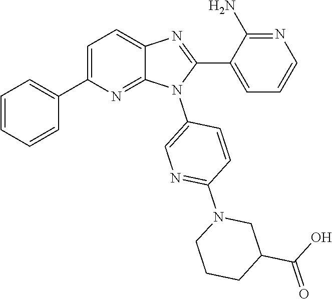 [SMILES: Nc1ncccc1-c1nc2ccc(-c3ccccc3)nc2n1-c1ccc(N2CCCC(C(=O)O)C2)nc1] |
| I-29 | 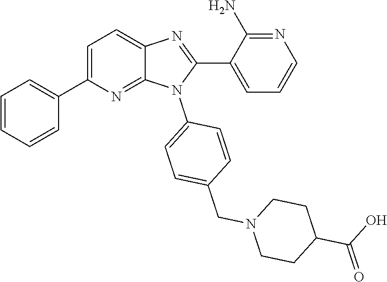 [SMILES: Nc1ncccc1-c1nc2ccc(-c3ccccc3)nc2n1-c1ccc(CN2CCC(C(=O)O)CC2)cc1] |
| I-30 | 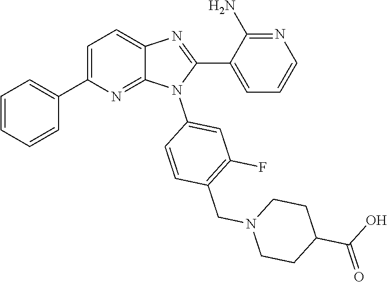 [SMILES: Nc1ncccc1-c1nc2ccc(-c3ccccc3)nc2n1-c1ccc(CN2CCC(C(=O)O)CC2)c(F)c1] |
| I-31 | 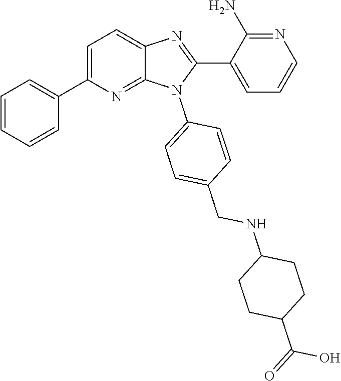 [SMILES: Nc1ncccc1-c1nc2ccc(-c3ccccc3)nc2n1-c1ccc(CNC2CCC(C(=O)O)CC2)cc1] |
| I-32 | 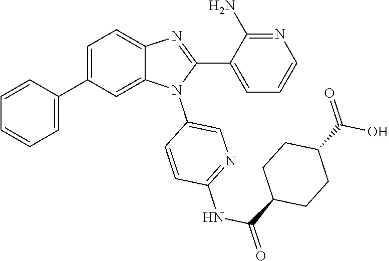 [SMILES: Nc1ncccc1-c1nc2ccc(-c3ccccc3)cc2n1-c1ccc(NC(=O)[C@H]2CC[C@H](C(=O)O)CC2)nc1] |
| I-33 | 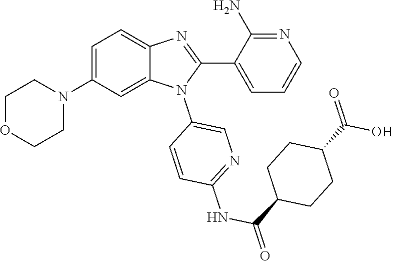 [SMILES: Nc1ncccc1-c1nc2ccc(N3CCOCC3)cc2n1-c1ccc(NC(=O)[C@H]2CC[C@H](C(=O)O)CC2)nc1] |
| I-34 | 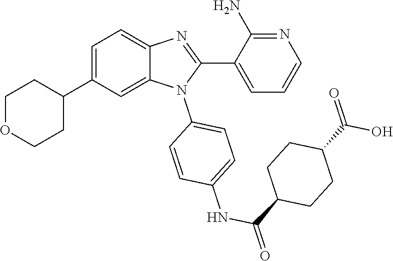 [SMILES: Nc1ncccc1-c1nc2ccc(C3CCOCC3)cc2n1-c1ccc(NC(=O)[C@H]2CC[C@H](C(=O)O)CC2)cc1] |
| I-35 | 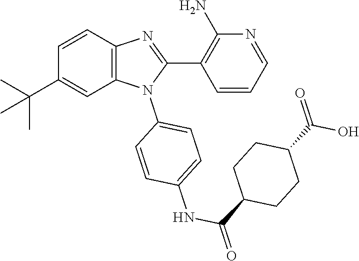 [SMILES: CC(C)(C)c1ccc2nc(-c3cccnc3N)n(-c3ccc(NC(=O)[C@H]4CC[C@H](C(=O)O)CC4)cc3)c2c1] |
| I-36 | 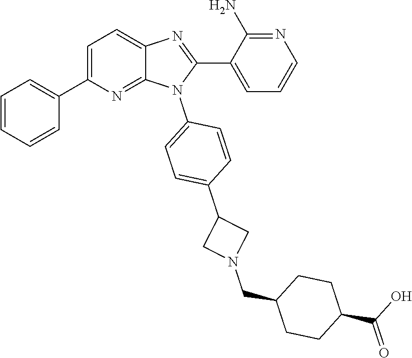 [SMILES: Nc1ncccc1-c1nc2ccc(-c3ccccc3)nc2n1-c1ccc(C2CN(C[C@H]3CC[C@@H](C(=O)O)CC3)C2)cc1] |
| I-37 | 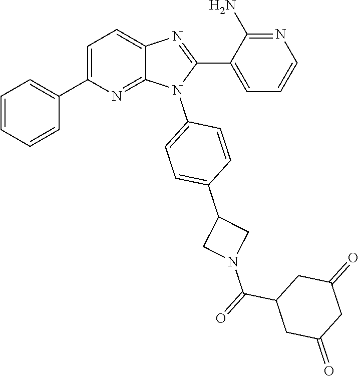 [SMILES: Nc1ncccc1-c1nc2ccc(-c3ccccc3)nc2n1-c1ccc(C2CN(C(=O)C3CC(=O)CC(=O)C3)C2)cc1] |
| I-38 | 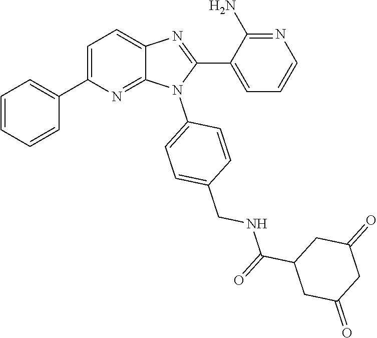 [SMILES: Nc1ncccc1-c1nc2ccc(-c3ccccc3)nc2n1-c1ccc(CNC(=O)C2CC(=O)CC(=O)C2)cc1] |
| I-39 | 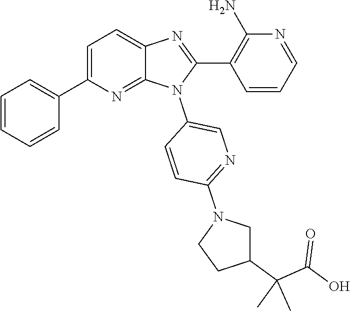 [SMILES: CC(C)(C(=O)O)C1CCN(c2ccc(-n3c(-c4cccnc4N)nc4ccc(-c5ccccc5)nc43)cn2)C1] |
| I-40 | 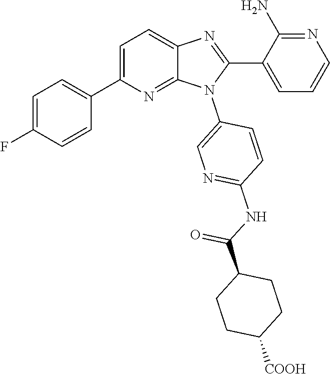 [SMILES: Nc1ncccc1-c1nc2ccc(-c3ccc(F)cc3)nc2n1-c1ccc(NC(=O)[C@H]2CC[C@H](C(=O)O)CC2)nc1] |
| I-41 | 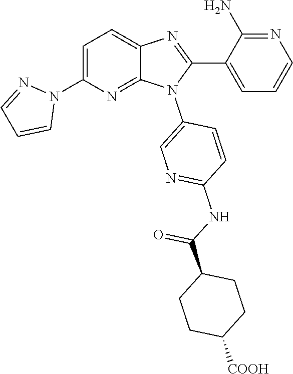 [SMILES: Nc1ncccc1-c1nc2ccc(-n3cccn3)nc2n1-c1ccc(NC(=O)[C@H]2CC[C@H](C(=O)O)CC2)nc1] |
| I-42 | 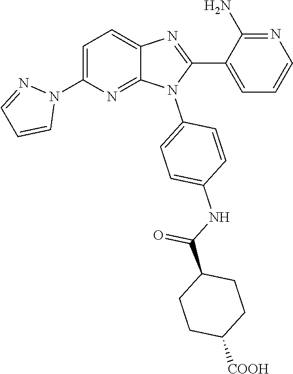 [SMILES: Nc1ncccc1-c1nc2ccc(-n3cccn3)nc2n1-c1ccc(NC(=O)[C@H]2CC[C@H](C(=O)O)CC2)cc1] |
| I-43 | 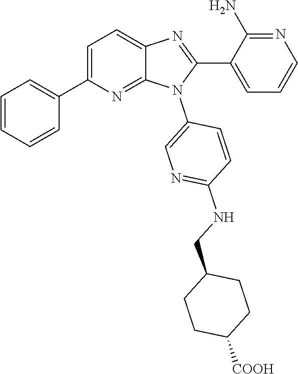 [SMILES: Nc1ncccc1-c1nc2ccc(-c3ccccc3)nc2n1-c1ccc(NC[C@H]2CC[C@H](C(=O)O)CC2)nc1] |
| I-44 | 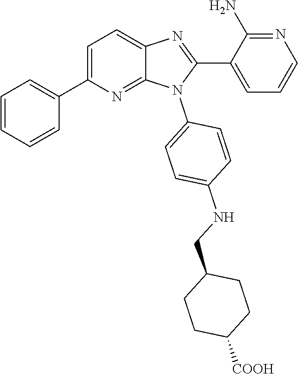 [SMILES: Nc1ncccc1-c1nc2ccc(-c3ccccc3)nc2n1-c1ccc(NC[C@H]2CC[C@H](C(=O)O)CC2)cc1] |
| I-45 | 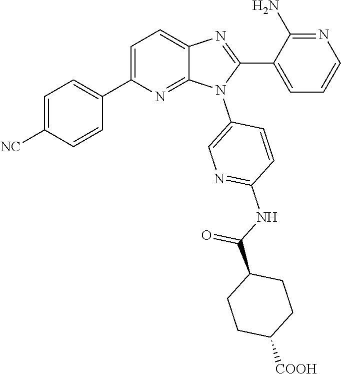 [SMILES: N#Cc1ccc(-c2ccc3nc(-c4cccnc4N)n(-c4ccc(NC(=O)[C@H]5CC[C@H](C(=O)O)CC5)nc4)c3n2)cc1] |
| I-46 | 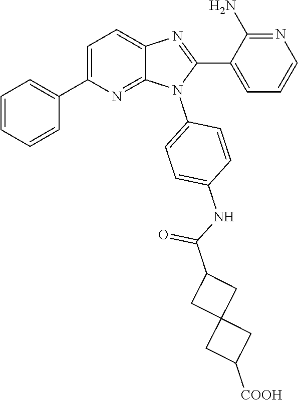 [SMILES: Nc1ncccc1-c1nc2ccc(-c3ccccc3)nc2n1-c1ccc(NC(=O)C2CC3(CC(C(=O)O)C3)C2)cc1] |
| I-47 | 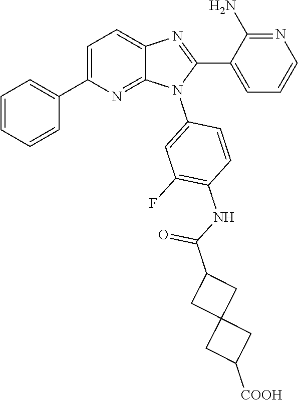 [SMILES: Nc1ncccc1-c1nc2ccc(-c3ccccc3)nc2n1-c1ccc(NC(=O)C2CC3(CC(C(=O)O)C3)C2)c(F)c1] |
| I-48 | 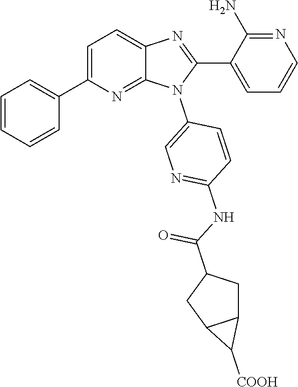 [SMILES: Nc1ncccc1-c1nc2ccc(-c3ccccc3)nc2n1-c1ccc(NC(=O)C2CC3C(C2)C3C(=O)O)nc1] |
| I-49 | 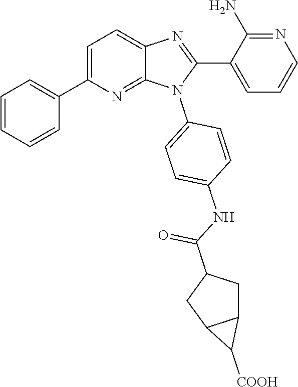 [SMILES: Nc1ncccc1-c1nc2ccc(-c3ccccc3)nc2n1-c1ccc(NC(=O)C2CC3C(C2)C3C(=O)O)cc1] |
| I-50 | 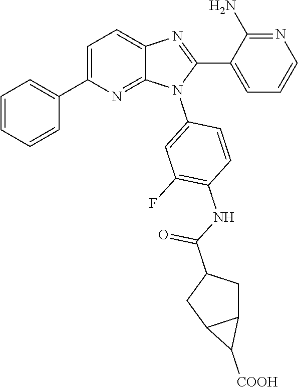 [SMILES: Nc1ncccc1-c1nc2ccc(-c3ccccc3)nc2n1-c1ccc(NC(=O)C2CC3C(C2)C3C(=O)O)c(F)c1] |
| I-51 | 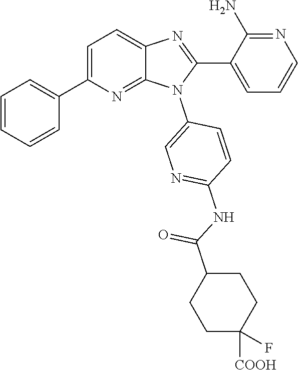 [SMILES: Nc1ncccc1-c1nc2ccc(-c3ccccc3)nc2n1-c1ccc(NC(=O)C2CCC(F)(C(=O)O)CC2)nc1] |
| I-52 | 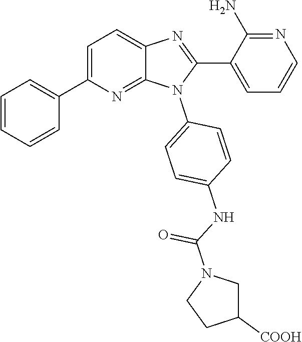 [SMILES: Nc1ncccc1-c1nc2ccc(-c3ccccc3)nc2n1-c1ccc(NC(=O)N2CCC(C(=O)O)C2)cc1] |
| 1-53 | 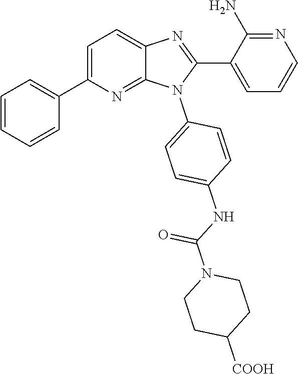 [SMILES: Nc1ncccc1-c1nc2ccc(-c3ccccc3)nc2n1-c1ccc(NC(=O)N2CCC(C(=O)O)CC2)cc1] |
| I-54 | 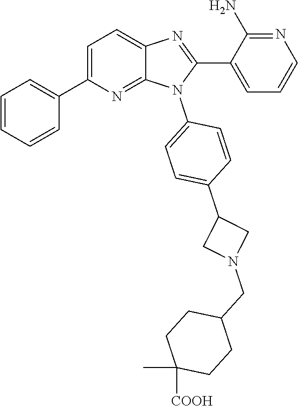 [SMILES: CC1(C(=O)O)CCC(CN2CC(c3ccc(-n4c(-c5cccnc5N)nc5ccc(-c6ccccc6)nc54)cc3)C2)CC1] |
| I-55 | 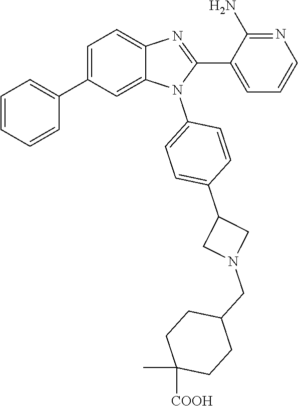 [SMILES: CC1(C(=O)O)CCC(CN2CC(c3ccc(-n4c(-c5cccnc5N)nc5ccc(-c6ccccc6)cc54)cc3)C2)CC1] |
| I-56 | 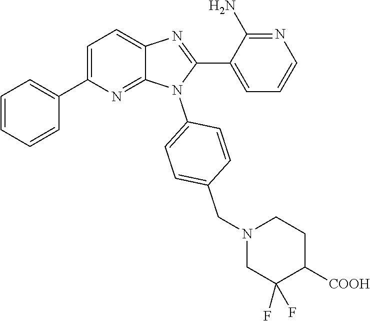 [SMILES: Nc1ncccc1-c1nc2ccc(-c3ccccc3)nc2n1-c1ccc(CN2CCC(C(=O)O)C(F)(F)C2)cc1] |
| I-57 | 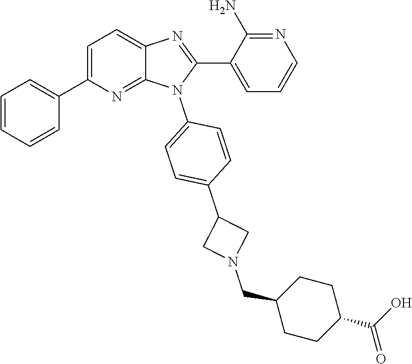 [SMILES: Nc1ncccc1-c1nc2ccc(-c3ccccc3)nc2n1-c1ccc(C2CN(C[C@H]3CC[C@H](C(=O)O)CC3)C2)cc1] |
| I-58 | 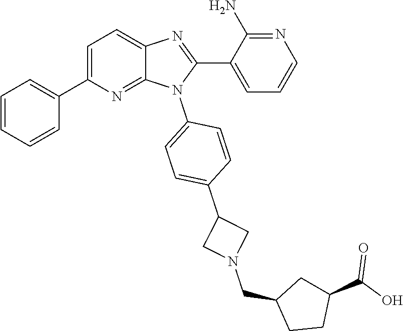 [SMILES: Nc1ncccc1-c1nc2ccc(-c3ccccc3)nc2n1-c1ccc(C2CN(C[C@@H]3CC[C@H](C(=O)O)C3)C2)cc1] |
| I-59 | 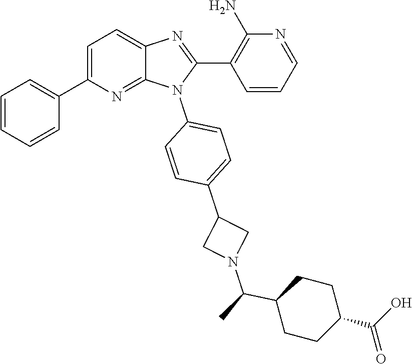 [SMILES: C[C@H]([C@H]1CC[C@H](C(=O)O)CC1)N1CC(c2ccc(-n3c(-c4cccnc4N)nc4ccc(-c5ccccc5)nc43)cc2)C1] |
| I-60 | 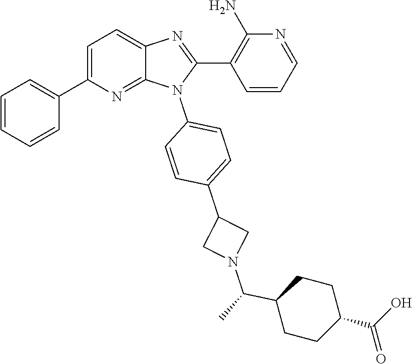 [SMILES: C[C@@H]([C@H]1CC[C@H](C(=O)O)CC1)N1CC(c2ccc(-n3c(-c4cccnc4N)nc4ccc(-c5ccccc5)nc43)cc2)C1] |
| I-61 | 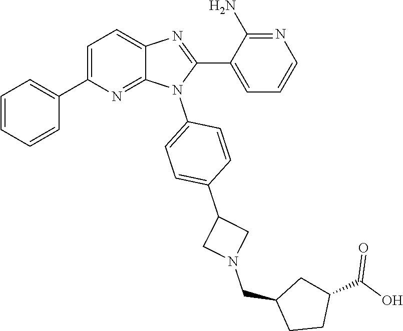 [SMILES: Nc1ncccc1-c1nc2ccc(-c3ccccc3)nc2n1-c1ccc(C2CN(C[C@@H]3CC[C@@H](C(=O)O)C3)C2)cc1] |
| I-62 | 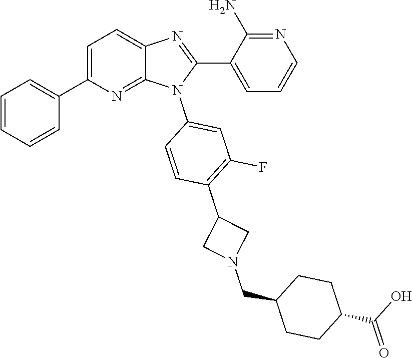 [SMILES: Nc1ncccc1-c1nc2ccc(-c3ccccc3)nc2n1-c1ccc(C2CN(C[C@H]3CC[C@H](C(=O)O)CC3)C2)c(F)c1] |
| I-63 | 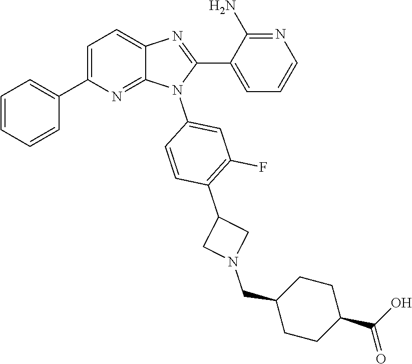 [SMILES: Nc1ncccc1-c1nc2ccc(-c3ccccc3)nc2n1-c1ccc(C2CN(C[C@H]3CC[C@@H](C(=O)O)CC3)C2)c(F)c1] |
| I-64 | 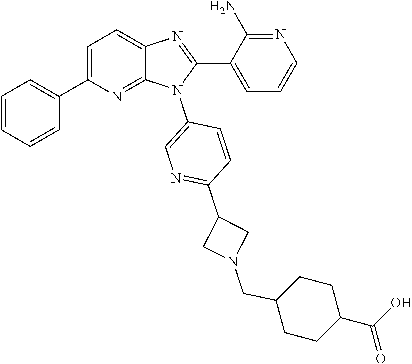 [SMILES: Nc1ncccc1-c1nc2ccc(-c3ccccc3)nc2n1-c1ccc(C2CN(CC3CCC(C(=O)O)CC3)C2)nc1] |
| I-65 | 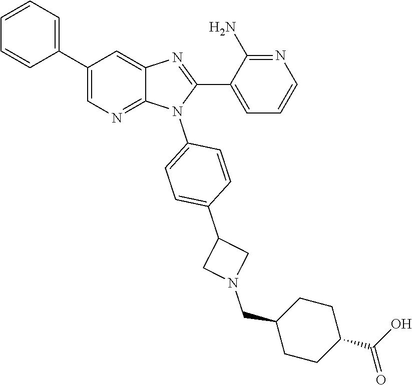 [SMILES: Nc1ncccc1-c1nc2cc(-c3ccccc3)cnc2n1-c1ccc(C2CN(C[C@H]3CC[C@H](C(=O)O)CC3)C2)cc1] |
| I-66 | 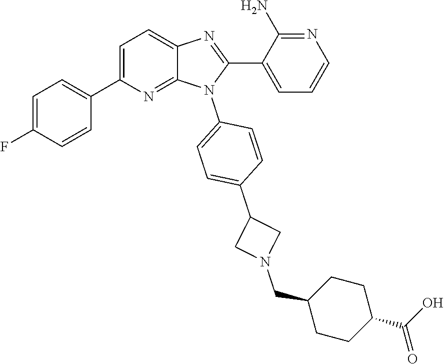 [SMILES: Nc1ncccc1-c1nc2ccc(-c3ccc(F)cc3)nc2n1-c1ccc(C2CN(C[C@H]3CC[C@H](C(=O)O)CC3)C2)cc1] |
| I-67 | 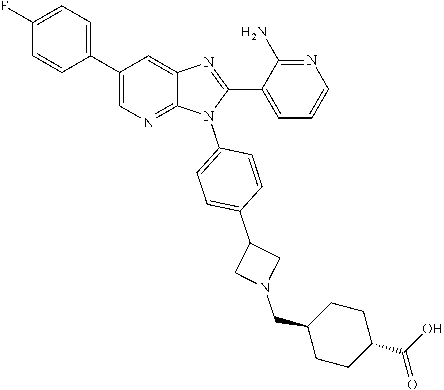 [SMILES: Nc1ncccc1-c1nc2cc(-c3ccc(F)cc3)cnc2n1-c1ccc(C2CN(C[C@H]3CC[C@H](C(=O)O)CC3)C2)cc1] |
| I-68 | 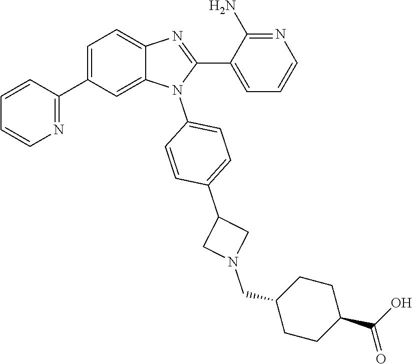 [SMILES: Nc1ncccc1-c1nc2ccc(-c3ccccn3)cc2n1-c1ccc(C2CN(C[C@H]3CC[C@H](C(=O)O)CC3)C2)cc1] |
| I-69 | 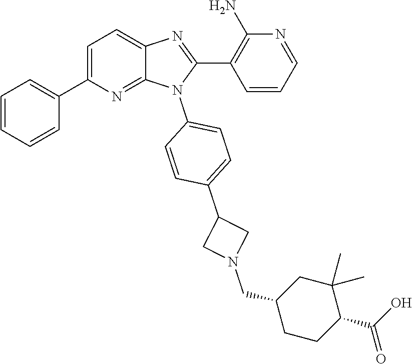 [SMILES: CC1(C)C[C@@H](CN2CC(c3ccc(-n4c(-c5cccnc5N)nc5ccc(-c6ccccc6)nc54)cc3)C2)CC[C@H]1C(=O)O] |
| I-70 | 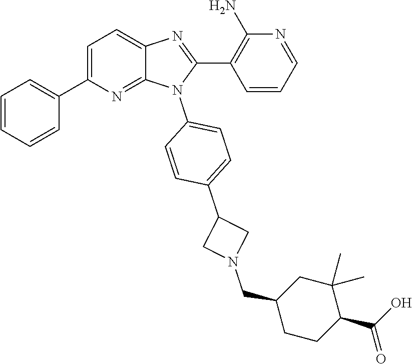 [SMILES: CC1(C)C[C@H](CN2CC(c3ccc(-n4c(-c5cccnc5N)nc5ccc(-c6ccccc6)nc54)cc3)C2)CC[C@@H]1C(=O)O] |
| I-71 | 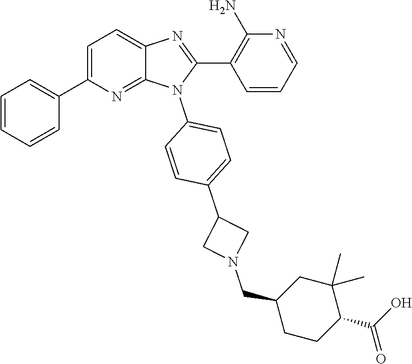 [SMILES: CC1(C)C[C@H](CN2CC(c3ccc(-n4c(-c5cccnc5N)nc5ccc(-c6ccccc6)nc54)cc3)C2)CC[C@H]1C(=O)O] |
| I-72 | 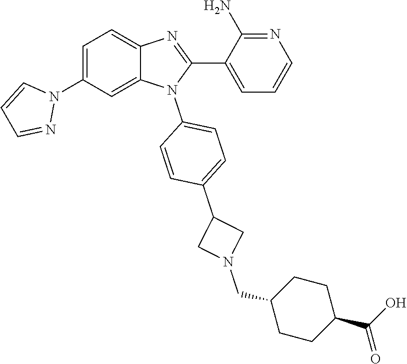 [SMILES: Nc1ncccc1-c1nc2ccc(-n3cccn3)cc2n1-c1ccc(C2CN(C[C@H]3CC[C@H](C(=O)O)CC3)C2)cc1] |
| I-73 | 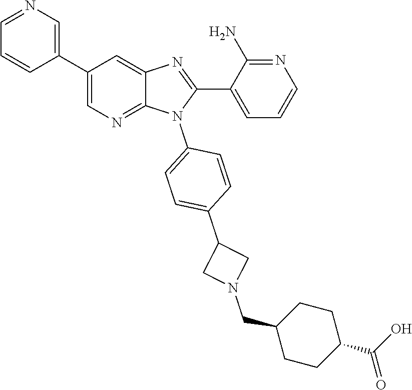 [SMILES: Nc1ncccc1-c1nc2cc(-c3cccnc3)cnc2n1-c1ccc(C2CN(C[C@H]3CC[C@H](C(=O)O)CC3)C2)cc1] |
| I-74 | 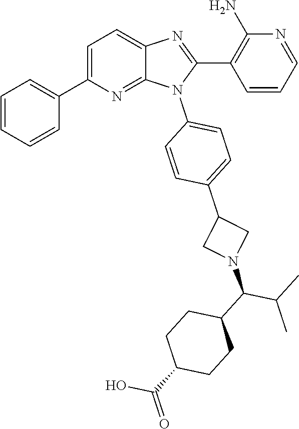 [SMILES: CC(C)[C@@H]([C@H]1CC[C@H](C(=O)O)CC1)N1CC(c2ccc(-n3c(-c4cccnc4N)nc4ccc(-c5ccccc5)nc43)cc2)C1] |
| I-75 | 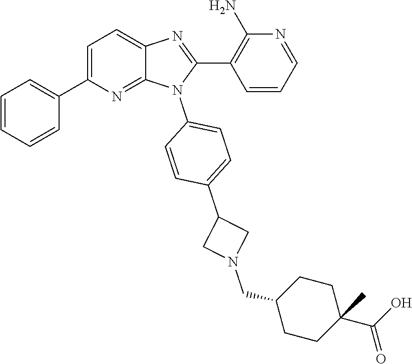 [SMILES: C[C@]1(C(=O)O)CC[C@@H](CN2CC(c3ccc(-n4c(-c5cccnc5N)nc5ccc(-c6ccccc6)nc54)cc3)C2)CC1] |
| I-76 | 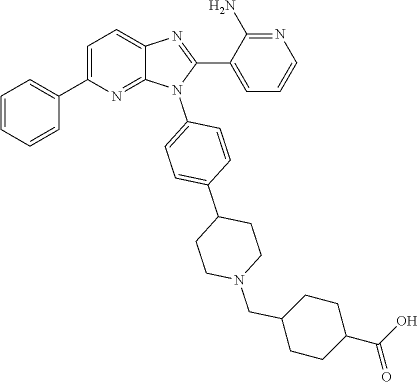 [SMILES: Nc1ncccc1-c1nc2ccc(-c3ccccc3)nc2n1-c1ccc(C2CCN(CC3CCC(C(=O)O)CC3)CC2)cc1] |
| I-77 | 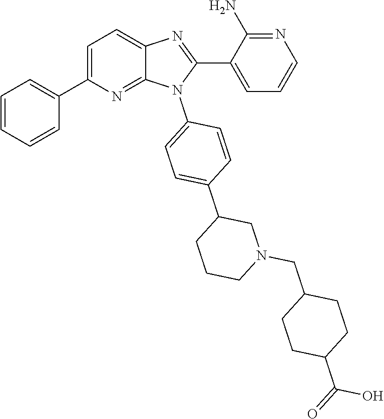 [SMILES: Nc1ncccc1-c1nc2ccc(-c3ccccc3)nc2n1-c1ccc(C2CCCN(CC3CCC(C(=O)O)CC3)C2)cc1] |
| I-78 | 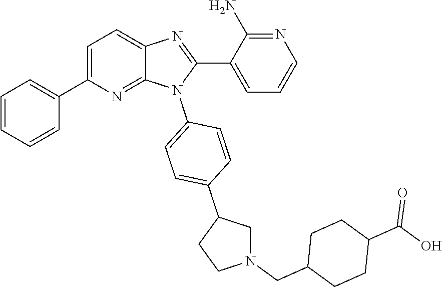 [SMILES: Nc1ncccc1-c1nc2ccc(-c3ccccc3)nc2n1-c1ccc(C2CCN(CC3CCC(C(=O)O)CC3)C2)cc1] |
| I-79 | 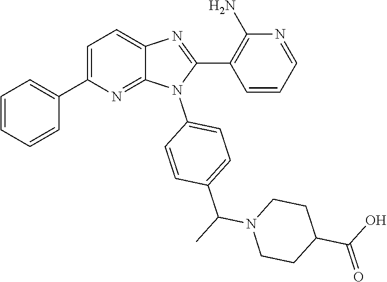 [SMILES: CC(c1ccc(-n2c(-c3cccnc3N)nc3ccc(-c4ccccc4)nc32)cc1)N1CCC(C(=O)O)CC1] |
| I-80 | 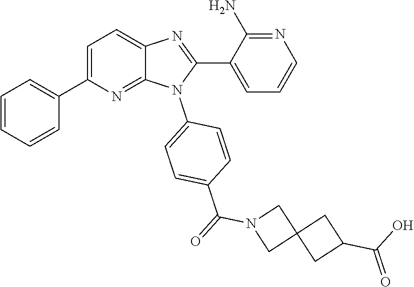 [SMILES: Nc1ncccc1-c1nc2ccc(-c3ccccc3)nc2n1-c1ccc(C(=O)N2CC3(CC(C(=O)O)C3)C2)cc1] |
| I-81 | 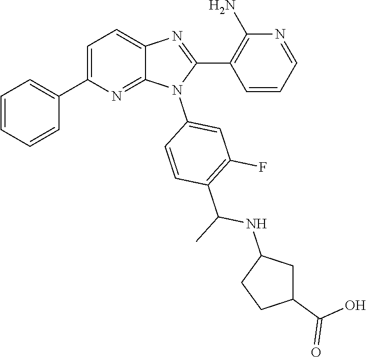 [SMILES: CC(NC1CCC(C(=O)O)C1)c1ccc(-n2c(-c3cccnc3N)nc3ccc(-c4ccccc4)nc32)cc1F] |
| I-82 | 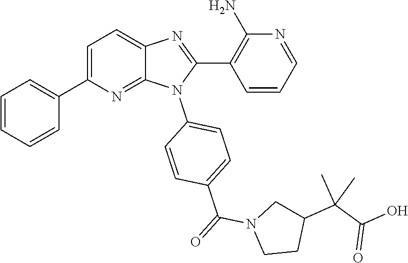 [SMILES: CC(C)(C(=O)O)C1CCN(C(=O)c2ccc(-n3c(-c4cccnc4N)nc4ccc(-c5ccccc5)nc43)cc2)C1] |
| I-83 | 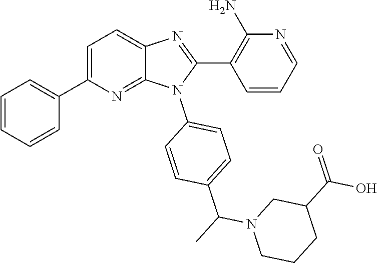 [SMILES: CC(c1ccc(-n2c(-c3cccnc3N)nc3ccc(-c4ccccc4)nc32)cc1)N1CCCC(C(=O)O)C1] |
| I-84 | 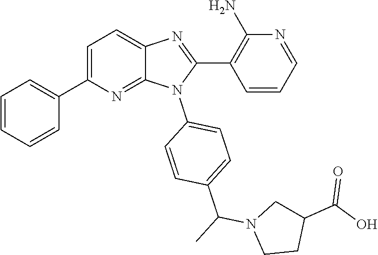 [SMILES: CC(c1ccc(-n2c(-c3cccnc3N)nc3ccc(-c4ccccc4)nc32)cc1)N1CCC(C(=O)O)C1] |
| I-85 | 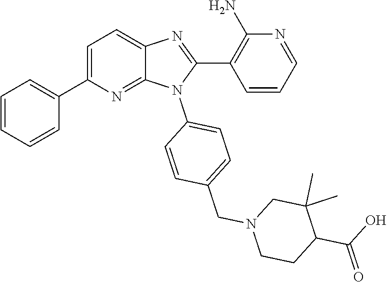 [SMILES: CC1(C)CN(Cc2ccc(-n3c(-c4cccnc4N)nc4ccc(-c5ccccc5)nc43)cc2)CCC1C(=O)O] |
| I-86 | 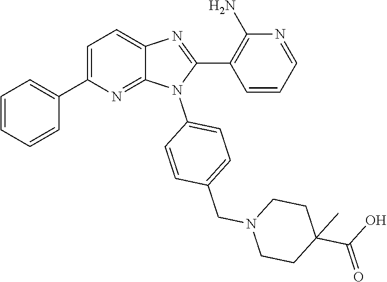 [SMILES: CC1(C(=O)O)CCN(Cc2ccc(-n3c(-c4cccnc4N)nc4ccc(-c5ccccc5)nc43)cc2)CC1] |
| I-87 | 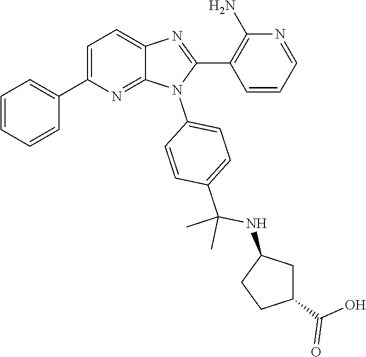 [SMILES: CC(C)(N[C@@H]1CC[C@@H](C(=O)O)C1)c1ccc(-n2c(-c3cccnc3N)nc3ccc(-c4ccccc4)nc32)cc1] |
| I-88 | 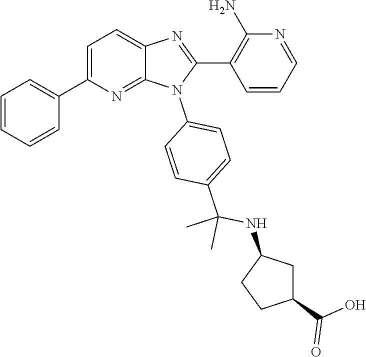 [SMILES: CC(C)(N[C@@H]1CC[C@H](C(=O)O)C1)c1ccc(-n2c(-c3cccnc3N)nc3ccc(-c4ccccc4)nc32)cc1] |
| I-89 | 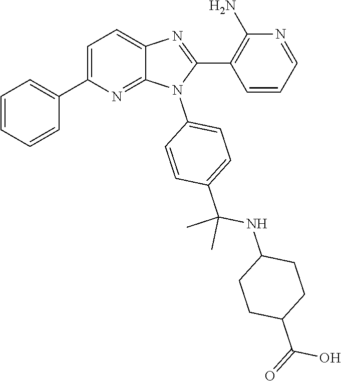 [SMILES: CC(C)(NC1CCC(C(=O)O)CC1)c1ccc(-n2c(-c3cccnc3N)nc3ccc(-c4ccccc4)nc32)cc1] |
| I-90 | 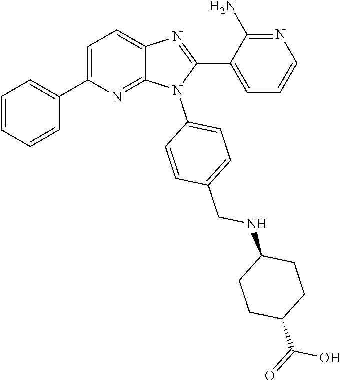 [SMILES: Nc1ncccc1-c1nc2ccc(-c3ccccc3)nc2n1-c1ccc(CN[C@H]2CC[C@H](C(=O)O)CC2)cc1] |
| I-91 | 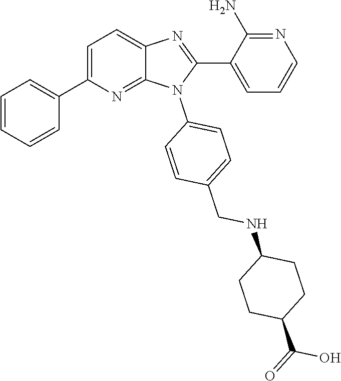 [SMILES: Nc1ncccc1-c1nc2ccc(-c3ccccc3)nc2n1-c1ccc(CN[C@H]2CC[C@@H](C(=O)O)CC2)cc1] |
| I-92 | 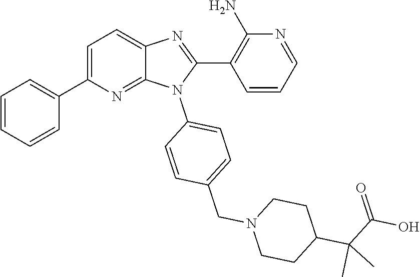 [SMILES: CC(C)(C(=O)O)C1CCN(Cc2ccc(-n3c(-c4cccnc4N)nc4ccc(-c5ccccc5)nc43)cc2)CC1] |
| I-93 | 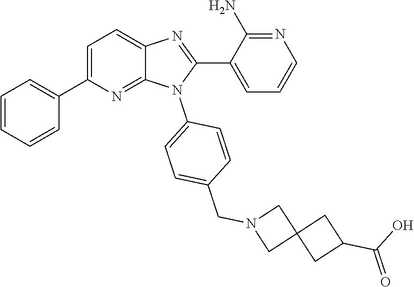 [SMILES: Nc1ncccc1-c1nc2ccc(-c3ccccc3)nc2n1-c1ccc(CN2CC3(CC(C(=O)O)C3)C2)cc1] |
| I-94 | 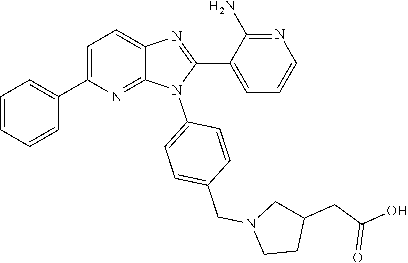 [SMILES: Nc1ncccc1-c1nc2ccc(-c3ccccc3)nc2n1-c1ccc(CN2CCC(CC(=O)O)C2)cc1] |
| I-95 | 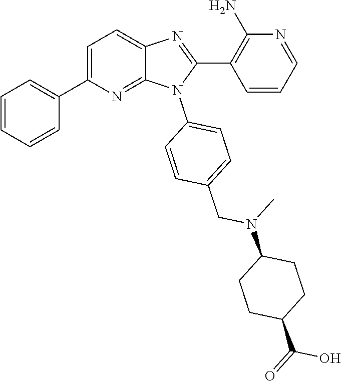 [SMILES: CN(Cc1ccc(-n2c(-c3cccnc3N)nc3ccc(-c4ccccc4)nc32)cc1)[C@H]1CC[C@@H](C(=O)O)CC1] |
| I-96 | 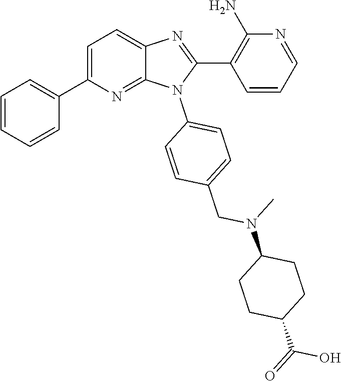 [SMILES: CN(Cc1ccc(-n2c(-c3cccnc3N)nc3ccc(-c4ccccc4)nc32)cc1)[C@H]1CC[C@H](C(=O)O)CC1] |
| I-97 | 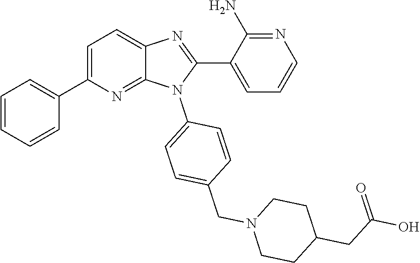 [SMILES: Nc1ncccc1-c1nc2ccc(-c3ccccc3)nc2n1-c1ccc(CN2CCC(CC(=O)O)CC2)cc1] |
| I-98 | 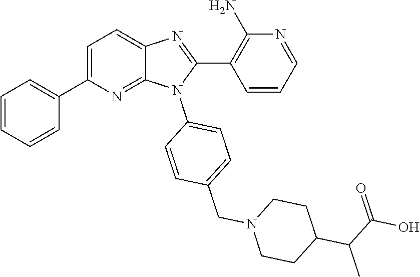 [SMILES: CC(C(=O)O)C1CCN(Cc2ccc(-n3c(-c4cccnc4N)nc4ccc(-c5ccccc5)nc43)cc2)CC1] |
| I-99 | 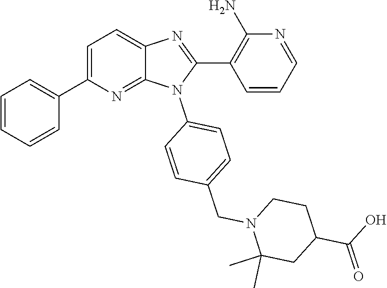 [SMILES: CC1(C)CC(C(=O)O)CCN1Cc1ccc(-n2c(-c3cccnc3N)nc3ccc(-c4ccccc4)nc32)cc1] |
| I-100 | 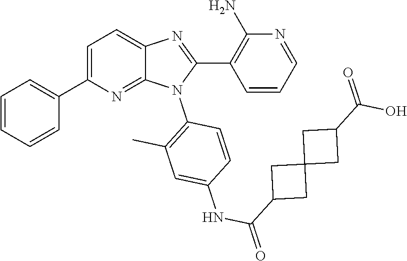 [SMILES: Cc1cc(NC(=O)C2CC3(CC(C(=O)O)C3)C2)ccc1-n1c(-c2cccnc2N)nc2ccc(-c3ccccc3)nc21] |
| I-101 | 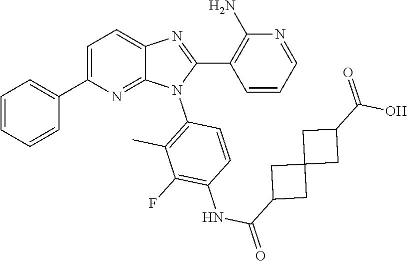 [SMILES: Cc1c(-n2c(-c3cccnc3N)nc3ccc(-c4ccccc4)nc32)ccc(NC(=O)C2CC3(CC(C(=O)O)C3)C2)c1F] |
| I-102 | 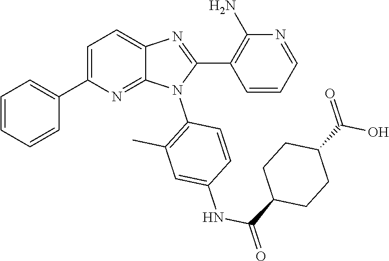 [SMILES: Cc1cc(NC(=O)[C@H]2CC[C@H](C(=O)O)CC2)ccc1-n1c(-c2cccnc2N)nc2ccc(-c3ccccc3)nc21] |
| I-103 | 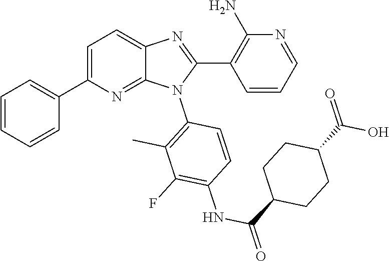 [SMILES: Cc1c(-n2c(-c3cccnc3N)nc3ccc(-c4ccccc4)nc32)ccc(NC(=O)[C@H]2CC[C@H](C(=O)O)CC2)c1F] |
| I-104 | 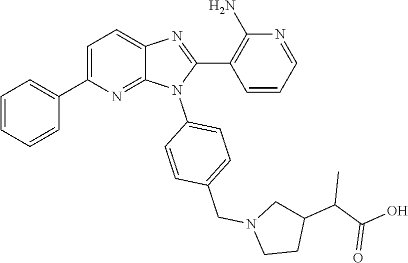 [SMILES: CC(C(=O)O)C1CCN(Cc2ccc(-n3c(-c4cccnc4N)nc4ccc(-c5ccccc5)nc43)cc2)C1] |
| I-105 | 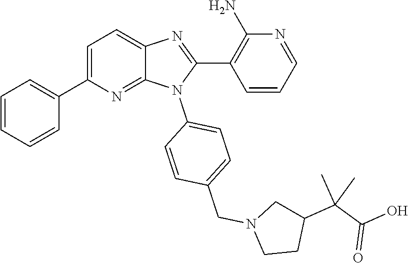 [SMILES: CC(C)(C(=O)O)C1CCN(Cc2ccc(-n3c(-c4cccnc4N)nc4ccc(-c5ccccc5)nc43)cc2)C1] |
| I-106 | 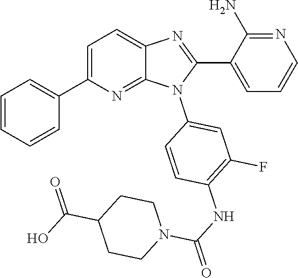 [SMILES: Nc1ncccc1-c1nc2ccc(-c3ccccc3)nc2n1-c1ccc(NC(=O)N2CCC(C(=O)O)CC2)c(F)c1] |
| I-107 | 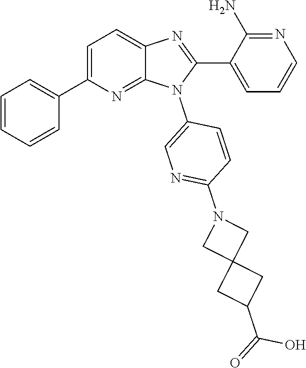 [SMILES: Nc1ncccc1-c1nc2ccc(-c3ccccc3)nc2n1-c1ccc(N2CC3(CC(C(=O)O)C3)C2)nc1] |
| I-108 | 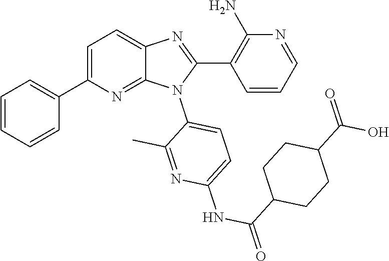 [SMILES: Cc1nc(NC(=O)C2CCC(C(=O)O)CC2)ccc1-n1c(-c2cccnc2N)nc2ccc(-c3ccccc3)nc21] |
| I-109 | 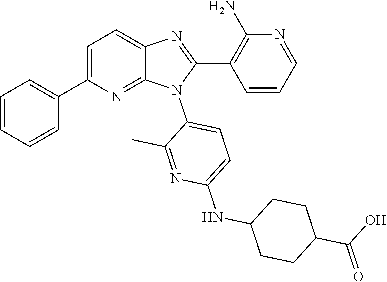 [SMILES: Cc1nc(NC2CCC(C(=O)O)CC2)ccc1-n1c(-c2cccnc2N)nc2ccc(-c3ccccc3)nc21] |
| I-110 | 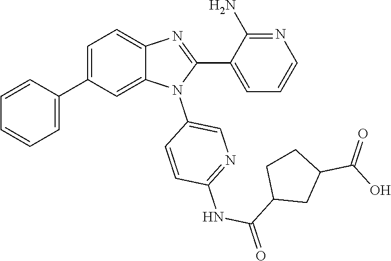 [SMILES: Nc1ncccc1-c1nc2ccc(-c3ccccc3)cc2n1-c1ccc(NC(=O)C2CCC(C(=O)O)C2)nc1] |
| I-111 | 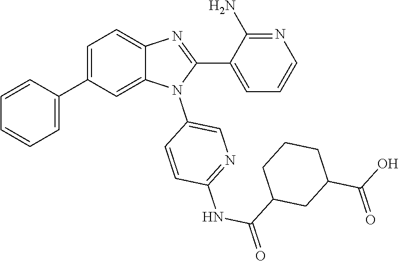 [SMILES: Nc1ncccc1-c1nc2ccc(-c3ccccc3)cc2n1-c1ccc(NC(=O)C2CCCC(C(=O)O)C2)nc1] |
| I-112 | 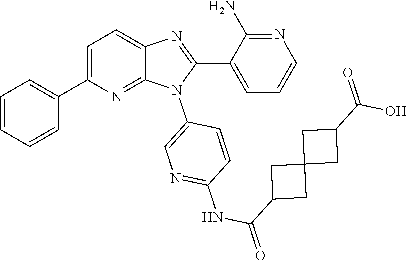 [SMILES: Nc1ncccc1-c1nc2ccc(-c3ccccc3)nc2n1-c1ccc(NC(=O)C2CC3(CC(C(=O)O)C3)C2)nc1] |
| I-113 | 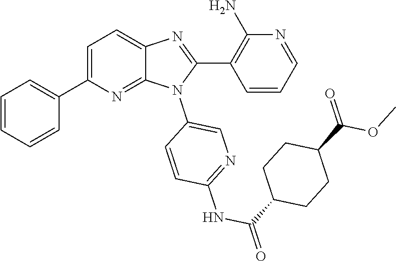 [SMILES: COC(=O)[C@H]1CC[C@H](C(=O)Nc2ccc(-n3c(-c4cccnc4N)nc4ccc(-c5ccccc5)nc43)cn2)CC1] |
| I-114 | 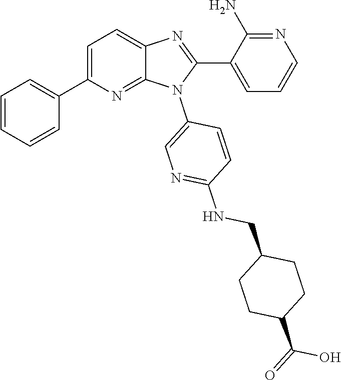 |
| I-115 | 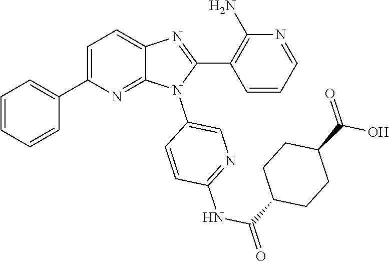 [SMILES: Nc1ncccc1-c1nc2ccc(-c3ccccc3)nc2n1-c1ccc(NC(=O)[C@H]2CC[C@H](C(=O)O)CC2)nc1] |
| I-116 | 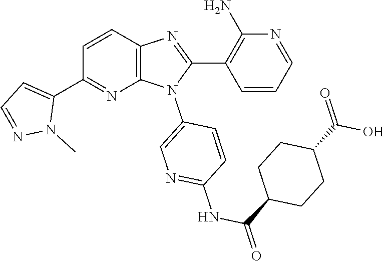 [SMILES: Cn1nccc1-c1ccc2nc(-c3cccnc3N)n(-c3ccc(NC(=O)[C@H]4CC[C@H](C(=O)O)CC4)nc3)c2n1] |
| I-117 | 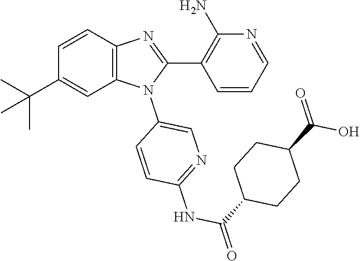 [SMILES: CC(C)(C)c1ccc2nc(-c3cccnc3N)n(-c3ccc(NC(=O)[C@H]4CC[C@H](C(=O)O)CC4)nc3)c2c1] |
| I-118 | 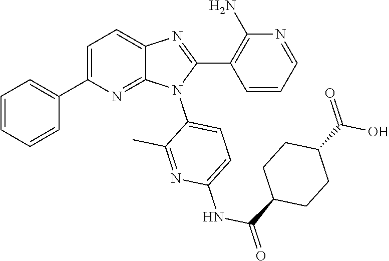 [SMILES: Cc1nc(NC(=O)[C@H]2CC[C@H](C(=O)O)CC2)ccc1-n1c(-c2cccnc2N)nc2ccc(-c3ccccc3)nc21] |
| I-119 | 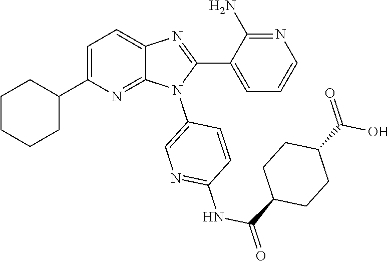 [SMILES: Nc1ncccc1-c1nc2ccc(C3CCCCC3)nc2n1-c1ccc(NC(=O)[C@H]2CC[C@H](C(=O)O)CC2)nc1] |
| I-120 | 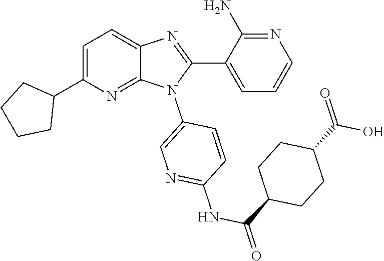 [SMILES: Nc1ncccc1-c1nc2ccc(C3CCCC3)nc2n1-c1ccc(NC(=O)[C@H]2CC[C@H](C(=O)O)CC2)nc1] |
| I-121 | 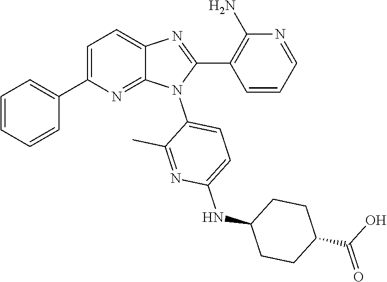 [SMILES: Cc1nc(N[C@H]2CC[C@H](C(=O)O)CC2)ccc1-n1c(-c2cccnc2N)nc2ccc(-c3ccccc3)nc21] |
| I-122 | 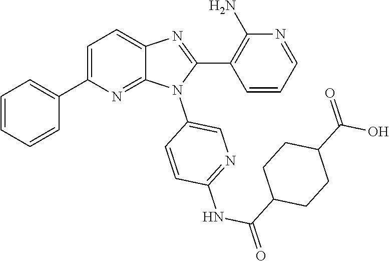 [SMILES: Nc1ncccc1-c1nc2ccc(-c3ccccc3)nc2n1-c1ccc(NC(=O)C2CCC(C(=O)O)CC2)nc1] |
| I-123 | 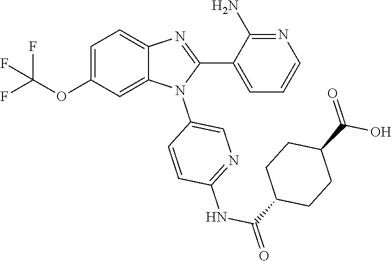 [SMILES: Nc1ncccc1-c1nc2ccc(OC(F)(F)F)cc2n1-c1ccc(NC(=O)[C@H]2CC[C@H](C(=O)O)CC2)nc1] |
| I-124 | 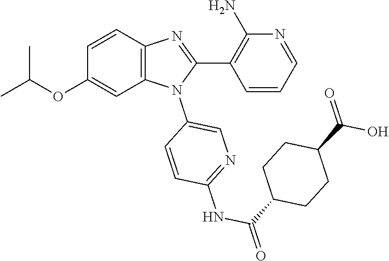 [SMILES: CC(C)Oc1ccc2nc(-c3cccnc3N)n(-c3ccc(NC(=O)[C@H]4CC[C@H](C(=O)O)CC4)nc3)c2c1] |
| I-125 | 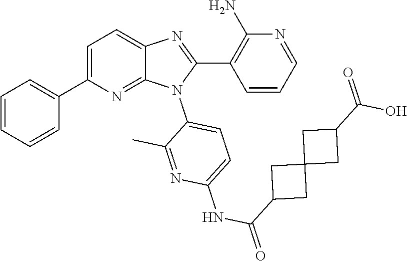 [SMILES: Cc1nc(NC(=O)C2CC3(CC(C(=O)O)C3)C2)ccc1-n1c(-c2cccnc2N)nc2ccc(-c3ccccc3)nc21] |
| I-126 | 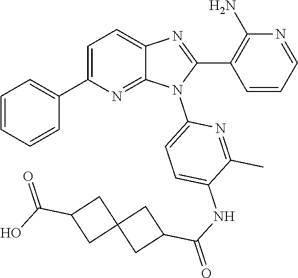 [SMILES: Cc1nc(-n2c(-c3cccnc3N)nc3ccc(-c4ccccc4)nc32)ccc1NC(=O)C1CC2(CC(C(=O)O)C2)C1] |
| I-127 | 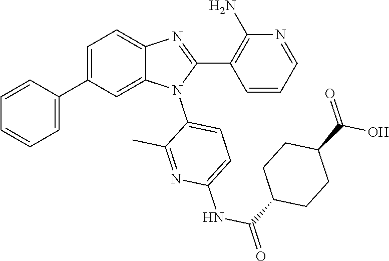 [SMILES: Cc1nc(NC(=O)[C@H]2CC[C@H](C(=O)O)CC2)ccc1-n1c(-c2cccnc2N)nc2ccc(-c3ccccc3)cc21] |
| I-128 | 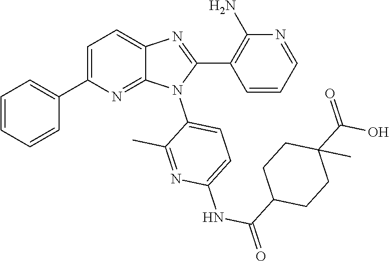 [SMILES: Cc1nc(NC(=O)C2CCC(C)(C(=O)O)CC2)ccc1-n1c(-c2cccnc2N)nc2ccc(-c3ccccc3)nc21] |
| I-129 | 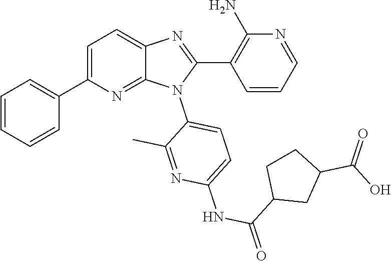 [SMILES: Cc1nc(NC(=O)C2CCC(C(=O)O)C2)ccc1-n1c(-c2cccnc2N)nc2ccc(-c3ccccc3)nc21] |
| I-130 | 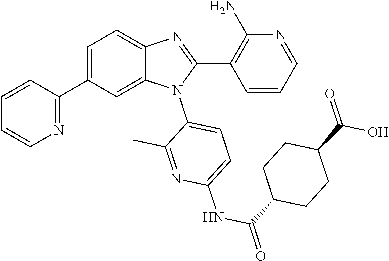 [SMILES: Cc1nc(NC(=O)[C@H]2CC[C@H](C(=O)O)CC2)ccc1-n1c(-c2cccnc2N)nc2ccc(-c3ccccn3)cc21] |
| I-131 | 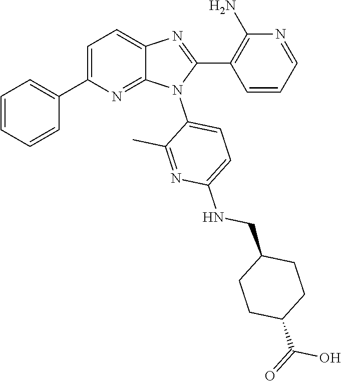 [SMILES: Cc1nc(NC[C@H]2CC[C@H](C(=O)O)CC2)ccc1-n1c(-c2cccnc2N)nc2ccc(-c3ccccc3)nc21] |
| I-132 | 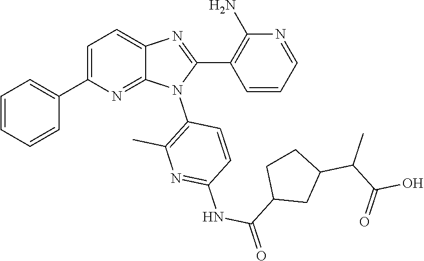 [SMILES: Cc1nc(NC(=O)C2CCC(C(C)C(=O)O)C2)ccc1-n1c(-c2cccnc2N)nc2ccc(-c3ccccc3)nc21] |
| I-133 | 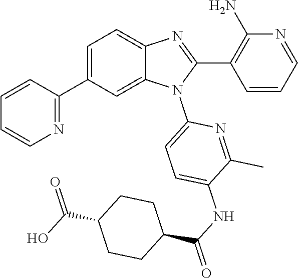 [SMILES: Cc1nc(-n2c(-c3cccnc3N)nc3ccc(-c4ccccn4)cc32)ccc1NC(=O)[C@H]1CC[C@H](C(=O)O)CC1] |
| I-134 | 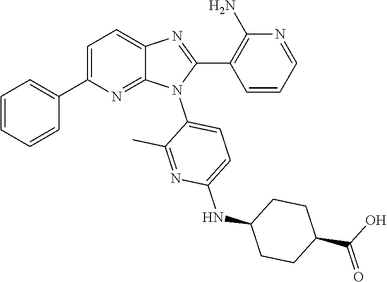 [SMILES: Cc1nc(N[C@H]2CC[C@@H](C(=O)O)CC2)ccc1-n1c(-c2cccnc2N)nc2ccc(-c3ccccc3)nc21] |
| I-135 | 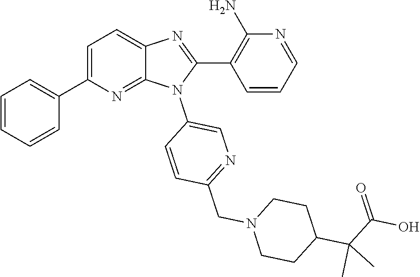 [SMILES: CC(C)(C(=O)O)C1CCN(Cc2ccc(-n3c(-c4cccnc4N)nc4ccc(-c5ccccc5)nc43)cn2)CC1] |
| I-136 | 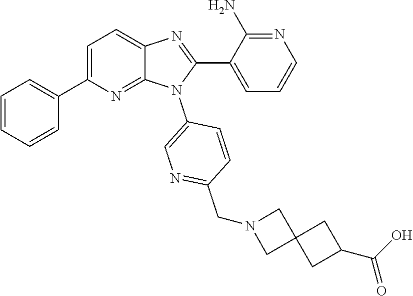 [SMILES: Nc1ncccc1-c1nc2ccc(-c3ccccc3)nc2n1-c1ccc(CN2CC3(CC(C(=O)O)C3)C2)nc1] |
| I-137 | 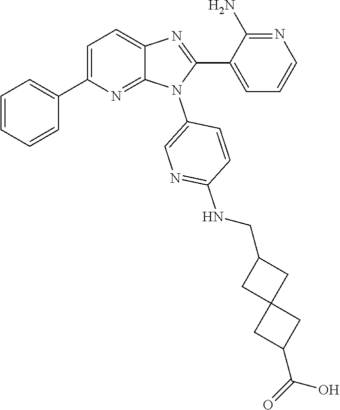 [SMILES: Nc1ncccc1-c1nc2ccc(-c3ccccc3)nc2n1-c1ccc(NCC2CC3(C2)CC(C(=O)O)C3)nc1] |
| I-138 | 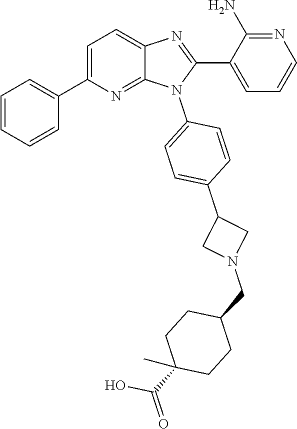 [SMILES: C[C@]1(C(=O)O)CC[C@H](CN2CC(c3ccc(-n4c(-c5cccnc5N)nc5ccc(-c6ccccc6)nc54)cc3)C2)CC1] |
| I-139 | 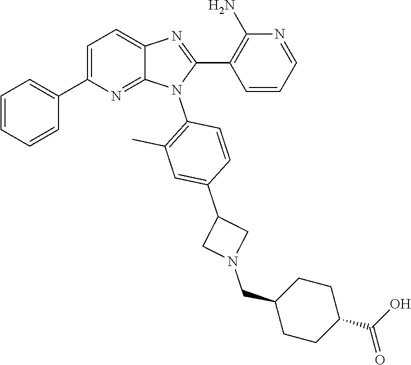 [SMILES: Cc1cc(C2CN(C[C@H]3CC[C@H](C(=O)O)CC3)C2)ccc1-n1c(-c2cccnc2N)nc2ccc(-c3ccccc3)nc21] |
| I-140 | 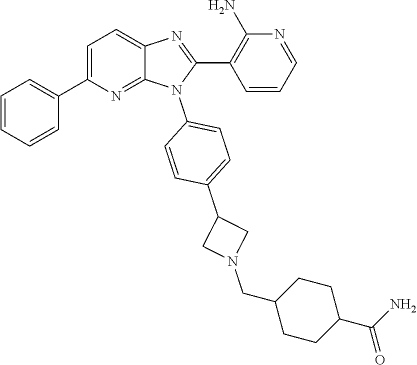 [SMILES: NC(=O)C1CCC(CN2CC(c3ccc(-n4c(-c5cccnc5N)nc5ccc(-c6ccccc6)nc54)cc3)C2)CC1] |
| I-141 | 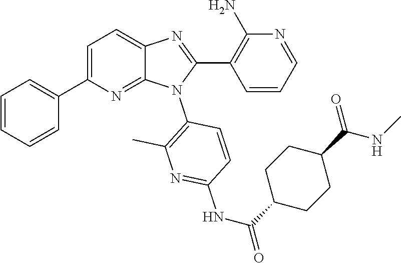 [SMILES: CNC(=O)[C@H]1CC[C@H](C(=O)Nc2ccc(-n3c(-c4cccnc4N)nc4ccc(-c5ccccc5)nc43)c(C)n2)CC1] |
| I-142 | 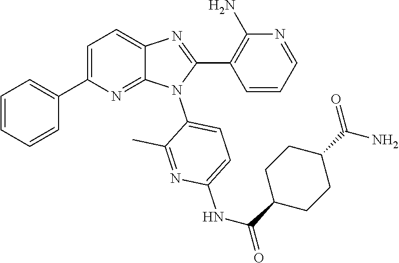 [SMILES: Cc1nc(NC(=O)[C@H]2CC[C@H](C(N)=O)CC2)ccc1-n1c(-c2cccnc2N)nc2ccc(-c3ccccc3)nc21] |
| I-143 | 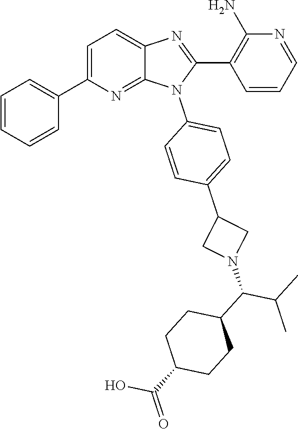 [SMILES: CC(C)[C@H]([C@H]1CC[C@H](C(=O)O)CC1)N1CC(c2ccc(-n3c(-c4cccnc4N)nc4ccc(-c5ccccc5)nc43)cc2)C1] |
| I-144 | 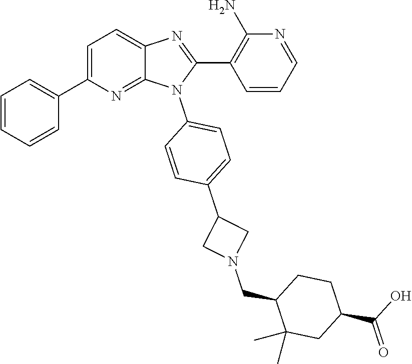 [SMILES: CC1(C)C[C@H](C(=O)O)CC[C@@H]1CN1CC(c2ccc(-n3c(-c4cccnc4N)nc4ccc(-c5ccccc5)nc43)cc2)C1] |
| I-145 | 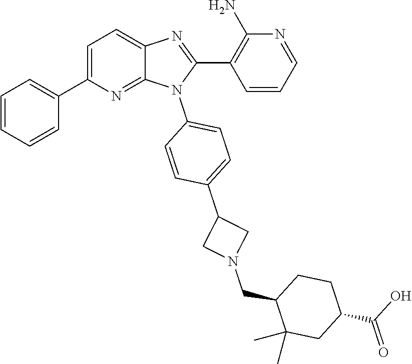 [SMILES: CC1(C)C[C@@H](C(=O)O)CC[C@@H]1CN1CC(c2ccc(-n3c(-c4cccnc4N)nc4ccc(-c5ccccc5)nc43)cc2)C1] |
| I-146 | 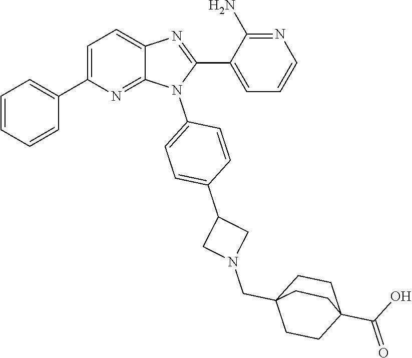 [SMILES: Nc1ncccc1-c1nc2ccc(-c3ccccc3)nc2n1-c1ccc(C2CN(CC34CCC(C(=O)O)(CC3)CC4)C2)cc1] |
| I-147 | 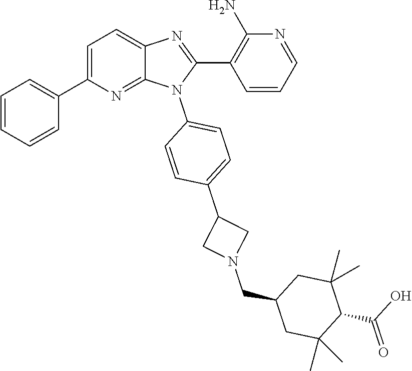 [SMILES: CC1(C)C[C@H](CN2CC(c3ccc(-n4c(-c5cccnc5N)nc5ccc(-c6ccccc6)nc54)cc3)C2)CC(C)(C)[C@H]1C(=O)O] |
| I-148 | 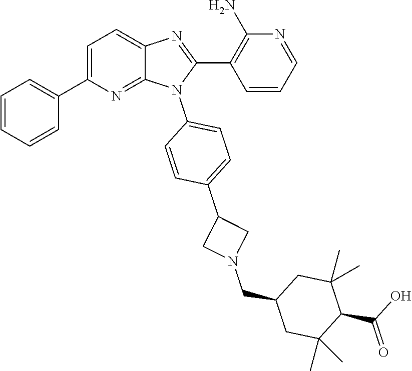 [SMILES: CC1(C)C[C@H](CN2CC(c3ccc(-n4c(-c5cccnc5N)nc5ccc(-c6ccccc6)nc54)cc3)C2)CC(C)(C)[C@@H]1C(=O)O] |
| I-149 | 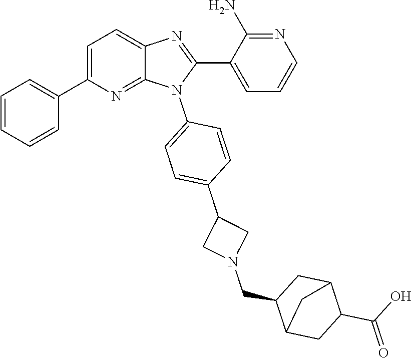 [SMILES: Nc1ncccc1-c1nc2ccc(-c3ccccc3)nc2n1-c1ccc(C2CN(C[C@H]3CC4CC3CC4C(=O)O)C2)cc1] |
| I-150 | 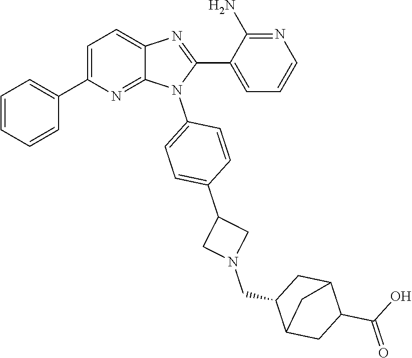 [SMILES: Nc1ncccc1-c1nc2ccc(-c3ccccc3)nc2n1-c1ccc(C2CN(C[C@@H]3CC4CC3CC4C(=O)O)C2)cc1] |
| I-151 | 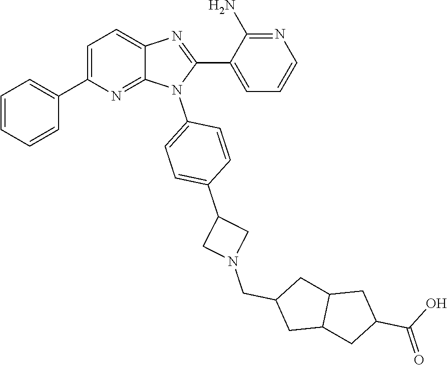 [SMILES: Nc1ncccc1-c1nc2ccc(-c3ccccc3)nc2n1-c1ccc(C2CN(CC3CC4CC(C(=O)O)CC4C3)C2)cc1] |
| I-152 | 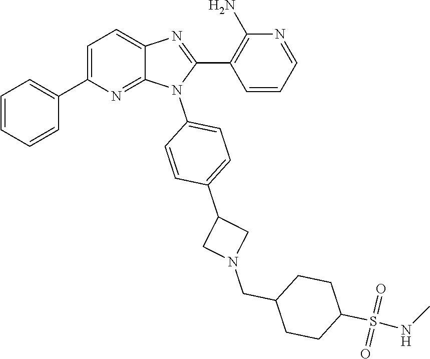 [SMILES: CNS(=O)(=O)C1CCC(CN2CC(c3ccc(-n4c(-c5cccnc5N)nc5ccc(-c6ccccc6)nc54)cc3)C2)CC1] |
| I-153 | 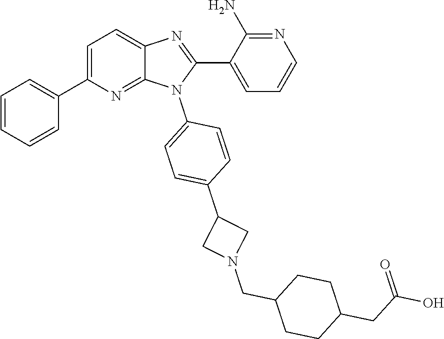 [SMILES: Nc1ncccc1-c1nc2ccc(-c3ccccc3)nc2n1-c1ccc(C2CN(CC3CCC(CC(=O)O)CC3)C2)cc1] |
| I-154 | 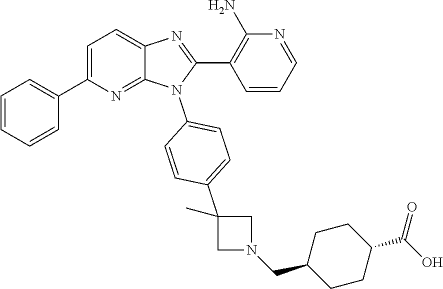 [SMILES: CC1(c2ccc(-n3c(-c4cccnc4N)nc4ccc(-c5ccccc5)nc43)cc2)CN(C[C@H]2CC[C@H](C(=O)O)CC2)C1] |
| I-155 | 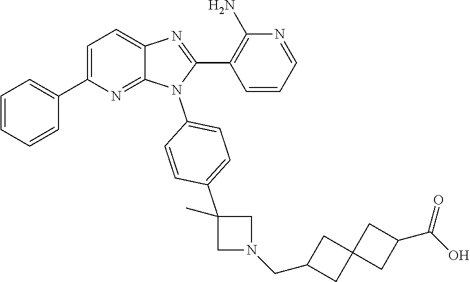 [SMILES: CC1(c2ccc(-n3c(-c4cccnc4N)nc4ccc(-c5ccccc5)nc43)cc2)CN(CC2CC3(C2)CC(C(=O)O)C3)C1] |
| I-156 | 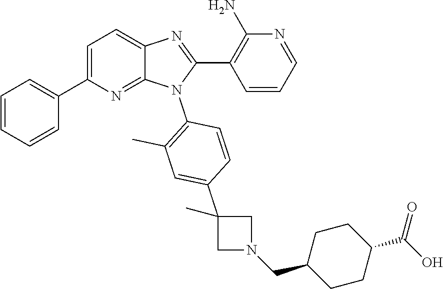 [SMILES: Cc1cc(C2(C)CN(C[C@H]3CC[C@H](C(=O)O)CC3)C2)ccc1-n1c(-c2cccnc2N)nc2ccc(-c3ccccc3)nc21] |
| I-157 | 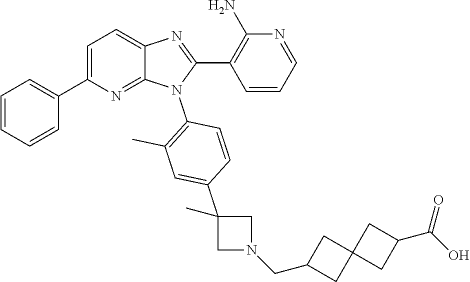 [SMILES: Cc1cc(C2(C)CN(CC3CC4(C3)CC(C(=O)O)C4)C2)ccc1-n1c(-c2cccnc2N)nc2ccc(-c3ccccc3)nc21] |
| I-158 | 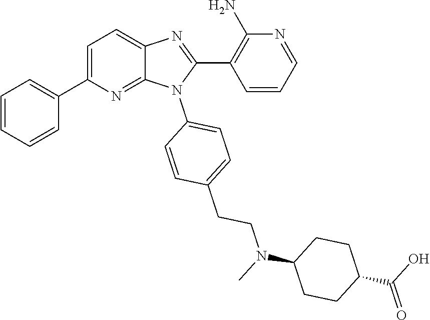 [SMILES: CN(CCc1ccc(-n2c(-c3cccnc3N)nc3ccc(-c4ccccc4)nc32)cc1)[C@H]1CC[C@H](C(=O)O)CC1] |
| I-159 | 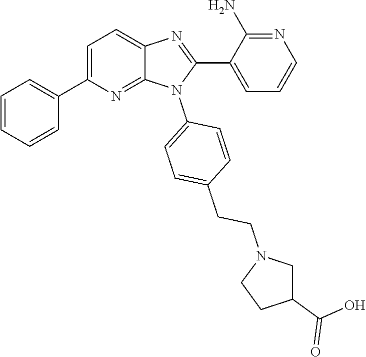 [SMILES: Nc1ncccc1-c1nc2ccc(-c3ccccc3)nc2n1-c1ccc(CCN2CCC(C(=O)O)C2)cc1] |
| I-160 | 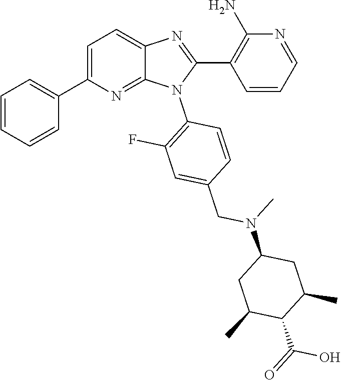 [SMILES: C[C@@H]1C[C@H](N(C)Cc2ccc(-n3c(-c4cccnc4N)nc4ccc(-c5ccccc5)nc43)c(F)c2)C[C@H](C)[C@H]1C(=O)O] |
| I-161 | 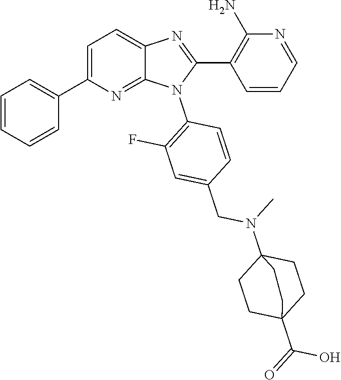 [SMILES: CN(Cc1ccc(-n2c(-c3cccnc3N)nc3ccc(-c4ccccc4)nc32)c(F)c1)C12CCC(C(=O)O)(CC1)CC2] |
| I-162 | 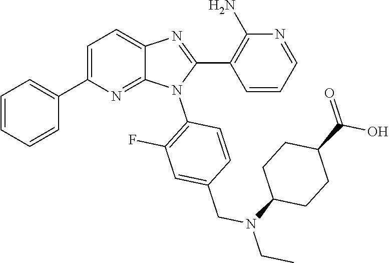 [SMILES: CCN(Cc1ccc(-n2c(-c3cccnc3N)nc3ccc(-c4ccccc4)nc32)c(F)c1)[C@H]1CC[C@@H](C(=O)O)CC1] |
| I-163 | 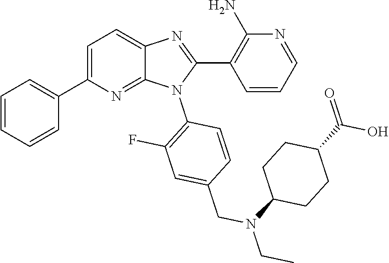 [SMILES: CCN(Cc1ccc(-n2c(-c3cccnc3N)nc3ccc(-c4ccccc4)nc32)c(F)c1)[C@H]1CC[C@H](C(=O)O)CC1] |
| I-164 | 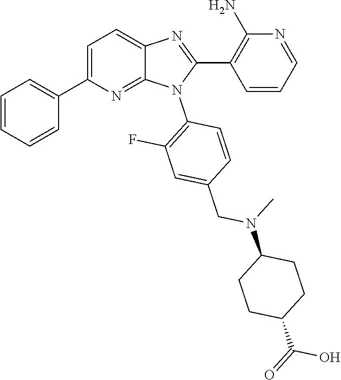 [SMILES: CN(Cc1ccc(-n2c(-c3cccnc3N)nc3ccc(-c4ccccc4)nc32)c(F)c1)[C@H]1CC[C@H](C(=O)O)CC1] |
| I-165 | 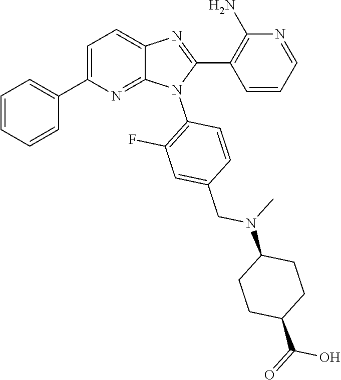 [SMILES: CN(Cc1ccc(-n2c(-c3cccnc3N)nc3ccc(-c4ccccc4)nc32)c(F)c1)[C@H]1CC[C@@H](C(=O)O)CC1] |
| I-166 | 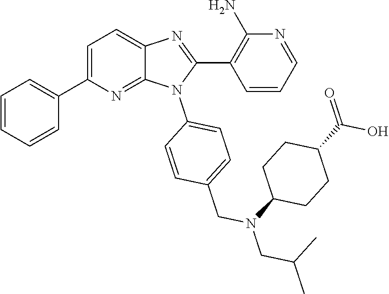 [SMILES: CC(C)CN(Cc1ccc(-n2c(-c3cccnc3N)nc3ccc(-c4ccccc4)nc32)cc1)[C@H]1CC[C@H](C(=O)O)CC1] |
| I-167 | 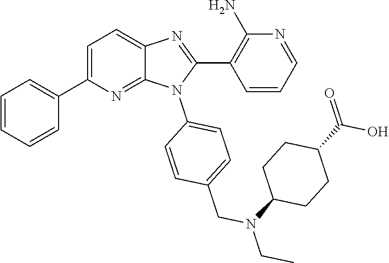 [SMILES: CCN(Cc1ccc(-n2c(-c3cccnc3N)nc3ccc(-c4ccccc4)nc32)cc1)[C@H]1CC[C@H](C(=O)O)CC1] |
| I-168 | 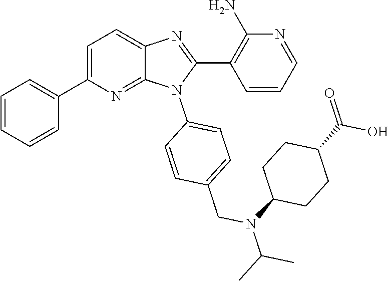 [SMILES: CC(C)N(Cc1ccc(-n2c(-c3cccnc3N)nc3ccc(-c4ccccc4)nc32)cc1)[C@H]1CC[C@H](C(=O)O)CC1] |
| I-169 | 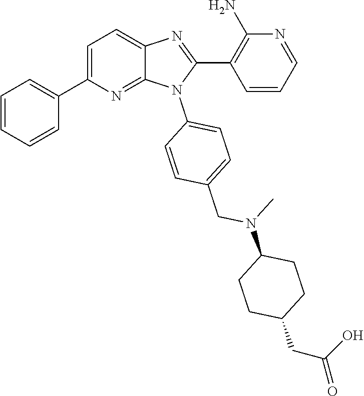 [SMILES: CN(Cc1ccc(-n2c(-c3cccnc3N)nc3ccc(-c4ccccc4)nc32)cc1)[C@H]1CC[C@H](CC(=O)O)CC1] |
| I-170 | 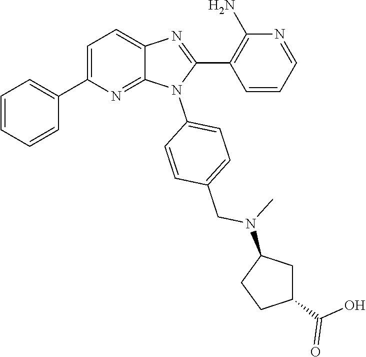 [SMILES: CN(Cc1ccc(-n2c(-c3cccnc3N)nc3ccc(-c4ccccc4)nc32)cc1)[C@@H]1CC[C@@H](C(=O)O)C1] |
| T-171 | 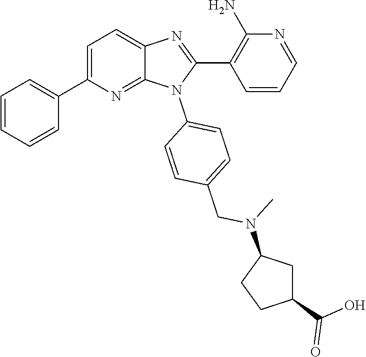 [SMILES: CN(Cc1ccc(-n2c(-c3cccnc3N)nc3ccc(-c4ccccc4)nc32)cc1)[C@@H]1CC[C@H](C(=O)O)C1] |
| I-172 | 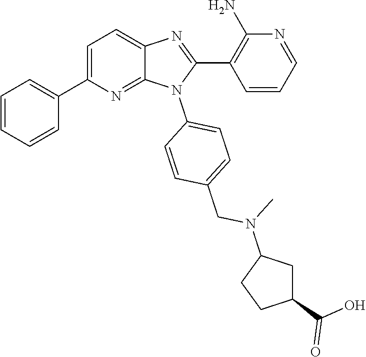 [SMILES: CN(Cc1ccc(-n2c(-c3cccnc3N)nc3ccc(-c4ccccc4)nc32)cc1)C1CC[C@H](C(=O)O)C1] |
| I-173 | 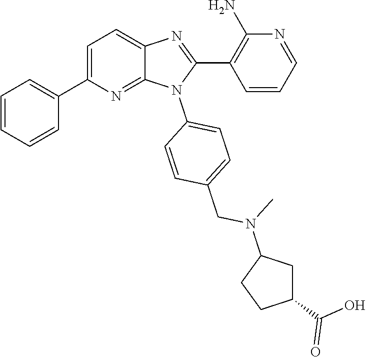 [SMILES: CN(Cc1ccc(-n2c(-c3cccnc3N)nc3ccc(-c4ccccc4)nc32)cc1)C1CC[C@@H](C(=O)O)C1] |
| I-174 | 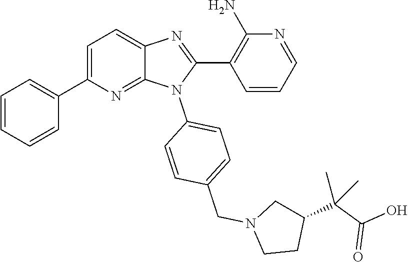 [SMILES: CC(C)(C(=O)O)[C@@H]1CCN(Cc2ccc(-n3c(-c4cccnc4N)nc4ccc(-c5ccccc5)nc43)cc2)C1] |
| I-175 | 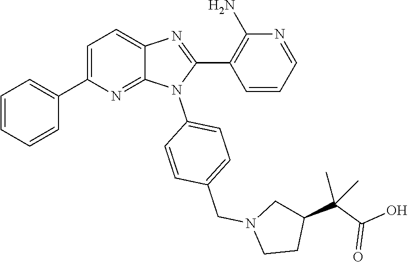 [SMILES: CC(C)(C(=O)O)[C@H]1CCN(Cc2ccc(-n3c(-c4cccnc4N)nc4ccc(-c5ccccc5)nc43)cc2)C1] |
| I-176 | 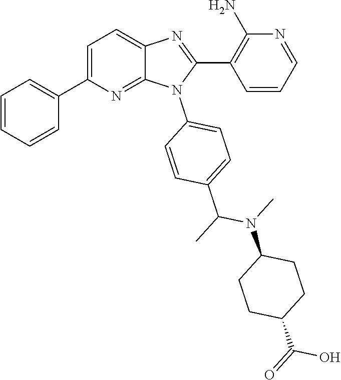 [SMILES: CC(c1ccc(-n2c(-c3cccnc3N)nc3ccc(-c4ccccc4)nc32)cc1)N(C)[C@H]1CC[C@H](C(=O)O)CC1] |
| I-177 | 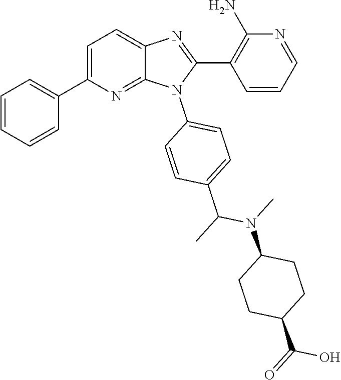 [SMILES: CC(c1ccc(-n2c(-c3cccnc3N)nc3ccc(-c4ccccc4)nc32)cc1)N(C)[C@H]1CC[C@@H](C(=O)O)CC1] |
| I-178 | 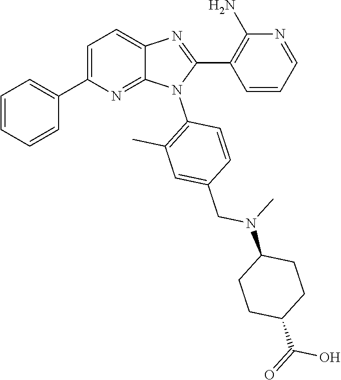 [SMILES: Cc1cc(CN(C)[C@H]2CC[C@H](C(=O)O)CC2)ccc1-n1c(-c2cccnc2N)nc2ccc(-c3ccccc3)nc21] |
| I-179 | 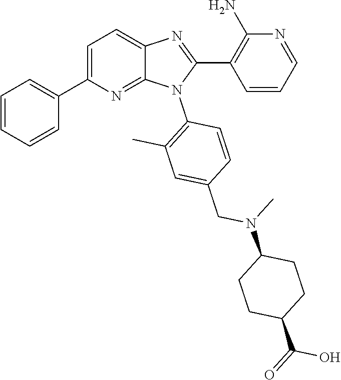 [SMILES: Cc1cc(CN(C)[C@H]2CC[C@@H](C(=O)O)CC2)ccc1-n1c(-c2cccnc2N)nc2ccc(-c3ccccc3)nc21] |
| I-180 | 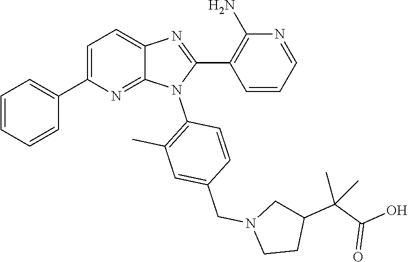 [SMILES: Cc1cc(CN2CCC(C(C)(C)C(=O)O)C2)ccc1-n1c(-c2cccnc2N)nc2ccc(-c3ccccc3)nc21] |
| I-181 | 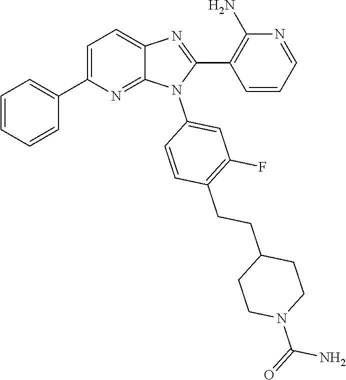 [SMILES: NC(=O)N1CCC(CCc2ccc(-n3c(-c4cccnc4N)nc4ccc(-c5ccccc5)nc43)cc2F)CC1] |
| I-182 | 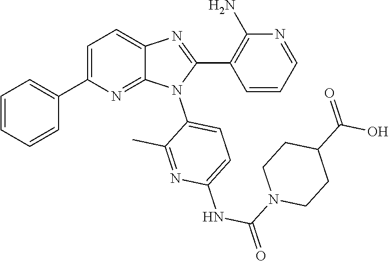 [SMILES: Cc1nc(NC(=O)N2CCC(C(=O)O)CC2)ccc1-n1c(-c2cccnc2N)nc2ccc(-c3ccccc3)nc21] |
| I-183 | 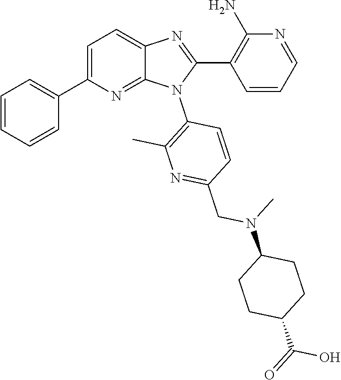 [SMILES: Cc1nc(CN(C)[C@H]2CC[C@H](C(=O)O)CC2)ccc1-n1c(-c2cccnc2N)nc2ccc(-c3ccccc3)nc21] |
| I-184 | 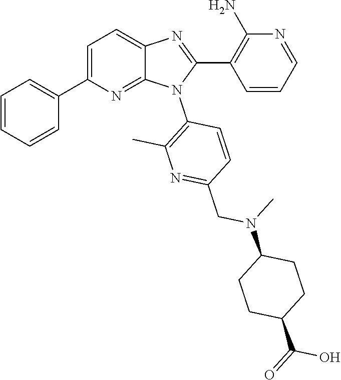 [SMILES: Cc1nc(CN(C)[C@H]2CC[C@@H](C(=O)O)CC2)ccc1-n1c(-c2cccnc2N)nc2ccc(-c3ccccc3)nc21] |
| I-185 | 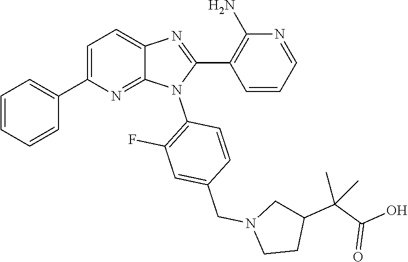 [SMILES: CC(C)(C(=O)O)C1CCN(Cc2ccc(-n3c(-c4cccnc4N)nc4ccc(-c5ccccc5)nc43)c(F)c2)C1] |
| I-186 | 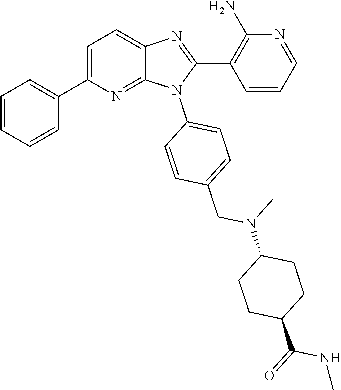 [SMILES: CNC(=O)[C@H]1CC[C@H](N(C)Cc2ccc(-n3c(-c4cccnc4N)nc4ccc(-c5ccccc5)nc43)cc2)CC1] |
| I-187 | 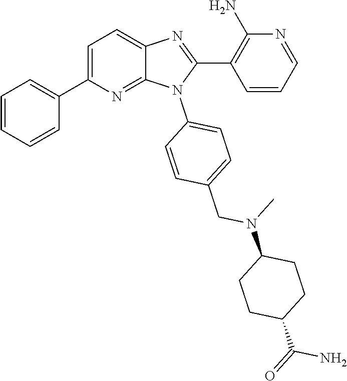 [SMILES: CN(Cc1ccc(-n2c(-c3cccnc3N)nc3ccc(-c4ccccc4)nc32)cc1)[C@H]1CC[C@H](C(N)=O)CC1] |
| I-188 | 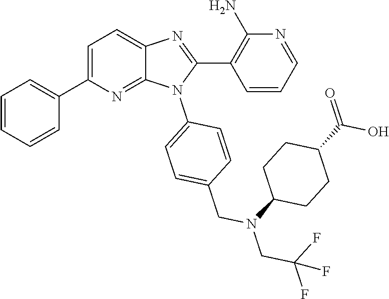 [SMILES: Nc1ncccc1-c1nc2ccc(-c3ccccc3)nc2n1-c1ccc(CN(CC(F)(F)F)[C@H]2CC[C@H](C(=O)O)CC2)cc1] |
| I-189 | 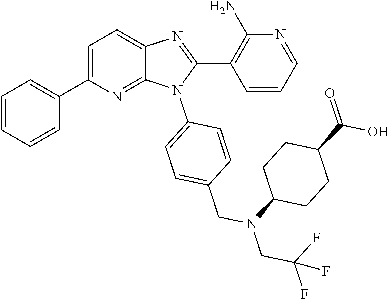 [SMILES: Nc1ncccc1-c1nc2ccc(-c3ccccc3)nc2n1-c1ccc(CN(CC(F)(F)F)[C@H]2CC[C@@H](C(=O)O)CC2)cc1] |
| I-190 | 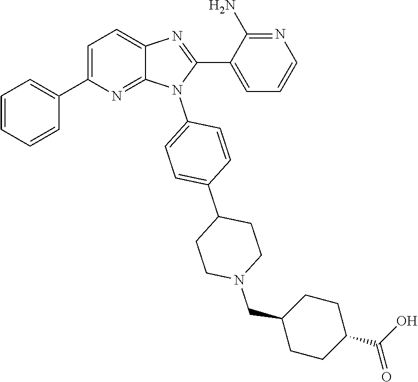 [SMILES: Nc1ncccc1-c1nc2ccc(-c3ccccc3)nc2n1-c1ccc(C2CCN(C[C@H]3CC[C@H](C(=O)O)CC3)CC2)cc1] |
| I-191 | 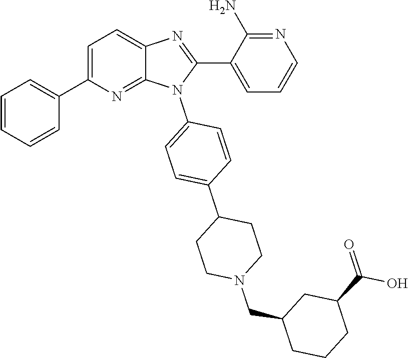 [SMILES: Nc1ncccc1-c1nc2ccc(-c3ccccc3)nc2n1-c1ccc(C2CCN(C[C@@H]3CCC[C@H](C(=O)O)C3)CC2)cc1] |
| I-192 | 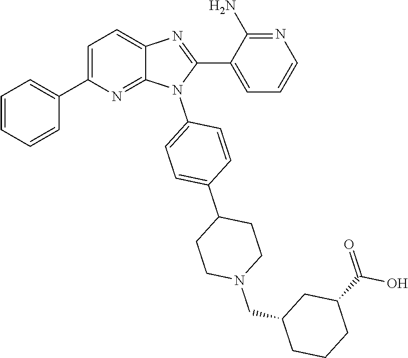 [SMILES: Nc1ncccc1-c1nc2ccc(-c3ccccc3)nc2n1-c1ccc(C2CCN(C[C@H]3CCC[C@@H](C(=O)O)C3)CC2)cc1] |
| I-193 | 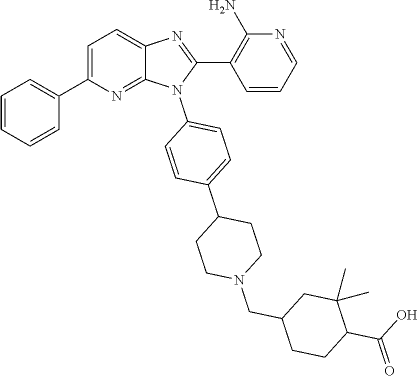 [SMILES: CC1(C)CC(CN2CCC(c3ccc(-n4c(-c5cccnc5N)nc5ccc(-c6ccccc6)nc54)cc3)CC2)CCC1C(=O)O] |
| I-194 | 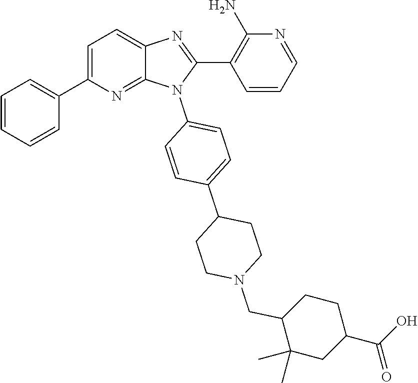 [SMILES: CC1(C)CC(C(=O)O)CCC1CN1CCC(c2ccc(-n3c(-c4cccnc4N)nc4ccc(-c5ccccc5)nc43)cc2)CC1] |
| I-195 | 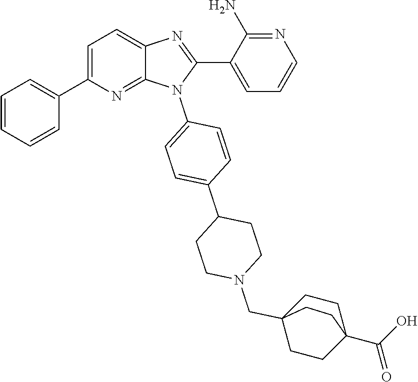 [SMILES: Nc1ncccc1-c1nc2ccc(-c3ccccc3)nc2n1-c1ccc(C2CCN(CC34CCC(C(=O)O)(CC3)CC4)CC2)cc1] |
| I-196 | 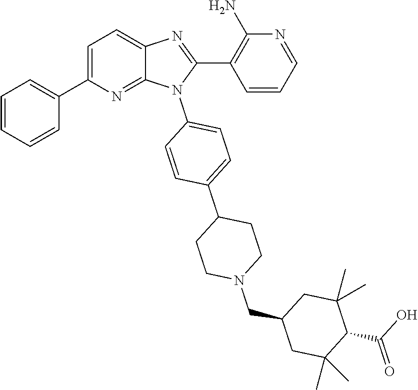 [SMILES: CC1(C)C[C@H](CN2CCC(c3ccc(-n4c(-c5cccnc5N)nc5ccc(-c6ccccc6)nc54)cc3)CC2)CC(C)(C)[C@H]1C(=O)O] |
| I-197 | 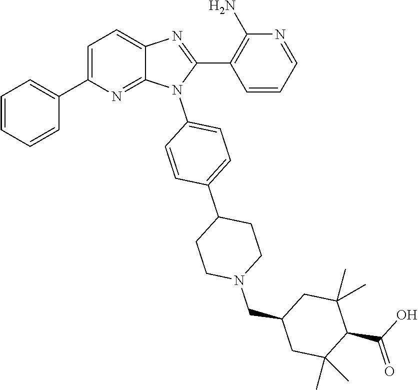 [SMILES: CC1(C)C[C@H](CN2CCC(c3ccc(-n4c(-c5cccnc5N)nc5ccc(-c6ccccc6)nc54)cc3)CC2)CC(C)(C)[C@@H]1C(=O)O] |
| I-198 | 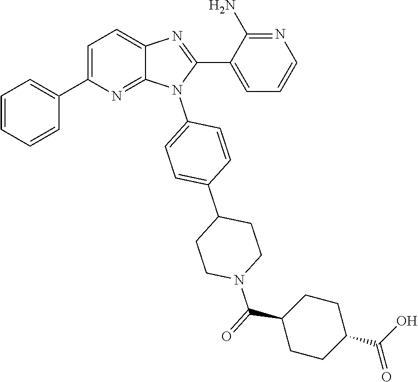 [SMILES: Nc1ncccc1-c1nc2ccc(-c3ccccc3)nc2n1-c1ccc(C2CCN(C(=O)[C@H]3CC[C@H](C(=O)O)CC3)CC2)cc1] |
| I-199 | 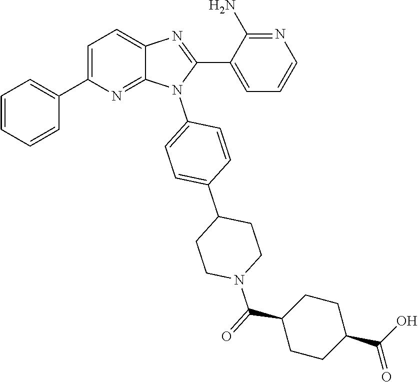 [SMILES: Nc1ncccc1-c1nc2ccc(-c3ccccc3)nc2n1-c1ccc(C2CCN(C(=O)[C@H]3CC[C@@H](C(=O)O)CC3)CC2)cc1] |
| I-200 | 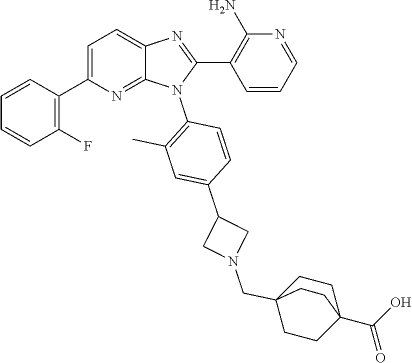 [SMILES: Cc1cc(C2CN(CC34CCC(C(=O)O)(CC3)CC4)C2)ccc1-n1c(-c2cccnc2N)nc2ccc(-c3ccccc3F)nc21] |
| I-201 | 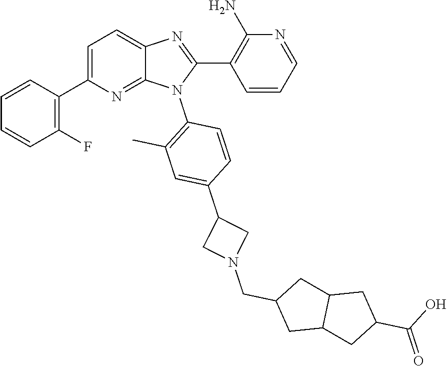 [SMILES: Cc1cc(C2CN(CC3CC4CC(C(=O)O)CC4C3)C2)ccc1-n1c(-c2cccnc2N)nc2ccc(-c3ccccc3F)nc21] |
| I-202 | 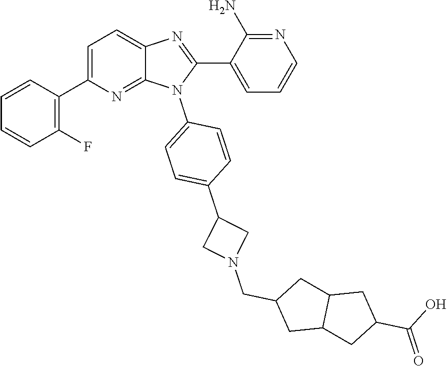 [SMILES: Nc1ncccc1-c1nc2ccc(-c3ccccc3F)nc2n1-c1ccc(C2CN(CC3CC4CC(C(=O)O)CC4C3)C2)cc1] |
| I-203 | 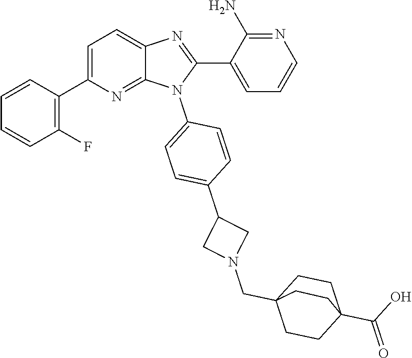 [SMILES: Nc1ncccc1-c1nc2ccc(-c3ccccc3F)nc2n1-c1ccc(C2CN(CC34CCC(C(=O)O)(CC3)CC4)C2)cc1] |
| I-204 | 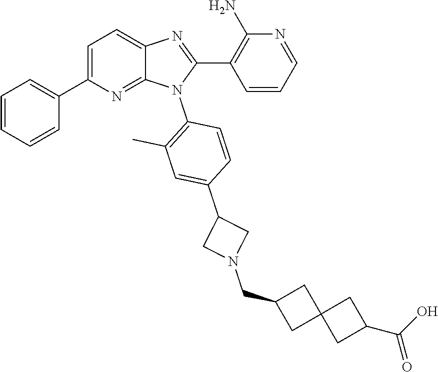 |
| I-205 | 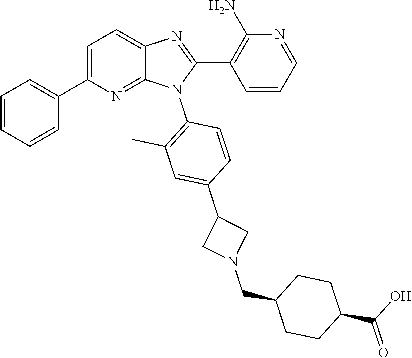 [SMILES: Cc1cc(C2CN(C[C@H]3CC[C@@H](C(=O)O)CC3)C2)ccc1-n1c(-c2cccnc2N)nc2ccc(-c3ccccc3)nc21] |
| I-206 | 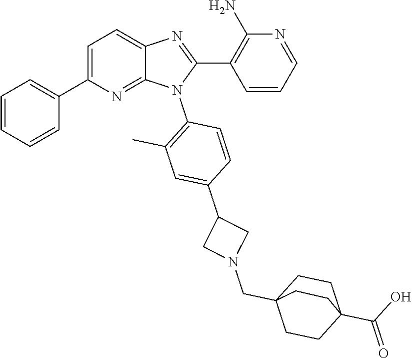 [SMILES: Cc1cc(C2CN(CC34CCC(C(=O)O)(CC3)CC4)C2)ccc1-n1c(-c2cccnc2N)nc2ccc(-c3ccccc3)nc21] |
| I-207 | 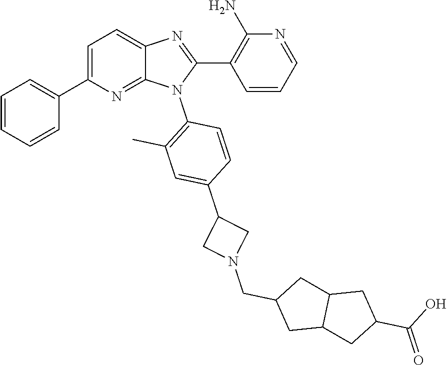 [SMILES: Cc1cc(C2CN(CC3CC4CC(C(=O)O)CC4C3)C2)ccc1-n1c(-c2cccnc2N)nc2ccc(-c3ccccc3)nc21] |
| I-208 | 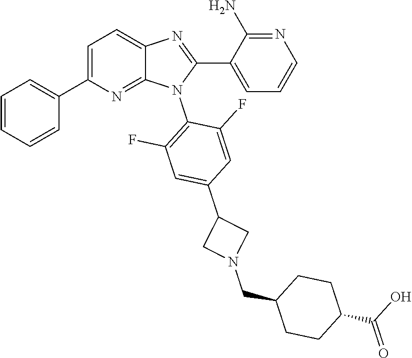 [SMILES: Nc1ncccc1-c1nc2ccc(-c3ccccc3)nc2n1-c1c(F)cc(C2CN(C[C@H]3CC[C@H](C(=O)O)CC3)C2)cc1F] |
| I-209 | 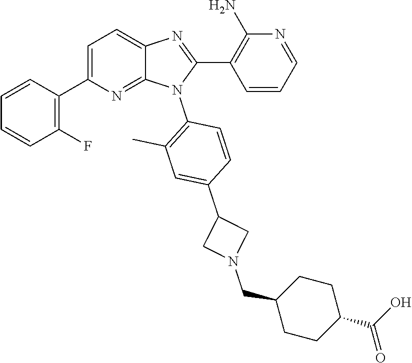 [SMILES: Cc1cc(C2CN(C[C@H]3CC[C@H](C(=O)O)CC3)C2)ccc1-n1c(-c2cccnc2N)nc2ccc(-c3ccccc3F)nc21] |
| I-210 | 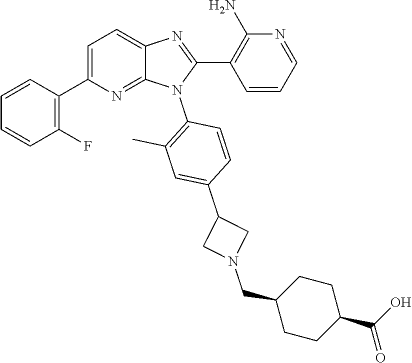 [SMILES: Cc1cc(C2CN(C[C@H]3CC[C@@H](C(=O)O)CC3)C2)ccc1-n1c(-c2cccnc2N)nc2ccc(-c3ccccc3F)nc21] |
| I-211 | 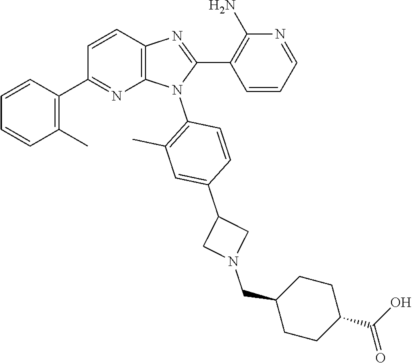 [SMILES: Cc1ccccc1-c1ccc2nc(-c3cccnc3N)n(-c3ccc(C4CN(C[C@H]5CC[C@H](C(=O)O)CC5)C4)cc3C)c2n1] |
| I-212 | 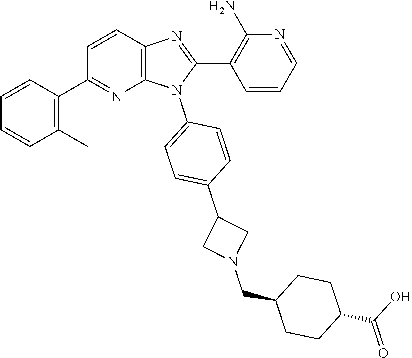 [SMILES: Cc1ccccc1-c1ccc2nc(-c3cccnc3N)n(-c3ccc(C4CN(C[C@H]5CC[C@H](C(=O)O)CC5)C4)cc3)c2n1] |
| I-213 | 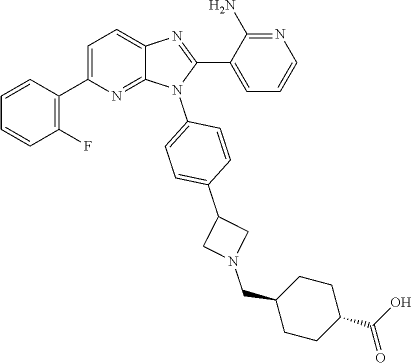 [SMILES: Nc1ncccc1-c1nc2ccc(-c3ccccc3F)nc2n1-c1ccc(C2CN(C[C@H]3CC[C@H](C(=O)O)CC3)C2)cc1] |
| I-214 | 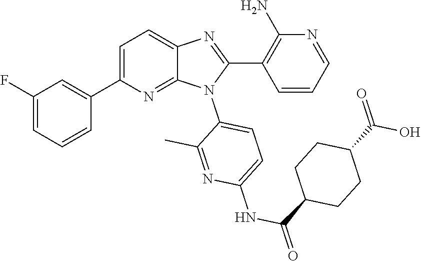 [SMILES: Cc1nc(NC(=O)[C@H]2CC[C@H](C(=O)O)CC2)ccc1-n1c(-c2cccnc2N)nc2ccc(-c3cccc(F)c3)nc21] |
| I-215 | 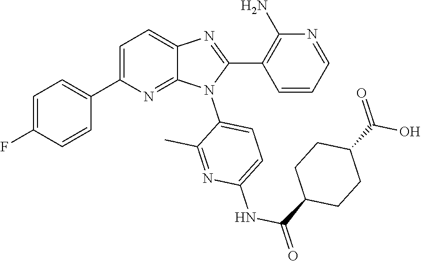 [SMILES: Cc1nc(NC(=O)[C@H]2CC[C@H](C(=O)O)CC2)ccc1-n1c(-c2cccnc2N)nc2ccc(-c3ccc(F)cc3)nc21] |
| I-216 | 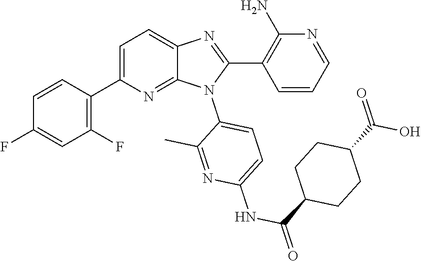 [SMILES: Cc1nc(NC(=O)[C@H]2CC[C@H](C(=O)O)CC2)ccc1-n1c(-c2cccnc2N)nc2ccc(-c3ccc(F)cc3F)nc21] |
| I-217 | 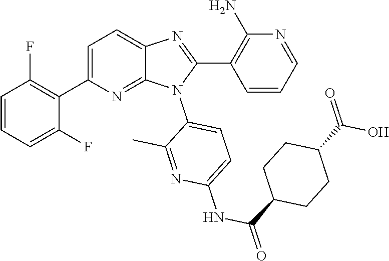 [SMILES: Cc1nc(NC(=O)[C@H]2CC[C@H](C(=O)O)CC2)ccc1-n1c(-c2cccnc2N)nc2ccc(-c3c(F)cccc3F)nc21] |
| I-218 | 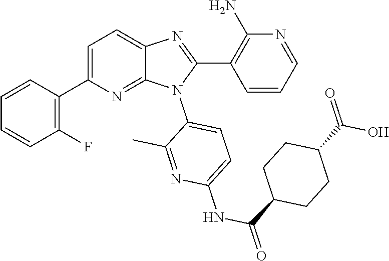 [SMILES: Cc1nc(NC(=O)[C@H]2CC[C@H](C(=O)O)CC2)ccc1-n1c(-c2cccnc2N)nc2ccc(-c3ccccc3F)nc21] |
| I-219 | 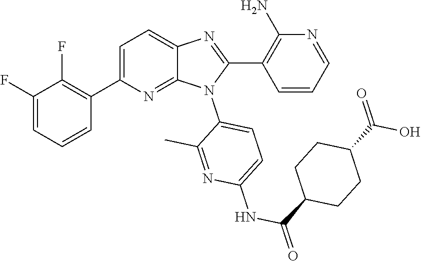 [SMILES: Cc1nc(NC(=O)[C@H]2CC[C@H](C(=O)O)CC2)ccc1-n1c(-c2cccnc2N)nc2ccc(-c3cccc(F)c3F)nc21] |
| I-220 | 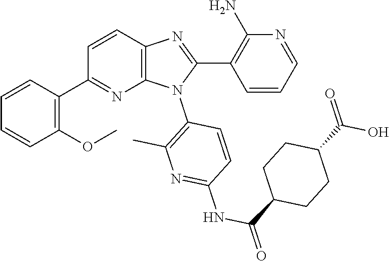 [SMILES: COc1ccccc1-c1ccc2nc(-c3cccnc3N)n(-c3ccc(NC(=O)[C@H]4CC[C@H](C(=O)O)CC4)nc3C)c2n1] |
| I-221 | 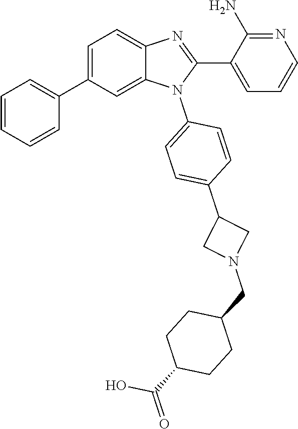 [SMILES: Nc1ncccc1-c1nc2ccc(-c3ccccc3)cc2n1-c1ccc(C2CN(C[C@H]3CC[C@H](C(=O)O)CC3)C2)cc1] |
| I-222 | 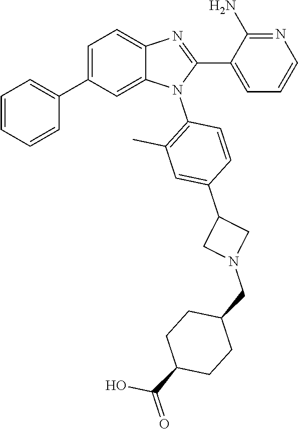 [SMILES: Cc1cc(C2CN(C[C@H]3CC[C@@H](C(=O)O)CC3)C2)ccc1-n1c(-c2cccnc2N)nc2ccc(-c3ccccc3)cc21] |
| I-223 | 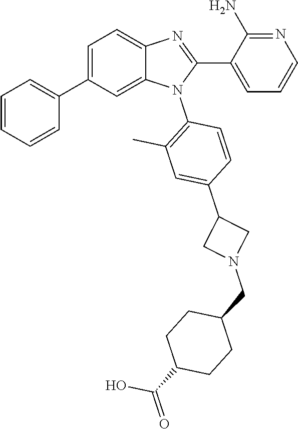 |
| I-224 | 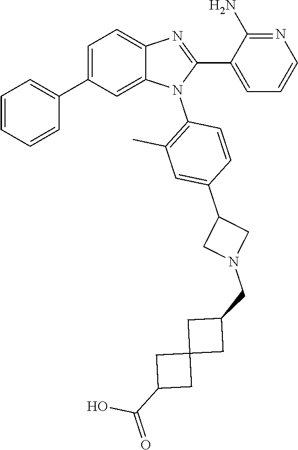 |
| I-225 | 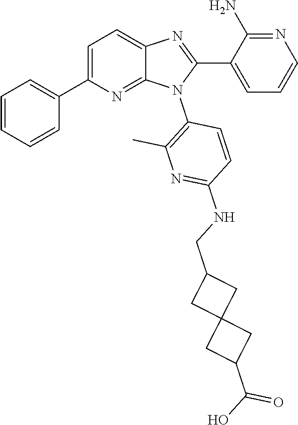 [SMILES: Cc1nc(NCC2CC3(C2)CC(C(=O)O)C3)ccc1-n1c(-c2cccnc2N)nc2ccc(-c3ccccc3)nc21] |
| I-226 | 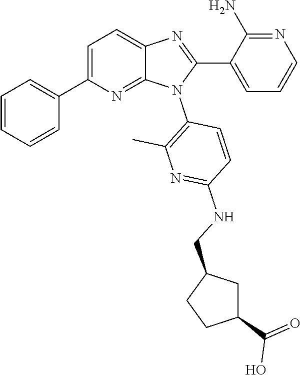 [SMILES: Cc1nc(NC[C@@H]2CC[C@H](C(=O)O)C2)ccc1-n1c(-c2cccnc2N)nc2ccc(-c3ccccc3)nc21] |
| I-227 | 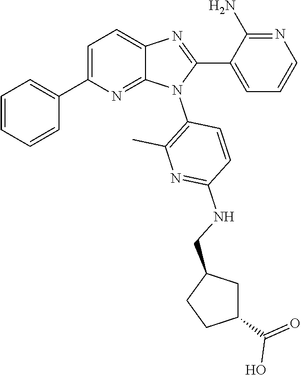 [SMILES: Cc1nc(NC[C@@H]2CC[C@@H](C(=O)O)C2)ccc1-n1c(-c2cccnc2N)nc2ccc(-c3ccccc3)nc21] |
| I-228 | 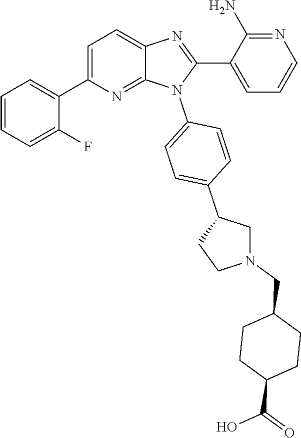 [SMILES: Nc1ncccc1-c1nc2ccc(-c3ccccc3F)nc2n1-c1ccc([C@H]2CCN(C[C@H]3CC[C@@H](C(=O)O)CC3)C2)cc1] |
| I-229 | 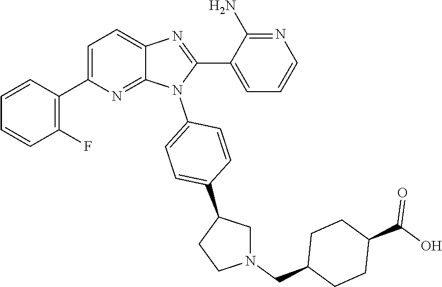 [SMILES: Nc1ncccc1-c1nc2ccc(-c3ccccc3F)nc2n1-c1ccc([C@@H]2CCN(C[C@H]3CC[C@@H](C(=O)O)CC3)C2)cc1] |
| I-230 | 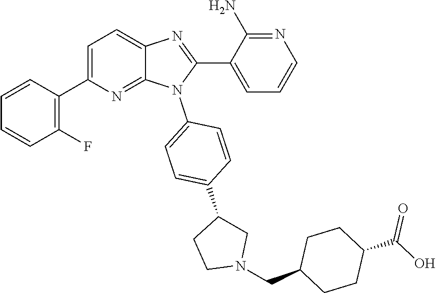 [SMILES: Nc1ncccc1-c1nc2ccc(-c3ccccc3F)nc2n1-c1ccc([C@H]2CCN(C[C@H]3CC[C@H](C(=O)O)CC3)C2)cc1] |
| I-231 | 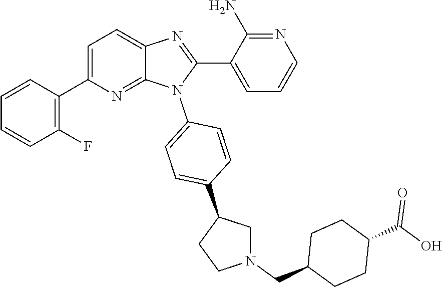 [SMILES: Nc1ncccc1-c1nc2ccc(-c3ccccc3F)nc2n1-c1ccc([C@@H]2CCN(C[C@H]3CC[C@H](C(=O)O)CC3)C2)cc1] |
| I-232 | 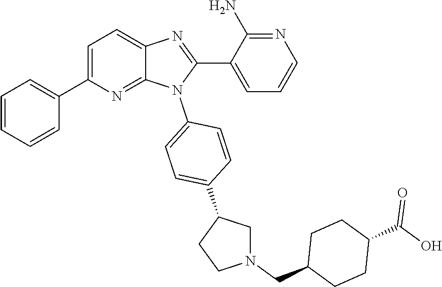 [SMILES: Nc1ncccc1-c1nc2ccc(-c3ccccc3)nc2n1-c1ccc([C@H]2CCN(C[C@H]3CC[C@H](C(=O)O)CC3)C2)cc1] |
| I-233 | 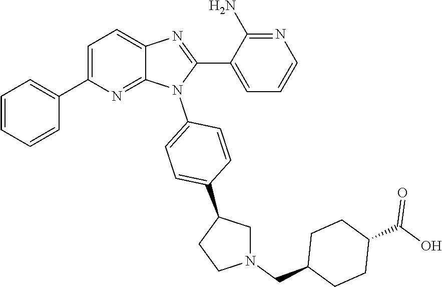 [SMILES: Nc1ncccc1-c1nc2ccc(-c3ccccc3)nc2n1-c1ccc([C@@H]2CCN(C[C@H]3CC[C@H](C(=O)O)CC3)C2)cc1] |
| I-234 | 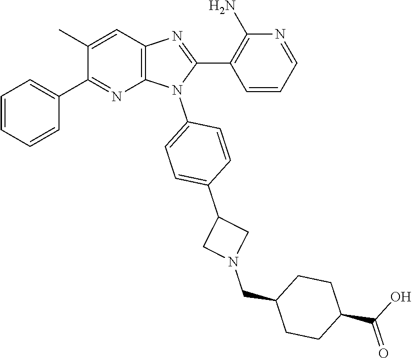 [SMILES: Cc1cc2nc(-c3cccnc3N)n(-c3ccc(C4CN(C[C@H]5CC[C@@H](C(=O)O)CC5)C4)cc3)c2nc1-c1ccccc1] |
| I-235 | 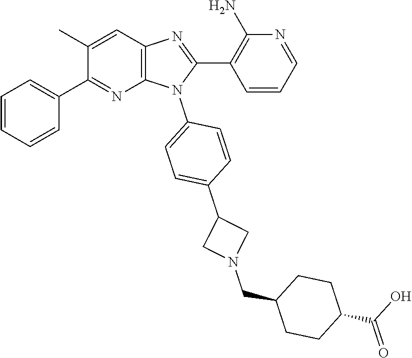 [SMILES: Cc1cc2nc(-c3cccnc3N)n(-c3ccc(C4CN(C[C@H]5CC[C@H](C(=O)O)CC5)C4)cc3)c2nc1-c1ccccc1] |

### Synthetic Methods

Methods for preparing compounds described herein are illustrated in the following synthetic schemes (Scheme 1-Scheme 6). The schemes are given for the purpose of illustrating the invention, and not intended to limit the scope or spirit of the invention. Starting materials shown in the schemes can be obtained from commercial sources or can be prepared based on procedures described in the literature.

Scheme 1 illustrates a general method for making aminopyridinyl imidazo[4,5-b]pyridine compounds G. Reaction of nitropyridine A and aldehyde B provides imidazo[4,5-b]pyridine C. Treatment of imidazo[4,5-b]pyridine C with acid removes the Boc protecting group to provide amine D. Reaction of amine D with carboxylic acid E under amide coupling conditions provides amide F. Treatment of amide F with lithium hydroxide in water provides making aminopyridinyl imidazo[4,5-b]pyridine compound G.

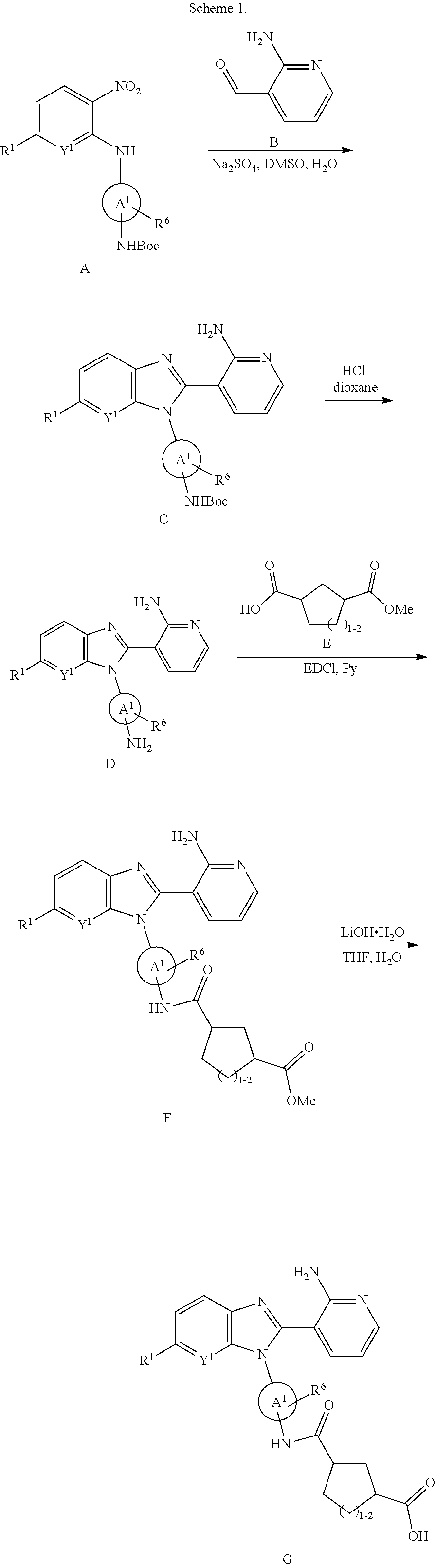

Scheme 2 illustrates a general method for preparing aminopyridinyl imidazo[4,5-b]pyridine compounds. Reaction of A with B provides C. Reaction of C with aldehyde D provides imidazo[4,5-b]pyridine E, which is then converted to the final aminopyridinyl imidazo[4,5-b]pyridine compound F, after optional hydrolysis.

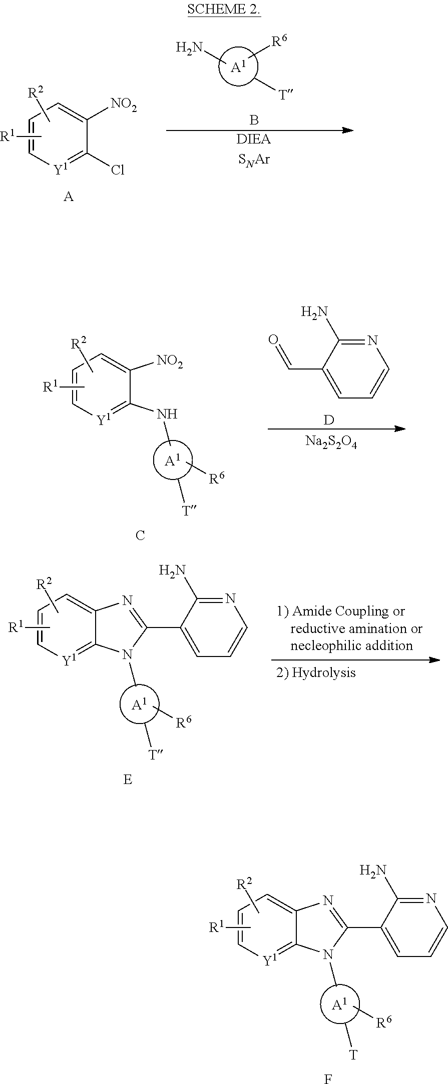

Scheme 3 illustrates a general method for preparing aminopyridinyl imidazo[4,5-b]pyridine compounds. Reaction of A with B provides C. Reaction of C with aldehyde D, and Boc deprotection provides imidazo[4,5-b]pyridine E, which is then converted to the final aminopyridinyl imidazo[4,5-b]pyridine compound F, after optional hydrolysis.

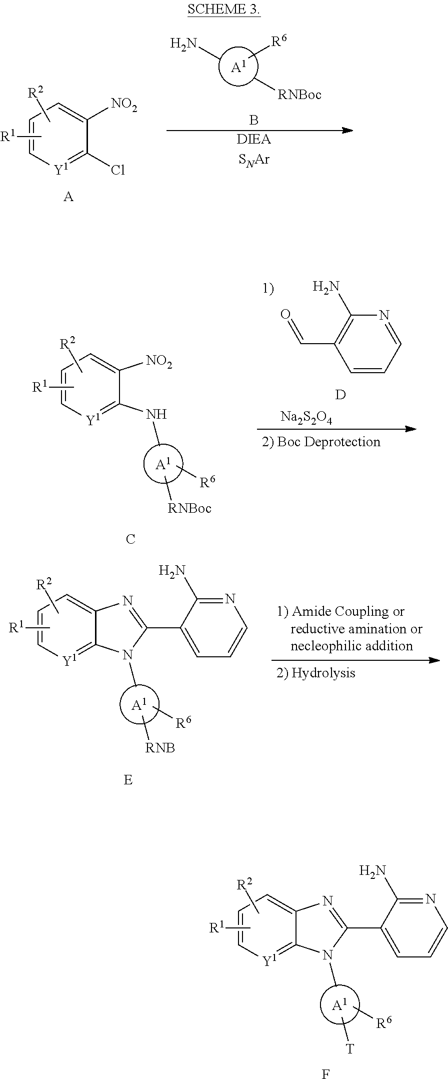

Scheme 4 illustrates a general method for preparing aminopyridinyl imidazo[4,5-b]pyridine compounds. Reaction of A with B provides C, which is then converted to D. Reaction of D with aldehyde E, and after optional hydrolysis, the final imidazo[4,5-b]pyridine compound F is afforded.

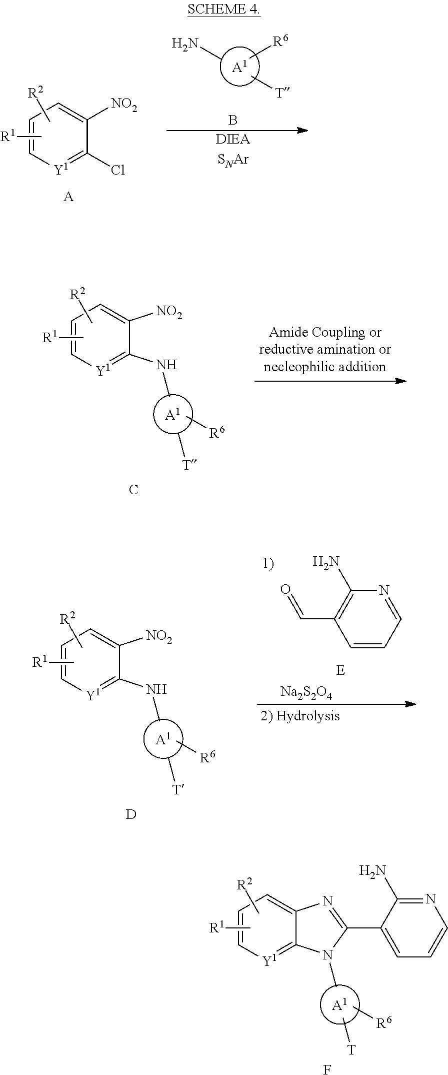

Scheme 5 illustrates a general method for preparing aminopyridinyl imidazo[4,5-b]pyridine compounds. Reaction of A with B provides C. Nitro C is reduced to provide amine D. Reaction of D with aldehyde E, and after optional hydrolysis, the final imidazo[4,5-b]pyri dine compound F is afforded.

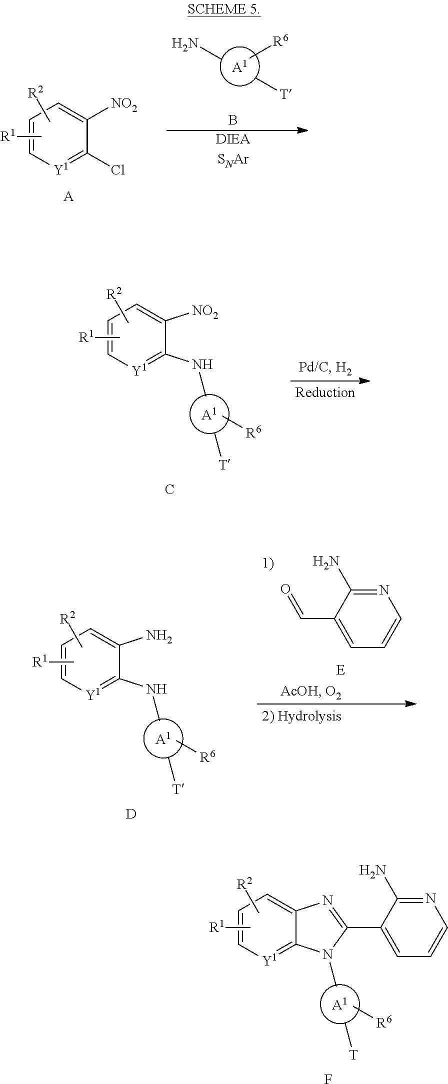

Scheme 6 illustrates a general method for preparing aminopyridinyl imidazo[4,5-b]pyridine compounds. Reaction of A with B provides C. Suzuki coupling of boronate D to C provides E, which is then converted to F. Nitro F is reduced to provide amine G. Reaction of G with aldehyde H, and after optional hydrolysis, the final imidazo[4,5-b]pyridine compound I is afforded.

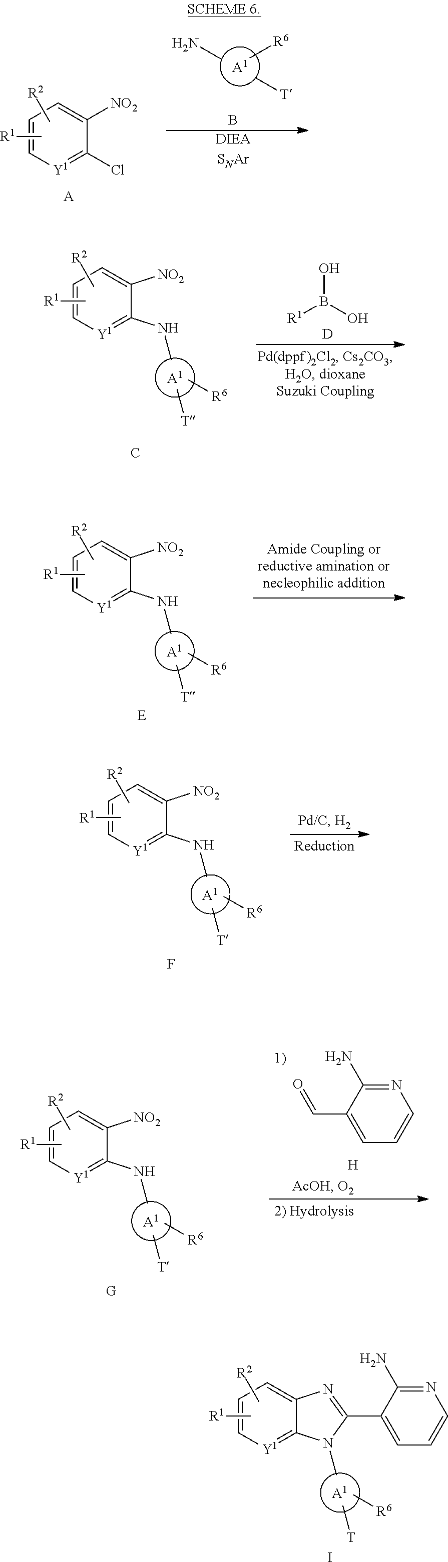

In the schemes (Scheme 1-Scheme 6) described above, T is

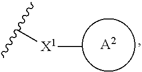

wherein “ [symbol] ” represents a bond to A¹, T′ and T″ refer to earlier stages in synthesis of T and Y¹, R¹, R², R⁶, A¹, X¹, and A² are as defined herein. In the schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference. The modular synthetic routes illustrated in the schemes can also be readily modified by one of skill in the art to provide additional compounds by conducting functional group transformations on the intermediate and final compounds. Such functional group transformations are well known in the art, as described in, for example. “Comprehensive Organic Synthesis” (B. M. Trost & I. Fleming, eds., 1991-1992).

### II. Therapeutic Applications

Another aspect of the invention provides a method of treating a disease or disorder associated with aberrant AKT1 signaling, wherein the method comprises administering to a subject in need thereof a therapeutically effective amount of a compound described herein, such as a compound of Formula I or Formula II, to treat the disease or disorder associated with aberrant AKT1 signaling. In certain embodiments, the particular compound of Formula I or Formula II is a compound defined by one of the embodiments described in Section I, above. In certain embodiments, the disease or disorder associated with aberrant AKT1 signaling is a disease or disorder associated with an AKT1 E17K genetic mutation.

Another aspect of the invention provides a method of treating cancer, wherein the method comprises administering to a subject in need thereof a therapeutically effective amount of a compound described herein, such as a compound of Formula I or Formula II, to treat the cancer. In certain embodiments, the particular compound of Formula I or Formula II is a compound defined by one of the embodiments described in Section 1, above.

Methods described herein may be further defined according to additional features, such as the identity of the cancer and/or the subject.

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, leukemia, urothelial cancer, colorectal cancer, or glioblastoma multiforme.

In certain embodiments, the cancer is a solid tumor.

In certain embodiments, the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, uterine corpus endometrial carcinoma, cervical cancer, endocervical cancer, thyroid carcinoma, prostate adenocarcinoma, skin cutaneous melanoma, bladder urothelial carcinoma, head and neck squamous cell carcinoma, or stomach adenocarcinoma.

In certain embodiments, the cancer is an adenocarcinoma, squamous cell carcinoma, epithelial neoplasm, glioma, ductal neoplasm, lobular neoplasm, cystic neoplasm, mucinous neoplasm, or serous neoplasm, acinar cell neoplasm, basal cell neoplasm, fibroepithelial neoplasm, transitional cell papilloma, or transitional cell carcinoma.

In certain embodiments, the cancer is a cervical cancer, uterine cancer, breast cancer, thyroid cancer, prostate cancer, lung cancer, bladder cancer, skin cancer, stomach cancer, lymphoma, or leukemia.

In certain embodiments, the cancer is a lymphoma or leukemia.

In certain embodiments, the cancer has active PI3K signaling. In certain embodiments, the cancer has one or more mutations in phosphatase and tensin homolog (PTEN), phosphatidylinositol-4,5-bisphosphate 3-kinase catalytic subunit alpha (PIK3CA), phosphatidylinositol-4,5-bisphosphate 3-kinase catalytic subunit beta (PIK3CB), phosphatidylinositol-4,5-bisphosphate 3-kinase catalytic subunit delta (PIK3CD), mechanistic target of rapamycin kinase (mTOR), RPTOR independent companion of mTOR complex 2 (RICTOR), MAPK associated protein 1 (MAPKAP1), or 3-phosphoinositide dependent protein kinase 1 (PDPK1), or combinations thereof.

In certain embodiments, the cancer has an AKT1 mutation. In certain embodiments, the cancer has an AKT1 E17K mutation.

Exemplary cancers reported in the literature having an AKT1 E17K mutation include breast invasive carcinoma, uterine corpus endometrial carcinoma, colon adenocarcinoma, cervical and endocervical cancer, thyroid carcinoma, lung adenocarcinoma, prostate adenocarcinoma, skin cutaneous melanoma, bladder urothelial carcinoma, head and neck squamous cell carcinoma, and stomach adenocarcinoma.

Additionally, the AKT1 E17K mutation has been in reported in many sub-types of breast cancer including ductal, lobular, and in many combinations of HER2, estrogen receptor and progesterone receptor positivity. See, for example, BMC Cancer volume 16, Article number: 622 (2016)).

In certain embodiments, the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, or metastatic melanoma. In certain embodiments, the cancer is a melanoma.

In certain embodiments, the disorder is a cancer selected from the group consisting of ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, and leukemia.

In certain embodiments, the cancer is a solid tumor. In certain embodiments, the cancer is a sarcoma or carcinoma. In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia.

In certain embodiments, the cancer is prostate cancer, breast cancer, lung cancer, liver cancer, bladder cancer, urinary tract cancer, or eye cancer. In certain embodiments, the cancer is prostate cancer. In certain embodiments, the cancer is breast cancer. In certain embodiments, the cancer is lung cancer. In certain embodiments, the cancer is liver cancer. In certain embodiments, the cancer is bladder cancer. In certain embodiments, the cancer is urinary tract cancer. In certain embodiments, the cancer is eye cancer.

In certain embodiments, the cancer is squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas (e.g., Burkitt's lymphoma and Non-Hodgkin's lymphoma); benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

In certain embodiments, the cancer is a neuroblastoma, craniopharyngioma, glioma, glioblastoma, schwannoma, astrocytoma, oligodendroglioma, medulloblastoma, pinealoma, hemangioblastoma, retinoblastoma, ependymoma, chordoma, meningioma, medullary carcinoma, small cell lung carcinoma, papillary adenocarcinoma, papillary carcinoma, mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct and gallbladder cancers, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

In certain embodiments, the cancer is a lymphoma. In certain embodiments, the cancer is Burkitt's lymphoma, diffuse large B-cell lymphoma (DLBCL), follicular lymphoma, non-Hodgkin's lymphoma, lymphoid malignancies of T-cell or B-cell origin, peripheral T-cell lymphoma, adult T-cell leukemia-lymphoma, or Waldenstrom's macroglobulinemia.

In certain embodiments, the cancer is a leukemia. In certain embodiments, the cancer is acute leukemia, lymphoblastic leukemia, acute lymphoblastic leukemia, myelogenous leukemia, acute myelogenous leukemia, acute T-cell leukemia, chronic leukemia, chronic lymphocytic leukemia, chronic myelocytic leukemia, chronic myelogenous leukemia, polycythemia vera, multiple myeloma, or erythroleukemia.

In certain embodiments, the cancer is a myelodysplastic and/or myeloproliferative syndrome. In certain embodiments, the cancer is a myelodysplastic syndrome. In certain embodiments, the cancer is a myeloproliferative syndrome.

In certain embodiments, the cancer is a cancer or related myeloproliferative disorder selected from histiocytosis, essential thrombocythemia, myelofibrosis, heavy chain disease, and other malignancies and hyperproliferative disorders of the bladder, breast, colon, lung, ovaries, pancreas, prostate, skin and uterus.

In certain embodiments, the cancer is a B-cell non-Hodgkin's lymphoma, advanced solid tumor, soft tissue sarcoma, INI1-deficient cancer, BAPI-deficient cancer, follicular lymphoma, relapsed/refractory follicular lymphoma, diffuse large B-cell lymphoma, relapsed/refractory diffuse large B-cell lymphoma, non-Hodgkin's lymphoma, pediatric non-Hodgkin's lymphoma, pediatric non-Hodgkin's lymphoma with EZH2, SMARCB1, or SMARCA4 mutation, histiocytic disorder, pediatric histiocytic disorder, pediatric histiocytic disorder with EZH2, SMARCB1, or SMARCA4 mutation, solid tumor with EZH2, SMARCB1, or SMARCA4 mutation, resistant prostate cancer, relapsed/refractory small-cell lung carcinoma, B-cell lymphoma, relapsed/refractory B-cell lymphoma, adult T-cell leukemia-lymphoma, or advanced diffuse large B-cell lymphoma.

In certain embodiments, the cancer is a malignant rhabdoid tumor, atypical teratoid rhabdoid tumor, epithelioid sarcoma, renal medullary carcinoma, pancreatic undifferentiated rhabdoid carcinoma, schwannoma, epithelioid malignant peripheral nerve sheath tumor, or diffuse intrinsic glioma.

In certain embodiments, the cancer is retinoblastoma multiforme, metastatic castration-resistant prostate cancer, prostate small cell neuroendocrine carcinoma, small-cell lung cancer, triple-negative breast cancer, hepatocellular carcinoma, bladder cancer, or urinary tract cancer.

In certain embodiments, the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma. In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C& D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is a metastatic cancer. In certain embodiments, the cancer is a relapsed and/or refractory cancer.

In certain embodiments, the cancer is ovarian cancer, uterine cancer, gestational trophoblastic disease, endometrial cancer, cervical cancer, embryonal carcinoma, choriocarcinoma, prostate cancer (including hormone insensitive and castrate resistant prostate cancers), testicular tumors (including germ cell testicular cancer/seminoma), cystadenocarcinoma, breast cancer (including estrogen-receptor positive breast cancer), brain tumors (including neuroblastoma, craniopharyngioma, glioma, glioblastoma, schwannoma, astrocytoma, oligodendroglioma, medulloblastoma, and pinealoma), hemangioblastoma, retinoblastoma, ependymoma, chordoma, meningioma, medullary carcinoma, lung cancer (including small cell lung carcinoma, papillary adenocarcinomas, and papillary carcinoma), mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct and gallbladder cancers, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

Methods of Treating Diseases or Disorders Associated with Active PI3K Signaling

A method of treating a disease or disorder associated with active PI3K signaling. The method comprises administering to a subject in need thereof a therapeutically effective amount of a compound described herein, such as a compound of Formula I or Formula II, to treat the disease or disorder. In certain embodiments, the disease or disorder features hyperactive PI3K signaling.

### Subjects

In certain embodiments, the subject is a human. In certain embodiments, the subject is an adult human. In certain embodiments, the subject is a pediatric human.

### Methods of Inhibiting AKT1 Acclivity

Another aspect of the invention provides a method of inhibiting AKT1 activity. The method comprises contacting an AKT1 with an effective amount of a compound described herein, such as a compound of Formula I or Formula II, to thereby inhibit the AKT1 activity.

In certain embodiments, the AKT1 is an AKT1 E17K. In certain embodiments, the AKT1 protein is AKT1 E17K.

### Medical Uses

Another aspect of the invention provides for the use of a compound described herein (such as a compound of Formula I or Formula II, or other compounds in Section I) in the manufacture of a medicament. In certain embodiments, the medicament is for treating a disorder described herein, such as cancer.

Another aspect of the invention provides for the use of a compound described herein (such as a compound of Formula I or Formula II, or other compounds in Section I) for treating a medical disorder, such as a medical disorder described herein, such as cancer.

### IV. Combination Therapy

Another aspect of the invention provides for combination therapy compounds described herein (such as a compound of Formula I or Formula II, or other compounds in Section I) or their stereoisomer or pharmaceutically acceptable salts may be used in combination with additional therapeutic agents to treat medical disorders, such as an autoimmune disorder or a cancer.

In certain embodiments, the present invention provides a method of treating a disclosed disease or condition comprising administering to a patient in need thereof an effective amount of a compound disclosed herein or a stereoisomer or pharmaceutically acceptable salt thereof and co-administering simultaneously or sequentially an effective amount of one or more additional therapeutic agents, such as those described herein. In certain embodiments, the method includes co-administering one additional therapeutic agent. In certain embodiments, the method includes co-administering two additional therapeutic agents. In certain embodiments, the combination of the disclosed compound and the additional therapeutic agent or agents acts synergistically.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In certain embodiments, one or more other therapeutic agent and a compound or composition of the invention are administered as a multiple dosage regimen more than 24 hours apart.

### Anti-Cancer Agents

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264 Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor ipilumumab has been approved by the United States Food and Drug Administration for treating melanoma. In certain embodiments, the immune checkpoint inhibitor comprises pembrolizumab.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Accordingly, another aspect of the invention provides a method of treating cancer in a patient, where the method comprises administering to the patient in need thereof (i) a therapeutically effective amount of a compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-h]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, R^{X}-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a NITHI Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

In certain embodiments, the second anti-cancer agent is a hormone therapy agent. Exemplary hormone therapy agents include, for example, fulvestrant (faslodex) and other agents that target estrogen receptor and estrogen signaling. In certain embodiments, the second therapeutic agent is a CDK4/6 inhibitor.

### Additional Considerations

The doses and dosage regimen of the active ingredients used in the combination therapy may be determined by an attending clinician. In certain embodiments, the compound described herein (such as a compound of Formula I or Formula II, or other compounds in Section I) and the additional therapeutic agent(s) are administered in doses commonly employed when such agents are used as monotherapy for treating the disorder. In other embodiments, the compound described herein (such as a compound of Formula I or Formula II, or other compounds in Section I) and the additional therapeutic agent(s) are administered in doses lower than the doses commonly employed when such agents are used as monotherapy for treating the disorder. In certain embodiments, the compound described herein (such as a compound of Formula I or Formula II, or other compounds in Section I) and the additional therapeutic agent(s) are present in the same composition, which is suitable for oral administration.

In certain embodiments, the compound described herein (such as a compound of Formula I or Formula II, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the compound described herein (such as a compound of Formula I or Formula I, or other compounds in Section I), a pharmaceutically acceptable carrier, vehicle or diluent, and optionally at least one additional therapeutic agent listed above.

### III. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following. (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (such as a compound of Formula I or Formula II, or other compounds in Section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof, (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a compound described herein in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and is not intended to limit the invention.

### Abbreviations

¹H NMR Proton nuclear magnetic resonance spectroscopy
ACN Acetonitrile
AcOH Acetic acid
Al₂O₃ Aluminum oxide
aq. aqueous
Ar Argon (gas)
BH₃·Me₂S Borane dimethyl sulfide complex
BINAP 2,2′-bis(diphenylphosphino)-1,1′-binaphthyl
BnBr Benzyl bromide
Boc₂0 Di-tert-butyl dicarbonate
calcd. calculated
CbzCl Benzyl chloroformate
CDCl₃ Deuterated chloroform
CH₃OCH₂PPh₃Cl chloridotris(triphenylphosphene)rhodium(I); Wilkinson's catalyst
CO₂ Carbon dioxide
(COCl)₂ Oxalyl chloride
CO(OCCl₃)₂ Triphosgene
Cs₂CO₃ Caesium carbonate
DCE 1,2-Dichloroethane
DCM Dichloromethane
DIBAL-H Diisobutylaluminium hydride
DIEA N,N-Diisopropylethylamine
DMA Dimethylacetamide
DMF N,N-Dimethylformamide
DMP Dess-Martin periodinane
DMSO Dimethylsulfoxide
DMSO-d₆ Hexadeuterodimethyl sulfoxide
EA Ethyl acetate
EDCI 1-(3-Dimethylaminopropyl)-3-ethylcarbodiimide hydrochloride
EtOAc Ethyl acetate
EtOH Ethanol
FA Formic acid
Fe Iron
H₂ Hydrogen (gas)
HATU N-[(dimethylamino)-3-oxo-1H-1,2,3-triazolo[4,5-b]pyridin-1-yl-methylene]-N-methylmethanaminium hexafluorophosphate
HCl Hydrochloric acid
H₂O Water
H₂O₂ Hydrogen peroxide
HOAc Acetic acid
HPLC High performance liquid chromatography
hr hour
hrs hours
i-PrMgCl Isopropylmagnesium chloride
i-PrOH Isopropyl alcohol
KF Potassium fluoride
LCMS Liquid Chromatography Mass Spectrometry
LDA Lithium diisopropylamide
LiAlH₄ Lithium aluminum hydride
LiCl Lithium chloride
LiOH Lithium hydroxide
m-CPBA 3-Chloroperbenzoic acid
MeI Iodomethane
MeOD Tetradeuteromethanol
MeOH Methanol
MePPh₃Br Methyltriphenylphosphonium bromide
min minute(s)
MnO₂ Manganese dioxide
MS Mass spectrometry
MTBE Methyl tert-butyl ether
N₂ Nitrogen (gas)
NaBH(OAc)s Sodium triacetoxyborohydride
NaBH₃CN Sodium cyanoborohydride
NaClO Sodium hypochlorite
NaH Sodium hydride
NaHCO₃Sodium bicarbonate
NaHMDS Sodium bis(trimethylsilyl)amide
NaI Sodium iodide
NaOH Sodium hydroxide
Na₂CO₃ Sodium carbonate
Na₂SO₄ Sodium sulfate
Na₂S₂O₃ Sodium thiosulfate
Na₂S₂O₄ Sodium dithionite
NBS N-Bromosuccinimide
NH₂Boc Tert-butyl carbamate
NH₄C Ammonium chloride
NH₄HCO₃ Ammonium bicarbonate
NMP N-Methyl-2-Pyrrolidone
O₂ Oxygen (gas)
PBr₃ Phosphorus tribromide
Pd Palladium
Pd₂(dba)₃ Tris(dibenzylideneacetone)dipalladium Pd/C Palladium on carbon
Pd(dppf)Cl₂ [1,1′-Bis(diphenylphosphino)ferrocene]dichloropalladium(II)
Pd(OAc)₂ Palladium(II) acetate
Pd(OH)₂ Palladium(H) hydroxide
PE Petroleum ether
PPh₃ Triphenylphosphine
prep-HPLC Preparative high performance liquid chromatography
psi Pound per square inch
py Pyridine
RT Room temperature
Rₜ Retention time
sat. saturated
SFC Supercritical fluid chromatography
SOCl₂ Thionyl chloride
TBAF Tetrabutylammonium fluoride
t-BuONa Sodium tert-butoxide
TEA Triethylamine
TFA Trifluoroacetic acid
TFP Tri(2-furyl)phosphine
THF Tetrahydrofuran
TBSCl Tert-Butyldimethylsilyl chloride
TMSCl Trimethylsilyl chloride
TMSOK Potassium trimethylsilanolate
Zn Zinc
ZnCl₂ Zinc chloride

### General HPLC Methods

5-95AB_1 min_220&254_Agilent (LCMS)

Mobile Phase: 1.5 mL×4 L TFA in water (solvent A) and 0.75 mL×4 L TFA in acetonitrile (solvent B), using the elution gradient 5%-95% (solvent B) over 0.4 minutes and holding at 95% for 0.3 minutes at a flow rate of 2.0 mL/min. Column: Agilent Poroshell 120 EC-C18 2.7 um 3.0*30 mm; Wavelength: UV 220 nm & 254 nm: Column temperature: 50° C.; MS ionization: ESI.

### 5-95AB_1.5MIN_220&254_Shimadzu (LCMS)

Mobile Phase: 2.75 mL×4 L TFA in water (solvent A) and 2.5 mL×4 L TFA in acetonitrile (solvent B), using the elution gradient 10%-80% (solvent B) over 6 minutes and holding at 80% for 2 minutes at a flow rate of 1.2 mL/min. Column: WelCh Ultimate® XB-C18 3.0*50 mm, 3 um; Wavelength: UV 220 nm, 215 nm, & 254 nm; Column temperature: 40° C.

### 10-80AB_4MIN_220&254_Shimadzu (LCMS)

Mobile Phase: 1.5 mL×4 L TFA in water (solvent A) and 0.75 mL×4 L TFA in acetonitrile (solvent B), using the elution gradient 10%-80% (solvent B) over 3 minutes and holding at 80% for 0.5 minutes at a flow rate of 0.8 mL/min. Column: Nano Chrom 120 C18 3.0*30 mm, 3 um; Wavelength: UV 220 nm & 254 nm; Column temperature: 50° C.; MS ionization: ESI.

### 30-90AB_4MIN_220&254_Shimadzu (LCMS)

Mobile Phase: 1.5 mL×4 L TFA in water (solvent A) and 0.75 mL×4 L TFA in acetonitrile (solvent B), using the elution gradient 300%-90% (solvent B) over 3 minutes and holding at 90% for 0.5 minutes at a flow rate of 0.8 mL/min. Column: SHIMADZU ShimNex HE C18-AQ 3 um, 3.0*30 mm; Wavelength: UV 220 nm & 254 nm. Column temperature: 50° C.; MS ionization: ESI: Detector: PDA.

### 10-80AB_8MIN (HPLC)

Mobile Phase: 2.75 mL×4 L TFA in water (solvent A) and 2.5 mL×4 L TFA in acetonitrile (solvent B), using the elution gradient 10%-80% (solvent B) over 6 minutes and holding at 80% for 2 minutes at a flow rate of 1.2 mL/min. Column: WelCh Ultimate® XB-C18 3.0*50 mm, 3 um; Wavelength. UV 220 nm, 215 nm, & 254 nm; Column temperature: 40° C.

### Example 1. Synthesis of 3-((4-(2-(2-aminopyridin-3-yl)-5-phenyl-3H-imidazo[4,5-b]pyridin-3-yl)phenyl)carbamoyl)cyclopentane-1-carboxylic acid (Compound I-1)

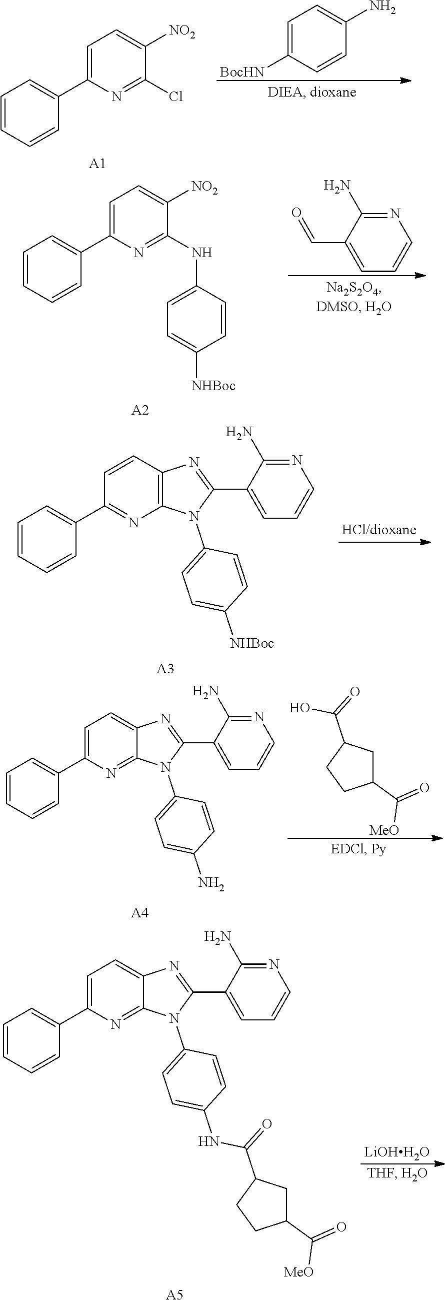

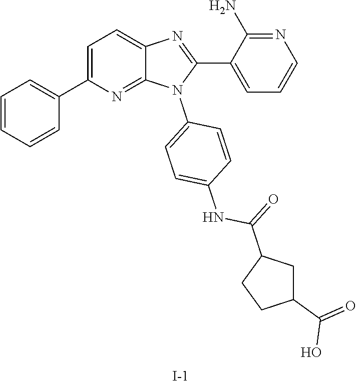 [SMILES: Nc1ncccc1-c1nc2ccc(-c3ccccc3)nc2n1-c1ccc(NC(=O)C2CCC(C(=O)O)C2)cc1 ; [I-]]

Step 1. To a solution of 2-chloro-3-nitro-6-phenylpyridine A1 (444 mg, 2.13 mmol) in 1,4-dioxane (10.0 mL), DIEA (345 mg, 3.41 mmol) and tert-butyl (4-aminophenyl)carbamate (500 mg, 2.13 mmol) were added and the mixture was stirred at 80° C. for 12 hrs. The reaction mixture was then diluted with water (50 mL) and extracted with ethyl acetate (200 mL×3). The organic layers were combined, washed with saturated aqueous NaHCO₃ (100 mL×2), brine (200 mL×5), and the organic layers were dried over Na₂SO₄, filtered and concentrated in vacuo to afford tert-butyl (4-((3-nitro-6-phenylpyridin-2-yl)amino)phenyl)carbamate A2 (800 mg, 92.3% yield) as a brown solid. ¹H NMR (400 MHz, DMSO-d₆) β_{H}=10.05 (s, 1H), 9.39 (s, 1H), 8.59 (d, J=8.8 Hz, 1H), 8.13-8.03 (m, 2H), 7.66-7.46 (m, 8H), 1.49 (s, 9H).

Step 2. To a solution of tert-butyl (4-((3-nitro-6-phenylpyridin-2-yl)amino)phenyl)carbamate A2 (700 mg, 1.72 mmol) and 2-aminopyridine-3-carbaldehyde (210 mg, 1.72 mmol) in DMSO (14.0 mL) and methanol (14.0 mL), Na₂S₂O₄ (0.75 g, 4.31 mmol) was added, and the mixture was stirred at 100° C. for 18 hrs. The reaction mixture was concentrated to dryness, water (30 mL) was added, and the resulting mixture was extracted with EtOAc (50 mL×3). The combined organic phase was washed with saturated LiCl aqueous (80 mL×3), dried over anhydrous Na₂SO₄, filtered and concentrated to dryness. The crude product was purified by Combi-Flash column (EtOAc in PE, 0-41%) to afford tert-butyl (4-(2-(2-aminopyridin-3-yl)-5-phenyl-3H-imidazo[4,5-b]pyridin-3-yl)phenyl)carbamate A3 (450 mg, 53.2% yield) as a yellow solid. ¹H NMR (400 MHz, DMSO-d₆) δ_{H}=9.65 (s, 1H), 8.25 (d, J=8.4 Hz, 1H), 8.10-7.94 (m, 4H), 7.62 (d, J=8.8 Hz, 2H), 7.50-7.43 (m, 2H), 7.42-7.35 (m, 3H), 7.23 (dd, J=2.0, 8.0 Hz, 1H), 7.06 (s, 2H), 6.43 (dd, J=4.8, 7.6 Hz, 1H), 1.50 (s, 9H).

Step 3. A solution of tert-butyl (4-(2-(2-aminopyridin-3-yl)-5-phenyl-3H-imidazo[4,5-b]pyridin-3-yl)phenyl)carbamate A3 (500 mg, 1.04 mmol) in HCl/dioxane (6.00 mL, 4.0 M) was stirred at 25° C. for 2 hrs. The reaction mixture was concentrated directly to afford 3-(3-(4-aminophenyl)-5-phenyl-3H-imidazo[4,5-b]pyridin-2-yl)pyridin-2-amine A4 (450 mg, crude) as a yellow oil. ¹H NMR (400 MHz, DMSO-d₆) δ_{H}=8.54-8.39 (m, 1H), 8.35 (d, J=8.4 Hz, 1H), 8.13 (dd, J=1.6, 6.0 Hz, 1H), 8.07-8.04 (m, 3H), 7.91 (dd, J=1.6, 7.6 Hz, 1H), 7.54-7.36 (m, 6H), 7.20-7.08 (m, 2H), 6.91 (dd, J=6.4, 7.2 Hz, 1H).

Step 4. To a stirred solution of 3-methoxycarbonylcyclopentanecarboxylic acid (45.0 mg, 0.260 mmol) and 3-(3-(4-aminophenyl)-5-phenyl-3H-imidazo[4,5-b]pyridin-2-yl)pyridin-2-amine A4 (100 mg, 0.260 mmol) in pyridine (2.00 mL), EDCI (56.0 mg, 0.290 mmol) was added and the reaction mixture was stirred at 25° C., under N₂, for 16 hrs. The reaction mixture was quenched with water (5 mL) and extracted with EtOAc (20 mL). The organic layer was washed with 1 M HCl (10 mL×2), brine (20 mL 2), dried over anhydrous sodium sulfate, filtered and the filtrate was concentrated and purified by flash chromatography on silica gel (95% of EtOAc in PE) to afford methyl 3-((4-(2-(2-aminopyridin-3-yl)-5-phenyl-3H-imidazo[4,5-b]pyridin-3-yl)phenyl)carbamoyl)cyclopentane-1-carboxylate A5 (70.0 mg, 49.7% yield) as a yellow solid. ¹H NMR (400 MHz, DMSO-d₆) δ_{H}=8.74 (s, 1H), 8.20-7.90 (m, 4H), 7.80 (dd, J=1.6, 8.8 Hz, 3H), 7.50-7.31 (m, 5H), 7.19 (dd, J=1.6, 7.6 Hz, 1H), 6.62 (s, 2H), 6.42 (dd, J=4.8, 7.6 Hz, 1H), 3.77 (s, 3H), 3.12-2.89 (m, 2H), 2.37-2.06 (m, 5H), 2.04-1.98 (m, 1H).

Step 5. To a stirred solution of methyl 3-((4-(2-(2-aminopyridin-3-yl)-5-phenyl-3H-imidazo[4,5-b]pyridin-3-yl)phenyl)carbamoyl)cyclopentane-1-carboxylate A5 (70.0 mg, 0.130 mmol) in THF (1.00 mL) and water (1.00 mL), LiOH·H₂O (11.0 mg, 0.260 mmol) was added and the reaction mixture was stirred at 25° C. under N₂ for 16 hrs. The reaction mixture was adjusted to pH ~5 with 1 M HCl and subsequently concentrated to afford a residue. The crude product was purified by prep-HPLC (column: Welch Xtimate C18 150*30 mm*5 μm, method: water (FA)-ACN, begin B: 20, end B: 50) to afford 3-((4-(2-(2-aminopyridin-3-yl)-5-phenyl-3H-imidazo[4,5-b]pyridin-3-yl)phenyl)carbamoyl)cyclopentane-1-carboxylic acid 1-1 (19.0 mg, 26.3% yield) as a white solid. ¹H NMR (400 MHz, DMSO-d₆) δ_{H}=12.16 (s, 1H), 10.19 (s, 1H), 8.25 (d, J=8.4 Hz, 1H), 8.10-7.92 (m, 4H), 7.78 (d, J=8.8 Hz, 2H), 7.53-7.34 (m, 5H), 7.24 (dd, J=1.6, 7.6 Hz, 1H), 7.05 (s, 2H), 6.44 (dd, J=4.8, 7.6 Hz, 1H), 2.99-2.69 (m, 2H), 2.25-2.09 (m, 1H), 2.05-1.72 (m, 5H).

### Example 2. Synthesis of trans-4-[[4-[2-(2-amino-3-pyridyl)-6-phenyl-benzimidazol-1-yl]phenyl]carbamoyl]cyclohexanecarboxylic acid (Compound I-2)

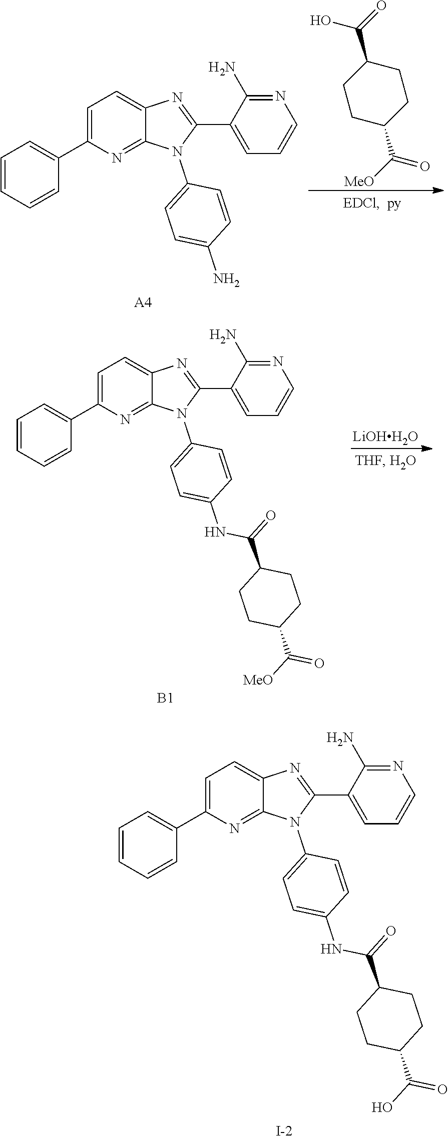

Step 1. To a stirred solution of 3-(3-(4-aminophenyl)-5-phenyl-3H-imidazo[4,5-b]pyridin-2-yl)pyridin-2-amine A4 (100 mg, 0.260 mmol) and trans-4-methoxycarbonylcyclohexanecarboxylic acid (49.0 mg, 0.260 mmol) in pyridine (2.00 mL), EDCI (56.0 mg, 0.290 mmol) was added and the reaction mixture was stirred at 25° C. under N₂ for 16 hrs. The reaction mixture was quenched with water (5 mL) and extracted with EtOAc (20 mL). The organic layer was washed with 1 M HCl (10 mL×2), brine (20 mL×2), dried over anhydrous sodium sulfate, filtered and the filtrate was concentrated. The residue was purified by flash chromatography on silica gel (95% of EtOAc in PE) to afford methyl (1r,4r)-4-((4-(2-(2-aminopyridin-3-yl)-5-phenyl-3H-imidazo[4,5-b]pyridin-3-yl)phenyl)carbamoyl)cyclohexane-1-carboxylate BI (70.0 mg, 48.5% yield) as a yellow solid. ¹H NMR (400 MHz, DMSO-d₆) δ_{H}=8.19-7.97 (m, 4H), 7.80 (d, J=8.4 Hz, 1H), 7.72 (d, J=8.8 Hz, 2H), 7.47-7.29 (m, 6H), 7.16 (dd, J=2.0, 8.0 Hz, 1H), 6.61 (s, 2H), 6.41 (dd, J=4.8, 7.6 Hz, 1H), 3.70 (s, 3H), 2.44-2.06 (m, 6H), 1.74-1.64 (m, 2H), 1.54-1.46 (m, 2H).

Step 2. To a stirred solution of methyl (1r,4r)-4-((4-(2-(2-aminopyridin-3-yl)-5-phenyl-3H-imidazo[4,5-b]pyridin-3-yl)phenyl)carbamoyl)cyclohexane-1-carboxylate B1 (60.0 mg, 0.110 mmol) in THF (1.00 mL) and water (1.00 mL), LiOH·H₂O (8.80 mg, 0.220 mmol) was added and the reaction mixture was stirred at 25° C. under N₂ for 16 hrs. The reaction mixture was adjusted to pH ~5 with 1M HCl and subsequently concentrated to afford a residue. The crude product was purified by prep-HPLC (column. Welch Xtimate C18 150*30 mm*5 μm, method: water (FA)-ACN, begin B: 23, end B: 53) to afford trans-4-[[4-[2-(2-amino-3-pyridyl)-6-phenyl-benzimidazol-1-yl]phenyl]carbamoyl]cyclohexanecarboxylic acid 1-2 (33.0 mg, 55.8% yield) as a white solid. ¹H NMR (400 MHz, DMSO-d₆) δ_{H}=12.17 (s, 1H), 10.12 (s, 1H), 8.25 (d, J=8.4 Hz, 1H), 8.08-7.93 (m, 4H), 7.78 (d, J=8.8 Hz, 2H), 7.51-7.34 (m, 5H), 7.24 (dd, J=2.0, 7.6 Hz, 1H), 7.03 (s, 2H), 6.44 (dd, J=4.8, 7.6 Hz, 1H), 2.42-2.13 (m, 2H), 2.08-1.83 (m, 4H), 1.61-1.26 (m, 4H).

### Example 3. Synthesis of cis-4-[[4-[2-(2-amino-3-pyridyl)-5-phenyl-imidazo[4,5-b]pyridin-3-yl]phenyl]carbamoyl]cyclohexanecarboxylic acid (Compound I-3)

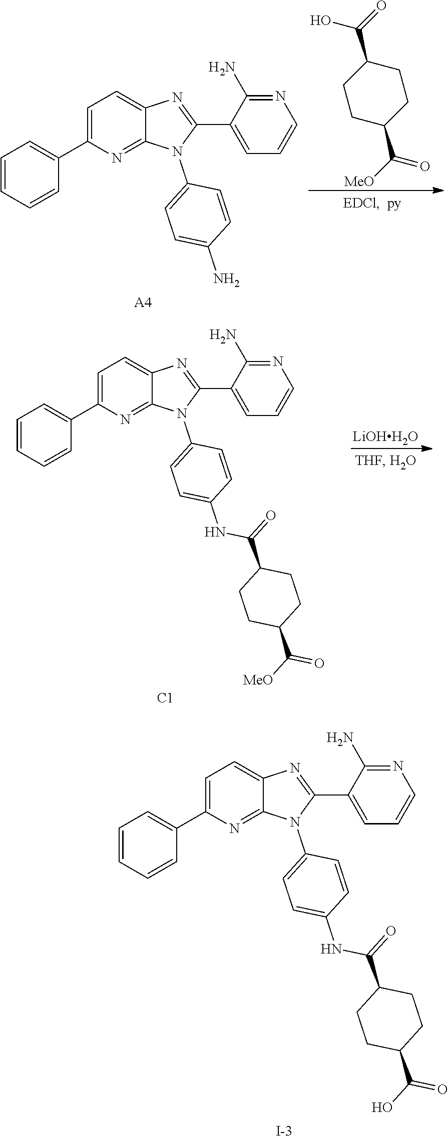

Step 1. To a stirred solution of 3-(3-(4-aminophenyl)-5-phenyl-3H-imidazo[4,5-b]pyridin-2-yl)pyridin-2-amine A4 (100 mg, 0.260 mmol) and cis-4-methoxycarbonylcyclohexanecarboxylic acid (49.0 mg, 0.260 mmol) in pyridine (2.00 mL), EDCI (56.0 mg, 0.290 mmol) was added, and the reaction mixture was stirred at 25° C. under N₂ for 16 hrs. The reaction mixture was quenched with water (5 mL) and extracted with EtOAc (20 mL). The organic layer was washed with 1 M HCl (10 mL×2), brine (20 mL×2), dried over anhydrous sodium sulfate, filtered and the filtrate was concentrated. The residue was purified by flash chromatography on silica gel (95%, of EtOAc in PE) to afford methyl (1s,4s)-4-((4-(2-(2-aminopyridin-3-yl)-5-phenyl-3H-imidazo[4,5-b]pyridin-3-yl)phenyl)carbamoyl)cyclohexane-1-carboxylate C1 (70.0 mg, 48.5% yield) as a yellow solid. LCMS Rₜ=0.51 min in 1.0 min chromatography, purity 98.8%, MS ESI calcd for 546.24 [M+H]⁺ 547.24, found 547.4.

Step 2. To a stirred solution of methyl (1s,4s)-4-((4-(2-(2-aminopyridin-3-yl)-5-phenyl-3H-imidazo[4,5-b]pyridin-3-yl)phenyl)carbamoyl)cyclohexane-1-carboxylate C1 (60.0 mg, 0.110 mmol) in THF (1.00 mL) and water (1.00 mL), LiOH·H₂O (8.80 mg, 0.220 mmol) was added and the reaction mixture was stirred at 25° C. under N₂ for 16 hrs. The reaction mixture was adjusted to pH ~5 with 1 M HCl and subsequently concentrated to afford a residue. The crude product was purified by prep-HPLC (column: Welch Xtimate C18 150*30 mm*5 μm, method: water (FA)-ACN, begin B: 22, end B: 52) to afford cis-4-[[4-[2-(2-amino-3-pyridyl)-5-phenyl-imidazo[4,5-b]pyridin-3-yl]phenyl]carbamoyl]cyclohexanecarboxylic acid I-3 (18.0 mg, 30.1% yield) as a white solid. ¹H NMR (400 MHz, DMSO-d₆) δ_{H}=12.19 (s, 1H), 10.08 (s, 1H), 8.25 (d, J=8.4 Hz, 1H), 8.11-7.92 (m, 4H), 7.77 (d, J=8.8 Hz, 2H), 7.53-7.32 (m, 5H), 7.24 (dd, J=1.6, 7.6 Hz, 1H), 7.04 (s, 2H), 6.44 (dd, J=4.8, 7.6 Hz, 1H), 2.54 (t, J=4.4 Hz, 1H), 2.45 (s, 1H), 2.16-1.93 (m, 2H), 1.81-1.42 (m, 6H).

### Example 4. Synthesis of 3-[[4-[2-(2-amino-3-pyridyl)-5-phenyl-imidazo[4,5-b]pyridin-3-yl]phenyl]carbamoyl]cyclohexanecarboxylic acid (Compound I-4)

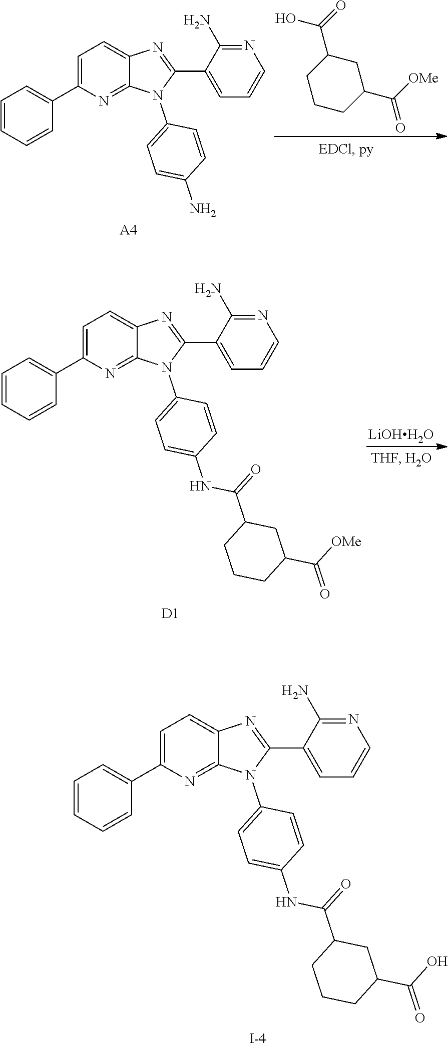

Step 1. To a stirred solution of 3-methoxycarbonylcyclohexanecarboxylic acid (49.0 mg, 0.260 mmol) and 3-(3-(4-aminophenyl)-5-phenyl-3H-imidazo[4,5-b]pyridin-2-yl)pyridin-2-amine A4 (100 mg, 0.260 mmol) in pyridine (2.00 mL), EDCI (56.0 mg, 0.290 mmol) was added and the reaction mixture was stirred at 25° C. under N₂ for 16 hrs. The reaction mixture was quenched with water (10 mL) and extracted with EtOAc (20 mL×2). The combined organic layers were washed with 1 M HCl solution (20 mL×2), brine (20 mL×2), dried over anhydrous sodium sulfate, and filtered. The filtrate was concentrated and purified by flash chromatography on silica gel (95% of EtOAc in PE) to give methyl 3-[[4-[2-(2-amino-3-pyridyl)-5-phenyl-imidazo[4,5-b]pyridin-3-yl]phenyl]carbamoyl]cyclohexanecarboxylate D1 (60.0 mg, 41.5% yield) as a yellow solid. LCMS Rₜ=0.52 min in 1.0 min chromatography, purity 77.2%, MS ESI calcd for 546.24 [M+H]⁺ 547.24, found 547.2.

Step 2. To a stirred solution of methyl 3-[[4-[2-(2-amino-3-pyridyl)-5-phenyl-imidazo[4,5-b]pyridin-3-yl]phenyl]carbamoyl]cyclohexanecarboxylate D1 (60.0 mg, 0.110 mmol) in THF (1.00 mL) and water (1.00 mL), LiOH·H₂O (10.0 mg, 0.250 mmol) was added and the reaction mixture was stirred at 25° C. under N₂ for 16 hrs. The reaction mixture was adjusted to pH ~5 with 1 M HCl and concentrated to afford a residue. The crude product was purified by prep-HPLC (column: Welch Xtimate C18 150*30 mm*5 μm, method: water (FA)-ACN, begin B: 22, end B: 52) to afford 3-[[4-[2-(2-amino-3-pyridyl)-5-phenyl-imidazo[4,5-b]pyridin-3-yl]phenyl]carbamoyl]cyclohexanecarboxylic acid I-4 (28.0 mg, 58.5% yield) as a white solid. ¹H NMR (400 MHz, DMSO-d₆) δ_{H}=12.20 (s, 1H), 10.12 (s, 1H), 8.26 (d, J=8.4 Hz, 1H), 8.09-7.92 (m, 4H), 7.78 (d, J=8.8 Hz, 2H), 7.51-7.35 (m, 5H), 7.24 (dd, J=1.6, 7.6 Hz, 1H), 7.04 (s, 2H), 6.44 (dd, J=4.8, 7.6 Hz, 1H), 2.29 (s, 2H), 2.11-1.76 (m, 4H), 1.58-1.15 (m, 4H).

### Example 5. Synthesis of 4-((4-(2-(2-aminopyridin-3-yl)-5-phenyl-3H-imidazo[4,5-b]pyridin-3-yl)-3-fluorophenyl)carbamoyl)cyclohexane-1-carboxylic acid (Compound I-5)

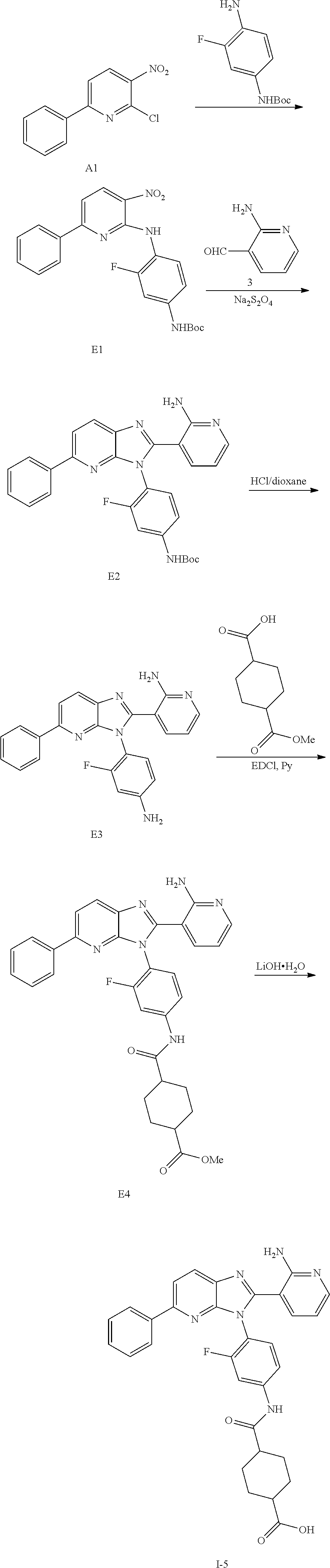

Step 1. To a stirred solution of 2-chloro-3-nitro-6-phenylpyridine A1 (800 mg, 3.41 mmol) and tert-butyl (4-amino-3-fluorophenyl)carbamate (926 mg, 4.09 mmol) in DMSO (10.0 mL), DIEA (3.10 mL, 17.0 mmol) was added and the reaction mixture was stirred at 100° C. under N₂ for 16 hrs. The reaction mixture was diluted with EtOAc (50.0 mL), washed with water (50.0 mL) and brine (100 mL×2), dried over anhydrous sodium sulfate, filtered and the filtrate was concentrated. The crude product was purified by flash chromatography on silica gel (30% of EtOAc in PE) to afford tert-butyl (3-fluoro-4-((3-nitro-6-phenylpyridin-2-yl)amino)phenyl)carbamate E1 (800 mg, 55.3% yield) as a yellow solid. ¹H NMR (400 MHz, CDCl₃) δ=10.23 (s, 1H), 8.59 (d, J=8.8 Hz, 1H), 8.23 (t, J=8.8 Hz, 1H), 8.09-7.93 (m, 2H), 7.57-7.41 (m, 4H), 7.31 (d, J=8.8 Hz, 1H), 7.10 (d, J=9.2 Hz, 1H), 6.54 (s, 1H), 1.54 (s, 9H).

Step 2. To a stirred solution of 2-aminonicotinaldehyde (322 mg, 2.64 mmol) and tert-butyl (3-fluoro-4-((3-nitro-6-phenylpyridin-2-yl)amino)phenyl)carbamate E1 (800 mg, 1.88 mmol) in DMSO (10.0 mL) and methanol (1.00 mL), Na₂S₂O₄ (1.30 g, 7.54 mmol) was added and the reaction mixture was stirred at 95° C. under N₂ for 16 hrs. The reaction mixture was diluted with EtOAc (50.0 mL), washed with water (50.0 mL) and brine (50 mL×2), dried over anhydrous sodium sulfate, filtered and the filtrate was concentrated to afford a residue. The crude product was purified by flash chromatography on silica gel (95% of EtOAc in PE) to afford tert-butyl (4-(2-(2-aminopyridin-3-yl)-5-phenyl-3H-imidazo[4,5-b]pyridin-3-yl)-3-fluorophenyl)carbamate E2 (360 mg, 38.5% yield) as a yellow solid. ¹H NMR (400 MHz, DMSO-d₆) δ=9.92 (s, 1H), 8.28 (d, J=8.4 Hz, 1H), 8.08-7.95 (m, 4H), 7.68-7.56 (m, 2H), 7.50-7.36 (m, 4H), 7.24 (dd, J=1.6, 7.6 Hz, 1H), 7.17 (s, 2H), 6.46 (dd, J=4.8, 7.6 Hz, 1H), 1.51 (s, 9H).

Step 3. To a stirred solution of tert-butyl (4-(2-(2-aminopyridin-3-yl)-5-phenyl-3H-imidazo[4,5-b]pyridin-3-yl)-3-fluorophenyl)carbamate E2 (120 mg, 0.240 mmol) in 1,4-dioxane (1.00 mL), HCl/dioxane (3.00 mL, 2 M) was added and the reaction mixture was stirred at 25° C. under N₂ for 16 hrs. The reaction mixture was concentrated to afford 3-(3-(4-amino-2-fluorophenyl)-5-phenyl-3H-imidazo[4,5-b]pyridin-2-yl)pyridin-2-amine E3 (95.0 mg, 99.2% yield) as a yellow solid. LCMS Rₜ=0.49 min in 1.0 min chromatography, purity 85.5%, MS ESI calcd. for 396.15 [M+H]⁺ 397.15, found 397.2.

Step 4. To a stirred solution of 3-(3-(4-amino-2-fluorophenyl)-5-phenyl-3H-imidazo[4,5-b]pyridin-2-yl)pyridin-2-amine E3 (100 mg, 0.250 mmol) and 4-(methoxycarbonyl)cyclohexane-1-carboxylic acid (51.0 mg, 0.250 mmol) in pyridine (2.00 mL), EDCI (58.0 mg, 0.300 mmol) was added and the reaction mixture was stirred at 25° C. under N₂ for 16 hrs. The reaction mixture was diluted with EtOAc (15.0 mL), washed with water (15.0 mL), 1N HCl (10 mL×2), brine (20.0 mL), dried over anhydrous sodium sulfate, filtered, and the filtrate was concentrated to afford a residue. The crude product was purified by flash chromatography on silica gel (95% of EtOAc in PE) to afford methyl 4-((4-(2-(2-aminopyridin-3-yl)-5-phenyl-3H-imidazo[4,5-b]pyridin-3-yl)-3-fluorophenyl)carbamoyl)cyclohexane-1-carboxylate E4 (110 mg, 77.2% yield) as a yellow solid. ¹H NMR (400 MHz, CDCl₃) δ=8.16-8.05 (m, 2H), 8.03-7.96 (m, 2H), 7.84-7.75 (m, 2H), 7.44-7.31 (m, 6H), 7.23 (dd, J=1.6, 7.6 Hz, 1H), 6.60 (s, 2H), 6.43 (dd, J=4.8, 7.6 Hz, 1H), 3.73-3.69 (m, 3H), 2.65 (t, J=4.8 Hz, 1H), 2.41 (s, 1H), 2.30-2.15 (m, 2H), 1.95-1.63 (m, 6H).

Step 5. To a stirred solution of methyl 4-((4-(2-(2-aminopyridin-3-yl)-5-phenyl-3H-imidazo[4,5-b]pyridin-3-yl)-3-fluorophenyl)carbamoyl)cyclohexane-1-carboxylate E4 (100 mg, 0.180 mmol) in THF (1.00 mL) and water (1.00 mL), LiOH·H₂O (14.0 mg, 0.350 mmol) was added and the reaction mixture was stirred at 25° C. under N₂ for 5 hrs. The reaction mixture was diluted with water (5.00 mL), washed with EtOAc (5.00 mL) and the water phase was adjusted to pH ~5 with 1N HCl. The mixture was filtered and the filter cake residue collected. The crude product was purified by prep-HPLC (column: Welch Xtimate C18 150×30 mm×5 μm, method: water (FA)-ACN, begin B: 31, end B: 61) to afford 4-((4-(2-(2-aminopyridin-3-yl)-5-phenyl-3H-imidazo[4,5-b]pyridin-3-yl)-3-fluorophenyl)carbamoyl)cyclohexane-1-carboxylic acid I-5 (25.0 mg, 25.3% yield) as a white solid. ¹H NMR (400 MHz, DMSO-d₆) δ=12.17 (s, 1H), 10.31 (s, 1H), 8.28 (d, J=8.4 Hz, 1H), 8.09-7.95 (m, 4H), 7.85 (dd, J=2.0, 12.8 Hz, 1H), 7.70-7.57 (m, 1H), 7.56-7.33 (m, 4H), 7.25 (dd, J=1.6, 7.6 Hz, 1H), 7.15 (s, 2H), 6.46 (dd, J=4.8, 7.6 Hz, 1H), 2.57-2.52 (m, 1H), 2.47-2.17 (m, 1H), 2.10-1.95 (m, 2H), 1.92-1.20 (m, 6H). ¹⁹F NMR (376.5 MHz, DMSO-d₆) δ=−120.099. HPLC Rₜ=3.799 min in 8 min chromatography, purity 100%. LCMS Rₜ=2.055 min in 4 min chromatography, purity 100%, MS ESI calcd. for 550.21 [M+H]⁺ 551.21, found 551.1.

### Example 6. Synthesis of (1r,4r)-((4-(2-(2-aminopyridin-3-yl)-5-phenyl-3H-imidazo[4,5-b]pyridin-3-yl)-2-fluorophenyl)carbamoyl)cyclohexane-1-carboxylic acid (Compound I-6)

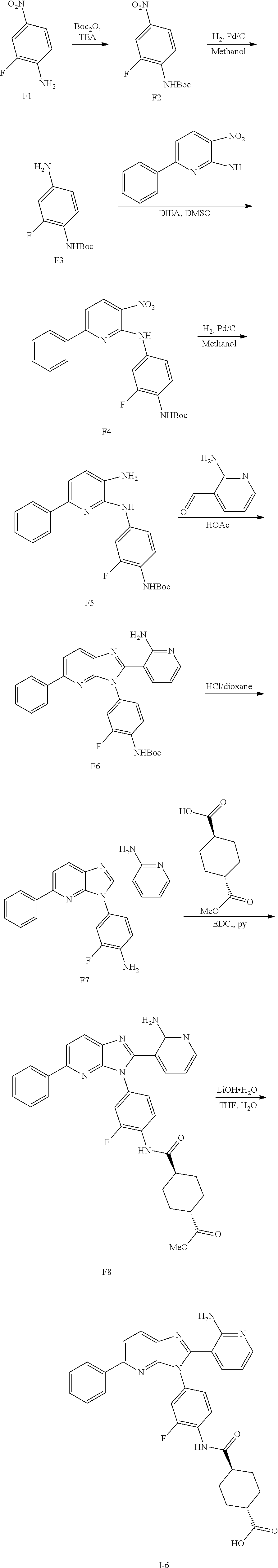

Step 1. To a solution of 2-fluoro-4-nitro-aniline F1 (5.00 g, 32.0 mmol) in DCM (50.0 mL), TEA (9.72 g, 96.1 mmol) and Boc₂0 (10.5 g, 48.0 mmol) were added at 25° C. and the mixture was stirred at 50° C. for 2 hrs. The mixture was diluted with DCM (100 mL) and washed with water (30 mL×2). The organic layer was dried over Na₂SO₄, filtered, and concentrated. The crude product was purified by column chromatography on silica gel (EtOAc in PE, 0-5%) to afford tert-butyl N-(2-fluoro-4-nitro-phenyl)carbamate F2 (5.60 g, 68.2% yield) as a yellow oil. ¹H NMR (DMSO-d₆, 400 MHz) δ_{H}=9.73 (s, 1H), 8.15-8.03 (m, 3H), 1.48 (s, 9H).

Step 2. To a solution of tert-butyl N-(2-fluoro-4-nitro-phenyl)carbamate F2 (5.60 g, 21.9 mmol) in methanol (20.0 mL), wet Pd/C (2.00 g, 10% purity) was added under an Ar atmosphere. The suspension was degassed and purged with H₂ (×3), and the reaction mixture was stirred under H₂ (15 psi) at 25° C. for 10 hrs. The reaction mixture was filtered via a celite pad, and the filter cake was washed with DCM/MeOH (10/1 (500 mL)). The filtrate was concentrated to afford tert-butyl N-(4-amino-2-fluoro-phenyl)carbamate F3 (3.80 g, 76.9% yield) as a yellow oil, which was used in the next step directly. ¹H NMR (DMSO-d₆, 400 MHz) δ_{H}=8.28 (s, 1H), 6.95 (s, 1H), 6.37-6.25 (m, 2H), 5.22 (s, 2H), 1.48-1.32 (s, 9H).

Step 3. To a solution of tert-butyl (4-amino-2-fluorophenyl)carbamate F3 (2.30 g, 9.80 mmol) in DMSO (20.0 mL), 2-chloro-3-nitro-6-phenylpyridine (2.66 g, 11.8 mmol) and DIEA (3.80 g, 29.4 mmol) were added at 25° C. and the mixture was stirred at 105° C. for 14 hrs. The reaction mixture was diluted with EtOAc (30 mL), washed with brine (10 mL×3), dried over anhydrous Na₂SO₄, filtered and concentrated. The residue was purified by flash silica gel chromatography (ISCO®; 40 g, SepaFlash® Silica Flash Column, eluent of 0-10% EtOAc in PE) to afford tert-butyl (2-fluoro-4-((3-nitro-6-phenylpyridin-2-yl)amino)phenyl)carbamate F4 (1.50 g, 36.1% yield) as a red solid. ¹H NMR (DMSO-d₆, 400 MHz) δ_{H}=10.11 (s, 1H), 8.93 (s, 1H), 8.62 (d, J=8.8 Hz, 1H), 8.09 (dd, J=2.8, 6.4 Hz, 2H), 7.76 (dd, J=2.0, 13.2 Hz, 1H), 7.62 (d, J=8.8 Hz, 1H), 7.59-7.52 (m, 4H), 7.50-7.45 (m, 1H), 1.47 (s, 9H)

Step 4. To a solution of tert-butyl (2-fluoro-4-((3-nitro-6-phenylpyridin-2-yl)amino)phenyl)carbamate F4 (480 mg, 1.13 mmol) in methanol (4.00 mL) and THF (4.00 mL), wet Pd/C (120 mg, 10%) was added under Ar atmosphere. The suspension was degassed and purged with H₂ (×3), and the reaction mixture was stirred under H₂ (15 psi) at 25° C. for 12 hrs. The reaction mixture was filtered and the filter cake was washed with MeOH/DCM (1/1, 500 mL). The resulting suspension was concentrated directly to afford tert-butyl (4-((3-amino-6-phenylpyridin-2-yl)amino)-2-fluorophenyl)carbamate F5 (470 mg, crude) as a yellow solid. LCMS Rₜ=1.480 min in 2.5 min chromatography, purity 55.6%, MS ESI calcd. for 394.18 [M+H]+ 395.18, found 395.0.

Step 5. A solution of 2-aminopyridine-3-carbaldehyde (161 mg, 1.32 mmol) and tert-butyl (4-((3-amino-6-phenylpyridin-2-yl)amino)-2-fluorophenyl)carbamate F5 (400 mg, 1.01 mmol) in acetic acid (3.0 mL) was stirred at 100° C. for 1 hr. The mixture was concentrated directly and the residue was purified by flash silica gel chromatography (ISCO®; 20 g, SepaFlash® Silica Flash Column, eluent with 0-50% of EtOAc in PE) to afford tert-butyl (4-(2-(2-aminopyridin-3-yl)-5-phenyl-3H-imidazo[4,5-b]pyridin-3-yl)-2-fluorophenyl)carbamate F6 (115 mg, 18.7% yield) as a yellow solid. ¹H NMR (DMSO-d₆, 400 MHz) δ=9.24 (s, 1H), 8.27 (d, J=8.4 Hz, 1H), 8.06-7.97 (m, 4H), 7.80 (t, J=8.4 Hz, 1H), 7.53-7.45 (m, 3H), 7.43-7.37 (m, 1H), 7.30 (dd, J1=1.6, 7.6 Hz, 1H), 7.25 (dd, J1=1.6, 8.8 Hz, 1H), 6.91 (s, 2H), 6.48 (dd, J=4.8, 7.6 Hz, 1H), 1.48 (s, 9H).

Step 6. To a solution of tert-butyl (4-(2-(2-aminopyridin-3-yl)-5-phenyl-3H-imidazo[4,5-b]pyridin-3-yl)-2-fluorophenyl)carbamate F6 (115 mg, 0.231 mmol) in DCM (1.00 mL), HCl/dioxane (1.00 mL, 4 M) was added and the mixture was stirred at 20° C. for 1 hr. The mixture was concentrated directly to afford 3-(3-(4-amino-3-fluorophenyl)-5-phenyl-3H-imidazo[4,5-b]pyridin-2-yl)pyridin-2-amine hydrochloride F7 (100 mg, crude) as a yellow solid. ¹H NMR (DMSO-d₆, 400 MHz) δ=8.74-8.40 (m, 2H), 8.33 (dd, J=2.8, 8.4 Hz, 1H), 8.13 (d, J=5.6 Hz, 1H), 8.05 (d, J=3.2 Hz, 4H), 7.92 (d, =6.8 Hz, 1H), 7.53-7.41 (m, 4H), 7.35 (d, J=11.6 Hz, 1H), 7.07 (d, J=8.0 Hz, 1H), 6.97-6.89 (m, 2H).

Step 7. To a solution of 3-(3-(4-amino-3-fluorophenyl)-5-phenyl-3H-imidazo[4,5-b]pyridin-2-yl)pyridin-2-amine F7 (200 mg, 0.510 mmol) in pyridine (3.00 mL), (1r,4r)-4-(methoxycarbonyl)cyclohexane-1-carboxylic acid (94.0 mg, 0.505 mmol) and EDCI (290 mg, 1.51 mmol) were added at 25° C., and the mixture was stirred at 80° C. for 2 hrs. The mixture was diluted with EtOAc (10 mL), washed with brine (5 mL×3). The combined organic phase was dried over anhydrous Na₂SO₄, filtered and concentrated. The residue was purified by Combi-Flash column (EtOAc in PE, 0%-60%) to afford methyl (1r,4r)-4-((4-(2-(2-aminopyridin-3-yl)-5-phenyl-3H-imidazo[4,5-b]pyridin-3-yl)-2-fluorophenyl)carbamoyl)cyclohexane-1-carboxylate F8 (94.0 mg, 26.6% yield) as a yellow solid. ¹H NMR (400 MHz, DMSO-d₆) δ_{H}=9.84 (s, 1H), 8.27 (d, J=8.4 Hz, 1H), 8.10-795 (m, 5H), 7.54 (dd, J=2.0, 11.2 Hz, 1H), 7.51-7.44 (m, 2H), 7.43-7.37 (m, 1H), 7.34-7.24 (m, 2H), 6.90 (s, 2H), 6.49 (dd, J=4.8, 7.6 Hz, 1H), 3.60 (s, 3H), 2.02-1.95 (m, 2H), 1.91 (d, J=10.8 Hz, 2H), 1.55-1.32 (m, 6H).

Step 8. To a solution of methyl (1r,4r)-4-((4-(2-(2-aminopyridin-3-yl)-5-phenyl-3H-imidazo[4,5-b]pyridin-3-yl)-2-fluorophenyl)carbamoyl)cyclohexane-1-carboxylate F8 (95.0 mg, 0.170 mmol) in THF (2.00 mL) and water (2.00 mL), LiOH·H₂O (21.0 mg, 0.510 mmol) was added and the mixture was stirred at 25° C. for 2 hrs. The reaction mixture was acidified to pH ~4 with aqueous HCl (2 M) and then purified by prep-HPLC (column: Xtimate C18 150*40 mm*10 μm; mobile phase: [water (HCl)-ACN]; B %: 10%-50%, 25 min) to afford (1r,4r)-4-((4-(2-(2-aminopyridin-3-yl)-5-phenyl-3H-imidazo[4,5-b]pyridin-3-yl)-2-fluorophenyl)carbamoyl)cyclohexane-1-carboxylic acid I-6 (52.1 mg, 56.2% yield) as a yellow solid. ¹H NMR (400 MHz, MeOD) δ_{H}=8.29 (d, J=8.4 Hz, 1H), 8.25-8.16 (m, 1H), 8.10-7.97 (m, 4H), 7.92 (dd, J=1.2, 7.6 Hz, 1H), 7.53 (dd, J=2.0, 10.8 Hz, 1H), 7.48-7.36 (m, 3H), 7.32 (d, J1=8.4 Hz, 1H), 6.91 (t, J=7.2 Hz, 1H), 2.62-2.50 (m, 1H), 2.38-2.28 (m, 1H), 2.17-1.98 (m, 4H), 1.70-1.47 (m, 4H).

### Example 7. Synthesis of 3-((5-(2-(2-aminopyridin-3-yl)-5-phenyl-3H-imidazo[4,5-b]pyridin-3-yl)pyridin-2-yl)carbamoyl)bicyclo[1.1.1]pentane-1-carboxylic acid (Compound I-7)

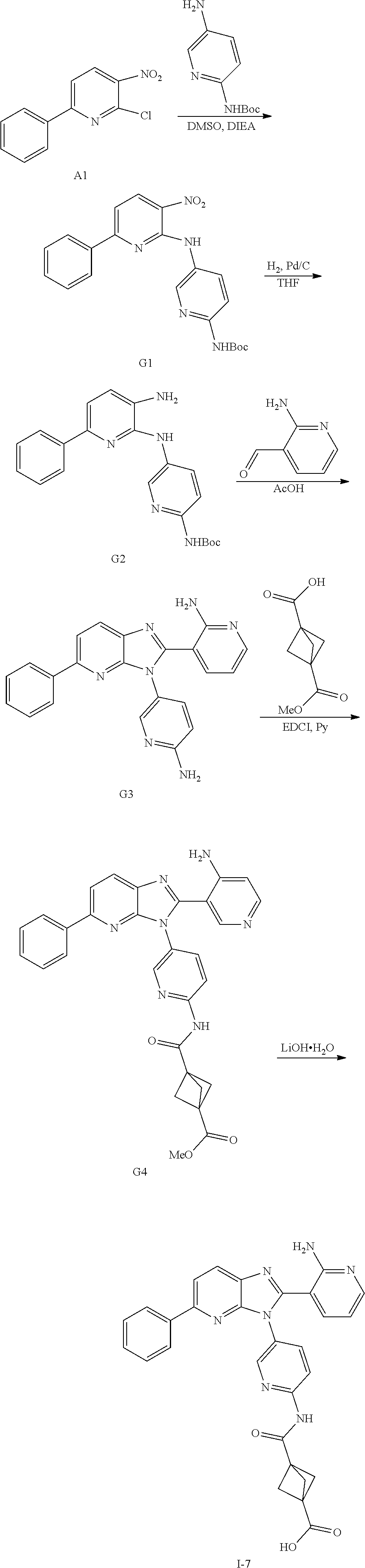

Step 1. To a solution of 2-chloro-3-nitro-6-phenylpyridine A1 (1.70 g, 7.25 mmol) in DMSO (15.0 mL), tert-butyl (5-aminopyridin-2-yl)carbamate (1.82 g, 8.69 mmol) and DIEA (2.81 g, 21.7 mmol) were added at 25° C. and the mixture was stirred at 80° C. for 14 hrs. EtOAc (20 mL) was added and the resulting suspension was washed with saturated aqueous LiCl (20 mL×3), dried over anhydrous Na₂SO₄, filtered and concentrated. The mixture was then triturated with isopropyl ether (20 mL) for 10 mins and filtered. The filter cake was concentrated to give tert-butyl (5-((3-nitro-6-phenylpyridin-2-yl)amino)pyridin-2-yl)carbamate G1 (3.80 g, crude) as a red solid. ¹H NMR (CDCl₃, 400 MHz) δ_{H}=10.18 (s, 1H), 8.65-8.55 (m, 2H), 8.18 (d, J=9.2 Hz, 1H), 8.09-7.98 (m, 3H), 7.66-7.58 (m, 1H), 7.52 (s, 3H), 7.36 (d, J=8.4 Hz, 1H), 1.57 (s, 9H).

Step 2. To a mixture of tert-butyl (5-((3-nitro-6-phenylpyridin-2-yl)amino)pyridin-2-yl)carbamate G1 (2.50 g, 9.68 mmol) in THF (20.0 mL), wet Pd/C (500 mg, 10.0% purity) was added under Ar atmosphere. The suspension was degassed and purged with H₂ (×3), and the mixture was stirred under H₂ (15 psi) at 25° C. for 14 hrs. The mixture was filtered via a celite pad, and the pad was subsequently washed with DCM/MeOH (1/1, 150 mL×5). The filtrate was concentrated to give tert-butyl (5-((3-amino-6-phenylpyridin-2-yl)amino)pyridin-2-yl)carbamate G2 (2.00 g, 79.4% yield) as a yellow solid, which was used in the next step without further purification. ¹H NMR (DMSO-d₆, 400 MHz) δ_{H}=9.53 (s, 1H), 8.74 (d, J=2.4 Hz, 1H), 8.10 (dd, J=2.8, 9.2 Hz, 1H), 7.98 (s, 1H), 7.90 (dd, J=1.6, 8.8 Hz, 2H), 7.73 (d, J=9.2 Hz, 1H), 7.40 (t, J=7.6 Hz, 2H), 7.29-7.22 (m, 2H), 6.98 (d, J=7.6 Hz, 1H), 5.28 (s, 2H), 1.48 (s, 9H).

Step 3. To a solution of tert-butyl (5-((3-amino-6-phenylpyridin-2-yl)amino)pyridin-2-yl)carbamate G2 (2.00 g, 5.30 mmol) in acetic acid (20.0 mL), 2-aminopyridine-3-carbaldehyde (777 mg, 6.36 mmol) was added at 25° C., and the mixture was stirred at 100° C. for 24 hrs. The reaction mixture was concentrated and the resulting residue was purified by flash column (EtOAc in PE, 0%-100%) to give 3-(3-(6-aminopyridin-3-yl)-5-phenyl-3H-imidazo[4,5-b]pyridin-2-yl)pyridin-2-amine G3 (320 mg, 15.9% yield) as a yellow solid. LCMS Rₜ=0.938 min in 2.5 min chromatography, purity 60.3%, MS ESI calcd. for 379.15, [M+H]⁺ 380.15, found 380.1.

Step 4. To a mixture of 3-(3-(6-aminopyridin-3-yl)-5-phenyl-3H-imidazo[4,5-b]pyridin-2-yl)pyridin-2-amine G3 (100 mg, 0.260 mmol) in pyridine (3.00 mL), 3-(methoxycarbonyl)bicyclo[1.11.]pentane-1-carboxylic acid (54.0 mg, 0.310 mmol) and EDCI (150 mg, 0.790 mmol) were added and the mixture was stirred at 80° C. for 2 hrs. Water (10 mL) was added, and the resulting mixture was extracted with EtOAc (15 mL×3) and washed with brine (20 mL×2). The combined organic phase was dried over anhydrous sodium sulfate, filtered, and the filtrate was concentrated. The residue was purified by flash chromatography on silica gel (75% of EtOAc in PE) to afford methyl 3-((5-(2-(2-aminopyridin-3-yl)-5-phenyl-3H-imidazo[4,5-b]pyridin-3-yl)pyridin-2-yl)carbamoyl)bicyclo[1.1.1]pentane-1-carboxylate G4 (70.0 mg, 49.9% yield) as a yellow oil. LCMS Rₜ=1.417 min in 2.0 min chromatography, purity 83.1%, MS ESI calcd. for 683.25 [M+H]⁺ 684.25, found 684.3.

Step 5. To a solution of methyl 3-((5-(2-(2-aminopyridin-3-yl)-5-phenyl-3H-imidazo[4,5-b]pyridin-3-yl)pyridin-2-yl)carbamoyl)bicyclo[1.1.1]pentane-1-carboxylate G4 (70.0 mg, 0.140 mmol) in THF (2.00 mL) and water (2.00 mL), LiOH·H₂O (16.0 mg, 0.420 mmol) was added at 25° C., and the mixture was stirred for 2 hrs. Formic acid (1 mL) was added to the mixture and the residue was purified by prep-HPLC (condition: water (FA)-ACN; Column: Xtimate C18 150*40 mm*10 μm; B %: 8%-48%; Gradient Time, 25 min) to afford 3-((5-(2-(2-aminopyridin-3-yl)-5-phenyl-3H-imidazo[4,5-b]pyridin-3-yl)pyridin-2-yl)carbamoyl)bicyclo[1.1.1]pentane-1-carboxylic acid I-7 (17.2 mg, 29.9% yield) as a white solid. ¹H NMR (400 MHz, DMSO-d₆) δ=10.63 (s, 1H), 8.50 (dd, J=1.6, 5.2 Hz, 1H), 8.21-8.15 (m, 2H), 8.07 (d, J=7.6 Hz, 2H), 7.95 (d, J=8.4 Hz, 1H), 7.69-7.61 (m, 2H), 7.52-7.46 (m, 2H), 7.44-7.38 (m, 1H), 7.29-7.24 (m, 1H), 6.52 (d, J=8.8 Hz, 1H), 6.28 (s, 2H), 2.10 (s, 6H). HPLC Rₜ=1.830 min in 8 min chromatography, purity 98.5%. LCMS Rₜ=1.290 min in 4 min chromatography, purity 99.7%, MS ESI calcd. for 517.19 [M+H]⁺ 518.19, found 518.2.

### Example 8. Synthesis of 3-((5-(2-(2-aminopyridin-3-yl)-5-phenyl-3H-imidazo[4,5-b]pyridin-3-yl)pyridin-2-yl)carbamoyl)cyclopentane-1-carboxylic acid (Compound I-8)

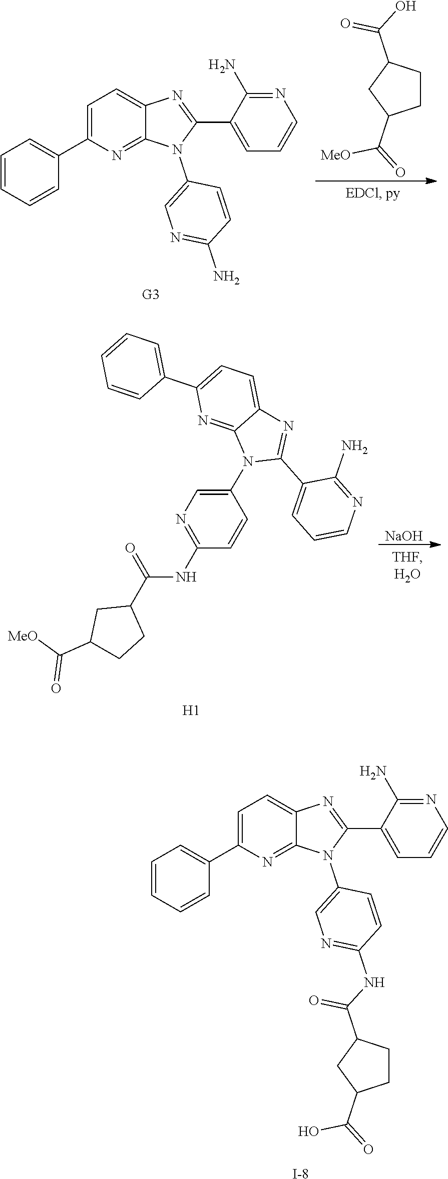

Step 1. To a mixture of 3-(3-(6-aminopyridin-3-yl)-5-phenyl-3H-imidazo[4,5-b]pyridin-2-yl)pyridin-2-amine G3 (150 mg, 0.395 mmol) and 3-(methoxycarbonyl)cyclopentane-1-carboxylic acid (68.0 mg, 0.395 mmol) in pyridine (3.00 mL), EDCI (341 mg, 1.780 mmol) was added and the mixture was stirred at 80° C. for 2 hrs. Water (10 mL) was added, and the resulting mixture was extracted with EtOAc (15 mL×3) and washed with brine (20 mL×2). The combined organic phase was dried over anhydrous sodium sulfate, filtered, and the filtrate was concentrated to afford methyl 3-((5-(2-(2-aminopyridin-3-yl)-5-phenyl-3H-imidazo[4,5-b]pyridin-3-yl)pyridin-2-yl)carbamoyl)cyclopentane-1-carboxylate H1 (140 mg, 66.4% yield) as a yellow oil, which was used in the next step without further purification LCMS Rₜ=0.576 min in 1.0 min chromatography, purity 39.8%, MS ESI calcd. for 533.22 [M+H]⁺ 534.22, found 534.2.

Step 2. To a solution of methyl 3-((5-(2-(2-aminopyridin-3-yl)-5-phenyl-3H-imidazo[4,5-b]pyridin-3-yl)pyridin-2-yl)carbamoyl)cyclopentane-1-carboxylate H1 (140 mg, 0.262 mmol) in THF (2.00 mL), NaOH (10.0 mg, 0.262 mmol) and water (1.0 mL) were added at 25° C., and the mixture was stirred for 12 hrs. The reaction mixture was filtered, and the filtrate was concentrated to dryness. The resulting residue was purified by prep-HPLC (condition: water (NH₄HCO. )-ACN; Column Xtimate C18 150*40 mm*10 μm; B %: 2%-42%; Gradient Time, 25 min) to afford 3-((5-(2-(2-aminopyridin-3-yl)-5-phenyl-3H-imidazo[4,5-b]pyridin-3-yl)pyridin-2-yl)carbamoyl)cyclopentane-1-carboxylic acid I-8 (6.20 mg, 4.48% yield) as a white solid. ¹H NMR (400 MHz, DMSO-d₆) δ=10.81 (d, J=2.4 Hz, 1H), 8.43 (d, J=2.0 Hz, 1H), 8.32-8.25 (m, 2H), 8.08-8.01 (m, 4H), 7.97 (dd, J=2.8, 8.8 Hz, 1H), 7.51-7.46 (m, 2H), 7.43-7.35 (m, 2H), 6.86 (s, 2H), 6.52 (dd, J=4.8, 7.6 Hz, 1H), 3.05-2.98 (m, 1H), 2.78-2.71 (m, 1H), 2.21-2.13 (m, 1H), 2.02-1.80 (m, 5H). HPLC Rₜ=3.913 min in 8 min chromatography, purity 98.6%. LCMS Rₜ=0.768 min in 2 min chromatography, purity 96.2%, MS ESI calcd. for 519.20 [M+H]⁺ 520.20, found 520.1.

### Example 9. Synthesis of 4-((5-(2-(2-aminopyridin-3-yl)-5-phenyl-3H-imidazo[4,5-b]pyridin-3-yl)pyridin-2-yl)carbamoyl)bicyclo[2.1.1]hexane-1-carboxylic acid (Compound I-9)

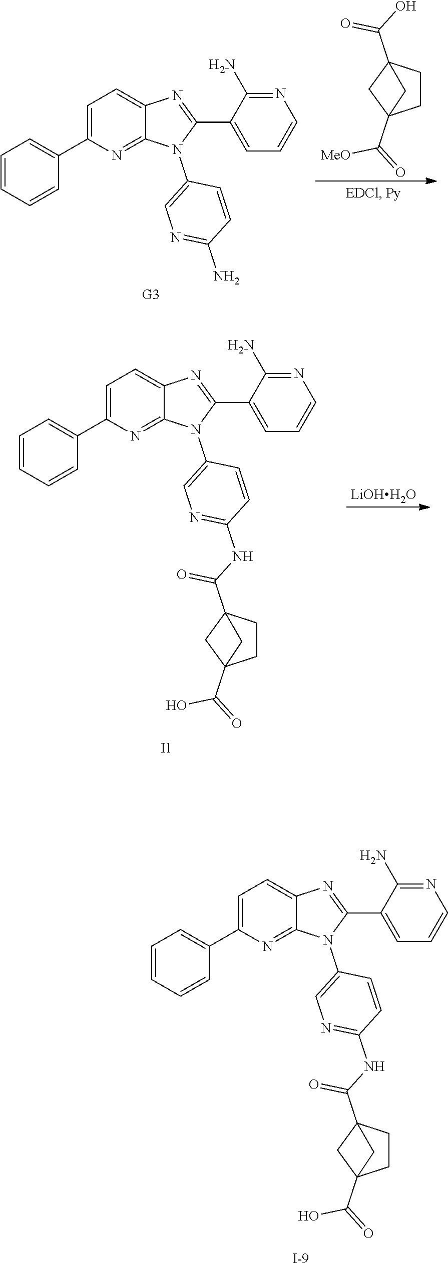

Step 1. To a mixture of 3-(3-(6-aminopyridin-3-yl)-5-phenyl-3H-imidazo[4,5-b]pyridin-2-yl)pyridin-2-amine G3 (150 mg, 0.390 mmol) in pyridine (3.00 mL), 4-(methoxycarbonyl)bicyclo[2.1.1]hexane-1H-carboxylic acid (80.0 mg, 0.430 mmol) and EDCI (230 mg, 1.19 mmol) were added and the mixture was stirred at 80° C. for 2 hrs. Water (10 mL) was added, and the resulting mixture was extracted with EtOAc (15 mL 3) and washed with brine (20 mL×2). The combined organic phase was dried over anhydrous sodium sulfate, filtered and the filtrate was concentrated. The residue was purified by flash chromatography on silica gel (70% of EtOAc in PE) to afford methyl 4-((5-(2-(2-aminopyridin-3-yl)-5-phenyl-3H-imidazo[4,5-b]pyridin-3-yl)pyridin-2-yl)carbamoyl)bicyclo[2.1.1]hexane-1-carboxylate 11 (80.0 mg, 37.1% yield) as a yellow oil. LCMS Rₜ=0.644 min in 1.0 min chromatography, purity 56.4%, MS ESI calcd. for 545.22 [M+H]⁺ 546.22, found 546.3.

Step 2. To a solution of methyl 4-((5-(2-(2-aminopyridin-3-yl)-5-phenyl-3H-imidazo[4,5-b]pyridin-3-yl)pyridin-2-yl)carbamoyl)bicyclo[2.1.1]hexane-1-carboxylate 11 (80.0 mg, 0.140 mmol) in THF (2.00 mL) and water (2.00 mL), LiOH·H₂O (17.0 mg, 0.420 mmol) was added at 25° C. and the mixture was stirred for 2 hrs. Formic acid (1 mL) was added to the mixture and the residue was purified by prep-HPLC (condition: water (HCl)-ACN; Column: Xtimate C18 150*40 mm*10 μm; B %: 6%-46%; Gradient Time, 20 min) and prep-HPLC (condition: water (FA)-ACN; Column: Xtimate C18 150*40 mm*10 μm; B %: 4%-44%; Gradient Time, 30 min) to afford 4-((5-(2-(2-aminopyridin-3-yl)-5-phenyl-3H-imidazo[4,5-b]pyridin-3-yl)pyridin-2-yl)carbamoyl)bicyclo[2.1.1]hexane-1-carboxylic acid I-9 (8.00 mg, 13.7% yield) as a white solid. ¹H NMR (400 MHz, DMSO-d₆) δ=10.42 (s, 1H), 8.46 (d, J=2.4 Hz, 1H), 8.28 (dd, J=8.4, 14.8 Hz, 2H), 8.08-8.01 (m, 4H), 7.99 (dd, J=2.8, 8.8 Hz, 1H), 7.51-7.45 (m, 2H), 7.44-7.36 (m, 2H), 6.85 (s, 2H), 6.53 (dd, J=4.8, 6.8 Hz, 1H), 2.04 (s, 4H), 1.95-1.87 (m, 2H), 1.78 (d, J=2.4 Hz, 2H). HPLC Rₜ=2.493 min in 8 min chromatography, purity 99.3%. LCMS Rₜ=1.646 min in 4 min chromatography, purity 99.2%, MS ESI calcd. for 531.20 [M+H]⁺ 532.20, found 532.2.

### Example 10. Synthesis of (1r,4r)-4-((5-(2-(2-aminopyridin-3-yl)-5-phenyl-3H-imidazo[4,5-b]pyridin-3-yl)pyridin-2-yl)carbamoyl)cyclohexane-1-carboxylic acid (Compound I-10)

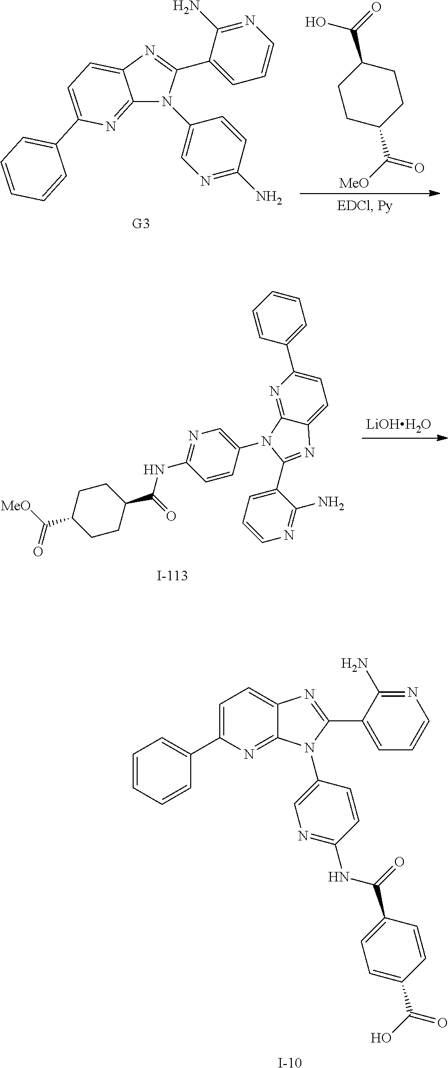

Step 1. To a mixture of 3-(3-(6-aminopyridin-3-yl)-5-phenyl-3H-imidazo[4,5-b]pyridin-2-yl)pyridin-2-amine G3 (130 mg, 0.340 mmol) and (1r,4r)-4-(methoxycarbonyl)cyclohexane-1-carboxylic acid (96.0 mg, 0.510 mmol) in pyridine (3.00 mL), EDCI (197 mg, 1.03 mmol) was added and the mixture was stirred at 80° C. for 2 hrs. Water (10 mL) was added, and the resulting mixture was extracted with EtOAc (15 mL 3) and washed with brine (20 mL×2). The combined organic phase was dried over anhydrous sodium sulfate, filtered and the filtrate was concentrated. The residue was purified by flash chromatography on silica gel (70% of EtOAc in PE) to afford trans-methyl 4-[[5-[2-(2-amino-3-pyridyl)-5-phenyl-imidazo[4,5-b]pyridin-3-yl]-2-pyridyl]carbamoyl]cyclohexanecarboxylate 1-113 (80.0 mg, 42.6% yield) as a yellow oil. LCMS Rₜ=1.350 min in 2 min chromatography, purity 47.0%, MS ESI calcd. for 547.23 [M+H]⁺ 548.23, found 548.2.

Step 2. To a solution of trans-methyl 4-[[5-[2-(2-amino-3-pyridyl)-5-phenyl-imidazo[4,5-b]pyridin-3-yl]-2-pyridyl]carbamoyl]cyclohexanecarboxylate I-113 (80.0 mg, 0.140 mmol) in THF (2.00 mL) and water (2.00 mL), LiOH·H₂O (18.0 mg, 0.430 mmol) was added at 25° C. and the mixture was stirred for 2 hrs. Formic acid (1 mL) was subsequently added to the mixture, and the residue was purified by prep-HPLC (condition: water (FA)-ACN; Column. Xtimate C18 150*40 mm*10 μm; B %: 8%-48%; Gradient Time, 25 min) to afford (1r,4r)-4-((5-(2-(2-aminopyridin-3-yl)-5-phenyl-3H-imidazo[4,5-b]pyridin-3-yl)pyridin-2-yl)carbamoyl)cyclo hexane-1-carboxylic acid 1-10 (17.6 mg, 22.5% yield) as a white solid. ¹H NMR (400 MHz, DMSO-d₆) δ=12.09 (s, 1H), 10.65 (s, 1H), 8.42 (d, J=2.8 Hz, 1H), 8.27 (dd, J=8.4, 12.0 Hz, 2H), 8.09-7.99 (m, 4H), 7.96 (dd, J=2.8, 8.8 Hz, 1H), 7.48 (d, J=7.2 Hz, 2H), 7.44-7.40 (m, 1H), 7.39-7.34 (m, 1H), 6.84 (s, 2H), 6.54-6.49 (m, 1H), 2.61-2.56 (m, 1H), 2.27-2.14 (m, 1H), 2.04-1.85 (m, 4H), 1.55-1.41 (m, 2H), 1.40-1.28 (m, 2H). HPLC Rₜ=2.579 min in 8 min chromatography, purity 99.6%. LCMS Rₜ=1.698 min in 4 min chromatography, purity 99.7%, MS ESI calcd. for 533.22 [M+H]⁺ 534.22, found 534.2.

### Example 11. Synthesis of 3-((5-(2-(2-aminopyridin-3-yl)-5-phenyl-3H-imidazo[4,5-b]pyridin-3-yl)pyridin-2-yl)carbamoyl)cyclohexane-1-carboxylic acid (Compound I-11)

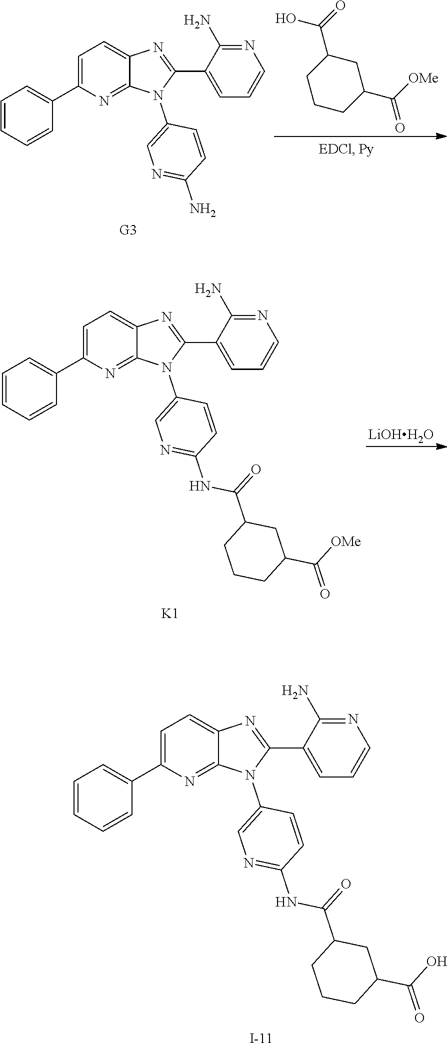

Step 1. To a mixture of 3-(3-(6-aminopyridin-3-yl)-5-phenyl-3H-imidazo[4,5-b]pyridin-2-yl)pyridin-2-amine G3 (150 mg, 0.390 mmol) and 4-(methoxycarbonyl)-2-methylbenzoic acid (74.0 mg, 0.390 mmol) in pyridine (3.00 mL), EDCI (227 mg, 1.19 mmol) was added and the mixture stirred at 80° C. for 2 hrs. Water (10 mL) was added, and the resulting mixture was extracted with EtOAc (15 mL×3) and washed with brine (20 mL×2). The combined organic phase was dried over anhydrous sodium sulfate, filtered and the filtrate was concentrated. The residue was purified by flash chromatography on silica gel (70% of EtOAc in PE) to afford methyl 3-[[5-[2-(2-amino-3-pyridyl)-5-phenyl-imidazo[4,5-b]pyridin-3-yl]-2-pyridyl]carbamoyl]cyclohexanecarboxylate K1 (70.0 mg, 32.3% yield) as a yellow oil. LCMS Rₜ=0.765 min in 1.5 min chromatography, purity 66.6%, MS ESI calcd. for 547.23 [M+H]⁺ 548.23, found 548.0.

Step 2. To a solution of methyl 3-[[5-[2-(2-amino-3-pyridyl)-5-phenyl-imidazo[4,5-b]pyridin-3-yl]-2-pyridyl]carbamoyl]cyclohexanecarboxylate K1 (70.0 mg, 0.120 mmol) in THF (2.00 mL) and water (2.00 mL), LiOH·H₂O (16.0 mg, 0.380 mmol) was added at 25° C., and the mixture stirred for 2 hrs. Formic acid (I mL) was added to the mixture, and the residue was purified by prep-HPLC (condition: water (FA)-ACN; Column. Xtimate C18 150*40 mm*10 μm: B %: 8%-48%: Gradient Time, 25 min) to afford 3-((5-(2-(2-aminopyridin-3-yl)-5-phenyl-3H-imidazo[4,5-b]pyridin-3-yl)pyridin-2-yl)carbamoyl)cyclohexane-1-carboxylic acid I-11 (9.80 mg, 13.4% yield) as a white solid. ¹H NMR (400 MHz, DMSO-d₆) δ=12.12 (s, 1H), 10.67 (s, 1H), 8.43 (d, J=2.8 Hz, 1H), 8.27 (dd, J=8.4, 13.2 Hz, 2H), 8.09-7.99 (m, 4H), 7.96 (dd, J=2.4, 9.2 Hz, 1H), 7.47 (d, J=7.2 Hz, 2H), 7.44-7.38 (m, 2H), 6.93 (s, 1H), 6.59-6.49 (m, 1H), 2.61-2.56 (m, 1H), 2.30-2.20 (m, 1H), 2.11-1.98 (m, 1H), 1.96-1.77 (m, 3H), 1.55-1.42 (m, 1H), 1.38-1.21 (m, 3H). HPLC Rₜ=2.939 min in 8 min chromatography, purity 93.0%. LCMS Rₜ=1.784 min in 4 min chromatography, purity 93.7%, MS ESI calcd. for 533.22 [M+H]⁺ 534.22, found 534.2.

### Example 12. Synthesis of 4-(((5-(2-(2-aminopyridin-3-yl)-5-phenyl-3H-imidazo[4,5-b]pyridin-3-yl)pyridin-2-yl)amino)methyl)cyclohexane-1-carboxylic acid (Compound I-12)

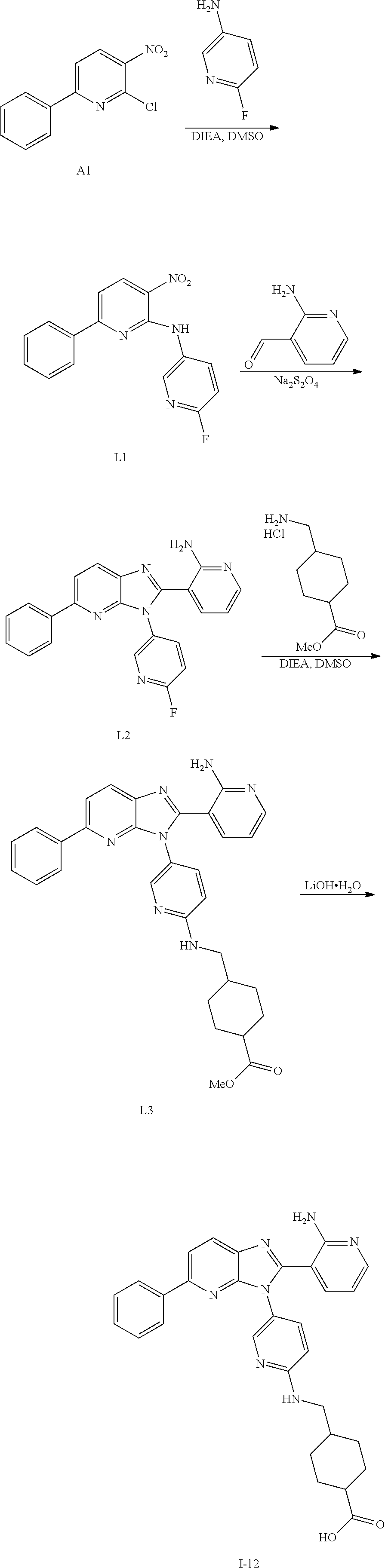

Step 1. To a stirred solution of 2-chloro-3-nitro-6-phenylpyridine A1 (5.00 g, 21.3 mmol) and 6-fluoropyridin-3-amine (4.17 g, 37.2 mmol) in DMSO (100 mL), DIEA (12.0 mL, 63.9 mmol) was added and the reaction was stirred at 90° C., under N₂, for 16 hrs. The reaction mixture was poured into MeOH (200 mL) and triturated to give a crude material. The crude material was filtered, and the filter cake was washed with MTBE (100 mL), dried in vacuum to give N-(6-fluoropyridin-3-yl)-3-nitro-6-phenylpyridin-2-amine L1 (5.00 g, 75.6% yield) as a yellow solid. ¹H NMR (DMSO-d₆, 400 MHz) δ_{H}=10.12 (s, 1H), 8.61 (d, J=8.4 Hz, 1H), 8.52 (s, 1H), 8.30-8.23 (m, 1H), 8.04-7.97 (m, 2H), 7.61 (d, J=8.8 Hz, 1H), 7.54-7.48 (m, 3H), 7.26 (dd, J=8.8, 3.2 Hz, 1H). ¹⁹F NMR (DMSO-<4s, 376.5 MHz) δ=−73.725.

Step 2. To a solution of N-(6-fluoropyridin-3-yl)-3-nitro-6-phenylpyridin-2-amine L1 (2.00 g, 6.45 mmol) in DMSO (13.0 mL) and MeOH (2.00 mL), 2-aminonicotinaldehyde (1.10 g, 9.02 mmol) and Na₂S₂O₄ (4.49 g, 25.8 mmol) were added and the reaction mixture was stirred at 100° C. for 16 hrs. Water (130 mL) was added to the mixture, and the whole extracted with EtOAc (60 mL×3). The organic layer was dried over anhydrous sodium sulfate, filtered and the filtrate was concentrated. The resulting residue was purified by flash chromatography on silica gel (0-100/o of EtOAc in PE) to give 3-(3-(6-fluoropyridin-3-yl)-5-phenyl-3H-imidazo[4,5-b]pyridin-2-yl)pyridin-2-amine L2 (1.40 g, 56.8% yield) as a red solid. ¹H NMR (CDCl₃, 400 MHz) δ=8.45 (d, J=2.4 Hz, 1H), 8.20-8.10 (m, 2H), 8.03 (d, J=7.6 Hz, 2H), 7.93-7.83 (m, 2H), 7.50-7.39 (m, 3H), 7.18-7.07 (m, 2H), 6.56 (s, 2H), 6.49 (dd, J=7.6, 4.8 Hz, 1H). ¹⁹F NMR (CDCl₃, 376.5 MHz) δ=−66.845.

Step 3. To a solution of methyl 4-(aminomethyl)cyclohexane-1-carboxylate;hydrochloride (163 mg, 0.785 mmol) in DMSO (2.00 mL), DIEA (0.100 mL, 0.588 mmol) and 3-(3-(6-fluoropyridin-3-yl)-5-phenyl-3H-imidazo[4,5-b]pyridin-2-yl)pyridin-2-amine L2 (75.0 mg, 0.196 mmol) were added and the reaction mixture was stirred at 80° C. for 16 hrs. Water (20 mL) was added, and the whole extracted with EtOAc (10 mL×3) The organic layer was dried over anhydrous sodium sulfate, filtered, and concentrated in vacuum. The crude was purified by flash column (0-80% of EtOAc in PE) to give methyl 4-[[[5-[2-(2-amino-3-pyridyl)-5-phenyl-imidazo[4,5-b]pyridin-3-yl]-2-pyridyl]amino]methyl]cyclohexanecarboxylate L3 (80.0 mg, 76.4% yield) as a red solid. ¹H NMR (400 MHz, DMSO-d₆) δ=8.24 (d, J=8.4 Hz, 1H), 8.06-8.00 (m, 4H), 7.96 (d, J=8.0 Hz, 1H), 7.51-7.44 (m, 3H), 7.43-7.35 (m, 2H), 7.00-6.88 (m, 3H), 6.59 (d, J=8.8 Hz, 1H), 6.50 (dd, J=7.6, 4.4 Hz, 1H), 3.58 (s, 3H), 3.13 (t, J=6.0 Hz, 2H), 1.96-1.81 (m, 4H), 1.60-1.44 (m, 1H), 1.39-1.12 (m, 3H), 1.10-0.90 (m, 2H).

Step 4. To a solution of methyl 4-(((5-(2-(2-aminopyridin-3-yl)-5-phenyl-3H-imidazo[4,5-b]pyridin-3-yl)pyridin-2-yl)amino)methyl)cyclohexane-1-carboxylate L3 (80.0 mg, 0.150 mmol) in THF (1.00 mL) and water (1.00 mL), LiOH·H₂O (13.0 mg, 0.300 mmol) was added and the reaction mixture was stirred at 25° C. for 16 hrs. The reaction mixture was used directly for purification by prep-HPLC (column. Welch Xtimate C18 150*30 mm*5 μm; mobile phase: water (HCl)-ACN; B %: 15%-45%, 30 min) to give 4-(((5-(2-(2-aminopyridin-3-yl)-5-phenyl-3H-imidazo[4,5-b]pyridin-3-yl)pyridin-2-yl)amino)methyl)cyclohexane-1-carboxylic acid I-12 (49.0 mg, 61.7% yield) as a white solid. ¹H NMR (400 MHz, DMSO-d₆) δ=8.34 (d, J=8.8 Hz, 1H), 8.20-8.01 (m, 6H), 7.99-7.91 (m, 1H), 7.80-7.69 (m, 1H), 7.53-7.39 (m, 3H), 6.93-6.88 (m, 1H), 3.19-3.16 (m, 2H), 2.22-2.12 (m, 1H), 1.99-1.81 (m, 4H), 1.63-1.47 (m, 1H), 1.40-1.22 (m, 2H), 1.09-0.94 (m, 2H). HPLC Rₜ=4.235 min in 8 min chromatography, purity 98.5%. LCMS Rₜ=2.336 min in 4 min chromatography, purity 100%, MS ESI calcd. For 519.24 [M+H]⁺ 520.24, found 520.3.

### Example 13. Synthesis of 4-((5-(2-(2-aminopyridin-3-yl)-5-phenyl-3H-imidazo[4,5-b]pyridin-3-yl)pyridin-2-yl)carbamoyl)bicyclo[2.1.1]hexane-1-carboxylic acid (Compound I-13)

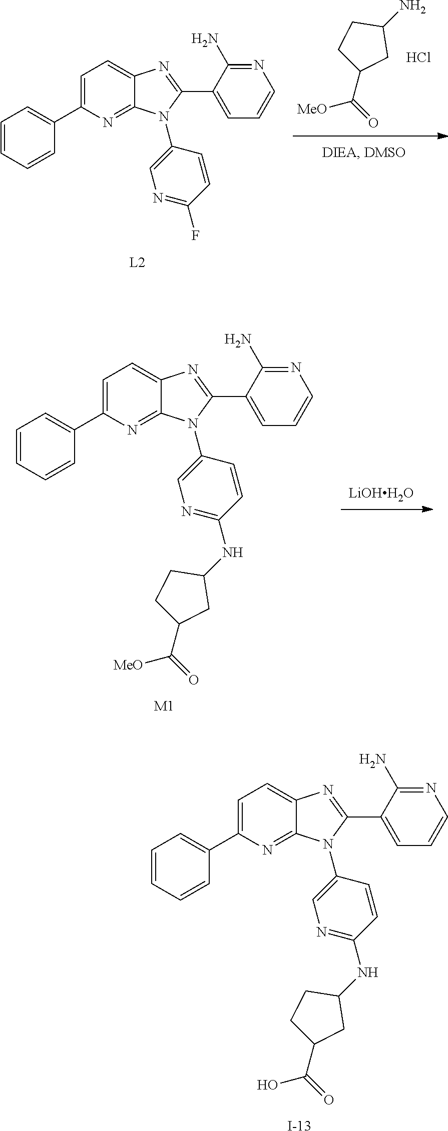

Step 1. To a solution of methyl 3-aminocyclopentane-1-carboxylate;hydrochloride (106 mg, 0.549 mmol) in DMSO (2.00 mL), DIEA (0.0960 mL, 0.549 mmol) and 3-(3-(6-fluoropyridin-3-yl)-5-phenyl-3H-imidazo[4,5-b]pyridin-2-yl)pyridin-2-amine L2 (70.0 mg, 0.183 mmol) were added and the mixture then stirred at 120° C. for 16 hrs. Water (20 mL) was added to the reaction mixture, and the whole extracted with EtOAc (10 mL×3). The organic layer was dried over anhydrous sodium sulfate and concentrated in vacuum. The crude was purified by flash column (0-80% of EtOAc in PE) to give methyl 3-((5-(2-(2-aminopyridin-3-yl)-5-phenyl-3H-imidazo[4,5-b]pyridin-3-yl)pyridin-2-yl)amino)cyclopentane-1-carboxylate M1 (80 mg, 84.1% yield) as a red solid. ¹H NMR (400 MHz, DMSO-d₆) δ=8.28-8.20 (m, 1H), 8.10-7.93 (m, 5H), 7.53-7.46 (m, 3H), 7.45-7.36 (m, 2H), 7.03 (t, J=5.2 Hz, 1H), 6.92 (s, 2H), 6.66-6.45 (m, 2H), 3.62 (s, 3H), 2.37-2.26 (m, 1H), 2.17-2.01 (m, 2H), 1.95-1.46 (m, 5H).

Step 2. To a solution of methyl 3-((5-(2-(2-aminopyridin-3-yl)-5-phenyl-3H-imidazo[4,5-b]pyridin-3-yl)pyridin-2-yl)amino)cyclopentane-1-carboxylate M1 (80.0 mg, 0.158 mmol) in THF (1.00 mL) and water (1.00 mL), LiOH·H₂O (13.0 mg, 0.316 mmol) was added and the reaction mixture was stirred at 25° C. for 16 hrs. The crude material was purified by prep-HPLC (column. Welch Xtimate C18 150*30 mm*5 μm, mobile phase: water (FA)-ACN; B %: 20%-30%, 30 min) to give 3-((5-(2-(2-aminopyridin-3-yl)-5-phenyl-3H-imidazo[4,5-b]pyridin-3-yl)pyridin-2-yl)amino)cyclopentane-1-carboxylic acid I-13 (11.0 mg, 14.0% yield) as a yellow solid. ¹H NMR (400 MHz, DMSO-d₆) δ=8.25 (d, J=8.4 Hz, 1H), 8.09-8.00 (m, 4H), 7.97 (d, J=8.4 Hz, 1H), 7.53-7.36 (m, 5H), 6.93 (s, 2H), 6.60-6.48 (m, 2H), 4.32-4.09 (m, 1H), 2.97-2.74 (m, 1H), 2.15-1.50 (m, 6H). HPLC Rₜ=4.083 min in 8 min chromatography, purity 98.0%. LCMS Rₜ=1.589 min in 4 min chromatography, purity 99.7%, MS ESI calcd. For 491.21 [M+H]⁺ 492.21, found 492.1.

### Example 14 and Example 15. Synthesis of (1r,4r)-4-((5-(2-(2-aminopyridin-3-yl)-5-phenyl-3H-imidazo[4,5-b]pyridin-3-yl)pyridin-2-yl)amino)cyclohexane-1-carboxylic acid (Compound I-14) and (1s,4s)-4((5-(2-(2-aminopyridin-3-yl)-5-phenyl-3H-imidazo[4,5-b]pyridin-3-yl)pyridin-2-yl)amino) cyclohexane-1-carboxylic acid (Compound I-15)

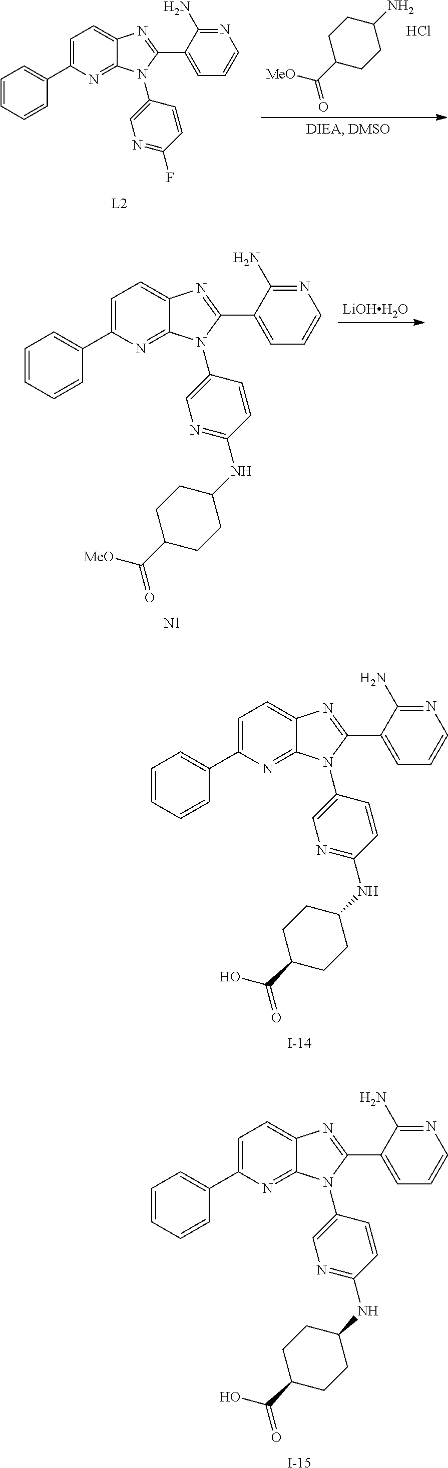

Step 1. To a solution of methyl 4-aminocyclohexane-1-carboxylate;hydrochloride (432 mg, 2.230 mmol) in DMSO (2.00 mL), DIEA (0.290 mL, 1.670 mmol) and 3-(3-(6-fluoropyridin-3-yl)-5-phenyl-3H-imidazo[4,5-b]pyridin-2-yl)pyridin-2-amine L2 (213 mg, 0.550 mmol) were added and the reaction was stirred at 80° C. for 16 hrs. Water (20 mL) was added to the reaction mixture and the whole extracted with EtOAc (30 mL×3). The organic layer was dried over anhydrous sodium sulfate, filtered, and concentrated in vacuum. The crude was purified by flash column (0-80% of EtOAc in PE) to give methyl 4-((5-(2-(2-aminopyridin-3-yl)-5-phenyl-3H-imidazo[4,5-b]pyridin-3-yl)pyridin-2-yl)amino)cyclohexane-1-carboxylate N1 (100 mg, 34.6% yield) as a red solid. ¹H NMR (400 MHz, DMSO-d₆) δ=8.23 (d, J=8.4 Hz, 1H), 8.08-7.91 (m, 4H), 7.50-7.36 (m, 4H), 6.93-6.80 (m, 2H), 6.65-6.46 (m, 2H), 3.64-3.58 (m, 3H), 2.08-2.01 (m, 1H), 2.00-1.81 (m, 3H), 1.77-1.16 (m, 6H).

Step 2. To a solution of methyl 4-((5-(2-(2-aminopyridin-3-yl)-5-phenyl-3H-imidazo[4,5-b]pyridin-3-yl)pyridin-2-yl)amino)cyclohexane-1-carboxylate N1 (100 mg, 0.205 mmol) in THF (1.00 mL) and water (1.00 mL), LiOH·H₂O (17.0 mg, 0.409 mmol) was added and the reaction mixture was stirred at 25° C. for 16 hrs. The reaction mixture was used directly for purification by prep-HPLC (column Welch Xtimate C18 150*40 mm*5 μm; mobile phase: water (HCl)-ACN; B %: 8%-38%, 60 min) to give (1s,4s)-4-((5-(2-(2-aminopyridin-3-yl)-5-phenyl-3H-imidazo[4,5-b]pyridin-3-yl)pyridin-2-yl)amino)cyclohexane-1-carboxylic acid I-15 (16.0 mg, 15.2% yield) as a yellow solid and (1r,4r)-4-((5-(2-(2-aminopyridin-3-yl)-5-phenyl-3H-imidazo[4,5-b]pyridin-3-yl)pyridin-2-yl)amino)cyclohexane-1-carboxylic acid I-14 (18.0 mg, 16.9% yield) as a yellow solid.

First Eluting Compound in HPLC: (1r,4r)-4-((5-(2-(2-aminopyridin-3-yl)-5-phenyl-3H-imidazo[4,5-b]pyridin-3-yl)pyridin-2-yl)amino)cyclohexane-1-carboxylic acid I-14: ¹H NMR (400 MHz, DMSO-d₆) δ=8.36 (d, J=8.4 Hz, 1H), 8.26 (s, 1H), 8.18 (dd, J=6.4, 1.6 Hz, 1H), 8.13-8.05 (m, 4H), 7.93 (d, J=8.8 Hz, 1H), 7.55-7.40 (m, 3H), 7.14 (d, J=8.0 Hz, 1H), 6.98 (dd, J=7.2, 6.4 Hz, 1H), 4.00-3.90 (m, 2H), 2.00-1.89 (m, 2H), 1.87-1.72 (m, 2H), 1.72-1.51 (m, 4H). HPLC Rₜ=4.189 min in 8 min chromatography, purity 98.5%. LCMS Rₜ=2.307 min in 4 min chromatography, purity 98.7%, MS ESI calcd. For 505.22 [M+H]⁺ 506.22, found 506.1.

Second Eluting Compound in HPLC: (1s,4s)-4-((5-(2-(2-aminopyridin-3-yl)-5-phenyl-3H-imidazo[4,5-b]pyridin-3-yl)pyridin-2-yl)amino)cyclohexane-1-carboxylic acid 1-15. ¹H NMR (400 MHz, DMSO-d₆) δ=8.35 (d, J=8.0 Hz, 1H), 8.19 (d, J=2.4 Hz, 1H), 8.14-8.01 (m, 5H), 7.86 (dd, J=9.2, 2.0 Hz, 1H), 7.54-7.40 (m, 3H), 7.02-6.91 (m, 2H), 3.65-3.61 (m, 1H), 2.30-2.18 (m, 1H), 2.08-1.89 (m, 4H), 1.53-1.23 (m, 4H). HPLC Rₜ=4.107 min in 8 min chromatography, purity 97.7%. LCMS Rₜ=2.234 min in 4 min chromatography, purity 100%, MS ESI calcd. For 505.22 [M+H]⁺ 506.22, found 506.2.

### Example 16. Synthesis of (1r,4r)-4-(4-(2-(2-aminopyridin-3-yl)-5-phenyl-3H-imidazo[4,5-b]pyridin-3-yl)benzamido)cyclohexane-1-carboxylic acid (Compound I-16)

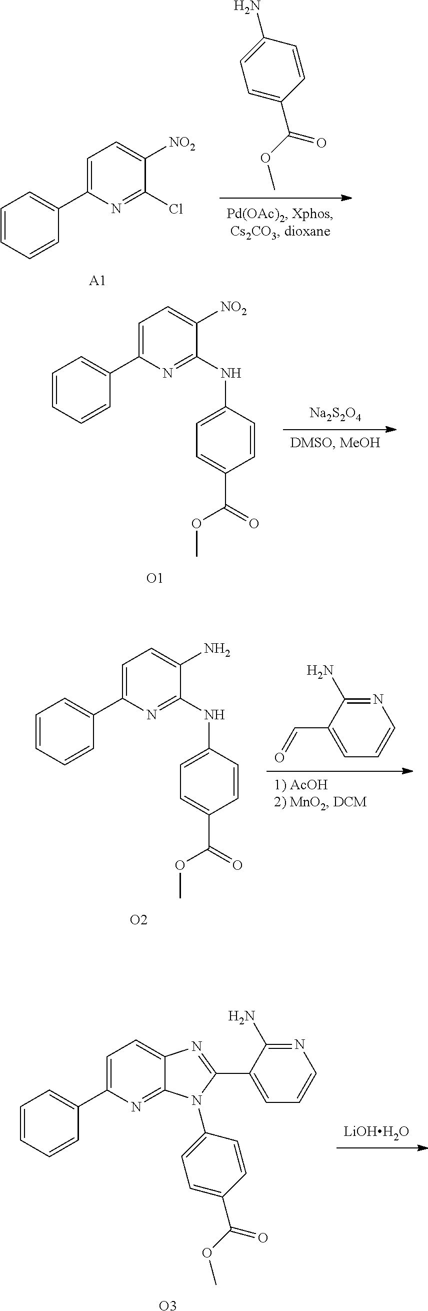

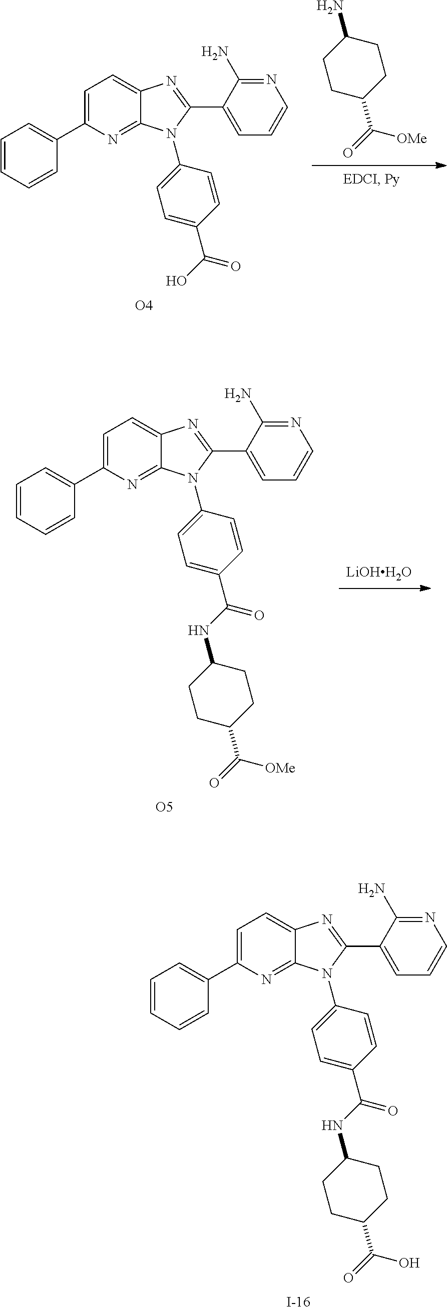

Step 1. To a solution of 2-chloro-3-nitro-6-phenylpyridine A1 (10.0 g, 42.6 mmol) and methyl 4-aminobenzoate (6.44 g, 42.6 mmol) in 1,4-dioxane (150 mL), Cs₂CO₃ (41.7 g, 128 mmol), XPhos (4.06 g, 8.52 mmol) and Pd(OAc)₂ (0.96 g, 4.26 mmol) were added under N₂. The reaction mixture was degassed and purged with N₂ (×3), and the mixture was stirred at 100° C. for 12 hrs under N₂. Water (50 mL) was added, and the resulting mixture was extracted with EtOAc (50 mL×3). The combined organic phase was washed with brine (50 mL), dried over anhydrous Na₂SO₄, filtered and concentrated. The residue was purified by flash silica gel chromatography (eluent with 0-7% of MeOH in DCM) to give methyl 4-((3-nitro-6-phenylpyridin-2-yl)amino)benzoate O1 (11.6 g, 77.9% yield) as a yellow solid. ¹H NMR (DMSO-d₆, 400 MHz) δ_{H}=10.24 (s, 1H), 8.64 (d, J1=8.8 Hz, 1H), 8.17-8.10 (m, 2H), 8.04-7.92 (m, 4H), 7.70 (d, J=8.8 Hz, 1H), 7.59-7.54 (m, 3H), 3.86 (s, 3H).

Step 2. To a solution of methyl 4-((3-nitro-6-phenylpyridin-2-yl)amino)benzoate O1 (11.0 g, 31.5 mmol) in DMSO (50.0 mL) and methanol (50.0 mL), Na₂S₂O₄ (11.0 g, 63.0 mmol) was added under N₂, and the mixture stirred at 100° C. for 12 hrs. The reaction mixture was concentrated, and aq. LiCl solution (10 mL, 3%) was added to the residue. The resulting mixture was extracted with EtOAc (10 mL×3), and the combined organic phase was washed with brine (10 mL), dried over anhydrous Na₂SO₄, filtered and concentrated to give methyl 4-((3-amino-6-phenylpyridin-2-yl)amino)benzoate O2 (8.20 g, crude) as a light yellow oil, which was used in the next step directly. ¹H NMR (DMSO-d₆, 400 MHz) δ_{H}=8.39 (s, 1H), 7.96-7.91 (m, 2H), 7.91-7.88 (m, 2H), 7.84-7.81 (m, 2H), 7.44-7.39 (m, 2H), 7.37 (d, J=8.0 Hz, 114), 7.30-7.24 (m, 1H), 7.06 (d, J=8.0 Hz, 1H), 5.37 (s, 2H), 3.81 (s, 3H).

Step 3. To a solution of methyl 4-((3-amino-6-phenylpyridin-2-yl)amino)benzoate O2 (8.00 g, 25.1 mmol) in acetic acid (50.0 mL), 2-aminopyridine-3-carbaldehyde (4.13 g, 33.8 mmol) was added at 25° C., and the mixture was stirred at 110° C. for 1 hr. The reaction mixture was concentrated directly, the residue was dissolved in DCM (50 mL), MnO₂ (10.9 g, 125 mmol) was subsequently added, and the mixture was stirred at 50° C. for 16 hrs. The reaction mixture was filtered, and the filtrate was concentrated to dryness. The residue was purified by flash silica gel chromatography (eluent with 0-80% of EtOAc in PE) to give methyl 4-(2-(2-aminopyridin-3-yl)-5-phenyl-3H-imidazo[4,5-b]pyridin-3-yl)benzoate O3 (6.20 g, 58.7% yield) as a yellow oil. ¹H NMR (DMSO-d₆, 400 MHz) δ_{H}=8.29 (d, J=8.4 Hz, 1H), 8.15-8.00 (m, 6H), 7.67 (d, J=8.4 Hz, 2H), 7.50-7.37 (m, 3H), 7.26-7.21 (m, 1H), 6.89 (s, 2H), 6.46 (dd, J=4.8, 7.6 Hz, 1H), 3.90 (s, 3H).

Step 4. To a solution of methyl 4-(2-(2-aminopyridin-3-yl)-5-phenyl-3H-imidazo[4,5-b]pyridin-3-yl)benzoate O3 (4.00 g, 9.49 mmol) in water (20.0 mL) and THF (20.0 mL), LiOH·H₂O (1.99 g, 47.5 mmol) was added and the mixture was stirred at 25° C. for 12 hrs. The reaction mixture was concentrated directly and the resulting residue was purified by prep-HPLC (Column: Welch Xtimate C18 150*30 mm*5 μm; mobile phase: [water (HCl)-ACN]; B %, 0%-35%; 25 min) to give 4-(2-(2-aminopyridin-3-yl)-5-phenyl-3H-imidazo[4,5-b]pyridin-3-yl)benzoic acid O4 (2.10 g, 54.3% yield) as a yellow solid. ¹H NMR (DMSO-d₆, 400 MHz) δ_{H} 8.56-8.05 (m, 9H), 7.92-7.88 (m, 1H), 7.72 (d, J=8.4 Hz, 2H), 7.52-7.40 (m, 3H), 6.94-6.89 (m, 1H).

Step 5. To a solution of 4-(2-(2-aminopyridin-3-yl)-5-phenyl-3H-imidazo[4,5-b]pyridin-3-yl)benzoic acid O4 (200 mg, 0.491 mmol) and methyl (1r,4r)-4-aminocyclohexane-1-carboxylate (77.0 mg, 0.491 mmol) in pyridine (10.0 mL), EDCI (113 mg, 0.589 mmol) was added and the mixture was stirred at 25° C. for 12 hrs. Water (5 mL) was added, and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-12% of MeOH in DCM) to give methyl (1r,4r)-4-(4-(2-(2-aminopyridin-3-yl)-5-phenyl-3H-imidazo[4,5-b]pyridin-3-yl)benzamido)cyclohexane-1-carboxylate O5 (140 mg, 65.2% yield) as a light yellow oil. ¹H NMR (400 MHz, DMSO-d₆) δ=8.43 (d, J=8.0 Hz, 1H), 8.28 (d, J=8.4 Hz, 1H), 8.00 (dd, J=7.2, 2.0 Hz, 6H), 7.59 (d, J=8.4 Hz, 2H), 7.47-7.45 (m, 3H), 7.24 (dd, J=7.6, 2.0 Hz, 1H), 6.91 (s, 2H), 6.45 (dd, J=7.6, 4.8 Hz, 1H), 3.80-3.76 (m, 1H), 3.58 (s, 3H), 2.26-2.19 (m, 1H), 1.53-1.40 (m, 8H).

Step 6. To a solution of methyl (1r,4r)-4-(4-(2-(2-aminopyridin-3-yl)-5-phenyl-3H-imidazo[4,5-b]pyridin-3-yl)benzamido)cyclohexane-1-carboxylate O5 (140 mg, 0.256 mmol) in THF (3.00 mL) and water (3.00 mL), LiOH·H₂O (54.0 mg, 1.28 mmol) was added and the mixture was stirred at 25° C. for 4 hrs. The reaction mixture was concentrated directly and the resulting residue was purified by prep-HPLC (Column: Welch Xtimate C18 150*30 mm*5 μm; mobile phase: [water (FA)-ACN]; B %: 14%-54%; 25 min) to give (1r,4r)-4-(4-(2-(2-aminopyridin-3-yl)-5-phenyl-3H-imidazo[4,5-b]pyridin-3-yl)benzamido)cyclohexane-1-carboxylic acid I-16 (4.10 mg, 3.01% yield) as a light yellow solid. ¹H NMR (400 MHz, DMSO-d₆) δ=8.42 (d, J=8.0 Hz, 1H), 8.28 (d, J=8.4 Hz, 1H), 8.08-7.96 (m, 6H), 7.59 (d, J=8.4 Hz, 2H), 7.50-7.44 (m, 2H), 7.43-7.37 (m, 1H), 7.24 (dd, J=7.6, 2.0 Hz, 1H), 6.91 (s, 2H), 6.45 (dd, J=7.6, 4.8 Hz, 1H), 3.82-3.71 (m, 1H), 2.23-2.09 (m, 1H), 2.03-1.84 (m, 4H), 1.51-1.31 (m, 4H). HPLC Rₜ=2.460 min in 8 min chromatography, purity 100%. LCMS Rₜ=1.151 min in 2 min chromatography, purity 99.9%, MS ESI calcd. for 532.22, [M+H]⁺ 533.22, found 532.8.

### Example 17. Synthesis of (1s,4s)-4-(4-(2-(2-aminopyridin-3-yl)-5-phenyl-3H-imidazo[4,5-b]pyridin-3-yl)benzamido)cyclohexane-1-carboxylic acid (Compound I-17)

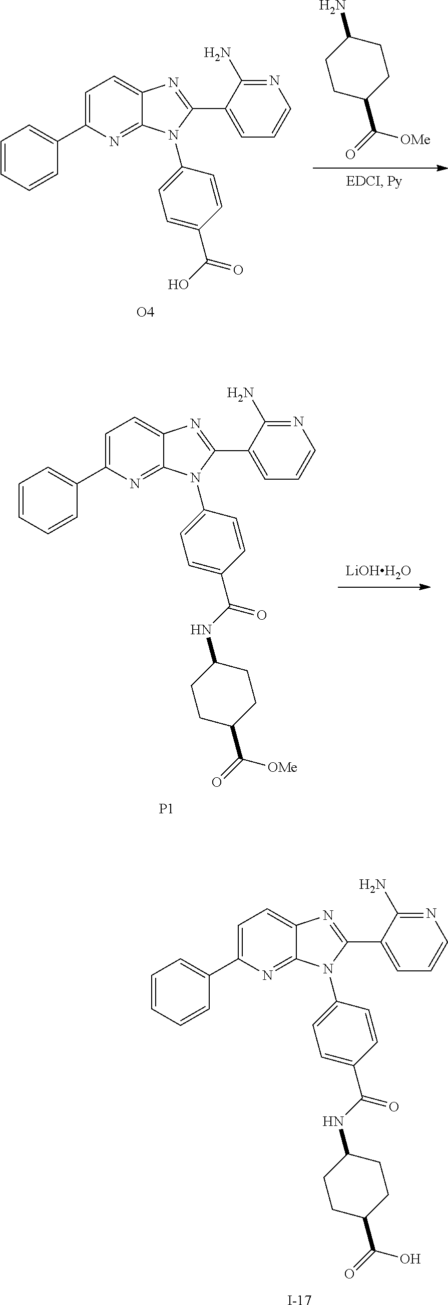

Step 1. To a solution of 4-(2-(2-aminopyridin-3-yl)-5-phenyl-3H-imidazo[4,5-b]pyridin-3-yl)benzoic acid O4 (160 mg, 0.393 mmol) and cis-methyl 4-aminocyclohexanecarboxylate (62.0 mg, 0.393 mmol) in pyridine (10.0 mL), EDCI (113 mg, 0.589 mmol) was added and the mixture was stirred at 25° C. for 12 hrs. Water (5 mL) was added, and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-12% of MeOH in DCM) to give methyl (is, 4s)-4-(4-(2-(2-aminopyridin-3-yl)-5-phenyl-3H-imidazo[4,5-b]pyridin-3-yl)benzamido)cyclohexane-1-carboxylate P1 (140 mg, 65.2% yield) as a light yellow oil. ¹H NMR (DMSO-d₆, 400 MHz) δ_{H}=8.38 (d, J=7.6 Hz, 1H), 8.28 (d, J=8.4 Hz, 1H), 8.07-8.03 (m, 2H), 8.02-7.98 (m, 4H), 7.58 (d, 0.1=8.4 Hz, 2H), 7.47-7.44 (m, 2H), 7.42-7.39 (m, 1H), 7.23 (dd, J=1.6, 7.6 Hz, 1H), 6.92 (s, 2H), 6.45 (dd, J=4.8, 7.6 Hz, 1H), 3.97-3.85 (m, 1H), 3.64 (s, 3H), 2.63-2.58 (m, 1H), 1.63-1.51 (m, 8H).

Step 2. To a solution of methyl (Is, 4s)-4-(4-(2-(2-aminopyridin-3-yl)-5-phenyl-3H-imidazo[4,5-b]pyridin-3-yl)benzamido)cyclohexane-1-carboxylate P1 (140 mg, 0.256 mmol) in THF (3.00 mL) and water (3.00 mL), LiOH·H₂O (54.0 mg, 1.28 mmol) was added and the mixture was stirred at 25° C. for 4 hrs. The reaction mixture was concentrated directly and the resulting residue was purified by prep-HPLC (Column: Welch Xtimate C18 150*30 mm*5 μm; mobile phase: [water (FA)-ACN]; B %: 14%-54%; 25 min) to give (1s,4s)-4-(4-(2-(2-aminopyridin-3-yl)-5-phenyl-3H-imidazo[4,5-b]pyridin-3-yl)benzamido)cyclohexane-1-carboxylic acid 1-17 (46.1 mg, 33.7% yield) as a light yellow solid. ¹H NMR (DMSO-d₆, 400 MHz) δ_{H}=12.18 (s, 1H), 8.40 (d, J=7.6 Hz, 1H), 8.28 (d, J=8.4 Hz, 1H), 8.06-7.99 (m, 6H), 7.58 (d, J=8.4 Hz, 2H), 7.49-7.44 (m, 2H), 7.42-7.37 (m, 1H), 7.23 (dd, J=2.0, 7.6 Hz, 1H), 6.92 (s, 2H), 6.45 (dd, J=4.8, 7.6 Hz, 1H), 3.93-3.85 (m, 1H), 2.08-1.52 (m, 9H). HPLC Rₜ=2.470 min in 8 min chromatography, purity 99.8%. LCMS Rₜ=1.168 min in 2 min chromatography, purity 99.2%, MS ESI calcd. for 532.22, [M+H]⁺ 533.22, found 532.8.

### Example 18. Synthesis of (1s,4s)-4-(5-(2-(2-aminopyridin-3-yl)-5-phenyl-3H-imidazo[4,5-b]pyridin-3-yl)picolinamido)cyclohexane-1-carboxylic acid (Compound I-18)

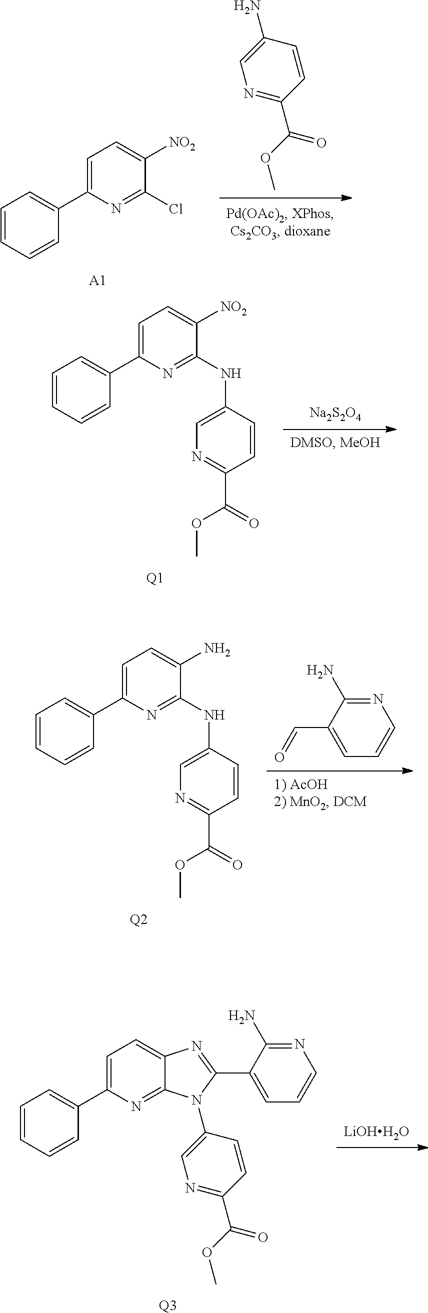

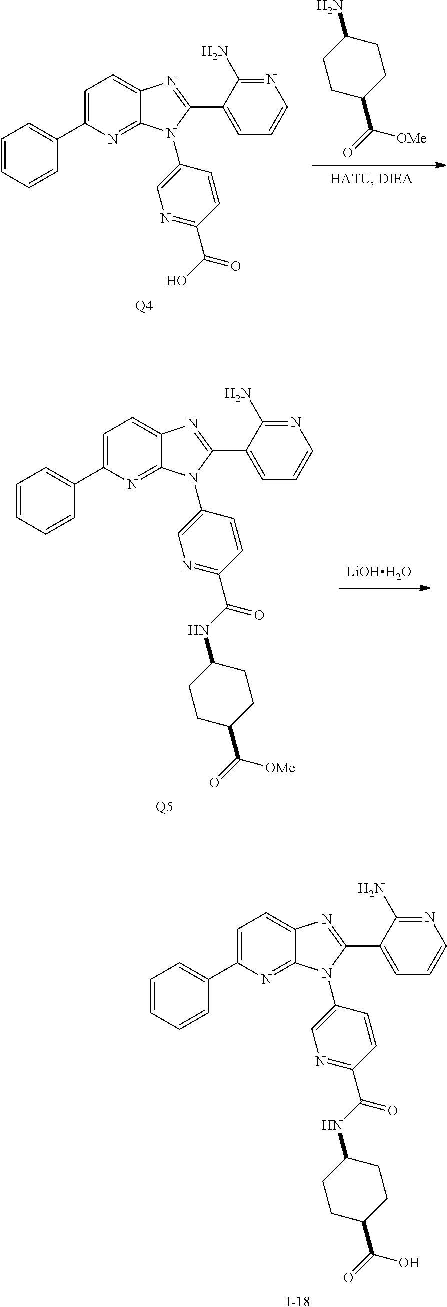

Step 1. To a solution of 2-chloro-3-nitro-6-phenylpyridine A1 (10.0 g, 42.6 mmol) and methyl 5-aminopyridine-2-carboxylate (6.48 g, 42.6 mmol) in 1,4-dioxane (150 mL), Cs₂CO₃ (41.7 g, 128 mmol), XPhos (4.06 g, 8.52 mmol) and Pd(OAc)₂ (0.960 g, 4.26 mmol) were added under N₂. The reaction mixture was degassed and purged with N₂ (×3), and the mixture was stirred at 100° C. for 12 hrs under N₂. Water (50 mL) was added, and the resulting mixture was extracted with EtOAc (50 mL×3). The combined organic phase was washed with brine (50 mL), dried over anhydrous Na₂SO₄, filtered and concentrated. The residue was purified by flash silica gel chromatography (eluent with 0-12% of MeOH in DCM) to give methyl 5-((3-nitro-6-phenylpyridin-2-yl)amino)picolinate Q1 (6.80 g, 42.8% yield) as a yellow solid. ¹H NMR (DMSO-d₆, 400 MHz) δ_{H}=10.31 (s, 1H), 9.11 (d, J=2.4 Hz, 1H), 8.66 (d, J=8.8 Hz, 1H), 8.40 (dd, J=2.4, 8.4 Hz, 1H), 8.16-8.09 (m, 3H), 7.73 (d, J=8.4 Hz, 1H), 7.58-7.55 (m, 3H), 3.89 (s, 3H).

Step 2. To a solution of methyl 5-((3-nitro-6-phenylpyridin-2-yl)amino)picolinate Q1 (5.60 g, 16.0 mmol) in DMSO (40.0 mL) and methanol (40.0 mL), Na₂S₂O₄ (5.57 g, 32.0 mmol) was added under N₂, and the mixture was stirred at 100° C. for 12 hrs. The reaction mixture was concentrated and aq. LiCl (30 mL, 3%) was added to the residue. The resulting mixture was extracted with EtOAc (30 mL×3), and the combined organic phase was washed with brine (30 mL), dried over anhydrous Na₂SO₄, filtered and concentrated to give methyl 5-((3-amino-6-phenylpyridin-2-yl)amino)picolinate Q2 (2.80 g, crude) as a light yellow oil, which was used in the next step directly. ¹H NMR (DMSO-d₆, 400 MHz) 11=9.02 (d, J=2.4 Hz, 1H), 8.59 (s, 1H), 8.30 (dd, J=2.4, 8.8 Hz, 1H), 8.02 (d, J=8.8 Hz, 1H), 7.94-7.91 (m, 2H), 7.76-7.72 (m, 1H), 7.43-7.41 (m, 2H), 7.10 (d, J=8.0 Hz, 1H), 6.94-6.91 (m, 1H), 5.40 (s, 2H), 3.84 (s, 3H).

Step 3. To a solution of methyl 5-((3-amino-6-phenylpyridin-2-yl)amino)picolinate Q2 (2.50 g, 7.80 mmol) in acetic acid (20.0 mL), 2-aminopyridine-3-carbaldehyde (1.05 g, 8.58 mmol) was added and the mixture stirred at 110° C. for 1 hr. The reaction mixture was concentrated directly. DCM (40.0 mL) and MnO₂ (6.80 g, 78.0 mmol) were then added to the residue and the mixture was stirred at 50° C. for 16 hrs. The reaction mixture was filtered, and the filtrate was concentrated to dryness. The residue was purified by flash silica gel chromatography (eluent with 0-10% of MeOH in DCM) to give methyl 5-(2-(2-aminopyridin-3-yl)-5-phenyl-3H-imidazo[4,5-b]pyridin-3-yl)picolinate Q3 (1.50 g, 39.6% yield) as a yellow oil. ¹H NMR (DMSO-d₆, 400 MHz) δ_{H}=8.89 (d, J=2.0 Hz, 1H), 8.32 (d, J=8.4 Hz, 1H), 8.24 (d, J=8.4 Hz, 1H), 8.16-8.13 (m, 1H), 8.09-8.07 (m, 2H), 8.06-8.03 (m, 2H), 7.49-7.46 (m, 2H), 7.42-7.38 (m, 2H), 6.67 (s, 2H), 6.54 (dd, J=4.8, 7.6 Hz, 1H), 3.92 (s, 3H).

Step 4. To a solution of methyl 5-(2-(2-aminopyridin-3-yl)-5-phenyl-3H-imidazo[4,5-b]pyridin-3-yl)picolinate Q3 (1.50 g, 3.55 mmol) in water (20.0 mL) and THF (20.0 mL), LiOH·H₂O (0.750 g, 17.8 mmol) was added and the mixture was stirred at 25° C. for 12 hrs. The reaction mixture was concentrated directly and the resulting residue was purified by prep-HPLC (Column: Welch Xtimate C18 150*30 mm*5 μm; mobile phase: [water (HCl)-ACN]; B %: 0%-35%; 25 min) to give 5-(2-(2-aminopyridin-3-yl)-5-phenyl-3H-imidazo[4,5-b]pyridin-3-yl)picolinic acid Q4 (400 mg, 27.6% yield) as a light yellow solid. ¹H NMR (DMSO-d₆, 400 MHz) δ_{H}=8.94 (d, J=2.0 Hz, 1H), 8.40 (d, J=8.4 Hz, 1H), 8.29-7.87 (m, 9H), 7.52-7.41 (m, 31H), 6.93-6.86 (m, 1H).

Step 5. To a solution of 5-[2-(2-amino-3-pyridyl)-5-phenyl-imidazo[4,5-b]pyridin-3-yl]pyridine-2-carboxylic acid Q4 (100 mg, 0.245 mmol) and cis-methyl 4-aminocyclohexanecarboxylate (38.0 mg, 0.245 mmol) in DCM (5.00 mL), DIEA (95.0 mg, 0.735 mmol) and HATU (112 mg, 0.294 mmol) were added and the mixture was stirred at 25° C. for 12 hrs. Water (5 mL) was added, and the resulting mixture was extracted with EtOAc (10 mL 3). The combined organic phase was washed with brine (5 mL), dried over anhydrous Na₂SO₄, filtered and concentrated. The residue was purified by flash silica gel chromatography (eluent with 0-12% of MeOH in DCM) to give cis-methyl 4-[[5-[2-(2-amino-3-pyridyl)-5-phenyl-imidazo[4,5-b]pyridin-3-yl]pyridine-2-carbonyl]amino]cyclohexanecarboxylate Q5 (70.0 mg, 52.2% yield) as a yellow oil. ¹H NMR (400 MHz, DMSO-d₆) δ_{H}=8.81 (d, J=2.4 Hz, 1H), 8.56 (d, J=8.4 Hz, 1H), 8.31 (d, J=8.4 Hz, 1H), 8.19-8.17 (m, 1H), 8.15-8.11 (m, 1H), 8.08-8.04 (m, 4H), 7.50-7.46 (m, 2H), 7.43-7.38 (m, 2H), 6.68 (s, 2H), 6.54 (dd, J=4.8, 7.6 Hz, 1H), 3.93-3.88 (m, 1H), 3.63 (s, 3H), 2.64-2.58 (m, 1H), 1.73-1.58 (m, 8H).

Step 6. To a solution of cis-methyl 4-[[5-[2-(2-amino-3-pyridyl)-5-phenyl-imidazo[4,5-b]pyridin-3-yl]pyridine-2-carbonyl]amino]cyclohexanecarboxylate Q5 (70.0 mg, 0.128 mmol) in THF (2.00 mL), LiOH·H₂O (16.0 mg, 0.383 mmol) and water (0.500 mL) were added and the mixture was stirred at 25° C. for 12 hrs. The reaction mixture was filtered, and the filtrate was concentrated to dryness. The residue was purified by prep-HPLC (Column: Xtimate C18 150*40 mm*10 μm; mobile phase: [water (NH₄HCO₃)-ACN]; B %: 2%-42%, 25 min) to give (1s,4s)-4-(5-(2-(2-aminopyridin-3-yl)-5-phenyl-3H-imidazo[4,5-b]pyridin-3-yl)picolinamido)cyclohexane-1-carboxylic acid I-18 (26.6 mg, 38.6% yield) as a white solid. ¹H NMR (400 MHz, DMSO-d₆) δ=8.82 (d, J=2.0 Hz, 1H), 8.56 (d, J=8.0 Hz, 1H), 8.31 (d, J=8.4 Hz, 1H), 8.21-8.00 (m, 6H), 7.52-7.36 (m, 4H), 6.68 (s, 2H), 6.53 (dd, J=4.8, 7.6 Hz, 1H), 3.90 (dd, J=4.4, 7.6 Hz, 1H), 2.48-2.45 (m, 1H), 1.99-1.88 (m, 2H), 1.75-1.53 (m, 6H). HPLC Rₜ=2.844 min in 8 min chromatography, purity 98.9%. LCMS Rₜ=0.792 min in 2.0 min chromatography, purity 98.7%, MS ESI calcd. for 533.22 [M+H]⁺ 534.22, found 534.2.

### Example 19. Synthesis of (1r,4r)-4-(5-(2-(2-aminopyridin-3-yl)-5-phenyl-3H-imidazo[4,5-b]pyridin-3-yl)picolinamido)cyclohexane-1-carboxylic acid (Compound I-19)

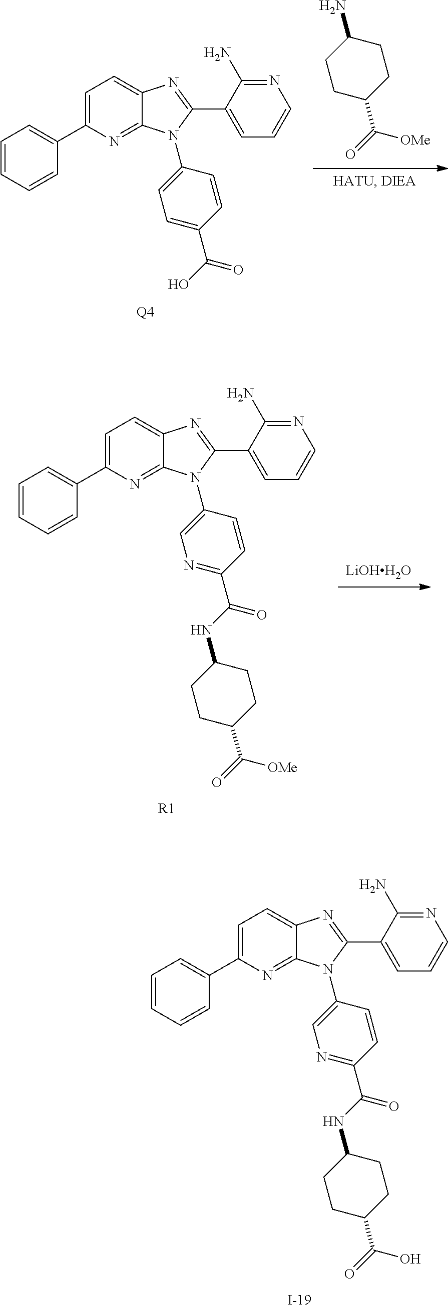

Step 1. To a solution of 5-[2-(2-amino-3-pyridyl)-5-phenyl-imidazo[4,5-b]pyridin-3-yl]pyridine-2-carboxylic acid Q4 (100 mg, 0.245 mmol) and trans-methyl 4-aminocyclohexanecarboxylate (38.0 mg, 0.245 mmol) in DCM (5.00 mL), DIEA (95.0 mg, 0.735 mmol) and HATU (112 mg, 0.294 mmol) were added and the mixture was stirred at 25° C. for 12 hrs. Water (5 mL) was added, and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-12% of MeOH in DCM) to give trans-methyl 4-[[5-[2-(2-amino-3-pyridyl)-5-phenyl-imidazo[4,5-b]pyridin-3-yl]pyridine-2-carbonyl]amino]cyclohexanecarboxylate R1 (70.0 mg, 52.2% yield) as a yellow solid. ¹H NMR (DMSO-d₆, 400 MHz) δ=8.82-8.79 (m, 1H), 8.67 (d, J=8.4 Hz, 1H), 8.31 (d, J=8.4 Hz, 1H), 8.20-8.18 (m, 1H), 8.15-8.12 (m, 1H), 8.08-8.04 (m, 4H), 7.51-7.46 (m, 2H), 7.43-7.39 (m, 2H), 6.67 (s, 2H), 6.54 (dd, J=4.8, 7.6 Hz, 1H), 3.83-3.75 (m, 1H), 3.60 (s, 3H), 2.26 (td, J=3.6, 7.8 Hz, 1H), 1.96 (d, J=11.2 Hz, 2H), 1.88 (d, J=10.0 Hz, 2H), 1.52-1.42 (m, 4H).

Step 2. To a solution of trans-methyl 4-[[5-[2-(2-amino-3-pyridyl)-5-phenyl-imidazo[4,5-b]pyridin-3-yl]pyridine-2-carbonyl]amino]cyclohexanecarboxylate R1 (70.0 mg, 0.128 mmol) in THF (2.00 mL), LiOH·H₂O (16.0 mg, 0.383 mmol) and water (0.500 mL) were added and the mixture was stirred at 25° C. for 12 hrs. The reaction mixture was filtered, and the filtrate was concentrated to dryness. The residue was purified by prep-HPLC (Column: Xtimate C18 150*40 mm*10 μm; mobile phase: [water (NH₄HCO₃)-ACN]; B %: 2%-42%, 25 min) to give (1r,4r)-4-(5-(2-(2-aminopyridin-3-yl)-5-phenyl-3H-imidazo[4,5-b]pyridin-3-yl)picolinamido) cyclohexane-1-carboxylic acid 1-19 (28.9 mg, 41.9% yield) as a white solid. ¹H NMR (DMSO-d₆, 400 M Hz) 81=8.81 (d, J=2.0 Hz, 1H), 8.64 (d, J=8.4 Hz, 1H), 8.31 (d, J=8.4 Hz, 1H), 8.23-8.00 (m, 6H), 7.52-7.37 (m, 4H), 6.67 (s, 2H), 6.54 (dd, J=4.8, 7.6 Hz, 1H), 3.83-3.74 (m, 1H), 2.19-2.09 (m, 1H), 1.99-1.84 (m, 4H), 1.57-1.36 (m, 4H). HPLC Rₜ=2.848 min in 8 min chromatography, purity 98.9%. LCMS Rₜ=0.792 min in 2.0 min chromatography, purity 98.8%, MS ESI calcd. for 533.22 [M+H]⁺ 534.22, found 534.3.

### Example 20. Synthesis of 1-(4-(2-(2-aminopyridin-3-yl)-5-phenyl-3H-imidazo[4,5-b]pyridin-3-yl)benzoyl)piperidine-4-carboxylic acid (Compound I-20)

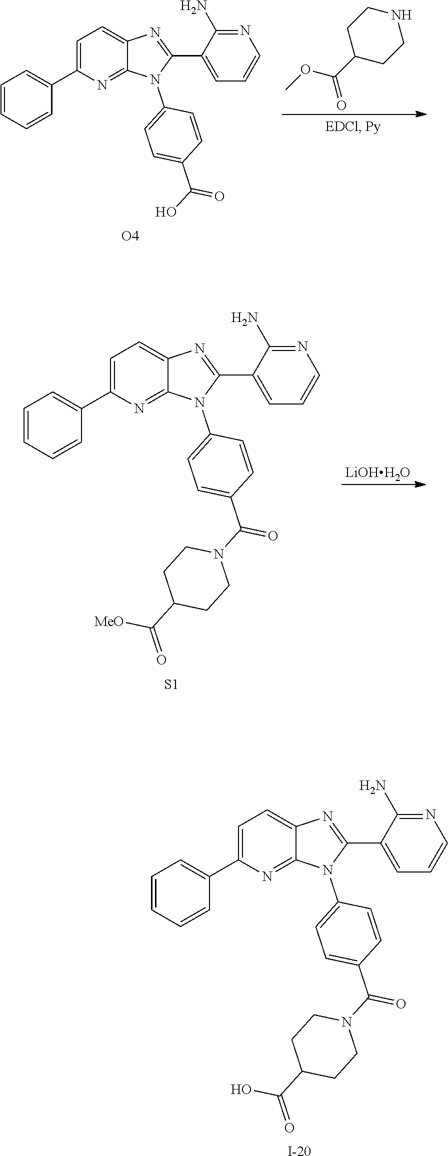

Step 1. To a solution of 4-[2-(2-amino-3-pyridyl)-5-phenyl-imidazo[4,5-b]pyridin-3-yl]benzoic acid O4 (100 mg, 0.245 mmol) and methyl piperidine-4-carboxylate (35.0 mg, 0.245 mmol) in pyridine (5.00 mL), EDCI (70.0 mg, 0.368 mmol) was added and the mixture was stirred at 25° C. for 12 hrs. Water (5 mL) was added, and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent of 0-12% of MeOH in DCM) to give methyl 1-(4-(2-(2-aminopyridin-3-yl)-5-phenyl-3H-imidazo[4,5-b]pyridin-3-yl)benzoyl)piperidine-4-carboxylate S1 (70.0 mg, 53.6% yield) as a light yellow oil. ¹H NMR (DMSO-d₆, 400 MHz) δ_{H}=8.28 (d, J=8.4 Hz, 1H), 8.07-7.99 (m, 4H), 7.57 (s, 4H), 7.50-7.44 (m, 2H), 7.42-7.37 (m, 1H), 7.22 (dd, J=1.6, 7.6 Hz, 1H), 6.92 (s, 2H), 6.44 (dd, J=4.8, 7.6 Hz, 1H), 3.63 (s, 3H), 3.36-3.35 (m, 2H), 3.30-3.30 (m, 2H), 2.52-2.52 (m, 1H), 1.95-1.81 (m, 2H), 1.63-1.53 (m, 2H).

Step 2. To a solution of methyl 1-(4-(2-(2-aminopyridin-3-yl)-5-phenyl-3H-imidazo[4,5-b]pyridin-3-yl)benzoyl)piperidine-4-carboxylate S1 (70.0 mg, 0.132 mmol) in THF (3.00 mL) and water (3.00 mL), LiOH·H₂O (39.0 mg, 0.939 mmol) was added and the mixture was stirred at 25° C. for 4 hrs. The reaction mixture was concentrated directly and the resulting residue was purified by prep-HPLC (Column: Welch Xtimate C18 150*30 mm*5 μm; mobile phase: [water (FA)-ACN]; B %: 0%-35%; 25 min) to give 1-(4-(2-(2-aminopyridin-3-yl)-5-phenyl-3H-imidazo[4,5-b]pyridin-3-yl)benzoyl)piperidine-4-carboxylic acid 1-20 (33.2 mg, 48.7% yield) as a white solid. ¹H NMR (DMSO-d₆, 400 MHz) δ_{H}=12.32 (s, 1H), 8.28 (d, J=8.4 Hz, 1H), 8.08-7.99 (m, 4H), 7.57 (s, 4H), 7.50-7.37 (m, 3H), 7.22 (dd, J=1.6, 7.6 Hz, 1H), 6.94 (s, 2H), 6.44 (dd, 0.1=4.8, 7.6 Hz, 1H), 4.55-4.13 (m, 1H), 3.71-3.46 (m, 1H), 3.21-2.94 (m, 2H), 2.60-2.53 (m, 1H), 2.04-1.74 (m, 2H), 1.65-1.45 (m, 2H). HPLC Rₜ=2.294 min in 8.0 min chromatography, purity 99.9%. LCMS Rₜ=1.096 min in 2 min chromatography, purity 99.8%, MS ESI calcd. for 518.21, [M+H]⁺ 519.21, found 518.8.

### Example 21. Synthesis of 1-(4-(2-(2-aminopyridin-3-yl)-5-phenyl-3H-imidazo[4,5-b]pyridin-3-yl)benzoyl)piperidine-3-carboxylic acid (Compound I-21)

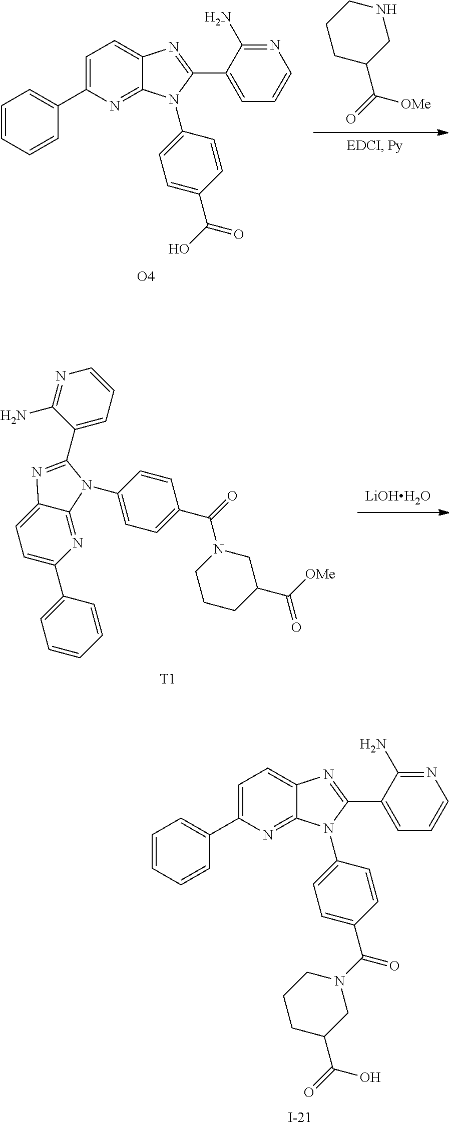

Step 1. To a solution of 4-[2-(2-amino-3-pyridyl)-5-phenyl-imidazo[4,5-b]pyridin-3-yl]benzoic acid O4 (160 mg, 0.393 mmol) and methyl piperidine-3-carboxylate (56.0 mg, 0.393 mmol) in pyridine (10.0 mL), EDCI (113 mg, 0.589 mmol) was added and the mixture was stirred at 25° C. for 12 hrs. Water (5 mL) was added to the residue, and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-13% of MeOH in DCM) to give methyl 1-[4-[2-(2-amino-3-pyridyl)-5-phenyl-imidazo[4,5-b]pyridin-3-yl]benzoyl]piperidine-3-carboxylate T1 (280 mg, 73.6% yield) as a light yellow oil. LCMS Rₜ=0.565 min in 2 min chromatography, Xtimate C18 2.1*30 mm, 3 μm, purity 55.5%, MS ESI calcd. for 532.22 [M+H]⁺ 533.22, found 533.3.

Step 2. To a solution of methyl 1-[4-[2-(2-amino-3-pyridyl)-5-phenyl-imidazo[4,5-b]pyridin-3-yl]benzoyl]piperidine-3-carboxylate T1 (509 mg, 0.526 mmol) in THF (3.00 mL) and water (3.00 mL), LiOH·H₂O (110 mg, 2.63 mmol) was added and the mixture was stirred at 25° C. for 4 hrs. The reaction mixture was concentrated directly and the resulting residue was purified by prep-HPLC (Column: Welch Xtimate C18 200*40 mm*7 μm: mobile phase: [water (FA)-ACN], B %: 4%-44%; 25 min) to give 1-[4-[2-(2-amino-3-pyridyl)-5-phenyl-imidazo[4,5-b]pyridin-3-yl]benzoyl]piperidine-3-carboxylic acid I-21 (21.0 mg, 7.74% yield) as a light yellow solid ¹H NMR (DMSO-d₆, 400 MHz) δ_{H}=8.29 (d, J=8.4 Hz, 1H), 8.09-7.96 (m, 4H), 7.59 (s, 4H), 7.52-7.45 (m, 2H), 7.41 (d, J=7.2 Hz, 1H), 7.29-7.13 (m, 1H), 7.10-6.88 (m, 2H), 6.51-6.33 (m, 1H), 4.55-4.07 (m, 1H), 3.78-3.54 (m, 1H), 3.21-2.97 (m, 3H), 2.10-1.93 (m, 1H), 1.80-1.44 (m, 3H). HPLC Rₜ=2.353 min in 8.0 min chromatography, purity 100%. LCMS Rₜ=1.137 min in 2 min chromatography, purity 99.9%, MS ESI calcd. for 518.21 [M+H]⁺ 519.21, found 518.7.

### Example 22. Synthesis of 1-(5-(2-(2-aminopyridin-3-yl)-5-phenyl-3H-imidazo[4,5-b]pyridin-3-yl)picolinoyl)piperidine-4-carboxylic acid (Compound I-22)

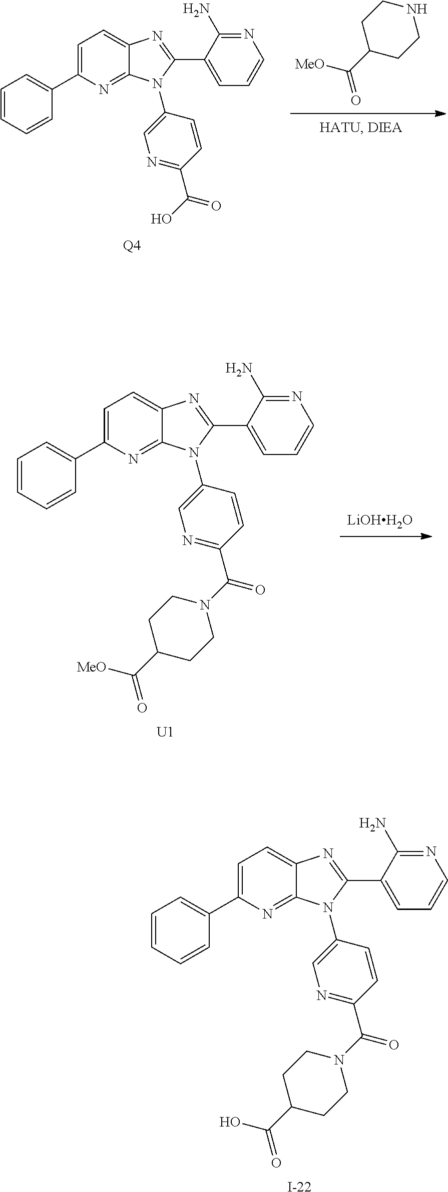

Step 1. To a solution of 5-(2-(2-aminopyridin-3-yl)-5-phenyl-3H-imidazo[4,5-b]pyridin-3-yl)picolinic acid Q4 (100 mg, 0.245 mmol) and methyl piperidine-4-carboxylate (35.0 mg, 0.245 mmol) in DCM (5.00 mL), DIEA (95.0 mg, 0.735 mmol) and HATU (112 mg, 0.294 mmol) were added and the mixture was stirred at 25° C. for 12 hrs. Water (5 mL) was added, and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-12% MeOH in DCM) to give methyl 1-(5-(2-(2-aminopyridin-3-yl)-5-phenyl-3H-imidazo[4,5-b]pyridin-3-yl)picolinoyl)piperidine-4-carboxylate U1 (100 mg, 76.5% yield) as a light yellow oil. ¹H NMR (DMSO-d₆, 400 MHz) δ_{H}=8.70 (d, J=2.4 Hz, 1H), 8.30 (d, J=8.4 Hz, 1H), 8.12 (dd, J=2.4, 8.4 Hz, 1H), 8.08-8.01 (m, 4H), 7.78 (d, J=8.4 Hz, 1H), 7.51-7.45 (m, 2H), 7.44-7.40 (m, 1H), 7.38-7.34 (m, 1H), 6.72 (s, 2H), 6.51 (dd, J=4.8, 7.6 Hz, 1H), 3.62 (s, 3H), 3.49-3.48 (m, 2H), 3.37-3.27 (m, 2H), 2.48-2.47 (m, 1H), 1.95-1.93 (m, 2H), 1.81-1.80 (m, 2H).

Step 2. To a solution of methyl 1-(5-(2-(2-aminopyridin-3-yl)-5-phenyl-3H-imidazo[4,5-b]pyridin-3-yl)picolinoyl)piperidine-4-carboxylate U1 (100 mg, 0.187 mmol) in THF (3.00 mL) and water (3.00 mL), LiOH·H₂O (39.0 mg, 0.937 mmol) was added and the mixture was stirred at 25° C. for 4 hrs. The reaction mixture was concentrated directly and the resulting residue was purified by prep-HPLC (Column: Welch Xtimate C18 150*30 mm*5 μm; mobile phase: [water (FA)-ACN]; B %: 0%-35%; 25 min) to give 1-(5-(2-(2-aminopyridin-3-yl)-5-phenyl-3H-imidazo[4,5-b]pyridin-3-yl)picolinoyl)piperidine-4-carboxylic acid 1-22 (41.3 mg, 42.3% yield) as a light yellow solid. ¹H NMR (DMSO-d₆, 400 MHz) δ_{H}=12.31 (s, 1H), 8.71 (d, J=2.0 Hz, 1H), 8.32 (d, J=8.4 Hz, 1H), 8.14-8.02 (m, 5H), 7.78 (d, J=8.4 Hz, 1H), 7.51-7.38 (m, 4H), 6.78 (s, 2H), 6.53 (dd, J=4.8, 7.6 Hz, 1H), 4.42-4.31 (m, 1H), 3.68-3.57 (m, 1H), 3.21-3.12 (m, 1H), 3.06-2.97 (m, 1H), 2.63-2.54 (m, 1H), 1.99-1.78 (m, 2H), 1.63-1.48 (m, 2H). HPLC Rₜ=2.092 min in 8.0 min chromatography, purity 96.5%. LCMS Rₜ=1.032 min in 2.0 min chromatography, purity 99.7%, MS ESI calcd. for 519.20, [M+H]⁺ 520.20, found 519.8.

### Example 23. Synthesis of 1-(5-(2-(2-aminopyridin-3-yl)-5-phenyl-3H-imidazo[4,5-b]pyridin-3-yl)picolinoyl)-4-methylpiperidine-4-carboxylic acid (Compound I-23)

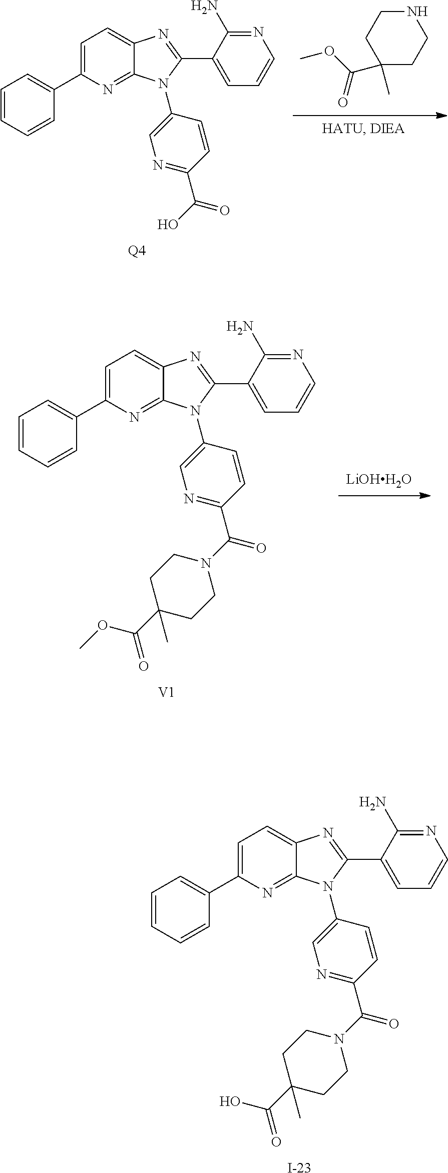

Step 1. To a solution of 5-(2-(2-aminopyridin-3-yl)-5-phenyl-3H-imidazo[4,5-b]pyridin-3-yl)picolinic acid Q4 (50.0 mg, 0.122 mmol) and methyl 3,3-dimethylpiperidine-4-carboxylate (21.0 mg, 0.122 mmol) in DCM (5.00 mL), DIEA (47.0 mg, 0.367 mmol) and HATU (70.0 mg, 0.184 mmol) were added and the mixture was stirred at 25° C. for 12 hrs. Water (5 mL) was added, and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-12% of MeOH in DCM) to give methyl 1-(5-(2-(2-aminopyridin-3-yl)-5-phenyl-3H-imidazo[4,5-b]pyridin-3-yl)picolinoyl)-4-methylpiperidine-4-carboxylate V1 (50 mg, 72.7% yield) as a light yellow oil. ¹H NMR (DMSO-d₆, 400 MHz) δ_{H}=8.70 (d, J=2.4 Hz, 1H), 8.31 (d, J=8.4 Hz, 1H), 8.14-8.03 (m, 5H), 7.78 (d, J=8.4 Hz, 1H), 7.51-7.36 (m, 4H), 6.73 (s, 2H), 6.52 (dd, J=4.8, 7.6 Hz, 1H), 4.12-4.04 (m, 1H), 3.57-3.47 (m, 4H), 3.20-3.16 (m, 2H), 2.09-2.03 (m, 2H), 1.52-1.48 (m, 2H), 1.22 (s, 3H).

Step 2. To a solution of methyl 1-(5-(2-(2-aminopyridin-3-yl)-5-phenyl-3H-imidazo[4,5-b]pyridin-3-yl)picolinoyl)-4-methylpiperidine-4-carboxylate V1 (50.0 mg, 0.0913 mmol) in THF (3.00 mL) and water (3.00 mL), LiOH·H₂O (19.0 mg, 0.457 mmol) was added and the mixture was stirred at 25° C. for 4 hrs. The reaction mixture was concentrated directly and the resulting residue was purified by prep-HPLC (Column. Welch Xtimate C18 150*30 mm*5 μm; mobile phase: [water (FA)-ACN]; B %: 0%-35%; 25 min) to give 1-(5-(2-(2-aminopyridin-3-yl)-5-phenyl-3H-imidazo[4,5-b]pyridin-3-yl)picolinoyl)-4-methylpiperidine-4-carboxylic acid I-23 (13.4 mg, 27.3% yield) as a light yellow solid. ¹H NMR (DMSO-d₆, 400 MHz) δ_{H}=12.52 (s, 1H), 8.70 (d, J=2.4 Hz, 1H), 8.32 (d, J=8.4 Hz, 1H), 8.16-8.01 (m, 5H), 7.78 (d, J=8.4 Hz, 1H), 7.52-7.35 (m, 4H), 6.76 (s, 2H), 6.53 (dd, J=4.8, 7.6 Hz, 1H), 4.16-4.06 (m, 1H), 3.57-3.48 (m, 1H), 3.21-3.12 (m, 2H), 2.10-1.88 (m, 2H), 1.49-1.36 (m, 2H), 1.19 (s, 3H). HPLC Rₜ=2.332 min in 8 min chromatography, purity 99.3%. LCMS Rₜ=1.116 min in 2 min chromatography, purity 99.3%, MS ESI calcd. for 533.22, [M+H]⁺ 534.22, found 533.9.

### Example 24. Synthesis of I-(5-(2-(2-aminopyridin-3-yl)-5-phenyl-3H-imidazo[4,5-b]pyridin-3-yl)picolinoyl)-3,3-dimethylpiperidine-4-carboxylic acid (Compound I-24)

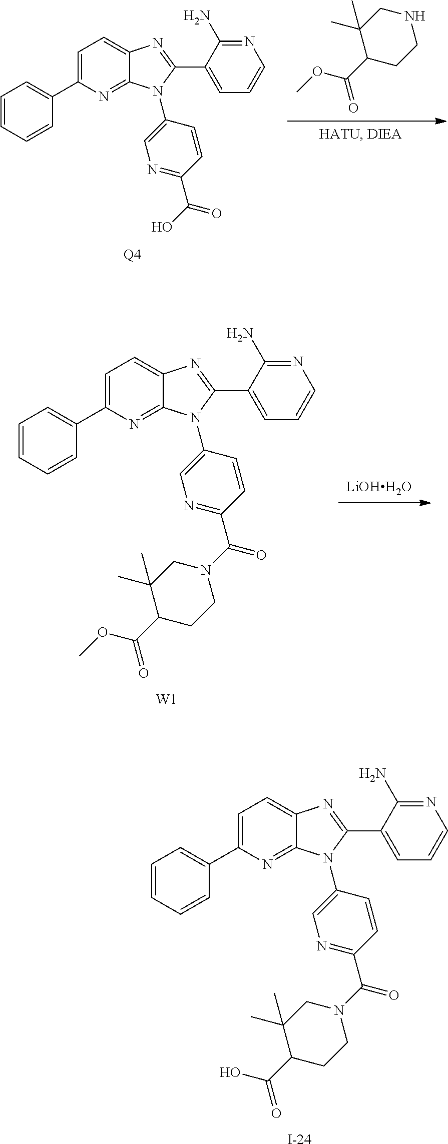

Step 1. To a solution of 5-(2-(2-aminopyridin-3-yl)-5-phenyl-3H-imidazo[4,5-b]pyridin-3-yl)picolinic acid Q4 (50.0 mg, 0.122 mmol) and methyl 3,3-dimethylpiperidine-4-carboxylate (21.0 mg, 0.122 mmol) in DCM (5.00 mL), DIEA (47.0 mg, 0.367 mmol) and HATU (70.0 mg, 0.184 mmol) were added and the mixture was stirred at 25° C. for 12 hrs. Water (5 mL) was added, and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-12% of MeOH in DCM) to give methyl 1-(5-(2-(2-aminopyridin-3-yl)-5-phenyl-3H-imidazo[4,5-b]pyridin-3-yl)picolinoyl)-3,3-dimethylpiperidine-4-carboxylate W1 (50.0 mg, 72.7% yield) as a light yellow oil. ¹H NMR (DMSO-d₆, 400 MHz) δ_{H}=8.76-8.65 (m, 1H), 8.31 (d, J=8.4 Hz, 1H), 8.14-8.11 (m, 1H), 8.09-8.02 (m, 4H), 7.81-7.74 (m, 1H), 7.52-7.34 (m, 4H), 6.75 (d, J=10.4 Hz, 2H), 6.55-6.44 (m, 1H), 3.62-3.60 (m, 5H), 3.14-3.12 (m, 2H), 2.53-2.52 (m, 1H), 1.30-1.20 (m, 8H).

Step 2. To a solution of methyl 1-(5-(2-(2-aminopyridin-3-yl)-5-phenyl-3H-imidazo[4,5-b]pyridin-3-yl)picolinoyl)-3,3-dimethylpiperidine-4-carboxylate W1 (50.0 mg, 0.0890 mmol) in THF (3.00 mL) and water (3.00 mL), LiOH·H₂O (19.0 mg, 0.445 mmol) was added and the mixture was stirred at 25° C. for 4 hrs. The reaction mixture was concentrated directly and the resulting residue was purified by prep-HPLC (Column: Welch Xtimate C18 150*30 mm*5 μm; mobile phase: [water (FA)-ACN]; B %: 0%-35%; 25 min) to give 1-(5-(2-(2-aminopyridin-3-yl)-5-phenyl-3H-imidazo[4,5-b]pyridin-3-yl)picolinoyl)-3,3-dimethylpiperidine-4-carboxylic acid 1-24 (18.1 mg, 37.0% yield) as a light yellow solid. ¹H NMR (DMSO-d₆, 400 MHz) δ_{H}=12.26 (s, 1H), 8.71 (d, 1H), 8.32 (d, J=8.4 Hz, 1H), 8.16-8.01 (m, 5H), 7.77 (t, J=8.0 Hz, 1H), 7.52-7.35 (m, 4H), 6.87-6.70 (m, 2H), 6.57-6.44 (m, 1H), 4.53-3.58 (m, 1H), 3.21 (d, J=13.6 Hz, 1H), 3.15-2.75 (m, 2H), 2.43-2.34 (m, 1H), 1.91-1.58 (m, 2H), 1.11-0.74 (m, 6H). HPLC Rₜ=2.341 min in 8 min chromatography, purity 99.8%. LCMS Rₜ=1.116 min in 2 min chromatography, purity 9944%, MS ESI calcd. for 547.23, [M+H]⁺ 548.23, found 547.9.

### Example 25. Synthesis of 1-(5-(2-(2-aminopyridin-3-yl)-5-phenyl-3H-imidazo[4,5-b]pyridin-3-yl)pyridin-2-yl)pyrrolidine-3-carboxylic acid (Compound I-25)

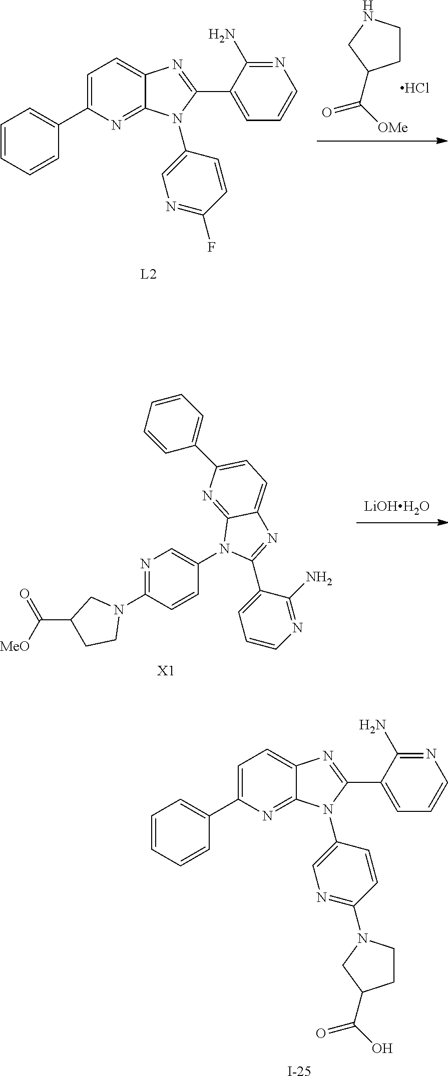

Step 1. To a stirred solution of 3-[3-(6-fluoro-3-pyridyl)-5-phenyl-imidazo[4,5-b]pyridin-2-yl]pyridin-2-amine L2 (100 mg, 0.260 mmol) and methyl pyrrolidine-3-carboxylate (68.0 mg, 0.520 mmol) in DMSO (2.00 mL), DIEA (0.240 mL, 1.31 mmol) was added and the reaction mixture was stirred at 80° C. under N₂ for 16 hrs. The reaction mixture was diluted with EtOAc (15 mL), washed with water (15 mL) and brine (15 mL×2), dried over anhydrous sodium sulfate, filtered and the filtrate was concentrated to afford methyl 1-[5-[2-(2-amino-3-pyridyl)-5-phenyl-imidazo[4,5-b]pyridin-3-yl]-2-pyridyl]pyrrolidine-3-carboxylate X1 (100 mg, 77.8% yield) as a yellow solid. ¹H NMR (400 MHz, CDCl₃) δ=8.30 (d, J=2.4 Hz, 1H), 8.15-8.00 (m, 4H), 7.79 (d, J=8.4 Hz, 1H), 7.50-7.29 (m, 5H), 6.59 (s, 2H), 6.51-6.45 (m, 2H), 3.90-3.79 (m, 2H), 3.76 (s, 3H), 3.72-3.67 (m, 1H), 3.62-3.51 (m, 1H), 3.34-3.22 (m, 1H), 2.41-2.30 (m, 2H).

Step 2. To a stirred solution of methyl 1-[5-[2-(2-amino-3-pyridyl)-5-phenyl-imidazo[4,5-b]pyridin-3-yl]-2-pyridyl]pyrrolidine-3-carboxylate X1 (100 mg, 0.200 mmol) in THF (1.00 mL) and water (1.00 mL), LiOH·H₂O (16.0 mg, 0.410 mmol) was added and the reaction mixture was stirred at 25° C. under N₂ for 16 hrs. The reaction mixture was adjusted to pH ~5 with IN HCl solution and concentrated to afford a residue. The crude product was purified by prep-HPLC (column: Welch Xtimate C18 150×30 mm×5 μm, method: water (FA)-ACN, begin B: 18, end B: 48) to afford 1-[5-[2-(2-amino-3-pyridyl)-5-phenyl-imidazo[4,5-b]pyridin-3-yl]-2-pyridyl]pyrrolidine-3-carboxylic acid I-25 (43.0 mg, 43.9% yield) as a white solid. ¹H NMR (400 MHz, DMSO-d₆) δ=12.52 (br s, 1H), 8.25 (d, J=8.4 Hz, 1H), 8.17 (d, J=2.4 Hz, 1H), 8.07-7.94 (m, 4H), 7.62 (dd, J=2.8, 8.8 Hz, 1H), 7.51-7.44 (m, 2H), 7.43-7.36 (m, 2H), 6.88 (s, 2H), 6.61 (d, J=8.8 Hz, 1H), 6.51 (dd, J=4.8, 7.6 Hz, 1H), 3.73-3.41 (m, 4H), 3.27-3.17 (m, 1H), 2.30-2.11 (m, 2H). HPLC Rₜ=2.857 min in 8 min chromatography, purity 99.0%. LCMS Rₜ=1.651 min in 4 min chromatography, purity 98.1%, MS ESI calcd. for 477.19 [M+H]+ 478.19, found 478.2.

### Example 26. Synthesis of 2-(1-(5-(2-(2-aminopyridin-3-yl)-5-phenyl-3H-imidazo[4,5-b]pyridin-3-yl)pyridin-2-yl)pyrrolidin-3-yl)propanoic acid (Compound I-26) and 2-[1-[5-[2-(2-amino-3-pyridyl)-5-phenyl-imidazo[4,5-b]pyridin-3-yl]-2-pyridyl]pyrrolidin-3-yl]-2-methyl-propanoic acid (Compound I-39)

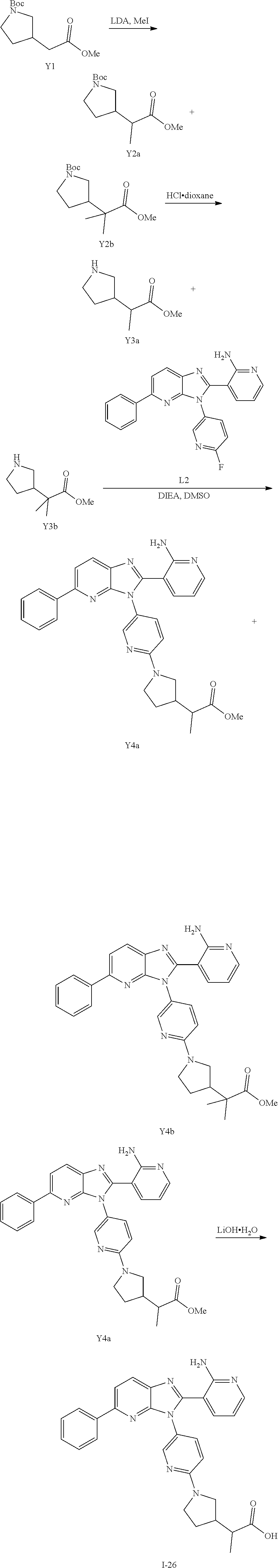

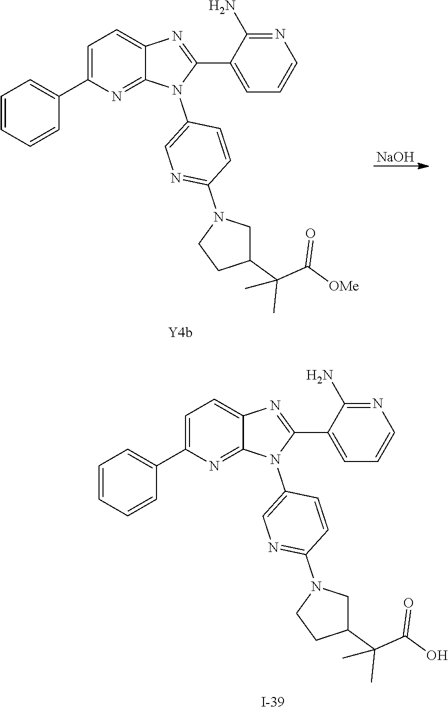

Step 1. To a stirred solution of tert-butyl 3-(2-methoxy-2-oxo-ethyl)pyrrolidine-1-carboxylate Y1 (400 mg, 1.64 mmol) in TH-F (5.00 mL), LDA (2 M in THF, 3.00 mL) was added slowly at −78° C., and the mixture was stirred at −78° C. under N₂ for 1 hr. MeI (0.510 mL, 8.22 mmol) was then added to the reaction mixture and the resulting mixture was stirred at 20° C. under N₂ for 1 hr. The reaction mixture was quenched with water (10 mL) and extracted with EtOAc (15 mL×2). The combined organic layers were washed with brine (20 mL), dried over anhydrous sodium sulfate, filtered and the filtrate was concentrated to afford a residue. The crude product was purified by flash chromatography on silica gel (0-100% of EtOAc in PE) to afford a mixture of tert-butyl 3-(2-methoxy-1,1-dimethyl-2-oxo-ethyl)pyrrolidine-1-carboxylate Y2b and tert-butyl 3-(2-methoxy-1-methyl-2-oxo-ethyl)pyrrolidine-1-carboxylate Y2a (260 mg, 61.5% yield) as a yellow oil.

Step 2. To a solution of tert-butyl 3-(2-methoxy-1,1-dimethyl-2-oxo-ethyl)pyrrolidine-1-carboxylate Y2b and tert-butyl 3-(2-methoxy-1-methyl-2-oxo-ethyl)pyrrolidine-1-carboxylate Y2a (260 mg, mixture) in 1,4-dioxane (1.00 mL), HCl/dioxane (3.00 mL) was added and the mixture was stirred at 25° C. for 16 hrs. The mixture was concentrated to give a mixture of methyl 2-methyl-2-pyrrolidin-3-yl-propanoate Y3b and methyl 2-pyrrolidin-3-ylpropanoate Y3a (160 mg, crude) as a yellow solid, which was used directly in the next step.

Step 3. To a stirred solution of 3-[3-(6-fluoro-3-pyridyl)-5-phenyl-imidazo[4,5-b]pyridin-2-yl]pyridin-2-amine L2 (150 mg, 0.390 mmol), the mixture of methyl 2-methyl-2-pyrrolidin-3-yl-propanoate Y3b and methyl 2-pyrrolidin-3-ylpropanoate Y3a (67.0 mg, 0.390 mmol) in DMSO (2.00 mL), DIEA (0.360 mL, 1.96 mmol) was added and the reaction mixture was stirred at 100° C. under N₂ for 16 hrs. To the mixture water (10 mL) was added and the mixture was extracted with EtOAc (20 mL×3). The organic phase was washed with brine (20 mL×3), dried with Na₂SO₄, filtered, and concentrated. The residue was purified by prep-HPLC (Xtimate C18 150*40 mm*5 μm, water (HCl)-ACN) to give methyl 2-[1-[5-[2-(2-amino-3-pyridyl)-5-phenyl-imidazo[4,5-b]pyridin-3-yl]-2-pyridyl]pyrrolidin-3-yl]propanoate Y4a (80.0 mg, 39.3% yield) as a yellow solid and methyl 2-[1-[5-[2-(2-amino-3-pyridyl)-5-phenyl-imidazo[4,5-b]pyridin-3-yl]-2-pyridyl]pyrrolidin-3-yl]-2-methyl-propanoate Y4b (70.0 mg, 33.4% yield) as a yellow solid. LCMS Rₜ=0.49 min in 1 min chromatography, Xtimate C18 2.1*30 mm,3 um, purity 93.7%, MS ESI calcd. for 519.24 [M+H]⁺ 520.24, found 520.3. LCMS Rₜ=0.51 min in 1 min chromatography, Xtimate C18 2.1*30 mm, 3 μm, purity 94.8%, MS ESI calcd. for 533.25 [M+H]⁺ 534.25, found 534.3.

Step 4a. To a solution of methyl 2-[1-[5-[2-(2-amino-3-pyridyl)-5-phenyl-imidazo[4,5-b]pyridin-3-yl]-2-pyridyl]pyrrolidin-3-yl]propanoate Y4a (50.0 mg, 0.0960 mmol) in THF (1.00 mL) and water (1.0.00 mL), LiOH·H₂O (12.0 mg, 0.290 mmol) was added and the mixture was stirred at 25° C. for 2 hrs. To the mixture LiOH·H₂O (12.0 mg, 0.290 mmol) was added and the mixture was stirred at 25° C. for 16 hrs. The mixture was concentrated and the resulting residue was purified by prep-HPLC (Xtimate C18 150*40 mm*5 μm, water (HCl)-ACN) to give 2-(1-(5-(2-(2-aminopyridin-3-yl)-5-phenyl-3H-imidazo[4,5-b]pyridin-3-yl)pyridin-2-yl)pyrrolidin-3-yl) propanoic acid I-26 (12.1 mg, 24.7% yield) as a yellow solid. ¹H NMR (MeOD 400 MHz) δ_{H}=8.45-8.38 (m, 1H), 8.35 (d, J=8.4 Hz, 1H), 8.21-8.05 (m, 6H), 7.53-7.42 (m, 3H), 7.39-7.29 (m, 1H), 7.03-6.96 (m, 1H), 4.03 (dd, J=7.6, 10.8 Hz, 1H), 3.95-3.83 (m, 1H), 3.78-3.66 (m, 1H), 3.47 (t, J=10.0 Hz, 1H), 2.80-2.66 (m, 1H), 2.65-2.52 (m, 1H), 2.43 (dd, J=5.2, 11.2 Hz, 1H), 2.07-1.87 (m, 1H), 1.35 (dd, J=7.2, 8.8 Hz, 3H). HPLC Rₜ=8.183 min in 15 min chromatography, 0-60AB, purity 99.4%. LCMS Rₜ=2.347 min in 4 min chromatography, Xtimate C18 2.1*30 mm, 3 μm, purity 100%, MS ESI calcd. for 505.22 [M+H]⁺ 506.22, found 506.1.

Step 4b. To a solution of methyl 2-[1-[5-[2-(2-amino-3-pyridyl)-5-phenyl-imidazo[4,5-b]pyridin-3-yl]-2-pyridyl]pyrrolidin-3-yl]-2-methyl-propanoate Y4b (30.0 mg, 0.0562 mmol) in THF (0.500 mL), aq. NaOH (4 M) was added and the mixture was stirred at 40° C. for 16 hrs. To the mixture, NaOH (22.0 mg, 0.562 mmol) and MeOH (1.00 mL) were added, and the mixture was stirred at 50° C. for 16 hrs. The mixture was concentrated, the resulting residue was dissolved with DMSO (2 mL), the solution adjusted to pH ~5, and the mixture was purified by prep-HPLC (Xtimate C18 150*40 mm*5 μm, water (HCl)-ACN) to give 2-[1-[5-[2-(2-amino-3-pyridyl)-5-phenyl-imidazo[4,5-b]pyridin-3-yl]-2-pyridyl]pyrrolidin-3-yl]-2-methyl-propanoic acid I-39 (16.7 mg, 56.4% yield) as a yellow solid. ¹H NMR (DMSO-d₆, 400 MHz) δ_{H}=8.33 (d, J=8.4 Hz, 1H), 8.24 (s, 1H), 8.05 (br d, J=8.0 Hz, 5H), 7.85 (br d, J=9.2 Hz, 1H), 7.53-7.39 (m, 3H), 6.98-6.82 (m, 2H), 3.71-3.68 (m, 2H), 3.47-3.36 (m, 1H), 3.28 (t, J=10.4 Hz, 1H), 2.65-2.60 (m, 1H), 2.13-1.98 (m, 1H), 1.91-1.73 (m, 1H), 1.17 (d, J=4.0 Hz, 6H). HPLC Rₜ=8.549 min in 15 min chromatography, 0-60AB, purity 99.5%. LCMS Rₜ=2.446 min in 4 min chromatography, Xtimate C18 2.1*30 mm, 3 μm, purity 100%, MS ESI calcd. for 519.24 [M+H]+ 520.24, found 520.1.

### Example 27. Synthesis of 1-(5-(2-(2-aminopyridin-3-yl)-5-phenyl-3H-imidazo[4,5-b]pyridin-3-yl)pyridin-2-yl)piperidine-4-carboxylic acid (Compound I-27)

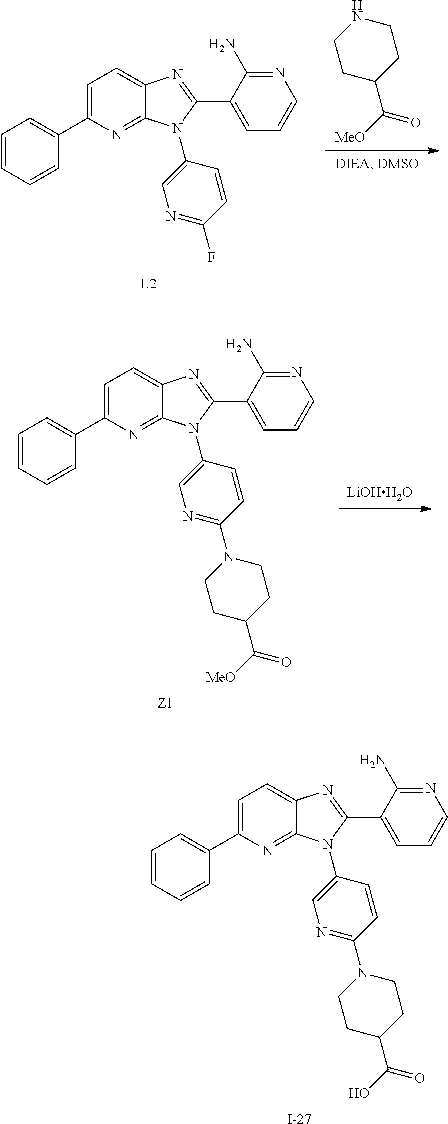

Step 1. To a stirred solution of 3-[3-(6-fluoro-3-pyridyl)-5-phenyl-imidazo[4,5-b]pyridin-2-yl]pyridin-2-amine L2 (50.0 mg, 0.130 mmol) and methyl piperidine-4-carboxylate (100 mg, 0.690 mmol) in DMSO (1.00 mL), DIEA (0.700 mL, 0.650 mmol) was added and the reaction mixture was stirred at 100° C. under N₂ for 10 hrs. The reaction mixture was diluted with EtOAc (10 mL), washed with water (15 mL) and brine (15 mL×2), dried over anhydrous sodium sulfate, filtered and the filtrate was concentrated to afford methyl 1-[5-[2-(2-amino-3-pyridyl)-5-phenyl-imidazo[4,5-b]pyridin-3-yl]-2-pyridyl]piperidine-4-carboxylate Z1 (50.0 mg, 75.6% yield) as a yellow oil. LCMS Rₜ=0.48 min in 1.0 min chromatography, purity 96.0%, MS ESI calcd. for 505.22 [M+H]⁺ 506.22, found 506.3.

Step 2. To a stirred solution of methyl 1-[5-[2-(2-amino-3-pyridyl)-5-phenyl-imidazo[4,5-b]pyridin-3-yl]-2-pyridyl]piperidine-4-carboxylate Z1 (50.0 mg, 0.100 mmol) in THF (1.00 mL) and water (1.00 mL), LiOH·H₂O (7.90 mg, 0.190 mmol) was added and the reaction mixture was stirred at 25° C. under N₂ for 3 hrs. The reaction mixture was adjusted to pH ~5 with 1N HCO and concentrated to afford a residue. The crude product was purified by prep-HPLC (column: Welch Xtimate C18 150×30 mm×5 μm, method: water (FA)-ACN, begin B: 21, end B: 51) to afford 1-[5-[2-(2-amino-3-pyridyl)-5-phenyl-imidazo[4,5-b]pyridin-3-yl]-2-pyridyl]piperidine-4-carboxylic acid I-27 (21.0 mg, 43.1% yield) as a light yellow solid. ¹H NMR (400 MHz, DMSO-d₆) δ=12.27 (s, 1H), 8.25 (d, J=8.4 Hz, 1H), 8.19 (d, J=2.4 Hz, 1H), 8.09-7.93 (m, 4H), 7.64 (dd, J=2.8, 9.2 Hz, 1H), 7.51-7.44 (m, 2H), 7.44-7.35 (m, 2H), 6.98 (d, J=9.2 Hz, 1H), 6.89 (s, 2H), 6.52 (dd, J=4.8, 7.6 Hz, 1H), 4.26 (d, J=13.2 Hz, 2H), 3.01 (t, J=11.2 Hz, 2H), 2.57-2.52 (m, 1H), 1.99-1.85 (m, 2H), 1.70-1.46 (m, 2H). HPLC Rₜ=3.194 min in 8 min chromatography, purity 99.7%. LCMS Rₜ=1.829 min in 4 min chromatography, purity 99.7%, MS ESI calcd. for 491.21 [M+H]⁺ 492.21, found 492.1.

### Example 28. Synthesis of 1-(5-(2-(2-aminopyridin-3-yl)-5-phenyl-3H-imidazo[4,5-b]pyridin-3-yl)pyridin-2-yl)piperidine-3-carboxylic acid (Compound I-28)

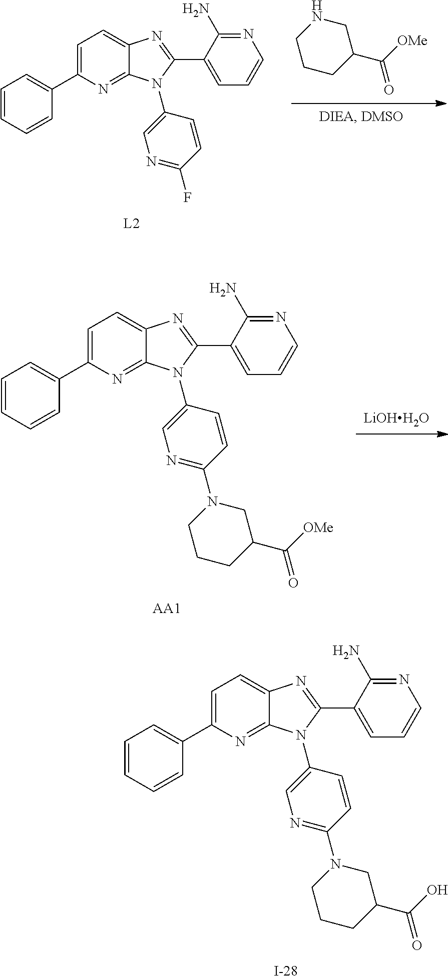

Step 1. To a solution of 3-[3-(6-fluoro-3-pyridyl)-5-phenyl-imidazo[4,5-b]pyridin-2-yl]pyridin-2-amine L2 (70.0 mg, 0.183 mmol) in DMSO (1.50 mL), 1-(3-piperidyl)ethanone (93.0 mg, 0.732 mmol) and DIEA (0.810 mL, 4.39 mmol) were added at 25° C. under N₂, and the mixture was stirred at 120° C. for 16 hrs. Water (5.00 mL) was added and the mixture was extracted with EtOAc (5.00 mL×3). The organic phase was washed with brine (5.00 mL×3), dried over Na₂SO₄, filtered and concentrated under reduced pressure. The crude was purified by flash chromatography eluting with EtOAc in PE (0% to 100%) to give methyl 1-[5-[2-(2-amino-3-pyridyl)-5-phenyl-imidazo[4,5-b]pyridin-3-yl]-2-pyridyl]piperidine-3-carboxylate AA1 (60.0 mg, 64.8% yield) as a red oil. ¹H NMR (400 MHz, DMSO-d₆) δ=8.25 (d, J=8.4 Hz, 1H), 8.19 (d, J=2.4 Hz, 1H), 8.06-8.00 (m, 3H), 7.97 (d, J=8.4 Hz, 1H), 7.64 (dd, J=2.8, 9.2 Hz, 1H), 7.50-7.44 (m, 2H), 7.42-7.36 (m, 2H), 6.99 (d, J=9.2 Hz, 1H), 6.85 (s, 2H), 6.51 (dd, J=4.8, 7.6 Hz, 1H), 3.62 (s, 3H), 3.24-3.07 (m, 2H), 2.63-2.55 (m, 2H), 2.03-1.98 (m, 2H), 1.81-1.65 (m, 2H), 1.57-1.49 (m, 1H).

Step 2. To a solution of methyl 1-[5-[2-(2-amino-3-pyridyl)-5-phenyl-imidazo[4,5-b]pyridin-3-yl]-2-pyridyl]piperidine-3-carboxylate AA1 (60.0 mg, 0.120 mmol) in THE (1.00 mL) and water (1.00 mL), LiOH·H₂O (12.4 mg, 0.296 mmol) was added at 25° C. and the mixture stirred for 16 hrs. The crude material was purified by prep-HPLC (Column C18 150×30 mm water (HCl)-ACN) to give 1-(5-(2-(2-aminopyridin-3-yl)-5-phenyl-3H-imidazo[4,5-b]pyridin-3-yl)pyridin-2-yl)piperidine-3-carboxylic acid 1-28 (15.0 mg, 25.4% yield) as a yellow solid. ¹H NMR (400 MHz, DMSO-d₆) δ=8.35 (d, J=8.8 Hz, 1H), 8.27 (d, J=2.4 Hz, 1H), 8.14 (dd, J=1.6, 6.4 Hz, 1H), 8.08-8.02 (m, 4H), 7.77 (dd, J=2.4, 9.2 Hz, 1H), 7.53-7.47 (m, 2H), 7.46-7.40 (m, 1H), 7.09 (d, J=9.2 Hz, 1H), 6.98 (dd, J=6.4, 7.6 Hz, 1H), 4.39 (dd, J=3.2, 12.8 Hz, 1H), 4.08 (d, J=12.8 Hz, 1H), 3.24-3.05 (m, 3H), 2.04-1.96 (m, 1H), 1.76-1.62 (m, 2H), 1.58-1.47 (m, 1H). HPLC Rₜ=3.319 min in 8.0 min chromatography, 10-80AB_8 min.1cm, purity 96.1%. LCMS Rₜ=1.871 min in 4.0 min chromatography, purity 97.3%. MS ESI calcd. for 491.21 [M+H]⁺ 492.21, found 492.1.

### Example 29. Synthesis of 1-(4-(2-(2-aminopyridin-3-yl)-5-phenyl-3H-imidazo[4,5-b]pyridin-3-yl)benzyl)piperidine-4-carboxylic acid (Compound I-29)

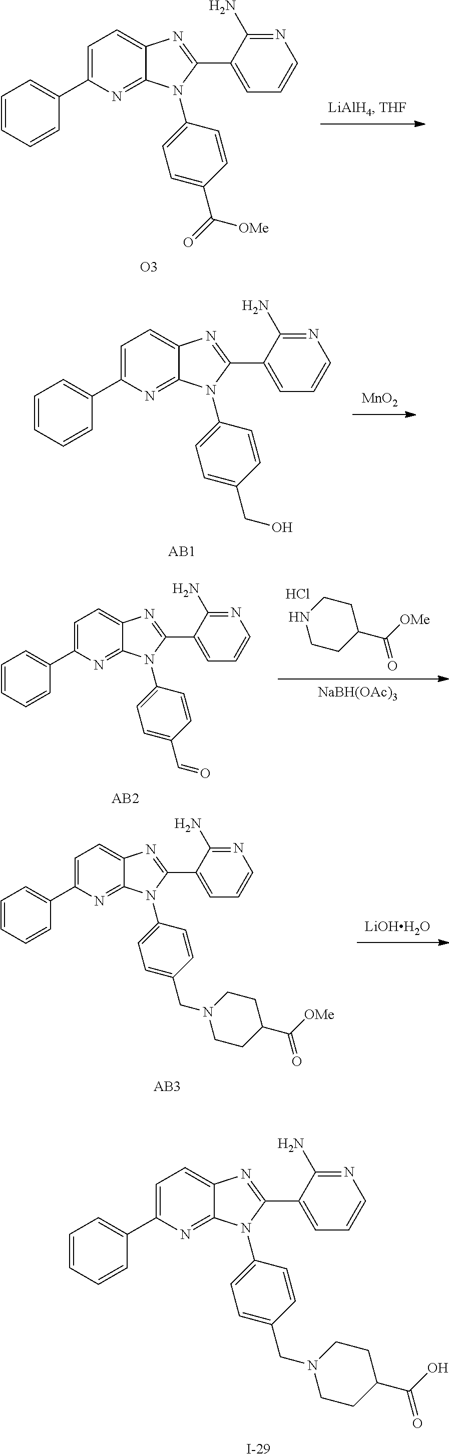

Step 1. To a solution of methyl 4-[2-(2-amino-3-pyridyl)-5-phenyl-imidazo[4,5-b]pyridin-3-yl]benzoate O3 (500 mg, 1.19 mmol) in THF (10.0 mL), LiAlH₄ (0.180 g, 4.75 mmol) was added at 0° C., and the reaction mixture was stirred at 25° C. for 2 hrs. The reaction was quenched with saturated aqueous NH₄Cl (20 mL), dropwise, and the resulting mixture was extracted with EtOAc (20 mL×3). The combined organic phase was dried over anhydrous Na₂SO₄, filtered and concentrated to give [4-[2-(2-amino-3-pyridyl)-5-phenyl-imidazo[4,5-b]pyridin-3-yl]phenyl]methanol ABI (564 mg, crude) as a yellow solid, which was used for the next step without further purification. LCMS Rₜ=0.699 min in 1.5 min chromatography, purity 6.36%, MS ESI calcd. for 393.16 [M+H]⁺ 394.16, found 394.1.

Step 2. To a solution of [4-[2-(2-amino-3-pyridyl)-5-phenyl-imidazo[4,5-b]pyridin-3-yl]phenyl]methanol AB1 (500 mg, 1.27 mmol) in DCE (20.0 mL), MnO₂ (5.00 g, 57.5 mmol) was added at 25° C., and the reaction mixture was stirred at 60° C. for 12 hrs. The mixture was filtered via a celite pad and the filter cake was washed with DCM/EtOAc (1/1 (1 L)). The organic phase was concentrated to give crude product. The residue was purified by flash silica gel chromatography (ISCO®; 4 g SepaFlash® Silica Flash Column, Eluent with 47% of Ethyl acetate in Petroleum ether gradient @35 mL/min) to give 4-[2-(2-amino-3-pyridyl)-5-phenyl-imidazo[4,5-b]pyridin-3-yl]benzaldehyde AB2 (150 mg, 46.2% yield) as a yellow solid. LCMS Rₜ=1.249 min in 2.5 min chromatography, purity 36.4%, MS ESI calcd. for 391.14 [M+H]⁺ 392.14, found 392.1.

Step 3. To a solution of 4-[2-(2-amino-3-pyridyl)-5-phenyl-imidazo[4,5-b]pyridin-3-yl]benzaldehyde AB2 (50.0 mg, 0.120 mmol) in DCM (2.00 mL), methyl piperidine-4-carboxylate (27.0 mg, 0.190 mmol) was added and the reaction mixture was stirred at 25° C. for 2 hrs. Sodium triacetoxyborohydride (81.0 mg, 0.380 mmol) was added and the resulting mixture was stirred at 25° C. for 3 hrs. The mixture was diluted with H₂O (10 mL), extracted with DCM (10 mL×3), and washed with brine (10 mL). The organic layer was dried over Na₂SO₄, filtered, and concentrated. The residue was purified by flash silica gel chromatography (ISCO®; 4 g SepaFlash® Silica Flash Column, Eluent with 70% of Ethyl acetate in Petroleum ether gradient @35 mL/min) to give methyl 1-[[4-[2-(2-amino-3-pyridyl)-5-phenyl-imidazo[4,5-b]pyridin-3-yl]phenyl]methyl]piperidine-4-carboxylate AB3 (65.0 mg, crude) as a yellow solid. LCMS Rₜ=0.977 min in 2.5 min chromatography, purity 16.2%, MS ESI calcd. for 518.24 [M+H]⁺ 519.24, found 519.2.

Step 4. To a solution of methyl 1-[[4-[2-(2-amino-3-pyridyl)-5-phenyl-imidazo[4,5-b]pyridin-3-yl]phenyl]methyl]piperidine-4-carboxylate AB3 (65.0 mg, 0.120 mmol) in THF (2.00 mL), LiOH·H₂O (21.0 mg, 0.500 mmol) and water (1.00 mL) were added and the mixture was stirred at 25° C. for 2 hrs. The reaction mixture was concentrated, and water (10 mL) was subsequently added and the whole extracted with PE (10 mL×3). The pH of the aqueous phase was adjusted to pH ~5 with HCl (2 M) and the mixture extracted with DCM (10 mL×3). The combined organic phase was dried over anhydrous Na₂SO₄, filtered and concentrated. The crude product was purified by prep-HPLC (column: Welch Xtimate C18 40*200 mm*7 μm; mobile phase: [water (FA)-ACN]; B %: 0%-32%, 30 min) to give 1-(4-(2-(2-aminopyridin-3-yl)-5-phenyl-3H-imidazo[4,5-b]pyridin-3-yl)benzyl)piperidine-4-carboxylic acid I-29 (24.6 mg, 38.9% yield) as a white solid. ¹H NMR (DMSO-d₆, 400 MHz) δ 8.27 (d, J=8.4 Hz, 1H), 8.07-7.96 (m, 4H), 7.51-7.37 (m, 7H), 7.16 (dd, J=7.6, 1.2 Hz, 1H), 7.01 (s, 2H), 6.38 (dd, J=7.6, 4.8 Hz, 1H), 3.55 (s, 2H), 2.82-2.75 (m, 2H), 2.28-2.18 (m, 1H), 2.11-2.00 (m, 2H), 1.87-1.77 (m, 2H), 1.65-1.52 (m, 2H). HPLC Rₜ=3.131 min in 8 min chromatography, purity 96.6%. LCMS Rₜ=1.823 min in 4 min chromatography, purity 100%, MS ESI calcd. for 504.23 [M+H]⁺ 505.23, found 505.2.

### Example 30. Synthesis of 1-(4-(2-(2-aminopyridin-3-yl)-5-phenyl-3H-imidazo[4,5-b]pyridin-3-yl)-2-fluorobenzyl)piperidine-4-carboxylic acid (Compound I-30)

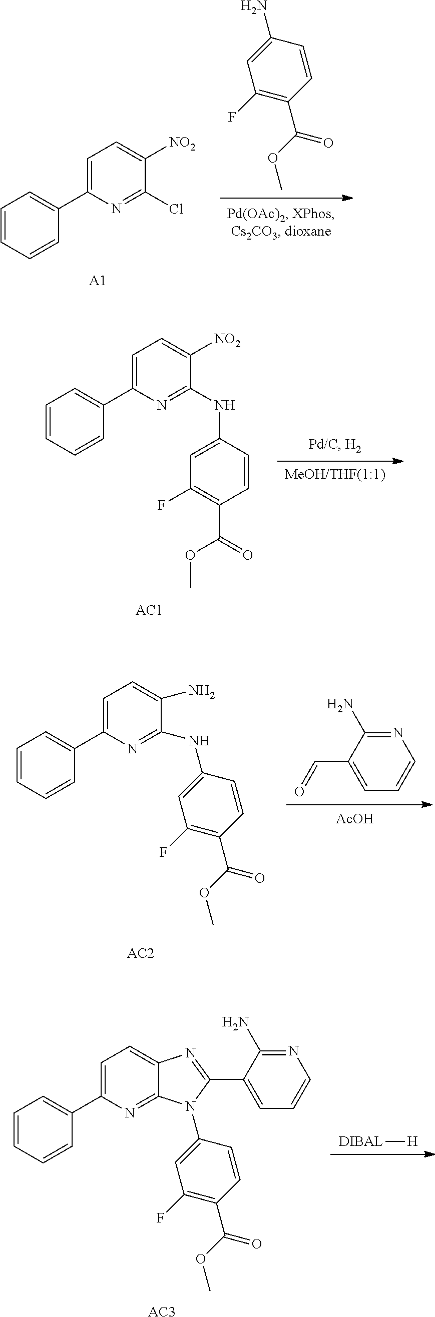

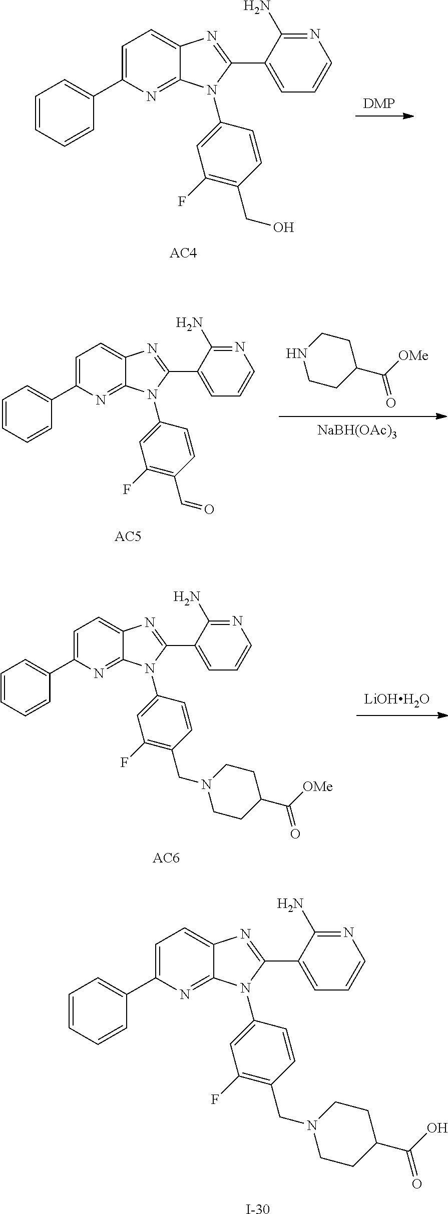

Step 1. To a solution of 2-chloro-3-nitro-6-phenylpyridine A1 (6.00 g, 25.6 mmol) and methyl 4-amino-2-fluorobenzoate (4.33 g, 25.6 mmol) in 1,4-dioxane (100 mL), XPhos (610 mg, 1.28 mmol), Pd(OAc)₂ (1.01 g, 1.28 mmol) and Cs₂CO₃ (24.9 g, 76.7 mmol) were added at 25° C. and the mixture was stirred at 110° C. for 5 hrs. The reaction mixture was filtered, and the filter cake was washed with DCM (300 mL). The filtrate was concentrated, the residue was dissolved in water (100 mL) and the resulting suspension was extracted with DCM (200 mL×3). The combined organic phase was washed with brine (100 mL), dried over anhydrous Na₂SO₄, filtered and concentrated. The residue was purified by Combi-Flash column (EtOAc in PE=0%-15%) to afford methyl 2-fluoro-4-((3-nitro-6-phenylpyridin-2-yl)amino)benzoate AC1 (8.64 g, 74.4% yield) as a yellow solid. ¹H NMR (DMSO-d₆, 400 MHz) δ_{H}=10.26 (s, 1H), 8.63 (d, J=8.8 Hz, 1H), 8.11 (dd, J=2.8, 6.8 Hz, 2H), 7.98-7.85 (m, 2H), 7.77-7.65 (m, 2H), 7.61-7.51 (m, 3H), 3.85 (s, 3H).

Step 2. To a solution of methyl 2-fluoro-4-((3-nitro-6-phenylpyridin-2-yl)amino)benzoate AC1 (8.30 g, 22.6 mmol) in THF (50.0 mL) and MeOH (50.0 mL), wet Pd/C (4.00 g, 10% purity) was added under Ar atmosphere. The suspension was degassed and purged with H₂ (×3), and the reaction mixture was stirred under H₂ (15 psi) at 25° C. for 12 hrs. The reaction mixture was filtered and the filter cake was washed with MeOH/DCM (1/1, 1 L). The resulting suspension was concentrated directly to afford methyl 4-((3-amino-6-phenylpyridin-2-yl)amino)-2-fluorobenzoate AC2 (7.75 g, crude) as a yellow solid ¹H NMR (DMSO-d₆, 400 MHz) δ_{H}=8.71 (s, 1H), 7.96-7.86 (m, 3H), 7.82 (t, J=8.8 Hz, 1H), 7.50-7.37 (m, 4H), 7.32-7.23 (m, 1H), 7.09 (d, J=8.0 Hz, 1H), 5.44 (s, 2H), 3.80 (s, 31H).

Step 3. To a solution of methyl 4-((3-amino-6-phenylpyridin-2-yl)amino)-2-fluorobenzoate AC2 (7.75 g, 23.0 mmol) in acetic acid (50.0 mL), 2-aminonicotinaldehyde (3.37 g, 27.6 mmol) was added at 25° C., and the mixture was stirred at 100° C. for 2 hrs. EtOAc (50.0 mL) was added, and the resulting mixture was stirred at 25° C. for 1 hr. The mixture was filtered and the filter cake was washed with EtOAc (50.0 mL). The filtrate was concentrated directly. The filter cake was dissolved in DCM (100 mL). The resulting suspension was washed with saturated aqueous NaHCO₃ (30 mL×3). The combined organic phase was washed with brine (50.0 mL), dried over anhydrous Na₂SO₄, filtered and concentrated. The filtrate was purified by Combi-Flash column (EtOAc in PE=0%-58%) to afford methyl 4-(2-(2-aminopyridin-3-yl)-5-phenyl-3H-imidazo[4,5-b]pyridin-3-yl)-2-fluorobenzoate AC3 (500 mg, 4.95% yield) as a yellow solid. ¹H NMR (DMSO-d₆, 400 MHz) δ_{H}=8.29 (d, J=8.4 Hz, 1H), 8.13-7.95 (m, 5H), 7.65 (d, J=11.6 Hz, 1H), 7.53-7.38 (m, 4H), 7.34 (d, J=7.2 Hz, 1H), 6.78 (s, 2H), 6.52 (dd, J=4.8, 6.8 Hz, 1H), 3.89 (s, 3H).

Step 4. To a solution of methyl 4-(2-(2-aminopyridin-3-yl)-5-phenyl-3H-imidazo[4,5-b]pyridin-3-yl)-2-fluorobenzoate AC3 (500 mg, 1.14 mmol) in DCM (8.00 mL), DIBAL-H (5.70 mL, 5.69 mmol) was added at 0° C. under N₂ atmosphere, and the mixture was stirred at 25° C. for 4 hrs. The reaction was quenched with saturated aqueous potassium sodium tartrate (15.0 mL), dropwise, at 0° C. The resulting mixture was extracted with DCM (15 mL, 3). The combined organic phase was washed with brine (8.00 mL), dried over anhydrous Na₂SO₄, filtered and concentrated. The residue was purified by Combi-Flash column (EtOAc in PE=0%-70/6) to afford (4-(2-(2-aminopyridin-3-yl)-5-phenyl-3H-imidazo[4,5-b]pyridin-3-yl)-2-fluorophenyl)methanol AC4 (255 mg, 54.5% yield) as a yellow solid. ¹H NMR (DMSO-4.400 MHz) δ_{H}=8.27 (d, J=8.4 Hz, 1H), 8.07-7.98 (m, 4H), 7.61 (t, J=8.0 Hz, 1H), 7.50-7.44 (m, 3H), 7.41 (d, J=7.2 Hz, 1H), 7.30 (d, J=8.0 Hz, 2H), 6.88 (s, 2H), 648 (dd, J=4.8, 7.6 Hz, 1H), 4.63 (d, J=6.0 Hz, 2H).

Step 5. To a solution of (4-(2-(2-aminopyridin-3-yl)-5-phenyl-3H-imidazo[4,5-b]pyridin-3-yl)-2-fluorophenyl)methanol AC4 (255 mg, 0.620 mmol) in DCM (4.00 mL), DMP (315 mg, 0.740 mmol) was added at 0° C. and the mixture stirred at 25° C. for 1 hr. The reaction was quenched with saturated aqueous NaHCO₃/Na₂S₂O₃ (6 mL, 1/1), dropwise, and the mixture was extracted with DCM (8 mL×3). The combined organic phase was washed with brine (8 mL), dried over anhydrous Na₂SO₄, filtered and concentrated. The residue was purified by Combi-Flash column (EtOAc in PE=0%-50%) to afford 4-(2-(2-aminopyridin-3-yl)-5-phenyl-3H-imidazo[4,5-b]pyridin-3-yl)-2-fluorobenzaldehyde ACS (143 mg, 56.4% yield) as a yellow solid. ¹H NMR (DMSO-d₆, 400 MHz) δ=10.25 (s, 1H), 8.31 (d, J=8.4 Hz, 1H), 8.09 (d, J=1.6 Hz, 1H), 8.08-8.05 (m, 2H), 8.04-8.01 (m, 1H), 8.01-7.98 (m, 1H), 7.71 (dd, J=1.6, 11.2 Hz, 1H), 7.53-7.46 (m, 3H), 7.45-7.40 (m, 1H), 7.36 (dd, J=2.0, 7.6 Hz, 1H), 6.77 (s, 2H), 6.52 (dd, J=4.8, 7.6 Hz, 1H).

Step 6. A mixture of 4-(2-(2-aminopyridin-3-yl)-5-phenyl-3H-imidazo[4,5-b]pyridin-3-yl)-2-fluorobenzaldehyde AC5 (110 mg, 0.270 mmol) and methyl piperidine-4-carboxylate (77.0 mg, 0.540 mmol) in DCM (2.00 mL) was degassed and purged with N₂ (×3), and the mixture stirred at 25° C. for 3 hrs under N₂ atmosphere. NaBH(OAc)₃ (171 mg, 0.810 mmol) was added into the mixture at 25° C. under N₂ atmosphere, and the resulting mixture was stirred for 2 hrs. The reaction was quenched with water (3.00 mL), drop-wise, and the resulting mixture was extracted with DCM (8 mL×3). The combined organic phase was washed with brine (5 mL), dried over anhydrous Na₂SO₄, filtered and concentrated. The residue was purified by Combi-Flash column (EtOAc in PE=0%-65%) to afford methyl 1-(4-(2-(2-aminopyridin-3-yl)-5-phenyl-3H-imidazo[4,5-b]pyridin-3-yl)-2-fluorobenzyl)piperidine-4-carboxylate AC6 (120 mg, 83.2% yield) as a yellow solid. ¹H NMR (DMSO-d₆, 400 MHz,) δ=8.28 (d, 0.1=8.4 Hz, 1H), 8.08-7.97 (m, 4H), 7.55 (t, J=8.0 Hz, 1H), 7.48 (t, J=7.2 Hz, 3H), 7.44-7.37 (m, 1H), 7.33-7.22 (m, 2H), 6.92 (s, 2H), 6.52-6.39 (m, 1H), 3.60 (s, 5H), 2.80 (d, J=11.2 Hz, 2H), 2.33-2.29 (m, 1H), 2.09 (t, J=10.8 Hz, 2H), 1.87-1.78 (m, 2H), 1.67-1.52 (m, 2H).

Step 7. A solution of methyl 1-(4-(2-(2-aminopyridin-3-yl)-5-phenyl-3H-imidazo[4,5-b]pyridin-3-yl)-2-fluorobenzyl)piperidine-4-carboxylate AC6 (120 mg, 0.220 mmol) and LiOH·H₂O (28.0 mg, 0.670 mmol) in water (2.00 mL) and THF (2.00 mL) was stirred at 25° C. for 1 hr. The mixture was filtered directly and was purified by prep-HPLC (column: Xtimate C18 150*40 mm*7 μm; mobile phase: [water(NH₄HCO₃)-ACN]; B %: 4%-44%, 25 min) to afford 1-(4-(2-(2-aminopyridin-3-yl)-5-phenyl-3H-imidazo[4,5-b]pyridin-3-yl)-2-fluorobenzyl)piperidine-4-carboxylic acid 1-30 (61.0 mg, 52.2% yield) as a yellow solid. ¹H NMR (DMSO-d₆, 400 MHz) δ=8.28 (d, J=8.4 Hz, 1H), 8.09-7.94 (m, 4H), 7.55 (t, J=8.0 Hz, 1H), 7.51-7.44 (m, 3H), 7.44-7.37 (m, 1H), 7.31-7.21 (m, 2H), 6.91 (s, 2H), 6.45 (dd, J=4.8, 7.8 Hz, 1H), 3.59 (s, 2H), 2.79 (d, J=11.2 Hz, 2H), 2.26-2.15 (m, 1H), 2.15-2.01 (m, 2H), 1.81 (d, J=10.4 Hz, 2H), 1.63-1.52 (m, 2H). HPLC Rₜ=1.673 min in 8 min chromatography, purity 100%. LCMS Rₜ=1.150 min in 4 min chromatography, purity 99.8%, MS ESI calcd. for 522.22 [M+H]⁺ 523.22, found 523.2.

### Example 31. Synthesis of 4-((4-(2-(2-aminopyridin-3-yl)-5-phenyl-3H-imidazo[4,5-b]pyridin-3-yl)benzyl)amino)cyclohexane-1-carboxylic acid (Compound I-31)

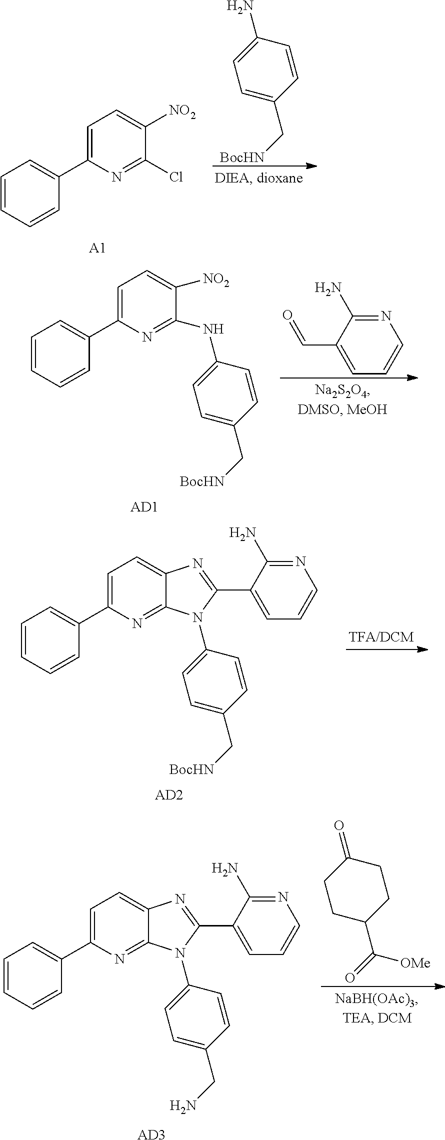

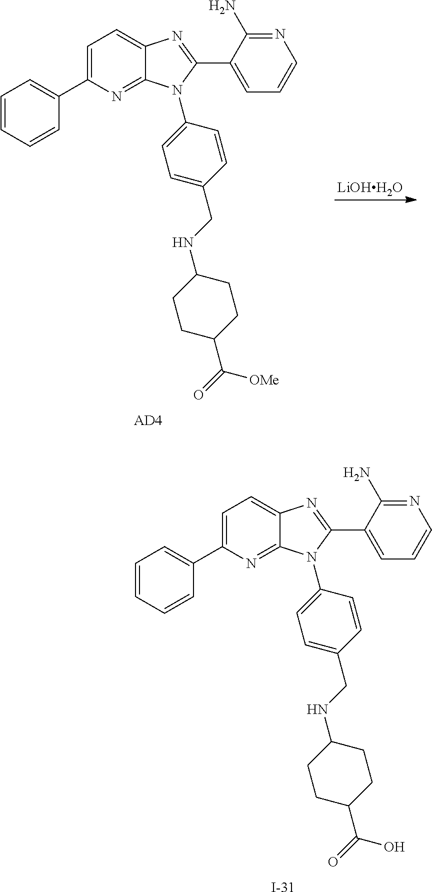

Step 1. To a solution of 2-chloro-3-nitro-6-phenylpyridine A1 (15.0 g, 63.9 mmol) and tert-butyl N-[(4-aminophenyl)methyl]carbamate (19.2 g, 86.3 mmol) in 1,4-dioxane (50.0 mL), DIEA (32.0 mL, 192 mmol) was added and the mixture stirred at 80° C. for 12 hrs. Water (20 mL) was added, and the resulting mixture was extracted with EtOAc (30.0 mL×3). The combined organic phase was washed with brine (30 mL×3), dried over anhydrous Na₂SO₄, filtered and concentrated. The residue was purified by flash silica gel chromatography (eluent with 0-30% of EtOAc in PE) to afford tert-butyl N-[[4-[(3-nitro-6-phenyl-2-pyridyl)amino]phenyl]methyl]carbamate AD1 (26.0 g, 96.7% yield) as a yellow solid. ¹H NMR (DMSO-d₆, 400 MHz) δ_{H}=10.09 (s, 1H), 8.60 (d, J=8.8 Hz, 1H), 8.11-8.07 (m, 2H), 7.69 (d, J=8.4 Hz, 2H), 7.58 (d, J=8.8 Hz, 1H), 7.55-7.50 (m, 3H), 7.29 (d, J=8.4 Hz, 2H), 4.15 (d, J=6.0 Hz, 2H), 1.41 (m, 9H).

Step 2. To a solution of tert-butyl N-[[4-[(3-nitro-6-phenyl-2-pyridyl)amino]phenyl]methyl]carbamate AD1 (30.0 g, 71.3 mmol) and 2-aminopyridine-3-carbaldehyde (11.8 g, 96.3 mmol) in DMSO (100 mL) and methanol (100 mL), Na₂S₂O₄ (37.3 g, 214 mmol) was added, under N₂, and the mixture was stirred at 25° C. for 12 hrs. The reaction mixture was concentrated, aq. LiCl (30 mL) was added to the residue and the resulting mixture was extracted with EtOAc (30 mL×3). The combined organic phase was washed with brine (30 mL), dried over anhydrous Na₂SO₄, filtered and concentrated. The residue was purified by flash silica gel chromatography (eluent with 0-5% of MeOH in DCM) to afford tert-butyl N-[[4-[2-(2-amino-3-pyridyl)-5-phenyl-imidazo[4,5-b]pyridin-3-yl]phenyl]methyl]carbamate AD2 (5.90 g, 15.9% yield) as a yellow solid. ¹H NMR (DMSO-d₆, 400 MHz) δ_{H}=8.26 (d, J=8.4 Hz, 1H), 8.04-7.97 (m, 4H), 7.47-7.39 (m, 7H), 7.22-7.18 (m, 1H), 6.99 (s, 2H), 6.44-6.37 (m, 1H), 4.28-4.20 (m, 2H), 1.41 (s, 9H).

Step 3. To a solution of tert-butyl N-[[4-[2-(2-amino-3-pyridyl)-5-phenyl-imidazo[4,5-b]pyridin-3-yl]phenyl]methyl]carbamate AD2 (100 mg, 0.20 mmol) in DCM (2.50 mL), TFA (0.50 mL) was added and the mixture was stirred at 25° C. for 12 hrs. The reaction mixture was concentrated directly and the resulting residue was purified by prep-HPLC (Column. Welch Xtimate C18 150*30 mm*5 μm; mobile phase: [water (0.225% FA)-ACN]; B %: 0%-26%; 36 min) to afford 3-[3-[4-(aminomethyl)phenyl]-5-phenyl-imidazo[4,5-b]pyridin-2-yl]pyridin-2-amine AD3 (19.1 mg, 24.0% yield) as a light yellow solid. ¹H NMR (MeOD, 400 MHz) δ_{H}=8.21 (d, J=8.4 Hz, 1H), 8.04-7.93 (m, 4H), 7.68-7.57 (m, 4H), 7.45-7.35 (m, 4H), 6.48 (dd, J1=5.2, 7.6 Hz, 1H), 4.22 (s, 2H). HPLC Rₜ=3.920 min in 8 min chromatography, purity 100%. LCMS Rₜ=2.005 min in 4 min chromatography, purity 99.5%, MS ESI calcd. for 392.17, [M+H]⁺ 393.17, found 393.4.

Step 4. To a solution of 3-[3-[4-(aminomethyl)phenyl]-5-phenyl-imidazo[4,5-b]pyridin-2-yl]pyridin-2-amine AD3 (186 mg, 0.470 mmol) in DCM (3.00 mL), TEA (2.00 mL), and then methyl 4-oxocyclohexanecarboxylate (89.0 mg, 0.570 mmol) and AcOH (0.10 mL) were added at 25° C. under N₂. After 2 hrs, NaBH(OAc)₃ (222 mg, 0.71 mmol) was added and the mixture was stirred at 25° C. for an additional 16 hrs under N₂. Water (20.0 mL) was added and the mixture was extracted with DCM (20.0 mL×3). The organic phase was washed with brine (20.0 mL×3), dried over Na₂SO₄, filtered and concentrated under reduced pressure. The crude was purified by flash chromatography eluting with DCM in MeOH (0%-10%) to give methyl 4-((4-(2-(2-aminopyridin-3-yl)-5-phenyl-3H-imidazo[4,5-b]pyridin-3-yl)benzyl)amino)cyclohexane-1-carboxylate AD4 (178 mg, 70.6% yield) as a yellow oil. ¹H NMR (400 MHz, DMSO-d₆) δ=8.27 (d, J=8.4 Hz, 1H), 8.04-7.98 (m, 4H), 7.55 (d, J=7.6 Hz, 2H), 7.49-7.43 (m, 4H), 7.42-7.37 (m, 1H), 7.22-7.16 (m, 1H), 7.00 (br s, 1H), 6.39 (dd, J=4.8, 7.6 Hz, 1H), 3.90 (d, 0.1=16.4 Hz, 2H), 3.64-3.57 (m, 3H), 2.81-2.58 (m, 1H), 2.36-2.20 (m, 1H), 1.97-1.87 (m, 2H), 1.57-1.42 (m, 3H), 1.41-1.29 (m, 1H), 1.25-1.15 (m, 2H)

Step 5. To a solution of methyl 4-((4-(2-(2-aminopyridin-3-yl)-5-phenyl-3H-imidazo[4,5-b]pyridin-3-yl)benzyl)amino)cyclohexane-1-carboxylate AD4 (168 mg, 0.320 mmol) in THF (2.00 mL) and water (2.00 mL), LiOH·H₂O (26.0 mg, 0.630 mmol) was added at 25° C., and the mixture was stirred for 16 hrs. The mixture was concentrated under reduced pressure. HCl was added and the pH was adjusted to pH 2~3. The crude material was purified by prep-HPLC (Column C18 150×40 mm water (HCl)-ACN) to give 4-((4-(2-(2-aminopyridin-3-yl)-5-phenyl-3H-imidazo[4,5-b]pyridin-3-yl)benzyl)amino)cyclohexane-1-carboxylic acid I-31 (37.0 mg, 22.2% yield) as a yellow solid. ¹H NMR (400 MHz, DMSO-d₆) δ=9.22 (br s, 1H), 8.61-8.40 (m, 1H), 8.37 (d, J=8.8 Hz, 1H), 8.15 (dd, J=1.6, 6.0 Hz, 1H), 8.07 (t, J=8.8 Hz, 3H), 7.92-7.87 (m, 1H), 7.82-7.76 (m, 2H), 7.65 (d, J=8.4 Hz, 2H), 7.51-7.46 (m, 2H), 7.45-7.40 (m, 1H), 6.89 (t, J=6.4 Hz, 1H), 4.24 (br s, 2H), 2.60-2.57 (m, 1H), 2.27-2.14 (m, 1H), 2.13-1.96 (m, 4H), 1.67-1.45 (m, 4H). HPLC Rₜ=2.902 min in 8.0 min chromatography, 10-80AB_8 min.1cm, purity 97.8%. LCMS Rₜ=1.539 min in 4.0 min chromatography, purity 96.6%, MS ESI calcd. for 518.24 [M+H]⁺ 519.24. found 519.1.

### Example 32. Synthesis of (1r,4r)-4-((5-(2-(2-aminopyridin-3-yl)-6-phenyl-1H-benzo[d]imidazol-1-yl)pyridin-2-yl)carbamoyl)cyclohexane-1-carboxylic acid (Compound I-32)

### Synthesis of methyl (1r,4r)-4-(chlorocarbonyl)cyclohexane-1-carboxylate (AE2)

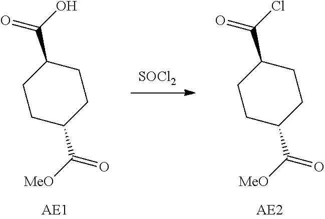 [SMILES: COC(=O)[C@H]1CC[C@H](C(=O)O)CC1 ; "SOCl2" ; COC(=O)[C@H]1CC[C@H](C(=O)Cl)CC1]

Step 1. To a solution of (1r,4r)-4-(methoxycarbonyl)cyclohexane-1-carboxylic acid AE1 (1.00 g, 5.37 mmol) in DCM (15.0 mL) and DMF (0.100 mL), SOCl₂ (1.20 mL, 16.1 mmol) was added at 0° C., and the mixture was stirred at 25° C. for 2 hrs. The reaction mixture was concentrated directly to give methyl (1r,4r)-4-(chlorocarbonyl)cyclohexane-1-carboxylate AE2 (1.01 g, 70.9% yield) as a white solid, which was used directly in the next step.

### Synthesis of (1r,4r)-4-((5-(2-(2-aminopyridin-3-yl)-6-phenyl-1H-benzo[d]imidazol-1-yl)pyridin-2-yl)carbamoyl)cyclohexane-1-carboxylic acid (Compound I-32)

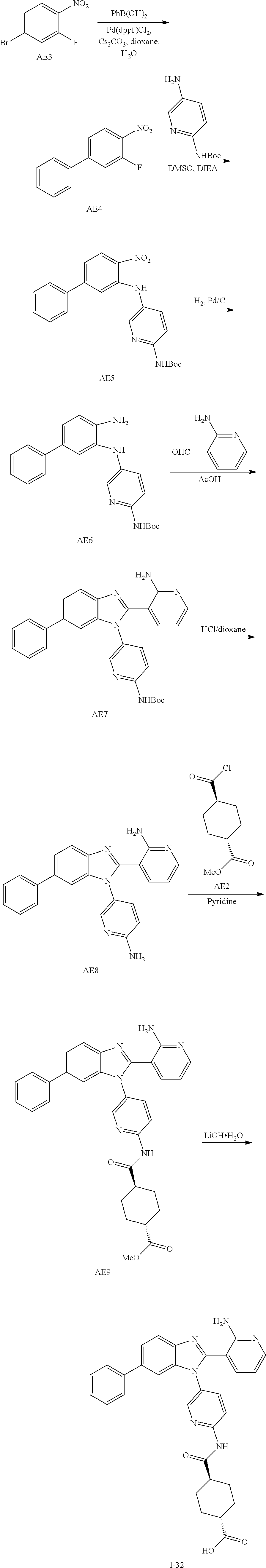

Step 1. To a solution of 4-bromo-2-fluoro-1-nitro-benzene AE3 (2.00 g, 9.09 mmol) and phenylboronic acid (1.44 g, 11.8 mmol) in 1,4-dioxane (30.0 mL) and water (5.00 mL), Cs₂CO₃ (8.89 g, 27.3 mmol) and Pd(dppf)Cl₂ (742 mg, 0.909 mmol) were added and the mixture was stirred at 85° C. for 12 hrs under N₂. The reaction mixture was filtered, and the filtrate was concentrated and purified by flash silica gel chromatography (eluent of 0%-1% EtOAc/PE) to give 3-fluoro-4-nitro-1,1′-biphenyl AE4 (2.19 g, 99.8% yield) as a yellow solid. ¹H NMR (400 MHz, CDCl₃) δ=8.21-8.11 (m, 1H), 7.65-7.58 (m, 2H), 7.56-7.46 (m, 5H).

Step 2. To a solution of 3-fluoro-4-nitro-1,1′-biphenyl AE4 (2.19 g, 9.07 mmol) and tert-butyl (5-aminopyridin-2-yl)carbamate (2.11 g, 10.1 mmol) in DMSO (10.0 mL), DIEA (5.00 mL, 30.2 mmol) was added and the mixture was stirred at 85° C. for 2 hrs. The reaction mixture was concentrated, aq. LiCl (100 mL, 3%) was added, and the resulting mixture was extracted with EtOAc (50 mL×3). The combined organic phase was washed with brine (50.0 mL), dried over anhydrous Na₂SO₄, filtered and concentrated to give tert-butyl (5-((4-nitro-[1,1′-biphenyl]-3-yl)amino)pyridin-2-yl)carbamate AE5 (2.65 g, 64.7% yield) as a red solid, which was used in the next step without purification. ¹H NMR (400 MHz, CDCl₃) δ=8.31-8.24 (m, 1H), 8.18-8.13 (m, 1H), 7.63-7.58 (m, 3H), 7.52-7.48 (m, 6H), 7.46-7.45 (m, 1H), 7.42-7.40 (m, 1H), 1.55-1.50 (s, 9H).

Step 3. To a solution of tert-butyl (5-((4-nitro-[1,1′-biphenyl]-3-yl)amino)pyridin-2-yl)carbamate AE5 (2.65 g, 6.52 mmol) in methanol (20.0 mL), Pd/C (2.74 g, wet, purity 10%) was added. The reaction mixture was degassed and purged with H₂ (×3) and stirred at 25° C. for 12 hrs under H₂ (15 psi) The mixture was filtered through celite and washed with MeOH/DCM (1/5, 50 mL×4). The filtrate was concentrated to give tert-butyl (5-((4-amino-[1,1′-biphenyl]-3-yl)amino)pyridin-2-yl)carbamate AE6 (1.84 g, 74.9% yield) as a yellow solid. LCMS Rₜ=0 547 min in 1 min chromatography, purity 32.2%, MS ESI calcd. for 376.19 [M+H]⁺ 377.19, found 377.15.

Step 4. To a solution of tert-butyl (5-((4-amino-[1,1′-biphenyl]-3-yl)amino)pyridin-2-yl)carbamate AE6 (1.84 g, 4.89 mmol) in acetic acid (10.0 mL), 2-aminonicotinaldehyde (649 mg, 5.31 mmol) was added under N₂, and the mixture was stirred at 100° C. for 12 hrs. The reaction mixture was concentrated and the resulting residue was purified by flash silica gel chromatography (eluent with 0-43% of EtOAc in PE) to give tert-butyl (5-(2-(2-aminopyridin-3-yl)-6-phenyl-1H-benzo[d]imidazol-1-yl)pyridin-2-yl)carbamate AE7 (1.20 g, 46.7% yield) as a yellow solid. LCMS Rₜ=0.462 min in 1 min chromatography, purity 77.4%, MS ESI calcd. for 478.21 [M+H-Boc]⁺ 379.21, found 379.2.

Step 5. A mixture of tert-butyl (5-(2-(2-aminopyridin-3-yl)-6-phenyl-1H-benzo[d]imidazol-1-yl)pyridin-2-yl)carbamate AE7 (1.20 g, 2.51 mmol) in methanol (2.00 mL) and HCl/dioxane (10.0 mL, 2.51 mmol) was stirred at 25° C. for 12 hrs. The reaction mixture was concentrated directly and the mixture was purified by prep-HPLC (Welch Ultimate XB-CN 250*50 * 10 μm: mobile phase: [Heptane -EtOH (0.1% NH₃H₂O)]; B %: 15%-45%, 15 min) to give 5-(2-(2-aminopyridin-3-yl)-6-phenyl-1H-benzo[d]imidazol-1-yl)pyridin-2-amine AE8 (0.140 g, 14.8% yield) as a yellow solid. ¹H NMR (400 MHz, CDC3) δ=8.17 (d, J=2.0 Hz, 1H), 8.07-8.05 (m, 1H), 7.89-7.85 (m, 1H), 7.63-7.56 (m, 4H), 7.46-7.40 (m, 3H), 7.37-7.34 (m, 2H), 7.34-7.33 (m, 1H), 7.22 (dd, J=1.6, 7.6 Hz, 1H), 6.65-6.57 (m, 3H), 6.44 (dd, J=4.8, 7.6 Hz, 1H). LCMS Rₜ=0.432 min in 1 min chromatography, purity 74.2%, MS ESI calcd. for 378.16 [M+H]⁺ 379.16, found 379.1.

Step 6. To a solution of 5-(2-(2-aminopyridin-3-yl)-6-phenyl-1H-benzo[d]imidazol-1-yl)pyridin-2-amine AE8 (1.00 g, 2.64 mmol) in pyridine (6.00 mL), methyl (1r,4r)-4-(chlorocarbonyl)cyclohexane-1-carboxylate AE2 (1.01 g, 5.27 mmol) was added and the mixture was stirred at 100° C. for 12 hrs. The residue was purified by flash silica gel chromatography (eluent with 0%-6% of MeOH in DCM) to give methyl (1r,4r)-4-((5-(2-(2-aminopyridin-3-yl)-6-phenyl-1H-benzo[d]imidazol-1-yl)pyridin-2-yl)carbamoyl)cyclohexane-1-carboxylate AE9 (150 mg, 3.63% yield) as a yellow solid. LCMS Rₜ=0.695 min in 1 min chromatography, purity 20.6%, MS ESI calcd. for 546.24 [M+H]⁺ 547.24, found 547.2.

Step 7. To a solution of methyl (1r,4r)-4-((5-(2-(2-aminopyridin-3-yl)-6-phenyl-1H-benzo[d]imidazol-1-yl)pyridin-2-yl)carbamoyl)cyclohexane-1-carboxylate AE9 (150 mg, 0.0960 mmol) in THF (2.00 mL), water (0.500 mL) and LiOH·H₂O (24.0 mg, 0.608 mmol) were added and the mixture was stirred at 25° C. for 12 hrs. The reaction mixture was concentrated directly and the resulting residue was purified by prep-HPLC (Column: Welch Xtimate C18 40*200 mm*7 μm; mobile phase: [water (FA)-ACN]; B %: 6%-46%, 30 min) to give (1r,4r)-4-((5-(2-(2-aminopyridin-3-yl)-6-phenyl-1H-benzo[d]imidazol-1-yl)pyridin-2-yl)carbamoyl)cyclohexane-1-carboxylic acid I-32 (4.50 mg, 3.91% yield) as a yellow solid. ¹H NMR (400 MHz, DMSO-d₆) δ=10.69 (s, 1H), 8.37 (d, J=2.4 Hz, 1H), 8.29 (d, J=8.8 Hz, 1H), 8.04-7.98 (m, 2H), 7.89 (d, J=8.4 Hz, 1H), 7.71-7.64 (m, 3H), 7.47-7.41 (m, 3H), 7.38-7.32 (m, 1H), 7.27 (dd, J=1.6, 7.6 Hz, 1H), 6.92 (s, 2H), 6.47 (dd, J=4.8, 7.6 Hz, 1H), 2.56-2.53 (m, 1H), 2.24-2.16 (m, 1H), 2.00-1.95 (m, 2H), 1.93-1.87 (m, 2H), 1.53-1.42 (m, 2H), 1.39-1.29 (m, 2H). HPLC Rₜ=2.726 min in 8 min chromatography, purity 93.8%. LCMS Rₜ=1.236 min in 2 min chromatography, purity 90.8%, MS ESI calcd. for 532.22 [M+H]⁺ 533.22, found 533.0.

### Example 33. Synthesis of (1r,4r)-4-((4-(2-(2-aminopyridin-3-yl)-6-morpholino-1H-benzo[d]imidazol-1-yl)phenyl)carbamoyl)cyclohexane-1-carboxylic acid (Compound I-33)

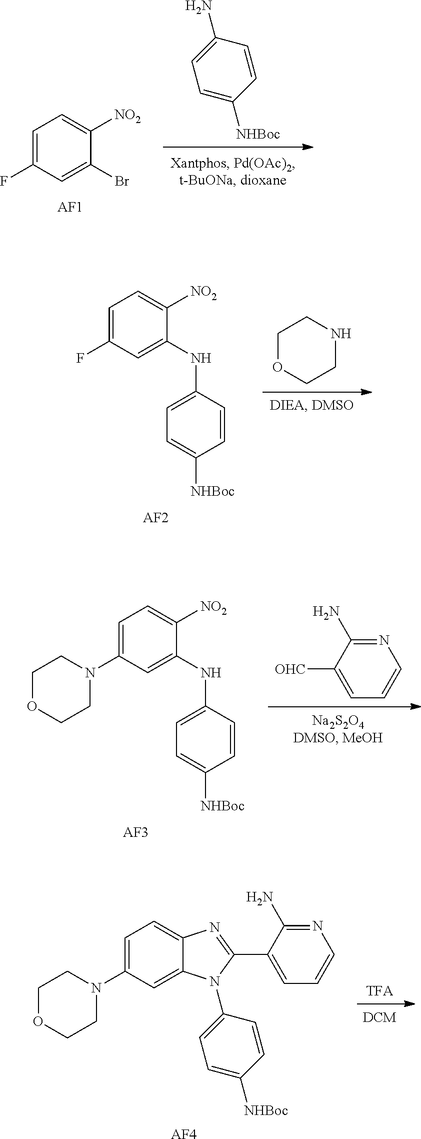

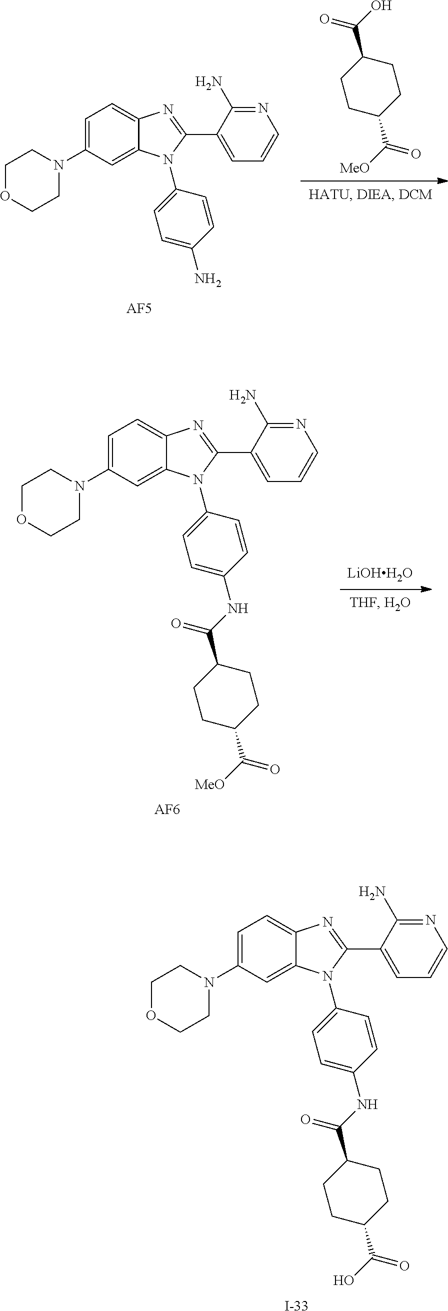

Step 1. A solution of Pd(OAc)₂ (0.510 g, 2.27 mmol) and Xantphos (1.32 g, 2.27 mmol) in 1,4-dioxane (70.0 mL) was stirred at 25° C. for 15 mins. The mixture was added to a solution of 2-bromo-4-fluoro-1-nitro-benzene AF (5.00 g, 22.7 mmol), tert-butyl N-(4-aminophenyl)carbamate (5.68 g, 27.3 mmol) and t-BuONa (3.28 g, 34.1 mmol) in 1,4-dioxane (70.0 mL), and the resulting mixture stirred at 100° C. for 12 hrs. Water (50 mL) was added, and the reaction mixture was extracted with EtOAc (200 mL×3). The combined organic phase was washed with brine (50 mL), dried over anhydrous Na₂SO₄, filtered and concentrated. The residue was purified by flash silica gel chromatography (eluent with 0-17% of EtOAc in PE) to give tert-butyl N-[4-(5-fluoro-2-nitro-anilino)phenyl]carbamate AF2 (270 mg, 34.2% yield) as a brown oil. ¹H NMR (400 MHz, DMSO-d₆) δ=9.58-9.43 (m, 2H), 8.22 (dd, J=6.4, 9.6 Hz, 1H), 7.54 (d, J=8.8 Hz, 2H), 7.24 (d, J=8.8 Hz, 2H), 6.71-6.56 (m, 2H), 1.51-1.46 (s, 9H).

Step 2. To a solution of tert-butyl N-[4-(5-fluoro-2-nitro-anilino)phenyl]carbamate AF2 (6.24 g, 18.0 mmol) in DMSO (80.0 mL), DIEA (2.32 g, 18.0 mmol) and morpholine (1.57 g, 18.0 mmol) were added and the mixture was stirred at 100° C. for 12 hrs. Water (50 mL) was added, and the reaction mixture was extracted with EtOAc (200 mL×2). The combined organic phase was washed with brine (50 mL), dried over anhydrous Na₂SO₄, filtered and concentrated to give tert-butyl N-[4-(5-morpholino-2-nitro-anilino)phenyl]carbamate AF3 (6.19 g, 83.1% yield) as a yellow solid, which was used in the next step without further purification. ¹H NMR (400 MHz, DMSO-d₆) δ=9.68 (s, 1H), 9.43 (s, 1H), 7.99 (d, J=9.6 Hz, 1H), 7.51 (d, 0.1=8.8 Hz, 2H), 7.26 (d, J=8.8 Hz, 2H), 6.54 (dd, J=2.4, 10.0 Hz, 1H), 6.23 (d, J=2.4 Hz, 1H), 3.67-3.63 (m, 4H), 3.23-3.19 (m, 4H), 1.48 (s, 9H).

Step 3. To a solution of tert-butyl N-[4-(5-morpholino-2-nitro-anilino)phenyl]carbamate AF3 (1.50 g, 3.62 mmol) and 2-aminopyridine-3-carbaldehyde (0.600 g, 4.89 mmol) in DMSO (10.0 mL) and methanol (10.0 mL), Na₂S₂O₄ (1.89 g, 10.9 mmol) was added under N₂ and the mixture was stirred at 100° C. for 12 hrs. Water (10 mL) was added, and the reaction mixture was extracted with EtOAc (40 mL×3). The combined organic phase was washed with brine (10 mL), dried over anhydrous Na₂SO₄, filtered and concentrated to give tert-butyl N-[4-[2-(2-amino-3-pyridyl)-6-morpholino-benzimidazol-1-yl]phenyl]carbamate AF4 (2.48 g, crude) as a yellow solid, which was used in the next step without further purification. ¹H NMR (400 MHz, DMSO-d) δ 9.67 (s, 1H), 7.92 (dd, J=1.6, 4.8 Hz, 1H), 7.63 (d, J=8.4 Hz, 3H), 7.28 (d, J=8.8 Hz, 2H), 7.13 (br s, 1H), 7.09-7.05 (m, 2H), 6.77-6.70 (m, 1H), 6.51 (d, 0.1=2.0 Hz, 1H), 6.35 (dd, J=4.8, 7.6 Hz, 1H), 3.73-3.70 (m, 4H), 3.05-3.02 (m, 4H), 1.50 (s, 9H).

Step 4. To a solution of tert-butyl N-[4-[2-(2-amino-3-pyridyl)-6-morpholino-benzimidazol-1-yl]phenyl]carbamate AF4 (480 mg, 0.986 mmol) in DCM (5.00 mL), TFA (1.00 mL, 13.1 mmol) was added and the mixture was stirred at 25° C. for 2 hrs. The reaction mixture was concentrated directly to give 3-[1-(4-aminophenyl)-6-morpholino-benzimidazol-2-yl]pyridin-2-amine AF5 (380 mg, 99.7% yield) as a brown oil, which was used in the next step without further purification. LCMS Rₜ=0.394 min in 1.0 min chromatography, purity 76.9%, MS ESI calcd. for 386.19 [M+H]⁺ 387.19, found 387.2.

Step 5. To a solution of 3-[1-(4-aminophenyl)-6-morpholino-benzimidazol-2-yl]pyridin-2-amine AF5 (380 mg, 0.983 mmol) in DCM (5.00 mL), DIEA (381 mg, 2.95 mmol) and trans-4-methoxycarbonylcyclohexanecarboxylic acid (183 mg, 0.983 mmol) were added. HATU (449 mg, 1.18 mmol) was then added, and the mixture was stirred at 25° C. for 2 hrs. The reaction mixture was filtered, and the filtrate was concentrated to dryness. The residue was purified by flash silica gel chromatography (eluent with 0-78% of EtOAc in PE) to give trans-methyl 4-[[4-[2-(2-amino-3-pyridyl)-6-morpholino-benzimidazol-1-yl]phenyl]carbamoyl]cyclohexanecarboxylate AF6 (180 mg, 33.0% yield) as a brown oil. LCMS Rₜ=0.506 min in 1.0 min chromatography, purity 64.3%, MS ESI calcd. for 554.26 [M+H]⁺ 555.26, found 555.3.

Step 6. To a solution of trans-methyl 4-[[4-[2-(2-amino-3-pyridyl)-6-morpholino-benzimidazol-1-yl]phenyl]carbamoyl]cyclohexanecarboxylate AF6 (150 mg, 0.270 mmol) in THF (2.00 mL), LiOH·H₂O (34.0 mg, 0.811 mmol) and water (0.500 mL) were added and the mixture was stirred at 25° C. for 12 hrs. The reaction mixture was filtered, and the filtrate was concentrated to dryness. The residue was purified by prep-HPLC (Column: Welch Xtimate C18 40*200 mm*7 μm, mobile phase: [water (NH₄HCO₃)-ACN]; B %: 0%-36%, 25 min) to give trans-4-[[4-[2-(2-amino-3-pyridyl)-6-morpholino-benzimidazol-1-yl]phenyl]carbamoyl]cyclohexanecarboxylic acid I-33 (46.6 mg, 29.8% yield) as a yellow solid. ¹H NMR (400 MHz, DMSO-d₆) δ=10.12 (s, 1H), 7.92 (dd, J=20, 4.8 Hz, 1H), 7.78 (d, J=8.8 Hz, 2H), 7.64 (d, J=9.2 Hz, 1H), 7.31 (d, J=8.8 Hz, 2H), 7.13-7.02 (m, 4H), 6.52 (d, J=2.0 Hz, 1H), 6.36 (dd, J=4.8, 7.6 Hz, 1H), 3.75-3.68 (m, 4H), 3.07-3.00 (m, 4H), 2.37-2.30 (m, 1H), 2.26-2.16 (m, 1H), 2.02-1.87 (m, 4H), 1.54-1.43 (m, 2H), 1.41-1.30 (m, 2H). HPLC Rₜ=2.011 min in 8 min chromatography, purity 93.6%. LCMS Rₜ=0.941 min in 2.0 min chromatography, purity 93.9%, MS ESI calcd. for 540.25 [M+H]⁺ 541.25, found 541.0.

### Example 34. Synthesis of (1r,4r)-4-((4-(2-(2-aminopyridin-3-yl)-6-(tetrahydro-2H-pyran-4-yl)-1H-benzo[d]imidazol-1-yl)phenyl)carbamoyl)cyclohexane-1-carboxylic acid (Compound I-34)

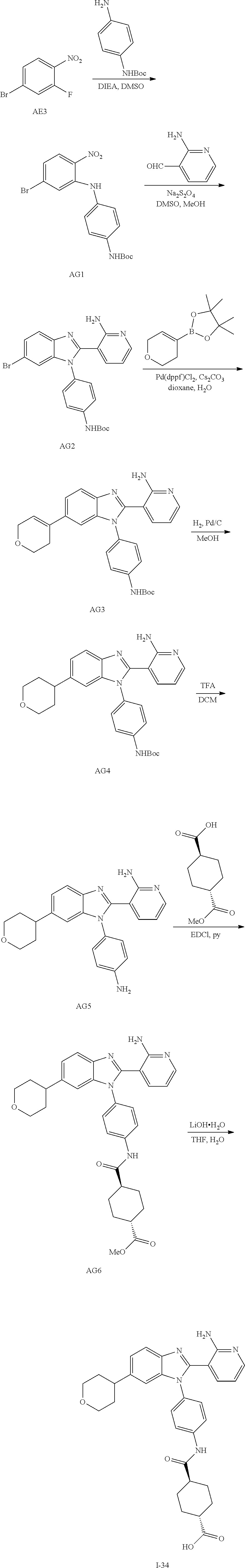

Step 1. To a solution of 4-bromo-2-fluoro-1-nitro-benzene AE3 (5.00 g, 22.7 mmol) in DMSO (50 mL), DIEA (2.94 g, 22.7 mmol) and tert-butyl N-(4-aminophenyl)carbamate (4.73 g, 22.7 mmol) were added and the mixture was stirred at 80° C. for 12 hrs. Water (50 mL) was added, and the reaction mixture was extracted with EtOAc (150 mL×2). The combined organic phase was washed with brine (50 mL), dried over anhydrous Na₂SO₄, filtered and concentrated to give tert-butyl N-[4-(5-bromo-2-nitro-anilino)phenyl]carbamate AG1 (9.40 g, crude) as a brown solid, which was used in the next step without further purification. ¹H NMR (400 MHz, DMSO-d₆) δ=9.52-9.40 (m, 2H), 8.03 (d, J=9.2 Hz, 1H), 7.54 (br d, J=8.8 Hz, 2H), 7.23 (d, J=8.8 Hz, 2H), 7.03-6.92 (m, 2H), 1.49 (s, 9H).

Step 2. To a solution of tert-butyl N-[4-(5-bromo-2-nitro-anilino)phenyl]carbamate AG1 (8.90 g, 21.8 mmol) and 2-aminopyridine-3-carbaldehyde (3.59 g, 29.4 mmol) in DMSO (100 mL) and methanol (100 mL), Na₂S₂O₄ (7.59 g, 43.6 mmol) was added under N₂ and the mixture was stirred at 100° C. for 12 hrs water (100 mL) was added, and the reaction mixture was extracted with EtOAc (300 mL×2). The combined organic phase was washed with brine (50 mL), dried over anhydrous Na₂SO₄, filtered and concentrated to give tert-butyl N-[4-[2-(2-amino-3-pyridyl)-6-bromo-benzimidazol-1-yl]phenyl]carbamate AG2 (9.60 g, 91.7% yield) as a brown oil, which was used in the next step without further purification. ¹H NMR (400 MHz, DMSO-d₆) δ=9.67 (s, 1H), 7.99-7.96 (m, 1H), 7.75 (d, J=8.4 Hz, 1H), 7.63 (d, J=8.8 Hz, 2H), 7.46 (dd, J=2.0, 8.41 Hz, 1H), 7.33 (d, J=8.8 Hz, 2H), 7.25 (d, J=2.0 Hz, 1H), 7.17 (dd, J=1.8, 7.6 Hz, 1H), 7.04 (s, 2H), 6.40 (dd, J=4.8, 7.6 Hz, 1H), 1.50 (s, 9H).

Step 3. A mixture of tert-butyl N-[4-[2-(2-amino-3-pyridyl)-6-bromo-benzimidazol-1-yl]phenyl]carbamate AG2 (800 mg, 1.67 mmol), 2-(3,6-dihydro-2H-pyran-4-yl)-4,4,5,5-tetramethyl-1,3,2-dioxaborolane (420 mg, 2.00 mmol), Cs₂CO₃ (1.63 g, 5.00 mmol), water (3.0 mL) in 1,4-dioxane (10.0 mL) was degassed and purged with N₂ (×3). Pd(dppf)Cl₂ (122 mg, 0.167 mmol) was then added to the mixture, and the mixture was stirred at 80° C. for 12 hrs under N₂ atmosphere. Water (5 mL) was added, and the reaction mixture was extracted with EtOAc (20 mL×3). The combined organic phase was washed with brine (5 mL), dried over anhydrous Na₂SO₄, filtered and concentrated. The residue was purified by flash silica gel chromatography (eluent with 0-3% of MeOH in DCM) to give tert-butyl N-[4-[2-(2-amino-3-pyridyl)-6-(3,6-dihydro-2H-pyran-4-yl)benzimidazol-1-yl]phenyl]carbamate AG3 (570 mg, 70.8% yield) as a yellow oil. ¹H NMR (400 MHz, DMSO-d₆) δ=9.67 (s, 1H), 7.96 (dd, J=1.6, 4.8 Hz, 1H), 7.74 (d, J=8.4 Hz, 1H), 7.64 (d, J=8.8 Hz, 2H), 7.46 (dd, J=1.6, 8.4 Hz, 1H), 7.32 (d, J=8.8 Hz, 2H), 7.17-7.06 (m, 4H), 6.38 (dd, J=4.8, 7.6 Hz, 1H), 6.20 (br s, 1H), 4.20 (br d, J=2.8 Hz, 2H), 3.79 (t, J=5.6 Hz, 2H), 2.43 (br d, 0.1=1 6 Hz, 2H), 1.50 (s, 9H).

Step 4. To a solution of tert-butyl N-[4-[2-(2-amino-3-pyridyl)-6-(3,6-dihydro-2H-pyran-4-yl)benzimidazol-1-yl]phenyl]carbamate AG3 (490 mg, 1.01 mmol) in methanol (10.0 mL), wet Pd/C (10%, 150 mg) and Pd(OH)₂ (20%, 150 mg) was added under a N₂ atmosphere. The suspension was degassed and purged with H₂ (×3), and the reaction mixture was stirred under H₂ (40 psi) at 25° C. for 12 hrs. The reaction mixture was filtered and the filtrate was concentrated to give tert-butyl N-[4-[2-(2-amino-3-pyridyl)-6-tetrahydropyran-4-yl-benzimidazol-1-yl]phenyl]carbamate AG4 (490 mg, 99.6% yield) as a yellow oil, which was used in the next step without further purification. ¹H NMR (400 MHz, DMSO-d₆) δ=9.67 (s, 1H), 7.95 (s, 1H), 7.73-7.68 (m, 2H), 7.39-7.36 (m, 1H), 7.31-7.28 (m, 2H), 7.23 (dd, J=1.6, 8.4 Hz, 1H), 7.14-7.07 (m, 3H), 6.97 (s, 1H), 6.38 (dd, J=4.8, 7.6 Hz, 1H), 4.11 (br d, J=3.6 Hz, 2H), 3.91 (br d, J=2.4 Hz, 2H), 1.70-1.64 (m, 5H), 1.50 (s, 9H).

Step 5. To a solution of tert-butyl N-[4-[2-(2-amino-3-pyridyl)-6-tetrahydropyran-4-yl-benzimidazol-1-yl]phenyl]carbamate AG4 (490 mg, 1.01 mmol) in DCM (5.00 ml), TFA (1.10 mL, 14.1 mmol) was added and the mixture was stirred at 25° C. for 2 hrs. The reaction mixture was filtered and the filtrate was concentrated to give 3-[1-(4-aminophenyl)-6-tetrahydropyran-4-yl-benzimidazol-2-yl]pyridin-2-amine AGS (388 mg, 99.8% yield) as a yellow oil, which was used in the next step without further purification. LCMS Rₜ=0.424 min in 1.0 min chromatography, purity 63.3%, MS ESI calcd. for 385.19 [M+H]⁺ 385.19, found 386.2.

Step 6. To a solution of 3-[1-(4-aminophenyl)-6-tetrahydropyran-4-yl-benzimidazol-2-yl]pyridin-2-amine AG5 (388 mg, 1.01 mmol) and trans-4-methoxycarbonylcyclohexanecarboxylic acid (187 mg, 1.01 mmol) in pyridine (5.00 mL), EDCI (289 mg, 1.51 mmol) was added and the mixture was stirred at 50° C. for 12 hrs. Water (5 mL) was added, and the resulting mixture was extracted with EtOAc (20 mL×2). The combined organic phase was washed with brine (5 mL×2), dried over anhydrous Na₂SO₄, filtered and concentrated. The residue was purified by flash silica gel chromatography (eluent with 0-3% of MeOH in DCM) to give trans-methyl 4-[[4-[2-(2-amino-3-pyridyl)-6-tetrahydropyran-4-yl-benzimidazo)-1-yl]phenyl]carbamoyl]cyclohexanecarboxylate AG6 (170 mg, 16.8% yield) as a yellow oil. LCMS Rₜ=0.526 min in 1.0 min chromatography, purity 68.9%, MS ESI calcd. for 553.27 [M+H]⁺ 554.27, found 554.3.

Step 7. To a solution of trans-methyl 4-[[4-[2-(2-amino-3-pyridyl)-6-tetrahydropyran-4-yl-benzimidazol-1-yl]phenyl]carbamoyl]cyclohexanecarboxylate AG6 (170 mg, 0.307 mmol) in THF (2.00 mL), LiOH·H₂O (39.0 mg, 0.921 mmol) and water (0.500 mL) were added and the mixture was stirred at 25° C. for 12 hrs. The reaction mixture was filtered, and the filtrate was concentrated. The residue was purified by prep-HPLC (Column: Welch Xtimate C18 40*200 mm*7 μm; mobile phase: [water (NH₄HCO₃)-ACN]; B %: 0% -38%, 25 min) to give trans-4-[[4-[2-(2-amino-3-pyridyl)-6-tetrahydropyran-4-yl-benzimidazol-1-yl]phenyl]carbamoyl]cyclohexanecarboxylic acid I-34 (16.0 mg, 76.9% yield) as a white solid. ¹H NMR (400 MHz, DMSO-d₆) δ=10.12 (s, 1H), 7.95 (dd, J=1.6, 4.8 Hz, 1H), 7.79 (d, J=8.8 Hz, 2H), 7.71 (d, 0.1=8.4 Hz, 1H), 7.33 (d, J=8.8 Hz, 2H), 7.24 (dd, J=1.6, 8.4 Hz, 1H), 7.14 (dd, J=1.6, 7.6 Hz, 1H), 7.07-6.98 (m, 3H), 6.39 (dd, J=4.8, 7.6 Hz, 1H), 3.92 (br d, J=10.4 Hz, 2H), 3.45-3.39 (m, 2H), 2.91-2.80 (m, 1H), 2.33 (br d, J=3.2 Hz, 1H), 2.26-2.17 (m, 1H), 2.02-1.87 (m, 4H), 1.71-1.63 (m, 4H), 1.54-1.31 (m, 4H). HPLC Rₜ=2.419 min in 8 min chromatography, purity 97.4%. LCMS Rₜ=0.707 min in 2.0 min chromatography, purity 100%, MS ESI calcd. for 539.25 [M+H]⁺ 540.25, found 540.3.

### Example 35. Synthesis of (1r,4r)-4-((4-(2-(2-aminopyridin-3-yl)-6-(tert-butyl)-1H-benzo[d]imidazol-1-yl)phenyl)carbamoyl)cyclohexane-1-carboxylic acid (Compound I-35)

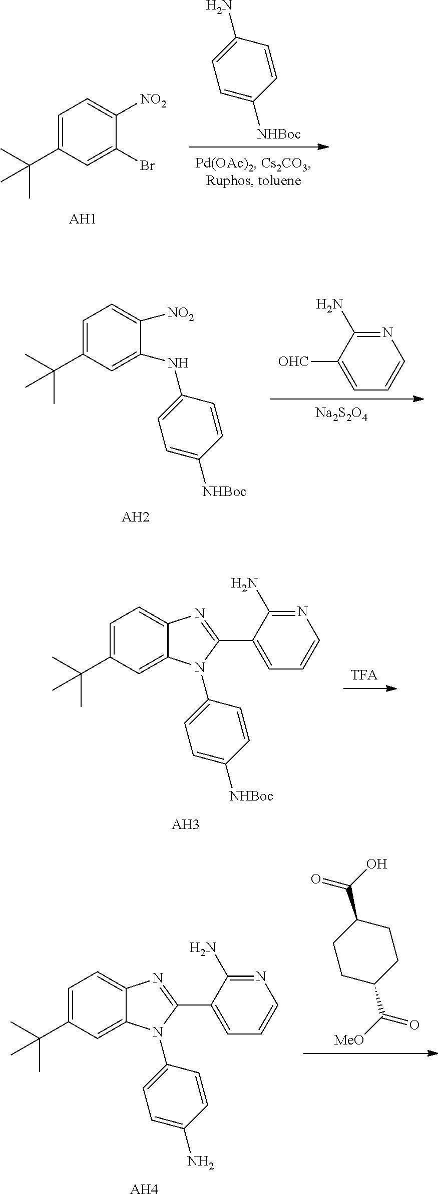

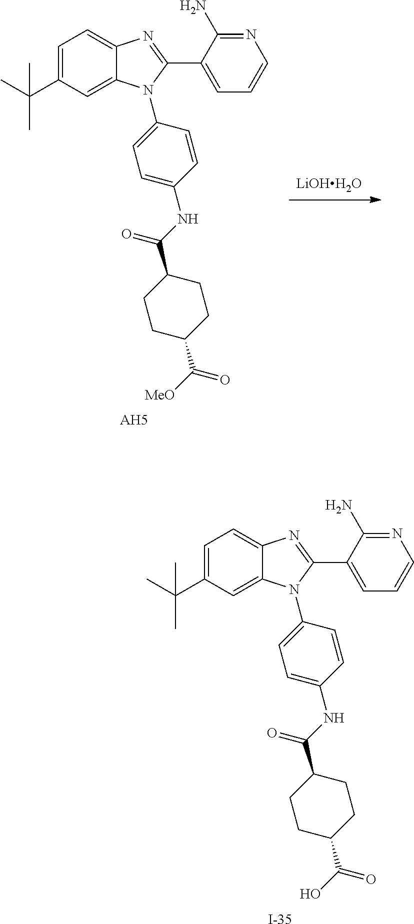

Step 1. To a solution of tert-butyl N-(4-aminophenyl)carbamate (1.94 g, 9.30 mmol) in toluene (30 mL), 2-bromo-4-tert-butyl-1-nitro-benzene AH1 (2.40 g, 9.30 mmol), Cs₂CO₃ (9.08 g, 27.9 mmol), Ruphos (665 mg, 1.39 mmol) and Pd(OAc)₂ (209 mg, 0.930 mmol) were added at 25° C., and the mixture was stirred at 110° C. for 14 hrs under N₂. The reaction was diluted with EtOAc (100 mL) and washed with 50 mL water and brine (3×50 mL). The organics were then separated and dried over Na₂SO₄, filtered, and concentrated to dryness. The crude was then purified by flash column chromatography eluting with 40% of EtOAc in PE to afford tert-butyl N-[4-(5-tert-butyl-2-nitro-anilino)phenyl]carbamate A112 (800 mg, 22.3% yield) as a brown solid. ¹H NMR (400 MHz, DMSO-d₆) δ=9.46 (s, 2H), 8.10 (d, J=8.8 Hz, 1H), 7.58 (d, J=8.8 Hz, 2H), 7.30 (d, J=8.8 Hz, 2H), 7.12 (d, J=1.6 Hz, 1H), 6.96 (dd, J=9.2, 2.0 Hz, 1H), 1.54 (s, 9H), 1.22 (s, 9H).

Step 2. To a solution of tert-butyl N-[4-(5-tert-butyl-2-nitro-anilino)phenyl]carbamate AH2 (600 mg, 1.56 mmol) and 2-aminopyridine-3-carbaldehyde (190 mg, 1.56 mmol) in DMSO (10.0 mL) and methanol (10.0 mL), Na₂S₂O₄ (1.36 g, 7.78 mmol) was added and the mixture was stirred at 100° C. for 12 hrs. The reaction was diluted with EtOAc (100 mL) and washed with 50 mL water and brine (3×50 mL). The organics were then separated and dried over Na₂SO₄, filtered, and concentrated. The crude was purified by flash column chromatography eluting with 40% of EtOAc in PE to afford tert-butyl N-[4-[2-(2-amino-3-pyridyl)-6-tert-butyl-benzimidazol-1-yl]phenyl]carbamate AH3 (500 mg, 70.2% yield) as a yellow solid. ¹H NMR (400 MHz, DMSO-d₆) δ=9.74 (s, 1H), 8.01 (dd, J=4.8, 1.6 Hz, 1H), 7.77 (d, J=8.4 Hz, 1H), 7.70 (d, J=8.8 Hz, 2H), 7.47 (dd, J=8.4, 1.6 Hz, 1H), 7.37 (d, J=8.8 Hz, 2H), 7.17 (dd, J=7.6, 1.6 Hz, 1H), 7.09-7.15 (m, 3H), 6.44 (dd, J=7.6, 4.8 Hz, 1H), 1.56 (s, 9H), 1.35 (s, 9H).

Step 3. To a solution of tert-butyl N-[4-[2-(2-amino-3-pyridyl)-6-tert-butyl-benzimidazol-1-yl]phenyl]carbamate AH3 (250 mg, 0.546 mmol) in DCM (5.00 mL), TFA (0.500 mL) was added under N₂ atmosphere. The reaction mixture was degassed and purged with N₂ (×3), and the mixture was stirred at 25° C. for 6 hrs. The reaction mixture was concentrated to afford 3-[1-(4-aminophenyl)-6-tert-butyl-benzimidazol-2-yl]pyridin-2-amine AH4 (200 mg, crude) as a brown oil, which was used directly in the next step.

Step 4. To a solution of 3-[1-(4-aminophenyl)-6-tert-butyl-benzimidazol-2-yl]pyridin-2-amine AH4 (100 mg, 0.280 mmol) and trans-4-methoxycarbonylcyclohexanecarboxylic acid (52.0 mg, 0.280 mmol) in pyridine (2.00 mL), EDCI (258 mg, 0.560 mmol) was added and the mixture was stirred at 25° C. for 12 hrs. The reaction was diluted with EtOAc (100 mL) and washed with 50 mL water and brine (3×50 mL). The organics were then separated and dried over Na₂SO₄ filtered, and concentrated. The crude was then purified by flash column chromatography eluting with 80% of EtOAc in PE to afford trans-methyl 4-[[4-[2-(2-amino-3-pyridyl)-6-tert-butyl-benzimidazol-1-yl]phenyl]carbamoyl]cyclohexanecarboxylate A H5 (100 mg, 37.4% yield) as a brown oil. LCMS Rₜ=0.558 min in 4 min chromatography, Xtimate C18 2.1*30 mm, 3 μm, purity 77%, MS ESI calcd. for 525.27 [M+H]⁺ 526.27, found 5262.

Step 5. To a solution of trans-methyl 4-[[4-[2-(2-amino-3-pyridyl)-6-tert-butyl-benzimidazol-1-yl]phenyl]carbamoyl]cyclohexanecarboxylate AH5 (100 mg, 0.190 mmol) in water (0.500 mL) and THF (2.00 mL), LiOH·H₂O (166 ng, 0.951 mmol) was added and the mixture was stirred at 25° C. for 12 hrs. The crude product was purified by prep-HPLC (column: Welch Xtimate C18 200*40 mm*7 μm; mobile phase: [water (HCl)-ACN], B %: 10%-50%, 20 min) to afford trans-4-[[4-[2-(2-amino-3-pyridyl)-6-tert-butyl-benzimidazol-1-yl]phenyl]carbamoyl]cyclohexane carboxylic acid 1-35 (12.0 mg, 12.14% yield) as a yellow solid. ¹H NMR (400 MHz, DMSO-d₆) δ=10.20 (s, 1H), 8.05 (dd, J=6.0, 1.6 Hz, 1H), 7.86-7.75 (m, 3H), 7.68-7.62 (m, 1H), 7.55-7.49 (m, 1H), 7.44 (d, J=8.8 Hz, 2H), 7.15 (s, 1H), 6.79 (s, 1H), 2.43-2.35 (m, 1H), 2.28-2.15 (m, 1H), 2.05-1.85 (m, 4H), 1.56-1.34 (m, 4H), 1.31 (s, 9H). HPLC Rₜ=2.910 min in 4 min chromatography, ACE Excel 3 C18 4.6*100 mm, purity 98.6%. LCMS Rₜ=1.964 min in 4 min chromatography, Xtimate C18 2 1*30 mm, 3 m, purity 99.1%, MS ESI calcd. for 511.26 [M+H]⁺ 512.26, found 512.2.

### Example 36. Synthesis of (1s,4s)-4-((3-(4-(2-(2-aminopyridin-3-yl)-5-phenyl-3H-imidazo[4,5-b]pyridin-3-yl)phenyl)azetidin-1-yl)methyl)cyclohexane-1-carboxylic acid (Compound I-36)

### Synthesis of methyl (1s,4s)-4-formylcyclohexane-1-carboxylate (AI2)

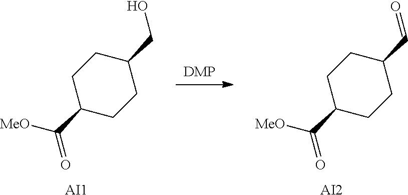

Step 1. To a solution of methyl (1s,4s)-4-(hydroxymethyl)cyclohexane-1-carboxylate A11 (200 mg, 1.16 mmol) in DCM (5.00 mL), DMP (985 mg, 2.32 mmol) was added at 0° C. After stirring at 25° C. for 2 hrs, the mixture was quenched with sat. aq. Na₂S₂O₃ (20 mL) and sat. aq. NaHCO₃ (20 mL), then stirred for 30 mins. The mixture was extracted with DCM (20 mL×3) and the organic phase was washed with brine (20.0 mL), dried with Na₂SO₄, filtered, and concentrated to give methyl (1s,4s)-4-formylcyclohexane-1-carboxylate A12 (200 mg, crude) as a white solid, which was used in the next step directly.

### Synthesis of (1s,4s)-4-((3-(4-(2-(2-aminopyridin-3-yl)-5-phenyl-3H-imidazo[4,5-b]pyridin-3-yl)phenyl)azedin-1-yl)methyl)cyclohexane-1-carbocylic acid (Compound I-36)

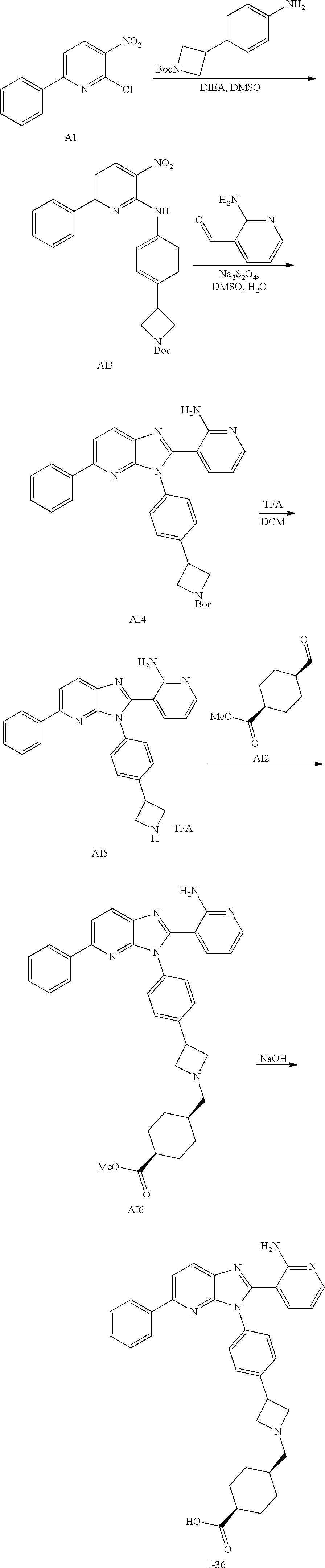

Step 1. To a solution of 2-chloro-3-nitro-6-phenylpyridine A1 (1.80 g, 7.67 mmol) in DMSO (15.0 mL), tert-butyl 3-(4-aminophenyl)azetidine-1-carboxylate (2.00 g, 8.05 mmol) and DIEA (2.10 mL, 12.3 mmol) were added at 25° C., and the mixture was stirred at 80° C. for 16 hrs. Water (200 mL) was added, and the resulting mixture was extracted with EtOAc (20 mL×3). The combined organic phase was washed with brine (50 mL), water (50 mL), dried over anhydrous Na₂SO₄, filtered and concentrated. The residue was purified by flash silica gel chromatography (eluent with 0-33% of EtOAc in PE) to give tert-butyl 3-(4-((3-nitro-6-phenylpyridin-2-yl)amino)phenyl)azetidine-1-carboxylate A13 (1.20 g, 67.3% yield) as a red solid. ¹H NMR (400 MHz, CDCl₃) δ=10.31 (s, 1H), 8.59 (d, J=8.8 Hz, 1H), 8.10-8.00 (m, 2H), 7.76 (d, J=8.4 Hz, 2H), 7.57-7.45 (m, 3H), 7.38 (d, J=8.4 Hz, 2H), 7.31 (d, J=8.8 Hz, 1H), 4.36 (t, J=8.8 Hz, 2H), 4.02 (dd, J=8.4, 6.0 Hz, 2H), 3.82-3.73 (m, 1H), 1.49 (s, 9H).

Step 2. To a solution of tert-butyl 3-(4-((3-nitro-6-phenylpyridin-2-yl)amino)phenyl)azetidine-1-carboxylate A3 (2.30 g, 5.15 mmol) in DMSO (23.0 mL) and methanol (23.0 mL), 2-aminonicotinaldehyde (849 mg, 6.95 mmol) and Na₂S₂O₄ (1.79 g, 10.3 mmol) were added at 25° C., and the mixture was stirred at 100° C. for 16 hrs. The reaction mixture was concentrated to remove MeOH, water (50 mL) was added to the residue, and the resulting mixture was extracted with EtOAc (100 mL×3). The combined organic phase was washed with brine (20 mL), water (20 mL), dried over anhydrous Na₂SO₄, filtered and concentrated. The residue was purified by flash silica gel chromatography (eluent with 0-66% of EtOAc in PE) to give tert-butyl 3-(4-(2-(2-aminopyridin-3-yl)-5-phenyl-3H-imidazo[4,5-b]pyridin-3-yl)phenyl)azetidine-1-carboxylate A14 (400 mg, 17.1% yield) as a yellow oil. ¹H NMR (400 MHz, DMSO-d₆) δ=8.74 (d, J=2.4 Hz, 1H), 8.71 (d, J=2.4 Hz, 1H), 8.34-8.28 (m, 3H), 8.08 (dd, J=8.4, 2.0 Hz, 2H), 7.94 (dd, J=4.8, 2.0 Hz, 1H), 7.51 (d, J=8.4 Hz, 2H), 7.39 (dd, J=8.4, 6.4 Hz, 4H), 7.13 (dd, J=7.6, 1.6 Hz, 1H), 6.49-6.43 (m, 1H), 3.17 (s, 2H), 3.16 (s, 2H), 3.05 (s, 1H), 1.40 (s, 9H).

Step 3. To a solution of tert-butyl 3-(4-(2-(2-aminopyridin-3-yl)-5-phenyl-3H-imidazo[4,5-b]pyridin-3-yl)phenyl)azetidine-1-carboxylate AI4 (200 mg, 0.390 mmol) in DCM (4.00 mL), TFA (2.00 mL) was added at 25° C. and the mixture was stirred for 2 hrs. The reaction mixture was concentrated to give 3-(3-(4-(azetidin-3-yl)phenyl)-5-phenyl-3H-imidazo[4,5-b]pyridin-2-yl)pyridin-2-amine trifluoroacetate AI5 (161 mg, 99.8% yield) as a brown oil, which was used in the next step directly. LCMS Rₜ=0.410 min in 1 min chromatography, purity 79.5%, MS ESI calcd. for 418.19 [M+H]⁺ 419.19, found 419.1.

Step 4. To a solution of 3-(3-(4-(azetidin-3-yl)phenyl)-5-phenyl-3H-imidazo[4,5-b]pyridin-2-yl)pyridin-2-amine AI5 (160 mg, 0.382 mmol) in DMF (10.0 mL), TEA (116 mg, 1.15 mmol) was added. Methyl (1s,4s)-4-formylcyclohexane-1-carboxylate A12 (195 mg, 1.15 mmol) was then added and the mixture was stirred at 25° C. for 16 hrs. NaBH(OAc). (405 mg, 1.91 mmol) was subsequently added to the mixture and the mixture was stirred at 25° C. for 16 hrs. The mixture was quenched with water (20 mL) and extracted with EtOAc (30 mL×3). The organic phase was washed with brine (10 mL×2), dried with Na₂SO₄, filtered, and concentrated. The mixture was purified by column (MeOH in DCM, 0-20%) to give methyl (1s,4s)-4-((3-(4-(2-(2-aminopyridin-3-yl)-5-phenyl-3H-imidazo[4,5-b]pyridin-3-yl)phenyl)azetidin-1-yl)methyl)cyclohexane-1-carboxylate AI6 (80.0 mg, 36.5% yield) as a yellow solid LCMS Rₜ=0.46 min in 1.5 min chromatography, 5-95AB_220&254_Agil, purity 54.0%, MS ESI calcd. for 572.29 [M+H]⁺ 573.29, found 573.2.

Step 5. To a solution of methyl (1s,4s)-4-((3-(4-(2-(2-aminopyridin-3-yl)-5-phenyl-3H-imidazo[4,5-b]pyridin-3-yl)phenyl)azetidin-1-yl)methyl)cyclohexane-1-carboxylate AI6 (70.0 mg, 0.122 mmol) in water (1.00 mL) and THF (1.00 mL), NaOH (15.0 mg, 0.367 mmol) was added and the mixture was stirred at 25° C. for 16 hrs. The mixture was adjusted to pH ~6 and concentrated. The residue was purified by prep-HPLC (Xtimate C18 150*40 mm*5 μm, water (HCl)-ACN) to give (1s,4s)-4-((3-(4-(2-(2-aminopyridin-3-yl)-5-phenyl-3H-imidazo[4,5-b]pyridin-3-yl)phenyl)azetidin-1-yl)methyl)cyclohexane-1-carboxylic acid 1-36 (8.50 mg, 12.0% yield) as a yellow solid. ¹H NMR (DMSO-d₆ 400 MHz) δ_{H}=8.34 (d, J=8.4 Hz, 1H), 8.08 (dd, J=1.6, 6.0 Hz, 1H), 8.07-8.01 (m, 3H), 7.78 (d, J=7.2 Hz, 1H), 7.69-7.54 (m, 4H), 7.51-7.45 (m, 2H), 7.45-7.39 (m, 1H), 6.85-6.74 (m, 1H), 4.53-4.47 (m, 1H), 4.43-4.35 (m, 1H), 4.25-4.14 (m, 2H), 3.20 (d, J=6.4 Hz, 1H), 3.13 (d, J=6.8 Hz, 2H), 2.20-2.10 (m, 1H), 1.96-1.87 (m, 2H), 1.83-1.73 (m, 2H), 1.64-1.50 (m, 1H), 1.36-1.21 (m, 2H), 1.08-0.94 (m, 2H). HPLC Rₜ=8.340 min in 15 min chromatography, 0-60AB, purity 96.4%. LCMS Rₜ=2.290 min in 4 min chromatography, Xtimate C18 2.1*30 mm, 3 μm, purity 100%, MS ESI calcd. for 558.27 [M+H]⁺ 559.27, found 559.1.

### Example 37. Synthesis of 5-(3-(4-(2-(2-aminopyridin-3-yl)-5-phenyl-3H-imidazo[4,5-b]pyridin-3-yl)phenyl)azetidine-1-carbonyl)cyclohexane-1,3-dione (Compound I-37)

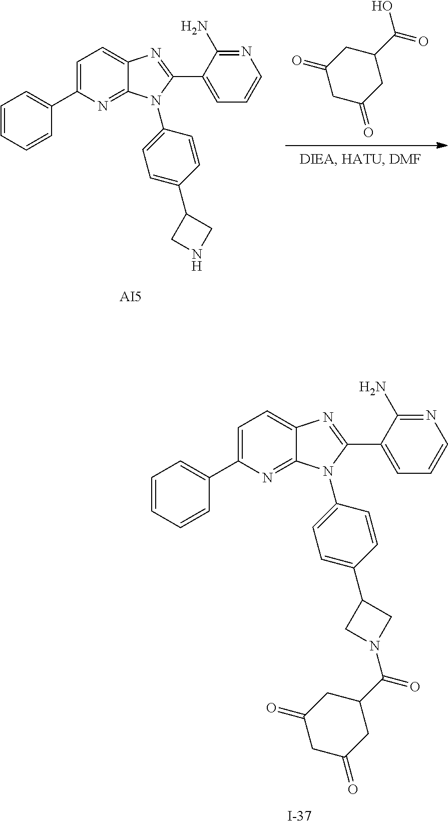

Step 1. To a solution of 3-(3-(4-(azetidin-3-yl)phenyl)-5-phenyl-3H-imidazo[4,5-b]pyridin-2-yl)pyridin-2-amine AI5 (170 mg, 0.410 mmol) and 3,5-dioxocyclohexane-1-carboxylic acid (95.0 mg, 0.610 mmol) in DMF (5.00 mL), DIEA (157 mg, 1.22 mmol) and HATU (232 mg, 0.610 mmol) were added and the mixture was stirred at 25° C. for 2 hrs. Water (10 ml) was added, and the resulting mixture was extracted with EtOAc (10 mL×3). The combined organic phase was washed with brine (10 mL), dried over anhydrous Na₂SO₄, filtered and concentrated. The residue was purified by prep-HPLC (column: Xtimate C18 150*40 mm*10 μm; mobile phase: [water (NH₄HCO₃)-ACN]; gradient: 2%-42% B over 32 min) to give 5-(3-(4-(2-(2-aminopyridin-3-yl)-5-phenyl-3H-imidazo[4,5-b]pyridin-3-yl)phenyl)azetidine-1-carbonyl)cyclohexane-1,3-dione I-37 (69.4 mg, 30.4% yield) as a white solid. ¹H NMR (400 MHz, DMSO-d₆) δ=8.30-8.24 (m, 1H), 8.08-7.95 (m, 4H), 7.61-7.54 (m, 2H), 7.53-7.43 (m, 4H), 7.42-7.36 (m, 1H), 7.24-7.19 (m, 1H), 6.94 (s, 2H), 6.43 (dd, J=4.8, 7.6 Hz, 1H), 5.18 (s, 1H), 4.71-4.57 (m, 1H), 4.41-4.25 (m, 2H), 4.24-3.86 (m, 3H), 3.17-3.02 (m, 1H), 3.00-2.83 (m, 1H), 2.47-2.18 (m, 3H).

### Example 38. Synthesis of N-(4-(2-(2-aminopyridin-3-yl)-5-phenyl-3H-imidazo[4,5-b]pyridin-3-yl)benzyl)-3,5-dioxocyclohexane-1-carboxamide (Compound I-38)

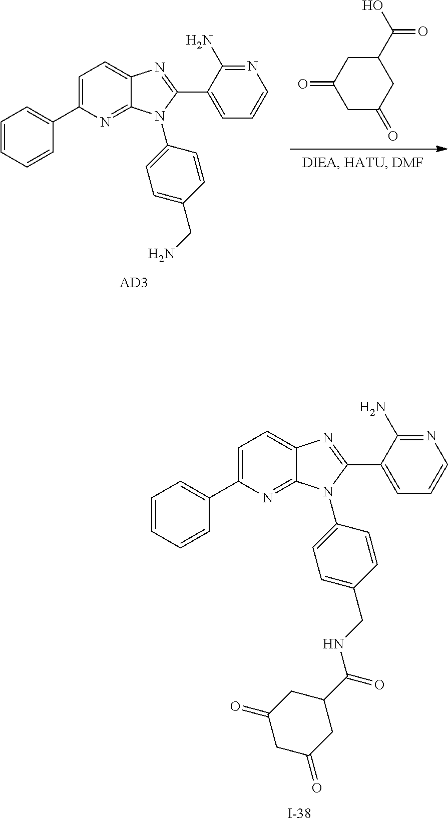

Step 1. To a solution of 3-(3-(4-(aminomethyl)phenyl)-5-phenyl-3H-imidazo[4,5-b]pyridin-2-yl)pyridin-2-amine AD3 (100 mg, 0.250 mmol) and 3,5-dioxocyclohexane-1-carboxylic acid (80.0 mg, 0.510 mmol) in DMF (3.00 mL), DIEA (99.0 mg, 0.760 mmol) and HATU (145 mg, 0.380 mmol) were added at 25° C., and the mixture was stirred for 2 hrs. The residue was purified by prep-HPLC (column: Xtimate C18 150*25 mm*5 μm; mobile phase: [water (FA)-ACN]; B %: 10%-50%) to give N-(4-(2-(2-aminopyridin-3-yl)-5-phenyl-3H-imidazo[4,5-b]pyridin-3-yl)benzyl)-3,5-dioxocyclohexane-1-carboxamide I-38 (21.2 mg, 15.6% yield) as a yellow solid. ¹H NMR (DMSO-d₆, 400 MHz,) δ=8.58 (t, J=5.6 Hz, 1H), 8.27 (d, J=8.4 Hz, 1H), 8.07-7.94 (m, 4H), 7.51-7.36 (m, 7H), 7.21 (dd, J=7.6, 1.6 Hz, 1H), 6.95 (s, 2H), 6.42 (dd, J=7.6, 4.8 Hz, 1H), 4.40 (d, J=6.0 Hz, 2H), 3.06-2.95 (m, 1H), 2.72-2.56 (m, 2H), 2.44-2.24 (m, 4H). HPLC Rₜ=2.088 min in 8 min chromatography, purity 99.6%. LCMS Rₜ=1.077 min in 2 min chromatography, purity 97.6%, MS ESI calcd. for 530.21 [M+H]+ 531.21, found 531.2.

### Example 39. Alternate Synthesis of 2-(1-(5-(2-(2-aminopyridin-3-yl)-5-phenyl-3H-imidazo[4,5-b]pyridin-3-yl)pyridin-2-yl)pyrrolidin-3-yl)-2-methylpropanoic acid (Compound I-39) Synthesis of methyl 2-methyl-2-pyrrolidin-3-yl-propanoate and methyl 2-pyrrolidin-3-ylpropanoate

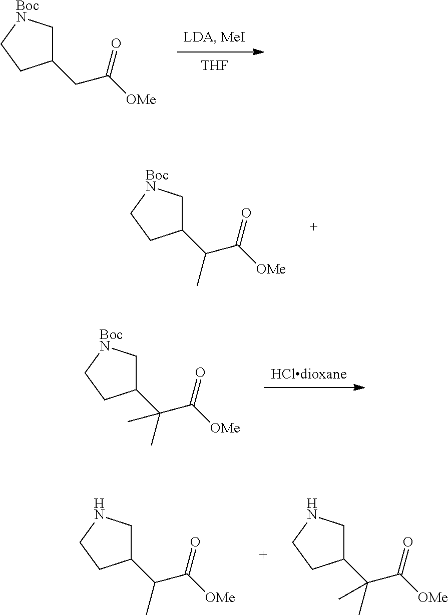

Step 1. To a stirred solution of tert-butyl 3-(2-methoxy-2-oxo-ethyl)pyrrolidine-1-carboxylate (400 mg, 1.64 mmol) in THF (5 mL), LDA (2 M, 3 mL) was added slowly at −78° C. and the mixture was stirred at −78° C. under N₂ for 1 hr. MeI (0.51 mL, 8.22 mmol) was then added to the reaction mixture, and the resulting mixture was stirred at 20° C. under N₂ for 1 hr. The reaction mixture was quenched with water (10 mL) and extracted with EtOAc (15 mL×2). The combined organic layers were washed with brine (20 mL), dried over anhydrous sodium sulfate, filtered and the filtrate was concentrated to afford a residue. The crude product was purified by flash chromatography on silica gel (0-100% of EtOAc in PE) to afford a mixture of tert-butyl 3-(2-methoxy-1,1-dimethyl-2-oxo-ethyl)pyrrolidine-1-carboxylate and tert-butyl 3-(2-methoxy-1-methyl-2-oxo-ethyl)pyrrolidine-1-carboxylate (130 mg, 30.7% yield) as a yellow oil.

Step 2. To a mixture of tert-butyl 3-(2-methoxy-1,1-dimethyl-2-oxo-ethyl)pyrrolidine-1-carboxylate and tert-butyl 3-(2-methoxy-1-methyl-2-oxo-ethyl)pyrrolidine-1-carboxylate (65.0 mg, 0.253 mmol) in 1,4-dioxane (1 mL), HCl/dioxane (3 mL) was added and the mixture was stirred at 25° C. for 16 hrs. The mixture was concentrated to give a mixture of methyl 2-methyl-2-pyrrolidin-3-yl-propanoate and methyl 2-pyrrolidin-3-ylpropanoate (40 mg, crude) as a yellow solid.

### Synthesis of 2-(1-(S-(2-(2-aminopyridin-3-yl)-5-phenyl-3H-imidazo[4,5-b]pyridin-3-yl)pyridin-2-yl)pyrrolidin-3-yl)-2-methylpropanoic acid (Compound I-39)

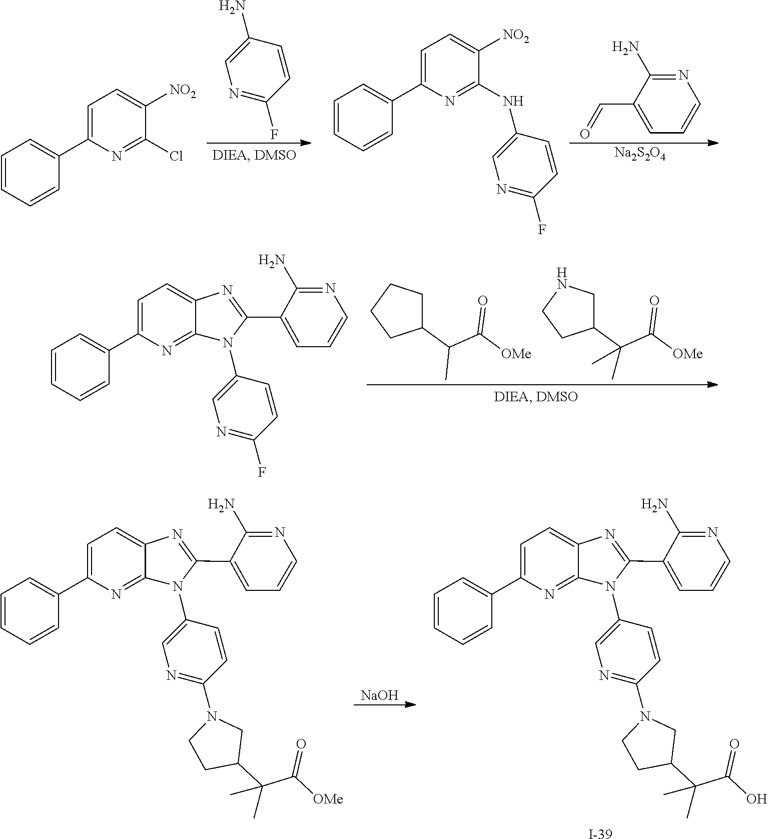

Step 1. To a stirred solution of 2-chloro-3-nitro-6-phenyl-pyridine (2.0 g, 8.52 mmol) and 6-fluoropyridin-3-amine (1.91 g, 17.0 mmol) in DMSO (30 mL), DIEA (4.7 mL, 25.6 mmol) was added, and the reaction was stirred at 90° C. under N₂ for 16 hrs. The reaction mixture was diluted with EtOAc (100 mL), washed with water (50 mL) and brine (100 mL 2), dried over anhydrous sodium sulfate, filtered and the filtrate was concentrated to afford a residue. The crude product was purified by flash chromatography on silica gel (30-95% of EtOAc in PE) to afford N-(6-fluoro-3-pyridyl)-3-nitro-6-phenyl-pyridin-2-amine (800 mg, 30.3% yield) as a yellow solid ¹H NMR (400 MHz, CDCl₃) δ=10.18 (s, 1H) 8.71-8.53 (m, 2H), 8.30-8.13 (m, 1H), 8.06-7.91 (m, 2H), 7.56-7.45 (m, 3H), 7.38 (d, 0.1=8.8 Hz, 1H), 7.02 (dd, J1=3.2, 8.8 Hz, 1H).

Step 2. To a stirred solution of 2-aminopyridine-3-carbaldehyde (0.53 g, 4.35 mmol) and N-(6-fluoro-3-pyridyl)-3-nitro-6-phenyl-pyridin-2-amine (1.0 g, 3.22 mmol) in DMSO (20 mL) and methanol (1 mL), Na₂S₂O₄ (1.68 g, 9.67 mmol) was added and the reaction mixture was stirred at 90° C. under N₂ for 16 hrs. The reaction mixture was diluted with EtOAc (50 mL) and washed with brine (50 mL×2), dried over anhydrous sodium sulfate, filtered and the filtrate was concentrated to afford a residue. The crude product was purified by flash chromatography on silica gel (90% of EtOAc in PE) to afford 3-[3-(6-fluoro-3-pyridyl)-5-phenyl-imidazo[4,5-b]pyridin-2-yl]pyridin-2-amine (500 mg, 40.60/yield) as a yellow solid. ¹H NMR (400 MHz, CDCl₃) δ=844 (d, J=2.0 Hz, 1H), 8.21-8.08 (m, 2H), 8.06-7.97 (m, 2H), 7.91-7.80 (m, 2H), 7.54-7.36 (m, 3H), 7.12 (dt, J=2.4, 8.0 Hz, 2H), 654 (s, 2H), 6.48 (dd, J=4.8, 7.6 Hz, 1H).

Step 3. To a stirred solution of 3-(3-(6-fluoropyridin-3-yl)-5-phenyl-3H-imidazo[4,5-b]pyridin-2-yl)pyridin-2-amine (150 mg, 0.392 mmol) and a mixture of methyl 2-pyrrolidin-3-ylpropanoate and methyl 2-methyl-2-pyrrolidin-3-yl-propanoate (67.0 mg, 0.392 mmol) in DMSO (2.00 mL), DIEA (0.36 mL, 1.96 mmol) was added and the reaction mixture was stirred at 100° C. under N₂ for 16 hrs. Water (10 mL) was then added, and the whole extracted with EtOAc (20 mL×3). The organic phase was washed with brine (20 mL×3), dried with Na₂SO₄ and concentrated. The residue was purified by prep-HPLC (Xtimate C18 150*40 mm*5 μm, water(HCl)-ACN) to give methyl 2-(1-(5-(2-(2-aminopyridin-3-yl)-5-phenyl-3H-imidazo[4,5-b]pyridin-3-yl)pyridin-2-yl)pyrrolidin-3-yl)-2-methylpropanoate (70 mg, 33.4% yield) as a yellow solid. LCMS Rₜ=0.51 min in 1 min chromatography, Xtimate C18 2.1*30 mm,3 um, purity 94.8%, MS ESI calcd for 533.25 [M+H]⁺ 534.25, found 534.3.

Step 4. To a solution of methyl 2-[1-[5-[2-(2-amino-3-pyridyl)-5-phenyl-imidazo[4,5-b]pyridin-3-yl]-2-pyridyl]pyrrolidin-3-yl]-2-methyl-propanoate (30.0 mg, 0.0562 mmol) in THF (0.500 mL), aq. NaOH (4 M) was added and the mixture stirred at 40° C. for 16 hrs. NaOH (22.0 mg, 0.562 mmol) and MeOH (1.00 mL) were subsequently added, and the mixture was stirred at 50° C. for 16 hrs. The mixture was concentrated, and the residue was dissolved with DMSO (2 mL), adjusted to pH ~-5, and then purified by prep-HPLC (Xtimate C18 150*40 mm*5 μm, water (HCl)-ACN) to give 2-(1-(5-(2-(2-aminopyridin-3-yl)-5-phenyl-3H-imidazo[4,5-b]pyridin-3-yl)pyridin-2-yl)pyrrolidin-3-yl)-2-methylpropanoic acid I-39 (16.7 mg, 56.4% yield) as a yellow solid. ¹H NMR (DMSO-d₆ 400 MHz) δ_{H}=8.33 (d, J=8.4 Hz, 1H), 8.24 (s, 1H), 8.05 (br d, J=8.0 Hz, 5H), 7.85 (br d, J=9.2 Hz, 1H), 7.53-7.39 (m, 3H), 6.98-6.82 (m, 2H), 3.71-3.68 (m, 2H), 3.47-3.36 (m, 1H), 3.28 (br t, J=10.4 Hz, 1H), 2.65-2.60 (m, 1H), 2.13-1.98 (m, 1H), 1.91-1.73 (m, 1H), 1.17 (br d, J=4.0 Hz, 6H). HPLC Rₜ=8.549 min in 15 min chromatography, 0-60AB, purity 99.5%. LCMS Rₜ=2.446 min in 4 min chromatography, Xtimate C18 2.1*30 mm, 3 μm, purity 100%, MS ESI calcd. for 519.24 [M+H]⁺ 520.24, found 520.1.

### Example 40. Synthesis of(1r,4r)-4-((5-(2-(2-aminopyridin-3-yl)-5-(4-fluorophenyl)-3H-imidazo[4,5-b]pyridin-3-yl)pyridin-2-yl)carbamoyl)cyclohexane-1-carboxylic acid (Compound I-40)

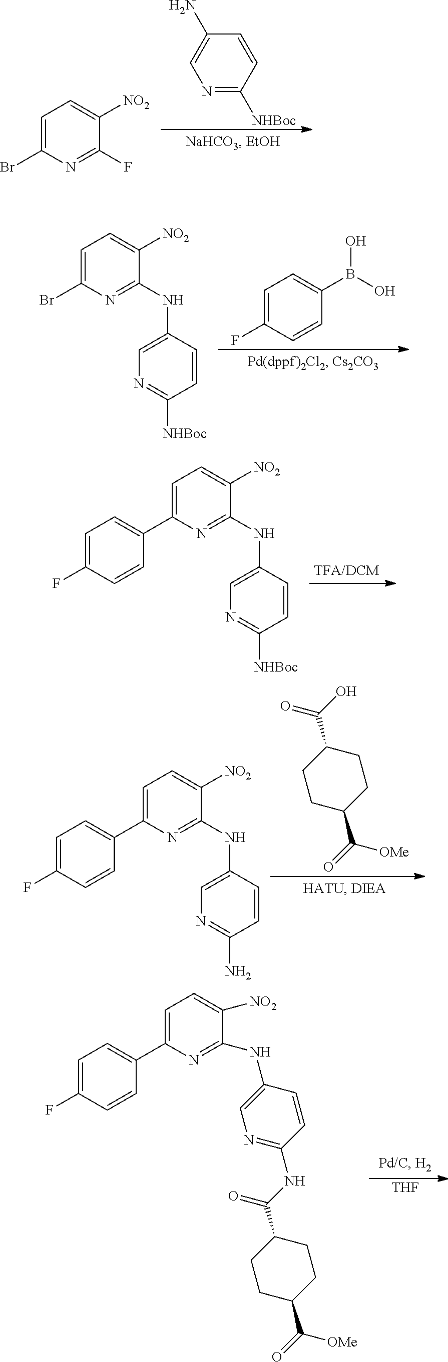

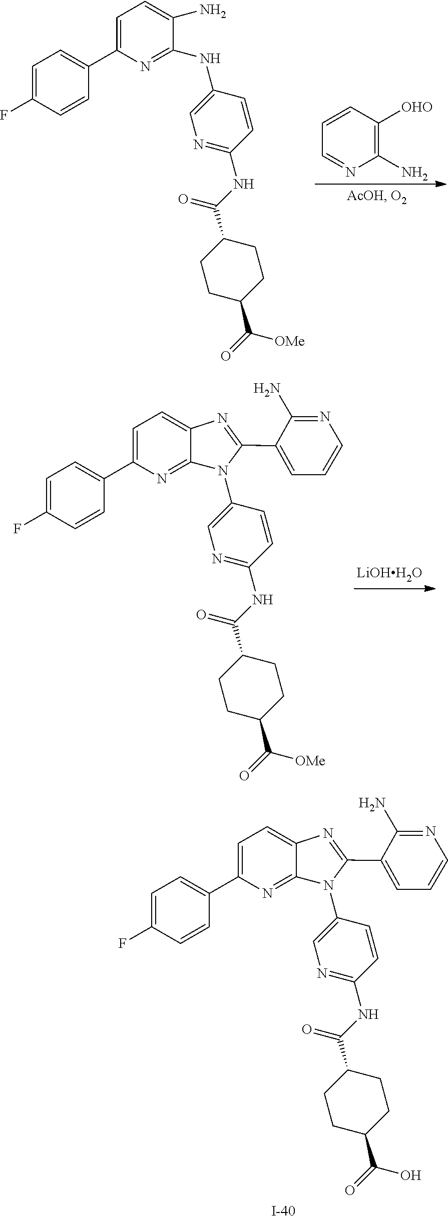

Step 1. To a solution of 6-bromo-2-fluoro-3-nitropyridine (1.00 g, 4.53 mmol) in ethanol (10 mL), NaHCO₃ (0.46 g, 4.53 mmol) and tert-butyl N-(5-amino-2-pyridyl) carbamate (0.85 g, 4.07 mmol) were added at 25° C. and the mixture was stirred for 16 hrs. Water (5 mL) was added to the reaction mixture and isopropyl ether (100 mL) was added to the mixture over 10 mins. The reaction mixture was filtered. The filter cake was concentrated to give tert-butyl (5-((6-bromo-3-nitropyridin-2-yl)amino)pyridin-2-yl)carbamate (1.80 g, 96.9% yield) as a yellow solid. ¹H NMR (DMSO-d₆, 400 MHz) δ_{H}=10.09 (s, 1H) 9.79 (s, 1H), 8.43-8.36 (m, 2H), 7.89-7.78 (m, 2H), 7.12 (d, J=8.4 Hz, NH), 1.48 (s, 9H).

Step 2. To a solution of tert-butyl (5-((6-bromo-3-nitropyridin-2-yl)amino)pyridin-2-yl)carbamate (1.80 g, 4.39 mmol) and (4-fluorophenyl) boronic acid (0.80 g, 5.70 mmol) in 1,4-dioxane (30 mL) and water (6 mL), Cs₂CO₃ (4.29 g, 13.2 mmol) and Pd(dppf)Cl₂ (0.32 g, 0.439 mmol) were added and the mixture was stirred at 100° C. for 12 hrs. The reaction mixture was concentrated water (5 mL) was added to the residue and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent of 0-15% EtOAc in PE) to give tert-butyl (5-((6-(4-fluorophenyl)-3-nitropyridin-2-yl)amino)pyridin-2-yl)carbamate (800 mg, 42.9% yield) as a yellow solid. ¹H NMR (DMSO-d₆, 400 MHz) δ_{H}=10.06 (s, 1H), 9.80 (s, 1H), 8.64-8.50 (m, 2H), 8.14-8.02 (m, 3H), 7.86 (d, J=9.2 Hz, 1H), 7.57 (d, J=8.8 Hz, 1H), 7.36 (m, 2H), 1.49 (s, 9H).

Step 3. To a solution of tert-butyl (5-((6-(4-fluorophenyl)-3-nitropyridin-2-yl)amino)pyridin-2-yl)carbamate (2.00 g, 4.70 mmol) in DCM (20 mL), TFA (2.0 mL, 4.70 mmol) was added and the mixture was stirred at 25° C. for 12 hrs. The reaction mixture was concentrated directly to give N-(6-(4-fluorophenyl)-3-nitropyridin-2-yl)pyridine-2,5-diamine (1.50 g, 91.2% yield) as a yellow oil, which was used in the next step directly. LCMS Rₜ=0.412 min in 1 min chromatography, purity 71.0%, MS ESI calcd. for 325.30, [M+H]⁺ 326.31, found 326.10.

Step 4. To a solution of N⁵-(6-(4-fluorophenyl)-3-nitropyridin-2-yl)pyridine-2,5-diamine (400 mg, 1.23 mmol) and trans-4-methoxycarbonylcyclohexanecarboxylic acid (229 mg, 1.23 mmol) in DMF (10 mL), HATU (701 mg, 1.84 mmol) and DIEA were added, and the mixture was stirred at 40° C. for 12 hrs. Water (5 mL) was added to the residue. The resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent of 0-12% MeOH in DCM) to give methyl (1r,4r)-4-((5-((6-(4-fluorophenyl)-3-nitropyridin-2-yl)amino)pyridin-2-yl)carbamoyl)cyclohexane-1-carboxylate (330 mg, 54.4% yield) as a light yellow oil. LCMS Rₜ=0.710 min in 1 min chromatography, purity 60%, MS ESI calcd. for 493.18, [M+H]⁺ 494.18, found 494.30.

Step 5. To a solution of methyl (1r,4r)-4-((5-((6-(4-fluorophenyl)-3-nitropyridin-2-yl)amino)pyridin-2-yl)carbamoyl)cyclohexane-1-carboxylate (330 mg, 0.669 mmol) in THF (10 mL), Pd/C (100 mg, purity 10%) was added under N₂. The reaction mixture was degassed and purged with H₂ (15 psi) (×3) and the mixture was stirred at 25° C. for 12 hrs under H₂ (15 psi). The reaction mixture was filtered and the filtrate was concentrated directly to give methyl (1r,4r)-4-((5-((3-amino-6-(4-fluorophenyl)pyridin-2-yl)amino)pyridin-2-yl)carbamoyl)cyclohexane-1-carboxylate (290 mg, 93.6% yield) as light yellow oil, which was used in the next step directly. ¹H NMR (DMSO-d₆, 400 MHz) δ_{H}=10.24 (s, 1H), 8.78 (s, 1H), 8.04-7.88 (m, 5H), 7.25-7.18 (m, 3H), 6.98 (d, J=8.0 Hz, 1H), 5.26 (s, 2H), 3.60 (s, 3H), 2.27-2.26 (m, 1H), 2.16 (s, 1H), 1.89 (s, 4H), 1.33 (s, 4H).

Step 6. To a solution of methyl (1r,4r)-4-((5-((3-amino-6-(4-fluorophenyl)pyridin-2-yl)amino)pyridin-2-yl)carbamoyl)cyclohexane-1-carboxylate (290 mg, 0.626 mmol) in acetic acid (4 mL), 2-aminopyridine-3-carbaldehyde (103 mg, 0.845 mmol) was added under O₂ and the mixture was stirred at 80° C. for 1 hr under O₂. The reaction mixture was concentrated directly. The residue was purified by flash silica gel chromatography (eluent of 0-5% MeOH in DCM) to give methyl (1r,4r)-4-((5-(2-(2-aminopyridin-3-yl)-5-(4-fluorophenyl)-3H-imidazo[4,5-b]pyridin-3-yl)pyridin-2-yl)carbamoyl)cyclohexane-1-carboxylate (260 mg, 73.5% yield) as a light yellow oil. ¹H NMR (DMSO-d₆, 400 MHz) δ_{H}=10.65 (s, 1H), 8.40 (d, J=2.4 Hz, 1H), 8.29-8.26 (m, 1H), 8.11-8.08 (m, 2H), 8.04-8.02 (m, 1H), 7.98-7.93 (m, 2H), 7.40-7.25 (m, 4H), 6.84 (s, 2H), 6.52-6.49 (m, 1H), 3.60 (s, 3H), 2.27 (s, 1H), 2.16 (s, 1H), 1.89-1.89 (m, 4H), 1.49-1.33 (m, 4H).

Step 7. To a solution of methyl (1r,4r)-4-((5-(2-(2-aminopyridin-3-yl)-5-(4-fluorophenyl)-3H-imidazo[4,5-b]pyridin-3-yl)pyridin-2-yl)carbamoyl)cyclohexane-1-carboxylate (260 mg, 0.460 mmol) in THF (3 mL) and water (3 mL), LiOH·H₂O (58 mg, 1.38 mmol) was added and the mixture was stirred at 25° C. for 2 hrs. The reaction mixture was concentrated directly. The residue was purified by prep-HPLC (Column: Welch Xtimate C18 150*30 mm*5 μm, mobile phase: [water (FA)-ACN]; B %: 0%-35%; 25 min) to give (1r,4r)-4-((5-(2-(2-aminopyridin-3-yl)-5-(4-fluorophenyl)-3H-imidazo[4,5-b]pyridin-3-yl)pyridin-2-yl)carbamoyl)cyclohexane-1-carboxylic acid 1-40 (23.0 mg, 8.97% yield) as a light yellow solid. ¹H NMR (DMSO-d₆, 400 MHz) δ_{H}=10.65 (s, 1H), 8.40 (d, J=2.4 Hz, 1H), 8.29-8.24 (m, 2H), 8.11-8.08 (m, 2H), 8.04-7.93 (m, 3H), 7.38-7.26 (m, 3H), 6.82 (s, 2H), 6.51 (dd, J=4.8, 7.6 Hz, 1H), 2.53-2.52 (m, 1H), 2.25-2.15 (m, 1H), 2.00-1.87 (m, 4H), 1.53-1.41 (m, 2H), 1.39-1.27 (m, 2H). HPLC Rₜ=2.602 min in 8 min chromatography, purity 98.9%. LCMS Rₜ=1.191 min in 2 min chromatography, purity 99.8%, MS ESI calcd. for 551.21, [M+H]⁺ 552.21, found 552.1.

### Example 41. Synthesis of (1r,4r)-4-((5-(2-(2-aminopyridin-3-yl)-5-(1H-pyrazol-1-yl)-3H-imidazo[4,5-b]pyridin-3-yl)pyridin-2-yl)carbamoyl)cyclohexane-1-carboxylic acid (Compound I-41)

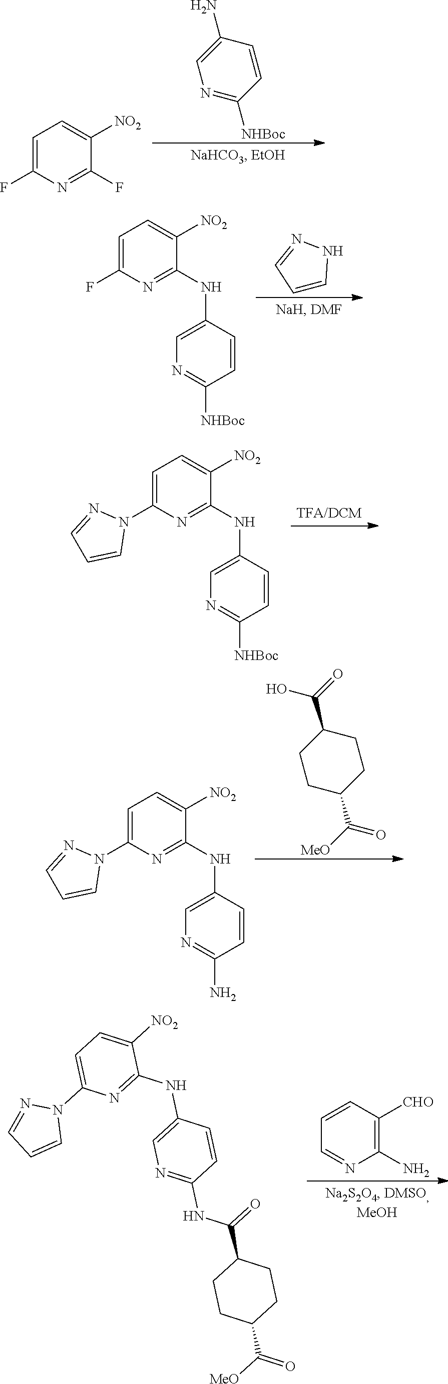

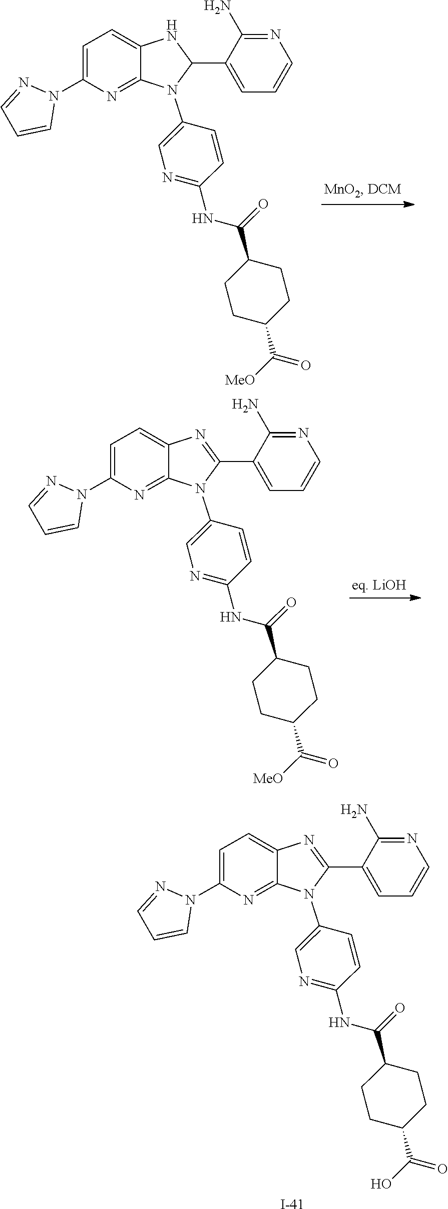

Step 1. To a solution of 2,6-difluoro-3-nitropyridine (5.00 g, 31.2 mmol) in ethanol (50 mL), NaHCO₃ (3.16 g, 31.2 mmol) and tert-butyl (5-aminopyridin-2-yl)carbamate (5.88 g, 28.1 mmol) were added at 25° C. and the mixture was stirred at 40° C. for 16 hrs. The reaction mixture was concentrated directly. Water (20 mL) was added to the residue and the resulting mixture was stirred at 25° C. for 2 hrs. The resulting mixture was filtered. The filter cake was stirred in ACN (20 mL) at 25° C. for 2 hrs. Then resulting mixture was filtered. The filter cake was concentrated to give tert-butyl (5-((6-fluoro-3-nitropyridin-2-yl)amino)pyridin-2-yl)carbamate (10.0 g, 71.0% yield) as a red solid. ¹H NMR (400 MHz, DMSO-d₆) δ_{H}=10.20 (s, 1H), 9.80 (s, 1H), 8.71 (dd, J=8.8, 7.6 Hz, 1H), 8.37 (d, J=2.4 Hz, 1H), 7.90-7.84 (m, 1H), 7.83-7.77 (m, 1H), 6.65 (dd, J=7.2, 3.6 Hz, 1H), 1.48 (s, 9H)

Step 2. To a solution of tert-butyl (5-((6-fluoro-3-nitropyridin-2-yl)amino)pyridin-2-yl)carbamate (2.00 g, 5.73 mmol) in DMF (3.00 mL), 1H-pyrazole (1.20 g, 17.2 mmol) and Cs₂CO₃ (3.70 g, 11.5 mmol) were added at 25° C. and the mixture was subjected to microwave irradiation at 80° C. for 1 hr. Water (10 mL) was added to the residue and the resulting mixture was extracted with EtOAc (10 mL×3). The combined organic phase was stirred at 25° C. for 2 hrs. The resulting mixture was filtered and the filter cake was dried to give tert-butyl (5-((3-nitro-6-(1H-pyrazol-1-yl)pyridin-2-yl)amino)pyridin-2-yl)carbamate (600 mg, 26.4% yield) as a red solid. ¹H NMR (400 MHz, DMSO-d₆) δ=9.77 (s, 1H), 8.69 (d, J=8.8 Hz, 1H), 8.45 (d, J=2.0 Hz, 1H), 8.18 (d, J=2.0 Hz, 1H), 8.03 (dd, J=8.4, 2.0 Hz, 1H), 7.96-7.84 (m, 3H), 7.38 (d, J=8.8 Hz, 1H), 6.64 (s, 1H), 1.49 (s, 9H).

Step 3. To a solution of tert-butyl (5-((3-nitro-6-(1H-pyrazol-1-yl)pyridin-2-yl)amino)pyridin-2-yl)carbamate (1.60 g, 3.89 mmol) in DCM (2 mL), TFA (0.5 mL) was added and the reaction stirred at 25° C. for 16 hrs. The reaction was concentrated directly. The resulting mixture was dissolved in EtOAc (10 mL) and the mixture adjusted to pH ~7 by aqueous NaOH (2 M). The resulting suspension was filtered. The filter cake was dried directly to afford N5-(3-nitro-6-(1H-pyrazol-1-yl)pyridin-2-yl)pyridine-2,5-diamine (580 mg, crude) as a black solid. ¹H NMR (400 MHz, DMSO-d₆) δ=10.09 (s, 1H), 8.68 (d, J=8.8 Hz, 1H), 8.18 (d, J=2.4 Hz, 1H), 8.12 (d, J=2.0 Hz, 1H), 7.93 (s, 1H), 7.81 (dd, J=8.8, 2.0 Hz, 1H), 7.37 (d, J=9.2 Hz, 1H), 6.79-6.58 (m, 4H).

Step 4. To a solution of compound N⁵-(3-nitro-6-(1H-pyrazol-1-yl)pyridin-2-yl)pyridine-2,5-diamine (500 mg, 1.68 mmol) in pyridine (8 mL), (1r,4r)-4-(methoxycarbonyl)cyclohexane-1-carboxylic acid (940 mg, 5.05 mmol) and EDCI (652 mg, 5.05 mmol) were added at 25° C. and the mixture was stirred at 80° C. for 2 hrs. Brine (30 mL) was added and the resulting mixture was extracted with EtOAc (20 mL×3). The combined organic phase was washed with brine (10 mL), water (10 mL), dried over anhydrous Na₂SO₄, filtered and concentrated. The residue was purified by flash silica gel chromatography (eluent of 0-13% MeOH in DCM) to give methyl (1r,4r)-4-((5-((3-nitro-6-(1H-pyrazol-1-yl)pyridin-2-yl)amino)pyridin-2-yl)carbamoyl)cyclohexane-1-carboxylate (230 mg, 29.4% yield) as a red solid. ¹H NMR (400 MHz, DMSO-d₆) δ=10.45 (s, 1H), 10.07 (s, 1H), 8.57 (d, J=8.8 Hz, 1H), 8.43 (d, J=2.4 Hz, 1H), 8.12 (d, J=8.8 Hz, 1H), 7.89 (dd, J=2.4, 8.8 Hz, 1H), 7.50 (d, J=2.0 Hz, 1H), 7.36 (d, J=8.8 Hz, 1H), 7.01 (d, J=2.0 Hz, 1H), 3.74 (s, 3H), 3.63-3.54 (m, 3H), 2.38-2.26 (m, 1H), 2.03-1.83 (m, 5H), 1.53-1.42 (m, 2H), 1.41-1.29 (m, 2H).

Step 5. To a solution of methyl (1r,4r)-4-((5-((3-nitro-6-(1H-pyrazol-1-yl)pyridin-2-yl)amino)pyridin-2-yl)carbamoyl)cyclohexane-1-carboxylate (230 mg, 0.494 mmol) in DMSO (2 mL) and methanol (2 mL), 2-aminonicotinaldehyde (121 mg, 0.988 mmol) and Na₂S₂O₄ (258 mg, 1.48 mmol) were added at 25° C. and the mixture was stirred at 100° C. for 16 hrs. The reaction was concentrated to remove MeOH. Water (10 mL) was then added to the residue and the resulting mixture was extracted with EtOAc (10 mL×3). The combined organic phase was washed with brine (5 mL), water (5 mL), dried over anhydrous Na₂SO₄, filter and concentrated. The residue was purified by flash silica gel chromatography (eluent of 0-65% EtOAc in PE) to give methyl (1r,4r)-4-((5-(2-(2-aminopyridin-3-yl)-5-(1H-pyrazol-1-yl)-1,2-dihydro-3H-imidazo[4,5-b]pyridin-3-yl)pyridin-2-yl)carbamoyl)cyclohexane-1-carboxylate (190 mg, 71.3% yield) as a yellow oil. LCMS Rₜ=0.740 min in 1.5 min chromatography, purity 40.4%, MS ESI calcd. for 539.24 [M+H]⁺ 540.24, found 540.4.

Step 6. To a solution of methyl (1r,4r)-4-((5-(2-(2-aminopyridin-3-yl)-5-(1H-pyrazol-1-yl)-1,2-dihydro-3H-imidazo[4,5-b]pyridin-3-yl)pyridin-2-yl)carbamoyl)cyclohexane-1-carboxylate (190 mg, 0.352 mmol) in DCM (5 mL), MnO₂ (612 mg, 7.04 mmol) was added at 25° C. and the mixture was stirred at 40° C. for 16 hrs. The reaction mixture was filtered. The filter cake was washed with DCM:MeOH (1:1, 50 mL). The filtrate was concentrated to give methyl (1r,4r)-4-((5-(2-(2-aminopyridin-3-yl)-5-(1H-pyrazol-1-yl)-3H-imidazo[4,5-b]pyridin-3-yl)pyridin-2-yl)carbamoyl) cyclohexane-1-carboxylate (190 mg, crude) as a yellow oil. LCMS Rₜ=1.179 min in 1.5 min chromatography, purity 86.3%, MS ESI calcd. for 537.22 [M+H]⁺ 538.22, found 538.1.

Step 7. To a solution of trans-methyl 4-[[5-[2-(2-amino-3-pyridyl)-5-pyrazol-1-yl-imidazo[4,5-b]pyridin-3-yl]-2-pyridyl]carbamoyl]cyclohexanecarboxylate (150 mg, 0.279 mmol) in THF (2 mL) and water (2 mL), LiOH·H₂O (80 mg, 1.95 mmol) was added at 25° C. and the mixture was stirred for 3 hrs. The reaction mixture was then adjusted to pH ~6 by aq. FA. The residue was purified by prep-HPLC (column: Welch Xtimate C18 40*200 mm 7 μm; condition: water(FA)-ACN; begin b: 0-40) to give (1r,4r)-4-((5-(2-(2-aminopyridin-3-yl)-5-(1H-pyrazol-1-yl)-3H-imidazo[4,5-b]pyridin-3-yl)pyridin-2-yl)carbamoyl)cyclohexane-1-carboxylic acid 1-41 (7.10 mg, 4.85% yield) as a white solid. ¹H NMR (400 MHz, DMSO-d₆) δ=10.65 (s, 1H), 8.44-8.33 (m, 3H), 8.26 (d, J=8.8 Hz, 1H), 8.03 (dd, J=4.8, 1.6 Hz, 1H), 8.00-7.94 (m, 2H), 7.81 (d, J=0.8 Hz, 1H), 7.36 (dd, J=7.6, 2.0 Hz, 1H), 6.77 (s, 2H), 6.56-6.47 (m, 2H), 2.53-2.52 (m, 1H), 2.25-2.15 (m, 1H), 2.01-1.86 (m, 4H), 1.53-1.41 (m, 2H), 1.39-1.27 (m, 2H). HPLC Rₜ=2.088 min in 8 min chromatography, purity 99.77%. LCMS Rₜ=0.579 min in 1.5 min chromatography, purity 96.64%, MS ESI calcd. for 523.21 [M+H]⁺ 524.21, found 524.2.

### Example 42. Synthesis of (1r,4r)-4-(((5-(2-(2-aminopyridin-3-yl)-5-phenyl-3H-imidazo[4,5-b]pyridin-3-yl)pyridin-2-yl)amino)methyl)cyclohexane-1-carboxylic acid (Compound I-43)

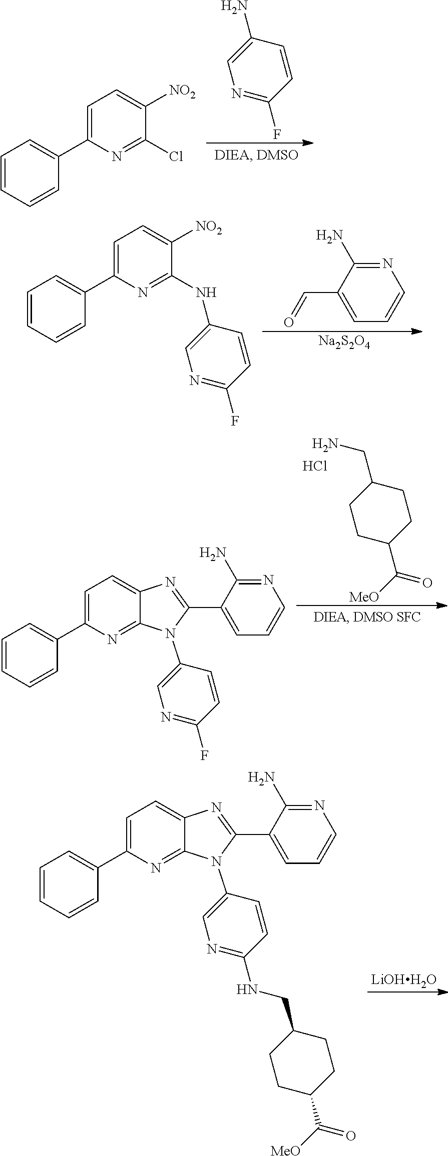

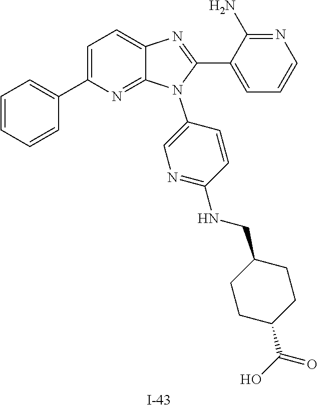

Step 1. To a stirred solution of 2-chloro-3-nitro-6-phenyl-pyridine (2.0 g, 8.52 mmol) and 6-fluoropyridin-3-amine (1.91 g, 17.0 mmol) in DMSO (30 mL), DIEA (4.7 mL, 25.6 mmol) was added and the reaction was stirred at 90° C. under N₂ for 16 hrs. The reaction mixture was diluted with EtOAc (100 mL), washed with water (50 mL) and brine (100 mL×2), dried over anhydrous sodium sulfate, filtered and the filtrate was concentrated to afford a residue. The crude product was purified by flash chromatography on silica gel (30-95% of EtOAc in PE) to afford N-(6-fluoro-3-pyridyl)-3-nitro-6-phenyl-pyridin-2-amine (800 mg, 30.3% yield) as a yellow solid. ¹H NMR (400 MHz, CDCl₃) δ=10.18 (s, 1H), 8.71-8.53 (m, 2H), 8.30-8.13 (m, 1H), 8.06-7.91 (m, 2H), 7.56-7.45 (m, 3H), 7.38 (d, J=8.8 Hz, 1H), 7.02 (dd, J=3.2, 8.8 Hz, 1H).

Step 2. To a stirred solution of 2-aminopyridine-3-carbaldehyde (0.53 g, 4.35 mmol) and N-(6-fluoro-3-pyridyl)-3-nitro-6-phenyl-pyridin-2-amine (1.0 g, 3.22 mmol) in DMSO (20 mL) and methanol (1 mL), Na₂S₂O₄ (1.68 g, 9.67 mmol) was added and the reaction mixture was stirred at 90° C. under N₂ for 16 hrs. The reaction mixture was diluted with EtOAc (50 mL) and washed with brine (50 mL×2), dried over anhydrous sodium sulfate, filtered and the filtrate was concentrated to afford a residue. The crude product was purified by flash chromatography on silica gel (90% of EtOAc in PE) to afford 3-[3-(6-fluoro-3-pyridyl)-5-phenyl-imidazo[4,5-b]pyridin-2-yl]pyridin-2-amine (500 mg, 40.6% yield) as a yellow solid. ¹H NMR (400 MHz, CDCl₃) δ=8.44 (d, J=2.0 Hz, 1H), 8.21-8.08 (m, 2H), 8.06-7.97 (m, 2H), 7.91-7.80 (m, 2H), 7.54-7.36 (m, 3H), 7.12 (dt, J=2.4, 8.0 Hz, 2H), 6.54 (s, 2H), 6.48 (dd, J=4.8, 7.6 Hz, 1H).

Step 3. To a solution of methyl 4-(aminomethyl)cyclohexanecarboxylate;hydrochloride (435 mg, 2.09 mmol) in DMSO (2.00 mL), DIEA (0.27 mL, 1.57 mmol) and 3-[3-(6-fluoro-3-pyridyl)-5-phenyl-imidazo[4,5-b]pyridin-2-yl]pyridin-2-amine (200 mg, 0.523 mmol) were added and the reaction stirred at 100° C. for 16 hrs. Water (20 mL) was added and the whole extracted with EtOAc (10 mL 3). The organic layer was washed with brine then dried over Na₂SO₄ and concentrated in vacuum to provide the crude material. The crude was purified by flash column (0-70% of EtOAc in PE). The material was purified by SFC (column: DAICEL CHIRALPAK AD 250 mm*30 mm, 10 μm; mobile phase: CO₂-i-PrOH; B %: 50%-50%, 150 min) to give methyl (1r,4r)-4-(((5-(2-(2-aminopyridin-3-yl)-5-phenyl-3H-imidazo[4,5-b]pyridin-3-yl)pyridin-2-yl)amino)methyl)cyclohexane-1-carboxylate (80.0 mg, 28.7% yield) as a red solid. ¹H NMR (400 MHz, DMSO-d₆) δ=8.24 (d, J=8.4 Hz, 1H), 8.06-8.00 (m, 4H), 7.96 (d, J=8.4 Hz, 1H), 7.51-7.44 (m, 3H), 7.43-7.36 (m, 2H), 6.98-6.90 (m, 3H), 6.60 (d, J=8.4 Hz, 1H), 6.49 (dd, J=4.8, 7.6 Hz, 1H), 3.59 (s, 3H), 3.14 (t, J=6.0 Hz, 2H), 2.35-2.20 (m, 1H), 1.99-1.80 (m, 4H), 1.60-1.47 (m, 1H), 1.40-1.25 (m, 2H), 1.06-1.00 (m, 2H).

Step 4. To a solution of methyl (1r,4r)-4-(((5-(2-(2-aminopyridin-3-yl)-5-phenyl-3H-imidazo[4,5-b]pyridin-3-yl)pyridin-2-yl)amino)methyl)cyclohexane-1-carboxylate (80 mg, 0.150 mmol) in THF (I mL) and water (1 mL), LiOH·H₂O (25 mg, 0.600 mmol) was added and the reaction was stirred at 25° C. for 16 hrs. The crude was purified by prep-HPLC (column: Welch Xtimate C18 150*30 mm*5 um; mobile phase: water (FA)-ACN; B %: 20%-60%, 30 min) to give (1r,4r)-4-(((5-(2-(2-aminopyridin-3-yl)-5-phenyl-3H-imidazo[4,5-b]pyridin-3-yl)pyridin-2-yl)amino)methyl)cyclohexane-1-carboxylic acid 1-43 (48.0 mg, 60.6% yield) as a yellow solid. LCMS Rₜ=2.684 min in 4 min chromatography, purity 99.3%, MS ESI calcd. For 519.24 [M+H]⁺ 520.24, found 520.1. HPLC Rₜ=4.269 min in 8 min chromatography, purity 99.3%. ¹H NMR (400 MHz, DMSO-d₆) δ=12.15-11.82 (m, 1H), 8.24 (d, J=8.4 Hz, 1H), 8.08-7.99 (m, 4H), 7.96 (d, J=8.4 Hz, 1H), 7.51-7.44 (m, 3H), 7.43-7.34 (m, 2H), 7.00-6.89 (m, 3H), 6.60 (d, J=8.8 Hz, 1H), 6.50 (dd, J=4.8, 7.6 Hz, 1H), 3.13 (t, J=6.4 Hz, 2H), 2.21-2.09 (m, 1H), 1.98-1.80 (m, 4H), 1.58-1.45 (m, 1H), 1.38-1.20 (m, 2H), 1.08-0.93 (m, 2H).

### Example 43. Synthesis of 6-[[4-[2-(2-amino-3-pyridyl)-5-phenyl-imidazo[4,5-b]pyridin-3-yl]phenyl]carbamoyl]spiro[3.3]heptane-2-carboxylic acid (Compound I-46)

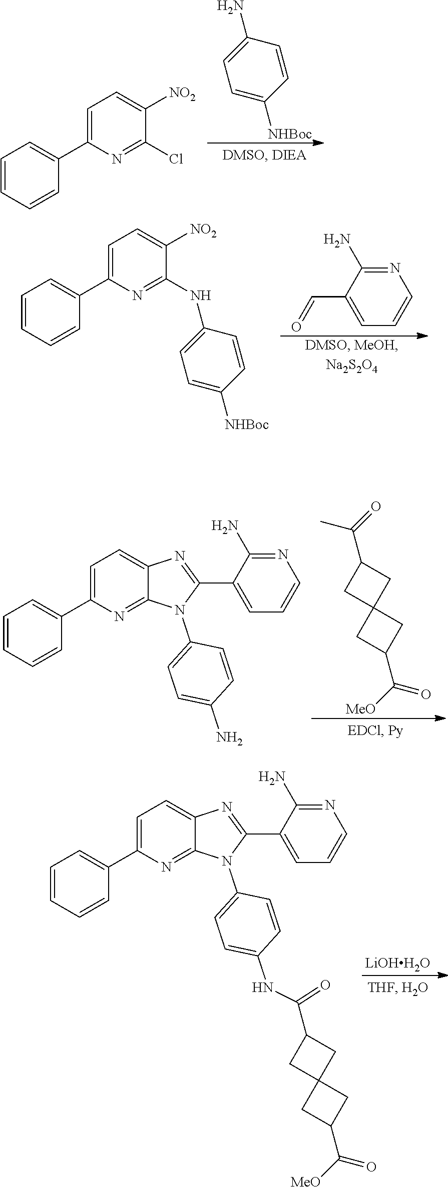

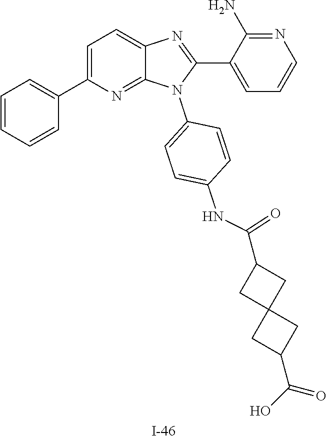

Step 1. To a stirred solution of 2-chloro-3-nitro-6-phenyl-pyridine (6.00 g, 25.6 mmol) and tert-butyl N-(4-aminophenyl)carbamate (7.00 g, 33.6 mmol) in DMSO (80 mL), DIEA (14 mL, 76.7 mmol) was added and the reaction mixture was stirred at 90° C. under N₂ for 12 hrs. The reaction was diluted with EtOAc (300 mL), washed with water (200 mL) and brine (200 mL×2), dried over anhydrous sodium sulfate, filtered and the filtrate was concentrated and purified by flash chromatography on silica gel (20-80% of EtOAc in PE) to give tert-butyl N-[4-[(3-nitro-6-phenyl-2-pyridyl)amino]phenyl]carbamate (8.00 g, 76.9% yield) as a yellow solid. ¹H NMR (400 MHz, DMSO-d₆) δ=10.05 (s, 1H), 9.39 (s, 1H), 8.59 (d, J=8.8 Hz, 1H), 8.20-8.00 (m, 2H), 7.70-7.44 (m, 8H), 1.49 (s, 9H).

Step 2. To a stirred solution of 2-aminopyridine-3-carbaldehyde (1.68 g, 13.8 mmol) and tert-butyl N-[4-[(3-nitro-6-phenyl-2-pyridyl)amino]phenyl]carbamate (4.00 g, 9.84 mmol) in DMSO (40 mL) and methanol (5 mL), Na₂S₂O₄ (6.85 g, 39.4 mmol) was added and the reaction mixture was stirred at 100° C. under N₂ for 16 hrs. The reaction mixture was diluted with EtOAc (500 mL), washed with water (300 mL) and brine (300 mL×2), dried over anhydrous sodium sulfate, filtered and the filtrate was concentrated to afford a residue. The crude product was purified by flash chromatography on silica gel (95% of EtOAc in PE) to afford 3-[3-(4-aminophenyl)-5-phenyl-imidazo[4,5-b]pyridin-2-yl]pyridin-2-amine (490 mg, 13.2% yield) as a yellow solid. ¹H NMR (400 MHz, DMSO-d₆) δ=8.22 (d, J=8.4 Hz, 1H), 8.09-7.90 (m, 4H), 7.50-7.43 (m, 2H), 7.42-7.35 (m, 1H), 7.28 (dd, J=2.0, 8.0 Hz, 1H), 7.16 (s, 2H), 7.11-7.04 (m, 2H), 6.74-6.60 (m, 2H), 6.43 (dd, J=4.8, 7.6 Hz, 1H), 5.50-5.38 (m, 2H).

Step 3. To a solution of 3-[3-(4-aminophenyl)-5-phenyl-imidazo[4,5-b]pyridin-2-yl]pyridin-2-amine (100 mg, 0.26 mmol) in pyridine (3 mL), 2-methoxycarbonylspiro[3.3]heptane-6-carboxylic acid (58 mg, 0.29 mmol) and EDCI (41 mg, 0.32 mmol) were added at 25° C. under N₂, and the mixture was stirred at 25° C. for 16 hrs. Water (20 mL) was added and the mixture was extracted with EtOAc (20 mL×3). The organic phase was washed with HCl (20 ml×2), brine (20 mL), dried over Na₂SO₄, filtered and concentrated under reduced pressure. The crude was purified by flash chromatography eluting with EtOAc in PE (from 0% to 100% of EtOAc in PE) to give methyl 6-[[4-[2-(2-amino-3-pyridyl)-5-phenyl-imidazo[4,5-b]pyridin-3-yl]phenyl]carbamoyl]spiro[3.3]heptane-2-carboxylate (145 mg, 98.2% yield) as a yellow oil. ¹H NMR (400 MHz, DMSO-d₆) δ=10.01 (s, 1H), 8.25 (d, J=8.8 Hz, 1H), 8.05-7.96 (m, 4H), 7.76 (d, J=8.8 Hz, 2H), 7.48-7.44 (m, 2H), 7.42-7.38 (m, 3H), 7.23 (dd, J=1.6, 7.6 Hz, 1H), 7.04 (s, 2H), 6.43 (dd, J=4.8, 7.6 Hz, 1H), 3.59 (s, 3H), 3.11 (t, J=8.4 Hz, 1H), 3.04 (t, 0.1=8.4 Hz, 1H), 2.34-2.25 (m, 4H), 2.21-2.11 (m, 4H).

Step 4. To a solution of methyl 6-[[4-[2-(2-amino-3-pyridyl)-5-phenyl-imidazo[4,5-b]pyridin-3-yl]phenyl]carbamoyl]spiro[3.3]heptane-2-carboxylate (145 mg, 0.26 mmol) in THF (1 mL) and water (1 mL), LiOH·H₂O (33 mg, 0.779 mmol) was added at 25° C. and the mixture was stirred for 3 hrs. The mixture was concentrated under reduced pressure to provide the crude material. The crude was purified by prep-HPLC (Welch Xtimate C18 150×30 mm×5 μm water (FA)-ACN) to give 6-[[4-[2-(2-amino-3-pyridyl)-5-phenyl-imidazo[4,5-b]pyridin-3-yl]phenyl]carbamoyl]spiro[3.3]heptane-2-carboxylic acid I-46 (78 mg, 53.9% yield) as a yellow solid. ¹H NMR (400 MHz, DMSO-d₆) δ=12 16-11.91 (m, 1H), 10.01 (s, 1H), 8.25 (d, J=8.8 Hz, 1H), 8.04-7.96 (m, 4H), 7.76 (d, J=8.8 Hz, 2H), 7.50-7.44 (m, 2H), 7.43-7.36 (m, 3H), 7.23 (dd, J=1.6, 7.6 Hz, 1H), 7.05 (br s, 2H), 6.43 (dd, J=4.8, 7.6 Hz, 1H), 3.11 (q, J=8.4 Hz, 1H), 2.98-2.88 (m, 1H), 2.33-2.10 (m, 8H). HPLC Rₜ=3.194 min in 8.0 min chromatography, 10-80AB_8 min.1cm, purity 98.3%. LCMS Rₜ=2.401 min in 4.0 min chromatography, purity 100%, MS ESI calcd. for 544.22 [M+H]⁺ 545.22, found 545.1.

### Example 44. Synthesis of 6-((4-(2-(2-aminopyridin-3-yl)-5-phenyl-3H-imidazo[4,5-b]pyridin-3-yl)-2-fluorophenyl)carbamoyl)spiro[3.3]heptane-2-carboxylic acid (Compound I-47)

### Synthesis of tert-butyl (4-amino-2-fluorophenyl)carbamate

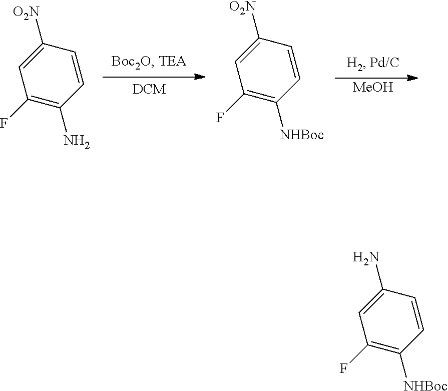 [SMILES: "MeOH" ; Nc1ccc([N+](=O)[O-])cc1F ; CC(C)(C)OC(=O)Nc1ccc([N+](=O)[O-])cc1F ; CC(C)(C)OC(=O)Nc1ccc(N)cc1F]

Step 1. To a solution of 2-fluoro-4-nitroaniline (5.00 g, 32.0 mmol) in DCM (50 mL), TEA (9.72 g, 96.1 mmol) and Boc₂0 (10.5 g, 48.0 mmol) were added at 25° C. and the mixture was stirred at 50° C. for 2 hrs. The mixture was diluted with DCM (100 mL) and washed with water (30 mL×2) The organic layer was dried over Na₂SO₄ and concentrated. The crude product was purified by column chromatography on silica gel (EtOAc in PE=0-5%) to afford tert-butyl (2-fluoro-4-nitrophenyl)carbamate (5.60 g, 68.2% yield) as a yellow oil. ¹H NMR (DMSO-d₆, 400 MHz) δ_{H}=9.73 (s, 1H), 8.15-8.03 (m, 3H), 1.48 (s, 9H).

Step 2. To a solution of tert-butyl (2-fluoro-4-nitrophenyl)carbamate (5.60 g, 21.9 mmol) in methanol (20 mL), Pd/C (2.00 g, 10% purity) was added under Ar atmosphere. The suspension was degassed and purged with H₂ (×3) and the reaction mixture was stirred under H₂ (15 psi) at 25° C. for 10 hrs. The reaction mixture was filtered via a celite pad, and the filter cake was washed with DCM:MeOH=10:1 (500 mL). The filtrate was concentrated to afford tert-butyl (4-amino-2-fluorophenyl)carbamate (3.80 g, 76.9% yield) as a yellow oil. ¹H NMR (DMSO-d₆, 400 MHz,) δ_{H}=8.28 (s, 1H), 6.95 (s, 1H), 6.37-6.25 (m, 2H), 5.22 (s, 2H), 1.48-1.32 (m, 9H).

### Synthesis of 6-((4-(2-(2-aminopyridin-3-yl)-5-phenyl-3H-imidazo[4,5-b]pyridin-3-yl)-2-fluorophenyl)carbamoyl)spiro[3.3]heptane-2-carboxylic acid (Compound I-47)

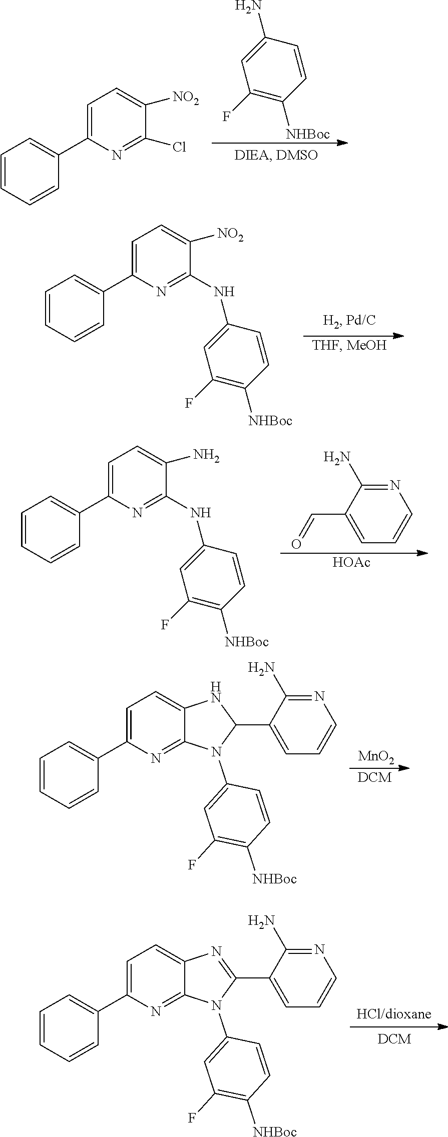

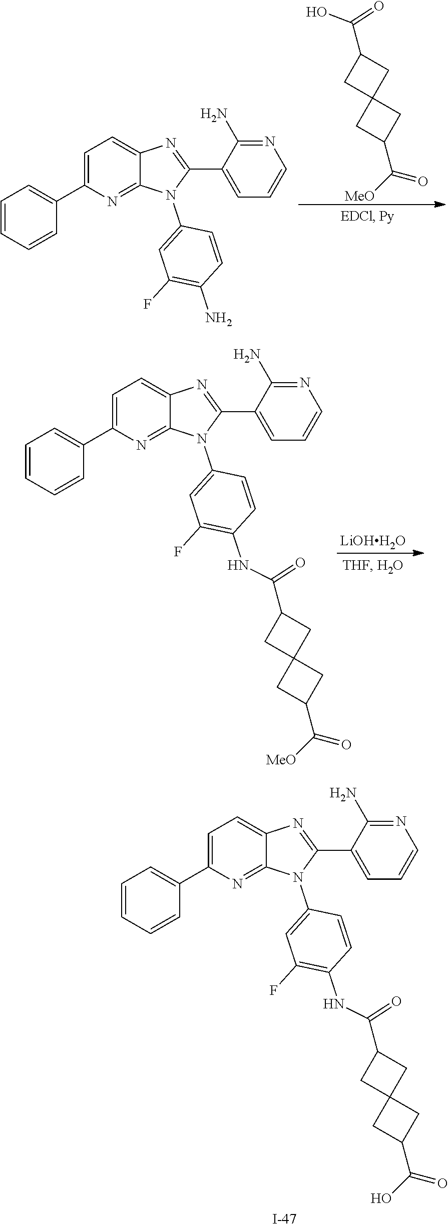

Step 1. To a solution of 2-chloro-3-nitro-6-phenylpyridine (2.30 g, 9.80 mmol) in DMSO (20 mL), tert-butyl (4-amino-2-fluorophenyl)carbamate (2.66 g, 11.8 mmol) and DIEA (3.80 g, 29.4 mmol) were added at 25° C. and the mixture was stirred at 105° C. for 14 hrs. EtOAc (60 mL) was added into the reaction and the resulting suspension was washed with brine (30 mL×3), dried over anhydrous Na₂SO₄, filtered and concentrated. The residue was purified by flash silica gel chromatography (ISCO®; 40 g, SepaFlash® Silica Flash Column, eluent of 0-10% EtOAc in PE @35 mL/min) to afford tert-butyl (2-fluoro-4-((3-nitro-6-phenylpyridin-2-yl)amino)phenyl)carbamate (1.50 g, 36.1% yield) as a red solid. ¹H NMR (DMSO-d₆, 400 MHz) δ_{H}=10.11 (s, 1H), 8.93 (s, 1H), 8.62 (d, J=8.8 Hz, 1H), 8.09 (dd, J=2.8, 6.4 Hz, 2H), 7.76 (dd, J=2.0, 13.2 Hz, 1H), 7.62 (d, J=8.8 Hz, 1H), 7.59-7.52 (m, 4H), 7.50-7.45 (m, 1H), 1.47 (s, 9H).

Step 2. To a solution of tert-butyl (2-fluoro-4-((3-nitro-6-phenylpyridin-2-yl)amino)phenyl)carbamate (1.00 g, 2.36 mmol) in methanol (10 mL) and THF (10 mL), Pd/C (500 mg, 10% purity) was added under Ar atmosphere. The suspension was degassed and purged with H₂ (×3) and the reaction mixture was stirred under H₂ (15 psi) at 25° C. for 12 hrs. The reaction mixture was filtered and the filter cake was washed with MeOH/DCM (1:1, 500 mL). The resulting suspension was concentrated directly to afford tert-butyl (4-((3-amino-6-phenylpyridin-2-yl)amino)-2-fluorophenyl)carbamate (865 mg, 93.1% yield) as a yellow solid. ¹H NMR (DMSO-d₆, 400 MHz) δ_{H}=8.64 (s, 1H), 8.10 (s, 1H), 7.91 (dd, J=1.2, 8.4 Hz, 3H), 7.44-7.32 (m, 4H), 7.27 (d, J=8.0 Hz, 2H), 6.99 (d, J=8.0 Hz, 1H), 5.32 (s, 2H), 1.45 (s, 9H).

Step 3. A solution of tert-butyl (4-((3-amino-6-phenylpyridin-2-yl)amino)-2-fluorophenyl)carbamate (865 mg, 2.19 mmol) and 2-aminopyridine-3-carbaldehyde (348 mg, 2.85 mmol) in acetic acid (5 mL) was stirred at 100° C. for 1 hr. The mixture was concentrated directly. The residue was purified by flash silica gel chromatography (ISCO®; 20 g, SepaFlash® Silica Flash Column, eluent of 0-50% EtOAc in PE @35 mL/min) to afford tert-butyl (4-(2-(2-aminopyridin-3-yl)-5-phenyl-1,2-dihydro-3H-imidazo[4,5-b]pyridin-3-yl)-2-fluorophenyl)carbamate (410 mg, 37.5% yield) as a yellow solid. ¹H NMR (DMSO-d₆, 400 MHz) δ=8.77 (s, 1H), 8.71 (s, 1H), 8.44 (s, 1H), 8.16-8.13 (m, 1H), 8.07 (d, J=7.6 Hz, 2H), 7.97-7.89 (m, 2H), 7.75-7.67 (m, 2H), 7.60 (d, J=8.0 Hz, 1H), 7.53-7.48 (m, 4H), 7.44-7.38 (m, 2H), 6.72 (dd, J=4.8, 7.2 Hz, 1H), 1.46 (s, 9H).

Step 4. To a solution of tert-butyl (4-(2-(2-aminopyridin-3-yl)-5-phenyl-1,2-dihydro-3H-imidazo[4,5-b]pyridin-3-yl)-2-fluorophenyl)carbamate (500 mg, 1.00 mmol) in DCM (10 mL), MnO₂ (872 mg, 10 mmol) was added at 25° C. and the mixture was stirred at 60° C. for 12 hrs. The reaction mixture was filtered and the filter cake was washed with DCM (20 mL) and then dried. The residue was purified by flash silica gel chromatography (ISCO®; 12 g, SepaFlash® Silica Flash Column, eluent of 0-50% EtOAc in PE @35 mL/min) to afford tert-butyl (4-(2-(2-aminopyridin-3-yl)-5-phenyl-3H-imidazo[4,5-b]pyridin-3-yl)-2-fluorophenyl)carbamate (400 mg, 74.2% yield) as a yellow solid. ¹H NMR (DMSO-d₆, 400 MHz) δ=9.23 (s, 1H), 8.27 (d, J=8.4 Hz, 1H), 8.09-7.95 (m, 4H), 7.80 (t, J=8.8 Hz, 1H), 7.54-7.44 (m, 3H), 7.43-7.37 (m, 1H), 7.32-7.22 (m, 2H), 6.91 (s, 2H), 6.48 (dd, J=4.8, 7.6 Hz, 1H), 1.49 (s, 9H).

Step 5. To a solution of tert-butyl (4-(2-(2-aminopyridin-3-yl)-5-phenyl-3H-imidazo[4,5-b]pyridin-3-yl)-2-fluorophenyl)carbamate (300 mg, 0.60 mmol) in DCM (3 mL), HCl/Dioxane (3.0 mL, 4M) was added and the mixture was stirred at 20° C. for 1 hr. The mixture was concentrated directly to afford 3-(3-(4-amino-3-fluorophenyl)-5-phenyl-3H-imidazo[4,5-b]pyridin-2-yl)pyridin-2-amine (300 mg, crude) as a yellow solid. ¹H NMR (DMSO-d₆, 400 MHz) δ=8.66-8.43 (m, 1H), 8.33 (d, 0.1=8.4 Hz, 1H), 8.13 (dd, J=1.6, 6.4 Hz, 1H), 8.08-8.01 (m, 3H), 7.92 (dd, J=1.6, 7.6 Hz, 1H), 7.53-7.38 (m, 4H), 7.34 (dd, J=2.4, 12.0 Hz, JH), 7.06 (dd, J=2.0, 8.8 Hz, 1H), 6.98-6.85 (m, 2H).

Step 6. To a solution of 3-(3-(4-amino-3-fluorophenyl)-5-phenyl-3H-imidazo[4,5-b]pyridin-2-yl)pyridin-2-amine (150 mg, 0.380 mmol) in pyridine (2 mL), 6-(methoxycarbonyl)spiro[3.3]heptane-2-carboxylic acid (75 mg, 0.38 mmol) and EDCI (218 mg, 1.14 mmol) were added at 25° C. and the mixture was stirred at 80° C. for 2 hrs. The mixture was diluted with EtOAc (8 mL), washed with saturated aqueous NaCl (4 mL×3). The combined organic phase was dried over anhydrous Na₂SO₄, filtered and concentrated. The residue was purified by Combi-Flash column (EtOAc in PE=0%-60/o) to afford methyl 6-((4-(2-(2-aminopyridin-3-yl)-5-phenyl-3H-imidazo[4,5-b]pyridin-3-yl)-2-fluorophenyl)carbamoyl)spiro[3.3]heptane-2-carboxylate (110 mg, 50.4% yield) as a yellow solid. ¹H NMR (DMSO-d₆, 400 MHz)⁶=9.74 (s, 1H), 8.27 (d, J=8.4 Hz, 1H), 8.09-7.98 (m, 5H), 7.55-7.45 (m, 3H), 7.43-7.37 (m, 1H), 7.32-7.24 (m, 2H), 6.91 (s, 2H), 6.48 (dd, J=4.8, 7.6 Hz, 1H), 3.59 (s, 3H), 3.25 (d, J=8.4 Hz, 1H), 3.03 (t, 0.1=8.4 Hz, 1H), 2.33-2.23 (m, 4H), 2.19-2.09 (m, 4H).

Step 7. A solution of methyl 6-((4-(2-(2-aminopyridin-3-yl)-5-phenyl-3H-imidazo[4,5-b]pyridin-3-yl)-2-fluorophenyl)carbamoyl)spiro[3.3]heptane-2-carboxylate (100 mg, 0.17 mmol) and LiOH·H₂O (22 mg, 0.520 mmol) in THF (2 mL) and water (2 mL) was stirred at 25° C. for 12 hrs. The mixture was filtered directly. The mixture was purified by prep-HPLC (column: Xtimate C18 200*40 mm*7 μm; mobile phase: [water (HCl)-ACN]; B %:10%-50%, 20.5 min) to afford 6-((4-(2-(2-aminopyridin-3-yl)-5-phenyl-3H-imidazo[4,5-b]pyridin-3-yl)-2-fluorophenyl)carbamoyl)spiro[3.3]heptane-2-carboxylic acid 1-47 (25.1 mg, 25.7% yield) as a yellow solid. ¹H NMR (DMSO-d₆, 400 MHz,) δ_{H}=8.27 (d, J=8.0 Hz, 1H), 8.09-7.87 (m, 5H), 7.83 (d, J=7.2 Hz, 1H), 7.58-7.32 (m, 4H), 7.25 (d, J=8.0 Hz, 1H), 6.84 (t, J=6.8 Hz, 1H), 3.25-3.11 (m, 1H), 2.98-2.84 (m, 1H), 2.34-2.00 (m, 8H). HPLC Rₜ=1.946 min in 8 min chromatography, purity 100%. LCMS Rₜ=1.846 min in 4 min chromatography, purity 100%, MS ESI calcd. for 562.21 [M+H]⁺ 567.21, found 563.2.

### Example 45. Synthesis of 3-((5-(2-(2-aminopyridin-3-yl)-5-phenyl-3H-imidazo[4,5-b]pyridin-3-yl)pyridin-2-yl)carbamoyl)bicyclo[3.1.0]hexane-6-carboxylic acid (Compound I-48)

### Synthesis of 6-(ethoxycarbonyl)bicyclo[3.1.0]hexane-3-carboxylic acid

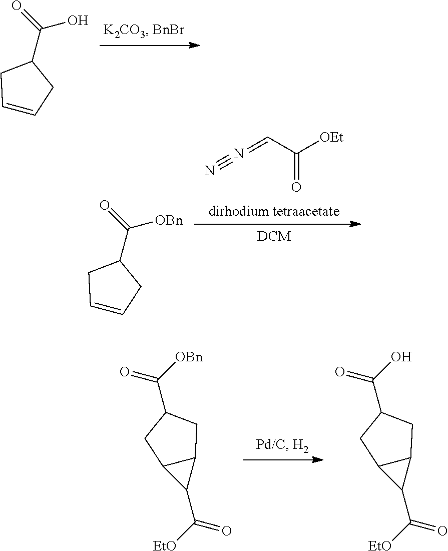 [SMILES: O=C(O)C1CC=CC1 ; CCOC(=O)C=[N+]=[N-] ; O=C(OCc1ccccc1)C1CC=CC1 ; CCOC(=O)C1C2CC(C(=O)OCc3ccccc3)CC21 ; CCOC(=O)C1C2CC(C(=O)O)CC21]

Step 1. To a mixture of cyclopent-3-ene-1-carboxylic acid (1.00 g, 8.92 mmol) and K₂CO₃ (2.47 g, 17.80 mmol) in acetone (10 mL), BnBr (1.68 g, 9.81 mmol) was added at 25° C. and the mixture was stirred at 60° C. for 2 hrs. The resulting mixture was filtered and the filtrate was concentrated. The crude product was purified by column chromatography on silica gel (100% PE) to give benzyl cyclopent-3-ene-1-carboxylate (1.40 g, 77.6% yield) as a colorless oil. ¹H NMR (400 MHz, CDCl₃) δ=7.41-7.32 (m, 5H), 5.66 (s, 2H), 5.11 (s, 2H), 3.23-3.15 (m, 1H), 2.67-2.53 (m, 4H).

Step 2. Ethyl 2-diazoacetate (2.71 g, 23.7 mmol) in D)CM (27 mL) was added slowly to a solution of benzyl cyclopent-3-ene-1-carboxylate (200 mg, 0.99 mmol) and dirhodium tetraacetate (87 mg, 0.20 mmol) in DCM (13 mL) at 25° C. After addition, the mixture was stirred at 25° C. for 10 hrs. The reaction mixture was filtered via a celite pad and the filtrate was concentrated. The crude product was purified by column chromatography on silica gel (EtOAc in PE, 3%) to give 3-benzyl 6-ethyl bicyclo[3.1.0]hexane-3,6-dicarboxylate (2.50 g, 43.8% yield) as a colourless oil. LCMS Rₜ=0.951 min in 1.5 min chromatography, Agilent PoroShell 120 EC-C18 2.7 μm 3.0*50 mm, purity 63.5%, MS ESI calcd. for 288.34 [M+H]⁺ 289.34, found 289.3.

Step 3. To a solution of 3-benzyl 6-ethyl bicyclo[3.1.0]hexane-3,6-dicarboxylate (1.00 g, 3.47 mmol) in methanol (10 mL), wet Pd/C (300 mg, 10%) was added under N₂ atmosphere. The suspension was degassed and purged with H₂ (×3) and the reaction mixture was stirred under H₂ (50 psi) at 25° C. for 10 hrs. The mixture was filtered via a celite pad and the filter cake was washed with DCM:MeOH=1.1 (300 mL). The organic phase was concentrated to give 6-(ethoxycarbonyl)bicyclo[3.1.0]hexane-3-carboxylic acid (740 mg, crude) as a white solid, which was used in the next step directly. LCMS Rₜ=1.077 min in 2.5 min chromatography, Agilent PoroShell 120 EC-C18 2.7 μm 3.0*50 mm, purity 20.7%, MS ESI calcd. for 198.22 [M+H]⁺ 199.22, found 199.1.

### Synthesis of 3-((S-(2-(2-aminopyridin-3-yl)-5-phenyl-3H-imidazo[4,5-b]pyridin-3-yl)pyridin-2-yl)carbamoyl)bicyclo[3.1.0]hexane-6-carbocylic acid (Compound I-48)

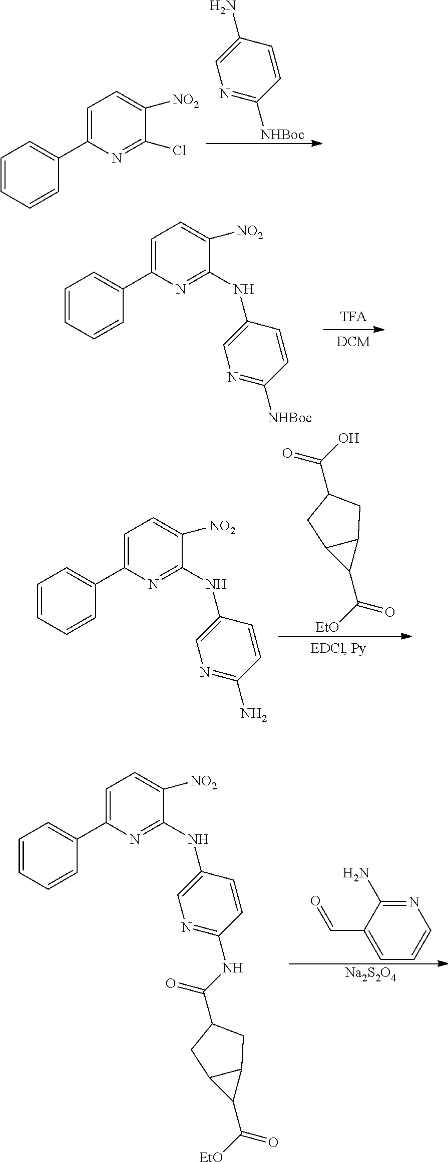

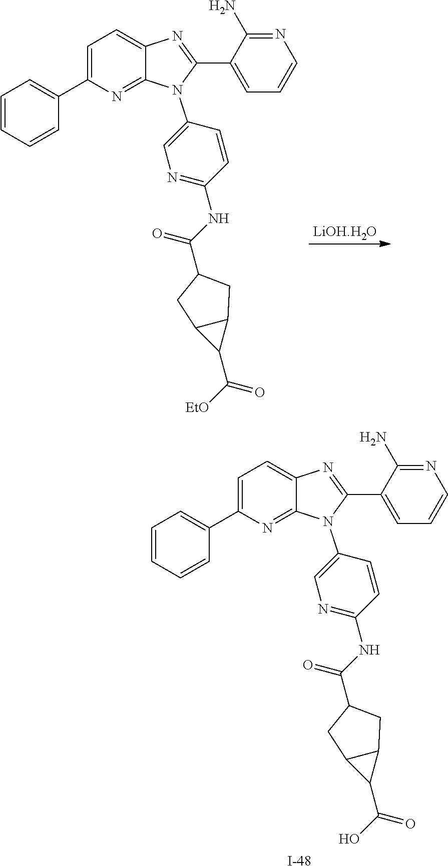

Step 1. To a solution of 2-chloro-3-nitro-6-phenylpyridine (1.70 g, 7.25 mmol) in DMSO (15 mL), tert-butyl (5-aminopyridin-2-yl)carbamate (1.82 g, 8.69 mmol) and DIEA (2.81 g, 21.7 mmol) were added at 25° C. and the mixture was stirred at 80° C. for 14 hrs. EtOAc (20 mL) was added to the reaction and the resulting suspension was washed with saturated aqueous LiCl (20 mL×3), dried over anhydrous Na₂SO₄, filtered and concentrated. Isopropyl ether (20 mL) was added, and the mixture stirred for 10 mins. The reaction mixture was filtered and the filter cake was concentrated to give tert-butyl (5-((3-nitro-6-phenylpyridin-2-yl)amino)pyridin-2-yl)carbamate (3.80 g, crude) as a red solid. ¹H NMR (CDCl₃, 400 MHz) δ_{H}=10.18 (s, 1H), 8.65-8.55 (m, 2H), 8.18 (d, J=9.2 Hz, 1H), 8.09-7.98 (m, 3H), 7.66-7.58 (m, 1H), 7.52 (s, 3H), 7.36 (d, J=8.4 Hz, 1H), 1.57 (s, 9H).

Step 2. To a solution of tert-butyl (5-((3-nitro-6-phenylpyridin-2-yl)amino)pyridin-2-yl)carbamate (12.4 g, 30.4 mmol) in DCM (60 mL), TFA (60 mL, 30.4 mmol) was added and the mixture was stirred at 25° C. for 10 hrs. The reaction mixture was concentrated and the mixture was adjusted to pH ~7 by NaOH (2 M). The resulting mixture was extracted with EtOAc (100 mL×3). The combined organic phase was dried over anhydrous Na₂SO₄, filtered and concentrated to give N5-(3-nitro-6-phenylpyridin-2-yl)pyridine-2,5-diamine (11.80 g, crude) as a white solid, which was used in the next step directly. ¹H NMR (DMSO-d₆ 400 MHz) δ_{H}=10.00 (s, 1H), 8.61 (d, J=8.8 Hz, 1H), 8.29 (d, J=2.4 Hz, 1H), 8.19 (dd, J=2.4, 9.2 Hz, 1H), 8.06-7.99 (m, 2H), 7.60 (d, J=8.8 Hz, 1H), 7.54-7.46 (m, 3H), 7.03 (d, J=9.6 Hz, 1H).

Step 3. To a solution of N⁵-(3-nitro-6-phenylpyridin-2-yl)pyridine-2,5-diamine (1.00 g, 3.25 mmol) and 6-(ethoxycarbonyl)bicyclo[3.1.0]hexane-3-carboxylic acid (774 mg, 3.90 mmol) in pyridine (10 mL), EDCI (1.87 g, 9.76 mmol) was added at 25° C. and the mixture was stirred at 80° C. for 2 hrs. The mixture was diluted with EtOAc (60 mL) and washed with brine (30 mL×2). The organic layer was dried over Na₂SO₄ and concentrated to give crude product. The crude product was purified by column chromatography on silica gel (EtOAc in PE, 15%) to give ethyl 3-((5-((3-nitro-6-phenylpyridin-2-yl)amino)pyridin-2-yl)carbamoyl)bicyclo[3.1.0]hexane-6-carboxylate (440 mg, 27.7% yield) as a white solid. LCMS Rₜ=0.953 min in 1.5 min chromatography, Agilent PoroShell 120 EC-C18 2.7 μm 3.0*50 mm, purity 93.9%, MS ESI calcd. For 487.19 [M+H]⁺ 488.19, found 488.5.

Step 4. To a solution of ethyl 3-((5-((3-nitro-6-phenylpyridin-2-yl)amino)pyridin-2-yl)carbamoyl)bicyclo[3.1.0]hexane-6-carboxylate (440 mg, 0.90 mmol) in DMSO (10 mL) and methanol (2 mL), 2-aminonicotinaldehyde (220 mg, 1.81 mmol) and Na₂S₂O₄ (943 mg, 5.42 mmol) were added at 25° C. and the mixture was stirred at 100° C. for 10 hr. The reaction was quenched with saturated aqueous NaHCO₃ (30 mL), dropwise, and the resulting mixture was extracted with EtOAc (50 mL×3). The combined organic phase was washed with brine (80 mL), dried over anhydrous Na₂SO₄, filtered and concentrated. The crude product was purified by column chromatography on silica gel (EtOAc in PE, 43%) to give ethyl 3-((5-(2-(2-aminopyridin-3-yl)-5-phenyl-3H-imidazo[4,5-b]pyridin-3-yl)pyridin-2-yl)carbamoyl)bicyclo[3.1.0]hexane-6-carboxylate (280 mg, 55.4% yield) as a yellow oil. LCMS Rₜ=1.431 min in 2 min chromatography, Agilent PoroShell 120 EC-C18 2.7 μm 3.0*50 mm, purity 86.2%, MS ESI calcd. For 559.23 [M+H]⁺ 560.23. found 560.2.

Step 5. To a solution of ethyl 3-((5-(2-(2-aminopyridin-3-yl)-5-phenyl-3H-imidazo[4,5-b]pyridin-3-yl)pyridin-2-yl)carbamoyl)bicyclo[3.1.0]hexane-6-carboxylate (280 mg, 0.50 mmol) in water (2 mL) and THF (2 mL), hydroxylithium;hydrate (84.0 mg, 2.00 mmol) was added at 25° C. and the mixture was stirred for 3 hrs. The resulting mixture was extracted with DCM (30 mL 3). The combined organic phase was washed with brine (50 mL), dried over anhydrous Na₂SO₄, filtered and concentrated. The residue was purified by prep-HPLC (Column: Welch Xtimate C₁₈ 40*200 mm 7 μm, mobile phase. [water (HCl)-ACN]; B %: 8%-48%; 20 min) to give 3-((5-(2-(2-aminopyridin-3-yl)-5-phenyl-3H-imidazo[4,5-b]pyridin-3-yl)pyridin-2-yl)carbamoyl)bicyclo[3.1.0]hexane-6-carboxylic acid 1-48 (90.0 mg, 44.4% yield) as a yellow solid. ¹H NMR (DMSO-d₆, 400 MHz) δ_{H}=10.71 (s, 1H), 8.53 (d, J=2.4 Hz, 1H), 8.37 (d, J=8.8 Hz, 1H), 8.27 (d, J=8.8 Hz, 1H), 8.15 (dd, J=1.2, 6.0 Hz, 1H), 8.11-8.05 (m, 3H), 8.05-8.01 (m, 1H), 8.01-7.97 (m, 1H), 7.52-7.46 (m, 2H), 7.45-7.40 (m, 1H), 6.99-6.93 (m, 1H), 2.76-2.68 (m, 1H), 2.16-2.08 (m, 2H), 2.07-1.96 (m, 2H), 1.85-1.79 (m, 2H), 1.31 (t, J=2.8 Hz, 1H). HPLC Rₜ=1.862 min in 4 min chromatography, 10-80 AB, purity 98.4%. LCMS Rₜ=1.712 min in 4 min chromatography, Agilent PoroShell 120 EC-C18 2.7 μm 3.0*50 mm, purity 97.4%, MS ESI calcd. for 531.20 [M+H]⁺ 532.20, found 532.2.

### Example 46. Synthesis of 1-[[4-[2-(2-amino-3-pyridyl)-5-phenyl-imidazo[4,5-b]pyridin-3-yl]phenyl]carbamoyl]pyrrolidine-3-carboxylic acid (Compound I-52)

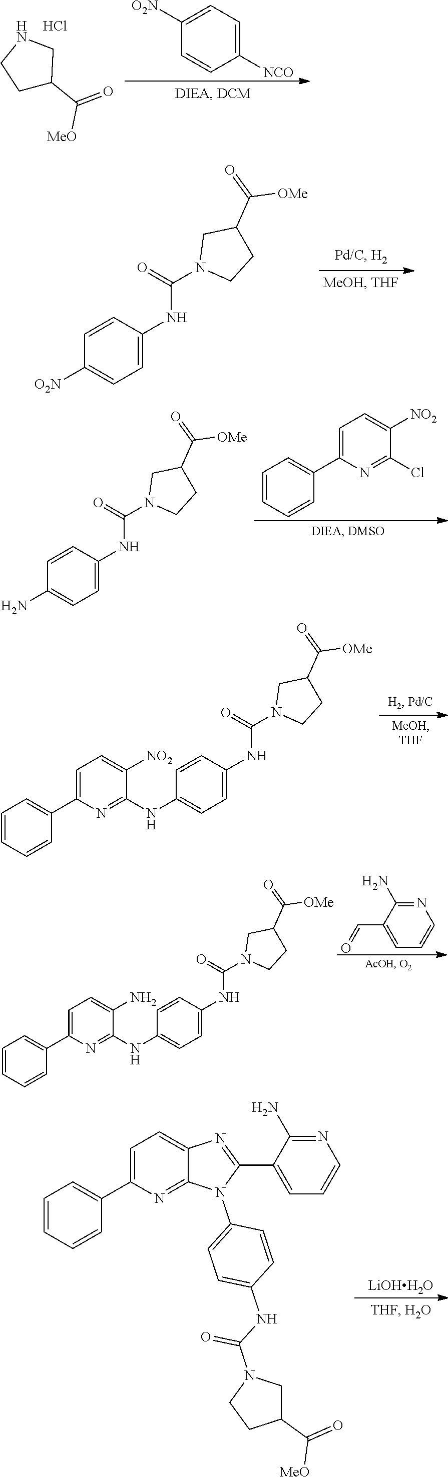

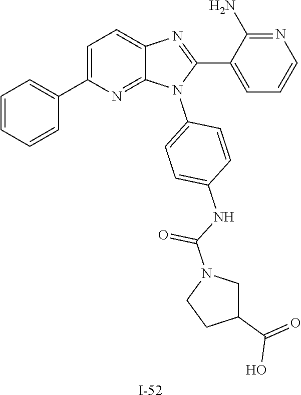

Step 1. To a stirred solution of methyl pyrrolidine-3-carboxylate (2.00 g, 15.5 mmol) in DCM (30 mL), 1-isocyanato-4-nitro-benzene (2.54 g, 15.5 mmol) and DIEA (9.98 g, 77.4 mmol) were added dropwise at 0° C. and the reaction mixture was stirred at 25° C. under N₂ for 16 hrs. The reaction mixture was concentrated and purified by flash chromatography on silica gel (10% methanol in DCM) to afford methyl 1-[(4-nitrophenyl)carbamoyl]pyrrolidine-3-carboxylate (1.20 g, 26.4% yield) as a yellow solid. ¹H NMR (400 MHz, DMSO-d₆) δ=8.92 (s, 1H), 8.21-8.09 (m, 2H), 7.85-7.73 (m, 2H), 3.74-3.39 (m, 7H), 3.28-3.19 (m, 1H), 2.22-2.00 (m, 2H).

Step 2. To a stirred solution of methyl 1-[(4-nitrophenyl)carbamoyl]pyrrolidine-3-carboxylate (1.20 g, 4.09 mmol) in methanol (25 mL) and THF (25 mL), Pd/C (200 mg, 50% wet by water, 10% Pd) was added under argon. The reaction was degassed and purged with argon (×3), then degassed and purged with H₂ (×3), and stirred under H₂ (50 psi) for 10 hrs at 50° C. Upon completion, the mixture was filtered under argon to afford methyl 1-[(4-aminophenyl)carbamoyl]pyrrolidine-3-carboxylate (1.00 g, 92.8% yield) as a yellow oil. ¹H NMR (400 MHz, DMSO-d₆) δ=7.74 (s, 1H), 7.14-6.95 (m, 2H), 6.57-6.35 (m, 2H), 4.70 (s, 2H), 3.65 (s, 3H), 3.61-3.35 (m, 4H), 3.23-3.17 (m, 1H), 2.19-1.95 (m, 2H).

Step 3. To a stirred solution of 2-chloro-3-nitro-6-phenyl-pyridine (891 mg, 3.80 mmol) and methyl 1-[(4-aminophenyl)carbamoyl]pyrrolidine-3-carboxylate (1.00 g, 3.80 mmol) in DMSO (15 mL), DIEA (3.5 mL, 19.0 mmol) was added and the reaction mixture was stirred at 85° C. under N₂ for 16 hrs. The reaction mixture was diluted with EtOAc (50 mL), washed with water (50 mL) and brine (50 mL×2), dried over anhydrous sodium sulfate, filtered and the filtrate was concentrated and purified by flash chromatography on silica gel (60-80% of EtOAc in PE) to afford methyl 1-[[4-[(3-nitro-6-phenyl-2-pyridyl)amino]phenyl]carbamoyl]pyrrolidine-3-carboxylate (1.00 g, 57.1% yield) as a yellow oil. ¹H NMR (400 MHz, DMSO-d₆) δ=10.05 (s, 1H), 8.59 (d, J=8.8 Hz, 1H), 8.27 (s, 1H), 8.15-8.02 (m, 2H), 7.65-7.45 (m, 8H), 3.74-3.38 (m, 7H), 3.26-3.19 (m, 1H), 2.23-2.01 (m, 2H).

Step 4. To a stirred solution of methyl 1-[[4-[(3-nitro-6-phenyl-2-pyridyl)amino]phenyl]carbamoyl]pyrrolidine-3-carboxylate (1.00 g, 2.17 mmol) in methanol (10 mL) and THF (15 mL), Pd/C (200 mg, 50% wet by water, 10% Pd) was added under argon. The reaction was degassed and purged with argon (×3), then degassed and purged with H₂ (×3) and stirred under H₂ (50 psi) for 10 hrs at 50° C. Upon completion, the mixture was filtered under argon to afford methyl 1-[[4-[(3-amino-6-phenyl-2-pyridyl)amino]phenyl]carbamoyl]pyrrolidine-3-carboxylate (700 mg, 74.9% yield). ¹H NMR (400 MHz, DMSO-d₆) δ=8.05 (s, 1H), 7.96-7.86 (m, 2H), 7.72 (s, 1H), 7.67-7.59 (m, 2H), 7.45-7.34 (m, 4H), 7.27-7.16 (m, 2H), 6.94 (d, J=8.0 Hz, 1H), 5.22 (s, 2H), 3.69-3.39 (m, 7H), 3.26-3.19 (m, 1H), 2.21-2.01 (m, 2H).

Step 5. A mixture of methyl 1-[[4-[(3-amino-6-phenyl-2-pyridyl)amino]phenyl]carbamoyl]pyrrolidine-3-carboxylate (300 mg, 0.69 mmol) and 2-aminopyridine-3-carbaldehyde (127 mg, 1.04 mmol) in acetic acid (5 mL) was stirred at 80° C. under O₂ for 1 hr. The reaction was diluted with EtOAc (20 mL), washed with water (20 mL) and brine (20 mL×2), dried over anhydrous sodium sulfate, filtered and the filtrate was concentrated to afford a residue. The crude product was purified by flash chromatography on silica gel (95-100% EtOAc in PE) to afford methyl 1-[[4-[2-(2-amino-3-pyridyl)-5-phenyl-imidazo[4,5-b]pyridin-3-yl]phenyl]carbamoyl]pyrrolidine-3-carboxylate (150 mg, 40.4% yield) as a yellow solid. ¹H NMR (400 MHz, DMSO-d₆) δ=8.47 (s, 1H), 8.25 (d, J=8.4 Hz, 1H), 8.08-7.91 (m, 4H), 7.70 (d, J=9.2 Hz, 2H), 7.53-7.31 (m, 5H), 7.24 (dd, J=2.0, 7.6 Hz, 1H), 7.09 (s, 2H), 6.43 (dd, J=4.8, 8.0 Hz, 1H), 3.73-3.41 (m, 7H), 3.28-3.21 (m, 1H), 2.23-2.03 (m, 2H).

Step 6. To a stirred solution of methyl 1-[[4-[2-(2-amino-3-pyridyl)-5-phenyl-imidazo[4,5-b]pyridin-3-yl]phenyl]carbamoyl]pyrrolidine-3-carboxylate (150 mg, 0.28 mmol) in THF (2 mL) and water (2 mL), LiOH·H₂O (34 mg, 0.84 mmol) was added and the reaction mixture was stirred at 25° C. under N₂ for 2 hrs. The reaction mixture was concentrated and pH adjusted to pH ~5 with 1N HCl and purified by prep-HPLC (column: Welch Xtimate C18 150×30 mm×5 mm, method: water (FA)-ACN, begin B: 17, end B: 47) to afford 1-[[4-[2-(2-amino-3-pyridyl)-5-phenyl-imidazo[4,5-b]pyridin-3-yl]phenyl]carbamoyl]pyrrolidine-3-carboxylic acid 1-52 (54 mg, 37.1% yield) as a white solid. ¹H NMR (400 MHz, DMSO-d₆) δ=12.65 (s, 1H), 8.45 (s, 1H), 8.25 (d, J=8.4 Hz, 1H), 8.09-7.90 (m, 4H), 7.70 (d, J=8.8 Hz, 21H), 7.51-7.31 (m, 5H), 7.24 (dd, J=2.0, 7.6 Hz, 1H), 7.09 (s, 2H), 6.43 (dd, J=4.8, 7.6 Hz, 1H), 3.68-3.57 (m, 2H), 3.54-3.43 (m, 2H), 3.17-3.06 (m, 1H), 2.22-1.99 (m, 2H). HPLC Rₜ=2.689 min in 8 min chromatography, purity 98.91%. LCMS Rₜ=1.634 min in 5 min chromatography, purity 97.64%, MS ESI calcd. for 519.20, [M+H]⁺ 520.20, found 520.6.

### Example 47. Synthesis of 1-[[4-[2-(2-amino-3-pyridyl)-5-phenyl-imidazo[4,5-b]pyridin-3-yl]phenyl]carbamoyl]piperidine-4-carboxylic acid (Compound I-53)

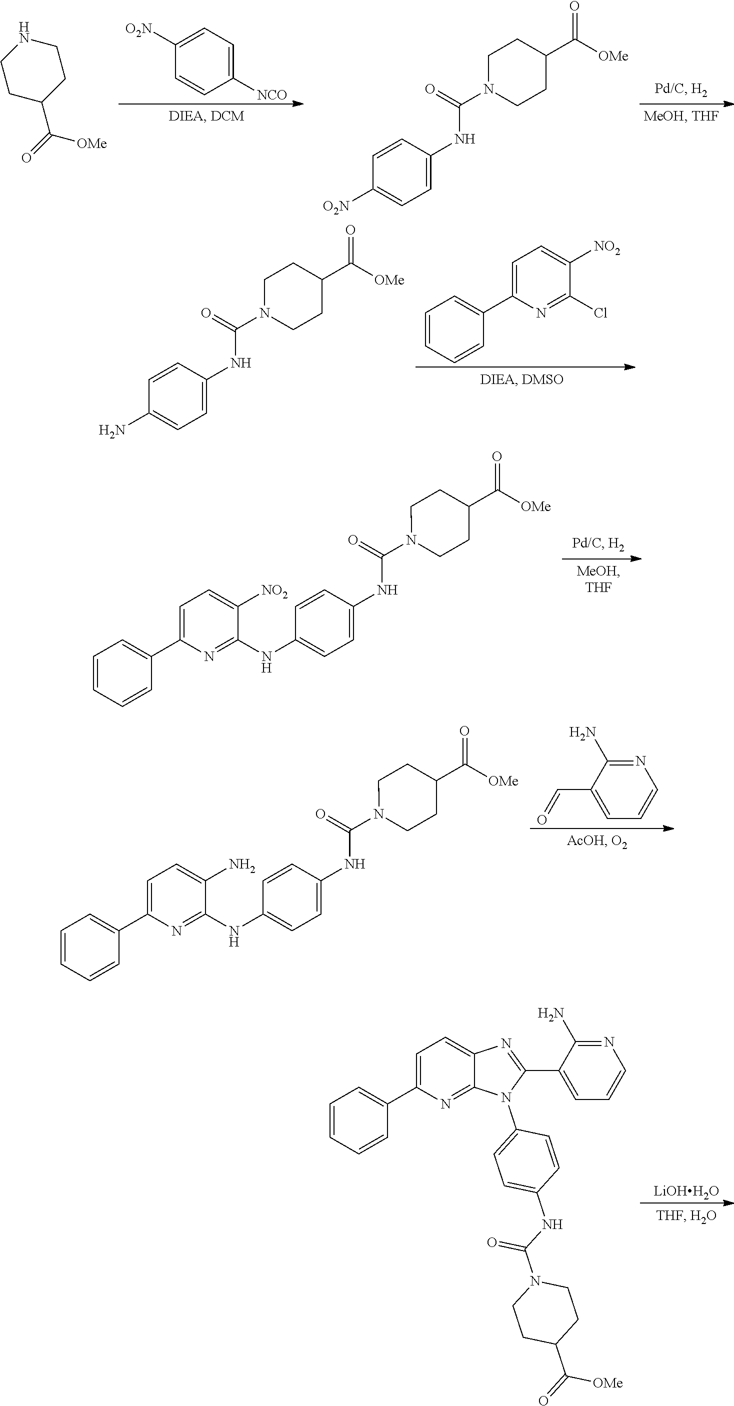

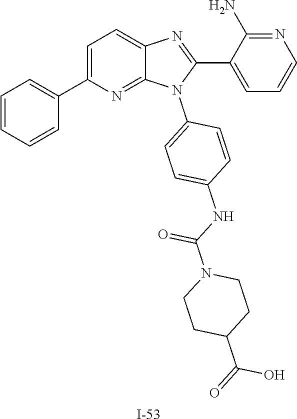

Step 1. To a stirred solution of methyl piperidine-4-carboxylate (2.00 g, 14.0 mmol) and DIEA (5.1 mL, 27.9 mmol) in DCM (30 mL), 1-isocyanato-4-nitro-benzene (2.29 g, 14.0 mmol) was added dropwise at 0° C. and the reaction mixture was subsequently stirred at 25° C. under N₂ for 16 hrs. The reaction mixture was concentrated and purified by flash chromatography on silica gel (10% methanol in DCM) to afford methyl 1-[(4-nitrophenyl)carbamoyl]piperidine-4-carboxylate (2.00 g, 46.6% yield) as a yellow solid. ¹H NMR (400 MHz, DMSO-d₆) δ=9.21 (s, 1H), 8.20-8.07 (m, 2H), 7.77-7.64 (m, 2H), 4.09-4.02 (m, 2H), 3.62 (s, 3H), 3.04-2.88 (m, 2H), 2.72-2.56 (m, 1H), 1.92-1.80 (m, 2H), 1.60-1.43 (m, 2H).

Step 2. To a stirred solution of methyl 1-[(4-nitrophenyl)carbamoyl]piperidine-4-carboxylate (2.00 g, 6.51 mmol) in methanol (10 mL) and THF (10 mL), Pd/C (200 mg, 50% wet by water, 10% pd) was added under argon. The reaction was degassed and purged with argon (×3), then degassed and purged with H₂ (×3) and stirred under H_{z}(50 psi) for 10 hrs at 50° C. The reaction mixture was filtered and the filtrate was concentrated to afford methyl 1-[(4-aminophenyl)carbamoyl]piperidine-4-carboxylate (2.0 g, crude) as a yellow solid. ¹H NMR (400 MHz, DMSO-d₆) δ=8.05 (s, 1H), 7.09-6.88 (m, 2H), 6.55-6.33 (m, 2H), 4.70 (s, 2H), 4.04-3.86 (m, 2H), 3.61 (s, 3H), 2.90-2.73 (m, 2H), 2.59-2.52 (m, 1H), 1.90-1.74 (m, 2H), 1.54-1.39 (m, 2H)

Step 3. To a stirred solution of 2-chloro-3-nitro-6-phenyl-pyridine (846 mg, 3.61 mmol) and methyl 1-[(4-aminophenyl)carbamoyl]piperidine-4-carboxylate (1.00 g, 3.61 mmol) in DMSO (10 mL), DIEA (3.3 mL, 18.0 mmol) was added and the reaction mixture was stirred at 100° C. under N₂ for 10 hrs. The reaction mixture was diluted with EtOAc (50 mL), washed with water (50 mL) and brine (50 mL×2), dried over anhydrous sodium sulfate, filtered and the filtrate was concentrated to afford a residue. The crude product was purified by flash chromatography on silica gel (60-80% of EtOAc in PE) to afford methyl 1-[[4-[(3-nitro-6-phenyl-2-pyridyl)amino]phenyl]carbamoyl]piperidine-4-carboxylate (500 mg, 29.2% yield) as a yellow oil. ¹H NMR (400 MHz, DMSO-d₆) δ=10.05 (s, 1H), 8.65-8.51 (m, 2H), 8.09 (dd, J=2.8, 6.8 Hz, 2H), 7.65-7.43 (m, 8H), 4.09-4.03 (m, 2H), 3.63 (s, 3H), 3.00-2.86 (m, 2H), 2.70-2.55 (m, 1H), 1.93-1.78 (m, 2H), 1.60-1.40 (m, 2H).

Step 4. To a stirred solution of methyl 1-[[4-[(3-nitro-6-phenyl-2-pyridyl)amino]phenyl]carbamoyl]piperidine-4-carboxylate (500 mg, 1.05 mmol) in methanol (5 mL) and THF (5 mL), Pd/C (100 mg, wet, 10%) was added under argon. The reaction was degassed and purged with argon (×3), then degassed and purged with H₂ (×3) and stirred under H₂ (50 psi) for 10 hrs at 50° C. Upon completion the mixture was filtered under argon to afford methyl 1-[[4-[(3-amino-6-phenyl-2-pyridyl)amino]phenyl]carbamoyl]piperidine-4-carboxylate (300 mg, 64.0% yield) as a yellow solid. ¹H NMR (400 MHz, DMSO-d₆) δ=8.35 (s, 1H), 7.98-7.83 (m, 2H), 7.72 (s, 1H), 7.63 (d, J=9.2 Hz, 2H), 7.43-7.31 (m, 4H), 7.28-7.17 (m, 2H), 6.94 (d, J=8.0 Hz, 1H), 5.22 (s, 2H), 4.03 (d, J=13.6 Hz, 2H), 3.63 (s, 3H), 2.90 (s, 2H), 2.63-2.54 (m, 1H), 1.88-1.80 (m, 2H), 1.60-1.40 (m, 2H).

Step 5. To a stirred solution of methyl 1-[[4-[(3-amino-6-phenyl-2-pyridyl)amino]phenyl]carbamoyl]piperidine-4-carboxylate (250 mg, 0.56 mmol) in acetic acid (2 mL), 2-aminopyridine-3-carbaldehyde (18 mg, 0.15 mmol) was added and the reaction mixture was stirred at 80° C. under O₂ for 1 hr. The reaction mixture was diluted with EtOAc (10 mL), washed with water (10 mL) and brine (20 mL×2), dried over anhydrous sodium sulfate, filtered and the filtrate was concentrated to afford a residue. The crude product was purified by prep-HPLC (column. Welch Xtimate C18 150×30 mm×5 mm, method: water (FA)-ACN, begin B: 25, end B 65) to afford methyl 1-[[4-[2-(2-amino-3-pyridyl)-5-phenyl-imidazo[4,5-b]pyridin-3-yl]phenyl]carbamoyl]piperidine-4-carboxylate (30.0 mg, 0.055 mmol) as a white solid. ¹H NMR (400 MHz, DMSO-d₆) δ=8.76 (s, 1H), 8.25 (d, J=8.4 Hz, 1H), 8.08-7.93 (m, 4H), 7.63 (d, J=8.8 Hz, 2H), 7.52-7.30 (m, 6H), 7.25 (dd, J=2.0, 7.6 Hz, 1H), 7.07 (s, 2H), 6.43 (dd, J=4.8, 7.6 Hz, 1H), 4.16-3.95 (m, 2H), 3.63 (s, 3H), 3.04-2.89 (m, 2H), 2.65-2.55 (m, 1H), 1.95-1.80 (m, 2H), 1.61-1.44 (m, 2H).

Step 6. To a stirred solution of methyl 1-[[4-[2-(2-amino-3-pyridyl)-5-phenyl-imidazo[4,5-b]pyridin-3-yl]phenyl]carbamoyl]piperidine-4-carboxylate (30.0 mg, 0.055 mmol) in THF (0.5 mL) and water (0.5 mL), LiOH·H₂O (4.4 mg, 0.11 mmol) was added and the reaction mixture was stirred at 25° C. under N₂ for 2 hrs. The reaction mixture was concentrated and the pH adjusted to pH ~-5 with 1N HCl to afford a residue. The crude product was purified by prep-HPLC (column: Welch Xtimate C18 150×30 mm×5 mm, method: water (FA)-ACN, begin B: 25, end B: 56) to afford 1-[[4-[2-(2-amino-3-pyridyl)-5-phenyl-imidazo[4,5-b]pyridin-3-yl]phenyl]carbamoyl]piperidine-4-carboxylic acid 1-53 (7.10 mg, 24.3% yield) as a white solid. ¹H NMR (400 MHz, DMSO-d₆) δ=12.30 (s, 1H), 8.76 (s, 1H), 8.25 (d, J=8.4 Hz, 1H), 8.08-7.91 (m, 4H), 7.64 (d, J=8.8 Hz, 2H), 7.54-7.20 (m, 6H), 7.08 (s, 2H), 6.43 (dd, J=4.8, 7.6 Hz, 1H), 4.05 (d, J=13.2 Hz, 2H), 2.95 (t, J=11.2 Hz, 2H), 2.49-2.42 (m, 1H), 1.86 (d, J=10.4 Hz, 2H), 1.61-1.35 (m, 2H). HPLC Rₜ=2.839 min in 8 min chromatography, purity 99.1%. LCMS Rₜ=2.382 min in 4 min chromatography, purity 99.070%, MS ESI calcd. for 533.22 [M+H]⁺ 534.22, found 534.2.

### Example 48. Synthesis of (1r,4r)-4-((3-(4-(2-(2-aminopyridin-3-yl)-5-phenyl-3H-imidazo[4,5-b]pyridin-3-yl)phenyl)azetidin-1-yl)methyl)cyclohexane-1-carboxylic acid (Compound I-57)

### Synthesis of trans-methyl 4-formylcyclohexanecarboxylate

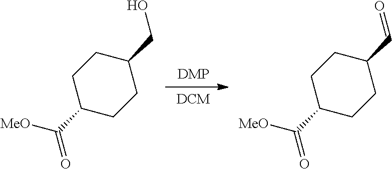 [SMILES: COC(=O)[C@H]1CC[C@H](CO)CC1 ; COC(=O)[C@H]1CC[C@H](C=O)CC1]

Step 1. To a solution of trans-methyl 4-(hydroxymethyl) cyclohexanecarboxylate (200 mg, 1.16 mmol) in DCM (5.00 mL), DMP (0.990 g, 2.32 mmol) was added at 0° C. and the mixture stirred at 25° C. for 2 hrs. The mixture was quenched with 1 M Na₂S₂O₃:NaHCO₃ (1/1, 20 mL), and stirred for an additional 30 mins. The mixture was extracted with DCM (20 mL×3) and the organic phase was washed with brine (20 mL), dried with Na₂SO₄, filtered, and concentrated to give trans-methyl 4-formylcyclohexanecarboxylate (190 mg, 96.1% yield) as a white solid. ¹H NMR (DMSO-d₆, 400 MHz) δ_{H}=9.55 (d, J=0.8 Hz, 1H), 3.59 (s, 3H), 1.95 (br d, J=2.0 Hz, 2H), 1.92 (br d, J=1.2 Hz, 2H), 1.44-1.31 (m, 4H), 1.27-1.15 (m, 2H).

### Synthesis of (1r,4r)-4-((3-(4-(2-(2-aminopyridin-3-yl)-S-phenyl-3H-imidazo[4,5-b]pyridin-3-yl)phenyl)azetidin-1-yl)methyl)cyclohexane-1-carboxylic acid (Compound I-57)

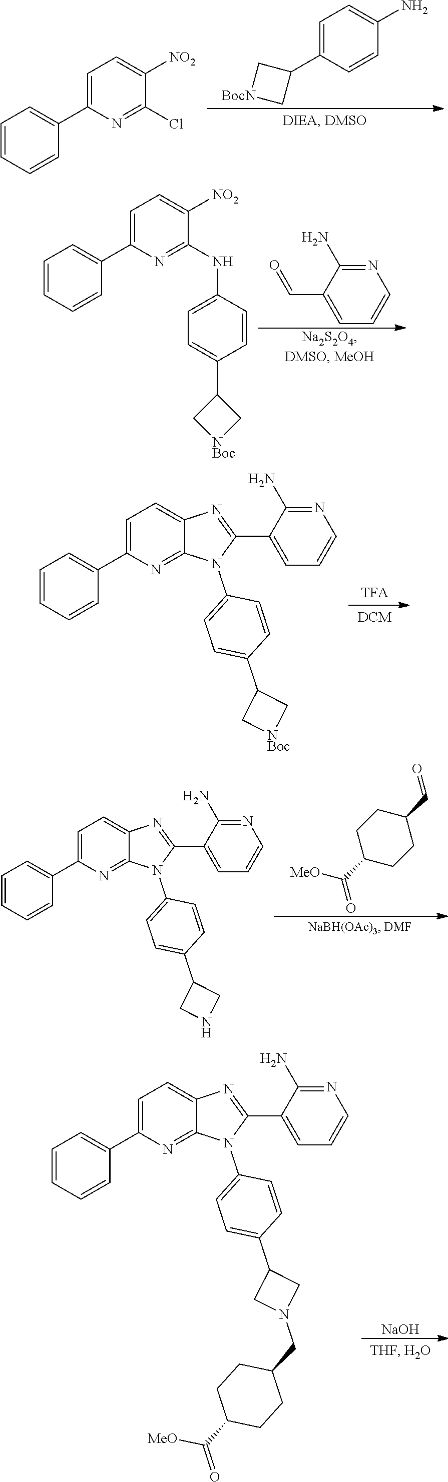

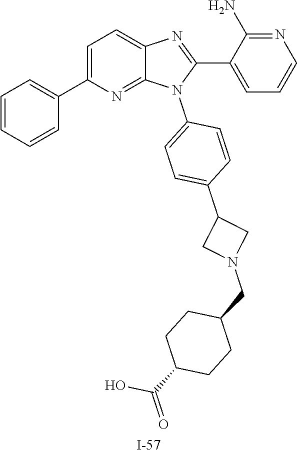

Step 1. To a solution of 2-chloro-3-nitro-6-phenylpyridine (2.00 g, 8.05 mmol) in DMSO (20.0 mL), tert-butyl 3-(4-aminophenyl)azetidine-1-carboxylate (2.00 g, 8.05 mmol) and DIEA (2.10 mL, 12.3 mmol) were added at 25° C. and the mixture was stirred at 80° C. for 16 hrs. Water (200 mL) was added to the residue and the resulting mixture was extracted with EtOAc (200 mL×3). The combined organic phase was washed with brine (50 mL), water (50 mL), dried over anhydrous Na₂SO₄, filtered and concentrated. The residue was purified by flash silica gel chromatography (eluent of 0-33% EtOAc/PE @45 mL/min) to give tert-butyl 3-(4-((3-nitro-6-phenylpyridin-2-yl)amino)phenyl)azetidine-1-carboxylate (1.20 g, 67.3% yield) as a red solid. ¹H NMR (400 MHz, CDCl₃) δ=10.31 (s, 1H), 8.59 (d, J=8.8 Hz, 1H), 8.10-8.00 (m, 2H), 7.76 (d, J=8.4 Hz, 2H), 7.57-7.45 (m, 3H), 7.38 (d, J=8.4 Hz, 2H), 7.31 (d, J=8.8 Hz, 1H), 4.36 (t, J=8.8 Hz, 2H), 4.02 (dd, J=8.4, 6.0 Hz, 2H), 3.82-3.73 (m, 1H), 1.49 (s, 9H).

Step 2. To a solution of tert-butyl 3-(4-((3-nitro-6-phenylpyridin-2-yl)amino)phenyl)azetidine-1-carboxylate (2.30 g, 5.15 mmol) in DMSO (23.0 mL) and methanol (23.0 mL), 2-aminonicotinaldehyde (849 mg, 6.95 mmol) and Na₂S₂O₄ (1.79 g, 10.3 mmol) were added at 25° C., and the mixture was stirred at 100° C. for 16 hrs. The reaction mixture was concentrated to remove MeOH, water (50 mL) was added to the residue, and the resulting mixture was extracted with EtOAc (100 ml, 3). The combined organic phase was washed with brine (20 mL), water (20 mL), dried over anhydrous Na₂SO₄, filtered and concentrated. The residue was purified by flash silica gel chromatography (eluent of 0-66% EtOAc in PE) to give tert-butyl 3-(4-(2-(2-aminopyridin-3-yl)-5-phenyl-3H-imidazo[4,5-b]pyridin-3-yl)phenyl)azetidine-1-carboxylate (400 mg, 17.1% yield) as a yellow oil. ¹H NMR (400 MHz, DMSO-d₆) δ=8.74 (d, J=2.4 Hz, 1H), 8.71 (d, J=2.4 Hz, 1H), 8.34-8.28 (m, 3H), 8.08 (dd, J=8.4, 2.0 Hz, 2H), 7.94 (dd, J=4.8, 2.0 Hz, 1H), 7.51 (d, J=8.4 Hz, 2H), 7.39 (dd, J=8.4, 6.4 Hz, 4H), 7.13 (dd, J=7.6, 1.6 Hz, 1H), 6.49-6.43 (m, 1H), 3.17 (s, 2H), 3.16 (s, 2H), 3.05 (s, 1H), 1.40 (s, 9H).

Step 3. To a solution of tert-butyl 3-(4-(2-(2-aminopyridin-3-yl)-5-phenyl-3H-imidazo[4,5-b]pyridin-3-yl)phenyl)azetidine-1-carboxylate (200 mg, 0.386 mmol) in DCM (4.00 mL), TFA (2.00 mL) was added at 25° C. and the mixture was stirred for 2 hrs. The reaction mixture was concentrated to give 3-(3-(4-(azetidin-3-yl)phenyl)-5-phenyl-3H-imidazo[4,5-b]pyridin-2-yl)pyridin-2-amine (161 mg, 99.8% yield, TFA salt) as a brown oil, which was used in the next step directly. LCMS Rₜ=0.410 min in 1 min chromatography, purity 79.5%, MS ESI calcd. For 418.19 [M+H]⁺ 419.19, found 419.1.

Step 4. To a solution of 3-[3-[4-(azetidin-3-yl)phenyl]-5-phenyl-imidazo[4,5-b]pyridin-2-yl]pyridin-2-amine (80.0 mg, 0.191 mmol) in DMF (10.0 mL), TEA was added and the solution adjusted to pH ~7. To the mixture, trans-methyl 4-formylcyclohexanecarboxylate (163 mg, 0.956 mmol) was added and the mixture (pH ~7) was stirred at 25° C. for 16 hrs. NaBH(OAc)₃ (203 mg, 0.956 mmol) was added and the mixture was stirred at 25° C. for an additional 16 hrs. To the mixture, water (30 mL) was added and the whole was extracted with EtOAc (30 mL×3). The organic phase was washed with brine (10 mL×2), dried with Na₂SO₄, filtered, and concentrated. The mixture was purified by column (DCM/MeOH=0-20%) to give trans-methyl 4-[[3-[4-[2-(2-amino-3-pyridyl)-5-phenyl-imidazo[4,5-b]pyridin-3-yl]phenyl]azetidin-1-yl]methyl]cyclohexanecarboxylate (50.0 mg, 40.2% yield) as a yellow solid. LCMS Rₜ=0.46 min in 1 min chromatography, Xtimate C18 2.1*30 mm, 3 μm, purity 88.1%, MS ESI calcd. for 572.29 [M+H]⁺ 573.29, found 573.2.

Step 5. To a solution of trans-methyl 4-[[3-[4-[2-(2-amino-3-pyridyl)-5-phenyl-imidazo[4,5-b]pyridin-3-yl]phenyl]azetidin-1-yl]methyl]cyclohexanecarboxylate (25.0 mg, 0.0437 mmol) in THF (1.00 mL) and water (1.00 ml), NaOH (3.50 mg, 0.0873 mmol) was added and the mixture was stirred at 25° C. for 16 hrs. The mixture was concentrated to remove THF, and the residue was dissolved with DMSO and adjusted to pH ~~5. The mixture was purified by prep-HPLC (Xtimate C18 150*40 mm*5 μm, water (HCl)-ACN) to give (1r,4r)-4-((3-(4-(2-(2-aminopyridin-3-yl)-5-phenyl-3H-imidazo[4,5-b]pyridin-3-yl)phenyl)azetidin-1-yl)methyl)cyclohexane-1-carboxylic acid 1-57 (8.10 mg, 31.4% yield) as a yellow solid. ¹H NMR (DMSO-d₆, 400 MHz) δ_{H}=8.32 (d, J=8.4 Hz, 1H), 8.08-7.96 (m, 4H), 7.72 (br d, J=6.4 Hz, 1H), 7.66-7.53 (m, 4H), 7.51-7.37 (m, 3H), 6.77 (br t, J=6.8 Hz, 1H), 4.61-4.07 (m, 5H), 3.20 (br d, J=6.4 Hz, 1H), 3.12 (br d, J=6.4 Hz, 1H), 2.14 (br t, J=12.0 Hz, 1H), 1.90 (br d, J=11.2 Hz, 2H), 1.83-1.70 (m, 2H), 1.67-1.47 (m, 1H), 1.39-1.19 (m, 2H), 1.10-0.87 (m, 2H). HPLC Rₜ=3.556 min in 8 min chromatography, 0-60AB, purity 94.4%. LCMS Rₜ=2.609 min in 4 min chromatography, Xtimate C18 2.1*30 mm, 3 μm, purity 95.4%, MS ESI calcd for 558.27 [M+H]⁺ 559.27, found 559.1.

### Example 49 and 51. Synthesis of (1S,3R)-3-((3-(4-(2-(2-aminopyridin-3-yl)-5-phenyl-3H-imidazo[4,5-b]pyridin-3-yl)phenyl)azetidin-1-yl)methyl)cyclopentane-1-carboxylic acid (Compound I-58) and (1R,3R)-3-((3-(4-(2-(2-aminopyridin-3-yl)-5-phenyl-3H-imidazo[4,5-b]pyridin-3-yl)phenyl)azetidin-1-yl)methyl)cyclopentane-1-carboxylic acid (Compound I-61)

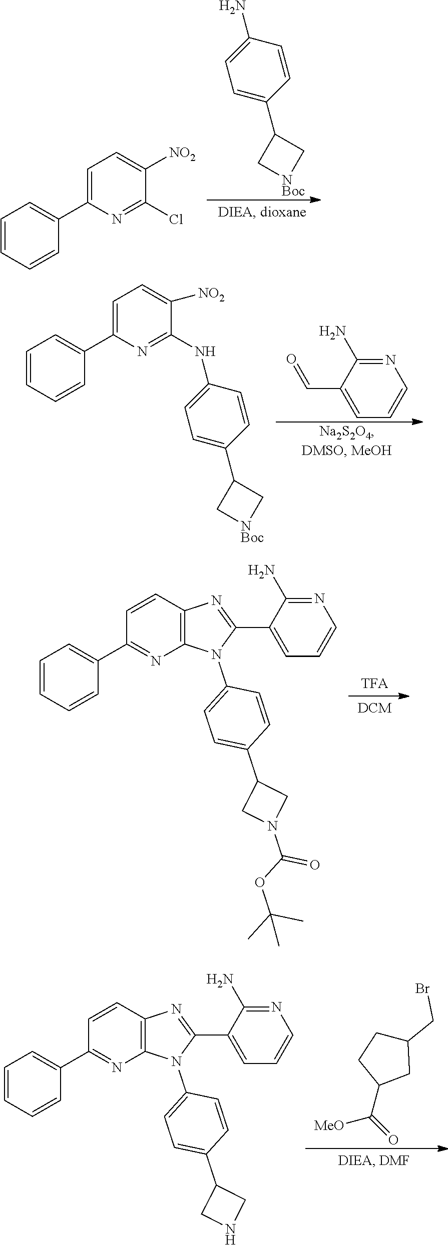

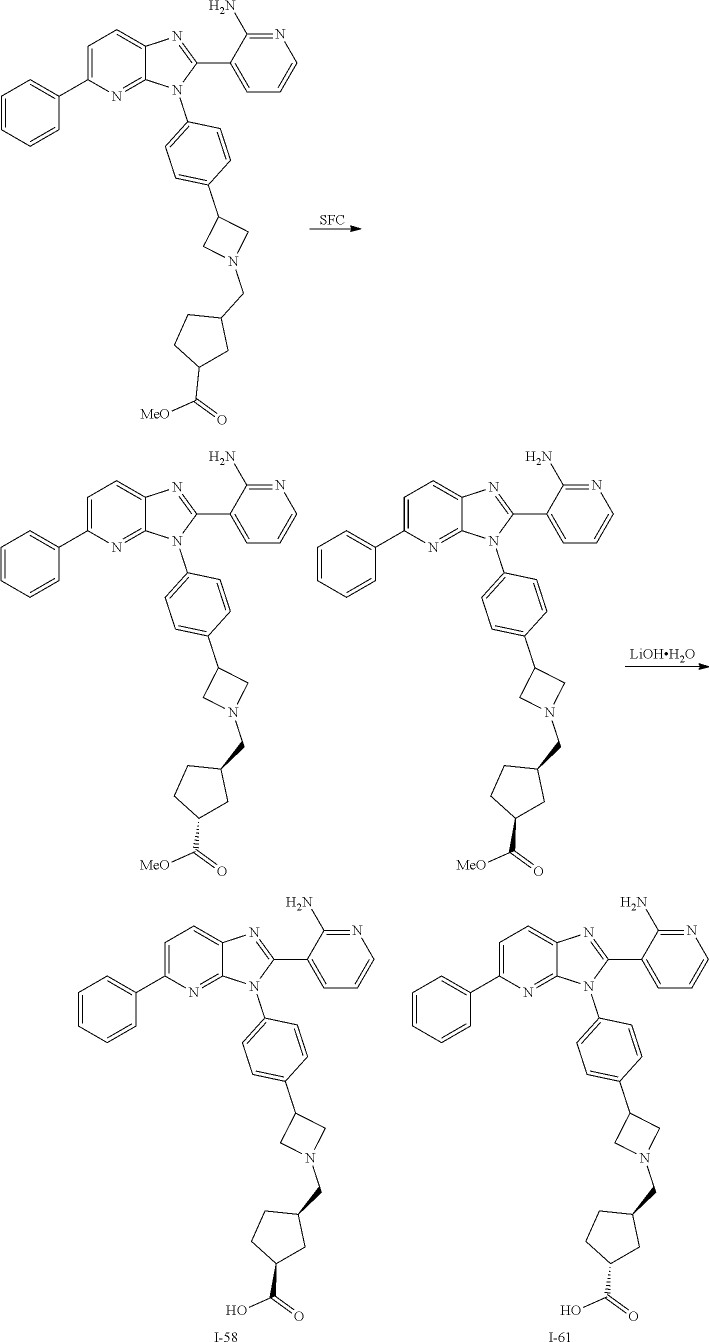

Step 1. To a solution of 2-chloro-3-nitro-6-phenylpyridine (4.00 g, 17.0 mmol) and tert-butyl 3-(4-aminophenyl)azetidine-1-carboxylate (5.08 g, 20.5 mmol) in 1,4-dioxane (40.0 mL), DIEA (8.50 mL, 51.1 mmol) was added and the mixture was stirred at 80° C. for 12 hrs. Water (20 mL) was added to the reaction mixture, and the whole was extracted with EtOAc (100 mL×3). The combined organic phase was washed with brine (100 mL), dried over anhydrous Na₂SO₄, filtered and concentrated. The residue was purified by flash silica gel chromatography (eluent of 0-15% EtOAc in PE) to afford tert-butyl 3-(4-((3-nitro-6-phenylpyridin-2-yl)amino)phenyl)azetidine-1-carboxylate (2.70 g, 33.6% yield) as a yellow oil. ¹H NMR (DMSO-d₆, 400 MHz) δ_{H}=10.12 (s, 1H), 8.61 (d, J=8.8 Hz, 1H), 8.14-8.07 (m, 2H), 7.76 (d, J=8.4 Hz, 2H), 7.61-7.51 (m, 4H), 7.40 (d, J=8.4 Hz, 2H), 4.33-4.21 (m, 2H), 3.91-3.77 (m, 3H), 1.41 (s, 9H).

Step 2. To a solution of tert-butyl 3-(4-((3-nitro-6-phenylpyridin-2-yl)amino)phenyl)azetidine-1-carboxylate (2.00 g, 4.48 mmol) and 2-aminonicotinaldehyde (0.74 g, 6.05 mmol) in DMSO (10.0 mL) and methanol (10.0 mL), Na₂S₂O₄ (2.34 g, 13.4 mmol) was added under N₂ and the reaction mixture was stirred at 100° C. for 12 hrs. The reaction mixture was concentrated and aq. LiCl (100 mL) was added to the residue and the resulting mixture was extracted with EtOAc (100 mL 3). The combined organic phase was washed with brine (100 mL), dried over anhydrous Na₂SO₄, filtered and concentrated. The residue was purified by prep-HPLC (Column: Welch Xtimate C18 150*30 mm*5 μm; mobile phase: [water (NH₃H₂O+NH₄HCO₃)-ACN]; B %: 26%-66%; 20 min) to afford tert-butyl 3-(4-(2-(2-aminopyridin-3-yl)-5-phenyl-3H-imidazo[4,5-b]pyridin-3-yl)phenyl)azetidine-1-carboxylate (11.4 mg, 2.60% yield) as a white solid. ¹H NMR (400 MHz, DMSO-d₆) δ=8.27 (d, J=8.4 Hz, 1H), 8.06-7.96 (m, 4H), 7.57-7.35 (m, 7H), 7.20 (dd, J=7.6, 1.6 Hz, 1H), 6.95 (s, 2H), 6.43 (dd, J=7.6, 4.8 Hz, 1H), 4.35-4.24 (m, 2H), 3.95-3.87 (m, 3H), 1.41 (s, 9H). HPLC Rₜ=3.561 min in 8 min chromatography, purity 100% LCMS Rₜ=1.599 min in 2.5 min chromatography, purity 100.00%, MS ESI calcd. for 518.24, [M+H]⁺ 519.24, found 519.3.

Step 3. To a solution of tert-butyl 3-(4-(2-(2-aminopyridin-3-yl)-5-phenyl-3H-imidazo[4,5-b]pyridin-3-yl)phenyl)azetidine-1-carboxylate (500 mg, 0.964 mmol) in DCM (5.00 mL), TFA (0.500 mL, 0.382 mmol) was added and the mixture was stirred at 25° C. for 2 hrs. The mixture was concentrated directly to give 3-(3-(4-(azetidin-3-yl)phenyl)-5-phenyl-3H-imidazo[4,5-b]pyridin-2-yl)pyridin-2-amine (300 mg, 0.645 mmol) as a yellow oil, which was used directly in the next step. LCMS Rₜ=0.499 min in 1 min chromatography, purity 90.2%, MS ESI calcd. for 418.50, [M+H]⁺ 419.50, found 419.1.

Step 4. To a solution of 3-[3-[4-(azetidin-3-yl) phenyl]-5-phenyl-imidazo[4,5-b]pyridin-2-yl]pyridin-2-amine (500 mg, 1.19 mmol) and methyl 3-(bromomethyl) cyclopentanecarboxylate (396 mg, 1.79 mmol) in DMF (5.00 mL), DIEA (0.590 mL, 3.58 mmol) was added and the mixture was stirred at 60° C. for 16 hrs. Water (20 mL) was then added to the mixture and the whole was extracted with EtOAc (20 mL 3). The organic phase was washed with brine (20 mL), dried with Na₂SO₄, and concentrated to give methyl 3-[[3-[4-[2-(2-amino-3-pyridyl)-5-phenyl-imidazo[4,5-b]pyridin-3-yl]phenyl]azetidin-1-yl]methyl]cyclopentanecarboxylate (400 mg, 59.9% yield) as a yellow solid. LCMS Rₜ=0.611 min and 0.622 min in 1 min chromatography, Xtimate C18 2.1*30 mm,3 μm, purity 99.2%, MS ESI calcd. for 558.27 [M+H]⁺ 559.27, found 559.2.

Step 5. Methyl 3-[[3-[4-[2-(2-amino-3-pyridyl)-5-phenyl-imidazo[4,5-b]pyridin-3-yl]phenyl]azetidin-1-yl]methyl]cyclopentanecarboxylate (400 mg, 0.716 mmol) was purified by SFC (DAICEL CHIRAL PAK AD (250 mm*30 mm,10 μm), CO₂-i-PrOH(0.1% NH₃H₂O)) to give trans-methyl (1R,3R)-3-[[3-[4-[2-(2-amino-3-pyridyl)-5-phenyl-imidazo[4,5-b]pyridin-3-yl]phenyl]azetidin-1-yl]methyl]cyclopentanecarboxylate (30.0 mg, 7.50% yield) as a yellow solid and cis-methyl (1S,3R)-3-[[3-[4-[2-(2-amino-3-pyridyl)-5-phenyl-imidazo[4,5-b]pyridin-3-yl]phenyl]azetidin-1-yl]methyl]cyclopentanecarboxylate (30.0 mg, 7.50% yield) as a yellow solid. LCMS Rₜ=2.290 min in 4 min chromatography, Xtimate C18 2.1*30 mm,3 um, purity 100%, MS ESI calcd. for 558.27 [M+Na]⁺ 581.27, found 581.2. LCMS Rₜ=2.278 min in 4 min chromatography, Xtimate C18 2.1*30 mm,3 um, purity 100%, MS ESI calcd. for 558.27 [M+Na]⁺ 581.27, found 581.1.

Step 6a. To a solution of trans-methyl (1R,3R)-3-[[3-[4-[2-(2-amino-3-pyridyl)-5-phenyl-imidazo[4,5-b]pyridin-3-yl]phenyl]azetidin-1-yl]methyl]cyclopentanecarboxylate (30.0 mg, 0.0537 mmol) in THF (1.00 mL) and water (1.00 mL), LiOH·H₂O (6.80 mg, 0.161 mmol) was added and the mixture was stirred at 25° C. for 12 hrs. The mixture was concentrated to remove THF. The mixture was then dissolved with DMSO and 1 M HCl, and then purified by prep-HPLC (Welch Xtimate C18 150*30 mm*5 μm, water (FA)-ACN) to give (1R,3R)-3-((3-(4-(2-(2-aminopyridin-3-yl)-5-phenyl-3H-imidazo[4,5-b]pyridin-3-yl)phenyl)azetidin-1-yl)methyl)cyclopentane-1-carboxylic acid I-61 (20.7 mg, 70.8% yield) as a yellow solid. ¹H NMR (DMSO-d₆ 400 MHz) δ_{H}=8.32-8.19 (m, 1H), 8.08-7.92 (m, 4H), 7.59-7.33 (m, 7H), 7.22 (dd, J=1.6, 7.6 Hz, 1H), 6.44 (dd, J=4.8, 7.6 Hz, 1H), 3.84-3.73 (m, 3H), 3.36-3.24 (m, 2H), 2.71-2.64 (m, 1H), 2.57 (br d, J=6.4 Hz, 2H), 2.07-1.88 (m, 2H), 1.82-1.65 (m, 3H), 1.41-1.19 (m, 2H). HPLC Rₜ=3.593 in 8 min chromatography, 0-60AB, purity 100%. LCMS Rₜ=2.788 min in 4 min chromatography, Xtimate C18 2.1*30 mm,3 um, purity 100%, MS ESI calcd. for 544.26 [M+H]⁺ 545.26, found 545.3.

Step 6b. To a solution of cis-methyl (1S,3R)-3-[[3-[4-[2-(2-amino-3-pyridyl)-5-phenyl-imidazo[4,5-b]pyridin-3-yl]phenyl]azetidin-1-yl]methyl]cyclopentanecarboxylate (30.0 mg, 0.0537 mmol) in THF (0.500 mL) and water (0.500 mL), LiOH·H₂O (6.80 mg, 0.161 mmol) was added and the mixture was stirred at 25° C. for 16 hrs. The mixture was concentrated to remove THF. The residue was then dissolved with DMSO and 1 M HCl, and subsequently purified by prep-HPLC (Xtimate C18 150*40 mm*5 μm, water(FA)-ACN) to give (1S,3R)-3-((3-(4-(2-(2-aminopyridin-3-yl)-5-phenyl-3H-imidazo[4,5-b]pyridin-3-yl)phenyl)azetidin-1-yl)methyl)cyclopentane-1-carboxylic acid 1-58 (24.7 mg, 84.5% yield) as a yellow solid. ¹H NMR (DMSO-d₆, 400 MHz) δ_{H}=8.26 (d, J=8.4 Hz, 1H), 8.03-7.92 (m, 4H), 7.63-7.56 (m, 2H), 7.55-7.49 (m, 2H), 7.48-7.43 (m, 2H), 7.42-7.37 (m, 1H), 7.30 (dd, J=16, 7.6 Hz, 1H), 6.48 (dd, J=5.2, 7.6 Hz, 1H), 4.50-4.45 (m, 1H), 4.38 (br dd, J=2.0, 3.6 Hz, 1H), 4.30-4.06 (m, 3H), 3.38-3.19 (m, 2H), 2.82-2.67 (m, 1H), 2.20-1.96 (m, 2H), 1.91-1.70 (m, 3H), 1.46-1.23 (m, 2H). HPLC Rₜ=3.611 in 8 min chromatography, 0-60AB, purity 100%. LCMS Rₜ=2.809 min in 4 min chromatography, Xtimate C18 2.1*30 mm,3 μm, purity 100%, MS ESI calcd. for 544.26 [M+H]⁺ 545.26, found 545.2.

### Example 51 and 52. Synthesis of (1R,4r-((R)-1-(3-(4-(2-(2-aminopyridin-3-yl)-5-phenyl-3H-imidazo[4,5-b]pyridin-3-yl)phenyl)azetidin-1-yl)ethyl)cyclohexane-1-carboxylic acid (Compound I-59) and (1S,4r)-4-((S)-1-(3-(4-(2-(2-aminopyridin-3-yl)-5-phenyl-3H-imidazo[4,5-b]pyridin-3-yl)phenyl)azetidin-1-yl)ethyl)cyclohexane-1-carboxylic acid (Compound I-60)

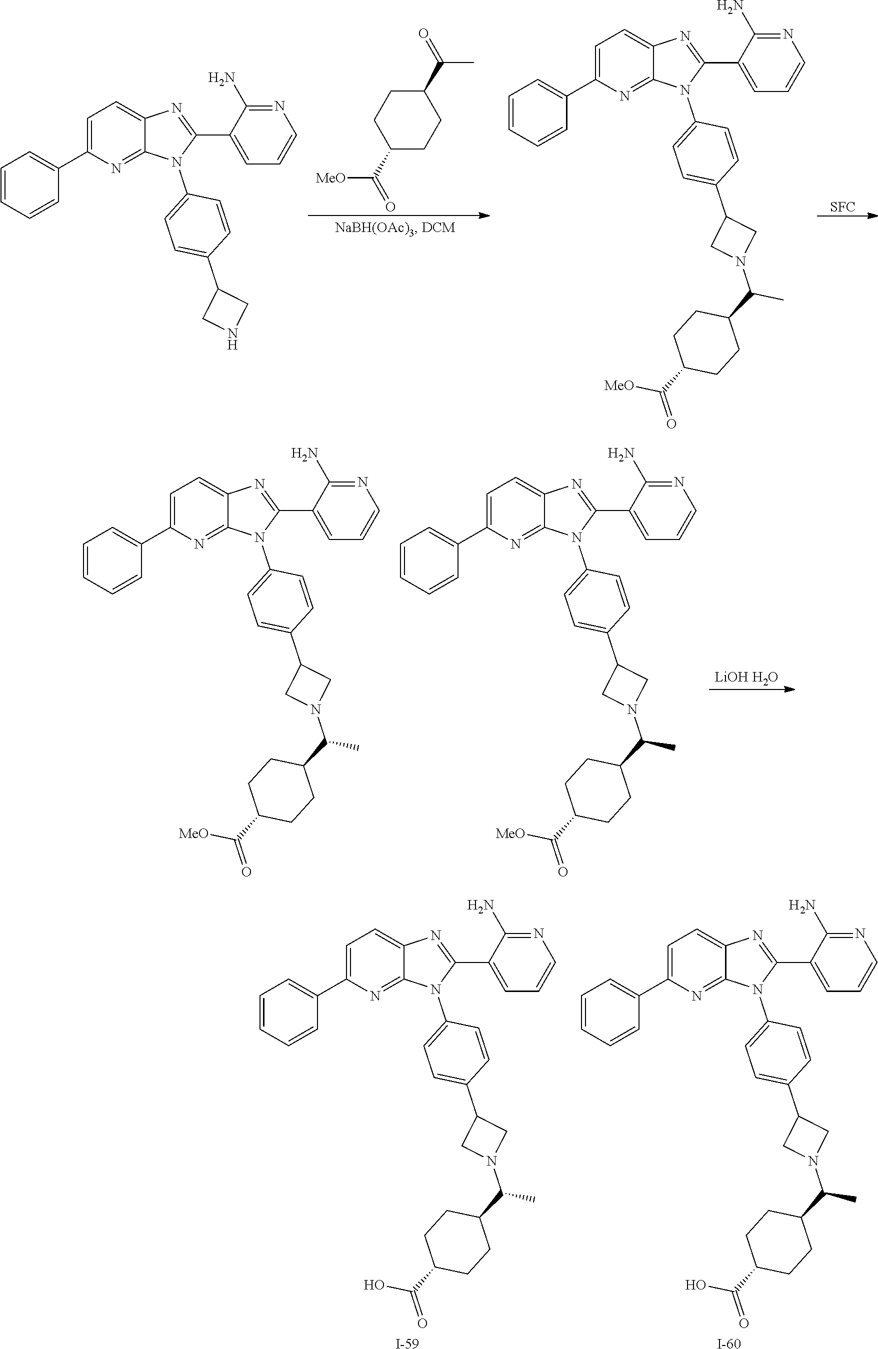

Step 1. A mixture of 3-[3-[4-(azetidin-3-yl) phenyl]-5-phenyl-imidazo[4,5-b]pyridin-2-yl]pyridin-2-amine (500 mg, 1.19 mmol) and trans-methyl 4-acetylcyclohexanecarboxylate (264 mg, 1.43 mmol) in DCM (10.0 mL) was stirred at 25° C. for 30 mins. To the mixture NaBH(OAc)₃ (756 mg, 3.58 mmol) was added and the mixture was stirred at 25° C. for 16 hrs. Water (10 mL) was added to the mixture, and the whole was extracted with DCM (20 mL×3). The organic phase was washed with brine (10 mL), dried with Na₂SO₄, and concentrated. The residue was purified by column (DCM:MeOH=0-10%) to give trans-methyl 4-[1-[3-[4-[2-(2-amino-3-pyridyl)-5-phenyl-imidazo[4,5-b]pyridin-3-yl]phenyl]azetidin-1-yl]ethyl]cyclohexanecarboxylate (330 mg, 47.1% yield) as a yellow solid. LCMS Rₜ=4.549 min in 7 min chromatography, Xtimate C18 2.1*30 mm,3 μm, purity 99.1%, MS ESI calcd. for 586.31 [M+H]⁺ 587.31, found 587.3.

Step 2. Trans-methyl 4-[1-[3-[4-[2-(2-amino-3-pyridyl)-5-phenyl-imidazo[4,5-b]pyridin-3-yl]phenyl]azetidin-1-yl]ethyl]cyclohexanecarboxylate (300 mg, 0.511 mmol) was purified by SFC (DAICEL CHIRALPAK AD (250 mm*30 mm, 10 μm), CO₂-i-PrOH (0.1% NH₃H₂O) to give trans-methyl 4-[(1R)-1-[3-[4-[2-(2-amino-3-pyridyl)-5-phenyl-imidazo[4,5-b]pyridin-3-yl]phenyl]azetidin-1-yl]ethyl]cyclohexanecarboxylate (80 mg, 26.7% yield) as a yellow solid and trans-methyl 4-[(1S)-1-[3-[4-[2-(2-amino-3-pyridyl)-5-phenyl-imidazo[4,5-b]pyridin-3-yl]phenyl]azetidin-1-yl]ethyl]cyclohexanecarboxylate (100 mg, 33.3% yield) as a yellow solid. LCMS Rₜ=0.635 min in 1 min chromatography, Xtimate C18 2.1*30 mm, 3 μm, purity 100%, MS ESI calcd. for 586.31 [M+H]⁺ 587.31, found 587.3. LCMS Rₜ=0.642 min in 1 min chromatography, Xtimate C18 2.1*30 mm,3 μm, purity 100%, MS ESI calcd. for 586.31 [M+H]⁺ 587.31, found 587.2.

Step 3a. To a solution of trans-methyl 4-[(1R)-1-[3-[4-[2-(2-amino-3-pyridyl)-5-phenyl-imidazo[4,5-b]pyridin-3-yl]phenyl]azetidin-1-yl]ethyl]cyclohexanecarboxylate (40.0 mg, 0.0682 mmol) in THF (1.00 mL) and water (1.00 mL), LiOH·H₂O (8.60 mg, 0.205 mmol) was added and the mixture was stirred at 25° C. for 16 hrs. The mixture was concentrated to remove THF, and the residue was dissolved with DMSO and 1 M HCl, purified by prep-HPLC (Xtimate C18 150*40 mm*5 μm, water(FA)-ACN) to give (1R,4r)-4-((R)-1-(3-(4-(2-(2-aminopyridin-3-yl)-5-phenyl-3H-imidazo[4,5-b]pyridin-3-yl)phenyl)azetidin-1-yl)ethyl)cyclohexane-1-carboxylic acid I-59 (24.8 mg, 63.1% yield) as a yellow solid. ¹H NMR (DMSO-d₆ 400 MHz) δ_{H}=8.26 (d, J=8.4 Hz, 1H), 8.06-7.94 (m, 4H), 7.56-7.34 (m, 7H), 7.20 (dd, J=1.66, 7.6 Hz, 1H), 6.95 (s, 2H), 6.42 (dd, J=4.8, 7.6 Hz, 1H), 3.64 (br s, 3H), 3.12 (br d, J=3.2 Hz, 3H), 2.22 (br d, J 3.2 Hz, 1H), 2.16-2.04 (m, 1H), 1.92 (br d, J=12.4 Hz, 2H), 1.74 (br d, J=11.6 Hz, 1H), 1.51 (br d, J=12.4 Hz, 1H), 1.41-1.18 (m, 3H), 1.16-0.92 (m, 2H), 0.77 (d, J=6.4 Hz, 3H). HPLC Rₜ=3.732 in 8 min chromatography, 0-60AB, purity 99.26%. LCMS Rₜ=2.861 min in 4 min chromatography, Xtimate C18 2.1*30 mm,3 μm, purity 100%, MS ESI calcd. for 572.29 [M+H]⁺ 573.29, found 573.4.

Step 3b. To a solution of trans-methyl 4-[(1S)-1-[3-[4-[2-(2-amino-3-pyridyl)-5-phenyl-imidazo[4,5-b]pyridin-3-yl]phenyl]azetidin-1-yl]ethyl]cyclohexanecarboxylate (50.0 mg, 0.0852 mmol) in THF (1.00 mL) and water (1.00 mL), LiOH·H₂O (11.0 mg, 0.256 mmol) was added and the mixture was stirred at 25° C. for 16 hrs. The mixture was concentrated to remove THF. The residue was dissolved with DMSO and 1M HC, purified by prep-HPLC (Xtimate C18 150*40 mm*5 μm, water(FA)-ACN) to give (1S,4r)-4-((S)-1-(3-(4-(2-(2-aminopyridin-3-yl)-5-phenyl-3H-imidazo[4,5-b]pyridin-3-yl)phenyl)azetidin-1-yl)ethyl)cyclohexane-1-carboxylic acid I-60 (28.8 mg, 59.0% yield) as a yellow solid. ¹H NMR (DMSO-d₆, 400 MHz) δ_{H}=8.25 (d, J=8.4 Hz, 1H), 8.04-7.93 (m, 4H), 7.55-7.49 (m, 21H), 7.49-7.35 (m, 5H), 7.20 (dd, J=1.6, 7.6 Hz, 1H), 6.42 (dd, J=4.8, 7.6 Hz, 1H), 3.82-3.66 (m, 4H), 3.28 (br s, 2H), 2.45-2.36 (m, 1H), 2.17-2.04 (m, 1H), 1.91 (br d, J=11.6 Hz, 2H), 1.70 (br d, J=12.4 Hz, 1H), 1.51 (br d, J=12.4 Hz, 1H), 1.45-1.17 (m, 3H), 1.16-0.92 (m, 2H), 0.80 (d, J=6.4 Hz, 3H). HPLC Rₜ=3.739 in 8 min chromatography, 0-60AB, purity 100%. LCMS Rₜ=2.864 min in 4 min chromatography, Xtimate C18 2.1*30 mm,3 μm, purity 100%, MS ESI calcd. for 572.29 [M+H]⁺ 573.29, found 573.3.

### Example 53 and 54. Synthesis of (1r,4r)-4-((3-(4-(2-(2-aminopyridin-3-yl)-5-phenyl-3H-imidazo[4,5-b]pyridin-3-yl)-2-fluorophenyl)azetidin-1-yl)methyl)cyclohexane-1-carboxylic acid (Compound I-62) and (1s,4s)-4-((3-(4-(2-(2-aminopyridin-3-yl)-5-phenyl-3H-imidazo[4,5-b]pyridin-3-yl)-2-fluorophenyl)azetidin-1-yl)methyl)cyclohexane-1-carboxylic acid (Compound I-63)

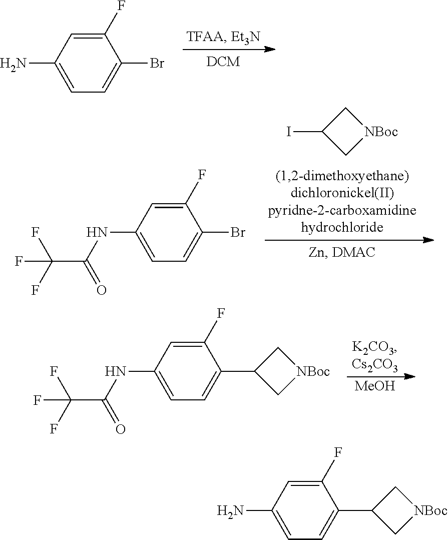

Step 1. To a solution of 4-bromo-3-fluoro-aniline (9.00 g, 47.4 mmol), and TEA (13.0 mL, 94.7 mmol) in DCM (50.0 mL), trifluoroacetic anhydride (11.9 g, 56.8 mmol) was added at 10° C. dropwise, and the mixture subsequently stirred at 20° C. under N₂ for 12 hrs. The reaction mixture was poured into water (500 mL) and extracted with EtOAc (500 mL 3). The combined organic layer was washed with brine (500 mL), dried over Na₂SO₄, filtered, and concentrated to provide N-(4-bromo-3-fluoro-phenyl)-2,2,2-trifluoro-acetamide (15.0 g, 72.0% yield) as a yellow solid. ¹H NMR (400 MHz, CDCl₃) δ=8.28 (s, 1H), 7.64 (dd, J=10.0, 2.4 Hz, 1H), 7.58 (dd, J=8.4, 7.6 Hz, 1H), 7.23 (dd, J=8.8, 1.6 Hz, 1H).

Step 2. A solution of N-(4-bromo-3-fluoro-phenyl)-2,2,2-trifluoro-acetamide (7.00 g, 24.5 mmol), tert-butyl 3-iodoazetidine-1-carboxylate (7.62 g, 26.9 mmol), Zn (9.39 g, 144 mmol), (1,2-dimethoxyethane)dichloronickel(II) (1.08 g, 4.89 mmol) and pyridine-2-carboxamidine hydrochloride (771 mg, 4.89 mmol) in DMA (70.0 ml) was stirred at 100° C. under N₂ for 4 hrs. The reaction was filtered, the filtrate was poured into water (200 mL) and the whole extracted with EtOAc (500 mL×5). The combined organic layer was washed with brine (200 mL), dried over Na₂SO₄, filtered, and concentrated. The residue was purified by column chromatography (SiO₂, 20-25% EtOAc in PE) to provide tert-butyl 3-[2-fluoro-4-[(2,2,2-trifluoroacetyl)amino]phenyl]azetidine-1-carboxylate (2.10 g, 23.7% yield) as a white solid. ¹H NMR (400 MHz, CDCl₃) δ=8.06 (s, 1H), 7.55 (dd, J=11.2, 2.0 Hz, 1H), 7.41 (s, 1H), 7.26 (d, J=2.0 Hz, 1H), 4.31-4.40 (m, 2H), 4.02 (d, J=7.2 Hz, 3H), 1.49 (s, 9H).

Step 3. A solution of tert-butyl 3-[2-fluoro-4-[(2,2,2-trifluoroacetyl)amino]phenyl]azetidine-1-carboxylate (2.00 g, 5.52 mmol), K₂CO₃ (2.29 g, 16.6 mmol) and Cs₂CO₃ (1.81 g, 5.58 mmol) in methanol (20.0 mL) was stirred at 60° C. under N₂ for 12 hrs. The reaction mixture was poured into water (100 mL) and extracted with EtOAc (150 mL×3). The combined organic layer was washed with brine (50 mL), dried over Na₂SO₄, filtered and concentrated to provide tert-butyl 3-(4-amino-2-fluoro-phenyl)azetidine-1-carboxylate (1.40 g, 73.3% yield) as a yellow oil. ¹H NMR (400 MHz, CDCl₃) δ=7.14 (t, J=8.4 Hz, 1H), 6.52 (dd, J=7.6, 1.8 Hz, 1H), 6.43 (dd, J=11.6, 2.0 Hz, 1H), 4.25-4.33 (m, 2H), 3.99 (t, J1=7.2 Hz, 2H), 3.83-3.93 (m, 1H), 1.48 (s, 9H).

### Synthesis of (1r,4r)-4-((3-(4-(2-(2-aminopyridin-3-yl)-5-phenyl-3H-imidazo[4,5-b]pyridin-3-yl)-2-fluorophenyl)azetidin-1-yl)methyl)cyclohexane-1-carboxylic acid (Compound I-62) and (1s,4s)-4-((3-(4-(2-(2-aminopyridin-3-yl)-5-phenyl-3H-imidazo[4,5-b]pyridin-3-yl)-2-fluorophenyl)azetidin-1-yl)methyl)cyclohexane-1-carboxylic acid (Compound I-63)

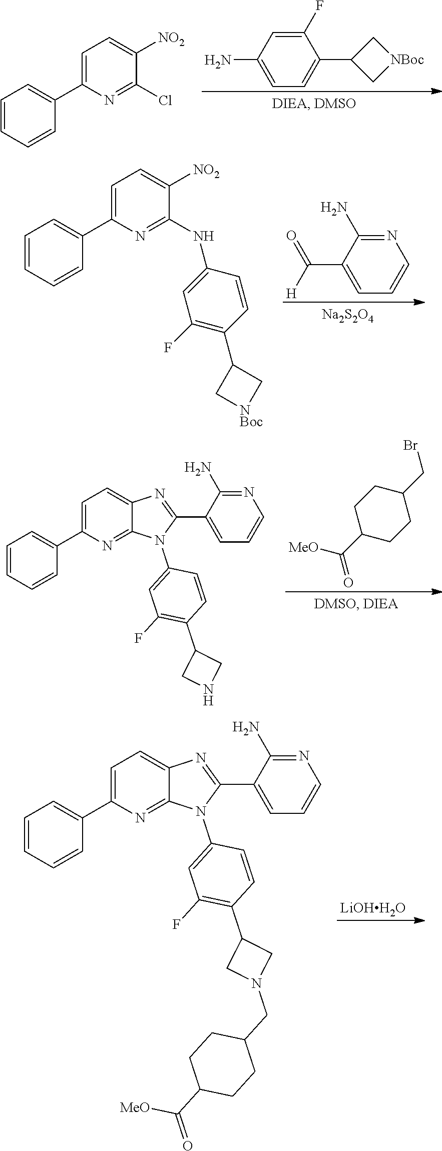

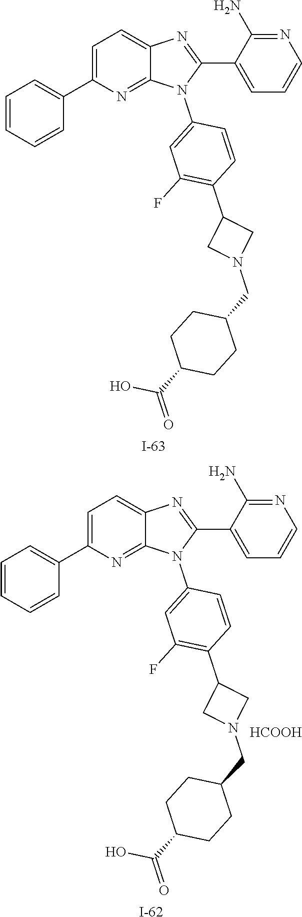

Step 1. To a solution of tert-butyl 3-(4-amino-2-fluoro-phenyl)azetidine-1-carboxylate (1.36 g, 5.11 mmol) in DMSO (10.0 mL), 2-chloro-3-nitro-6-phenyl-pyridine (1.20 g, 5.11 mmol) and DIEA (4.20 mL, 25.6 mmol) were added under N₂ atmosphere and the mixture was stirred at 20° C. for 6 hrs. The reaction was taken up in EtOAc (100 mL), and the organics were washed with water (50 mL) and brine (3×50 mL). The organics were then separated and dried (Na₂SO₄) before concentration to dryness. The crude was then purified by flash column chromatography eluting with 30% of EtOAc in isohexane to give tert-butyl 3-[2-fluoro-4-[(3-nitro-6-phenyl-2-pyridyl)amino]phenyl]azetidine-1-carboxylate (700 mg, 29.2% yield) as a red solid. ¹H NMR (400 MHz, DMSO-d₆) δ=10.20 (s, 1H), 8.68 (d, J=8.8 Hz, 1H), 8.09-8.21 (m, 2H), 7.84 (dd, J=12.8, 1.6 Hz, 1H), 7.69 (d, J=8.8 Hz, 1H), 7.59-7.64 (m, 3H), 7.49-7.57 (m, 1H), 4.30 (s, 2H), 4.11 (s, 1H), 3.94-4.08 (m, 3H), 1.45 (s, 9H).

Step 2. To a solution of tert-butyl 3-[2-fluoro-4-[(3-nitro-6-phenyl-2-pyridyl)amino]phenyl]azetidine-1-carboxylate (600 mg, 1.29 mmol) in DMSO (5.00 mL) and methanol (1.00 mL), 2-aminopyridine-3-carbaldehyde (316 mg, 2.58 mmol) and Na₂S₂O₄ (1.35 g, 7.75 mmol) were added at 25° C. and the mixture was stirred at 100° C. for 10 hrs. The reaction was taken up in EtOAc (100 mL), the organics were washed with water (50 mL) and then brine (3×50 mL). The organics were then separated and dried (Na₂SO₄) before concentration to dryness. The crude was purified by flash column chromatography eluting with 100% EtOAc in Isohexane. The residue was further purified by prep-HPLC (Column: Welch Xtimate C18 40*200 mm 7 μm; mobile phase: [water(FA)-ACN]; B %: 8%-48%; 25 min) to give 3-[3-[4-(azetidin-3-yl)-3-fluoro-phenyl]-5-phenyl-imidazo[4,5-b]pyridin-2-yl]pyridin-2-amine (3.00 g, crude) as a yellow solid. ¹H NMR (400 MHz, DMSO-d₆) δ=8.35-8.43 (m, 2H), 8.32 (d, J=8.4 Hz, 1H), 8.02-8.11 (m, 4H), 7.68-7.75 (m, 1H), 7.53 (br t, 0.1=8.0 Hz, 3H), 7.46 (d, J=7.6 Hz, 1H), 7.37-7.42 (m, 1H), 7.34 (dd, J=7.6, 1.63 Hz, 1H), 6.87 (s, 1H), 6.53 (dd, J=7.6, 4.8 Hz, 1H), 4.24-4.36 (m, 1H), 3.99-4.11 (m, 4H).

Step 3. To a solution of 3-[3-[4-(azetidin-3-yl)-3-fluoro-phenyl]-5-phenyl-imidazo[4,5-b]pyridin-2-yl]pyridin-2-amine (100 mg, 0.229 mmol) in DMSO (2.00 mL), DIEA (0.200 mL, 1.15 mmol) and methyl 4-(bromomethyl)cyclohexanecarboxylate (81.0 mg, 0.344 mmol) were added and the mixture was stirred at 25° C. for 16 hrs. The reaction was taken up in EtOAc (30 mL) and the organics were washed with brine (3×50 mL). The organics were then separated and dried (Na₂SO₄) before concentration to dryness. Methyl 4-[[3-[4-[2-(2-amino-3-pyridyl)-5-phenyl-imidazo[4,5-b]pyridin-3-yl]-2-fluoro-phenyl]azetidin-1-yl]methyl]cyclohexanecarboxylate (150 mg, 66.5% yield) was obtained as a white solid. LCMS Rₜ=0.537 min in 1 min chromatography, Agilent PoroShell 120 EC-C18 2.7 μm 3.0*50 mm, purity 88.4%, MS ESI calcd. for 590.28 [M+H]⁺ 591.28, found 591.3.

Step 4. To a solution of methyl 4-[[3-[4-[2-(2-amino-3-pyridyl)-5-phenyl-imidazo[4,5-b]pyridin-3-yl]-2-fluoro-phenyl]azetidin-1-yl]methyl]cyclohexanecarboxylate (150 mg, 0.254 mmol) in water (1.00 mL) and THF (3.00 mL), LiOH·H₂O (53.0 mg, 1.27 mmol) was added under N₂ atmosphere and the mixture was stirred at 40° C. for 2 hrs. The reaction mixture was concentrated and purified by prep-HPLC (column: Welch Xtimate C18 200*40 mm*7 μm; mobile phase: [water(FA)-ACN];B %: 0%-6-36%, 20 min) to give cis-4-[[3-[4-[2-(2-amino-3-pyridyl)-5-phenyl-imidazo[4,5-b]pyridin-3-yl]-2-fluoro-phenyl]azetidin-1-yl]methyl]cyclohexanecarboxylic acid I-63 (20.0 mg, 13.8% yield) as a white solid and trans-4-[[3-[4-[2-(2-amino-3-pyridyl)-5-phenyl-imidazo[4,5-b]pyridin-3-yl]-2-fluoro-phenyl]azetidin-1-yl]methyl]cyclohexanecarboxylic acid 1-62 (11.0 mg, 7.44% yield) as a white solid.

(1s,4s)-4-((3-(4-(2-(2-aminopyridin-3-yl)-5-phenyl-3H-imidazo[4,5-b]pyridin-3-yl)-2-fluorophenyl)azetidin-1-yl)methyl)cyclohexane-1-carboxylic acid I-63: ¹H NMR (400 MHz, DMSO-d₆) δ=8.27 (d, J=8.0 Hz, 1H), 7.92-8.08 (m, 4H), 7.61 (t, J=8.4 Hz, 1H), 7.38-7.51 (m, 4H), 7.26-7.32 (m, 2H), 6.84 (s, 2H), 6.48 (dd, J=7.6, 5.2 Hz, 1H), 3.79-3.87 (m, 1H), 3.67 (t, J=7.2 Hz, 2H), 3.12 (t, J=6.8 Hz, 2H), 2.28 (d, J=6.4 Hz, 2H), 2.05-2.16 (m, 1H), 1.84-1.91 (m, 2H), 1.79 (d, J=13.2 Hz, 2H), 1.23-1.29 (m, 3H), 0.84-0.94 (m, 2H). HPLC Rₜ=2.331 min in 8 min chromatography, ACE Excel 3 C18 4.6*100 mm, purity 99.8%. LCMS Rₜ=1.535 min in 4 min chromatography, Agilent PoroShell 120 EC-C18 2.7 μm 3.0*50 mm, purity 99.7%, MS ESI calcd. for 576.26 [M+H]⁺ 577.26, found 577.3.

(1r,4r)-4-((3-(4-(2-(2-aminopyridin-3-yl)-5-phenyl-3H-imidazo[4,5-b]pyridin-3-yl)-2-fluorophenyl)azetidin-1-yl)methyl)cyclohexane-1-carboxylic acid I-62: ¹H NMR (400 MHz, DMSO-d₆) δ=8.27 (d, J=8.4 Hz, 1H), 8.17 (s, 1H), 7.97-8.06 (m, 4H), 7.60 (t, J=8.4 Hz, 1H), 7.38-7.51 (m, 4H), 7.25-7.32 (m, 2H), 6.84 (s, 2H), 6.48 (dd, J=7.2, 4.8 Hz, 1H), 3.80-3.88 (m, 1H), 3.68 (t, J=7.2 Hz, 2H), 3.13 (d, J=7.2 Hz, 2H), 2.42 (s, 1H), 2.31 (s, 1H), 1.77-1.87 (m, 2H), 1.39-1.57 (m, 5H), 1.12-1.27 (m, 3H). HPLC Rₜ=2.132 min in 8 min chromatography, ACE Excel 3 C18 4.6*100 mm, purity 99.9%. LCMS Rₜ=1.461 min in 4 min chromatography, Agilent PoroShell 120 EC-C18 2.7 μm 3.0*50 mm, purity 100%, MS ESI calcd. for 576.26 [M+H]⁺ 577.26, found 577.2.

### Example 55. Synthesis of 4-((3-(5-(2-(2-aminopyridin-3-yl)-5-phenyl-3H-imidazo[4,5-b]pyridin-3-yl)pyridin-2-yl)azetidin-1-yl)methyl)cyclohexane-1-carboxylic acid (Compound I-64)

### Synthesis of tert-butyl 3-(5-aminopyridin-2-yl)azetidine-1-carboxylate

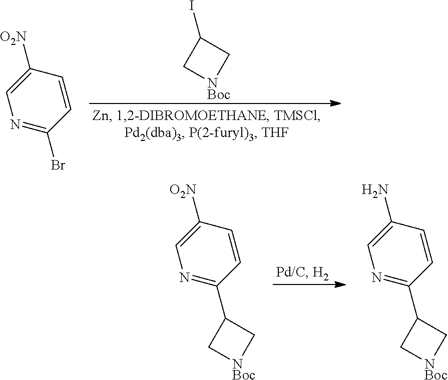 [SMILES: O=[N+]([O-])c1ccc(Br)nc1 ; CC(C)(C)OC(=O)N1CC(I)C1 ; CC(C)(C)OC(=O)N1CC(c2ccc([N+](=O)[O-])cn2)C1 ; CC(C)(C)OC(=O)N1CC(c2ccc(N)cn2)C1]

Step 1. Zn (3.49 g, 53.7 mmol) was vigorously stirred in THF (24.0 mL) under nitrogen and 1,2-dibromoethane (1.16 g, 6.20 mmol) was added. The suspension was then heated at 80° C. for 8 mins and subsequently allowed to cool to room temperature. TMSCl (673 mg, 6.20 mmol) in THF (12.0 mL) was then added and the mixture further stirred at room temperature for 45 mins. A solution of tert-butyl 3-iodoazetidine-1-carboxylate (11.7 g, 41.3 mmol) in THF (24.0 mL) was then added dropwise to the solution over a period of 15 mins, and the reaction mixture stirred at room temperature for 2 hrs Pd₂(dba)₃ (757 mg, 0.827 mmol) and P(2-furyl)₃ (576 mg, 2.48 mmol) were then added to the mixture, followed by 2-bromo-5-nitro-pyridine (9.20 g, 45.5 mmol) in THF (48.0 mL). The mixture was then heated at 55° C. for 3 hrs, cooled to room temperature and quenched with brine (200 mL). Extraction with DCM (300 mL×3), drying (Na₂SO₄) of the organic phase, filtration and evaporation in vacuo provided the crude material, which was purified by flash column chromatography (heptane:ethyl acetate, 3:1) to give tert-butyl 3-(5-nitro-2-pyridyl)azetidine-1-carboxylate (8.62 g, 74.7% yield) as a brown oil. ¹H NMR (400 MHz, DMSO-d₆) δ=9.39 (d, J=2.4 Hz, 1H), 8.55 (dd, J=8.4, 2.4 Hz, 1H), 7.64 (d, J=8.4 Hz, 1H), 4.22 (d, J=7.2 Hz, 2H), 4.11 (dd, J=8.4, 5.6 Hz, 1H), 4.00-4.06 (m, 2H), 1.39 (s, 91H).

Step 2. To a solution of tert-butyl 3-(5-nitro-2-pyridyl)azetidine-1-carboxylate (8.60 g, 30.8 mmol) in THF (80.0 mL), wet Pd/C (10%, 2.00 g) was added under N₂ atmosphere. The suspension was degassed and purged with H₂ (×3), and the reaction mixture was stirred under H₂ (50 psi) at 50° C. for 16 hrs. The mixture was filtered via a celite pad, and the filter cake was washed with DCM:MeOH=1:1 (300 mL). The organic phase was concentrated to give tert-butyl 3-(5-amino-2-pyridyl) azetidine-1-carboxylate (6.20 g, 24.9 mmol) as a yellow oil ¹H NMR (400 MHz, DMSO-d₆) δ=7.99 (d, J=2.4 Hz, 1H), 6.95-7.01 (m, 1H), 6.88-6.92 (m, 1H), 5.25 (br s, 1H), 4.12 (s, 1H), 3.94 (s, 2H), 3.66-3.79 (m, 1H), 1.43 (s, 9H).

### Synthesis of 4-((3-(5-(2-(2-aminopyridin-3-yl)-5-phenyl-3H-imidazo[4,5-b]pyridin-3-yl)pyridin-2-yl)azetidin-1-yl)methyl)cyclohexane-1-carboxylic acid (Compound I-64)

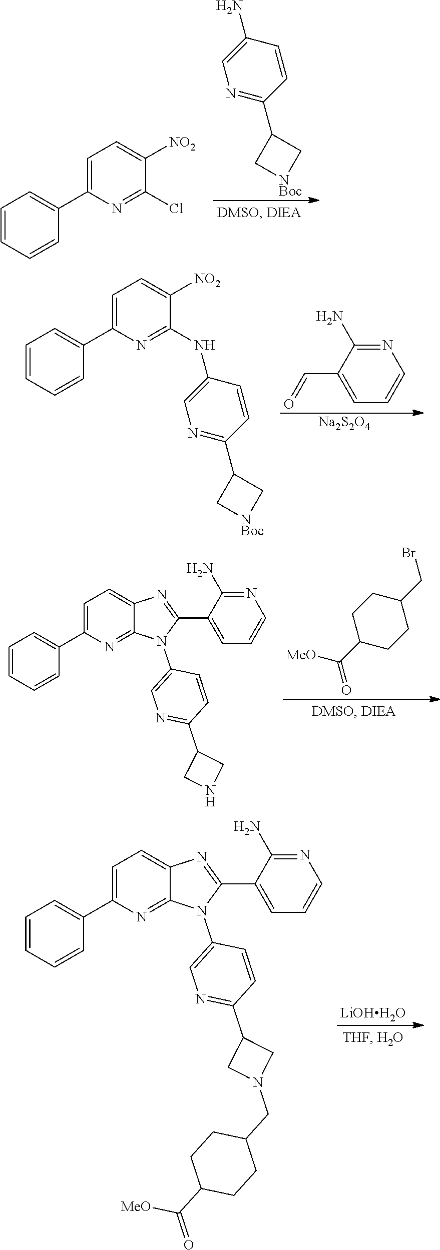

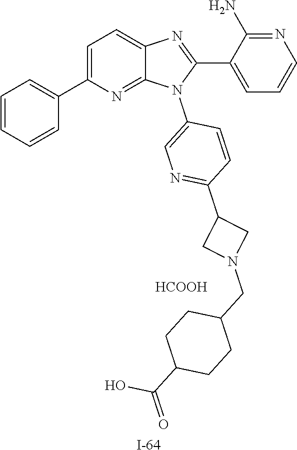

Step 1. To a solution of tert-butyl 3-(5-amino-2-pyridyl)azetidine-1-carboxylate (5.70 g 22.9 mmol) in DMSO (60.0 mL), 2-chloro-3-nitro-6-phenyl-pyridine (6.40 g, 27.3 mmol) and DIEA (19.0 mL, 114 mmol) were added under N₂ atmosphere. The mixture was stirred at 100° C. for 6 hrs. The reaction was taken up in EtOAc (500 mL) and the organics washed with water (100 mL) and a saturated brine solution (3×100 mL). The organics were then separated and dried with Na₂SO₄ before being concentrated to dryness. The crude was then purified by flash column chromatography eluting with 30% of EtOAc in isohexane to give tert-butyl 3-[5-[(3-nitro-6-phenyl-2-pyridyl)amino]-2-pyridyl]azetidine-1-carboxylate (6.20 g, 55.2% yield) as a red solid. ¹H NMR (400 MHz, DMSO-d₆) δ=10.14 (s, 1H), 8.92 (d, J=2.4 Hz, 1H), 8.64 (d, 0.1=8.4 Hz, 1H), 8.15 (dd, J=8.4, 2.4 Hz, 1H), 8.01-8.10 (m, 2H), 7.63 (d, J-=8.4 Hz, 1H), 7.51-7.56 (m, 3H), 7.41 (d, 0.1=8.4 Hz, 1H), 4.15-4.28 (m, 2H), 4.00-4.08 (m, 2H), 3.90-3.99 (m, 1H), 1.42 (s, 9H).

Step 2. To a solution of tert-butyl 3-[5-[(3-nitro-6-phenyl-2-pyridyl)amino]-2-pyridyl]azetidine-1-carboxylate (5.80 g, 13.0 mmol) in DMSO (50.0 mL) and methanol (10.0 mL), 2-aminopyridine-3-carbaldehyde (3.16 g, 25.9 mmol) and Na₂S₂O₄ (13.5 g, 77.8 mmol) were added at 25° C. and the mixture was stirred at 100° C. for 10 hrs. The crude product was purified by prep-HPLC (column: Phenomenex Luna C18 250*80 mm*10 μm; mobile phase: [water (FA)-ACN]; B %: 0%-30%, 15 min) to provide 3-[3-[6-(azetidin-3-yl)-3-pyridyl]-5-phenyl-imidazo[4,5-b]pyridin-2-yl]pyridin-2-amine (2.50 g, 46.0% yield) as a brown solid. ¹H NMR (400 MHz, DMSO-d₆) δ=8.78 (d, J=2.0 Hz, 1H), 8.35 (s, 1H), 8.30 (d, J=8.4 Hz, 1H), 8.00-8.06 (m, 4H), 7.96 (dd, J=8.4, 2.4 Hz, 1H), 7.57 (d, J=8.0 Hz, 1H), 7.45-7.50 (m, 2H), 7.38-7.43 (m, 1H), 7.35 (dd, J=7.6, 1.6 Hz, 1H), 6.72 (s, 2H), 6.50 (dd, J=7.6, 4.8 Hz, 1H), 4.27 (br d, 0.1=8.4 Hz, 1H), 4.14-4.19 (m, 4H).

Step 3. To a solution of 3-[3-[6-(azetidin-3-yl)-3-pyridyl]-5-phenyl-imidazo[4,5-b]pyridin-2-yl]pyridin-2-amine (220 mg, 0.524 mmol) in DMSO (3.00 mL), DIEA (0.470 mL, 2.62 mmol) and methyl 4-(bromomethyl)cyclohexanecarboxylate (185 mg, 0.787 mmol) were added and the mixture was stirred at 25° C. for 16 hrs. The reaction mixture was diluted with water (3 mL) and filtered. The filter pad was washed with ACN and concentrated in vacuo to give methyl 4-[[3-[5-[2-(2-amino-3-pyridyl)-5-phenyl-imidazo[4,5-b]pyridin-3-yl]-2-pyridyl]azetidin-1-yl]methyl]cyclohexanecarboxylate (350 mg, 69.8% yield) as a white solid. ¹H NMR (400 MHz, DMSO-d₆) δ ppm 8.66 (d, J=2.4 Hz, 1H), 8.28 (d, J=8.4 Hz, 1H), 8.21 (s, 1H), 7.99-8.05 (m, 4H), 7.55 (d, J=8.8 Hz, 1H), 7.45-7.50 (m, 2H), 7.41 (d, 0.1=7.2 Hz, 1H), 7.32 (dd, J=7.6, 1.6 Hz, 1H), 6.72 (s, 2H), 6.49 (dd, J=7.6, 4.8 Hz, 1H), 3.89-4.00 (m, 2H), 3.55-3.61 (m, 6H), 1.83-1.95 (m, 4H), 1.46-1.59 (m, 4H), 1.22-1.33 (m, 4H).

Step 4. To a solution of methyl 4-[[3-[5-[2-(2-amino-3-pyridyl)-5-phenyl-imidazo[4,5-b]pyridin-3-yl]-2-pyridyl]azetidin-1-yl]methyl]cyclohexanecarboxylate (350 mg, 0.610 mmol) in water (1.00 mL) and THF (3.00 mL), LiOH·H₂O (128 mg, 3.05 mmol) was added under N₂ atmosphere and the mixture was stirred at 40° C. for 2 hrs. The crude product was purified by prep-HPLC (column. Welch Xtimate C18 200*40 mm*7 μm; mobile phase: [water (FA)-ACN], B %: 0%-34%, 20 min) to give 4-[[3-[5-[2-(2-amino-3-pyridyl)-5-phenyl-imidazo[4,5-b]pyridin-3-yl]-2-pyridyl]azetidin-1-yl]methyl]cyclohexanecarboxylic acid I-64 (30.0 mg, 8.71% yield) as a white solid. ¹H NMR (400 MHz, DMSO-d₆) δ=8.67 (d, J=2.0 Hz, 1H), 8.29 (d, 0.1=8.4 Hz, 1H), 8.17 (s, 1H), 7.97-8.07 (m, 4H), 7.91 (dd, J=8.4, 2.4 Hz, 1H), 7.56 (d, J=8.4 Hz, 1H), 7.45-7.52 (m, 2H), 7.42 (d, J-=7.2 Hz, 1H), 7.33 (d, J=7.6 Hz, 1H), 6.73 (s, 2H), 6.50 (dd, t=7.6, 4.8 Hz, 1H), 3.84 (d, J=7.6 Hz, 1H), 3.67 (t, J=7.2 Hz, 2H), 3.32 (s, 2H), 2.33 (d, J=6.4 Hz, 2H), 2.10 (s, 1H), 1.87 (d, J=11.2 Hz, 2H), 1.78 (d, J=10.8 Hz, 2H), 1.27 (dd, J=120, 2.4 Hz, 3H), 0.90 (dd, J=12.0, 1.6 Hz, 2H) HPLC Rₜ=2.098n in 8 min chromatography, ACE Excel 3 C18 4.6*100 mm, purity 99.51 LCMS Rₜ=1.444 min in 4 min chromatography, Agilent PoroShell 120 EC-C18 2.7 um 3.0*50 mm, purity 99.94%, MS ESI calcd. for 559.27 [M+H]⁺ 560.27, found 560.3.

### Example 56. Synthesis of (1r,4r)-4-((3-(4-(2-(2-aminopyridin-3-yl)-6-phenyl-3H-imidazo[4,5-b]pyridin-3-yl)phenyl)azetidin-1-yl)methyl)cyclohexane-1-carboxylic acid (Compound I-65)

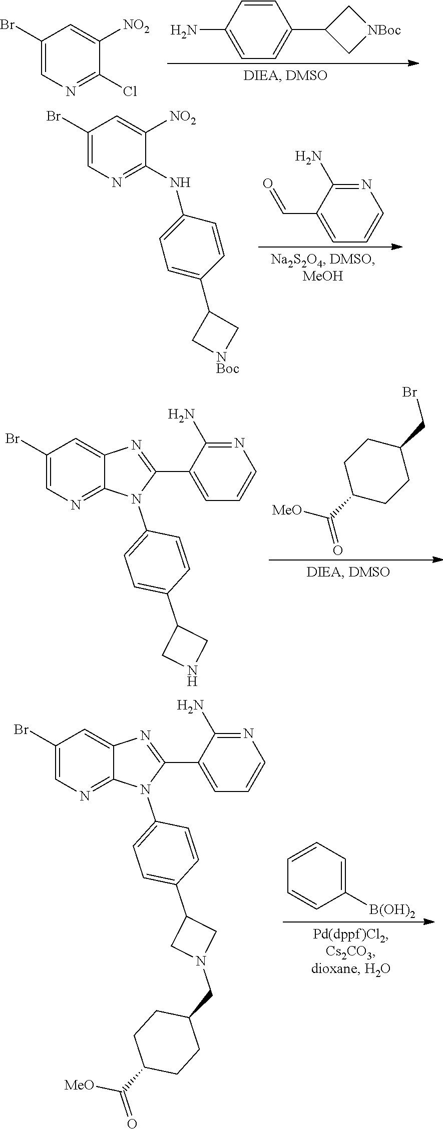

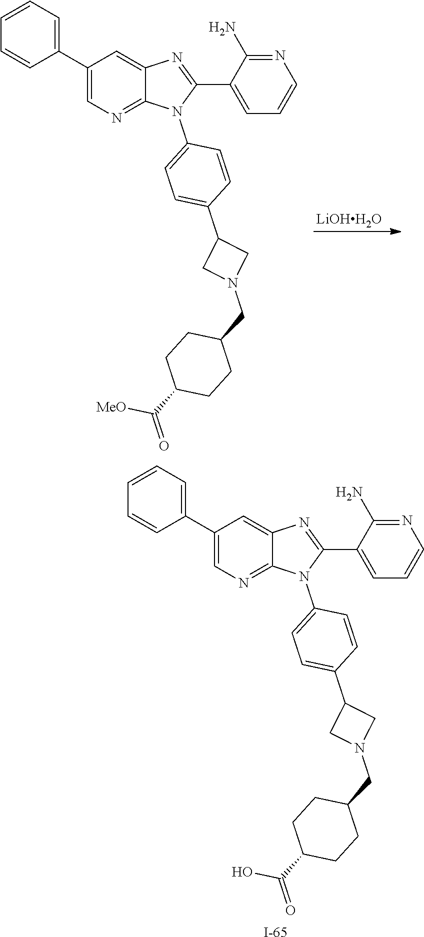

Step 1. To a solution of 5-bromo-2-chloro-3-nitro-pyridine (1.91 g, 8.05 mmol) in DMSO (20.0 mL), tert-butyl 3-(4-aminophenyl)azetidine-1-carboxylate (2.00 g, 8.05 mmol) and DIEA (4.20 mL, 24.2 mmol) were added and the reaction stirred at 100° C. for 16 hrs. Water (200 mL) was added to the reaction mixture and the whole extracted with EtOAc (100 mL×3). The organic layer was dried over Na₂SO₄ and concentrated in vacuo to provide the crude material. The crude was purified by flash column (0-50% of EtOAc in PE) to give tert-butyl 3-[4-[(5-bromo-3-nitro-2-pyridyl)amino]phenyl]azetidine-1-carboxylate (3.07 g, 84.8% yield) as a red solid. ¹H NMR (400 MHz, DMSO-d₆) δ=9.94 (s, 1H), 8.67 (d, J=2.4 Hz, 1H), 8.59 (d, J=2.4 Hz, 1H), 7.56 (d, J=8.4 Hz, 2H), 7.33 (d, J=8.4 Hz, 2H), 4.25 (t, J=7.6 Hz, 2H), 3.87-3.75 (m, 3H), 1.41 (s, 9H).

Step 2. To a solution of tert-butyl 3-[4-[(5-bromo-3-nitro-2-pyridyl)amino]phenyl]azetidine-1-carboxylate (2.70 g, 6.01 mmol) in DMSO (3.00 mL) and methanol (4.00 mL), 2-aminopyridine-3-carbaldehyde (1.03 g, 8.41 mmol) and Na₂S₂O₄ (4.18 g, 24.0 mmol) were added and the reaction was stirred at 100° C. for 16 hrs. Water (300 mL) was then added and the whole was extracted with EtOAc (200 mL×3). The organic layer was dried over Na₂SO₄ and concentrated in vacuo to provide the crude material. The aqueous layer was purified by prep-HPLC (column: Welch Xtimate C18 150*40 mm*5 um; mobile phase: water (FA)-ACN; B %: NH₂%, 60 min) to give 3-[3-[4-(azetidin-3-yl)phenyl]-6-bromo-imidazo[4,5-b]pyridin-2-yl]pyridin-2-amine (600 mg, 23.7% yield) as white solid. ¹H NMR (400 MHz, DMSO-d₆) δ=8.55-8.48 (m, 1H), 8.41 (d, J=2.0 Hz, 1H), 8.00 (dd, J=1.6, 4.8 Hz, 1H), 7.55-7.42 (m, 2H), 7.41-7.34 (m, 2H), 7.23 (d, J=8.0 Hz, 1H), 7.01-6.87 (m, 2H), 6.46-6.37 (m, 1H), 3.98-3.70 (m, 3H), 3.63-3.56 (m, 2H).

Step 3. To a solution of 3-[3-[4-(azetidin-3-yl)phenyl]-6-bromo-imidazo[4,5-b]pyridin-2-yl]pyridin-2-amine (300 mg, 0.712 mmol) in DMSO (3.00 mL), trans-methyl 4-(bromomethyl)cyclohexanecarboxylate (234 mg, 0.997 mmol) and DIEA were added and the reaction was stirred at 80° C. for 16 hrs. Water (30 mL) was added to the reaction mixture and the whole extracted with EtOAc (20 mL×3). The organic layer was dried over Na₂SO₄ and concentrated in vacuo to provide the crude material. The crude was purified by flash column (0-30% of MeOH in DCM) to give trans-methyl 4-[[3-[4-[2-(2-amino-3-pyridyl)-6-bromo-imidazo[4,5-b]pyridin-3-yl]phenyl]azetidin-1-yl]methyl]cyclohexanecarboxylate (100 mg, 24.4% yield) as a yellow solid. ¹H NMR (400 MHz, DMSO-d₆) δ=8.50 (d, J=2.0 Hz, 1H), 8.41 (d, J=2.0 Hz, 1H), 8.00 (dd, J=2.0, 4.8 Hz, 1H), 7.48 (d, J=8.4 Hz, 2H), 7.37 (d, J=8.4 Hz, 2H), 7.24 (dd, J=2.0, 7.6 Hz, 1H), 6.92 (s, 2H), 6.42 (dd, J=4.8, 7.6 Hz, 1H), 3.57 (s, 6H), 3.10 (s, 1H), 2.54 (s, 1H), 2.34-2.24 (m, 2H), 1.92-1.70 (m, 4H), 1.36-1.20 (m, 4H), 1.01-0.82 (m, 2H).

Step 4. To a solution of trans-methyl 4-[[3-[4-[2-(2-amino-3-pyridyl)-6-bromo-imidazo[4,5-b]pyridin-3-yl]phenyl]azetidin-1-yl]methyl]cyclohexanecarboxylate (80.0 mg, 0.139 mmol) in 1,4-dioxane (3.00 mL) and water (1.00 mL), phenylboronic acid (20.0 mg, 0.167 mmol), Pd(dppf)Cl₂ (10.0 mg, 0.0139 mmol) and Cs₂CO₃ (136 mg, 0.417 mmol) were added at 25° C., and the mixture was stirred at 90° C. for 14 hrs under N₂. Water (20 mL) was added to the reaction mixture and the whole was extracted with EtOAc (15 mL×3). The organic layer was dried over Na₂SO₄ and concentrated in vacuo to provide the crude material. The crude was purified by flash column (0-30% of MeOH in DCM) to give trans-methyl 4-[[3-[4-[2-(2-amino-3-pyridyl)-6-phenyl-imidazo[4,5-b]pyridin-3-yl]phenyl]azetidin-1-yl]methyl]cyclohexanecarboxylate (40.0 mg, 50.2% yield) as yellow oil. LCMS Rₜ=0.809 min in 1.5 min chromatography, purity 67.3%, MS ESI calcd. For 572.29 [M+H]⁺ 573.29, found 573.5.

Step 5. To a solution of trans-methyl 4-[[3-[4-[2-(2-amino-3-pyridyl)-6-phenyl-imidazo[4,5-b]pyridin-3-yl]phenyl]azetidin-1-yl]methyl]cyclohexanecarboxylate (40.0 mg, 0.0698 mmol) in THF (0.500 mL) and water (0.500 mL), LiOH·H₂O (12.0 mg, 0.279 mmol) was added and the reaction stirred at 25° C. for 16 hrs. The reaction mixture was purified by prep-HPLC (column: Welch Xtimate C18 150*40 mm*5 um; mobile phase: water (FA)-ACN; B %:5%-35%, 60 min) to give trans-4-[[3-[4-[2-(2-amino-3-pyridyl)-6-phenyl-imidazo[4,5-b]pyridin-3-yl]phenyl]azetidin-1-yl]methyl]cyclohexanecarboxylic acid 1-65 (13.0 mg, 33.1% yield) as a yellow solid. LCMS Rₜ=2.760 min in 4 min chromatography, purity 100%, MS ESI calcd. For 558.27 [M+H]⁺ 559.27, found 559.1. HPLC Rₜ=3.502 min in 8 min chromatography, purity 99.2% ¹H NMR (400 MHz, DMSO-d₆) δ=8.62 (d, J=2.0 Hz, 1H), 8.46 (d, J=2.0 Hz, 1H), 8.00 (dd, J=1.6, 4.4 Hz, 1H), 7.78 (d, J=7.6 Hz, 2H), 7.55-7.48 (m, 4H), 7.45-7.38 (m, 3H), 7.25 (dd, J=1.6, 7.6 Hz, 1H), 7.02 (s, 2H), 6.42 (dd, J=5.2, 7.6 Hz, 1H), 3.71-3.58 (m, 3H), 3.14-3.10 (m, 2H), 2.30 (s, 1H), 2.15-2.05 (m, 1H), 1.91-1.76 (m, 4H), 1.34-1.21 (m, 4H), 0.96-0.84 (m, 2H).

### Example 57. Synthesis of (1r,4r)-4-((3-(4-(2-(2-aminopyridin-3-yl)-5-(4-fluorophenyl)-3H-imidazo[4,5-b]pyridin-3-yl)phenyl)azetidin-1-yl)methyl)cyclohexane-1-carboxylic acid (Compound I-66)

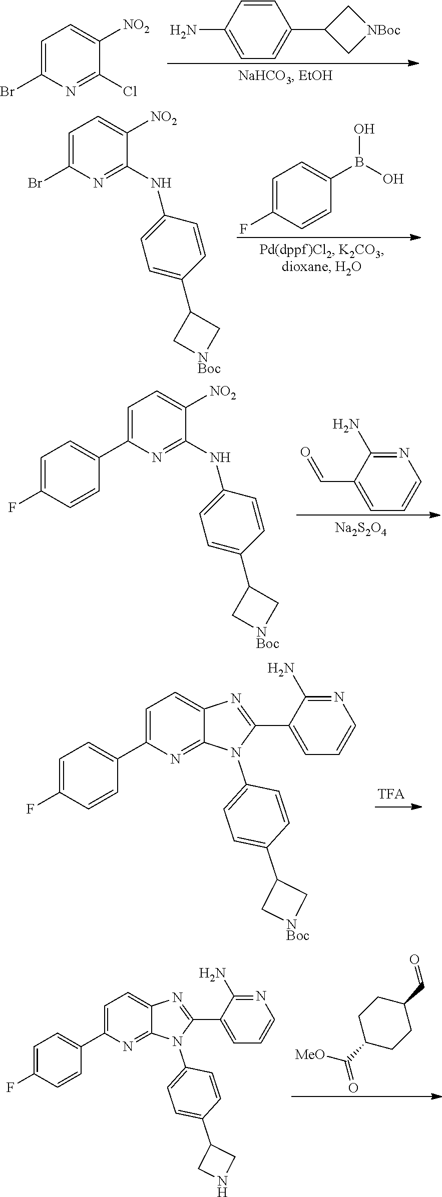

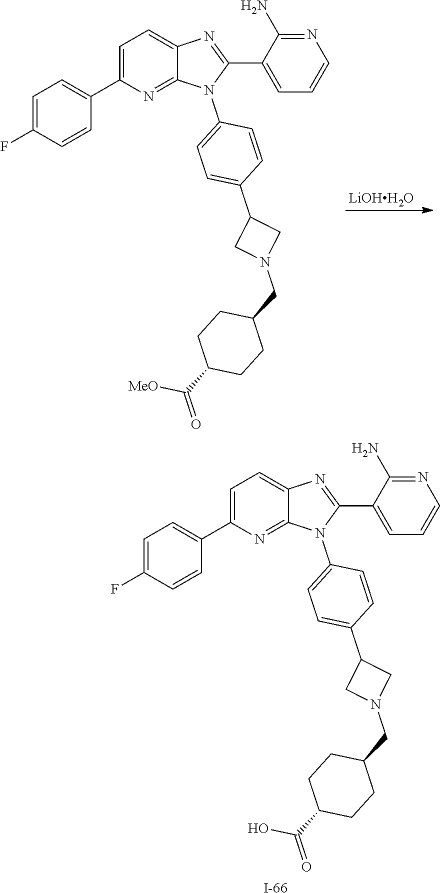

Step 1. To a solution of 6-bromo-2-chloro-3-nitro-pyridine (1.83 g, 7.71 mmol) in ethanol (20.0 mL), tert-butyl 3-(4-aminophenyl)azetidine-1-carboxylate (1.91 g, 7.71 mmol) and NaHCO₃ (2.34 g, 23.1 mmol) were added at 25° C. under N₂, and the mixture was stirred for 16 hrs water (20 mL) was added, and the mixture was filtered. The wet cake was subsequently concentrated under reduced pressure to provide the crude material. The crude was purified by flash chromatography eluting with PE/EtOAc (from 0% to 50% of EtOAc) to give tert-butyl 3-[4-[(6-bromo-3-nitro-2-pyridyl)amino]phenyl]azetidine-1-carboxylate (1.50 g, 43.3% yield) as a red oil. ¹H NMR (400 MHz, DMSO-d₆) δ=10.09 (s, 1H), 8.39 (d, J=8.4 Hz, 1H), 7.60-7.56 (m, 2H), 7.36 (d, J=8.4 Hz, 2H), 7.13 (d, J=8.6 Hz, 1H), 4.25 (br s, 2H), 3.87-3.76 (m, 3H), 1.41 (s, 9H).

Step 2. To a solution of tert-butyl 3-[4-[(6-bromo-3-nitro-2-pyridyl)amino]phenyl]azetidine-1-carboxylate (950 mg, 2.11 mmol) in 1,4-dioxane (12.0 mL) and water (3.00 mL), (4-fluorophenyl)boronic acid (355 mg, 2.54 mmol), K₂CO₃ (731 mg, 5.29 mmol) and Pd(dppf)Cl₂ (155 mg, 0.211 mmol) were added at 25° C. under N₂, and the mixture was stirred at 80° C. for 16 hrs. EtOAc (50 mL) was added, and the mixture was filtered. Water (250 mL) was added to the filtrate, and the mixture was extracted with EtOAc (250 mL×3). The organic phase was washed with brine (500 mL), dried over Na₂SO₄, filtered, and concentrated under reduced pressure to give a crude. The crude was purified by flash chromatography eluting with PE/EtOAc (from 0% to 30% of EtOAc in PE) to give tert-butyl 3-[4-[[6-(4-fluorophenyl)-3-nitro-2-pyridyl]amino]phenyl]azetidine-1-carboxylate (544 mg, 55.4% yield) as a red solid. ¹H NMR (400 MHz, DMSO-d₆) δ=10.10 (s, 1H), 8.59 (d, J=8.6 Hz, 1H), 8.18-8.12 (m, 2H), 7.72 (d, J=8.4 Hz, 2H), 7.57 (d, J=8.8 Hz, 1H), 7.41-7.34 (m, 4H), 4.27 (br t, J=7.6 Hz, 2H), 3.89-3.78 (m, 3H), 1.41 (s, 9H).

Step 3. To a solution of tert-butyl 3-[4-[[6-(4-fluorophenyl)-3-nitro-2-pyridyl]amino]phenyl]azetidine-1-carboxylate (464 mg, 0.999 mmol) in DMSO (4.00 mL) and methanol (1.00 mL), 2-aminopyridine-3-carbaldehyde (146 mg, 1.20 mmol) and Na₂S₂O₄ (435 mg, 2.50 mmol) were added at 25° C., and the mixture was stirred at 100° C. for 16 hrs. EtOAc (50 mL) was added and the mixture was filtered, and the filtrate was concentrated under reduce pressure to provide the crude material. Water (100 mL) was added, and the mixture was extracted with EtOAc (100 mL×3). The organic phase was washed with brine (3×100 mL), dried over Na₂SO₄, filtered and concentrated under reduced pressure to give a crude. The crude was purified by flash chromatography eluting with PE/EtOAc (from 0% to 100% of EtOAc) to give tert-butyl 3-[4-[2-(2-amino-3-pyridyl)-5-(4-fluorophenyl)imidazo[4,5-b]pyridin-3-yl]phenyl]azetidine-1-carboxylate (125 mg, 23.3% yield) as a yellow solid. ¹H NMR (400 MHz, DMSO-d₆) δ=8.27 (d, J=8.6 Hz, 1H), 8.08 (dd, J=5.8, 8.8 Hz, 2H), 8.01-7.96 (m, 2H), 7.54-7.46 (m, 4H), 7.30 (t, J=8.8 Hz, 2H), 7.20 (dd, J=1.6, 7.6 Hz, 1H), 6.96 (s, 2H), 6.42 (dd, J=4.8, 7.8 Hz, 1H), 4.30 (s, 2H), 3.96-3.86 (m, 3H), 1.41 (s, 9H).

Step 4. To a solution of tert-butyl 3-[4-[2-(2-amino-3-pyridyl)-5-(4-fluorophenyl)imidazo[4,5-b]pyridin-3-yl]phenyl]azetidine-1-carboxylate (125 mg, 0.233 mmol) in DCM (8.00 mL), TFA (2.00 mL, 0.233 mmol) was added at 25° C., and the mixture was stirred for 5 hrs. The mixture was concentrated under reduced pressure to give 3-[3-[4-(azetidin-3-yl)phenyl]-5-(4-fluorophenyl)imidazo[4,5-b]pyridin-2-yl]pyridin-2-amine (273 mg, crude) as a yellow oil, which was used directly in the next step without further purification. ¹H NMR (400 MHz, DMSO-d₆) δ=8.36 (d, J=8.6 Hz, 1H), 8.13-8.04 (m, 4H), 7.86 (dd, J=1.6, 7.6 Hz, 1H), 7.62 (s, 3H), 7.31 (t, J=8.8 Hz, 3H), 6.86 (dd, J=6.2, 7.6 Hz, 1H), 4.37-4.12 (m, 5H).

Step 5. To a solution of 3-[3-[4-(azetidin-3-yl)phenyl]-5-(4-fluorophenyl)imidazo[4,5-b]pyridin-2-yl]pyridin-2-amino (100 mg, 0.229 mmol) in DMSO (3.00 mL), DIEA (0.130 mL, 0.687 mmol) was added at 25° C. under N₂, and the mixture was stirred at 80° C. for 16 hrs. Water (20 mL) was added, and the mixture was extracted with EtOAc (20 mL×3). The organic phase was washed with brine (20 mL×3), dried over Na₂SO₄, filtered, and concentrated under reduced pressure to provide the crude material. The crude was purified by flash chromatography eluting with EtOAc/MeOH (from 0% to 60% of MeOH) to give trans-methyl 4-[[3-[4-[2-(2-amino-3-pyridyl)-5-(4-fluorophenyl)imidazo[4,5-b]pyridin-3-yl]phenyl]azetidin-1-yl]methyl]cyclohexanecarboxylate (54 mg, 0.0914 mmol, 39.90% yield) as a yellow oil. ¹H NMR (400 MHz, CDCl₃) δ=8.09 (d, J=8.6 Hz, 1H), 8.03 (dd, J=1.6, 4.8 Hz, 1H), 7.97 (dd, J=5.6, 8.8 Hz, 2H), 7.71 (d, J=8.4 Hz, 1H), 7.44-7.34 (m, 4H), 7.11-7.06 (m, 3H), 6.69 (s, 2H), 6.37 (dd, J=4.8, 7.8 Hz, 1H), 3.84 (d, J=2.0 Hz, 2H), 3.64 (s, 3H), 3.22 (s, 2H), 2.40 (d, J=7.0 Hz, 2H), 2.27-2.19 (m, 1H), 2.06-1.96 (m, 3H), 1.88 (dd, J=2.6, 13.4 Hz, 2H), 1.47-1.36 (m, 3H), 1.01-0.90 (m, 2H).

Step 6. To a solution of trans-methyl 4-[[3-[4-[2-(2-amino-3-pyridyl)-5-(4-fluorophenyl)imidazo[4,5-b]pyridin-3-yl]phenyl]azetidin-1-yl]methyl]cyclohexanecarboxylate (54.0 mg, 0.0914 mmol) in THF (1.00 mL) and water (1.00 mL), LiOH·H₂O (9.60 mg, 0.229 mmol) was added at 25° C., and the reaction mixture was stirred for 16 hrs. The mixture was concentrated under reduced pressure to provide the crude material. The crude was purified by prep-HPLC (Xtimate C18 150*40 mm*5 μm water(FA)-ACN) to give trans-4-[[3-[4-[2-(2-amino-3-pyridyl)-5-(4-fluorophenyl)imidazo[4,5-b]pyridin-3-yl]phenyl]azetidin-1-yl]methyl]cyclohexanecarboxylic acid 1-66 (23.0 mg, 42.9% yield) as a yellow solid. ¹H NMR (400 MHz, DMSO-d₆) δ=8.26 (d, J=8.6 Hz, 1H), 8.08 (dd, J=5.4, 8.8 Hz, 2H), 8.01-7.96 (m, 2H), 7.53-7.49 (m, 2H), 7.45-7.41 (m, 2H), 7.30 (t, J=8.8 Hz, 2H), 7.19 (dd, J=1.8, 7.6 Hz, 1H), 6.96 (s, 2H), 6.42 (dd, J=4.8, 7.8 Hz, 1H), 3.67-3.60 (m, 3H), 3.11 (t, J=6.0 Hz, 2H), 2.29 (d, J=6.8 Hz, 2H), 2.15-2.06 (m, 1H), 1.91-1.77 (m, 4H), 1.33-1.22 (m, 3H), 0.96-0.84 (m, 2H) HPLC Rₜ=2.485 min in 8.0 min chromatography, 10-80AB_8 min.1cm, purity 100.000%. LCMS Rₜ=2.106 min in 4.0 min chromatography, purity 100.000%, MS ESI calcd. for 576.26, [M+H]⁺ 577.26, found 577.1.

### Example 58. Synthesis of (1r,4r)-4-((3-(4-(2-(2-aminopyridin-3-yl)-6-(4-fluorophenyl)-3H-imidazo[4,5-b]pyridin-3-yl)phenyl)azetidin-1-yl)methyl)cyclohexane-1-carboxylic acid (Compound I-67)

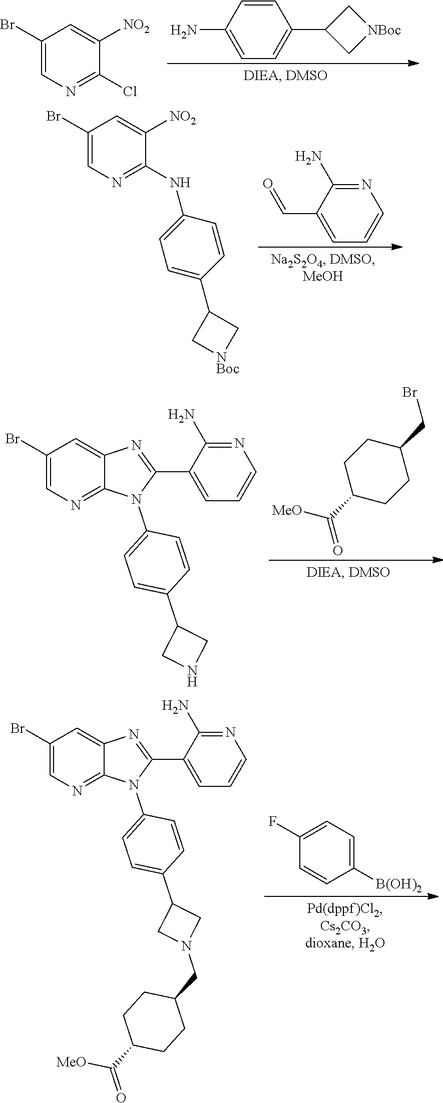

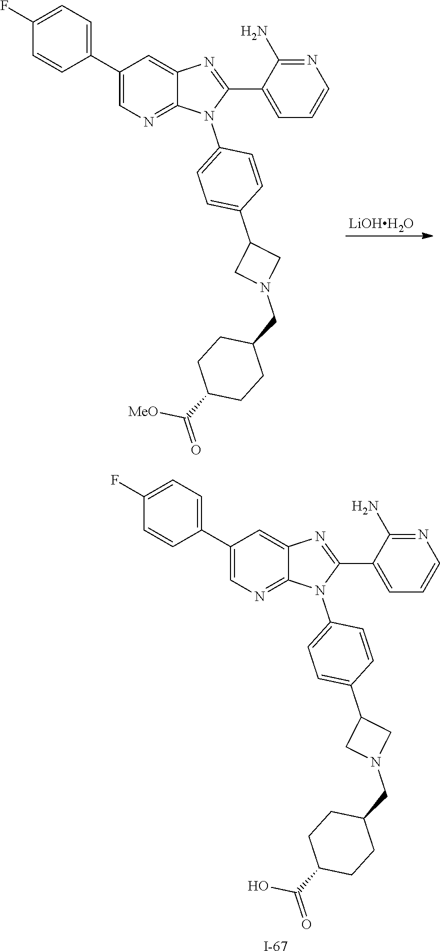

Step 1. To a solution of 5-bromo-2-chloro-3-nitro-pyridine (1.91 g, 8.05 mmol) in DMSO (20.0 mL), tert-butyl 3-(4-aminophenyl)azetidine-1-carboxylate (2.00 g, 8.05 mmol) and DIEA (4.20 mL, 24.2 mmol) were added and the mixture stirred at 100° C. for 16 hrs. Water (200 mL) was added to the reaction mixture and the whole extracted with EtOAc (100 mL 3). The organic layer was dried over Na₂SO₄ and concentrated in vacuo to provide the crude material. The crude was purified by flash column (0-50% of EtOAc in PE) to give tert-butyl 3-[4-[(5-bromo-3-nitro-2-pyridyl)amino]phenyl]azetidine-1-carboxylate (3.07 g, 84.8% yield) as a red solid. ¹H NMR (400 MHz, DMSO-d₆) δ=9.94 (s, 1H), 8.67 (d, J=2.4 Hz, 1H), 8.59 (d, J=2.4 Hz, 1H), 7.56 (d, J=8.4 Hz, 2H), 7.33 (d, J=8.4 Hz, 2H), 4.25 (t, J=7.6 Hz, 2H), 3.87-3.75 (m, 3H), 1.41 (s, 9H).

Step 2. To a solution of tert-butyl 3-[4-[(5-bromo-3-nitro-2-pyridyl)amino]phenyl]azetidine-1-carboxylate (2.70 g, 6.01 mmol) in DMSO (30 mL) and methanol (10 mL), 2-aminopyridine-3-carbaldehyde (1.03 g, 8.41 mmol) and Na₂S₂O₄ (4.18 g, 24.0 mmol) were added and the reaction was stirred at 100° C. for 16 hrs water (300 mL) was added to the reaction mixture and the whole was extracted with EtOAc (200 mL×3). The organic layer was dried over Na₂SO₄ and concentrated in vacuo to provide the crude material. The aqueous layer was purified by prep-HPLC (column: Welch Xtimate C18 150*40 mm*5 um; mobile phase: water (FA)-ACN; B %:1%-21%, 60 min) to give 3-[3-[4-(azetidin-3-yl)phenyl]-6-bromo-imidazo[4,5-b]pyridin-2-yl]pyridin-2-amine (600 mg, 23.7% yield) as a white solid. ¹H NMR (400 MHz, DMSO-d₆) δ=8.55-8.48 (m, 1H), 8.41 (d, J=2.0 Hz, 1H), 8.00 (dd, J=1.6, 4.8 Hz, 1H), 7.55-7.42 (m, 2H), 7.41-7.34 (m, 2H), 7.23 (d, J=8.0 Hz, 1H), 7.01-6.87 (m, 2H), 6.46-6.37 (m, 1H), 3.98-3.70 (m, 3H), 3.63-3.56 (m, 2H).

Step 3. To a solution of 3-[3-[4-(azetidin-3-yl)phenyl]-6-bromo-imidazo[4,5-b]pyridin-2-yl]pyridin-2-amine (300 mg, 0.712 mmol) in DMSO (3.00 mt), trans-methyl 4-(bromomethyl)cyclohexanecarboxylate (234 mg, 0.997 mmol) and DIEA were added and the reaction stirred at 80° C. for 16 hrs. Water (30 mL) was added to the reaction mixture and the whole extracted with EtOAc (20 mL×3). The organic layer was dried over Na₂SO₄ and concentrated in vacuo to provide the crude material. The crude was purified by flash column (0-30% of MeOH in DCM) to give trans-methyl 4-[[3-[4-[2-(2-amino-3-pyridyl)-6-bromo-imidazo[4,5-b]pyridin-3-yl]phenyl]azetidin-1-yl]methyl]cyclohexanecarboxylate (100 mg, 24.4% yield) as a yellow solid. ¹H NMR (400 MHz, DMSO-d₆) δ=8.50 (d, J=2.0 Hz, 1H), 8.41 (d, J=2.0 Hz, 1H), 8.00 (dd, J=2.0, 4.8 Hz, 1H), 7.48 (d, J=8.4 Hz, 2H), 7.37 (d, J=8.4 Hz, 2H), 7.24 (dd, J=2.0, 7.6 Hz, 1H), 6.92 (s, 2H), 6.42 (dd, J=4.8, 7.6 Hz, 1H), 3.57 (s, 6H), 3.10 (s, 1H), 2.54 (s, 1H), 2.34-2.24 (m, 2H), 1.92-1.70 (m, 4H), 1.36-1.20 (m, 4H), 1.01-0.82 (m, 2H).

Step 4. To a solution of trans-methyl 4-[[3-[4-[2-(2-amino-3-pyridyl)-6-bromo-imidazo[4,5-b]pyridin-3-yl]phenyl]azetidin-1-yl]methyl]cyclohexanecarboxylate (100 mg, 0.174 mmol) in 1,4-dioxane (3.00 mL) and water (1.00 mL), (4-fluorophenyl)boronic acid (29.0 mg, 0.209 mmol), Pd(dppf)Cl₂ (13.0 mg, 0.0174 mmol) and Cs₂CO₃ (170 mg, 0.521 mmol) were added at 25° C., and the mixture was stirred at 90° C. for 14 hrs under N₂. Water (30 mL) was added to the reaction mixture and the whole extracted with EtOAc (20 mL×3). The organic layer was dried over Na₂SO₄ and concentrated in vacuo to provide the crude material. The crude was purified by flash column (0-30% of MeOH in DCM) to give trans-methyl 4-[[3-[4-[2-(2-amino-3-pyridyl)-6-(4-fluorophenyl)imidazo[4,5-b]pyridin-3-yl]phenyl]azetidin-1-yl]methyl]cyclohexanecarboxylate (100 mg, 97.4% yield) as a yellow solid. ¹H NMR (400 MHz, DMSO-d₆) δ=8.60 (d, J=2.0 Hz, 1H), 8.46 (d, J=2.0 Hz, 1H), 8.00 (dd, J=1.6, 4.8 Hz, 1H), 7.86-7.80 (m, 2H), 7.53-7.49 (m, 3H), 7.41-7.35 (m, 3H), 7.24 (dd, J=1.6, 7.6 Hz, 1H), 7.02 (s, 2H), 6.42 (dd, J=4.8, 7.6 Hz, 1H), 3.58 (s, 3H), 3.17 (d, J=5.2 Hz, 5H), 2.28-2.18 (m, 2H), 1.93-1.73 (m, 5H), 1.37-1.17 (m, 5H). ¹⁹F NMR (376.5 MHz, DMSO-d₆) δ=−115.350.

Step 5. To a solution of trans-methyl 4-[[3-[4-[2-(2-amino-3-pyridyl)-6-(4-fluorophenyl)imidazo[4,5-b]pyridin-3-yl]phenyl]azetidin-1-yl]methyl]cyclohexanecarboxylate (100 mg, 0.169 mmol) in THF (1 mL) and water (1 mL), LiOH·H₂O (28 mg, 0.677 mmol) was added and the reaction then stirred at 25° C. for 16 hrs. The reaction mixture was purified by prep-HPLC (column: Welch Xtimate C18 150*40 mm*5 μm; mobile phase: water(FA)-ACN; B %: 10%-40%, 60 min) to give trans-4-[[3-[4-[2-(2-amino-3-pyridyl)-6-(4-fluorophenyl)imidazo[4,5-b]pyridin-3-yl]phenyl]azetidin-1-yl]methyl]cyclohexanecarboxylic acid 1-67 (35 mg, 34.9% yield) as a yellow solid. LCMS Rₜ=2.802 min in 4 min chromatography, purity 99.5%, MS ESI calcd. For 576.26 [M+H]⁺ 577.26, found 577.1. HPLC Rₜ=4.117 min in 8 min chromatography, purity 98.4%. ¹H NMR (400 MHz, DMSO-d₆) δ=8.60 (d, J=2.4 Hz, 1H), 8.46 (d, J=2.0 Hz, 1H), 8.00 (dd, J=1.6, 4.8 Hz, 1H), 7.87-7.77 (m, 2H), 7.54-7.47 (m, 2H), 7.44-7.31 (m, 4H), 7.24 (dd, J=1.6, 7.6 Hz, 1H), 7.02 (s, 2H), 6.42 (dd, J=4.8, 7.6 Hz, 1H), 3.72-3.60 (m, 3H), 3.15-3.11 (m, 2H), 2.31 (d, J=6.8 Hz, 2H), 2.15-2.04 (m, 1H), 1.92-1.76 (m, 4H), 1.34-1.19 (m, 3H), 1.00-0.80 (m, 2H). ¹⁹F NMR (376.5 MHz, DMSO-d₆) δ=−115.350.

### Example 59. Synthesis of (1r,4r)-4-((3-(4-(2-(2-aminopyridin-3-yl)-6-(pyridin-2-yl)-1H-benzo[d]imidazol-1-yl)phenyl)azetidin-1-yl)methyl)cyclohexane-1-carboxylic acid (Compound I-68)

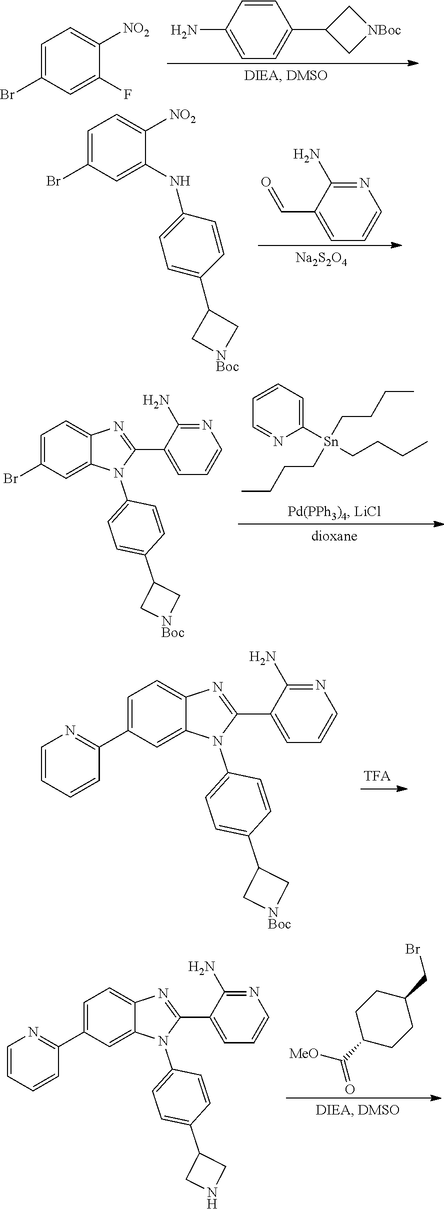

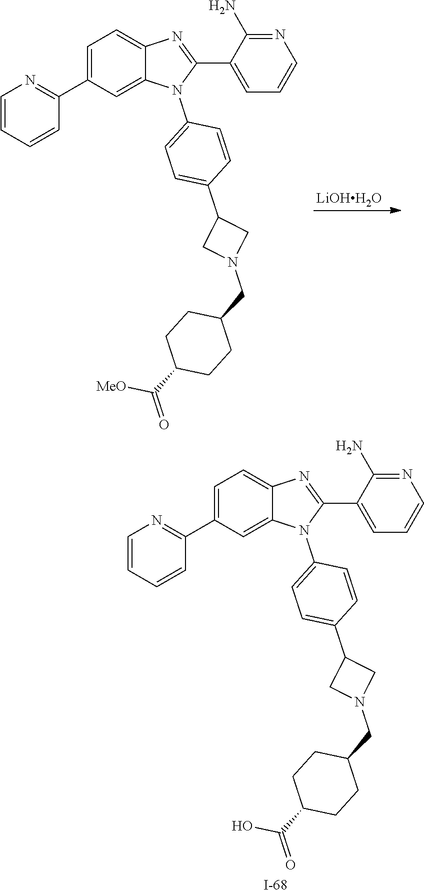

Step 1. To a stirred solution of 4-bromo-2-fluoro-1-nitro-benzene (2.00 g, 9.10 mmol) and tert-butyl 3-(4-aminophenyl)azetidine-1-carboxylate (3.00 g, 12.1 mmol) in DMSO (30.0 mL), DIEA (5.00 mL, 27.3 mmol) was added and the reaction mixture was stirred at 95° C. under N₂ for 12 hrs. Water (300 mL) was added to the reaction mixture and the whole extracted with EtOAc (150 mL×3). The organic layer was dried over Na₂SO₄ and concentrated in vacuo to provide the crude material. The crude was purified by flash column (0-50% of EtOAc in PE) to give tert-butyl 3-[4-(5-bromo-2-nitro-anilino)phenyl]azetidine-1-carboxylate (4.00 g, 98.1% yield) as a red solid. ¹H NMR (400 MHz, DMSO-d₆) δ=9.48 (s, 1H), 8.05 (d, J=9.2 Hz, 1H), 7.46-7.38 (m, 2H), 7.37-7.29 (m, 2H), 7.17 (d, J=2.0 Hz, 1H), 7.01 (dd, J=2.0, 9.2 Hz, 1H), 4.35-4.20 (m, 2H), 3.92-3.77 (m, 3H), 1.41 (s, 9H).

Step 2. To a stirred solution of 2-aminopyridine-3-carbaldehyde (2.10 g, 17.8 mmol) and tert-butyl 3-[4-(5-bromo-2-nitro-anilino)phenyl]azetidine-1-carboxylate (4.00 g, 8.90 mmol) in DMSO (70.0 mL) and methanol (8.00 mL), Na₂S₂O₄ (6.20 g, 35.7 mmol) was added and the reaction mixture was stirred at 95° C. under O₂ for 12 hrs. The reaction mixture was diluted with EtOAc (200 mL), washed with water (200 mL) and brine (200 mL×2), dried over anhydrous sodium sulfate, filtered and the filtrate was concentrated to afford a residue. The crude product was purified by flash chromatography on silica gel (95% of EtOAc in PE) to afford tert-butyl 3-[4-[2-(2-amino-3-pyridyl)-6-bromo-benzimidazol-1-yl]phenyl]azetidine-1-carboxylate (2.00 g, 48.0% yield) as a yellow solid. ¹H NMR (400 MHz, DMSO-d₆) δ=7.97 (dd, J=2.0, 4.8 Hz, 1H), 7.76 (d, J=8.4 Hz, 1H), 7.61-7.39 (m, 5H), 7.30 (d, J=2.0 Hz, 1H), 7.15 (dd, J=1.6, 7.6 Hz, 1H), 6.97 (s, 2H), 6.39 (dd, J=4.8, 7.6 Hz, 1H), 4.36-4.23 (m, 2H), 3.95-3.85 (m, 3H), 1.41 (s, 9H).

Step 3. To a stirred solution of tert-butyl 3-[4-[2-(2-amino-3-pyridyl)-6-bromo-benzimidazol-1-yl]phenyl]azetidine-1-carboxylate (700 mg, 1.35 mmol) and tributyl(2-pyridyl)stannane (0.860 mL, 2.69 mmol) in 1,4-dioxane (15.0 mL), LiCl (171 mg, 4.04 mmol) and Pd(PPh₃)₄ (155 mg, 0.140 mmol) were added under N₂, and the reaction mixture was stirred at 100° C. under N₂ for 14 hrs. The reaction mixture was filtered, and the filtrate was diluted with EtOAc (100 mL), washed with water (100 mL) and brine (100 mL×2). The combined organic layer was dried over anhydrous sodium sulfate, filtered and the filtrate was concentrated to afford a residue. The crude product was purified by flash chromatography on silica gel (95% EtOAc in PE) to afford tert-butyl 3-[4-[2-(2-amino-3-pyridyl)-6-(2-pyridyl) benzimidazol-1-yl]phenyl]azetidine-1-carboxylate (600 mg, 86.0% yield) as a yellow oil. ¹H NMR (400 MHz, CDCl₃) δ=8.72-8.61 (m, 1H), 8.06 (dd, J=1.6, 4.8 Hz, 1H), 7.99-7.87 (m, 3H), 7.80-7.73 (m, 2H), 7.53-7.47 (m, 2H), 7.37 (d, J=8.4 Hz, 2H), 7.22 (dt, J=3.2, 5.2 Hz, 1H), 7.06 (dd, J=1.6, 7.6 Hz, 1H), 6.62 (s, 2H), 6.37 (dd, J=5.2, 8.0 Hz, 1H), 4.42 (t, J=8.8 Hz, 2H), 4.06 (dd, J=6.0, 8.4 Hz, 2H), 3.91-3.76 (m, 1H), 1.50 (s, 9H).

Step 4. To a stirred solution of tert-butyl 3-[4-[2-(2-amino-3-pyridyl)-6-(2-pyridyl)benzimidazol-1-yl]phenyl]azetidine-1-carboxylate (300 mg, 0.580 mmol) in DCM (2.00 mL), TFA (1.00 mL, 0.580 mmol) was added and the reaction mixture was stirred at 20° C. under N₂ for 12 hrs. The reaction mixture was quenched with saturated aqueous NaHCO₃ solution (15 mL) and extracted with methanol in DCM 10% solution (10 mL×3). The combined organic layers were dried over anhydrous sodium sulfate, filtered and the filtrate was concentrated to afford 3-[1-[4-(azetidin-3-yl)phenyl]-6-(2-pyridyl)benzimidazol-2-yl]pyridin-2-amine (80.0 mg, 33.1% yield) as a yellow oil. ¹H NMR (400 MHz, DMSO-d₆) δ=8.63-8.55 (m, 1H), 8.06 (dd, J=1.6, 8.4 Hz, 1H), 8.01-7.96 (m, 2H), 7.95-7.83 (m, 3H), 7.68-7.59 (m, 3H), 7.56-7.52 (m, 2H), 7.37-7.29 (m, 1H), 7.18 (dd, J=2.0, 7.6 Hz, 1H), 7.03 (s, 2H), 6.39 (dd, J=4.8, 7.6 Hz, 1H), 4.34-4.09 (m, 5H).

Step 5. To a stirred solution of 3-[1-[4-(azetidin-3-yl)phenyl]-6-(2-pyridyl)benzimidazol-2-yl]pyridin-2-amine (100 mg, 0.240 mmol) and trans-methyl 4-(bromomethyl)cyclohexanecarboxylate (84.0 mg, 0.360 mmol) in DMSO (3.00 mL), DIEA (0.130 mL, 0.720 mmol) was added and the reaction mixture was stirred at 80° C. under N₂ for 15 hrs. The reaction mixture was diluted with EtOAc (20 mL), washed with water (50 mL) and brine (50 mL×2), dried over anhydrous sodium sulfate, filtered and the filtrate was concentrated to afford a residue. The crude product was purified by flash chromatography on silica gel (15% of methanol in DCM) to afford trans-methyl 4-[[3-[4-[2-(2-amino-3-pyridyl)-6-(2-pyridyl)benzimidazol-1-yl]phenyl]azetidin-1-yl]methyl]cyclohexanecarboxylate (40 mg, 29.2% yield) as a yellow oil. LCMS Rₜ=0.750 min in 1.5 min chromatography, purity 73.778%, MS ESI calcd. for 572.29 [M+H]⁺ 573.29, found 573.3.

Step 6. To a stirred solution of trans-methyl 4-[[3-[4-[2-(2-amino-3-pyridyl)-6-(2-pyridyl)benzimidazol-1-yl]phenyl]azetidin-1-yl]methyl]cyclohexanecarboxylate (40.0 mg, 0.0700 mmol) in THF (1.00 mL) and water (1.00 mL), LiOH·H₂O (8.40 mg, 0.210 mmol) was added and the reaction mixture was stirred at 20° C. under N₂ for 15 hrs. The reaction mixture was concentrated and the pH adjusted to pH ~5 with 1N HCl. The crude product was purified by prep-HPLC (column: Welch Xtimate C18 150×30 mm×5 nm, method: water (FA)-ACN, begin B: 10, end B 40) to afford trans-4-[[3-[4-[2-(2-amino-3-pyridyl)-6-(2-pyridyl)benzimidazol-1-yl]phenyl]azetidin-1-yl]methyl]cyclohexanecarboxylic acid I-68 (13.0 mg, 32.9% yield) as a white solid. ¹H NMR (400 MHz, DMSO-d₆) δ=12.12 (s, 1H), 8.61 (d, J=4.0 Hz, 1H), 8.23-7.78 (m, 7H), 7.58 (d, J=8.4 Hz, 2H), 7.44 (d, J=8.4 Hz, 2H), 7.31 (dd, J=5.2, 6.8 Hz, 1H), 7.15 (dd, J=1.6, 7.6 Hz, 1H), 7.07 (s, 2H), 6.39 (dd, J=4.8, 7.6 Hz, 1H), 3.80-3.65 (m, 3H), 3.20 (s, 2H), 2.35 (br d, J=6.0 Hz, 2H), 2.18-2.03 (m, 1H), 1.96-1.72 (m, 4H), 1.37-1.13 (m, 3H), 1.02-0.82 (m, 2H). HPLC Rₜ=2.408 min in 8 min chromatography, purity 99.474%. LCMS Rₜ=1.515 min in 4 min chromatography, purity 98.979%, MS ESI calcd. for 558.27 [M+H]⁺ 559.27, found 559.2.

### Example 60, 61 and 62. Synthesis of (1R,4S)-4-((3-(4-(2-(2-aminopyridin-3-yl)-5-phenyl-3H-imidazo[4,5-b]pyridin-3-yl)phenyl)azetidin-1-yl)methyl)-2,2-dimethylcyclohexane-1-carboxylic acid (Compound I-69), (1S,4R)-4-((3-(4-(2-(2-aminopyridin-3-yl)-5-phenyl-3H-imidazo[4,5-b]pyridin-3-yl)phenyl)azetidin-1-yl)methyl)-2,2-dimethylcyclohexane-1-carboxylic acid (Compound I-70) and (1R,4R)-4-((3-(4-(2-(2-aminopyridin-3-yl)-5-phenyl-3H-imidazo[4,5-b]pyridin-3-yl)phenyl)azetidin-1-yl)methyl)-2,2-dimethylcyclohexane-1-carboxylic acid (Compound I-71)

### Synthesis of methyl 4-formyl-2,2-dimethylcyclohexane-1-carboxylate

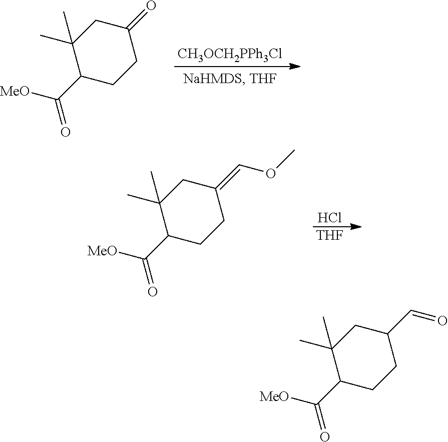

Step 1. To a solution of CH₃OCH₂PPh₃Cl (4.18 g, 12.2 mmol) in THF (15.0 mL), NaHMDS (12.0 mL, 12.2 mmol) was added slowly at −40° C. for 10 mins under N₂ atmosphere. After addition, the mixture was stirred at −40° C. for 1 hr and subsequently at 0° C. for 30 mins. After cooling to −40° C., a solution of methyl 2,2-dimethyl-4-oxocyclohexane-1-carboxylate (1.50 g, 8.14 mmol) in THF (10.0 mL) was added dropwise at −40° C. The resulting mixture was stirred at 0° C. for 1 hr under N₂ atmosphere. The reaction was quenched with aqueous saturated NH₄Cl and extracted with EtOAc (100 mL×3) and dried over anhydrous Na₂SO₄, filtered and concentrated. The residue was purified by Combi-Flash column (EtOAc in PE=0%-3%) to give methyl (E)-4-(methoxymethylene)-2,2-dimethylcyclohexane-1-carboxylate (1.70 g, 98.36%) as a colorless oil. ¹H NMR (400 MHz, DMSO-d₆) δ=5.99-5.74 (m, 1H), 3.57 (s, 3H), 3.46 (d, J=11.6 Hz, 3H), 2.62-2.52 (m, 1H), 2.31-2.24 (m, 1H), 2.11-2.02 (m, 1H), 1.87-1.75 (m, 1H), 1.65-1.54 (m, 2H), 1.21-1.14 (m, 1H), 0.94 (d, J=8.4 Hz, 3H), 0.80 (d, J=7.6 Hz, 3H).

Step 2. To a solution of methyl (E)-4-(methoxymethylene)-2,2-dimethylcyclohexane-1-carboxylate (500 mg, 2.36 mmol) in THF (3.00 mL), HCl (3.10 mL, 9.42 mmol, 3M) was added at 25° C., and the mixture was stirred for 3 hrs. Saturated aqueous NaHCO₃ (10 mL) was added to the mixture, the whole was extracted with ethyl acetate (25 mL×3), the organic phases was dried over anhydrous sodium sulfate, and concentrated to give methyl 4-formyl-2,2-dimethylcyclohexane-1-carboxylate (400 mg, 85.7% yield) as a colorless oil. ¹H NMR (400 MHz, DMSO-d₆) δ=9.63-9.49 (m, 1H), 3.63-3.50 (m, 3H), 2.31-2.11 (m, 1H), 1.82-1.53 (m, 4H), 1.47 (dd, J=4.8, 13.2 Hz, 1H), 1.23-1.10 (m, 1H), 1.02-0.83 (m, 6H).

### Synthesis of (1R,4S)-4-((3-(4-(2-(2-aminopyridin-3-yl)-5-phenyl-3H-imidazo[4,5-b]pyridin-3-yl)phenyl)azetidin-1-yl)methyl)-2,2-dimethylcyclohexane-1-carboxylic acid (Compound I-69), (1S,4R)-4-((3-(4-(2-(2-aminopyridin-3-yl)-5-phenyl-3H-imidazo[4,5-b]pyridin-3-yl)phenyl)azetidin-1-yl)methyl)-2,2-dimethylcyclohexane-1-carboxylic acid (Compound I-70) and (1R,4R)-4-((3-(4-(2-(2-aminopyridin-3-yl)-5-phenyl-31H-imidazo[4,5-b]pyridin-3-yl)phenyl)azetidin-1-yl)methyl)-2,2-dimethylcyclohexane-1-carboxylic acid (Compound I-71)

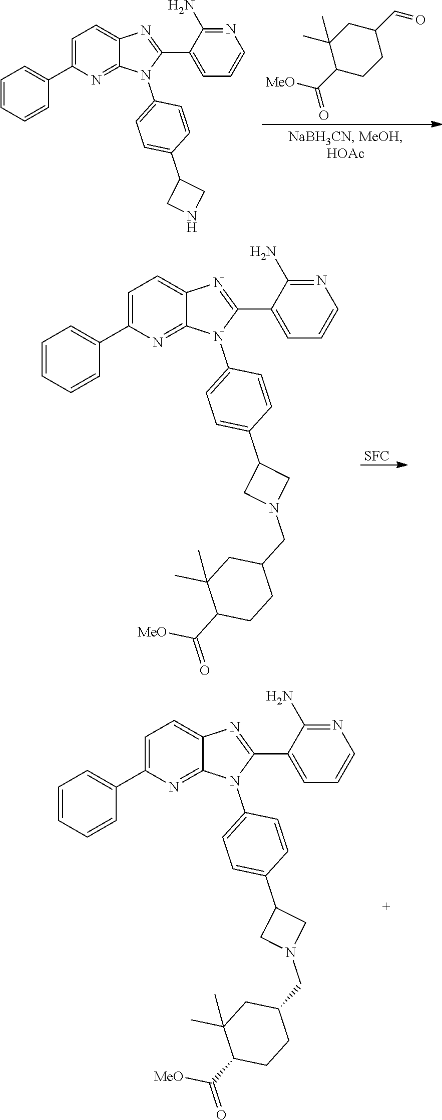 [SMILES: Nc1ncccc1-c1nc2ccc(-c3ccccc3)nc2n1-c1ccc(C2CNC2)cc1 ; COC(=O)C1CCC(C=O)CC1(C)C ; COC(=O)C1CCC(CN2CC(c3ccc(-n4c(-c5cccnc5N)nc5ccc(-c6ccccc6)nc54)cc3)C2)CC1(C)C ; "SFC" ; COC(=O)[C@H]1CC[C@@H](CN2CC(c3ccc(-n4c(-c5cccnc5N)nc5ccc(-c6ccccc6)nc54)cc3)C2)CC1(C)C]

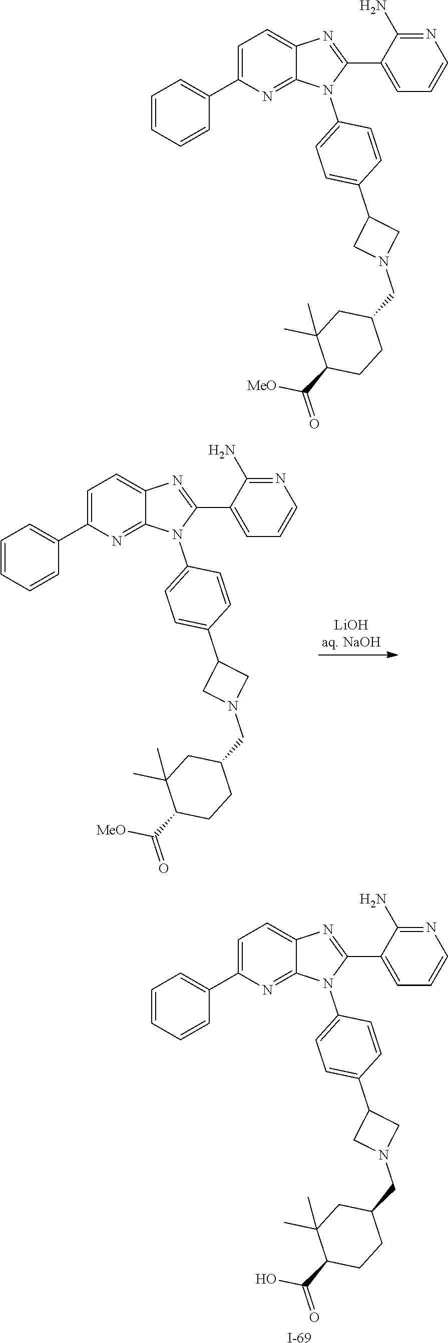

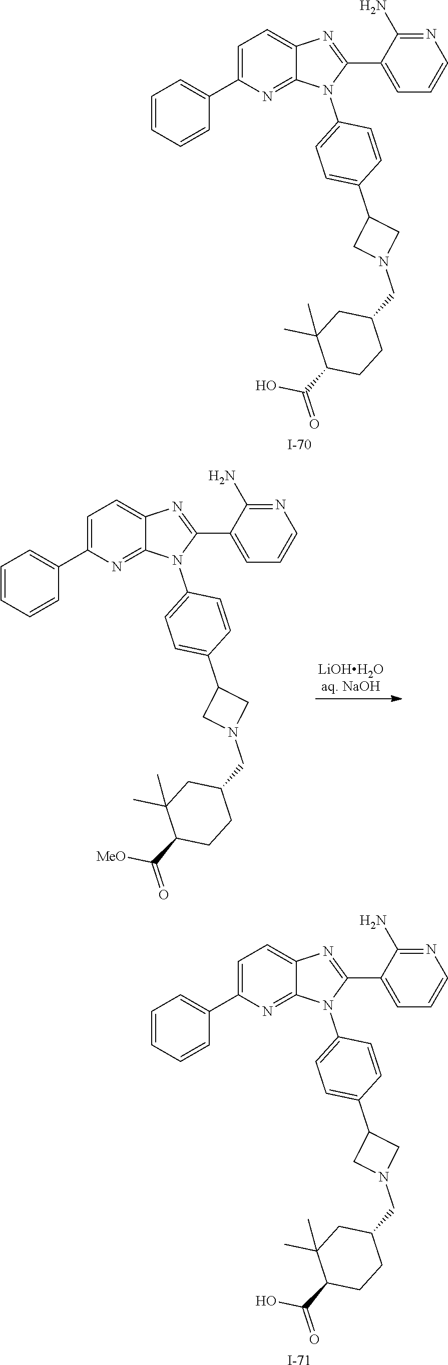

Step 1. To a solution of methyl 4-formyl-2,2-dimethylcyclohexane-1-carboxylate (387 mg, 1.95 mmol) in methanol (3.00 mL), 3-(3-(4-(azetidin-3-yl)phenyl)-5-phenyl-3H-imidazo[4,5-b]pyridin-2-yl)pyridin-2-amine (510 mg, 1.22 mmol) and TEA (370 mg, 3.66 mmol) were added and the mixture was stirred at 25° C. for 0.5 hr. NaBH₃CN (230 mg, 3.66 mmol) and AcOH (2 drops) were added to the mixture and the reaction mixture was stirred at 25° C. for 1 hr. Water (20 mL) was added to the mixture and the mixture was extracted with EtOAc (20 mL×3). The organic phase was washed with brine (20 mL), dried over Na₂SO₄, filtered and concentrated. The residue was purified by Combi-Flash column (EtOAc in PE=0%-100%) to give methyl 4-((3-(4-(2-(2-aminopyridin-3-yl)-5-phenyl-3H-imidazo[4,5-b]pyridin-3-yl)phenyl)azetidin-1-yl)methyl)-2,2-dimethylcyclohexane-1-carboxylate (450 mg, 61.5% yield) as a yellow oil.

Step 2. Methyl 4-[[3-[4-[2-(2-amino-3-pyridyl)-5-phenyl-imidazo[4,5-b]pyridin-3-yl]phenyl]azetidin-1-yl]methyl]-2,2-dimethyl-cyclohexanecarboxylate (600 mg, 0.999 mmol) was purified by prep-HPLC (DAICEL CHIRALPAK AD(250 mm*30 mm,10 μm); mobile phase: [C02-i-PrOH(0.1% NH₃H₂O)]; B %: 55%-55%) to give cis-methyl (1S,4S)-4-[[3-[4-[2-(2-amino-3-pyridyl)-5-phenyl-imidazo[4,5-b]pyridin-3-yl]phenyl]azetidin-1-yl]methyl]-2,2-dimethyl-cyclohexanecarboxylate (370 mg, 61.7% yield) as a white solid and trans-methyl (1R,4S)-4-[[3-[4-[2-(2-amino-3-pyridyl)-5-phenyl-imidazo[4,5-b]pyridin-3-yl]phenyl]azetidin-1-yl]methyl]-2,2-dimethyl-cyclohexanecarboxylate (150 mg, 25.0% yield) as a white solid. ¹H NMR (400 MHz, DMSO-d₆) δ=8.17 (d, J=8.3 Hz, 1H), 7.97-7.84 (m, 4H), 7.48-7.26 (m, 7H), 7.12 (br d, J=7.2 Hz, 1H), 6.83 (br s, 2H), 6.33 (dd, J=4.8, 7.2 Hz, 1H), 4.25 (d, J=4.0 Hz, 2H), 3.95 (s, 1H), 3.74-3.62 (m, 3H), 3.48 (s, 3H), 2.06-2.00 (m, 1H), 1.71-1.64 (m, 1H), 1.56-1.47 (m, 3H), 1.35 (br s, 1H), 1.14 (br s, 1H), 0.87-0.77 (m, 8H). ¹H NMR (400 MHz, DMSO-d₆) δ=8.27 (d, J=8.4 Hz, 1H), 8.08-7.92 (m, 4H), 7.55-7.37 (m, 7H), 7.20 (br d, J=7.2 Hz, 1H), 6.95 (s, 2H), 6.43 (dd, J=4.8, 7.6 Hz, 1H), 3.64 (br d, J=6.0 Hz, 2H), 3.60-3.54 (m, 3H), 3.13-3.06 (m, 2H), 2.27 (br d, J=6.0 Hz, 2H), 1.78-1.22 (m, 7H), 1.04 (d, J=6.0 Hz, 2H), 0.99-0.93 (m, 3H), 0.90-0.86 (m, 3H).

Step 3a. To a solution of cis-methyl (1R,4R)-4-[[3-[4-[2-(2-amino-3-pyridyl)-5-phenyl-imidazo[4,5-b]pyridin-3-yl]phenyl]azetidin-1-yl]methyl]-2,2-dimethyl-cyclohexanecarboxylate (350 mg, 0.583 mmol) in THF (1.50 mL), water (0.500 mL) and methanol (2.00 mL), LiOH (42.0 mg, 1.75 mmol) and NaOH (467 mg, 11.7 mmol) were added under N₂ atmosphere. The mixture was stirred at 80° C. for 6 hrs. The residue was purified by prep-HPLC (Column: Xtimate C18 200*40 mm*7 μm; mobile phase: [water (NH₄HCO₃)-ACN]; B % 14%-54%, 25 min) to give (1R,4S)-4-((3-(4-(2-(2-aminopyridin-3-yl)-5-phenyl-3H-imidazo[4,5-b]pyridin-3-yl)phenyl)azetidin-1-yl)methyl)-2,2-dimethylcyclohexane-1-carboxylic acid I-69 (59.0 mg, 17.3% yield) as a white solid and trans-(1S,4R)-4-[[3-[4-[2-(2-amino-3-pyridyl)-5-phenyl-imidazo[4,5-b]pyridin-3-yl]phenyl]azetidin-1-yl]methyl]-2,2-dimethyl-cyclohexanecarboxylic acid I-70 (80.0 mg, 23.3% yield) as a white solid ¹H NMR (400 MHz, DMSO-d₆) δ=8.26 (d, J=8.4 Hz, 1H), 8.08-7.92 (m, 4H), 7.53-7.34 (m, 7H), 7.19 (dd, 1=1.6, 7.6 Hz, 1H), 6.96 (s, 2H), 6.41 (dd, J=4.8, 7.6 Hz, 1H), 3.73-3.60 (m, 3H), 3.08 (t, J=6.4 Hz, 2H), 2.24 (d, J=6.4 Hz, 2H), 2.03-1.93 (m, 1H), 1.80-1.70 (m, 1H), 1.62-1.40 (m, 4H), 0.97 (s, 3H), 0.90 (s, 3H), 0.87-0.74 (m, 2H). HPLC Rₜ=2.198 min in 8 min chromatography, purity 99.84%. LCMS Rₜ=1 481 min in 4 min chromatography, purity 99.94%, MS ESI calcd. for 586.74 [M+H]⁺ 587.74 [M+H]⁺ 587.3. ¹H NMR (400 MHz, DMSO-d₆) δ=8.26 (d, J=8.4 Hz, 1H), 8.06-7.94 (m, 4H), 7.54-7.36 (m, 7H), 7.19 (dd, J=1.6, 7.6 Hz, 1H), 6.96 (s, 2H), 6.42 (dd, J=4.8, 7.6 Hz, 1H), 3.73-3.60 (m, 3H), 3.08 (t, 0.1=6.4 Hz, 2H), 2.31-2.23 (m, 2H), 2.15 (d, J=2.4 Hz, 1H), 1.78-1.60 (m, 2H), 1.50 (d, J=7.2 Hz, 3H), 1.36-122 (m, 1H), 1.21-1.14 (m, 1H), 0.94 (d, J=7.6 Hz, 6H). HPLC Rₜ=2.294 min in 8 min chromatography, purity 99.95%. LCMS Rₜ=1.550 min in 4 min chromatography, purity 99.90%, MS ESI calcd. for 586.31 [M+H]⁺ 587.31 [M+H]⁺ 587.3.

Step 3b. To a solution of cis-methyl (1R,4R)-4-[[3-[4-[2-(2-amino-3-pyridyl)-5-phenyl-imidazo[4,5-b]pyridin-3-yl]phenyl]azetidin-1-yl]methyl]-2,2-dimethyl-cyclohexanecarboxylate (150 mg, 0.250 mmol) in THF (3.00 mL), water (1.00 mL) and methanol (2.00 mL), LiOH·H₂O (18 mg, 0.749 mmol) and NaOH (86.0 mg, 2.14 mmol) were added under N₂ atmosphere and the mixture was stirred at 60° C. for 6 hrs. The residue was purified by prep-HPLC (Column: Xtimate C18 200*40 mm*7 μm; mobile phase: [water (NH₄HCO₃)-ACN]; B %: 10%-50%, 25 min) to give cis-(1R,4R)-4-[[3-[4-[2-(2-amino-3-pyridyl)-5-phenyl-imidazo[4,5-b]pyridin-3-yl]phenyl]azetidin-1-yl]methyl]-2,2-dimethyl-cyclohexanecarboxylic acid I-71 (29.0 mg, 0.0498 mmol, 19.9% yield) as a white solid. ¹H NMR (400 MHz, DMSO-d₆) δ 8.26 (d, J=8.4 Hz, 1H), 8.07-7.94 (m, 4H), 7.55-7.36 (m, 7H), 7.20 (dd, J=1.2, 7.2 Hz, 1H), 6.95 (s, 2H), 6.42 (dd, J=4.8, 7.6 Hz, 1H), 3.69 (br d, J=7.2 Hz, 1H), 3.66-3.60 (m, 2H), 3.08 (br t, J=6.4 Hz, 2H), 2.24 (br d, J=6.4 Hz, 2H), 1.99 (dd, J=4.8, 11.2 Hz, 1H), 1.76 (br d, J=11.2 Hz, 1H), 1.62-1.42 (m, 4H), 0.97 (s, 3H), 0.90 (s, 3H), 0.84 (br t, J=12.4 Hz, 2H). HPLC Rₜ=3.479 min in 8 min chromatography, purity 100%. LCMS Rₜ=1.562 min in 4 min chromatography, purity 100%, MS ESI calcd. for 586.31 [M+H]⁺ 587.31. found 587.3.

### Example 63. Synthesis of (1r,4r)-4-((3-(4-(2-(2-aminopyridin-3-yl)-6-(1H-pyrazol-1-yl)-1H-benzo[d]imidazol-1-yl)phenyl)azetidin-1-yl)methyl)cyclohexane-1-carboxylic acid (Compound I-72)

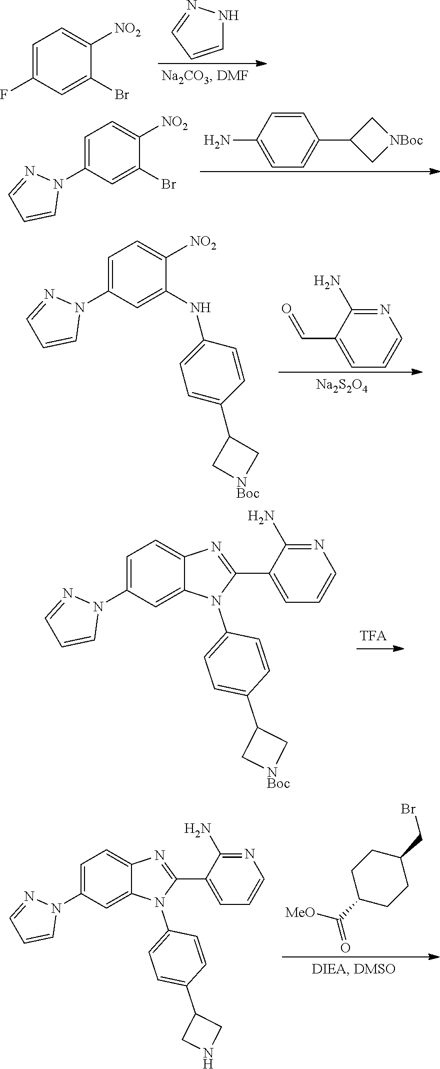

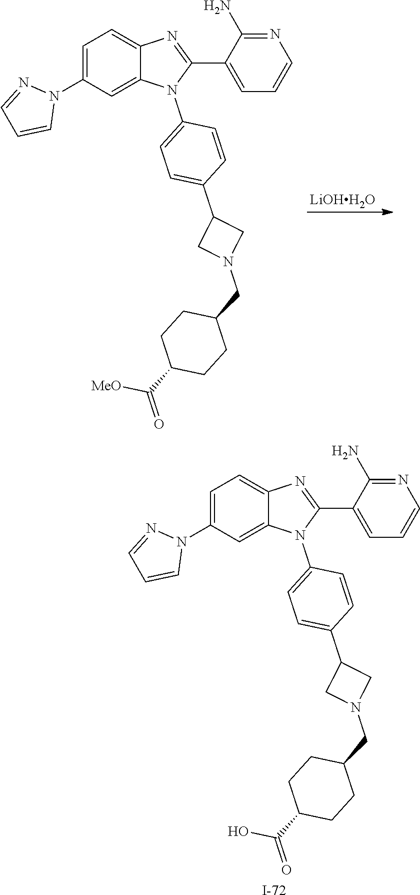

Step 1. To a stirred solution of 1H-pyrazole (681 mg, 10.0 mmol) and 2-bromo-4-fluoro-1-nitro-benzene (2.00 g, 9.10 mmol) in DMF (15.0 mL), Na₂CO₃ (2.40 g, 22.7 mmol) was added and the reaction mixture was stirred at 100° C. under N₂ for 12 hrs. The reaction mixture was diluted with EtOAc (50 mL), washed with water (50 mL) and brine (50 mL×2), dried over anhydrous sodium sulfate, filtered and the filtrate was concentrated to afford a residue. The crude product was purified by flash chromatography on silica gel (20% of EtOAc in PE) to afford 1-(3-bromo-4-nitro-phenyl)pyrazole (1.20 g, 49.2% yield). LCMS Rₜ=0.921 min in 1.5 min chromatography, purity 54.649%, MS ESI calcd. for 266.96 [M+H]⁺ 267.96, found 267.9.

Step 2. To a stirred solution of 1-(3-bromo-4-nitro-phenyl)pyrazole (1.00 g, 3.70 mmol), Cs₂CO₃ (3.70 g, 11.2 mmol), and tert-butyl 3-(4-aminophenyl)azetidine-1-carboxylate (1.40 g, 5.60 mmol) in 1,4-dioxane (20.0 mL), XantPhos-Pd-G3 (354 mg, 0.370 mmol) was added under N₂, and the reaction was stirred at 80° C. under N₂ for 12 hrs. The reaction mixture was diluted with EtOAc (200 mL), washed with water (200 mL) and brine (200 mL×2), dried over anhydrous sodium sulfate, filtered and the filtrate was concentrated to afford a residue. The crude product was purified by flash chromatography on silica gel (20-30% of EtOAc in PE) to afford tert-butyl 3-[4-(2-nitro-5-pyrazol-1-yl-anilino)phenyl]azetidine-1-carboxylate (1.50 g, 92.3% yield) as a yellow oil. ¹H NMR (400 MHz, DMSO-d₆) δ=9.62 (s, 1H), 8.58 (d, J=2.4 Hz, 1H), 8.27 (d, J=9.6 Hz, 1H), 7.78 (d, J=1.6 Hz, 1H), 7.68 (d, J=2.4 Hz, 1H), 7.50-7.30 (m, 5H), 6.62-6.55 (m, 1H), 4.35-4.18 (m, 2H), 3.85 (s, 3H), 1.41 (s, 9H).

Step 3. To a stirred solution of 2-aminopyridine-3-carbaldehyde (561 mg, 4.59 mmol) and tert-butyl 3-[4-(2-nitro-5-pyrazol-1-yl-anilino)phenyl]azetidine-1-carboxylate (1.00 g, 2.30 mmol) in DMSO (15.0 mL) and methanol (3.00 mL), Na₂S₂O₄ (1.60 g, 9.20 mmol) was added and the reaction was stirred at 95° C. under O₂ for 12 hrs. The reaction mixture was diluted with EtOAc (50 mL), washed with water (50 mL) and brine (50 mL×2), dried over anhydrous sodium sulfate, filtered and the filtrate was concentrated to afford a residue. The crude product was purified by flash chromatography on silica gel (95% of EtOAc in PE) to afford tert-butyl 3-[4-[2-(2-amino-3-pyridyl)-6-pyrazol-1-yl-benzimidazol-1-yl]phenyl]azetidine-1-carboxylate (800 mg, 68.6% yield) as a yellow oil. ¹H NMR (400 MHz, CDCl₃) δ=8.06 (dd, J=2.0, 4.8 Hz, 1H), 7.94 (d, J=2.4 Hz, 1H), 7.88 (d, J=9.2 Hz, 1H), 7.71 (d, J=1.6 Hz, 1H), 7.64-7.57 (m, 2H), 7.50 (d, J=8.4 Hz, 2H), 7.35 (d, J=8.4 Hz, 2H), 7.06 (dd, J=2.0.7.6 Hz, 1H), 6.62 (s, 2H), 6.47 (t, J=2.0 Hz, 1H), 6.36 (dd, J=4.8, 8.0 Hz, 1H), 4.42 (t, J=8.6 Hz, 2H), 4.05 (dd, J=6.0, 8.4 Hz, 2H), 3.84 (s, 1H), 1.50 (s, 9H).

Step 4. To a stirred solution of tert-butyl 3-[4-[2-(2-amino-3-pyridyl)-6-pyrazol-1-yl-benzimidazol-1-yl]phenyl]azetidine-1-carboxylate (800 mg, 1.58 mmol) in DCM (4.00 mL), TFA (2.00 mL) was added and the reaction mixture was stirred at 20° C. under N₂ for 15 hrs. The reaction mixture was quenched with saturated aqueous NaHCO₃ solution (15 mL) and extracted with MeOH in DCM 10% solution (10 mL×3). The combined organic layers were dried over anhydrous sodium sulfate, filtered and the filtrate was concentrated to afford 3-[1-[4-(azetidin-3-yl)phenyl]-6-pyrazol-1-yl-benzimidazol-2-yl]pyridin-2-amine (240 mg, 37.4% yield) as a white solid. LCMS Rₜ=0.716 min in 1.5 min chromatography, purity 98.746%, MS ESI calcd. for 407.19 [M+H]⁺ 408.19, found 408.2.

Step 5. To a stirred solution of 3-[1-[4-(azetidin-3-yl)phenyl]-6-pyrazol-1-yl-benzimidazol-2-yl]pyridin-2-amine (240 mg, 0.590 mmol) and trans-methyl 4-(bromomethyl)cyclohexanecarboxylate (208 mg, 0.880 mmol) in DMSO (5.00 mL), DIEA (0.540 mL, 2.94 mmol) was added and the reaction mixture was stirred at 80° C. under N₂ for 15 hrs. The reaction mixture was diluted with EtOAc (50 mL), washed with water (50 mL) and brine (50 mL×2), dried over anhydrous sodium sulfate, filtered and the filtrate was concentrated to afford a residue. The crude product was purified by flash chromatography on silica gel (10% of methanol in DCM) to afford trans-methyl 4-[[3-[4-[2-(2-amino-3-pyridyl)-6-pyrazol-1-yl-benzimidazol-1-yl]phenyl]azetidin-1-yl]methyl]cyclohexanecarboxylate (100 mg, 30.2% yield) as a yellow oil. LCMS Rₜ=0.807 min in 1.5 min chromatography, purity 70.527%, MS ESI calcd. for 561.29 [M+Na]⁺ 584.29, found 584.4.

Step 6. To a stirred solution of trans-methyl 4-[[3-[4-[2-(2-amino-3-pyridyl)-6-pyrazol-1-yl-benzimidazol-1-yl]phenyl]azetidin-1-yl]methyl]cyclohexanecarboxylate (100 mg, 0.178 mmol) in THF (1.00 mL) and water (1.00 mL), LiOH·H₂O (21.0 mg, 0.530 mmol) was added and the reaction mixture was stirred at 25° C. under N₂ for 12 hrs. The reaction was concentrated and adjusted to pH ~5 with 1N HCl, and subsequently purified by prep-HPLC (column: Welch Xtimate C18 150×30 mm×5 μm, method: water (FA)-ACN, begin B: 10, end B: 40) to afford trans-4-[[3-[4-[2-(2-amino-3-pyridyl)-6-pyrazol-1-yl-benzimidazol-1-yl]phenyl]azetidin-1-yl]methyl]cyclohexanecarboxylic acid I-72 (43 mg, 43.9% yield) as a white solid. ¹H NMR (400 MHz, DMSO-d₆) δ=8.53 (d, J=2.4 Hz, 1H), 8.18 (s, 1H), 7.97 (dd, J=2.0, 4.8 Hz, 1H), 7.92-7.86 (m, 1H), 7.83 (d, J=2.0 Hz, 1H), 7.69 (d, J=2.0 Hz, 1H), 7.61-7.51 (m, 3H), 7.43 (d, J=8.4 Hz, 2H), 7.15 (dd, J=2.0, 7.6 Hz, 1H), 7.03 (s, 2H), 6.58-6.46 (m, 1H), 6.39 (dd, J=4.8, 7.6 Hz, 1H), 3.73-3.66 (m, 3H), 3.23-3.18 (m, 2H), 2.34 (d, J=6.4 Hz, 2H), 2.16-2.04 (m, 1H), 1.94-1.72 (m, 4H), 1.38-1.17 (m, 3H), 0.98-0.82 (m, 2H). HPLC Rₜ=3.021 min in 8 min chromatography, purity 99.147%. LCMS Rₜ=1.597 min in 4 min chromatography, purity 100%, MS ESI calcd. for 547.27 [M+H]⁺ 548.27, found 548.1.

### Example 64. Synthesis of (1r,4r)-4-((3-(4-(2-(2-aminopyridin-3-yl)-6-(pyridin-3-yl)-3H-imidazo[4,5-b]pyridin-3-yl)phenyl)azetidin-1-yl)methyl)cyclohexane-1-carboxylic acid (Compound I-73)

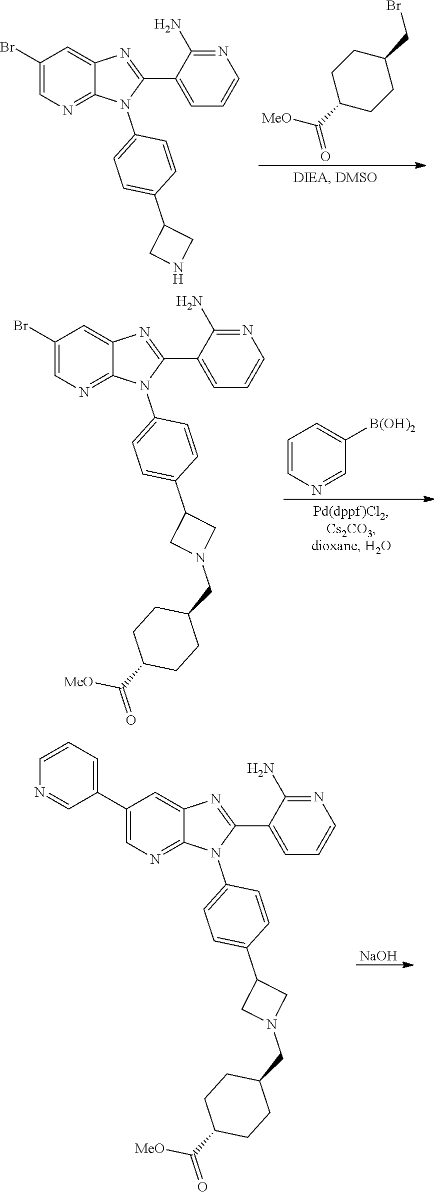

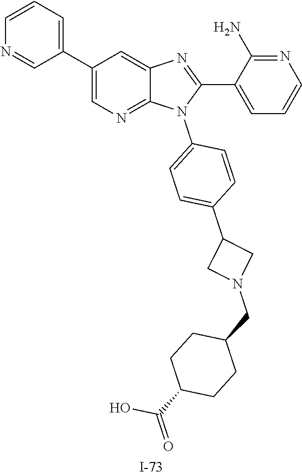

Step 1. To a solution of 3-[3-[4-(azetidin-3-yl)phenyl]-6-bromo-imidazo[4,5-b]pyridin-2-yl]pyridin-2-amine (430 mg, 1.02 mmol) in DMSO (3.00 mL), trans-methyl 4-(bromomethyl)cyclohexanecarboxylate (336 mg, 1.43 mmol) and DIEA were added and the reaction then stirred at 80° C. for 16 hrs under N₂. Water (100 mL) was added to the mixture and the whole extracted with EtOAc (80 mL×3). The organic layer was dried over Na₂SO₄ and concentrated in vacuo to provide the crude material. The crude was purified by flash column (0-30% of MeOH in DCM) to give trans-methyl 4-[[3-[4-[2-(2-amino-3-pyridyl)-6-bromo-imidazo[4,5-b]pyridin-3-yl]phenyl]azetidin-1-yl]methyl]cyclohexanecarboxylate (230 mg, 39.2% yield) as a yellow solid. ¹H NMR (400 MHz, DMSO-d₆) δ=8.53-8.47 (m, 1H), 8.44-8.38 (m, 1H), 8.05-7.96 (dd, J=1.6, 4.8 Hz, 1H), 7.54-7.34 (m, 4H), 7.27-7.21 (m, 1H), 6.92 (s, 1H), 6.46-6.36 (m, 1H), 3.80-3.48 (m, 5H), 3.15-3.00 (m, 1H), 2.31-2.13 (m, 3H), 1.93-1.84 (m, 2H), 1.83-1.76 (m, 2H), 1.36-1.11 (m, 4H), 1.00-0.82 (m, 2H).

Step 2. To a solution of trans-methyl 4-[[3-[4-[2-(2-amino-3-pyridyl)-6-bromo-imidazo[4,5-b]pyridin-3-yl]phenyl]azetidin-1-yl]methyl]cyclohexanecarboxylate (160 mg, 0.278 mmol) in 1,4-dioxane (3.00 mL) and water (1.00 mL), 3-pyridylboronic acid (48.0 mg, 0.389 mmol), Pd(dppf)Cl₂ (20.0 mg, 0.0278 mmol) and Cs₂CO₃ (272 mg, 0.834 mmol) were added at 25° C., and the mixture was stirred at 90° C. for 14 hrs under N₂. Water (30 mL) was added to the reaction mixture and the whole extracted with EtOAc (20 mL×3). The organic layer was dried over Na₂SO₄ and concentrated in vacuo to give the crude material. The crude was purified by flash column (0-30% of MeOH in DCM) to give trans-methyl 4-[[3-[4-[2-(2-amino-3-pyridyl)-6-(3-pyridyl)imidazo[4,5-b]pyridin-3-yl]phenyl]azetidin-1-yl]methyl]cyclohexanecarboxylate (100 mg, 62.7% yield) as a yellow solid. ¹H NMR (400 MHz, DMSO-d₆) δ=9.02 (d, J=1.2 Hz, 1H), 8.72-8.55 (m, 3H), 8.24-8.17 (m, 1H), 8.01 (dd, J=1.6, 4.4 Hz, 1H), 7.56-7.39 (m, 5H), 7.29-7.20 (m, 1H), 7.01 (s, 2H), 6.43 (dd, J=4.8, 7.6 Hz, 1H), 3.77-3.47 (m, 5H), 3.16-3.05 (m, 1H), 2.54 (s, 1H), 2.30-2.13 (m, 2H), 1.94-1.77 (m, 4H), 1.35-1.22 (m, 3H), 1.02-0.83 (m, 2H).

Step 3. To a solution of trans-methyl 4-[[3-[4-[2-(2-amino-3-pyridyl)-6-(3-pyridyl)imidazo[4,5-b]pyridin-3-yl]phenyl]azetidin-1-yl]methyl]cyclohexanecarboxylate (51.0 mg, 0.0882 mmol) in 1,4-dioxane (1.00 mL) and water (1.00 mL), NaOH (14.0 mg, 0.353 mmol) was added and the reaction mixture stirred at 30° C. for 3 hrs. The crude was purified by prep-HPLC (column: Welch Xtimate C18 150*30 mm*5 um, mobile phase: water(FA)-ACN, B %:0%-300%, 30 min) to give trans-4-[[3-[4-[2-(2-amino-3-pyridyl)-6-(3-pyridyl)imidazo[4,5-b]pyridin-3-yl]phenyl]azetidin-1-yl]methyl]cyclohexanecarboxylic acid I-73 (33.0 mg, 67.6% yield) as a yellow solid. ¹H NMR (400 MHz, DMSO-d₆) δ=9.02 (d, 0.1=1.6 Hz, 1H), 8.68 (d, J=2.0 Hz, 1H), 8.62 (dd, J=1.6, 4.8 Hz, 1H), 8.58 (d, J=2.0 Hz, 1H), 8.24-8.19 (m, 1H), 8.16 (s, 1H), 8.01 (dd, J=2.0, 4.8 Hz, 1H), 7.58-7.48 (m, 3H), 742 (d, J=8.4 Hz, 2H), 7.26 (dd, 1=2.0, 8.0 Hz, 1H), 7.02 (s, 2H), 6.43 (dd, J=4.4, 7.2 Hz, 1H), 3.75-3.61 (m, 3H), 3.19-3.14 (m, 2H), 2.34 (d, J=6.8 Hz, 2H), 2.15-2.02 (m, 1H), 1.93-1.73 (m, 4H), 1.34-1.20 (m, 3H), 0.98-0.85 (m, 2H). LCMS Rₜ=2.091 min in 4 min chromatography, purity 98.785%, MS ESI calcd. For 559.27 [M+H]⁺ 560.27, found 560.3. HPLC Rₜ=2.198 min in 8 min chromatography, purity 100%.

### Example 65. Synthesis of (1S,4r)-4-((S)-1-(3-(4-(2-(2-aminopyridin-3-yl)-5-phenyl-3H-imidazo[4,5-b]pyridin-3-yl)phenyl)azetidin-1-yl)-2-methylpropyl)cyclohexane-1-carboxylic acid (Compound I-74)

### Synthesis of methyl (1r,4r)-4-isobutyrylcyclohexane-1-carboxylate

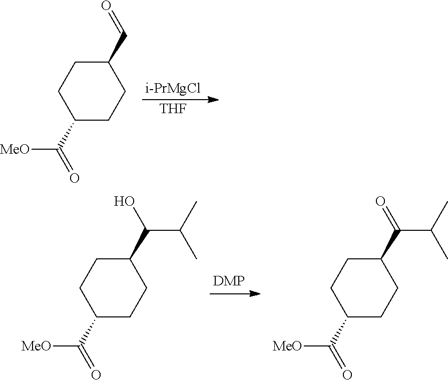

Step 1. To a solution of trans-methyl 4-formylcyclohexanecarboxylate (2.00 g, 11.8 mmol) in THF (20.0 mL), i-PrMgCl (6.50 mL, 12.9 mmol, 2M) was added at 0° C. under N₂, and the mixture was stirred at 25° C. for 2 hrs. Water was added to the mixture, and the whole extracted with EtOAc (20 mL×3). The organic phase was washed with brine (50 mL), dried with Na₂SO₄ and concentrated. The residue was purified by column (EtOAc in PE, 0-50%) to give trans-methyl 4-(1-hydroxy-2-methyl-propyl)cyclohexanecarboxylate (1.30 g, 51.6% yield) as a colorless oil. ¹H NMR (DMSO-d₆, 400 MHz) δ_{H}=4.18 (d, J=6.4 Hz, 1H), 3.62-3.55 (m, 3H), 2.83 (q, J=5.6 Hz, 1H), 2.25-2.12 (m, 1H), 1.95-1.84 (m, 3H), 1.69-1.60 (m, 1H), 1.59-1.51 (m, 1H), 1.34-1.20 (m, 3H), 1.18-1.09 (m, 1H), 1.03-0.94 (m, 1H), 0.82 (dd, J=6.8, 9.6 Hz, 6H).

Step 2. To a solution of trans-methyl 4-(1-hydroxy-2-methyl-propyl)cyclohexanecarboxylate (700 mg, 3.27 mmol) in DCM (10.0 mL), DMP (2.77 g, 6.53 mmol) was added at 0° C., and the mixture was stirred at 25° C. for 2 hrs. The mixture was quenched with 1:1 (1M Na₂S₂O₃:NaHCO₃), and further stirred for 30 mins. The mixture was extracted with DCM (20 mL×3), and the organic phase was washed with brine, dried with Na₂SO₄ and concentrated. The residue was purified by column (EtOAc in PE, 0-20%) to give methyl (1r,4r)-4-isobutyrylcyclohexane-1-carboxylate (500 mg, crude) as a white solid. ¹H NMR (DMSO-d₆, 400 MHz) δ_{H}=3.58 (s, 3H), 2.84-2.73 (m, 1H), 2.61-2.53 (m, 1H), 2.34-2.19 (m, 1H), 1.97-1.86 (m, 2H), 1.84-1.71 (m, 2H), 1.45-1.12 (m, 4H), 0.97 (d, J=6.8 Hz, 5H).

### Synthesis of (1S,4r)-4-((.%-1-(3-(4-(2-(2-aminopyridin-3-yl)-5-phenyl-3H-imidazo[4,5-b]pyridin-3-yl)phenyl)azetidin-1-yl)-2-methylpropyl)cyclohexane-1-carboxylic acid (Compound I-74)

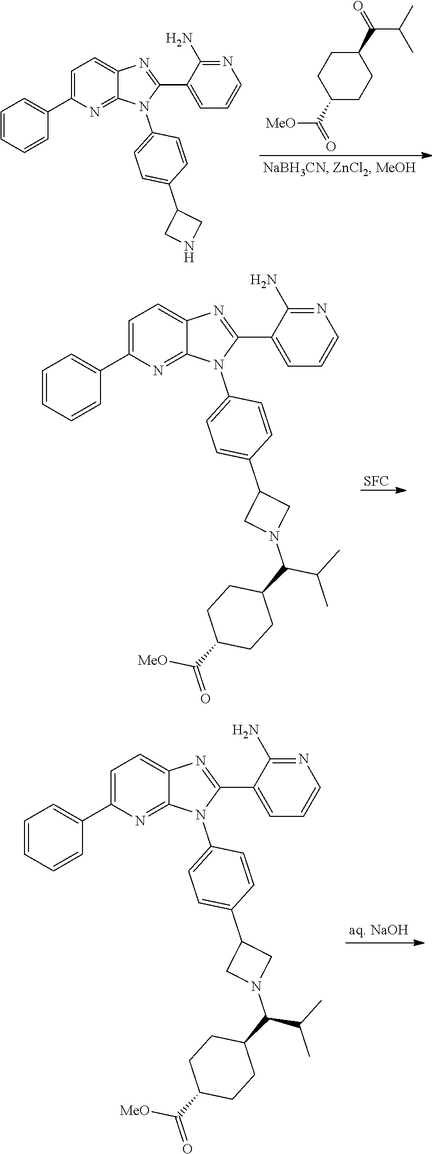 [SMILES: Nc1ncccc1-c1nc2ccc(-c3ccccc3)nc2n1-c1ccc(C2CNC2)cc1 ; COC(=O)[C@H]1CC[C@H](C(=O)C(C)C)CC1 ; COC(=O)[C@H]1CC[C@H](C(C(C)C)N2CC(c3ccc(-n4c(-c5cccnc5N)nc5ccc(-c6ccccc6)nc54)cc3)C2)CC1 ; "SFC" ; COC(=O)[C@H]1CC[C@H]([C@H](C(C)C)N2CC(c3ccc(-n4c(-c5cccnc5N)nc5ccc(-c6ccccc6)nc54)cc3)C2)CC1]

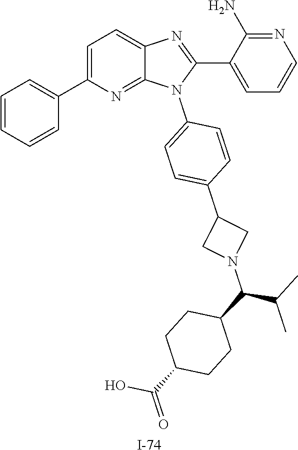

Step 1. To a solution of 3-[3-[4-(azetidin-3-yl)phenyl]-5-phenyl-imidazo[4,5-b]pyridin-2-yl]pyridin-2-amine (40.0 mg, 0.0956 mmol) in methanol (2.00 mL), methyl (1r,4r)-4-isobutyrylcyclohexane-1-carboxylate (81.0 mg, 0.382 mmol), NaBH₃CN (30.0 mg, 0.478 mmol) and ZnCl₂ (13.0 mg, 0.0956 mmol) were added and the mixture was stirred at 60° C. for 16 hrs. Water (20 mL) was added and the mixture was extracted with DCM (20 mL 3). The organic phase was washed with brine (10 mL), dried with Na₂SO₄ and concentrated. The residue was purified by column (MeOH in DCM, 0-10%) to give trans-methyl 4-[1-[3-[4-[2-(2-amino-3-pyridyl)-5-phenyl-imidazo[4,5-b]pyridin-3-yl]phenyl]azetidin-1-yl]-2-methyl-propyl]cyclohexanecarboxylate (20.0 mg, 34.0% yield) as a yellow solid. ¹H NMR (METHANOL-d, 400 MHz) δ_{H}=8.12 (d, J=8.4 Hz, 1H), 8.02-7.97 (m, 2H), 7.95 (dd, J=1.6, 5.2 Hz, 1H), 7.87 (d, J=8.4 Hz, 1H), 7.49-7.42 (m, 2H), 7.40-7.31 (m, 5H), 7.26 (dd, J=1.6, 7.6 Hz, 1H), 6.44 (dd, J=5.2, 7.6 Hz, 1H), 3.97-3.74 (m, 3H), 3.62 (s, 3H), 3.34 (s, 2H), 2.34-2.14 (m, 2H), 1.98-1.81 (m, 3H), 1.78-1.65 (m, 2H), 1.55-1.25 (m, 5H), 0.95 (dd, J=5.2, 6.8 Hz, 6H).

Step 2. Trans-methyl 4-[1-[3-[4-[2-(2-amino-3-pyridyl)-5-phenyl-imidazo[4,5-b]pyridin-3-yl]phenyl]azetidin-1-yl]-2-methyl-propyl]cyclohexanecarboxylate (100 mg, 0.163 mmol) was purified by SFC to give trans-methyl 4-[(1S)-1-[3-[4-[2-(2-amino-3-pyridyl)-5-phenyl-imidazo[4,5-b]pyridin-3-yl]phenyl]azetidin-1-yl]-2-methyl-propyl]cyclohexanecarboxylate (40.0 mg, 40.0% yield). LCMS Rₜ=0.702 min in 1 min chromatography, 5-95AB_1min_220&254_Agilent, purity 100%, MS ESI calcd. for 614.34 [M+H]⁺ 615.34, found 615.3.

Step 3. To a solution of trans-methyl 4-[(1S)-1-[3-[4-[2-(2-amino-3-pyridyl)-5-phenyl-imidazo[4,5-b]pyridin-3-yl]phenyl]azetidin-1-yl]-2-methyl-propyl]cyclohexanecarboxylate (40.0 mg, 0.0651 mmol) in THF (1.00 mL) and water (1.00 mL). NaOH (13.0 mg, 0.325 mmol) was added and the mixture was stirred at 35° C. for 16 hrs. To the mixture, NaOH (16.0 mg, 0.407 mmol) and MeOH (1.00 mL) were added, and the mixture was stirred at 35° C. for 8 hrs. The mixture was adjusted to pH ~7 and concentrated. The residue was purified by prep-HPLC (Welch Xtimate C18 150*30 mm*5 μm, water(FA)-ACN) to give trans-4-[(1S)-1-[3-[4-[2-(2-amino-3-pyridyl)-5-phenyl-imidazo[4,5-b]pyridin-3-yl]phenyl]azetidin-1-yl]-2-methyl-propyl]cyclohexanecarboxylic acid 1-74 (22.2 mg, 0.0370 mmol, 56.8% yield) as a yellow solid. ¹H NMR (DMSO-d₆, 400 MHz) δ_{H}=12.13-11.73 (m, 1H), 8.27 (d, J=84 Hz, 1H), 8.06-7.95 (m, 4H), 7.55-7.50 (m, 2H), 7.50-7.43 (m, 4H), 7.42-7.36 (m, 1H), 7.20 (dd, J=1.6, 7.6 Hz, 1H), 6.95 (s, 2H), 6.42 (dd, J=4.8, 7.6 Hz, 1H), 3.82-3.62 (m, 3H), 3.23-3.06 (m, 2H), 2.18-2.05 (m, 1H), 2.02-1.73 (m, 4H), 1.65 (br d, J=9.6 Hz, 2H), 1.47-1.34 (m, 1H), 1.33-1.18 (m, 4H), 0.90 (t, J=6.4 Hz, 6H). HPLC Rₜ=4.045 min in 8 min chromatography, 0-60AB_8 min.1cm, purity 100%. LCMS Rₜ=2.821 min in 4 min chromatography, 0-60AB_4 min_220&254_Shimadzu.1cm, purity 100%, MS ESI calcd. for 600.3 [M+H]⁺ 601.3, found 601.2.

### Example 66. Synthesis of (1s,4s)-4-((3-(4-(2-(2-aminopyridin-3-yl)-5-phenyl-311-imidazo[4,5-b]pyridin-3-yl)phenyl)azetidin-1-yl)methyl)-1-methylcyclohexane-1-carboxylic acid (Compound I-75)

### Synthesis of ethyl 4-(bromomethyl)-1-methylcyclohexane-1-carboxylate

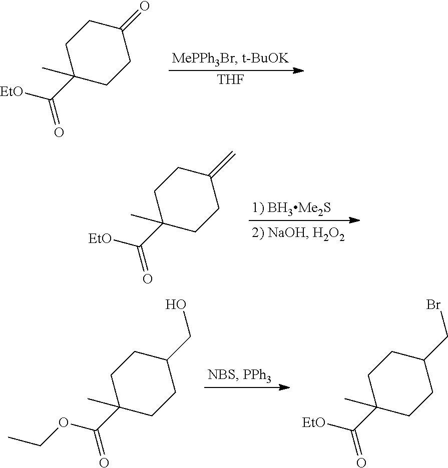

Step 1. To a solution of methyl(triphenyl)phosphonium bromide (9.89 g, 27.7 mmol) in THF (30.0 mL), t-BuOK (3.11 g, 27.7 mmol) was added, and the mixture stirred at 25° C. for 1 hr. Ethyl 1-methyl-4-oxocyclohexane-1-carboxylate (1.70 g, 9.23 mmol) was then added and the reaction stirred at 50° C. for 3 hrs. The reaction mixture was quenched by the addition of saturated aqueous ammonium chloride (100 mL), and subsequently extracted with EtOAc (300 mL×4). The organic layer was dried over Na₂SO₄ and concentrated in vacuo to provide the crude material. The crude material was purified by flash column (0-20% of EtOAc in PE) to give ethyl 1-methyl-4-methylenecyclohexane-1-carboxylate (1.40 g, 83.3% yield) as a yellow oil. ¹H NMR (400 MHz, CHLOROFORM-d) 8=4 64 (s, 2H), 4.18 (q, J=7.2 Hz, 2H), 2.25-2.12 (m, 6H), 1.38-1.31 (m, 2H), 1.28 (t, J=7.2 Hz, 3H), 1.20 (s, 3H).

Step 2. To a solution of ethyl 1-methyl-4-methylene-cyclohexanecarboxylate (1.30 g, 7.13 mmol) in THF (30.0 mL), BH₃·Me₂S (2.10 mL, 21.4 mmol) was added slowly at 0° C. under N₂ atmosphere. After addition, the mixture was stirred at 25° C. for 2 hrs, and then reaction mixture was quenched by MeOH (10 mL) at 0° C. under N₂ atmosphere Aqueous NaOH (3 M, 2 mL) and H₂O₂ (2.43 g, 71.3 mmol) were then added to the mixture, and the mixture was stirred at 25° C. for 2 hrs. Saturated aq. Na₂S₂O₃ (100 mL) was added, and the reaction mixture was then adjusted to pH ~5 by aq. HCl (4 M). The resulting mixture was extracted with EtOAc (60 mL 3). The combined organic phase was washed with brine (30 mL), water (30 mL), dried over anhydrous Na₂SO₄, filtered and concentrated. The residue was purified by flash silica gel chromatography (eluent of 0-50% EtOAc in PE) providing ethyl 4-(hydroxymethyl)-1-methylcyclohexane-1-carboxylate (1.00 g, 70.0% yield) as a colorless oil. ¹H NMR (400 MHz, CHLOROFORM-d) δ=4.17-4.09 (m, 2H), 3.50 (t, J=5.6 Hz, 1H), 3.43 (t, J=5.6 Hz, 1H), 2.28-2.19 (m, 1H), 1.75-1.62 (m, 4H), 1.54-1.39 (m, 1H), 1.27-1.14 (m, 8H), 1.08-0.98 (m, 1H).

Step 3. To a solution of ethyl 4-(hydroxymethyl)-1-methylcyclohexane-1-carboxylate (800 mg, 3.99 mmol) in DCM (15 mL), NBS (1.07 g, 5.99 mmol) and PPh₃ (1.57 g, 5.99 mmol) were added at 0° C., under N₂ atmosphere, and the mixture was stirred at 25° C. for 16 hrs. Water (20 mL) was added to the residue, and the resulting mixture was extracted with DCM (20 mL×3). The combined organic phase was washed with brine (10 mL), water (10 mL), dried over anhydrous Na₂SO₄, filtered and concentrated. The residue was purified by flash silica gel chromatography (eluent with 0-10% of EtOAc in PE) to provide ethyl 4-(bromomethyl)-1-methylcyclohexane-1-carboxylate (700 mg, 66.6% yield) as a colorless oil. ¹H NMR (400 MHz, CHLOROFORM-d) δ=4.11-4.02 (m, 2H), 3.27 (d, J=6.4 Hz, 1H), 3.18 (d, J=6.4 Hz, 1H), 2.20-2.13 (m, 1H), 1.77-1.61 (m, 4H), 1.60-1.51 (m, 1H), 1.24-1.07 (m, 8H), 1.07-1.00 (m, 1H).

### Synthesis of (1s,4s)-4-((3-(4-(2-(2-aminopyridin-3-yl)-5-phenyl-3H-imidazo[4,5-b]pyridin-3-yl)phenyl)azedin-1-yl)methyl)-1-methylcyclohexane-1-carboxylic acid (Compound I-75)

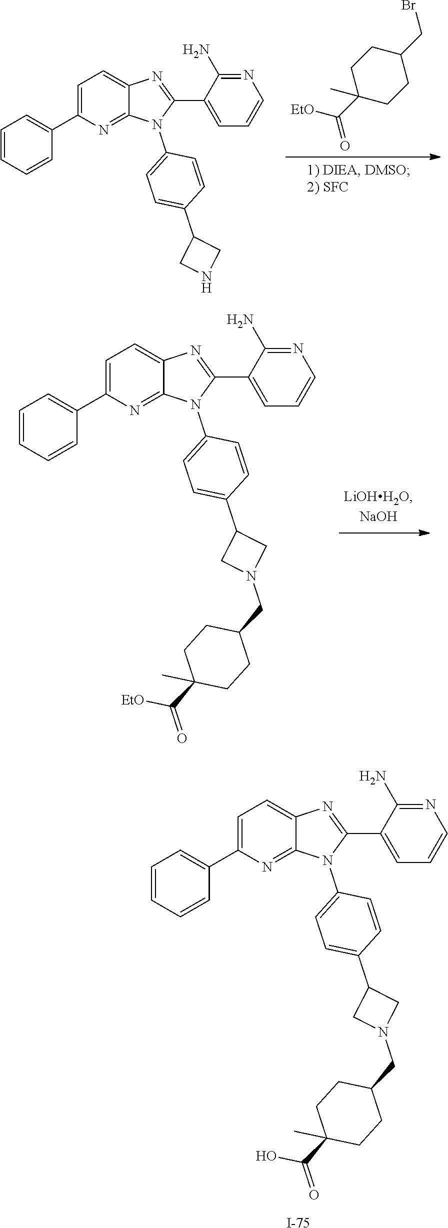

Step 1. To a solution of ethyl 4-(bromomethyl)-1-methylcyclohexane-1-carboxylate (0.700 g, 2.66 mmol) in DMSO (4.00 mL), 3-(3-(4-(azetidin-3-yl)phenyl)-5-phenyl-3H-imidazo[4,5-b]pyridin-2-yl)pyridin-2-amine (1.11 g, 2.66 mmol) and DIEA (1.50 mL, 7.98 mmol) were added at 25° C., and the mixture was heated to, and stirred at 80° C. for 16 hrs. The reaction mixture was concentrated, and water (30 mL) was added to the residue. The resulting mixture was extracted with EtOAc (20 mL×3). The combined organic phase was washed with brine (10 mL), water (10 mL), dried over anhydrous Na₂SO₄, filtered and concentrated. The residue was purified by flash silica gel chromatography (eluent of 0-5% of MeOH in DCM) and further purified by SFC (column: DAICEL CHIRALPAK AD (250 mm*30 mm, 10 μm); condition: CO₂-EtOH (0.1% NH₃H₂O); begin b: 50-50) to provide methyl (1s,4s)-4-((3-(4-(2-(2-aminopyridin-3-yl)-5-phenyl-3H-imidazo[4,5-b]pyridin-3-yl)phenyl)azetidin-1-yl)methyl)-1-methylcyclohexane-1-carboxylate (80.0 mg, 26.7% yield) as a yellow oil. ¹H NMR (400 MHz, CHLOROFORM-d) δ=8.12 (d, J=8.4 Hz, 1H), 8.07 (dd, J=4.8, 1.6 Hz, 1H), 8.02 (d, J=7.6 Hz, 2H), 7.80 (d, J=8.4 Hz, 1H), 7.47-7.34 (m, 7H), 7.12 (dd, J=7.6, 1.6 Hz, 1H), 6.58 (s, 2H), 6.40 (dd, J=7.6, 4.8 Hz, 1H), 4.12 (q, J=7 2 Hz, 2H), 3.80 (s, 3H), 3.24-3.14 (m, 2H), 2.42 (d, J=6.8 Hz, 2H), 1.74-1.62 (m, 6H), 1.46-1.34 (m, 1H), 1.27-1.18 (m, 8H).

Step 2. To a solution of methyl (1s,4s)-4-((3-(4-(2-(2-aminopyridin-3-yl)-5-phenyl-3H-imidazo[4,5-b]pyridin-3-yl)phenyl)azetidin-1-yl)methyl)-1-methylcyclohexane-1-carboxylate (70.0 mg, 0.117 mmol) in THF (3.00 mL) and water (1.00 mL), LiOH·H₂O (48.0 mg, 1.17 mmol), NaOH (47.0 mg, 1.17 mmol) and methanol (0.5 mL) were added at 25° C., and the mixture was stirred at 60° C. for 16 hrs. The residue was purified by prep-HPLC (column: F-Prepulite XP tC 18 40*200 mm*7 μm; condition: water (NH₄HCO₃)-ACN; begin b: 0-40) to provide (1s,4s)-4-((3-(4-(2-(2-aminopyridin-3-yl)-5-phenyl-3H-imidazo[4,5-b]pyridin-3-yl)phenyl)azetidin-1-yl)methyl)-1-methylcyclohexane-1-carboxylic acid 1-75 (8.30 mg, 12.4% yield) as a white solid. ¹H NMR (400 MHz, DMSO-d₆) δ=8.27 (d, J=8.4 Hz, 1H), 8.06-7.96 (m, 4H), 7.55-7.37 (m, 7H), 7.20 (dd, J=7.6, 1.6 Hz, 1H), 6.96 (s, 2H), 6.43 (dd, J=7.6, 4.8 Hz, 1H), 3.72-3.59 (m, 3H), 3.12-3.06 (m, 2H), 2.26 (d, J=6.4 Hz, 2H), 2.05 (d, J=12.0 Hz, 2H), 1.63 (d, J=11.6 Hz, 2H), 1.27-1.22 (m, 1H), 1.13-1.01 (m, 5H), 1.00-0.88 (m, 2H). HPLC Rt=2.139 min in 8 min chromatography, purity 100%. LCMS Rt=0.758 min in 1.5 min chromatography, purity 97.4%, MS ESI calcd. for 572.29, [M+H]⁺ 573.29, found 573.3.

### Example 67. Synthesis of 4-((4-(4-(2-(2-aminopyridin-3-yl)-5-phenyl-3H-imidazo[4,5-b]pyridin-3-yl)phenyl)piperidin-1-yl)methyl)cyclohexane-1-carboxylic acid (Compound I-76)

### Synthesis of methyl 4-formylcyclohexane-1-carboxylate

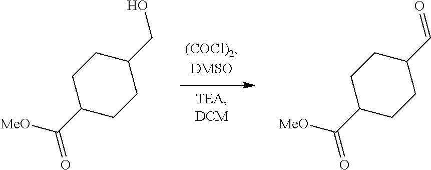 [SMILES: COC(=O)C1CCC(CO)CC1 ; COC(=O)C1CCC(C=O)CC1]

Step 1. To a solution of DMSO (0.300 mL, 4.18 mmol) in DCM (3.00 mL), (COCl)₂ (0.180 mL, 2.09 mmol) was added and the reaction was stirred at −78° C. for 5 mins. Methyl 4-(hydroxymethyl)cyclohexane-1-carboxylate (0.300 g, 1.74 mmol) in DCM (3.00 mL) was added at −78° C. After stirring for 30 mins, TEA (1.20 mL, 8.71 mmol) was added over 15 mins, and then the cooling bath was removed. The reaction mixture was quenched with 1 M hydrochloric acid aqueous solution (15 mL) at −10° C., and the layers were separated. The organic layer was washed with water (25 mL) and brine (10 mL), dried over anhydrous sodium sulfate and concentrated to give methyl 4-formylcyclohexane-1-carboxylate (0.30 g, 101.18% yield) as a yellow oil. ¹H NMR (400 MHz, DMSO-d₆) δ=9.55 (s, 1H), 3.61-3.55 (m, 3H), 2.27-2.20 (m, 2H), 1.97-1.92 (m, 4H), 1.36-1.23 (m, 4H).

### Synthesis of 4-((4-(4-(2-(2-aminopyridin-3-yl)-5-phenyl-3H-imidazo[4,5-b]pyridin-3-yl)phenyl)piperidin-1-yl)methyl)cyclohexane-1-carboxylic acid (Compound I-76)

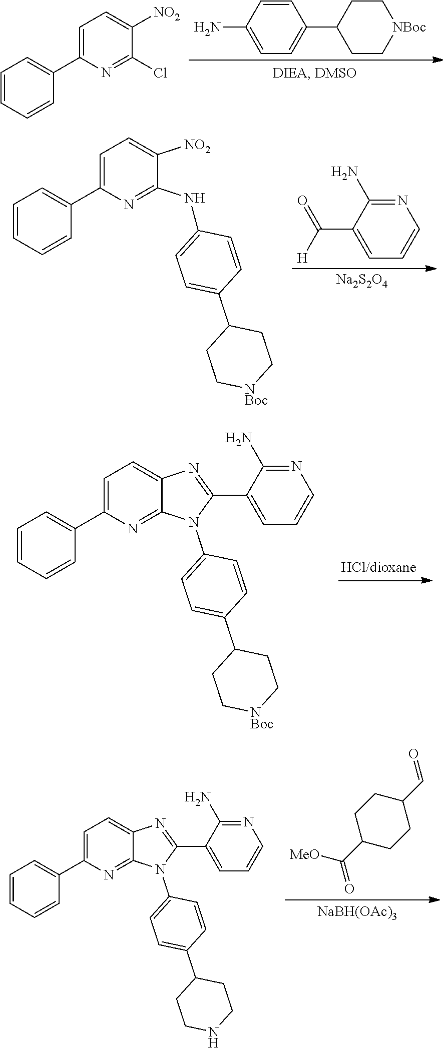

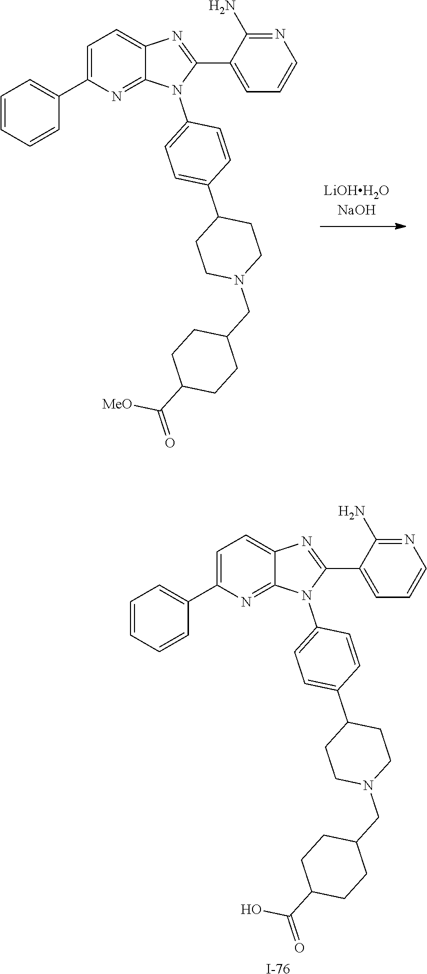

Step 1. To a solution of tert-butyl 4-(4-aminophenyl)piperidine-1-carboxylate (2.83 g, 10.2 mmol) in DMSO (20.0 mL), DIEA (3.30 g, 25.6 mmol) and 2-chloro-3-nitro-6-phenyl-pyridine (2.00 g, 8.52 mmol) were added at 25° C., and the mixture was stirred at 80° C. for 14 hrs. EtOAc (50 mL) was added to the reaction mixture. The resulting suspension was washed with brine (40 mL×3), dried over anhydrous Na₂SO₄, filtered and concentrated. The mixture was triturated with ACN (50 mL). The reaction mixture was filtered and the filter cake was concentrated to give tert-butyl 4-[4-[(3-nitro-6-phenyl-2-pyridyl)amino]phenyl]piperidine-1-carboxylate (3.30 g, 81.6% yield) as a red solid. LCMS Rₜ=1.877 min in 2 min chromatography, purity 62.7%, MS ESI calcd. for 474.23, [M+H]⁺ 475.23, found 475.3.

Step 2. To a solution of tert-butyl 4-[4-[(3-nitro-6-phenyl-2-pyridyl)amino]phenyl]piperidine-1-carboxylate (2.80 g, 5.90 mmol) and 2-aminopyridine-3-carbaldehyde (1.44 g, 11.8 mmol) in DMSO (28.0 mL) and methanol (2.00 mL), Na₂S₂O₄ (5.96 g, 34.2 mmol) was added under N₂, and the mixture was stirred at 100° C. for 12 hrs. The reaction mixture was concentrated. Water (40 mL) was added to the residue and the resulting mixture was extracted with EtOAc (60 mL×3). The combined organic phase was washed with brine (50 mL), dried over anhydrous Na₂SO₄, filtered and concentrated. The residue was purified by flash silica gel chromatography (eluent of 0-52% EtOAc in PE) to give tert-butyl 4-[4-[2-(2-amino-3-pyridyl)-5-phenyl-imidazo[4,5-b]pyridin-3-yl]phenyl]piperidine-1-carboxylate (1.23 g, 38.1% yield) as a yellow solid. LCMS Rₜ=0.885 min in 1.5 min chromatography, purity 25.73%, MS ESI calcd. for 546.27, [M+H]⁺ 546.27, found 547.7.

Step 3. To a solution of tert-butyl 4-[4-[2-(2-amino-3-pyridyl)-5-phenyl-imidazo[4,5-b]pyridin-3-yl]phenyl]piperidine-1-carboxylate (0.400 g, 0.732 mmol) in 1,4-dioxane (2.00 mL), HCl/dioxane (26.0 ml, 52.2 mmol) was added and the mixture was stirred at 25° C. for 12 hrs. The reaction mixture was concentrated to give 3-[5-phenyl-3-[4-(4-piperidyl)phenyl]imidazo[4,5-b]pyridin-2-yl]pyridin-2-amine (500 mg, crude) as a yellow solid. LCMS Rₜ=0.618 min in 2.0 min chromatography, purity 94.6%, MS ESI calcd. for 446.22, [M+H]⁺ 447.22, found 447.2.

Step 4. To a solution of 3-[5-phenyl-3-[4-(4-piperidyl)phenyl]imidazo[4,5-b]pyridin-2-yl]pyridin-2-amine (100 mg, 0.224 mmol) in DCM (3.00 mL), TEA (pH ~7) and methyl 4-formylcyclohexane-1-carboxylate (95.0 mg, 0.560 mmol) were added and the mixture (pH ~7) was stirred at 25° C. for 0.5 hrs. NaBH(OAc). (237 mg, 1.12 mmol) was subsequently added and the mixture was stirred at 25° C. for 2 hrs. Water (20 mL) was added to the mixture and the mixture was extracted with DCM (30 mL×3). The organic phase was washed with brine (10 mL×2), dried with Na₂SO₄ and concentrated. The mixture was purified by column (MeOH in DCM, 0-10%) to give methyl 4-((4-(4-(2-(2-aminopyridin-3-yl)-5-phenyl-3H-imidazo[4,5-b]pyridin-3-yl)phenyl)piperidin-1-yl)methyl)cyclohexane-1-carboxylate (160 mg, crude) as a yellow oil.

LCMS Rₜ=0.412 min in 1 min chromatography, purity 42.95%, MS ESI calcd. for 600.32, [M+H]⁺ 601.32, found 601.3.

Step 5. To a solution of methyl 4-((4-(4-(2-(2-aminopyridin-3-yl)-5-phenyl-3H-imidazo[4,5-b]pyridin-3-yl)phenyl)piperidin-1-yl)methyl)cyclohexane-1-carboxylate (160 mg, 0.266 mmol) in THF (2.00 mL), water (1.00 mL) and methanol (0.500 mL), LiOH·H₂O (34.0 mg, 0.799 mmol) was added and the mixture was stirred at 25° C. for 1 hr. Methanol (1.00 mL) and NaOH (32.0 mg, 0.799 mmol) were added and the mixture was stirred at 25° C. for 1 hr. The mixture was filtered and the residue was purified by prep-HPLC (Column: Welch Xtimate C18 40*200 mm 7 μm; mobile phase: water(FA)-ACN; B %: 0%-36%; 25 min) to give 4-((4-(4-(2-(2-aminopyridin-3-yl)-5-phenyl-3H-imidazo[4,5-b]pyridin-3-yl)phenyl)piperidin-1-yl)methyl)cyclohexane-1-carboxylic acid I-76 (11.0 mg, 7.04% yield) as a yellow solid. ¹H NMR (DMSO-d₆, 400 MHz) β_{H}=8.26 (d, J=8.4 Hz, 1H), 8.06-7.93 (m, 4H), 7.53-7.33 (m, 7H), 7.16 (dd, J=2.0, 8.0 Hz, 1H), 6.96 (s, 2H), 6.40 (dd, J=4.4, 7.6 Hz, 1H), 2.95 (d, J=10.8 Hz, 2H), 2.65-2.53 (m, 2H), 2.17-2.06 (m, 3H), 2.03-1.79 (m, 7H), 1.76-1.62 (m, 2H), 1.58-1.40 (m, 1H), 1.37-1.19 (m, 2H), 0.94-0.81 (m, 2H). HPLC Rₜ=3.726 min in 8 min chromatography, purity 97.22%. LCMS Rₜ=2.103 min in 4 min chromatography, purity 99.50%, MS ESI calcd. for 586.31, [M+H]⁺ 587.31, found 587.3.

### Example 68. Synthesis of 4-((3-(4-(2-(2-aminopyridin-3-yl)-5-phenyl-3H-imidazo[4,5-b]pyridin-3-yl)phenyl)piperidin-1-yl)methyl)cyclohexane-1-carboxylic acid (Compound I-77)

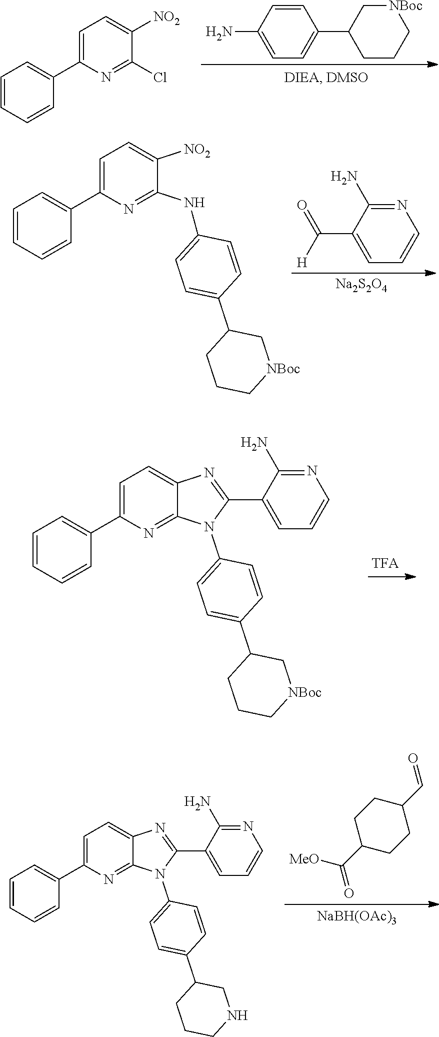

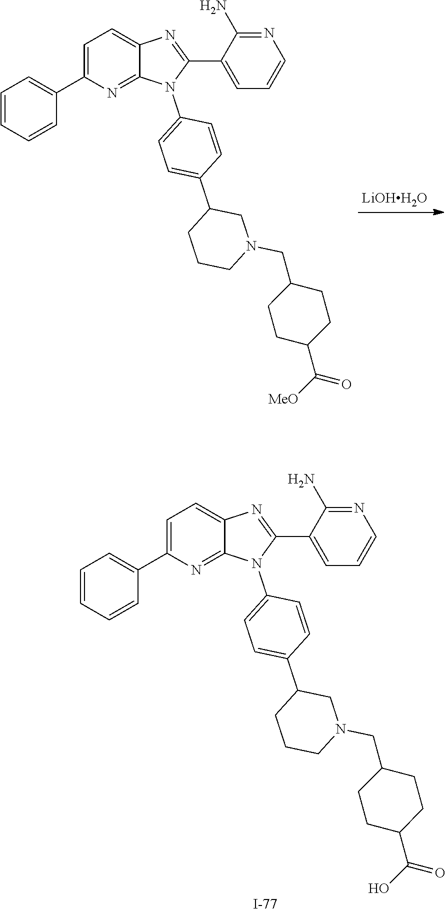

Step 1. A mixture of 2-chloro-3-nitro-6-phenyl-pyridine (3.40 g, 14.5 mmol), tert-butyl 3-(4-aminophenyl)piperidine-1-carboxylate (4.41 g, 15.9 mmol) and DIEA(7.80 mL, 43.5 mmol) in DMSO (50.0 L) was stirred at 90° C. for 16 hrs under N₂. Water (200 mL) was added to the mixture and the mixture was extracted with EtOAc (200 mL×3). The organic phase was washed with brine (50 mL×3), dried with Na₂SO₄ and concentrated. The residue was purified by column (EtOAc in PE, 0-30%) to give tert-butyl 3-[4-[(3-nitro-6-phenyl-2-pyridyl)amino]phenyl]piperidine-1-carboxylate (7.00 g, crude) as a red solid. ¹H NMR (CDCl₃ 400 MHz) δ_{H}=10.30 (s, 1H), 8.60 (d, J=8.8 Hz, 1H), 8.11-8.01 (m, 2H), 7.73 (d, J=8.4 Hz, 2H), 7.56-7.47 (m, 3H), 7.31 (dd, J=3.2, 8.5 Hz, 3H), 4.33-4.09 (m, 2H), 2.87-2.66 (m, 3H), 2.14-2.00 (m, 1H), 1.84-1.75 (m, 1H), 1.59 (br s, 2H), 1.50 (s, 9H).

Step 2. To a solution of tert-butyl 3-[4-[(3-nitro-6-phenyl-2-pyridyl)amino]phenyl]piperidine-1-carboxylate (3.00 g, 6.32 mmol) and 2-aminopyridine-3-carbaldehyde (1.04 g, 8.53 mmol) in DMSO (30.0 mL) and methanol (3.00 mL), Na₂S₂O₄ (3.30 g, 19.0 mmol) was added under N₂ and the mixture was stirred at 100° C. for 12 hrs. The reaction mixture was concentrated. Aq. LiCl (30 mL) was added to the residue and the resulting mixture was extracted with EtOAc (30 mL×3). The combined organic phase was washed with brine (30 mL), dried over anhydrous Na₂SO₄, filtered and concentrated. The residue was purified by flash silica gel chromatography (eluent of 0-99% EtOAc in PE) to give tert-butyl 3-[4-[2-(2-amino-3-pyridyl)-5-phenyl-imidazo[4,5-b]pyridin-3-yl]phenyl]piperidine-1-carboxylate (2.00 g, 57.9% yield) as a yellow solid. ¹H NMR (DMSO-d₆ 400 MHz) δ_{H}=8.26 (d, J=8.4 Hz, 1H), 8.08-7.92 (m, 4H), 7.53-7.34 (m, 7H), 7.17 (dd, J=1.6, 7.5 Hz, 1H), 6.96 (br s, 2H), 6.41 (dd, J=4.8, 7.7 Hz, 1H), 4.12-3.85 (m, 2H), 2.95-2.60 (m, 3H), 1.95 (br s, 1H), 1.76-1.67 (m, 2H), 1.47 (br d, J=10.4 Hz, 1H), 1.45 (s, 9H).

Step 3. To a solution of tert-butyl 3-[4-[2-(2-amino-3-pyridyl)-5-phenyl-imidazo[4,5-b]pyridin-3-yl]phenyl]piperidine-1-carboxylate (500 mg, 0.915 mmol) in DCM (2.00 mL), TFA (2.00 mL, 1.83 mmol) was added and the mixture was stirred for 2 hrs. The mixture was concentrated to give 3-[5-phenyl-3-[4-(3-piperidyl)phenyl]imidazo[4,5-b]pyridin-2-yl]pyridin-2-amine (400 mg, 97.9% yield) as a yellow oil. LCMS Rₜ=3.256 min in 4 min chromatography, Xtimate C18 2.1*30 mm,3 um, purity 98.65%, MS ESI calcd. For 446.56 [M+H]⁺ 447.56, found 447.1.

Step 4. To a solution of 3-[5-phenyl-3-[4-(3-piperidyl)phenyl]imidazo[4,5-b]pyridin-2-yl]pyridin-2-anine (200 mg, 0.448 mmol) in DCM (10.0 mL), TEA (pH ~7), methyl 4-formylcyclohexanecarboxylate (381 mg, 2.24 mmol) were added and the mixture (pH ~7) was stirred at 25° C. for 16 hrs. NaBH(OAc)₃ (475 mg, 2.24 mmol) was added and the mixture was stirred at 25° C. for 16 hrs. Water (10 mL) was added and the mixture was extracted with EtOAc (30 mL×3). The organic phase was washed with brine (10 mL×2), dried with Na₂SO₄ and concentrated. The mixture was purified by column (MeOH in DCM, 0-20%) to give methyl 4-[[3-[4-[2-(2-amino-3-pyridyl)-5-phenyl-imidazo[4,5-b]pyridin-3-yl]phenyl]-1-piperidyl]methyl]cyclohexanecarboxylate (100 mg, 32.7% yield) as a yellow solid. LCMS Rₜ=0.713 min in 1 min chromatography, Xtimate C18 2.1*30 mm,3 um, purity 94.25%, MS ESI calcd. for 600.77 [M+H]⁺ 601.77, found 601.3.

Step 5. To a solution of methyl 4-[[3-[4-[2-(2-amino-3-pyridyl)-5-phenyl-imidazo[4,5-b]pyridin-3-yl]phenyl]-1-piperidyl]methyl]cyclohexanecarboxylate (100 mg, 0.166 mmol) in THF (1.00 mL) and water (1.00 mL), LiOH·H₂O (21.0 mg, 0.499 mmol) was added and the mixture was stirred at 25° C. for 16 hrs Methanol (1 mL) was added to the mixture, and the mixture was stirred at 25° C. for 2 hrs and subsequently concentrated. The residue was dissolved with DMSO and 1M HCL, purified by prep-HPLC (Xtimate C18 150*40 mm*5 μm, water(FA)-ACN) to give 4-((3-(4-(2-(2-aminopyridin-3-yl)-5-phenyl-3H-imidazo[4,5-b]pyridin-3-yl)phenyl)piperidin-1-yl)methyl)cyclohexane-1-carboxylic acid I-77 (31.0 mg, 29.6% yield) as a yellow solid. ¹H NMR (DMSO-d₆, 400 MHz) δ_{H}=8.27 (d, J=8.4 Hz, 1H), 8.07-7.95 (m, 4H), 7.52-7.38 (m, 7H), 7.17 (dd, J=1.6, 7.6 Hz, 1H), 6.96 (s, 2H), 6.41 (dd, J=4.8, 7.6 Hz, 1H), 2.93 (br d, J=11.2 Hz, 1H), 2.90-2.81 (m, 2H), 2.20-2.11 (m, 3H), 1.99-1.77 (m, 7H), 1.76-1.70 (m, 1H), 1.65-1.58 (m, 1H), 1.53-1.44 (m, 2H), 1.36-1.23 (m, 2H), 0.88 (q, J=11.2 Hz, 2H). HPLC Rₜ=3.846 in 8 min chromatography, 0-60AB, purity 94.1%. LCMS Rₜ=2.921 min in 4 min chromatography, Xtimate C18 2.1*30 mm,3 μm, purity 100%, MS ESI calcd. for 586.74 [M+H]⁺ 587.74, found 587.6.

### Example 69. Synthesis of 4-((3-(4-(2-(2-aminopyridin-3-yl)-5-phenyl-3H-imidazo[4,5-b]pyridin-3-yl)phenyl)pyrrolidin-1-yl)methyl)cyclohexane-1-carboxylic acid (Compound I-78)

### Synthesis of tert-butyl 3-(4-aminophenyl)pyrrolidine-1-carboxylate

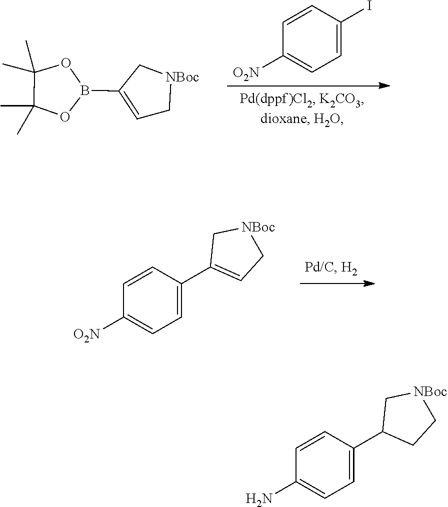 [SMILES: CC(C)(C)OC(=O)N1CC=C(B2OC(C)(C)C(C)(C)O2)C1 ; O=[N+]([O-])c1ccc(I)cc1 ; CC(C)(C)OC(=O)N1CC=C(c2ccc([N+](=O)[O-])cc2)C1 ; CC(C)(C)OC(=O)N1CCC(c2ccc(N)cc2)C1]

Step 1. To a solution of tert-butyl 3-(4,4,5,5-tetramethyl-1,3,2-dioxaborolan-2-yl)-2,5-dihydropyrrole-1-carboxylate (5.00 g, 16.9 mmol) in 1,4-dioxane (60.0 mL) and water (15.0 mL), 1-iodo-4-nitro-benzene (5.06 g, 20.3 mmol), Pd(dppf)Cl₂ (1.24 g, 1.69 mmol) and K₂CO₄ (5.85 g, 42.3 mmol) were added at 25° C. under N₂, and the mixture was stirred at 80° C. for 16 hrs. The mixture was filtered, water (500 mL) was added to the filtrate, and the mixture was extracted with EtOAc (500 mL×3). The organic phase was washed with brine (500 mL), dried over Na₂SO₄, filtered, and concentrated under reduced pressure to provide the crude material. The crude was purified by flash chromatography eluting with PE/EtOAc (from 0% to 20% of EtOAc in PE) to give tert-butyl 3-(4-nitrophenyl)-2,5-dihydropyrrole-1-carboxylate (2.50 g, 50.8% yield) as a yellow solid ¹H NMR (400 MHz, DMSO-d₆) δ=8.22 (d, J=8.8 Hz, 2H), 7.74 (dd, J=4.8, 8.8 Hz, 2H), 6.71 (d, J=14.6 Hz, 1H), 4.50-4.43 (m, 2H), 4.26 (d, J=3.8 Hz, 2H), 1.45 (d, J=5.8 Hz, 9H).

Step 2. To a solution of tert-butyl 3-(4-nitrophenyl)-2,5-dihydropyrrole-1-carboxylate (1.00 g, 3.44 mmol) in ethyl acetate (40.0 mL), Pd/C (100 mg, 10%, wet) was added at 25° C. The reaction was degassed and purged with Ar (×3), and subsequently degassed and purged with H₂ (×3), and stirred under H₂ (50 psi) for 16 hrs at 50° C. The reaction mixture was filtered, and the cake was washed with MeOH (20 mL×3). The combined filtrate was concentrated in vacuo to give tert-butyl 3-(4-aminophenyl)pyrrolidine-1-carboxylate (700 mg, 77.5% yield) as a yellow solid. ¹H NMR (400 MHz, CHLOROFORM-d) δ=7.03 (d, J=8.0 Hz, 2H), 6.65 (d, J=8.0 Hz, 2H), 3.92-3.13 (m, 5H), 2.26-2.12 (m, 1H), 1.99-1.84 (m, 1H), 1.48 (s, 9H).

### Synthesis of 4-((3-(4-(2-(2-aminopyridin-3-yl)-5-phenyl-3H-imidazo[4,5-b]pyridin-3-yl)phenyl)pyrrolidin-1-yl)methyl)cyclohexane-1-carbocylic acid (Compound I-78)

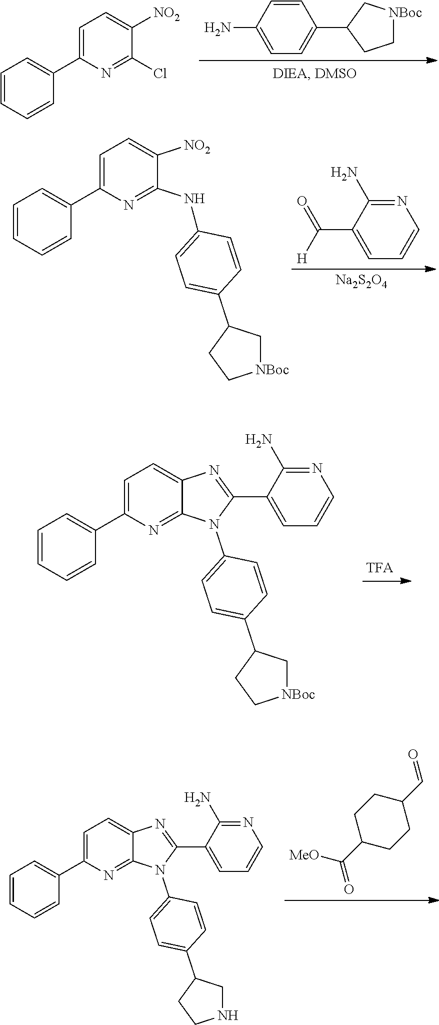

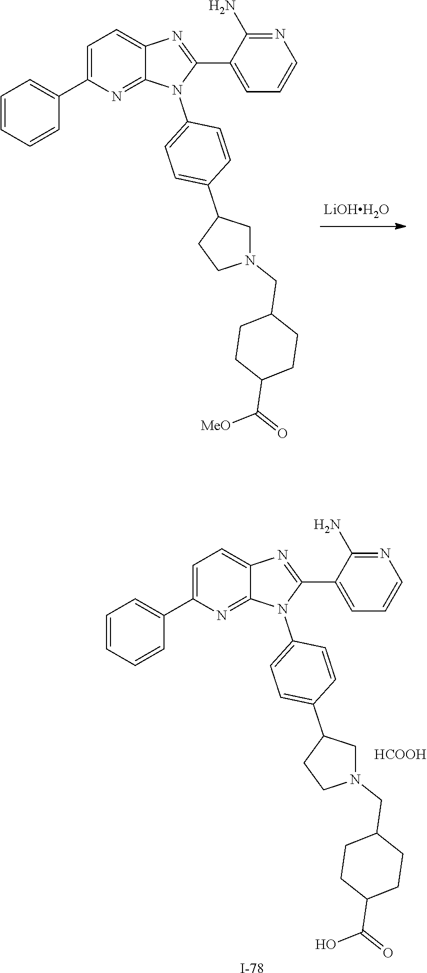

Step 1. To a solution of 2-chloro-3-nitro-6-phenyl-pyridine (1.90 g, 8.10 mmol) in DMSO (30.0 mL), tert-butyl 3-(4-aminophenyl)pyrrolidine-1-carboxylate (2.55 g, 9.72 mmol) and DIEA (3.70 mL, 20.2 mmol) were added at 25° C. under N₂, and the mixture was stirred at 90° C. for 16 hrs. Water (300 mL) was added, and the mixture was extracted with EtOAc (300 mL, 3). The organic phase was washed with brine (300 mL), dried over Na₂SO₄, filtered, and concentrated under reduced pressure to provide the crude material. The crude was purified by flash chromatography eluting with PE/EtOAc (from 0% to 30% of EtOAc in PE) to give tert-butyl 3-[4-[(3-nitro-6-phenyl-2-pyridyl)amino]phenyl]pyrrolidine-1-carboxylate (3.24 g, 87.0% yield) as a red oil. ¹H NMR (400 MHz, DMSO-d₆) δ=10.10 (s, 1H), 8.60 (d, J=8.8 Hz, 1H), 8.09 (dd, J=2.0, 6.0 Hz, 2H), 7.71 (d, J=8.4 Hz, 2H), 7.58 (d, J=8.8 Hz, 1H), 7.55-7.52 (m, 3H), 7.34 (d, J=8.4 Hz, 2H), 3.72 (dd, J=7.6, 10.2 Hz, 1H), 3.53-3.44 (m, 1H), 3.43-3.36 (m, 1H), 3.21-3.15 (m, 1H), 2.28-2.16 (m, 1H), 1.98-1.90 (m, 1H), 1.42 (d, J=3.8 Hz, 9H).

Step 2. To a solution of tert-butyl 3-[4-[(3-nitro-6-phenyl-2-pyridyl)amino]phenyl]pyrrolidine-1-carboxylate (3.24 g, 7.04 mmol) in DMSO (12.0 mL) and methanol (3.00 mL), Na₂S₂O₄ (3.68 g, 21.1 mmol) and 2-aminopyridine-3-carbaldehyde (1.03 g, 8.45 mmol) were added at 25° C. under N₂, and the mixture was stirred at 100° C. for 16 hrs. EtOAc (100 mL) and MeOH (50 mL) were added, the mixture was filtered, and the filtrate was concentrated under reduced pressure to provide the crude material. Water (100 mL) was added, and the mixture was extracted with EtOAc (100 mL×3). The organic phase was washed with brine (100 mL), dried over Na₂SO₄, filtered, and concentrated under reduced pressure. The crude was purified by flash chromatography eluting with PE/EtOAc (from 0%-40% of EtOAc in PE) to give tert-butyl 3-[4-[2-(2-amino-3-pyridyl)-5-phenyl-imidazo[4,5-b]pyridin-3-yl]phenyl]pyrrolidine-1-carboxylate (246 mg, 8.08% yield) as a yellow solid. LCMS Rₜ=0.944 min in 1.5 min chromatography, purity 94.460%, MS ESI calcd. for 532.26 [M+H]⁺ 533.26, found 477.1.

Step 3. To a solution of tert-butyl 3-[4-[2-(2-amino-3-pyridyl)-5-phenyl-imidazo[4,5-b]pyridin-3-yl]phenyl]pyrrolidine-1-carboxylate (720 mg, 1.35 mmol) in DCM (5.00 mL), TFA (5.00 mL, 1.35 mmol) was added at 25° C., and the mixture was stirred at 25° C. for 16 hrs. The mixture was concentrated under reduced pressure to give 3-[5-phenyl-3-(4-pyrrolidin-3-ylphenyl)imidazo[4,5-b]pyridin-2-yl]pyridin-2-amine (1.30 g, crude) as a green oil. The crude was used directly in the next step without purification. ¹H NMR (400 MHz, DMSO-d₆) δ=8.35 (d, J=8.4 Hz, 1H), 8.12 (dd, J=1.4, 6.2 Hz, 1H), 8.08-8.02 (m, 3H), 7.87 (dd, J=1.6, 7.6 Hz, 1H), 7.61-7.54 (m, 4H), 7.51-7.46 (m, 2H), 7.45-7.39 (m, 1H), 6.88 (dd, J=6.4, 7.4 Hz, 1H), 6.06-6.01 (m, 2H), 3.77-3.67 (m, 1H), 3.62-3.53 (m, 1H), 3.51-3.41 (m, 1H), 3.34-3.24 (m, IH), 3.22-3.12 (m, 1H), 2.48-2.40 (m, 1H), 2.06-1.97 (m, 1H).

Step 4. To a solution of 3-[5-phenyl-3-(4-pyrrolidin-3-ylphenyl)imidazo[4,5-b]pyridin-2-yl]pyridin-2-amine (216 mg, 0.499 mmol) in DCM (6.00 mL), TEA (3.00 mL, 1.50 mmol) (pH ~7), and methyl 4-formylcyclohexanecarboxylate (255 mg, 1.50 mmol) were added and the mixture was stirred at 25° C. for 2 hrs. NaBH(OAc)₃ (529 mg, 2.50 mmol) was added and the mixture was stirred at 25° C. for 16 hrs. Water (20 mL) was added and the mixture was extracted with EtOAc (20 mL×3). The organic phase was washed with brine (20 mL), dried over Na₂SO₄, filtered, and concentrated under reduced pressure to give a crude. The crude was purified by flash chromatography eluting with EtOAc/MeOH (from 0%-30% of MeOH in EtOAc) to give methyl 4-[[3-[4-[2-(2-amino-3-pyridyl)-5-phenyl-imidazo[4,5-b]pyridin-3-yl]phenyl]pyrrolidin-1-yl]methyl]cyclohexanecarboxylate (160 mg, 54.6% yield) as a yellow oil. ¹H NMR (400 MHz, DMSO-d₆) δ=8.26 (d, J=8.0 Hz, 1H), 8.05-7.96 (m, 4H), 7.52-7.35 (m, 8H), 7.19 (dd, J=1.6, 7.6 Hz, 1H), 6.95 (s, 2H), 6.41 (dd, J=4.8, 7.6 Hz, 1H), 1.91 (s, 3H), 1.89-1.82 (m, 4H), 1.78-1.65 (m, 3H), 1.61-1.43 (m, 3H), 1.39-1.22 (m, 4H), 1.06-0.86 (m, 4H).

Step 5. To a solution of methyl 4-[[3-[4-[2-(2-amino-3-pyridyl)-5-phenyl-imidazo[4,5-b]pyridin-3-yl]phenyl]pyrrolidin-1-yl]methyl]cyclohexanecarboxylate (140 mg, 0.239 mmol) in THF (1.00 mL) and water (1.00 mL), LiOH·H₂O (25.0 mg, 0.597 mmol) was added at 25° C., and the mixture was stirred for 16 hrs. The mixture was extracted with EtOAc (10 mL) and HCl (3 mL) was added to the water phase. The water phase was purified by prep-HPLC (Xtimate C18 150*40 mm*5 um water(FA)-ACN) to give 4-[[3-[4-[2-(2-amino-3-pyridyl)-5-phenyl-imidazo[4,5-b]pyridin-3-yl]phenyl]pyrrolidin-1-yl]methyl]cyclohexanecarboxylic acid I-78 (24.0 mg, 17.3% yield) as a yellow solid. ¹H NMR (400 MHz, DMSO-d₆) δ=8.26 (d, J=8.8 Hz, 1H), 8.18 (s, 1H), 8.03-7.95 (m, 4H), 7.49-7.44 (m, 4H), 7.43-7.37 (m, 3H), 7.18 (dd, J=1.8, 7.8 Hz, 1H), 6.41 (dd, J=4.8, 7.6 Hz, 1H), 2.98 (t, J=8.8 Hz, 1H), 2.75-2.66 (m, 2H), 2.56-2.51 (m, 2H), 2.38-2.27 (m, 3H), 2.17-2.08 (m, 1H), 1.85 (q, J=12.4 Hz, 5H), 1.50-1.40 (m, 1H), 1.34-1.23 (m, 2H), 0.96-0.85 (m, 2H). HPLC Rₜ=2.968 min in 8.0 min chromatography, 10-80AB_8 min.1 cm, purity 100%. LCMS Rₜ=2.035 min in 4.0 min chromatography, purity 100.00%, MS ESI calcd. for 572.29 [M+H]⁺ 573.29, found 573.2.

### Example 71. Synthesis of 1-(1-(4-(2-(2-aminopyridin-3-yl)-5-phenyl-3H-imidazo[4,5-b]pyridin-3-yl)phenyl)ethyl)piperidine-4-carboxylic acid (Compound I-79)

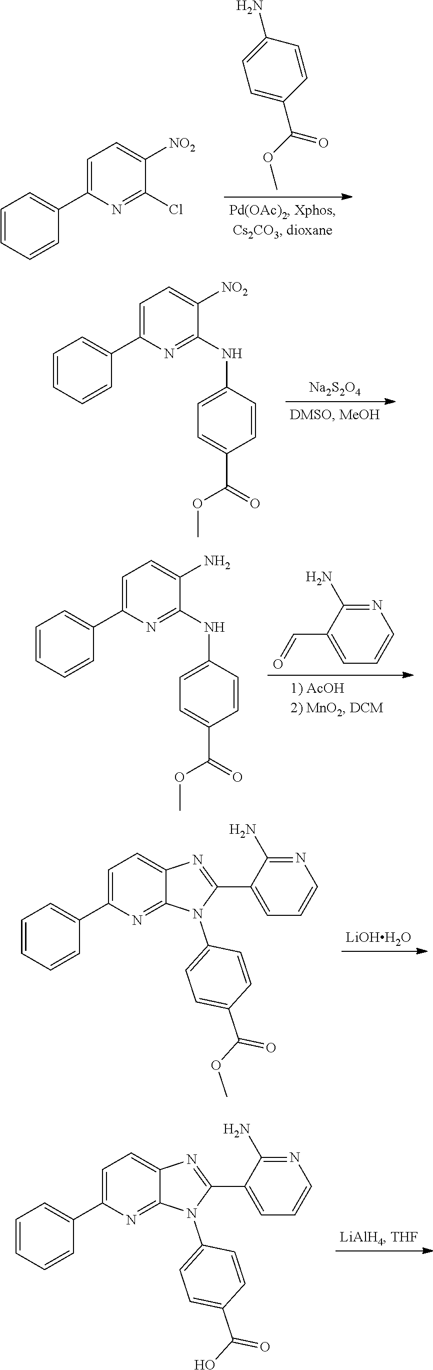

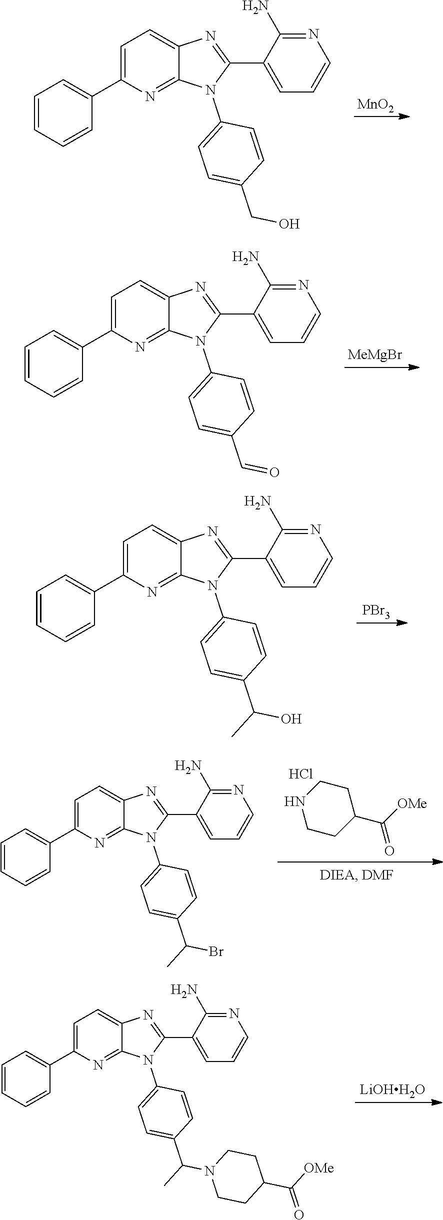

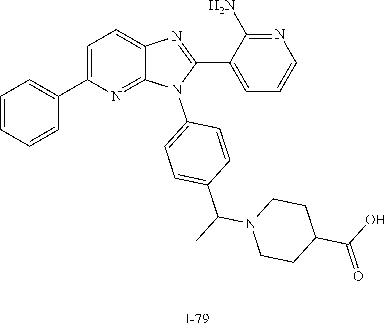

Step 1. To a solution of 2-chloro-3-nitro-6-phenyl-pyridine (10.0 g, 42.6 mmol) and methyl 4-aminobenzoate (6.44 g, 42.6 mmol) in 1,4-dioxane (150 mL), Cs₂CO₃ (41.7 g, 128 mmol), XPhos (4.06 g, 8.52 mmol) and Pd(OAc)₂ (0.960 g, 4.26 mmol) were added under N₂. The reaction mixture was degassed and purged with N₂ (×3), and the mixture was stirred at 100° C. for 12 hr under N₂. Water (50 mL) was added to the reaction mixture, and the resulting mixture was extracted with EtOAc (50 mL×3). The combined organic phase was washed with brine (50 mL), dried over anhydrous Na₂SO₄, filtered and concentrated. The residue was purified by flash silica gel chromatography (eluent of 0-7% MeOH in DCM) to give methyl 4-((3-nitro-6-phenylpyridin-2-yl)amino)benzoate (11.6 g, 77.9% yield) as a yellow solid. ¹H NMR (DMSO-d₆, 400 MHz) δ_{H}=10.24 (s, 1H), 8.64 (d, J=8.8 Hz, 1H), 8.17-8.10 (m, 2H), 8.04-7.92 (m, 4H), 7.70 (d, J=8.8 Hz, 1H), 7.59-7.54 (m, 3H), 3.86 (s, 3H).

Step 2. To a solution of methyl 4-((3-nitro-6-phenylpyridin-2-yl)amino)benzoate (11.0 g, 31.5 mmol) in DMSO (50.0 mL) and methanol (50.0 mL), Na₂S₂O₄ (11.0 g, 63.0 mmol) was added under N₂, and the mixture was stirred at 100° C. for 12 hrs. The reaction mixture was concentrated. aq. LiCl (300 mL) was added to the residue, and the resulting mixture was extracted with EtOAc (300 mL×3). The combined organic phase was washed with brine (300 mL), dried over anhydrous Na₂SO₄, filtered and concentrated to give methyl 4-((3-amino-6-phenylpyridin-2-yl)amino)benzoate (8.20 g, crude) as a light yellow oil, which was used directly in the next step. ¹H NMR (DMSO-d₆, 400 MHz) δ_{H}=8.39 (s, 1H), 7.96-7.91 (m, 2H), 7.91-7.88 (m, 2H), 7.84-7.81 (m, 2H), 7.44-7.39 (m, 2H), 7.37 (d, J=8.0 Hz, 1H), 7.30-7.24 (m, 1H), 7.06 (d, J=8.0 Hz, 1H), 5.37 (s, 2H), 3.81 (s, 3H).

Step 3. To a solution of methyl 4-((3-amino-6-phenylpyridin-2-yl)amino)benzoate (8.00 g, 25.1 mmol) in acetic acid (50.0 mL), 2-aminopyridine-3-carbaldehyde (4.13 g, 33.8 mmol) was added and the mixture was stirred at 110° C. for 1 hr. The reaction mixture was concentrated directly. DCM (50 mL) and MnO₂ (10.89 g, 125 mmol) were added into the residue and the mixture was stirred at 50° C. for 16 hrs. The reaction mixture was filtered, and the filtrate was concentrated to dryness. The residue was purified by flash silica gel chromatography (eluent of 0-80% EtOAc in PE) to give methyl 4-(2-(2-aminopyridin-3-yl)-5-phenyl-3H-imidazo[4,5-b]pyridin-3-yl)benzoate (6.20 g, 58.7% yield) as a yellow oil. ¹H NMR (DMSO-d₆, 400 MHz) δ_{H}=8.29 (d, J=8.4 Hz, 1H), 8.15-8.00 (m, 6H), 7.67 (d, J=8.4 Hz, 2H), 7.50-7.37 (m, 3H), 7.26-7.21 (m, 1H), 6.89 (s, 2H), 6.46 (dd, J=4.8, 7.6 Hz, 1H), 3.90 (s, 3H).

Step 4. To a solution of methyl 4-(2-(2-aminopyridin-3-yl)-5-phenyl-3H-imidazo[4,5-b]pyridin-3-yl)benzoate (4.00 g, 9.49 mmol) in water (20.0 mL) and THF (20.0 mL), LiOH·H₂O (1.99 g, 47.5 mmol) was added and the mixture was stirred at 25° C. for 12 hrs. The reaction mixture was concentrated directly. The residue was purified by prep-HPLC (Column: Welch Xtimate C18 150*30 mm*5 μm; mobile phase: [water (HCl)-ACN]; B %: 0%-35%: 25 min) to give 4-(2-(2-aminopyridin-3-yl)-5-phenyl-3H-imidazo[4,5-b]pyridin-3-yl)benzoic acid (2.10 g, 54.3% yield) as a yellow solid. ¹H NMR (DMSO-d₆, 400 MHz) δ_{H}=8.56-8.05 (m, 9H), 7.92-7.88 (m, 1H), 7.72 (d, J=8.4 Hz, 2H), 7.52-7.40 (m, 3H), 6.94-6.89 (m, 1H).

Step 5. To a solution of 4-(2-(2-aminopyridin-3-yl)-5-phenyl-3H-imidazo[4,5-b]pyridin-3-yl)benzoic acid (1.50 g, 3.56 mmol) in THF (20 mL), LiAlH₄ (6.04 g, 14.2 mmol) was added at 0° C., and the reaction mixture was stirred at 25° C. for 2 hrs. The reaction was quenched with saturated aqueous NH₄Cl (20 mL) dropwise. The resulting mixture was extracted with EtOAc (20 mL×3), and the combined organic phase was dried over anhydrous Na₂SO₄, filtered and concentrated to give [4-[2-(2-amino-3-pyridyl)-5-phenyl-imidazo[4,5-b]pyridin-3-yl]phenyl]methanol (1.30 g, 92.4% yield) as a yellow solid, which was used in the next step without further purification.

Step 6. To a solution of [4-[2-(2-amino-3-pyridyl)-5-phenyl-imidazo[4,5-b]pyridin-3-yl]phenyl]methanol (1.30 g, 3.30 mmol) in DCE (6.0.00 mL), MnO₂ (2.87 g, 33.0 mmol) was added and the reaction mixture was stirred at 60° C. for 12 hrs. The mixture was filtered via a celite pad and the filter cake was washed with DCM/EtOAc (1/1 (500 mL)). The organic phase was concentrated to give 4-[2-(2-amino-3-pyridyl)-5-phenyl-imidazo[4,5-b]pyridin-3-yl]benzaldehyde (1.20 g, 92.8% yield) as a yellow solid, which was used in the next step directly without further purification.

Step 7. To a solution of 4-[2-(2-amino-3-pyridyl)-5-phenyl-imidazo[4,5-b]pyridin-3-yl]benzaldehyde (520 mg, 1.33 mmol) in THF (6.00 mL), bromo(methyl)magnesium (792 mg, 6.64 mmol) was added at 0° C., and the reaction mixture was stirred at 25° C. for 1 hr. The reaction was quenched with saturated aqueous NH₄Cl (20 mL) dropwise. The resulting mixture was extracted with EtOAc (20 mL×3), and the combined organic phase was dried over anhydrous Na₂SO₄, filtered and concentrated. The residue was purified by flash silica gel chromatography (ISCO®; 12 g SepaFlash® Silica Flash Column, Eluent of 50% Ethyl acetate/Petroleum ether gradient @65 mL/min) to give 1-(4-(2-(2-aminopyridin-3-yl)-5-phenyl-3H-imidazo[4,5-b]pyridin-3-yl)phenyl)ethan-1-ol (189 mg, 34.9% yield) as a yellow solid. LCMS Rₜ=1.179 min in 2.5 min chromatography, purity 11.6%, MS EST calcd. for 407.17 [M+H]⁺ 408.17 found 408.1.

Step 8. To a solution of 1-[4-[2-(2-amino-3-pyridyl)-5-phenyl-imidazo[4,5-b]pyridin-3-yl]phenyl]ethanol (189 mg, 0.460 mmol) in DCM (2.00 mL), tribromophosphane (251 mg, 0.920 mmol) was added at 0° C., and the reaction mixture was stirred at 25° C. for 2 hrs. The mixture was diluted with saturated aqueous NaHCO₃ (10 mL) and extracted with DCM (10 mL×3). The organic layer was dried over Na₂SO₄, filtered and concentrated to give 3-[3-[4-(1-bromoethyl)phenyl]-5-phenyl-imidazo[4,5-b]pyridin-2-yl]pyridin-2-amine (203 mg, 0.430 mmol) as a yellow solid, which was used in the next step without further purification.

Step 9. To a solution of 3-[3-[4-(0-bromoethyl)phenyl]-5-phenyl-imidazo[4,5-b]pyridin-2-yl]pyridin-2-amine (203 mg, 0.430 mmol) and methyl piperidine-4-carboxylate (124 mg, 0.860 mmol) in DMF (2.00 mL), DIEA (167 mg, 1.29 mmol) was added and the reaction mixture was stirred at 25° C. for 0.5 hr. The mixture was diluted with water (30 mL), extracted with EtOAc (20 mL×3), washed with brine (30 mL), and the organic layer was dried over Na₂SO₄, filtered and concentrated. The crude product was purified by prep-HPLC (column: Welch Xtimate C18 40*200 mm 7 μm; mobile phase: water (NH₄HCO₃)-MeOH; B %: 60%-100%, 25 min) to give methyl 1-[1-[4-[2-(2-amino-3-pyridyl)-5-phenyl-imidazo[4,5-b]pyridin-3-yl]phenyl]ethyl]piperidine-4-carboxylate (50.0 mg, 21.9% yield) as a light yellow solid. LCMS Rₜ=1.956 min in 4 min chromatography, purity 97.0%, MS ESI calcd. for 532.26 [M+H]⁺ 533.26, found 533.3.

Step 10. To a solution of methyl 1-[1-[4-[2-(2-amino-3-pyridyl)-5-phenyl-imidazo[4,5-b]pyridin-3-yl]phenyl]ethyl]piperidine-4-carboxylate (50.0 mg, 0.0900 mmol) in THF (1.00 mL), LiOH·H₂O (12.0 mg, 0.280 mmol) and water (1.00 mL) were added and the reaction mixture was stirred at 25° C. for 2 hrs. The reaction mixture was concentrated, and the residue was diluted with water (10 mL) and extracted with PE (10 mL×3). The pH of the aqueous phase was adjusted to ~5 with HCl (2 M). The resulting mixture was extracted with DCM (10 mL×3). The combined organic phase was dried over anhydrous Na₂SO₄, filtered and concentrated. The crude product was purified by prep-HPLC (column: Welch Xtimate C18 40*200 mm 7 μm; mobile phase: [water (FA)-ACN]; B %: 0%-34%, 25 min) to give 1-(1-(4-(2-(2-aminopyridin-3-yl)-5-phenyl-3H-imidazo[4,5-b]pyridin-3-yl)phenyl)ethyl)piperidine-4-carboxylic acid I-79 (34.5 mg, 70.3% yield) as a light yellow solid. ¹H NMR (DMSO-d₆, 400 MHz) δ=8.25 (d, J=8.4 Hz, 1H), 8.19-8.14 (m, 1H), 8.04-7.93 (m, 4H), 7.54-7.35 (m, 7H), 7.15-7.00 (m, 3H), 6.39-6.32 (m, 1H), 3.77-3.76 (m, 1H), 2.94 (d, J=9.6 Hz, 1H), 2.77 (d, J=10.8 Hz, 1H), 2.19-1.83 (m, 5H), 1.69-1.50 (m, 2H), 1.39 (d, J=6.4 Hz, 3H). HPLC Rₜ=3.012 min in 8 min chromatography, purity 100%. LCMS Rₜ=1.904 min in 4 min chromatography, purity 96.0/0, MS ESI calcd. for 518.24 [M+H]⁺ 519.24, found 519.2.

### Example 71. Synthesis of 2-(4-(2-(2-aminopyridin-3-yl)-5-phenyl-3H-imidazo[4,5-b]pyridin-3-yl)benzoyl)-2-azaspiro[3.3]heptane-6-carboxylic acid (Compound I-80)

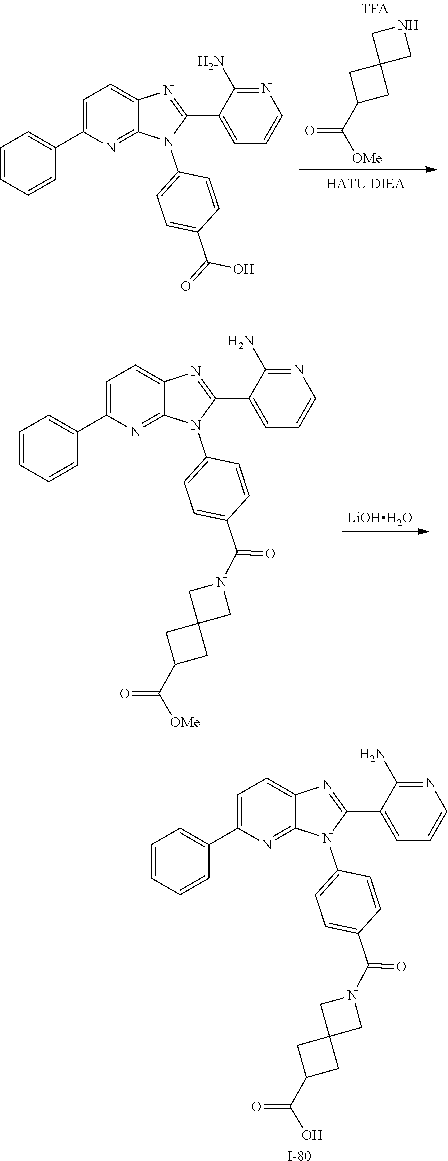

Step 1. To a solution of 4-[2-(2-amino-3-pyridyl)-5-phenyl-imidazo[4,5-b]pyridin-3-yl]benzoic acid (100 mg, 0.245 mmol) in DCM (2.00 mL), DIEA (0.120 mL, 0.736 mmol) and HATU (373 mg, 0.982 mmol) were added and the mixture was stirred at 25° C. for 30 mins. To the mixture a solution of methyl 2-azaspiro[3.3]heptane-6-carboxylate TFA (62.0 mg, 0.245 mmol) and DIEA (0.120 mL, 0.736 mmol) in DCM (2.00 mL) were added and the mixture was stirred at 25° C. for 12 hrs. Water (5 mL) was added to the reaction mixture and the resulting mixture was extracted with EtOAc (10 mL×3). The combined organic phase was washed with brine (10 mL), dried over anhydrous Na₂SO₄, filtered and concentrated. The crude was purified by flash column (0-80% of EtOAc in PE) to give methyl 2-[4-[2-(2-amino-3-pyridyl)-5-phenyl-imidazo[4,5-b]pyridin-3-yl]benzoyl]-2-azaspiro[3.3]heptane-6-carboxylate (100 mg, 74.8% yield) as a yellow oil. LCMS Rₜ=0.851 min in 1.5 min chromatography, purity 64.799%, MS ESI calcd. For 544.22 [M+H]⁺ 545.22, found 545.4.

Step 2. To a solution of methyl 2-[4-[2-(2-amino-3-pyridyl)-5-phenyl-imidazo[4,5-b]pyridin-3-yl]benzoyl]-2-azaspiro[3.3]heptane-6-carboxylate (100 mg, 0.184 mmol) in THF (2.00 mL) and water (2.00 mL), LiOH·H₂O (15.0 mg, 0.367 mmol) and one drop of methanol (0.1 mL) were added and the mixture stirred at 30° C. for 1 hr. The crude was purified by prep-HPLC (column: Xtimate C18 150*40 mm*5 μm; mobile phase:water(HCl)-ACN: B %: 20%-60%, 30 min) to give 2-[4-[2-(2-amino-3-pyridyl)-5-phenyl-imidazo[4,5-b]pyridin-3-yl]benzoyl]-2-azaspiro[3.3]heptane-6-carboxylic acid 1-80 (14.0 mg, 14.4% yield) as a yellow solid. LCMS Rₜ=3.011 min in 4 min chromatography, purity 100%, MS ESI calcd. For 530.21 [M+H]⁺ 531.21, found 532.3. HPLC Rₜ=4.133 min in 8 min chromatography, purity 100%. ¹H NMR (400 MHz, DMSO-d₆) δ=8.35 (d, J=8.4 Hz, 1H), 8.08-8.00 (m, 5H), 7.86 (dd, J=1.6, 7.6 Hz, 1H), 7.82-7.76 (m, 2H), 7.63 (d, J=8.4 Hz, 2H), 7.50-7.45 (m, 2H), 7.44-7.38 (m, 1H), 6.92-6.86 (m, 1H), 4.42-4.27 (m, 2H), 4.13-3.98 (m, 2H), 3.00-2.85 (m, 1H), 2.46-2.39 (m, 2H), 2.36-2.28 (m, 2H).

### Example 72. Synthesis of 3-((1-(4-(2-(2-aminopyridin-3-yl)-5-phenyl-3H-imidazo[4,5-b]pyridin-3-yl)-2-fluorophenyl)ethyl)amino)cyclopentane-1-carboxylic acid (Compound I-81)

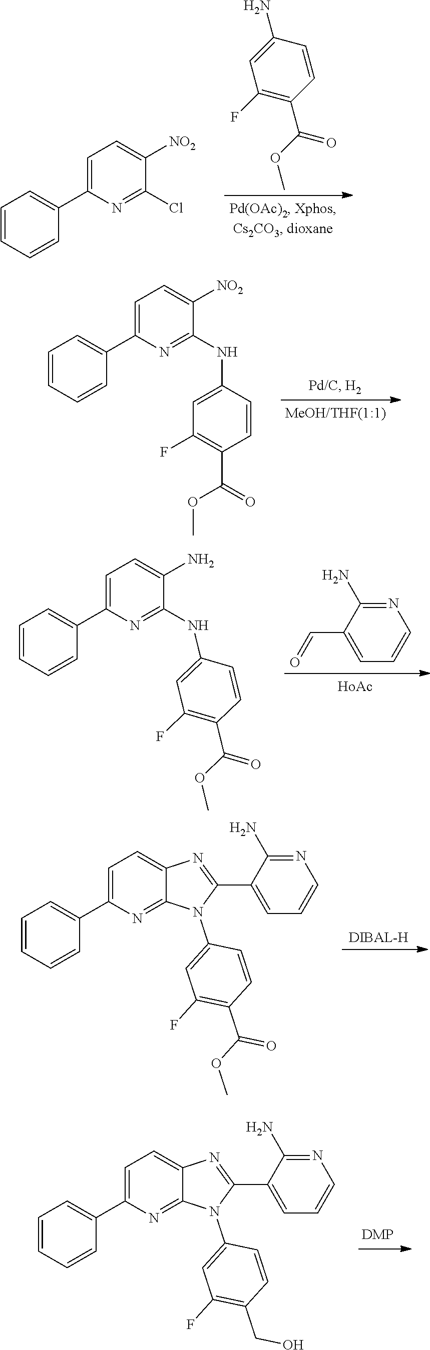

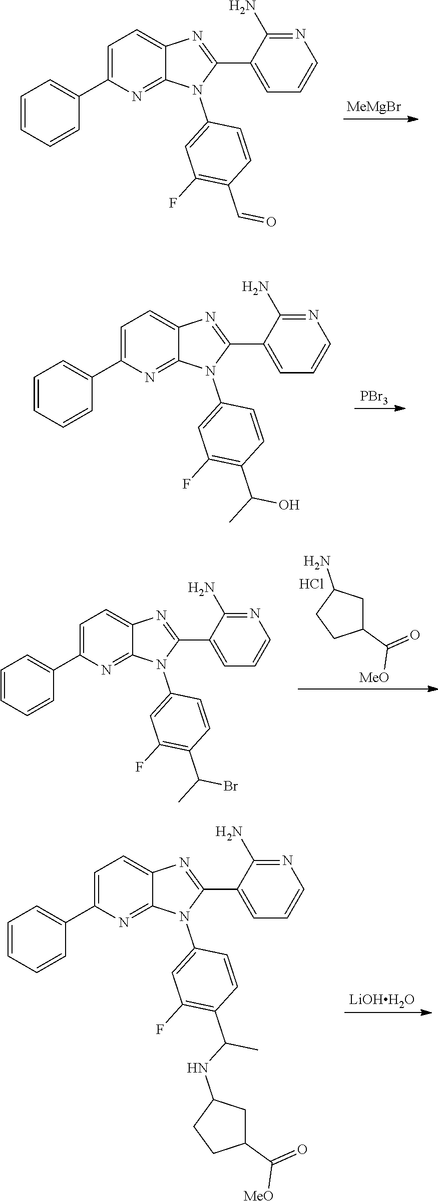

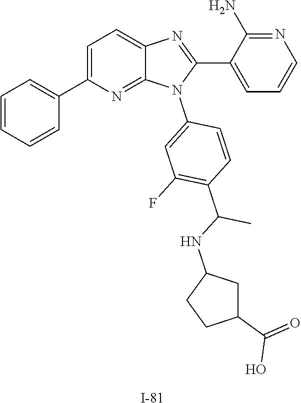

Step 1. To a solution of 2-chloro-3-nitro-6-phenylpyridine (6.00 g, 25.6 mmol) and methyl 4-amino-2-fluorobenzoate (4.33 g, 25.6 mmol) in 1,4-dioxane (100 mL), XPhos (610 mg, 1.28 mmol), Pd(OAc)₂ (1.01 g, 1.28 mmol) and Cs₂CO₃ (24.99 g, 76.7 mmol) were added at 25° C., and the mixture was stirred at 110° C. for 5 hrs. The reaction mixture was filtered and the filter cake was washed with DCM (300 mL). The filtrate was concentrated and the residue was dissolved in water (100 mL). The resulting suspension was extracted with DCM (200 mL×3). The combined organic phase was washed with brine (100 mL), dried over anhydrous Na₂SO₄, filtered and concentrated. The residue was purified by Combi-Flash column (EtOAc in PE=0%-15%) to afford methyl 2-fluoro-4-((3-nitro-6-phenylpyridin-2-yl)amino)benzoate (8.64 g, 74.4% yield) as a yellow solid. ¹H NMR (DMSO-d₆, 400 MHz) δ_{H}=10.26 (s, 1H), 8.63 (d, J=8.8 Hz, 1H), 8.11 (dd, J=2.8, 6.8 Hz, 2H), 7.98-7.85 (m, 2H), 7.77-7.65 (m, 2H), 7.61-7.51 (m, 3H), 3.85 (s, 3H).

Step 2. To a solution of methyl 2-fluoro-4-((3-nitro-6-phenylpyridin-2-yl)amino)benzoate (8.30 g, 22.6 mmol) in THF (50.0 mL) and MeOH (50.0 mL), Pd/C (4.00 g, 10% purity) was added under an Ar atmosphere. The suspension was degassed and purged with H₂ (×3), and the reaction mixture was stirred under H₂ (15 psi) at 25° C. for 12 hrs. The reaction mixture was filtered and the filter cake was washed with MeOH/DCM (1:1, 1 L). The resulting suspension was concentrated directly to afford methyl 4-((3-amino-6-phenylpyridin-2-yl)amino)-2-fluorobenzoate (7.75 g, crude) as a yellow solid. ¹H NMR (DMSO-d₆, 400 MHz) δ_{H}=8.71 (s, IH), 7.96-7.86 (m, 3H), 7.82 (t, J=8.8 Hz, 1H), 7.50-7.37 (m, 4H), 7.32-7.23 (m, 1H), 7.09 (d, J=8.0 Hz, 1H), 5.44 (s, 2H), 3.80 (s, 3H).

Step 3. To a solution of methyl 4-((3-amino-6-phenylpyridin-2-yl)amino)-2-fluorobenzoate (7.75 g, 23.0 mmol) in acetic acid (50.0 mL), 2-aminonicotinaldehyde (3.37 g, 27.6 mmol) was added at 25° C., and the mixture was stirred at 100° C. for 2 hrs. EtOAc (50 mL) was added and the resulting mixture was stirred at 25° C. for 1 hr. The mixture was filtered and the filter cake was washed with EtOAc (50 mL). The filtrate was concentrated directly. The filter cake was dissolved in DCM (100 mL). The resulting suspension was washed with saturated aqueous NaHCO₃ (30 mL×3). The combined organic phase was washed with brine (50 mL), dried over anhydrous Na₂SO₄, filtered and concentrated. The filtrate was purified by Combi-Flash column (EtOAc in PE=0%-58%) to afford methyl 4-(2-(2-aminopyridin-3-yl)-5-phenyl-3H-imidazo[4,5-b]pyridin-3-yl)-2-fluorobenzoate (500 mg, 4.95% yield) as a yellow solid. ¹H NMR (DMSO-d₆, 400 MHz,) δ_{H}=8.29 (d, J=8.4 Hz, 1H), 8.13-7.95 (m, 5H), 7.65 (d, J=11.6 Hz, 1H), 7.53-7.38 (m, 4H), 7.34 (d, J=7.2 Hz, 1H), 6.78 (s, 2H), 6.52 (dd, J=4.8, 6.8 Hz, 1H), 3.89 (s, 3H).

Step 4. To a solution of methyl 4-(2-(2-aminopyridin-3-yl)-5-phenyl-3H-imidazo[4,5-b]pyridin-3-yl)-2-fluorobenzoate (500 mg, 1.14 mmol) in DCM (8.00 mL), DIBAL-H (5.70 mL, 5.69 mmol) was added at 0° C. under N₂ atmosphere, and the mixture was stirred at 25° C. for 4 hrs. The reaction was quenched with saturated aqueous potassium sodium tartrate (15 mL) dropwise at 0° C. and the resulting mixture was extracted with DCM (15 mL×3). The combined organic phase was washed with brine (8 mL), dried over anhydrous Na₂SO₄, filtered and concentrated. The residue was purified by Combi-Flash column (EtOAc in PE, 0%-70%) to afford (4-(2-(2-aminopyridin-3-yl)-5-phenyl-3H-imidazo[4,5-b]pyridin-3-yl)-2-fluorophenyl)methanol (255 mg, 54.5% yield) as a yellow solid. ¹H NMR (DMSO-d₆, 400 MHz,) δ_{H}=8.27 (d, J=8.4 Hz, 1H), 8.07-7.98 (m, 4H), 7.61 (t, J=8.0 Hz, 1H), 7.50-7.44 (m, 3H), 7.41 (d, J=7.2 Hz, 1H), 7.30 (d, J=8.0 Hz, 2H), 6.88 (s, 2H), 6.48 (dd, J=4.8, 7.6 Hz, 1H), 4.63 (d, J=6.0 Hz, 2H).

Step 5. To a solution of (4-(2-(2-aminopyridin-3-yl)-5-phenyl-3H-imidazo[4,5-b]pyridin-3-yl)-2-fluorophenyl)methanol (255 mg, 0.620 mmol) in DCM (4.00 mL), DMP (315 mg, 0.740 mmol) was added at 0° C., and the mixture was stirred at 25° C. for 1 hr. The reaction was quenched with saturated aqueous NaHCO₃/Na₂S₂O₃ (6 mL, 1:1) drop-wise. The reaction mixture was extracted with DCM (8 mL×3) and the combined organic phase was washed with brine (8 mL), dried over anhydrous Na₂SO₄, filtered and concentrated. The residue was purified by Combi-Flash column (EtOAc in PE=0%-50%) to afford 4-(2-(2-aminopyridin-3-yl)-5-phenyl-3H-imidazo[4,5-b]pyridin-3-yl)-2-fluorobenzaldehyde (143 mg, 56.4% yield) as a yellow solid. ¹H NMR (DMSO-d₆, 400 MHz,) δ=10.25 (s, 1H), 8.31 (d, J=8.4 Hz, 1H), 8.09 (d, J=1.6 Hz, 1H), 8.08-8.05 (m, 2H), 8.04-8.01 (m, 1H), 8.01-7.98 (m, 1H), 7.71 (dd, J=1.6, 11.2 Hz, 1H), 7.53-7.46 (m, 3H), 7.45-7.40 (m, 1H), 7.36 (dd, J=2.0, 7.6 Hz, 1H), 6.77 (s, 2H), 6.52 (dd, J=4.8, 7.6 Hz, 1H).

Step 6. To a solution of 4-(2-(2-aminopyridin-3-yl)-5-phenyl-3H-imidazo[4,5-b]pyridin-3-yl)-2-fluorobenzaldehyde (400 mg, 0.980 mmol) in THF (5.00 mL), MeMgBr (3.30 mL, 9.77 mmol) was added at 0° C. under a N₂ atmosphere, and the mixture was stirred at 25° C. for 18 hrs. The reaction was quenched with aqueous NH₄Cl (10 mL), dropwise, and the resulting mixture was extracted with DCM (15 mL×3). The combined organic phase was washed with brine (5 mL), dried over anhydrous Na₂SO₄, filtered and concentrated. The residue was purified by Combi-Flash column (EtOAc in PE=0%-55%) to afford 1-(4-(2-(2-aminopyridin-3-yl)-5-phenyl-3H-imidazo[4,5-b]pyridin-3-yl)-2-fluorophenyl)ethan-1-ol (140 mg, 16.2% yield) as a yellow solid. LCMS Rₜ=1.225 min in 2.5 min chromatography, purity 48.38%, MS ESI calcd. For 425.17 [M+H]⁺ 426.17, found 426.1.

Step 7. To a solution of 1-(4-(2-(2-aminopyridin-3-yl)-5-phenyl-3H-imidazo[4,5-b]pyridin-3-yl)-2-fluorophenyl)ethan-1-ol (140 mg, 0.330 mmol) in DCM (5.00 mL), PBr₃ (0.0700 mL, 0.660 mmol) was added at 0° C. under N₂ atmosphere and the resulting mixture was stirred at 25° C. for 2 hrs under N₂ atmosphere. The reaction was quenched with saturated aqueous NaHCO₃ (5 mL), dropwise, and the resulting mixture was extracted with DCM (10 mL×3). The combined organic phase was washed with water (10 mL), dried over anhydrous Na₂SO₄, filtered and concentrated to afford 3-(3-(4-(1-bromoethyl)-3-fluorophenyl)-5-phenyl-3H-imidazo[4,5-b]pyridin-2-yl)pyridin-2-amine (100 mg, 62.2% yield) as a yellow solid. LCMS Rₜ=0.869 min in 1.5 min chromatography, purity 51.08%, MS ESI calcd for 487.08 [M+H]⁺ 489.08, found 490.0.

Step 8. To a solution of 3-(3-(4-(1-bromoethyl)-3-fluorophenyl)-5-phenyl-3H-imidazo[4,5-b]pyridin-2-yl)pyridin-2-amine (125 mg, 0.260 mmol) in DMF (1.00 mL), methyl 3-aminocyclopentane-1-carboxylate HCl (92.0 mg, 0.520 mmol), NaI (19.0 mg, 0.130 mmol) and DIEA (99.0 mg, 0.770 mmol) were added at 25° C., and the mixture was stirred at 80° C. for 12 hrs. The mixture was purified by prep-HPLC (column: Xtimate C18 200*40 mm*7 μm; mobile phase: [water(NH₄HCO₃)-MeOH]; B %:54%-94%,25 min) to afford methyl 3-((1-(4-(2-(2-aminopyridin-3-yl)-5-phenyl-3H-imidazo[4,5-b]pyridin-3-yl)-2-fluorophenyl)ethyl)amino)cyclopentane-1-carboxylate (30.0 mg, 21.3% yield) as a yellow solid. LCMS Rₜ=2.288 min in 2.5 min chromatography, purity 48.38%, MS ESI calcd. for 550.25 [M+H]⁺ 551.25, found 551.2.

Step 9. A solution of methyl 3-((1-(4-(2-(2-aminopyridin-3-yl)-5-phenyl-3H-imidazo[4,5-b]pyridin-3-yl)-2-fluorophenyl)ethyl)amino)cyclopentane-1-carboxylate (30.0 mg, 0.0600 mmol) and LiOH·H₂O (7.00 mg, 0.170 mmol) in THF (1.00 mL) and water (1.00 mL) was stirred at 25° C. for 1 hr. The mixture was purified by prep-HPLC (column: Xtimate C18 200*40 mm*7 μm; mobile phase: [water(HCl)-ACN]; B %: 0%-38%, 30 min) to afford 3-((1-(4-(2-(2-aminopyridin-3-yl)-5-phenyl-3H-imidazo[4,5-b]pyridin-3-yl)-2-fluorophenyl)ethyl)amino)cyclopentane-1-carboxylic acid 1-81 (5.50 ng, 20.9% yield) as a yellow solid. ¹H NMR (DMSO-d₆, 400 MHz,) δ1=8 30 (d, J=8.4 Hz, 1H), 8.05-7.86 (m, 4H), 7.84-7.68 (m, 2H), 7.58 (d, J=10.4 Hz, 1H), 7.51-7.32 (m, 4H), 6.90-6.72 (m, 1H), 4.74-4.59 (m, 1H), 3.45 (dt, J=8.0, 16.0 Hz, 1H), 2.98-2.71 (m, 1H), 2.34-2.11 (m, 1H), 2.10-1.71 (m, 4H), 1.68-1.46 (m, 4H). HPLC Rₜ=3.073 min in 8 min chromatography, purity 99.61%. LCMS Rₜ=1.973 min in 4 min chromatography, purity 100%, MS ESI calcd. for 536.23 [M+H]⁺ 537.23, found 537.2.

### Example 73. Synthesis of 2-(1-(4-(2-(2-aminopyridin-3-yl)-5-phenyl-3H-imidazo[4,5-b]pyridin-3-yl)benzoyl)pyrrolidin-3-yl)-2-methylpropanoic acid (Compound I-82)

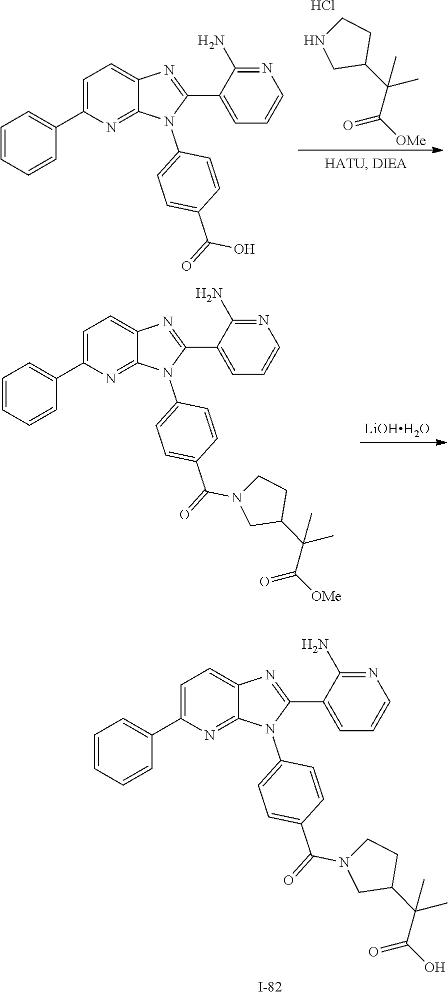

Step 1. To a solution of 4-[2-(2-amino-3-pyridyl)-5-phenyl-imidazo[4,5-b]pyridin-3-yl]benzoic acid (150 mg, 0.368 mmol) in DCM (2.00 mL), DIEA (0.180 mL, 1.10 mmol) and HATU (140 mg, 0.368 mmol) were added and the mixture was stirred at 25° C. for 30 mins. To the mixture, a solution of methyl 2-methyl-2-pyrrolidin-3-yl-propanoate (63.0 mg, 0.368 mmol), methyl 2-pyrrolidin-3-ylpropanoate (58.0 mg, 0.368 mmol) and DIEA (0.180 mL, 1.10 mmol) in DCM (1.00 mL) were added and the mixture was stirred at 25° C. for 12 hrs. HATU (40.0 mg) and methyl 2-methyl-2-pyrrolidin-3-yl-propanoate (40.0 mg) were added and the mixture was stirred at 25° C. for 2 hrs. Water (5 mL) was added to the reaction mixture, and the whole was extracted with EtOAc (10 mL×3). The combined organic phase was washed with brine (10 mL), dried over anhydrous Na₂SO₄, filtered and concentrated. The residue was purified by prep-HPLC (Xtimate C18 150*40 mm*5 μm, water (HCl)-ACN) to give methyl 2-[1-[4-[2-(2-amino-3-pyridyl)-5-phenyl-imidazo[4,5-b]pyridin-3-yl]benzoyl]pyrrolidin-3-yl]-2-methyl-propanoate (140 mg, 69.6% yield) as a yellow solid.

Step 2. To a solution of methyl 2-[1-[4-[2-(2-amino-3-pyridyl)-5-phenyl-imidazo[4,5-b]pyridin-3-yl]benzoyl]pyrrolidin-3-yl]-2-methyl-propanoate (100 mg, 0.178 mmol) in methanol (1.00 mL), THF (1.00 mL) and water (1.00 mL), LiOH·H₂O (37.0 mg, 0.892 mmol) was added and the mixture was stirred at 45° C. for 16 hrs. The mixture was concentrated. The residue was dissolved with DMSO and 1M HCl, and subsequently purified by prep-HPLC (Xtimate C18 150*40 mm*5 μm, water (HCl)-ACN) to give 2-(1-(4-(2-(2-aminopyridin-3-yl)-5-phenyl-3H-imidazo[4,5-b]pyridin-3-yl)benzoyl)pyrrolidin-3-yl)-2-methylpropanoic acid I-82 (22.0 mg, 21.2% yield) as a yellow solid. ¹H NMR (MeOD 400 MHz) δ_{H}=8.19 (d, J=8.3 Hz, 1H), 8.07-7.92 (m, 4H), 7.70 (d, J=8.0 Hz, 2H), 7.58 (d, J=8.3 Hz, 2H), 7.47-7.30 (m, 4H), 6.55-6.47 (m, 1H), 3.86-3.70 (m, 1H), 3.64-3.37 (m, 3H), 2.64-2.45 (m, 1H), 2.14-1.94 (m, 1H), 1.92-1.77 (m, 1H), 1.25 (d, J=5.0 Hz, 3H), 1.18 (d, J=10.3 Hz, 3H). HPLC Rₜ=4.416 min in 8 min chromatography, 0-60AB, purity 95.86%. LCMS Rₜ=3.205 min in 4 min chromatography, Xtimate C18 2.1*30 mm,3 um, purity 95.85%, MS ESI calcd. for 546.24 [M+H]⁺ 547.24, found 547.3.

### Example 74. Synthesis of 1-(1-(4-(2-(2-aminopyridin-3-yl)-5-phenyl-3H-imidazo[4,5-b]pyridin-3-yl)phenyl)ethyl)piperidine-3-carboxylic acid (Compound I-83)

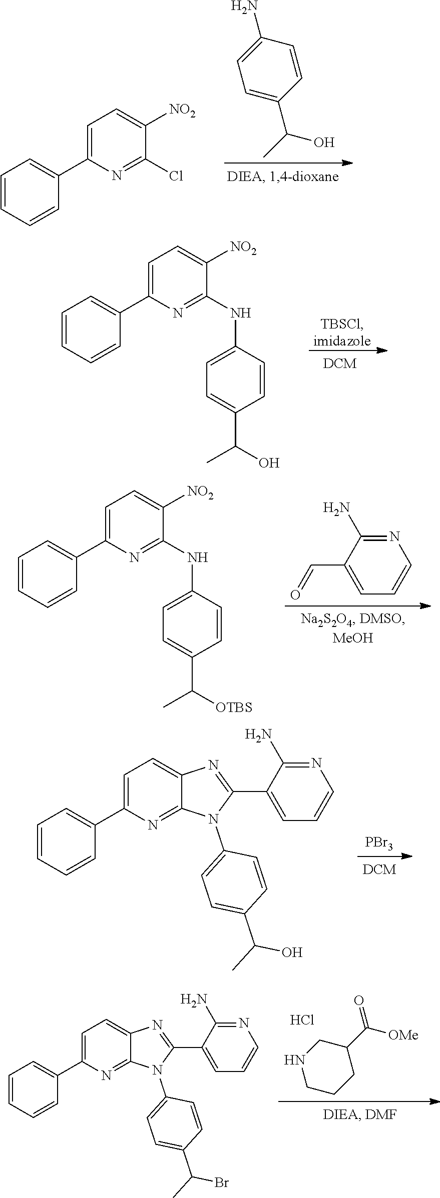

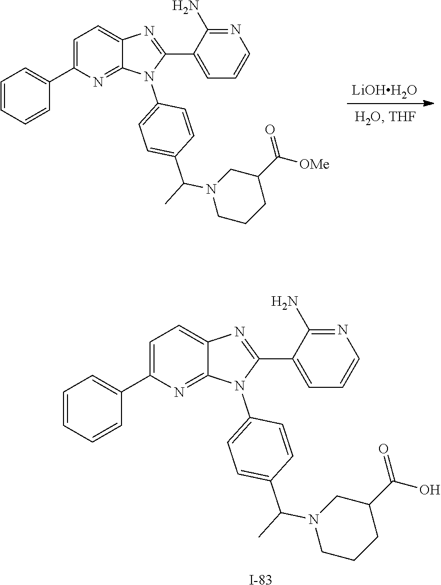

Step 1. To a solution of 2-chloro-3-nitro-6-phenyl-pyridine (5.00 g, 21.3 mmol) in 1,4-dioxane (50.0 mL), 1-(4-aminophenyl)ethanol (4.38 g, 32.0 mmol) and DIEA (11.0 mL, 63.9 mmol) were added at 25° C. and the reaction mixture was stirred at 80° C. for 12 hrs under N₂ atmosphere. The mixture was diluted with H₂O (100 mL) and extracted with EtOAc (100 mL×3). The organic layer was dried over Na₂SO₄ and concentrated. The residue was purified by flash silica gel chromatography (ISCO®, 120 g SepaFlash® Silica Flash Column, Eluent of 20% Ethyl acetate/Petroleum ether gradient @100 mL/min) to provide 1-(4-((3-nitro-6-phenylpyridin-2-yl)amino)phenyl)ethan-1-ol (5.00 g, 70.0% yield) as a yellow solid. LCMS Rₜ=0.931 min in 1.5 min chromatography, purity 54.84%, MS ESI calcd. for 335.13 [M+H]⁺ 336.13, found 336.0.

Step 2. To a solution of 1-[4-[(3-nitro-6-phenyl-2-pyridyl)amino]phenyl]ethanol (5.00 g, 14.9 mmol) in DCM (50.0 mL), imidazole (2.04 g, 29.8 mmol) and TBSCl (3.38 g, 22.4 mmol) were added at 25° C. and the reaction mixture was stirred for 12 hrs under N₂ atmosphere. The mixture was diluted with H₂O (50 mL) and extracted with DCM (40 mL×3). The organic layer was dried over Na₂SO₄ and concentrated. The residue was purified by flash silica gel chromatography (ISCO®; 80 g SepaFlash® & Silica Flash Column, Eluent of 3% ethyl acetate/petroleum ether gradient @100 mL/min) to provide N-[4-[1-[tert-butyl(dimethyl)silyl]oxyethyl]phenyl]-3-nitro-6-phenyl-pyridin-2-amine (6.80 g, crude) as a yellow liquid. ¹H NMR (DMSO-d₆, 400 MHz) δ=10.09 (s, 1H), 8.60 (d, J=8.6 Hz, 1H), 8.05-8.13 (m, 2H), 7.67 (d, J=8.5 Hz, 2H), 7.59 (d, J=8.8 Hz, 1H), 7.45-7.55 (m, 3H), 7.39 (d, J=8.4 Hz, 2H), 4.90-4.99 (m, 1H), 1.39 (d, J=6.4 Hz, 3H), 0.88 (s, 9H), 0.07 (s, 3H), −0.01 (s, 3H).

Step 3. To a solution of N-[4-[1-[tert-butyl(dimethyl)silyl]oxyethyl]phenyl]-3-nitro-6-phenyl-pyridin-2-amine (5.00 g, 14.9 mmol) and 2-aminopyridine-3-carbaldehyde (2.37 g, 19.4 mmol) in DMSO (40.0 mL) and methanol (10.0 mL), Na₂S₂O₄ (7.79 g, 44.7 mmol) was added at 25° C., and the resulting mixture was stirred at 100° C. for 2 hrs under N₂ atmosphere. The reaction was quenched with saturated aqueous NaHCO₃ (200 mL), dropwise, and the resulting mixture was extracted with EtOAc (100 mL×3). The combined organic phase was dried over anhydrous Na₂SO₄, filtered and concentrated. The residue was purified by flash silica gel chromatography (ISCO®; 80 g SepaFlash® Silica Flash Column, Eluent of 70% Ethyl acetate/Petroleum ether gradient @100 mL/min) to give 1-[4-[2-(2-amino-3-pyridyl)-5-phenyl-imidazo[4,5-b]pyridin-3-yl]phenyl]ethanol (1.29 g, 21.2% yield) as a yellow solid. LCMS Rₜ=1.156 min in 2.5 min chromatography, purity 34.7%, MS ESI calcd for 407.17 [M+H]⁺408.17, found 408.3.

Step 4. To a solution of 1-[4-[2-(2-amino-3-pyridyl)-5-phenyl-imidazo[4,5-b]pyridin-3-yl]phenyl]ethanol (1.29 g, 3.17 mmol) in DCM (20.0 mL), tribromophosphane (1.71 g, 6.33 mmol) was added at 0° C. and the resulting mixture was stirred at 25° C. for 2 hrs. The reaction was quenched with saturated aqueous NaHCO₃ (10 mL), dropwise, and the resulting mixture was extracted with DCM (30 mL×3). The combined organic phase was washed with water (20 mL), dried over anhydrous Na₂SO₄, filtered and concentrated. The residue was purified by flash silica gel chromatography (ISCO® 40 g SepaFlash® Silica Flash Column, Eluent of 60% Ethyl acetate/Petroleum ether gradient @80 mL/min) to give 3-[3-[4-(1-bromoethyl)phenyl]-5-phenyl-imidazo[4,5-b]pyridin-2-yl]pyridin-2-amine (311 mg, 20.9% yield) as a yellow solid. LCMS Rₜ=1.423 min in 2.5 min chromatography, purity 27.52%, MS ESI calcd. for 469.09, [M+H]⁺ 470.09, found 472.0.

Step 5. To a solution of 3-[3-[4-(1-bromoethyl)phenyl]-5-phenyl-imidazo[4,5-b]pyridin-2-yl]pyridin-2-amine (100 mg, 0.210 mmol) and methyl piperidine-3-carboxylate (50.0 mg, 0.270 mmol) in DMF (2.00 mL), DIEA (82.0 mg, 0.630 mmol) was added at 25° C. The reaction mixture was degassed and purged with N₂ (×3) and the mixture was stirred at 80° C. for 12 hrs under N₂ atmosphere. The mixture was diluted with H₂O (10 mL), extracted with EtOAc (10 mL×3), washed with brine (10 mL) and the organic layer was dried over Na₂SO₄ and concentrated to give methyl 1-[1-[4-[2-(2-amino-3-pyridyl)-5-phenyl-imidazo[4,5-b]pyridin-3-yl]phenyl]ethyl]piperidine-3-carboxylate (120 mg, crude) as a yellow solid, which was used in the next step directly without further purification. LCMS Rₜ=0.676 min in 1.5 min chromatography, purity 52.52%, MS ESI calcd. for 532.26 [M+H]⁺ 533.26, found 533.3.

Step 6. To a solution of methyl 1-[1-[4-[2-(2-amino-3-pyridyl)-5-phenyl-imidazo[4,5-b]pyridin-3-yl]phenyl]ethyl]piperidine-3-carboxylate (120 mg, 0.220 mmol) in THF (2.00 mL), LiOH·H₂O (28.0 mg, 0.670 mmol) and water (1.00 mL) were added and the reaction mixture was stirred at 25° C. for 2 hrs. The reaction mixture was concentrated, the residue was diluted with H₂O (10 mL) and extracted with PE (10 mL×3). The pH of the aqueous phase was adjusted to pH ~5 with HCl (2 M). The resulting mixture was extracted with DCM (10 mL×3). The combined organic phase was dried over anhydrous Na₂SO₄, filtered, and concentrated. The crude product was purified by prep-HPLC (column: Welch Xtimate C18 40*200 mm 7 μm; mobile phase: [water(FA)-ACN]; B %: 0%-34%, 25 min) to give 1-(1-(4-(2-(2-aminopyridin-3-yl)-5-phenyl-3H-imidazo[4,5-b]pyridin-3-yl)phenyl)ethyl)piperidine-3-carboxylic acid I-83 (25.7 mg, 22% yield) as a light yellow solid. ¹H NMR (DMSO-d₆, 400 MHz) δ=8.28 (d, J=8.4 Hz, 1H), 8.05-7.96 (m, 4H), 7.53-7.37 (m, 7H), 7.14 (d, J=9.6 Hz, 2H), 7.10-7.04 (m, 1H), 6.36-6.29 (m, IH), 3.76-3.61 (m, 1H), 2.88-2.66 (m, 2H), 2.46-2.32 (m, 1H), 2.04 (s, 2H), 1.86-1.746 (m, 1H), 1.72-1.59 (m, 1H), 1.38 (d, J=6.8 Hz, 5H) HPLC Rₜ=3.031 min in 8 min chromatography, purity 100.00%. LCMS Rₜ=1.912 min in 4 min chromatography, purity 100.00%, MS ESI calcd. for 518.24 [M+H]⁺ 519.24, found 519.2.

### Example 75. Synthesis of 1-(1-(4-(2-(2-aminopyridin-3-yl)-5-phenyl-3H-imidazo[4,5-b]pyridin-3-yl)phenyl)ethyl)pyrrolidine-3-carboxylic acid (Compound I-84)

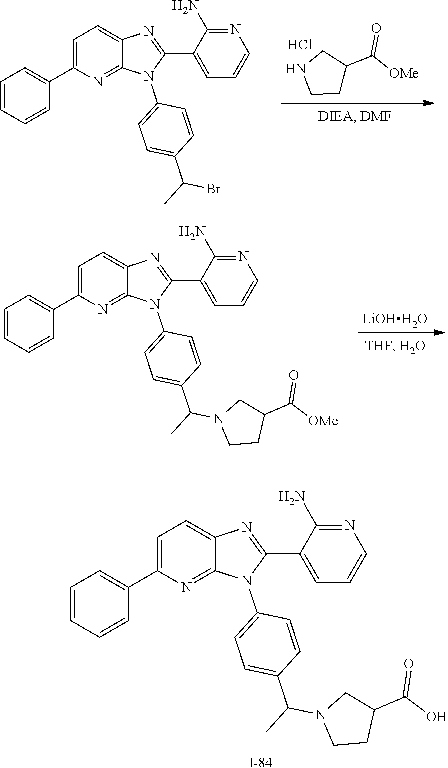

Step 1. To a solution of 3-[3-[4-(1-bromoethyl)phenyl]-5-phenyl-imidazo[4,5-b]pyridin-2-yl]pyridin-2-amine (100 mg, 0.210 mmol) and methyl pyrrolidine-3-carboxylate (46.0 mg, 0.270 mmol) in DMF (2.00 mL), DIEA (82.0 mg, 0.630 mmol) was added at 25° C. The reaction mixture was degassed and purged with N₂ (×3) and the mixture was stirred at 80° C. for 12 hrs under N₂ atmosphere. The mixture was diluted with H₂O (10 mL), extracted with EtOAc (10 mL×3), and washed with brine (10 mL). The organic layer was dried over Na₂SO₄ and concentrated to give methyl 1-[1-[4-[2-(2-amino-3-pyridyl)-5-phenyl-imidazo[4,5-b]pyridin-3-yl]phenyl]ethyl]pyrrolidine-3-carboxylate (130 mg, crude) as a yellow solid, which was used in the next step without further purification. LCMS Rₜ=1.162 min in 2.5 min chromatography, purity 43.55%, MS ESI calcd. for 518.24 [M+H]⁺ 519.24, found 519.2.

Step 2. To a solution of methyl 1-[1-[4-[2-(2-amino-3-pyridyl)-5-phenyl-imidazo[4,5-b]pyridin-3-yl]phenyl]ethyl]pyrrolidine-3-carboxylate (130 mg, 0.250 mmol) in THF (1.00 mL), LiOH·H₂O (310.0 mg, 0.730 mmol) and water (1.00 mL) were added and the reaction mixture was stirred at 25° C. for 2 hrs. The reaction mixture was concentrated, the residue was diluted with H₂O (10 mL) and extracted with PE (10 mL×3). The pH of the aqueous phase was adjusted to pH ~5 with HCl (2M). The resulting mixture was extracted with DCM (10 mL×3) and the combined organic phase was dried over anhydrous Na₂SO₄, filtered and concentrated. The crude product was purified by prep-HPLC (column: Welch Xtimate C18 150*25 mm*5 μm; mobile phase: [water(FA)-ACN]; B %: 0%-34%, 30 min) to give 1-(1-{4-(2-(2-aminopyridin-3-yl)-5-phenyl-3H-imidazo[4,5-b]pyridin-3-yl)phenyl}ethyl)pyrrolidine-3-carboxylic acid I-84 (33.9 mg, 26.8% yield) as a yellow solid. ¹H NMR (DMSO-d₆, 400 MHz) δ=8.28 (d, J=8.4 Hz, 1H), 8.15 (s, 1H), 8.06-7.96 (m, 4H), 7.53-7.37 (m, 7H), 7.13-7.06 (m, 3H), 6.38-6.32 (m, 1H), 3.38 (s, 1H), 2.98-2.66 (m, 3H), 2.62-2.56 (m, 1H), 2.37 (d, J=6.8 Hz, 1H), 2.01-1.92 (m, 2H), 1.38 (d, J=6.4 Hz, 3H). HPLC Rₜ=2.969 min in 8 min chromatography, purity 100%. LCMS Rₜ=1.867 min in 4 min chromatography, purity 99.46%, MS ESI calcd. for 504.23 [M+H]⁺ 505.23, found 505.2.

### Example 76. Synthesis of 1-[[4-[2-(2-amino-3-pyridyl)-5-phenyl-imidazo[4,5-b]pyridin-3-yl]phenyl]methyl]-3,3-dimethyl-piperidine-4-carboxylic acid (Compound I-85)

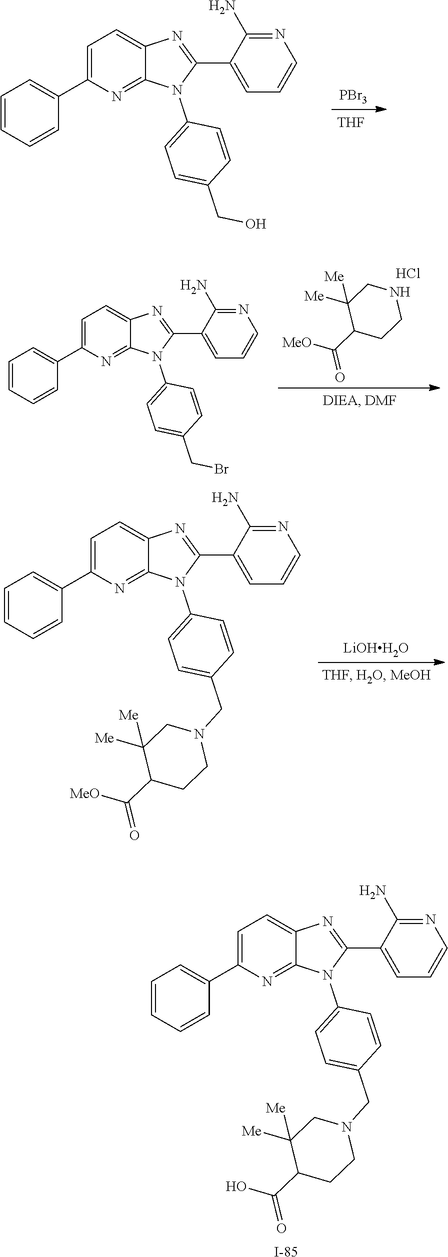

Step 1. To a solution of [4-[2-(2-amino-3-pyridyl)-5-phenyl-imidazo[4,5-b]pyridin-3-yl]phenyl]methanol (500 mg, 1.27 mmol) in THF (5.00 mL), PBr₃ (0.240 ml, 2.54 mmol) was added at 0° C. under N₂ atmosphere and the resulting mixture was stirred at 25° C. for 2 hrs under N₂ atmosphere. The reaction was quenched with saturated aqueous. NaHCO₃ (20 mL), dropwise, and the resulting mixture was extracted with DCM (20 mL×3). The combined organic phase was washed with water (20 mL), dried over anhydrous Na₂SO₄, filtered and concentrated to give 3-[3-[4-(bromomethyl)phenyl]-5-phenyl-imidazo[4,5-b]pyridin-2-yl]pyridin-2-anine (700 mg crude) as a yellow solid, which was directly used in the next step without further purification. LCMS Rₜ=3.512 min in 4 min chromatography, purity 77.9%, MS ESI calcd. For 455.07 [M+H]⁺ 456.07, found 457.1.

Step 2. To a solution of 3-[3-[4-(bromomethyl)phenyl]-5-phenyl-imidazo[4,5-b]pyridin-2-yl]pyridin-2-amine (200 mg, 0.438 mmol) in DMF (2.00 mL), methyl 3,3-dimethylpiperidine-4-carboxylate;hydrochloride (137 mg, 0.657 mmol) and DIEA (0.390 mL, 2.19 mmol) were added and the reaction stirred at 100° C. for 16 hrs. Water (10 mL) was added to the reaction mixture and the whole extracted with EtOAc (30 mL×3). The organic layer was dried over Na₂SO₄ and concentrated in vacuum. The crude material was purified by flash column (0-80% of EtOAc in PE) to give methyl 1-[[4-[2-(2-amino-3-pyridyl)-5-phenyl-imidazo[4,5-b]pyridin-3-yl]phenyl]methyl]-3,3-dimethyl-piperidine-4-carboxylate (40.0 mg, 16.7% yield) as a yellow oil. LCMS Rₜ=0.804 min in 1.5 min chromatography, purity 73.274%, MS ESI calcd. For 546.27 [M+H]⁺ 547.27, found 547.4.

Step 3. To a solution of methyl I-[[4-[2-(2-amino-3-pyridyl)-5-phenyl-imidazo[4,5-b]pyridin-3-yl]phenyl]methyl]-3,3-dimethyl-piperidine-4-carboxylate (50.0 mg, 0.0920 mmol) in THF (0.500 mL), water (0.500 mL) and methanol (1.00 mL), LiOH·H₂O (3.80 mg, 0.0920 mmol) was added and the reaction stirred at 50° C. for 16 hrs. The crude was purified by prep-HPLC (column: Welch Xtimate C18 150×30 mm×5 μm; mobile phase:water(FA)-ACN: B %: 20%-50%, 25 min) to give 1-[[4-[2-(2-amino-3-pyridyl)-5-phenyl-imidazo[4,5-b]pyridin-3-yl]phenyl]methyl]-3,3-dimethyl-piperidine-4-carboxylic acid 1-85 (12.0 mg, 23.5% yield) as a yellow solid. ¹H NMR (400 MHz, DMSO-d₆) δ=12.29-11.72 (m, 1H), 8.28 (d, J=8.4 Hz, 1H), 8.06-7.98 (m, 4H), 7.51-7.39 (m, 7H), 7.17 (d, J=6.4 Hz, 1H), 7.07 (s, 2H), 6.42-6.28 (m, 1H), 3.67-3.41 (m, 2H), 2.91 (d, J=8.4 Hz, 1H), 2.42-2.35 (m, 1H), 2.09-1.93 (m, 2H), 1.90-1.80 (m, 1H), 1.74 (d, J=10.8 Hz, 1H), 1.63 (d, J=10.0 Hz, 1H), 1.02-0.94 (m, 6H). HPLC Rₜ=3.739 min in 8 min chromatography, purity 99.636%. LCMS Rₜ=2.711 min in 4 min chromatography, purity 100%, MS ESI calcd. For 532.26 [M+H]⁺ 533.26, found 533.1.

### Example 77. Synthesis of 1-[[4-[2-(2-amino-3-pyridyl)-5-phenyl-imidazo[4,5-b]pyridin-3-yl]phenyl]methyl]-4-methyl-piperidine-4-carboxylic acid (Compound I-86)

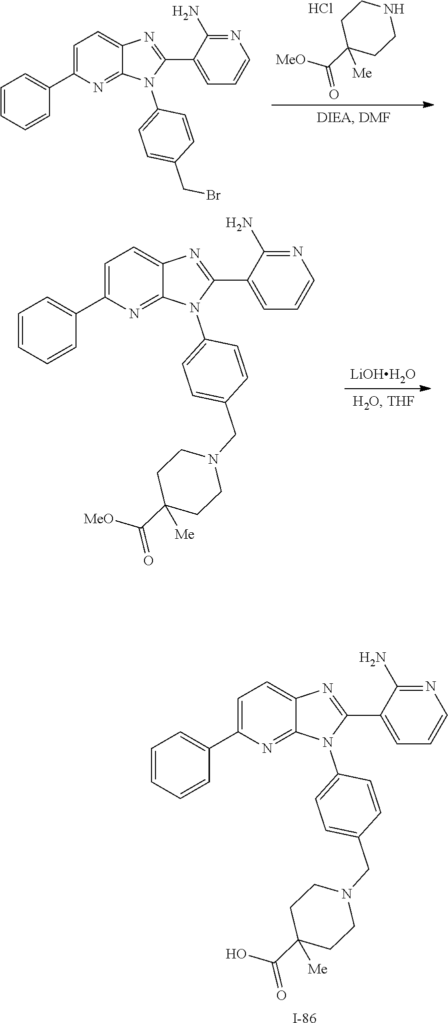

Step 1. To a solution of 3-[3-[4-(bromomethyl)phenyl]-5-phenyl-imidazo[4,5-b]pyridin-2-yl]pyridin-2-amine (200 mg, 0.438 mmol) in DMF (2 mL), methyl 4-methylpiperidine-4-carboxylate HCl (118 mg, 0.66 mmol) and DIEA(283 mg, 2.19 mmol) were added and the reaction was stirred at 100° C. for 16 hrs Water (10 mL) was added to the reaction mixture and the whole extracted with EtOAc (30 mL×3). The organic layer was dried over Na₂SO₄ and concentrated in vacuum to give methyl 1-[[4-[2-(2-amino-3-pyridyl)-5-phenyl-imidazo[4,5-b]pyridin-3-yl]phenyl]methyl]-4-methyl-piperidine-4-carboxylate (60.0 mg, 25.7% yield). LCMS Rₜ=0.788 min in 1.5 min chromatography, purity 66.941%, MS ESI calcd. For 532.26 [M+H]⁺ 533.26, found 533.4.

Step 2. To a solution of methyl 1-[[4-[2-(2-amino-3-pyridyl)-5-phenyl-imidazo[4,5-b]pyridin-3-yl]phenyl]methyl]-4-methyl-piperidine-4-carboxylate (60.0 mg, 0.11 mmol) in THF (1 mL) and water (1 mL), LiOH·H₂O (4.19 mg, 0.451 mmol) was added and the reaction was stirred at 50° C. for 16 hrs. The mixture was concentrated under reduced pressure to provide the crude material. The crude was purified by prep-HPLC (column: Welch Xtimate C18 150*30 mm*5 μm; mobile phase:water(FA)-ACN; B %:15%-55%, 30 min) to give 1-[[4-[2-(2-amino-3-pyridyl)-5-phenyl-imidazo[4,5-b]pyridin-3-yl]phenyl]methyl]-4-methyl-piperidine-4-carboxylic acid I-86 (3.60 mg, 6.16% yield) as a yellow solid. ¹H NMR (400 MHz, DMSO-d₆) δ=8.27 (d, 0.1=8.4 Hz, 1H), 8.05-7.97 (m, 4H), 7.50-7.37 (m, 7H), 7.14 (dd, J=1.6, 7.6 Hz, 1H), 7.02 (s, 2H), 6.38 (dd, J=4.8, 7.6 Hz, 1H), 3.54 (d, J=2.4 Hz, 2H), 2.65-2.56 (m, 2H), 2.24-2.07 (m, 2H), 1.98 (br d, J=12.8 Hz, 2H), 1.46-1.36 (m, 2H), 1.12 (s, 3H). LCMS Rₜ=2.137 min in 4 min chromatography, purity 96.164%, MS ESI calcd. For 518.24 [M+H]⁺ 519.24, found 519.2. HPLC Rₜ=3.447 min in 8 min chromatography, purity 100.00%.

### Example 78. Synthesis of (1R,3R)-3-((2-(4-(2-(2-aminopyridin-3-yl)-5-phenyl-3H-imidazo[4,5-b]pyridin-3-yl)phenyl)propan-2-yl)amino)cyclopentane-1-carboxylic acid (Compound I-87)

### Synthesis of tert-butyl (2-(4-aminophenyl)propan-2-yl)carbamate

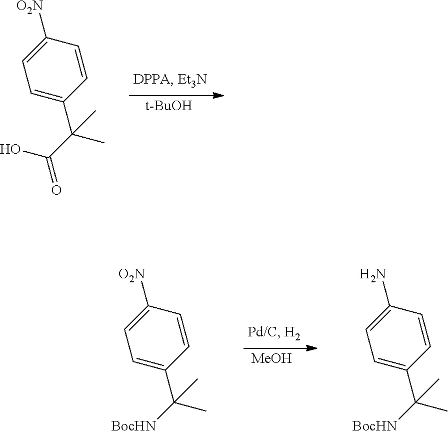 [SMILES: "t-BuOH" ; "MeOH" ; CC(C)(C(=O)O)c1ccc([N+](=O)[O-])cc1 ; CC(C)(C)OC(=O)NC(C)(C)c1ccc([N+](=O)[O-])cc1 ; CC(C)(C)OC(=O)NC(C)(C)c1ccc(N)cc1]

Step 1. To a solution of 2-methyl-2-(4-nitrophenyl)propanoic acid (10.0 g, 47.8 mmol) in tert-butanol (100 mL), TEA (14.5 g, 143 mmol) and DPPA (19.7 g, 71.7 mmol) were added and the mixture was stirred at 90° C. for 12 hrs. Water (100 mL) was added to the reaction mixture and the mixture was extracted with EtOAc (100 mL×3). The combined organic phase was washed with brine (30 mL), dried over anhydrous Na₂SO₄, filtered and concentrated. The residue was purified by flash silica gel chromatography (eluent of 0-6% EtOAc in PE) to give tert-butyl (2-(4-nitrophenyl)propan-2-yl)carbamate (7.20 g, 53.7% yield) as a yellow solid. ¹H NMR (400 MHz, DMSO-d₆) δ=8.16 (d, J=8.4 Hz, 2H), 7.59 (d, J=8.8 Hz, 2H), 7.47-7.35 (m, 1H), 1.51 (s, 6H), 1.33 (s, 9H).

Step 2. To a solution of tert-butyl (2-(4-nitrophenyl)propan-2-yl)carbamate (7.20 g, 25.7 mmol) in methanol (20 mL), Pd/C (10%, 2.0 g) was added under N₂ atmosphere. The suspension was degassed and purged with H₂ (×3), and the reaction mixture was stirred under H₂ (15 psi) at 25° C. for 12 hrs. The reaction mixture was filtered, and the filtrate was concentrated to give tert-butyl (2-(4-aminophenyl)propan-2-yl)carbamate (6.10 g, 94.9% yield) as a yellow oil, which was used in the next step without further purification. ¹H NMR (400 MHz, DMSO-d₆) δ=6.98 (d, J=8.4 Hz, 2H), 6.90-6.79 (m, 1H), 6.47 (d, J=8.4 Hz, 2H), 4.82 (s, 2H), 1.43 (s, 6H), 1.39-1.16 (m, 9H).

### Synthesis of (1R,3R)-3-((2-(4-(2-(2-aminopyridin-3-yl)-5-phenyl-3H-imidazo[4,5-b]pyridin-3-yl)phenyl)propan-2-yl)amino)cyclopentane-1-carboxylic acid (Compound I-87)

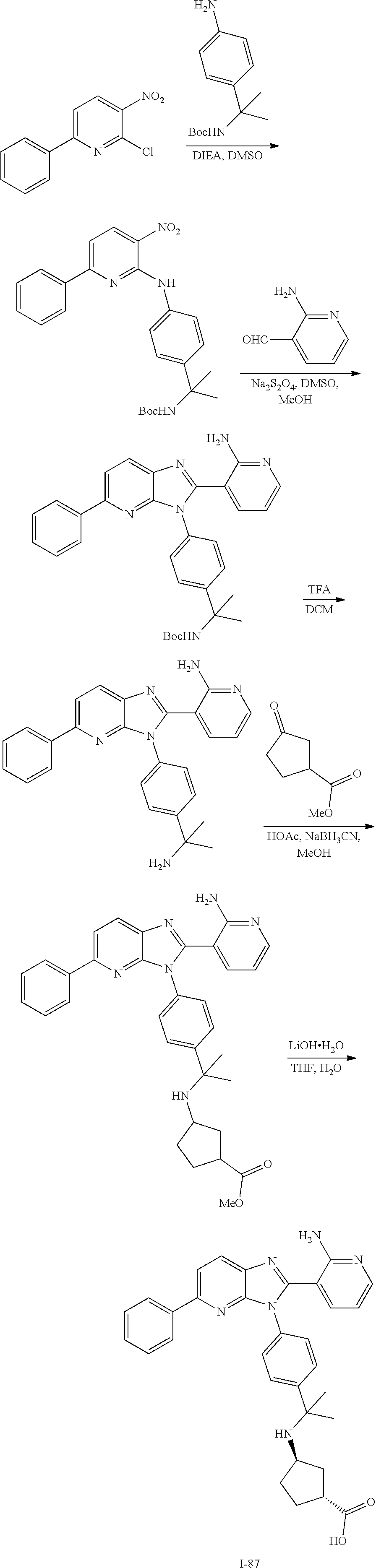

Step 1. To a solution of 2-chloro-3-nitro-6-phenylpyridine (6.00 g, 25.6 mmol) in DMSO (60 mL), DIEA (3.30 g, 25.6 mmol) and tert-butyl (2-(4-aminophenyl)propan-2-yl)carbamate (6.40 g, 25.6 mmol) were added and the mixture was stirred at 80° C. for 12 hrs. Water (50 mL) was added to the reaction mixture and the mixture was extracted with EtOAc (200 mL×2). The combined organic phase was washed with brine (50 mL), dried over anhydrous Na₂SO₄, filtered and concentrated. The residue was purified by flash silica gel chromatography (eluent of 0-10% EtOAc in PE) to give tert-butyl (2-(4-((3-nitro-6-phenylpyridin-2-yl)amino)phenyl)propan-2-yl)carbamate (12.0 g, crude) as a red solid. LCMS Rₜ=0.979 min in 2.0 min chromatography, purity 97.30%, MS ESI calcd. for 448.21 [M+H]⁺ 449.21, found 449.2.

Step 2. To a solution of tert-butyl (2-(4-((3-nitro-6-phenylpyridin-2-yl)amino)phenyl)propan-2-yl)carbamate (12.0 g, 26.8 mmol) and 2-aminopyridine-3-carbaldehyde (6.54 g, 53.5 mmol) in DMSO (100 mL) and methanol (100 mL), Na₂S₂O₄ (32.6 g, 187 mmol) was added under N₂ and the mixture was stirred at 100° C. for 12 hrs. The reaction mixture was concentrated. Water (100 mL) was added to the residue and the resulting mixture was extracted with EtOAc (200 mL×3). The combined organic phase was washed with brine (50 mL), dried over anhydrous Na₂SO₄, filtered and concentrated to give tert-butyl (2-(4-(2-(2-aminopyridin-3-yl)-5-phenyl-3H-imidazo[4,5-b]pyridin-3-yl)phenyl)propan-2-yl)carbamate (4.10 g, 29.4% yield) as a brown solid, which was used in the next step without further purification. ¹H NMR (400 MHz, DMSO-d₆) δ=9.84 (s, 1H), 8.27-8.25 (m, 2H), 8.03 (d, J=7.2 Hz, 5H), 7.51 (d, J=8.4 Hz, 4H), 6.98-6.92 (m, 2H), 6.73 (dd, J=4.8, 7.6 Hz, 1H), 6.34 (br dd, J=5.2, 7.6 Hz, 2H), 1.56 (s, 9H), 1.36 (s, 6H).

Step 3. To a solution of tert-butyl (2-(4-(2-(2-aminopyridin-3-yl)-5-phenyl-3H-imidazo[4,5-b]pyridin-3-yl)phenyl)propan-2-yl)carbamate (1.00 g, 1.92 mmol) in DCM (10 mL), TFA (1 mL, 19.2 mmol) was added and the mixture was stirred at 25° C. for 12 hrs. The reaction mixture was concentrated directly. The mixture was diluted with EtOAc (50 mL) and washed with sat aq. NaHCO₃ (50 mL×3). The combined organic phase was washed with brine (50 mL), dried over anhydrous Na₂SO₄, filtered and concentrated. The residue was purified by flash silica gel chromatography (eluent of 0-5% MeOH in DCM) to give 3-(3-(4-(2-aminopropan-2-yl)phenyl)-5-phenyl-3H-imidazo[4,5-b]pyridin-2-yl)pyridin-2-amine (250 mg, 30.9% yield) as a brown solid. ¹H NMR (400 MHz, DMSO-d₆) δ=8.26 (d, J=8.4 Hz, 1H), 8.05-7.94 (m, 4H), 7.70 (d, J=8.4 Hz, 2H), 7.49-7.37 (m, 5H), 7.17 (dd, J=1.6, 7.6 Hz, 1H), 6.96 (s, 2H), 6.40 (dd, J=4.8, 7.6 Hz, 1H), 1.43 (s, 6H).

Step 4. To a solution of 3-(3-(4-(2-aminopropan-2-yl)phenyl)-5-phenyl-3H-imidazo[4,5-b]pyridin-2-yl)pyridin-2-amine (250 mg, 0.59 mmol) in methanol (5 mL), AcOH (18 mg, 0.29 mmol) and methyl 3-oxocyclopentane-1-carboxylate (101 mg, 0.71 mmol) were added and the mixture was stirred at 25° C. for 30 mins. NaBH₃CN (252 mg, 1.19 mmol) was then added to the mixture and the mixture was stirred at 70° C. for 12 hrs. The reaction mixture was adjusted to pH ~7 by sat aq. NaHCO₃. The resulting mixture was extracted with EtOAc (10 mL×2) and the combined organic phase was washed with brine (20 mL), dried over anhydrous Na₂SO₄, filtered and concentrated. The residue was purified by prep. HPLC (Column: Welch Xtimate C18 40×200 mm 7 μm; mobile phase: [water (NH₄HCO₃)-ACN]; B %: 42%-82%, 25 min) to give methyl 3-((2-(4-(2-(2-aminopyridin-3-yl)-5-phenyl-3H-imidazo[4,5-b]pyridin-3-yl)phenyl)propan-2-yl)amino)cyclopentane-1-carboxylate (90.0 mg, 27.7% yield) as a white solid. ¹H NMR (400 MHz, DMSO-d₆) δ=8.26 (s, 1H), 8.03 (d, J=7.6 Hz, 4H), 7.66 (d, J=2.0 Hz, 2H), 7.49-7.43 (m, 4H), 7.15-7.11 (m, 3H), 7.09 (s, 2H), 6.33-6.31 (m, 1H), 3.58 (s, 3H), 2.93-2.81 (m, 2H), 1.92-1.84 (m, 2H), 1.82-1.71 (m, 2H), 1.68-1.58 (m, 2H), 1.44 (s, 6H).

Step 5. To a solution of methyl 3-((2-(4-(2-(2-aminopyridin-3-yl)-5-phenyl-3H-imidazo[4,5-b]pyridin-3-yl)phenyl)propan-2-yl)amino)cyclopentane-1-carboxylate (80.0 mg, 0.15 mmol) in THF (0.5 mL), LiOH·H₂O (18 mg, 0.439 mmol) and water (0.5 mL) were added and the mixture was stirred at 25° C. for 2 hrs. The reaction mixture was filtered, and the filtrate was concentrated to dryness. The residue was purified by prep. HPLC (Column: Welch Xtimate C18 40×200 mm 7 um; mobile phase: [water (NH₄HCO₃)-ACN]; B %: 8%-48%, 25 min) to give (1R,3R)-3-((2-(4-(2-(2-aminopyridin-3-yl)-5-phenyl-3H-imidazo[4,5-b]pyridin-3-yl)phenyl)propan-2-yl)amino)cyclopentane-1-carboxylic acid I-87 (33.0 mg, 40.7% yield) as a white solid. ¹H NMR (400 MHz, DMSO-d₆) δ=8.27 (d, J=8.4 Hz, 1H), 8.06-7.94 (m, 4H), 7.67 (d, J=8.4 Hz, 2H), 7.49-7.36 (m, 5H), 7.15-7.05 (m, 3H), 6.32 (dd, J=4.8, 7.6 Hz, 1H), 2.94-2.84 (m, 1H), 2.77-2.69 (m, 1H), 1.88-1.75 (m, 2H), 1.64-1.46 (m, 3H), 1.43 (d, J=5.6 Hz, 6H), 1.23-1.12 (m, 1H). HPLC Rₜ=2.204 min in 8 min chromatography, purity 97.66%. LCMS Rₜ=0.979 min in 2.0 min chromatography, purity 97.30%, MS ESI calcd. for 532.26 [M+H]⁺ 533.26, found 533.2.

### Example 79. Synthesis of (1S,3R)-3-((2-(4-(2-(2-aminopyridin-3-yl)-5-phenyl-3H-imidazo[4,5-b]pyridin-3-yl)phenyl)propan-2-yl)amino)cyclopentane-1-carboxylic acid (Compound I-88)

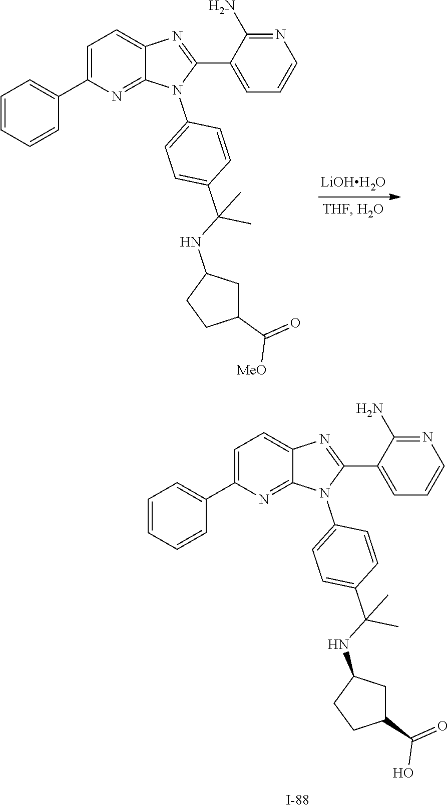

Step 1. To a solution of methyl 3-((2-(4-(2-(2-aminopyridin-3-yl)-5-phenyl-3H-imidazo[4,5-b]pyridin-3-yl)phenyl)propan-2-yl)amino)cyclopentane-1-carboxylate (80.0 mg, 0.15 mmol) in THF (0.5 mL), LiOH·H₂O (18 mg, 0.439 mmol) and water (0.5 mL) were added and the mixture was stirred at 25° C. for 2 hrs. The reaction mixture was filtered, and the filtrate was concentrated to dryness. The residue was purified by prep. HPLC (Column: Welch Xtimate C18 40×200 mm 7 um; mobile phase: [water (NH₄HCO₃)-ACN]; B %: 8%-48%, 25 min) to give (1S,3R)-3-((2-(4-(2-(2-aminopyridin-3-yl)-5-phenyl-3H-imidazo[4,5-b]pyridin-3-yl)phenyl)propan-2-yl)amino)cyclopentane-1-carboxylic acid 1-88 (40.0 mg, 49.9% yield) as a white solid. ¹H NMR (400 MHz, DMSO-d₆) δ=8.27 (d, J=8.4 Hz, 1H), 8.06-7.94 (m, 4H), 7.69 (d, J=8.4 Hz, 2H), 7.49-7.36 (m, 5H), 7.15-7.03 (m, 3H), 6.32 (dd, J=4.8, 7.6 Hz, 1H), 2.90-2.84 (m, 1H), 2.61-2.53 (m, 1H), 1.80-1.70 (m, 3H), 1.56-1.42 (m, 8H), 1.39-1.32 (m, IH). HPLC Rₜ=2.244 min in 8 min chromatography, purity 97.38%. LCMS Rₜ=0.979 min in 2.0 min chromatography, purity 93.52%, MS ESI calcd. for 532.26 [M+H]⁺ 533.26, found 533.2.

### Example 80. Synthesis of (1r,4r)-4-((2-(4 (2-(2-aminopyridin-3-yl)-5-phenyl-3H-imidazo[4,5-b]pyridin-3-yl)phenyl)propan-2-yl)amino)cyclohexane-1-carboxylic acid (Compound I-89)

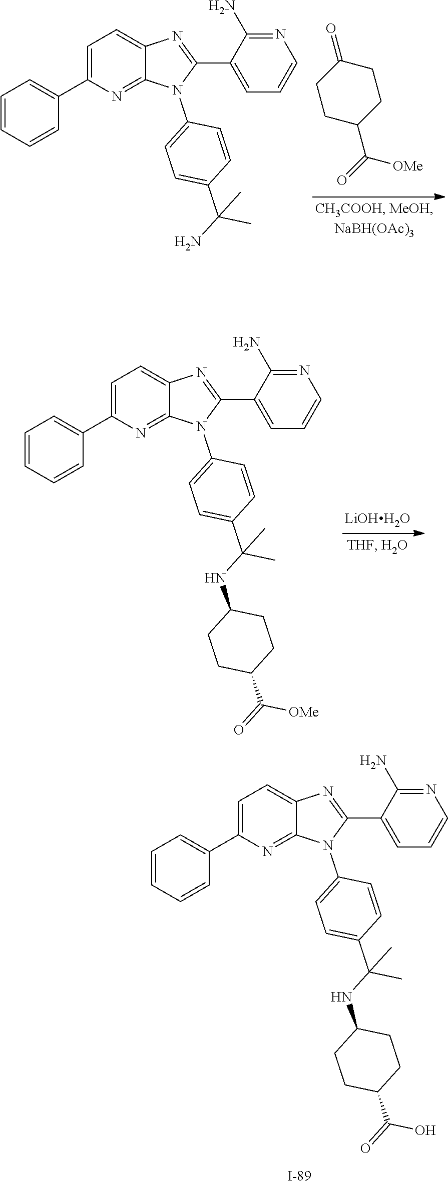

Step 1. To a solution of 3-(3-(4-(2-aminopropan-2-yl)phenyl)-5-phenyl-3H-imidazo[4,5-b]pyridin-2-yl)pyridin-2-amine (300 mg, 0.71 mmol) in methanol (2 mL), methyl 4-oxocyclohexanecarboxylate (167 mg, 1.07 mmol) and AcOH (21 mg, 0.357 mmol) were added and the mixture was stirred at 25° C. for 1 hr. NaBH₃CN (454 mg, 2.14 mmol) was then added and the mixture was stirred at 70° C. for 12 hrs. The reaction mixture was adjusted to pH ~7 by sat aq. NaHCO₃. The resulting mixture was extracted with EtOAc (20 mL×2). The combined organic phase was washed with brine (5 mL), dried over anhydrous Na₂SO₄, filtered and concentrated. The residue was purified by prep-HPLC (Column: Welch Xtimate C18 40*200 mm 7 μm; mobile phase: [water (NH₄HCO)-ACN];B %: 42%-82%,25 min) to give methyl (1r,4r)-4-((2-(4-(2-(2-aminopyridin-3-yl)-5-phenyl-3H-imidazo[4,5-b]pyridin-3-yl)phenyl)propan-2-yl)amino)cyclohexane-1-carboxylate (40.0 mg, 7.55% yield) as a white solid. ¹H NMR (400 MHz, DMSO-d₆) δ=8.27 (d, J=8.4 Hz, 1H), 8.03-7.97 (m, 4H), 7.68 (d, J=8.4 Hz, 2H), 7.47-7.38 (m, 5H), 7.18-7.08 (m, 3H), 6.30 (dd, J=4.8, 7.6 Hz, 1H), 3.56 (s, 3H), 2.17-2.13 (m, 1H), 1.93 (s, 1H), 1.78 (br d, J=12.4 Hz, 2H), 1.56 (dd, J=2.0, 13.2 Hz, 2H), 1.44 (s, 6H), 1.25-1.18 (m, 2H), 1.05-0.97 (m, 2H).

Step 2. To a solution of trans-methyl 4-[[1-[4-[2-(2-amino-3-pyridyl)-5-phenyl-imidazo[4,5-b]pyridin-3-yl]phenyl]-1-methyl-ethyl]amino]cyclohexanecarboxylate (40.0 mg, 0.071 mmol) in THF (0.50 mL), LiOH·H₂O (9.0 mg, 0.214 mmol) and water (0.50 mL) were added and the mixture was stirred at 25° C. for 2 hrs. The reaction mixture was filtered, and the filtrate was concentrated to dryness. The residue was purified by prep-HPLC (Column: Welch Xtimate C18 40×200 mm 7 μm; mobile phase: [water (NH₄HCO₃)-ACN]; B %: 6/6-46%, 25 min) to give (1r,4r)-4-((2-(4-(2-(2-aminopyridin-3-yl)-5-phenyl-3H-imidazo[4,5-b]pyridin-3-yl)phenyl)propan-2-yl)amino)cyclohexane-1-carboxylic acid I-89 (33.0 mg, 83.8% yield) as a white solid. The trans configuration was randomly assigned. ¹H NMR (400 MHz, DMSO-d₆) δ=8.27 (d, J=8.4 Hz, 1H), 8.05-7.96 (m, 4H), 7.69 (d, J=8.4 Hz, 2H), 7.50-7.35 (m, 5H), 7.17-7.08 (m, 3H), 6.31 (dd, J=4.8, 7.6 Hz, 1H), 2.22-2.13 (m, 1H), 2.08-1.99 (m, 1H), 1.78 (d, J=12.0 Hz, 2H), 1.60-1.54 (m, 2H), 1.45 (s, 6H), 1.25-1.15 (m, 2H), 1.06-0.93 (m, 2H). HPLC Rₜ=2.187 min in 8 min chromatography, purity 99.92%. LCMS Rₜ=0.969 min in 2.0 min chromatography, purity 100.00%, MS ESI calcd. for 546.27 [M+H]⁺ 547.27, found 547.2.

### Example 81. Synthesis of (1r,4r)-4-((4-(2-(2-aminopyridin-3-yl)-5-phenyl-3H-imidazo[4,5-b]pyridin-3-yl)benzyl)amino)cyclohexane-1-carboxylic acid (Compound I-90)

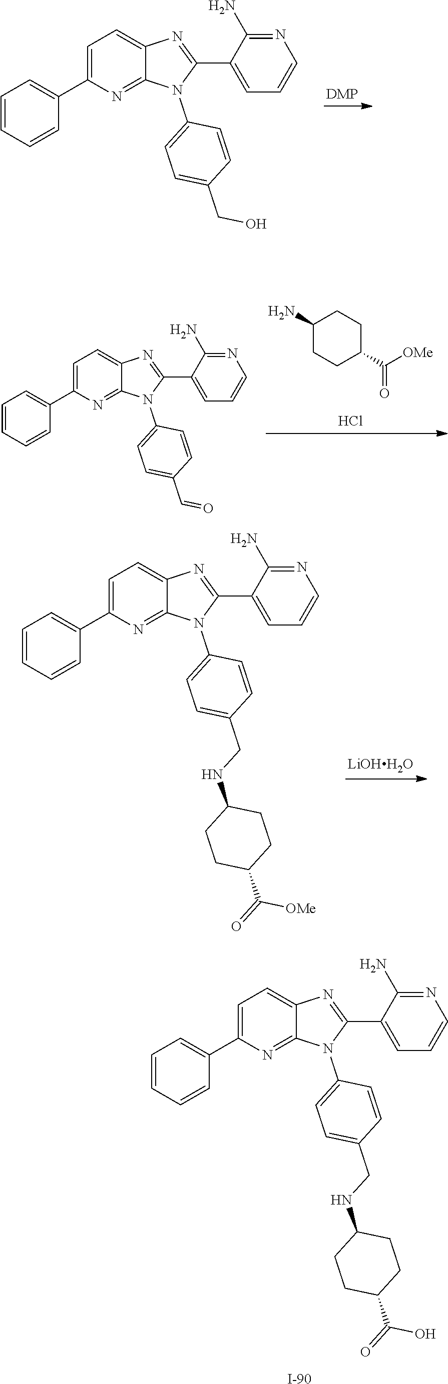

Step 1. To a solution of [4-[2-(2-amino-3-pyridyl)-5-phenyl-imidazo[4,5-b]pyridin-3-yl]phenyl]methanol (80.0 mg, 0.203 mmol) in DCM (3.00 mL), DMP (172 mg, 0.407 mmol) was added at 0° C. and the mixture was stirred at 25° C. for 2 hrs. The mixture was quenched with 1:1 (1M Na₂S₂O₃:NaHCO₃), and further stirred for 30 mins. Water (50 mL) was added and the mixture was extracted with DCM (50 mL×3). The organic phase was washed with brine (50 mL), dried over Na₂SO₄, filtered and concentrated under reduced pressure to give 4-[2-(2-amino-3-pyridyl)-5-phenyl-imidazo[4,5-b]pyridin-3-yl]benzaldehyde (146 mg, crude) as a yellow solid. The crude was directly used in the next step without purification. ¹H NMR (400 MHz, DMSO-d₆) δ=10.09 (s, 1H), 8.30 (d, J=8.4 Hz, 1H), 8.11-8.01 (m, 7H), 7.75 (d, J=8.4 Hz, 2H), 7.49-7.38 (m, 4H), 7.27 (dd, J=1.6, 7.6 Hz, 1H), 6.85 (s, 2H), 6.46 (dd, J=4.8, 7.6 Hz, 1H).

Step 2. To a solution of 4-[2-(2-amino-3-pyridyl)-5-phenyl-imidazo[4,5-b]pyridin-3-yl]benzaldehyde (80.0 mg, 0.204 mmol) in DCM (5.00 mL), trans-methyl 4-aminocyclohexanecarboxylate;hydrochloride (198 mg, 1.02 mmol) in methanol (2.00 mL) and TEA (2.0 mL, 0.204 mmol) were added at 25° C. under N₂. The mixture was stirred at 25° C. for 2 hrs. NaBH₃CN (39.0 mg, 0.613 mmol) was then added and the mixture was stirred at 40° C. for 16 hrs. Water (100 mL) was added and the mixture was extracted with EtOAc (100 mL×3). The organic phase was washed with brine (100 mL), dried over Na₂SO₄, filtered, and concentrated under reduced pressure to provide the crude material. 1M NaOH (20 mL) and NaClO (40 mL) were added to the water phase (pH ~13). The crude was purified by flash chromatography eluting with EtOAc/MeOH (from 0% to 5% of MeOH in EtOAc) to give methyl (1r,4r)-4-((4-(2-(2-aminopyridin-3-yl)-5-phenyl-31-imidazo[4,5-b]pyridin-3-yl)benzyl)amino)cyclohexane-1-carboxylate (58.0 mg, 53.3% yield) as a white solid. ¹H NMR (400 MHz, DMSO-d₆) δ=8.27 (d, J=8.4 Hz, 1H), 8.04-7.97 (m, 4H), 7.53 (d, J=8.0 Hz, 2H), 7.48-7.38 (m, 5H), 7.20 (dd, J=1.6, 7.6 Hz, 1H), 7.05-6.97 (m, 2H), 6.39 (dd, J=4.8, 7.8 Hz, 1H), 3.86 (s, 2H), 3.59 (s, 3H), 2.30-2.22 (m, 1H), 2.04-1.86 (m, 5H), 1.50-1.43 (m, 1H), 1.21-1.05 (m, 3H).

Step 3. To a solution of methyl (1r,4r)-4-((4-(2-(2-aminopyridin-3-yl)-5-phenyl-3H-imidazo[4,5-b]pyridin-3-yl)benzyl)amino)cyclohexane-1-carboxylate (58.0 mg, 0.109 mmol) in THF (1.00 mL) and water (1.00 mL), LiOH·H₂O (11.0 mg, 0.272 mmol) was added at 25° C. and the mixture was stirred for 3 hrs. The mixture was extracted with EtOAc (10 mL). HCl (0.5 mL) was added to the aqueous phase and the aqueous phase was purified by prep-HPLC (Xtimate C18 150*40 mm*5 μm water(FA)-ACN) to give (1r,4r)-4-((4-(2-(2-aminopyridin-3-yl)-5-phenyl-3H-imidazo[4,5-b]pyridin-3-yl)benzyl)amino)cyclohexane-1-carboxylic acid I-90 (15.0 mg, 26.0% yield) as a yellow solid. ¹H NMR (400 MHz, DMSO-d₆) δ=8.26 (d, J=8.4 Hz, 1H), 8.04-7.97 (m, 4H), 7.54-7.36 (m, 7H), 7.19 (dd, J=1.8, 7.4 Hz, 1H), 7.01 (s, 2H), 6.39 (dd, J=4.8, 7.8 Hz, 1H), 3.85 (s, 2H), 2.44-2.38 (m, 1H), 2.17-2.10 (m, 1H), 1.99-1.87 (m, 4H), 1.36-1.25 (m, 2H), 1.15-1.05 (m, 2H). HPLC Rₜ=2.182 min in 8.0 min chromatography, 10-80AB_8 min.1 cm, purity 100.000%. LCMS Rₜ=1.094 min in 4.0 min chromatography, purity 100.00%, MS ESI calcd for 518.24 [M+H]+ 519.24, found 519.1.

### Example 82. Synthesis of (1s,4s)-4-((4-(2-(2-aminopyridin-3-yl)-5-phenyl-3H-imidazo[4,5-b]pyridin-3-yl)benzyl)amino)cyclohexane-1-carboxylic acid (Compound I-91)

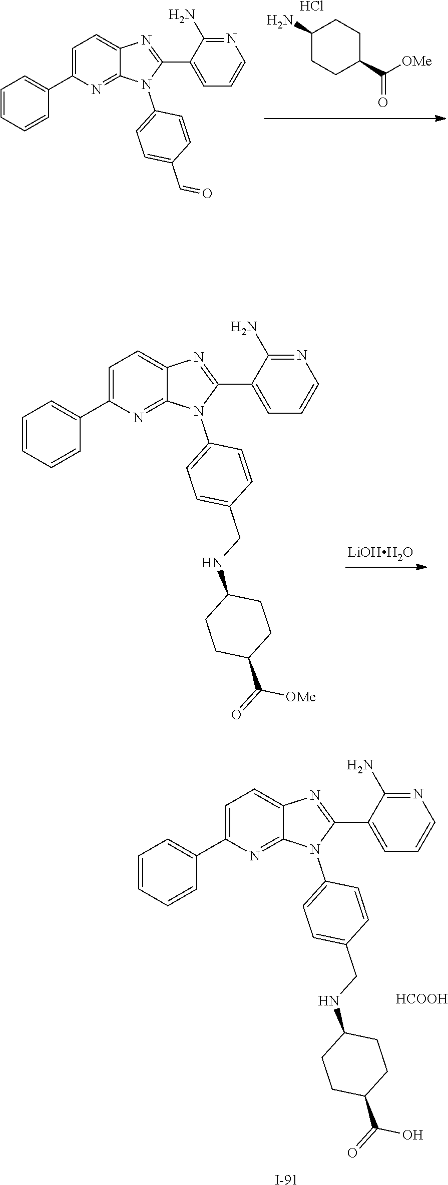

Step 1. To a solution of 4-[2-(2-amino-3-pyridyl)-5-phenyl-imidazo[4,5-b]pyridin-3-yl]benzaldehyde (150 mg, 0.383 mmol) in DCM (5.00 mL), cis-methyl 4-aminocyclohexanecarboxylate;hydrochloride (297 mg, 1.53 mmol) in methanol (2.00 mL) and TEA (2.00 mL, 0.383 mmol) were added at 25° C. under N₂. After 2 hrs, NaBH₃CN (72.0 mg, 1.15 mmol) was added and the mixture was stirred at 40° C. for 16 hrs. Water (100 mL) was added and the mixture was extracted with DCM (100 mL×3). The organic phase was washed with brine (100 mL), dried over Na₂SO₄, filtered, and concentrated under reduced pressure to provide the crude material. 1M NaOH (100 mL) and NaClO (250 mL) were added to the aqueous phase (pH ~13). The crude was purified by flash chromatography eluting with EtOAc/MeOH (from 0% to 5% of MeOH) to give methyl (1s,4s)-4-((4-(2-(2-aminopyridin-3-yl)-5-phenyl-3H-imidazo[4,5-b]pyridin-3-yl)benzyl)amino)cyclohexane-1-carboxylate (125 mg, 61.2% yield) as a white solid. ¹H NMR (400 MHz, DMSO-d₆) δ=8.26 (d, J=8.4 Hz, 1H), 8.05-7.96 (m, 4H), 7.53-7.38 (m, 7H), 7.18 (dd, J=1.6, 7.6 Hz, 1H), 7.02 (s, 2H), 6.38 (dd, J=4.8, 7.6 Hz, 1H), 3.78 (s, 2H), 3.60 (s, 3H), 2.61 (br dd, J=2.8, 6.4 Hz, 1H), 2.45 (br dd, J=3.6, 7.6 Hz, 1H), 1.96-1.87 (m, 2H), 1.67-1.39 (m, 6H).

Step 2. To a solution of methyl (1s,4s)-4-((4-(2-(2-aminopyridin-3-yl)-5-phenyl-3H-imidazo[4,5-b]pyridin-3-yl)benzyl)amino)cyclohexane-1-carboxylate (125 mg, 0.235 mmol) in THF (1.00 mL) and water (1.00 mL), LiOH·H₂O (25.0 mg, 0.587 mmol) was added at 25° C. and the mixture was stirred for 3 hrs. The mixture was extracted with EtOAc (10 mL). HC (0.5 mL) was added to the aqueous phase. The aqueous phase was purified by prep-HPLC (Xtimate C18 150*40 mm*5 um, water (FA)-ACN) to give (1s,4s)-4-((4-(2-(2-aminopyridin-3-yl)-5-phenyl-3H-imidazo[4,5-b]pyridin-3-yl)benzyl)amino)cyclohexane-1-carboxylic acid 1-91 (40.0 mg, 32.5% yield) as a yellow solid. ¹H NMR (400 MHz, DMSO-d₆) δ=8.26 (d, J=8.4 Hz, 1H), 8.22 (s, 1H), 8.04-7.97 (m, 4H), 7.54 (d, J=8.4 Hz, 2H), 7.49-7.42 (m, 4H), 7.42-7.37 (m, IH), 7.19 (dd, J=1.6, 7.6 Hz, 1H), 7.01 (s, 2H), 6.39 (dd, J=5.0, 7.6 Hz, 1H), 3.86 (s, 2H), 2.45-2.30 (m, 2H), 1.97-1.89 (m, 2H), 1.63 (br t, J=8.8 Hz, 2H), 1.52-1.43 (m, 4H). HPLC Rₜ=2.274 min in 8.0 min chromatography, 10-80AB_8 min.1 cm, purity 100%. LCMS Rₜ=1.907 min in 4.0 min chromatography, purity 100.00%, MS ESI calcd. for 518.24 [M+H]+ 519.24, found 519.1.

### Example 83. Synthesis of 2-(1-(4-(2-(2-aminopyridin-3-yl)-5-phenyl-3H-imidazo[4,5-b]pyridin-3-yl)benzyl)piperidin-4-yl)-2-methylpropanoic acid (Compound I-92)

### Synthesis of methyl 2-methyl-2-(piperidin-4-yl)propanoate

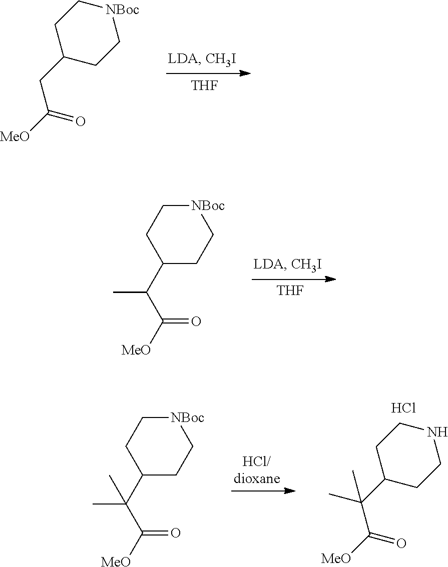

Step 1. To a stirred solution of tert-butyl 4-(2-methoxy-2-oxo-ethyl)piperidine-1-carboxylate (1.00 g, 3.89 mmol) in THF (10 mL), LDA (2M, 7.5 mL) was added slowly at −78° C. and the mixture was stirred at −78° C. under N₂ for 1 hr. MeI (1.2 mL, 19.4 mmol) was then added to the reaction mixture and the resulting mixture was stirred at 20° C. under N₂ for 1 hr. The reaction mixture was quenched with water (20 mL) and extracted with EtOAc (20 mL×2). The combined organic layers were washed with brine (20 mL), dried over anhydrous sodium sulfate, filtered and the filtrate was concentrated to afford a residue. The crude product was purified by flash chromatography on silica gel (0-30% EtOAc) to afford tert-butyl 4-(2-methoxy-1-methyl-2-oxo-ethyl)piperidine-1-carboxylate (900 mg, 85.4% yield) as a yellow oil. ¹H NMR (400 MHz, DMSO-d₆) δ=3.98 (s, 2H), 3.63 (s, 3H), 2.80-2.61 (m, 2H), 2.33 (t, J=7.2 Hz, IH), 1.70-1.59 (m, 2H), 1.56-1.49 (m, 1H), 1.42 (s, 9H), 1.13-0.96 (m, 6H).

Step 2. To a stirred solution of tert-butyl 4-(2-methoxy-1-methyl-2-oxo-ethyl)piperidine-1-carboxylate (900 mg, 3.32 mmol) in THF (10 mL), LDA (5.0 mL, 9.95 mmol) was added slowly at −78° C. and the mixture was stirred at −78° C. under N₂ for 1 hr. MeI (1.0 mL, 16.6 mmol) was then added to the reaction mixture and the resulting mixture was stirred at 20° C. under N₂ for 1 hr. The reaction mixture was quenched with water (20 mL) and extracted with EtOAc (20 mL, 2). The combined organic layers were washed with brine (20 mL), dried over anhydrous sodium sulfate, filtered and the filtrate was concentrated to afford a residue. The crude product was purified by flash chromatography on silica gel (0-30% of EtOAc in PE) to afford tert-butyl 4-(1-methoxy-2-methyl-1-oxopropan-2-yl)piperidine-1-carboxylate (600 mg, 63.4% yield) as yellow oil ¹H NMR (400 MHz, DMSO-d₆) δ=4.07-3.96 (m, 2H), 3.63 (s, 3H), 3.51-3.45 (m, 2H), 1.74-1.63 (m, 1H), 1.50 (d, J=12.8 Hz, 2H), 1.42 (s, 9H), 1.14-1.05 (m, 8H).

Step 3. To a solution of tert-butyl 4-(2-methoxy-1,1-dimethyl-2-oxo-ethyl)piperidine-1-carboxylate (600 mg, 2.10 mmol) in 1,4-dioxane (2 mL), HCl/dioxane (6.0 mL) was added under N₂ atmosphere and the mixture was stirred at 20° C. for 6 hrs. The reaction mixture was concentrated to give methyl 2-methyl-2-(4-piperidyl)propanoate (350 ng, 89.9% yield) as a white solid. ¹H NMR (400 MHz, DMSO-d₆) δ=9.11-8.51 (m, 1H), 3.61 (s, 3H), 3.26 (d, J=12.0 Hz, 2H), 2.80 (d, J=10.8 Hz, 2H), 1.78 (d, J=12.0 Hz, 1H), 1.61 (d, J=13.2 Hz, 2H), 1.45 (d, J=12.4 Hz, 2H), 1.07 (s, 6H).

### Synthesis of 2-(1-(4-(2-(2-aminopyridin-3-yl)-5-phenyl-3H-imidazo[4,5-b]pyridin-3-yl)benzyl)piperidin-4-yl)-2-methylpropanoic acid (Compound I-92)

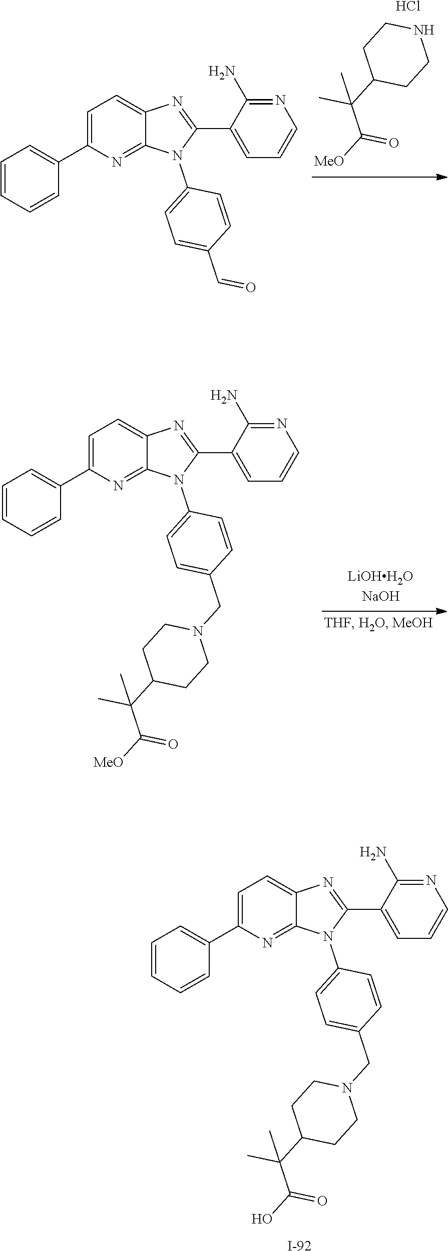

Step 1. To a solution of methyl 2-methyl-2-(4-piperidyl)propanoate (142 mg, 0.766 mmol) in methanol (3 mL), TEA (3.0 mL, 0.255 mmol) (pH ~7) was added. 4-[2-(2-Amino-3-pyridyl)-5-phenyl-imidazo[4,5-b]pyridin-3-yl]benzaldehyde (100 mg, 0.255 mmol) was then added to the mixture and the mixture was stirred at 25° C. for 1 hr. NaBH₃CN (48 mg, 0.766 mmol) was then added and the mixture was stirred at 25° C. for 16 hrs. Water (20 mL) was then added and the mixture was extracted with EtOAc (20 mL×3). The organic phase was washed with brine (20 mL), dried over Na₂SO₄, filtered, and concentrated under reduced pressure to provide the crude material. The crude was purified by flash chromatography eluting with DCM/MeOH (from 0% to 30% of MeOH) to give methyl 2-[1-[[4-[2-(2-amino-3-pyridyl)-5-phenyl-imidazo[4,5-b]pyridin-3-yl]phenyl]methyl]-4-piperidyl]-2-methyl-propanoate (70.0 mg, 48.9% yield) as a yellow oil. ¹H NMR (400 MHz, DMSO-d₆) δ=8.27 (d, J=8.4 Hz, 1H), 8.05-7.96 (m, 4H), 7.49-7.37 (m, 7H), 7.16 (dd, J=1.6, 7.6 Hz, 1H), 7.01 (s, 2H), 6.38 (dd, J=4.8, 7.6 Hz, 1H), 4.09 (q, J=5.2 Hz, 1H), 4.04-4.01 (m, 1H), 3.59 (s, 3H), 3.05 (d, J=6.0 Hz, 2H), 2.93 (d, J=11.6 Hz, 2H), 1.23 (s, 2H), 1.17 (dt, J=4.8, 7.2 Hz, 3H), 1.07 (d, J=3.2 Hz, 6H).

Step 2. To a solution of methyl 2-[1-[[4-[2-(2-amino-3-pyridyl)-5-phenyl-imidazo[4,5-b]pyridin-3-yl]phenyl]methyl]-4-piperidyl]-2-methyl-propanoate (70.0 mg, 0.125 mmol) in THF (1 mL), water (0.3 mL) and methanol (1 mL), LiOH·H₂O (15 mg, 0.624 mmol) and NaOH (30 mg, 0.749 mmol) were added under N₂ atmosphere and the mixture was stirred at 60° C. for 12 hrs. The reaction mixture was concentrated. Water (1 mL) was added to the reaction mixture. The reaction mixture was then adjusted to pH ~3 by aq. FA (1 N). The crude product was purified by prep-HPLC(column Welch Xtimate C18 200*40 mm*7 μm; mobile phase: [water(NH₄HCO₃)-ACN];B %: 4%-44%,25 min) to give 2-[1-[[4-[2-(2-amino-3-pyridyl)-5-phenyl-imidazo[4,5-b]pyridin-3-yl]phenyl]methyl]-4-piperidyl]-2-methyl-propanoic acid 1-92 (6.30 mg, 8.89% yield) was obtained as a white solid. ¹H NMR (400 MHz, DMSO-d₆) δ=8.27 (d, J=8.4 Hz, 1H), 8.05-7.96 (m, 4H), 7.50-7.37 (m, 7H), 7.15 (dd, J=1.6, 7.6 Hz, 1H), 7.02 (s, 2H), 6.38 (dd, J=4.8, 7.6 Hz, 1H), 3.53 (s, 2H), 2.95-2.87 (m, 2H), 1.98-1.83 (m, 2H), 1.54-1.45 (m, 3H), 1.29 (dd, J=3.2, 9.2 Hz, 2H), 1.02 (s, 6H). HPLC Rₜ=2.410 min in 8 min chromatography, ACE Excel 3 C18 4.6*100 mm, purity 96.538%. LCMS Rₜ=1.514 min in 4 min chromatography, Agilent PoroShell 120 EC-C18 2.7 um 3.0*50 mm, purity 95.907%, MS ESI calcd. for 546.27 [M+H]⁺ 547.27, found 547.2.

### Example 84. Synthesis of 2-(4-(2-(2-aminopyridin-3-yl)-5-phenyl-3H-imidazo[4,5-b]pyridin-3-yl)benzyl)-2-azaspiro[3.3]heptane-6-carboxylic acid (Compound I-93)

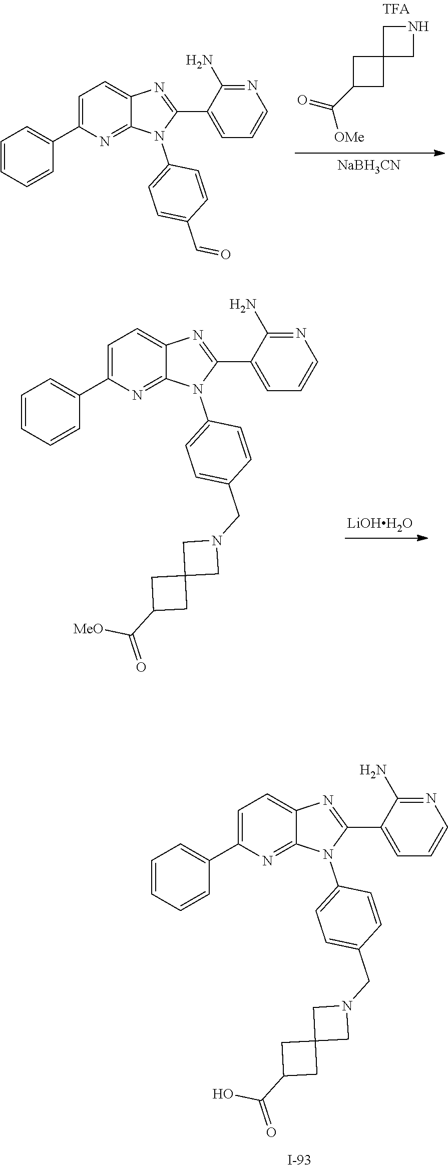

Step 1. To a solution of methyl 2-azaspiro[3.3]heptane-6-carboxylate (69.0 mg, 0.270 mmol) in methanol (3 mL), TEA (21 mg, 0.18 mmol) (pH ~7) was added. 4-(2-(2-Aminopyridin-3-yl)-5-phenyl-3H-imidazo[4,5-b]pyridin-3-yl)benzaldehyde (50 mg, 0.098 mmol) was added and the mixture was stirred at 25° C. for 0.5 hr. NaBH₃CN (18 mg, 0.27 mmol) and AcOH (2 drops) were then added and the mixture was stirred at 25° C. for 16 hrs. The reaction mixture was concentrated directly to afford methyl 2-(4-(2-(2-aminopyridin-3-yl)-5-phenyl-3H-imidazo[4,5-b]pyridin-3-yl)benzyl)-2-azaspiro[3.3]heptane-6-carboxylate (50.0 mg, crude) as a white solid.

Step 2. To a solution of methyl 2-(4-(2-(2-aminopyridin-3-yl)-5-phenyl-3H-imidazo[4,5-b]pyridin-3-yl)benzyl)-2-azaspiro[3.3]heptane-6-carboxylate (50 mg, 0.09 mmol) in THF (2 mL) and water (2 mL), LiOH·H₂O (40 mg, 0.94 mmol) was added at 25° C. and the mixture was stirred for 14 hrs. FA (2 mL) was added to the mixture. The residue was purified by prep-HPLC (condition: water (FA)-ACN; Column: Xtimate C18 40*200 mm*7 μm; B %: 0%-34%; Gradient Time, 25 min) to afford 2-(4-(2-(2-aminopyridin-3-yl)-5-phenyl-3H-imidazo[4,5-b]pyridin-3-yl)benzyl)-2-azaspiro[3.3]heptane-6-carboxylic acid I-93 (4.10 mg, 8.42% yield) as a white solid. ¹H NMR (400 MHz, DMSO-d₆) δ=8.27 (d, J=8.4 Hz, 1H), 8.04-7.97 (m, 4H), 7.51-7.37 (m, 7H), 7.16 (dd, J=2.0, 7.6 Hz, 1H), 6.98 (s, 2H), 6.40 (dd, J=4.4, 7.6 Hz, 1H), 3.26-3.16 (m, 6H), 2.91 (t, J=8.0 Hz, 1H), 2.37-2.34 (m, 1H), 2.31-2.22 (m, 3H). HPLC Rₜ=1.789 min in 8 min chromatography, purity 100.00%. LCMS Rₜ=1.323 min in 4 min chromatography, purity 100.00%, MS ESI calcd. for 516.23 [M+H]⁺ 517.23, found 517.2.

### Example 85. Synthesis of 2-(1-(4-(2-(2-aminopyridin-3-yl)-5-phenyl-3H-imidazo[4,5-b]pyridin-3-yl)benzyl)pyrrolidin-3-yl)acetic acid (Compound I-94)

### Synthesis of methyl 2-(pyrrolidin-3-yl)acetate

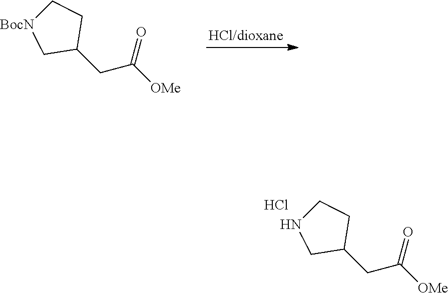 [SMILES: COC(=O)CC1CCN(C(=O)OC(C)(C)C)C1 ; Cl ; COC(=O)CC1CCNC1]

Step 1. To a solution of tert-butyl 3-(2-methoxy-2-oxo-ethyl)pyrrolidine-1-carboxylate (200 mg, 0.822 mmol) in 1,4-dioxane (3.00 mL), HC/dioxane (3.0 mL, 0.822 mmol) was added at 25° C. and the mixture was stirred for 16 hrs. The mixture was concentrated under reduced pressure to give methyl 2-pyrrolidin-3-ylacetate hydrochloride (188 mg, crude) as a colorless oil, which was used directly according to the theoretical amount. ¹H NMR (400 MHz, DMSO-d₆) δ=3.58-3.52 (m, 3H), 3.43-3.37 (m, 1H), 3.29 (m, 1H), 3.23-3.15 (m, 1H), 3.12-3.03 (m, 1H), 2.75 (m, 1H), 2.54-2.51 (m, 2H), 2.11-2.02 (m, 1H), 1.58-1.49 (m, 1H).

### Synthesis of 2-(1-(4-(2-(2-aminopyridin-3-yl)-5-phenyl-3H-imidazo[4,5-b]pyridin-3-yl)benzylpyrrolidin-3-yl)acetic acid (Compound I-94)

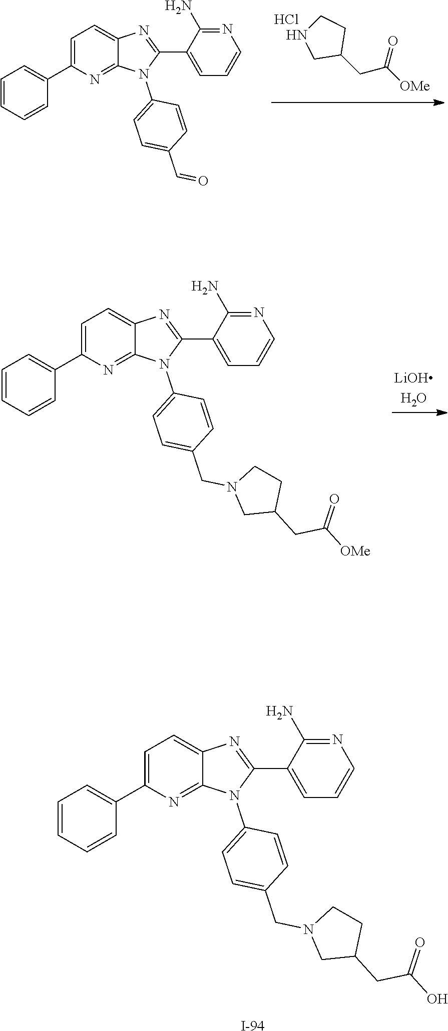

Step 2. To a solution of methyl 2-[1-[[4-[2-(2-amino-3-pyridyl)-5-phenyl-imidazo[4,5-b]pyridin-3-yl]phenyl]methyl]pyrrolidin-3-yl]acetate (52.0 mg, 0.100 mmol) in THF (1.00 mL) and water (1.00 mL), LIOH H₂O (11.0 mg, 0.251 mmol) was added at 25° C. and the mixture was stirred for 16 hrs. The mixture was extracted with EtOAc (10 ml). HCl (0.5 ml) was added to the aqueous phase. The aqueous phase was purified by prep-HPLC (Xtimate C18 150*40 mm*5 μm water (FA)-ACN) to give 2-[I-[[4-[2-(2-amino-3-pyridyl)-5-phenyl-imidazo[4,5-b]pyridin-3-yl]phenyl]methyl]pyrrolidin-3-yl]acetic acid 1-94 (17.0 mg, 32.5% yield) as a yellow solid. ¹H NMR (400 MHz, DMSO-d₆)=8.27 (d, J=8.4 Hz, 1H), 8.06-7.97 (m, 4H), 7.51-7.36 (m, 7H), 7.14 (dd, J=1.6, 7.6 Hz, 1H), 7.03 (s, 2H), 6.39 (dd, J=4.8, 7.6 Hz, 1H), 3.69 (s, 2H), 2.78-2.72 (m, 1H), 2.59-2.54 (m, 2H), 2.45-2.40 (m, 1H), 2.34-2.30 (m, 2H), 2.23-2.16 (m, 1H), 2.05-1.94 (m, 1H), 1.40 (dd, J=6.8, 13.2 Hz, 1H). HPLC Rₜ=2.100 min in 8.0 min chromatography, 10-80AB_8 min.1 cm, purity 98.510%. LCMS Rₜ=1.083 min in 4.0 min chromatography, purity 98.603%, MS ESI calcd. for 504.23, [M+H]⁺ 505.23, found 505.0.

### Example 86. Synthesis of (1s,4s)-4-((4-(2-(2-aminopyridin-3-yl)-5-phenyl-3H-imidazo[4,5-b]pyridin-3-yl)benzyl)(methyl)amino)cyclohexane-1-carboxylic acid (Compound I-95)

### Synthesis of methyl (1s,4s)-4-(methylamino)cyclohexane-1-carboxylate

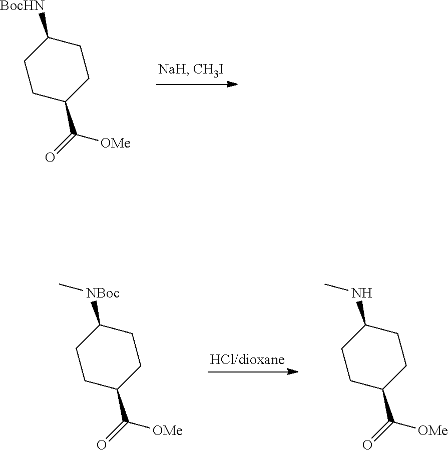

Step 1. To a solution of methyl (1s,4s)-4-((tert-butoxycarbonyl)amino)cyclohexane-1-carboxylate (1.00 g, 3.89 mmol) in DMF (10 mL), NaH (171 mg, 4.27 mmol, 60%) was added at 0° C. under N₂ atmosphere and the mixture was stirred at 0° C. for 0.5 hr. Mel (1.66 g, 11.7 mmol) was then added and the mixture was stirred at 50° C. for 10 hrs. The reaction was quenched with saturated aqueous NH₄Cl (50 mL) dropwise. The resulting mixture was extracted with EtOAc (50 mL×3). The combined organic phase was washed with brine (80 mL), dried over anhydrous Na₂SO₄, filtered and concentrated. The crude product was purified by column chromatography on silica gel (EtOAc in PE, 8%) to give methyl (1s,4s)-4-((tert-butoxycarbonyl)(methyl)amino)cyclohexane-1-carboxylate (400 mg, 37.9% yield) as a colorless oil. ¹H NMR (DMSO-d₆, 400 MHz) β_{H}=3.63 (s, 3H), 2.67-2.62 (m, 1H), 2.60 (s, 3H), 2.12-2.05 (m, 2H), 1.54-1.42 (m, 6H), 1.38 (s, 10H).

Step 2. To a solution of methyl (1 s,4s)-4-((tert-butoxycarbonyl)(methyl)amino)cyclohexane-1-carboxylate (400 mg, 1.47 mmol) in DCM (2 mL), HCl/dioxane (4 mL, 2M) was added at 25° C. and the mixture was stirred for 10 hrs. The reaction mixture was concentrated directly to give methyl (1s,4s)-4-(methylamino)cyclohexane-1-carboxylate (350 mg, crude) as a colorless oil, which was used without further purification. ¹H NMR (DMSO-d₆, 400 MHz) δ_{H}=9.41 (s, 1H), 3.74-3.69 (m, 3H), 3.10-2.97 (m, 1H), 2.69-2.60 (m, 3H), 2.33-2.23 (m, 2H), 2.11-1.98 (m, 3H), 1.90-1.76 (m, 2H), 1.67-1.55 (m, 2H).

### Synthesis of (1s,4s)-4-((4-(2-(2-aminopyridin-3-yl)-5-phenyl-3H-imidazo[4,5-b]pyridin-3-yl)benzyl)(methyl)amino)cyclohexane-1-carboxylic acid (Compound I-95)

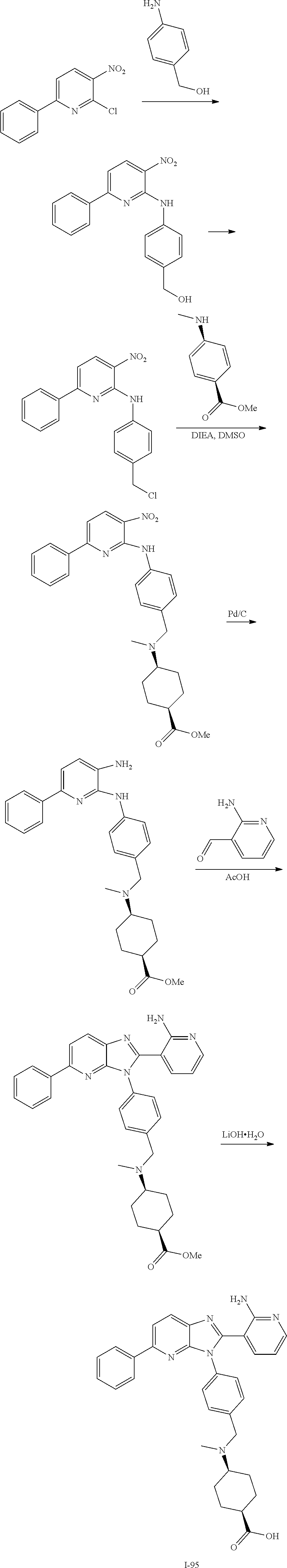

Step 1. To a solution of 2-chloro-3-nitro-6-phenyl-pyridine (10.0 g, 42.6 mmol) in DMSO (100 mL), (4-aminophenyl)menthanol (9.45 g, 76.7 mmol) was added at 25° C. under N₂ and the mixture was stirred at 90° C. for 16 hrs. Water (600 mL) was added and the mixture was filtered. The wet cake was concentrated under reduced pressure to give [4-[(3-nitro-6-phenyl-2-pyridyl)amino]phenyl]methanol (14.6 g, crude) as a red solid. ¹H NMR (400 MHz, DMSO-d₆) δ=10.10 (s, 1H), 8.61 (d, J=8.8 Hz, 1H), 8.10 (d, J=2.0, 4.0 Hz, 2H), 7.70 (d, J=8.4 Hz, 2H), 7.59 (d, J=8.8 Hz, 1H), 7.56-7.52 (m, 3H), 7.37 (d, J=8.4 Hz, 2H), 4.52 (d, J=6.0 Hz, 2H).

Step 2. A mixture of (4-((3-nitro-6-phenylpyridin-2-yl)amino)phenyl)methanol (2.00 g, 6.22 mmol) and SOCl₂ (23 mL, 6.22 mmol) was stirred at 25° C. for 3 hrs. The reaction mixture was concentrated to afford N-(4-(chloromethyl)phenyl)-3-nitro-6-phenylpyridin-2-amine (1.30 g, 61.5% yield) as a yellow solid, which was used in the next step without purification. ¹H NMR (400 MHz, DMSO-d₆) δ=10.18 (s, 1H), 8.67 (d, J=8.8 Hz, 1H), 8.17-8.13 (m, 2H), 7.83 (d, J=8.8 Hz, 2H), 7.66 (d, J=8.4 Hz, 1H), 7.61-7.57 (m, 3H), 7.54 (d, J=8.4 Hz, 2H), 4.85 (s, 2H).

Step 3. To a solution of methyl (1s,4s)-4-(methylamino)cyclohexane-1-carboxylate (252 mg, 1.47 mmol) in DMSO (4 mL), DIEA (285 mg, 2.21 mmol) and N-(4-(chloromethyl)phenyl)-3-nitro-6-phenylpyridin-2-amine (250 mg, 0.736 mmol) were added at 25° C. and the mixture was stirred at 80° C. for 14 hrs. EtOAc (30 mL) was added the reaction and the resulting suspension was washed with brine (20 mL×3), dried over anhydrous Na₂SO₄, filtered and concentrated. The crude product was purified by column chromatography on silica gel (EtOAc in PE, 12%) to give methyl (1s,4s)-4-(methyl(4-((3-nitro-6-phenylpyridin-2-yl)amino)benzyl)amino)cyclohexane-1-carboxylate (354 mg, crude) as a red oil. LCMS Rₜ=1.354 min in 2 min chromatography, Agilent PoroShell 120 EC-C18 2.7 μm 3.0*50 mm, purity 92.5%, MS ESI calcd. for 474.23 [M+H]⁺ 475.23, found 475.2.

Step 4. To a solution of methyl (1s,4s)-4-(methyl(4-((3-nitro-6-phenylpyridin-2-yl)amino)benzyl)amino)cyclohexane-1-carboxylate (300 mg, 0.630 mmol) in methanol (5 mL), Pd/C (100 mg, 10%) was added under N₂ atmosphere. The suspension was degassed and purged with H₂ (×3) and the reaction mixture was stirred under H₂ (15 psi) at 25° C. for 10 hrs. The mixture was filtered via a celite pad. The pad was washed with (DCM:EtOAc=1:1, 100 mL). The filtrate was concentrated to give methyl (1s,4s)-4-((4-((3-amino-6-phenylpyridin-2-yl)amino)benzyl)(methyl)amino)cyclohexane-1-carboxylate (270 mg, 96.0% yield) as a yellow oil, which was used in next step without further purification. LCMS Rₜ=1.077 min in 2 min chromatography, Agilent PoroShell 120 EC-C18 2.7 μm 3.0*50 mm, purity 86.3%, MS ESI calcd. for 444.25 [M+H]⁺ 445.25, found 445.2.

Step 5. To a solution of methyl (1s,4s)-4-((4-((3-amino-6-phenylpyridin-2-yl)amino)benzyl)(methyl)amino)cyclohexane-1-carboxylate (270 mg, 0.57 mmol) in acetic acid (4 mL), 2-aminopyridine-3-carbaldehyde (104 mg, 0.85 mmol) was added at 25° C. The reaction mixture was degassed and purged with O₂ (×3) and the mixture was stirred at 80° C. for 1 hr. The reaction mixture was concentrated directly to give methyl (1s,4s)-4-((4-(2-(2-aminopyridin-3-yl)-5-phenyl-3H-imidazo[4,5-b]pyridin-3-yl)benzyl)methyl)amino)cyclohexane-1-carboxylate (570 mg, crude) as a yellow oil, which was used in the next step directly. LCMS Rₜ=0.577 min in 1.5 min chromatography, Agilent PoroShell 120 EC-C18 2.7 μm 3.0*50 mm, purity 90.2%, MS ESI calcd. for 546.27 [M+H]⁺ 547.27, found 547.3.

Step 6. To a solution of methyl (1s,4s)-4-((4-(2-(2-aminopyridin-3-yl)-5-phenyl-3H-imidazo[4,5-b]pyridin-3-yl)benzyl)(methyl)amino)cyclohexane-1-carboxylate (520 mg, 0.95 mmol) in THF (3 mL), water (3 mL) and methanol (1 mL), LiOH·H₂O (120 mg, 2.85 mmol) was added and the mixture was stirred at 25° C. for 10 hrs. The residue was purified by prep-HPLC (Column: Welch Xtimate C₁₈ 40*200 mm 7 μm; mobile phase: [water (NH₄HCO₃)-ACN]; B %: 6%-46%; 25 min) to give (1s,4s)-4-((4-(2-(2-aminopyridin-3-yl)-5-phenyl-3H-imidazo[4,5-b]pyridin-3-yl)benzyl)(methyl)amino)cyclohexane-1-carboxylic acid I-95 (87.0 mg, 17.2% yield) as a white solid. ¹H NMR (DMSO-d₆, 400 MHz) δ_{H}=8.26 (d, J=8.4 Hz, 1H), 8.06-7.95 (m, 4H), 7.51-7.35 (m, 7H), 7.19-7.13 (m, 1H), 7.03 (s, 2H), 6.37 (dd, J=4.8, 7.6 Hz, 1H), 3.61 (s, 2H), 2.46-2.37 (m, 2H), 2.11 (s, 3H), 2.07-2.00 (m, 2H), 1.70-1.39 (m, 6H). HPLC Rₜ=1.769 min in 8 min chromatography, 10-80AB, purity 99.7%. LCMS Rₜ=1.307 min in 4 min chromatography, Agilent PoroShell 120 EC-C18 2.7 μm 3.0*50 mm, purity 99.8%, MS ESI calcd. for 532.26 [M+H]⁺ 533.26, found 533.3.

### Example 87. Synthesis of (1r,4r)-4-((4-(2-(2-aminopyridin-3-yl)-5-phenyl-3H-imidazo[4,5-b]pyridin-3-yl)benzyl)(methyl)amino)cyclohexane-1-carboxylic acid (Compound I-96)

### Synthesis of methyl (1r,4r)-4-(methylamino)cyclohexane-1-carboxylate

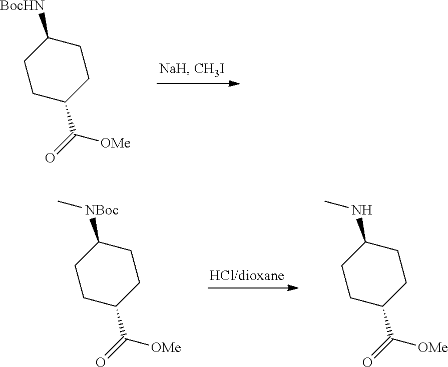

Step 1. To a solution of methyl (1r,4r)-4-((tert-butoxycarbonyl)amino)cyclohexane-1-carboxylate (2.00 g, 7.77 mmol) in DMF (10 mL), NaH (0.62 g, 15.5 mmol) was added portionwise at 0° C. under N₂ atmosphere. After addition, the mixture was stirred at 0° C. for 0.5 hr. Mel (0.97 mL, 15.5 mmol) was then added dropwise at 0° C. under N₂ atmosphere and the resulting mixture was stirred at 25° C. for 16 hrs. The reaction was quenched with aq. NH₄Cl (10 mL), dropwise, at 0° C. under N₂ atmosphere. The resulting mixture was extracted with EtOAc (50 mL×3). The combined organic phase was washed with brine (50 mL), water (50 mL), dried over anhydrous Na₂SO₄, filtered and concentrated to give methyl (1r,4r)-4-((tert-butoxycarbonyl)(methyl)amino)cyclohexane-1-carboxylate (600 mg, 28.5% yield) as a colorless oil. ¹H NMR (400 MHz, DMSO-d₆) δ=3.58 (s, 3H), 2.64 (s, 3H), 2.25 (t, J=12.0, 3.6 Hz, 1H), 1.99-1.90 (m, 2H), 1.61-1.30 (m, 16H).

Step 2. To a solution of trans-methyl 4-[tert-butoxycarbonyl(methyl)amino]cyclohexanecarboxylate (600 mg, 2.21 mmol) in 1,4-dioxane (1 mL), HCl/dioxane (5.0 mL, 2.21 mmol) was added at 25° C. and the mixture was stirred for 2 hrs. The reaction mixture was concentrated directly to give methyl (1r,4r)-4-(methylamino)cyclohexane-1-carboxylate (250 mg, 66.0° % yield) as a white solid, which was used in the next step directly. ¹H NMR (400 MHz, DMSO-d₆) δ=3.59 (s, 3H), 2.90 (s, 1H), 2.49-2.46 (m, 3H), 2.35-2.20 (m, 1H), 2.14-1.91 (m, 4H), 1.39-1.32 (m, 4H).

### Synthesis of (1r,4r)-4-((4-(2-(2-aminopyridin-3-yl)-5-phenyl-3H-imidazo[4,5-b]pyridin-3-yl)benzyl)(methyl)amino)cyclohexane-1-carboxylic acid (Compound 1-96)

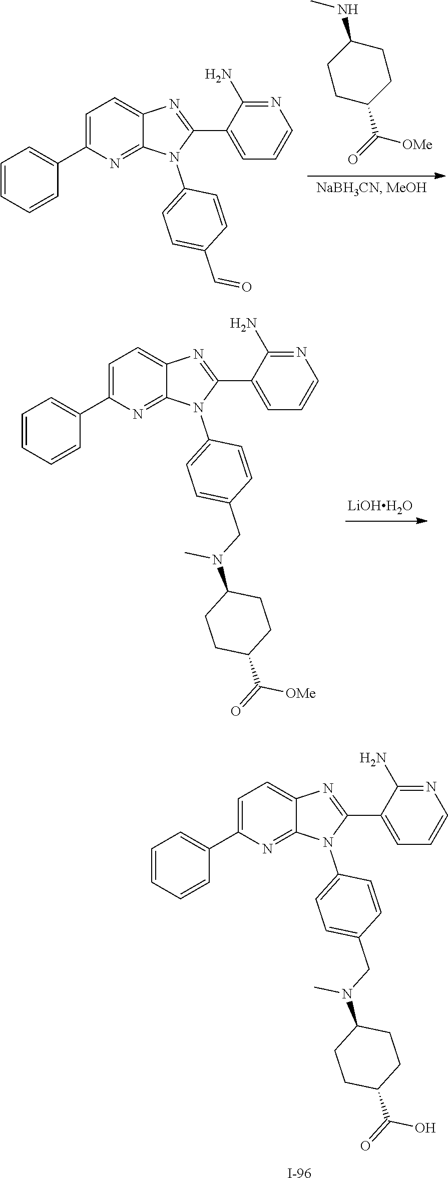

Step 1. To a solution of 4-(2-(2-aminopyridin-3-yl)-5-phenyl-3H-imidazo[4,5-b]pyridin-3-yl)benzaldehyde (150 mg, 0.383 mmol) in MeOH (4 mL), methyl (1r,4r)-4-(methylamino)cyclohexane-1-carboxylate (98 mg, 0.575 mmol), TEA (0.083 mL, 0.575 mmol) and NaBH₃CN (36 mg, 0.575 mmol) were added at 25° C. and the mixture was stirred for 2 hr under N₂ atmosphere. The reaction mixture was concentrated directly. The residue was purified by flash silica gel chromatography (eluent of 0-87% EtOAc in PE) to afford methyl (1r,4r)-4-((4-(2-(2-aminopyridin-3-yl)-5-phenyl-3H-imidazo[4,5-b]pyridin-3-yl)benzyl)(methyl)amino)cyclohexane-1-carboxylate (100 mg, 47.7% yield) as a yellow oil. LCMS Rₜ=0.700 min in 1.5 min chromatography, purity 98.67%, MS ESI calcd. for 546.27 [M+H]⁺ 547.27, found 547.3.

Step 2. To a solution of trans-methyl 4-[[4-[2-(2-amino-3-pyridyl)-5-phenyl-imidazo[4,5-b]pyridin-3-yl]phenyl]methyl-methyl-amino]cyclohexanecarboxylate (80.0 mg, 0.146 mmol) in THF (1 mL) and water (1 mL), LiOH·H₂O (41 mg, 1.00 mmol) was added at 25° C. and the mixture was stirred for 3 hrs. The reaction mixture was then adjusted to pH ~6 by FA. The residue was purified by prep-HPLC (column: Welch Xtimate C18 40*200 mm 7 μm; condition: water(FA)-ACN; begin b: 25-34) to afford (1r,4r)-4-((4-(2-(2-aminopyridin-3-yl)-5-phenyl-3H-imidazo[4,5-b]pyridin-3-yl)benzyl)(methyl)amino)cyclohexane-1-carboxylic acid I-96 (13.0 mg, 15.9% yield) as a light yellow solid. ¹H NMR (400 MHz, DMSO-d₆) δ=12.27-11.74 (m, 1H), 8.27 (d, J=8.4 Hz, 1H), 8.06-7.95 (m, 4H), 7.51-7.36 (m, 7H), 7.18-7.13 (m, 1H), 7.04 (s, 2H), 6.37 (dd, J=7.6, 4.8 Hz, 1H), 3.66 (s, 2H), 2.46-2.39 (m, 1H), 2.21-2.09 (m, 4H), 2.01-1.92 (m, 2H), 1.90-1.81 (m, 2H), 1.43-1.25 (m, 4H). HPLC Rₜ=1.830 min in 8 min chromatography, purity 98.06%. LCMS Rₜ=0.648 min in 1.5 min chromatography, purity 98.57%, MS ESI calcd. For 532.26 [M+H]⁺ 533.26, found 533.3.

### Example 88. Synthesis of 2-(1-(4-(2-(2-aminopyridin-3-yl)-5-phenyl-3H-imidazo[4,5-b]pyridin-3-yl)benzyl)piperidin-4-yl)acetic acid (Compound I-97)

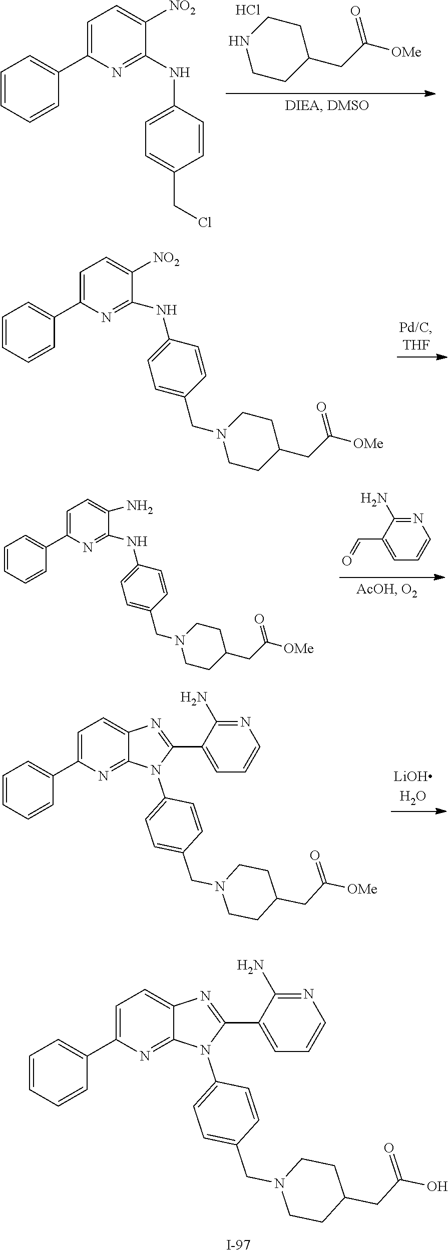

Step 1. To a solution of methyl 2-(piperidin-4-yl)acetate (229 mg, 1.18 mmol) in DMSO (5 mL), DIEA (456 mg, 3.53 mmol) and N-(4-(chloromethyl)phenyl)-3-nitro-6-phenylpyridin-2-amine (200 mg, 0.589 mmol) were added at 25° C. and the mixture was stirred at 80° C. for 14 hrs. EtOAc (30 mL) was added the reaction and the resulting suspension was washed with brine (20 mL×3), dried over anhydrous Na₂SO₄, filtered and concentrated. The residue was purified by flash silica gel chromatography (eluent of 0-43% EtOAc in PE) to give methyl 2-(1-(4-((3-nitro-6-phenylpyridin-2-yl)amino)benzyl)piperidin-4-yl)acetate (350 mg, crude) as a red solid. ¹H NMR (400 MHz, DMSO-d₆) δ5=10.11 (s, IH), 8.61 (d, J=8.8 Hz, 1H), 8.12-8.06 (m, 2H), 7.69 (d, J=8.4 Hz, 2H), 7.59 (d, J=8.8 Hz, 1H), 7.55-7.50 (m, 3H), 7.33 (d, J=8.4 Hz, 2H), 3.58 (s, 3H), 3.45 (s, 2H), 2.84-2.77 (m, 2H), 2.52-2.51 (m, 2H), 2.24 (d, J=6.8 Hz, 2H), 1.97-1.88 (m, 2H), 1.67-1.56 (m, 3H).

Step 2. To a mixture of methyl 2-(1-(4-((3-nitro-6-phenylpyridin-2-yl)amino)benzyl)piperidin-4-yl)acetate (300 mg, 0.651 mmol) in THF (5 mL), Pd/C (150 mg, 10%) was added under an Ar atmosphere and the suspension was degassed and purged with H₂ (×3). The mixture was stirred under H₂ (15 psi) at 25° C. for 1 hr. The mixture was filtered via a celite pad and the pad was washed with THF (40 ml). The filtrate was concentrated to give methyl 2-(1-(4-((3-amino-6-phenylpyridin-2-yl)amino)benzyl)piperidin-4-yl)acetate (300 mg, crude) as a yellow oil. ¹H NMR (400 MHz, DMSO-d₆) δ_{H}=7.91 (d, J=7.6 Hz, 2H), 7.82 (s, IH), 7.72 (d, J=8.4 Hz, 2H), 7.42-7.34 (m, 2H), 7.28-7.16 (m, 4H), 6.97 (d, J=8.0 Hz, 1H), 5.25 (s, 2H), 3.57 (s, 3H), 3.33-3.32 (m, 2H), 2.80 (d, J=11.2 Hz, 2H), 2.52-2.51 (m, 2H), 2.23 (d, J=6.8 Hz, 2H), 1.95-1.85 (m, 2H), 1.69-1.51 (m, 3H).

Step 3. To a solution of methyl 2-(1-(4-((3-amino-6-phenylpyridin-2-yl)amino)benzyl)piperidin-4-yl)acetate (250 mg, 0.581 mmol) in AcOH (5.0 mL, 0.581 mmol), 2-aminonicotinaldehyde (106 mg, 0.871 mmol) was added and the reaction mixture was degassed and purged with 02 (×3). The mixture was stirred at 80° C. for 2 hr under O₂. The reaction mixture was concentrated directly. The reaction mixture was diluted with EtOAc (30 mL), washed with saturated aqueous NaHCO₃ (40 mL×2) and brine (20 mL), dried over anhydrous Na₂SO₄, filtered and concentrated to give methyl 2-(1-(4-(2-(2-aminopyridin-3-yl)-5-phenyl-3H-imidazo[4,5-b]pyridin-3-yl)benzyl)piperidin-4-yl)acetate (300 mg, 97.0% yield) as a yellow oil. LCMS Rₜ=0.479 min in 1 min chromatography, purity 94.21%, MS ESI calcd. for 532.26, [M+H]⁺ 533.26, found 533.3.

Step 4. To a solution of methyl 2-(1-(4-(2-(2-aminopyridin-3-yl)-5-phenyl-3H-imidazo[4,5-b]pyridin-3-yl)benzyl)piperidin-4-yl)acetate (140 mg, 0.263 mmol) in THF (3 mL), water (1.5 mL) and methanol (1.5 mL), LiOH·H₂O (33 mg, 0.789 mmol) was added and the mixture was stirred at 25° C. for 1 hr. The residue was purified by prep-HPLC (Column: Welch Xtimate C18 40*200 mm 7 μm; mobile phase: water (NH₄HCO₃)-ACN: B %: 4%-44%; 25 min) to give 2-(1-(4-(2-(2-aminopyridin-3-yl)-5-phenyl-3H-imidazo[4,5-b]pyridin-3-yl)benzyl)piperidin-4-yl)acetic acid I-97 (27.3 mg, 20.0/6 yield) as a white solid. ¹H NMR (400 MHz, DMSO-d₆) δ=8.27 (d, J=8.4 Hz, 1H), 8.06-7.95 (m, 4H), 7.51-7.38 (m, 7H), 7.15 (d, J=7.6 Hz, 1H), 7.03 (s, 2H), 6.37 (dd, J=4.8, 7.6 Hz, 1H), 3.54 (s, 2H), 2.81 (d, J=11.2 Hz, 2H), 2.14 (d, J=6.4 Hz, 2H), 1.97 (t, J=11.2 Hz, 2H), 1.65 (d, J=10.0 Hz, 3H), 1.29-1.16 (m, 2H). HPLC Rₜ=3.107 min in 8 min chromatography, purity 98.79%. LCMS Rₜ=1.800 min in 4 min chromatography, purity 99.48%, MS ESI calcd. for 518.24, [M+H]⁺519.24, found 519.2.

### Example 89. Synthesis of 2-(1-(4-(2-(2-aminopyridin-3-yl)-5-phenyl-3H-imidazo[4,5-b]pyridin-3-yl)benzyl)piperidin-4-yl)propanoic acid (Compound I-98)

### Synthesis of methyl 2-(4-piperidyl)propanoate

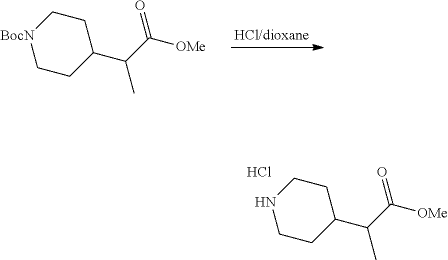 [SMILES: COC(=O)C(C)C1CCN(C(=O)OC(C)(C)C)CC1 ; Cl ; COC(=O)C(C)C1CCNCC1]

Step 1. To a solution of tert-butyl 4-(2-methoxy-1-methyl-2-oxo-ethyl)piperidine-1-carboxylate (400 mg, 1.47 mmol) in 1,4-dioxane (2 mL), HCl/dioxane (6.0 mL, 1.47 mmol) was added under N₂ atmosphere and the mixture was stirred at 20° C. for 6 hrs. The reaction mixture was concentrated to give methyl 2-(4-piperidyl)propanoate (250 mg, 99.0% yield) as a white solid. ¹H NMR (400 MHz, DMSO-d₆) δ=8.80 (d, J=1.6 Hz, 1H), 8.64-8.34 (m, 1H), 3.61 (s, 3H), 3.24 (s, 2H), 2.82 (d, J=12.4 Hz, 2H), 2.34 (t, J=6.8 Hz, 1H), 1.84-1.71 (m, 2H), 1.64 (d, J=13.6 Hz, 1H), 1.47-1.31 (m, 2H), 1.05 (d, J=7.2 Hz, 3H).

### Synthesis of 2-(1-(4-(2-(2-aminopyridin-3-yl)-5-phenyl-3H-imidazo[4,5-b]pyridin-3-yl)benzyl)piperidin-4-yl)propanoic acid (Compound I-98)

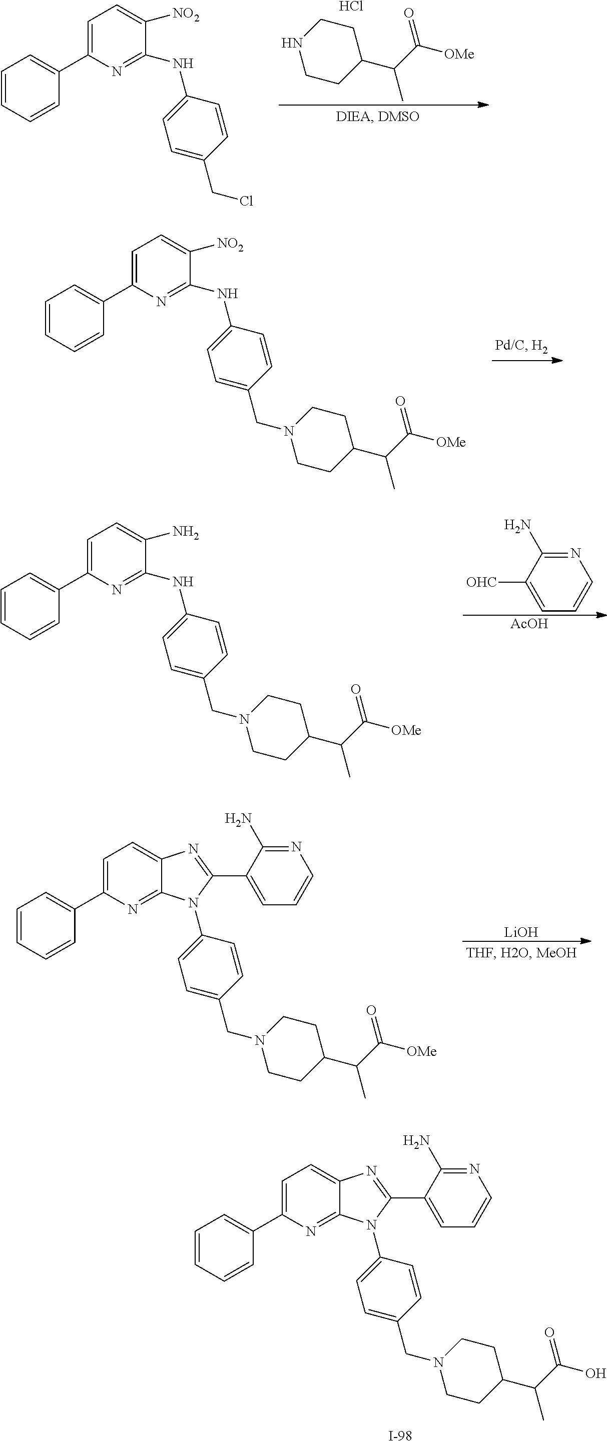

Step 1. To a solution of methyl 2-(4-piperidyl)propanoate;hydrochloride (208 mg, 1.00 mmol) in DMSO (4 mL), N-[4-(chloromethyl)phenyl]-3-nitro-6-phenyl-pyridin-2-amine (200 mg, 0.589 mmol) and DIEA (0.58 mL, 3.53 mmol) were added under N₂ atmosphere and the mixture was stirred at 80° C. for 6 hrs. The reaction was taken up in EtOAc (30 mL) and the organics were washed with brine solution (3×50 mL). The organics were then separated and dried (Na₂SO₄) before concentration to give methyl 2-[1-[[4-[(3-nitro-6-phenyl-2-pyridyl)amino]phenyl]methyl]-4-piperidyl]propanoate (0.600 g, crude) as a brown oil. ¹H NMR (400 MHz, DMSO-d₆) δ=10.15 (s, 1H), 8.65 (d, J=8.8 Hz, 1H), 8.17-8.10 (m, 2H), 7.73 (d, J=8.4 Hz, 2H), 7.63 (d, J=8.4 Hz, 1H), 7.60-7.55 (m, 3H), 7.37 (d, J=8.4 Hz, 2H), 3.63 (s, 3H), 3.49 (s, 2H), 2.93-2.84 (m, 2H), 2.33 (br t, J=7.2 Hz, 1H), 1.98-1.87 (m, 2H), 1.69-1.59 (m, 1H), 1.56-1.43 (m, 2H), 1.33-1.25 (m, 2H), 1.09 (d, J=6.8 Hz, 3H).

Step 2. To a solution of methyl 2-[1-[[4-[(3-nitro-6-phenyl-2-pyridyl)amino]phenyl]methyl]-4-piperidyl]propanoate (600 mg, 1.26 mmol) in methanol (5 mL), wet Pd/C (200 mg) was added under N₂ atmosphere. The suspension was degassed and purged with H₂ (×3). The reaction mixture was stirred under O₂ (15 psi) at 40° C. for 16 hrs. After cooling to room temperature, the mixture was filtered through celite. The filtrate was concentrated to obtain methyl 2-[1-[[4-[(3-amino-6-phenyl-2-pyridyl)amino]phenyl]methyl]-4-piperidyl]propanoate (430 mg, crude) as a brown oil. LCMS Rₜ=0.507 min in 1 min chromatography, Agilent PoroShell 120 EC-C18 2.7 um 3.0*50 mm, purity 86.6, MS ESI calcd. for 444.25 [M+H]+ 445.25, found 445.3.

Step 3. A solution of methyl 2-[1-[[4-[(3-amino-6-phenyl-2-pyridyl)amino]phenyl]methyl]-4-piperidyl]propanoate (350 mg, 0.787 mmol), 2-aminopyridine-3-carbaldehyde (144 mg, 1.18 mmol) in acetic acid (3 mL) under O₂ was stirred at 80° C. for 1 hr. The reaction mixture was concentrated to give methyl 2-[1-[[4-[2-(2-amino-3-pyridyl)-5-phenyl-imidazo[4,5-b]pyridin-3-yl]phenyl]methyl]-4-piperidyl]propanoate (300 mg, 69.7% yield) as a yellow oil. LCMS Rₜ=0.457 min in 1 min chromatography, Agilent PoroShell 120 EC-C18 2.7 um 3.0*50 mm, purity 86.62%, MS ESI calcd. for 546.27 [M+H]+ 547.27, found 547.2.

Step 4. To a solution of methyl 2-[1-[[4-[2-(2-amino-3-pyridyl)-5-phenyl-imidazo[4,5-b]pyridin-3-yl]phenyl]methyl]-4-piperidyl]propanoate (300 mg, 0.732 mmol) in THF (2 mL), water (1 mL) and methanol (I mL), LiOH·H₂O (184 mg, 4.39 mmol) was added and the mixture was stirred at 25° C. for 1 hr. The crude product was purified by prep-HPLC (column: Welch Xtimate C18 200*40 mm*7 μm; mobile phase: [water(NH₄HCO₃)-ACN];B %: 6%-46%, 25 min) to give 2-[1-[[4-[2-(2-amino-3-pyridyl)-5-phenyl-imidazo[4,5-b]pyridin-3-yl]phenyl]methyl]-piperidyl]propanoic acid I-98 (65.0 mg, 16.5% yield) as a white solid. ¹H NMR (400 MHz, DMSO-d₆) δ=8.26 (d, J=8.4 Hz, 1H), 8.04-7.96 (m, 4H), 7.48-7.37 (m, 7H), 7.15 (d, J=7.6 Hz, 1H), 7.02 (s, 2H), 6.37 (dd, J=4.8, 7.2 Hz, 1H), 3.53 (s, 2H), 2.88-2.82 (m, 2H), 2.11 (t, J=6.8 Hz, 1H), 1.93 (t, J=11.2 Hz, 2H), 1.64-1.52 (m, 2H), 1.48-1.39 (m, 1H), 1.30-1.18 (m, 21), 1.00 (d, J=6.8 Hz, 31H). HPLC Rₜ=1.843 min in 8 min chromatography, ACE Excel 3 C18 4.6*100 mm, purity 99.0%. LCMS Rₜ=1.450 min in 4 min chromatography. Agilent PoroShell 120 EC-C18 2.7 um 3.0*50 mm, purity 96.501%, MS ESI calcd. for 532.26 [M+H]⁺ 533.26, found 533.3.

### Example 90. Synthesis of 1-(4-(2-(2-aminopyridin-3-yl)-5-phenyl-3H-imidazo[4,5-b]pyridin-3-yl)benzyl)-2,2-dimethylpiperidine-4-carboxylic acid (Compound I-99)

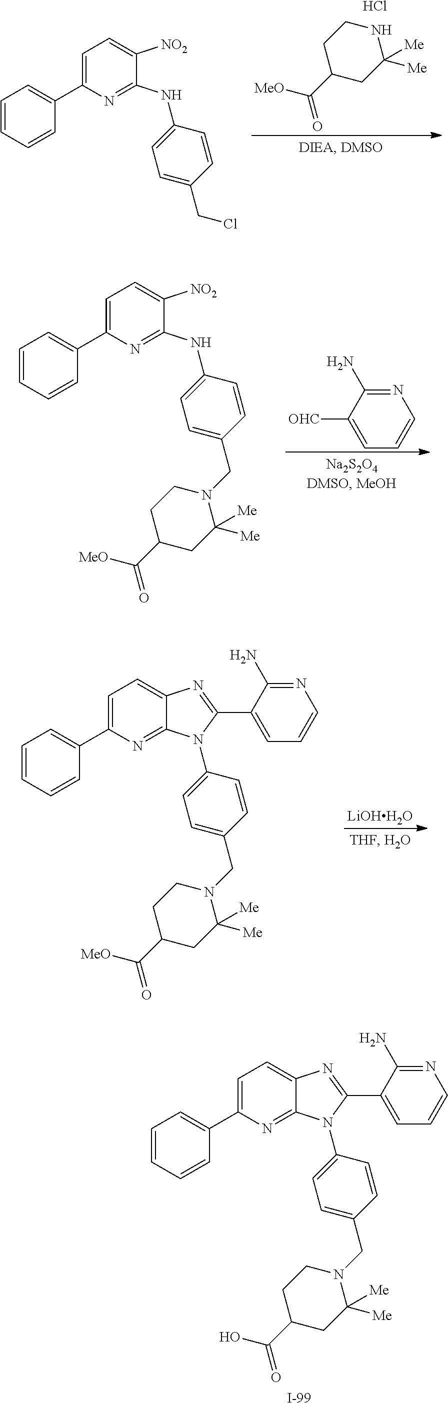

Step 1. To a solution of N-(4-(chloromethyl)phenyl)-3-nitro-6-phenylpyridin-2-amine (300 mg, 0.883 mmol) in DMSO (4 mL), DIEA (685 mg, 5.30 mmol) and methyl 2,2-dimethylpiperidine-4-carboxylate; hydrochloride (275 mg, 1.32 mmol) were added at 25° C. and the mixture was stirred at 80° C. for 14 hrs. EtOAc (30 mL) was added to the reaction and the resulting suspension was washed with brine (20 mL×3), dried over anhydrous Na₂SO₄, filtered and concentrated. The residue was purified by flash silica gel chromatography (eluent of 0-18% EtOAc in PE) to give methyl 2,2-dimethyl-1-[[4-[(3-nitro-6-phenyl-2-pyridyl)amino]phenyl]methyl]piperidine-4-carboxylate (270 mg, 64.4% yield) as a yellow oil. ¹H NMR (400 MHz, DMSO-d₆) δ=10.11 (s, 1H), 8.61 (d, J=8.4 Hz, 1H), 8.15-8.03 (m, 2H), 7.68 (d, J=8.4 Hz, 2H), 7.61-7.51 (m, 4H), 7.37 (d, J=8.0 Hz, 2H), 4.06-3.96 (m, 2H), 3.60 (s, 3H), 2.98 (d, J=14.4 Hz, 1H), 2.31-2.22 (m, 1H), 1.77-1.65 (m, 2H), 1.57-1.47 (m, 1H), 1.44-1.35 (m, 1H), 1.25 (s, 31H), 1.21-1.14 (nm, 1H), 1.04 (s, 3H).

Step 2. To a solution of methyl 2,2-dimethyl-1-[[4-[(3-nitro-6-phenyl-2-pyridyl)amino]phenyl]methyl]piperidine-4-carboxylate (200 mg, 0.421 mmol) in DMSO (4 mL) and methanol (2 mL), 2-aminopyridine-3-carbaldehyde (154 mg, 1.26 mmol) and Na₂S₂O₄ (0.44 g, 2.53 mmol) were added at 25° C. and the mixture was stirred at 100° C. for 10 hrs. Water (5 mL) was added and the mixture was extracted with EtOAc (15 mL×3). The organic phase was washed with brine (5 mL), dried over Na₂SO₄, filtered, and concentrated under reduced pressure to provide the crude material. The residue was purified by flash silica gel chromatography (eluent of 0-7% MeOH in DCM) to give methyl 1-[[4-[2-(2-amino-3-pyridyl)-5-phenyl-imidazo[4,5-b]pyridin-3-yl]phenyl]methyl]-2,2-dimethyl-piperidine-4-carboxylate (40.0 mg, 17.4% yield) as a yellow oil. LCMS Rₜ=0.477 min in 1.0 min chromatography, purity 92.1%, MS ESI calcd. for 546.27 [M+H]⁺ 547.27, found 547.4.

Step 3. To a solution of methyl 1-[[4-[2-(2-amino-3-pyridyl)-5-phenyl-imidazo[4,5-b]pyridin-3-yl]phenyl]methyl]-2,2-dimethyl-piperidine-4-carboxylate (35 mg, 0.0640 mmol) in THF (1 mL), LiOH·H₂O (8.1 mg, 0.192 mmol) and water (0.5 mL) were added and the mixture was stirred at 25° C. for 2 hrs. The reaction mixture was filtered, and the filtrate was concentrated to dryness. The residue was purified by prep-HPLC (Column: Welch Xtimate C18 40*200 mm 7 μm; mobile phase: [water (NH₄HCO₃)-ACN], B %: 8%-48%, 25 min) to provide 1-(4-(2-(2-aminopyridin-3-yl)-5-phenyl-3H-imidazo[4,5-b]pyridin-3-yl)benzyl)-2,2-dimethylpiperidine-4-carboxylic acid 1-99 (10.9 mg, 27.1% yield) as a white solid. ¹H NMR (400 MHz, DMSO-d₆) δ=8.26 (d, J=8.4 Hz, 1H), 8.06-7.92 (m, 4H), 7.54-7.35 (m, 7H), 7.16 (dd, J=1.6, 7.6 Hz, 1H), 7.01 (s, 2H), 6.37 (dd, J=4.8, 7.6 Hz, 1H), 4.08 (d, J=14.4 Hz, 1H), 3.07 (d, J=14.4 Hz, 1H), 2.47-2.39 (m, 2H), 2.34-2.29 (m, 1H), 1.79-1.64 (m, 2H), 1.54-1.34 (m, 2H), 1.23 (s, 3H), 1.03 (s, 3H).

### Example 91. Synthesis of 6-((4-(2-(2-aminopyridin-3-yl)-5-phenyl-3H-imidazo[4,5-b]pyridin-3-yl)-3-methylphenyl)carbamoyl)spiro[3.3]heptane-2-carboxylic acid (Compound I-100)

### Synthesis of tert-butyl (4-amino-3-methylphenyl)carbamate

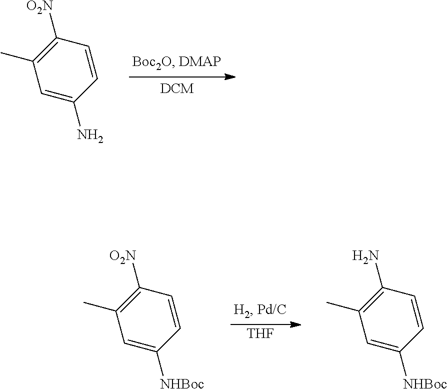 [SMILES: Cc1cc(N)ccc1[N+](=O)[O-] ; Cc1cc(NC(=O)OC(C)(C)C)ccc1[N+](=O)[O-] ; Cc1cc(NC(=O)OC(C)(C)C)ccc1N ; "THF"]

Step 1. To a solution of 3-methyl-4-nitroaniline (2.50 g, 16.4 mmol) and Boc₂O (4.30 g, 19.7 mmol) in DCM (20 mL), DMAP (0.40 g, 3.29 mmol) was added and the mixture was stirred at 25° C. for 16 hrs. Water (10 mL) was added to the residue and the resulting mixture was extracted with DCM (10 mL×3). The combined organic phase was washed with brine (10 mL), dried over anhydrous Na₂SO₄, filtered and concentrated. The residue was purified by flash silica gel chromatography (eluent of 0-5% EtOAc in PE) to give tert-butyl (3-methyl-4-nitrophenyl)carbamate (2.50 g, 42.2% yield) as a light yellow oil. ¹H NMR (DMSO-d₆, 400 MHz) δ_{H}=9.93 (s, 1H), 7.99 (d, J=9.2 Hz, 1H), 7.53 (d, J=2.0 Hz, 1H), 7.49-7.44 (m, 1H), 1.48 (s, 9H), 1.44-1.37 (m, 3H).

Step 2. To a solution of tert-butyl (3-methyl-4-nitrophenyl)carbamate (1.60 g, 6.34 mmol) in THF (20 mL), Pd/C (500 mg, purity 10%) was added under N₂. The reaction mixture was degassed and purged with H₂ (30 psi) (×3) and the mixture was stirred at 25° C. for 12 hrs under H₂ (30 psi). The reaction mixture was filtered and the filtrate was concentrated directly to give tert-butyl (4-amino-3-methylphenyl)carbamate (1.40 g, 99.3% yield) as a light yellow oil, which was used in the next step directly. ¹H NMR (DMSO-d₆, 400 MHz) δ_{H}=6.73-6.68 (m, 2H), 6.57-6.53 (m, 1H), 4.94 (s, 2H), 2.02 (s, 3H), 1.38 (s, 9H).

### Synthesis of 6-((4-(2-(2-aminopyridin-3-yl)-5-phenyl-3H-imidazo[4,5-b]pyridin-3-yl)-3-methylphenyl)carbamoyl)spiro[3.3]heptane-2-carboxylic acid (Compound I-100)

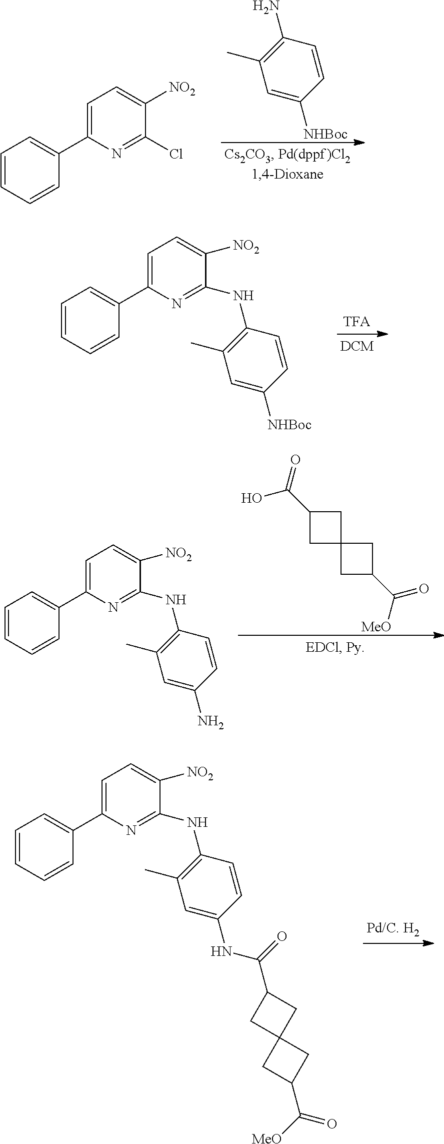

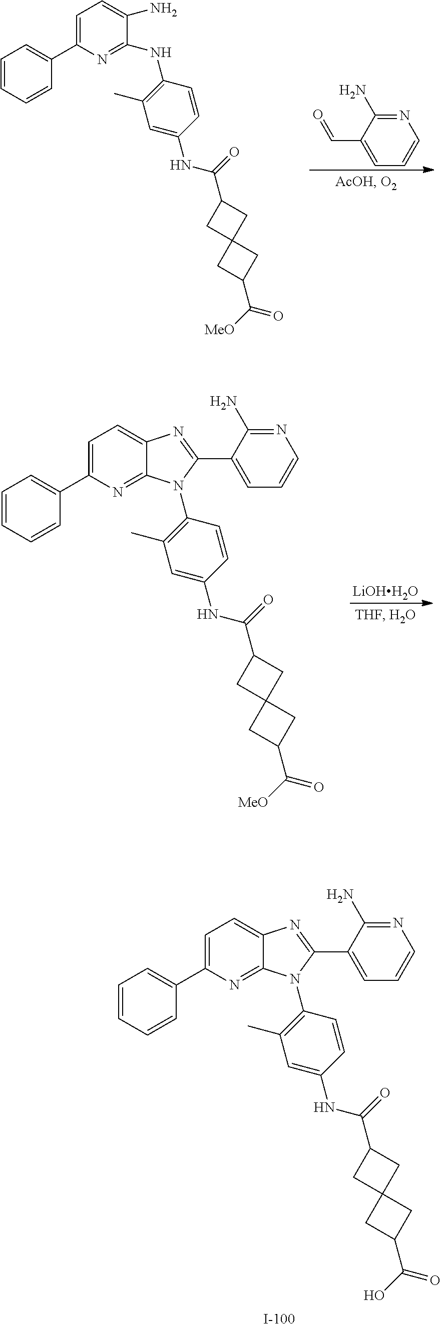 [SMILES: COC(=O)C1CC2(CC(C(=O)Nc3ccc(Nc4nc(-c5ccccc5)ccc4N)c(C)c3)C2)C1 ; Nc1ncccc1C=O ; COC(=O)C1CC2(CC(C(=O)Nc3ccc(-n4c(-c5cccnc5N)nc5ccc(-c6ccccc6)nc54)c(C)c3)C2)C1 ; "LiOH·H2O" ; Cc1cc(NC(=O)C2CC3(CC(C(=O)O)C3)C2)ccc1-n1c(-c2cccnc2N)nc2ccc(-c3ccccc3)nc21]

Step 1. To a solution of 2-chloro-3-nitro-6-phenylpyridine (1.50 g, 6.39 mmol) and tert-butyl (4-amino-3-methylphenyl)carbamate (2.13 g, 9.59 mmol) in 1,4-dioxane (20 mL), Cs₂CO₃ (6.25 g, 19.2 mmol) and Pd(dppf)Cl₂ (468 mg, 0.639 mmol) were added. The reaction mixture was degassed and purged with N₂ (×3) and the mixture was stirred at 80° C. for 12 hrs under N₂. Water (15 mL) was added to the residue and the resulting mixture was extracted with EtOAc (20 mL×3). The combined organic phase was washed with brine (20 mL), dried over anhydrous Na₂SO₄, filtered and concentrated. The residue was purified by flash silica gel chromatography (eluent of 0-10% EtOAc in PE) to give tert-butyl (3-methyl-4-((3-nitro-6-phenylpyridin-2-yl)amino)phenyl)carbamate (1.30 g, 48.4% yield) as a yellow oil. ¹H NMR (DMSO-d₆, 400 MHz) δ_{H}=9.90 (s, 1H), 9.34 (s, 1H), 8.59 (d, J=8.8 Hz, 1H), 8.01-7.96 (m, 2H), 7.63 (d, J=8.8 Hz, 1H), 7.54-7.47 (m, 4H), 7.44 (s, 1H), 7.36 (dd, J=2.0, 8.8 Hz, 1H), 2.22 (s, 3H), 1.49 (s, 9H).

Step 2. To a solution tert-butyl (3-methyl-4-((3-nitro-6-phenylpyridin-2-yl)amino)phenyl)carbamate (800 mg, 1.90 mmol) in DCM (10 mL), TFA (217 mg, 1.90 mmol) was added at 25° C. and the mixture was stirred for 12 hrs. The reaction mixture was concentrated directly to give 2-methyl-N1-(3-nitro-6-phenylpyridin-2-yl)benzene-1,4-diamine (600 mg, 91.6% yield) as a yellow oil, which was used in the next step directly. LCMS Rₜ=0.568 min in 1 min chromatography, purity 86.4%, MS ESI calcd. for 320.35 [M+H]⁺ 321.35, found 321.2.

Step 3. To a solution of 2-methyl-N1-(3-nitro-6-phenylpyridin-2-yl)benzene-1,4-diamine (300 mg, 0.936 mmol) and 6-(methoxycarbonyl)spiro[3.3]heptane-2-carboxylic acid (186 mg, 0.936 mmol) in pyridine (8 mL), EDCI (269 mg, 1.40 mmol) was added and the mixture was stirred at 25° C. for 2 hrs. Water (5 mL) was added to the residue and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent of 10-20% EtOAc in PE) to give methyl 6-((3-methyl-4-((3-nitro-6-phenylpyridin-2-yl)amino)phenyl)carbamoyl)spiro[3.3]heptane-2-carboxylate (100 mg, 21.3% yield) as a red solid. ¹H NMR (DMSO-d₆, 400 MHz) δ_{H}=9.92 (s, 1H), 9.77 (s, 1H), 8.59 (d, J=8.8 Hz, 1H), 8.00-7.97 (m, 2H), 7.68 (d, J=8.8 Hz, 1H), 7.59-7.52 (m, 3H), 7.51-7.48 (m, 3H), 3.59 (s, 3H), 2.52 (s, 2H), 2.34-2.25 (m, 4H), 2.24 (s, 3H), 2.19-2.09 (m, 4H).

Step 4. To a solution of methyl 6-((3-methyl-4-((3-nitro-6-phenylpyridin-2-yl)amino)phenyl)carbamoyl)spiro[3.3]heptane-2-carboxylate (120 mg, 0.240 mmol) in THE (10 mL), Pd/C (50 mg, purity 10%) was added under N₂. The reaction mixture was degassed and purged with H₂ (15 psi) (×3) and the mixture was stirred at 25° C. for 12 hrs under H₂ (15 psi). The reaction mixture was filtered and the filtrate was concentrated directly to give methyl 6-((4-((3-amino-6-phenylpyridin-2-yl)amino)-3-methylphenyl)carbamoyl)spiro[3.3]heptane-2-carboxylate (100 mg, 88.6% yield) as a light yellow oil, which was used in the next step directly. LCMS Rₜ=0.590 min in 1 min chromatography, purity 88.9%, MS ESI calcd. for 470.23, [M+H]⁺ 471.23, found 471.3.

Step 5. To a solution of methyl 6-((4-((3-amino-6-phenylpyridin-2-yl)amino)-3-methylphenyl)carbamoyl)spiro[3.3]heptane-2-carboxylate (100 mg, 0.213 mmol) in acetic acid (3 mL), 2-aminonicotinaldehyde (31 mg, 0.255 mmol) was added under 02. The reaction mixture was degassed and purged with O₂ (×3) and the mixture was stirred at 80° C. for 1 hr under O₂. The reaction mixture was concentrated directly to provide methyl 6-((4-(2-(2-aminopyridin-3-yl)-5-phenyl-3H-imidazo[4,5-b]pyridin-3-yl)-3-methylphenyl)carbamoyl)spiro[3.3]heptane-2-carboxylate (240 mg, 97.9% yield) as a light yellow oil, which was used in the next step directly. LCMS Rₜ=0.505 min in 1 min chromatography, purity 83.6%, MS ESI calcd for 572.25 [M+H]⁺ 573.25, found 573.3.

Step 6. To a solution of methyl 6-((4-(2-(2-aminopyridin-3-yl)-5-phenyl-3H-imidazo[4,5-b]pyridin-3-yl)-3-methylphenyl)carbamoyl)spiro[3.3]heptane-2-carboxylate (100 mg, 0.175 mmol) in THF (3 mL) and water (3 mL), LiOH·H₂O (15 mg, 0.349 mmol) was added at 25° C. and the mixture was stirred for 12 hrs. The mixture was filtered directly. The residue was purified by prep-HPLC (Column: Welch Xtimate C18 150*30 mm*5 μm; mobile phase: [water(NH₄HCO₃)-ACN]; B %: 8%-48%; 25 min) to afford 6-((4-(2-(2-aminopyridin-3-yl)-5-phenyl-3H-imidazo[4,5-b]pyridin-3-yl)-3-methylphenyl)carbamoyl)spiro[3.3]heptane-2-carboxylic acid 1-100 (64.6 mg, 65.8% yield) as a light yellow solid. ¹H NMR (DMSO-d₆, 400 MHz) δ_{H}=9.97 (s, 1H), 8.27 (d, J=8.4 Hz, 1H), 8.02-7.94 (m, 4H), 7.73 (s, 1H), 7.58 (d, J=8.4 Hz, 1H), 7.48-7.42 (m, 2H), 7.41-7.28 (m, 4H), 7.10 (dd, J=1.6, 7.6 Hz, 1H), 6.38 (dd, J=4.8, 7.6 Hz, 1H), 3.15-3.09 (m, 1H), 2.96-2.87 (m, 1H), 2.30-2.20 (m, 4H), 2.19-2.07 (m, 4H), 1.92 (s, 3H).

### Example 92. Synthesis of 6-((4-(2-(2-aminopyridin-3-yl)-5-phenyl-3H-imidazo[4,5-b]pyridin-3-yl)-2-fluoro-3-methylphenyl)carbamoyl)spiro[3.3]heptane-2-carboxylic acid (Compound I-101)

### Synthesis of tert-butyl (4-amino-2-fluoro-3-methylphenyl)carbamate

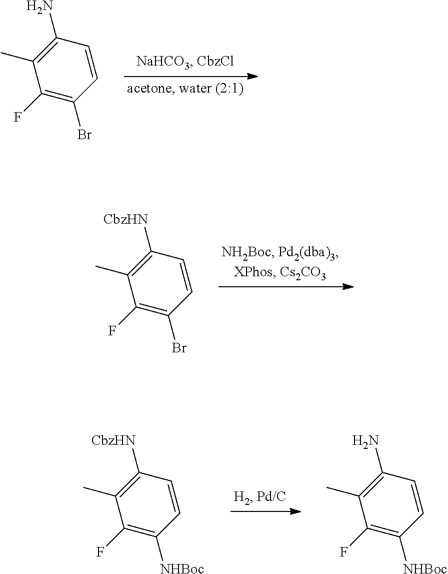

Step 1. To a solution of 4-bromo-3-fluoro-2-methylaniline (3.42 g, 16.8 mmol) in acetone (30 mL) and water (15 mL), NaHCO₃ (2.82 g, 33.5 mmol) and CbzCl (2.70 mL, 20.1 mmol) was added at 0° C. and the mixture was stirred at 25° C. for 24 hrs. The reaction mixture was concentrated. Water (20 mL) was added to the residue and the resulting mixture was extracted with EtOAc (10 mL×3). The combined organic phase was washed with brine (10 mL), water (10 mL), dried over anhydrous Na₂SO₄, filtered and concentrated. The residue was purified by flash silica gel chromatography (eluent of 0-7% EtOAc in PE) to give benzyl (4-bromo-3-fluoro-2-methylphenyl)carbamate (5.28 g, 93.2% yield) as a yellow solid. ¹H NMR (400 MHz, DMSO-d₆) δ_{H}=9.30 (s, 1H), 7.48 (t, J=8.4 Hz, 1H), 7.44-7.29 (m, 6H), 5.15 (s, 2H), 2.14 (d, J=2.4 Hz, 3H).

Step 2. To a solution of benzyl (4-bromo-3-fluoro-2-methylphenyl)carbamate (2.35 g, 6.95 mmol) in toluene (25 mL), NH₂Boc (1.22 g, 10.4 mmol), Cs₂CO₃ (4.53 g, 13.9 mmol), XPhos (663 mg, 1.39 mmol) and Pd₂(dba)₃ (636 mg, 0.695 mmol) were added at 25° C. and the mixture was stirred at 100° C. for 16 hrs. Water (100 mL) was added to the reaction mixture and the whole was extracted with EtOAc (50 mL×3). The combined organic phase was washed with brine (100 mL), dried over anhydrous Na₂SO₄, filtered and concentrated. The residue was purified by flash silica gel chromatography (eluent of 0-9% EtOAc in PE) to give benzyl tert-butyl (2-fluoro-3-methyl-1,4-phenylene)dicarbamate (550 mg, 21.14% yield) as a light yellow solid. ¹H NMR (400 MHz, DMSO-d₆) δ_{H}=9.10 (s, 1H), 8.82 (s, 1H), 7.42-7.29 (m, 6H), 7.09 (d, J=8.8 Hz, 1H), 5.12 (s, 2H), 2.07 (d, J=2.0 Hz, 3H), 1.44 (s, 9H).

Step 3. To a solution of benzyl tert-butyl (2-fluoro-3-methyl-1,4-phenylene)dicarbamate (750 mg, 2.00 mmol) in methanol (15 mL), wet Pd/C (10%, 0.4 g) was added under N₂ atmosphere. The suspension was degassed and purged with H₂ (×3) and the reaction mixture was stirred under H₂ (15 psi) at 25° C. for 16 hrs. The reaction mixture was filtered. The filter cake was washed with MeOH (20 mL). The filtrate was concentrated to give tert-butyl N-(4-amino-2-fluoro-3-methyl-phenyl)carbamate (400 mg, 83.1% yield) as a purple oil, which was used in the next step directly.

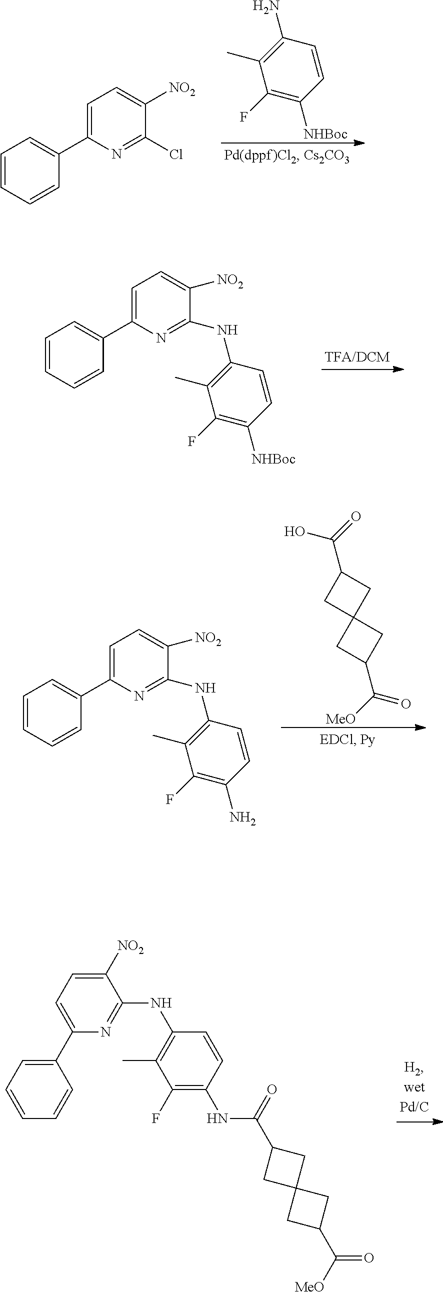 [SMILES: O=[N+]([O-])c1ccc(-c2ccccc2)nc1Cl ; Cc1c(N)ccc(NC(=O)OC(C)(C)C)c1F ; Cc1c(Nc2nc(-c3ccccc3)ccc2[N+](=O)[O-])ccc(NC(=O)OC(C)(C)C)c1F ; Cc1c(Nc2nc(-c3ccccc3)ccc2[N+](=O)[O-])ccc(N)c1F ; COC(=O)C1CC2(CC(C(=O)O)C2)C1 ; COC(=O)C1CC2(CC(C(=O)Nc3ccc(Nc4nc(-c5ccccc5)ccc4[N+](=O)[O-])c(C)c3F)C2)C1]

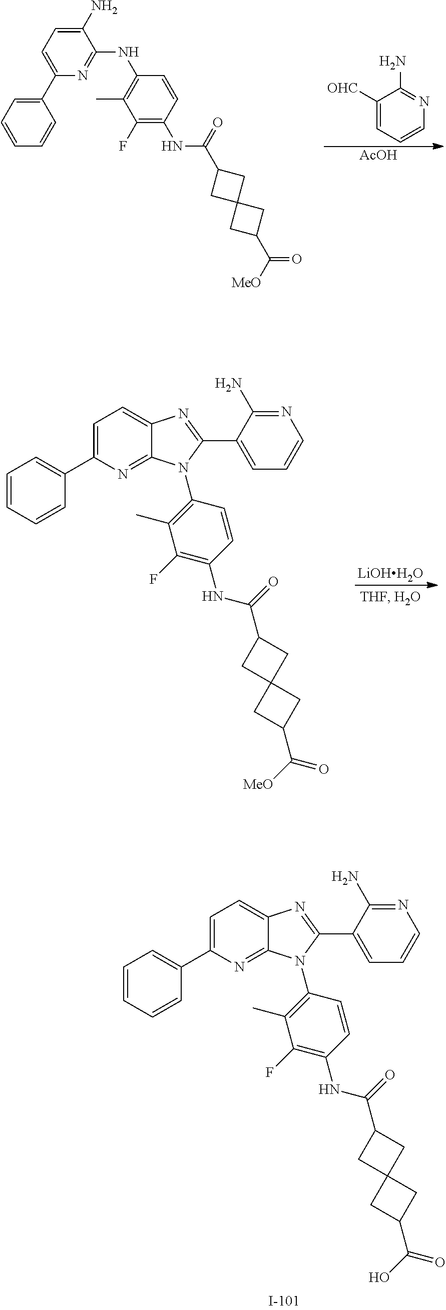 [SMILES: "AcOH" ; COC(=O)C1CC2(CC(C(=O)Nc3ccc(Nc4nc(-c5ccccc5)ccc4N)c(C)c3F)C2)C1 ; Nc1ncccc1C=O ; COC(=O)C1CC2(CC(C(=O)Nc3ccc(-n4c(-c5cccnc5N)nc5ccc(-c6ccccc6)nc54)c(C)c3F)C2)C1 ; "LiOH·H2O" ; Cc1c(-n2c(-c3cccnc3N)nc3ccc(-c4ccccc4)nc32)ccc(NC(=O)C2CC3(CC(C(=O)O)C3)C2)c1F]

Step 1. To a solution of 2-chloro-3-nitro-6-phenylpyridine (289 mg, 1.23 mmol) and tert-butyl (4-amino-2-fluoro-3-methylphenyl)carbamate (400 mg, 1.66 mmol) in 1,4-dioxane (7 mL), Cs₂CO₃ (1.21 g, 3.70 mmol) and Pd(dppf)Cl₂ (90 mg, 0.123 mmol) was added. The reaction mixture was degassed and purged with N₂ (×3) and the mixture was stirred at 80° C. for 12 hrs under N₂. Water (30 mL) was added to the residue and the resulting mixture was extracted with EtOAc (20 mL×3). The combined organic phase was washed with brine (30 mL), dried over anhydrous Na₂SO₄, filtered and concentrated. The residue was purified by flash silica gel chromatography (eluent of 0-10% EtOAc in PE) to give tert-butyl (2-fluoro-3-methyl-4-((3-nitro-6-phenylpyridin-2-yl)amino)phenyl)carbamate (520 mg, 71.2% yield) as a yellow oil. ¹H NMR (400 MHz, DMSO-d₆) δ_{H}=9.95 (s, 1H), 8.94 (s, 1H), 8.61 (d, J=8.4 Hz, 1H), 7.97 (dd, J=1.6, 7.6 Hz, 2H), 7.58-7.44 (m, 6H), 2.16 (d, J=2.0 Hz, 3H), 1.48 (s, 9H).

Step 2. To a solution of tert-butyl (2-fluoro-3-methyl-4-((3-nitro-6-phenylpyridin-2-yl)amino)phenyl)carbamate (550 mg, 1.25 mmol) in DCM (8 mL), TFA (1.5 mL, 1.25 mmol) was added at 25° C. and the mixture was stirred for 12 hrs. The reaction mixture was concentrated directly to give 3-fluoro-2-methyl-N1-(3-nitro-6-phenylpyridin-2-yl)benzene-1,4-diamine (450 mg, 1.33 mmol) as a yellow solid, which was used in the next step directly. ¹H NMR (400 MHz, DMSO-d₆) δ_{H}=9.79 (s, 1H), 8.57 (d, J=8.8 Hz, 1H), 7.94 (dd, J=2.0, 8.0 Hz, 2H), 7.53-7.42 (m, 4H), 7.16 (d, J=8.4 Hz, 1H), 6.75 (t, J=9.2 Hz, 1H), 2.09 (d, J=2.0 Hz, 3H).

Step 3. To a solution of 3-fluoro-2-methyl-NI-(3-nitro-6-phenylpyridin-2-yl)benzene-1,4-diamine (225 mg, 0.665 mmol) in pyridine (3 mL), 6-(methoxycarbonyl)spiro[3.3]heptane-2-carboxylic acid (158 mg, 0.798 mmol) and EDCI (153 mg, 0.798 mmol) were added at 25° C. and the mixture was stirred at 40° C. for 16 hrs. Water (20 mL) was added to the reaction and the mixture was stirred for 2 hrs. The reaction mixture was then filtered. The filter cake was washed with EtOAc (5 mL), water (5 mL) and concentrated to give methyl 6-((2-fluoro-3-methyl-4-((3-nitro-6-phenylpyridin-2-yl)amino)phenyl)carbamoyl)spiro[3.3]heptane-2-carboxylate (190 mg, 55.1% yield) as a red solid, which was used in the next step directly LCMS Rₜ=1.199 and 1.222 min in 1.5 min chromatography, purity 88.1%, MS ESI calcd. for 518.2 [M+H]⁺ 519.2, found 519.2.

Step 4. To a solution of methyl 6-[[2-fluoro-3-methyl-4-[(3-nitro-6-phenyl-2-pyridyl)amino]phenyl]carbamoyl]spiro[3.3]heptane-2-carboxylate (190 mg, 0.366 mmol) in ethyl acetate (10 mL), wet Pd/C (10%, 0.15 g) was added under Ar atmosphere. The suspension was degassed and purged with H₂ (×3) and the reaction mixture was stirred under H₂ (15 psi) at 25° C. for 16 hrs. The reaction mixture was filtered and the filter cake was washed with EtOAc (30 mL). The filtrate was concentrated to give methyl 6-((4-((3-amino-6-phenylpyridin-2-yl)amino)-2-fluoro-3-methylphenyl)carbamoyl)spiro[3.3]heptane-2-carboxylate (190 mg, 0.389 mmol) as a purple oil. The crude product was used in the next step directly. LCMS R'₂ 0.876 and 0.911 min in 1.5 min chromatography, purity 98.8%, MS ESI calcd. for 488.22 [M+H]⁺ 489.22, found 489.3.

Step 5. A solution of 2-aminonicotinaldehyde (95.0 mg, 0.778 mmol) and methyl 6-((4-((3-amino-6-phenylpyridin-2-yl)amino)-2-fluoro-3-methylphenyl)carbamoyl)spiro[3.3]heptane-2-carboxylate (190 mg, 0.389 mmol) in acetic acid (3 mL) was stirred at 80° C. for 2 hrs under 02. The reaction mixture was concentrated directly to give methyl 6-((4-(2-(2-aminopyridin-3-yl)-5-phenyl-3H-imidazo[4,5-b]pyridin-3-yl)-2-fluoro-3-methylphenyl)carbamoyl)spiro[3.3]heptane-2-carboxylate (240 mg, 51.3% yield) as a yellow solid, which was used in the next step directly. LCMS Rₜ=0.938 min in 1.5 min chromatography, purity 49.1%, MS ESI calcd for 590.2 [M+H]⁺ 591.2. found 591.3.

Step 6. To a solution of methyl 6-((4-(2-(2-aminopyridin-3-yl)-5-phenyl-3H-imidazo[4,5-b]pyridin-3-yl)-2-fluoro-3-methylphenyl)carbamoyl)spiro[3.3]heptane-2-carboxylate (280 mg, 0.474 mmol) in THF (4 mL), water (2 mL) and LiOH·H₂O (39 mg, 0.948 mmol) were added at 25° C. and the mixture was stirred for 2 hrs. The reaction mixture was filtered and the filter cake was washed with DMF (7 mL). The filtrate was concentrated. The residue was purified by prep-HPLC (column: Welch Xtimate C18 40*200 mm 7 μm; mobile phase: water (NH₄HCO₃)-ACN: gradient: 10%-50% B over 25 min) to give 6-((4-(2-(2-aminopyridin-3-yl)-5-phenyl-3H-imidazo[4,5-b]pyridin-3-yl)-2-fluoro-3-methylphenyl)carbamoyl)spiro[3.3]heptane-2-carboxylic acid I-101 (65.0 mg, 23.3% yield) as a light yellow solid. ¹H NMR (400 MHz, DMSO-d₆) δ_{H}=9.72 (s, 1H), 8.29 (d, J=8.4 Hz, 1H), 8.06-7.94 (m, 5H), 7.50-7.43 (m, 2H), 7.40 (d, J=7.2 Hz, 1H), 7.30-7.19 (m, 3H), 7.11 (dd, J=1.6, 7.6 Hz, 1H), 6.42 (dd, J=4.8, 7.6 Hz, 1H), 3.29-3.23 (m, 1H), 2.93 (m, J=8.4 Hz, 1H), 2.20 (m, 4H), 2.19-2.07 (m, 4H), 1.89 (d, J=1.6 Hz, 3H).

### Example 93. Synthesis of (1r,4r)-4-((4-(2-(2-aminopyridin-3-yl)-5-phenyl-3H-imidazo[4,5-b]pyridin-3-yl)-3-methylphenyl)carbamoyl)cyclohexane-1-carboxylic acid (Compound I-102)

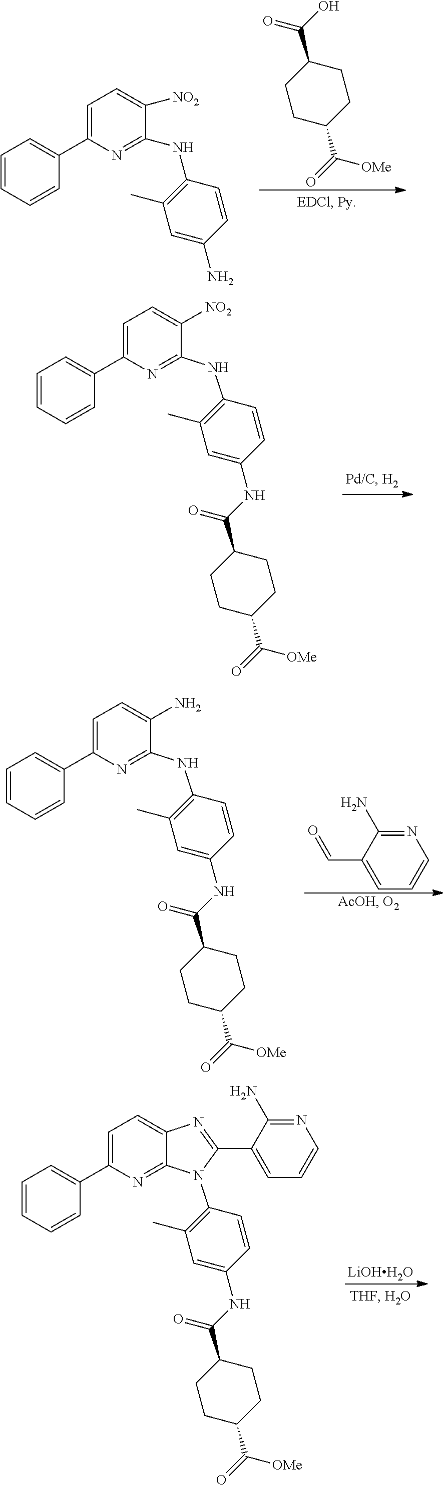

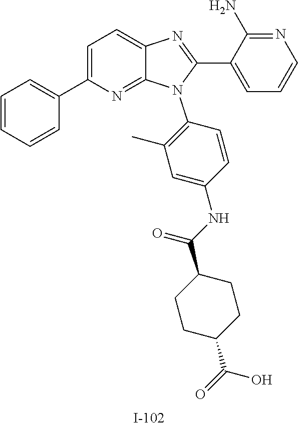 [SMILES: Cc1cc(NC(=O)[C@H]2CC[C@H](C(=O)O)CC2)ccc1-n1c(-c2cccnc2N)nc2ccc(-c3ccccc3)nc21]

Step 1. To a solution of 2-methyl-NP-(3-nitro-6-phenylpyridin-2-yl)benzene-1,4-diamine (300 mg, 0.936 mmol) and (1r,4r)-4-(methoxycarbonyl)cyclohexane-1-carboxylic acid (174 mg, 0.936 mmol) in Pyridine (8 mL), EDCI (269 mg, 1.40 mmol) was added and the mixture was stirred at 25° C. for 2 hrs. Water (5 mL) was added to the residue and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent of 10-20% EtOAc in PE) to give methyl (1r,4r)-4-((3-methyl-4-((3-nitro-6-phenylpyridin-2-yl)amino)phenyl)carbamoyl)cyclohexane-1-carboxylate (100 mg, 21.9% yield) as a red solid. ¹H NMR (DMSO-d₆, 400 MHz) δ_{H}=9.94-9.86 (m, 2H), 8.59 (d, J=8.8 Hz, 1H), 8.02-7.97 (m, 3H), 7.69 (d, J=8.8 Hz, 1H), 7.58 (d, J=2.4 Hz, 1H), 7.51-7.48 (m, 4H), 3.61 (s, 3H), 2.52-2.51 (m, 1H), 2.34-2.32 (m, 1H), 2.24 (s, 3H), 2.01-1.97 (m, 2H), 1.92-1.87 (m, 2H), 1.54-1.45 (m, 2H), 1.43-1.35 (m, 2H).

Step 2. To a solution of methyl (1r,4r)-4-((3-methyl-4-((3-nitro-6-phenylpyridin-2-yl)amino)phenyl)carbamoyl)cyclohexane-1-carboxylate (100 mg, 0.205 mmol) in THF (10 mL), Pd/C (50 mg, purity 10%) was added under N₂. The reaction mixture was degassed and purged with H₂ (15 psi) (×3) and the mixture was stirred at 25° C. for 12 hrs under H₂ (15 psi). The reaction mixture was filtered and the filtrate was concentrated directly to give methyl (1r,4r)-4-((4-((3-amino-6-phenylpyridin-2-yl)amino)-3-methylphenyl)carbamoyl)cyclohexane-1-carboxylate (100 mg, 0.194 mmol) as a light yellow oil, which was used in the next step directly. LCMS &=0.581 min in 1 min chromatography, purity 89.1%, MS ESI calcd. For 458.23, [M+H]⁺ 459.23, found 459.2.

Step 3. To a solution of methyl (1r,4r)-4-((4-((3-amino-6-phenylpyridin-2-yl)amino)-3-methylphenyl)carbamoyl)cyclohexane-1-carboxylate (100 mg, 0.218 mmol) in acetic acid (3 mL), 2-aminonicotinaldehyde (32 mg, 0.262 mmol) was added under 02. The mixture was stirred at 80° C. for 1 hr under O₂. The reaction mixture was concentrated directly to give methyl (1r,4r)-4-((4-(2-(2-aminopyridin-3-yl)-5-phenyl-3H-imidazo[4,5-b]pyridin-3-yl)-3-methylphenyl)carbamoyl)cyclohexane-1-carboxylate (100 mg, 73.6% yield) as a light yellow oil, which was used in the next step directly. LCMS &=0.498 min in 1 min chromatography, purity 94.0%, MS ESI calcd. for 560.25, [M+H]⁺ 561.25, found 561.3.

Step 4. To a solution of methyl (1r,4r)-4-((4-(2-(2-aminopyridin-3-yl)-5-phenyl-3H-imidazo[4,5-b]pyridin-3-yl)-3-methylphenyl)carbamoyl)cyclohexane-1-carboxylate (100 mg, 0.178 mmol) in THF (3 mL) and water (3 mL), LiOH·H₂O (15 mg, 0.357 mmol) was added at 25° C. and the mixture was stirred for 12 hrs. The mixture was filtered directly. The residue was purified by prep-HPLC (Column. Welch Xtimate C18 150*30 mm*5 um; mobile phase: [water (NH₄HCO₃)-ACN]; B %: 6%-46%; 25 min) to give (1r,4r)-4-((4-(2-(2-aminopyridin-3-yl)-5-phenyl-3H-imidazo[4,5-b]pyridin-3-yl)-3-methylphenyl)carbamoyl)cyclohexane-1-carboxylic acid 1-102 (46.0 mg, 46.9% yield) as a light yellow solid. ¹H NMR (DMSO-d₆, 400 MHz) δ_{H}=10.07 (s, 1H), 8.27 (d, J=8.4 Hz, 1H), 8.03-7.94 (m, 4H), 7.75 (d, J=2.0 Hz, 1H), 7.59 (dd, J=2.0, 8.8 Hz, 1H), 7.48-7.42 (m, 2H), 7.40-7.28 (m, 4H), 7.11 (dd, J=1.6, 7.6 Hz, 1H), 6.40 (dd, J=4.8, 7.6 Hz, 1H), 2.35-2.31 (m, 1H), 2.24-2.16 (m, 1H), 2.02-1.96 (m, 2H), 1.94-1.87 (m, 5H), 1.54-1.43 (m, 2H), 1.41-1.30 (m, 2H). HPLC Rt=2.67 min in 8 min chromatography, purity 99.6%. LCMS Rt=1.24 min in 2 min chromatography, purity 99.3%, MS ESI calcd. for 546.24, [M+H]⁺ 547.24, found 547.2.

### Example 94. Synthesis of (1r,4r)-4-((4-(2-(2-aminopyridin-3-yl)-5-phenyl-3H-imidazo[4,5-b]pyridin-3-yl)-2-fluoro-3-methylphenyl)carbamoyl)cyclohexane-1-carboxylic acid (Compound I-103)

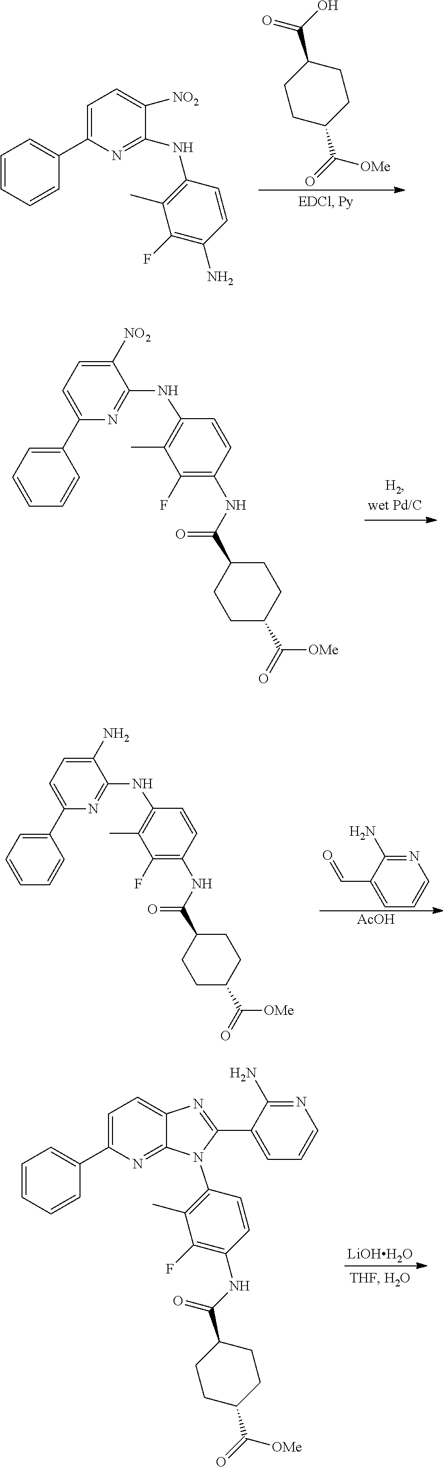

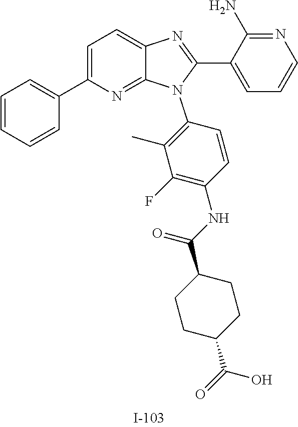 [SMILES: Cc1c(-n2c(-c3cccnc3N)nc3ccc(-c4ccccc4)nc32)ccc(NC(=O)[C@H]2CC[C@H](C(=O)O)CC2)c1F]

Step 1. To a solution of 3-fluoro-2-methyl-N1-(3-nitro-6-phenylpyridin-2-yl)benzene-1,4-diamine (225 mg, 0.665 mmol) in pyridine (3 mL), (1r,4r)-4-(methoxycarbonyl)cyclohexane-1-carboxylic acid (186 mg, 0.998 mmol) and EDCI (191 mg, 0.998 mmol) were added at 25° C. and the mixture was stirred at 60° C. for 16 hrs. Water (10 mL) was added and the mixture allowed to stir for 2 hrs. The reaction mixture was filtered and the filter cake was washed with water (5 mL) and concentrated to give methyl (1r,4r)-4-((2-fluoro-3-methyl-4-((3-nitro-6-phenylpyridin-2-yl)amino)phenyl)carbamoyl)cyclohexane-1-carboxylate (200 mg, 59.4% yield) as a red solid, which was used in the next step directly. LCMS Rₜ=1.203 min in 1.5 min chromatography, purity 96.18%, MS ESI calcd. for 506.20, [M+H]⁺ 507.20, found 507.2.

Step 2. To a solution of methyl (1r,4r)-4-((2-fluoro-3-methyl-4-((3-nitro-6-phenylpyridin-2-yl)amino)phenyl)carbamoyl)cyclohexane-1-carboxylate (200 mg, 0.395 mmol) in ethyl acetate (10 mL), Pd/C (10%, 0.15 g) was added under N₂ atmosphere. The suspension was degassed and purged with H₂ (×3) and the reaction mixture was stirred under H₂ (15 psi) at 25° C. for 16 hrs. The reaction mixture was filtered. The filter cake was washed with EtOAc (30 mL). The filtrate was concentrated to give trans-methyl 4-[[4-[(3-amino-6-phenyl-2-pyridyl)amino]-2-fluoro-3-methyl-phenyl]carbamoyl]cyclohexanecarboxylate (180 mg, 95.7% yield) as a purple oil, which was used in the next step directly LCMS Rₜ=0.858 min in 1.5 min chromatography, purity 41.11%, MS ESI calcd. for 476.22, [M+H]⁺ 477.22, found 477.2.

Step 3. A solution of 2-aminonicotinaldehyde (92.0 mg, 0.755 mmol) and methyl (1r,4r)-4-((4-((3-amino-6-phenylpyridin-2-yl)amino)-2-fluoro-3-methylphenyl)carbamoyl)cyclohexane-1-carboxylate (180 mg, 0.378 mmol) in acetic acid (3 mL) was stirred at 80° C. for 2 hrs under 02. The reaction mixture was concentrated directly to give methyl (1r,4r)-4-((4-(2-(2-aminopyridin-3-yl)-5-phenyl-3H-imidazo[4,5-b]pyridin-3-yl)-2-fluoro-3-methylphenyl)carbamoyl)cyclohexane-1-carboxylate (300 mg, 71.5% yield) as a yellow solid, which was used in the next step directly. LCMS Rₜ=0.920 min in 1.5 min chromatography, purity 52.1%, MS ESI calcd. for 578.24, [M+H]⁺ 579.24, found 579.3.

Step 4. To a solution of methyl (1r,4r)-4-((4-(2-(2-aminopyridin-3-yl)-5-phenyl-3H-imidazo[4,5-b]pyridin-3-yl)-2-fluoro-3-methylphenyl)carbamoyl)cyclohexane-1-carboxylate (300 mg, 0.518 mmol) in THF (4 mL), water (2 mL) and LiOH·H₂O (43 mg, 1.04 mmol) were added at 25° C. and the mixture was stirred for 2 hrs. The reaction mixture was filtered. The filter cake was washed with DMF (7 mL). The filtrate was concentrated. The residue was purified by prep-HPLC (column: Welch Xtimate C18 40*200 mm 7 μm; mobile phase: water (NH₄HCO₃)-ACN; gradient: 8%-48% B over 25 min) to give (1r,4r)-4-((4-(2-(2-aminopyridin-3-yl)-5-phenyl-3H-imidazo[4,5-b]pyridin-3-yl)-2-fluoro-3-methylphenyl)carbamoyl)cyclohexane-1-carboxylic acid I-103 (45.0 mg, 15.1% yield) as a light yellow solid. ¹H NMR (DMSO-d₆, 400 MHz) δ_{H}=12.08 (s, 1H), 9.82 (s, 1H), 8.30 (d, J=8.4 Hz, 1H), 8.04-7.92 (m, 5H), 7.51-7.36 (m, 3H), 7.30-7.19 (m, 3H), 7.12 (dd, J=1.6, 7.6 Hz, 1H), 6.43 (dd, J=4.8, 8.0 Hz, 1H), 2.56 (m, 1H), 2.28-2.16 (m, 1H), 2.01-1.89 (m, 7H), 1.48 (q, J=12.4 Hz, 2H), 1.41-1.31 (m, 2H). HPLC Rₜ=2.757 min in 8 min chromatography, purity 98.9%. LCMS Rₜ=2.068 min in 4 min chromatography, purity 99.0%, MS ESI calcd. for 564.23, [M+H]⁺ 565.23, found 565.3.

### Example 95. Synthesis of 2-[1-[[4-[2-(2-amino-3-pyridyl)-5-phenyl-imidazo[4,5-b]pyridin-3-yl]phenyl]methyl]pyrrolidin-3-yl]propanoic acid (Compound I-104)

### Synthesis of methyl 2-methyl-2-pyrrolidin-3-yl-propanoate and methyl 2-pyrrolidin-3-ylpropanoate

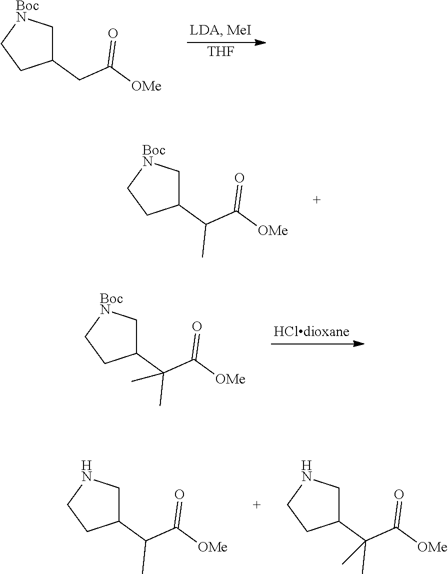 [SMILES: COC(=O)CC1CCN(C(=O)OC(C)(C)C)C1 ; COC(=O)C(C)C1CCN(C(=O)OC(C)(C)C)C1 ; COC(=O)C(C)(C)C1CCN(C(=O)OC(C)(C)C)C1 ; COC(=O)C(C)C1CCNC1 ; COC(=O)C(C)(C)C1CCNC1 ; "THF"]

Step 1. To a stirred solution of tert-butyl 3-(2-methoxy-2-oxo-ethyl)pyrrolidine-1-carboxylate (400 mg, 1.64 mmol) in THF (5 mL), LDA (2 M, 3 mL) was added slowly at −78° C. and the mixture was stirred at −78° C. under N₂ for 1 hr. Mel (0.51 mL, 8.22 mmol) was then added to the reaction mixture, and the resulting mixture was stirred at 20° C. under N₂ for 1 hr. The reaction mixture was quenched with water (10 mL) and extracted with EtOAc (15 mL×2). The combined organic layers were washed with brine (20 mL), dried over anhydrous sodium sulfate, filtered and the filtrate was concentrated to afford a residue. The crude product was purified by flash chromatography on silica gel (0-100% of EtOAc in PE) to afford a mixture of tert-butyl 3-(2-methoxy-1,1-dimethyl-2-oxo-ethyl)pyrrolidine-1-carboxylate and tert-butyl 3-(2-methoxy-1-methyl-2-oxo-ethyl)pyrrolidine-1-carboxylate (130 mg, 30.7% yield) as a yellow oil.

Step 2. To a mixture of tert-butyl 3-(2-methoxy-1,1-dimethyl-2-oxo-ethyl)pyrrolidine-1-carboxylate and tert-butyl 3-(2-methoxy-1-methyl-2-oxo-ethyl)pyrrolidine-1-carboxylate (65.0 mg, 0.253 mmol) in 1,4-dioxane (1 mL), HCl/dioxane (3 mL) was added and the mixture was stirred at 25° C. for 16 hrs. The mixture was concentrated to give a mixture of methyl 2-methyl-2-pyrrolidin-3-yl-propanoate and methyl 2-pyrrolidin-3-ylpropanoate (40 mg, crude) as a yellow solid.

### Synthesis of 2-[1-[[4-[2-(2-amino-3-pyridyl)-5-phenyl-imidazo[4,5-b]pyridin-3-yl]phenyl]methyl]pyrrolidin-3-yl]propanoic acid (Compound I-104)

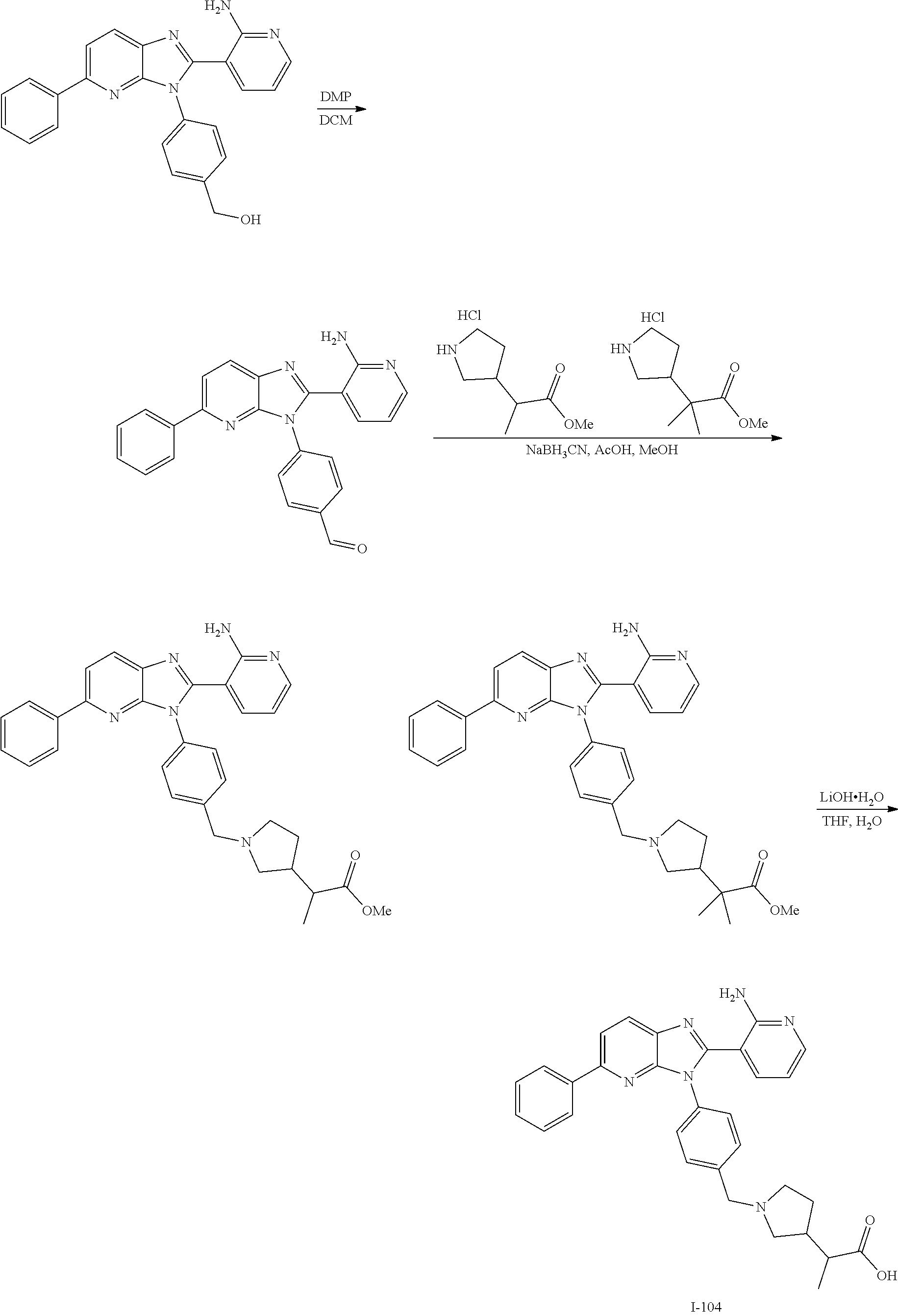

Step 1. To a solution of [4-[2-(2-amino-3-pyridyl)-5-phenyl-imidazo[4,5-b]pyridin-3-yl]phenyl]methanol (500 mg, 1.27 mmol) in THF (5 mL) and DCM (10 mL), DMP (1.05 mg, 2.54 mmol) was added at 0° C. and the mixture was stirred at 25° C. for 2 hrs. The mixture was quenched with 1:1 (1M Na₂S₂O₃:NaHCO₃), and further stirred for 30 min. Water (50 mL) was added and the mixture was extracted with DCM (50 mL×3). The organic phase was washed with brine (50 mL), dried over Na₂SO₄, filtered and concentrated under reduced pressure to give 4-[2-(2-amino-3-pyridyl)-5-phenyl-imidazo[4,5-b]pyridin-3-yl]benzaldehyde (500 mg, crude) as a brown solid, which was used in the next step without further purification. ¹H NMR (DMSO-d₆, 400 MHz) δ_{H}=10.09 (s, JH), 8.06 (br dd, J=4.8, 12.4 Hz, 6H), 7.75 (d, J=8.4 Hz, 2H), 7.47 (t, J=7.2 Hz, 4H), 6.88-6.81 (m, 2H), 6.46 (dd, J=4.8, 7.6 Hz, 1H).

Step 2. To a solution of 4-[2-(2-amino-3-pyridyl)-5-phenyl-imidazo[4,5-b]pyridin-3-yl]benzaldehyde (500 mg, 1.28 mmol) in methanol (10 mL), methyl 2-methyl-2-pyrrolidin-3-yl-propanoate and methyl 2-pyrrolidin-3-ylpropanoate (500 mg, mixture) and AcOH (38 mg, 0.639 mmol) were added and the mixture was stirred at 25° C. for 20 mins NaBH₃CN (812 mg, 3.83 mmol) was then added to the mixture and the mixture was stirred at 70° C. for 2 hrs. The reaction mixture was adjusted to pH ~7 by sat aq NaHCO₃ and the resulting mixture was extracted with EtOAc (20 mL×2). The combined organic phase was washed with brine (5 mL), dried over anhydrous Na₂SO₄, filtered and concentrated. The residue was purified by flash silica gel chromatography (eluent of 0-7% MeOH in DCM) to give methyl 2-[l-[[4-[2-(2-amino-3-pyridyl)-5-phenyl-imidazo[4,5-b]pyridin-3-yl]phenyl]methyl]pyrrolidin-3-yl]propanoate (40.0 mg, 26.7% yield) as a white solid. LCMS Rₜ=0.382 min in 1.0 min chromatography, purity 98.79%, MS ESI calcd. for 532.26 [M+H]⁺ 533.26, found 533.3. Methyl 2-[l-[[4-[2-(2-amino-3-pyridyl)-5-phenyl-imidazo[4,5-b]pyridin-3-yl]phenyl]methyl]pyrrolidin-3-yl]-2-methyl-propanoate (10 mg, 0.0183 mmol, 6.50% yield) was obtained as a white solid. LCMS Rₜ=0.401 min in 1.0 min chromatography, purity 97.9%, MS ESI calcd. for 546.27 [M+H]+ 547.27, found 547.3.

Step 3. To a solution of methyl 2-[1-[[4-[2-(2-amino-3-pyridyl)-5-phenyl-imidazo[4,5-b]pyridin-3-yl]phenyl]methyl]pyrrolidin-3-yl]propanoate (40 mg, 0.0751 mmol) in THF (I mL), LiOH·H₂O (9.5 mg, 0.225 mmol) and water (0.5 mL) were added and the mixture was stirred at 25° C. for 2 hrs. The reaction mixture was filtered, and the filtrate was concentrated to dryness. The residue was purified by prep-HPLC (Column: Welch Xtimate C18 40*200 mm 7 μm; mobile phase: [water (NH₄HCO₃)-ACN];B %: 8%-48%,25 min) to give 2-[1-[[4-[2-(2-amino-3-pyridyl)-5-phenyl-imidazo[4,5-b]pyridin-3-yl]phenyl]methyl]pyrrolidin-3-yl]propanoic acid I-104 (38.5 mg, 97.90% yield) as a white solid. ¹H NMR (DMSO-d₆, 400 MHz) δ_{H}=8.26 (d, J=8.4 Hz, 1H), 8.06-7.95 (m, 4H), 7.52-7.34 (m, 7H), 7.15 (dt, J=1.6, 8.4 Hz, 1H), 7.03 (br d, J=4.0 Hz, 2H), 6.38 (ddd, J=2.4, 4.8, 7.6 Hz, 1H), 3.72-3.59 (m, 2H), 2.71-2.66 (m, 1H), 2.61-2.55 (m, 1H), 2.48-2.41 (m, 1H), 2.30-2.17 (m, 3H), 1.96-1.85 (m, 1H), 1.52-1.42 (m, 1H), 1.06 (dd, J=6.0, 13.2 Hz, 3H). HPLC Rₜ=2.090 min in 8 min chromatography, purity 99.1%. LCMS Rₜ=0.951 min in 2.0 min chromatography, purity 98.1%, MS ESI calcd for 518.24 [M+H]⁺ 519.24, found 519.1.

### Example 96. Synthesis of 2-[1-[[4-[2-(2-amino-3-pyridyl)-5-phenyl-imidazo[4,5-b]pyridin-3-yl]phenyl]methyl]pyrrolidin-3-yl]-2-methyl-propanoic acid (Compound I-105)

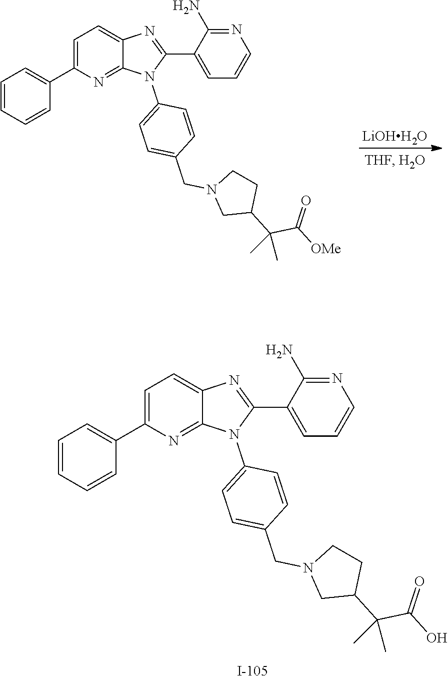 [SMILES: COC(=O)C(C)(C)C1CCN(Cc2ccc(-n3c(-c4cccnc4N)nc4ccc(-c5ccccc5)nc43)cc2)C1 ; "LiOH·H2O" ; CC(C)(C(=O)O)C1CCN(Cc2ccc(-n3c(-c4cccnc4N)nc4ccc(-c5ccccc5)nc43)cc2)C1]

Step 1. To a solution of methyl 2-[1-[[4-[2-(2-amino-3-pyridyl)-5-phenyl-imidazo[4,5-b]pyridin-3-yl]phenyl]methyl]pyrrolidin-3-yl]-2-methyl-propanoate (10.0 mg, 0.0183 mmol) in THF (1 mL), LiOH·H₂O (2.3 mg, 0.0549 mmol) and water (0.5 mL) were added and the mixture was stirred at 25° C. for 2 hrs. The reaction mixture was filtered, and the filtrate was concentrated to dryness. The residue was purified by prep-HPLC (Column: Welch Xtimate C18 40*200 mm 7 um; mobile phase: [water (NH₄HCO₃)-ACN]; B %: 10%-50%/o, 25 min) to give 2-[1-[[4-[2-(2-amino-3-pyridyl)-5-phenyl-imidazo[4,5-b]pyridin-3-yl]phenyl]methyl]pyrrolidin-3-yl]-2-methyl-propanoic acid I-105 (5.1 mg, 51.9% yield) as a yellow solid. ¹H NMR (400 MHz, DMSO-d₆) δ_{H}=8.26 (d, J=8.4 Hz, 1H), 8.07-7.93 (m, 4H), 7.51-7.35 (m, 7H), 7.14 (dd, J=1.6, 7.6 Hz, 1H), 7.04 (s, 2H), 6.37 (dd, J=4.8, 7.6 Hz, 1H), 3.71 (d, J=13.2 Hz, 1H), 3.57 (d, J=13.2 Hz, 1H), 2.53 (s, 2H), 2.47-2.38 (m, 2H), 2.34-2.30 (m, 1H), 1.85-1.70 (m, 1H), 1.64-1.52 (m, 1H), 1.05 (s, 6H). HPLC Rₜ=2.309 min in 8 min chromatography, purity 99.1%. LCMS Rₜ=0.994 min in 2.0 min chromatography, purity 99.4%, MS ESI calcd for 532.26 [M+H]⁺ 533.26, found 533.2.

### Example 97. Synthesis of 1-[[4-[2-(2-amino-3-pyridyl)-5-phenyl-imidazo[4,5-b]pyridin-3-yl]-2-fluoro-phenyl]carbamoyl]piperidine-4-carboxylic acid (Compound I-106)

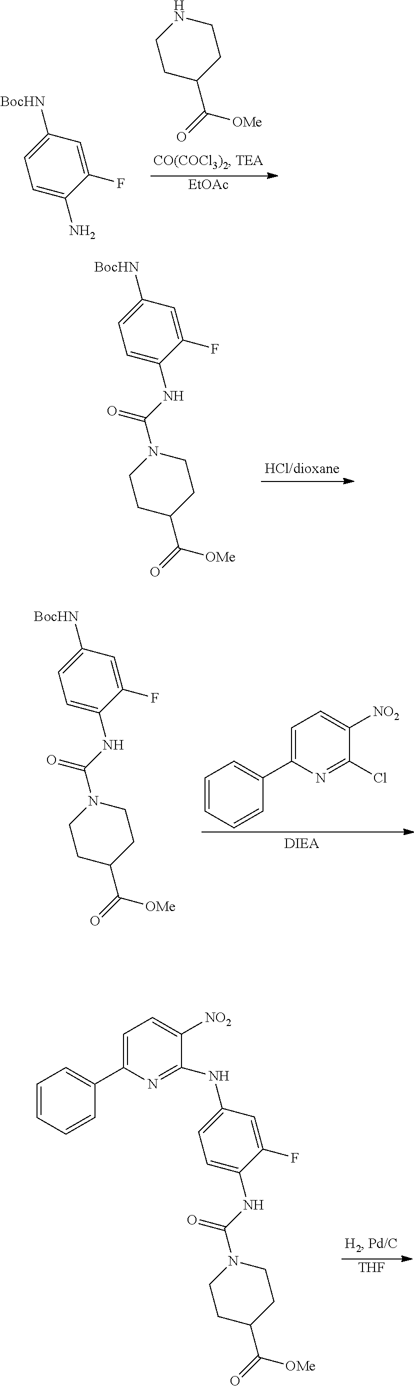

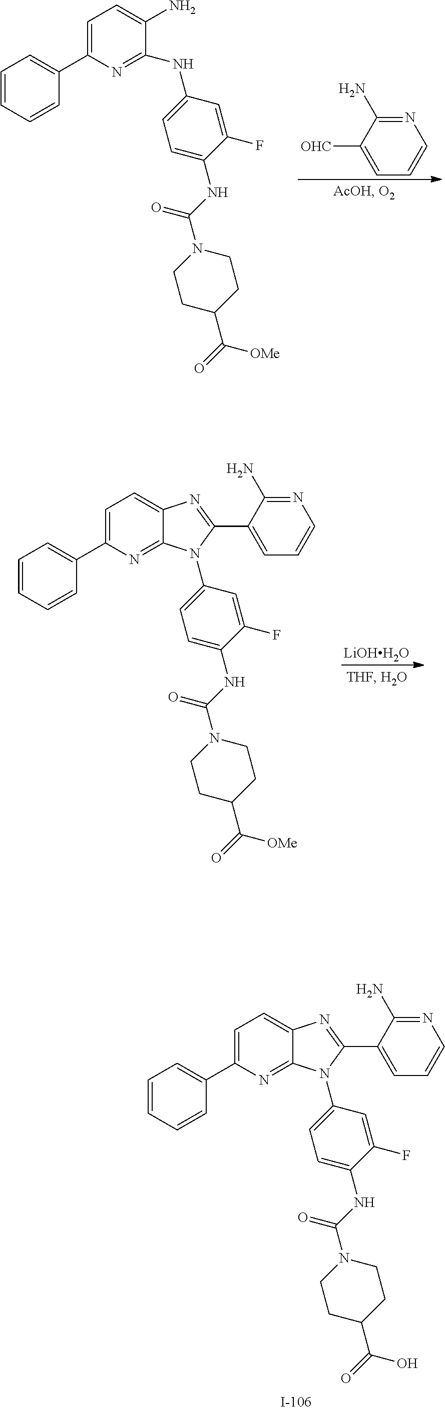 [SMILES: COC(=O)C1CCN(C(=O)Nc2ccc(Nc3nc(-c4ccccc4)ccc3N)cc2F)CC1 ; Nc1ncccc1C=O ; COC(=O)C1CCN(C(=O)Nc2ccc(-n3c(-c4cccnc4N)nc4ccc(-c5ccccc5)nc43)cc2F)CC1 ; "LiOH·H2O" ; Nc1ncccc1-c1nc2ccc(-c3ccccc3)nc2n1-c1ccc(NC(=O)N2CCC(C(=O)O)CC2)c(F)c1]

Step 1. To a solution of CO(OCCl₃)₂ (1.97 g, 6.63 mmol) in ethyl acetate (20 mL), a solution of tert-butyl (4-amino-3-fluorophenyl)carbamate (1.50 g, 6.63 mmol) and TEA (671 mg, 6.63 mmol) in ethyl acetate (20 mL) were added at 0° C. under N₂ atmosphere. The mixture was stirred at 25° C. for 1 hr, and then subsequently stirred at 90° C. for 5 hrs. A solution of methyl piperidine-4-carboxylate (1.90 g, 13.3 mmol) and TEA (671 mg, 6.63 mmol) in ethyl acetate (20 mL) was added into the mixture and the mixture was stirred at 90° C. for 12 hrs under N₂ atmosphere. Water (15 mL) was added and the resulting mixture was extracted with EtOAc (25 mL×3). The combined organic phase was washed with brine (15 mL), dried over anhydrous Na₂SO₄, filtered and concentrated. The residue was purified by Combi-Flash column (EtOAc in PE=0%-40%) to afford methyl 1-((4-((tert-butoxycarbonyl)amino)-2-fluorophenyl)carbamoyl)piperidine-4-carboxylate (1.30 g, 49.6% yield) as a yellow solid. ¹H NMR (400 MHz, DMSO-d₆) δ_{H}=9.45 (s, 1H), 8.12 (s, 1H), 7.34 (d, J=12.8 Hz, 1H), 7.23-7.15 (m, 1H), 7.10 (dd, J=1.6, 8.8 Hz, 1H), 3.96 (d, J=13.2 Hz, 2H), 3.62 (s, 3H), 2.97-2.83 (m, 2H), 2.63-2.54 (m, 1H), 1.82 (dd, J=2.8, 13.2 Hz, 2H), 1.47 (s, 1H).

Step 2. A solution of methyl 1-((4-((tert-butoxycarbonyl)amino)-2-fluorophenyl)carbamoyl)piperidine-4-carboxylate (1.27 g, 3.21 mmol) and HCl/dioxane (13 mL, 2 M) in DCM (0.50 mL) was stirred at 25° C. for 12 hrs. The mixture was concentrated directly to afford methyl 1-((4-amino-2-fluorophenyl)carbamoyl)piperidine-4-carboxylate (1.01 g, crude) as a yellow solid. ¹H NMR (400 MHz, DMSO-d₆) δ_{H}=8.37 (s, 1H), 7.40 (t, J=8.8 Hz, 1H), 7.10 (dd, J=2.0, 11.2 Hz, 1H), 7.01 (d, J=8.4 Hz, 1H), 3.98 (d, J1=13.2 Hz, 2H), 3.62 (s, 3H), 2.96-2.86 (m, 2H), 2.64-2.54 (m, 1H), 1.83 (dd, J=2.8, 13.2 Hz, 2H), 1.56-1.40 (m, 2H).

Step 3. To a stirred solution of 2-chloro-3-nitro-6-phenyl-pyridine (715 mg, 3.05 mmol) and methyl 1-[(4-amino-2-fluoro-phenyl)carbamoyl]piperidine-4-carboxylate (1.00 g, 3.39 mmol) in DMSO (15 mL), DIEA (1.9 mL, 10.2 mmol) was added and the reaction mixture was stirred at 80° C. under N₂ for 15 hrs. The reaction mixture was diluted with EtOAc (100 mL), washed with water (100 mL) and brine (100 mL×2), dried over anhydrous sodium sulfate, filtered and the filtrate was concentrated to afford a residue. The crude product was purified by flash chromatography on silica gel (70-90% of EtOAc in PE) to afford methyl 1-[[2-fluoro-4-[(3-nitro-6-phenyl-2-pyridyl)amino]phenyl]carbamoyl]piperidine-4-carboxylate (220 mg, 13.2% yield) as a yellow solid. ¹H NMR (400 MHz, DMSO-d₆) δ_{H}=10.11 (s, 1H), 8.62 (d, J=8.4 Hz, 1H), 8.30 (s, 1H), 8.11 (dd, J=2.8, 6.8 Hz, 2H), 7.75 (dd, J=2.4, 12.8 Hz, 1H), 7.62 (d, J=8.4 Hz, 1H), 7.58-7.50 (m, 3H), 7.48-7.35 (m, 2H), 4.06-3.89 (m, 2H), 3.63 (s, 3H), 3.00-2.88 (m, 2H), 2.68-2.54 (m, 1H), 1.90-1.79 (m, 2H), 1.59-1.45 (m, 2H).

Step 4. To a stirred solution of methyl 1-[[2-fluoro-4-[(3-nitro-6-phenyl-2-pyridyl)amino]phenyl]carbamoyl]piperidine-4-carboxylate (220 mg, 0.45 mmol) in methanol (1 mL) and THF (5 mL), Pd/C (50 mg, wet by water 55%, 10%/o Pd) was added under argon. The reaction was degassed and purged with Ar (×3), then degassed and purged with H₂ (×3), and stirred under H₂ (30 psi) for 16 hrs at 30° C. The reaction mixture was filtered, and the cake was washed with THF (20 mL×3). The combined filtrate was concentrated in vacuum to give methyl 1-[[4-[(3-amino-6-phenyl-2-pyridyl)amino]-2-fluoro-phenyl]carbamoyl]piperidine-4-carboxylate (200 mg, 96.8% yield) as a yellow solid. LCMS Rₜ=0.863 min in 1.5 min chromatography, purity 77.6%, MS ESI calcd. for 463.20 [M+H]⁺ 464.20, found 464.3.

Step 5. A mixture of 2-aminopyridine-3-carbaldehyde (79.0 mg, 0.65 mmol) and methyl 1-[[4-[(3-amino-6-phenyl-2-pyridyl)amino]-2-fluoro-phenyl]carbamoyl]piperidine-4-carboxylate (200 mg, 0.43 mmol) in acetic acid (5 mL) was stirred at 80° C. under 02 for 0.5 hr. The reaction mixture was diluted with EtOAc (20 mL), washed with water (50 mL) and brine (50 mL×2), dried over anhydrous sodium sulfate, filtered and the filtrate was concentrated to afford a residue. The crude product was purified by flash chromatography on silica gel (95% of EtOAc in PE) to afford methyl 1-[[4-[2-(2-amino-3-pyridyl)-5-phenyl-imidazo[4,5-b]pyridin-3-yl]-2-fluoro-phenyl]carbamoyl]piperidine-4-carboxylate (100 mg, 40.9% yield) as a yellow solid. LCMS Rₜ=0.888 min in 1.5 min chromatography, purity 100%, MS ESI calcd. for 565.22 [M+Na]⁺ 566.22, found 566.3.

Step 6. To a stirred solution of methyl 1-[[4-[2-(2-amino-3-pyridyl)-5-phenyl-imidazo[4,5-b]pyridin-3-yl]-2-fluoro-phenyl]carbamoyl]piperidine-4-carboxylate (100 mg, 0.180 mmol) in THF (1 mL) and water (1 mL), LiOH·H₂O (21 mg, 0.53 mmol) was added and the reaction mixture was stirred at 25° C. under N₂ for 3 hrs. The reaction mixture was concentrated and adjusted to pH ~5 with IN HCl. The mixture was purified by prep-HPLC (column: Welch Xtimate C18 150*30 mm*5 um, method: water (FA)-ACN, begin B: 20, end B: 50) to afford 1-[[4-[2-(2-amino-3-pyridyl)-5-phenyl-imidazo[4,5-b]pyridin-3-yl]-2-fluoro-phenyl]carbamoyl]piperidine-4-carboxylic acid 1-106 (45.0 mg, 45.5% yield) as a white solid. ¹H NMR (400 MHz, DMSO-d₆) δ_{H}=12.36 (s, 1H), 8.48 (s, 1H), 8.27 (d, J=8.4 Hz, 1H), 8.09-7.94 (m, 4H), 7.60 (t, J=8.8 Hz, 1H), 7.53-7.35 (m, 4H), 7.31 (dd, J=2.0, 7.6 Hz, 1H), 7.22 (dd, J=2.0, 8.4 Hz, 1H), 6.91 (s, 2H), 6.47 (dd, J=4.8, 7.6 Hz, 1H), 4.01 (br d, J=13.6 Hz, 2H), 2.96 (br t, J=11.2 Hz, 2H), 2.46 (s, 1H), 1.95-1.78 (m, 2H), 1.61-1.39 (m, 2H). HPLC Rₜ=4.118 min in 8 min chromatography, purity 98.7%. LCMS Rₜ=4.109 min in 8 min chromatography, purity 98.9%, MS ESI calcd. for 551.21 [M+H]⁺ 552.21, found 552.2.

### Example 98. Synthesis of 2-(5-(2-(2-aminopyridin-3-yl)-5-phenyl-3H-imidazo[4,5-b]pyridin-3-yl)pyridin-2-yl)-2-azaspiro[3.3]heptane-6-carboxylic acid (Compound I-107)

### Synthesis of methyl 2-azaspiro[3.3]heptane-6-carboxylate

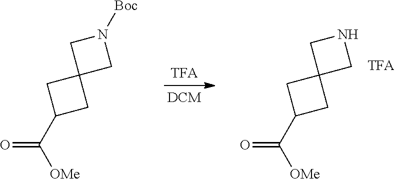 [SMILES: COC(=O)C1CC2(C1)CN(C(=O)OC(C)(C)C)C2 ; COC(=O)C1CC2(CNC2)C1]

Step 1. To a solution of 02-tert-butyl 06-methyl 2-azaspiro[3.3]heptane-2,6-dicarboxylate (300 mg, 1.18 mmol) in DCM (3.00 mL), TFA (3.00 mL) was added. After stirring at 25° C. for 3 hrs, the reaction mixture was concentrated in vacuum to give methyl 2-azaspiro[3.3]heptane-6-carboxylate TFA (900 mg, crude) as a yellow oil, which was used directly in the next step without further purification. ¹H NMR (400 MHz, DMSO-d₆) δ=8.65-8.54 (m, 2H), 3.97 (t, J=6.4 Hz, 2H), 3.89 (t, J=6.4 Hz, 2H), 3.59 (s, 3H), 3.05-2.95 (m, 1H), 2.48-2.41 (m, 2H), 2.37-2.29 (m, 2H).

### Synthesis of 2-(5-(2-(2-aminopyridin-3-yl)-5-phenyl-3H-imidazo[4,5-b]pyridin-3-yl)pyridin-2-yl)-2-azaspiro[3.3]heptane-6-carboxylic acid (Compound I-107)

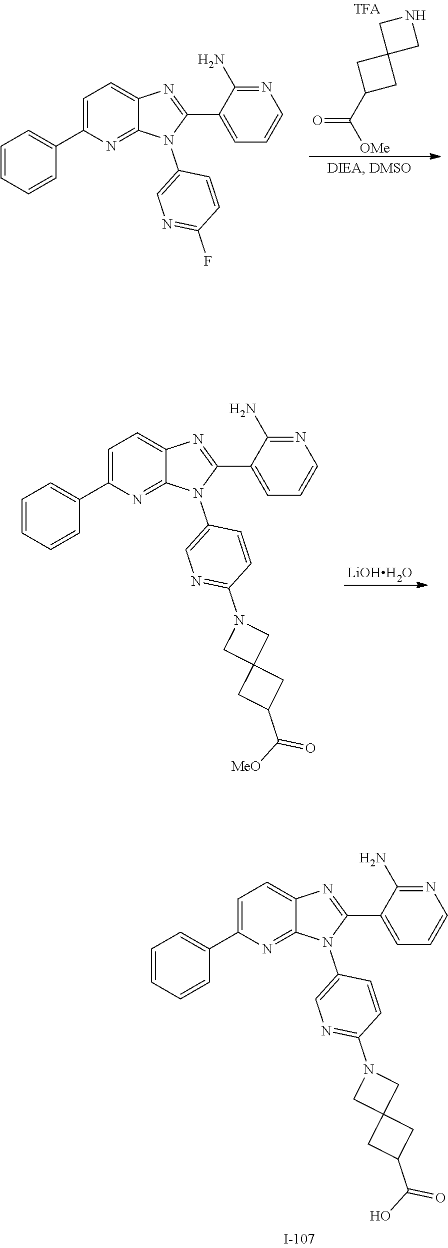

Step 1. To a solution of methyl 2-azaspiro[3.3]heptane-6-carboxylate TFA (100 mg, 0.395 mmol) in DMSO (3.00 mL), 3-[3-(6-fluoro-3-pyridyl)-5-phenyl-imidazo[4,5-b]pyridin-2-yl]pyridin-2-amine (227 mg, 0.592 mmol) and DIEA (0.210 mL, 1.18 mmol) were added and the reaction was then stirred at 100° C. for 16 hrs. Water (40 mL) was added to the reaction mixture and the whole extracted with EtOAc (30 mL×3). The organic layer was dried over Na₂SO₄, filtered, and concentrated in vacuum. The crude was purified by flash column (0-80% of EtOAc in PE) to give methyl 2-[5-[2-(2-amino-3-pyridyl)-5-phenyl-imidazo[4,5-b]pyridin-3-yl]-2-pyridyl]-2-azaspiro[3.3]heptane-6-carboxylate (100 mg, 48.9% yield) as a pink solid. ¹H NMR (400 MHz, DMSO-d₆) δ=8.24 (d, J=8.0 Hz, 1H), 8.14 (d, J=2.4 Hz, 1H), 8.07-7.94 (m, 4H), 7.61 (dd, J=8.8, 2.4 Hz, 1H), 7.50-7.32 (m, 4H), 6.86 (s, 2H), 6.52-6.43 (m, 2H), 4.06 (s, 2H), 3.96 (s, 2H), 3.62 (s, 3H), 3.10 (t, J=8.8 Hz, 1H), 2.46-2.31 (m, 4H).

Step 2. To a solution of methyl 2-[5-[2-(2-amino-3-pyridyl)-5-phenyl-imidazo[4,5-b]pyridin-3-yl]-2-pyridyl]-2-azaspiro[3.3]heptane-6-carboxylate (100 mg, 0.193 mmol) in THF (1.00 mL) and water (1.00 mL), LiOH H₂O (16.0 mg, 0.386 mmol) was added and the reaction mixture was stirred at 25° C. for 4 hrs. The reaction mixture was taken directly for purification by prep-HPLC (column: Welch Xtimate C18 150*30 mm*5 μm; mobile phase: water (FA)-ACN; B %: 12%-52%, 30 min) to give 2-[5-[2-(2-amino-3-pyridyl)-5-phenyl-imidazo[4,5-b]pyridin-3-yl]-2-pyridyl]-2-azaspiro[3.3]heptane-6-carboxylic acid 1-107 (57.0 mg, 58.4% yield) as a yellow solid. ¹H NMR (400 MHz, DMSO-d₆) δ=8.24 (d, J=8.4 Hz, 1H) 6.14 (d, J=2.4 Hz, 1H), 8.05-7.93 (m, 4H), 7.61 (dd, J=8.4, 2.4 Hz, 1H), 7.51-7.32 (m, 4H), 6.86 (s, 2H), 6.52-6.43 (m, 2H), 4.05 (s, 2H), 3.95 (s, 2H), 3.05-2.93 (m, 1H), 2.45-2.31 (m, 4H). LCMS Rₜ=2.883 min in 4 min chromatography, purity 100%, MS ESI calcd. For 503.21 [M+H]⁺ 504.21, found 504.1 HPLC Rₜ=3.622 min in 8 min chromatography, purity 100%.

### Example 99. Synthesis of 4-((5-(2-(2-aminopyridin-3-yl)-5-phenyl-3H-imidazo[4,5-b]pyridin-3-yl)-6-methylpyridin-2-yl)carbamoyl)cyclohexane-1-carboxylic acid (Compound I-108)

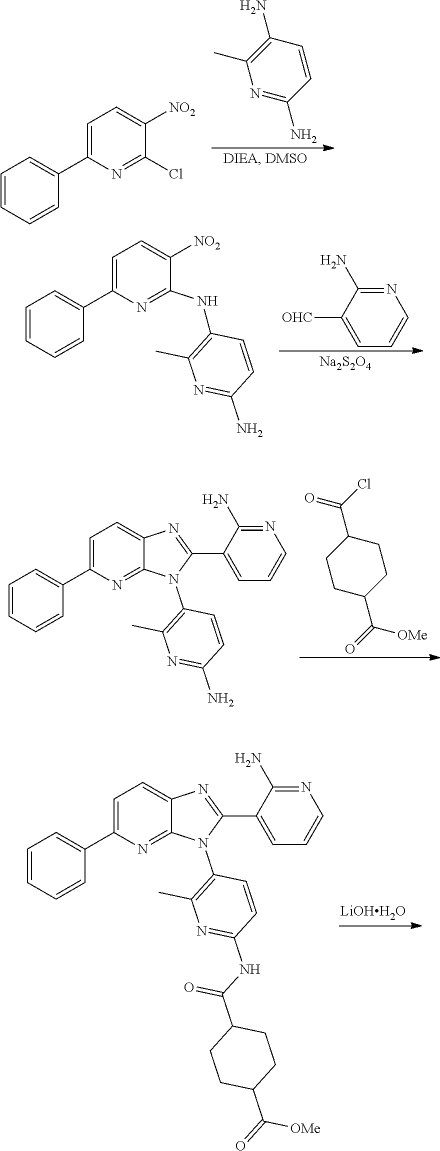

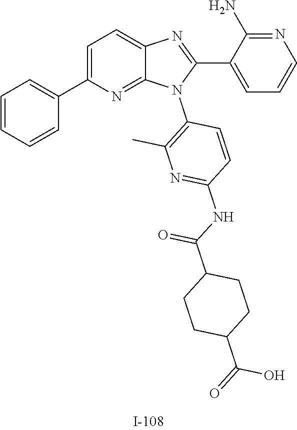 [SMILES: Cc1nc(NC(=O)C2CCC(C(=O)O)CC2)ccc1-n1c(-c2cccnc2N)nc2ccc(-c3ccccc3)nc21]

Step 1. To a stirred solution of 2-chloro-3-nitro-6-phenyl-pyridine (300 mg, 1.28 mmol) and 6-methylpyridine-2,5-diamine (200 mg, 1.62 mmol) in DMSO (10 mL), DIEA (1.2 mL, 6.39 mmol) was added and the reaction mixture was stirred at 100° C., under N₂, for 16 hrs. The reaction mixture was diluted with EtOAc (15 mL), washed with water (15 mL) and brine (20 mL), dried over anhydrous sodium sulfate, filtered and the filtrate was concentrated and purified by flash chromatography on silica gel (60% of EtOAc in PE) to afford 6-methyl-N5-(3-nitro-6-phenyl-2-pyridyl)pyridine-2,5-diamine (280 mg, 68.2% yield) as a yellow solid. ¹H NMR (400 MHz, DMSO-d₆) δ=9.71 (s, 1H), 8.56 (d, J=8.8 Hz, 1H), 8.02-7.85 (m, 2H), 7.56-7.39 (m, 5H), 6.37 (d, J=8.8 Hz, 1H), 5.95-5.79 (m, 2H), 3.30 (s, 3H).

Step 2. To a stirred solution of 2-aminopyridine-3-carbaldehyde (426 mg, 3.49 mmol) and 6-methyl-N5-(3-nitro-6-phenyl-2-pyridyl)pyridine-2,5-diamine (800 mg, 2.49 mmol) in DMSO (10 mL) and methanol (1 mL), Na₂S₂O₄ (1.73 g, 9.96 mmol) was added and the reaction mixture was stirred at 100° C., under N₂, for 16 hrs. The reaction mixture was diluted with EtOAc (20 mL), washed with water (20 mL) and brine (20 mL), dried over anhydrous sodium sulfate, filtered and the filtrate was concentrated and purified by flash chromatography on silica gel (95% of EtOAc in PE) to afford 5-[2-(2-amino-3-pyridyl)-5-phenyl-imidazo[4,5-b]pyridin-3-yl]-6-methyl-pyridin-2-amine (200 mg, 20.4% yield) as a yellow solid. ¹H NMR (400 MHz, DMSO-d₆) δ=8.26 (d, J=8.4 Hz, 1H), 8.13-7.87 (m, 4H), 7.51-7.44 (m, 2H), 7.43-7.35 (m, 2H), 7.32-7.17 (m, 3H), 6.54-6.37 (m, 2H), 6.30 (s, 2H), 1.87 (s, 3H).

Step 3. To a stirred solution of 5-[2-(2-amino-3-pyridyl)-5-phenyl-imidazo[4,5-b]pyridin-3-yl]-6-methyl-pyridin-2-amine (150 mg, 0.380 mmol) in DCM (2 mL), methyl 4-chlorocarbonylcyclohexanecarboxylate (195 mg, 0.95 mmol) was added at 0° C. and the reaction mixture was stirred at 25° C. under N₂ for 2 hrs. The reaction mixture was concentrated to remove DCM and purified by flash chromatography on silica gel (95% of EtOAc in PE) to afford methyl 4-[[5-[2-(2-amino-3-pyridyl)-5-phenyl-imidazo[4,5-b]pyridin-3-yl]-6-methyl-2-pyridyl]carbamoyl]cyclohexanecarboxylate (50 mg, 23.4% yield) ¹H NMR (400 MHz, DMSO-d₆) δ=10.66 (s, 1H), 8.30 (d, J=8.4 Hz, 1H), 8.10 (d, J=8.4 Hz, 1H), 8.05-7.95 (m, 4H), 7.86 (d, J=8.8 Hz, 1H), 7.54-7.34 (m, 3H), 7.25-7.10 (m, 3H), 6.46 (dd, J=4.8, 8.0 Hz, 1H), 3.64-3.58 (m, 3H), 2.67-2.58 (m, 2H), 2.06 (s, 3H), 2.04-2.00 (m, 1H), 1.75-1.23 (m, 7H).

Step 4. To a stirred solution of methyl 4-[[5-[2-(2-amino-3-pyridyl)-5-phenyl-imidazo[4,5-b]pyridin-3-yl]-6-methyl-2-pyridyl]carbamoyl]cyclohexanecarboxylate (50.0 mg, 0.09 mmol) in THF (1 mL) and water (1 mL), LiOH·H₂O (7.1 mg, 0.18 mmol) was added and the reaction mixture was stirred at 25° C. under N₂ for 5 hrs. The reaction mixture was adjusted to pH ~5 with 1N HCl and concentrated to afford a residue. The crude product was purified by prep-HPLC (column: Welch Xtimate C18 150×30 mm×5 μm, method: water (FA)-ACN, begin B: 30, end B 61) to afford 4-[[5-[2-(2-amino-3-pyridyl)-5-phenyl-imidazo[4,5-b]pyridin-3-yl]-6-methyl-2-pyridyl]carbamoyl]cyclohexanecarboxylic acid 1-108 (14.6 mg, 29.9% yield) as a white solid. ¹H NMR (400 MHz, DMSO-d₆) δ=12.14 (s, 1H), 10.88-10.45 (m, 1H), 8.31 (d, J=8.4 Hz, 1H), 8.11 (d, J=8.4 Hz, 1H), 8.06-7.94 (m, 4H), 7.87 (d, J=8.4 Hz, 1H), 7.55-7.05 (m, 6H), 6.51 (dd, J=5.2, 8.0 Hz, 1H), 2.68-2.57 (m, 1H), 2.26-1.82 (m, 6H), 1.72-1.19 (m, 6H). HPLC Rₜ=3.122 min in 8 min chromatography, purity 100%. LCMS Rₜ=2.530 min in 4 min chromatography, purity 100%, MS ESI calcd. for 547.23 [M+H]⁺ 548.23, found 548.1.

### Example 100. Synthesis of 4-((5-(2-(2-aminopyridin-3-yl)-5-phenyl-3H-imidazo[4,5-b]pyridin-3-yl)-6-methylpyridin-2-yl)amino)cyclohexane-1-carboxylic acid (Compound I-109)

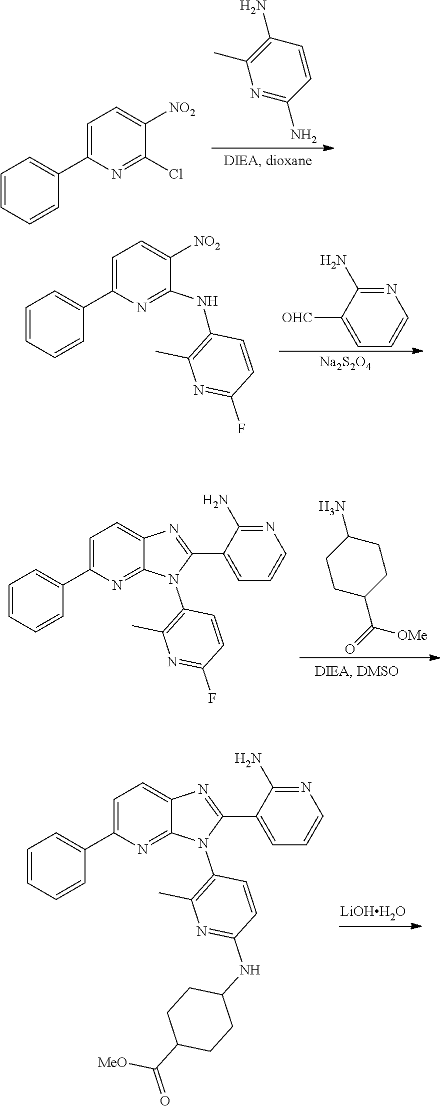

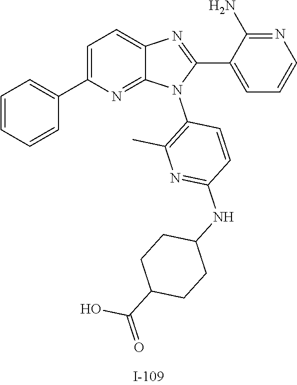 [SMILES: Cc1nc(NC2CCC(C(=O)O)CC2)ccc1-n1c(-c2cccnc2N)nc2ccc(-c3ccccc3)nc21]

Step 1. To a solution of 2-chloro-3-nitro-6-phenylpyridine (1.00 g, 4.26 mmol) in 1,4-dioxane (15 mL), 6-fluoro-2-methylpyridin-3-amine (1.075 g, 8.52 mmol) and DIEA were added and the reaction stirred at 100° C. for 16 hrs. Water (40 mL) was added to the mixture and the whole extracted with EtOAc (20 mL×3). The organic layer was dried over Na₂SO₄ and concentrated in vacuum to provide the crude material. The crude was purified by flash column (0-40% of EtOAc in PE) to give N-(6-fluoro-2-methylpyridin-3-yl)-3-nitro-6-phenylpyridin-2-amine (500 mg, 36.2% yield) as a yellow solid. ¹H NMR (400 MHz, DMSO-d₆) δ=9.94 (s, 1H), 8.61 (d, J=8.8 Hz, 1H) 6.15 (t, J=8.0 Hz, 1H), 7.95-7.86 (m, 2H), 7.58 (d, J=8.8 Hz, 1H), 7.51-7.44 (m, 3H), 7.12 (dd, J=8.4, 3.2 Hz, 1H) 2.38 (s, 3H). ¹H NMR (376 MHz, DMSO-d₆) δ=−72.167.

Step 2. To a solution of N-(6-fluoro-2-methylpyridin-3-yl)-3-nitro-6-phenylpyridin-2-amine (200 mg, 0.622 mmol) in DMSO (4 mL) and methanol (0.5 mL), Na₂S₂O₄ (433 mg, 2.49 mmol) and 2-aminonicotinaldehyde (106 mg, 0.871 mmol) were added and the reaction stirred at 100° C. for 16 hrs. Water (40 mL) was added to the mixture and the whole extracted with EtOAc (20 mL×3). The organic layer was washed with brine then dried over Na₂SO₄ and concentrated in vacuum to give a crude. The crude was purified by flash column (0-80% of EtOAc in PE) to give 3-(3-(6-fluoro-2-methylpyridin-3-yl)-5-phenyl-3H-imidazo[4,5-b]pyridin-2-yl)pyridin-2-amine (180 mg, 73.5% yield) as a yellow oil. ¹H NMR (400 MHz, DMSO-d₆) δ=8.35-8.28 (m, 1H) 6.12 (t, J=8.0 Hz, 1H), 8.05-7.99 (m, 4H), 7.50-7.44 (m, 2H), 7.43-7.37 (m, 1H), 7.27 (dd, J=8.4, 3.2 Hz, 1H), 7.18 (dd, J=8.0, 2.0 Hz, 1H), 7.10 (s, 2H), 6.47 (dd, J=8.0, 4.8 Hz, 1H), 2.14 (s, 3H). ¹H NMR (376 MHz, DMSO-d₆) δ=−67.456.

Step 3. To a solution of 3-(3-(6-fluoro-2-methylpyridin-3-yl)-5-phenyl-3H-imidazo[4,5-b]pyridin-2-yl)pyridin-2-amine (190 mg, 0.479 mmol) in DMSO (4 mL), methyl 4-aminocyclohexane-1-carboxylate (139 mg, 0.719 mmol) and DIEA (0.25 mL, 1.44 mmol) were added and the reaction stirred at 120° C. for 16 hrs. Water (40 mL) was added and the whole extracted with EtOAc (30 mL×3). The organic layer was dried over Na₂SO₄ and concentrated in vacuo to provide the crude material. The crude was purified by flash column (0-60% of EtOAc in PE) to give methyl 4-((5-(2-(2-aminopyridin-3-yl)-5-phenyl-3H-imidazo[4,5-b]pyridin-3-yl)-6-methylpyridin-2-yl)amino)cyclohexane-1-carboxylate (40.0 mg, 15.6% yield) as a yellow oil. 1H NMR (400 MHz, DMSO-d₆) δ=8.26 (d, J=8.4 Hz, 1H), 8.06-7.90 (m, 4H), 7.55-7.35 (m, 4H), 7.04-7.31 (m, 3H), 6.78 (dd, J=18.0, 7.2 Hz, 1H), 6.53-6.40 (m, 2H), 3.65-3.57 (m, 3H), 2.24-2.02 (m, 2H), 1.96-1.92 (m, 1H), 1.88 (d, J=4.0 Hz, 3H), 1.81-1.22 (m, 7H).

Step 4. To a solution of methyl 4-((5-(2-(2-aminopyridin-3-yl)-5-phenyl-3H-imidazo[4,5-b]pyridin-3-yl)-6-methylpyridin-2-yl)amino)cyclohexane-1-carboxylate (40.0 mg, 0.0750 mmol) in THF (2 mL) and water (2 mL), LiOH·H₂O (6.30 mg, 0.150 mmol) was added and the reaction stirred at 25° C. for 4 hrs. The crude was purified by prep-HPLC (column: Welch Xtimate C18 150*30 mm*5 μm, mobile phase: water(FA)-ACN; B %: 20%-60%, 30 min) to give 4-((5-(2-(2-aminopyridin-3-yl)-5-phenyl-3H-imidazo[4,5-b]pyridin-3-yl)-6-methylpyridin-2-yl)amino)cyclohexane-1-carboxylic acid I-109 (17.0 mg, 41.5% yield) as a yellow solid. 1H NMR (400 MHz, DMSO-d₆) δ=8.26 (d, J=8.4 Hz, 1H), 8.05-7.99 (m, 3H), 7.96 (d, J=8.4 Hz, 1H), 7.52-7.44 (m, 2H), 7.43-7.37 (m, 2H), 7.28 (dd, J=1.6, 7.8 Hz, 1H), 7.22 (s, 1H), 6.76 (d, J=7.8 Hz, 1H), 6.51-6.39 (m, 2H), 3.74-3.58 (m, 1H), 2.20 (tt, J=3.6, 12.0 Hz, 1H), 2.09-1.92 (m, 4H), 1.89 (s, 3H), 1.51-1.37 (m, 2H), 1.32-1.15 (m, 2H). HPLC Rₜ=3.558 min in 8 min chromatography, purity 95.6%. LCMS Rₜ=1.449 min in 7 min chromatography, purity 96.0%, MS ESI calcd. For 519.24 [M+H]⁺ 520.24, found 520.0.

### Example 101. Synthesis of 3-((5-(2-(2-aminopyridin-3-yl)-6-phenyl-1H-benzo[d]imidazol-1-yl)pyridin-2-yl)carbamoyl)cyclopentane-1-carboxylic acid (Compound I-110)

### Synthesis of methyl 3-(chlorocarbonyl)cyclopentane-1-carboxylate

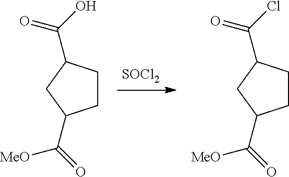

Step 1. A mixture of 3-(methoxycarbonyl)cyclopentane-1-carboxylic acid (150 mg, 0.871 mmol) in SOCl₂ (2.0 mL, 0.537 mmol) was stirred at 80° C. for 2 hrs. The reaction mixture was concentrated to give methyl 3-(chlorocarbonyl)cyclopentane-1-carboxylate (100 mg, 78.4% yield) as colorless oil.

### Synthesis of 3-((5-(2-(2-aminopyridin-3-yl)-6-phenyl-1H-benzo[d]imidazol-1-yl)pyridin-2-yl)carbamoyl)cyclopentane-1-carboxylic acid (Compound I-110)

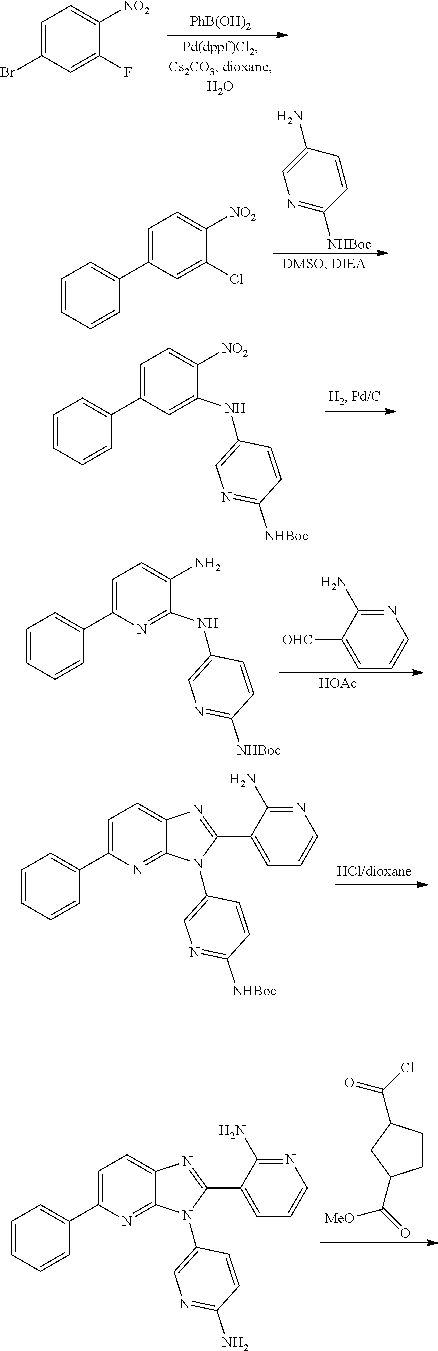

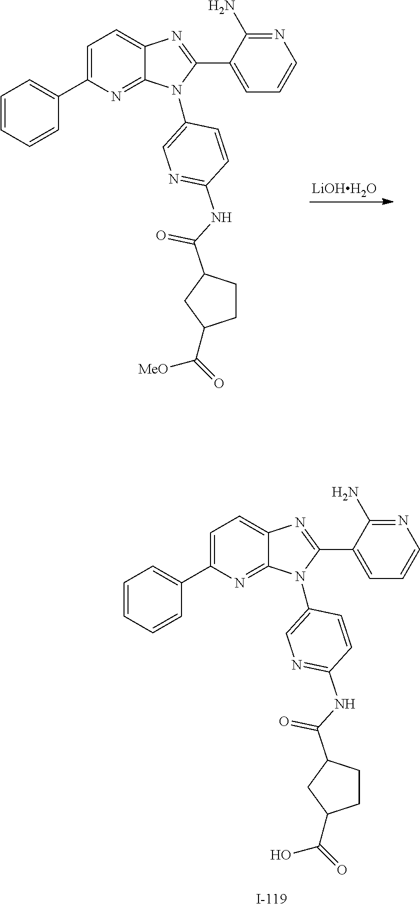 [SMILES: COC(=O)C1CCC(C(=O)Nc2ccc(-n3c(-c4cccnc4N)nc4ccc(-c5ccccc5)nc43)cn2)C1 ; "LiOH·H2O" ; Nc1ncccc1-c1nc2ccc(-c3ccccc3)nc2n1-c1ccc(NC(=O)C2CCC(C(=O)O)C2)nc1]

### Synthesis of 3-((5-(2-(2-aminopyridin-3-yl)-6-phenyl-1H-benzo[d]imidazo-1-yl)pyridin-2-yl)carbamoyl)cyclopentane-1-carboxylic acid (Compound I-110)

Step 3. To a solution of tert-butyl N-[5-(2-nitro-5-phenyl-anilino)-2-pyridyl]carbamate (2.65 g, 6.52 mmol) in methanol (20.0 mL), Pd/C (2.74 g, wet, purity 10%) was added and the reaction mixture was degassed and purged with H₂ (×3). The mixture was stirred at 25° C. for 12 hrs under H₂ (15 psi). The mixture was filtered through celite and washed with solvents (MeOH: DCM=1:5, 50 mL×4). The filtrate was concentrated to give tert-butyl (5-((4-amino-[1,1′-biphenyl]-3-yl)amino)pyridin-2-yl)carbamate (1.84 g, 74.9% yield) as a yellow solid. LCMS Rₜ=0.547 min in 1 min chromatography, purity 32.2%, MS ESI calcd. for 376.19 [M+H]⁺ 377.19, found 377.15.

Step 4. To a solution of tert-butyl (5-((4-amino-[1,1′-biphenyl]-3-yl)amino)pyridin-2-yl)carbamate (1.84 g, 4.89 mmol) in acetic acid (10.0 mL), 2-aminonicotinaldehyde (649 mg, 5.31 mmol) was added under N₂ and the mixture was stirred at 100° C. for 12 hrs. The reaction mixture was concentrated. The residue was purified by flash silica gel chromatography (eluent with 0-43% of EtOAc in PE) to give tert-butyl (5-(2-(2-aminopyridin-3-yl)-6-phenyl-1H-benzo[d]imidazol-1-yl)pyridin-2-yl)carbamate (1.20 g, 46.7% yield) as a yellow solid. LCMS Rₜ=0.462 min in 1 min chromatography, purity 77.4%, MS ESI calcd. for 478.21 [M+H−100]⁺ 379.21, found 379.2.

Step 5. A mixture of tert-butyl (5-(2-(2-aminopyridin-3-yl)-6-phenyl-IH-benzo[d]imidazol-1-yl)pyridin-2-yl)carbamate (1.20 g, 2.51 mmol) in HCl/dioxane (10.0 mL, 2.51 mmol) and methanol (2.0 mL) was stirred at 25° C. for 12 hrs. The reaction mixture was concentrated directly. The mixture was purified by prep-HPLC (Welch Ultimate XB-CN 250*50*10 um; mobile phase: [Heptane-EtOH (0.1% NH₃H₂O)]; B %:15%-45%, 15 min) to give 5-(2-(2-aminopyridin-3-yl)-6-phenyl-1H-benzo[d]imidazol-1-yl)pyridin-2-amine (0.140 g, 14.8% yield) as a yellow solid. ¹H NMR (400 MHz, CDCl₃) δ=8.17 (d, J=2.0 Hz, 1H), 8.07-8.05 (m, 1H), 7.89-7.85 (m, 1H), 7.63-7.56 (m, 4H), 7.46-7.40 (m, 3H), 7.37-7.34 (m, 2H), 7.34-7.33 (m, 1H), 7.22 (dd, J=1.6, 7.6 Hz, 1H), 6.65-6.57 (m, 3H), 6.44 (dd, J=4.8, 7.6 Hz, IH). LCMS Rₜ=0.432 min in 1 min chromatography, purity 74.2%, MS ESI calcd. for 378.16 [M+H]⁺ 379.16, found 379.1.

Step 6. To a solution of 5-(2-(2-aminopyridin-3-yl)-6-phenyl-1H-benzo[d]imidazol-1-yl)pyridin-2-amine (1.00 g, 60.0 mmol) in pyridine (3.0 mL), methyl 3-(chlorocarbonyl)cyclopentane-1-carboxylate (100 mg, 0.523 mmol) was added and the mixture was stirred at 50° C. for 1 hr. The reaction mixture was concentrated. Water (20.0 mL) was added and the resulting mixture was extracted with EtOAc (20 mL×3). The combined organic phase was washed with brine (20 mL), dried over anhydrous Na₂SO₄, filtered and concentrated. Methyl 3-[[5-[2-(2-amino-3-pyridyl)-6-phenyl-benzimidazol-1-yl]-2-pyridyl]carbamoyl]cyclopentanecarboxylate (70.0 mg, 49.9% yield) was obtained as a yellow solid. LCMS Rₜ=0.612 min in 1 min chromatography, purity 22.6%. MS ESI calcd. for 532.22 [M+H]⁺ 533.22, found 533.2.

Step 7. To a solution of methyl 3-((5-(2-(2-aminopyridin-3-yl)-6-phenyl-IH-benzo[d]imidazol-1-yl)pyridin-2-yl)carbamoyl)cyclopentane-1-carboxylate (60.0 mg, 0.113 mmol) in THF (2.0 mL) and water (2.0 mL), LiOH·H₂O (45.0 mg, 1.13 mmol) was added and the mixture was stirred at 25° C. for 12 hrs. The reaction mixture was concentrated directly. The residue was purified by prep-HPLC (Column. Welch Xtimate C18 40*200 mm 7 μm; mobile phase: [water(FA)-ACN]; B %: 4%-44%; 25 min) and prep-HPLC (Column: Welch Xtimate C18 40*200 mm 7 um; mobile phase: [water(NH₄HCO₃)-ACN]; B %: 2%-42%; 25 min) to afford 3-((5-(2-(2-aminopyridin-3-yl)-6-phenyl-1H-benzo[d]imidazol-1-yl)pyridin-2-yl)carbamoyl)cyclopentane-1-carboxylic acid I-110 (1.00 mg, 1.69% yield) as a white solid. ¹H NMR (400 MHz, DMSO-d₆) δ=10.98-10.77 (m, 1H), 8.39-8.33 (m, 1H), 8.31-8.27 (m, 1H), 8.03-7.96 (m, 2H), 7.92-7.86 (m, 1H), 7.70-7.63 (m, 3H), 7.47-7.40 (m, 3H), 7.37-7.31 (m, 1H), 7.28-7.23 (m, 1H), 6.91 (s, 2H), 6.50-6.45 (m, 1H), 3.03-2.97 (m, 1H), 2.78-2.69 (m, 1H), 2.19-2.10 (m, 1H), 2.02-1.94 (m, 1H), 1.91-1.78 (m, 4H). HPLC Rₜ=2.582 min in 8 min chromatography, purity 98.5% LCMS Rₜ=1.222 min in 2 min chromatography, purity 93.3%, MS ESI calcd. for 518.21 [M+H]⁺ 519.21, found 519.1.

### Example 102. Synthesis of 3-((5-(2-(2-aminopyridin-3-yl)-6-phenyl-1H-benzo[d]imidazol-1-yl)pyridin-2-yl)carbamoyl)cyclohexane-1-carboxylic acid (Compound I-111)

### Synthesis of methyl 3-(chlorocarbonyl)cyclohexane-1-carboxylate

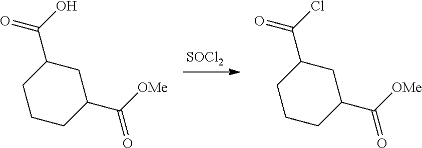 [SMILES: COC(=O)C1CCCC(C(=O)O)C1 ; "SOCl2" ; COC(=O)C1CCCC(C(=O)Cl)C1]

Step 1. A mixture of 3-(methoxycarbonyl) cyclohexane-1-carboxylic acid (100 mg, 0.537 mmol) in SOCl₂ (2.0 mL, 0.537 mmol) was stirred at 80° C. for 2 hrs. The reaction mixture was concentrated to give methyl 3-(chlorocarbonyl)cyclohexane-1-carboxylate (100 mg, 90.9% yield) as a colorless oil.

### Synthesis of 3-((S-(2-(2-aminopyridin-3-yl)-6-phenyl-IH-benzo[d]imidazol-1-yl)pyridin-2-yl)carbamoyl)cyclohexane-1-carboxylic acid (Compound I-111)

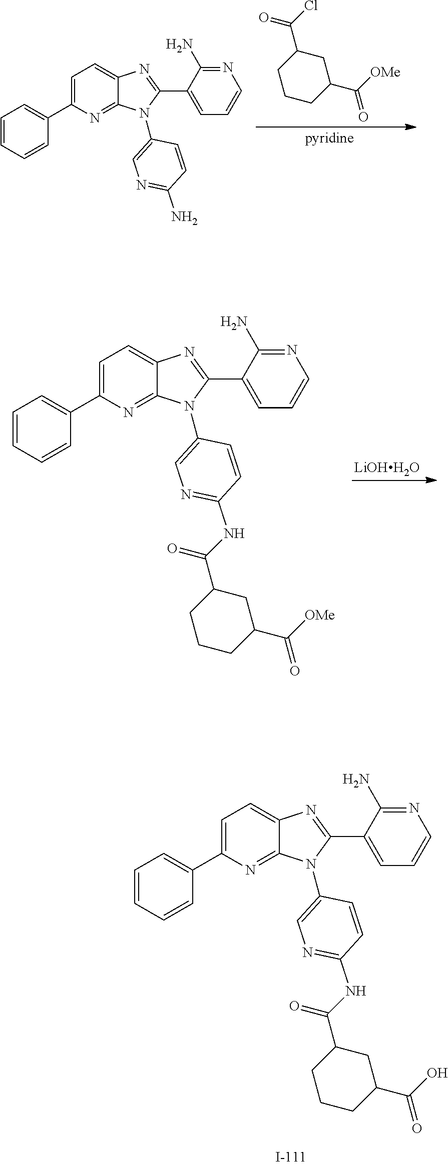

Step 1. To a solution of 5-(2-(2-aminopyridin-3-yl)-6-phenyl-1H-benzo[d]imidazol-1-yl)pyridin-2-amine (1.00 g, 50.0 mmol) in pyridine (3.0 mL), methyl 3-(chlorocarbonyl)cyclohexane-1-carboxylate (95.0 mg, 0.462 mmol) was added and the mixture was stirred at 50° C. for 1 hr. The reaction mixture was concentrated. Water (20.0 mL) was added, and the resulting mixture was extracted with EtOAc (20 mL×3). The combined organic phase was washed with brine (20.0 mL), dried over anhydrous Na₂SO₄, filtered and concentrated to give methyl 3-((5-(2-(2-aminopyridin-3-yl)-6-phenyl-1H-benzo[d]imidazol-1-yl)pyridin-2-yl)carbamoyl)cyclohexane-1-carboxylate (50 mg, 46.0% yield) as a yellow solid. LCMS Rₜ=0.632 min in 1 min chromatography, purity 13.6%, MS ESI calcd. for 546.24 [M+H]⁺ 547.24, found 547.3.

Step 2. To a solution of methyl 3-((5-(2-(2-aminopyridin-3-yl)-6-phenyl-1H-benzo[d]imidazol-1-yl)pyridin-2-yl)carbamoyl)cyclohexane-1-carboxylate (50.0 mg, 0.0915 mmol) in THF (2.0 mL) and water (2.0 mL), LiOH·H₂O (38.0 mg, 0.915 mmol) was added and the mixture was stirred at 25° C. for 12 hrs. NaOH (37 mg, 0.915 mmol) was then added and the mixture stirred for 12 hrs. The reaction mixture was concentrated directly. The residue was purified by prep-HPLC (Welch Xtimate C18 40*200 mm 7 μm; mobile phase: [water(HCl)-ACN]; B %: 2%-42%; 30 min) and prep-HPLC (Welch Xtimate C18 40*200 mm 7 μm; mobile phase: [water(FA)-ACN]; B %: 6%-46%; 25 min) to afford 3-((5-(2-(2-aminopyridin-3-yl)-6-phenyl-1H-benzo[d]imidazol-1-yl)pyridin-2-yl)carbamoyl)cyclohexane-1-carboxylic acid I-111 (1.00 mg, 1.69% yield) as a yellow solid. ¹H NMR (400 MHz, DMSO-d₆) δ=10.69 (s, 1H), 8.36 (d, J=2.4 Hz, 1H), 8.28 (d, J=8.8 Hz, 1H), 8.02-7.97 (m, 2H), 7.88 (d, J=8.4 Hz, 1H), 7.69-7.63 (m, 3H), 7.47-7.40 (m, 3H), 7.37-7.31 (m, 1H), 7.26 (dd, J=1.6, 7.6 Hz, 1H), 6.90 (s, 2H), 6.47 (dd, J=4.8, 7.6 Hz, H), 2.62-2.56 (m, 1H), 2.29-2.15 (m, 1H), 2.03 (d, J=12.6 Hz, 1H), 1.94-1.77 (m, 3H), 1.52-1.40 (m, 1H), 1.36-1.21 (m, 3H). HPLC Rₜ=2.713 min in 8 min chromatography, purity 99.6%. LCMS Rₜ=1.277 min in 2 min chromatography, purity 95.2%, MS ESI calcd. for 532.22 [M+H]⁺ 533.22, found 533.0.

### Example 103. Synthesis of 6-((5-(2-(2-aminopyridin-3-yl)-5-phenyl-3H-imidazo[4,5-b]pyridin-3-yl)pyridin-2-yl)carbamoyl)spiro[33]heptane-2-carboxylic acid (Compound I-112)

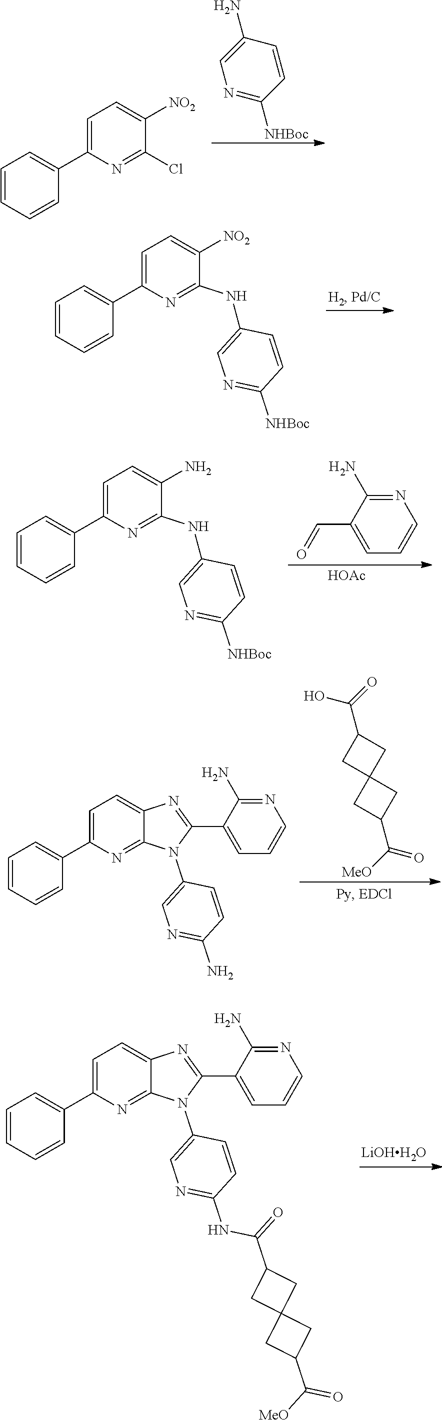

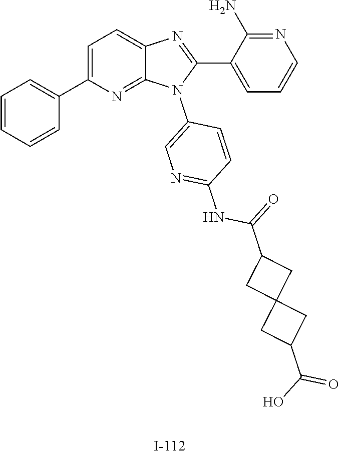 [SMILES: Nc1ncccc1-c1nc2ccc(-c3ccccc3)nc2n1-c1ccc(NC(=O)C2CC3(CC(C(=O)O)C3)C2)nc1]

Step 1. To a solution of 2-chloro-3-nitro-6-phenylpyridine (1.70 g, 7.25 mmol) in DMSO (15 mL), tert-butyl (5-aminopyridin-2-yl)carbamate (1.82 g, 8.69 mmol) and DIEA (2.81 g, 21.7 mmol) were added at 25° C. and the mixture was stirred at 80° C. for 14 hrs. EtOAc (20 mL) was added to the reaction and the resulting suspension was washed with saturated aqueous LiCl (20 mL×3), dried over anhydrous Na₂SO₄, filtered and concentrated. Isopropyl ether (20 mL) was added, and the mixture stirred for 10 mins. The reaction mixture was filtered and the filter cake was concentrated to give tert-butyl (5-((3-nitro-6-phenylpyridin-2-yl)amino)pyridin-2-yl)carbamate (3.80 g, crude) as a red solid. ¹H NMR (CDCl₃, 400 MHz) δ_{H}=10.18 (s, 1H), 8.65-8.55 (m, 2H), 8.18 (d, J=9.2 Hz, 1H), 8.09-7.98 (m, 3H), 7.66-7.58 (m, 1H), 7.52 (s, 3H), 7.36 (d, J=8.4 Hz, 1H), 1.57 (s, 9H).

Step 2. To a mixture of tert-butyl (5-((3-nitro-6-phenylpyridin-2-yl)amino)pyridin-2-yl)carbamate (2.50 g, 9.68 mmol) in THF (20 mL), Pd/C (500 mg, 10%) was added under the Ar atmosphere. The suspension was degassed and purged with H₂ (×3) and the mixture was stirred under H₂ (15 psi) at 25° (for 14 hrs. The mixture was filtered via a celite pad and the pad washed with (DCM:MeOH=1:1, 150 mL×5). The filtrate was concentrated to give tert-butyl (5-((3-amino-6-phenylpyridin-2-yl)amino)pyridin-2-yl)carbamate (2.00 g, 79.4% yield) as a yellow solid, which was used in the next step without further purification. ¹H NMR (DMSO-d₆, 400 MHz) δ_{H}=9.53 (s, 1H), 8.74 (d, J=2.4 Hz, 1H), 8.10 (dd, J=2.8, 9.2 Hz, 1H), 7.98 (s, IH), 7.90 (dd, J=1.6, 8.8 Hz, 2H), 7.73 (d, J=9.2 Hz, 1H), 7.40 (t, J=7.6 Hz, 2H), 7.29-7.22 (m, 2H), 6.98 (d, J=7.6 Hz, 1H), 5.28 (s, 2H), 1.48 (s, 9H).

Step 3. To a solution of tert-butyl (5-((3-amino-6-phenylpyridin-2-yl)amino)pyridin-2-yl)carbamate (2.00 g, 5.30 mmol) in acetic acid (20 mL), 2-aminopyridine-3-carbaldehyde (777 mg, 6.36 mmol) was added at 25° C. and the mixture was stirred at 100° C. for 24 hrs. The reaction was concentrated. The residue was purified by Combi-Flash column (EtOAc in PE=0%-100%) to give 3-(3-(6-aminopyridin-3-yl)-5-phenyl-3H-imidazo[4,5-b]pyridin-2-yl)pyridin-2-amine (320 mg, 15.9% yield) as a yellow solid. LCMS Rₜ=0.938 min in 2.5 min chromatography, purity 60.3%, MS ESI calcd. for 379.15, [M+H]⁺ 380.15, found 380.1.

Step 4. To a solution of 3-(3-(6-aminopyridin-3-yl)-5-phenyl-3H-imidazo[4,5-b]pyridin-2-yl)pyridin-2-amine (100 mg, 0.264 mmol) and 6-(methoxycarbonyl)spiro[3.3]heptane-2-carboxylic acid (52.0 mg, 0.264 mmol) in pyridine (2 mL), EDCI (76 mg, 0.395 mmol) was added and the mixture was stirred at 25° C. for 12 hrs. Water (5 mL) was added to the reaction mixture and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent of 0-5% MeOH in DCM) to give methyl 6-((5-(2-(2-aminopyridin-3-yl)-5-phenyl-3H-imidazo[4,5-b]pyridin-3-yl)pyridin-2-yl)carbamoyl)spiro[3.3]heptane-2-carboxylate (23.0 mg, 15.6% yield) as a yellow oil. ¹H NMR (DMSO-d₆, 400 MHz) δ_{H}=8.62-8.17 (m, 3H), 8.04-7.98 (m, 3H), 7.93-7.82 (m, 3H), 7.60-7.38 (m, 5H), 7.29-7.26 (m, 1H), 6.70-6.54 (m, 1H), 3.68 (s, 3H), 3.17-3.02 (m, 2H), 2.44-2.29 (m, 8H).

Step 5. To a solution of methyl 6-((5-(2-(2-aminopyridin-3-yl)-5-phenyl-3H-imidazo[4,5-b]pyridin-3-yl)pyridin-2-yl)carbamoyl)spiro[3.3]heptane-2-carboxylate (20.0 mg, 0.0357 mmol) in THF (1 mL) and water (1 mL), LiOH·H₂O (1.5 mg, 0.0357 mmol) was added and the mixture was stirred at 25° C. for 2 hrs. The reaction mixture was concentrated directly. The residue was purified by prep-HPLC (Column: Welch Xtimate C18 150*30 mm*5 μm; mobile phase: [water (FA)-ACN]; B %: 0%-35%; 25 min) to give 6-((5-(2-(2-aminopyridin-3-yl)-5-phenyl-3H-imidazo[4,5-b]pyridin-3-yl)pyridin-2-yl)carbamoyl)spiro[3.3]heptane-2-carboxylic acid 1-112 (1.60 mg, 8.21% yield) as a light yellow solid. ¹H NMR (DMSO-d₆, 400 MHz) δ_{H}=10.55 (s, 1H), 8.44-8.23 (m, 3H), 8.08-7.93 (m, 5H), 7.51-7.31 (m, 4H), 6.84 (s, 2H), 6.51 (dd, J=4.8, 7.6 Hz, 1H), 3.25-3.22 (m, 1H), 2.97-2.87 (m, 1H), 2.30-2.08 (m, 8H). HPLC Rₜ=2.602 min in 8 min chromatography, purity 100%. LCMS Rₜ=1.206 min in 2 min chromatography, purity 97.7%, MS ESI calcd. for 545.22, [M+H]⁺ 546.22, found 546.0.

### Example 104. Alternate synthesis of methyl (1r,4r)-4-((5-(2-(2-aminopyridin-3-yl)-5-phenyl-3H-imidazo[4,5-b]pyridin-3-yl)pyridin-2-yl)carbamoyl)cyclohexane-1-carboxylate (Compound I-113)

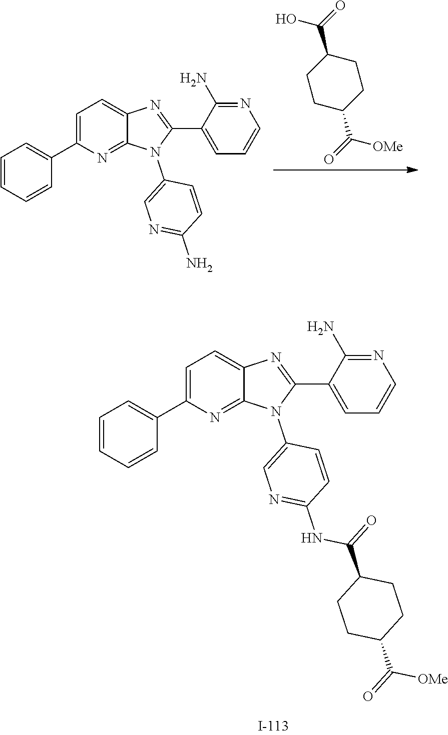 [SMILES: Nc1ccc(-n2c(-c3cccnc3N)nc3ccc(-c4ccccc4)nc32)cn1 ; COC(=O)[C@H]1CC[C@H](C(=O)O)CC1 ; COC(=O)[C@H]1CC[C@H](C(=O)Nc2ccc(-n3c(-c4cccnc4N)nc4ccc(-c5ccccc5)nc43)cn2)CC1]

Step 1. To a mixture of 3-(3-(6-aminopyridin-3-yl)-5-phenyl-3H-imidazo[4,5-b]pyridin-2-yl)pyridin-2-amine (50.0 mg, 0.130 mmol) in pyridine (2.00 mL), (1r,4r)-4-(methoxycarbonyl)cyclohexane-1-carboxylic acid (37.0 mg, 0.190 mmol) and EDCI (76.0 mg, 0.390 mmol) were added and the mixture was stirred at 55° C. for 2 hrs. Water (5 mL) was added and the resulting mixture was extracted with EtOAc (15 mL×3) and washed with brine (20 mL×2), dried over anhydrous Na₂SO₄, filtered and concentrated. The residue was purified by prep-HPLC (condition: water (FA)-ACN; Column: Xtimate C18 40*200 mm*7 μm; B %: 12%-52%; Gradient Time, 25 min) to afford methyl (1r,4r)-4-((5-(2-(2-aminopyridin-3-yl)-5-phenyl-3H-imidazo[4,5-b]pyridin-3-yl)pyridin-2-yl)carbamoyl)cyclohexane-1-carboxylate I-113 (10.3 mg, 14.0% yield) as a white solid. ¹H NMR (400 MHz, DMSO-d₆) δ=10.68 (s, 1H), 8.43 (d, J=2.4 Hz, 1H), 8.27 (dd, J=8.4, 13.6 Hz, 2H), 8.09-8.00 (m, 4H), 7.97 (dd, J=2.8, 9.2 Hz, 1H), 7.52-7.45 (m, 2H), 7.42 (d, J=7.2 Hz, 1H), 7.37 (dd, J=2.0, 8.0 Hz, 1H), 6.85 (s, 2H), 6.52 (dd, J=4.4, 7.6 Hz, 1H), 3.61 (s, 3H), 2.54-2.53 (m, 1H), 2.36-2.30 (m, 1H), 2.03-1.88 (m, 4H), 1.56-1.43 (m, 2H), 1.43-1.31 (m, 2H). HPLC Rₜ=2.949 min in 8 min chromatography, purity 98.1%. LCMS Rₜ=1.950 min in 4 min chromatography, purity 99.2%, MS ESI calcd. for 547.23 [M+H]⁺ 548.23, found 548.3.

### Example 105. Synthesis of (1s,4s)-4-(((5-(2-(2-aminopyridin-3-yl)-5-phenyl-3H-imidazo[4,5-b]pyridin-3-yl)pyridin-2-yl)amino)methyl)cyclohexane-1-carboxylic acid (Compound I-114)

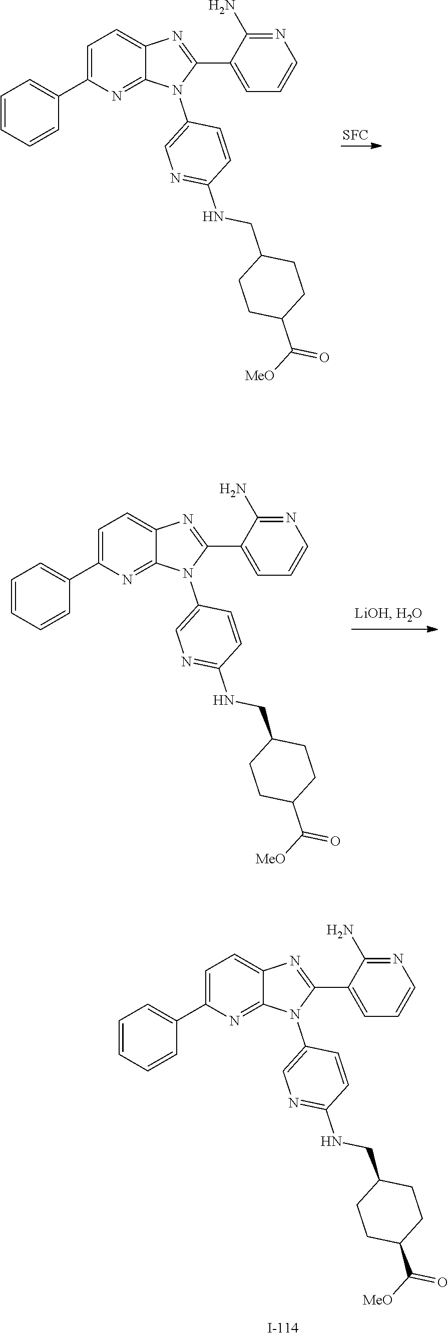

Step 1. Methyl 4-(((5-(2-(2-aminopyridin-3-yl)-5-phenyl-3H-imidazo[4,5-b]pyridin-3-yl)pyridin-2-yl)amino)methyl)cyclohexane-1-carboxylate was purified by SFC (column: DAICEL CHIRALPAK AD 250 mm*30 mm, 10 μm; mobile phase: CO₂-i-POH; B %:50%-50%, 150 min) to give methyl (1s,4s)-4-(((5-(2-(2-aminopyridin-3-yl)-5-phenyl-3H-imidazo[4,5-b]pyridin-3-yl)pyridin-2-yl)amino)methyl)cyclohexane-1-carboxylate (200 mg, 71.7% yield) as a red solid. ¹H NMR (400 MHz, DMSO-d₆) δ=8.24 (d, J=8.4 Hz, 1H), 8.10-8.00 (m, 4H), 7.96 (d, J=8.4 Hz, 1H), 7.52-7.44 (m, 3H), 7.43-7.35 (m, 2H), 6.99-6.89 (m, 3H), 6.59 (d, J=8.4 Hz, 1H), 6.49 (dd, J=4.8, 7.6 Hz, 1H), 3.61 (s, 3H), 3.21-3.13 (m, 2H), 2.60-2.56 (m, 1H), 1.97-1.83 (m, 2H), 1.71-1.48 (m, 5H), 1.30-1.19 (m, 2H).

Step 2. To a solution of methyl (1s,4s)-4-(((5-(2-(2-aminopyridin-3-yl)-5-phenyl-3H-imidazo[4,5-b]pyridin-3-yl)pyridin-2-yl)amino)methyl)cyclohexane-1-carboxylate (200 mg, 0.375 mmol) in THE (1 mL) and water (1 mL), LiOH·H₂O (63.0 mg, 1.50 mmol) was added and the reaction stirred at 25° C. for 16 hrs. The crude was purified by prep-HPLC (column: Welch Xtimate C18 150*30 mm*5 μm; mobile phase:water(FA)-ACN; B %: 20%-60%, 30 min) to give (1s,4s)-4-(((5-(2-(2-aminopyridin-3-yl)-5-phenyl-3H-imidazo[4,5-b]pyridin-3-yl)pyridin-2-yl)amino)methyl)cyclohexane-1-carboxylic acid 1-114 (72.0 mg, 36.7% yield) as a yellow solid. LCMS Rₜ=2.721 min in 4 min chromatography, purity 100%, MS ESI calcd. For 519.24 [M+H]⁺ 520.24, found 520.1. HPLC Rₜ=4.364 min in 8 min chromatography, purity 99.6%. ¹H NMR (400 MHz, DMSO-d₆) δ=12.16-11.96 (m, 1H), 8.24 (d, J=8.4 Hz, 1H), 8.07-8.00 (m, 4-i), 7.96 (d, J=8.4 Hz, 1Hz), 7.51-7.44 (m, 3H), 7.43-7.36 (m, 2H), 7.01-6.89 (m, 3H), 6.59 (d, J=8.8 Hz, 1H), 6.50 (dd, J=4.4, 7.6 Hz. 1H), 3.17 (t, J=6.0 Hz, 2H), 2.48-2.44 (m, 1H), 1.96-1.82 (m, 2H), 1.75-1.57 (m, 3H), 1.55-1.44 (m, 2H), 1.35-1.15 (m, 2H).

### Example 106. Synthesis of (1r,4r)-N-(5-(2-(2-aminopyridin-3-yl)-5-phenyl-3H-imidazo[4,5-b]pyridin-3-yl)pyridin-2-yl)cyclohexane-1,4-dicarboxamide (Compound I-115)

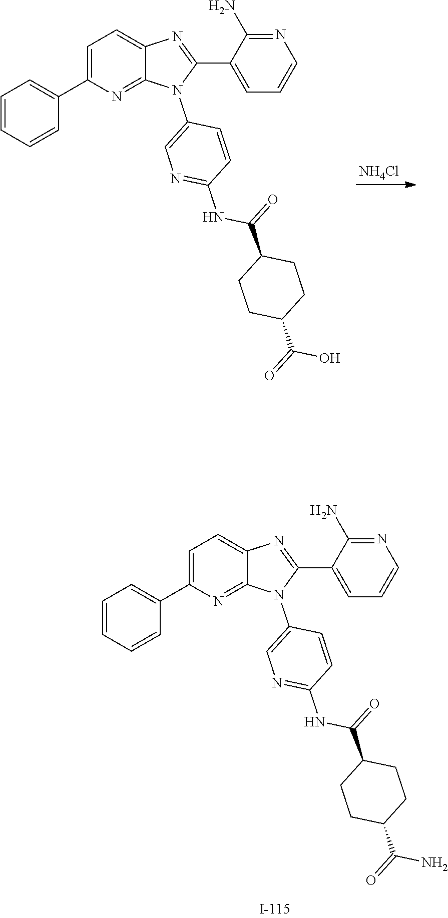 [SMILES: Nc1ncccc1-c1nc2ccc(-c3ccccc3)nc2n1-c1ccc(NC(=O)[C@H]2CC[C@H](C(=O)O)CC2)nc1 ; "NH4Cl" ; Nc1ncccc1-c1nc2ccc(-c3ccccc3)nc2n1-c1ccc(NC(=O)[C@H]2CC[C@H](C(N)=O)CC2)nc1]

Step 1. To a mixture of (1r,4r)-4-((5-(2-(2-aminopyridin-3-yl)-5-phenyl-3H-imidazo[4,5-b]pyridin-3-yl)pyridin-2-yl)carbamoyl)cyclohexane-1-carboxylic acid (80.0 mg, 0.150 mmol) in DMF (2.00 mL), DIPEA (97.0 mg, 0.750 mmol) and NH₄Cl (12.0 mg, 0.220 mmol) were added and the mixture was stirred at 25° C. for 10 mins. HATU (114 mg, 0.300 mmol) was then added and the mixture was stirred at 25° C. for 2 hrs. The reaction mixture was filtered. The residue was purified by prep-HPLC (condition: water (NH₄HCO₃)-ACN; Column: Xtimate C18 40*200 mm*7 μm; B %: 16%-56%; Gradient Time, 25 min) to afford (1r,4r)-N-(5-(2-(2-aminopyridin-3-yl)-5-phenyl-3H-imidazo[4,5-b]pyridin-3-yl)pyridin-2-yl)cyclohexane-1,4-dicarboxamide 1-115 (35.0 mg, 43.7% yield) as a white solid. ¹H NMR (400 MHz, DMSO-d₆) δ=10.69 (s, 1H), 8.43 (d, J=2.8 Hz, 1H), 8.32-8.23 (m, 2H), 8.06-8.00 (m, 4H), 7.96 (dd, J=2.8, 9.2 Hz, 1H), 7.48 (t, J=7.2 Hz, 2H), 7.44-7.36 (m, 2H), 7.24 (s, 1H), 6.85 (s, 2H), 6.71 (s, 1H), 6.52 (dd, J=4.8, 7.6 Hz, 1H), 2.54-2.53 (m, 1H), 2.16-2.06 (m, 1H), 1.95-1.77 (m, 4H), 1.51-1.30 (m, 4H). HPLC Rₜ=2 459 min in 8 min chromatography, purity 99.8%. LCMS Rₜ=1.497 min in 4 min chromatography, purity 99.9%, MS ESI calcd. for 532.23 [M+H]⁺ 533.23, found 533.3.

### Example 107. Synthesis of (1r,4r)-4-((5-(2-(2-aminopyridin-3-yl)-5-(1-methyl-1H-pyrazol-5-yl)-3H-imidazo[4,5-b]pyridin-3-yl)pyridin-2-yl)carbamoyl)cyclohexane-1-carboxylic acid (Compound I-116)

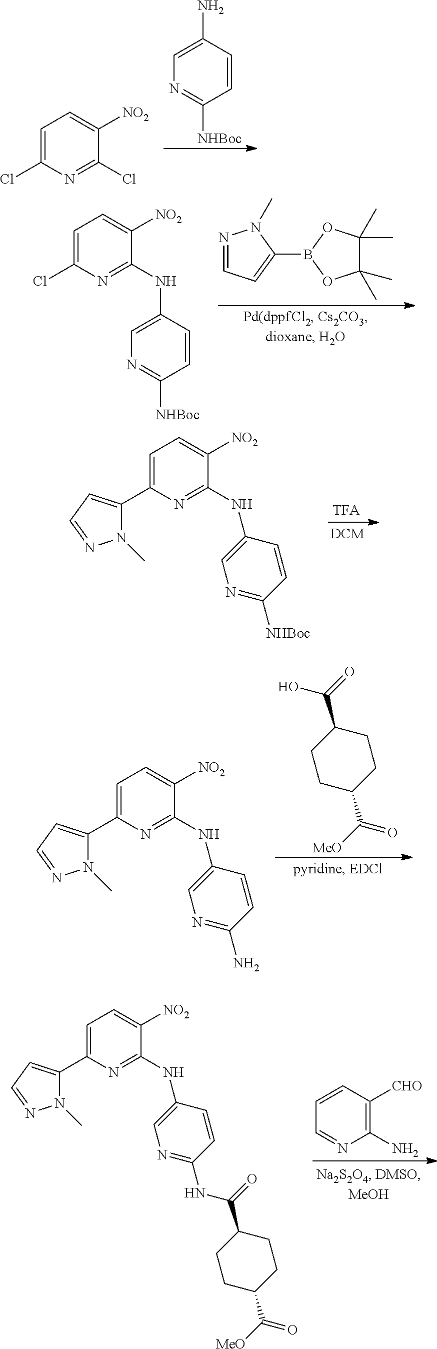

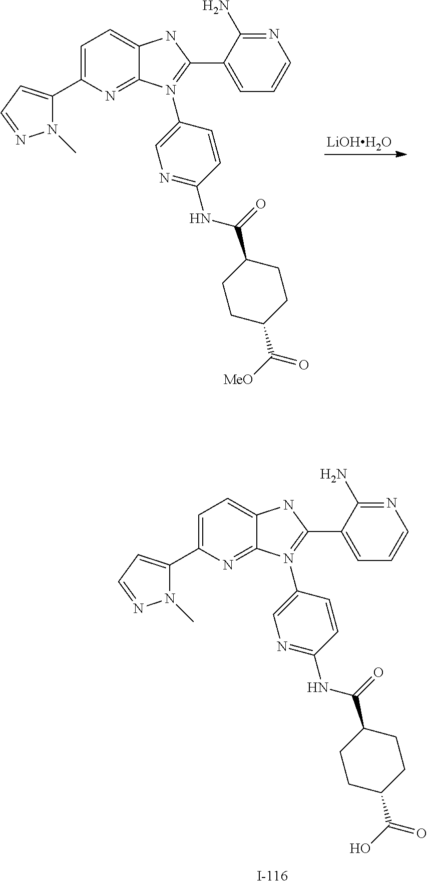

Step 1. To a solution of 2,6-dichloro-3-nitropyridine (5.00 g, 25.9 mmol) in ethanol (50 mL), NaHCO₃ (2.62 g, 25.9 mmol) and tert-butyl (5-aminopyridin-2-yl)carbamate (4.88 g, 23.3 mmol) were added at 25° C. and the mixture was stirred at 40° C. for 16 hrs. The reaction mixture was concentrated directly. Water (20 mL) was then added to the residue, and the resulting mixture was stirred at 25° C. for 2 hrs. The resulting mixture was filtered. The filter cake was stirred in ACN (20 mL) at 25° C. for 2 hrs. Then resulting mixture was filtered. The filter cake was concentrated to give tert-butyl (5-((6-chloro-3-nitropyridin-2-yl)amino)pyridin-2-yl)carbamate (7.00 g, 57.2% yield) as a red solid. ¹H NMR (DMSO-d₆, 400 MHz) δ_{H}=10.11 (s, 1H), 9.80 (s, 1H), 8.53 (d, J=8.8 Hz, 1H), 8.40 (d, J=2.4 Hz, 1H), 7.91-7.84 (m, 1H), 7.83-7.77 (m, 1H), 6.99 (d, J=8.8 Hz, 1H), 1.48 (s, 9H).

Step 2. To a solution of tert-butyl (5-((6-chloro-3-nitropyridin-2-yl)amino)pyridin-2-yl)carbamate (2.00 g, 5.47 mmol) in 1,4-dioxane (20 mL) and water (5 mL), l-methyl-5-(4,4,5,5-tetramethyl-1,3,2-dioxaborolan-2-yl)-1H-pyrazole (1.48 g, 7.11 mmol), Cs₂CO₃ (5.34 g, 16.4 mmol) and Pd(dppf)Cl₂ (200 mg, 0.27 mmol) were added and the mixture was stirred at 110° C. for 12 hrs. The reaction mixture was filtered and the filtrate was dissolved in water (100 mL). The resulting suspension was extracted with DCM (150 mL×3). The combined organic phase was washed with brine (80 mL), dried over anhydrous Na₂SO₄, filtered and concentrated. The residue was purified by Combi-Flash column (EtOAc in PE=0%-45%) to afford tert-butyl (5-((6-(1-methyl-1H-pyrazol-5-yl)-3-nitropyridin-2-yl)amino)pyridin-2-yl)carbamate (1.65 g, 73.4% yield) as a yellow solid. ¹H NMR (DMSO-d₆, 400 MHz) δ_{H}=10.04 (s, 1H), 9.81 (s, 1H), 8.57 (d, J=8.4 Hz, 1H), 8.35 (d, J=2.0 Hz, 1H), 7.90-7.78 (m, 2H), 7.50 (d, J=2.0 Hz, 1H), 7.36 (d, J=8.4 Hz, 1H), 7.02 (d, J=2.0 Hz, 1H), 3.72 (s, 3H), 1.48 (s, 9H).

Step 3. A solution of tert-butyl (5-((6-(1-methyl-IH-pyrazol-5-yl)-3-nitropyridin-2-yl)amino)pyridin-2-yl)carbamate (1.60 g, 3.89 mmol) in TFA (6.0 mL) and DCM (12 mL) was stirred at 25° C. for 1 hr. The reaction was concentrated directly. The resulting mixture was dissolved in EtOAc (10 mL) and adjusted to pH ~7 by aqueous NaOH (2 M). The resulting suspension was filtered. The filter cake was dried directly to afford N⁵-(6-(1-methyl-1H-pyrazol-5-yl)-3-nitropyridin-2-yl)pyridine-2,5-diamine (870 mg, 71.9% yield) as a red solid. LCMS Rₜ=0.478 min in 1.5 min chromatography, purity 87.3%, MS ESI calcd. for 311.11 [M+H]⁺ 312.11. found 312.1.

Step 4. To a solution of N-(6-(1-methyl-IH-pyrazol-5-yl)-3-nitropyridin-2-yl)pyridine-2,5-diamine (830 mg, 2.67 mmol) in pyridine (8 mL), (1r,4r)-4-(methoxycarbonyl)cyclohexane-1-carboxylic acid (596 mg, 3.20 mmol) and EDCI (1.53 g, 8.00 mmol) were added at 25° C. and the mixture was stirred at 80° C. for 2 hrs. The mixture was diluted with EtOAc (20 mL) and washed with aqueous HCl (10 mL×4, 1 M). The combined organic phase was washed with brine (20 mL×2), dried over anhydrous Na₂SO₄, filtered and concentrated. The residue was purified by Combi-Flash column (EtOAc in PE=0%-60%) to afford methyl (1r,4r)-4-((5-((6-(1-methyl-1H-pyrazol-5-yl)-3-nitropyridin-2-yl)amino)pyridin-2-yl)carbamoyl)cyclohexane-1-carboxylate (960 mg, 75.1% yield) as a red solid. ¹H NMR (DMSO-d₆, 400 MHz) δ_{H}=10.45 (s, IH), 10.07 (s, 1H), 8.57 (d, J=8.8 Hz, 1H), 8.43 (d, J=2.4 Hz, 1H), 8.12 (d, J=8.8 Hz, 1H), 7.89 (dd, =24, 8.8 Hz, 1H), 7.50 (d, J=2.0 Hz, 1H), 7.36 (d, =8.8 Hz, 1H), 7.01 (d, J=2.0 Hz, 1H), 3.74 (s, 3H), 3.63-3.54 (m, 3H), 2.38-2.26 (m, 1H), 2.03-1.83 (m, 5H), 1.53-1.42 (m, 2H), 1.41-1.29 (m, 2H).

Step 5. To a solution of methyl (1r,4r)-4-((5-((6-(1-methyl-1H-pyrazol-5-yl)-3-nitropyridin-2-yl)amino)pyridin-2-yl)carbamoyl)cyclohexane-1-carboxylate (930 mg, 1.94 mmol) in DMSO (10 mL) and methanol (10 mL), 2-aminonicotinaldehyde (355 mg, 2.91 mmol) and Na₂S₂O₄ (1.69 g, 9.70 mmol) were added at 25° C. and the mixture was stirred at 100° C. for 12 hrs. The mixture was diluted with EtOAc (30 mL) and washed with brine (15 mL×3). The combined organic phase was dried over anhydrous Na₂SO₄, filtered and concentrated. The residue was subsequently dissolved in acetic acid (10 mL) and 2-aminonicotinaldehyde (237 mg, 1.94 mmol) was added into the mixture. The reaction was stirred at 100° C. for 1 hr and the suspension was subsequently degassed and purged with 02 (×3). The reaction mixture was stirred under O₂ (15 psi) at 100° C. for 1 hr. The mixture was concentrated directly. The residue was purified by Combi-Flash column (EtOAc in PE=0%-90%) to afford methyl (1r,4r)-4-((5-(2-(2-aminopyridin-3-yl)-5-(1-methyl-1H-pyrazol-5-yl)-3H-imidazo[4,5-b]pyridin-3-yl)pyridin-2-yl)carbamoyl)cyclohexane-1-carboxylate (440 mg, 41.1% yield) as a yellow solid. ¹H NMR (DMSO-d₆, 400 MHz) δ_{H}=10.68 (s, 1H), 8.43 (d, J=2.4 Hz, 1H), 8.30 (d, J=8.4 Hz, 1H), 8.23 (d, J=8.8 Hz, 1H), 8.04 (dd, J=1.6, 4.8 Hz, 1H), 7.93 (dd, J=2.4, 8.8 Hz, 1H), 7.83 (d, J=8.4 Hz, 1H), 7.47 (d, J=1.6 Hz, 1H), 7.37 (dd, J=1.6, 7.6 Hz, 1H), 6.91-6.75 (m, 3H), 6.52 (dd, J=4.8, 7.6 Hz, 1H), 4.05-4.02 (m, 3H), 3.60 (s, 3H), 2.39-2.26 (m, 2H), 1.99-1.87 (m, 4H), 1.52-1.32 (m, 4H).

Step 6. A solution of methyl (1r,4r)-4-((5-(2-(2-aminopyridin-3-yl)-5-(1-methyl-1H-pyrazol-5-yl)-3H-imidazo[4,5-b]pyridin-3-yl)pyridin-2-yl)carbamoyl)cyclohexane-1-carboxylate (100 mg, 0.180 mmol) and LiOH·H₂O (23.0 mg, 0.54 mmol) in THF (2 mL) and water (2 mL) was stirred at 50° C. for 12 hrs. The mixture was filtered directly. The mixture was purified by prep-HPLC (column: Xtimate C18 200×40 mm×7 μm; mobile phase: [water(HCl)-ACN]; B %: 0%-38%, 20.5 min) to afford (1r,4r)-4-((5-(2-(2-aminopyridin-3-yl)-5-(1-methyl-IH-pyrazol-5-yl)-3H-imidazo[4,5-b]pyridin-3-yl)pyridin-2-yl)carbamoyl)cyclohexane-1-carboxylic acid I-116 (42.8 mg, 43.2% yield) as a yellow solid. ¹H NMR (MeOD, 400 MHz) δ=8.55 (d, J=2.4 Hz, IH), 8.36 (d, J=8.4 Hz, 1H), 8.31 (d, J=9.2 Hz, 1H), 8.07-8.02 (m, 2H), 8.00 (dd, J=1.6, 7.6 Hz, 1H), 7.95 (d, J=8.4 Hz, 1H), 7.61 (d, J=2.4 Hz, 1H), 6.97-6.93 (m, 1H), 6.92 (d, J=2.4 Hz, 1H), 4.12 (s, 3H), 2.58-2.45 (m, 1H), 2.33 (tt, J=3.2, 12.0 Hz, 1H), 2.15-2.01 (m, 4H), 1.68-1.47 (m, 4H). HPLC Rₜ=3.123 min in 8 min chromatography, purity 98.3%. LCMS Rₜ=1.804 min in 4 min chromatography, purity 98.6%, MS ESI calcd. for 357.22 [M+H]⁺ 358.22, found 358.2.

### Example 108. Synthesis of (1r,4r)-4-((5-(2-(2-aminopyridin-3-yl)-6-(tert-butyl)-1H-benzo[d]imidazol-1-yl)pyridin-2-yl)carbamoyl)cyclohexane-1-carboxylic acid (Compound I-117)

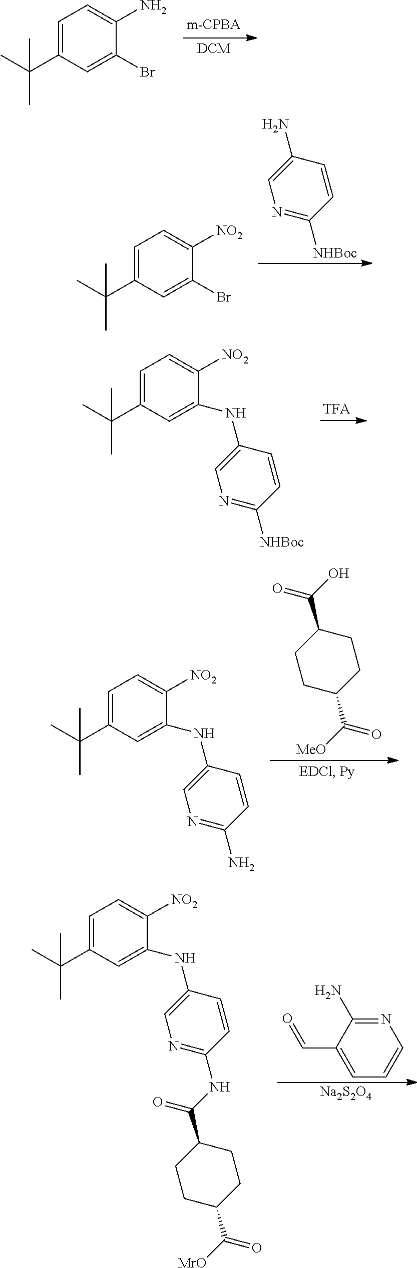

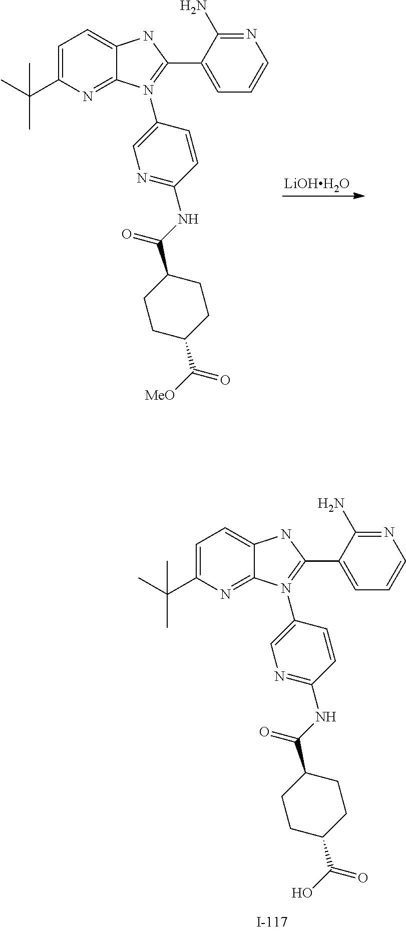

Step 1. To a solution of 2-bromo-4-(tert-butyl)aniline (9.00 g, 39.5 mmol) in DCM (300 mL), m-CPBA (30.0 g, 148 mmol) was added at 25° C. and the mixture was stirred at 70° C. for 12 hrs under N₂ atmosphere. The reaction was quenched with aqueous Na₂S₂O₃ (150 mL) and the resulting mixture was extracted with DCM (150 mL×3). The combined organic phase was washed with brine (100 mL), dried over anhydrous Na₂SO₄, filtered and concentrated. The residue was purified by Combi-Flash column (100% PE) to afford 2-bromo-4-(tert-butyl)-1-nitrobenzene (9.00 g, 88.4% yield) as a yellow oil. ¹H NMR (CDCl₃, 400 MHz) δ_{H}=7.96 (d, J 8.4 Hz, 1H), 7.85 (d, J=2.0 Hz, 1H), 7.64 (dd, J=2.0, 8.4 Hz, 1H), 1.31 (s, 9H).

Step 2. To a solution of 2-bromo-4-(tert-butyl)-1-nitrobenzene (8.60 g, 33.3 mmol) in 1,4-dioxane (100 mL), tert-butyl (5-aminopyridin-2-yl)carbamate (7.67 g, 36.7 mmol), Cs₂CO₃ (27.1 g, 83.3 mmol), Xantphos (1.45 g, 2.50 mmol) and Pd₂(dba)₃ (1.53 g, 1.67 mmol) were added at 25° C. and the mixture was stirred at 90° C. for 12 hrs. The reaction mixture was filtered and the filtrate was dissolved in water (200 mL). The resulting suspension was extracted with DCM (300 mL×3). The combined organic phase was washed with brine (100 mL), dried over anhydrous Na₂SO₄, filtered and concentrated. The residue was purified by Combi-Flash column (EtOAc in PE=0%-12%) to afford tert-butyl (5-((5-(tert-butyl)-2-nitrophenyl)amino)pyridin-2-yl)carbamate (12.2 g, 94.8% yield) as a red solid. ¹H NMR (DMSO-d₆, 400 MHz) δ_{H}=9.86 (s, 1H), 9.40 (s, 1H), 8.25 (d, J=2.4 Hz, 1H), 8.06 (d, J=8.8 Hz, 1H), 7.85 (d, J=8.8 Hz, 1H), 7.74 (dd, J=2.4, 8.8 Hz, 1H), 7.03-6.88 (m, 2H), 1.48 (s, 9H), 1.17 (s, 9H).

Step 3. A solution of tert-butyl (5-((5-(tert-butyl)-2-nitrophenyl)amino)pyridin-2-yl)carbamate (2.00 g, 5.18 mmol) in DCM (16 mL) and TFA (8.0 mL) was stirred at 25° C. for 1 hr. The mixture was concentrated directly to afford N⁵-(5-(tert-butyl)-2-nitrophenyl)pyridine-2,5-diamine (1.80 g, crude) as a yellow solid. LCMS Rₜ=0.636 min in 1.5 min chromatography, purity 89.9%, MS ESI calcd. for 286.14 [M+H]⁺ 287.14, found 287.0.

Step 4. To a solution of N5-(5-(tert-butyl)-2-nitrophenyl)pyridine-2,5-diamine (1.50 g, 5.24 mmol) in pyridine (10 mL), (1r,4r)-4-(methoxycarbonyl)cyclohexane-1-carboxylic acid (976 mg, 5.24 mmol) and EDCI (3.01 g, 15.7 mmol) were added at 25° C. and the mixture was stirred at 80° C. for 2 hrs. The mixture was diluted with EtOAc (30 mL) and washed with aqueous HCl (1 M, 10 mL×4). The combined organic phase was washed with brine (10 mL), dried over anhydrous Na₂SO₄, filtered and concentrated. The residue was purified by Combi-Flash column (EtOAc in PE=0%-25%) to afford methyl (1r,4r)-4-((5-((5-(tert-butyl)-2-nitrophenyl)amino)pyridin-2-yl)carbamoyl)cyclohexane-1-carboxylate (1.80 g, 75.6% yield) as a yellow solid. ¹H NMR (DMSO-d₆, 400 MHz) δ=10.47 (s, 1H), 9.41 (s, 1H), 8.30 (d, J=2.4 Hz, 1H), 8.14 (d, J=8.8 Hz, 1H), 8.06 (d, J=9.2 Hz, 1H), 7.77 (dd, J=2.4, 8.8 Hz, 1H), 7.02 (d, J=2.0 Hz, 1H), 6.96 (dd, J=2.0, 9.2 Hz, 1H), 3.60 (s, 3H), 2.46 (d, J=3.6 Hz, 1H), 2.33 (tt, J=3.6, 12.0 Hz, 1H), 2.02-1.83 (m, 4H), 1.54-1.42 (m, 2H), 1.41-1.29 (m, 2H), 1.18 (s, 9H).

Step 5. To a solution of methyl (1r,4r)-4-((5-((5-(tert-butyl)-2-nitrophenyl)amino)pyridin-2-yl)carbamoyl)cyclohexane-1-carboxylate (700 mg, 1.54 mmol) in DMSO (6 mL) and methanol (6 mL), 2-aminonicotinaldehyde (188 mg, 1.54 mmol) and Na₂S₂O₄ (1.61 g, 9.24 mmol) were added at 25° C. and the mixture was stirred at 100° C. for 12 hrs. The mixture was diluted with EtOAc (20 mL) and washed with brine (10 mL×3). The combined organic phase was dried over anhydrous Na₂SO₄, filtered and concentrated. The residue was purified by Combi-Flash column (EtOAc in PE=0%-50%) to afford methyl (1r,4r)-4-((5-(2-(2-aminopyridin-3-yl)-6-(tert-butyl)-1 H-benzo[d]imidazol-1-yl)pyridin-2-yl)carbamoyl)cyclohexane-1-carboxylate (762 mg, 94.0% yield) as a yellow solid. ¹H NMR (DMSO-d₆, 400 MHz) δ=10.68 (s, 1H), 8.35-8.21 (m, 2H), 8.00-7.89 (m, 2H), 7.72 (d, J=8.4 Hz, 1H), 7.43 (dd, J=1.6, 8.8 Hz, 1H), 7.20 (dd, J=1.6, 7.6 Hz, 1H), 7.12 (d, J=1.2 Hz, 1H), 6.82 (s, 2H), 6.44 (dd, J=4.8, 7.6 Hz, 1H), 3.60 (s, 3H), 2.40-2.26 (m, 1H), 2.01-1.86 (m, 5H), 1.54-1.42 (m, 2H), 1.42-1.33 (m, 2H), 1.30 (s, 9H).

Step 6. A solution of methyl (1r,4r)-4-((5-(2-(2-aminopyridin-3-yl)-6-(tert-butyl)-1H-benzo[d]imidazol-1-yl)pyridin-2-yl)carbamoyl)cyclohexane-1-carboxylate (150 mg, 0.290 mmol) and LiOH·H₂O (36.0 mg, 0.850 mmol) in THF (1 mL) and water (1 mL) was stirred at 25° C. for 1 hr. The mixture was filtered directly. The mixture was purified by prep-HPLC (column: Xtimate C18 200*40 mm*7 μm; mobile phase: [water(HCl)-ACN], B %: 10%-50%, 20.5 min) to afford (1r,4r)-4-((5-(2-(2-aminopyridin-3-yl)-6-(tert-butyl)-1H-benzo[d]imidazol-1-yl)pyridin-2-yl)carbamoyl)cyclohexane-1-carboxylic acid I-117 (127.7 mg, 86.0% yield) as a yellow solid. ¹H NMR (MeOD, 400 MHz) δ=8.54 (d, J=2.4 Hz, 1H), 8.31 (d, J=9.2 Hz, 1H), 8.26 (dd, J=1.6, 7.6 Hz, 1H), 8.16 (dd, J=1.6, 6.4 Hz, 1H), 8.11 (dd, J=2.8, 9.2 Hz, 1H), 7.96-7.91 (m, 1H), 7.84 (dd, J=1.6, 8.8 Hz, 1H), 7.46 (d, J=1.2 Hz, 1H), 7.08 (dd, J=6.4, 7.6 Hz, 1H), 2.59-2.47 (m, 1H), 2.33 (tt, =3.2, 12.0 Hz, 1H), 2.17-1.97 (m, 4H), 1.67-1.47 (m, 4H), 1.39 (s, 9H). HPLC Rₜ=2.804 min in 8 min chromatography, purity 98.3%. LCMS Rₜ=1.868 min in 4 min chromatography, purity 98.5%, MS ESI calcd. for 512.25 [M+H]⁺ 513.25. found 513.3.

### Example 109. Synthesis of (1r,4r)-4-((5-(2-(2-aminopyridin-3-yl)-5-phenyl-3H-imidazo[4,5-b]pyridin-3-yl)-6-methylpyridin-2-yl)carbamoyl)cyclohexane-1-carboxylic acid (Compound I-118)

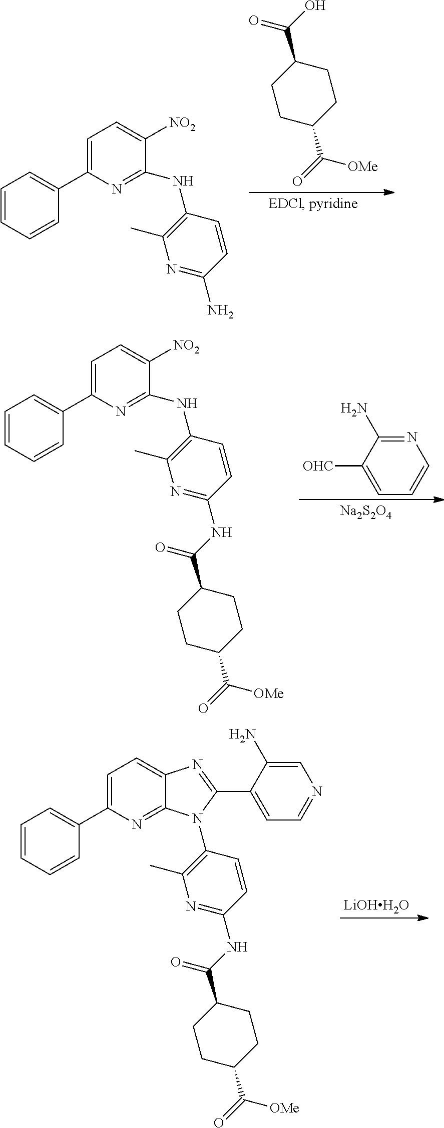

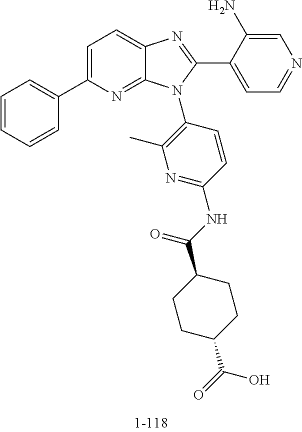 [SMILES: Cc1nc(NC(=O)[C@H]2CC[C@H](C(=O)O)CC2)ccc1-n1c(-c2ccncc2N)nc2ccc(-c3ccccc3)nc21]

Step 1. To a stirred solution of 6-methyl-N⁵-(3-nitro-6-phenyl-2-pyridyl)pyridine-2,5-diamine (1.00 g, 3.11 mmol) and (1r,4r)-4-(methoxycarbonyl)cyclohexane-1-carboxylic acid (579 mg, 3.11 mmol) in pyridine (15 mL), EDCI (657 mg, 3.42 mmol) was added and the reaction was stirred at 80° C. under N₂ for 12 hrs. The reaction mixture was diluted with EtOAc (50 mL) and washed with water (50 mL) and brine (50 mL×2), dried over anhydrous sodium sulfate, filtered and the filtrate was concentrated. The crude material was purified by flash chromatography on silica gel (25-30% of EtOAc in PE) to afford methyl (1r,4r)-4-((6-methyl-5-((3-nitro-6-phenylpyridin-2-yl)amino)pyridin-2-yl)carbamoyl) cyclohexane-1-carboxylate (500 mg, 32.8% yield) as a brown solid. ¹H NMR (400 MHz, DMSO-d₆) δ=10.44 (s, 1H), 9.94 (s, 1H), 8.61 (d, J=8.8 Hz, 1H), 8.13-7.87 (m, 4H), 7.66-7.38 (m, 4H), 3.60 (s, 3H), 2.38 (s, 3H), 2.35-2.29 (m, 1H), 1.98-1.83 (m, 4H), 1.55-1.28 (m, 5H).

Step 2. To a stirred solution of 2-aminopyridine-3-carbaldehyde (100 mg, 0.817 mmol) and methyl (1r,4r)-4-((6-methyl-5-((3-nitro-6-phenylpyridin-2-yl)amino)pyridin-2-yl)carbamoyl) cyclohexane-1-carboxylate (200 mg, 0.41 mmol) in DMSO (5 mL) and methanol (1 mL), Na₂S₂O₄ (284 mg, 1.63 mmol) was added and the reaction mixture was stirred at 100° C. under O₂ for 12 hrs. The reaction mixture was diluted with EtOAc (15 mL), washed with water (20 mL) and brine (20 mL×2), dried over anhydrous sodium sulfate, filtered and the filtrate was concentrated. The crude material was purified by flash chromatography on silica gel (95% EtOAc in PE) to afford methyl (1r,4r)-4-((5-(2-(2-aminopyridin-3-yl)-5-phenyl-3H-imidazo[4,5-b]pyridin-3-yl)-6-methylpyridin-2-yl)carbamoyl)cyclohexane-1-carboxylate (80.0 mg, 34.9% yield) as a yellow solid. ¹H NMR (400 MHz, DMSO-d₆) δ=10.68 (s, 1H), 8.30 (d, J=8.4 Hz, 1H), 8.11 (d, J=8.8 Hz, 1H), 8.05-7.95 (m, 4H), 7.87 (d, J=8.4 Hz, 1H), 7.53-7.30 (m, 3H), 7.24-7.09 (m, 3H), 6.46 (dd, J=4.8, 8.0 Hz, 1H), 3.60 (s, 3H), 3.40-3.34 (m, 1H), 2.36-2.28 (m, 1H), 1.98-1.80 (m, 4H), 1.58-1.29 (m, 4H).

Step 3. To a stirred solution of trans-methyl 4-[[5-[2-(2-amino-3-pyridyl)-5-phenyl-imidazo[4,5-b]pyridin-3-yl]-6-methyl-2-pyridyl]carbamoyl]cyclohexanecarboxylate (80.0 mg, 0.14 mmol) in THF (1 mL) and water (1 mL), LiOH·H₂O (17.0 mg, 0.43 mmol) was added and the reaction mixture was stirred at 30° C. under N₂ for 2 hrs. The reaction mixture was concentrated and adjusted to pH ~5 with 1N HCl. The crude product was purified by prep-HPLC (column: Welch Xtimate C18 150×30 mm×5 μm, method: water (FA)-ACN, begin B: 25, end B: 55) to afford (1r,4r)-4-((5-(2-(2-aminopyridin-3-yl)-5-phenyl-3H-imidazo[4,5-b]pyridin-3-yl)-6-methylpyridin-2-yl)carbamoyl)cyclohexane-1-carboxylic acid I-118 (63 mg, 80.1% yield) as a white solid ¹H NMR (400 MHz, DMSO-d₆) δ=12.10 (s, 1H), 10.68 (s, 1H), 8.30 (d, J=8.4 Hz, 1H), 8.15-8.09 (m, 1H), 8.06-7.97 (m, 4H), 7.87 (d, J=8.4 Hz, 1H), 7.53-7.33 (m, 3H), 7.24-7.12 (m, 3H), 6.47 (dd, J=4.8, 7.6 Hz, 1H), 2.56-2.52 (m, 1H), 2.21 (s, 1H), 2.07 (s, 3H), 2.01-1.84 (m, 4H), 1.56-1.19 (m, 4H). HPLC Rₜ=3.106 min in 8 min chromatography, purity 100%. LCMS Rₜ=2.515 min in 4 min chromatography, purity 100%, MS ESI calcd. for 547.23 [M+H]⁺ 548.23, found 548.1.

### Example 110. Synthesis of (1r,4r)-4-((5-(2-(2-aminopyridin-3-yl)-5-cyclohexyl-3H-imidazo[4,5-b]pyridin-3-yl)pyridin-2-yl)carbamoyl)cyclohexane-1-carboxylic acid (Compound I-119)

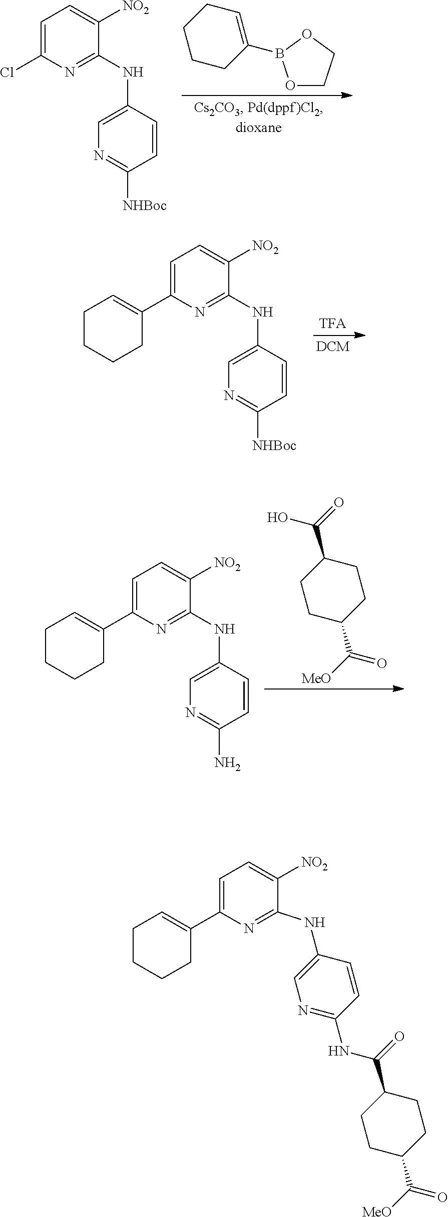 [SMILES: CC(C)(C)OC(=O)Nc1ccc(Nc2nc(Cl)ccc2[N+](=O)[O-])cn1 ; C1=C(B2OCCO2)CCCC1 ; CC(C)(C)OC(=O)Nc1ccc(Nc2nc(C3=CCCCC3)ccc2[N+](=O)[O-])cn1 ; Nc1ccc(Nc2nc(C3=CCCCC3)ccc2[N+](=O)[O-])cn1 ; COC(=O)[C@H]1CC[C@H](C(=O)O)CC1 ; COC(=O)[C@H]1CC[C@H](C(=O)Nc2ccc(Nc3nc(C4=CCCCC4)ccc3[N+](=O)[O-])cn2)CC1]

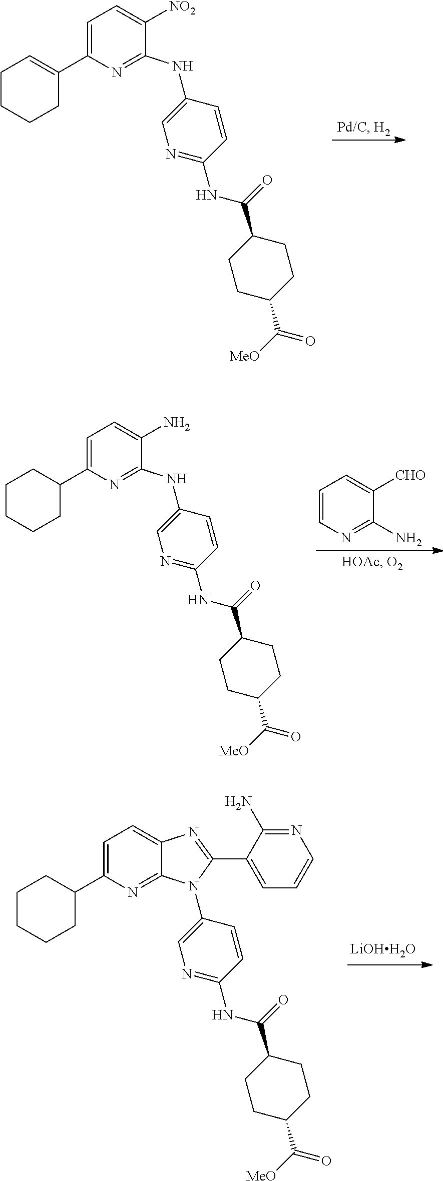 [SMILES: COC(=O)[C@H]1CC[C@H](C(=O)Nc2ccc(Nc3nc(C4=CCCCC4)ccc3[N+](=O)[O-])cn2)CC1 ; COC(=O)[C@H]1CC[C@H](C(=O)Nc2ccc(Nc3nc(C4CCCCC4)ccc3N)cn2)CC1 ; Nc1ncccc1C=O ; COC(=O)[C@H]1CC[C@H](C(=O)Nc2ccc(-n3c(-c4cccnc4N)nc4ccc(C5CCCCC5)nc43)cn2)CC1 ; "LiOH·H2O"]

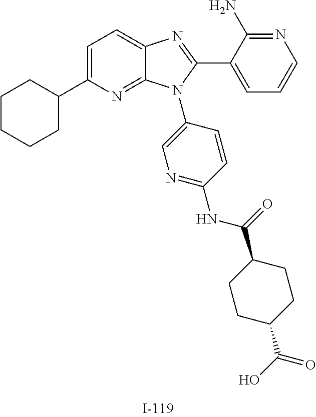 [SMILES: Nc1ncccc1-c1nc2ccc(C3CCCCC3)nc2n1-c1ccc(NC(=O)[C@H]2CC[C@H](C(=O)O)CC2)nc1]

Step 1. To a solution of tert-butyl (5-((6-chloro-3-nitropyridin-2-yl)amino)pyridin-2-yl)carbamate (1.50 g, 4.10 mmol) and 2-(cyclohexen-1-yl)-4,4,5,5-tetramethyl-1,3,2-dioxaborolane (1.02 g, 4.92 mmol) in 1,4-dioxane (12.0 mL), Cs₂CO₃ (4.01 g, 12.3 mmol) and Pd(dppf)Cl₂ (300 mg, 0.410 mmol) were added and the mixture was stirred at 80° C. for 2 hrs. The reaction mixture was concentrated. Water (50 mL) was added and the resulting mixture was extracted with EtOAc (40 mL×3). The combined organic phase was washed with brine (50 mL), dried over anhydrous Na₂SO₄, filtered and concentrated. The residue was purified by flash silica gel chromatography (eluent of 0%-15% MeOH/DCM) to give tert-butyl (5-((6-(cyclohex-1-en-1-yl)-3-nitropyridin-2-yl)amino)pyridin-2-yl)carbamate (803 mg, 47.6% yield) as a yellow solid. ¹H NMR (400 MHz, DMSO-d₆) δ=10.02 (s, 1H), 9.68 (s, 1H), 8.54 (d, J=2.0 Hz, 1H), 8.44 (d, J=8.8 Hz, 1H), 7.99 (dd, J=2.4, 8.8 Hz, 1H), 7.80 (d, J=8.8 Hz, 1H), 7.14 (d, J=8.4 Hz, 1H), 6.91 (s, 1H), 2.40-2.36 (m, 2H), 2.28-2.22 (m, 2H), 1.73-1.65 (m, 2H), 1.63-1.56 (m, 2H), 1.48 (s, 9H).

Step 2. To a solution of tert-butyl (5-((6-(cyclohex-1-en-1-yl)-3-nitropyridin-2-yl)amino)pyridin-2-yl)carbamate (800 mg, 1.94 mmol) in DCM (8 mL), TFA (2.00 mL) was added and the mixture was stirred at 25° C. for 3 hr. The reaction mixture was concentrated directly to give N⁵-(6-(cyclohex-1-en-1-yl)-3-nitropyridin-2-yl)pyridine-2,5-diamine (800 mg, crude) as a brown solid. ¹H NMR (400 MHz, DMSO-d₆) δ=9.97 (s, 1H), 8.47 (d, J=8.8 Hz, 1H), 8.29 (d, J=2.4 Hz, 1H), 8.16 (dd, J=2.4, 9.6 Hz, 1H), 8.10-7.82 (m, 2H), 7.22 (d, J=8.8 Hz, 1H), 7.01 (d, J=9.6 Hz, 1H), 6.96-6.90 (m, 1H), 2.39-2.32 (m, 1H), 2.29-2.22 (m, 1H), 1.72-1.65 (m, 2H), 1.63-1.56 (m, 2H).

Step 3. To a solution of N⁵-(6-(cyclohex-1-en-1-yl)-3-nitropyridin-2-yl)pyridine-2,5-diamine (800 mg, 2.09 mmol) and trans-4-methoxycarbonylcyclohexanecarboxylic acid (583 mg, 3.13 mmol) in DCM (10 mL), DIEA (808 mg, 6.26 mmol) and HATU (1.19 mg, 3.13 mmol) were added and the mixture was stirred at 100° C. for 12 hrs. The reaction mixture was concentrated directly. Water (20 mL) was added and the resulting mixture was extracted with EtOAc (15 mL×3). The combined organic phase was washed with brine (15 mL), dried over anhydrous Na₂SO₄, filtered and concentrated. The residue was purified by flash silica gel chromatography (eluent of 0%-7% MeOH/DCM) to give methyl (1r,4r)-4-((5-((6-(cyclohex-1-en-1-yl)-3-nitropyridin-2-yl)amino)pyridin-2-yl)carbamoyl)cyclohexane-1-carboxylate (1.2 g, 76.4% yield) as a yellow solid. ¹H NMR (400 MHz, DMSO-d₆) δ=10.18 (s, 1H), 8.88 (s, 1H), 8.58 (d, J=2.4 Hz, 1H), 8.45 (d, J=8.8 Hz, 1H), 8.31 (d, J=8.8 Hz, 1H), 8.10 (dd, J=2.4, 8.8, 1H), 7.00 (d, J=8.8 Hz, 1H), 6.94-6.89 (s, 1H), 2.80 (s, 3H), 2.47-2.45 (m, 1H), 2.37-2.34 (m, 1H), 2.33-2.29 (m, 4H), 2.11-2.10 (m, 2H), 1.79-1.75 (m, 2H), 1.68-1.65 (m, 2H), 1.63-1.60 (m, 1H), 1.59-1.56 (m, 1H), 1.55-1.53 (m, 1H), 1.52-1.46 (m, 3H).

Step 4. To a solution of methyl (1r,4r)-4-((5-((6-(cyclohex-1-en-1-yl)-3-nitropyridin-2-yl)amino)pyridin-2-yl)carbamoyl)cyclohexane-1-carboxylate (1.10 g, 2.29 mmol) in methanol (5.00 mL) and THF (5 mL), wet Pd/C (0.75 g, 10% purity) was added under Ar. The reaction mixture was degassed and purged with H₂ (×3) and the mixture was stirred at 25° C. for 12 hrs under H₂ (15 psi). The mixture was filtered through celite and washed with MeOH in DCM (20%, 30 mL×4). The filtrate was concentrated to give methyl (1r,4r)-4-((5-((3-amino-6-cyclohexylpyridin-2-yl)amino)pyridin-2-yl)carbamoyl)cyclohexane-1-carboxylate (890 mg, 85.9% yield) as a yellow solid. ¹H NMR (400 MHz, DMSO-d₆) δ=8.99 (s, 1H), 8.48 (s, 1H), 8.19 (d, J=8.8 Hz, 1H), 7.90 (dd, J=1.6, 9.2 Hz, 1H), 6.98 (d, J=7.6 Hz, 1H), 6.60 (d, J=7.6 Hz, 1H), 4.68-4.62 (m, 2H), 3.48 (d, J=1.2 Hz, 3H), 2.52 (t, J=11.2 Hz, 2H), 2.35-2.29 (m, 41), 2.08 (s, 2H), 1.90 (d, J=12.0 Hz, 21), 1.82 (d, J=12.4 Hz, 2H), 1.74-1.70 (m, 1H), 1.63-1.56 (m, 2H), 1.49-1.47 (m, 2H), 1.43-1.35 (m, 2H), 1.29-1.22 (m, 2H).

Step 5. To a solution of methyl (1r,4r)-4-((5-((3-amino-6-cyclohexylpyridin-2-yl)amino)pyridin-2-yl)carbamoyl)cyclohexane-1-carboxylate (500 mg, 1.11 mmol) in acetic acid (5 mL), 2-aminopyridine-3-carbaldehyde (203 mg, 1.66 mmol) was added under N₂. The reaction mixture was degassed and purged with 02 (×3) and the mixture was stirred at 80° C. for 2 hrs. The reaction mixture was concentrated. The residue was purified by flash silica gel chromatography (eluent of 0%-9% MeOH/DCM) to give methyl (1r,4r)-4-((5-(2-(2-aminopyridin-3-yl)-5-cyclohexyl-3H-imidazo[4,5-b]pyridin-3-yl)pyridin-2-yl)carbamoyl)cyclohexane-1-carboxylate (434 mg, 56.6% yield) as a brown solid. ¹H NMR (400 MHz, DMSO-d₆) δ=9.87 (s, 1H), 8.46 (d, J=8.8 Hz, 1H), 8.29 (d, J=2.4 Hz, 1H), 8.25 (dd, J=2.0, 4.8 Hz, 1H), 8.19 (s, 1H), 8.02 (d, J=8.4 Hz, 1H), 7.93 (dd, J=1.6, 5.6 Hz, 1H), 7.83 (dd, J=2.0, 7.6 Hz, 1H), 7.30 (dd, J=1.2, 7.6 Hz, 1H), 7.24 (d, J=8.4 Hz, 1-i), 6.52 (dd, J 5.6, 7.6 Hz, 1H), 3.70 (s, 3H), 2.79-2.71 (m, 1H), 2.38-2.32 (m, 2H), 1.94-1.80 (m, 6H), 1.77-1.62 (m, 4H), 1.59 (dd, J=1.6, 8.8 Hz, 1H), 1.56-1.48 (m, 4H), 1.47-1.35 (m, 3H).

Step 6. To a solution of methyl (1r,4r)-4-((5-(2-(2-aminopyridin-3-yl)-5-cyclohexyl-3H-imidazo[4,5-b]pyridin-3-yl)pyridin-2-yl)carbamoyl)cyclohexane-1-carboxylate (220 mg, 0.397 mmol) in THF (2 mL), water (2 mL) and LiOH·H₂O (83.0 mg, 1.99 mmol) were added and the mixture was stirred at 25° C. for 12 hrs. The reaction mixture was concentrated directly. The residue was purified by prep-HPLC (Column Welch Xtimate C18 40*200 mm 7 μm; mobile phase: [water (NH₄HCO₃)-ACN]; B %. 8%-48%; 25 min) to give (1r,4r)-4-((5-(2-(2-aminopyridin-3-yl)-5-cyclohexyl-3H-imidazo[4,5-b]pyridin-3-yl)pyridin-2-yl)carbamoyl)cyclohexane-1-carboxylic acid I-119 (36.4 mg, 16.9% yield) as a white solid. ¹H NMR (400 MHz, DMSO-d₆) δ=10.62 (s, 1H), 8.34 (d, J=2.4 Hz, 1H), 8.20 (d, J=8.8 Hz, IH), 8.11 (d, J=8.4 Hz, 1H), 8.01 (dd, J=1.6, 4.8 Hz, 1H), 7.85 (dd, J=2.4, 8.8 Hz, 1H), 7.33-7.28 (m, 2H), 6.73 (s, 2H), 6.49 (dd, J=4.8, 7.6 Hz, 1H), 2.73 (tt, J=3.2, 11.6 Hz, 1H), 2.21 (tt, J=3.2, 12.0 Hz, 1H), 1.99-1.76 (m, 8H), 1.69 (d, J=12.0 Hz, 1H), 1.55-1.18 (m, 10H). HPLC Rₜ=2 832 min in 8.0 min chromatography, purity 99.7%. LCMS Rₜ=1.312 min in 2 min chromatography, purity 97.0%, MS ESI calcd. for 539.26 [M+H]⁺ 540.26, found 540.2.

### Example 111. Synthesis of (1r,4r)-4-((5-(2-(2-aminopyridin-3-yl)-5-cyclopentyl-3H-imidazo[4,5-b]pyridin-3-yl)pyridin-2-yl)carbamoyl) cyclohexane-1-carboxylic acid (Compound I-120)

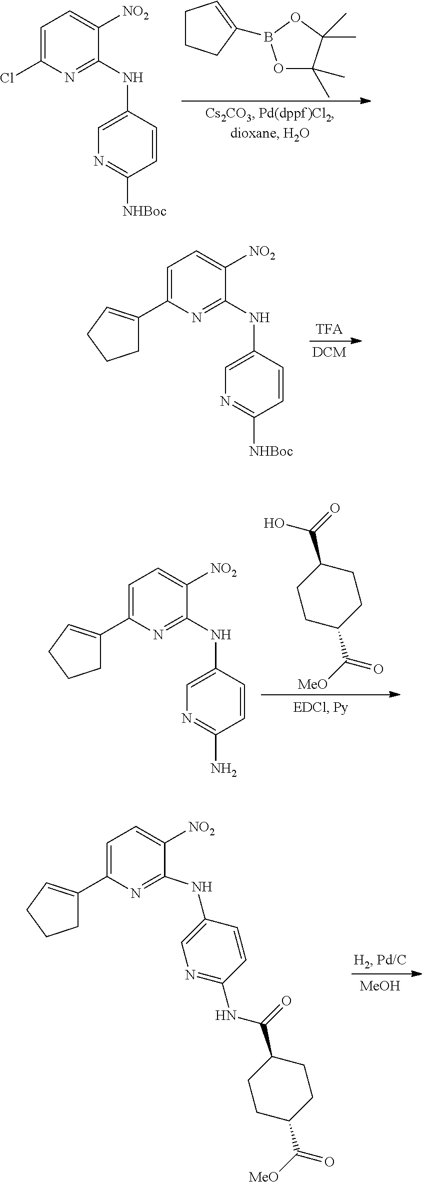

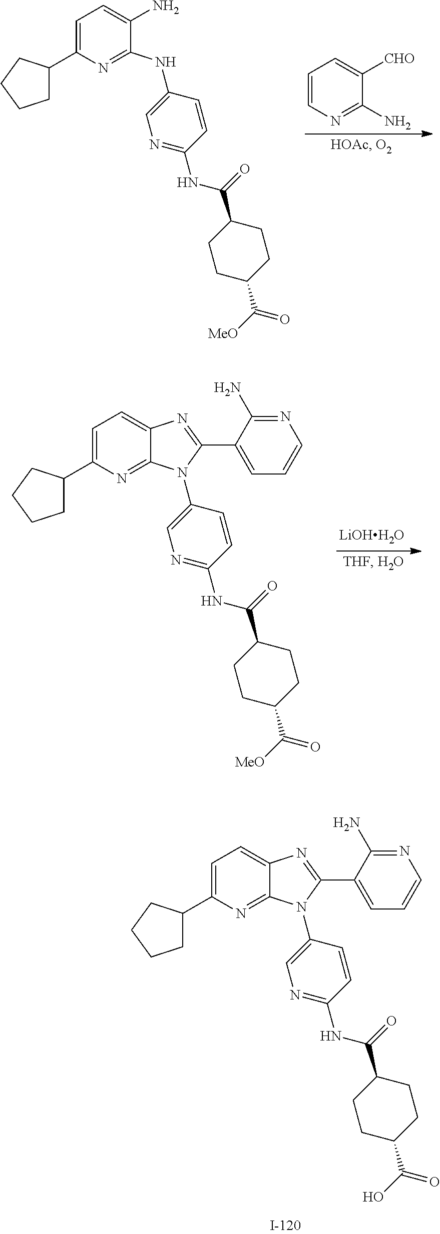 [SMILES: COC(=O)[C@H]1CC[C@H](C(=O)Nc2ccc(Nc3nc(C4CCCC4)ccc3N)cn2)CC1 ; Nc1ncccc1C=O ; COC(=O)[C@H]1CC[C@H](C(=O)Nc2ccc(-n3c(-c4cccnc4N)nc4ccc(C5CCCC5)nc43)cn2)CC1 ; "LiOH·H2O" ; Nc1ncccc1-c1nc2ccc(C3CCCC3)nc2n1-c1ccc(NC(=O)[C@H]2CC[C@H](C(=O)O)CC2)nc1]

Step 1. A mixture of tert-butyl (5-((6-chloro-3-nitropyridin-2-yl)amino)pyridin-2-yl)carbamate (1.50 g, 4.10 mmol), 2-(cyclopenten-1-yl)-4,4,5,5-tetramethyl-1,3,2-dioxaborolane (955 mg, 4.92 mmol), Cs₂CO₃ (4.01 g, 12.3 mmol), water (5 mL) in 1,4-Dioxane (20 mL) was degassed and purged with N₂ (3). Pd(dppf)Cl₂ (300 mg, 0.410 mmol) was then added to the mixture and the mixture was stirred at 80° C. for 12 hrs under N₂ atmosphere. Water (2 mL) was added to the reaction mixture and the reaction mixture was filtered. PE (10 m L) and EtOAc(1 mL) were then added to the filter cake and the mixture was stirred at 25° C. for 12 hrs. The reaction mixture was filtered and the filter cake was concentrated to give tert-butyl (5-((6-(cyclopent-1-en-1-yl)-3-nitropyridin-2-yl)amino)pyridin-2-yl)carbamate (1.18 g, 72.4% yield) as a brown solid, which was used in the next step without further purification. ¹H NMR (400 MHz, DMSO-d₆) δ=10.18 (s, 1H), 8.57 (d, J=2.4 Hz, 1H), 8.47 (d, J=8.4 Hz, 1H), 8.18 (dd, J=2 8, 9.2 Hz, 1H), 8.01 (d, J=9.2 Hz, 1H), 7.51 (s, 1H), 6.98 (d, J=8.8 Hz, 1H), 6.81 (t, J=1.6 Hz, 1H), 2.83-2.75 (m, 2H), 2.67-2.60 (in. 2H), 2.07 (quin, J=7.6 Hz, 2H), 1.57 (s, 9H).

Step 2. To a solution of tert-butyl (5-((6-(cyclopent-1-en-1-yl)-3-nitropyridin-2-yl)amino)pyridin-2-yl)carbamate (1.18 g, 2.97 mmol) in DCM (15 mL), TFA (3.39 g, 29.7 mmol) was added and the mixture was stirred at 25° C. for 2 hrs. The reaction mixture was concentrated directly to give N⁵-(6-(cyclopent-1-en-1-yl)-3-nitropyridin-2-yl)pyridine-2,5-diamine (880 mg, 99.7% yield) as a brown oil, which was used in the next step without further purification. LCMS Rₜ=0.508 min in 1.0 min chromatography, purity 96.2%, MS ESI calcd. for 297.12 [M+H]⁺ 298.12, found 298.1.

Step 3. To a solution of N⁵-(6-(cyclopent-1-en-1-yl)-3-nitropyridin-2-yl)pyridine-2,5-diamine (1.00 g, 3.36 mmol) and (1r,4r)-4-(methoxycarbonyl)cyclohexane-1-carboxylic acid (1.25 g, 6.73 mmol) in pyridine (3 mL), EDCI (1.29 g, 6.73 mmol) was added under N₂ and the mixture was stirred at 80° C. for 1 hr. The reaction mixture was diluted with EtOAc (50 mL) and washed with brine (10 mL×3). The combined organic phase was dried over anhydrous Na₂SO₄, filtered and concentrated. The residue was purified by flash silica gel chromatography (eluent of 0-5% MeOH in DCM) to give methyl (1r,4r)-4-((5-((6-(cyclopent-1-en-1-yl)-3-nitropyridin-2-yl)amino)pyridin-2-yl)carbamoyl)cyclohexane-1-carboxylate (880 mg, 24.2% yield) as a red solid. ¹H NMR (400 MHz, DMSO-d₆) δ=10.45 (s, 1H), 10.11 (s, 1H), 8.70 (br s, 1H), 8.51 (d, J 8.4 Hz, 1H), 8.16-8.08 (m, 2H), 7.26 (d, J=8.8 Hz, 1H), 6.90 (s, 1H), 3.64 (s, 3H), 2.71 (d, J=1.6 Hz, 2H), 2.32 (dd, J=3.2, 8.4 Hz, 3H), 2.24-2.17 (m, 3H), 1.98 (d, J=8.0 Hz, 4H), 1.40 (d, J=5.2 Hz, 4H).

Step 4. To a solution of methyl (1r,4r)-4-((5-((6-(cyclopent-1-en-1-yl)-3-nitropyridin-2-yl)amino)pyridin-2-yl)carbamoyl)cyclohexane-1-carboxylate (880 mg, 1.89 mmol) in methanol (10 mL), Pd/C (10%, 0.3 g) was added under N₂ atmosphere. The suspension was degassed and purged with H₂ (×3) and the reaction mixture was stirred under H₂ (50 psi) at 80° C. for 12 hrs. The reaction mixture was filtered and the filtrate was concentrated to give methyl (1r,4r)-4-((5-((3-amino-6-cyclopentylpyridin-2-yl)amino)pyridin-2-yl)carbamoyl)cyclohexane-1-carboxylate (1.00 g, crude) as a brown oil, which was used in the next step without further purification. LCMS Rₜ=0.772 min in 1.5 min chromatography, purity 78.4%, MS ESI calcd for 437.24 [M+H]+ 438.24, found 483.2.

Step 5. A solution of methyl (1r,4r)-4-((5-((3-amino-6-cyclopentylpyridin-2-yl)amino)pyridin-2-yl)carbamoyl)cyclohexane-1-carboxylate (500 mg, 1.14 mmol) and 2-aminopyridine-3-carbaldehyde (279 mg, 2.29 mmol) in acetic acid (5 mL) under O₂ was stirred at 100° C. for 12 hrs. The reaction mixture was diluted with EtOAc (30 mL) and washed with sat. aq. NaHCO₃ (10 mL×3). The combined organic phase was washed with brine (5 mL), dried over anhydrous Na₂SO₄, filtered and concentrated. The residue was purified by flash silica gel chromatography (eluent of 0-6% MeOH in DCM) to give methyl (1r,4r)-4-((5-(2-(2-aminopyridin-3-yl)-5-cyclopentyl-3H-imidazo[4,5-b]pyridin-3-yl)pyridin-2-yl)carbamoyl) cyclohexane-1-carboxylate (100 mg, 16.3% yield) as a yellow oil ¹H NMR (400 MHz, DMSO-d₆) δ=10.62 (s, 1H), 8.34 (d, J=2.4 Hz, 1H), 8.19 (d, J=8.8 Hz, 1H), 8.09 (d, J=8.4 Hz, 1H), 8.00 (dd, J=1.6, 4.8 Hz, 1H), 7.84 (dd, J=2.4, 8.8 Hz, 1H), 7.35-7.23 (m, 2H), 6.73 (d, J=4.0 Hz, 1H), 6.49 (dd, J=4.8, 7.6 Hz, 1H), 3.60 (s, 3H), 2.52 (s, 2H), 2.35-2.29 (m, 1H), 1.99-1.88 (m, 6H), 1.77-1.64 (m, 5H), 1.52-1.29 (m, 5H).

Step 6. To a solution of methyl (1r,4r)-4-((5-(2-(2-aminopyridin-3-yl)-5-cyclopentyl-3H-imidazo[4,5-b]pyridin-3-yl)pyridin-2-yl)carbamoyl) cyclohexane-1-carboxylate (100 mg, 0.185 mmol) in THF (1 mL), LiOH·H₂O (23 mg, 0.556 mmol) and water (0.5 mL) were added and the mixture was stirred at 25° C. for 2 hrs. The reaction mixture was filtered, and the filtrate was concentrated to dryness. The residue was purified by prep-HPLC (Column: Welch Xtimate C18 40*200 mm 7 μm; mobile phase. [water (NH₄HCO₃)-ACN]; B %: 4%-44%, 25 min) to give (1r,4r)-4-((5-(2-(2-aminopyridin-3-yl)-5-cyclopentyl-3H-imidazo[4,5-b]pyridin-3-yl)pyridin-2-yl)carbamoyl) cyclohexane-1-carboxylic acid 1-120 (45.0 mg, 45.8% yield) as a white solid. ¹H NMR (400 MHz, DMSO-d₆) δ=10.62 (s, 1H), 8.35 (d, J=2.8 Hz, 1H), 8.19 (d, J=8.8 Hz, IH), 8.09 (d, J=8.0 Hz, 1H), 8.00 (dd, J=1.6, 4.8 Hz, 1H), 7.84 (dd, J=2.8, 8.8 Hz, 1H), 7.34-7.27 (m, 2H), 6.74 (s, 2H), 6.49 (dd, J=4.8, 7.6 Hz, 1H), 3.26-3.20 (m, 2H), 2.20 (tt, J=3.6, 12.0 Hz, 1H), 2.00-1.86 (m, 6H), 1.77-1.59 (m, 6H), 1.52-1.41 (m, 2H), 1.39-1.27 (m, 2H). HPLC Rₜ=3.110 min in 8 min chromatography, purity 99.8%. LCMS Rₜ=1.252 min in 2.0 min chromatography, purity 100.%, MS ESI calcd. for 525.25 [M+H]⁺ 526.25, found 526.2.

### Example 112. Synthesis of (1r,4r)-4-((5-(2-(2-aminopyridin-3-yl)-5-phenyl-3H-imidazo[4,5-b]pyridin-3-yl)-6-methylpyridin-2-yl)amino)cyclohexane-1-carboxylic acid (Compound I-121)

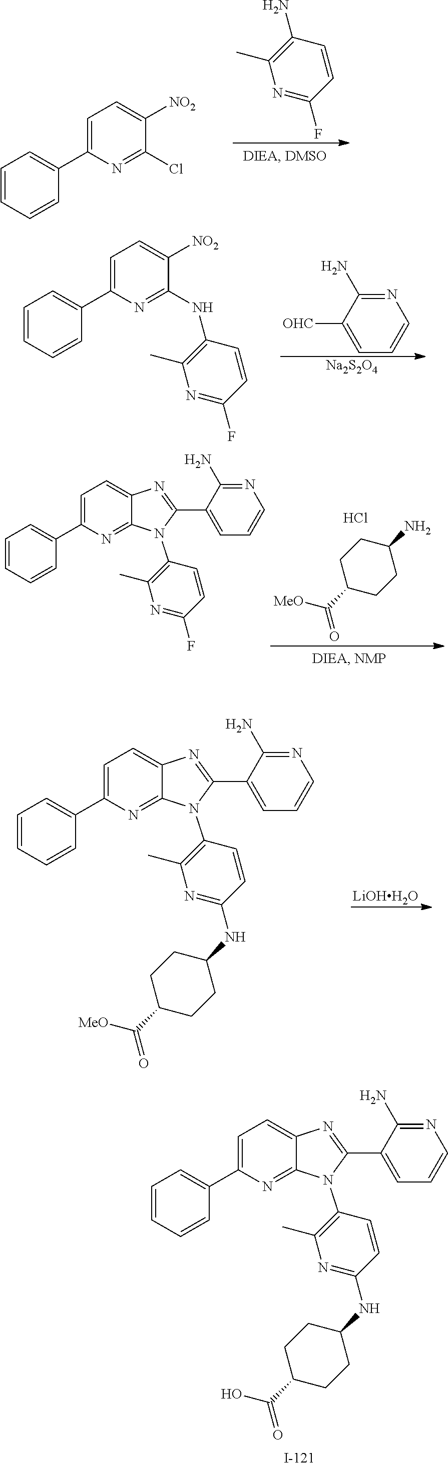

Step 1. To a solution of 2-chloro-3-nitro-6-phenyl-pyridine (2.00 g, 8.52 mmol) in DMSO (15 mL), 6-fluoro-2-methyl-pyridin-3-amine (2.15 g, 17.0 mmol) was added and the reaction stirred at 100° C. for 16 hrs. Water (150 mL) was added to the reaction mixture and the whole extracted with EtOAc (100 mL×3). The organic layer was dried over Na₂SO₄ and concentrated in vacuum to provide the crude material. The crude was purified by flash column (0-40% of EtOAc in PE) to give 6-fluoro-2-methyl-N-(3-nitro-6-phenyl-2-pyridyl)pyridin-3-amine (2.70 g, 97.7% yield) as a yellow solid. ¹H NMR (400 MHz, DMSO-d₆) δ=9.94 (s, 1H), 8.64-8.60 (m, 1H), 8.28 (d, J=8.4 Hz, 1H), 8.18-8.12 (m, 2H), 7.94-7.86 (m, 2H), 7.61-7.54 (m, 3H), 7.51-7.44 (m, 3H), 7.16-7.04 (m, 1H), 2.38 (s, 3H). ¹⁹F NMR (376 MHz, DMSO-d₆) δ=−72.182.

Step 2. To a solution of 6-fluoro-2-methyl-N-(3-nitro-6-phenyl-2-pyridyl)pyridin-3-amine (2.20 g, 6.85 mmol) in DMSO (22 mL) and methanol (3 mL), Na₂S₂O₄ (4.77 g, 27.4 mmol) and 2-aminopyridine-3-carbaldehyde (1.17 g, 9.58 mmol) were added and the reaction stirred at 100° C. for 16 hrs. Water (300 mL) was added to the reaction mixture and the whole extracted with EtOAc (200 mL×3). The organic layer was washed with brine then dried over Na₂SO₄ and concentrated in vacuum to give a crude. The crude was purified by flash column (0-80% of EtOAc in PE) to give 3-(3-(6-fluoro-2-methylpyridin-3-yl)-5-phenyl-3H-imidazo[4,5-b]pyridin-2-yl)pyridin-2-amine (600 mg, 22.3% yield) as a yellow oil. ¹H NMR (400 MHz, DMSO-d₆) δ=8.35-8.28 (m, 1H), 8.15-8.09 (m, 1H), 8.05-7.99 (m, 4H), 7.49-7.45 (m, 2H), 7.43-7.38 (m, 1H), 7.27 (dd, J=3.2, 8.4 Hz, 1H), 7.18 (dd, J=2.0, 7.6 Hz, 1H), 7.10 (s, 2H), 6.47 (dd, J=4.8, 7.2 Hz, 1H), 2.14 (s, 3H). ¹⁹F NMR (376 MHz, DMSO-d₆) δ=−67.455.

Step 3. To a solution of 3-(3-(6-fluoro-2-methylpyridin-3-yl)-5-phenyl-3H-imidazo[4,5-b]pyridin-2-yl)pyridin-2-amine (270 mg, 0.681 mmol) in NMP (3 mL), methyl (1r,4r)-4-aminocyclohexane-1-carboxylate; hydrogen chloride (198 mg, 1.02 mmol) and DIEA (0.36 mL, 2.04 mmol) were added and the mixture was subjected to microwave irradiation at 150° C. for 7 hrs. Water (10 mL) was added to the reaction mixture and the whole extracted with EtOAc (20 mL×3). The organic layer was dried over Na₂SO₄ and concentrated in vacuum to provide the crude material. The crude was purified by flash column (0-80% of EtOAc in PE) to give methyl (1r,4r)-4-((5-(2-(2-aminopyridin-3-yl)-5-phenyl-3H-imidazo[4,5-b]pyridin-3-yl)-6-methylpyridin-2-yl)amino)cyclohexane-1-carboxylate (150 mg, 41.3% yield) as a yellow oil. ¹H NMR (DMSO-d₆ 400 MHz) δ_{H}=8.26 (d, J=8.4 Hz, 1H), 8.03-7.93 (m, 4H), 7.51-7.44 (m, 2H), 7.43-7.36 (m, 2H), 7.27 (dd, J=2.0, 8.0 Hz, 1H), 7.22 (s, 2H), 6.77 (d, J=7.6 Hz, 1H), 6.51-6.40 (m, 2H), 3.62-3.58 (m, 3H), 2.38-2.25 (m, 2H), 2.11-2.01 (m, 2H), 1.52-1.38 (m, 2H), 1.37-1.24 (m, 2H), 1.23-1.14 (m, 2H).

Step 4. To a solution of methyl (1r,4r)-4-((5-(2-(2-aminopyridin-3-yl)-5-phenyl-3H-imidazo[4,5-b]pyridin-3-yl)-6-methylpyridin-2-yl)amino)cyclohexane-1-carboxylate (150 mg, 0.281 mmol) in water (2 mL) and THF (2 mL), LiOH·H₂O (35 mg, 0.843 mmol) was added and the reaction stirred at 25° C. for 16 hrs. The crude was purified by prep-HPLC (column: Welch Xtimate C18 150*30 mm*5 μm; mobile phase:water(FA)-ACN; B %:20%-50%, 30 min) to give (1r,4r)-4-((5-(2-(2-aminopyridin-3-yl)-5-phenyl-3H-imidazo[4,5-b]pyridin-3-yl)-6-methylpyridin-2-yl)amino)cyclohexane-1-carboxylic acid I-121 (51.0 mg, 35.1% yield) as a yellow solid. ¹H NMR (DMSO-d₆ 400 MHz) δ_{H}=8.26 (d, J=8.4 Hz, 1H), 8.04-7.93 (m, 4H), 7.51-7.44 (m, 2H), 7.43-7.36 (m, 2H), 7.32-7.20 (m, 3H), 6.76 (d, J=7.6 Hz, 1H), 6.52-6.39 (m, 2H), 3.75-3.50 (m, 2H), 2.25-2.14 (m, 1H), 2.10-1.99 (m, 2H), 1.98-1.87 (m, 5H), 1.50-1.37 (m, 2H), 1.30-1.19 (m, 2H). HPLC Rₜ=3.527 min in 8 min chromatography, purity 100%. LCMS Rₜ=2.799 min in 4 min chromatography, purity 100%, MS ESI calcd. For 519.24 [M+H]⁺ 520.24, found 520.1.

### Example 113. Synthesis of N⁴-(5-(2-(2-aminopyridin-3-yl)-5-phenyl-31-imidazo[4,5-b]pyridin-3-yl)pyridin-2-yl)piperidine-1,4-dicarboxamide (Compound I-122)

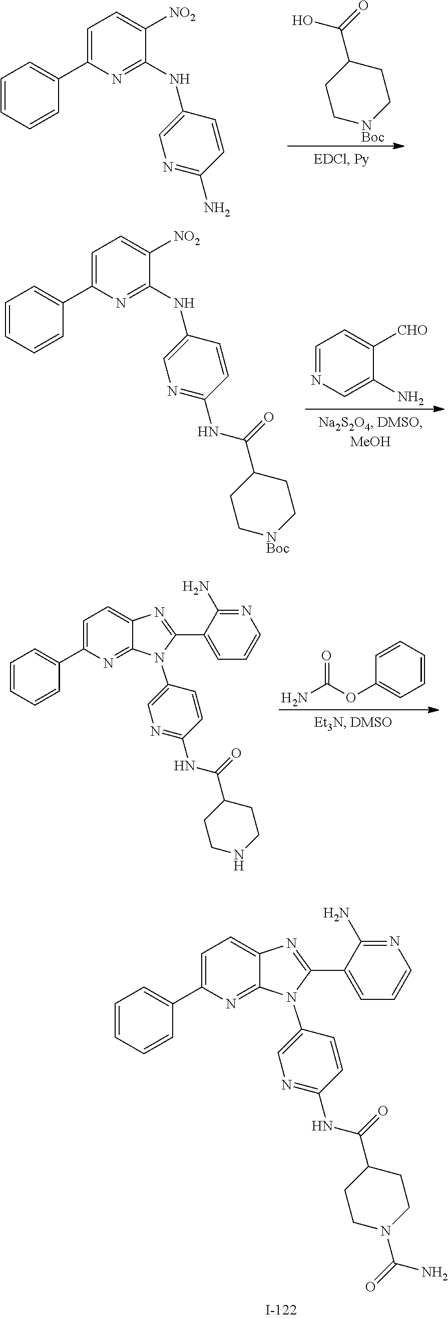

Step 1. A solution of N⁵-(3-nitro-6-phenyl-2-pyridyl)pyridine-2,5-diamine (1.00 g, 3.25 mmol), 1-tert-butoxycarbonylpiperidine-4-carboxylic acid (821 mg, 3.58 mmol) and EDCI (1.25 g, 6.51 mmol) in pyridine (10 mL) was stirred at 25° C. for 2 hrs. The reaction was taken up in EtOAc (50 mL) and the organics washed with brine (3×50 mL). The organics were then separated and dried (Na₂SO₄) before concentration to dryness. The crude was then purified by flash column chromatography eluting with 20% EtOAc in Isohexane to give tert-butyl 4-[[5-[(3-nitro-6-phenyl-2-pyridyl)amino]-2-pyridyl]carbamoyl]piperidine-1-carboxylate (1.20 g, 71.1% yield) as a yellow solid. ¹H NMR (DMSO-d₆, 400 MHz) δ_{H}=10.55 (s, 1H), 10.09 (s, 1H), 8.68 (d, J=2.4 Hz, 1H), 8.61 (d, J=8.8 Hz, 1H), 8.17-8.01 (m, 4H), 7.60 (d, J=8.8 Hz, 1H), 7.52 (dd, J=1.6, 5.2 Hz, 3H), 3.82 (d, J=13.2 Hz, 2H), 2.83-2.74 (m, 3H), 1.79-1.76 (m, 2H), 1.49 (dd, J=3.6, 12.0 Hz, 2H), 1.41 (s, 9H).

Step 2. To a solution of tert-butyl 4-[[5-[(3-nitro-6-phenyl-2-pyridyl)amino]-2-pyridyl]carbamoyl]piperidine-1-carboxylate (1.00 g, 1.93 mmol) in DMSO (10 mL) and methanol (2 mL), 2-aminopyridine-3-carbaldehyde (707 mg, 5.79 mmol) and Na₂S₂O₄ (2.01 g, 11.6 mmol) were added at 25° C. and the mixture was stirred at 100° C. for 10 hrs. The reaction was taken up in EtOAc (100 mL) and the organics washed with water (50 mL) and brine (50 mL×3). The organics were then separated and dried (Na₂SO₄) before concentration to dryness. The crude product was purified by prep-HPLC (column: Welch Xtimate C18 250*50 mm*10 μm; mobile phase: [water (FA)-ACN]; B %: 0%-30%, 20 min) to give N-[5-[2-(2-amino-3-pyridyl)-5-phenyl-imidazo[4,5-b]pyridin-3-yl]-2-pyridyl]piperidine-4-carboxamide (450 mg, 47.6% yield) as a white solid. ¹H NMR (DMSO-d₆, 400 MHz) δ_{H}=10.80 (s, 1H), 8.44 (d, J=2.4 Hz, 1H), 8.31-8.24 (m, 2H), 8.10-7.95 (m, 6H), 7.53-7.37 (m, 5H), 6.83 (br s, 2H), 6.52 (dd, J=4.8, 7.6 Hz, 1H), 2.87-2.76 (m, 4H), 1.94 (d, J=12.4 Hz, 2H), 1.85-1.69 (m, 3H).

Step 3. To a solution of N-[5-[2-(2-amino-3-pyridyl)-5-phenyl-imidazo[4,5-b]pyridin-3-yl]-2-pyridyl]piperidine-4-carboxamide (350 mg, 0.713 mmol) in DMSO (5 mL), TEA (0.40 mL, 2.85 mmol) and phenyl carbamate (117 mg, 0.856 mmol) were added under N₂ atmosphere and the mixture was stirred at 20° C. for 6 hrs. The reaction mixture was purified by prep-HPLC (column: Welch Xtimate C18 200*40 mm*7 μm; mobile phase: [water (FA)-ACN]; B %: 0%-40%, 25 min) to give N4-[5-[2-(2-amino-3-pyridyl)-5-phenyl-imidazo[4,5-b]pyridin-3-yl]-2-pyridyl]piperidine-1,4-dicarboxamide 1-122 (89.0 mg, 22.8% yield) as a yellow solid ¹H NMR (DMSO-d₆0.400 MHz), δ_{H}=10.73 (s, 1H), 8.42 (d, J=2.4 Hz, 1H), 8.27 (dd, J=8.4, 11.2 Hz, 2H), 8.08-7.94 (m, 5H), 7.51-7.44 (m, 2H), 7.43-7.34 (m, 2H), 6.83 (s, 2H), 6.52 (dd, J=4.8, 7.6 Hz, 1H), 5.93 (s, 2H), 3.99 (d, J=13.2 Hz, 2H), 2.74-2.64 (m, 3H), 1.76 (d, J=10.8 Hz, 2H), 1.49 (dq, J=4.0, 12.4 Hz, 2H). HPLC Rₜ=2.196 in 8 min chromatography, ACE Excel 3 C18 4 6*100 mm, purity 97.6%. LCMS Rₜ=1.547 min in 4 min chromatography, Agilent PoroShell 120 EC-C18 2.7 um 3.0*50 mm, purity 98.48%, MS ESI calcd. for 533.23 [M+H]⁺ 534.23, found 534.3.

### Example 114. Synthesis of (1r,4r)-4-((5-(2-(2-aminopyridin-3-yl)-6-(trifluoromethoxy)-1H-benzo[d]imidazol-1-yl)pyridin-2-yl)carbamoyl)cyclohexane-1-carboxylic acid (Compound I-123)

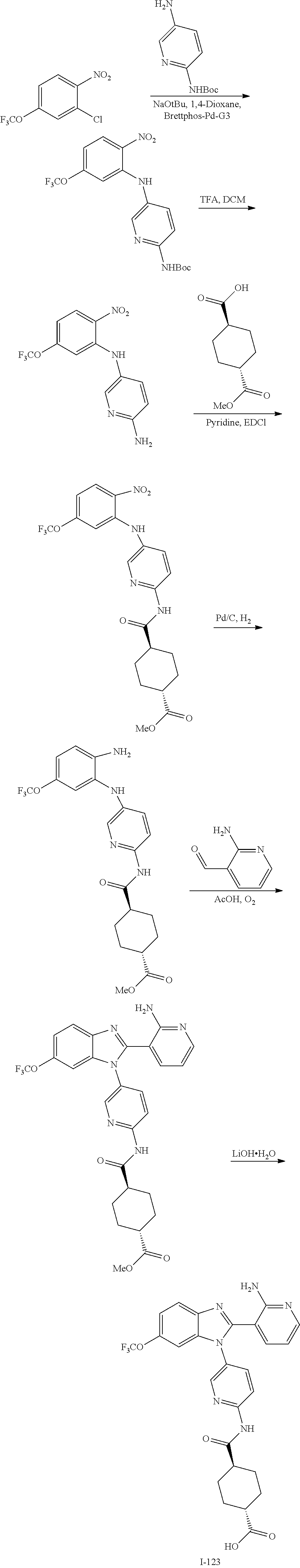

Step 1. To a solution of tert-butyl (5-aminopyridin-2-yl)carbamate (2.43 g, 11.6 mmol) in 1,4-dioxane (60 mL), 2-chloro-1-nitro-4-(trifluoromethoxy)benzene (2.00 g, 8.28 mmol), t-BuONa (2.39 g, 24.8 mmol) and BrettPhos-Pd-G3 (750 mg, 0.828 mmol) were added at 25° C. The reaction mixture was degassed and purged with N₂ (×3) and the mixture was stirred at 80° C. for 14 hrs under N₂. Water (60 mL) was added to the residue and the resulting mixture was extracted with EtOAc (40 mL×3). The combined organic phase was washed with brine (50 mL), dried over anhydrous Na₂SO₄, filtered and concentrated. The residue was purified by flash silica gel chromatography (eluent of 0-5% EtOAc in PE) to give tert-butyl (5-((2-nitro-5-(trifluoromethoxy)phenyl)amino)pyridin-2-yl)carbamate (1.60 g, 46.6% yield) as a yellow solid. ¹H NMR (DMSO-d₆. 400 MHz) β_{H}=9.90 (s, 1H), 9.54 (s, 1H), 8.20-8.32 (m, 2H), 7.87 (d, J=8.8 Hz, 1H), 7.73 (dd, J=8.8, 2.4 Hz, 1H), 6.80 (d, J=9.2 Hz, 1H), 6.70 (s, 1H), 1.48 (s, 9H).

Step 2. To a solution of tert-butyl (5-((2-nitro-5-(trifluoromethoxy)phenyl)amino)pyridin-2-yl)carbamate (500 mg, 1.20 mmol) in DCM (10 mL), TFA (137 mg, 1.20 mmol) was added and the mixture was stirred at 25° C. for 12 hrs. The reaction mixture was concentrated directly to give N⁵-(2-nitro-5-(trifluoromethoxy) phenyl) pyridine-2,5-diamine (400 mg, 94.9% yield) as a yellow oil, which was used in the next step directly. LCMS Rₜ=0.391 min in 1 min chromatography, purity 90.5%, MS ESI calcd. for 314.22 [M+H]⁺ 315.22, found 315.00.

Step 3. To a solution of N-(2-nitro-5-(trifluoromethoxy) phenyl) pyridine-2,5-diamine (800 mg, 2.55 mmol) and (1r,4r)-4-(methoxycarbonyl)cyclohexane-1-carboxylic acid (474 mg. 2.55 mmol) in pyridine (15 mL), EDCI (730 mg, 3.82 mmol) was added. Water (15 mL) was added to the residue and the resulting mixture was extracted with EtOAc (30 mL×3). The combined organic phase was washed with brine (15 mL), dried over anhydrous Na₂SO₄, filtered and concentrated The residue was purified by flash silica gel chromatography(eluent of 0-20% EtOAc in PE) to give methyl (1r,4r)-4-((5-((2-nitro-5-(trifluoromethoxy)phenyl)amino)pyridin-2-yl)carbamoyl)cyclohexane-1-carboxylate (220 mg, 0.456 mmol, 17.9% yield) as a light yellow solid. ¹H NMR (400 MHz, DMSO-d₆) δ=10.52 (s, 1H), 9.56 (s, 1H), 8.30-8.25 (m, 2H), 8.16 (d, J=8.8 Hz, 1H), 7.76 (dd, J=2.4, 8.8 Hz, 1H), 6.84-6.73 (m, 2H), 3.60 (s, 3H), 2.54 (s, 1H), 2.36-2.29 (m, 1H), 1.99-1.86 (m, 4H), 1.54-1.27 (m, 5H).

Step 4. To a solution of methyl (1r,4r)-4-((5-((2-nitro-5-(trifluoromethoxy)phenyl)amino)pyridin-2-yl)carbamoyl)cyclohexane-1-carboxylate (400 mg, 0.840 mmol) in THF (10 mL), wet Pd/C (100 mg, purity 10%) was added under N₂. The reaction mixture was degassed and purged with H₂ (15 psi) (×3) and the mixture was stirred at 25° C. for 12 hrs under H₂ (15 psi). The reaction mixture was filtered, and the filtrate was concentrated directly to give methyl (1r,4r)-4-((5-((2-amino-5-(trifluoromethoxy)phenyl)amino)pyridin-2-yl)carbamoyl)cyclohexane-1-carboxylate (260 mg, crude) as a yellow solid, which was used in the next step directly. ¹H NMR (400 MHz, DMSO-d₆) δ=10.77 (s, 1H), 8.13-8.09 (m, 2H), 8.05-8.03 (m, 2H), 8.02-7.95 (m, 2H), 7.48 (s, 1H), 7.26 (dd, J=2.8, 8.8 Hz, 1H), 6.94-6.89 (m, 1H), 6.81-6.78 (m, 1H), 3.91-3.89 (m, 3H).

Step 5. To a solution of methyl (1r,4r)-4-((5-((2-amino-5-(trifluoromethoxy)phenyl)amino)pyridin-2-yl)carbamoyl)cyclohexane-1-carboxylate (180 mg, 0.398 mmol) in acetic acid (5 mL), 2-aminonicotinaldehyde (58.0 mg, 0.477 mmol) was added under 02. The reaction mixture was degassed and purged with 02 (×3) and the mixture was stirred at 80° C. for 1 hr under 02. The reaction mixture was concentrated directly to give methyl (1r,4r)-4-((5-(2-(2-aminopyridin-3-yl)-6-(trifluoromethoxy)-1H-benzo[d]imidazol-1-yl)pyridin-2-yl)carbamoyl)cyclohexane-1-carboxylate (240 mg, 97.9% yield) as a light yellow oil, which was used in the next step directly. ¹H NMR (400 MHz, DMSO-d₆) δ=10.69 (s, 1H), 8.34 (d, J=2.4 Hz, 1H), 8.26-8.24 (m, 1H), 7.99 (dd, J=1.6, 4.8 Hz, 1H), 7.94 (dd, J=2.8, 8.8 Hz, 1H), 7.91 (d, J=8.8 Hz, 1H), 7.35-7.25 (m, 3H), 6.77-6.71 (m, 2H), 6.47 (dd, J=4.8, 7.6 Hz, 1H), 3.60 (s, 3H), 2.53-2.52 (m, 1H), 2.36-2.31 (m, 1H), 2.03-1.93 (m, 3H), 1.88 (s, 1H), 1.50-1.42 (m, 2H), 1.41-1.34 (m, 2H).

Step 6. To a solution of methyl (1r,4r)-4-((5-(2-(2-aminopyridin-3-yl)-6-(trifluoromethoxy)-1H-benzo[d]imidazol-1-yl)pyridin-2-yl)carbamoyl)cyclohexane-1-carboxylate (240 mg, 0.433 mmol) in THF (3 mL) and water (3 mL), LiOH·H₂O (22.0 mg, 0.519 mmol) was added at 40° C. and the mixture was stirred at 40° C. for 2 hrs. The mixture was filtered directly. The residue was purified by prep-HPLC (Column: Welch Xtimate C18 150*30 mm*5 um; mobile phase: [water(NH₄HCO₃)-ACN]; B %: 2%-42%; 25 min) to afford (1r,4r)-4-((5-(2-(2-aminopyridin-3-yl)-6-(trifluoromethoxy)-1H-benzo[d]imidazol-1-yl)pyridin-2-yl)carbamoyl)cyclohexane-1-carboxylic acid I-123 (100 mg, 42.5% yield) as a light yellow solid. ¹H NMR (400 MHz, DMSO-d₆) δ=10.69 (s, 1H), 8.38-8.22 (m, 2H), 8.03-7.87 (m, 3H), 7.38-7.21 (m, 3H), 6.75 (s, 2H), 6.48 (dd, J=4.8, 7.6 Hz, 1H), 2.54-2.52 (m, 1H), 2.24-2.14 (m, IH), 1.99-1.86 (m, 4H), 1.52-1.41 (m, 2H), 1.38-1.27 (m, 2H). HPLC Rₜ=2.448 min in 8 min chromatography, purity 99.2%. LCMS Rₜ=1.160 min in 2 min chromatography, purity 99.1%, MS ESI calcd. for 540.50 [M+H]⁺ 541.50, found 541.10.

### Example 115. Synthesis of (1r,4r)-4-((5-(2-(2-aminopyridin-3-yl)-6-isopropoxy-1H-benzo[d]imidazol-1-yl)pyridin-2-yl)carbamoyl)cyclohexane-1-carboxylic acid (Compound I-124)

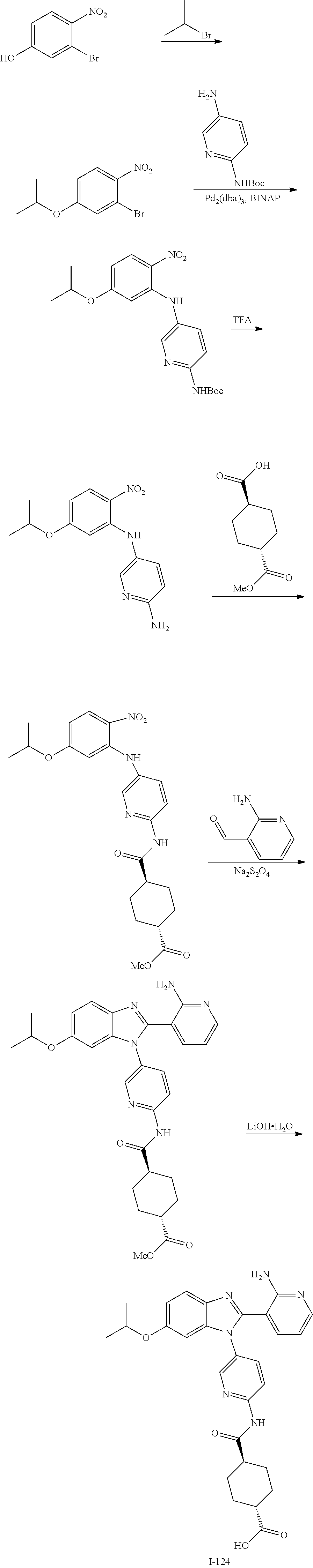

Step 1. To a solution of 3-bromo-4-nitro-phenol (2.00 g, 9.17 mmol) in DMF (30 mL), K₂CO₃ (3.80 g, 27.5 mmol) and 2-bromopropane (2.26 g, 18.3 mmol) were added and the reaction stirred at 90° C. for 16 hrs. Water (100 mL) was added to the reaction mixture and the whole extracted with EtOAc (60 mL×3). The organic layer was dried over Na₂SO₄ and concentrated in vacuum to give a crude. The crude was purified by flash column (0-50% of EtOAc in PE) to give 2-bromo-4-isopropoxy-1-nitro-benzene (2.00 g, 83.8% yield) as a yellow oil.

Step 2. To a solution of 2-bromo-4-isopropoxy-1-nitro-benzene (2.30 g, 8.84 mmol), t-BuONa (2.55 g, 26.5 mmol) and BINAP (700 mg, 1.13 mmol) in toluene (50 mL), Pd₂(dba)₃ (460 mg, 0.800 mmol) was added and the reaction mixture stirred at 80° C. for 1 hr. The reaction mixture was concentrated and subsequently diluted with EtOAc (15 mL), washed with water (20 mL) and brine (20 mL×2), dried over anhydrous sodium sulfate, filtered and the filtrate was concentrated to afford a residue. The crude product was purified by flash chromatography on silica gel (20% of EtOAc in PE) to afford tert-butyl N-[5-(5-isopropoxy-2-nitro-anilino)-2-pyridyl]carbamate (1.00 g, 87.3% yield) as a yellow solid. ¹H NMR (400 MHz, DMSO-d₆) δ=9.89 (s, 1H), 9.53 (s, 1H), 8.24 (d, J=2.4 Hz, 1H), 8.10 (d, J=9.6 Hz, 1H), 7.86 (d, J=9.2 Hz, 1H), 7.75 (dd, J=2.4, 8.8 Hz, 1H), 6.46 (dd, J=2.4, 9.6 Hz, 1H), 6.19 (d, J=2.4 Hz, 1H), 4.55 (q, J=6.0 Hz, 1H), 1.48 (s, 9H), 1.21 (d, J=6.0 Hz, 6H).

Step 3. To a solution of tert-butyl N-[5-(5-isopropoxy-2-nitro-anilino)-2-pyridyl]carbamate (1.20 g, 3.09 mmol) in DCM (6 mL), TFA (6.0 mL, 3.09 mmol) was added at 25° C. and the mixture was stirred for 5 hrs. The mixture was concentrated under reduced pressure to give N⁵-(5-isopropoxy-2-nitro-phenyl)pyridine-2,5-diamine (1.50 g, crude) as a yellow solid, which was directly used in the next step according to the theoretical amount. ¹H NMR (400 MHz, DMSO-d₆) δ=9.38 (s, 1H), 8.27-8.14 (m, 1H), 8.11 (d, J=9.6 Hz, 1H), 8.00 (s, 1H), 7.94 (dd, J=2.0, 9.6 Hz, 1H), 7.03 (d, J=9.6 Hz, 1H), 6.50 (dd, J=2.4, 9.6 Hz, 1H), 6.20 (d, J=2.4 Hz, 1H), 4.66 (q, J=6.0 Hz, 1H), 1.22 (d, J=6.0 Hz, 6H).

Step 4. To a solution of N⁵-(5-isopropoxy-2-nitro-phenyl)pyridine-2,5-diamine (350 mg, 1.21 mmol) in pyridine (1.50 mL), (1r,4r)-4-(methoxycarbonyl)cyclohexane-1-carboxylic acid (271 mg, 1.46 mmol) and EDCI (348 mg, 1.82 mmol) were added at 25° C. under N₂ and the mixture was stirred for 16 hrs. Water (20 mL) was added, and the mixture was extracted with EtOAc (20 mL×3). The organic phase was washed with brine (20 mL), dried over Na₂SO₄, filtered and concentrated under reduced pressure. The crude was purified by flash chromatography eluting with PE/EtOAc (from 0% to 30% of EtOAc in PE) to give methyl (1r,4r)-4-((5-((5-isopropoxy-2-nitrophenyl)amino)pyridin-2-yl)carbamoyl)cyclohexane-1-carboxylate (112 mg, 20.2% yield) as a yellow oil. 1H NMR (400 MHz, DMSO-d₆) δ=10.50 (s, IH), 9.55 (s, 1H), 8.30 (d, J=2.8 Hz, 1H), 8.18-8.08 (m, 2H), 7.79 (dd, J=2.4, 8.8 Hz, 1H), 6.46 (dd, J=2.4, 9.6 Hz, 1H), 6.23 (d, J=2.4 Hz, 1H), 4.56 (q, J=6.0 Hz, 1H), 3.60 (s, 3H), 2.35-2.30 (m, 1H), 1.96-1.86 (m, 4H), 1.53-1.42 (m, 2H), 1.38-1.30 (m, 3H), 1.21 (d, J=6.0 Hz, 6H).

Step 5. To a solution of methyl (1r,4r)-4-((5-((5-isopropoxy-2-nitrophenyl)amino)pyridin-2-yl)carbamoyl)cyclohexane-1-carboxylate (130 mg, 0.285 mmol) in DMSO (4 mL) and methanol (1 mL), 2-aminopyridine-3-carbaldehyde (42.0 mg, 0.342 mmol) and Na₂S₂O₄ (124 mg, 0.712 mmol) were added at 25° C. and the mixture was stirred at 100° C. for 16 hrs. Water (20 ml) was added and the mixture was extracted with EtOAc (20 mL×3). The organic phase was washed with brine (20 mL×3), dried over Na₂SO₄, filtered, and concentrated under reduced pressure to give a crude. The crude was purified by flash chromatography eluting with PE/EtOAc (from 0% to 100% of EtOAc in PE) to give methyl (1r,4r)-4-((5-(2-(2-aminopyridin-3-yl)-6-isopropoxy-1H-benzo[d]imidazol-1-yl)pyridin-2-yl)carbamoyl)cyclohexane-1-carboxylate (120 mg, 79.7% yield) as a yellow solid. ¹H NMR (400 MHz, DMSO-d₆) δ=8.45-8.39 (m, 1H), 8.20 (d, J=2.6 Hz, 1H), 8.10 (s, 1H), 8.03 (dd, J=1.6, 5.2 Hz, 1H), 7.71-7.69 (m, 1H), 7.04-6.94 (m, 2H), 6.63 (d, J=2.4 Hz, 1H), 6.49 (s, 2H), 6.39 (dd, J=5.2, 7.6 Hz, 1H), 4.57-4.47 (m, 1H), 3.70 (s, 3H), 2.40-2.28 (m, 2H), 2.13 (d, J=2.8 Hz, 2H), 1.71-1 50 (m, 6H), 1.31 (d, J=6.4 Hz, 6H).

Step 6. To a solution of methyl (1r,4r)-4-((5-(2-(2-aminopyridin-3-yl)-6-isopropoxy-1H-benzo[d]imidazol-1-yl)pyridin-2-yl)carbamoyl)cyclohexane-1-carboxylate (120 mg, 0.227 mmol) in THF (1 mL) and water (1 mL), LiOH·H₂O (24.0 mg, 0.568 mmol) was added at 25° C. and the mixture was stirred for 16 hrs. The mixture was concentrated under reduced pressure to provide the crude material. The crude was purified by prep-HPLC (Welch Xtimate C18 150*30 mm*5 um water(FA)-ACN) to give (1r,4r)-4-((5-(2-(2-aminopyridin-3-yl)-6-isopropoxy-1H-benzo[d]imidazol-1-yl)pyridin-2-yl)carbamoyl)cyclohexane-1-carboxylic acid I-124 (43.0 mg, 36.6% yield) as a yellow solid. ¹H NMR (400 MHz, DMSO-d₆) δ=10.68 (s, 1H), 8.29 (d, J=2.4 Hz, 1H), 8.26 (d, J=8.8 Hz, 1H), 7.95 (dd, J=1.6, 4.8 Hz, 1H), 7.91 (dd, J=2.4, 8.8 Hz, 1H), 7.67 (d, J=8.8 Hz, 1H), 7.17 (dd, J=1.6, 7.6 Hz, 1H), 6.93 (dd, J=2.4, 8.8 Hz, 1H), 6.86 (s, 2H), 6.68 (d, J=2.0 Hz, 1H), 6.43 (dd, J=5.2, 7.6 Hz, 1H), 4.58 (td, J=6.0, 12.0 Hz, 1H), 2.49-2.45 (m, 1H), 2.25-2.16 (m, 1H), 1.99-1.85 (m, 4H), 1.52-1.41 (m, 2H), 1.38-1.28 (m, 2H), 1.22 (d, J=6.0 Hz, 6H). HPLC Rₜ=2.778 min in 8.0 min chromatography, 10-80AB_8 min.1 cm, purity 100%. LCMS Rₜ=2.374 min in 4.0 min chromatography, purity 98.1%, MS ESI calcd. for 514.23, [M+H]⁺ 515.23, found 515.1.

### Example 116. Synthesis of 6-((5-(2-(2-aminopyridin-3-yl)-5-phenyl-3H-imidazo[4,5-b]pyridin-3-yl)-6-methylpyridin-2-yl)carbamoyl)spiro[3.3]heptane-2-carboxylic acid (Compound I-125)

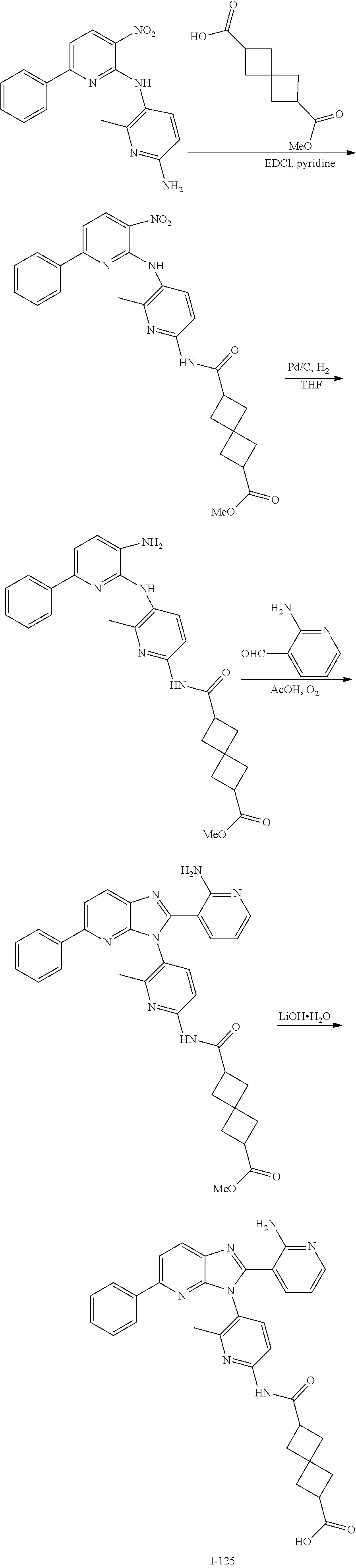

Step 1. To a solution of 6-methyl-N⁵-(3-nitro-6-phenylpyridin-2-yl)pyridine-2,5-diamine (250 mg, 0.778 mmol) and 2-methoxycarbonylspiro[3.3]heptane-6-carboxylic acid (154 mg, 0.778 mmol) in pyridine (10 mL), EDCI (444 mg, 1.17 mmol) was added and the mixture was stirred at 60° C. for 2 hrs. Water (10 mL) was added to the residue and the resulting mixture was extracted with EtOAc (10 mL×3). The combined organic phase was washed with brine (20 mL), dried over anhydrous Na₂SO₄, filtered and concentrated. The residue was purified by flash silica gel chromatography (eluent of 0-30% EtOAc in PE) to give methyl 6-((6-methyl-5-((3-nitro-6-phenylpyridin-2-yl)amino)pyridin-2-yl)carbamoyl)spiro[3.3]heptane-2-carboxylate (140 mg, 0.279 mmol) as a yellow oil. ¹H NMR (400 MHz, DMSO-d₆) δ=10.34 (s, 1H), 9.93 (s, 1H), 8.60 (d, J=8.8 Hz, 1H), 8.06-7.97 (m, 2H), 7.95-7.92 (m, 2H), 7.55 (d, J=8.8 Hz, 1H), 7.50-7.46 (m, 3H), 3.59 (s, 3H), 3.09-2.97 (m, 2H), 2 69 (s, 3H), 2 28-2 222 (m, 4H), 2.17-2.09 (m, 4H).

Step 2. To a solution of methyl 6-((6-methyl-5-((3-nitro-6-phenylpyridin-2-yl)amino)pyridin-2-yl)carbamoyl)spiro[3.3]heptane-2-carboxylate (140 mg, 0.279 mmol) in THF (10 mL), Pd/C (100 mg, purity 10%) was added under N₂. The reaction mixture was degassed and purged with H₂ (15 psi) (×3) and the mixture was stirred at 25° C. for 12 hrs under H₂ (15 PSI). The reaction mixture was filtered and the filtrate was concentrated directly to give methyl 6-((5-((3-amino-6-phenylpyridin-2-yl)amino)-6-methylpyridin-2-yl)carbamoyl)spiro[3.3]heptane-2-carboxylate (110 mg, crude) as a light yellow oil, which was used in the next step directly. LCMS Rₜ=0.473 min in 1 min chromatography, purity 80.1%, MS ESI calcd. for 471.23, [M+H]⁺ 472.23, found 472.2.

Step 3. To a solution of methyl 6-((5-((3-amino-6-phenylpyridin-2-yl)amino)-6-methylpyridin-2-yl)carbamoyl)spiro[3.3]heptane-2-carboxylate (100 mg, 0.212 mmol) in acetic acid (3.00 mL), 2-aminopyridine-3-carbaldehyde (31 0 mg, 0.254 mmol) was added under 02 and the mixture was stirred at 80° C. for 1 hr under O₂. The reaction mixture was concentrated directly. The residue was purified by flash silica gel chromatography (eluent of 0-5% MeOH in DCM) to give methyl 6-((5-(2-(2-aminopyridin-3-yl)-5-phenyl-3H-imidazo[4,5-b]pyridin-3-yl)-6-methylpyridin-2-yl)carbamoyl)spiro[3.3]heptane-2-carboxylate (150 mg, crude) as a light yellow oil. LCMS Rₜ=0.496 min in 1 min chromatography, purity 76.6%, MS ESI calcd. for 573.25, [M+H]⁺ 574.25, found 574.2.

Step 4. To a solution of methyl 6-((5-(2-(2-aminopyridin-3-yl)-5-phenyl-3H-imidazo[4,5-b]pyridin-3-yl)-6-methylpyridin-2-yl)carbamoyl)spiro[3.3]heptane-2-carboxylate (100 mg, 0.174 mmol) in THF (4 mL) and water (4 mL), LiOH·H₂O (22 mg, 0.523 mmol) was added and the mixture was stirred at 25° C. for 2 hrs. The reaction mixture was concentrated directly The residue was purified by prep-HPLC (Column: Welch Xtimate C18 150*30 mm*5 μm; mobile phase: [water (NH₄HCO₃)-ACN]; B %: 0%-35%; 25 min) to give 6-((5-(2-(2-aminopyridin-3-yl)-5-phenyl-3H-imidazo[4,5-b]pyridin-3-yl)-6-methylpyridin-2-yl)carbamoyl)spiro[3.3]heptane-2-carboxylic acid I-125 (56.9 mg, 57.5% yield) as a light yellow solid. ¹H NMR (DMSO-d₆, 400 MHz) δ_{H}=10.58 (s, 1H), 8.30 (d, J=8.4 Hz, 1H), 8.12 (d, J=8.8 Hz, 1H), 8.06-7.96 (m, 4H), 7.86 (d, J=8.8 Hz, 1H), 7.52-7.33 (m, 3H), 7.23-7.11 (m, 3H), 6.46 (dd, J=4.8, 7.6 Hz, 1H), 3.26-3.23 (m, 1H), 2.97-2.86 (m, 1H), 2.28-2.08 (m, 8H), 2.06 (s, 3H). HPLC Rₜ 2.655 min and 2.740 min in 8 min chromatography, purity 98.6%. LCMS Rₜ=1.237 min and 1.272 min in 2 min chromatography, purity 95.1%, MS ESI calcd. for 559 23, [M+H]⁺ 560.23, found 560.1.

### Example 117. Synthesis of 6-((6-(2-(2-aminopyridin-3-yl)-5-phenyl-3H-imidazo[4,5-b]pyridin-3-yl)-2-methylpyridin-3-yl)carbamoyl)spiro[3.3]heptane-2-carboxylic acid (Compound I-126)

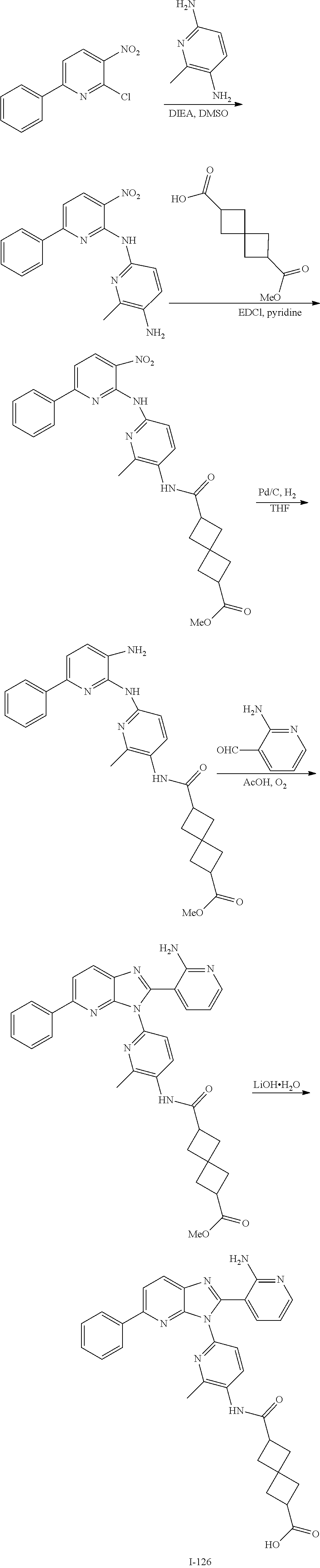

Step 1. To a solution of 2-chloro-3-nitro-6-phenylpyridine (4.00 g, 17.0 mmol) and 6-methylpyridine-2,5-diamine (2.52 g, 20.5 mmol) in DMSO (50 mL), DIEA (8.5 mL, 51.1 mmol) was added and the mixture was stirred at 80° C. for 12 hrs. Water (60 mL) was added to the residue and the resulting mixture was extracted with EtOAc (60 mL×3). The combined organic phase was washed with brine (60 mL), dried over anhydrous Na₂SO₄ filtered and concentrated. The residue was purified by flash silica gel chromatography (eluent of 0-10% MeOH in DCM) to give 6-methyl-N2-(3-nitro-6-phenylpyridin-2-yl)pyridine-2,5-diamine (5.38 g, crude) as a yellow solid. LCMS Rₜ=0 549 min in 1 min chromatography, purity 91.0%, MS ESI calcd. for 321.34, [M+H]⁺ 322.34, found 322.0.

Step 2. To a solution of 6-methyl-N2-(3-nitro-6-phenylpyridin-2-yl)pyridine-2,5-diamine (250 mg, 0.778 mmol) and 2-methoxycarbonylspiro[3.3]heptane-6-carboxylic acid (154 mg, 0.778 mmol) in pyridine (10 mL), EDCl (444 mg, 1.17 mmol) was added and the mixture was stirred at 60° C. for 2 hrs. Water (10 mL) was added to the residue and the resulting mixture was extracted with EtOAc (10 mL×3). The combined organic phase was washed with brine (20 mL), dried over anhydrous Na₂SO₄, filtered and concentrated. The residue was purified by flash silica gel chromatography (eluent of 0-75% EtOAc in PE) to give methyl 6-((2-methyl-6-((3-nitro-6-phenylpyridin-2-yl)amino)pyridin-3-yl)carbamoyl)spiro[3.3]heptane-2-carboxylate (400 mg, 0.798 mmol) as a yellow oil. ¹H NMR (DMSO-d₆, 400 MHz) δ_{H}=10.42 (s, 1H), 9.31 (s, 1H), 8.64 (d, J=8.8 Hz, 1H), 8.22-8 13 (m, 3H), 7.88 (d, J=8.8 Hz, 1H), 7.68 (d, J=8.8 Hz, 1H), 7.62-7.56 (m, 3H), 3.59 (s, 3H), 3.22-3.11 (m, 1H), 3.09-2.98 (m, 1H), 2.33 (s, 3H), 2.31-2.08 (m, 8H).

Step 3. To a solution of methyl 6-((2-methyl-6-((3-nitro-6-phenylpyridin-2-yl)amino)pyridin-3-yl)carbamoyl)spiro[3.3]heptane-2-carboxylate (400 mg, 0.798 mmol) in THF (10 mL), Pd/C; (200 mg, purity 10%) was added under N₂. The reaction mixture was degassed and purged with H₂ (30 psi) (×3) and the mixture was stirred at 25° C. for 12 hrs under H₂ (30 psi). The reaction mixture was filtered and the filtrate was concentrated directly to give methyl 6-((6-((3-amino-6-phenylpyridin-2-yl)amino)-2-methylpyridin-3-yl)carbamoyl)spiro[3.3]heptane-2-carboxylate (310 mg, 0.657 mmol) as a light yellow oil, which was used in the next step directly. ¹H NMR (DMSO-d₆, 400 MHz) δ_{H}=9.18 (s, 1H), 8.41 (s, 1H), 8.07-7.90 (m, 3H), 7.59 (d, J=8.8 Hz, 1H), 7.43-7.24 (m, 4H), 7.02 (d, J=8.0 Hz, 1H), 5.41 (s, 2H), 3.59 (s, 3H), 3.16-2.99 (m, 2H), 2 33-2.23 (m, 7H), 2.20-2.08 (m, 4H).

Step 4. To a solution of methyl 6-((6-((3-amino-6-phenylpyridin-2-yl)amino)-2-methylpyridin-3-yl)carbamoyl)spiro[3.3]heptane-2-carboxylate (310 mg, 0.657 mmol) in acetic acid (3 mL), 2-aminopyridine-3-carbaldehyde (96.0 mg, 0.789 mmol) was added under O₂ and the mixture was stirred at 80° C. for 1 hr under 02. The reaction mixture was concentrated directly. The residue was purified by flash silica gel chromatography (eluent of 0-5% MeOH in DCM) to give methyl 6-((6-(2-(2-aminopyridin-3-yl)-5-phenyl-3H-imidazo[4,5-b]pyridin-3-yl)-2-methylpyridin-3-yl)carbamoyl)spiro[3.3]heptane-2-carboxylate (350 mg, 0.574 mmol) as a yellow oil. LCMS Rₜ=0.603 min in 1 min chromatography, purity 94.0%, MS ESI calcd. for 573.25, [M+H]⁺ 574.25, found 574.3.

Step 5. To a solution of methyl 6-((6-(2-(2-aminopyridin-3-yl)-5-phenyl-3H-imidazo[4,5-b]pyridin-3-yl)-2-methylpyridin-3-yl)carbamoyl)spiro[3.3]heptane-2-carboxylate (350 mg, 0.610 mmol) in THF (4 mL) and water (4 mL), LiOH·H₂O (77.0 mg. 1.83 mmol) was added and the mixture was stirred at 25° C. for 4 hrs. The reaction mixture was concentrated directly. The residue was purified by prep-HPLC (Column: Welch Xtimate C18 150*30 mm*5 μm; mobile phase: [water (NH₄HCO₃)-ACN]; B %: 0%-35%; 25 min) to give 6-((6-(2-(2-aminopyridin-3-yl)-5-phenyl-3H-imidazo[4,5-b]pyridin-3-yl)-2-methylpyridin-3-yl)carbamoyl)spiro[3.3]heptane-2-carboxylic acid I-126 (194.4 mg, 54.5% yield) as a light yellow solid. ¹H NMR (DMSO-d₆, 400 MHz) δ_{H}=9.43 (s, 1H), 8.31-8.22 (m, 2H), 8.07-7.98 (m, 4H), 7.75 (d, J=8.4 Hz, 1H), 7.50-7.37 (m, 3H), 7.19-7.04 (m, 3H), 6.43 (dd, J=4.8, 7.6 Hz, 1H), 3.26-3.21 (m, 1H), 2.98-2.88 (m, 1H), 2.30-2.09 (m, 1H). HPLC Rₜ=2.479 min in 8 min chromatography, purity 95.7%. LCMS Rₜ=1.183 min in 2 min chromatography, purity 88.1%, MS ESI calcd. for 559.23, [M+H]⁺ 560.23, found 560.2.

### Example 718. Synthesis of (1r,4r)-4-((5-(2-(2-aminopyridin-3-yl)-6-phenyl-1H-benzo[d]imidazol-1-yl)-6-methylpyridin-2-yl)carbamoyl)cyclohexane-1-carboxylic acid (Compound I-127)

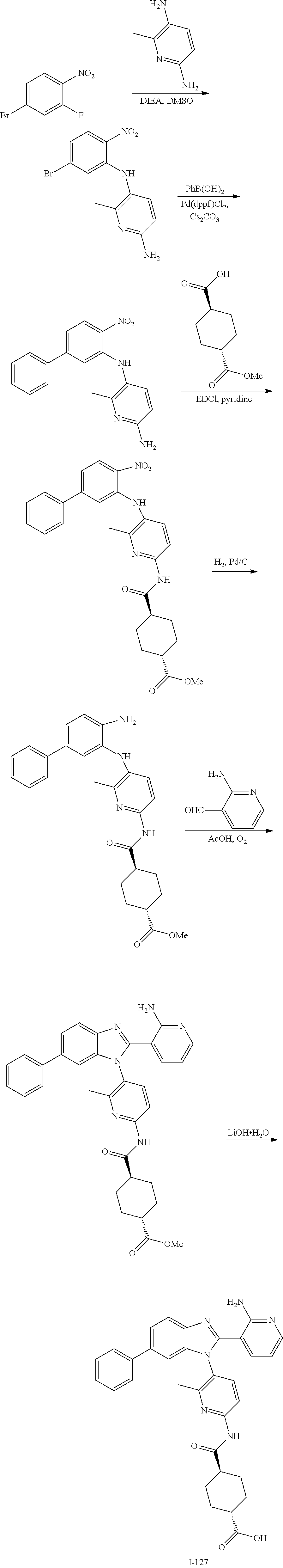

Step 1. To a solution of 4-bromo-2-fluoro-1-nitro-benzene (500 mg, 2.27 mmol) in DMSO (5 mL), 6-methylpyridine-2,5-diamine (280 mg, 2.27 mmol) and DIEA (881 mg, 6.82 mmol) were added at 25° C. and the mixture was stirred at 80° C. for 12 hrs. The reaction mixture was concentrated. Water (5 mL) was added to the residue and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent of 20-30% EtOAc in PE) to give N5-(5-bromo-2-nitrophenyl)-6-methylpyridine-2,5-diamine (270 mg, 36.8% yield) as a yellow solid. ¹H NMR (DMSO-d₆, 400 MHz) β_{H}=9.25 (s, 1H), 8.03 (d, J=9.2 Hz, 1H), 7.23 (d, J=8.4 Hz, 1H), 6.94-6.85 (m, 1H), 6.58 (d, J=2.0 Hz, 1H), 6.38 (d, J=8.4 Hz, 1H), 6.08 (s, 2H), 2.11 (s, 3H).

Step 2. To a solution of N5-(5-bromo-2-nitrophenyl)-6-methylpyridine-2,5-diamine (270 mg, 0.836 mmol) in 1,4-dioxane (10 mL) and water (2 mL), phenylboronic acid (204 mg, 1.67 mmol), Cs₂CO₃ (817 mg, 2.51 mmol) and Pd(dppf)Cl₂ (61.0 mg, 0.0836 mmol) were added at 25° C., and the mixture was stirred at 90° C. for 12 hrs. Water (15 mL) was added to the residue and the resulting mixture was extracted with EtOAc (20 mL×3). The combined organic phase was washed with brine (20 mL), dried over anhydrous Na₂SO₄, filtered and concentrated. The reaction mixture was concentrated directly The residue was purified by flash silica gel chromatography (eluent of 35-40% EtOAc in PE) to give 6-methyl-N5-(4-nitro-[1,1′-biphenyl]-3-yl)pyridine-2,5-diamine (170 mg, 63.5% yield) as a red solid. ¹H NMR (DMSO-d₆, 400 MHz) δ_{H}=9.28 (s, 1H), 8.20 (d, J=8.8 Hz, 1H), 7.49-7.41 (m, 5H), 7.30 (d, J=8.4 Hz, 1H), 7.04 (dd, J=1.6, 8.8 Hz, 1H), 6.66 (d, J=1.6 Hz, 1H), 6.38 (d, J=8.4 Hz, 1H), 6.02 (s, 2H), 2.16 (s, 3H).

Step 3. To a solution of 6-methyl-N5-(4-nitro-[1,1′-biphenyl]-3-yl)pyridine-2,5-diamine (120 mg, 0.375 mmol) and (1r,4r)-4-(methoxycarbonyl)cyclohexane-1-carboxylic acid (70.0 mg, 0.375 mmol) in pyridine (5 mL), EDCI (107 mg, 0.562 mmol) was added and the mixture was stirred at 80° C. for 12 hrs. Water (5 mL) was added to the residue and the resulting mixture was extracted with EtOAc (10 mL×3). The combined organic phase was washed with brine (5 mL), dried over anhydrous Na₂SO₄, filtered and concentrated. The residue was purified by flash silica gel chromatography (eluent of 20-30% EtOAc in PE) to give methyl (1r,4r)-4-((6-methyl-5-((4-nitro-[1,1′-biphenyl]-3-yl)amino)pyridin-2-yl)carbamoyl)cyclohexane-1-carboxylate (140 mg, 76.5% yield) as a yellow solid. ¹H NMR (DMSO-d₆, 400 MHz) δ_{H}=10.49 (s, 1H), 9.43 (s, 1H), 8.23 (d, J=8.8 Hz, 1H), 8.04 (d, J=8.4 Hz, 1H), 7.75 (d, J=8.4 Hz, 1H), 7.53-7.49 (m, 2H), 7.48-7.40 (m, 3H), 7 11 (dd, J=1.6, 8.8 Hz, 1H), 6.76-6.73 (m, 1H), 3.60 (s, 3H), 2.53-2.52 (m, 1H), 2.35 (s, 3H), 2.33-2.28 (m, 1H), 1.98-1.92 (m, 2H), 1.91-1.85 (m, 2H), 1.52-1.41 (m, 2H), 1.40-1.29 (m, 2H).

Step 4. To a solution of methyl (1r,4r)-4-((6-methyl-5-((4-nitro-[1,1′-biphenyl]-3-yl)amino)pyridin-2-yl)carbamoyl)cyclohexane-1-carboxylate (140 mg, 0.287 mmol) in THF (10 mL), wet Pd/C (50 mg, purity 10%) was added under N₂. The reaction mixture was degassed and purged with H₂ (15 psi) (3) and the mixture was stirred at 25° C. for 12 hrs under H₂ (15 psi). The reaction mixture was filtered and the filtrate was concentrated directly to give methyl (1r,4r)-4-((5-((4-amino-[1,1′-biphenyl]-3-yl)amino)-6-methylpyridin-2-yl)carbamoyl)cyclohexane-1-carboxylate (100 mg, 76.1% yield) as a light yellow oil, which was used in the next step directly. LCMS Rₜ=0.610 min in 1 min chromatography, purity 89.4%, MS ESI calcd. for 458.23, [M+H]⁺ 459.23, found 459.2.

Step 5. To a solution of methyl (1r,4r)-4-((5-((4-amino-[1,1′-biphenyl]-3-yl)amino)-6-methylpyridin-2-yl)carbamoyl)cyclohexane-1-carboxylate (100 mg, 0 218 mmol) in acetic acid (3 mL), 2-aminonicotinaldehyde (32.0 mg, 0.262 mmol) was added under O₂. The reaction mixture was degassed and purged with 02 (×3) and the mixture was stirred at 80° C. for 1 hr under 02. The reaction mixture was concentrated directly to give methyl (1r,4r)-4-((5-(2-(2-aminopyridin-3-yl)-6-phenyl-1H-benzo[d]imidazol-1-yl)-6-methylpyridin-2-yl)carbamoyl)cyclohexane-1-carboxylate (100 mg, 78.5% yield) as a light yellow oil, which was used in the next step directly. LCMS Rₜ=0.504 min in 1 min chromatography, purity 96.1%, MS ESI calcd. for 560.25 [M+H]⁺ 561.25, found 561.3.

Step 6. To a solution of methyl (1r,4r)-4-((5-(2-(2-aminopyridin-3-yl)-6-phenyl-1H-benzo[d]imidazol-1-yl)-6-methylpyridin-2-yl)carbamoyl)cyclohexane-1-carboxylate (100 mg, 0.178 mmol) in THF (3 mL) and water (3 mL), LiOH·H₂O (15.0 mg, 0.357 mmol) was added at 25° C. and the mixture was stirred for 12 hrs. The mixture was filtered directly. The residue was purified by prep-HPLC (Column: Welch Xtimate C18 150*30 mm*5 μm; mobile phase: [water (NH₄HCO₃)-ACN]; B %: 6%-46%; 25 min) to afford (1r,4r)-4-((5-(2-(2-aminopyridin-3-yl)-6-phenyl-1H-benzo[d]imidazol-1-yl)-6-methyl pyridin-2-yl)carbamoyl)cyclohexane-1-carboxylic acid 1-127 (88.1 mg, 90.3% yield) as a light yellow solid. ¹H NMR (DMSO-d₆, 400 MHz) δ_{H}=10.69 (s, 1H), 8.17 (d, J=8.8 Hz, 1H), 8.06-7.96 (m, 2H), 7.90 (d, J=8.4 Hz, 1H), 7.66 (d, J=7.6 Hz, 3H), 7.44-7.41 (m, 2H), 7.35-7.13 (m, 5H), 6.45-6.42 (m, 1H), 2.55-2.54 (m, 1H), 2.23-2.14 (m, 1H), 1.97-1.95 (m, 2H), 1.94-1.84 (m, 5H), 1.53-1.41 (m, 2H), 1.38-1.27 (m, 2H). HPLC Rₜ=2.748 min in 8 min chromatography, purity 99.9%. LCMS Rₜ=1.273 min in 2 min chromatography, purity 99.7%, MS ESI calcd. for 546.24 [M+H]⁺ 547.24, found 547 2.

### Example 119. Synthesis of 4-((5-(2-(2-aminopyridin-3-yl)-5-phenyl-3H-imidazo[4,5-b]pyridin-3-yl)-6-methylpyridin-2-yl)carbamoyl)-1-methylcyclohexane-1-carboxylic acid (Compound I-128)

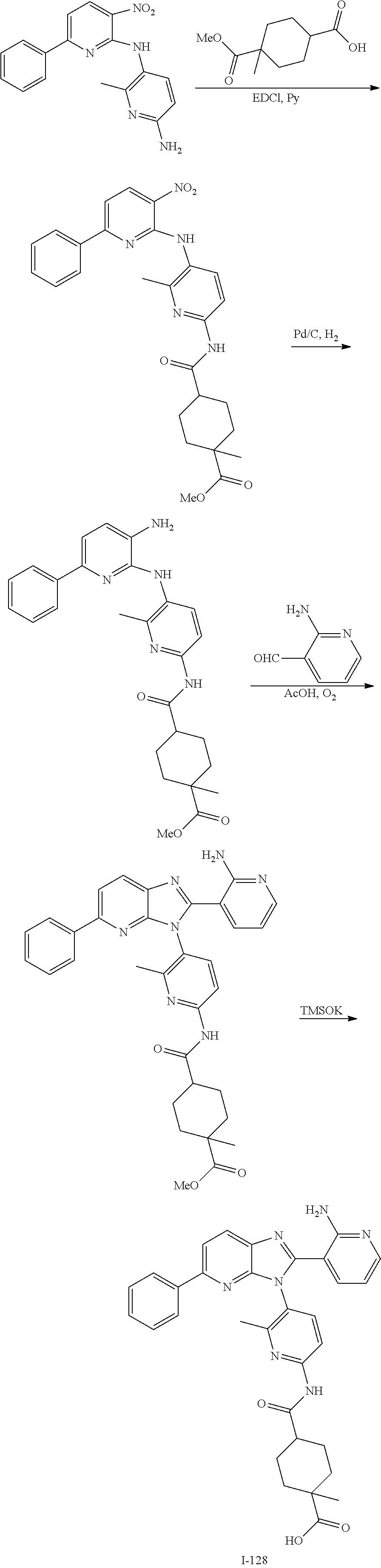

Step 1. To a solution of 6-methyl-N5-(3-nitro-6-phenylpyridin-2-yl)pyridine-2,5-diamine (500 mg, 1.56 mmol) and 4-methoxycarbonyl-4-methyl-cyclohexanecarboxylic acid (296 mg, 1.48 mmol) in pyridine (3 mL), EDCI (447 mg, 2.33 mmol) was added and the mixture was stirred at 80° C. for 6 hrs. Water (10 mL) was added to the residue and the resulting mixture was extracted with EtOAc (20 mL×3). The combined organic phase was washed with brine (10 mL), dried over anhydrous Na₂SO₄, filtered and concentrated. The residue was purified by flash silica gel chromatography (eluent of 0-33% EtOAc in PE) to give methyl 1-methyl-4-((6-methyl-5-((3-nitro-6-phenylpyridin-2-yl)amino)pyridin-2-yl)carbamoyl)cyclohexane-1-carboxylate (703 mg, 87.6% yield) as a red oil ¹H NMR (DMSO-d₆, 400 MHz) δ_{H}=10.47-10.42 (m, 1H), 9.96-9.91 (m, 1H), 8.64-8.59 (m, 1H), 8.03-7.91 (m, 4H), 7.59-7.53 (m, 1H), 7.52-7.46 (m, 3H), 3.67-3.64 (m, 3H), 2.39 (s, 4H), 2.19-2.12 (m, 2H), 1.79-1.71 (m, 2H), 1.45-1.38 (m, 2H), 1.24-1.20 (m, 2H), 1.15-1.12 (m, 3H).

Step 2. To a solution of methyl 1-methyl-4-((6-methyl-5-((3-nitro-6-phenylpyridin-2-yl)amino)pyridin-2-yl)carbamoyl)cyclohexane-1-carboxylate (300 mg, 0.596 mmol) in methanol (3 mL) and THF (3 mL), Pd/C (200 mg, wet, 10% purity) was added under Ar atmosphere. The suspension was degassed and purged with H₂ (×3) and the reaction mixture was stirred under H₂ (15 psi) at 25° C. for 12 hrs. The mixture was filtered through celite and washed with MeOH:DCM (1:5, 10 mL×4). The filtrate was concentrated to give methyl 4-((5-((3-amino-6-phenylpyridin-2-yl)amino)-6-methylpyridin-2-yl)carbamoyl)-1-methylcyclohexane-1-carboxylate (380 mg, crude) as a yellow solid. LCMS Rₜ=0.586 min in 2 min chromatography, purity 92.5%, MS ESI calcd for 473.24 [M+H]⁺ 474.24, found 474.1.

Step 3. To a solution of methyl 4-((5-((3-amino-6-phenylpyridin-2-yl)amino)-6-methylpyridin-2-yl)carbamoyl)-1-methylcyclohexane-1-carboxylate (80.0 mg, 0.802 mmol) in acetic acid (3 mL), 2-aminopyridine-3-carbaldehyde (147 mg, 1.20 mmol) was added. The reaction mixture was degassed and purged with O₂ (×3) and the mixture was stirred at 80° C. for 1 hr The residue was purified by flash silica gel chromatography (eluent of 0%-44% EtOAc in PE) to give methyl 4-((5-(2-(2-aminopyridin-3-yl)-5-phenyl-3H-imidazo[4,5-b]pyridin-3-yl)-6-methylpyridin-2-yl)carbamoyl)-1-methylcyclohexane-1-carboxylate (241 mg, 45.9% yield) as a yellow solid. ¹H NMR (DMSO-d₆, 400 MHz) δ1=10.68 (s, 1H), 8.30 (d, J=8.4 Hz, 1H), 8.09 (d, J=8.8 Hz, 1H), 8.04-7 97 (m, 4H), 7.86 (d, J=8.8 Hz, 1H), 7.49-7.36 (m, 3H), 7.23-7 11 (m, 3H), 6.46 (dd, J=4.8, 7.6 Hz, 1H), 3.65 (s, 3H), 3.31 (s, 4H), 2.15 (d, J=12.4 Hz, 2H), 1.80-1.72 (m, 2H), 1 47-1.36 (m, 2H), 1.25-1.18 (m, 2H), 1.13 (s, 3H).

Step 4. To a solution of methyl 4-((5-(2-(2-aminopyridin-3-yl)-5-phenyl-3H-imidazo[4,5-b]pyridin-3-yl)-6-methylpyridin-2-yl)carbamoyl)-1-methylcyclohexane-1-carboxylate (60.0 mg, 0.104 mmol) in ACN (3 mL), stirred with Al₂O₃ (53.0 mg, 0.521 mmol), TMSOK (67.0 mg, 0.521 mmol) was added and the reaction mixture was stirred at 40° C. for 12 hrs. The reaction mixture was concentrated directly. The crude product was purified by prep-HPLC (Column: Welch Xtimate C18 40*200 mm 7 μm; mobile phase: [water (FA)-ACN]; B %: 14%-54%; 25 min) to give 4-((5-(2-(2-aminopyridin-3-yl)-5-phenyl-3H-imidazo[4,5-b]pyridin-3-yl)-6-methylpyridin-2-yl)carbamoyl)-1-methylcyclohexane-1-carboxylic acid I-128 (18.5 mg, 31.6% yield) as a white solid. ¹H NMR (DMSO-d₆, 400 MHz) β_{H}=10.70 (s, 1H), 8.31 (d, J=8.4 Hz, 1H), 8.11 (d, J=8.6 Hz, 1H), 8.05-7.98 (m, 4H), 7.87 (d, J=8.8 Hz, 1H), 7.50-7.44 (m, 2H), 7.43-7.37 (m, 1H), 7.24-7.14 (m, 3H), 6.47 (dd, J=4.8, 7.6 Hz, 1H), 2.53-2.53 (m, 114), 2.13 (d, J=12.8 Hz, 2H), 2.07 (s, 3H), 1.75 (d, J=12.8 Hz, 2H), 1.55-1.42 (m, 2H), 1.20-1.10 (m, 5H). HPLC Rₜ=2.792 min in 8 min chromatography, purity 99.8%. LCMS Rₜ=1.315 min in 2 min chromatography, purity 99.7%, MS ESI calcd. for 561.25 [M+H]⁺ 562.25, found 562.2.

### Example 120. Synthesis of 3-((5-(2-(2-aminopyridin-3-yl)-5-phenyl-3H-imidazo[4,5-b]pyridin-3-yl)-6-methylpyridin-2-yl)carbamoyl)cyclopentane-1-carboxylic acid (Compound I-129)

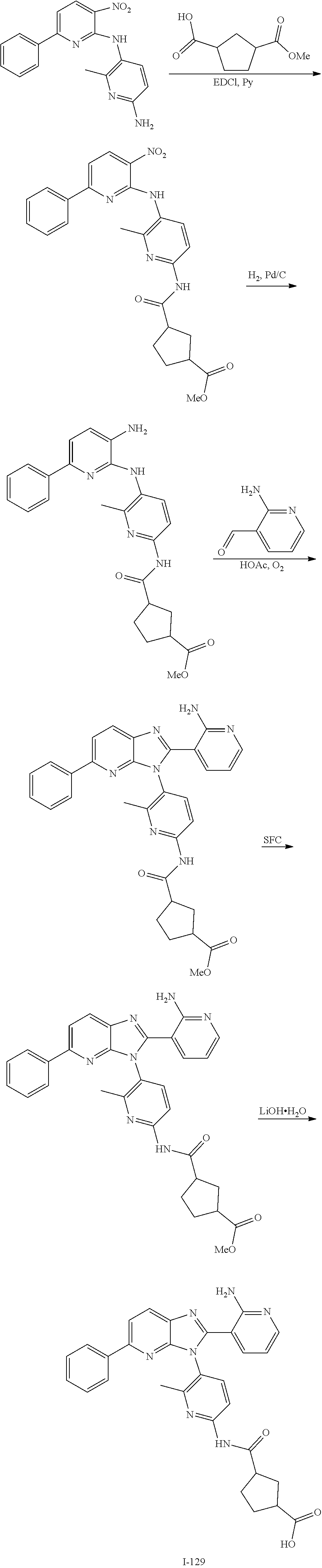

Step 1. To a solution of 6-methyl-N5-(3-nitro-6-phenylpyridin-2-yl)pyridine-2,5-diamine (1.00 g, 3.11 mmol) and 3-mnethoxycarbonylcyclopentanecarboxylic acid (589 mg, 3.42 mmol) in pyridine (5 mL, EDCI (895 mg, 4.67 mmol) was added and the mixture was stirred at 80′° C. for 1 hr. The reaction mixture was concentrated directly. Water (20 mL) was added and the resulting mixture was extracted with EtOAc (15 mL×3). The combined organic phase was washed with brine (15 mL), dried over anhydrous Na₂SO₄, filtered and concentrated. The residue was purified by flash silica gel chromatography (eluent of 0%-35% EtOAc in PE) to give methyl 3-((6-methyl-5-((3-nitro-6-phenylpyridin-2-yl)amino)pyridin-2-yl)carbamoyl)cyclopentane-1-carboxylate (1.08 g, 69.7% yield) as a yellow oil. ¹H NMR (DMSO-d₆, 400 MHz) δ_{H}=10.51 (s, 1H), 9.94 (s, 1H), 8.60 (d, J=8.8 Hz, 1H), 8.02 (q, J=8.8 Hz, 2H), 7.96-7.91 (m, 2H), 7.55 (d, 1=8.4 Hz, 1H), 7.51-7.46 (m, 3H), 3.62 (s, 3H), 3.07-2.98 (m, 1H), 2.90-2.79 (m, 1H), 2.39 (s, 3H), 2 23-2.14 (m, 1H), 2.01-1.93 (m, 1H), 1.91-1.78 (m, 4H).

Step 2. To a solution of methyl 3-((6-methyl-5-((3-nitro-6-phenylpyridin-2-yl)amino)pyridin-2-yl)carbamoyl)cyclopentane-1-carboxylate (1.08 g, 2.27 mmol) in THF (15.0 mL), wet Pd/C (0.60 g, wet, 10% purity) was added under Ar. The reaction mixture was degassed and purged with H₂ (×3) and the mixture was stirred at 25° C. for 12 hrs under H₂ (15 psi). The mixture was filtered through celite and washed with MeOH in DCM (20%, 10 mL×4). The filtrate was concentrated to give methyl 3-((5-((3-amino-6-phenylpyridin-2-yl)amino)-6-methylpyridin-2-yl)carbamoyl)cyclopentane-1-carboxylate (976 mg, 95.9% yield) as a yellow solid. ¹H NMR (DMSO-d₆, 400 MHz) δ_{H}=10.36 (s, 1H), 7.97-7.86 (m, 2H), 7.75 (d, J=7.2 Hz, 2H), 7.34-7.26 (m, 3H), 7.23-7.16 (m, 2H), 6.96 (d, J=8.0 Hz, 1H), 5.21 (s, 2H), 3.61 (s, 3H), 3.00 (t, J=8.4 Hz, 1H), 2.90-2.78 (m, 1H), 2.36 (s, 3H), 2.21-2.12 (m, 1H), 1.98-1.90 (m, 1H), 1.89-1.74 (m, 4H).

Step 3. To a solution of methyl 3-((5-((3-amino-6-phenylpyridin-2-yl)amino)-6-methylpyridin-2-yl)carbamoyl)cyclopentane-1-carboxylate (500 mg, 1.12 mmol) in acetic acid (3 mL), 2-aminopyridine-3-carbaldehyde (206 mg, 1.68 mmol) under N₂. The reaction mixture was degassed and purged with O₂ (×3) and the mixture was stirred at 80° C. for 1 hr. The reaction mixture was concentrated. The residue was purified by flash silica gel chromatography (eluent of 0%-39% of EtOAc in PE) to give methyl 3-((5-(2-(2-aminopyridin-3-yl)-5-phenyl-3H-imidazo[4,5-b]pyridine-3-yl)-6-methylpyridin-2-yl) carbamoyl) cyclopentane-1-carboxylate (336 mg, 52.4% yield) as a yellow solid. ¹H NMR (DMSO-d₆, 400 MHz) δ_{H}=10.75 (s, 1H), 8.30 (d, J=8.4 Hz, 1H), 8.12 (d, J=8.8 Hz, 1H), 8.05-7.96 (m, 4H), 7.87 (d, J1=88 Hz, 1H), 7.49-7.36 (m, 3H), 7.23-7.12 (m, 3H), 6.46 (dd, J=4.8, 7.6 Hz, 1H), 3.61 (s, 3H), 3.07-3.01 (m, 1H), 2.89-2 83 (m, 1H), 2.23-2.14 (m, 1H), 2.06 (s, 3H), 2.02-1.95 (m, 1H), 1.94-1.81 (m, 4H).

Step 4. Methyl 3-((5-(2-(2-aminopyridin-3-yl)-5-phenyl-3H-imidazo[4,5-b]pyridine-3-yl)-6-methylpyridin-2-yl) carbamoyl) cyclopentane-1-carboxylate was purified by SFC (column: DAICEL CHIRALPAK AD (250 mm*30 mm, 10 um)); mobile phase:[CO₂-i-PrOH]; B %: 55%-55%, 6 min) to give methyl 3-((5-(2-(2-aminopyridin-3-yl)-5-phenyl-3H-imidazo[4,5-b]pyridin-3-yl)-6-methylpyridin-2-yl)carbamoyl)cyclopentane-1-carboxylate (187 mg, 55.7% yield) as a yellow solid, which remained the mixture of diastereomers. ¹H NMR (400 MHz, DMSO-d₆) δ=10.80 (s, 1H), 8.35 (d, J=8.4 Hz, 1H), 8.17 (d, J=8.4 Hz, 1H), 8.09-8.02 (m, 4H), 7.92 (d, J=8.8 Hz, 1H), 7.54-7.40 (m, 3H), 7.27-7.18 (m, 3H), 6.51 (dd, J=4.8, 7.6 Hz, 1H), 3.66 (s, 3H), 3.11-3.07 (m. IH), 2.91-2.84 (m, 1H), 2.11 (s, 3H), 2.05-1.89 (m, 6H).

Step 5. To a solution of methyl 3-((5-(2-(2-aminopyridin-3-yl)-5-phenyl-3H-imidazo[4,5-b]pyridin-3-yl)-6-methylpyridin-2-yl)carbamoyl)cyclopentane-1-carboxylate (44.0 mg, 0.0803 mmol) in THF (2 mL), water (1 mL) and LiOH·H₂O (17.0 mg, 0.402 mmol) were added and the mixture was stirred at 40° C. for 12 hrs. The reaction mixture was concentrated directly. The residue was purified by prep-HPLC (Column: Welch Xtimate C18 40*200 mm 7 μm; mobile phase: [water (FA)-ACN]; B %: 6%-46%; 20.5 min) to provide 3-((5-(2-(2-aminopyridin-3-yl)-5-phenyl-3H-imidazo[4,5-b]pyridin-3-yl)-6-methylpyridin-2-yl)carbamoyl)cyclopentane-1-carboxylic acid 1-129 (10.8 mg, 25.2% yield) as a white solid. ¹H NMR (DMSO-d₆, 400 MHz) δ_{H}=10.88-10.70 (m, 1H), 8.30 (d, J=8.4 Hz, 1H), 8.13 (d, J=8.8 Hz, 1H), 8.05-7.98 (m, 4H), 7.87 (d, J=8.4 Hz, 1H), 7.50-7.36 (m, 3H), 7.23-7.14 (m, 3H), 6.47 (dd, J=4.8, 7.6 Hz, 1H), 3.07-2.96 (m, 1H), 2.73 (t, J=8.4 Hz, 1H), 2.20-2.10 (m, 1H), 2.07 (s, 3H), 2.05-1.91 (m, 2H), 1.89-1.72 (m, 3H). ¹H NMR (400 MHz, DMSO-d₆, t=80) δ=10.43 (s, 1H), 8.27 (d, J=8.4 Hz, 1H), 8.11 (d, J=8.8 Hz, 1H), 8.03 (dd, J=1.6, 4.8 Hz, 1H), 8.01-7.94 (m, 3H), 7.84 (d, J=8.4 Hz, 1H), 7.49-7.34 (m, 3H), 7.21 (dd, J=1.2, 7.6 Hz, 1H), 6.99 (s, 2H), 6.46 (dd, J=4.8, 7.6 Hz, 1H), 2.78-2.73 (m, 1H), 2.27-2.15 (m, 1H), 2.12-1.98 (m, 6H), 1.94-1.84 (m, 3H). HPLC Rₜ=2.432 min in 8.0 min chromatography, purity 99.8%. LCMS Rₜ=1.164 min in 2 min chromatography, purity 99.1%, MS ESI calcd. for 533.22 [M+H]⁺ 534.22, found 534 2.

### Example 121. Synthesis of (1r,4r)-4-((5-(2-(2-aminopyridin-3-yl)-6-(pyridin-2-yl)-1H-benzo[d]imidazol-1-yl)-6-methylpyridin-2-yl)carbamoyl)cyclohexane-1-carboxylic acid (Compound I-130)

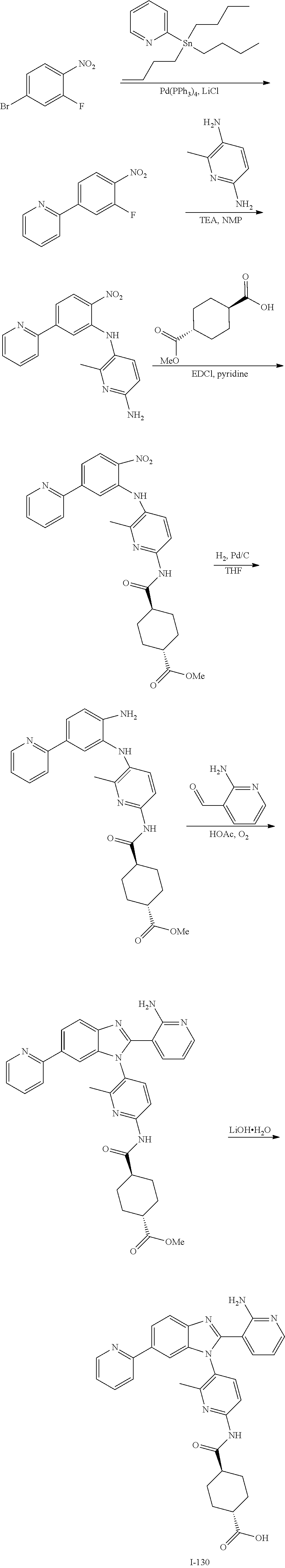

Step 1. To a solution of tributyl (2-pyridyl) stannane (18.8 g, 51.1 mmol), 4-bromo-2-fluoro-1-nitro-benzene (4.50 g, 20.5 mmol) in 1,4-dioxane (100 mL), LiCl (2.60 g, 61.4 mmol) and Pd(PPh₃)₄ (473 mg, 0.409 mmol) were added under N₂ atmosphere and the mixture was stirred at 100° C. for 6 hrs. The residue was dissolved in sat. aq. KF (100 mL). The resulting suspension was extracted with EtOAc (100 mL×3). The combined organic phase was washed with brine (100 mL), water (100 mL), dried over anhydrous Na₂SO₄, filtered and concentrated. The residue was purified by Combi-Flash column (EtOAc in PE, M/%-15%) to give 2-(3-fluoro-4-nitro-phenyl)pyridine (2.40 g, 53.8% yield) as a red solid. LCMS Rₜ=3.015 min in 7 min chromatography, MS ESI calcd. for 218.05 [M+H]⁺ 219.05, found 219.0.

Step 2. To a solution of 6-methylpyridine-2,5-diamine (677 mg, 5.50 mmol) in NMP (5 mL), TEA (2.30 mL, 16.5 mmol) and 2-(3-fluoro-4-nitro-phenyl)pyridine (1.20 g, 5.50 mmol) were added under N₂ atmosphere and the mixture was subjected to microwave irradiation at 160° C. for 2 hrs. The reaction mixture was filtered and the filtrate was concentrated. The residue was dissolved in water (20 mL) and the resulting suspension was extracted with EtOAc (20 mL×3). The combined organic phase was washed with brine (50 mL), water (50 mL), dried over anhydrous Na₂SO₄, filtered and concentrated. The residue was purified by Combi-Flash column (EtOAc in PE, 0%-7%) to give 6-methyl-N5-[2-nitro-5-(2-pyridyl)phenyl]pyridine-2,5-diamine (450 mg, 25.5% yield) as a red solid. ¹H NMR (DMSO-d₆, 400 MHz) δ_{H}=9.26 (s, 1H), 8.64 (d, J=4.4 Hz, 1H), 8.22 (d, J=9.2 Hz, 1H), 7.96-7.80 (m, 2H), 7.45-7.23 (m, 4H), 6.39 (d, J=8.4 Hz, 1H), 6.03 (s, 2H), 2.15 (s, 3H).

Step 3. A mixture of 6-methyl-N5-[2-nitro-5-(2-pyridyl)phenyl]pyridine-2,5-diamine (450 mg, 1.40 mmol), trans-4-methoxycarbonylcyclohexanecarboxylic acid (313 mg, 1.68 mmol) and EDCI (322 mg, 1.68 mmol) in pyridine (5 mL) was degassed and purged with N₂ (×3), and the mixture was stirred at 40° C. for 12 hrs under N₂ atmosphere. The reaction mixture was filtered and the filtrate was concentrated. The residue was dissolved in water (50 mL) and the resulting suspension was extracted with EtOAc (20 mL×3). The combined organic phase was washed with brine (50 mL), water (50 mL), dried over anhydrous Na₂SO₄, filtered and concentrated. The residue was purified by Combi-Flash column (EtOAc in PE=50%-80%) to give trans-methyl 4-[[6-methyl-5-[2-nitro-5-(2-pyridyl)anilino]-2-pyridyl]carbamoyl]cyclohexanecarboxylate (370 mg, 54.0% yield) as a red solid. ¹H NMR (DMSO-d₆, 400 MHz) δ_{H}=10.50 (s, 1H), 9.41 (s, 1H), 8.62 (d, =4.4 Hz, 1H), 8.25 (d, J=9.2 Hz, 1H), 8.04 (d, J=8.4 Hz, 1H), 7.94-7.86 (m, 2H), 7.73 (d, J=8.4 Hz, 1H), 7.47 (dd, J=1.6, 8.8 Hz, 1H), 7.43-7.35 (m, 2H), 3.60 (s, 3H), 2.37-2.28 (m, 4H), 1.99-1.87 (m, 4H), 1.52-1.31 (m, 4H).

Step 4. To a solution of trans-methyl 4-[[6-methyl-5-[2-nitro-5-(2-pyridyl)anilino]-2-pyridyl]carbamoyl]cyclohexanecarboxylate (487 mg, 0.995 mmol) in THF (5 mL), wet Pd/C (0.1 g, purity 10%) was added under N₂ atmosphere. The suspension was degassed and purged with H₂ (×3) and the reaction mixture was stirred under H₂ (15 psi) at 25° C. for 16 hrs The reaction mixture was filtered via a celite pad, and the filter cake was washed with DCM:MeOH (10:1, 100 mL). The filtrate was concentrated to give trans-methyl 4-[[5-[2-amino-5-(2-pyridyl)anilino]-6-methyl-2-pyridyl]carbamoyl]cyclohexanecarboxylate (380 mg, 83.1% yield) as a yellow solid. ¹H NMR (400 MHz, DMSO-d₆) δ=10.14 (s, 1H), 8.54 (d, J=4.4 Hz, 1H), 7.84-7.72 (m, 3H), 7.62-7.56 (m, 2H), 7.19 (ddd, J=1.6, 4.8, 6.4 Hz, 1 HI), 7.02 (d, J=8.8 Hz, 1H), 6.85 (d, J=8.0 Hz, 1H), 6.63 (s, 1H), 3.65 (s, 3H), 2.43 (s, 3H), 2.03-1.97 (m, 2H), 1.93-1.87 (m, 2H), 1.56-1.31 (m, 6H).

Step 5. To a solution of trans-methyl 4-[[5-[2-amino-5-(2-pyridyl)anilino]-6-methyl-2-pyridyl]carbamoyl]cyclohexanecarboxylate (330 mg, 0.718 mmol) in acetic acid (5 mL), 2-aminopyridine-3-carbaldehyde (132 mg, 1.08 mmol) was added The reaction mixture was degassed and purged with 02 (×3) and the mixture was stirred at 80° C. for 2 hrs. The reaction solution was concentrated directly to give trans-methyl 4-[[5-[2-(2-amino-3-pyridyl)-6-(2-pyridyl) benzimidazol-1-yl]-6-methyl-2-pyridyl]carbamoyl]cyclohexanecarboxylate (280 mg, crude) as yellow oil. LCMS Rₜ=0.525 min in 1 min chromatography, purity 86.9%, MS ESI calcd. for 561.25 [M+H]⁺ 562.25, found 562.2.

Step 6. To a solution of trans-methyl 4-[[5-[2-(2-amino-3-pyridyl)-6-(2-pyridyl) benzimidazol-1-yl]-6-methyl-2-pyridyl]carbamoyl]cyclohexanecarboxylate (280 mg, 0.499 mmol) in water (1 mL) and THE (4 mL), LiOH (34.0 mg, 1.50 mmol) was added and the mixture was stirred at 25° C. for 12 hrs. The residue was purified by prep-HPLC (Column: Xtimate C18 200*40 mm*7 μm; mobile phase: [water (NH₄HCO₃)-ACN]; B %: 2%-42%, 25 min) to give (1r,4r)-4-((5-(2-(2-aminopyridin-3-yl)-6-(pyridin-2-yl)-1H-benzo[d]imidazol-1-yl)-6-methylpyridin-2-yl)carbamoyl)cyclohexane-1-carboxylic acid 1-130 (183 mg, 67.1% yield) as a white solid. ¹H NMR (DMSO-d₆, 400 MHz) α_{H}=10.72 (s, 1H), 8.61 (d, J=4.4 Hz, 1H), 8.19 (d, J=8.4 Hz, 1H), 8.11 (dd, J=1.6, 8.4 Hz, 1H), 8.06-7.98 (m, 3H), 7.91 (d, J=8.4 Hz, 1H), 7.84 (dt, J=1.6, 7.6 Hz, 1H), 7.77 (d, J=1.2 Hz, 1H), 7.31 (dd, J=4.8, 7.2 Hz, 1H), 7.24-7.15 (m, 3H), 6.44 (dd, J=4.8, 7.6 Hz, 1H), 2.52 (s, 1H), 2.24-2.16 (m, 1H), 2.00-1.94 (m, 2H), 1.93-1.86 (m, 5H), 1.53-1.40 (m, 2H), 1.33 (q, J=12.4 Hz, 2H). HPLC Rₜ=4.088 min in 8 min chromatography, purity 98.4%. LCMS Rₜ=2.519 min in 4 min chromatography, purity 98.5%, MS ESI calcd. for 547.23 [M+H]⁺ 548.23, found 548.2.

### Example 122. Synthesis of (1r,4r)-4-(((5-(2-(2-aminopyridin-3-yl)-5-phenyl-3H-imidazo[4,5-b]pyridin-3-yl)-6-methylpyridin-2-yl)amino)methyl)cyclohexane-1-carboxylic acid (Compound I-131)

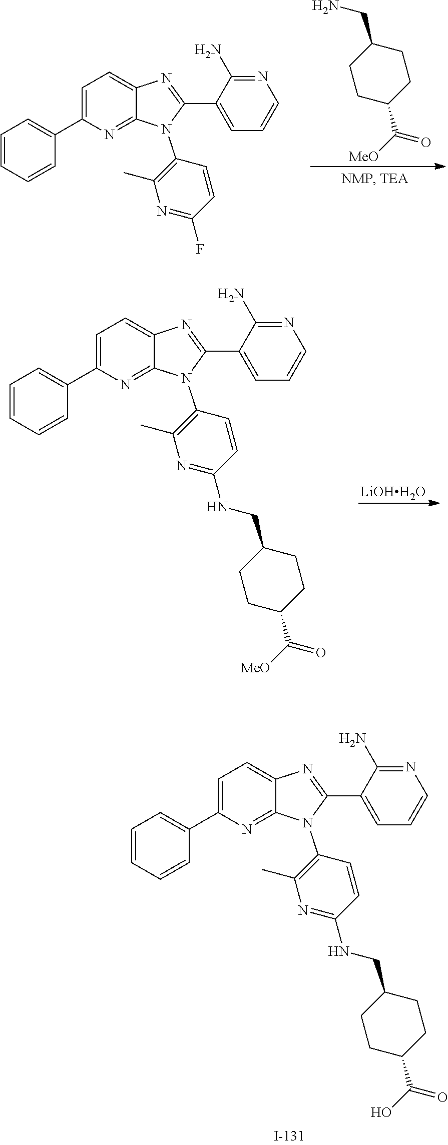 [SMILES: Cc1nc(F)ccc1-n1c(-c2cccnc2N)nc2ccc(-c3ccccc3)nc21 ; COC(=O)[C@H]1CC[C@H](CN)CC1 ; COC(=O)[C@H]1CC[C@H](CNc2ccc(-n3c(-c4cccnc4N)nc4ccc(-c5ccccc5)nc43)c(C)n2)CC1 ; "LiOH·H2O" ; Cc1nc(NC[C@H]2CC[C@H](C(=O)O)CC2)ccc1-n1c(-c2cccnc2N)nc2ccc(-c3ccccc3)nc21]

Step 1. To a solution of 3-(3-(6-fluoro-2-methylpyridin-3-yl)-5-phenyl-3H-imidazo[4,5-b]pyridin-2-yl)pyridin-2-amine (120 mg, 0.300 mmol) in NMP (2.00 mL), TEA (0.13 mL, 0.90 mmol) and methyl (1r,4r)-4-(aminomethyl)cyclohexane-1-carboxylate (125 mg, 0.605 mmol) were added under N₂ atmosphere and the mixture was subjected to microwave irradiation at 150° C. for 2 hrs. EtOAc (20 mL) was added to the mixture and the resulting suspension was washed with brine (20 mL×2), dried over anhydrous Na₂SO₄, filtered and concentrated. The residue was purified by Combi-Flash column (EtOAc in PE, 0%-65%) to give methyl (1r,4r)-4-(((5-(2-(2-aminopyridin-3-yl)-5-phenyl-3H-imidazo[4,5-b]pyridin-3-yl)-6-methylpyridin-2-yl)amino)methyl)cyclohexane-1-carboxylate (190 mg) as a yellow oil. ¹H NMR (DMSO-d₆, 400 MHz) δ_{H}=8.25 (d, J=8.4 Hz, 1H), 8.04-7.93 (m, 5H), 7.46 (d, J=7.6 Hz, 2H), 7.38 (d, J=9.2 Hz, 2H), 7.22 (s, 1H), 6.88-6.82 (m, 1H), 6.49-6.43 (m, 2H), 3.60-3.57 (m, 3H), 3.17-3.08 (m, 2H), 1.93-1.86 (m, 9H), 1.39-1.26 (m, 3H), 1.08-0.98 (m, 2H).

Step 2. To a solution of methyl (1r,4r)-4-(((5-(2-(2-aminopyridin-3-yl)-5-phenyl-3H-imidazo[4,5-b]pyridin-3-yl)-6-methylpyridin-2-yl)amino)methyl)cyclohexane-1-carboxylate (190 mg, 0.340 mmol) in THF (2 mL) and water (2 mL), LiOH·H₂O (73.0 mg, 1.73 mmol) was added at 25° C. and the mixture was stirred for 14 hrs. FA (2 mL) was added to the mixture. The residue was purified by prep-HPLC (condition: water (NH₄HCO₃)-ACN; Column: Welch Xtimate C18 40*200 mm*7 μm; B %: 8%-48%; Gradient Time, 25 min) to give (1r,4r)-4-(((5-(2-(2-aminopyridin-3-yl)-5-phenyl-3H-imidazo[4,5-b]pyridin-3-yl)-6-methylpyridin-2-yl)amino)methyl)cyclohexane-1-carboxylic acid 1-131 (43.9 mg, 23.4% yield) as a yellow solid. ¹H NMR (DMSO-d₆, 400 MHz) δ_{H}=8.26 (d, =8.4 Hz, 1H), 8.05-7.98 (m, 3H), 7.96 (d, J=8.4 Hz, 1H), 7.50-7.44 (m, 2H), 7.42-7.36 (m, 2H), 7.29-7.19 (m, 3H), 6.85 (t, J=4.8 Hz, 1H), 6.49-6.42 (m, 2H), 3.21-3.04 (m, 3H), 2.15 (t, J=12.4 Hz, 1H), 1.94 (d, J=2.0 Hz, 1H), 1.91-1.83 (m, 5H), 1.58-1.44 (m, 1H), 1.38-1.23 (m, 2H), 1.08-0.93 (m, 2H). HPLC Rₜ=2.041 min in 8 min chromatography, purity 98.8%. LCMS Rₜ=1.438 min in 4 min chromatography, purity 99.4%, MS ESI calcd. for 533.25 [M+H]⁺ 534.25, found 534.3.

### Example 123. Synthesis of 2-(3-((5-(2-(2-aminopyridin-3-yl)-5-phenyl-3H-imidazo[4,5-b]pyridin-3-yl)-6-methylpyridin-2-yl)carbamoyl)cyclopentyl)propanoic acid (Compound I-132)

### Synthesis of 3-(2-ethoxy-1-methyl-2-oxo-ethyl)cyclopentanecarboxylic acid

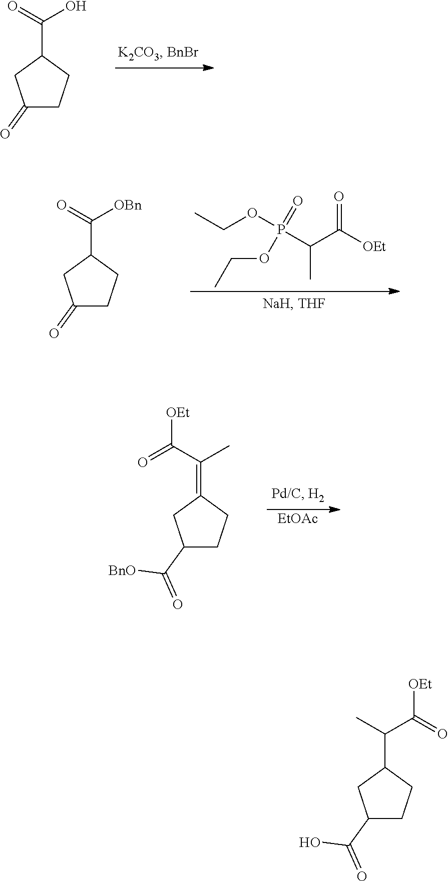 [SMILES: "EtOAc" ; O=C1CCC(C(=O)O)C1 ; O=C1CCC(C(=O)OCc2ccccc2)C1 ; CCOC(=O)C(C)P(=O)(OCC)OCC ; CCOC(=O)/C(C)=C1/CCC(C(=O)OCc2ccccc2)C1 ; CCOC(=O)C(C)C1CCC(C(=O)O)C1]

Step 1. To a solution of 3-oxocyclopentanecarboxylic acid (5.00 g, 39.0 mmol) in DMF (50.0 mL), K₂CO₃ (10.8 g, 78.0 mmol) and bromomethylbenzene (5.60 mL, 46.8 mmol) were added and the mixture was stirred at 25° C. for 16 hrs Water (50 mL) was added and the mixture was extracted with EtOAc (50 mL×3). The organic phase was washed with brine (10 mL×3), dried with Na₂SO₄ and concentrated. The mixture was purified by column (EtOAc in PE, 0-30%) to give benzyl 3-oxocyclopentanecarboxylate (7.70 g, 90.4% yield) as a colorless oil. 1H NMR (CHLOROFORM-d 400 MHz) δ_{H}=7.40-7.30 (m, 5H), 5.16 (s, 2H), 3.23-3.10 (m, 1H), 2.59-2.26 (m, 4H), 2.26-2.09 (m, 2H).

Step 2. To a solution of ethyl 2-diethoxyphosphorylpropanoate (18.0 g, 75.6 mmol) in THF (30 mL), NaH (3.02 g, 75.6 mmol) was added at 0° C., and the mixture was stirred at 20° C. for 1 hr. A solution of benzyl 3-oxocyclopentanecarboxylate (5.50 g, 25.2 mmol) in THF (10 mL) was added dropwise to the mixture and the mixture was stirred at 20° C. for 2 hrs. Water (50 mL) was added and the mixture was extracted with EtOAc (50 mL×2). The organic phase was washed with brine (20 mL), dried with Na₂SO₄ and concentrated. The residue was purified by column (0-10% of EtOAc in PE) to give crude product. The residue was purified by prep-HPLC (Welch Xtimate C18 150*30 mm*5 μm, water (FA)-ACN) to give benzyl (3Z)-3-(2-ethoxy-1-methyl-2-oxo-ethylidene) cyclopentanecarboxylate (500 mg, 6.56% yield) as a yellow oil. LCMS Rₜ=0.803 min in 4 min chromatography, 5-95AB_1min_220&254_Agilent.M, purity 97.9%, MS ESI calcd. for 302.2 [M+Na]⁺ 325.2, found 325.1.

Step 3. To a solution of benzyl (3Z)-3-(2-ethoxy-1-methyl-2-oxo-ethylidene) cyclopentanecarboxylate (500 mg, 1.65 mmol) in ethyl acetate (10 mL), wet Pd/C (100 mg, purity 10%) was added under argon. The reaction was degassed and purged with Ar (×3), then degassed and purged with H₂ (×3) and stirred under H₂ (50 psi) for 16 hrs at 50° C. Upon completion, the mixture was filtered under argon to afford 3-(2-ethoxy-1-methyl-2-oxo-ethyl)cyclopentanecarboxylic acid (370 mg, crude) as colorless oil. ¹H NMR (CHLOROFORM-d₄ 400 MHz) δ_{H}=4.19-4.05 (m, 2H), 2.94-2.71 (m, 1H), 2.43-2.19 (m, 1H), 2.17-1.99 (m, 2H), 1.96-1.71 (m, 3H), 1.64-1.30 (m, 2H), 1.28-1.22 (m, 3H), 1.22-1.14 (m, 3H).

### Synthesis of 2-(3-((S-(2-(2-aminopyridin-3-yl)-5-phenyl-3H-imidazo[4,5-b]pyridin-3-yl)-6-methylpyridin-2-yl)carbamoyl)cyclopentyl)propanoic acid (Compound I-132)

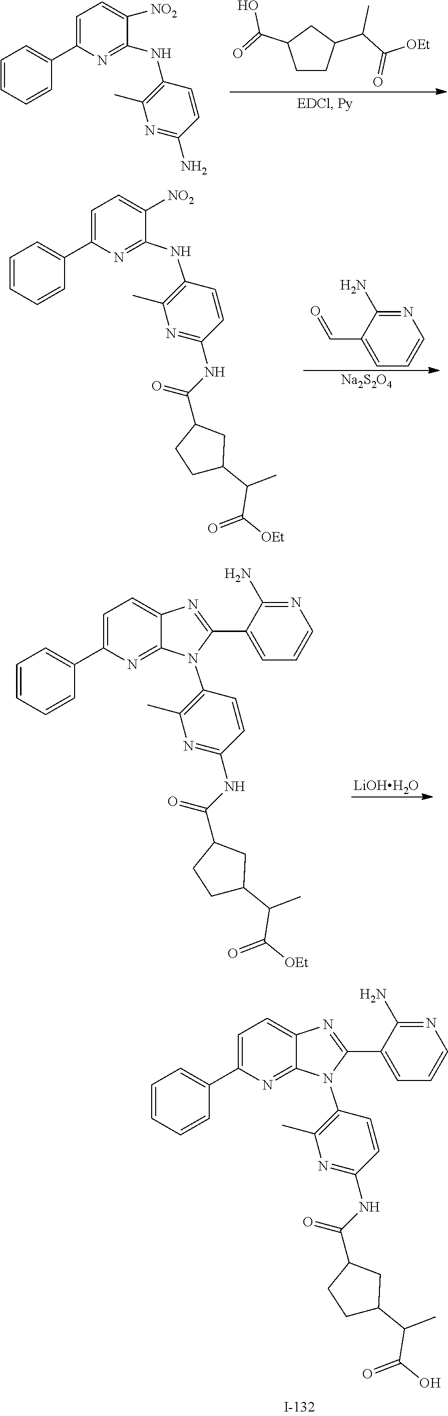

Step 1. To a solution of 6-methyl-N5-(3-nitro-6-phenyl-2-pyridyl)pyridine-2,5-diamine (600 mg, 1.87 mmol) and 3-(2-ethoxy-1-methyl-2-oxo-ethyl)cyclopentanecarboxylic acid (374 mg, 1.87 mmol) in pyridine (18 mL), EDCI (796 mg, 5.60 mmol) was added and the mixture was stirred at 80° C. for 16 hrs. Water (20 mL) was added to the mixture, and the aqueous phase was extracted with EtOAc (20 mL×3). The organic phase was washed with 1M HCl (10 mL), brine (30 mL), dried with Na₂SO₄ and concentrated. The residue was purified by column (EtOAc in PE, 0-20%) to give ethyl 2-(3-((6-methyl-5-((3-nitro-6-phenylpyridin-2-yl)amino)pyridin-2-yl)carbamoyl)cyclopentyl)propanoate (400 mg, 42.5% yield) as a yellow solid. LCMS Rₜ=0.792 min in 1 min chromatography, 5-95AB_1 min_220&254_Agilent.M, purity 100% N, MS ESI calcd. for 517.2 [M+H]⁺ 518.2, found 518.2.

Step 2. To a solution of ethyl 2-(3-((6-methyl-5-((3-nitro-6-phenylpyridin-2-yl)amino)pyridin-2-yl)carbamoyl)cyclopentyl)propanoate (500 mg, 0.966 mmol) and 2-aminopyridine-3-carbaldehyde (159 mg, 1.30 mmol) in DMSO (5.00 mL) and methanol (0.5 mL), Na₂S₂O₄ (505 mg, 2.90 mmol) was added under N₂ and the mixture was stirred at 90° C. for 12 hrs. Water (20 mL) was added to the mixture, and the whole extracted with EtOAc (30 mL×3). The combined organic phase was washed with brine (30 mL), dried over anhydrous Na₂SO₄, filtered and concentrated The mixture was purified by prep-HPLC (Welch Xtimate C18 150*30 mm*5 μm, water(HCl)-ACN) to give ethyl 2-[3-[[5-[2-(2-amino-3-pyridyl)-5-phenyl-imidazo[4,5-b]pyridin-3-yl]-6-methyl-2-pyridyl]carbamoyl]cyclopentyl]propanoate (50.0 mg, 8.78% yield) as a yellow solid. LCMS Rₜ=2 917 min in 4 min chromatography, 10-80AB_4 min_220&254_Shimadzu.1 cm, purity 93.2%, MS ESI calcd. for 589.3 [M+H]⁺ 590.3, found 590.1.

Step 3. To a solution of ethyl 2-[3-[[5-[2-(2-amino-3-pyridyl)-5-phenyl-imidazo[4,5-b]pyridin-3-yl]-6-methyl-2-pyridyl]carbamoyl]cyclopentyl]propanoate (50.0 mg, 0.0848 mmol) in THF (1 mL) and water (1 mL), LiOH·H₂O (11.0 mg, 0.254 mmol) was added and the mixture was stirred at 30° C. for 16 hrs. To the mixture LiOH·H₂O (11.0 mg, 0.254 mmol) was added and the mixture was stirred at 35° C. for 4 hrs. The mixture was adjusted to pH ~7 and concentrated. The residue was purified by prep-HPLC (Welch Xtimate C18 150*30 mm*5 μm, water (FA)-ACN) to give 2-[3-[[5-[2-(2-amino-3-pyridyl)-5-phenyl-imidazo[4,5-b]pyridin-3-yl]-6-methyl-2-pyridyl]carbamoyl]cyclopentyl]propanoic acid I-132 (27.0 mg, 55.9% yield) as a yellow solid. ¹H NMR (DMSO-d₆, 400 MHz) δ_{H}=12.71-11.37 (m, 1H), 10.86-10 59 (m, 1H), 8.33 (d, J=8.4 Hz, 1H), 8.14 (d, J=8.8 Hz, 1H), 8.08-7.95 (m, 4H), 7.92-7.83 (m, 1H), 7.58-7.45 (m, 3H), 7.44-7.31 (m, 2H), 6.65-6.53 (m, 1H), 2.82-2.65 (m, 1H), 2.18-2.10 (m, 3H), 2.08-2.00 (m, 1H), 1 98-1 88 (m, 1H), 1.84-1.73 (m, 2H), 1 72-1.61 (m, 1H), 1.60-1.41 (m, 1H), 1.39-1.17 (m, 2H), 1.12 (dd, J=3.2, 6.8 Hz, 3H). HPLC Rₜ=4.458 min, 4.504 min, 4.630 min, 4.749 min and 4.785 min in 8 min chromatography, 0-60AB_8 min.1 cm, purity 100%. LCMS Rₜ=3.097 min, 3.187 min and 3.289 min in 4 min chromatography, 0-60AB_4 min_220&254_Shimadzu.1 cm, purity 100%, MS ESI calcd. for 561 3 [M+H]⁺562.3, found 562.1.

### Example 124. Synthesis of (1r,4r)-4-((6-(2-(2-aminopyridin-3-yl)-6-(pyridin-2-yl)-1H-benzo[d]imidazol-1-yl)-2-methylpyridin-3-yl)carbamoyl)cyclohexane-1-carboxylic acid (Compound I-133)

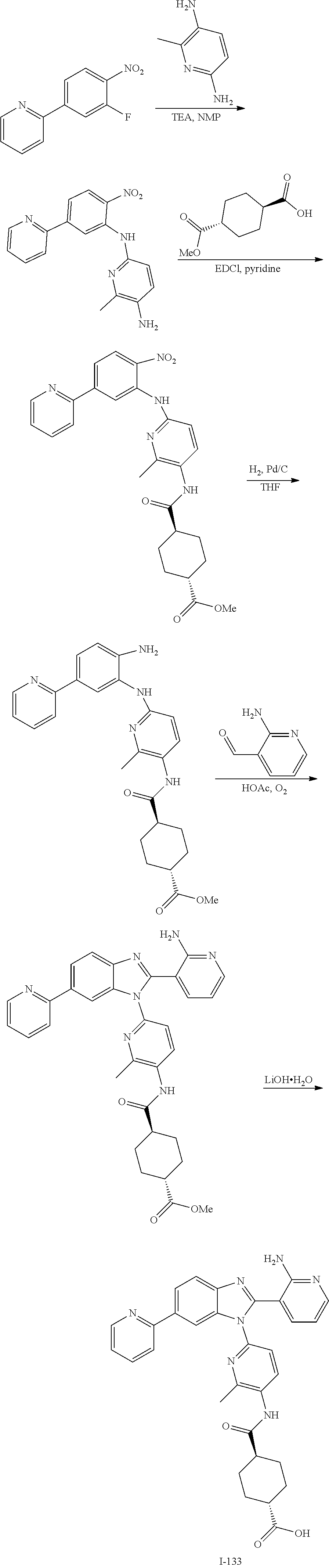

Step 1. To a solution of 6-methylpyridine-2,5-diamine (677 mg, 5.50 mmol) in NMP (5 mL), TEA (2.30 mL, 16.5 mmol) and 2-(3-fluoro-4-nitro-phenyl)pyridine (1.20 g, 5.50 mmol) were added under N₂ atmosphere and the mixture was subjected to microwave irradiation at 160° C. for 2 hrs. The reaction mixture was filtered and the filtrate was concentrated. The residue was dissolved in water (20 mL) and the resulting suspension was extracted with EtOAc (20 mL×3). The combined organic phase was washed with brine (50 mL), water (50 mL), dried over anhydrous Na₂SO₄, filtered and concentrated. The residue was purified by Combi-Flash column (EtOAc in PE, 0%-7%) to give 6-methyl-N2-(2-nitro-5-(pyridin-2-yl)phenyl)pyridine-2,5-diamine (450 mg, 25.5% yield) as a red solid. ¹H NMR (DMSO-d₆, 400 MHz) δ_{H}=9.26 (s, 1H), 8.64 (d, J=4.4 Hz, 1H), 8.22 (d, J=9.2 Hz, 1H), 7.96-7.80 (m, 2H), 7.45-7.23 (m, 4H), 6 39 (d, J=8.4 Hz, 1H), 6.03 (s, 2H), 2.15 (s, 3H).

Step 2. A mixture of 6-methyl-N2-[2-nitro-5-(2-pyridyl)phenyl]pyridine-2,5-diamine (550 mg, 1.71 mmol), trans-4-methoxycarbonylcyclohexanecarboxylic acid (382 mg, 2.05 mmol) and EDCI (394 mg, 2.05 mmol) in pyridine (5 mL) was degassed and purged with N₂ (×3), and the mixture was stirred at 40° C. for 12 hrs under N₂ atmosphere. The reaction mixture was filtered and the filtrate was concentrated. The residue was dissolved in water (30 mL) and the resulting suspension was extracted with EtOAc (50 mL×3). The combined organic phase was washed with brine (30 mL), water (30 mL), dried over anhydrous Na₂SO₄, filtered and concentrated The residue was purified by Combi-Flash column (EtOAc in PE, 60%-80%) to give trans-methyl 4-[[2-methyl-6-[2-nitro-5-(2-pyridyl) anilino]-3-pyridyl]carbamoyl]cyclohexanecarboxylate (420 mg, 50.1% yield) as a yellow solid. LCMS Rₜ=0.632 min in 1 min chromatography, MS ESI calcd. for 489.2 [M+H]⁺ 490.2, found 490.2.

Step 3. To a solution of trans-methyl 4-[[2-methyl-6-[2-nitro-5-(2-pyridyl) anilino]-3-pyridyl]carbamoyl]cyclohexanecarboxylate (420 mg, 0.858 mmol) in THF (5 mL), wet Pd/C (0.1 g, purity 10%) was added under N₂ atmosphere. The suspension was degassed and purged with H₂ (×3) and the reaction mixture was stirred under H₂ (15 psi) at 25° C. for 16 hrs. The reaction mixture was filtered via a celite pad, and the filter cake was washed with DCM:MeOH (10:1, 100 mL). The filtrate was concentrated to give trans-methyl 4-[[6-[2-amino-5-(2-pyridyl)anilino]-2-methyl-3-pyridyl]carbamoyl]cyclohexanecarboxylate (380 mg, 96.4% yield) as a yellow solid. ¹H NMR (400 MHz, DMSO-d₆) δ=9.21 (s, 1H), 8.66-8.48 (m, 1H), 8.08 (s, 1H), 7.96-7.64 (m, 4H), 7 43-7 15 (m, 2H), 6.87 (d, J=8.4 Hz, 1H), 6.44 (d, J=8.4 Hz, 1H), 5.24 (s, 2H), 3.65 (s, 3H), 2.37 (s, 1H), 2.25 (s, 3H), 2.08-1.91 (m, 4H), 1.63-1.36 (m, 5H).

Step 4. To a solution of trans-methyl 4-[[6-[2-amino-5-(2-pyridyl)anilino]-2-methyl-3-pyridyl]carbamoyl]cyclohexanecarboxylate (380 mg, 0.827 mmol) in acetic acid (5 mL), 2-aminopyridine-3-carbaldehyde (151 mg, 1.24 mmol) was added and the reaction mixture was degassed and purged with 02 (×3) and the mixture was stirred at 80° C. for 2 hrs. The reaction mixture was concentrated to provide methyl (1r,4r)-4-((6-(2-(2-aminopyridin-3-yl)-6-(pyridin-2-yl)-1H-benzo[d]imidazol-1-yl)-2-methylpyridin-3-yl)carbamoyl)cyclohexane-1-carboxylate, which was used in the next step directly. LCMS Rₜ=0.359 min in 1 min chromatography, purity 87.4%, MS ESI calcd. for 561.25 [M+H]⁺ 562.25, found 562.2.

Step 5. To a solution of trans-methyl 4-[[6-[2-(2-amino-3-pyridyl)-6-(2-pyridyl) benzimidazol-1-yl]-2-methyl-3-pyridyl]carbamoyl]cyclohexanecarboxylate (280 mg, 0.499 mmol) in water (1 mL) and THF (4 mL), LiOH·H₂O (34.0 mg, 1.50 mmol) was added and the mixture was stirred at 25° C. for 12 hrs. The residue was purified by prep-HPLC (Column: Xtimate C18 200*40 mm*7 μm; mobile phase: [water(NH₄HCO₃)-ACN]; B %: 0%-38%. 25 min) to give (1r,4r)-4-((6-(2-(2-aminopyridin-3-yl)-6-(pyridin-2-yl)-1H-benzo[d]imidazol-1-yl)-2-methylpyridin-3-yl)carbamoyl)cyclohexane-1-carboxylic acid I-133 (22.0 mg, 8.06% yield) as a white solid. ¹H NMR (DMSO-d₆, 400 MHz) δ_{H}=9.57 (s, 1H), 8.67-8.59 (m, 1H), 8.16 (d, J=8.4 Hz, 1H), 8.08 (dd, J=1.6, 8.4 Hz, 1H), 8.04 (s, 1H), 8.02-7.98 (m, 2H), 7.91-7.83 (m, 2H), 7.41 (d, J=8.4 Hz, 1H), 7.32 (dd, J=5.2, 6.8 Hz, 1H), 7.14 (s, 2H), 7.08 (dd, J=1.6, 7.8 Hz, 1H), 6.42 (dd, J=4.8, 7.6 Hz, 1H), 2.47-2.41 (m, 4H), 2.26-2.17 (m, 1H), 1.98 (t, J=14.0 Hz, 4H), 1.57-1.45 (m, 2H), 1.44-1.32 (m, 2H). HPLC Rₜ=3.685 min in 8 min chromatography, purity 98.3%. LCMS Rₜ=1 735 min in 4 min chromatography, purity 96.6%, MS ESI calcd. for 547.23 [M+H]⁺ 548.23, found 548.2.

### Example 125. Synthesis of (1s,4s)-4-((5-(2-(2-aminopyridin-3-yl)-5-phenyl-3H-imidazo[4,5-b]pyridin-3-yl)-6-methylpyridin-2-yl)amino)cyclohexane-1-carboxylic acid (Compound I-134)

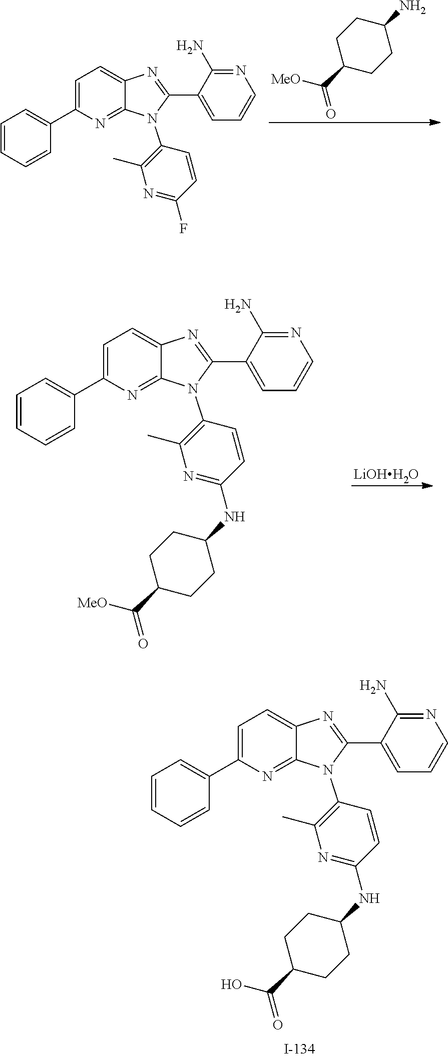

Step 1. To a solution of 3-[3-(6-fluoro-2-methyl-3-pyridyl)-5-phenyl-imidazo[4,5-b]pyridin-2-yl]pyridin-2-amine (300 mg, 0.757 mmol) in NMP (3 mL), methyl (1s,4s)-4-aminocyclohexane-1-carboxylate (220 mg, 1.14 mmol) and DIEA (0 40 mL, 2.27 mmol) were added, and the mixture was subjected to microwave irradiation at 150° C. for 4 hrs Water (10 mL) was added to the reaction mixture and the whole extracted with EtOAc (30 mL×3). The organic layer was dried over Na₂SO₄ and concentrated in vacuum to give a crude. The crude was purified by flash column (0-80% of EtOAc in PE) to give methyl (is, 4s)-4-((5-(2-(2-aminopyridin-3-yl)-5-phenyl-³H-imidazo[4,5-b]pyridin-3-yl)-6-methylpyridin-2-yl)amino)cyclohexane-1-carboxylate (170 mg, 42.1% yield) as a yellow oil. LCMS Rₜ=0.813 min in 1.5 min chromatography, purity 33.7%, MS ESI calcd. For 533.25 [M+H]⁺ 534.25, found 534.3.

Step 2. To a solution of methyl (is, 4s)-4-((5-(2-(2-aminopyridin-3-yl)-5-phenyl-3H-imidazo[4,5-b]pyridin-3-yl)-6-methylpyridin-2-yl)amino)cyclohexane-1-carboxylate (170 mg, 0.319 mmol) in water (2 mL) and THF (2 mL), LiOH·H₂O (40 mg, 0.956 mmol) was added and the reaction stirred at 25° C. for 16 hrs. The crude was purified by prep-HPLC (column: Welch Xtimate C18 150*30 mm*5 μm; mobile phase:water(FA)-ACN; B %:20%-50%, 30 min) to give (1s,4s)-4-((5-(2-(2-aminopyridin-3-yl)-5-phenyl-3H-imidazo[4,5-b]pyridin-3-yl)-6-methylpyridin-2-yl)amino)cyclohexane-1-carboxylic acid 1-134 (22.5 mg, 13.6% yield) as a yellow solid. ¹H NMR (DMSO-d₆, 400 MHz) δ_{H}=12.11 (s, 1H), 8.26 (d, J=8.4 Hz, 1H), 8.04-7.98 (m, 3H), 7.95 (d, J=8.0 Hz, 1H), 7.51-7.45 (m, 2H), 7.43-7.35 (m, 2H), 7.27 (dd, J=1.6, 7.6 Hz, 1H), 7.20 (s, 2H), 6.80 (d, J=7.2 Hz, 1H), 6.52-6.43 (m, 2H), 3.98-3.86 (m, 1H), 2.45-2.38 (m, 1H), 1.95-1.85 (m, 5H), 1.75-1.68 (m, 2H), 1.66-1.55 (m, 4H). HPLC Rₜ=3.706 min in 8 min chromatography, purity 100%. LCMS Rₜ=2.870 min in 4 min chromatography, purity 100%, MS ESI calcd. For 519.24 [M+H]⁺ 520.24, found 520.1.

### Example 126. Synthesis of 2-(1-((5-(2-(2-aminopyridin-3-yl)-5-phenyl-3H-imidazo[4,5-b]pyridin-3-yl)pyridin-2-yl)methyl)piperidin-4-yl)-2-methylpropanoic acid (Compound I-135)

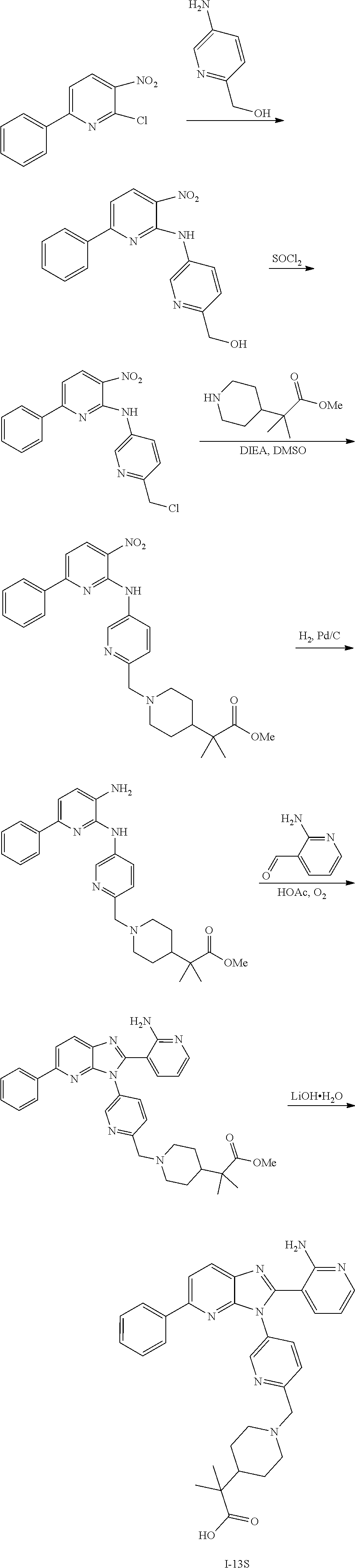

Step 1. To a solution of 2-chloro-3-nitro-6-phenylpyridine (5.00 g, 21.3 mmol) in DMSO (50.0 mL), (5-aminopyridin-2-yl)methanol (3.17 g, 25.6 mmol) and DIEA (4.13 g, 32.0 mmol) were added at 25° C. and the mixture was stirred at 80° C. for 14 hrs. Water (200 mL) was added to the mixture and the reaction mixture was filtered. The filter cake was concentrated. ACN (10 mL) was added, and the mixture stirred for 10 mins. The reaction mixture was filtered and the filter cake was concentrated to afford (5-((3-nitro-6-phenylpyridin-2-yl)amino)pyridin-2-yl)methanol (6.00 g, 87.4%) as a yellow solid. ¹H NMR (400 MHz, DMSO-d₆) δ=10.11 (s, 1H), 8.83 (d, J=2.4 Hz, 1H), 8.62 (d, J=8.8 Hz, 1H), 8.16-8.11 (m, 1H), 8.08-8.03 (m, 2H), 7.60-7.57 (m, 1H), 7.55-7.50 (m, 4H), 5 45-5.39 (t, J=6.0 Hz, 1H), 4.60 (d, J=6.0 Hz, 2H).

Step 2. A mixture of (5-((3-nitro-6-phenylpyridin-2-yl)amino)pyridin-2-yl)methanol (2.00 g, 6.22 mmol) and SOCl₂ (23 mL, 6.22 mmol) was stirred at 25° C. for 3 hrs. The reaction mixture was concentrated directly to give N-(6-(chloromethyl)pyridin-3-yl)-3-nitro-6-phenylpyridin-2-amine (1.30 g, 61.5%) as a yellow solid, which was used in the next step without purification. ¹H NMR (DMSO-d₆, 400 MHz) δ_{H}=10.18 (s, HH), 8.67 (d, J=8.8 Hz, 1H), 8.17-8.13 (m, 2H), 7.83 (d, J=8.8 Hz, 2H), 7.66 (d, J=8.4 Hz, 1H), 7.61-7.57 (m, 3H), 7.54 (d, J=8.4 Hz, 2H). 4.85 (s, 2H).

Step 3. To a solution of N-(6-(chloromethyl)pyridin-3-yl)-3-nitro-6-phenylpyridin-2-amine (250 mg, 0.730 mmol) in DMSO (5 mL), DIEA (381 mg, 2.93 mmol) and methyl 2-methyl-2-(piperidin-4-yl)propanoate (190 mg, 1.03 mmol) were added and the mixture was stirred at 80° C. for 14 hrs. Water (10 mL) was added to the mixture and the mixture was extracted with EtOAc (20 mL×3). The organic phase was washed with brine (20 mL), dried over Na₂SO₄, filtered, and concentrated under reduced pressure to provide the crude material. The residue was purified by Combi-Flash column (EtOAc in PE=0%-30%) to afford methyl 2-methyl-2-(1-((5-((3-nitro-6-phenylpyridin-2-yl)amino)pyridin-2-yl)methyl)piperidin-4-yl)propanoate (130 mg, 36.2% yield) as a yellow oil. ¹H NMR (400 MHz, DMSO-d₆) δ=10.17 (s, 1H), 8.88 (d, J=2.8 Hz, 1H), 8.69 (d, J=8.4 Hz, 1H), 8.17 (dd, J=2.8, 8.8 Hz, 1H), 8.13-8.08 (m, 2H), 7.68 (d, J=8.8 Hz, 1H), 7.62-7.56 (m, 3H), 7.53 (d, J=8.4 Hz, 1H), 3.65 (s, SH), 2.98 (d, J=11.2 Hz, 2H), 2.61 (s, 1H), 1.56-1.48 (m, 3H), 1.40-1 28 (m, 3H), 1.13 (s, 6H).

Step 4. To a solution of methyl 2-methyl-2-(1-((5-((3-nitro-6-phenylpyridin-2-yl)amino)pyridin-2-yl)methyl)piperidin-4-yl)propanoate (130 mg, 0.260 mmol) in methanol (5.00 mL), Pd/C (0.1 g, purity 10%) was added under N₂ atmosphere. The suspension was degassed and purged with H₂ (×3) and the reaction mixture was stirred under H₂ (15 psi) at 25° C. for 12 hrs. The reaction mixture was filtered with THF (20 mL), and the filtrate was concentrated to afford methyl 2-(1-((5-((3-amino-6-phenylpyridin-2-yl)amino)pyridine-2-yl)methyl)piperidin-4-yl)-2-methylpropanoate (90.0 mg, 73.8%) as a brown solid. ¹H NMR (400 MHz, DMSO-d₆) δ=8.96-8.86 (m, 1H), 8.23-8.18 (m, 1H), 8.14-8.06 (m, 1H), 8.03-7.91 (m, 2H), 7.49-7.39 (m, 2H), 7.37-7.24 (m, 3H), 7.07-7.00 (m, 1H), 5.33 (d, J=10.4 Hz, 2H), 3.66-3.59 (m, 3H), 3.01-2.85 (m, 2H), 2.46 (s, 1H), 2.40-2.35 (m, 1H), 2.01-1.91 (m, 1H), 1.54-1.37 (m, 4H), 1.28 (s, 2H), 1.13-1.07 (m, 6H).

Step 5. To a solution of methyl 2-(1-((5-((3-amino-6-phenylpyridin-2-yl)amino)pyridin-2-yl)methyl)piperidin-4-yl)-2-methylpropanoate (90.0 mg, 0.190 mmol) in acetic acid (2 mL), 2-aminonicotinaldehyde (36 0 mg, 0.29 mmol) was added. The reaction mixture was degassed and purged with 02 (×3) and the mixture was stirred at 80° C. for 2 hrs under 02. The reaction mixture was concentrated directly to afford methyl 2-(1-((5-(2-(2-aminopyridin-3-yl)-5-phenyl-3H-imidazo[4,5-b]pyridin-3-yl)pyridin-2-yl)methyl)piperidin-4-yl)-2-methylpropanoate (90.0 mg, 81.8% yield) as a brown oil.

Step 6. To a solution of methyl 2-(1-((5-(2-(2-aminopyridin-3-yl)-5-phenyl-3H-imidazo[4,5-b]pyridin-3-yl)pyridin-2-yl)methyl)piperidin-4-yl)-2-methylpropanoate (90.0 mg, 0.160 mmol) in THF (2 mL) and water (2 mL), LiOH·H₂O (98.0 mg, 2.40 mmol) was added at 25° C. and the mixture was stirred for 14 hrs. FA (2 mL) was added to the mixture. The residue was purified by prep-HPLC (condition: water (NH₄HCO)-ACN; Column: Xtimate C18 40*200 mm*7 μm; B %: 4%-44%; Gradient Time, 25 min) to afford 2-(1-((5-(2-(2-aminopyridin-3-yl)-5-phenyl-3H-imidazo[4,5-b]pyridin-3-yl)pyridin-2-yl)methyl)piperidin-4-yl)-2-methylpropanoic acid I-135 (31.2 mg, 34.7%) as a yellow solid. ¹H NMR (DMSO-d₆, 400 MHz) δ_{H}=12.32-11.88 (m, 1H), 8.64 (d, J=2.0 Hz, 1H), 8.30 (d, J=8.0 Hz, 1H), 8.08-7.99 (m, 4H), 7.93 (dd, J=2.8, 8.8 Hz, 1H), 7.61 (d, J=84 Hz, 1H), 7.52-7.45 (m, 2H), 7.44-7.38 (m, 1H), 7.30 (d, J=6.4 Hz, 1H), 6.80 (s, 2H), 6.47 (dd, J=4.8, 7.6 Hz, 1H), 3.66 (s, 2H), 2.98-2.85 (m, 2H), 2.01 (t, J=10.8 Hz, 2H), 1.51 (d, J=7.6 Hz, 3H), 1.40-1.25 (m, 2H), 1 04 (s, 6H). HPLC Rₜ=2.110 min in 8 min chromatography, purity 97.6%. LCMS Rₜ=1.928 min in 4 min chromatography, purity 98.1%, MS ESI calcd for 547.27 [M+H]⁺ 548.27, found 548.2.

### Example 127. Synthesis of 2-((5-(2-(2-aminopyridin-3-yl)-5-phenyl-3H-imidazo[4,5-b]pyridin-3-yl)pyridin-2-yl)methyl)-2-azaspiro[3.3]heptane-6-carboxylic acid (Compound I-136)

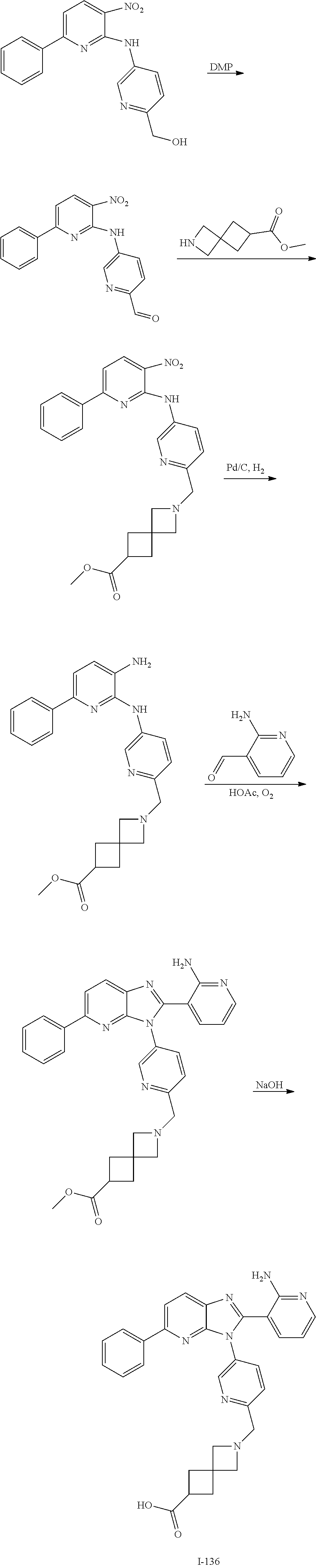

Step 1. To a solution of (5-((3-nitro-6-phenylpyridin-2-yl)amino)pyridin-2-yl)methanol (1.00 g, 3.10 mmol) in DCM (12 mL), DMP (2.63 g, 6.21 mmol) was added at 0° C. and the mixture was stirred at 25° C. for 2 hrs. The mixture was quenched with Na₂S₂O₃:NaHCO₃ (1/1), and further stirred for 30 mins. The reaction mixture was filtered and the filter cake was concentrated and triturated with DCM (10 mL) for 12 hrs. The reaction mixture was filtered and the filter cake was concentrated to afford 5-((3-nitro-6-phenylpyridin-2-yl)amino)picolinaldehyde (1.00 g, 74.5% yield) as a yellow solid. ¹H NMR (DMSO-d₆, 400 MHz) δ_{H}=10.15 (s, 1H), 8.83 (d, J=2.4 Hz, 1H), 8.63 (d, J=8.8 Hz, 1H), 8.15-8.06 (m, 31H), 7.65 (t, J=9.2 Hz, 2H), 7.59-7.51 (m, 3H).

Step 2. To a solution of 5-((3-nitro-6-phenylpyridin-2-yl)amino)picolinaldehyde (775 mg, 2.50 mmol) in methanol (3 mL), TEA (706 mg, 6.24 mmol) (pH-7) was added. Methyl 2-azaspiro[3.3]heptane-6-carboxylate (200 mg, 0.62 mmol) was then added and the mixture was stirred at 25° C. for 0.5 hr. To the mixture NaBH₃CN (118 mg, 1.87 mmol) and AcOH (2 drops) were added and the mixture was stirred at 25° C. for 0.5 hrs. Saturated aqueous NaHCO₃ (10 mL) was added to the mixture and the mixture was extracted with DCM (30 mL×3). The organic phase was dried over Na₂SO₄, filtered, and concentrated under reduced pressure to give a crude. The residue was purified by Combi-Flash column (MeOH in DCM, 0%-9%) to give methyl 2-((5-((3-nitro-6-phenylpyridin-2-yl)amino)pyridin-2-yl)methyl)-2-azaspiro[3.3]heptane-6-carboxylate (120 mg, 41.8% yield) as a yellow solid. ¹H NMR (400 MHz, DMSO-d₆) δ=10.09 (s, 1H), 8 88 (d, J=1.6 Hz, 1H), 8.79-8.71 (m, 1H), 8.56 (d, J=8.8 Hz, 1H), 8.19-8.12 (m, IH), 8.01-7.93 (m, 2H), 7.58 (d, J=8.8 Hz, 1H), 7.50-7.42 (m, 3H), 4.48-4.20 (m, 2H), 4.09-3.90 (m, 4H), 3.50 (s, 3H), 2.94 (d, J=8.0 Hz, 1H), 2.35-2.25 (m, 4H).

Step 3. To a solution of methyl 2-((5-((3-nitro-6-phenylpyridin-2-yl)amino)pyridin-2-yl)methyl)-2-azaspiro[3.3]heptane-6-carboxylate (100 mg, 0.260 mmol) in methanol (5.00 mL), wet Pd/C (0.1 g, purity 10%) was added under N₂ atmosphere. The suspension was degassed and purged with H₂ (×3) and the reaction mixture was stirred under H₂ (15 psi) at 25° C. for 12 hrs. The reaction mixture was filtered with THF (20 mL) and the filtrate was concentrated to afford methyl 2-((5-((3-amino-6-phenylpyridin-2-yl)amino)pyridine-2-yl)methyl)-2-azaspiro[3.3]heptane-6-carboxylate (80.0 mg, 85.6% yield) as a brown solid. ¹H NMR (400 MHz, DMSO-d₆) δ=8.98 (d, J=2.0 Hz, 1H), 8.29-8.13 (m, 2H), 7.90 (d, J=7.2 Hz, 2H), 7.46-7.23 (m, 5H), 7.04 (d, J=8.0 Hz, 1H), 5.32 (s, 2H), 3.59 (s, 3H), 2.45-2.39 (m, 3H), 2.37-2.29 (m, 4H), 1.31-1.20 (m, 4H).

Step 4. To a solution of methyl 2-((5-((3-amino-6-phenylpyridin-2-yl)amino)pyridine-2-yl)methyl)-2-azaspiro[3.3]heptane-6-carboxylate (80.0 mg, 0.180 mmol) in acetic acid (2 mL), 2-aminonicotinaldehyde (34.0 mg, 0.270 mmol) was added. The reaction mixture was degassed and purged with 02 (×3) and the mixture was stirred at 80° C. for 2 hrs. The reaction mixture was concentrated directly to afford methyl 2-((5-(2-(2-aminopyridin-3-yl)-5-phenyl-3H-imidazo[4,5-b]pyridin-3-yl)pyridin-2-yl)methyl)-2-azaspiro[3.3]heptane-6-carboxylate (80.0 mg, 80.8% yield) as a brown oil.

Step 5. To a solution of methyl 2-((5-(2-(2-aminopyridin-3-yl)-5-phenyl-3H-imidazo[4,5-b]pyridin-3-yl)pyridin-2-yl)methyl)-2-azaspiro[3.3]heptane-6-carboxylate (80.0 mg, 0.150 mmol) in THF (2 mL) and water (2 mL), NaOH (60.0 mg, 1.50 mmol) was added at 25° C. and the mixture was stirred at 60° C. for 14 hrs. FA (2 mL) was added to the mixture. The residue was purified by prep-HPLC (condition: water (NH₄HCO₃)-ACN: Column: Xtimate C18 40*200 mm*7 um; B %: 4%-44%; Gradient Time, 25 min) to afford 2-((5-(2-(2-aminopyridin-3-yl)-5-phenyl-3H-imidazo[4,5-b]pyridin-3-yl)pyridin-2-yl)methyl)-2-azaspiro[3.3]heptane-6-carboxylic acid I-136 (9.30 mg, 11.9% yield) as a yellow solid. ¹H NMR (DMSO-d₆, 400 MHz) δ=8.62 (d, J=2.0 Hz, 1H), 8.29 (d, J=8.4 Hz, 1H), 8.09-7.99 (m, 4H), 7.91 (dd, J=2.4, 8.0 Hz, 1H), 7.54-7.45 (m, 3H), 7.44-7.38 (m, 1H), 7.32-7.26 (m, 1H), 6.77 (s, 2H), 6.49 (dd, J=4.8, 7.2 Hz, 1H), 3.70 (s, 2H), 3.26-3.25 (m, 1H), 3.20 (s, 2H), 2.92 (s, 1H), 2.65-2.56 (m, 1H), 2.32-2.20 (m, 4H). HPLC Rₜ=2.810 min in 8 min chromatography, purity 99.4%. LCMS Rₜ=1.252 min in 4 min chromatography, purity 99.8%, MS ESI calcd. for 517.22 [M+H]⁺ 518.22, found 518.2.

### Example 128. Synthesis of 6-(((5-(2-(2-aminopyridin-3-yl)-5-phenyl-3H-imidazo[4,5-b]pyridin-3-yl)pyridin-2-yl)amino)methyl)spiro[3.3]heptane-2-carboxylic acid (Compound I-137)

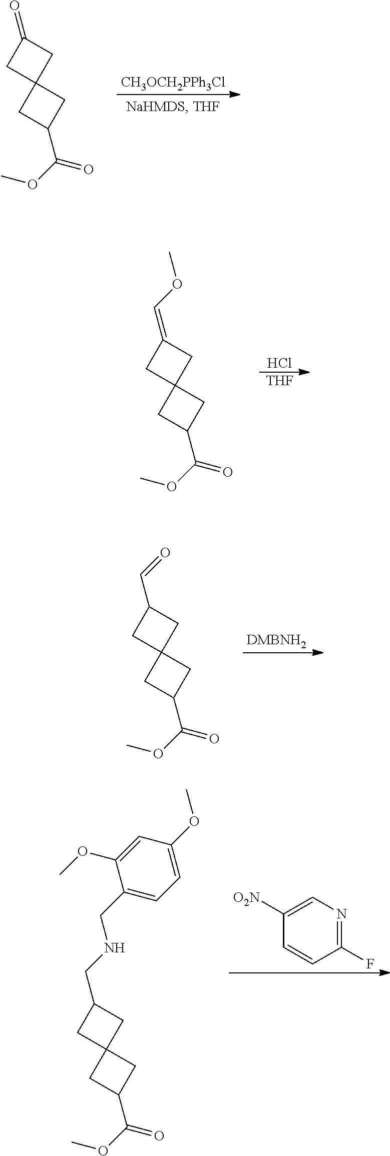

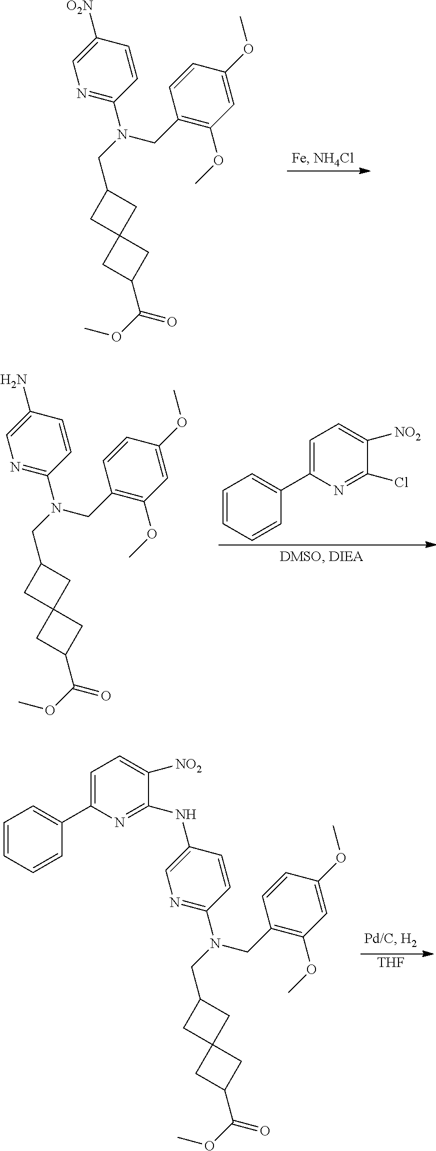 [SMILES: "THF" ; COC(=O)C1CC2(CC(CN(Cc3ccc(OC)cc3OC)c3ccc([N+](=O)[O-])cn3)C2)C1 ; COC(=O)C1CC2(CC(CN(Cc3ccc(OC)cc3OC)c3ccc(N)cn3)C2)C1 ; O=[N+]([O-])c1ccc(-c2ccccc2)nc1Cl ; COC(=O)C1CC2(CC(CN(Cc3ccc(OC)cc3OC)c3ccc(Nc4nc(-c5ccccc5)ccc4[N+](=O)[O-])cn3)C2)C1]

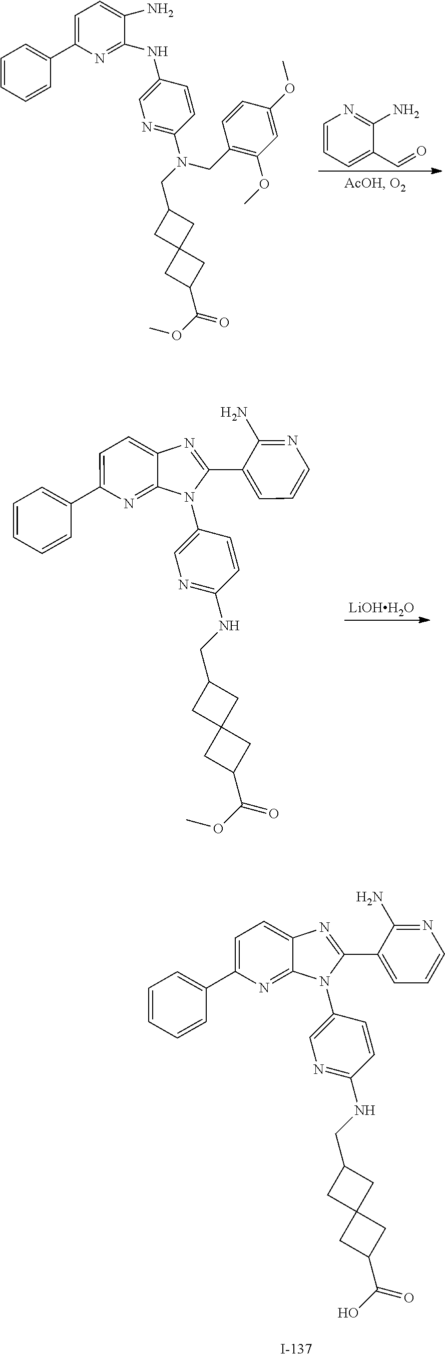 [SMILES: COC(=O)C1CC2(CC(CN(Cc3ccc(OC)cc3OC)c3ccc(Nc4nc(-c5ccccc5)ccc4N)cn3)C2)C1 ; Nc1ncccc1C=O ; COC(=O)C1CC2(CC(CNc3ccc(-n4c(-c5cccnc5N)nc5ccc(-c6ccccc6)nc54)cn3)C2)C1 ; "LiOH·H2O" ; Nc1ncccc1-c1nc2ccc(-c3ccccc3)nc2n1-c1ccc(NCC2CC3(C2)CC(C(=O)O)C3)nc1]

Step 1. To a solution of CH₃OCH₂PPh₃Cl (15.3 g, 44.6 mmol) in THF (30.0 mL), NaHMDS (45.0 mL, 44.6 mmol) was added slowly at −40° C. for 10 mins under N₂ atmosphere. After addition, the mixture was stirred at −40° C. for 1 hr and at 0° C. for 30 mins. After cooling to −40° C., a solution of methyl 2-oxospiro[3.3]heptane-6-carboxylate (5.00 g, 29.7 mmol) in THF (30 mL) was then added, dropwise, at −40° C. The resulting mixture was stirred at 0° C. for 30 min under N₂ atmosphere. The mixture was quenched with aqueous saturated NH₄Cl (100 mL), extracted with EtOAc (200 mL×3) and washed with aqueous saturated NaHCO₃ (100 mL). The combined organic phase was washed with brine (100 mL), dried over anhydrous Na₂SO₄, filtered and concentrated. The residue was purified by Combi-Flash column (EtOAc in PE=0%-15%) to give methyl 2-(methoxymethylene) spiro[3.3]heptane-6-carboxylate (1.00 g, 17.1% yield) as a colorless oil. ¹H NMR (DMSO-d₆, 400 MHz) β_{H}=5.75 (td, J=2.0, 6.4 Hz, 1H), 3.49 (s, 3H), 3.37 (d, J=2.0 Hz, 3H), 2.92 (dt, J=1.2, 8.4 Hz, 1H), 2.62-2.43 (m, 4H), 2.16-2.05 (m, 4H).

Step 2. To a solution of methyl 2-(methoxymethylene) spiro[3.3]heptane-6-carboxylate (1.00 g, 5.10 mmol) in THF (5.00 mL), HCl (6.30 mL, 6 25 mmol) was added under N₂ atmosphere and the mixture was stirred at 20° C. for 3 hrs. Water (10 mL) was added to the mixture, and the whole extracted with ethyl acetate (25 mL×3). The organic phases were combined, and then washed successively with saturated sodium bicarbonate solution (20 mL), brine (20 mL), dried over anhydrous sodium sulfate, and concentrated to give methyl 6-formylspiro[3.3]heptane-2-carboxylate (800 mg, 86.2% yield) as a colorless oil. ¹H NMR (DMSO-d₆, 400 MHz) δ_{H}=9.63 (d, J=2.0 Hz, 1H), 3.60 (s, 3H), 3.04-2.85 (m, 2H), 2.33-2.04 (m, 8H).

Step 3. To a solution of methyl 6-formylspiro[3.3]heptane-2-carboxylate (800 mg, 4.39 mmol) in methanol (10 mL), (2,4-dimethoxyphenyl)methanamine (1.17 g, 7.02 mmol) (pH-7) was added and the mixture was stirred at 25° C. for 1 hr. NaBH₃CN (828 mg, 13.2 mmol) was then added, and the mixture was stirred at 25° C. for 2 hrs. The reaction was taken up in EtOAc (100 mL) and the organics were washed with water (50 mL) and subsequently washed with brine (50 mL×3). The organics were then separated and dried (Na₂SO₄) before concentrated to dryness. The crude was then purified by flash column chromatography eluting with 100% of EtOAc in Isohexane to give methyl 6-(((2,4-dimethoxybenzyl)amino)methyl)spiro[3.3]heptane-2-carboxylate (1.42 g, 61.1% yield) as a yellow oil.

Step 4. To a solution of methyl 6-(((2,4-dimethoxybenzyl)amino)methyl)spiro[3.3]heptane-2-carboxylate (1.00 g, 3.00 mmol) in DMSO (5.00 mL), 2-fluoro-5-nitro-pyridine (426 mg, 3.00 mmol) was added under N₂ atmosphere and the mixture was stirred at 90° C. for 6 hrs. The reaction was taken up in EtOAc (100 mL), and the organics washed with water (50 mL) and brine (50 mL×3). The organics were then separated and dried (Na₂SO₄) before concentration to dryness. The crude was then purified by flash column chromatography eluting with 70/o of EtOAc in Isohexane to give methyl 6-(((2,4-dimethoxybenzyl)(5-nitropyridin-2-yl)amino)methyl)spiro[3.3]heptane-2-carboxylate (640 mg, 46.9% yield) as a colorless oil. ¹H NMR (DMSO-d₆, 400 MHz) δ_{H}=8.88 (d, J=2.4 Hz, 1H), 8.09 (dd, J=2.4, 9.6 Hz, 1H), 6.72 (d, J=8.4 Hz, 1H), 6.63-6.47 (m, 2H), 6.37 (dd, J=2.4, 8.4 Hz, 1H), 4.61 (s, 2H), 3.74 (s, 3H), 3.66 (s, 3H), 3.50 (s, 3H), 2.92 (t, J=8.4 Hz, 1H), 2.47 (s, 2H), 2.14 (dd, J=2.4, 8.4 Hz, 1H), 2.07-1.99 (m, 4H), 1.89-1.82 (m, 1H), 1.74 (dd, J=8.4, 10.8 Hz, 1H), 1.65 (dd, J=8.4, 11.2 Hz, 1H).

Step 5. To a solution of methyl 6-(((2,4-dimethoxybenzyl)(5-nitropyridin-2-yl)amino)methyl)spiro[3.3]heptane-2-carboxylate (640 mg, 1.41 mmol) in ethanol (5 mL) and water (5 mL), Fe (393 mg, 7.03 mmol), NH₄Cl (376 mg, 7.03 mmol) were added under N₂ atmosphere. The reaction mixture was degassed and purged with N₂ (×3) and the mixture was stirred at 100° C. for 6 hrs. The reaction mixture was filtered and extracted with EtOAc (100 mL×3). The combined organic phase was washed with brine (50 mL), dried over anhydrous Na₂SO₄, filtered and concentrated. The crude was then purified by flash column chromatography eluting 80% EtOAc in Isohexane to give methyl 6-(((5-aminopyridin-2-yl)(2,4-dimethoxybenzyl)amino)methyl)spiro[3.3]heptane-2-carboxylate (330 mg, 55.2% yield) as a colorless oil. ¹H NMR (DMSO-d₆, 400 MHz) δ_{H}=7.52 (d, J=2.8 Hz, 1H), 6.85-6.79 (m, 1H), 6.74 (d, J=8.4 Hz, 1H), 6.54 (d, J=2.4 Hz, 1H), 6.38 (dd, J=2.4, 8.4 Hz, 1H), 6.22 (d, J=8.8 Hz, 1H), 4.41 (s, 1H), 4.36-4.22 (m, 1H), 3.80 (s, 3H), 3.73-3.70 (m, 3H), 3.58-3.55 (m, 3H), 3.02-2.92 (m, 1H), 2 46-2.34 (m, 2H), 2.32-2.14 (m, 2H), 2.14-2.04 (m, 4H), 1.93-1.84 (m, 1H), 1.73 (dd, J=8.0, 10.4 Hz, 1H), 1.66-1.58 (m, 1H).

Step 6. To a solution of methyl 6-(((5-aminopyridin-2-yl)(2,4-dimethoxybenzyl)amino)methyl)spiro[3.3]heptane-2-carboxylate (250 mg, 0.588 mmol) in DMSO (1.00 mL), DIEA (0.420 mL, 1.76 mmol) and 2-chloro-3-nitro-6-phenyl-pyridine (179 mg, 0.764 mmol) were added under N₂ atmosphere. The reaction mixture was degassed and purged with N₂ (×3) and the mixture was stirred at 80° C. for 6 hrs. The reaction was taken up in EtOAc (100 mL) and the organics washed with water (50 mL) and brine (50 mL×3). The organics were then separated and dried (Na₂SO₄) before concentration to dryness. The crude was then purified by flash column chromatography eluting with 50-80% of EtOAc in Isohexane to give methyl 6-[[(2,4-dimethoxyphenyl)methyl-[5-[(3-nitro-6-phenyl-2-pyridyl) amino]-2-pyridyl]amino]methyl]spiro[3.3]heptane-2-carboxylate (220 mg, 60.0% yield) as a yellow oil. ¹H NMR (DMSO-d₆, 400 MHz) δ_{H}=9.91 (s, 1H), 8.56 (d, J=8.4 Hz, 1H), 8.27 (d, J=2.4 Hz, IH), 8.01 (d, J=7.2 Hz, 2H), 7.70 (dd, J=2.4, 9.2 Hz, 1H), 7.55-7.39 (m, 5H), 6.81 (d, J=8.4 Hz, 1H), 6.59 (d, J=2.0 Hz, 1H), 6.53 (d, J=9.2 Hz, 1H), 6.43 (dd, J=2.0, 8.4 Hz, 1H), 4.58 (s, 2H), 3.83 (s, 3H), 3.73 (s, 3H), 3.65 (d, J=4.8 Hz, 1H), 3.61 (d, J=7.2 Hz, 2H), 3.57 (s, 3H), 2.99 (t, J=8.4 Hz, 1H), 2 16-2.07 (m, 4H), 1.96 (s, 1H), 1.82 (dd, J=8.4, 10 4 Hz, 1H), 1.73 (dd, J=8.4, 11.2 Hz, 1H).

Step 7. To a solution of methyl 6-[[(2,4-dimethoxyphenyl)methyl-[5-[(3-nitro-6-phenyl-2-pyridyl) amino]-2-pyridyl]amino]methyl]spiro[3.3]heptane-2-carboxylate (200 mg, 0.321 mmol) in THE (5 mL), wet Pd/C (0.100 g, purity 10%) was added under N₂ atmosphere. The suspension was degassed and purged with H₂ (×3) and the reaction mixture was stirred under H2 (15 psi) at 25° C. for 16 hrs. The reaction mixture was filtered via a celite pad, and the filter cake was washed with DCM:MeOH (10:1, 50 mL). The filtrate was concentrated to provide methyl 6-[[[5-[(3-amino-6-phenyl-2-pyridyl) amino]-2-pyridyl]-[(2,4-dimethoxyphenyl) methyl]amino]methyl]spiro[3.3]heptane-2-carboxylate (80.0 mg, 42.0% yield) as a yellow solid. ¹H NMR (DMSO-d₆, 400 MHz) δ_{H}=8.43 (d, J=2.4 Hz, 1H), 7.92-7.88 (m, 2H), 7.61 (s, 1H), 7.42-7.37 (m, 2H), 7.31-7.25 (m, 1H), 7.20 (d, J=8.0 Hz, 1H), 6.96 (d, J=8.0 Hz, 1H), 6.87 (d, 1=8.4 Hz, 1H), 6.64 (d, J=2.4 Hz, 1H), 6.53 (d, J=9.2 Hz, 1H), 6.47 (dd, J=2.4, 8.4 Hz, 1H), 5.20 (s, 2H), 4.59 (s, 2H), 3.88 (s, 3H), 3.78 (s, 3H), 3.65-3.59 (m, 5H), 3.42 (d, J=1.2 Hz, IH), 3.05 (t, J=8.4 Hz, 1H), 2.32-2.21 (m, 2H), 2.21-2.14 (m, 4H), 2.03-1.97 (m, 1H), 1.86 (dd, J=8.4, 10.0 Hz, 1H), 1.76 (dd, J=8.4, 11.2 Hz, 1H).

Step 8. To a solution of methyl 6-[[[5-[(3-amino-6-phenyl-2-pyridyl) amino]-2-pyridyl]-[(2,4-dimethoxyphenyl)methyl]amino]methyl]spiro[3.3]heptane-2-carboxylate (60.0 mg, 0.101 mmol) in acetic acid (1 mL), 2-aminopyridine-3-carbaldehyde (19.0 mg, 0.152 mmol) was added under 02 atmosphere. The reaction mixture was degassed and purged with 02 (×3) and the mixture was stirred at 100° C. for 1 hr. The reaction mixture was concentrated to provide crude methyl 6-[[[5-[2-(2-amino-3-pyridyl)-5-phenyl-imidazo[4,5-b]pyridin-3-yl]-2-pyridyl]amino]methyl]spiro[3.3]heptane-2-carboxylate as a brown oil, which was used in the next step directly.

Step 9. To a solution of methyl 6-[[[5-[2-(2-amino-3-pyridyl)-5-phenyl-imidazo[4,5-b]pyridin-3-yl]-2-pyridyl]amino]methyl]spiro[3.3]heptane-2-carboxylate (100 mg, 0.183 mmol) in THF (1.5 mL) and water (0.5 mL), LiOH·H₂O (13.0 mg, 0.550 mmol) was added under N₂ atmosphere and the mixture was stirred at 20° C. for 6 hrs. The crude product was purified by prep-HPLC (column. Welch Xtimate C18 150*30 mm*5 μm; mobile phase: [water (NH₄HCO₃)-ACN]; B %: 8%-48%, 25 min) to give 6-[[[5-[2-(2-amino-3-pyridyl)-5-phenyl-imidazo[4,5-b]pyridin-3-yl]-2-pyridyl]amino]methyl]spiro[3.3]heptane-2-carboxylic acid I-137 (15.1 mg, 15.3% yield) as a white solid. ¹H NMR (DMSO-d₆, 400 MHz) δ=8.24 (d, J=8.4 Hz, 1H), 8.08-7.99 (m, 4H), 7.97 (d, J=8.4 Hz, 1H), 7.52-7.44 (m, 3H), 7.43-7.36 (m, 2H), 6.98-6.86 (m, 3H), 6.58 (d, J=8.8 Hz, 1H), 6.50 (dd, J=4.8, 7.6 Hz, 1H), 3.27 (s, 2H), 2.96-2.84 (m, 1H), 2.41-2.36 (m, 1H), 2.25-2.14 (m, 3H), 2.11 (d, J=8.4 Hz, 2H), 2.02 (ddd, J=2.8, 8.0, 11.2 Hz, 1H), 1 78 (dd, J=7.6, 10.8 Hz, 1H), 1.69 (dd, J=7.6, 11.6 Hz, 1H). HPLC Rₜ=2.213 min in 8 min chromatography, 10-80AB, purity 98.9%. LCMS Rₜ=1.515 min in 4 min chromatography, Xtimate C18 2.1*50 mm, 3 μm, purity 98.4%, MS ESI calcd. for 531.24 [M+H]⁺ 532.24, found 532.2.

### Example 129. Synthesis of (1r,4r)-4-((3-(4-(2-(2-aminopyridin-3-yl)-5-phenyl-3H-imidazo[4,5-b]pyridin-3-yl)phenyl)azetidin-1-yl)methyl)-1-methylcyclohexane-1-carboxylic acid (Compound I-138)

### Synthesis of methyl 4-formyl-1-methylcyclohexane-1-carboxylate

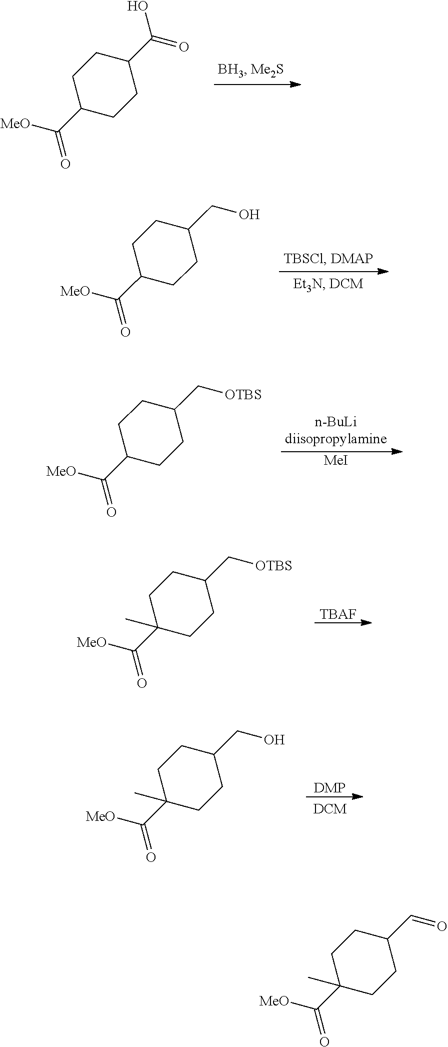

Step 1. To a solution of 4-methoxycarbonylcyclohexanecarboxylic acid (1.30 g, 6.98 mmol) in THF (15.0 mL), BH₃·Me₂S (15.0 mL, 15.0 mmol) was added at 0° C. under N₂ atmosphere. The suspension was degassed and purged with N₂ (×3), and the reaction mixture was stirred at 25° C. for 2 hrs. The reaction was quenched with MeOH (40 mL) dropwise, and the resulting mixture was extracted with EtOAc (30 mL×3). The combined organic phase was washed with brine (15 mL), water (15 mL), dried over anhydrous Na₂SO₄, filtered, and concentrated to give methyl 4-(hydroxymethyl) cyclohexanecarboxylate (1.00 g, 5.81 mmol, 83.17% yield) as a light-yellow oil, which was used directly in the next step. ¹H NMR (400 MHz, CDCl₃) δ=3.66 (s, 3H), 3.46 (d, J=6.4 Hz, 2H), 2.24 (tt, J=12.4, 3.6 Hz, 1H), 2.05-1.98 (m, 2H), 1.87 (dd, J=13.2, 2.4 Hz, 2H), 1.52-1.38 (m, 3H), 0.98 (qd, J=12.8, 3.6 Hz, 2H).

Step 2. To a solution of methyl 4-(hydroxymethyl)cyclohexanecarboxylate (1.00 g, 5.81 mmol) in DCM (15.0 mL), TEA (2.50 mL, 17.4 mmol) and DMAP (35.0 mg, 0.290 mmol) were added at 25° C., and the reaction mixture was stirred at 25° C. for 0.5 hr. Tert-butyl-chloro-dimethylsilane (1.14 g, 7.55 mmol) was subsequently added dropwise at 0° C., and the reaction mixture was stirred at 25° C. for a further 12 hrs. Water (30 mL) was added to the residue, and the resulting mixture was extracted with DCM (20 mL×3). The combined organic phase was washed with brine (10 mL), water (10 mL), dried over anhydrous Na₂SO₄, filtered, and concentrated. The residue was purified by flash silica gel chromatography (eluent of 0-9% EtOAc in PE) to give methyl 4-(((tert-butyldimethylsilyl)oxy)methyl)cyclohexane-1-carboxylate (1.60 g, 96.2% yield) as a colorless oil ¹H NMR (400 MHz, CDCl₃) δ=3.66 (s, 3H), 3.40 (d, J=6.4 Hz, 2H), 2.23 (tt, J=12.4, 3.6 Hz, 1H), 2.03-1.95 (m, 2H), 1.83 (dd, J=13.6, 3.2 Hz, 2H), 1.48-1.36 (m, 3H), 0.98-0.91 (m, 2H), 0.89 (s, 9H), 0.06-0.00 (m, 6H).

Step 3. To a solution of diisopropylamine (1.60 mL, 11.2 mmol) in THF (16.0 mL), n-BuLi (2.50 mL, 9.49 mmol) was added slowly at 0° C., and the mixture was subsequently stirred at 0° C. for 0.5 hr. A solution of methyl 4-(((tert-butyldimethylsilyl)oxy)methyl)cyclohexane-1-carboxylate (1.60 g, 5.58 mmol) in THF (8.00 mL) was then added dropwise at −78° C. and the reaction mixture was stirred at 0° C. for 1 hr. MeI (0.700 mL, 11.2 mmol) was then added dropwise, and upon addition completion, the mixture was stirred at −78° C. for an additional 1 hr, and subsequently stirred at 25° C. for 16 hrs. Aq. NH₄Cl (20 mL) was then added and the resulting mixture was extracted with EtOAc (30 mL×3). The combined organic phase was washed with brine (10 mL), water (10 mL), dried over anhydrous Na₂SO₄, filtered and concentrated. Compound methyl 4-(((tert-butyldimethylsilyl)oxy)methyl)-1-methylcyclohexane-1-carboxylate (1.20 g, 71 5% yield) was obtained as a yellow oil ¹H NMR (400 MHz, CDCl₃) δ=3.67 (s, 3H), 3.36 (d, J=6.4 Hz, 2H), 2.20 (d, J=12.0 Hz, 2H), 1.69-1.64 (m, 2H), 1.19-1.07 (m, 6H), 0.98-0 92 (m, 2H), 0.88-0.87 (m, 9H), 0 02 (s, 6H).

Step 4. To a solution of methyl 4-(((tert-butyldimethylsilyl)oxy)methyl)-1-methylcyclohexane-1-carboxylate (1.10 g, 3.66 mmol) in THF (10.0 mL), TBAF/THF (4.00 mL, 4.03 mmol) was added at 25° C., and the mixture was stirred for 3 hrs. The reaction mixture was concentrated directly. The residue was purified by flash silica gel chromatography (eluent of 0-60% EtOAc in PE) to give methyl 4-(hydroxymethyl)-1-methylcyclohexane-1-carboxylate (600 mg, 88.0% yield) as a yellow oil. ¹H NMR (400 MHz, CDCl₃) δ=3.67 (s, 3H), 3.42 (d, J=6.4 Hz, 2H), 2.27-2.18 (m, 2H), 1 72-1 67 (m, 2H), 1.21-1.09 (m, 6H), 1 05-0.95 (m, 2H).

Step 5. To a solution of methyl 4-(hydroxymethyl)-1-methyl-cyclohexanecarboxylate (300 mg, 1.61 mmol) in DCM (10.0 mL), DMP (1.02 g, 1.61 mmol) was added at 0° C., and the mixture stirred at 25° C. for 2 hrs. The reaction was quenched with aq. Na₂S₂O₄ (20 mL) and aq. NaHCO₃ (20 mL) dropwise. The resulting mixture was extracted with DCM (10 mL×3), and the combined organic phase was washed with brine (5 mL), water (5 mL), dried over anhydrous Na₂SO₄ filtered, and concentrated to give methyl 4-formyl-1-methyl-cyclohexanecarboxylate (200 mg, 67.4% yield) as a yellow oil, which was used in the next step directly.

### Synthesis of (1r,4r)-4-((3-(4-(2-(2-aminopyridin-3-yl)-5-phenyl-3H-imidazo[4,5-b]pyridin-3-yl)phenyl)azetidin-1-yl)methyl)-1-methylcyclohexane-1-carboxylic acid (Compound I-138)

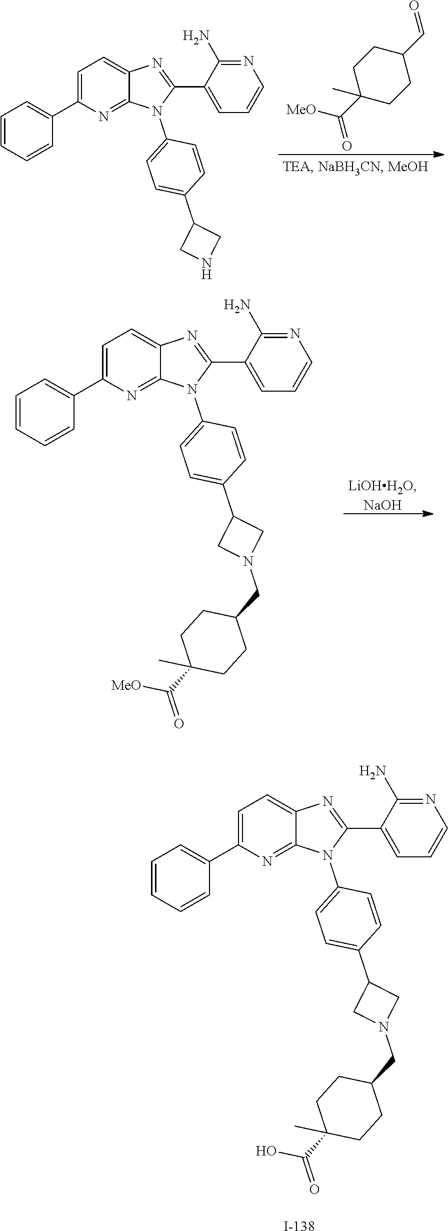

Step 1. To a solution of methyl 4-formyl-1-methyl-cyclohexanecarboxylate (200 mg, 1.09 mmol) in methanol (5.00 mL), TEA (330 mg, 3.26 mmol), 3-[3-[4-(azetidin-3-yl)phenyl]-5-phenyl-imidazo[4,5-b]pyridin-2-yl]pyridin-2-amine (454 mg, 1.09 mmol) and NaBH₃CN (102 mg, 1.63 mmol) were added at 25° C., and the mixture stirred for 16 hrs. The reaction mixture was concentrated directly. The residue was purified by flash silica gel chromatography (eluent of 0-4% MeOH in DCM) and purified by SFC (column: DAICEL CHIRALPAK AD (250 mm*30 mm, 10 μm); condition: CO₂-EtOH(0.1% NH₄H₂O), begin b. 60-60) to give methyl (1r,4r)-4-((3-(4-(2-(2-aminopyridin-3-yl)-5-phenyl-3H-imidazo[4,5-b]pyridin-3-yl)phenyl)azetidin-1-yl)methyl)-1-methylcyclohexane-1-carboxylate (400 mg, 62 8% yield) as a yellow oil. LCMS Rₜ=0.740 min in 2 min chromatography, purity 91.63%, MS ESI calcd. For 586.31, [M+H]⁺ 587.31, found 587.4. ¹H NMR (400 MHz, DMSO-d₆) δ=8.26 (d, J=8.4 Hz, 1H), 8.06-7.93 (m, 4H), 7.55-7.35 (m, 7H), 7.20 (d, J=7.6 Hz, 1H), 6.94 (s, 2H), 6.42 (dd, J=7.6, 4.8 Hz, IH), 3.73-3.55 (m, 6H), 3.08 (t, J=6.0 Hz, 2H), 2.25 (d, J=6.8 Hz, 2H), 2.07 (d, J=12.8 Hz, 2H), 1.65 (d, J=11.2 Hz, 2H), 1.20-1.00 (m, 6H), 0.95-0.79 (m, 2H).

Step 2. To a solution of methyl (1r,4r)-4-((3-(4-(2-(2-aminopyridin-3-yl)-5-phenyl-3H-imidazo[4,5-b]pyridin-3-yl)phenyl)azetidin-1-yl)methyl)-1-methylcyclohexane-1-carboxylate (100 mg, 0.170 mmol) in THF (2.00 mL), methanol (1.00 mL) and water (1.00 mL), LiOH·H₂O (40.0 mg, 0.976 mmol) and NaOH (240 mg, 6.00 mmol) were added at 25° C., and the reaction mixture was stirred at 40° C. for 24 hrs. The reaction mixture was concentrated to give crude product. The residue was purified by prep-HPLC (column: Welch Xtimate C18 40*200 mm 7 um; condition: water (NH₄HCO)-ACN; begin b: 10-50%) to give (1r,4r)-4-((3-(4-(2-(2-aminopyridin-3-yl)-5-phenyl-3H-imidazo[4,5-b]pyridin-3-yl)phenyl)azetidin-1-yl)methyl)-1-methylcyclohexane-1-carboxylic acid I-138 (14.0 mg, 14.7% yield) as a white solid. ¹H NMR (400 MHz, DMSO-d₆) δ=8.26 (d, J=8.4 Hz, 1H), 8.06-7.93 (m, 4H), 7.54-7.34 (m, 7H), 7.19 (d, J=7.6 Hz, 1H), 6.95 (s, 2H), 6.42 (dd, J=7.6, 4.8 Hz, 1H), 3.70-3.60 (m, 3H), 3.08 (t, 1=6.0 Hz, 2H), 2.25 (d, J=6.8 Hz, 2H), 2.04 (d, J=12.0 Hz, 2H), 1.62 (d, J=10.8 Hz, 2H), 1.28-1.19 (m, 1H), 1.10-0.90 (m, 7H). HPLC Rₜ=2.261 min in 8 min chromatography, purity 99.85%. LCMS Rₜ=0.721 min in 2 min chromatography, purity 99.93%, MS ESI calcd. for 572.29, [M+H]⁺ 573.29, found 573.3.

### Example 130. Synthesis of (1r,4r)-4-((3-(4-(2-(2-aminopyridin-3-yl)-5-phenyl-3H-imidazo[4,5-b]pyridin-3-yl)-3-methylphenyl)azetidin-1-yl)methyl)cyclohexane-1-carboxylic acid (Compound (1-139)

### Synthesis of tert-butyl 3-(4-amino-3-methylphenyl)azetidine-1-carboxylate

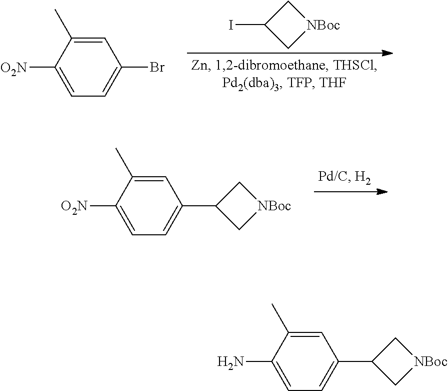 [SMILES: Cc1cc(Br)ccc1[N+](=O)[O-] ; CC(C)(C)OC(=O)N1CC(I)C1 ; Cc1cc(C2CN(C(=O)OC(C)(C)C)C2)ccc1[N+](=O)[O-] ; Cc1cc(C2CN(C(=O)OC(C)(C)C)C2)ccc1N]

Step 1. Zn (1.79 g, 27.5 mmol) was vigorously stirred in THF (24 mL), under nitrogen, and 1,2-dibromoethane (498 mg, 2.65 mmol) was added. The suspension was then heated at 80° C. for 8 min and subsequently allowed to cool to room temperature. TMSCl (288 mg, 2.65 mmol) in THF (12 mL) was then added and the mixture further stirred at 25° C. for 45 min. A solution of tert-butyl 3-iodoazetidine-1-carboxylate (5.00 g, 17.7 mmol) in THF (24 mL) was then added, dropwise, to the solution over a 15 min period, and the reaction mixture stirred at room temperature for 2 hrs. Pd₂(dba)₃ (323 mg, 0.353 mmol) and tri(2-furyl)phosphine (246 mg, 1.06 mmol) were then added to the mixture, followed by 4-bromo-2-methyl-1-nitro-benzene (4.20 g, 19.4 mmol) in THF (48 mL). The mixture was then heated at 55° C. for 3 hrs, cooled to room temperature and quenched with brine (200 mL). The mixture was then extracted with dichloromethane (300 mL×3), the organic phase dried with Na₂SO₄, filtered, and evaporated in vacuo to provide the crude material. The crude material was purified by flash column chromatography (PE:EA=3:1) to give the title compound tert-butyl 3-(3-methyl-4-nitro-phenyl)azetidine-1-carboxylate (3.80 g, 73.60% yield) as a brown oil. ¹H NMR (DMSO-d₆) δ ppm 7.98 (d, J=8.4 Hz, 1H), 7.49 (s, 1H), 7.42 (dd, J=8.4, 1.6 Hz, 1H), 4.27 (t, J=6.4 Hz, 2H), 3.78-3.93 (m, 3H), 2.54 (s, 3H), 1.41 (s, 9H).

Step 2. To a solution of tert-butyl 3-(3-methyl-4-nitro-phenyl)azetidine-1-carboxylate (3.80 g, 13.0 mmol) in THF (40 mL) was added wet Pd/C (1.00 g, purity 10%) under N₂ atmosphere. The suspension was degassed and purged with H₂ (×3), and the reaction mixture was stirred under H₂ (50 Psi) at 50° C. for 16 hrs. After cooling to room temperature, the mixture was filtered through celite. The filtrate was concentrated to provide tert-butyl 3-(4-amino-3-methyl-phenyl)azetidine-1-carboxylate (3.40 g, crude) as a yellow oil, which was used directly in the next step without further purification.

### Synthesis of (1r,4r)-4-((3-(4-(2-(2-aminopyridin-3-yl)-5-phenyl-3H-imidazo[4,5-b]pyridin-3-yl)-3-methylphenyl)azetidin-1-yl)methyl)cyclohexane-1-carboxylic acid (Compound (1-139)

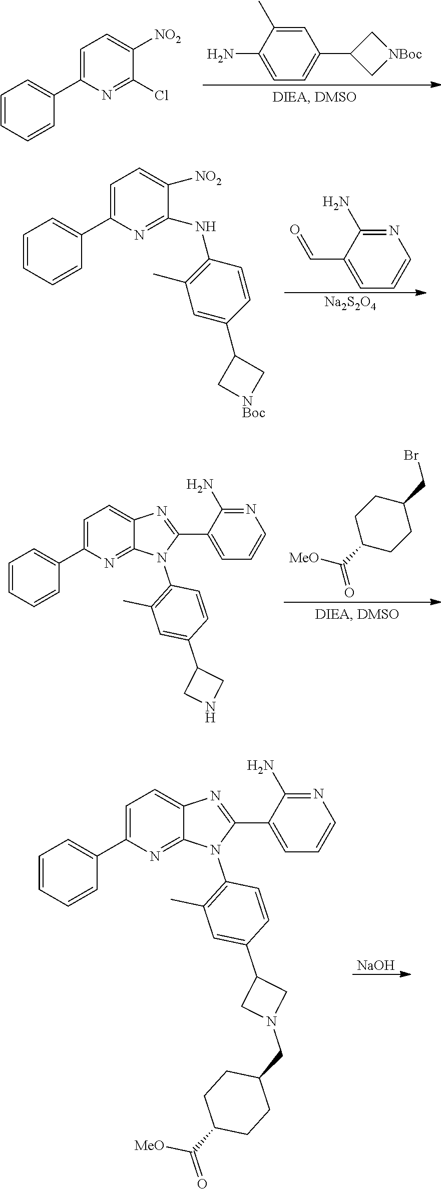

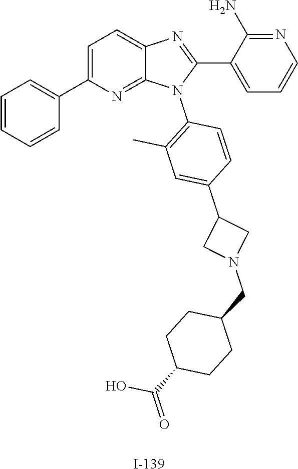 [SMILES: Cc1cc(C2CN(C[C@H]3CC[C@H](C(=O)O)CC3)C2)ccc1-n1c(-c2cccnc2N)nc2ccc(-c3ccccc3)nc21 ; "I-139"]

Step 1. To a solution of tert-butyl 3-(4-amino-3-methyl-phenyl)azetidine-1-carboxylate (3.00 g, 11 4 mmol) in DMSO (30 mL), 2-chloro-3-nitro-6-phenyl-pyridine (2.68 g, 11.4 mmol) and DIEA (5.50 mL, 34.3 mmol) were added, under N₂ atmosphere, and the mixture was stirred at 90° C. for 6 hrs. The reaction was taken up in EtOAc (100 mL) and the organics were washed with water (50 mL) and saturated brine solution (50 mL×3). The organics were then separated and dried (Na₂SO₄), and subsequently concentrated in vacuo. The crude was then purified by flash column chromatography, eluting with 20% EtOAc in isohexane, to provide tert-butyl 3-[3-methyl-4-[(3-nitro-6-phenyl-2-pyridyl)amino]phenyl]azetidine-1-carboxylate (2.70 g, 51.27% yield) as a yellow oil. LCMS Rₜ=0.848 min in 1 min chromatography, purity 81.2%, MS ESI calcd. for 460.21 [M+H]⁺ 461.21, found 461.1.

Step 2. To a solution of tert-butyl 3-[3-methyl-4-[(3-nitro-6-phenyl-2-pyridyl)amino]phenyl]azetidine-1-carboxylate (600 mg, 1.29 mmol) in DMSO (5 mL) and methanol (1 mL), 2-aminopyridine-3-carbaldehyde (316 mg, 2.58 mmol) and Na₂S₂O₄ (1.35 g, 7.75 mmol) were added at 25° C. and the mixture was stirred at 100° C. for 10 hrs. EtOAc (100 mL) was added and the organics washed with water (50 mL) and then a saturated brine solution (50 mL×3). The organics were then separated, dried (Na₂SO₄) and subsequently concentrated in vacuo. The crude was then purified by flash column chromatography, eluting with 100% EtOAc in isohexane, to provide the crude material. The residue was purified by prep-HPLC (Column:Welch Xtimate C18 40*200 mm 7 um; mobile phase: [water(FA)-ACN]; B %: 8%-48%, 25 min) to give 3-(3-(4-(azetidin-3-yl)-2-methylphenyl)-5-phenyl-3H-imidazo[4,5-b]pyridin-2-yl)pyridin-2-amine (450 mg, 25.94% yield) as a yellow solid. ¹H NMR (DMSO-d₆) δ ppm 8.30 (d, J=8.4 Hz, 1H), 8.24 (s, 1H), 7.95-8.02 (m, 4H), 7.54 (s, 1H), 7.42-7.49 (m, 4H), 7.27 (s, 2H), 7.06 (dd, J=7.6, 1.6 Hz, 1H), 6.36 (dd, J=7.6, 4.8 Hz, 1H), 4.12-4.29 (m, 6H), 2.01 (s, 3H).

Step 3. To a solution of 3-[3-[4-(azetidin-3-yl)-2-methyl-phenyl]-5-phenyl-imidazo[4,5-b]pyridin-2-yl]pyridin-2-amine (400 mg, 0.925 mmol) in DMSO (2 mL), DIEA (0.43 mL, 2.77 mmol) and trans-methyl 4-(bromomethyl)cyclohexanecarboxylate (261 mg, 111 mmol) were added under a N₂ atmosphere. The reaction mixture was degassed and purged with N₂ (×3), and the reaction was stirred at 80° C. for 6 hrs. EtOAc (50 mL) was added to the reaction mixture and the organics were washed with water (20 mL), and then a saturated brine solution (20 mL×3). The organics were then separated, dried (Na₂SO₄), and subsequently concentrated in vacuo to provide trans-methyl 4-[[3-[4-[2-(2-amino-3-pyridyl)-5-phenyl-imidazo[4,5-b]pyridin-3-yl]-3-methyl-phenyl]azetidin-1-yl]methyl]cyclohexanecarboxylate (160 mg, 22.12% yield) as a yellow oil. ¹H NMR (DMSO-d₆) δ ppm 8.27 (d, J=8.4 Hz, 1H), 7.95 (d, J=8.4 Hz, 4H), 7.27-7.48 (m, 8H), 7.03 (dd, J=7.6, 1.6 Hz, 1H), 6.34 (dd, J=7.6, 4.8 Hz, 1H), 3.59-3.70 (m, 4H), 3.12 (s, 3H), 2.27-2.32 (m, 2H), 1.96 (s, 3H), 1.89 (s, 3H), 1.76-1.87 (m, 3H), 1.20-1.32 (m, 3H), 0.88-0.95 (m, 2H).

Step 4. To a solution of trans-methyl 4-[[3-[4-[2-(2-amino-3-pyridyl)-5-phenyl-imidazo[4,5-b]pyridin-3-yl]-3-methyl-phenyl]azetidin-1-yl]methyl]cyclohexanecarboxylate (160 mg, 0.273 mmol) in THF (2 mL) and water (2 mL), NaOH (33 mg, 0.818 mmol) was added and the mixture was stirred at 25° C. for 16 hrs. The crude product was purified by prep-HPLC(column: Welch Xtimate C18 200*40 mm*7 μm; mobile phase: [water (NH₄HCO₃)-ACN]; B %: 10%-50%, 25 min) to provide trans-4-[[3-[4-[2-(2-amino-3-pyridyl)-5-phenyl-imidazo[4,5-b]pyridin-3-yl]-3-methyl-phenyl]azetidin-1-yl]methyl]cyclohexanecarboxylic acid I-139 (69 mg, 44.18% yield) as a yellow solid. ¹H NMR (DMSO-d₆) δ ppm 8.28 (d, J=8.4 Hz, 1H), 7.89-8.04 (m, 4H), 7.22-7.50 (m, 8H), 7.04 (dd, J=7.6, 1.6 Hz, 1H), 6.35 (dd, J=7.6, 4.8 Hz, 1H), 3.60-3.71 (m, 3H), 3.08-3 13 (m, 2H), 2.29 (d, J=6.8 Hz, 2H), 2.10 (tt, J=12.0, 3.2 Hz, 1H), 1.97 (s, 3H), 1.76-1.91 (m, 4H), 1.17-1.36 (m, 3H), 0.82-0.98 (m, 2H). HPLC Rₜ=2.088 in 8 min chromatography, ACE Excel 3 C18 4.6*100 mm, purity 100%. LCMS Rₜ=1.536 min in 4 min chromatography, purity 99.94%, MS ESI calcd. for 572.29 [M+H]⁺ 573.29, found 573.3.

### Example 131. Synthesis of 4-((3-(4-(2-(2-aminopyridin-3-yl)-5-phenyl-3H-imidazo[4,5-b]pyridin-3-yl)phenyl)azetidin-1-yl)methyl)cyclohexane-1-carboxamide (Compound I-140)

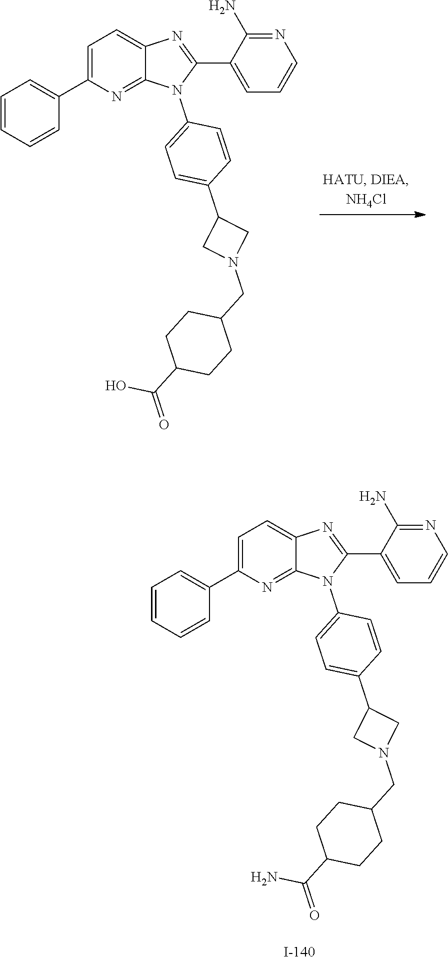 [SMILES: Nc1ncccc1-c1nc2ccc(-c3ccccc3)nc2n1-c1ccc(C2CN(CC3CCC(C(=O)O)CC3)C2)cc1 ; NC(=O)C1CCC(CN2CC(c3ccc(-n4c(-c5cccnc5N)nc5ccc(-c6ccccc6)nc54)cc3)C2)CC1]

Step 1. To a stirred mixture of 4-((3-(4-(2-(2-aminopyridin-3-yl)-5-phenyl-3H-imidazo[4,5-b]pyridin-3-yl)phenyl)azetidin-1-yl)methyl)cyclohexane-1-carboxylic acid (170 mg, 0.304 mmol) and HATU (174 mg, 0.456 mmol) in DMSO (5 mL) was NH₄Cl (65 mg, 1.22 mmol). The mixture was stirred at 30° C. for 16 hrs at which point water (50 mL) was added. The water phase was extracted with EtOAc (3×50 mL), the organic phase was collected then washed with brine (20 mL) and dried over Na₂SO₄ then concentrated. The residue was purified by prep-HPLC (Xtimate C18 150*40 mm*5 μm, water (FA)-ACN) to give 4-((3-(4-(2-(2-aminopyridin-3-yl)-5-phenyl-3H-imidazo[4,5-b]pyridin-3-yl)phenyl)azetidin-1-yl))methyl)cyclohexane-1-carboxamide I-140 (36.0 mg, 0.0616 mmol, 20% yield) as a yellow solid. ¹H NMR (400 MHz, DMSO-d₆) δ=8.27 (d, J=8.4 Hz, 1H), 8.17 (s, 2H), 8.06-7.96 (m, 4H), 7.57-7.51 (m, 2H), 7.50-7.43 (m, 4H), 7.42-7.36 (m, 1H), 7.20 (dd, J=1.6, 7.6 Hz, 1H), 7.16 (br s, 1H), 6 94 (s, 2H), 6.63 (br s, 1H), 6.42 (dd, J=4.8, 7.6 Hz, 1H), 3.75 (s, 3H), 3.27 (br s, 2H), 2.41 (d, J=6.4 Hz, 2H), 2.05-1.95 (m, 1H), 1.83-1.69 (m, 4H), 1.36-1.23 (m, 3H), 0 96-0.81 (m, 2H). HPLC Rₜ=3.498 min in 8 min chromatography, purity 96 2% LCMS Rₜ=3.929 min in 7 min chromatography, purity 96.5%, MS ESI calcd. For 557.29 [M+H]⁺ 558.29, found 558.1.

### Example 132. Synthesis of (1r,4r)-N1-(5-(2-(2-aminopyridin-3-yl)-5-phenyl-3H-imidazo[4,5-b]pyridin-3-yl)-6-methylpyridin-2-yl)-N4-methylcyclohexane-1,4-dicarboxamide (Compound I-141)

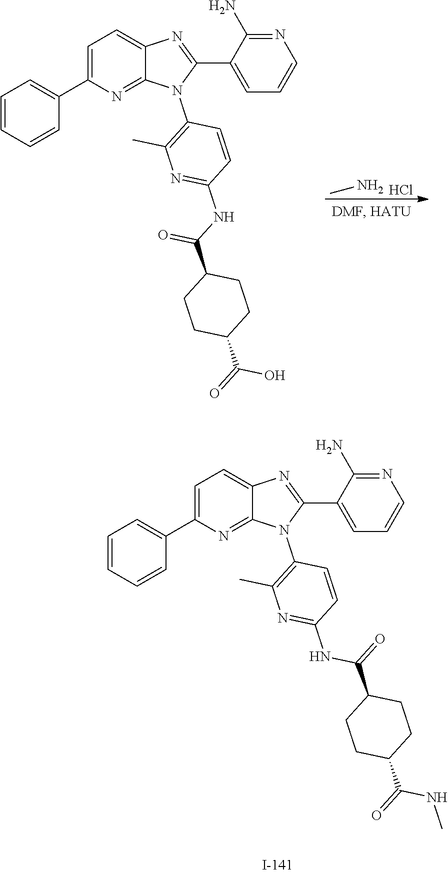

Step 1. To a solution of trans-4-((5-(2-(2-aminopyridin-3-yl)-5-phenyl-3H-imidazo[4,5-b]pyridin-3-yl)-6-methylpyridin-2-yl)carbamoyl)cyclohexane-1-carboxylic acid (150 mg, 0.274 mmol) and methanamine (43.0 mg, 1.37 mmol) in DMF (5 mL) was added DIEA (106 mg, 0.822 mmol) and HATU (208 mg). The mixture was stirred at 35° C. for 2 hrs. The reaction mixture was concentrated directly and the residue was purified by prep-HPLC (Column: Welch Xtimate C18 150*30 mm*5 um; mobile phase: [water (FA)-ACN]; B %: 0%-35%; 25 min) to afford trans-N1-(5-(2-(2-aminopyridin-3-yl)-5-phenyl-3H-imidazo[4,5-b]pyridin-3-yl)-6-methylpyridin-2-yl)-N4-methylcyclohexane-1,4-dicarboxamide I-141 (20.7 mg, 0.0356 mmol, 13% yield) as a yellow solid. ¹H NMR (DMSO-d₆, 400 MHz,) δ=10.68 (s, 1H), 8.30 (d, J=8.4 Hz, 1H), 8.12 (d, J=8.8 Hz, 1H), 8.04-7.97 (m, 4H), 786 (d, J=8.4 Hz, 1H), 7.68 (q, J=4.4 Hz, 1H), 7.49-7.37 (m, 3H), 7.25-7.11 (m, 3H), 6.46 (dd, J=4 8, 7.6 Hz, 1H), 2.56 (d, J=4.4 Hz, 4H), 2.15-2.04 (m, 4H), 1.93-1.74 (m, 4H), 1.49-1.33 (m, 4H). HPLC Rₜ=3.389 min in 8 min chromatography, purity 96%. LCMS Rt=1.250 min in 2.5 min chromatography, purity 99.3%, MS ESI calcd. for 560.26, [M+H]⁺ 561.26, found 561.3.

### Example 133. Synthesis of trans-N-(5-(2-(2-aminopyridin-3-yl)-5-phenyl-3H-imidazo[4,5-b]pyridin-3-yl)-6-methylpyridin-2-yl)cyclohexane-1,4-dicarboxamide (Compound I-142)

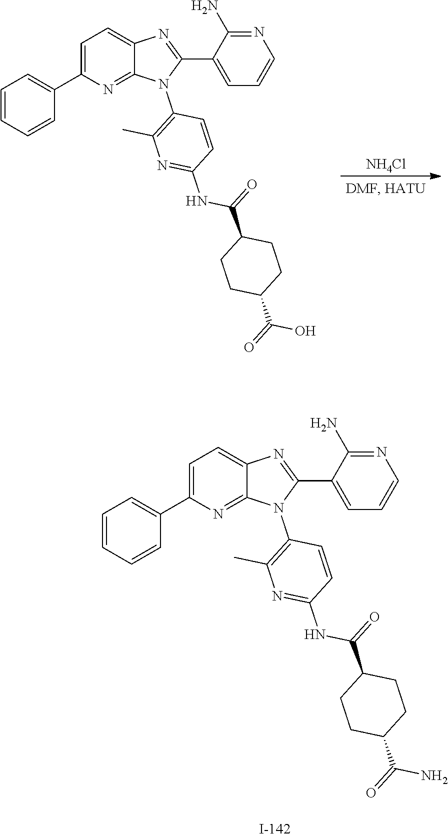 [SMILES: Cc1nc(NC(=O)[C@H]2CC[C@H](C(=O)O)CC2)ccc1-n1c(-c2cccnc2N)nc2ccc(-c3ccccc3)nc21 ; "NH4Cl" ; Cc1nc(NC(=O)[C@H]2CC[C@H](C(N)=O)CC2)ccc1-n1c(-c2cccnc2N)nc2ccc(-c3ccccc3)nc21]

Step 1. To a solution of trans-4-((5-(2-(2-aminopyridin-3-yl)-5-phenyl-3H-imidazo[4,5-b]pyridin-3-yl)-6-methylpyridin-2-yl)carbamoyl)cyclohexane-1-carboxylic acid (150 mg, 0.274 mmol) and NH₄Cl (73.0 mg, 1.37 mmol) in DMF (5 mL) was added DIEA (106 mg, 0.822 mmol) and HATU (208 mg). The mixture was stirred at 35° C. for 2 hrs. The reaction mixture was concentrated directly. The residue was purified by prep-HPLC (Column: Welch Xtimate C18 150*30 mm*5 um; mobile phase. [water (FA)-ACN]; B %: 0%-35%; 25 min) to afford trans-N-(5-(2-(2-aminopyridin-3-yl)-5-phenyl-3H-imidazo[4,5-b]pyridin-3-yl)-6-methylpyridin-2-yl)cyclohexane-1,4-dicarboxamide I-142 (24 3 mg, 0.0435 mmol, 15.9% yield) as a yellow solid. 1H NMR (DMSO-d₆, 400 MHz,) δ=10.68 (s, 1H), 8.30 (d, J=8.4 Hz, 1H), 8.12 (d, J=8.8 Hz, 1H), 8.05-7.97 (m, 4H), 7.86 (d, J=8.8 Hz, 1H), 7.49-7.37 (m, 3H), 7.25-7.13 (m, 4H), 6.68 (s, 1H), 6.47 (dd, J=4.8, 7.6 Hz, 1H), 2.57-2.51 (m, 1H), 2.15-2.03 (m, 4H), 1.93-1.78 (m, 4H), 1.50-1.31 (m, 4H). HPLC Rt=3.244 min in 8 min chromatography, purity 98%. LCMS Rₜ=1.266 min in 2.5 min chromatography, purity 98%, MS ESI calcd. for 546 25, [M+H]⁺ 547.25, found 547.3.

### Synthesis of Additional Intermediates for Table 2 Compounds

### Synthesis of methyl 4-(hydroxymethyl)-3,3-dimethylcyclohexane-1-carboxylate

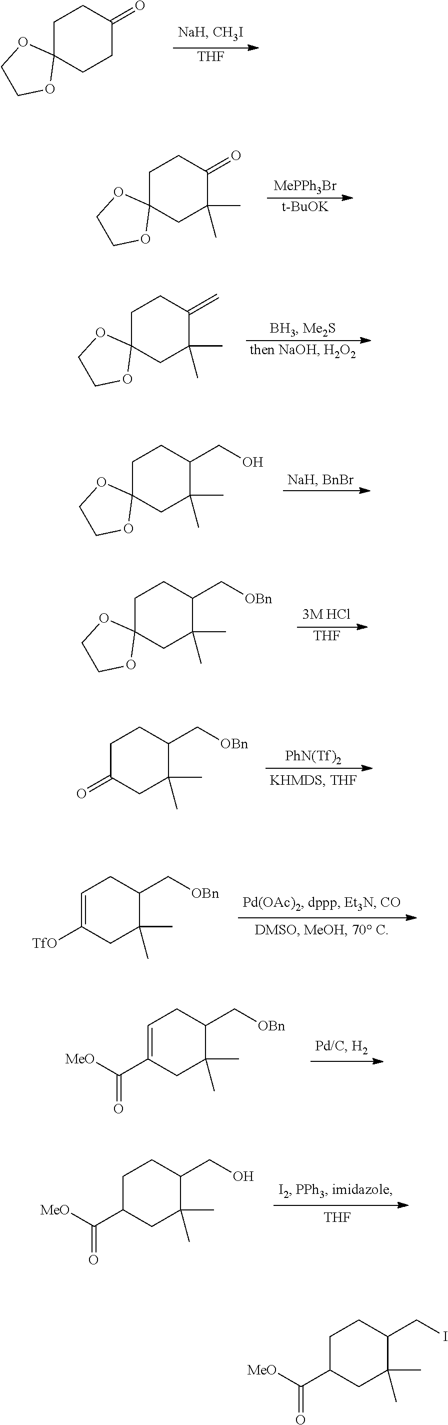

Step 1. To a solution of NaH (10.2 g, 256 mmol, 60%) in THF (120 mL) was added a solution of 1,4-dioxaspiro[4.5]decan-8-one (20.0 g, 128 mmol) in THF (80 mL) at 0° C. under N₂. The mixture was stirred at 25° C. for 1 hr. To the reaction mixture was added CH₃I (20.0 mL, 320 mmol) slowly at 25° C. then stirred for 16 hrs under N₂. The reaction mixture was quenched by addition of sat aq. NH₄Cl (200 mL) and extracted with EtOAc (3×150 mL). The organic layer was dried over Na₂SO₄ and concentrated. The crude was purified by flash column (eluent of 0~10% EtOAc in PE) to give 7,7-dimethyl-1,4-dioxaspiro[4.5]decan-8-one (12.2 g, 52% yield) as colorless oil. ¹H NMR (400 MHz, DMSO-d₆) δ=3.92 (s, 4H), 2 47-2.40 (m, 2H), 1 93 (t, J=6.8 Hz, 2H), 1.84 (s, 2H), 1.07 (s, 6H).

Step 2. To a solution of methyl(triphenyl)phosphonium bromide (71.0 g, 199 mmol) in THF (360 mL) was added t-BuOK (22.3 g, 199 mmol). The mixture was stirred at 25° C. for 1 hr under N₂. Then to the reaction mixture was added a solution of 7,7-dimethyl-1,4-dioxaspiro[4.5]decan-8-one (12.2 g, 66.2 mmol) in THF (30 mL) at 10° C. The resulting mixture was then stirred at 50° C. for 3 hrs. The reaction mixture was quenched by addition of sat aq. NH₄Cl (300 mL) slowly at 20° C., then extracted with EtOAc (3×200 mL). The organic layer was dried over Na₂SO₄ and concentrated. The crude was purified by flash column (0-10% of EtOAc in PE) to give 7,7-dimethyl-8-methylene-1,4-dioxaspiro[4.5]decane (12.0 g, 99% yield) as yellow oil. ¹H NMR (400 MHz, DMSO-d₆) δ=4.75-4.59 (m, 2H), 3.85 (s, 4H), 2.33-2.24 (m, 2H), 1.63-1.55 (m, 2H), 1.49 (s, 2H), 1.08 (s, 6H).

Step 3. To a solution of 7,7-dimethyl-8-methylene-1,4-dioxaspiro[4.5]decane (12.0 g, 65.8 mmol) in THF (120 mL) was added IOM BH₃·Me₂S (2.28 g, 165 mmol, 16.5 mL). The mixture was stirred at 0° C. for 3 hrs under N₂. Ethanol (180 mL) was cautiously added dropwise followed by 3M NaOH (13.2 g, 329 mmol, 120 mL). After stirring at 0° C. for 1 h, 30% H₂O₂ (66.0 mL, 658 mmol) was added at 0° C. under N₂. After stirring at 25° C. for another 16 hrs under N₂. The mixture cooled to 20-25° C., then was cooled down to 5-10° C. with ice-water bath. Saturated Na₂SO₃ (300 mL) was transferred to an addition funnel, then was added to the reaction mixture dropwise at 0-5° C. with stirring within 20 mins, then was stirred at this temperature for 1 hr. After checking by starch potassium iodide test paper, the resulting mixture was extracted with EtOAc (200 mL×3). The organic layer was dried over Na₂SO₄ and concentrated to give a crude. The crude was purified by flash column (eluent of 0~60% EtOAc in PE) to give (7,7-dimethyl-1,4-dioxaspiro[4 5]decan-8-yl)methanol (11.1 g, 84% yield) as colorless oil. ¹H NMR (400 MHz, DMSO-d₆) 3=4.28 (dd, J=4.8, 5.6 Hz, 1H), 3.89-3.73 (m, 4H), 3.64-3.55 (m, 1H), 3.11-3.00 (m, 1H), 1.84-1.75 (m, 1H), 1.72-1.64 (m, 1H), 1.46-1.24 (m, 4H), 1.23-1.14 (m, 1H), 0.93 (s, 3H), 0.81 (s, 3H).

Step 4. To the solution of NaH (4.43 g, 111 mmol, 60%) in THF (110 mL) was added a solution of (7,7-dimethyl-1,4-dioxaspiro[4.5]decan-8-yl)methanol (11.1 g, 55.4 mmol) in THF (20 mL). The mixture was stirred at 25° C. for 1 hr. Then to mixture was added BnBr (16.0 mL, 139 mmol). After stirring at 50° C. for 16 hrs. The reaction mixture was added H₂O (100 mL) and extracted with EtOAc (100 mL×3). The organic layer was dried over Na₂SO₄, filtered and concentrated. The crude was purified by flash column (eluent of 0~10% EtOAc in PE) to give 8-((benzyloxy)methyl)-7,7-dimethyl-1,4-dioxaspiro[4.5]decane (14.3 g, 89% yield) as yellow oil. ¹H NMR (400 MHz, DMSO-d₆) δ=7.42-7.19 (m, 5H), 4.44 (q, J=12.0 Hz, 2H), 3 90-3.82 (m, 2H), 3.80-3.72 (m, 2H), 3.57 (dd, J=3.6, 9.2 Hz, 1H), 3.16 (t, J=8.8 Hz, 1H), 1.82-1.73 (m, 1H), 1.71-1.61 (m, 1H), 1.49-1.31 (m, 5H), 0.94 (s, 3H), 0.83 (s, 3H).

Step 5. To a solution of 8-((benzyloxy)methyl)-7,7-dimethyl-1,4-dioxaspiro[4.5]decane (14.6 g, 50.3 mmol) in THF (150 mL) was added 2M HCl (150 mL). The mixture was stirred at 25° C. for 16 hrs. The reaction was poured into water (100 mL), and extracted with EtOAc (200 mL*3). The organic phase was washed with brine (10 mL), dried with Na₂SO₄ and concentrated. The residue was purified by column (eluent of 0~10% EtOAc in PE) to give 4-((benzyloxy)methyl)-3,3-dimethylcyclohexan-1-one (12.0 g, 97% yield) as yellow oil. ¹H NMR (400 MHz, DMSO-d₆) δ=7.49-7.22 (m, 5H), 4.57-4.38 (m, 2H), 3 71-3 60 (m, 1H), 3.32-3.28 (m, 1H), 2.49-2.44 (m, 1H), 2.39-2.26 (m, 2H), 2.22-2.14 (m, 1H), 2.12-2.02 (m, 1H), 1.97-1.87 (m, 2H), 1.67-1.51 (m, 1H), 1.01 (d, J=3.2 Hz, 3H), 0.74 (d, J=2.8 Hz, 3H).

Step 6. To a solution of 4-(benzyloxymethyl)-3,3-dimethyl-cyclohexanone (12.0 g, 48.7 mmol) in THF (120 mL) was added KHMDS (63.3 mmol, 1M, 63.3 mL) at −78° C. under N₂. The mixture was stirred at −78° C. for 1 hr under N₂. To the mixture was added a solution of n-phenylbis(trifluoromethanesulfonimide (19.1 g, 53.6 mmol) in THF (100 mL). The mixture was stirred at 25° C. for 3 hrs. Sat. aq. NH₄Cl solution (100 mL) was added and the mixture was extracted with EtOAc (3×100 mL). The combined organic layers were dried over Na₂SO₄, filtered and concentrated. The crude was purified by column (eluent of 0~5% EtOAc in PE) to give [4-(benzyloxymethyl)-5,5-dimethyl-cyclohexen-1-yl]trifluoromethanesulfonate (14.0 g, 76% yield) as colorless oil. ¹HNMR (400 MHz, DMSO-d₆) δ=7.43-7.20 (m, 5H), 5.85 (s, 1H), 4.54-4.36 (m, 2H), 3.57 (dd, J1=4.4, 9.2 Hz, 1H), 3.26 (t, J=9.2 Hz, 1H), 2.42-2.28 (m, 1H), 2.21 (dd, J=2.4, 17.2 Hz, 1H), 2.15-1.95 (m, 2H), 1.73-1.61 (m, 1H), 1.00 (s, 3H), 0.88 (s, 3H).

Step 7. To a solution of 4-((benzyloxy)methyl)-5,5-dimethylcyclohex-1-en-1-yl trifluoromethanesulfonate (6.50 g, 17.2 mmol) in DMSO (30 mL) and methanol (50 mL) was added dppp (1.42 g, 3.44 mmol), Et₃N (7.20 mL, 51.5 mmol) and Pd(OAc)₂ (386 mg, 1.72 mmol). The mixture was stirred at 80° C. for 16 hrs under CO (50 psi). To the mixture was added water (50 mL). The mixture was extracted with EtOAc (3×100 mL). The organic phase was washed with brine (100 mL), dried with Na₂SO₄ and concentrated. The residue was purified by column (eluent of 0~8% EtOAc in PE) to give methyl 4-((benzyloxy)methyl)-5,5-dimethylcyclohex-1-ene-1-carboxylate (4.00 g, 81% yield) as colorless oil. ¹HNMR (400 MHz, DMSO-d₆) δ=7.36-7 15 (m, 5H), 6.85-6.77 (m, 1H), 4.49-4.29 (m, 2H), 3.57 (s, 3H), 3.52 (dd, J=4.4, 9.2 Hz, 1H), 3.16 (t, J=8.8 Hz, 1H), 2.37-2.28 (m, 1H), 2.01 (dd, J=2.8, 5.6, 20.0 Hz, 1H), 1.94-1.92 (m, 2H), 1.63-1.51 (m, 1H), 0.89 (s, 3H), 0.72 (s, 3H).

Step 8. To a solution of methyl 4-((benzyloxy)methyl)-5,5-dimethylcyclohex-1-ene-1-carboxylate (4.00 g, 13.9 mmol) in ethyl acetate (3 mL) was added wet Pd/C (0.5 g, 13.9 mmol, 10% purity) under argon. The reaction was degassed and purged with Ar for 3 times, then degassed and purged with H₂ for 3 times. The mixture was stirred under H₂ (50 Psi) for 16 hrs at 50° C. Upon completion the mixture was filtered under argon to afford methyl 4-(hydroxymethyl)-3,3-dimethylcyclohexane-1-carboxylate (2.00 g, 72% yield) as colorless oil. ¹HNMR (400 MHz, DMSO-d₆) δ=4.34-4.27 (m, 1H), 3.57 (s, 3H), 3.56-3.49 (m, 1H), 3.27 (td, J=5.2, 10.0 Hz, 1H), 2.50-2.38 (m, 1H), 1.71-1.47 (m, 4H), 1.40-1.32 (m, 1H), 1.30-1.21 (m, 1H), 0.96 (s, 3H), 0.84-0.66 (m, 3H).

Step 9. To a solution of methyl 4-(hydroxymethyl)-3,3-dimethylcyclohexane-1-carboxylate (500 mg, 2.50 mmol) in THF (6 mL) was added imidazole (476 mg, 6.99 mmol), PPh₃ (1.70 g, 6.49 mmol) and I₂ (1 65 g, 6.49 mmol) at 0° C. under N₂ atmosphere. The mixture was stirred at 25° C. for 16 hrs. Water (20 mL) was added to the residue. The resulting mixture was extracted with EtOAc (3×50 mL). The combined organic phase was washed with brine (20 mL), dried over anhydrous Na₂SO₄, filtered and concentrated. The residue was purified by flash silica gel chromatography eluent of 0 6% EtOAc in PE) to give methyl 4-(iodomethyl)-3,3-dimethylcyclohexane-1-carboxylate (700 mg, 90% yield) as colorless oil. ¹HNMR (400 MHz, DMSO-d₆) δ=3.56-3.50 (m, 4H), 3.09 (t, J=10.8 Hz, 1H), 2.42-2.31 (m, 1H), 1.73 (ddd, J=4.4, 9 2, 13.6 Hz, 1H), 1.62 (dt, J=4.8, 9.6 Hz, 1H), 1.53-1.39 (m, 4H), 1.34-1 28 (m, 1H), 0.96-0.92 (m, 3H), 0.77 (s, 3H).

### Synthesis of Benzyl 5-formylbicyclo[2.2.1]heptane-2-carboxylate

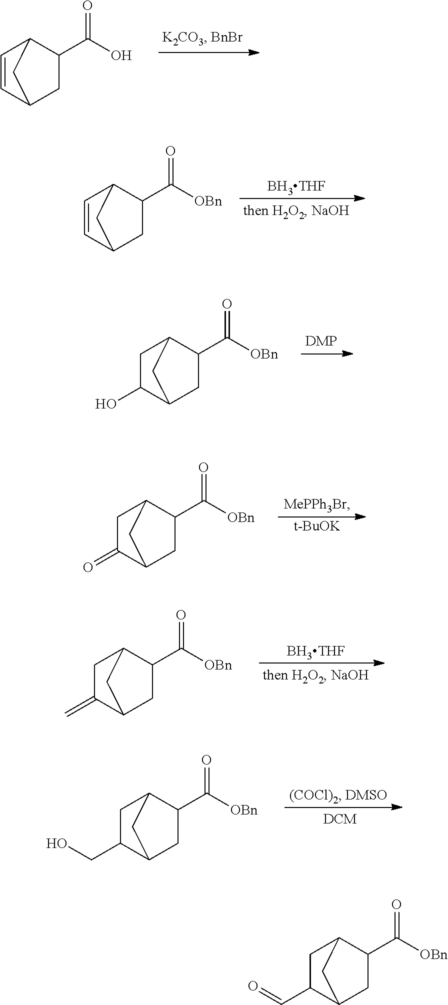

Step 1. To a solution of bicyclo[2.2.1]hept-5-ene-2-carboxylic acid (10 0 g, 72.4 mmol) in DMF (100 mL) was added K₂CO₃ (20.0 g, 145 mmol). BnBr (9.5 mL, 79.6 mmol). The mixture was stirred at 25° C. for 16 hrs. Water was added to the mixture. The mixture was extracted with EtOAc (3×200 mL). The organic phase was washed with brine (3×50 mL), dried with Na₂SO₄ and concentrated. The residue was purified by column (eluent of 0-1% EtOAc in PE) to give benzyl bicycle[2.2.1]hept-5-ene-2-carboxylate (16.0 g, 70.1 mmol, 97% yield) as colorless oil. ¹H NMR (400 MHz, DMSO-d₆) δ=(400 MHz, CHLOROFORM-d) δ=7.44-7.27 (m, 5H), 6.19 (dd, J=3.2, 5.6 Hz, 1H), 5.88 (dd, J=2.8, 5 6 Hz, 1H), 5.15-5.02 (m, 2H), 3.24 (s, 1H), 3.04-2.98 (m, 1H), 2.92 (s, 1H), 1.99-1.85 (m, 1H), 1.52-1.34 (m, 2H), 1.28 (d, J=8.0 Hz, 1H).

Step 2. To the solution of benzyl bicyclo[2.2.1]hept-5-ene-2-carboxylate (18.0 g, 78.8 mmol) in THF (150 mL) was added BH₃·Me₂S (436 mg, 31.5 mmol, 3.1 mL) then stirred at 25° C. for 5 min under N₂. To the mixture was added THF (50 mL), the whole was stirred for 4 hrs. To the mixture by ice-bath were successively added dioxane (50 mL), 3N NaOH (15 mL), 30% H₂02 (9.7 mL, 315 mmol, 10.7 g). The mixture was stirred at 0° C. for 20 min. The mixture cooled to 20-25° C., then was cooled down to 0-5° C. with ice-water bath. Saturated Na₂SO₃ (300 mL) was transferred to an addition funnel, then was added to the reaction mixture dropwise at 0-5° C. with stirring within 20 min, then was stirred at this temperature for 1 hr. After checking by starch potassium iodide test paper, the resulting mixture was extracted with EtOAc (3×200 mL). The organic layer was dried over Na₂SO₄ and concentrated in vacuum to give a crude. The residue was purified by column (eluent of 0-23% EtOAc in PE) to give benzyl 5-hydroxybicyclo[2.2.1]heptane-2-carboxylate (6.00 g, 24.4 mmol, 31% yield) as colorless oil. ¹H NMR (400 MHz, CHLOROFORM-d) δ=7.40-7.28 (m, 5H), 5.14-5.05 (m, 2H), 3.84 (d, J=6.8 Hz, 1H), 2.79-2.66 (m, 1H), 2.63-2.52 (m, 1H), 2.29-2.16 (m, 1H), 1.77-1.69 (m, 2H), 1.55-1.48 (m, 2H), 1.32 (dd, J=1.2, 11.2 Hz, 1H), 1.18-1.16 (m, 1H), 1.28-1.15 (m, 1H).

Step 3. To the mixture of benzyl 5-hydroxybicyclo[2.2.1]heptane-2-carboxylate (6.00 g, 24.4 mmol) in DCM (60 mL) was added DMP (11 8 g, 60.9 mmol) at 0° C. The mixture was stirred at 25° C. for 3 hrs. Water (50 mL) was added to the mixture. The mixture was extracted with DCM (3×50 mL). The organic phase was washed with brine (30 mL), dried with Na₂SO₄ and concentrated. The mixture was purified by column (eluent of 0 20% EtOAc in DCM) to give benzyl 5-oxobicyclo[2.2.1]heptane-2-carboxylate (3.60 g, 14.7 mmol, 61% yield) as colorless oil. ¹H NMR (400 MHz, CHLOROFORM-d) δ=7.42-7.28 (m, 5H), 5.20-5.06 (m, 2H), 3.14-3.03 (m, 1H), 2.98 (d, J=0.8 Hz, 1H), 2.64 (d, J=4.8 Hz, 1H), 2.08-1.97 (m, 4H), 1.86-1.77 (m, 1H), 1.76-1 69 (m, 1H).

Step 4. To the solution of methyl(triphenyl)phosphanium;bromide (14.9 g, 41.8 mmol) in THF (40 mL) was added t-BuOK (42 mL, 41.8 mmol, 1M) then stirred at 25° C. for 1 hr under N₂. Then to the reaction mixture was added a solution of benzyl 5-oxobicyclo[2.2.1]heptane-2-carboxylate (3.40 g, 13.9 mmol) in THE (10 mL) at 10° C. Then stirred at 20° C. for 1 hr. At 0° C., the reaction mixture was quenched by the addition of saturated aqueous ammonium chloride (50 mL) slowly then extracted with EtOAc (3×100 mL). The organic layer was dried over Na₂SO₄ and concentrated to give a crude. The crude was purified by flash column (eluent of 0-10% EtOAc in PE). Benzyl 5-methylenebicyclo[2.2.1]heptane-2-carboxylate (1.85 g, 7.63 mmol, 55% yield) was obtained as colorless oil. ¹HNMR (400 MHz, DMSO-d₆) δ=7.44-7.28 (m, 5H), 5.16-5 02 (m, 2H), 4.84 (br d, J=18.0 Hz, 1H), 4.62-4.46 (m, 1H), 2.70 (br dd, J=3.6, 12.4 Hz, 1H), 2.65-2.55 (m, 1H), 2.20-2.00 (m, 1H), 1.95-1.78 (m, 2H), 1.67-1.59 (m, 1H), 1.57-1.44 (m, 1H), 1.41 (dd, J=1.6, 9.6 Hz, 1H), 1.34-1.23 (m, 1H).

Step 5. To the solution of benzyl 5-methylenebicyclo[2.2.1]heptane-2-carboxylate (1600 mg, 6.60 mmol) in THF (15 mL) was added BH₃·Me₂S (37.0 mg, 2.64 mmol) (0.26 mL) then stirred at 25° C. for 4 hrs under N₂. To the mixture by ice-bath were successively added dioxane (3 mL), 3N NaOH (1 mL), 30% H₂02 (0.81 mL, 26.4 mmol)(898 mg) at 0° C. The mixture was stirred at 0° C. for 20 min. The mixture cooled to 20-25° C., then was cooled down to 0-5° C. with ice-water bath. Saturated Na₂SO₃ (300 mL) was transferred to an addition funnel, then was added to the reaction mixture dropwise at 0-5° C. with stirring within 20 min, then was stirred at this temperature for 1 h. After checking by starch potassium iodide test paper, the resulting mixture was extracted with EtOAc (3×100 mL). The organic layer was dried over Na₂SO₄ and concentrated in vacuum to give a crude. The residue was purified by column (eluent of 0~10% EtOAc in PE) to give benzyl 5-(hydroxymethyl) bicycle[2.2.1]heptane-2-carboxylate (1200 mg, 4.61 mmol, 70% yield). ¹HNMR (400 MHz, DMSO-d₆) δ=7.45-7.27 (m, 5H), 5.08 (d, J=11.2 Hz, 2H), 4.40-4.27 (m, 1H), 3.37 (dd, J=5.2, 7.4 Hz, 1H), 3.30-3.03 (m, 1H), 2.47-2.34 (m, 1H), 2.32-2.19 (m, 1H), 2.02-1.71 (m, 2H), 1.68-1.55 (m, 1H), 1.54-1.31 (m, 3H), 1.29-1.14 (m, 1H), 0.70-0.54 (m, 1H).

Step 6. To a solution of (COCl)₂ (0.10 mL, 1.15 mmol) in DCM (10 mL) was added a solution of DMSO (0 10 mL, 1.54 mmol) in DCM (0.5 mL) at −75° C. under N₂. The mixture was stirred at −75° C. for 10 min. To the mixture was added benzyl 5-(hydroxymethyl) bicycle [2.2.1]heptane-2-carboxylate (200 mg, 0.768 mmol) in DCM (2 mL) at −75° C. The mixture was stirred at −75° C. for 0.5 hr. TEA (0.35 mL, 2.54 mmol) was added to the mixture at −75° C. The mixture was stirred at −75° C. for 30 min. The mixture was stirred at 25° C. for 30 min. To the mixture was added water (20 mL). The water phase was extracted with DCM (3×20 mL). Benzyl 5-formylbicyclo[2.2.1]heptane-2-carboxylate (180 mg, 0.697 mmol, 91% yield) was obtained as colorless oil. ¹HNMR (400 MHz, DMSO-d₆) δ=9.75-9.52 (m, 1H), 7.38-7.32 (m, 5H), 5.11-5.05 (m, 2H), 2.97-2.77 (m, 1H), 2.68 (t, J=10.8 Hz, 1H), 2.61-2.53 (m, 1H), 2.46-2.32 (m, 1H), 1.87-1.79 (m, 1H), 1.64 (d, J=9.2 Hz, 1H), 1.58-1.49 (m, 1H), 1.42-1.35 (m, 1H), 1.27-1.07 (m, 2H).

### Synthesis of methyl 5-formyl-1,2,3,3a,4,5,6,6a-octahydropentalene-2-carboxylate

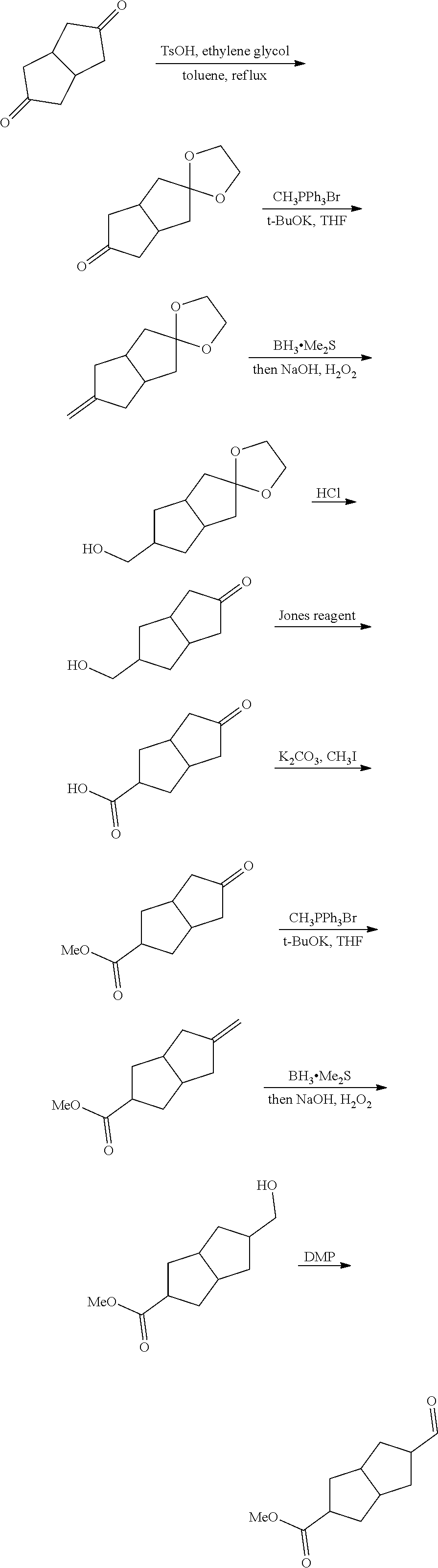

Step 1. To a solution of 1,3,3a,4,6,6a-hexahydropentalene-2,5-dione (30.0 g, 217 mmol) and ethylene glycol (13 mL, 239 mmol) in toluene (300 mL) was added TsOH (1.87 g, 10.9 mmol). After stirring at 115° C. under N₂ for 6 hrs, the reaction was cooled to r.t., poured into NaHCO₃ (500 mL) and extracted with EtOAc (3×300 mL). The combined organic was washed brine (1000 mL), dried over Na₂SO₄, filtered and concentrated. The residue was purified by flash column (20-25% of EtOAc in PE) to give spiro[1,3,3a,4,6,6a-hexahydropentalene-5,2′-1,3-dioxolane]-2-one (17.0 g, 93.3 mmol, 43% yield) as colorless oil. ¹H NMR (DMSO-d₆, 400 MHz) δ_{H}=3.81 (s, 4H), 2.81-2.67 (m, 2H), 2.47-2.36 (m, 2H), 2.13-1.97 (m, 4H), 1.68-1.57 (m, 2H).

Step 2. To a suspension of CH₃PPh₃Br (7.84 g, 22.0 mmol) in THF (50 mL) was added t-BuOK (2.46 g, 22.0 mmol). After stirring at 20° C. under N₂ for 1 hr, spiro[1,3,3a,4,6,6a-hexahydropentalene-5,2′-1,3-dioxolane]-2-one (2.00 g, 11.0 mmol) in THF (50 mL) was added, the reaction was stirred at 50 C under N₂ for 2 hr. The reaction was poured into H₂O (50 mL) and extracted with EtOAc (3×50 mL). The combined organic phase was washed with brine (150 mL), dried over Na₂SO₄, filtered and concentrated. The residue was purified by flash column (10% of EtOAc in PE) to give 5-methylenespiro[1,3,3a,4,6,6a-hexahydropentalene-2,2′-1,3-dioxolane](1.60 g, 8.88 mmol, 81% yield) as colorless oil. ¹H NMR (400 MHz, CHLOROFORM-d) δ_{H}=4.85-4.75 (m, 21), 3.93-3.84 (m, 4H), 2.66-2.42 (m, 4H), 2.12-1.94 (m, 4H), 1.62-1.51 (m, 2H).

Step 3. To the solution of 5-methylenespiro[1,3,3a,4,6,6a-hexahydropentalene-2,2′-1,3-dioxolane](1.60 g, 8.88 mmol) in THE (15 mL) was added BH₃·Me₂S (246 mg, 17.8 mmol, 1.78 mL) slowly at 0° C. under N₂. After stirring at 0° C. under N₂ for 4 hrs. Ethanol (10 mL) was added dropwise follow by NaOH (1.42 g, 35.5 mmol) (3 M, 11 mL) and stirred at 0° C. under N₂ for 0.5 hr. Then H₂O₂ (1.7 mL, 17.8 mmol) was added slowly at 0° C. under N₂ and the reaction was stirred at 20° C. under N₂ for 1 hr. The mixture cooled down to 0-5° C., Na₂S₂O₃ (30 mL, sat.) was added to the reaction mixture dropwise at 0-5° C. The mixture was stirred at this temperature for 1 hr and extracted with EtOAc (3×50 mL). The combined organic phase was dried over Na₂SO₄, filtered and concentrated. The residue was purified by column (50% of EtOAc in PE) to give spiro[1,3-dioxolane-2,5′-2,3,3a,4,6,6a-hexahydro-IH-pentalene]-2′-ylmethanol (1.30 g, 6.56 mmol) as colorless oil. ¹H NMR (400 MHz, CHLOROFORM-d) δ_{H}=3.93-3.84 (m, 4H), 3.74 (s, 1H), 3.64-3.55 (m, 2H), 2.63-2.43 (m, 2H), 2.11-1.93 (m, 5H), 1.64-1.55 (m, 2H), 1.20-1.02 (m, 2H).

Step 4. To a solution of spiro[1,3-dioxolane-2,5′-2,3,3a,4,6,6a-hexahydro-1H-pentalene]-2′-ylmethanol (1.30 g, 6.56 mmol) in acetone (10 mL) was added 3 M HCl (5.0 mL). After stirring at 20° C. for 16 hrs, the reaction was extracted with EtOAc (3×10 mL). The combined organic phase was brine (30 mL), dried over Na₂SO₄, filtered and concentrated to give 5-(hydroxymethyl)-3,3a,4,5,6,6a-hexahydro-1H-pentalen-2-one (1.00 g, 6.48 mmol) as colorless oil. ¹H NMR (400 MHz, CHLOROFORM-d) δ_{H}=3.65-3.55 (m, 2H), 2.85-2.68 (m, 2H), 2.55-2.44 (m, 2H), 2.39-2.13 (m, 3H), 2 11-2.03 (m, 2H), 1.79-1.65 (m, 1H), 1.19-1.06 (m, 2H).

Step 5. To a solution of 5-(hydroxymethyl)-3,3a,4,5,6,6a-hexahydro-1H-pentalen-2-one (1.00 g, 6.48 mmol) in Acetone (10 mL) was added slowly Jones reagent at 0° C. under N₂. After stirring at 20° C. under N₂ for 5 hrs, the reaction was quenched with 2-propanol (20 mL) at 0° C. under N₂ and diluted with water (20 mL) and extracted with DCM (3×50 mL). The combined organic phase was dried over Na₂SO₄, filtered and concentrated to give 5-oxo-2,3,3a,4,6,6a-hexahydro-1H-pentalene-2-carboxylic acid (620 mg, 3.69 mmol, 57% yield) as colorless oil. ¹H NMR (400 MHz, CHLOROFORM-d) δ_{H}=3.12-2.68 (m, 3H), 2.58-2.45 (m, 2H), 2.41-2.26 (m, 2H), 2.25-2.09 (m, 2H), 1.83-1.63 (m, 2H).

Step 6. To a solution of 5-oxo-2,3,3a,4,6,6a-hexahydro-1H-pentalene-2-carboxylic acid (620 mg, 3.69 mmol) in DMSO (10 mL) was added K₂CO₃ (1.53 g, 11.1 mmol) and Mel (0.34 mL, 5.53 mmol). After stirring at 20° C. under N₂ for 3 hrs, the reaction was poured into water (10 mL) and extracted with EtOAc (3×10 mL). The combined organic phase was washed brine (30 mL*3), dried over Na₂SO₄, filtered and concentrated. The residue was purified by flash column (15% of EtOAc in PE) to give methyl 5-oxo-2,3,3a,4,6,6a-hexahydro-IH-pentalene-2-carboxylate (420 mg, 2.30 mmol) as colorless oil. ¹H NMR (400 MHz, CHLOROFORM-d) δ1=3.68 (s, 3H), 2.97-2.85 (m, 1H), 2.82-2.66 (m, 2H), 2.57-2.42 (m, 2H), 2.37-2.22 (m, 2H), 2.20-1.98 (m, 2H), 1.79-1.63 (m, 2H).

Step 7. To a suspension of CH₃PPh₃Br (1.57 g, 4.39 mmol) in THF (50 mL) was added t-BuOK (0.49 g, 4.39 mmol). After stirring at 20° C. under N₂ for 1 hr, methyl 5-oxo-2,3,3a,4,6,6a-hexahydro-1H-pentalene-2-carboxylate (400 mg, 2.20 mmol) in THF (50 mL) was added, the reaction was stirred at 50° C. under N₂ for 2 h. The reaction was poured into H₂O (50 mL) and extracted with EtOAc (3×50 mL). The combined organic phase was washed with brine (150 mL), dried over Na₂SO₄, filtered and concentrated. The residue was purified by flash column (10% of EtOAc in PE) to give methyl 5-methylene-2,3,3a,4,6,6a-hexahydro-1H-pentalene-2-carboxylate (280 mg, 1 55 mmol) as colorless oil. ¹H NMR (400 MHz, CHLOROFORM-d) δ_{H}=4.86-4.70 (m, 2H), 3.65 (d, J=1.6 Hz, 3H), 2.94-2.59 (m, 2H), 2.56-2.38 (m, 3H), 2.21-1.91 (m, 4H), 1.72-1.60 (m, 1H), 1.52-1.39 (m, 1H).

Step 8. To a solution of methyl 5-methylene-2,3,3a,4,6,6a-hexahydro-1H-pentalene-2-carboxylate (280 mg, 1.55 mmol) in THF (5 mL) was added BH₃·Me₂S (43.0 mg, 3.11 mmol, 0.31 mL) slowly at 0° C. under N₂. After stirring at 20° C. under N₂ for 4 hrs. MeOH (3 mL) was added dropwise follow by NaOH (0.25 g, 6.21 mmol) (3 M, 2.1 mL) at 0° C. under N₂ and stirred for 0 5 hr. Then H₂02 (0.60 mL, 6.21 mmol) was added slowly at 0° C. under N₂ and the reaction was stirred at 20° C. under N₂ for 1 hr. The mixture cooled down to 0-5° C., Na₂S₂O₃ (20 mL, sat.) was added to the reaction mixture dropwise at 0-5° C. The mixture was stirred at this temperature for 1 hr and extracted with EtOAc (3×30 mL). The combined organic phase was washed Na₂S₂O₃ (100 mL, sat.), dried over Na₂SO₄, filtered and concentrated. The residue was purified by column (50% of EtOAc in PE) to give methyl 5-(hydroxymethyl)-1,2,3,3a,4,5,6,6a-octahydropentalene-2-carboxylate (200 mg, 1.01 mmol) as colorless oil. ¹H NMR (CHLOROFORM-d, 400 MHz) δ_{H}=3.68-3.65 (m, 3H), 3.61-3.55 (m, 2H), 2.86-2.73 (m, 1H), 2.64-2.55 (m, 1H), 2.53-2.44 (m, 1H), 2.25-2.10 (m, 2H), 2.07-2.00 (m, 2H), 1.88-1.77 (m, 1H), 1.75-1.66 (m, 1H), 1.62-1.52 (m, 2H), 1.13-0.99 (m, 1H), 0.89-0.73 (m, 1H).

Step 9. To a solution of methyl 5-(hydroxymethyl)-1,2,3,3a,4,5,6,6a-octahydropentalene-2-carboxylate (200 mg, 1.01 mmol) in DCM (10 mL) was added DMP (1284 mg, 3.03 mmol). After stirring at 20° C. for 2 hrs, the reaction was poured into NaHCO₃/Na₂S₂O₃ (30 mL, 1:1) and extracted with DCM (3×30 mL). The combined organic phase was washed with NaHCO₃/Na₂S₂O₃ (100 mL, 1:1), filtered and concentrated to give methyl 5-formyl-1,2,3,3a,4,5,6,6a-octahydropentalene-2-carboxylate (200 mg, 0.917 mmol) as yellow oil. ¹H NMR (CHLOROFORM-d, 400 MHz) δ_{H}=9.67-9.55 (m, 1H), 3.69-3.64 (m, 3H), 2.88-2.78 (m, 1H), 2.70-2.54 (m, 2H), 2.31-1.98 (m, 4H), 1.95-1.83 (m, 1H), 1.80-1.71 (m, 1H), 1.68-1.59 (m, 2H), 1.46-1.30 (m, 1H).

### Synthesis of Tert-butyl 3-(4-aminophenyl)-3-methylazetidine-1-carboxylate

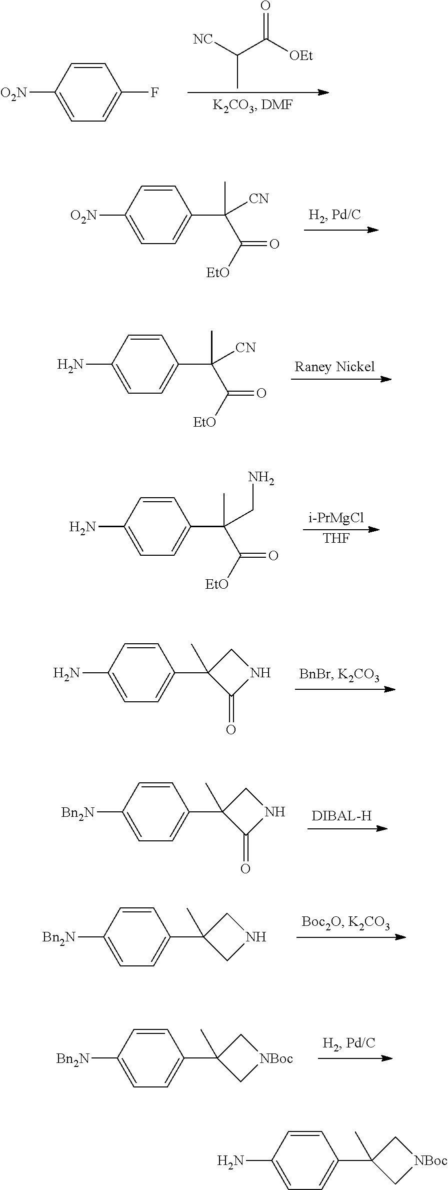

Step 1. To a solution of K₂CO₃ (21.6 g, 156 mmol) in DMF (100 mL) was added ethyl 2-cyanopropanoate (18.9 g, 149 mmol) at 150° C. The mixture was stirred at 150° C. for 20 mins. Then the reaction was cooled to 25° C. and stirred at 25° C. for 1.5 hr. A solution of 1-fluoro-4-nitrobenzene (20.0 g, 142 mmol) in DMF (20.0 mL) was added into the mixture at 0° C. and the resulting mixture was stirred at 25° C. for 12 hrs. The reaction mixture was added into ice water (100 mL). The reaction mixture was then adjusted to pH-5 by aqueous H₂SO₄/H₂O (20%, v/v) at 0° C. The mixture then chilled in a refrigerator for 2 hrs. The resulting mixture was extracted with EtOAc (3×200 mL). The combined organic phase was washed with brine (150 mL), dried over anhydrous Na₂SO₄, filtered and concentrated. The residue was purified by combi-flash column (EtOAc in PE=0%~7%) to give ethyl 2-cyano-2-(4-nitrophenyl)propanoate (12.3 g, 35% yield) as a yellow solid. ¹H NMR (400 MHz, DMSO-d₆) δ=8.38-8.28 (m, 2H), 7.86-7.78 (m, 2H), 4.28-4.20 (m, 2H), 2.01 (s, 3H), 1.21-1.16 (m, 3H).

Step 2. To a solution of ethyl 2-cyano-2-(4-nitrophenyl)propanoate (12.3 g, 49.5 mmol) in THF (100 mL) was added wet Pd/C (5.00 g, purity 10%) under Ar atmosphere. The suspension was degassed and purged with H₂ for 3 times. The reaction mixture was stirred under H₂ (15 Psi) at 25° C. for 48 hrs. The reaction mixture was filtered through celite. The filter cake was washed with MeOH/DCM (1/1, 600 mL). The resulting suspension was concentrated directly to give ethyl 2-(4-aminophenyl)-2-cyanopropanoate (10.0 g, 93% yield) as a yellow solid. ¹H NMR (400 MHz, DMSO-d₆) δ=7 12 (d, J=8.8 Hz, 2H), 6.60 (d, J=8.4 Hz, 214), 5.33 (s, 2H), 4.18 (q, J=7.2 Hz, 2H), 1.84 (s, 3H), 1.17 (t, J=7.2 Hz, 3H).

Step 3. To a solution of ethyl 2-(4-aminophenyl)-2-cyanopropanoate (10.0 g, 45.8 mmol) in ethanol (100 mL) was added Raney Nickel (5.00 g, 58.9 mmol) under Ar atmosphere. The suspension was degassed and purged with H₂ for 3 times. The resulting mixture was stirred under H₂ (30 Psi) at 30° C. for 28 hrs. The reaction mixture was filtered through celite. The filter cake was washed with MeOH/DCM (1/1, 500 mL). The resulting suspension was concentrated directly. The residue was purified by combi-flash column (EtOAc in PE=0%-50%). The desired fractions were concentrated to dryness in vacuo to give ethyl 3-amino-2-(4-aminophenyl)-2-methylpropanoate (7.00 g, 70% yield) as yellow oil. ¹H NMR (400 MHz, DMSO-d₆) δ=6.91 (d, J=8.4 Hz, 2H), 6.50 (d, J=8.4 Hz, 2H), 4.96 (s, 2H), 4.08-4.03 (m, 2H), 3.06 (d, J=12.8 Hz, 1H), 2.70 (d, J=12.8 Hz, 1H), 1.41 (s, 3H), 1.13 (t, J=7.2 Hz, 3H).

Step 4 To a solution of ethyl 3-amino-2-(4-aminophenyl)-2-methylpropanoate (4.00 g, 18.0 mmol) in THF (40.0 mL) was added i-PrMgCl (45 mL, 90.0 mmol) at 0° C. The mixture was stirred at 25° C. for 12 hrs. The reaction was quenched with aqueous NH₄Cl (60 mL) drop-wise. The resulting mixture was extracted with EtOAc (3×100 mL). The combined organic phase was washed with brine (60 mL), dried over anhydrous Na₂SO₄, filtered and concentrated The residue was purified by combi-flash column (EtOAc in PE=0%~60%) to give 3-(4-aminophenyl)-3-methylazetidin-2-one (3.50 g, 16.0 mmol) as a yellow solid. ¹H NMR (400 MHz, DMSO-d₆) δ=7.84 (s, 1H), 7.05-6.92 (m, 2H), 6.53 (d, J=8.4 Hz, 2H), 4.97 (s, 2H), 3.28-3.25 (m, 1H), 3.23-3.19 (m, 1H), 1.46 (s, 3H).

Step 5. To a solution of 3-(4-aminophenyl)-3-methylazetidin-2-one (3.20 g, 18.2 mmol) in DMF (30.0 mL) was added K₂CO₃ (7.53 g, 54.5 mmol) under N₂ atmosphere. Then BnBr (6.83 g, 40.0 mmol) was added into the reaction mixture. The reaction mixture was degassed and purged with N₂ for 3 times. The mixture was stirred at 20° C. for 6 hrs. The reaction was taken up in EtOAc (100 mL) and the organic layers was washed with water (50 mL) and brine (3×50 mL). The organic layers were then separated and dried over Na₂SO₄ before concentration to dryness. The crude was then purified by flash column chromatography eluting with 70% of EtOAc in PE to give 3-(4-(dibenzylamino)phenyl)-3-methylazetidin-2-one (5.61 g, 87% yield) as a white solid. ¹H NMR (400 MHz, DMSO-d₆) δ=7.90 (s, 1H), 7.41-7.26 (m, 10H), 7.13 (d, J=8.8 Hz, 2H), 6 70 (d, J=8.8 Hz, 2H), 4.73 (s, 4H), 3.33 (d, J=5.6 Hz, 1H), 3.25 (d, J=5.6 Hz, 1H), 1.50 (s, 3H).

Step 6. To a solution of 3-(4-(dibenzylamino)phenyl)-3-methylazetidin-2-one (5.00 g, 14.0 mmol) in toluene (50.0 mL) was added a solution of DIBAL-H (70.0 mL, 70.1 mmol) at 0° C. under N₂ atmosphere. The mixture was stirred at 110° C. for 14 hrs under N₂ atmosphere. The reaction was quenched with saturated aqueous potassium sodium tartrate (60 mL) drop-wise at 0° C. The resulting mixture was filtered. The filter cake was washed with EtOAc (150 mL). The organic layer was concentrated to give N,N-dibenzyl-4-(3-methylazetidin-3-yl)aniline (4.80 g, 80% yield) as yellow oil. ¹H NMR (400 MHz, DMSO-d₆) δ=7.32 (d, J=7.2 Hz, 4H), 7.28-7.22 (m, 6H), 6.94 (d, J=8.8 Hz, 2H), 6.62 (d, J=8.8 Hz, 2H), 4.67 (s, 4H), 3.66 (d, J=7.2 Hz, 2H), 3.28 (s, 2H), 1.47 (s, 3H).

Step 7. To a solution of N,N-dibenzyl-4-(3-methylazetidin-3-yl)aniline (3.50 g, 11.1 mmol) in THF (2.00 mL), water (2.00 mL) was added K₂CO₃ (303 mg, 2.19 mmol), tert-butoxycarbonyl tert-butyl carbonate (319 mg, 1.46 mmol) under N₂ atmosphere. The reaction was taken up in EtOAc (500 mL) and the organic layer was washed with 20 mL water then 3×20 mL saturated brine solution. The organics were then separated and dried over Na₂SO₄ before concentration to dryness. The crude was then purified by flash column chromatography eluting with 6% of EtOAc in PE. The desired fractions were concentrated to give tert-butyl 3-(4-(dibenzylamino)phenyl)-3-methylazetidine-1-carboxylate (180 mg, 56% yield) as a yellow solid. ¹H NMR (400 MHz, DMSO-d₆) δ=7.37-7.19 (m, 10H), 6.99 (d, J=8.8 Hz, 2H), 6.63 (d, J=8.8 Hz, 2H), 4.69 (s, 4H), 3.94 (d, J=7.6 Hz, 2H), 3.75 (d, J=6.4 Hz, 2H), 1.45 (s, 3H), 1.37 (s, 9H).

Step 8 To a solution of tert-butyl 3-(4-(dibenzylamino)phenyl)-3-methylazetidine-1-carboxylate (4.80 g, 10.8 mmol) in THF (20.0 mL), methanol (20.0 mL) was added wet Pd(OH)₂/C (500 mg, 20%) and wet Pd/C (500 mg, 10%) under N₂ atmosphere. The suspension was degassed and purged with H₂ for 3 times. The reaction mixture was stirred under H₂ (30 Psi) at 25° C. for 16 hr. After cooling to r.t., the mixture was filtered through Celite. The filtrate was concentrated. Tert-butyl 3-(4-aminophenyl)-3-methylazetidine-1-carboxylate (2.50 g, 88% yield) was obtained as colorless oil. ¹H NMR (400 MHz, DMSO-d₆) δ=6.97 (d, J=8.4 Hz, 2H), 6.59 (d, J=8.4 Hz, 2H), 5.01 (s, 2H), 4.01 (d, J=7.6 Hz, 2H), 3.82 (d, J=5.6 Hz, 2H), 1.52 (s, 3H), 1.44 (s, 9H).

### Compounds Prepared in Accordance with General Scheme 2

The compounds herein (such as a compound of Formula I or Formula II, or other compounds in Section I) may be prepared according to General Scheme 2, for example, from an intermediate as prepared in “Synthesis of additional intermediates for Table 2 compounds,” an intermediate that is commercially available, an intermediate as prepared elsewhere in this application or an intermediate that can be synthesized via a method known to those skilled in the art. Exemplary full syntheses are provided in Examples 48, 49, and 50 (Compound I-57 and Compound I-61). Compounds prepared in accordance with General Scheme 2 are provided in Table 2 below.

TABLE 2

| | Compound | | | |
|---|---|---|---|---|
| Example | No, | Structure | 1H NMR | LCMS |
| 134 | I-143 | 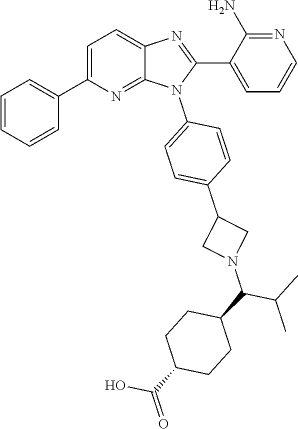 [SMILES: CC(C)C([C@H]1CC[C@H](C(=O)O)CC1)N1CC(c2ccc(-n3c(-c4cccnc4N)nc4ccc(-c5ccccc5)nc43)cc2)C1] | ¹HNMR (400 MHz, DMSO-d₆) δ = 12.25-11.67 (m, 1H), 8.27 (d, J = 8.4 Hz, 1H), 8.06-7.95 (m, 4H), 7.56-7.50 (m, 2H), 7.50-7.42 (m, 4H), 7.42-7.36 (m, 1H), 7.20 (dd, J = 1.6, 7.6 Hz, 1H), 6.95 (s, 2H), 6.42 (dd, J = 4.8, 7.6 Hz, 1H), 3.68 (br s, 3H), 3.16 (br s, 2H), 2.18- 2.05 (m, 1H), 2.03- 1.85 (m, 3H), 1.84- 1.72 (m, 1H), 1.65 (br d, J = 10.8 Hz, 2H), 1.48-1.35 (m, 1H), 1.33-1.15 (m, 4H), 0.90 (t, J = 6.4 Hz, 6H). | MS ESI calcd. for 600.3 [M + H]⁺ 601.3, found 601.1. |
| 135 | 1-144 | 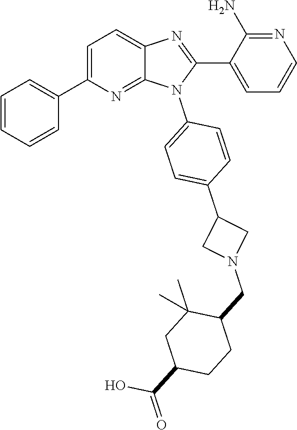 | ¹HNMR (400 MHz, DMSO-d₆) δ = 8.27 (d, J = 8.4 Hz, IH), 8.08-7.96 (m, 4H), 7.55-7.37 (m, 7H), 7.21 (d, J = 7.2 Hz, 1H), 6.96 (s, 2H), 6.43 (dd, J = 4.8, 7.6 Hz, 1H), 3.74- 3.61 (m, 3H), 3.12 (t, J = 6.4 Hz, 1H), 3.02 (t, J = 6.4 Hz, 1H), 2.46-2.34 (m, 3H), 1.74-1.44 (m, SH), 1.39-1.31 (m, 1H), 1.23-1.15 (m, 1H), 0.98 (s, 3H), 0.85 (s, 3H). | MS ESI calcd. for 586.31, [M + H]⁺ 587.31, found 587.3. |
| 136 | I-145 | 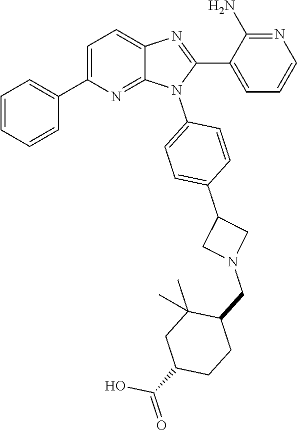 | . ¹HNMR (400 MHz, DMSO-d₆) δ = 8.27 (d, ] = 8.4 Hz, 1H), 8.09-7.90 (m, 4H), 7.64-7.35 (m, 7H), 7.21 (d, J = 7.2 Hz, 1H), 6.96 (s, 2H), 6.43 (dd, J = 5.2, 7.2 Hz, 1H), 3.73-3.61 (m, 3H), 3.12 (t, J = 6.4 Hz, 1H), 3.02 (t, J = 6.4 Hz, 1H), 2.47-2.34 (m, 3H), 1.74-1.43 (m, 5H), 1.40-1.30 (m, 1H), 1.19 (d, J = 4.4 Hz, 1H), 0,98 (s, 3H), 0.85 (s, 3H). | MS ESI calcd. for 586.31, [M + H]⁺ 587.31, found 587.3. |
| 137 | I-146 | 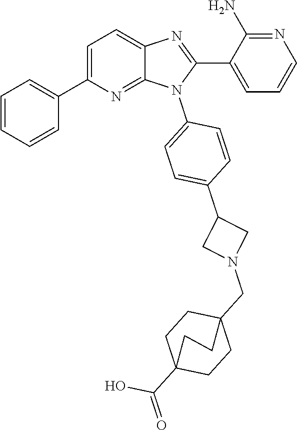 [SMILES: Nc1ncccc1-c1nc2ccc(-c3ccccc3)nc2n1-c1ccc(C2CN(CC34CCC(C(=O)O)(CC3)CC4)C2)cc1] | ¹HNMR (400 MHz, DMSO-d₆ δ = 8.26 (d, J = 8.4 Hz, 1H), 8.05-7.95 (m, 4H), 7.54-7.36 (m, 7H), 7.22-7.16 (m, 1H), 6.95 (s, 2H), 6.42 (dd, J = 7.6, 4.8 Hz, 1H), 3.68 (s, 3H), 3.15 (s, 2H), 2.18 (s, 2H), 1.68-1.57 (m, 6H), 1.41-1.30 (m, 6H). | MS ESI calcd. for 584.29, [M + H]⁺ 585.29, found 585.2. |
| 138 | I-147 | 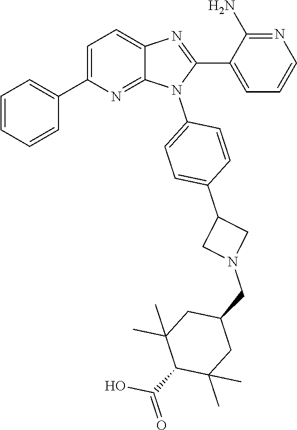 [SMILES: CC1(C)C[C@H](CN2CC(c3ccc(-n4c(-c5cccnc5N)nc5ccc(-c6ccccc6)nc54)cc3)C2)CC(C)(C)[C@H]1C(=O)O] | ¹HNMR (400 MHz, DMSO-d₆) δ = 8.36 (s, IH), 8.26 (d, J = 8.4 Hz, 1H), 8.06- 7.94 (m, 4H), 7.55- 7.36 (m, 7H), 7.20 (dd, J = 1.6, 7.6 Hz, 1H), 6.95 (s, 2H), 6.46-6.39 (m, 1H), 3.75-3.63 (m, 3H), 3.12-3.07 (m, 2H), 2.27 (d, J = 6.4 Hz, 2H), 1.90 (s, 1H), 1.76-1.63 (m, 1H), 1.54-1.44 (m, 2H), 1.08 (s, 6H), 0.89 (s, 6H), 0.77 (t, J = 12.8 Hz, 2H). | MS ESI calcd. for 614.34 [M + H]⁺ 615.34, found 615.2. |
| 139 | I-148 | 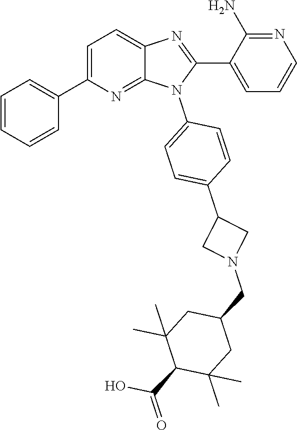 [SMILES: CC1(C)C[C@H](CN2CC(c3ccc(-n4c(-c5cccnc5N)nc5ccc(-c6ccccc6)nc54)cc3)C2)CC(C)(C)[C@@H]1C(=O)O] | ¹HNMR (400 MHz, DMSO-d₆) δ = 8.40- 8.33 (m, 1H), 8.26 (d, J = 8.4 Hz, 1H), 8.06-7.95 (m, 4H), 7.53-7.36 (m, 7H), 7.19 (dd, J = 2.0, 7.6 Hz, 1H), 6,94 (s, 2H), 6.47-6.38 (m, 1H), 3.71-3.63 (m, 3H), 3.09 (br t, J = 6.0 Hz, 2H), 2.30 (br d, J = 6.4 Hz, 2H), 1.96 (s, 1H), 1.76-1.64 (m, 1H), 1.46-1.34 (m, 2H), 1.26-1.19 (m, 2H), 1.08 (s, 6H), 0.90 (s, 6H) | MS ESI calcd. for 614.34 [M + H]⁺ 615.34, found 615.2. |
| 140 | I-149 | 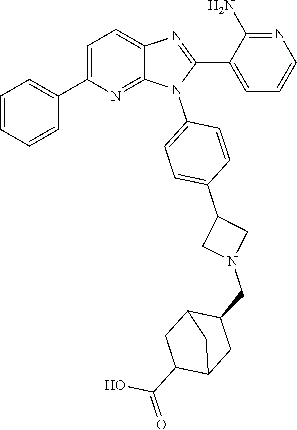 [SMILES: Nc1ncccc1-c1nc2ccc(-c3ccccc3)nc2n1-c1ccc(C2CN(C[C@H]3CC4CC3CC4C(=O)O)C2)cc1] | ¹HNMR (400 MHz, DMSO-d₆) δ = 8.30- 8.23 (m, 1H), 8.06- 7.95 (m, 4H), 7.55- 7.36 (m, 7H), 7.20 (dd, J = 1.6, 7.6 Hz, 1H), 6.95 (s, 2H), 6.42 (dd, J = 4.8, 7.6 Hz, 1H), 3.76- 3.61 (m, 3H), 3.13- 3.07 (m, 2H), 2.39- 2.27 (m, 2H), 2.25- 2.09 (m, 3H), 1.75- 1.64 (m, 1H), 1.50- 1.30 (m, 3H), 1.28- 1.15 (m, 2H), 1.11- 0.93 (m, 1H). | MS ESI calcd. For 570.21 [M + H]⁺ 571.21, found 571.1. |
| 141 | I-150 | 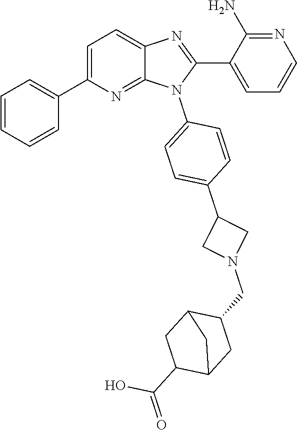 [SMILES: Nc1ncccc1-c1nc2ccc(-c3ccccc3)nc2n1-c1ccc(C2CN(C[C@@H]3CC4CC3CC4C(=O)O)C2)cc1] | . ¹HNMR (400 MHz, DMSO-d₆) δ = 8.26 (d, J = 8.4 Hz, 1H), 8.16 (s, 1H), 8.06-7.94 (m, 4H), 7.56-7.35 (m, 7H), 7.20 (dd, J = 1.6, 7.6 Hz, 1H), 6.94 (s, 2H), 6.42 (dd, J = 4.8, 7.6 Hz, 1H), 3.72 (s, 3H), 3.19 (br d, J = 2.0 Hz, 2H), 2.74-2.66 (m, 1H), 2.41 (br s, 2H), 2.20-2.14 (m, 1H), 1.94-1.77 (m, 2H), 1.66-1.54 (m, 1H), 1.51-1.39 (m, 3H), 1.29-1.17 (m, 1H), 0.78 (br dd, J = 4.4, 12.8 Hz, 1H) | MS ESI calcd. For 570.21 [M + H]⁺ 571.21, found 571.1. |
| 142 | I-151 | 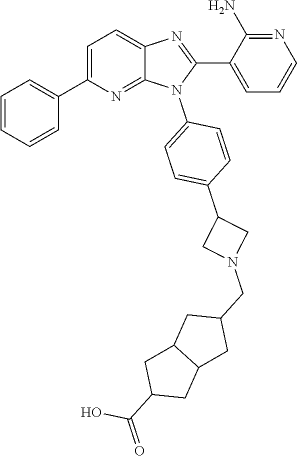 [SMILES: Nc1ncccc1-c1nc2ccc(-c3ccccc3)nc2n1-c1ccc(C2CN(CC3CC4CC(C(=O)O)CC4C3)C2)cc1] | ¹H NMR (METHANOL-d₄ 400 MHz) δ_{H}= 8.19 (d, J = 8.4 Hz, 1H), 8.08-7.86 (m, 4H), 7.67-7.48 (m, 4H), 7.46-7.30 (m, 4H), 6.52-6.44 (m, 1H), 4.58-4.39 (m, 2H), 4.36-4.17 (m, 3H), 3.30-3.16 (m, 2H), 2.89-2.69 (m, 1H), 2.68-2.49 (m, 3H), 2.32-2.03 (m, 3H), 1.88-1.53 (m, 3H), 1.44-1.30 (m, 1H), 1.22-0.86 (m, 1H). | MS ESI calcd. for 584.3 [M + H]⁺ 585.3, found 585.2. |
| 143 | I-152 | 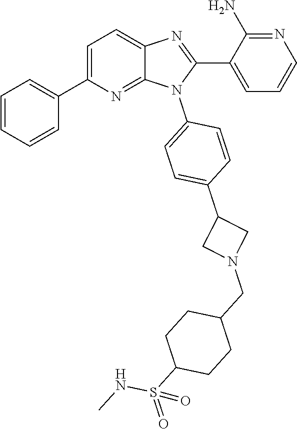 [SMILES: CNS(=O)(=O)C1CCC(CN2CC(c3ccc(-n4c(-c5cccnc5N)nc5ccc(-c6ccccc6)nc54)cc3)C2)CC1] | ¹HNMR (400 MHz, DMSO-d₆) δ = 8.26 (d, J = 8.4 Hz, 1H), 8.20 (s, 1H), 8.05- 7.94 (m, 2H), 7.56- 7.50 (m, 2H), 7.50- 7.43 (m, 4H), 7.42- 7.37 (m, 1H), 7.20 (dd, J = 2.0, 7.6 Hz, 1H), 6.93 (s, 2H). 6.89-6.80 (m, 1H), 6.42 (dd, J = 4.8, 7.6 Hz, 1H), 3.43 ~ 3.21 (m, 3H), 3.08- 2.87 (m, 1H), 2.69- 2.52 (m, 4H), 2.48- 2.43 (m, 1H), 2.17- 1.92 (m, 2H), 1.92- 1.57 (m, 3H), 1.56- 1.21 (m, 3H), 1.11- 0.72 (m, 2H). | MS ESI calcd. for 607.27 [M + H]⁺ 608.27, found 608.3. |
| 144 | I-153 | 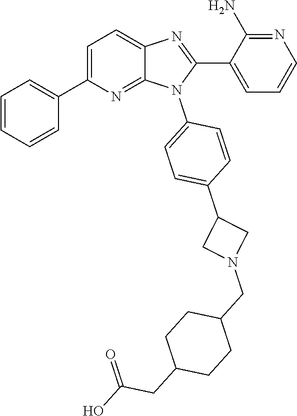 [SMILES: Nc1ncccc1-c1nc2ccc(-c3ccccc3)nc2n1-c1ccc(C2CN(CC3CCC(CC(=O)O)CC3)C2)cc1] | ¹HNMR (400 MHz, DMSO-d₆) δ = 8.26 (d, J = 8.4 Hz, 1H), 8.07-7.94 (m, 4H), 7.56-7.35 (m, 7H), 7.20 (dd, J = 1.6, 7.6 Hz, 1H), 6.96 (s, 2H), 6.42 (dd, J = 4.8, 7.6 Hz, 1H), 3.74-3.58 (m, 3H), 3.12-3.06 (m, 2H), 2.41-2.36 (m, 1H), 2.31-2.25 (m, 2H), 2.19-2.14 (m, 1H), 2.11-2.05 (m, 2H), 1.80-1.67 (m, 3H), 1.63-1.51 (m, 1H), 1.50-1.40 (m, 1H), 1.39-1.17 (m, 2H), 0.98-0.81 (m, 3H). | MS ESI calcd. for 572.29 [M + H]⁺ 573.29, found 573.3. |
| 145 | I-154 | 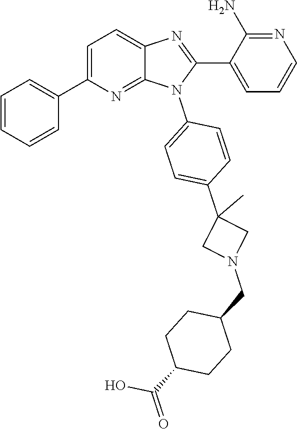 [SMILES: CC1(c2ccc(-n3c(-c4cccnc4N)nc4ccc(-c5ccccc5)nc43)cc2)CN(C[C@H]2CC[C@H](C(=O)O)CC2)C1] | ¹H NMR (400 MHz, DMSO-d₆) δ = 8.26 (d, J = 8.4 Hz, 1H), 8.07-7.93 (m, 4H), 7.52-7.36 (m, 7H), 7.17 (dd, J = 1.6, 7.6 Hz, 1H), 6.95 (s, 2H), 6.41 (dd, J = 4.8, 7.6 Hz, 1H), 3.26 (d, J = 6.4 Hz, 4H), 2.27 (d, J = 6.4 Hz, 2H), 2.14-2.03 (m, 1H), 1.87 (d, J = 11.2 Hz, 2H), 1.82-1.74 (m, 2H), 1.58 (s, 3H), 1.26 (dq, J = 2.8, 12.8 Hz, 3H), 0.95- 0.78 (m, 2H). | MS ESI calcd. for 572.29 [M + H]⁺ 573.29, found 573.3. |
| 146 | 1-155 | 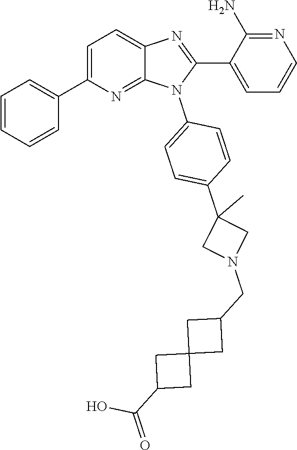 [SMILES: CC1(c2ccc(-n3c(-c4cccnc4N)nc4ccc(-c5ccccc5)nc43)cc2)CN(CC2CC3(C2)CC(C(=O)O)C3)C1] | ¹H NMR (400 MHz, DMSO-d₆) δ = 8.29-8.18 (m, 1H), 8.03-7.92 (m, 4H), 7.51-7.33 (m, 7H), 7.19 (d. J = 7.6 Hz, 1H), 6.47-6.38 (m, 1H), 3.37 (d, J = 6.4 Hz, 2H), 3.32-3.28 (m, 2H), 2.84-2.77 (m, IH), 2.44 (s, 2H), 2.17- 1.96 (m, 7H), 1.91 (d, J = 1.6 Hz, IH), 1.68-1.63 (m, 1H), 1.55 (s, 3H). | MS ESI calcd. for 584.29 [M + H]⁺ 585.29, found 585.3. |
| 147 | I-156 | 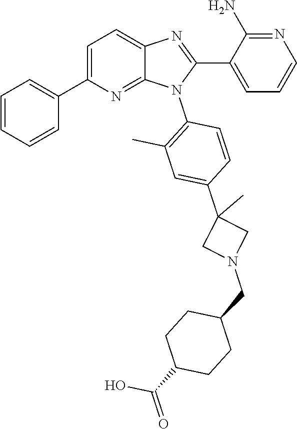 | .¹H NMR (400 MHz, MeOD) δ_{H} = 8.22 (d, J = 8.4 Hz, 1H), 8.01-7.89 (m, 4H), 7.45-7.33 (m, 5H), 7.32-7.27 (m, 1H), 7.23 (dd, J = 2.0, 8.0 Hz, 1H), 6.41 (dd, J = 5.2, 7.6 Hz, 1H), 4.41- 4.25 (m, 2H), 4.14 (d, J = 10.0 Hz, 2H), 2.95 (d, J = 7.2 Hz, 2H), 2.17-2.06 (m, 4H), 2.03-1.95 (m, 2H), 1.82 (d, J = 10.8 Hz, 2H), 1.74 (s, 3H), 1.69- 1.55 (m, 1H), 1.51- 1.37 (m, 2H), 1.11- 0.98 (m, 2H). | MS ESI calcd. for 586.31 [M + H]⁺ 587.31, found 587.3. |
| 148 | I-157 | 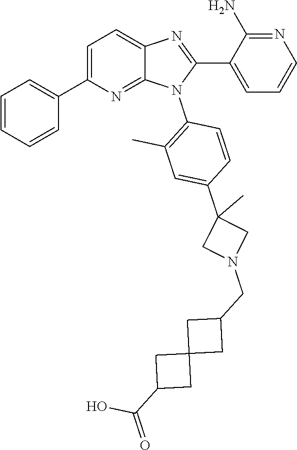 [SMILES: Cc1cc(C2(C)CN(CC3CC4(C3)CC(C(=O)O)C4)C2)ccc1-n1c(-c2cccnc2N)nc2ccc(-c3ccccc3)nc21] | . ¹H NMR (MeOD- d4, 400 MHz) δ_{H} = 8.22 (d, J = 8.4 Hz, 1H), 8.00-7.89 (m, 4H), 7.45-7.36 (m. 5H), 7.32-7.27 (m, 1H), 7.22 (dd, J = 2.0. 7.6 Hz, 1H), 6.41 (dd, J = 5.2, 7.6 Hz, 1H), 4.37 (dd, J = 4.8, 10.4 Hz, 2H), 4.15 (d, J = 10.4 Hz, 2H), 3.14 (d, J = 7.2 Hz, 2H), 2.46-2.37 (m, 1H), 2.46-2.37 (m, 1H), 2.33-2.24 (m, 3H), 2.21-2.11 (m, 3H), 2.08 (s, 3H), 1.86 (dd, J = 8.8, 10.8 Hz, 1H), 1.78 (dd, J = 8.8, 11.2 Hz, 1H), 1.73 (s, 3H). | ESI calcd. for 598.31 [M + H]⁺ 599.31, found 599.3 |

### Synthesis of Additional Intermediates for Table 3 Compounds

### Synthesis of trans-4-((4-aminophenethyl)(methyl)amino)cyclohexane-1-carboxylate

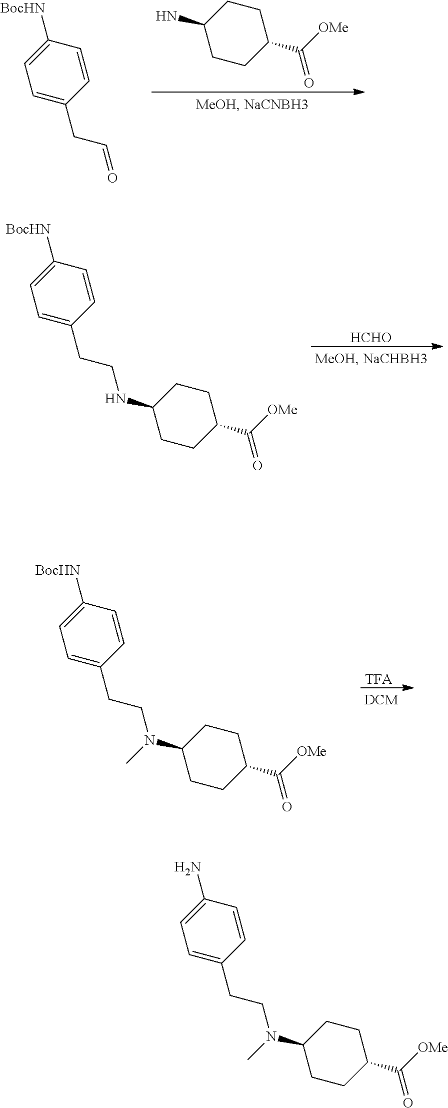

Step 1. To a solution of tert-butyl (4-(2-oxoethyl)phenyl)carbamate (550 mg, 2 34 mmol) and trans-methyl 4-aminocyclohexanecarboxylate (550 mg, 2.85 mmol) in methanol (5.00 mL) was added NaBH₃CN (550 mg, 8.75 mmol) at 0° C. The reaction mixture was stirred at 20° C. for 3 hrs under N₂ atmosphere. The reaction mixture was concentrated. Aq. NaHCO₃ (50 mL) was added. The resulting mixture was extracted with EtOAc (3×50 mL). The combined organic phase was washed with brine (50 mL), dried over anhydrous Na₂SO₄, filtered and concentrated to give methyl trans-4-((4-((tert-butoxycarbonyl)amino)phenethyl)amino)cyclohexane-1-(560 mg, crude) as a brown solid. ¹H NMR (400 MHz, CHLOROFORM-d) δ=7.24-7.20 (m, 2H), 7.10-7.05 (m, 2H), 6.44 (s, 1H), 3.59 (s, 3H), 3.56-3.49 (m, 1H), 2.99 (q, J=7.2 Hz, 1H), 2.93-2.88 (m, 2H), 2.82-2.76 (m, 2H), 2 62-2.54 (m, 1H), 2.26-2.11 (m, 2H), 2.03-1.98 (m, 2H), 1.97-1.89 (m, 4H), 1.44 (s, 9H).

Step 2. To a solution of methyl trans-4-((4-((tert-butoxycarbonyl)amino)phenethyl)amino)cyclohexane-1- (560 mg, 1.49 mmol) and HCHO (0.240 g, 2.97 mmol, 37% in water) in methanol (15 0 mL) was added NaBH₃CN (280 mg, 4.46 mmol). The mixture was stirred at 25° C. for 12 hrs. The reaction mixture was concentrated. Sat. aq. NaHCO₃ (50 mL) was added. The resulting mixture was extracted with EtOAc (3×50 mL). The combined organic phase was washed with brine (50 mL), dried over anhydrous Na₂SO₄, filtered and concentrated. The residue was purified by flash silica gel chromatography (eluent of 0%-4% MeOH in DCM) to give methyl trans-4-((4-((tert-butoxycarbonyl)amino)phenethyl)(methyl)amino)cyclohexane-1-carboxylate (330 mg, 57% yield) as a yellow solid. ¹H NMR (400 MHz, DMSO-d₆) δ 9.19 (s, 1H), 7.32 (d, J=8.4 Hz, 2H), 7.07 (d, J=8.4 Hz, 2H), 3.57 (s, 3H), 2.60-2.57 (m, 3H), 2.44-2.36 (m, 1H), 2.26-2.16 (m, 4H), 1.95-1.87 (m, 2H), 1 77-1.68 (m, 2H), 1.46 (s, 91), 1.38-1.18 (m, 5H).

Step 3. To a solution of methyl trans-4-((4-((tert-butoxycarbonyl)amino)phenethyl)(methyl)amino)cyclohexane-1-carboxylate (330 mg, 0.840 mmol) in DCM (3.00 mL) was added TFA (1.50 mL). The mixture was stirred at 25° C. for 12 hrs. The reaction mixture was concentrated to give methyl trans-4-((4-aminophenethyl)methyl)amino)cyclohexane-1-carboxylate (350 mg, crude) as a red solid ¹H NMR (400 MHz, DMSO-d₆) δ=9.55 (s, 1H), 7.33 (d, J=8.4 Hz, 2H), 7.17 (d, J=8.4 Hz, 2H), 3.59 (s, 3H), 3.35-3.28 (m, 2H), 3.25-3.17 (m, 1H), 2.80-2.74 (m, 3H), 2 37-2.29 (m, 1H), 2 04-1.96 (m, 4H), 1.79-1.73 (m, 2H), 1.62-1.49 (m, 2H), 1.48-1.39 (m, 2H).

### Synthesis of methyl 1-(4-((3-amino-6-phenylpyridin-2-yl)amino)phenethyl)pyrrolidine-3-carboxylate

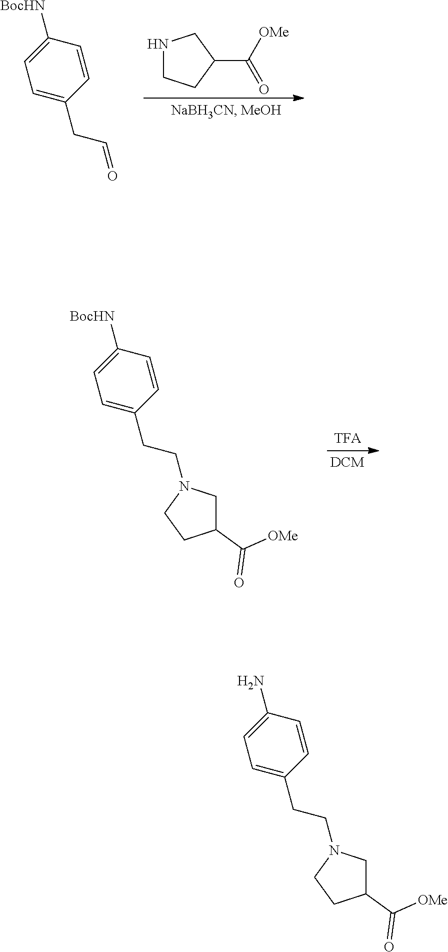 [SMILES: CC(C)(C)OC(=O)Nc1ccc(CC=O)cc1 ; COC(=O)C1CCNC1 ; COC(=O)C1CCN(CCc2ccc(NC(=O)OC(C)(C)C)cc2)C1 ; COC(=O)C1CCN(CCc2ccc(N)cc2)C1]

Step 1. To a solution of methyl 1-(4-aminophenethyl)pyrrolidine-3-carboxylate (450 mg, 1.81 mmol) and 2-chloro-3-nitro-6-phenyl-pyridine (425 mg, 1.81 mmol) in 1,4-dioxane (20 mL) was added Cs₂CO₃ (1.77 g, 5.44 mmol) and Pd(dppf)Cl₂ (132 mg, 0.18 mmol). The reaction mixture was stirred at 80° C. for 5 hrs, then filtered and concentrated. The residue was purified by flash chromatography (eluent 0-2% MeOH in DCM) to give methyl 1-(4-((3-nitro-6-phenylpyridin-2-yl)amino)phenethyl)pyrrolidine-3-carboxylate (300 mg, 37% yield). LCMS Rₜ=0.508 min in 1 min chromatography, purity 82.0%, MS ESI calcd. for 446.20 [M+H]⁺ 447.20, found 447.1.

Step 2. To a solution of methyl 1-(4-((3-nitro-6-phenylpyridin-2-yl)amino)phenethyl)pyrrolidine-3-carboxylate (300 mg, 0.672 mmol) in THF (10 mL) was added Pd/C (200 mg, wet, 10% purity) under Ar atmosphere. The suspension was degassed and purged with H₂ for 3 times The reaction mixture was stirred under H₂ (15 Psi) at 25° C. for 3 hrs. The mixture was filtered through celite and washed with solvents (MeOH: DCM=1:5, 4×10 mL). The filtrate was concentrated to give methyl 1-(4-((3-amino-6-phenylpyridin-2-yl)amino)phenethyl)pyrrolidine-3-carboxylate (198 mg, 71% yield) as a yellow solid. ¹H NMR (400 MHz, DMSO-d₆) δ=7.94-7.89 (m, 2H), 7.79-7.74 (m, 1H), 7.70 (d, J=8.4 Hz, 1H), 7.43-7.34 (m, 3H), 7.27-7.21 (m, 2H), 7.21-7.13 (m, 2H), 6.98-6.94 (m, 1H), 5.29-5.21 (m, 2H), 3.67-3.62 (m, 3H), 3.24-3.09 (m, 4H), 2.99-2.91 (m, 1H), 2.78-2.68 (m, 2H), 2.25-1.90 (m, 4H).

### Synthesis of ethyl 4-((4-amino-3-fluorobenzo(methyl)amino)-2,6-dimethylcyclohexane-1-carboxylate

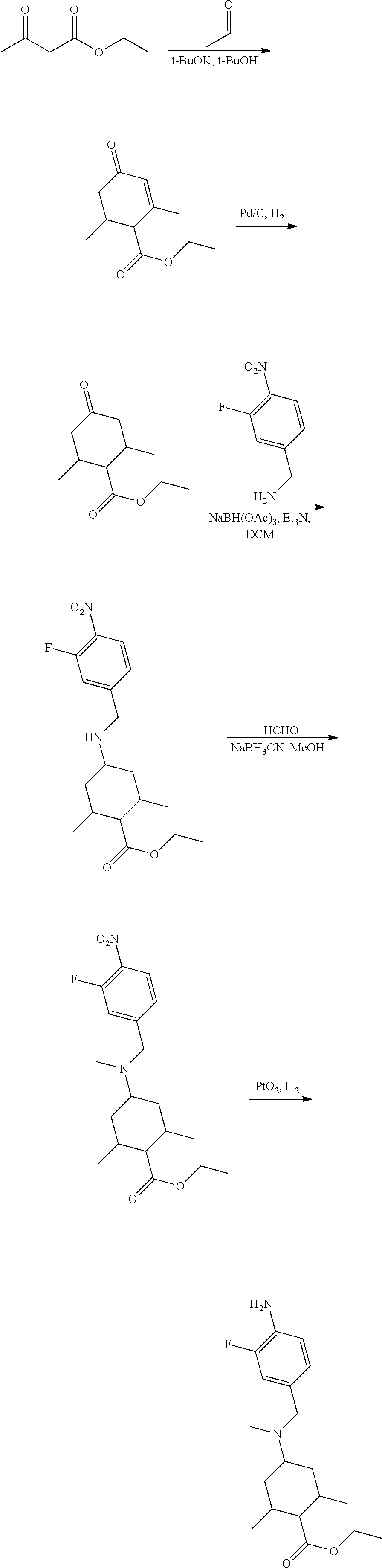

Step 1. To a solution of ethyl 3-oxobutanoate (6.20 g, 47.7 mmol) in tert-butanol (10 mL) was added acetaldehyde (1.00 g, 22.7 mmol) and t-BuOK (0.51 g, 4.54 mmol) at 25° C. The mixture was stirred at 80° C. for 12 hrs. The reaction mixture was concentrated directly. The residue was purified by flash silica gel chromatography (eluent of 0-15% EtOAc in PE). Ethyl 2,6-dimethyl-4-oxo-cyclohex-2-ene-1-carboxylate (2.6 g, 13.2 mmol, 58% yield) was obtained as a colorless oil. ¹H NMR (400 MHz, CHLOROFORM-d) δ=5.89 (s, 1H), 4.19-4.11 (m, 2H), 2.96 (d, J 6.8 Hz, 1H), 2.60-2.43 (m, 2H), 2.05 (dd, J=2.4, 10.4, 17.6 Hz, 1H), 1.90 (d, J=1.2 Hz, 3H), 1.25-1.20 (m, 3H), 1 04-0.98 (m, 3H).

Step 2. To a solution of ethyl 2,6-dimethyl-4-oxocyclohex-2-ene-1-carboxylate (6.00 g, 30.6 mmol) in THF (50 mL) was added Pd/C (3.00 g, 10% purity) under Ar atmosphere. The suspension was degassed and purged with H₂ for 3 times. The reaction mixture was stirred under H₂ (30 Psi) at 25° C. for 12 hrs. The reaction mixture was filtered via a celite pad, and the filter cake was washed with DCM:MeOH=1:1 (800 mL). The filtered suspension was concentrated directly. The residue was purified by combi-flash column (EtOAC in PE=0-8%) to afford ethyl 2,6-dimethyl-4-oxocyclohexane-1-carboxylate (3.20 g, 16.1 mmol, 53% yield) as a yellow oil. ¹H NMR (400 MHz, CHLOROFORM-d) δ=4.21-4.15 (m, 2H), 2.76-2.63 (m, 3H), 2.26 (s, 1H), 2.19 (d, J=4.0 Hz, 1H), 2.17-2.13 (m, 1H), 2.13-2.07 (m, 1H), 1.30-1.26 (m, 3H), 1.02 (d, J=6.8 Hz, 6H).

Step 3. To a solution of (3-fluoro-4-nitrophenyl)methanamine (2.00 g, 9.68 mmol) in DCM (25 ml) was added ethyl 2,6-dimethyl-4-oxocyclohexane-1-carboxylate (2.50 g, 12.6 mmol) and TEA (1.96 g, 19.4 mmol) at 20° C. The resulting mixture was stirred at 20° C. for 30 mins. NaBH(OAc)₃ (6.16 g, 29.0 mmol) was added into the reaction and stirred at 45° C. for 12 hrs under N₂ atmosphere. The reaction was concentrated directly. The residue was purified by combi-flash column (EtOAC in PE=0~15%) to afford ethyl 4-((3-fluoro-4-nitrobenzyl)amino)-2,6-dimethylcyclohexane-1-carboxylate (1.78 g, 5 05 mmol, 52% yield) as a yellow oil ¹H NMR (CDCl₃, 400 MHz) δ=8.02 (t, J=8.0 Hz, 1H), 7.36 (d, J=12.0 Hz, 1H), 7.30-7.26 (m, 1H), 4.18 (q, J=7.2 Hz, 2H), 3.85 (s, 2H), 2.92 (t, J=2.8 Hz, 1H), 2.11-1.94 (m, 2H), 1.76 (d, J=13.2 Hz, 2H), 1.61 (t, J=10.8 Hz, 1H), 1.28 (t, J=7.2 Hz, 3H), 1.16 (dt, J=3.2, 13.2 Hz, 2H), 0.86 (d, J=6.8 Hz, 6H).

Step 4. To a solution of ethyl 4-((3-fluoro-4-nitrobenzyl)amino)-2,6-dimethylcyclohexane-1-carboxylate (1.80 g, 5.11 mmol) in methanol (20 mL) and DCM (5 mL) was added formaldehyde (2.07 g, 25.5 mmol) at 20° C. The reaction was stirred at 20° C. for 30 mins. NaBH₃CN (963 mg, 15.3 mmol) was added into the resulting mixture and stirred at 20° C. for 4 hrs under N₂ atmosphere: water (15 mL) was added to the reaction mixture. The resulting mixture was extracted with DCM (3×20 mL). The combined organic phase was washed with brine (10 mL), dried over anhydrous Na₂SO₄, filtered and concentrated The residue was purified by combi-flash column (EtOAc in PE=0%-11%) to afford ethyl 4-((3-fluoro-4-nitrobenzyl)(methyl)amino)-2,6-dimethylcyclohexane-1-carboxylate (1.60 g, 4.37 mmol, 85% yield) as yellow oil. ¹H NMR (400 MHz, CHLOROFORM-d) δ=8.02 (t, J=8.0 Hz, 1H), 7.34 (d, J=12.0 Hz, 1H), 7.29 (d, J=8.8 Hz, 1H), 4.20 (q, J=7.2 Hz, 2H), 3.59 (s, 2H), 2 50 (t, J=2.8 Hz, 1H), 2.23-2.12 (m, 2H), 2.10 (s, 3H), 2.03-1.94 (m, 2H), 1.71-1.63 (m, 1H), 1.30 (t, J=7.2 Hz, 3H), 1.21-1.10 (m, 2H), 0.85 (d, J=6.8 Hz, 6H).

Step 5. To a solution of ethyl 4-((3-fluoro-4-nitrobenzyl)(methyl)amino)-2,6-dimethylcyclohexane-1-carboxylate (1.60 g, 4.37 mmol) in ethyl acetate (20 mL) was added PtO₂ (800 mg) under Ar atmosphere. The suspension was degassed and purged with H₂ for 3 times. The reaction mixture was stirred under H₂ (15 Psi) at 25° C. for 12 hrs. The reaction mixture was filtered via a celite pad, and the filter cake was washed with DCM:MeOH=1:1 (800 mL). The filtered suspension was concentrated directly. The residue was purified by combi-flash column (EtOAC in PE=0%~25%) to afford ethyl 4-((4-amino-3-fluorobenzyl)methyl)amino)-2,6-dimethylcyclohexane-1-carboxylate (920 mg, 2.73 mmol, 63% yield) as yellow oil. ¹H NMR (CDCl₃, 400 MHz) δ=7.00-6.93 (m, 1H), 6.89 (d, J=8.0 Hz, 1H), 6.72 (t, J=8.8 Hz, 1H), 4.19 (q, J=7.2 Hz, 2H), 3.64 (s, 2H), 3.40 (s, 2H), 2.38 (t, J=2.8 Hz, 1H), 2.25-2.18 (m, 2H), 2.08-1.97 (m, 5H), 1.64-1.61 (m, 1H), 1.30 (t, J=7.2 Hz, 3H), 1 14-1.04 (m, 2H), 0.85 (d, J=6.8 Hz, 6H).

### Synthesis of methyl 4-((4-amino-3-fluorobenzyl) (methyl) amino) bicycle 12.2.21 octane-1-carboxylate

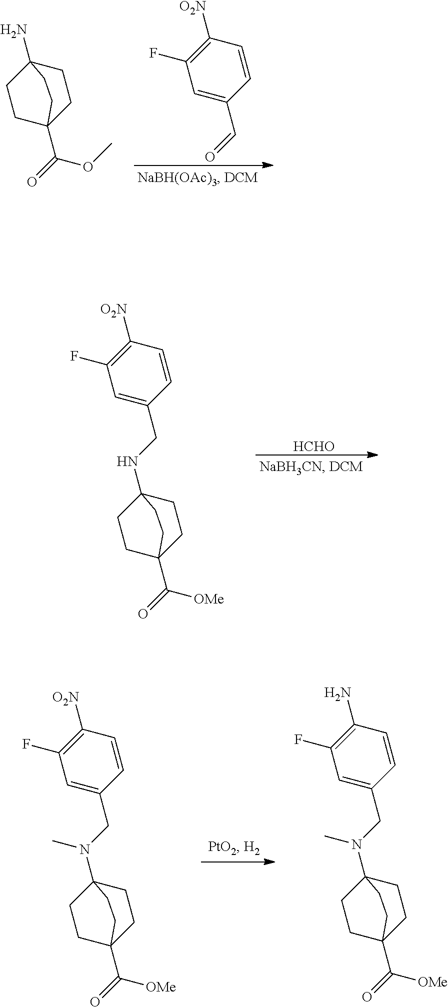 [SMILES: COC(=O)C12CCC(N)(CC1)CC2 ; O=Cc1ccc([N+](=O)[O-])c(F)c1 ; COC(=O)C12CCC(NCc3ccc([N+](=O)[O-])c(F)c3)(CC1)CC2 ; "HCHO" ; COC(=O)C12CCC(N(C)Cc3ccc([N+](=O)[O-])c(F)c3)(CC1)CC2 ; COC(=O)C12CCC(N(C)Cc3ccc(N)c(F)c3)(CC1)CC2]

Step 1. To a solution of 3-fluoro-4-nitrobenzaldehyde (800 mg, 4.73 mmol) and methyl 4-aminobicyclo[2.2.2]octane-1-carboxylate (954 mg, 5.20 mmol) in DCM (10 mL) was added TEA (479 mg, 4.73 mmol) and NaBH(OAc)₃ (1504 mg, 7.10 mmol). The mixture was stirred at 25° C. for 12 hrs Water (10 mL) was added to the residue. The resulting mixture was extracted with DCM (3×10 mL). The combined organic phase was washed with brine (10 mL), dried over anhydrous Na₂SO₄, filtered and concentrated. The residue was purified by flash silica gel chromatography (eluent of 0~70% EtOAc in PE) to give methyl 4-((3-fluoro-4-nitrobenzyl)amino)bicyclo[2.2.2]octane-1-carboxylate (1270 mg, 3.78 mmol) as light yellow solid. ¹H NMR (DMSO-d₆, 400 MHz) δ=8.08 (t, J=8.0 Hz, 1H), 7.55-7.37 (m, 2H), 3.74 (s, 2H), 3.55 (s, 3H), 1.79-1.72 (m, 6H), 1.55-1.48 (m, 6H).

Step 2. To a solution of methyl 4-((3-fluoro-4-nitrobenzyl)amino)bicyclo[2.2.2]octane-1-carboxylate (1270 mg, 3.78 mmol) in Methanol (20 mL) was added formaldehyde (613 mg, 7.55 mmol). The mixture was stirred at 25° C. for 0.5 hr. Then NaBH₃CN (475 mg, 7.55 mmol) was added into the mixture. The mixture was stirred at 25° C. for 12 hrs. The reaction mixture was concentrated. Water (10 mL) was added to the residue. The resulting mixture was extracted with DCM (3×10 mL). The combined organic phase was washed with brine (10 mL), dried over anhydrous Na₂SO₄, filtered and concentrated. The residue was purified by flash silica gel chromatography (eluent of 0-30% EtOAc in PE) to give methyl 4-((3-fluoro-4-nitrobenzyl)(methyl)amino)bicyclo[2.2.2]octane-1-carboxylate (900 mg, 2.56 mmol) as a light yellow solid. ¹H NMR (DMSO-d₆, 400 MHz) δ=8.09 (t, J=8.0 Hz, 1H), 7.49-7.33 (m, 2H), 3.61 (s, 2H), 3.56 (s, 3H), 2.02 (s, 3H), 1.81-1.74 (m, 6H), 1.65-1.58 (m, 6H).

Step 3. To a solution of methyl 4-((3-fluoro-4-nitrobenzyl)(methyl)amino)bicyclo[2.2.2]octane-1-carboxylate (900 mg, 2.57 mmol) in ethanol (8 mL) and THF (8 mL) was added PtO₂ (300 mg) under N₂ atmosphere. The suspension was degassed and purged with H₂ for 3 times. The reaction mixture was stirred under H₂ (30 Psi) at 25° C. for 12 hrs. The reaction mixture was filtered and the filtrate was concentrated to dryness. The residue was used in the next step directly. Compound methyl 4-((4-amino-3-fluorobenzyl) (methyl) amino) bicycle[2.2.2]octane-1-carboxylate (630 mg, crude) as light yellow oil. LCMS Rt=0.347 min in 1 min chromatography, purity 92% MS ESI calcd. for 320.19, [M+H]⁺ 321.19, found 321.1.

### Synthesis of cis-methyl 4-[(4-amino-3-fluoro-phenyl)methyl-ethyl-amino]cyclohexanecarboxylate

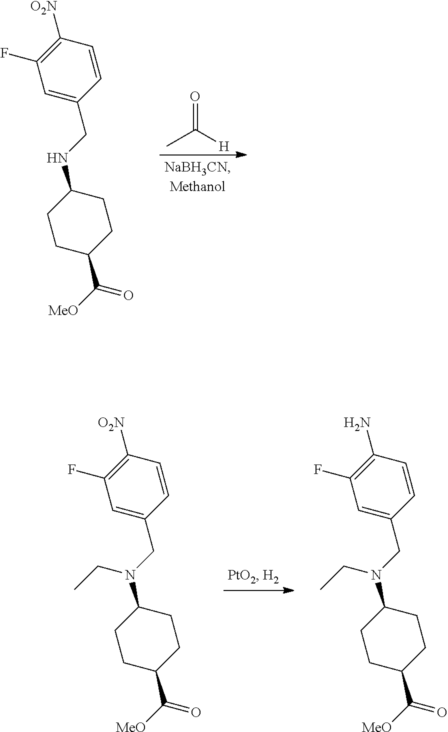

Step 1. To a solution of cis-methyl 4-[(3-fluoro-4-nitro-phenyl)methylamino]cyclohexanecarboxylate (250 mg, 0.806 mmol) in methanol (1 mL) was added acetaldehyde (177 mg, 1.61 mmol) NaBH₃CN (76.0 mg, 1.21 mmol). The mixture was stirred at 25° C. for 2 hrs. The reaction mixture was filtered. The filtrate was concentrated. The residue was dissolved in water (20 mL). The resulting suspension was extracted with EtOAc (3×20 mL). The combined organic phase was washed with brine (20 mL), water (20 mL), dried over anhydrous Na₂SO₄, filtered and concentrated. The residue was purified by combi-flash column (EtOAc in PE=20%) to give cis-methyl 4-[ethyl-[(3-fluoro-4-nitro-phenyl)methyl]amino]cyclohexanecarboxylate (200 mg, 73% yield) as a white solid. LCMS Rₜ=0.437 min in 1 min chromatography, MS ESI calcd. for 338.16 [M+H]⁺ 339.16 found 339.1.

Step 2. To a solution cis-methyl 4-[ethyl-[(3-fluoro-4-nitro-phenyl)methyl]amino]cyclohexanecarboxylate (230 mg, 0.680 mmol) in ethyl acetate (5 mL) was added PtO₂ (230 mg) under N₂ atmosphere. The suspension was degassed and purged with H₂ for 3 times. The reaction mixture was stirred under H₂ (15 Psi) at 25° C. for 12 hrs. The reaction mixture was filtered via a celite pad, and the filter cake was washed with DCM/MeOH=10/1 (50 mL). The filtrate was concentrated to give cis-methyl 4-[(4-amino-3-fluoro-phenyl)methyl-ethyl-amino]cyclohexanecarboxylate (150 mg, 72% yield) as a yellow solid. LCMS Rₜ=0.400 min in 1 min chromatography, MS ESI calcd. for 308.19 [M+H]⁺ 309.19 found 309.1.

### Synthesis of methyl trans-4-((4-amino-3-fluorobenzyl)(ethyl)amino)cyclohexane-1-carboxylate

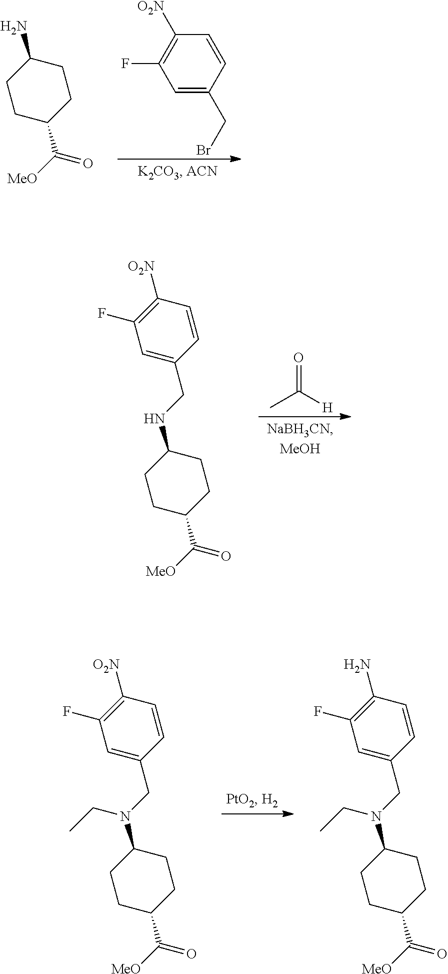

Step 1. To a solution of methyl (1r,4r)-4-aminocyclohexane-1-carboxylate (1.00 g, 5.16 mmol) and 4-(bromomethyl)-2-fluoro-1-nitrobenzene (1.09 g, 4.65 mmol) in MeCN (15 mL) was added K₂CO₃ (2.14 g, 15.5 mmol). The reaction mixture was stirred at 25° C. for 12 hrs. The mixture was diluted with H₂O (20 mL), extracted with EtOAc (3×20 mL), washed with brine (20 mL). The organic layer was dried over Na₂SO₄ and concentrated. The residue was purified by combi-flash column (EtOAc in PE=0%-48%) to afford methyl trans-4-((3-fluoro-4-nitrobenzyl)amino)cyclohexane-1-carboxylate (1.00 g, 62% yield) as a yellow solid. ¹H NMR (DMSO-d₆, 400 MHz) δ=8.09 (t, J=8.4 Hz, 1H), 7.53 (d, J=12.8 Hz, 1H), 7.40 (d, J=8 4 Hz, 1H), 3.83 (s, 2H), 3.56 (s, 3H), 2.35-2.23 (m, 2H), 1.97-1.81 (m, 4H), 1.35-1.22 (m, 2H), 1.11-0.99 (m, 2H).

Step 2. To a solution of methyl trans-4-((3-fluoro-4-nitrobenzyl)amino)cyclohexane-1-carboxylate (800 mg, 2.58 mmol) in methanol (10 mL) was added acetaldehyde (2.84 g, 25.8 mmol) at 25° C. The resulting mixture was stirred at 20° C. for 30 mins under N₂ atmosphere. NaBH₃CN (972 mg, 15.5 mmol) was added into the reaction and stirred at 30° C. for 18 hrs. The reaction was concentrated directly. The residue was purified by combi-flash column (EtOAc in PE=0%-18%) to afford methyl trans-4-(ethyl(3-fluoro-4-nitrobenzyl)amino)cyclohexane-1-carboxylate (1.00 g, crude) as yellow oil. ¹H NMR (CDCl₃, 400 MHz) δ_{H}=7.99 (t, J=8.0 Hz, 1H), 7.37 (d, J=12.0 Hz, 1H), 7.27-7.23 (m, 1H), 3.74-3.55 (m, 5H), 2.62-2.46 (m, 3H), 2.20 (tt, J=3.6, 12.0 Hz. 1H), 2.07-2.02 (m, 2H), 1.89 (d, J=10.8 Hz, 2H), 1.50-1.36 (m, 2H), 1.34-1.25 (m, 2H), 0.98 (t, J=7.2 Hz, 3H).

Step 3. To a solution of methyl (1r,4r)-4-(ethyl(3-fluoro-4-nitrobenzyl)amino)cyclohexane-1-carboxylate (1.00 g, 2.96 mmol) in ethyl acetate (15 mL) was added PtO₂ (300 mg) under Ar atmosphere. The suspension was degassed and purged with H₂ for 3 times. The reaction mixture was stirred under H₂ (15 Psi) at 25° C. for 12 hrs. The reaction mixture was filtered via a celite pad, and the filter cake was washed with DCM/MeOH=10/1 (500 mL). The filtrate was concentrated. The residue was purified by combi-flash column (EtOAc in PE=0%~20%) to afford methyl trans-4-((4-amino-3-fluorobenzyl)(ethyl)amino)cyclohexane-1-carboxylate (350 mg, 38% yield) as yellow oil. ¹H NMR (CDCl₃, 400 MHz) δ_{H}=7.01 (d, J=12.0 Hz, 1H), 6.87 (d, J=8.0 Hz, 1H), 6.69 (t, J=8.8 Hz, 1H), 3.70-3.54 (m, 5H), 2.57-2.44 (m, 3H), 2.19 (tt, J=3.6, 12.0 Hz, 1H), 2.02 (d, J=12.4 Hz, 2H), 1.87 (d, J=10.8 Hz, 2H), 1.49-1.35 (m, 2H), 1.34-1.23 (m, 2H), 0.97 (t, J=7.2 Hz, 3H).

### Synthesis of trans-4-((4-amino-3-fluorobenzyl)(methyl)amino)cyclohexane-1-carboxylate

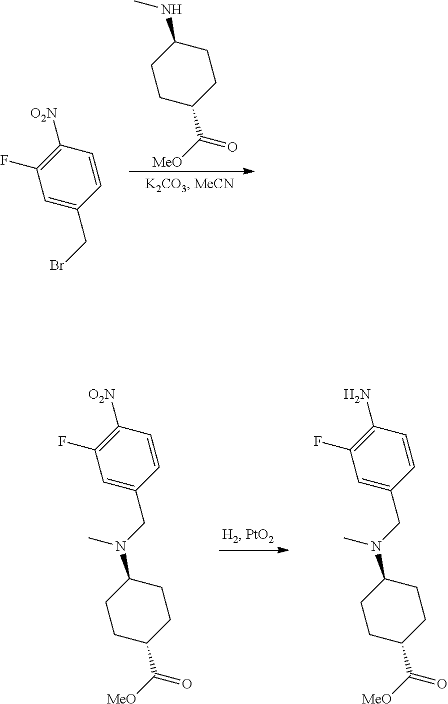 [SMILES: O=[N+]([O-])c1ccc(CBr)cc1F ; CN[C@H]1CC[C@H](C(=O)OC)CC1 ; COC(=O)[C@H]1CC[C@H](N(C)Cc2ccc([N+](=O)[O-])c(F)c2)CC1 ; COC(=O)[C@H]1CC[C@H](N(C)Cc2ccc(N)c(F)c2)CC1]

Step 1. To a solution of methyl trans-4-(methylamino)cyclohexane-1-carboxylate (2.92 g, 17.1 mmol) and 4-(bromomethyl)-2-fluoro-1-nitrobenzene (5.00 g, 21.4 mmol) in MeCN (40 mL) was added K₂CO₃ (7.07 g, 51.2 mmol). The reaction mixture was stirred at 25° C. for 2 hrs. The mixture was diluted with H₂O (20 mL), extracted with EtOAc (3×20 mL), washed with brine (20 mL). The organic layer was dried over Na₂SO₄ and concentrated to give product. The residue was purified by flash silica gel chromatography (eluent of 12% EtOAc in PE). Methyl trans-4-((3-fluoro-4-nitrobenzyl)(methyl)amino)cyclohexane-1-carboxylate (3.60 g, 66% yield) as a yellow liquid. LCMS Rₜ=0.496 & 0.920 min in 2.5 min chromatography, purity 99.2%, MS ESI calcd. For 324 15 [M+H]⁺ 325 15, found 325.3.

Step 2. To a solution of methyl trans-4-((3-fluoro-4-nitrobenzyl)(methyl)amino)cyclohexane-1-carboxylate (3.60 g, 11.1 mmol) in ethanol (30.0 mL) was added PtO₂ (1.80 g). The reaction mixture was stirred at 25° C. for 2 hrs under H₂ (15 psi). The mixture was filtered via a celite pad and the filter cake was washed with DCM:MeOH=1:1 (500 mL). The filtrate was concentrated to give crude methyl trans-4-((4-amino-3-fluorobenzyl)(methyl)amino)cyclohexane-1-carboxylate (3.70 g, crude) as a yellow solid.

### Synthesis of cis-4-((4-amino-3-fluorobenzyl)(methyl)amino)cyclohexane-1-carboxylate

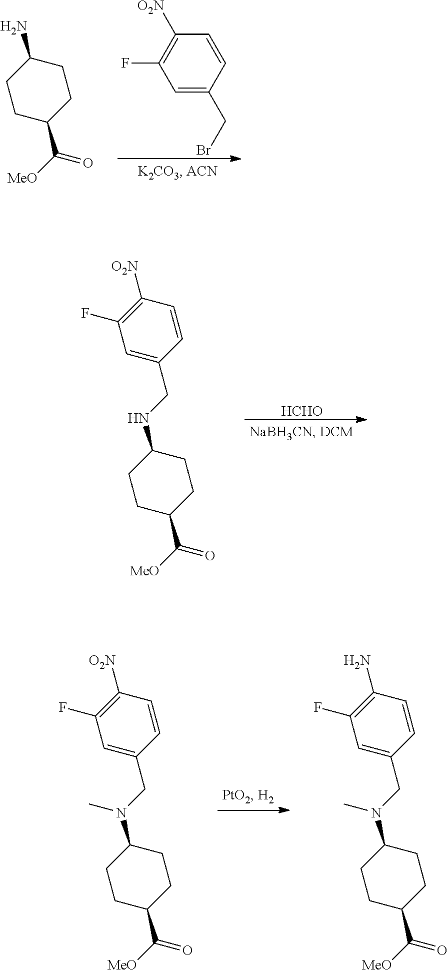 [SMILES: COC(=O)[C@H]1CC[C@@H](N)CC1 ; O=[N+]([O-])c1ccc(CBr)cc1F ; COC(=O)[C@H]1CC[C@@H](NCc2ccc([N+](=O)[O-])c(F)c2)CC1 ; "HCHO" ; COC(=O)[C@H]1CC[C@@H](N(C)Cc2ccc([N+](=O)[O-])c(F)c2)CC1 ; COC(=O)[C@H]1CC[C@@H](N(C)Cc2ccc(N)c(F)c2)CC1]

Step 1. To a solution of methyl cis-4-aminocyclohexane-1-carboxylate (5.00 g, 25.9 mmol) and 4-(bromomethyl)-2-fluoro-1-nitrobenzene (6.67 g, 28.5 mmol) in MeCN (50 mL) was added K₂CO₃ (10.7 g, 77.7 mmol). The reaction mixture was stirred at 25° C. for 2 hrs. The mixture was diluted with H₂O (100 mL), extracted with EtOAc (3×30 mL), washed with brine (100 mL). The organic layer was dried over Na₂SO₄ and concentrated. The residue was purified by combi-flash column (EtOAc in PE=24%) to give methyl cis-4-((3-fluoro-4-nitrobenzyl)amino)cyclohexane-1-carboxylate (8.00 g, 99% yield) as a yellow liquid. ¹H NMR (400 MHz, DMSO-d₆) δ=8.13-8.07 (m, 1H), 7.57-7.51 (m, 1H), 7.43-7.39 (m, 1H), 3.79 (s, 2H), 3.60 (s, 3H), 2.43-2.25 (m, 2H), 1.92-1.81 (m, 2H), 1.56-1.40 (m, 6H).

Step 2. To a solution of methyl cis-4-((3-fluoro-4-nitrobenzyl)amino)cyclohexane-1-carboxylate (2.00 g, 6.44 mmol) in methanol (20 mL) was added formaldehyde (387 mg, 12.9 mmol) and NaBH₃CN (607 mg, 9.67 mmol). The mixture was stirred at 25° C. for 2 hrs. The reaction mixture was filtered. The filtrate was concentrated The residue was dissolved in water (50 mL). The resulting suspension was extracted with EtOAc (3×30 mL). The combined organic phase was washed with brine (50 mL), water (50 mL), dried over anhydrous Na₂SO₄, filtered and concentrated. The residue was purified by combi-flash column (EtOAc in PE=14%) to give methyl cis-4-((3-fluoro-4-nitrobenzyl)(methyl)amino)cyclohexane-1-carboxylate (1.30 g, 62% yield) as a yellow solid. ¹H NMR (400 MHz, DMSO-d₆) δ=8.17-8.02 (m, 1H), 7.51-7.31 (m, 2H), 3.61 (s, 5H), 2.58 (s, 1H), 2.41 (s, 1H), 2.08 (s, 3H), 2.05-1.99 (m, 2H), 1.67-1.57 (m, 2H), 1.54-1.42 (m, 4H).

Step 3. To a solution methyl cis-4-((3-fluoro-4-nitrobenzyl)(methyl)amino)cyclohexane-1-carboxylate (300 mg, 0.925 mmol) in ethyl acetate (5 mL) was added wet PtO₂ (10%, 60.0 mg) under N₂ atmosphere. The suspension was degassed and purged with H₂ for 3 times. The reaction mixture was stirred under H₂ (15 Psi) at 25° C. for 12 hrs. The reaction mixture was filtered via a celite pad, and the filter cake was washed with DCM/MeOH=10/1 (50 mL). The filtrate was concentrated to give methyl cis-4-((4-amino-3-fluorobenzyl)methyl)amino)cyclohexane-1-carboxylate (280 mg, crude) as a yellow solid. ¹H NMR (400 MHz, DMSO-d₆) δ=10.12 (s, 1H), 8.63 (d, J=8.4 Hz, 1H), 8.07-7.91 (m, 3H), 7.61 (d, J=8.8 Hz, 1H), 7.54-7.48 (m, 3H), 7.27 (d, J=12.0 Hz, 1H), 7.21 (d, J=8.4 Hz, 1H), 3.62 (s, 3H), 3.57 (s, 2H), 2.54 (s, 2H), 2.11 (s, 31H), 1.72-1.33 (m, 8H).

### Synthesis of methyl trans-4-((4-aminobenzyl)(isobutyl)amino) cyclohexane-1-carboxylate

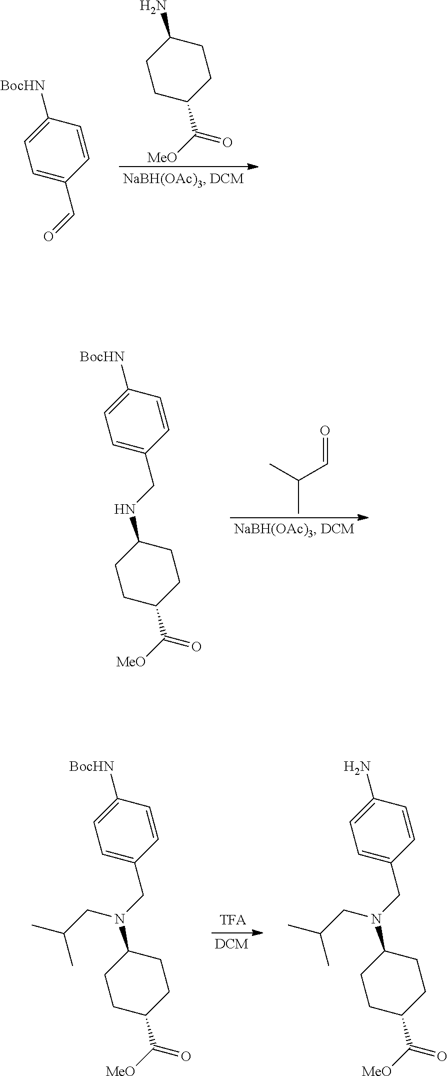

Step 1. To a solution of methyl trans-4-aminocyclohexane-1-carboxylate (3.06 g, 15.8 mmol) in DCM (100 mL) was added TEA (1.60 g, 15.8 mmol), tert-butyl (4-formylphenyl)carbamate (3.50 g, 15.8 mmol) and NaBH(OAc)₃ (10.0 g, 47.5 mmol) at 25° C. The mixture was stirred at 25° C. for 12 hrs. Water (100 mL) was added to the reaction mixture. The reaction mixture was then adjusted to pH-7 by aq. NaHCO₃ (1 N). The resulting mixture was extracted with DCM (3×50 mL). The combined organic phase was washed with brine (50 mL), water (50 mL), dried over anhydrous Na₂SO₄, filtered and concentrated. The residue was purified by flash chromatography (eluent of 0-10% MeOH in DCM) to give methyl trans-4-((4-((tert-butoxycarbonyl) amino) benzyl) amino) cyclohexane-1-carboxylate (6.00 g, 84% yield) as a white solid. ¹HNMR (400 MHz, DMSO-d₆) δ=9 30 (s, 1H), 7.39 (d, J=8.4 Hz, 2H), 7.24 (d, J=8.4 Hz, 2H), 4.27-4.18 (m, 1H), 3.73 (s, 2H), 3.57 (s, 3H), 2.49-2.42 (m, 1H), 2.24 (tt, J=3.6, 12.0 Hz, 1H), 2.01-1.94 (m, 2H), 1.93-1.90 (m, 1H), 1.88-1.87 (m, 1H), 1.46 (s, 9H), 1.30 (m, 2H), 1.20-1.09 (m, 2H).

Step 2. To a solution of isobutyraldehyde (133 mg, 1 38 mmol) in DCM (10.0 mL) was added methyl trans-4-((4-((tert-butoxycarbonyl) amino) benzyl) amino) cyclohexane-1-carboxylate (500 mg, 1.38 mmol) and NaBH(OAc)₃ (877 mg, 4.14 mmol) at 25° C. The mixture was stirred at 25° C. for 2 hrs. Water (10 mL) was added and the reaction mixture was then adjusted to pH-7 by aq. NaHCO₃ (1 M). The resulting mixture was extracted with DCM (3×10 mL). The combined organic phase was washed with brine (10 mL), dried over anhydrous Na₂SO₄, filtered and concentrated to give methyl trans-4-((4-((tert-butoxycarbonyl)amino)benzyl) (isobutyl)amino) cyclohexane-1-carboxylate (520 nig, 90% yield) as light yellow oil, which was used for the next step without further purification. ¹HNMR (400 MHz, DMSO-d₆) δ=9.22 (s, 1H), 7.35 (d, J=8.0 Hz, 2H), 7.15 (d, J=8.4 Hz, 2H), 3.55 (s, 3H), 3.46 (s, 2H), 2.35 (t, J=7.6 Hz, 1H), 2.25-2.12 (m, 3H), 1.91 (d, J=11.2 Hz, 2H), 1.74 (d, J=11.2 Hz, 2H), 1.61-1.53 (m, 1H), 1.46 (s, 9H), 1.32-1.19 (m, 4H), 0.77 (d, J=6.4 Hz, 6H).

Step 3. To a solution of methyl trans-4-((4-((tert-butoxycarbonyl)amino)benzyl)(isobutyl)amino) cyclohexane-1-carboxylate (520 mg, 1.24 mmol) in DCM (3.00 mL) was added TFA (142 mg, 1.24 mmol) at 25° C. The mixture was stirred at 25° C. for 12 hrs. The reaction mixture was concentrated directly to give methyl trans-4-((4-aminobenzyl)(isobutyl)amino) cyclohexane-1-carboxylate (380 mg, 96% yield) as light yellow oil, which was used for the next step without further purification. LCMS Rₜ=0.331 min in 1 min chromatography, purity 95%, MS ESI calcd. for 318.23 [M+H]⁺ 319.23, found 319.2.

### Synthesis of methyl trans-4-((4-aminobenzyl)(ethyl)amino)cyclohexane-1-carbaoxylate

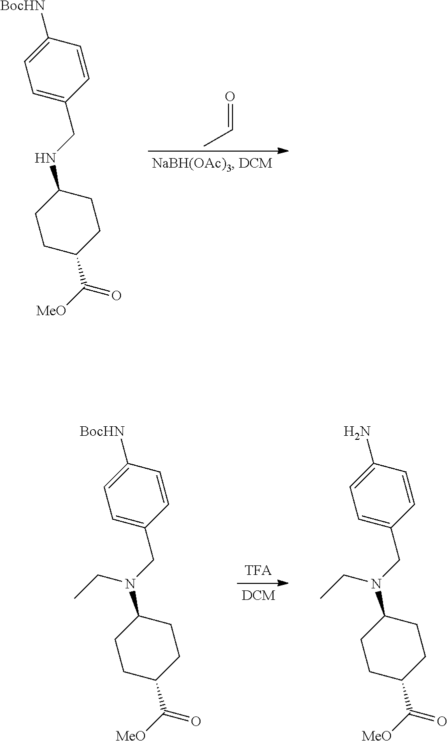

Step 1. To a solution of acetaldehyde (152 mg, 1.38 mmol) in DCM (10 mL) was added methyl trans-4-((4-((tert-butoxycarbonyl)amino)benzyl)amino)cyclohexane-1-carboxylate (500 mg, 1.38 mmol) and NaBH(OAc)₃ (877 mg, 4.14 mmol) at 25° C. The mixture was stirred at 25° C. for 2 hrs. Water (10 mL) was added and the reaction mixture was then adjusted to pH-7 by aq NaHCO₃(I M). The resulting mixture was extracted with DCM (3×10 mL). The combined organic phase was washed with brine (10 mL), water (10 mL), dried over anhydrous Na₂SO₄, filtered and concentrated. The reaction mixture was concentrated directly to give methyl trans-4-((4-((tert-butoxycarbonyl)amino)benzyl)(ethyl) amino)cyclohexane-1-carboxylate (470 mg, 87% yield) as light yellow oil, which was used for the next step without further purification. ¹HNMR (400 MHz, DMSO-d₆) δ=9.22 (s, 1H), 7.35 (d, J=8.4 Hz, 2H), 7.16 (d, J=8.4 Hz, 2H), 3.56 (s, 3H), 3.47 (s, 2H), 3.17 (s, 1H), 2.44-2.43 (m, 2H), 2.25-2 17 (m, 1H), 1.93-1.91 (m, 2H), 1.79-1.74 (m, 2H), 1.46 (s, 9H), 1.34-1.23 (m, 4H), 0.90 (t, J=7.2 Hz, 3H).

Step 2. To a solution of methyl trans-4-((4-((tert-butoxycarbonyl)amino) benzyl)(ethyl)amino)cyclohexane-1-carboxylate (480 mg, 1.23 mmol) in DCM (3.00 mL) was added TFA (140 mg, 1.23 mmol) at 25° C. The mixture was stirred at 25° C. for 1 hr. Water (10 mL) was added to the reaction mixture. The reaction mixture was concentrated directly to give methyl trans-4-((4-aminobenzyl)(ethyl)amino)cyclohexane-1-carboxylate (340 mg, 95% yield) as light yellow oil, which was used for the next step without further purification. LCMS Rₜ=0.302 min in 1 min chromatography, purity 96%, MS ESI calcd. for 290.20 [M+H]⁺ 291.20, found 291.1.

### Synthesis of methyl trans-4-(isopropyl(4-nitrobenzyl)amino)cyclohexane-1-carboxylate

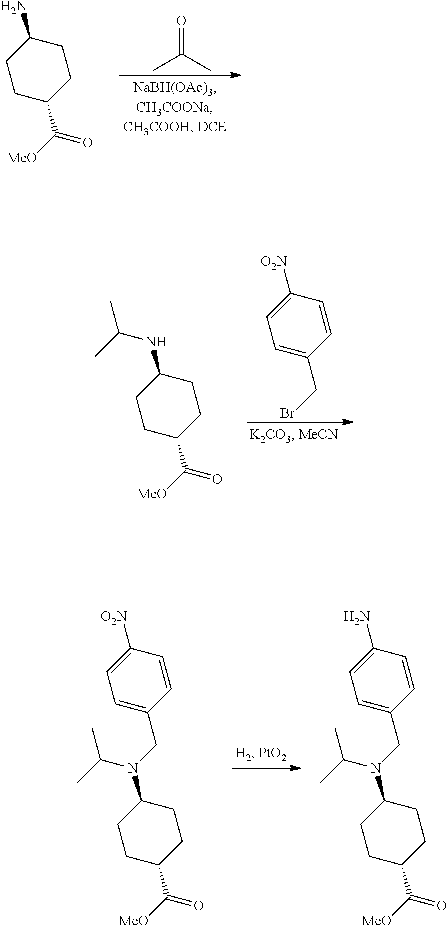

Step 1. To a solution of acetone (1.21 g, 20.8 mmol) in DCE (52.0 mL) was added CH₃COONa (6 60 g, 80.5 mmol), methyl trans-4-aminocyclohexane-1-carboxylate hydrochloride (5.20 g, 26.8 mmol). After stirring at 25° C. for 2 hrs, NaBH(OAc)₃ (17.1 g, 77.1 mmol) was added at 40° C. The mixture was stirred at 40° C. for 2 hrs. The reaction mixture was quenched with aq. NaHCO₃ (50 mL) drop-wise. The resulting mixture was extracted with DCM (3×50 mL). The combined organic phase was washed with brine (100 mL), water (100 mL), dried over anhydrous Na₂SO₄, filtered and concentrated to give methyl trans-4-(isopropylamino)cyclohexane-1-carboxylate (3.30 g, 62% yield) as a light yellow solid, which was used for the next step without further purification. ¹HNMR (400 MHz, DMSO-d₆) δ=3.58 (s, 3H), 3.07-3.01 (m, 1H), 2.70-2.59 (m, 1H), 2.23 (tt, J=3.2, 12.0 Hz, 1H), 1.93-1.88 (m, 4H), 1.84 (s, 1H), 1.42-1.30 (m, 2H), 1.18-1.08 (m, 2H), 1.03 (d, J=6.4 Hz, 6H).

Step 2. To a solution of methyl trans-4-(isopropylamino)cyclohexane-1-carboxylate (1.50 g, 7.53 mmol) in MeCN (15.0 mL) was added 1-(bromomethyl)-4-nitro-benzene (1.79 g, 8.28 mmol) and K₂CO₃ (2.08 g, 15.1 mmol) at 25° C. The mixture was stirred at 25 for 24 hrs. The reaction mixture was concentrated directly. The residue was purified by flash silica gel chromatography (eluent of 0~5% EtOAc in PE) to give methyl trans-4-(isopropyl(4-nitrobenzyl)amino)cyclohexane-1-carboxylate (1.00 g, 40% yield) as light yellow oil. ¹HNMR (400 MHz, DMSO-d₆) δ=8.24-8 22 (m, 2H), 7.66-7.63 (m, 2H), 3.78 (s, 2H), 3 55 (s, 3H), 2.97 (q, J=6.4 Hz, 1H), 2.52-2.51 (m, 1H), 2.23-2.14 (m, 1H), 1.90-1.72 (m, 4H), 1.30 (t, J=10.4 Hz, 4H), 0.98 (d, J=6.4 Hz, 6H).

Step 3. To a solution of methyl trans-4-(isopropyl(4-nitrobenzyl)amino)cyclohexane-1-carboxylate (900 mg, 2.69 mmol) in ethyl acetate (5.00 mL) was added PtO₂ (400 mg, 0.176 mmol) under N₂. The reaction mixture was degassed and purged with H₂ for 3 times. The mixture was stirred at 25° C. for 12 hrs under H₂ (15 psi). The reaction mixture was concentrated directly to give methyl trans-4-((4-aminobenzyl)(isopropyl)amino)cyclohexane-1-carboxylate (900 mg, 2 96 mmol) as light yellow oil, which was used for the next step without further purification. LCMS Rₜ=0.346 min in 1 min chromatography, purity 63%, MS ESI calcd. for 304.43 [M+H]⁺ 305.43, found 305.2.

### Synthesis of methyl 2-(trans-4-((4-aminobenzyl)(methyl)amino)cyclohexyl)acetate

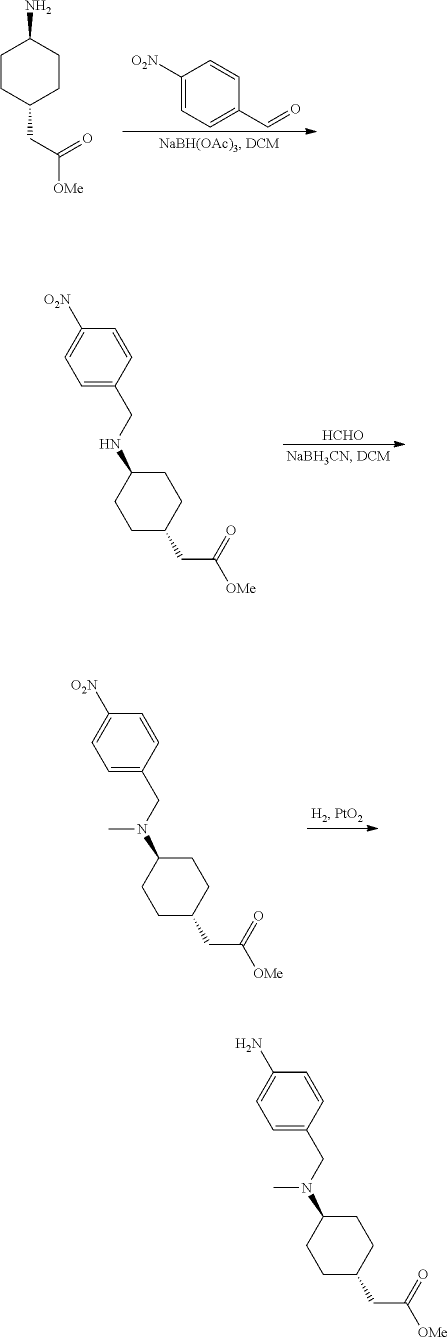 [SMILES: COC(=O)C[C@H]1CC[C@H](N)CC1 ; O=Cc1ccc([N+](=O)[O-])cc1 ; COC(=O)C[C@H]1CC[C@H](NCc2ccc([N+](=O)[O-])cc2)CC1 ; "HCHO" ; COC(=O)C[C@H]1CC[C@H](N(C)Cc2ccc([N+](=O)[O-])cc2)CC1 ; COC(=O)C[C@H]1CC[C@H](N(C)Cc2ccc(N)cc2)CC1]

Step 1. To a solution of methyl 2-(trans-4-aminocyclohexyl)acetate (1.00 g, 5.84 mmol) in DCM (15 mL) was added 4-nitrobenzaldehyde (883 mg, 5.84 mmol), TEA (1.6 mL, 11.7 mmol) under N₂ atmosphere. Then, NaBH(OAc)₃ (3.71 g, 17.5 mmol) was added into the reaction mixture. The reaction mixture was degassed and purged with N₂ for 3 times. The mixture was stirred at 20° C. for 12 hrs. Aq.NaHCO₃ (15 mL) was added to the residue. The resulting mixture was extracted with DCM (20 mL*3). The combined organic phase was washed with brine (5 mL), dried over anhydrous Na₂SO₄, filtered and concentrated. The crude product was purified by column chromatography (SiO₂, MeOH/DCM=3%). Methyl 2-(trans-4-((4-nitrobenzyl)amino)cyclohexyl)acetate (1 50 g, 4.85 mmol, 83% yield) was obtained as yellow oil. ¹HNMR (400 MHz, CHLOROFORM-d) δ=8 18 (d, J=8.8 Hz, 2H), 7.51 (d, J=8.8 Hz, 2H), 3.95 (s, 2H), 3.66 (s, 3H), 2.52-2.41 (m, 1H), 2.19 (d, J=6.4 Hz, 2H), 2.00-1.92 (m, 2H), 1.84-1.70 (m, 3H), 1.28-1.15 (m, 2H), 1.08-0.92 (m, 2H).

Step 2. To a solution of methyl methyl 2-(trans-4-((4-nitrobenzyl)amino)cyclohexyl)acetate (1.50 g, 4.90 mmol) and HCHO (1.5 mL, 14.7 mmol, 37% in water) in methanol (15 mL) was added NaBH₃CN (920 mg, 14.7 mmol). The resulting mixture was stirred at 25° C. for 2 hrs. The reaction mixture was concentrated. Aq NaHCO₃ (5 mL) was added. The resulting mixture was extracted with EtOAc (3×10 mL). The combined organic phase was washed with brine (5 mL), dried over anhydrous Na₂SO₄, filtered and concentrated. The crude product was purified by column chromatography (SiO₂, MeOH/DCM=2%) to give methyl 2-(trans-4-(methyl(4-nitrobenzyl)amino)cyclohexyl)acetate (780 mg, 2.37 mmol, 48% yield) as yellow oil. ¹HNMR (400 MHz, CHLOROFORM-d) δ=8.16 (d, J=8.4 Hz, 2H), 7.50 (d, J=8.4 Hz, 2H), 3.67 (s, 3H), 3.65 (s, 2H), 2.48-2.37 (m, 1H), 2.21 (s, 1H), 2.19 (s, 4H), 1.87 (t, J=12.4 Hz, 4H), 1.80-1.67 (m, 1H), 1.44-1.31 (m, 2H), 1.09-0.94 (m, 2H).

Step 3. To a solution of methyl 2-(trans-4-(methyl(4-nitrobenzyl)amino)cyclohexyl)acetate (200 mg, 0.624 mmol) in ethyl acetate (3 mL) was added PtO₂ (100 mg) under N₂ atmosphere. The suspension was degassed and purged with H₂ for 3 times. The reaction mixture was stirred under H₂ (15 Psi) at 25° C. for 2 hr. The reaction mixture was filtered and the filtrate was concentrated directly to give methyl 2-(trans-4-((4-aminobenzyl)(methyl)amino)cyclohexyl)acetate (170 mg, 0.585 mmol, 94% yield) as yellow oil. ¹HNMR (400 MHz, CHLOROFORM-d) δ=7.08 (d, J=8.4 Hz, 2H), 6.67-6.60 (m, 2H), 3.66 (s, 3H), 3.59 (s, 2H), 2.48-2.35 (m, 1H), 2.19 (s, 1H), 2.18-2.15 (m, 4H), 1.91-1.79 (m, 4H), 1 77-1.66 (m, 1H), 1.42-1.30 (m, 2H), 1.07-0.92 (m, 2H).

### Synthesis of Methyl 3-((4-aminobenzyl)(methyl)amino)cyclopentane-1-carboxylate

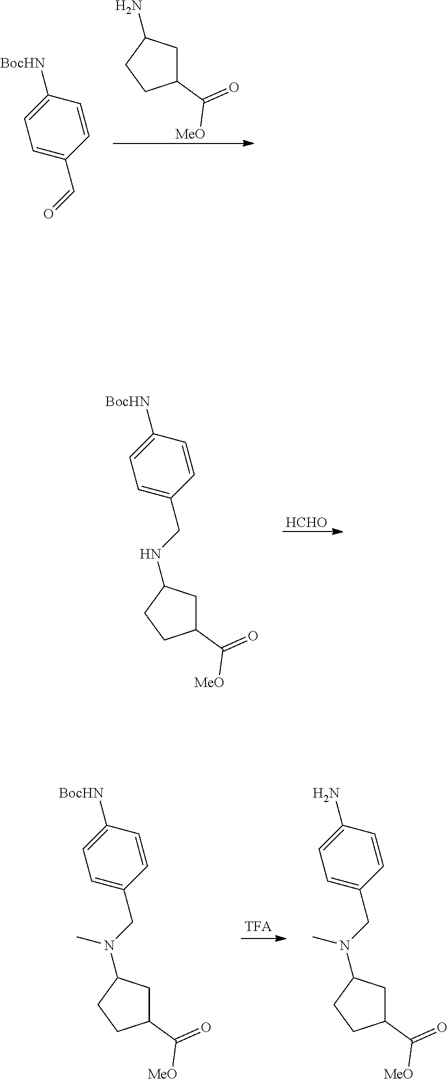 [SMILES: CC(C)(C)OC(=O)Nc1ccc(C=O)cc1 ; COC(=O)C1CCC(N)C1 ; COC(=O)C1CCC(NCc2ccc(NC(=O)OC(C)(C)C)cc2)C1 ; "HCHO" ; COC(=O)C1CCC(N(C)Cc2ccc(NC(=O)OC(C)(C)C)cc2)C1 ; COC(=O)C1CCC(N(C)Cc2ccc(N)cc2)C1]

Step 1. To a solution of tert-butyl N-(4-formylphenyl)carbamate (3.71 g, 16.8 mmol) in DCM (20 mL) was added methyl 3-aminocyclopentanecarboxylate (3.00 g, 21.0 mmol) and NaBH(OAc)₃ (4.44 g, 21.0 mmol) at 25° C. The mixture was stirred at 25° C. for 16 hr. The reaction mixture was concentrated directly. The residue was purified by flash silica gel chromatography (eluent of 0-10% MeOH in DCM). Compound methyl 3-((4-((tert-butoxycarbonyl)amino)benzyl)amino)cyclopentane-1-carboxylate (1.80 g, 5.17 mmol, 25% yield) was obtained as yellow oil LCMS Rt=0.692 min in 1.5 min chromatography, purity 83.2%, MS ESI calcd. for 348.20, [M+H]⁺ 349.20, found 349.2.

Step 2. To a solution of methyl 3-((4-((tert-butoxycarbonyl)amino)benzyl)amino)cyclopentane-1-carboxylate (1.80 g, 5.17 mmol) in Methanol (10 mL) was added formaldehyde (1.26 g, 15.5 mmol) and NaBH₃CN (3.28 g, 15.5 mmol) at 25° C. The mixture was stirred at 25° C. for 2 hr. The reaction mixture was concentrated Aq. NaHCO₃ (20 mL) was added to the residue. The resulting mixture was extracted with DCM (3×10 mL). The combined organic phase was washed with brine (5 mL), water (5 mL), dried over anhydrous Na₂SO₄, filtered and concentrated. The residue was purified by flash silica gel chromatography (eluent of 0~5% MeOH in DCM). Compound methyl 3-((4-((tert-butoxycarbonyl)amino)benzyl)(methyl)amino)cyclopentane-1-carboxylate (1.50 g, 4.14 mmol, 80% yield) was obtained as yellow oil. LCMS Rt=0.465 min in 1 min chromatography, purity 98%, MS ESI calcd. for 362.22, [M+H]⁺ 363.22, found 363.4.

Step 3. To a solution of methyl 3-((4-((tert-butoxycarbonyl)amino)benzyl)(methyl)amino)cyclopentane-1-carboxylate (1.50 g, 4.14 mmol) in DCM (4 mL) was added TFA (4 mL) at 25° C. The mixture was stirred at 40° C. for 2 hr. The reaction mixture was concentrated directly The crude product was used in the next step directly Compound methyl 3-((4-aminobenzyl)(methyl)amino)cyclopentane-1-carboxylate (1.00 g, 3.81 mmol, 92% yield) was obtained as brown oil. LCMS Rt=0.148 min in 1 min chromatography, purity 65%, MS ESI calcd. for 262.17, [M+H]⁺ 263.17, found 263.0.

### Synthesis of methyl 2-pyrrolidin-3-ylpropanoate

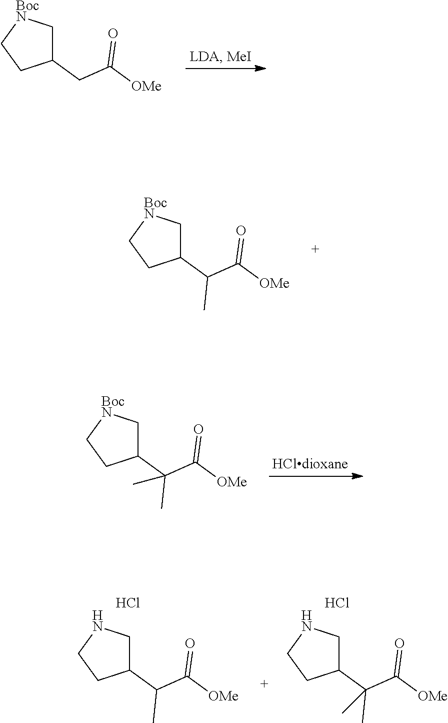

Step 1. To a stirred solution of tert-butyl 3-(2-methoxy-2-oxo-ethyl)pyrrolidine-1-carboxylate (400 mg, 1 64 mmol) in THF (5 mL) was added LDA (2M, 3 mL) slowly at −78° C. The mixture was stirred at −78° C. under N₂ for 1 hr. Then Mel (0.51 mL, 8.22 mmol) was added to the reaction mixture. The resulting mixture was stirred at 20° C. under N₂ for 1 hr. The reaction mixture was quenched with water (10 mL) and extracted with EtOAc (2×15 mL). The combine organic layers were washed with brine (20 mL), dried over anhydrous sodium sulfate, filtered and the filtrate was concentrated to afford a residue. The crude product was purified by flash chromatography on silica gel (0-100% EtOAc in PE) to afford a mixture of tert-butyl 3-(2-methoxy-1,1-dimethyl-2-oxo-ethyl)pyrrolidine-1-carboxylate (130 mg, 0.479 mmol, 29% yield) as a yellow oil and tert-butyl 3-(2-methoxy-1-methyl-2-oxo-ethyl)pyrrolidine-1-carboxylate (130 mg, 0.505 mmol, 30.7% yield) as yellow oil.

Step 2. To a solution of tert-butyl 3-(2-methoxy-1,1-dimethyl-2-oxo-ethyl)pyrrolidine-1-carboxylate (65.0 mg, 0.240 mmol), tert-butyl 3-(2-methoxy-1-methyl-2-oxo-ethyl)pyrrolidine-1-carboxylate (65.0 mg, 0.253 mmol) in 1,4-Dioxane (1 mL) was added HCl/dioxane (3 mL). The mixture was stirred at 25° C. for 16 hr. The mixture was concentrated to give a mixture of methyl 2-methyl-2-pyrrolidin-3-yl-propanoate (40 mg, crude) as a yellow solid and methyl 2-pyrrolidin-3-ylpropanoate (40.0 mg, crude) as a yellow solid.

### Synthesis of methyl trans-4-((1-(4-aminophenyl)ethyl)(methyl)amino)cyclohexane-1-carboxylate

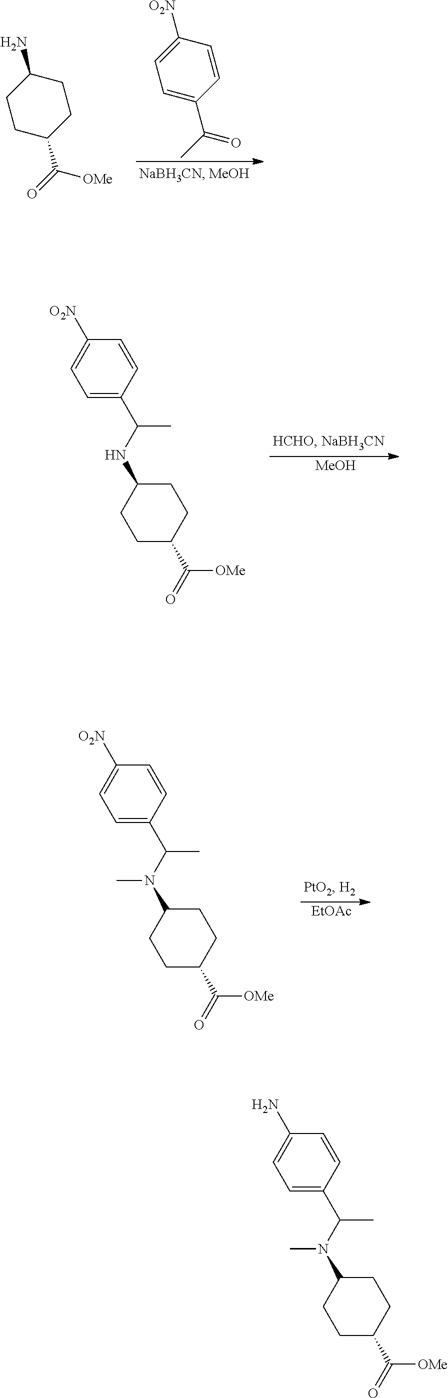

Step 1. To a solution of methyl trans-4-aminocyclohexane-1-carboxylate (5.00 g, 31.8 mmol) and 1-(4-nitrophenyl)ethan-1-one (6.30 g, 38.2 mmol) in Methanol (50 mL) was added NaCNBH₃ (9.99 g, 159 mmol) at 0° C. The mixture was stirred at 40° C. for 12 hrs. The reaction mixture was concentrated. Aq. NaHCO₃ (100 mL) was added. The resulting mixture was extracted with EtOAc (2×80 mL). The organic layer was dried over Na₂SO₄, filtered and concentrated to give methyl trans-4-((1-(4-nitrophenyl)ethyl)amino)cyclohexane-1-carboxylate (8.00 g, crude) as a yellow solid. LCMS Rₜ=0.448 min in 1 min chromatography, purity 47%, MS ESI calcd. for 306.16 [M+H]⁺ 307.16. found 307.3.

Step 2. To a solution of methyl trans-4-((1-(4-nitrophenyl)ethyl)amino)cyclohexane-1-carboxylate (0.50 g, 4.90 mmol) and HCHO (0.79 g, 9.79 mmol, 37% in water) in methanol (30.0 mL) was added NaBH₃CN (1.54 g, 24.5 mmol) at 0° C. The mixture was stirred at 40° C. for 12 hrs. The reaction was quenched with NaHCO₃ (50 mL) drop-wise. The resulting mixture was extracted with DCM (3×50 mL). The combined organic phase was washed with brine (20 mL), dried over anhydrous Na₂SO₄, filtered and concentrated. The residue was purified by flash silica gel chromatography (eluent of DCM @30 mL/min) and prep-HPLC (Column Welch Xtimate C18 250*50 mm*10 um; mobile phase: [water (FA)-ACN]; B %: 0%-35%; 20 min) to give methyl trans-4-(methyl(1-(4-nitrophenyl)ethyl)amino)cyclohexane-1-carboxylate (495 mg, 32% yield) as a yellow solid. ¹HNMR (400 MHz, DMSO-d₆) δ=8.18 (d, J=8.8 Hz, 2H), 7.62 (d, J=8.8 Hz, 2H), 3.98-3.89 (m, 1H), 3.55 (s, 3H), 2.25-2.16 (m, 1H), 2.10 (s, 3H), 1.93-1.87 (m, 2H), 1.77-1.66 (m, 2H), 1.44-1.30 (m, 3H), 1.28 (d, J=6.8 Hz, 3H), 1.25-1.21 (m, 2H).

Step 3. To a solution of methyl trans-4-(methyl(1-(4-nitrophenyl)ethyl)amino)cyclohexane-1-carboxylate (400 mg, 1.25 mmol) in ethyl acetate (10.0 mL) was added PtO₂ (240 mg) under Ar atmosphere. The suspension was degassed and purged with H₂ for 3 times. The reaction mixture was stirred under H₂ (15 Psi) at 25° C. for 2 hrs. The mixture was filtered through celite and washed with solvents (MeOH/DCM=1/5, 4×10 mL). The filtrate was concentrated to give methyl trans-4-((1-(4-aminophenyl)ethyl)(methyl)amino)cyclohexane-1-carboxylate (275 mg, 71% yield) as a yellow solid. ¹HNMR (400 MHz, DMSO-4) δ=6.93 (d, J=8.4 Hz, 2H), 6.48 (d, J=8.4 Hz, 2H), 4.85 (s, 2H), 3.58-3.51 (m, 4H), 2.45-2.40 (m, 1H), 2.22-2.12 (m, 1H), 2.03 (s, 3H), 1.90-1.84 (m, 2H), 1.72-1.56 (m, 2H), 1.37-1.21 (m, 4H), 1.18 (d, J=6.4 Hz, 3H).

### Synthesis of trans-4-((4-amino-3-methylbenzyl)(methyl)amino)cyclohexane-1-carboxylate

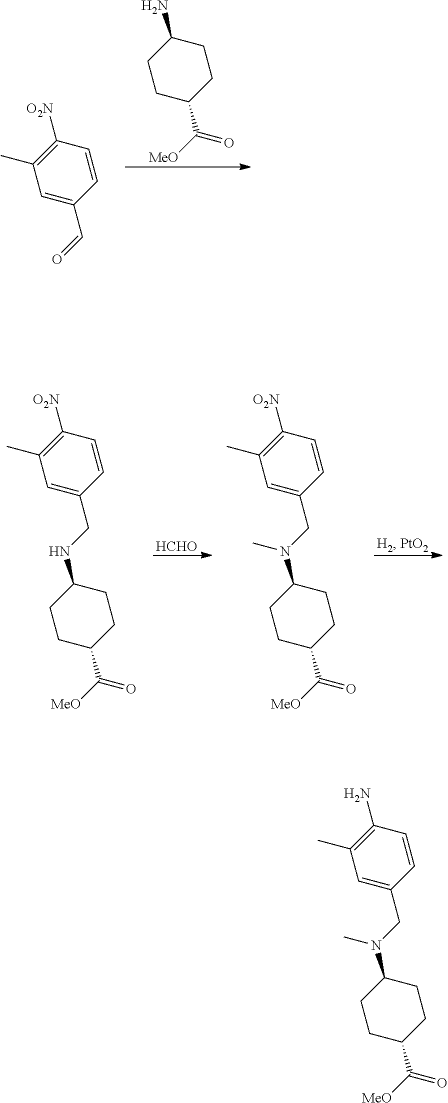

Step 1. To a solution of 3-methyl-4-nitrobenzaldehyde (500 mg, 3.03 mmol) in DCM (20.0 mL) was added methyl trans-4-aminocyclohexane-1-carboxylate (714 mg, 4.54 mmol) and NaBH(OAc) (1.28 g, 6.06 mmol) at 25° C. The mixture was stirred at 25° C. for 16 hrs. Sat. aq. NaHCO₃ (20 mL) was added to the residue. The resulting mixture was extracted with DCM (3×10 mL). The combined organic phase was washed with brine (10 mL), water (10 mL), dried over anhydrous Na₂SO₄, filtered and concentrated to give methyl trans-4-((3-methyl-4-nitrobenzyl)amino)cyclohexane-1-carboxylate (620 mg, 54% yield) as a light green solid, which was used in the next step directly. LCMS Rₜ=0.625 min in 1.5 min chromatography, purity 80.8%, MS ESI calcd. for 306.16, [M+H]⁺ 307.16, found 307.2.

Step 2. To a solution of methyl trans-4-((3-methyl-4-nitrobenzyl)amino)cyclohexane-1-carboxylate (620 mg, 2.02 mmol) in methanol (8.00 mL) was added formaldehyde (493 mg, 6.07 mmol) and NaBH₃CN (1.29 mg, 6.07 mmol) at 25° C. The mixture was stirred at 25° C. for 2 hrs. The reaction mixture was concentrated. Sat. aq. NaHCO₃ (20 mL) was added to the residue. The resulting mixture was extracted with DCM (3×10 mL). The combined organic phase was washed with brine (5 mL), water (5 mL), dried over anhydrous Na₂SO₄, filtered and concentrated to give methyl trans-4-(methyl(3-methyl-4-nitrobenzyl)amino)cyclohexane-1-carboxylate (700 mg, 92% yield) as a light yellow solid, which was used in the next step directly. LCMS Rₜ=0.623 min in 1.5 min chromatography, purity 82%, MS ESI calcd. for 320.17, [M+H]⁺ 321.17, found 321.2.

Step 3. To a solution of methyl trans-4-(methyl(3-methyl-4-nitrobenzyl)amino)cyclohexane-1-carboxylate (200 mg, 0.624 mmol) in ethyl acetate (6.00 mL) was added PtO₂ (70.0 mg) under Ar atmosphere. The suspension was degassed and purged with H₂ for 3 times. The reaction mixture was stirred under H₂ (15 Psi) at 25° C. for 3 hrs. The reaction mixture was filtered. The filter cake was washed with EtOAc (40 mL) and the filtrate was concentrated to give methyl trans-4-((4-amino-3-methylbenzyl)(methyl)amino)cyclohexane-1-carboxylate (260 mg, 93% yield) as colorless oil. LCMS Rₜ=0.470 min in 2 min chromatography, purity 91%, MS ESI calcd. for 290 20, [M+H]⁺ 291.20, found 291.2.

### Synthesis of methyl 2-(1-(4-amino-3-methylbenzyl)pyrrolidin-3-yl)-2-methylpropanoate

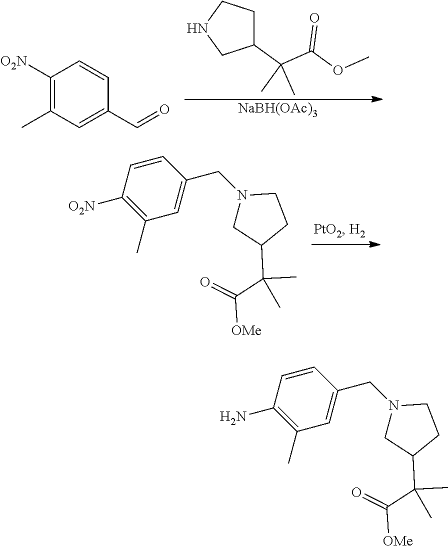

Step 1. A mixture of 3-methyl-4-nitrobenzaldehyde (300 mg, 1.82 mmol) and methyl 2-methyl-2-(pyrrolidin-3-yl)propanoate (342 mg, 2.00 mmol) and NaBH(OAc)₃ (1.15 g, 5.45 mmol) in DCE (4 mL) was degassed and purged with N₂ for 3 times, and then the mixture was stirred at 25° C. for 12 hrs under N₂ atmosphere. The reaction mixture was concentrated directly. The residue was purified by flash silica gel chromatography (eluent of 0~20% EtOAc in PE) to give methyl 2-methyl-2-(1-(3-methyl-4-nitrobenzyl)pyrrolidin-3-yl)propanoate (210 mg, 36% yield) as a white solid. LCMS Rₜ=0.372 min in 1 min chromatography, purity 98.5%, MS ESI calcd. for 320.17 [M+H]⁺ 321.17, found 321.1.

Step 2. To a solution of methyl methyl 2-methyl-2-(1-(3-methyl-4-nitrobenzyl)pyrrolidin-3-yl)propanoate (200 mg, 0.624 mmol) in THF (5 mL) was added PtO₂ (40.0 mg) under N₂ atmosphere. The suspension was degassed and purged with H₂ for 3 times. The reaction mixture was stirred under H₂ (15 Psi) at 25° C. for 1 hr. The reaction mixture was filtered. The filtrate was concentrated to give methyl 2-(1-(4-amino-3-methylbenzyl)pyrrolidin-3-yl)-2-methylpropanoate (190 mg, crude) as a yellow solid, which was used for the next step directly. LCMS Rₜ=0.318 min in 1 min chromatography, purity 98%, MS ESI calcd. for 290.20, [M+H]⁺ 291 20, found 291.1.

### Synthesis of tert-butyl 4-[2-(4-amino-2-fluoro-phenyl)ethyl]piperidine-1-carboxylate

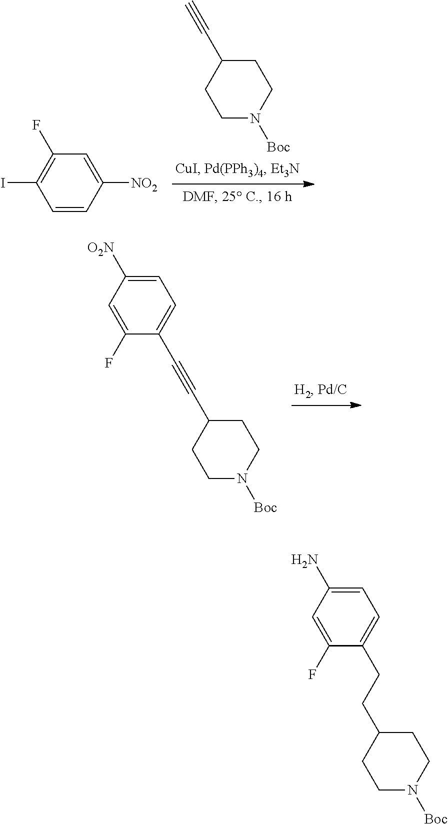

Step 1. To a solution of 2-fluoro-1-iodo-4-nitro-benzene (5.00 g, 18.7 mmol) in DMF (50 mL) was added TEA (26 mL, 187 mmol) at 25° C. under N₂. After stirring at 25° C. for 10 mins, tert-butyl 4-ethynylpiperidine-1-carboxylate (7.84 g, 37.5 mmol), Pd(PPh₃)₄ (0.65 g, 0.562 mmol) and CuI (178 mg, 0.936 mmol) was added to the mixture. The mixture was stirred at 25° C. for 16 hrs. H₂O (200 mL) was added. The mixture was extracted with EA (3×200 mL). The organic phase was washed with brine (200 mL), dried over Na₂SO₄, filtered and concentrated under reduce pressure to give a crude. The crude was purified by flash chromatography eluting with PE/EA (from 0% to 10% of EA) to give tert-butyl 4-[2-(2-fluoro-4-nitro-phenyl)ethynyl]piperidine-1-carboxylate (7.28 g, 20.9 mmol, crude) as a yellow oil. ¹H NMR (400 MHz, CHLOROFORM-d) 5=8.01-7.90 (m, 2H), 7.54 (dd, J=7.2, 8.4 Hz, 1H), 3.73 (ddd, J=3.6, 6.8, 13.2 Hz, 2H), 3.27 (ddd, J=3.6, 8.4, 13.6 Hz, 2H), 2.89 (tt, J=4.0, 7.6 Hz, 1H), 1.93-1.85 (m, 2H), 1.75-1.66 (m, 2H), 1.46 (s, 9H).

Step 2. To the solution of tert-butyl 4-[2-(2-fluoro-4-nitro-phenyl)ethynyl]piperidine-1-carboxylate (1.00 g, 2 87 mmol) in THF (10 mL) was added wet Pd/C (100 mg, 10%) at 25° C. The reaction was degassed and purged with Ar for 3 times, then degassed and purged with H₂ for 3 times and stirred under H₂ (50 Psi) for 16 h at 25° C. The mixture was concentrated under reduce pressure to give tert-butyl 4-[2-(4-amino-2-fluoro-phenyl)ethyl]piperidine-1-carboxylate (644 mg, 2.00 mmol, 70% yield) as a black oil. The crude was directedly used in the next step without purification. ¹H NMR (400 MHz, CHLOROFORM-d) δ=6.91 (t, J=8.0 Hz, 1H), 6.41-6.34 (m, 2H), 4.06 (d, J=1.6 Hz, 2H), 2.66 (t, J=12.0 Hz, 2H), 2.55-2.50 (m, 2H), 1.69 (d, J=12.4 Hz, 2H), 1.52-1.45 (m, 3H), 1.45 (s, 9H), 1 43-1.40 (m, 1H), 1.17-1.03 (m, 3H).

### Synthesis of methyl 1-((5-amino-6-methylpyridin-2-yl)carbamoyl)piperidine-4-carboxylate

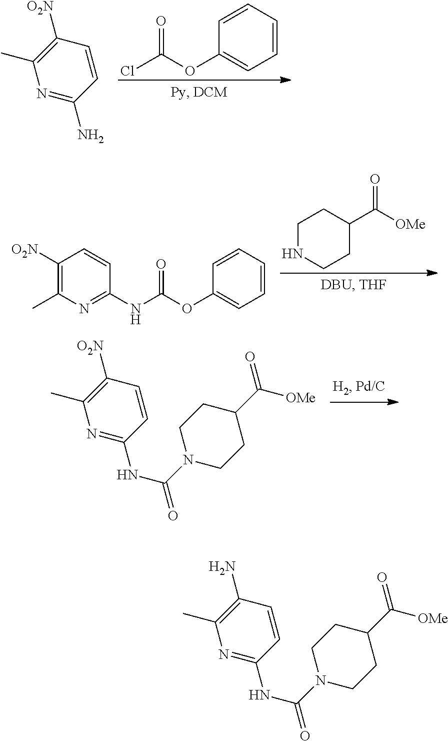

Step 1. To a solution of 6-methyl-5-nitro-pyridin-2-amine (2.00 g, 13.1 mmol) in DCM (20 mL) was added phenyl carbonochloridate (2.04 g, 13.1 mmol) and pyridine (1.03 g, 13.1 mmol) at 20° C. The mixture was stirred at 20° C. for 12 hrs. Water (20 mL) was added into the mixture The resulting mixture was extracted with DCM (3×20 mL). The combined organic phase was dried over anhydrous Na₂SO₄, filtered and concentrated to give phenyl (6-methyl-5-nitropyridin-2-yl)carbamate (3.50 g, 98% yield) as a light yellow solid. ¹HNMR (400 MHz, DMSO-d₆) δ=11.43 (s, 1H), 8.50 (d, J=9.2 Hz, 1H), 7.88 (d, J=9.2 Hz, 1H), 7.48-7.42 (m, 2H), 7.32-7.22 (m, 3H), 2.74 (s, 3H).

Step 2. To a solution of phenyl (6-methyl-5-nitropyridin-2-yl)carbamate (500 mg, 1.83 mmol) and methyl piperidine-4-carboxylate (393 mg, 2.74 mmol) in THF (5 mL) was added DBU (390 mg, 2.56 mmol). The mixture was stirred at 20° C. for 2 hrs. Water (10 mL) was added to the reaction mixture. The resulting mixture was extracted with EtOAc (3×10 mL). The combined organic phase was washed with brine (10 mL), dried over anhydrous Na₂SO₄, filtered and concentrated. The residue was purified by flash silica gel chromatography (eluent of 30-40% EtOAc in PE) to give methyl 1-((6-methyl-5-nitropyridin-2-yl)carbamoyl)piperidine-4-carboxylate (550 mg, 93% yield) as a light yellow solid. ¹HNMR (400 MHz, DMSO-d₆) δ=9.99 (s, 1H), 8.37 (d, J=9.2 Hz, 1H), 7.77 (d, J=9.2 Hz, 1H), 4.04 (d, J=13.6 Hz, 2H), 3.62 (s, 3H), 3.02-2.92 (m, 2H), 2.71 (s, 3H), 2.64-2.57 (m, 1H), 1.84 (dd, J=3.2, 13.2 Hz, 2H), 1.56-1.45 (m, 2H).

Step 3. To a solution of methyl 1-((6-methyl-5-nitropyridin-2-yl)carbamoyl)piperidine-4-carboxylate (550 mg, 1.71 mmol) in THF (10 mL) was added wet Pd/C (100 mg, 10%) under N₂. The reaction mixture was degassed and purged with H₂ (15 Psi) for 3 times The mixture was stirred at 25° C. for 4 hrs under H₂ (15 Psi). The reaction mixture was filtered and the filtrate was concentrated to give methyl 1-((5-amino-6-methylpyridin-2-yl)carbamoyl)piperidine-4-carboxylate (510 mg, crude) as a yellow solid, which was used for the next step directly. LCMS Rₜ=0.338 min in 1 min chromatography, purity 99%, MS ESI calcd. for 292.15 [M+H]⁺ 293.15, found 293.1.

### Synthesis of methyl trans-4-(((5-amino-6-methylpyridin-2-yl)methyl) (methyl)amino)cyclohexane-1-carboxylate

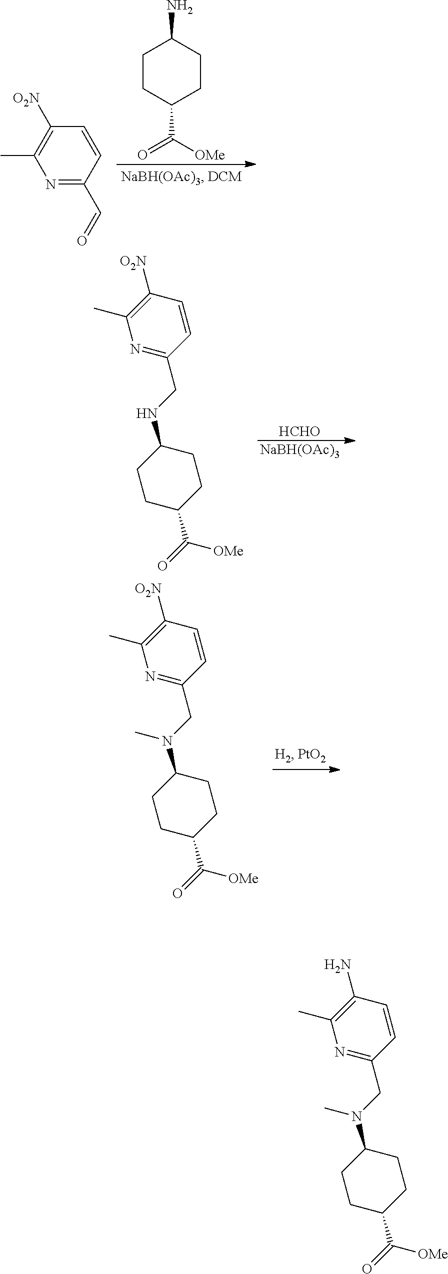

Step 1. To a solution of 6-methyl-5-nitropicolinaldehyde (0.8 g, 4.82 mmol) in DCM (20 mL) was added TEA (0.49 g, 4.82 mmol) methyl trans-4-aminocyclohexane-1-carboxylate (0.83 g, 5.30 mmol) and NaBH(OAc). (1.53 g, 7.22 mmol) at 25° C. The mixture was stirred at 25° C. for 2 hr. Water (10 mL) was added to the residue. The resulting mixture was extracted with DCM (3×10 mL). The combined organic phase was washed with brine (10 mL), dried over anhydrous Na₂SO₄, filtered and concentrated. The residue was purified by flash silica gel chromatography (eluent of 0-20% EtOAc in PE) to give methyl trans-4-(((6-methyl-5-nitropyridin-2-yl)methyl)amino)cyclohexane-1-carboxylate (1.40 g, 4.56 mmol) as light yellow oil. LCMS Rt=0.366 min in 1 min chromatography, purity 99% MS ESI calcd. for 307.15, [M+H]⁺ 308.15, found 308.1.

Step 2. To a solution of methyl trans-4-(((6-methyl-5-nitropyridin-2-yl)methyl)amino)cyclohexane-1-carboxylate (1.40 g, 4.56 mmol) in methanol (30 mL) was added formaldehyde (0.740 g, 9.11 mmol, 37% in water). The mixture was stirred at 25° C. for 0.5 hr. Then NaBH₃CN (572 mg, 9.11 mmol) was added into the mixture. The mixture was stirred at 25° C. for 12 hr. The reaction mixture was concentrated. Water (30 mL) was added to the residue. The resulting mixture was extracted with DCM (3×30 mL). The combined organic phase was washed with brine (30 mL), dried over anhydrous Na₂SO₄, filtered and concentrated. The residue was purified by flash silica gel chromatography (eluent of 030% EtOAc in PE) to give methyl trans-4-(methyl((6-methyl-5-nitropyridin-2-yl)methyl)amino)cyclohexane-1-carboxylate (1.05 g, 3.27 mmol) as yellow oil. ¹H NMR (DMSO-d₆, 400 MHz) δ=8.38 (d, J=8.4 Hz, 1H), 7.56 (d, J=8.4 Hz, 1H), 3.73 (s, 2H), 3.57 (s, 3H), 2.72 (s, 3H), 2.47-2.43 (m, 1H), 2.24-2.22 (m, 1H), 2.17 (s, 3H), 1.96-1.84 (m, 4H), 1.36-1.31 (m, 4H).

Step 3. To a solution of methyl (1r,4r)-4-(methyl ((6-methyl-5-nitropyridin-2-yl) methyl) amino) cyclohexane-1-carboxylate (1 g, 3 11 mmol) in ethanol (15 mL) and THF (15 mL) was added PtO₂ (350 mg) under N₂ atmosphere. The suspension was degassed and purged with H₂ for 3 times. The reaction mixture was stirred under H₂ (30 Psi) at 25° C. for 12 hr. The reaction mixture was filtered and the filtrate was concentrated to dryness. The residue was used in the next step directly. Compound methyl trans-4-(((5-amino-6-methylpyridin-2-yl)methyl) (methyl)amino)cyclohexane-1-carboxylate (900 mg, crude) as light yellow oil.

### Synthesis of methyl 2-(1-(4-amino-3-fluorophenyl)pyrrolidin-3-yl)-2-methylpropanoate

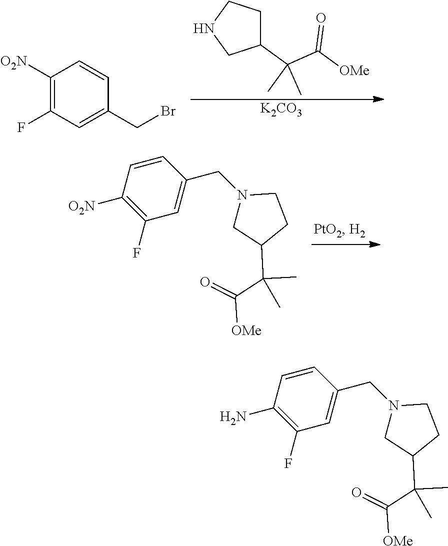

Step 1. To a solution of 4-(bromomethyl)-2-fluoro-1-nitrobenzene (450 mg, 1.92 mmol) in MeCN (5 mL) was added methyl 2-methyl-2-(pyrrolidin-3-yl)propanoate (329 mg, 1.92 mmol) and K₂CO₃ (797 mg, 5.77 mmol). The mixture was stirred at 60° C. for 12 hrs. Water (5 mL) was added to the reaction mixture. The reaction mixture was extracted with EtOAc (3×20 mL). The combined organic phase was washed with brine (5 mL), dried over anhydrous Na₂SO₄, filtered and concentrated. The residue was purified by flash silica gel chromatography (eluent of 0-20% EtOAc in PE) to give methyl 2-(1-(3-fluoro-4-nitrobenzyl)pyrrolidin-3-yl)-2-methylpropanoate (400 mg, 64% yield) as yellow oil. ¹H NMR (400 MHz, DMSO-d₆) δ=8.11 (t, J=8.4 Hz, 1H), 7.46 (d, J=12.4 Hz, 1H), 7.36 (d, J=8.4 Hz, 1H), 3.73-3.67 (m, 1H), 3.64-3 55 (m, 4H), 2.49-2.36 (m, 4H), 2.35-2.29 (m, 1H), 1.82-1.69 (m, 1H), 1.62-1.52 (m, 1H), 1.08 (s, 6H).

Step 2. To a solution of methyl 2-(1-(3-fluoro-4-nitrobenzyl)pyrrolidin-3-yl)-2-methylpropanoate (400 mg, 1.23 mmol) in ethyl acetate (5 mL) was added PtO₂ (0.1 g) under N₂ atmosphere. The suspension was degassed and purged with H₂ for 3 times. The reaction mixture was stirred under H₂ (15 Psi) at 25° C. for 1 hr. The reaction mixture was filtered and the filtrate was concentrate to give methyl 2 (1-(4-amino-3-fluorobenzyl)pyrrolidin-3-yl)-2-methylpropanoate (300 mg, 83% yield) as light yellow oil. LCMS Rₜ=0.339 min in 1.0 min chromatography, MS ESI calcd. for 294.17 [M-tBu+H]⁺ 295.17. found 295.2.

### Compounds Prepared in Accordance with General Scheme 5

The compounds herein (such as a compound of Formula I or Formula II, or other compounds in Section 1) may be prepared according to General Scheme 5, for example, from an intermediate as prepared in “Synthesis of additional intermediates for Table 3 compounds,” an intermediate that is commercially available, an intermediate as prepared elsewhere in this application or an intermediate that can be synthesized via a method known to those skilled in the art. An exemplary full synthesis is provided in Example 43 (Compound I-46). Compounds prepared in accordance with General Scheme 5 are provided in Table 3 below.

TABLE 3

| | Compound | | | |
|---|---|---|---|---|
| Example | No. | Structure | 1H NMR | LCMS |
| 149 | I-158 | 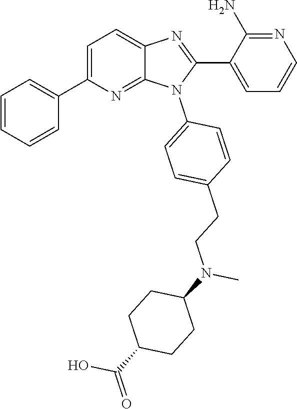 [SMILES: CN(CCc1ccc(-n2c(-c3cccnc3N)nc3ccc(-c4ccccc4)nc32)cc1)[C@H]1CC[C@H](C(=O)O)CC1] | ¹ H NMR (DMSO-d₆, 400 MHz) δ = 8.26 (d, J = 8.4 Hz, 1H), 8.05- 7.96 (m, 4H), 7.50- 7.43 (m, 2H), 7.42- 7.36 (m, 5H), 7.18 (dd, J = 1.6, 7.6 Hz, 1H), 7.01 (s, 2H), 6.38 (dd, J = 4.8, 7.6 Hz, 1H), 2.82-2.68 (m, 4H), 2.42- 2.36 (m, 1H), 2.28 (s, 3H), 2.14-2.04 (m, IH), 1.91 (d, J = 11.6 Hz, 2H), 1.73 (d, J = 11.6 Hz, 2H), 1.37- 1.17 (m, 4H). | MS ESI calcd. for 546.27 [M + H]⁺ 547.27, found 547.2 |
| 150 | I-159 | 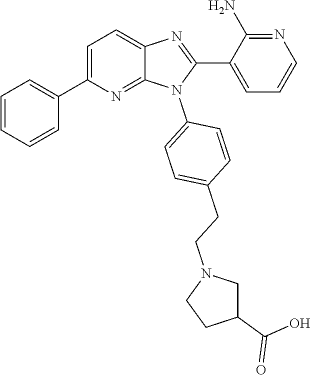 [SMILES: Nc1ncccc1-c1nc2ccc(-c3ccccc3)nc2n1-c1ccc(CCN2CCC(C(=O)O)C2)cc1] | ¹H NMR (DMSO-d₆, 400 MHz) δ = 8.26 (d, J = 8.4 Hz, 1H), 8.06- 7.93 (m, 4H), 7.51- 7.34 (m, 7H), 7.17 (dd, J= 1.6, 7.6 Hz, 1H), 6.99 (s, 2H), 6.40 (dd, J = 4.8, 7.6 Hz, 1H), 2.96-2.88 (m, 1H), 2.86- 2.78 (m, 3H), 2.73- 2.65 (m, 3H), 2.64- 2.52 (m, 2H), 1.95 (q, J = 7.2 Hz, 2H). | MS ESI calcd. for 504.23 [M + H]⁺ 505.23, found 505.2. |
| 151 | 1-160 | 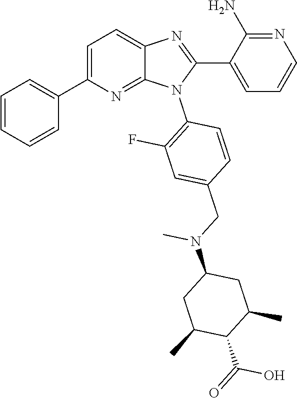 [SMILES: C[C@@H]1C[C@H](N(C)Cc2ccc(-n3c(-c4cccnc4N)nc4ccc(-c5ccccc5)nc43)c(F)c2)C[C@H](C)[C@H]1C(=O)O] | ¹H NMR (MeOD, 400 MHz,) δ =10.04-9.88 (m, 1H), 8.80-8.47 (m, 2H), 8.41 (d, J = 8.4 Hz, 1H), 8.19 (dd, J = 1.2, 6.0 Hz, 1H), 8.11 (d, J = 8.4 Hz, 1H), 8.05 (d, ) = 6.8 Hz, 2H), 7.95-7.87 (m, 2H), 7.86-7.80 (m, 1H), 7.74 (d, J = 8.0 Hz, 1H), 7.52-7.39 (m, 3H), 6.90-6.82 (m, 1H), 4.69-4.63 (m, 1H), 4.42 (d, J = 6.8 Hz, 1H), 3.48 (s, IH), 2.72 (d, J = 4.4 Hz, 3H), 2.38-2.28 (m, 1H), 2.20- 2.05 (m, 2H), 2.04- 1.91 (m, 1H), 1.66 (t, J = 10.6 Hz, 1H), 1.53- 1.37 (m, 2H), 0.89 (dd, J = 3.2, 6.0 Hz, 6H) | MS ESI calcd. For 578.28 [M + H]⁺ 579.28, found 579.2 |
| 152 | 1-161 | 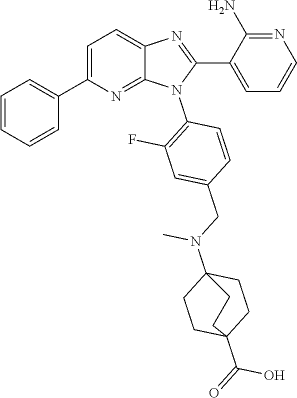 [SMILES: CN(Cc1ccc(-n2c(-c3cccnc3N)nc3ccc(-c4ccccc4)nc32)c(F)c1)C12CCC(C(=O)O)(CC1)CC2] | ¹H NMR (DMSO-d₆, 400 MHz) δ = 8.29 (d, J = 8.4 Hz, 1H), 8.05- 7.96 (m, 4H), 7.69- 7.62 (m, 1H), 7.49- 7.33 (m, 5H), 7.19- 7.10 (m, 3H), 6.39 (dd, J = 4.8, 7.6 Hz, 1H), 3.61 (s, 2H), 2.06 (s, 3H), 1.80-1.74 (m, 6H), 1.68-1.61 (m, 6H). | MS ESI calcd. for 576.26, [M + H]⁺ 577.26, found 577.3. |
| 153 | I-162 | 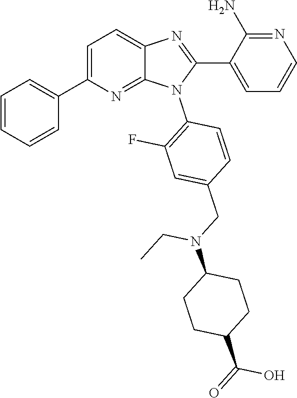 | ¹H NMR (400 MHz, DMSO-d₆) δ = 8.29 (d, J = 8.4 Hz, 1H), 8.04- 7.97 (m, 4H), 7.70- 7.61 (m, 1H), 7.49- 7.34 (m, 5H), 7.24- 7.13 (m, 3H), 6.36 (dd, J = 4.8, 7.8 Hz, 1H), 3.69 (s, 2H), 2.58-2.51 (m, 3H), 2.48 (s, 1H), 2.05 (d, J = 10.4 Hz, 2H), 1.66-1.57 (m, 2H), 1.53-1.35 (m, 4H), 0.97-0.90 (m, 3H). | MS ESI calcd. for 564.26 [M + H]⁺ 565.26 found 565.3. |
| 154 | I-163 | 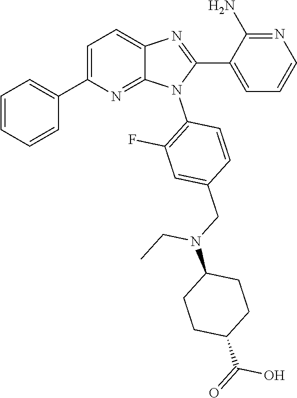 [SMILES: CCN(Cc1ccc(-n2c(-c3cccnc3N)nc3ccc(-c4ccccc4)nc32)c(F)c1)[C@H]1CC[C@H](C(=O)O)CC1] | ¹H NMR (DMSO-d₆, 400 MHz) δ = 8.29 (d, J = 8.4 Hz, 1H), 8.09- 7.88 (m, 4H), 7.72- 7.59 (m, 1H), 7.53- 7.29 (m, 5H), 7.27- 7.08 (m, 3H), 6.35 (dd, J = 4.8, 7.6 Hz, 1H), 3.71 (s, 2H), 2.61-2.51 (m, 3H), 2.11 (d, J = 8.4 Hz, 1H), 1.94 (d, J = 8.0 Hz, 2H), 1.85-1.74 (m, 2H), 1.39-1.21 (m, 4H), 0.95 (t, J = 7.2 Hz, 3H) | MS ESI caled. for 564.26 [M + H]⁺ 565.26, found 565.3 |
| 155 | I-164 | 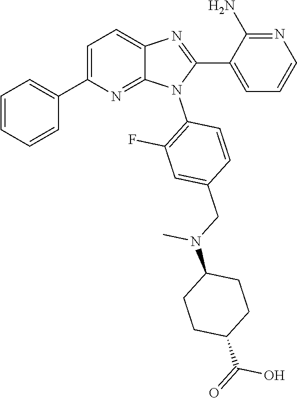 [SMILES: CN(Cc1ccc(-n2c(-c3cccnc3N)nc3ccc(-c4ccccc4)nc32)c(F)c1)[C@H]1CC[C@H](C(=O)O)CC1] | 1H NMR (400 MHz, DMSO-d₆) δ = 12.01 (s, 1H), 8.35-8.25 (m, 1H), 8.10-7.95 (m, 4H), 7.68 (t, J = 7.6 Hz, 1H), 7.55-7.45 (m, 2H), 7.44-7.35 (m, 3H), 7.18-7.05 (m, 3H), 6.39 (dd, J = 7.6, 4.8 Hz, 1H), 3.32 (s, 2H), 2.51-2.49 (m, 1H), 2.43 (s, 3H), 2.18- 2.14 (m, 1H), 1.99-1.83 (m, 4H), 1,38-1.30 (m, 3H | MS ESI calcd. For 550.25 [M + H]⁺ 551.25, found 551.2. |
| 156 | I-165 | 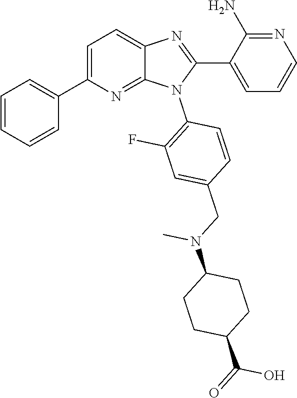 [SMILES: CN(Cc1ccc(-n2c(-c3cccnc3N)nc3ccc(-c4ccccc4)nc32)c(F)c1)[C@H]1CC[C@@H](C(=O)O)CC1] | ¹H NMR (400 MHz, DMSO-d₆) δ = 8.29 (d, J = 8.4 Hz, IH), 8.15 (s, 1H), 8.04-7.98 (m, 4H), 7.71-7.64 (m, 1H), 7.50-7.43 (m, 2H), 7.42-7.35 (m, 3H), 7.19-7.12 (m, 3H), 6.39 (dd, J = 4.8, 8.0 Hz, 1H), 3.65 (s, 2H), 2.44 (d, J = 5.6 Hz, 1H), 2.14 (s, 3H), 2.08- 2.00 (m, 2H), 1.67- 1.43 (m, 6H). | MS ESI calcd. for 550.25 [M + H]⁺ 551.25, found 551.3 |
| 157 | 1-166 | 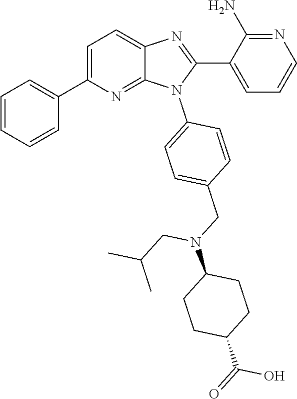 [SMILES: CC(C)CN(Cc1ccc(-n2c(-c3cccnc3N)nc3ccc(-c4ccccc4)nc32)cc1)[C@H]1CC[C@H](C(=O)O)CC1] | ¹HNMR (400 MHz, DMSO-d₆) δ = 12.35- 11.83 (m, 1H), 8.26 (d, J = 8.4 Hz, IH), 8.14 (s, 1H), 8.06-7.93 (m, 4H), 7.53-7.35 (m, 7H), 7.15 (s, 3H), 6.32 (t, J = 6.0 Hz, 1H), 3.65 (s, 2H), 2.41 (t, J = 10.4 Hz, 1H), 2.23 (d, J = 6.8 Hz, 2H), 2.17-2.07 (m, 1H), 2.00-1.76 (m, 4H), 1.58 (td, J = 6.8, 13.2 Hz, 1H), 1.40- 1.21 (m, 4H), 0.79 (d, J = 6.4 Hz, 6H). | MS ESI calcd. for 574.31, [M + H]⁺ 575.31, found 575.2 |
| 158 | I-167 | 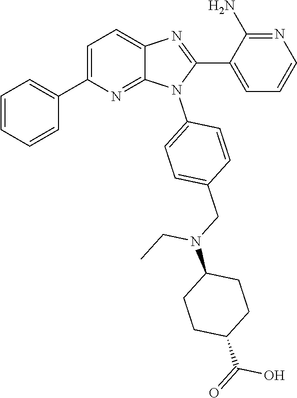 [SMILES: CCN(Cc1ccc(-n2c(-c3cccnc3N)nc3ccc(-c4ccccc4)nc32)cc1)[C@H]1CC[C@H](C(=O)O)CC1] | ¹HNMR (400 MHz, DMSO-d₆) δ = 8.27 (d, J = 8.4 Hz, 1H), 8.16 (s, IH), 8.06-7.96 (m, 4H), 7.57-7.33 (m, 7H), 7.19-7.07 (m, 3H), 6.34 (dd, J = 4.8, 7.6 Hz, 1H), 3.71 (s, 2H), 2.56 (q, ) = 7.2 Hz, 2H), 2.18-2.10 (m, 1H), 1.99-1.78 (m, 4H), 1.42-1.23 (m, 4H), 0.97 (t, J = 7.2 Hz, 3H). | MS ESI calcd. for 546.27, [M + H]⁺ 547.27, found 547.2. |
| 159 | I-168 | 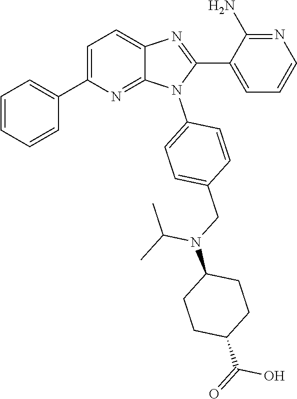 [SMILES: CC(C)N(Cc1ccc(-n2c(-c3cccnc3N)nc3ccc(-c4ccccc4)nc32)cc1)[C@H]1CC[C@H](C(=O)O)CC1] | ¹HNMR (400 MHz, DMSO-d₆) δ = 8.25 (d. J = 8.4 Hz, 1H), 8.04- 7.93 (m, 4H), 7.56- 7.33 (m, 7H), 7.13 (d, J = 6.4 Hz, 1H), 6.33 (dd, J = 4.8, 7.6 Hz, 1H), 3.74 (s, 2H), 3.02 (td, J = 6.4, 12.8 Hz, 1H), 2.49-2.47 (m, 1H), 2.08-2.01 (m, IH), 1.92-1.70 (m, 4H), 1.36-1.20 (m, 4H), 1.01 (d, J = 6.4 Hz, 6H). | MS ESI calcd. for 560.29, [M + H]⁺ 561.29, found 561.1 |
| 160 | I-169 | 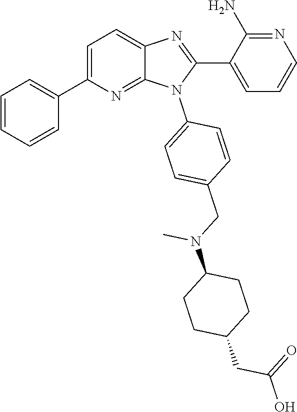 [SMILES: CN(Cc1ccc(-n2c(-c3cccnc3N)nc3ccc(-c4ccccc4)nc32)cc1)[C@H]1CC[C@H](CC(=O)O)CC1] | ¹HNMR (400 MHz, DMSO-d₆) δ = 8.27 (d, J = 8.4 Hz, IH), 8.07- 7.93 (m, 4H), 7.50- 7.35 (m, 7H), 7.18- 7.13 (m, 1H), 7.03 (s, 2H), 6.37 (dd, ) = 4.8, 7.2 Hz, 1H), 3.63 (s, 2H), 2.46-2.36 (m, 2H), 2.15 (s, 3H), 2.09 (d, J = 7.2 Hz, 2H), 1.87- 1.74 (m, 4H), 1.66- 1.52 (m, 1H), 1.43- 1.24 (m, 2H), 1.05- 0.88 (m, 2H). | MS ESI calcd. for 546.27 [M + H]⁺ 547.27, found 547.3. |
| 161 | I-170 | 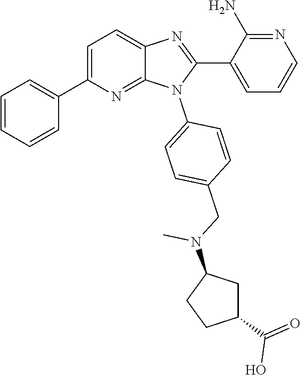 [SMILES: CN(Cc1ccc(-n2c(-c3cccnc3N)nc3ccc(-c4ccccc4)nc32)cc1)[C@@H]1CC[C@@H](C(=O)O)C1] | .¹HNMR (400 MHz, DMSO-d₆) δ = 8.24 (d, J = 8.4 Hz, 1H), 8.04- 7.92 (m, 4H), 7.52- 7.33 (m, 7H), 7.15 (dd, J = 7.6, 1.2 Hz, 1H), 6.38 (dd, J = 7.6, 4.8 Hz, 1H), 3.55 (s, 2H), 2.92-2.82 (m, 1H), 2.82-2.72 (m, 1H), 2.06 (s, 3H), 2.02-1.84 (m, 3H), 1.79-1.59 (m, 2H), 1.54-1.41 (m, 1H). | MS ESI calcd. for 518.24, [M + H]⁺ 519.24, found 519.3 |
| 162 | I-171 | 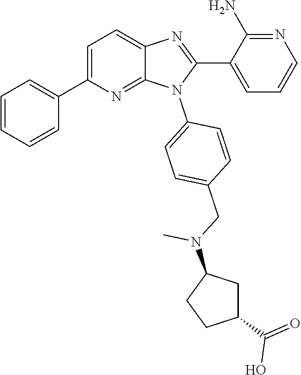 [SMILES: CN(Cc1ccc(-n2c(-c3cccnc3N)nc3ccc(-c4ccccc4)nc32)cc1)[C@@H]1CC[C@@H](C(=O)O)C1] | ¹HNMR (400 MHz, DMSO-d₆) δ = 8.27 (d. J = 8.4 Hz, 1H), 8.07- 7.96 (m, 4H), 7.52- 7.35 (m, 7H), 7.15 (dd, J = 7.6, 1.6 Hz, 1H), 7.03 (s, 2H), 6.38 (dd, J = 7.6, 4.8 Hz, 1H), 3.58 (s, 2H), 2.90-2.80 (m, 1H), 2.76-2.65 (m, 1H), 2.16-2.05 (m, 4H), 1.89-1.77 (m, 3H), 1.73-1.52 (m, 2H) | MS ES! calcd. for 518.24, [M + H]⁺ 519.24, found 519.3. |
| 163 | I-172 | 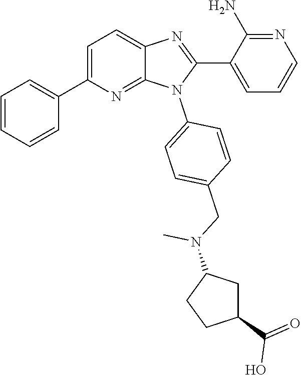 | . ¹HINMR (400 MHz, DMSO-d₆) δ = 8.27 (d, J= 8.4 Hz, IH), 8.06- 7.96 (m, 4H), 7.50- 7.36 (m, 7H), 7.15 (dd, J = 7.6, 1.6 Hz, 1H), 7.07-6.99 (m, 2H), 6.38 (dd, J = 7.6, 4.8 Hz, 1H), 3.56 (s, 2H), 2.92-2.86 (m, 1H), 2.79 (dd, J = 8.8, 5.6 Hz, 1H), 2.10-1.99 (m, 4H), 1.97-1.86 (m, 2H), 1.80-1.64 (m, 2H), 1.53-1.43 (m, 1H) | MS ESI calcd. for 518.24, [M + H]⁺ 519.24, found 519.4. |
| 164 | 1-173 | 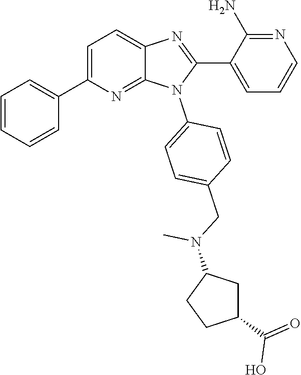 | ¹HINMR (400 MHz, DMSO-d₆) δ = 8.27 (d, J = 8.4 Hz, 1H), 8.05- 7.96 (m, 4H), 7.50- 7.36 (m, 7H), 7.15 (dd, J = 7.6, 1.6 Hz, 1H), 7.03 (s, 2H), 6.38 (dd, J = 7.6, 4.8 Hz, 1H), 3.58 (s, 2H), 2.87-2.81 (m, 1H), 2.74-2.68 (m, 1H), 2.15-2.05 (m, 4H), 1.88-1.76 (m, 3H), 1.73-1.52 (m, 2H). | MS ESI calcd. for 518.24, [M + H]⁺ 519.24, found 519.3 |
| 165 | I-174 | 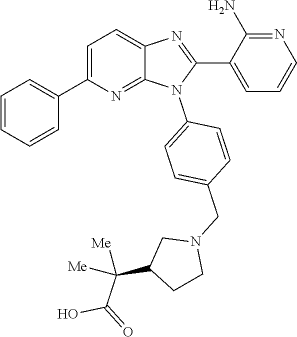 | ¹HNMR (400 MHz, DMSO-d₆) δ = 8.26 (d. J = 8.4 Hz, 1H), 8.05- 7.96 (m, 4H), 7.50- 7.36 (m, 7H), 7.14 (dd, J = 1.6, 7.6 Hz, 1H), 7.04 (s, 2H), 6.38 (dd, J = 4.8, 7.6 Hz, 1H), 3.71 (d. ] = 13.2 Hz, 1H), 3.57 (d, J = 13.2 Hz, 1H), 2.54 (s, 2H), 2.44 (dd, J = 7.2, 15.6 Hz. 2H), 2.36-2.30 (m, 1H), 1.84-1.73 (m, 1H), 1.64-1.53 (m, 1H), 1.05 (s, 6H) | MS ESI calcd. for 532.26 [M + H]⁺ 533.26, found 533.3. |
| 166 | 1-175 | 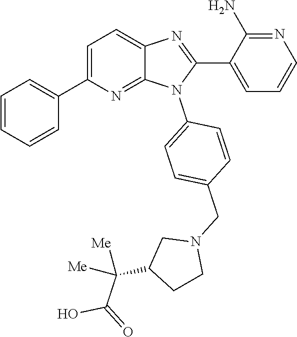 | ¹HNMR (400 MHz, DMSO-d₆) δ = 8.26 (d, J = 8.4 Hz, IH), 8.07- 7.96 (m, 4H), 7.52- 7.35 (m, 7H), 7.15 (d, J = 7.6 Hz, 1H), 7.04 (s, 2H), 6.38 (dd, ) = 4.8, 7.2 Hz, 1H), 3.71 (d, J = 13.2 Hz, 1H), 3.57 (d, J = 13.2 Hz, 1H), 2.54 (s, 2H), 2.49-2.39 (m, 2H), 2.37-2.30 (m, 1H), 1.84-1,72 (m, 1H), 1.65-1.53 (m, 1H), 1.05 (s, 6H) | MS ESI calcd. for 532.26 [M + H]⁺ 533.26, found 533.3. |
| 167 | I-176 | 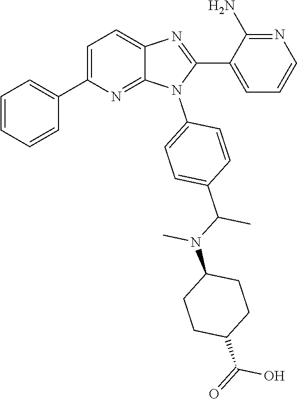 | ¹HNMR (DMSO-d₆, 400 MHz) δ = 11.24- 10.59 (m, 1H), 8.53- 8.28 (m, 2H), 8.23- 7.90 (m, 6H), 7.88- 7.76 (m, 1H), 7.76- 7.61 (m, 2H), 7.54- 7.34 (m, 3H), 6.92- 6.75 (m, 1H), 4.77- 4.65 (m, 1H), 2.85- 2.61 (m, 3H), 2.39 (d, J = 4.8 Hz, 1H), 2.23- 2.17 (m, 1H), 2.06- 1.85 (m, 3H), 1.83 ~ 1.71 (m, 3H), 1.70- 1.39 (m, 3H), 1.26- 1.10 (m, 1H), 1.01- 0.84 (m, 1H). | MS ESI calcd. for 546.27 [M + H]⁺ 547.27, found 547.1. |
| 168 | I-177 | 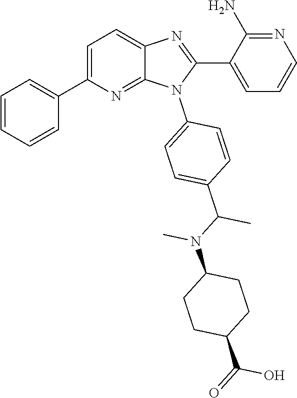 [SMILES: CC(c1ccc(-n2c(-c3cccnc3N)nc3ccc(-c4ccccc4)nc32)cc1)N(C)[C@H]1CC[C@@H](C(=O)O)CC1] | ¹HNMR (DMSO-d₆, 400 MHz) δ = 8.27 (d, J = 8.4 Hz, 1H), 8.05- 7.95 (m, 4H), 7.54- 7.49 (m, 2H), 7.49- 7.36 (m, 5H), 7.19- 7.04 (m, 3H), 6.38- 6.30 (m, 1H), 3,99- 3.80 (m, 1H), 2.47- 2.38 (m, 1H), 2.16- 2.05 (m, 3H), 2.04- 1.87 (m, 2H), 1.77- 1.10 (m, 10H). | MS ESI calcd. for 546.27 [M + H]⁺ 547.27, found 547.1. |
| 169 | I-178 | 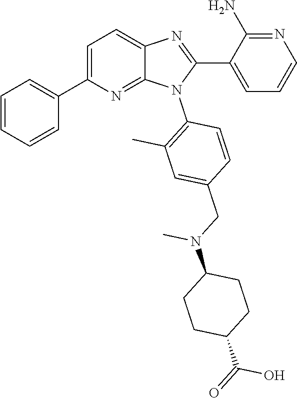 [SMILES: Cc1cc(CN(C)[C@H]2CC[C@H](C(=O)O)CC2)ccc1-n1c(-c2cccnc2N)nc2ccc(-c3ccccc3)nc21] | ¹H NMR (400 MHz, DMSO-d₆) δ_{H} = 8.28 (d, J = 8.4 Hz, 1H), 8.01- 7.94 (m, 4H), 7.48- 7.42 (m, 2H), 7.41- 7.33 (m, 4H), 7.33- 7.29 (m, 2H), 7.02 (dd, J = 1.6, 7.6 Hz, 1H), 6.31 (dd, J= 4.8, 7.6 Hz, 1H), 3.62 (s, 2H), 2.46-2.39 (m, 1H), 2.16 (s, 4H), 2.00-1.92 (m, 5H), 1.89-1.80 (m, 2H), 1.34 (d, ) = 10.4 Hz, 4H). | %, MS ESI calcd. for 546.27, [M + H]⁺ 547.27, found 547.1. |
| 170 | I-179 | 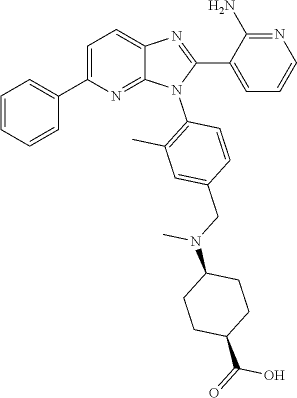 [SMILES: Cc1cc(CN(C)[C@H]2CC[C@@H](C(=O)O)CC2)ccc1-n1c(-c2cccnc2N)nc2ccc(-c3ccccc3)nc21] | ¹H NMR (DMSO-d6. 400 MHz) δ = 8.29 (d, J = 8.4 Hz, 1H), 8.01- 7.95 (m, 4H), 7.49- 7.43 (m, 2H), 7.42- 7.34 (m, 4H), 7.32 (s, 2H), 7.02 (dd, J = 2.0, 8.0 Hz, 1H), 6.32 (dd, J = 4.8, 7.6 Hz, 1H), 3.60 (s, 2H), 2.47-2.40 (m, 1H), 2.20-1.97 (m, 6H), 1.96 (s, 3H), 1.71- 1.62 (m, 2H), 1.60- 1.41 (m, 4H). | MS ESI calcd. for 546.27 [M + H]⁺ 547.27, found 547.3. |
| 171 | I-180 | 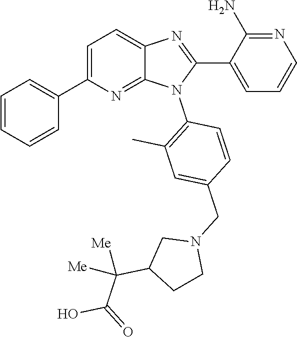 [SMILES: Cc1cc(CN2CCC(C(C)(C)C(=O)O)C2)ccc1-n1c(-c2cccnc2N)nc2ccc(-c3ccccc3)nc21] | ¹H NMR (400 MHz, DMSO-d₆) δ = 8.28 (d. J = 8.4 Hz, 1H), 8.02- 7.95 (m, 4H), 7.49- 7.42 (m, 2H), 7.41- 7.33 (m, 4H), 7.30 (s, 2H), 7.01 (d, J = 6.4 Hz, 1H), 6.40-6.31 (m, 1H), 3.63-3.54 (m, 2H), 2.58-2.53 (m, 2H), 2.47-2.38 (m, 1H), 2.37-2.31 (m, 1H), 2.28-2.20 (m, 1H), 1.94 (d, J = 1.6 Hz, 3H), 1.71-1.50 (m, 2H), 0.88 (s, 6H). | MS ESI calcd. for 546.27 [M + H]⁺ 547.27, found 547.3. |
| 172 | I-181 | 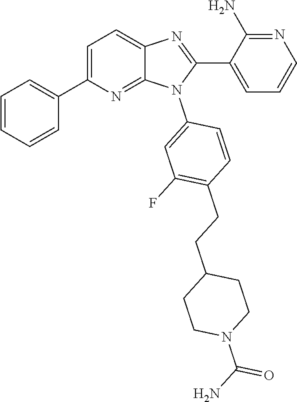 [SMILES: NC(=O)N1CCC(CCc2ccc(-n3c(-c4cccnc4N)nc4ccc(-c5ccccc5)nc43)cc2F)CC1] | ¹H NMR (400 MHz, DMSO-d₆) δ = 8.27 (d, J = 8.0 Hz, IH), 8.06- 7.97 (m, 4H), 7.50- 7.38 (m, 5H), 7.24 (ddd, J = 1.6, 8.0, 12.8 Hz, 2H), 6.90 (s, 2H), 6.46 (dd, J = 5.2, 7.6 Hz, 1H), 5.85 (s, 2H), 3.93 (d, J = 13.2 Hz, 2H), 2.76-2.68 (m, 2H), 2.65-2.56 (m, 2H), 1.68 (br d, J = 11.2 Hz, 2H), 1.60-1.52 (m, 2H), 1.41 (dt, J = 3.2, 7.2 Hz, 1H), 1.03 (dq, J = 4.0, 12.0 Hz, 2H). | MS ESI calcd. for 535.63 [M + H]⁺ 536.63, found 536.4. |
| 173 | I-182 | 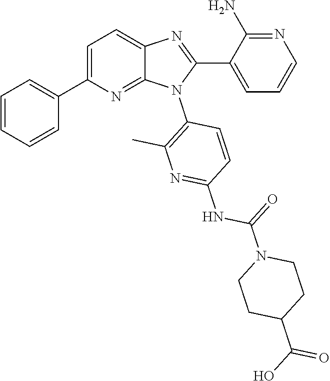 [SMILES: Cc1nc(NC(=O)N2CCC(C(=O)O)CC2)ccc1-n1c(-c2cccnc2N)nc2ccc(-c3ccccc3)nc21] | ¹HNMR (400 MHz, DMSO-d₆) δ = 9.55- 9.31 (m, 1H), 8.29 (d, J = 8.4 Hz, IH), 8.05- 7.97 (m, 4H), 7.83- 7.73 (m, 2H), 7.50- 7.36 (m, 3H), 7.24- 7.17 (m, 3H), 6.46 (dd, J = 4.8, 7.6 Hz, 1H), 4.05 (d, J = 13.6 Hz, 2H), 2.95 (t, J = 11.2 Hz, 2H), 2.41 (t, J = 10.4 Hz, 1H), 2.04 (s, 3H), 1.85-1.77 (m. 2H), 1.55-1.44 (m, 2H). | MS ESI calcd. for 548.23, [M + H]⁺ 549.23, found 549.2. |
| 174 | I-183 | 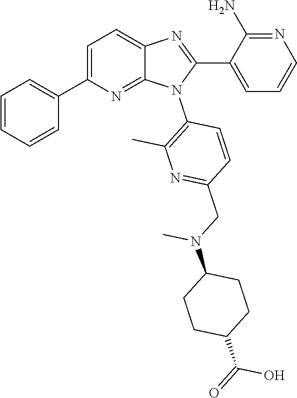 | ¹H NMR (DMSO-d₆, 400 MHz) δ = 8.31 (d, J = 8.4 Hz, 1H), 8.04- 7.95 (m, 4H), 7.82 (d, J = 8.0 Hz, 1H), 7.50- 7.36 (m, 4H), 7.18 (s, 2H), 7.11-7.04 (m, 1H), 6.38 (dd, J = 4.8, 7.6 Hz, 1H), 3.81-3.69 (m, 2H), 2.47-2.39 (m, 1H), 2.21 (s, 3H), 2.14 (s, 4H), 1.99-1.82 (m, 4H), 1.40-1.27 (m, 4H | MS ESI calcd. for 547.27, [M + H]⁺ 548.27, found 548.3. |
| 175 | I-184 | 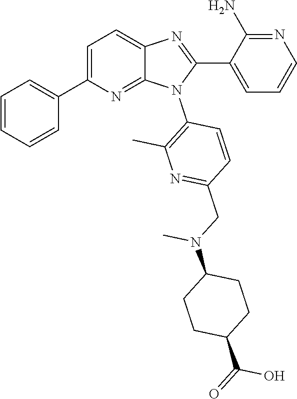 [SMILES: Cc1nc(CN(C)[C@H]2CC[C@@H](C(=O)O)CC2)ccc1-n1c(-c2cccnc2N)nc2ccc(-c3ccccc3)nc21] | ¹H NMR (DMSO-d₆, 400 MHz) δ = 12.43- 11.67 (m, 1H), 8.31 (d, J = 8.4 Hz, 1H), 8.05- 7.94 (m, 4H), 7.84 (d, J = 8.4 Hz, 1H), 7.51- 7.36 (m, 4H), 7.19 (s, 2H), 7.10-7.04 (m. 1H), 6.38 (dd, J = 4.8, 7.6 Hz, 1H), 3.80-3.66 (m, 2H), 2.49-2.39 (m, 2H), 2.18 (s, 3H), 2.14 (s, 3H), 2.07-1.99 (m, 2H), 1.67-1.41 (m, 6H). | MS ESI calcd. for 547.27, [M + H]⁺ 548.27, found 548.2. |
| 176 | I-185 | 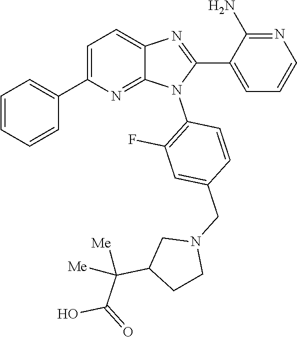 [SMILES: CC(C)(C(=O)O)C1CCN(Cc2ccc(-n3c(-c4cccnc4N)nc4ccc(-c5ccccc5)nc43)c(F)c2)C1] | ¹H NMR (400 MHz, DMSO-d₆) δ = 8.30 (d, J = 8.4 Hz, 1H), 8.07- 7.96 (m, 4H), 7.72- 7.62 (m, 1H), 7.52- 7.33 (m, 5H), 7.20- 7.10 (m, 3H), 6.41 (dd, J = 4.8, 7.6 Hz, 1H), 3.63-3.57 (m, 2H), 2.59-2.53 (m, 2H), 2.49-2.39 (m, 2H), 2.38-2.28 (m, 1H), 1.83-1.69 (m, 1H), 1.65-1.55 (m, IH). 1.01 (s, 6H). | MS ESI calcd. for 550.25 [M + H]⁺ 551.25, found 551.2 |

### Example 177. Synthesis of trans-4-((4-(2-(2-aminopyridin-3-yl)-5-phenyl-3H-imidazo[4,5-b]pyridin-3-yl)benzyl)(methyl)amino)-N-methylcyclohexane-1-carboxamide (Compound I-186)

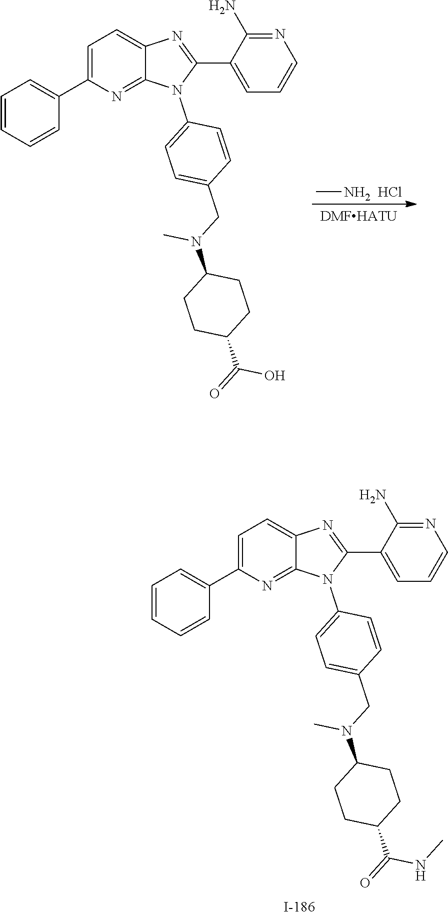

Step 1. To a solution of trans-4-((4-(2-(2-aminopyridin-3-yl)-5-phenyl-3H-imidazo[4,5-b]pyridin-3-yl)benzyl)(methyl)amino)cyclohexane-1-carboxylic acid (280 mg, 0.526 mmol) and DIEA (0.27 mL, 1.58 mmol) in DMF (5 mL) was added HATU (300 mg, 0.789 mmol) slowly at 0° C. for 0.5 hr. Then MeNH₂·HCl (106 mg, 1.58 mmol) was added dropwise at 25° C. The resulting mixture was stirred at 25′C for 16 hr. The reaction mixture was filtered with MeOH (3 mL) and DMF (3 mL). The reaction mixture was filtered. The combined organic phase was purified by prep-HPLC (column: Welch Xtimate C18 40*200 mm 7 um; mobile phase: water (NH₄HCO₃)-ACN; gradient:28%-68% B over 14 min) to give trans-4-((4-(2-(2-aminopyridin-3-yl)-5-phenyl-3H-imidazo[4,5-b]pyridin-3-yl)benzyl)(methyl)amino)-N-methylcyclohexane-1-carboxamide 1-186 (194 mg, 0.354 mmol, 67% yield) as a light yellow solid. ¹HNMR (400 MHz, DMSO-d₆) δ_{H}=8.26 (d, J=8.4 Hz, 1H), 8.07-7.94 (m, 4H), 7.64 (q, J=4.0 Hz, 1H), 7.50-7.37 (m, 7H), 7.15 (dd, J=1.6, 7.6 Hz, 1H), 7.04 (s, 2H), 6.39 (dd, J=4.8, 7.6 Hz, 1H), 3.64 (s, 2H), 2.55 (d, J=4.4 Hz, 3H), 2.46-2.38 (m, 1H), 2.15 (s, 3H), 2.07-1.97 (m, 1H), 1.88-1.75 (m, 4H), 1.41-1.25 (m, 4H). HPLC Rₜ=3.819 min in 8 min chromatography, purity 99.6%. LCMS Rₜ=1.416 min in 4 min chromatography, purity 99.4%, MS ESI calcd. For 545.29, [M+H]⁺ 546.29, found 546.3.

### Example 178. Synthesis of trans-4-((4-(2-(2-aminopyridin-3-yl)-5-phenyl-3H-imidazo[4,5-b]pyridin-3-yl)benzyl)(methyl)amino)cyclohexane-1-carboxamide (Compound I-187)

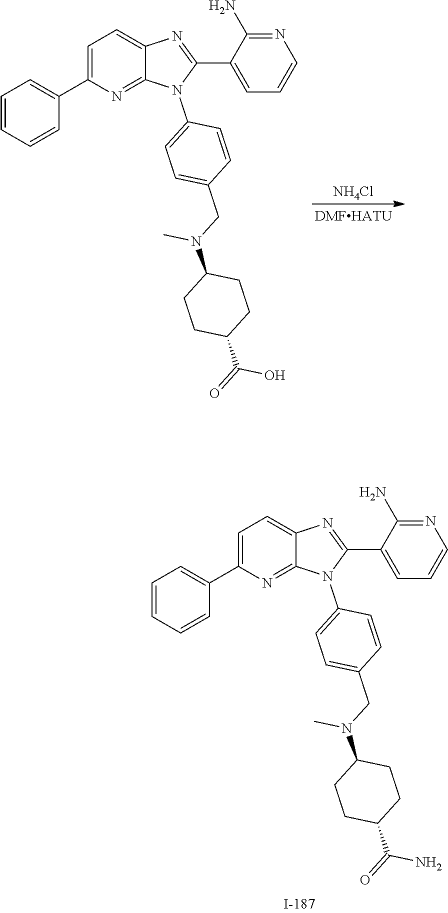 [SMILES: CN(Cc1ccc(-n2c(-c3cccnc3N)nc3ccc(-c4ccccc4)nc32)cc1)[C@H]1CC[C@H](C(=O)O)CC1 ; "NH4Cl" ; CN(Cc1ccc(-n2c(-c3cccnc3N)nc3ccc(-c4ccccc4)nc32)cc1)[C@H]1CC[C@H](C(N)=O)CC1]

Step 1. To a solution of trans-4-((4-(2-(2-aminopyridin-3-yl)-5-phenyl-3H-imidazo[4,5-b]pyridin-3-yl)benzyl)(methyl)amino)cyclohexane-1-carboxylic acid (280 mg. 0.526 mmol) and DIEA (0.27 mL, 1.58 mmol) in DMF (5 mL) was added HATU (300 mg, 0.789 mmol) slowly at 0° C. for 0.5 hr. Then NH₄Cl (84.0 mg, 1.58 mmol) was added dropwise at 25° C. The resulting mixture was stirred at 25° C. for 16 hr. The reaction mixture was filtered with MeOH (3 mL) and DMF (3 mL). The reaction mixture was filtered. The combined organic phase was purified by prep-HPLC (column: Welch Xtimate C18 40*200 mm 7 um; mobile phase: water (NH₄HCO)-ACN; gradient: 26%-66% B over 14 min). Product trans-4-((4-(2-(2-aminopyridin-3-yl)-5-phenyl-3H-imidazo[4,5-b]pyridin-3-yl)benzyl)(methyl)amino)cyclohexane-1-carboxamide 1-187 (188 mg, 0.351 mmol, 67% yield) was obtained as a light yellow solid. ¹HNMR (400 MHz, DMSO-d₆) δ_{H}=8.27 (d, J=8.4 Hz, 1H), 8.05-7.96 (m, 4H), 7.50-7.37 (m, 7H), 7.21-7.12 (m, 2H), 7.04 (s, 2H), 6.66 (s, 1H), 6.39 (dd, J=4.8, 7.6 Hz, 1H), 3.64 (s, 2H), 2.41 (d, J=7.2 Hz, 1H), 2.15 (s, 3H), 2.07-1.97 (m, 1H), 1.89-1.78 (m, 4H), 1.40-1.26 (m, 4H). HPLC Rₜ=3.723 min in 8 min chromatography, purity 99.6%. LCMS Rₜ=1.348 min in 4 min chromatography, purity 99.3%, MS ESI calcd. For 531.27, [M+H]⁺ 532.27, found 532.3.

### Example 179 and 180. Synthesis of (1r,4r)-4-((4-(2-(2-aminopyridin-3-yl)-5-phenyl-3H-imidazo[4,5-b]pyridin-3-yl)benzyl)(2,2,2-trifluoroethyl)amino)cyclohexane-1-carboxylic acid (Compound I-188) and (1s,4s)-4-((4-(2-(2-aminopyridin-3-yl)-5-phenyl-3H-imidazo[4,5-b]pyridin-3-yl)benzyl)(2,2,2-trifluoroethyl)amino)cyclohexane-1-carboxylic acid (Compound I-189)

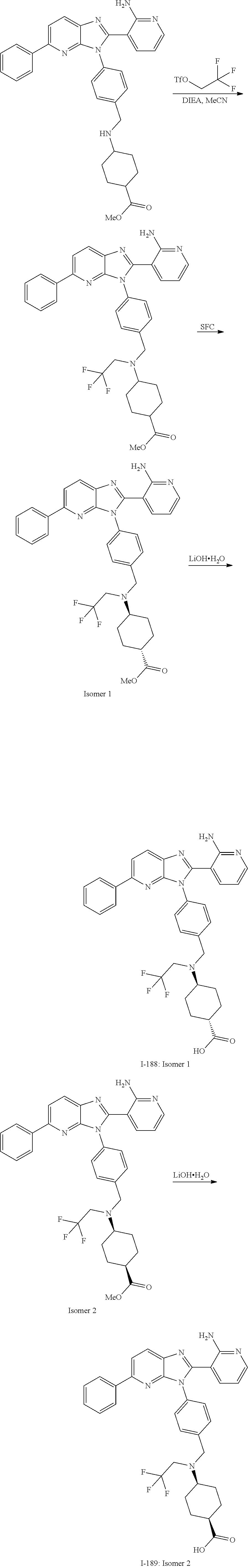

Step 1. To a solution of methyl 4-((4-(2-(2-aminopyridin-3-yl)-5-phenyl-3H-imidazo[4,5-b]pyridin-3-yl)benzyl)amino)cyclohexane-1-carboxylate (1320 mg, 0.995 mmol) in MeCN (15 mL) was added 2,2,2-trifluoroethyl trifluoromethanesulfonate (865 mg, 1.49 mmol) and DIEA (1285 mg, 3.98 mmol) at 25° C. The mixture was stirred at 60° C. for 4 h. The residue was purified by flash silica gel chromatography (eluent of 0-5% MeOH in DCM). The residue was purified by prep-HPLC (Column: F-Prepulite XP tC 18 40*200 mm*7 um; mobile phase: [water (FA)-ACN]; B %: 34-74%; 20.5 min). Compound methyl 4-((4-(2-(2-aminopyridin-3-yl)-5-phenyl-3H-imidazo[4,5-b]pyridin-3-yl)benzyl)(2,2,2-trifluoroethyl)amino)cyclohexane-1-carboxylate (180 mg, 0.136 mmol, 12% yield) was obtained as light yellow oil. LCMS Rₜ=5.860 min in 7 min chromatography, purity 84%, MS ESI calcd. for 614.67, [M+H]⁺ 615.67, found 615.3.

Step 2. The product methyl 4-((4-(2-(2-aminopyridin-3-yl)-5-phenyl-3H-imidazo[4,5-b]pyridin-3-yl) benzyl)(2,2,2-trifluoroethyl)amino)cyclohexane-1-carboxylate was purified by SFC (Column: DAICEL CHIRALPAK AD (250 mm*30 mm, 10 um); mobile phase:[CO₂-EtOH (0.1% NH₃H₂O)]; B %: 35%-35%, min). Isomer 1 methyl trans-4-((4-(2-(2-aminopyridin-3-yl)-5-phenyl-3H-imidazo[4,5-b]pyridin-3-yl)benzyl)(2,2,2-trifluoroethyl)amino)cyclohexane-1-carboxylate (90.0 mg) was obtained as light yellow oil. ¹HNMR (400 MHz, DMSO-d₆) δ=8.27 (d, J=8.4 Hz, 1H), 8.07-7.92 (m, 4H), 7.55-7.49 (m, 2H), 7.47-7.35 (m, 5H), 7.19-7.10 (m, 3H), 6.31 (dd, J=4.8, 7.6 Hz, 1H), 3.90 (s, 2H), 3.58 (s, 3H), 2.47-2.39 (m, 2H), 2.34-2.24 (m, IH), 1.95 (d, J=11.2 Hz, 2H), 1.81 (d, J=10.8 Hz, 2H), 1.45-1.17 (m, 5H). Isomer 2 methyl Cis-4-((4-(2-(2-aminopyridin-3-yl)-5-phenyl-3H-imidazo[45-b]pyridin-3-yl)benzyl)(2,2,2-trifluoroethyl)amino)cyclohexane-1-carboxylate (60.0 mg) was obtained as light yellow oil. ¹HNMR (400 MHz, DMSO-d₆) δ=8.26 (d, J=8.4 Hz, 1H), 8.05-7.96 (m, 4H), 7.53-7.48 (m, 2H), 7.46-7.38 (m, 5H), 7.18-7.10 (m, 3H), 6.31 (dd, J=4.8, 7.6 Hz, 1H), 3.87 (s, 2H), 3 63 (s, 3H), 2.62 (s, 2H), 2.12-2.06 (m, 2H), 1.80-1.72 (m, 1H), 1.72-1.65 (m, 2H), 1.44-1.33 (m, 5H).

Step 3. To a solution of methyl 4-((4-(2-(2-aminopyridin-3-yl)-5-phenyl-3H-imidazo[4,5-b]pyridin-3-yl)benzyl)(2,2,2-trifluoroethyl)amino)cyclohexane-1-carboxylate (90.0 mg, 0.146 mmol) in THF (2 mL). Methanol (0.5 mL) Water (0.5 mL) was added LiOH·H₂O (31.0 mg, 0.732 mmol) at 25° C. The mixture was stirred at 25° C. for 12 hr. The reaction mixture was concentrated directly and the residue was purified by prep-HPLC (Column: F-Prepulite XP tC 18 40*200 mm*7 um, mobile phase: [water (HCOONH₄)-ACN], B %: 18-58%; 20 min). Isomer 1 trans-4-((4-(2-(2-aminopyridin-3-yl)-5-phenyl-3H-imidazo[4,5-b]pyridin-3-yl)benzyl)(2,2,2-trifluoroethyl)amino)cyclohexane-1-carboxylic acid 1-188 (73 mg, 0.121 mmol, 82.4% yield) as a white solid. ¹HNMR (400 MHz, DMSO-d₆) δ=8.27 (d, J=8.4 Hz, 1H), 8.01 (dd, J=8.0, 12.4 Hz, 4H), 7.54-7.35 (m, 7H), 7.18-7.11 (m, 3H), 6.31 (dd, J=4-8, 7.6 Hz, 1H), 3.90 (s, 2H), 3.40-3.35 (m, 2H), 2.45-2.40 (m, 1H), 2.20-2.10 (m, 1H), 1.94 (d, J=12.0 Hz, 2H), 1.80 (d, J=10.0 Hz, 2H), 1.44-1.31 (m, 2H), 1.29-1.17 (m, 2H). HPLC Rₜ=3.317 min in 8 min chromatography, purity 99%. LCMS Rₜ=1.456 min in 2.5 min chromatography, purity 97.4%, MS ESI calcd. for 600.25, [M+H]⁺ 601.25, found 601.2. Isomer 2 cis-4-((4-(2-(2-aminopyridin-3-yl)-5-phenyl-3H-imidazo[4,5-b]pyridin-3-yl)benzyl)(2,2,2-trifluoroethyl)amino)cyclohexane-1-carboxylic acid I-189 (19.0 mg, 0.0306 mmol, 31.3% yield) as a white solid. ¹HNMR (400 MHz, DMSO-d₆) δ=8.27 (d, J=8.4 Hz, 1H), 8.07-7.95 (m, 4H), 7.54-7.34 (m, 7H), 7.19-7.10 (m, 3H), 6.31 (dd, J=4.8, 7.6 Hz, 1H), 3.87 (s, 2H), 3.26 (s, 2H), 2.48-2.45 (m, 2H), 2.09 (d, J=10.4 Hz, 2H), 1.73-1.62 (m, 2H), 1.45-1 30 (m, 4H). HPLC Rₜ=3.334 min in 8 min chromatography, purity 99%. LCMS Rₜ=1.439 min in 2.5 min chromatography, purity 97.4%, MS ESI calcd. for 600.25, [M+H]⁺ 601.25, found 601.2.

### Synthesis of Additional Intermediates for Table 4 Compounds

### Synthesis of methyl 4-formyl-2,2,6,6-tetramethylcyclohexane-1-carboxylate

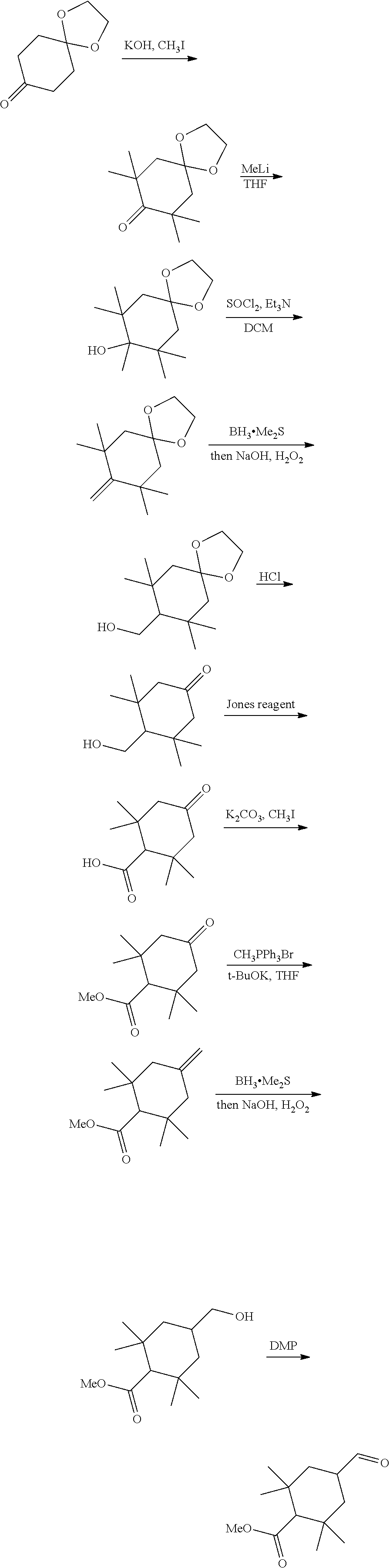

Step 1. To a solution of 1,4-dioxaspiro[4.5]decan-8-one (20 g, 128 mmol) in DMSO (400 mL) was added KOH (143.7 g, 2.56 mol) slowly at 20° C. under N₂. The reaction was heated to 55° C., then CH₃I (145 g, 1.02 mol, 64 mL) was added slowly under N₂. The reaction was stirred at 55° C. under N₂ for 16 hrs. The reaction mixture was poured into ice water (500 mL) and extracted with EtOAc (3×300 mL). The combined organic phase was washed with brine (600 mL*3), dried over Na₂SO₄, filtered and concentrated to give 7,7,9,9-tetramethyl-1,4-dioxaspiro[4.5]decan-8-one (26.0 g, 122 mmol) as yellow oil without purification. ¹HNMR (400 MHz, DMSO-d₆) δ=3.96 (s, 4H), 2.02 (s, 4H), 1.17 (s, 12H).

Step 2. To a solution of 7,7,9,9-tetramethyl-1,4-dioxaspiro[4.5]decan-8-one (28.0 g, 132 mmol) in THF (300 mL) was added MeLi (2 M, 73 mL) at −78° C. slowly under N₂. After stirring at −78° C. under N₂ for 6 hrs, the reaction was warmed up to 20° C. and stirred at 20° C. under N₂ for 16 hrs The reaction mixture cooled to 0° C. and quenched with sat aq. NH₄Cl (500 mL) under N₂. The mixture was extracted with EtOAc (3×300 mL). The combined organic phase was washed with brine (1000 mL), dried over Na₂SO₄, filtered and concentrated. The residue was purified by silica gel flash chromatography (eluent of 0-10% EtOAc in PE) to give 7,7,8,9,9-pentamethyl-1,4-dioxaspiro[4.5]decan-8-ol (20 g, 88 mmol) as colorless oil. ¹HNMR (400 MHz, CHLOROFORM-d) δ_{H}=4.02-3.94 (m, 2H), 3.89-3.82 (m, 2H), 1.90 (d, J=13.6 Hz, 2H), 1.47-1.37 (m, 3H), 1.18 (s, 6H), 1.18 (s, 3H), 1.01 (s, 6H).

Step 3. To a solution of 7,7,8,9,9-pentamethyl-1,4-dioxaspiro[4.5]decan-8-ol (20.0 g, 87.6 mmol) in DCM (300 mL) was added Et₃N (88.6 g, 876 mmol, 122 mL) slowly at 20° C. The reaction was cooled to −78° C. and SOCl₂ (31.26 g, 263 mmol, 19.1 mL) was added at −78° C. under N₂. The reaction was warmed up to 20° C. and stirred under N₂ for 3 hrs. The reaction mixture was poured into NaHCO₃ (300 mL) slowly and extracted with DCM (200 mL*3). The combined organic phase was washed with H₂O (500 mL), dried over Na₂SO₄, filtered and concentrated. The residue was purified by silica gel flash chromatography (eluent of 0~10% EtOAc in PE) to give 7,7,9,9-tetramethyl-8-methylene-1,4-dioxaspiro[4.5]decane (9.0 g, 42 8 mmol) as brown oil. ¹HNMR (400 MHz, CHLOROFORM-d)=4.98 (s, 2H), 3.92 (s, 4H), 1.73 (s, 4H), 1.18 (s, 12H).

Step 4. To a solution of 7,7,9,9-tetramethyl-8-methylene-1,4-dioxaspiro[4.5]decane (9.00 g, 42.8 mmol) in THF (100 mL) was added BH₃-Me₂S (10 M, 8.6 mL) slowly at 0° C. under N₂. After stirring at 20° C. under N₂ for 16 hrs, NaOH (2 M, 107 mL) (2 M) was added at 0° C. Then H₂O₂ (7.07 g, 62 3 mmol, 6 mL, 30/a purity) was added slowly to the reaction at 0° C. under N₂ and the reaction was stirred at 0° C. under N₂ for 1 hr. The layers were separated and extracted with EtOAc (3×100 mL). The combined organic phase was washed with Na₂S₂O₃ (500 mL), brine (500 mL), dried over Na₂SO₄, filtered and concentrated. The residue was purified by silica gel flash chromatography (eluent of 0~20% EtOAc in PE) to give (7,7,9,9-tetramethyl-1,4-dioxaspiro[4.5]decan-8-yl)methanol (6.6 g, 28.9 mmol, 68% yield) as yellow oil. ¹HNMR (400 MHz, CHLOROFORM-d) δ_{H}=4.02-3.95 (m, 2H), 3 87-3.79 (m, 4H), 1.63-1.58 (m, 2H), 1 49-1.45 (m, 2H), 1.19-1.16 (m, 1H), 1.06 (s, 6H), 1.04 (s, 6H).

Step 5. To a solution of (7,7,9,9-tetramethyl-1,4-dioxaspiro[4.5]decan-8-yl)methanol (6.60 g, 28.9 mmol) in acetone (70 mL) was added HCl (3 M, 48.2 mL). The reaction was stirred at 20° C. under N₂ for 16 hrs. The layers were separated and extracted with EtOAc (3×100 mL). The combined organic phase was washed with brine (30 mL), dried over Na₂SO₄, filtered and concentrated to give 4-(hydroxymethyl)-3,3,5,5-tetramethylcyclohexan-1-one (5.50 g, 26.8 mmol) as yellow oil without purification. ¹HNMR (400 MHz, CHLOROFORM-d) δ_{H}=3.85 (d, J=4.0 Hz, 2H), 2.41-2.32 (m, 2H), 2.14-2.10 (m, 2H), 1.65 (t, J=4.0 Hz, 1H), 1.19 (s, 6H), 0.93 (s, 6H).

Step 6. Jones reagent: CrO₃ (8.14 g, 81.4 mmol) was dissolved in H₂SO₄ (2 M, 81.4 mL). To a solution of 4-(hydroxymethyl)-3,3,5,5-tetramethylcyclohexan-1-one (5.00 g, 27.1 mmol) in acetone (60 mL) was added Jones reagent slowly at 0° C. under N₂. The reaction was stirred at 20° C. under N₂ for 16 hrs. The reaction mixture was quenched with 2-propanol (80 mL) and diluted with water (80 mL) and extracted with DCM (3×100 mL). The combined organic phase was dried over Na₂SO₄, filtered and concentrated to give 2,2,6,6-tetramethyl-4-oxocyclohexane-1-carboxylic acid (4 g, 20.18 mmol) as yellow oil without purification. ¹HNMR (400 MHz, CHLOROFORM-d) δ_{H}=2.62-2.50 (m, 3H), 2.19-2.14 (m, 2H), 1.16 (s, 12H).

Step 7. To a solution of 2,2,6,6-tetramethyl-4-oxocyclohexane-1-carboxylic acid (4.00 g, 20.18 mmol) in DMSO (50 mL) was added K₂CO₃ (8.37 g, 60.5 mmol) and Mel (4.30 g, 30.3 mmol, 1.9 mL) at 20° C. under N₂. The reaction was stirred at 20° C. under N₂ for 3 hrs. The reaction mixture was poured into water (50 mL) and extracted with EtOAC (3×50 mL). The combined organic phase was washed with brine (3×100 mL), dried over Na₂SO₄, filtered and concentrated. The residue was purified by silica gel flash chromatography (eluent of 0~5% EtOAc in PE) to give methyl 2,2,6,6-tetramethyl-4-oxocyclohexane-1-carboxylate (2 20 g, 10.4 mmol) as colorless oil. ¹HNMR (400 MHz, CHLOROFORM-d) δ_{H}=3.71 (s, 3H), 2.60-2.48 (m, 3H), 2.16-2.08 (m, 2H), 1.10 (d, J=4.0 Hz, 12H).

Step 8. To a solution of methyl(triphenyl)phosphonium;bromide (6.73 g, 18.8 mmol) in THF (30 mL) was added t-BuOK (1 M, 18.8 mL). After stirring at 50° C. under N₂ for 1 hr, methyl 2,2,6,6-tetramethyl-4-oxocyclohexane-1-carboxylate (2 g, 9.42 mmol) in THF (30 mL) was added. The reaction was stirred at 50° C. under N₂ for 3 hrs The reaction mixture was poured into sat aq NH₄Cl (50 mL) and extracted with EtOAc (3×50 mL). The combined organic phase was washed with brine (100 mL), dried over Na₂SO₄, filtered and concentrated. The residue was purified by silica gel flash chromatography (eluent of 0~5% EtOAc in PE) to give methyl 2,2,6,6-tetramethyl-4-methylenecyclohexane-1-carboxylate (1.50 g, 7.13 mmol) as colorless oil. ¹HNMR (400 MHz, DMSO-d₆) δ=4.72 (s, 2H), 3.58 (s, 3H), 2.25 (s, 1H), 2.04-1 99 (m, 2H), 1 93-1.85 (m, 2H), 0.96 (s, 6H), 0.90 (s, 6H).

Step 9. To a solution of methyl 2,2,6,6-tetramethyl-4-methylenecyclohexane-1-carboxylate (1.4 g, 6.66 mmol) in THF (15 mL) was added BH₃-Me₂S (10 M, 1.66 mL) slowly at 0° C. under N₂. After stirring at 0° C. under N₂ for 0.5 hr, EtOH (20 mL) was added dropwise follow by NaOH (3 M, 11.1 mL) and stirred at 0° C. for 0.5 hr. Then H₂O₂ (3.77 g, 33.3 mmol, 3.2 mL, 30% purity) was added slowly to the reaction at 0° C. under N₂ and the reaction was stirred at 20° C. under N₂ for 0.5 hr. The layers were separated and extracted with EtOAc (3×50 mL). The combined organic phase was washed with Na₂S₂O₃ (150 mL), brine (50 mL), dried over Na₂SO₄, filtered and concentrated. The residue was purified by silica gel flash chromatography (eluent of 0~10% EtOAc in PE) to give methyl 4-(hydroxymethyl)-2,2,6,6-tetramethylcyclohexane-1-carboxylate (1.50 g, 6.57 mmol) as colorless oil. ¹HNMR (400 MHz, DMSO-d₆) δ=4.41 (t, J=5.2 Hz, 1H), 3.52 (s, 3H), 3.22 (t, J=5.6 Hz, 2H), 2.09 (s, 1H), 1.82-1.67 (m, 1H), 1.37-1.27 (m, 2H), 1.26-1.18 (m, 2H), 1.09 (s, 6H), 0.82 (s, 6H).

Step 10. To a solution of methyl 4-(hydroxymethyl)-2,2,6,6-tetramethylcyclohexane-1-carboxylate (250 mg, 1 09 mmol) in DCM (5 mL) was added DMP (929 mg, 2.19 mmol). The reaction was stirred at 20° C. under N₂ for 2 hrs. The reaction mixture was poured into water (10 mL) and extracted with DCM (3×10 mL). The combined organic phase was washed with brine(30 mL), dried over Na₂SO₄, filtered and concentrated to give methyl 4-formyl-2,2,6,6-tetramethylcyclohexane-1-carboxylate (200 mg, 884 μmol) as colorless oil without purification. ¹HNMR (CDCl₃-d₆, 400 MHz) δ_{H}=9.60 (s, 1H), 3.54 (s, 3H), 2.72-2 62 (m, 1H), 2.13 (s, 1H), 1.63-1.54 (m, 2H), 1.47-1.37 (m, 2H), 1.12 (s, 6H), 0.87 (s, 6H).

### Compounds Prepared in Accordance with General Scheme 4

The compounds herein (such as a compound of Formula I or Formula I, or other compounds in Section 1) may be prepared according to General Scheme 4, for example, from an intermediate as prepared in “Synthesis of additional intermediates for Table 4 compounds,” an intermediate that is commercially available, an intermediate as prepared elsewhere in this application or an intermediate that can be synthesized via a method known to those skilled in the art. An exemplary full synthesis is provided in Example 59 (Compound I-68). Compounds prepared in accordance with General Scheme 4 are provided in Table 4 below.

TABLE 4

| | Compound | | | |
|---|---|---|---|---|
| Example | No. | Structure | 1H NMR | LCMS |
| 181 | I-190 | 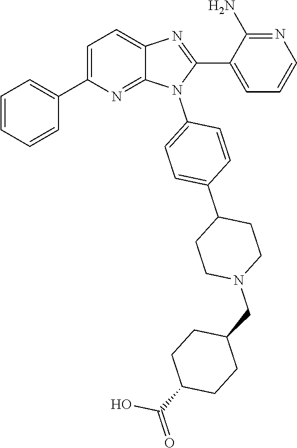 [SMILES: Nc1ncccc1-c1nc2ccc(-c3ccccc3)nc2n1-c1ccc(C2CCN(C[C@H]3CC[C@H](C(=O)O)CC3)CC2)cc1] | ¹H NMR (DMSO, 400 MHz,) δ = 8.26 (d, J = 8.4 Hz, 1H), 8.04-7.95 (m, 4H), 7.49-7.37 (m, 7H), 7.19-7.13 (m, 1H), 6.96 (s, 2H), 6.40 (dd, J = 4.8, 7.2 Hz, 1H), 2.97-2.91 (m, 2H), 2.63-2.54 (m, 1H), 2.16- 2.07 (m, 3H), 2.00-1.79 (m, 8H), 1.74-1.63 (m, 2H), 1.53- 1.43 (m, 1H), 1.35-1.24 (m, 2H), 0.93-0.82 (m, 2H) | MS ESI calcd. for 586.74, [M + H]⁺ 587.74, found 587.3. |
| 182 | I-191 | 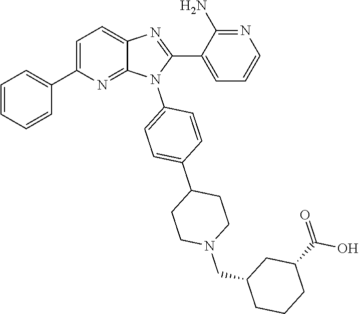 [SMILES: Nc1ncccc1-c1nc2ccc(-c3ccccc3)nc2n1-c1ccc(C2CCN(C[C@H]3CCC[C@@H](C(=O)O)C3)CC2)cc1] | ¹H NMR (400 MHz, DMSO- d₆) δ_{H} = 10.33 (s, 1H), 8.42 (d, J = 8.8 Hz, 1H), 8.19 (d, J] = 8.4 Hz, 1H), 8.08 (dd, J = 1.6, 4.8 Hz, 1H), 7.86 (dd, J] = 1.6, 8.4 Hz, 1H), 7.68 (d, J = 8.8 Hz, 1H), 7.64-7.57 (m, 1H), 7.22-7.10 (m, 3H), 7.01 (s, 2H), 6.45 (dd, J = 4.8, 7.6 Hz, 1H), 2.57-2.50 (m, 1H), 2.47-2.39 (m, 1H), 2.28-2.07 (m, 7H), 1.79-1.55 (m, 4H). | MS ESI calcd. For 586.31, [M + H]⁺ 587.31, found 587.3. |
| 183 | I-192 | 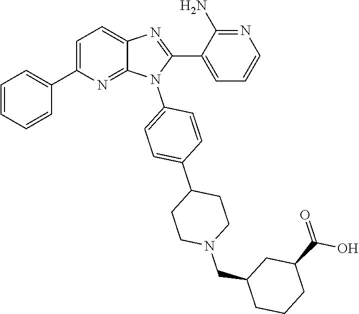 [SMILES: Nc1ncccc1-c1nc2ccc(-c3ccccc3)nc2n1-c1ccc(C2CCN(C[C@@H]3CCC[C@H](C(=O)O)C3)CC2)cc1] | ¹H NMR (400 MHz, DMSO- d₆) δ_{H} = 10.33 (s, 1H), 8.42 (d, J = 8.8 Hz, 1H), 8.19 (d, J = 8.4 Hz, 1H), 8.08 (dd, J = 1.6, 4.8 Hz, 1H), 7.86 (dd, J = 1.6, 8.4 Hz, 1H), 7.68 (d, J = 8.8 Hz, 1H), 7.64-7.57 (m, 1H), 7.22-7.10 (m, 3H), 7.01 (s, 2H), 6.45 (dd, J = 4.8, 7.6 Hz, 1H), 2.57-2.50 (m, 1H), 2.47-2.39 (m, 1H), 2.28-2.07 (m, 7H), 1.79-1.55 (m, 4H). | MS ESI calcd. For 586.31, [M + H]⁺ 587.31, found 587.3. |
| 184 | I-193 | 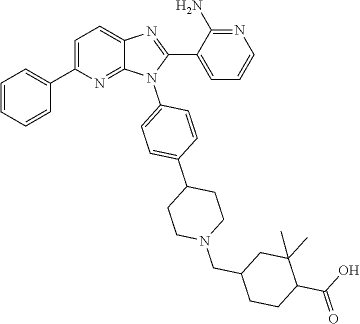 [SMILES: CC1(C)CC(CN2CCC(c3ccc(-n4c(-c5cccnc5N)nc5ccc(-c6ccccc6)nc54)cc3)CC2)CCC1C(=O)O] | ¹H NMR (DMSO-d6, 400 MHz) δ = 8.26 (d, J = 8.4 Hz, 1H), 8.15 (s, 1H), 8.07-7.94 (m, 4H), 7.50-7.37 (m, 7H), 7.16 (dd, J = 1.6, 7.6 Hz, 1H), 6.96 (s, 2H), 6.40 (dd, J = 4.8, 7.6 Hz, 1H), 3.01-2.92 (m, 2H), 2.64-2.57 (m, 1H), 2.20- 2.10 (m, 3H), 2.05-1.95 (m, 2H), 1.85-1.63 (m, 7H), 1.58- 1.41 (m, 2H), 1.30-1.19 (m, 2H), 0.96 (d, J = 14.4 Hz, 6H). | MS ESI calcd. for 614.34 [M + H]⁺ 615.34, found 615.4. |
| 185 | I-194 | 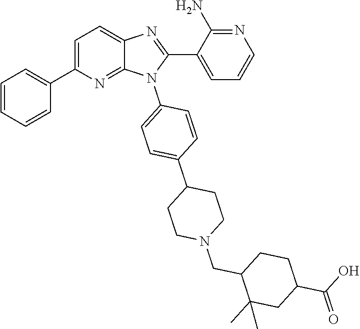 [SMILES: CC1(C)CC(C(=O)O)CCC1CN1CCC(c2ccc(-n3c(-c4cccnc4N)nc4ccc(-c5ccccc5)nc43)cc2)CC1] | ¹H NMR (400 MHz, DMSO- d₆) δ = 8.26 (d, J = 8.4 Hz, 1H), 8.05-7.94 (m, 4H), 7.50- 7.37 (m, 7H), 7.16 (dd, J = 1.6, 7.6 Hz, 1H), 6.97 (s, 2H), 6.39 (dd, J = 4.8, 7.6 Hz, 1H), 3.08-2.99 (m, 1H), 2.96-2.85 (m, 1H), 2.65-2.56 (m, 1H), 2.45-2.35 (m, 1H), 2.34-1.95 (m, 4H), 1.89-1.63 (m, 6H), 1.62-1.18 (m, 5H), 1.02-0.94 (m, 3H), 0.87-0.76 (m, 3H). | MS ESI calcd. for 614.34 [M + H]⁺ 615.34, found 615.4. |
| 186 | I-195 | 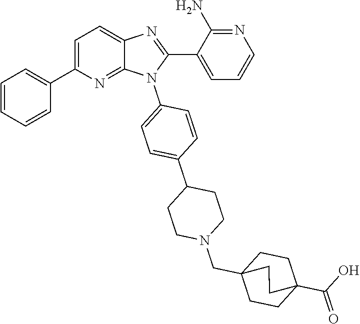 [SMILES: Nc1ncccc1-c1nc2ccc(-c3ccccc3)nc2n1-c1ccc(C2CCN(CC34CCC(C(=O)O)(CC3)CC4)CC2)cc1] | ¹H NMR (400 MHz, DMSO- d₆) δ = 8.27 (d, J = 8.4 Hz, 1H), 8.20 (s, 1H), 8.08-7.93 (m, 4H), 7.53-7.34 (m, 7H), 7.17 (dd, J = 7.6, 2.0 Hz, 1H), 6.98 (s, 2H), 6.40 (dd, J = 7.6, 4.8 Hz, 1H), 2.83 (d, J = 11.2 Hz, 2H), 2.61-2.53 (m, 1H), 2.33-2.23 (m, 2H), 2.05 (s, 2H), 1.83-1.58 (m, 10H), 1.50-1.31 (m, 6H) | MS ESI calcd. for 612.32 [M + H]⁺ 613.32, found 613.4. |
| 187 | I-196 | 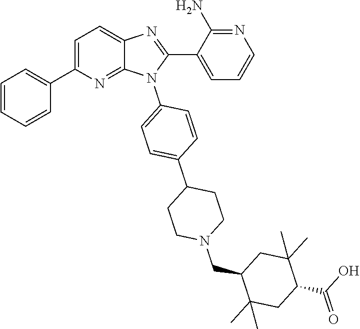 [SMILES: CC1(C)C[C@@H](C(=O)O)C(C)(C)C[C@@H]1CN1CCC(c2ccc(-n3c(-c4cccnc4N)nc4ccc(-c5ccccc5)nc43)cc2)CC1] | ¹H NMR (400 MHz, DMSO- d₆) δ = 8.26 (d, J = 8.4 Hz, 1H), 8.06-7.94 (m, 4H), 7.51- 7.36 (m, 7H), 7.16 (dd, J = 1.6, 7.6 Hz, 1H), 6.97 (s, 2H), 6.44-6.35 (m, 1H), 2.99-2.91 (m, 2H), 2.64-2.55 (m, 1H), 2.09-2.09 (m, 1H), 2.14-2.07 (m, J = 7.0 Hz, 2H), 2.04-1.89 (m, 4H), 1.86-1.77 (m, 2H), 1.76-1.63 (m, 2H), 1.55-1.47 (m, 2H), 1.10 (s, 6H), 0.91 (s, 6H), 0.75 (t, J = 12.8 Hz, 2H). | MS ES calcd. for 642.37 [M + H]⁺ 643.37, found 643.5. |
| 188 | I-197 | 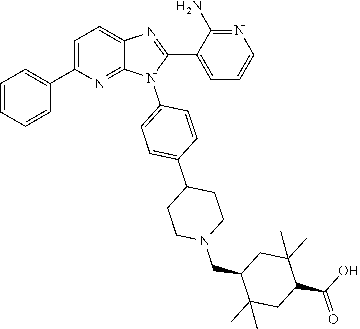 | ¹H NMR (400 MHz, DMSO- d6)δ = 8.28-8.23 (m, 1H), 8.05-7.93 (m, 4H), 7.51-7.36 (m, 7H), 7.16 (dd, J = 1.6, 7.6 Hz, 1H), 6.97 (s, 2H), 6.40 (dd, J = 4.8, 7.6 Hz, 1H), 3.00-2.92 (m, 2H), 2.63-2.55 (m, 1H), 2.18-2.08 (m, 2H), 2.04-1.89 (m, 4H), 1.87-1.79 (m, 2H), 1.77-1.64 (m, 2H), 1.44-1.32 (m, 2H), 1.30-1.20 (m, 2H), 1.10 (s, 6H), 0.92 (s, 6H). | MS ESI calcd. for 642.37 [M + H]⁺ 643.37, found 643.4. |
| 189 | I-198 | 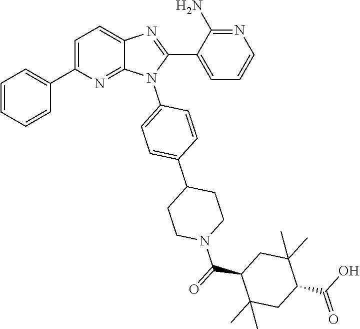 [SMILES: CC1(C)C[C@@H](C(=O)O)C(C)(C)C[C@@H]1C(=O)N1CCC(c2ccc(-n3c(-c4cccnc4N)nc4ccc(-c5ccccc5)nc43)cc2)CC1] | ¹H NMR (DMSO-d₆, 400 MHz) δ_{H} = 8.26 (d, J = 8.4 Hz, 1H), 8.06-7.94 (m, 4H), 7.50-7.37 (m, 7H), 7.17 (dd, J = 1.6, 7.6 Hz, 1H), 6.95 (s, 2H), 6.40 (dd, J = 4.8, 7.6 Hz, 1H), 4.63-4.51 (m, 1H), 4.13-4.02 (m, 1H), 3.18-3.07 (m, 2H), 2.93-2.84 (m, 1H), 2.69-2.55 (m, 2H), 2.05-1.84 (m, 4H), 1.61-1.45 (m, 8H). | MS ESI calcd. for 600.28, [M + H]⁺ 601.28, found 601.3. |
| 190 | I-199 | 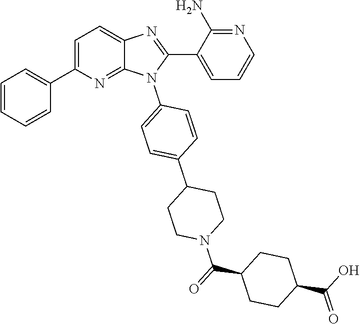 [SMILES: Nc1ncccc1-c1nc2ccc(-c3ccccc3)nc2n1-c1ccc(C2CCN(C(=O)[C@H]3CC[C@@H](C(=O)O)CC3)CC2)cc1] | ¹H NMR (DMSO-d₆, 400 MHz) δ = 8.26 (d, J = 8.4 Hz, 1H), 8.05-7.94 (m, 4H), 7.49- 7.38 (m, 7H), 7.16 (dd, J = 1.6, 7.6 Hz, 1H), 6.95 (s, 2H), 6.40 (dd, J = 4.8, 7.6 Hz, 1H), 4.63-4.52 (m, 1H), 4.16-4.05 (m, 1H), 3.17-3.08 (m, 2H), 2.93-2.85 (m, 1H), 2.65-2.56 (m, 2H), 2.22-2.10 (m, 1H), 1.95-1.84 (m, 4H), 1.78-1.67 (m, 2H), 1.64-1.53 (m, 1H), 1.46-1.37 (m, 4H). | MS ESI calcd. for 600.28, [M + H]⁺ 601.28, found 601.3. |

### Compounds Prepared in Accordance with General Scheme 6

The compounds herein (such as a compound of Formula I or Formula IL, or other compounds in Section I) may be prepared according to General Scheme 6, for example, from an intermediate that is commercially available, an intermediate as prepared elsewhere in this application or an intermediate that can be synthesized via a method known to those skilled in the art. Exemplary full syntheses are provided in Examples 40 and 48 (Compounds 1-40 and 1-57). Compounds prepared in accordance with General Scheme 6 are provided in Tables 5 and 6 below.

TABLE 5
Compounds prepared in accordance with General Scheme 6: exemplified by Compound I-57

| | Compound | | | |
|---|---|---|---|---|
| Example | No. | Structure | 1H NMR | LCMS |
| 191 | I-200 | 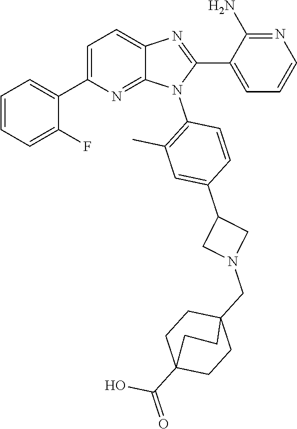 [SMILES: Cc1cc(C2CN(CC34CCC(C(=O)O)(CC3)CC4)C2)ccc1-n1c(-c2cccnc2N)nc2ccc(-c3ccccc3F)nc21] | ¹H NMR (400 MHz,DMSO- d₆) δ = 8.30 (dd, J = 8.4, 1.2 Hz, 1H), 8.01-7.96 (m, 1H), 7.80-7.66 (m, 2H), 7.48-7.41 (m, 1H), 7.39 (s, 1H), 7.36- 7.23 (m, 6H), 7.04 (d, J = 7.6 Hz, 1H), 6.38-6.32 (m, 1H), 3.65 (s, 3H), 3.13 (s, 2H), 2.17 (s, 2H), 1.97 (s, 3H), 1.68-1.58 (m, 6H), 1.40-1.30 (m, 6H). | MS ESI calcd. for 616.30, [M + H]⁺ 617.30, found 617.2. |
| 192 | I-201 | 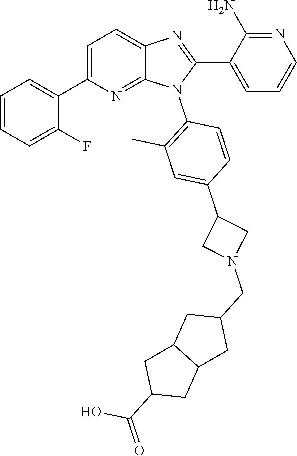 [SMILES: Cc1cc(C2CN(CC3CC4CC(C(=O)O)CC4C3)C2)ccc1-n1c(-c2cccnc2N)nc2ccc(-c3ccccc3F)nc21] | ¹H NMR (METHANOL-d₄ 400 MHz) δ_{H}= 8.53 (s, 1H), 8.23 (d, J = 8.4 Hz, 1H), 7.96 (dd, J = 1.7, 4.8 Hz, 1H), 7.85 (dd, J = 2.0, 8.4 Hz, 1H), 7.80-7.72 (m, 1H), 7.52- 7.45 (m, 1H), 7.44-7.34 (m, 3H), 7.27-7.15 (m, 3H), 6.43- 6.36 (m, 1H), 4.49-4.33 (m, 2H), 4.25-4.09 (m, 3H), 3.23- 3.06 (m, 2H), 2.79-2.44 (m, 3H), 2.07 (s, 7H), 1.83-1.55 (m, 3H), 1.39-0.86 (m, 2H). ¹F NMR (DMSO-d₆ 376.5 MHz) δ_{F} = −118.717. | ESI calcd. for 616.3 [M + H]⁺ 617.3, found 617.2 |
| 193 | I-202 | 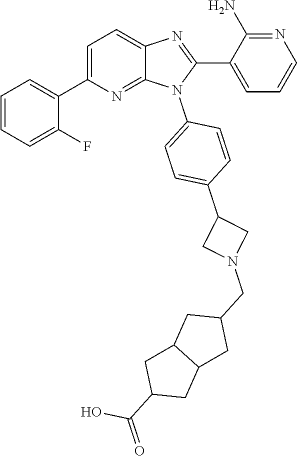 [SMILES: Nc1ncccc1-c1nc2ccc(-c3ccccc3F)nc2n1-c1ccc(C2CN(CC3CC4CC(C(=O)O)CC4C3)C2)cc1] | ¹H NMR (METHANOL-d₄ 400 MHz) δ_{H}= 8.48 (s, 1H), 8.22 (d, J] = 8.4 Hz, 1H), 7.99 (dd, J = 1.6, 4.8 Hz, 1H), 7.91-7.83 (m, 2H), 7.61-7.50 (m, 4H), 7.45-7.38 (m, 1H), 7.36 (dd, J = 2.0, 7.6 Hz, 1H), 7.26-7.17 (m, 2H), 6.51- 6.45 (m, 1H), 4.56-4.43 (m, 2H), 4.31-4.18 (m, 3H), 3.28- 3.16 (m, 2H), 2.87-2.70 (m, 1H), 2.67-2.48 (m, 2H), 2.46- 1.87 (m, 4H), 1.86-1.54 (m, 3H), 1.41-0.87 (m, 2H). ¹F NMR (DMSO-d₆ 376.5 MHz) δ_{F} = −118.640. | MS ESI calcd. for 602.3 [M + H]⁺ 603.3, found 603.2. |
| 194 | I-203 | 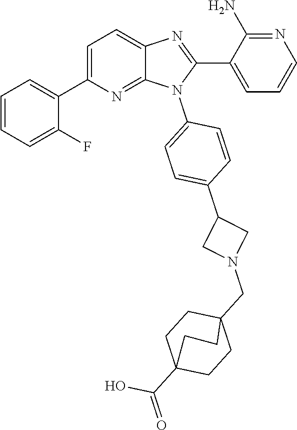 [SMILES: Nc1ncccc1-c1nc2ccc(-c3ccccc3F)nc2n1-c1ccc(C2CN(CC34CCC(C(=O)O)(CC3)CC4)C2)cc1] | ¹H NMR (400 MHz, DMSO- d₆) δ = 8.28 (d, J = 8.4 Hz, 1H), 8.02-7.96 (m, 1H), 7.84- 7.74 (m, 2H), 7.52-7.39 (m, 5H), 7.36-7.28 (m, 2H), 7.20 (dd, J = 7.6, 1.2 Hz, 1H), 6.92 (s, 2H), 6.42 (dd, J = 7.6, 4.8 Hz, 1H), 3.67-3.63 (m, 3H), 3.13 (s, 2H), 2.17 (s, 2H), 1.68-1.56 (m, 6H), 1.40-1.29 (m, 6H). | MS ESI calcd. for 602.28, [M + H]⁺ 603.28, found 603.2. |
| 195 | I-204 | 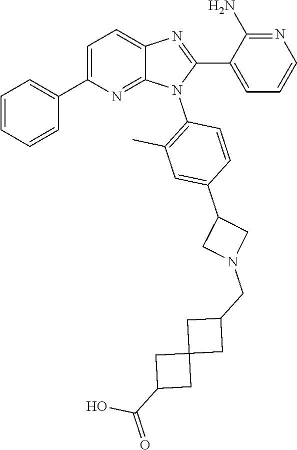 [SMILES: Cc1cc(C2CN(CC3CC4(C3)CC(C(=O)O)C4)C2)ccc1-n1c(-c2cccnc2N)nc2ccc(-c3ccccc3)nc21] | ¹H NMR (400 MHz, DMSO- d₆) δ = 8.28 (d, J = 8.4 Hz, 1H), 8.00-7.93 (m, 4H), 7.50- 7.38 (m, 4H), 7.35-7.25 (m, 4H), 7.03 (d, J = 7.6 Hz, 1H), 6.39-6.29 (m, 1H), 3.68-3.58 (m, 3H), 3.14-3.04 (m, 3H), 2.87 (t, J = 8.4 Hz, 1H), 2.42 (d, J = 6.4 Hz, 2H), 2.19 (d, J = 8.4 Hz, 1H), 2.14 (d, J = 7.6 Hz, 3H), 2.05 (d, J = 8.8 Hz, 2H), 1.97 (s, 3H), 1.74- 1.57 (m, 2H) | MS ESI calcd. for 584.29 [M + H]⁺ 585.29 |
| 196 | I-205 | 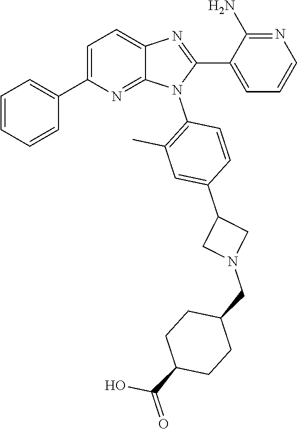 [SMILES: Cc1cc(C2CN(C[C@H]3CC[C@@H](C(=O)O)CC3)C2)ccc1-n1c(-c2cccnc2N)nc2ccc(-c3ccccc3)nc21] | ¹H NMR (400 MHz, DMSO- d₆) δ = 8.29 (d, J = 8.4 Hz, 1H), 8.03-7.93 (m, 4H), 7.53- 7.26 (m, 8H), 7.05 (dd, J = 1.8, 7.8 Hz, 1H), 6.36 (dd, J = 4.8, 7.6 Hz, 1H), 3.78- 3.56 (m, 3H), 3.14-3.06 (m, 2H), 2.44-2.40 (m, 1H), 2.33 (d, J = 6.8 Hz, 2H), 1.98 (s, 3H), 1.89-1.75 (m, 2H), 1.63- 1.36 (m, 5H), 1.29-1.14 (m, 2H) | MS ESI calcd. for 572.29, [M + H]⁺ 573.29, found 573.4 |
| 197 | I-206 | 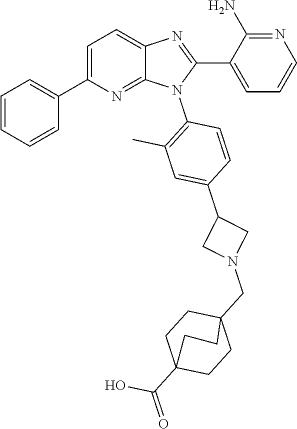 [SMILES: Cc1cc(C2CN(CC34CCC(C(=O)O)(CC3)CC4)C2)ccc1-n1c(-c2cccnc2N)nc2ccc(-c3ccccc3)nc21] | ¹H NMR (400 MHz, DMSO- d₆) δ = 8.28 (d, J = 8.4 Hz, 1H), 8.01-7.93 (m, 4H), 7.49- 7.25 (m, 8H), 7.03 (dd, J = 7.6, 1.6 Hz, 1H), 6.35 (dd, J = 7.6, 4.8 Hz, 1H), 3.70- 3.64 (m, 3H), 3.14 (d, J = 2.0 Hz, 2H), 2.17 (s, 2H), 1.97 (s, 3H), 1.64-1.53 (m, 6H), 1.37-1.28 (m, 6H). | MS ESI calcd. for 598.31, [M + H]⁺ 599.31, found 599.3. |
| 198 | I-207 | 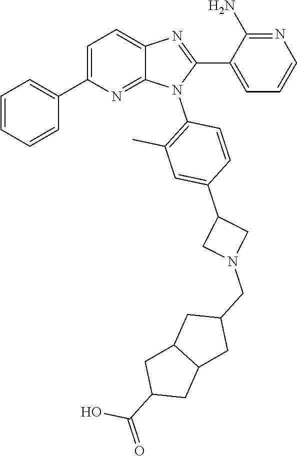 [SMILES: Cc1cc(C2CN(CC3CC4CC(C(=O)O)CC4C3)C2)ccc1-n1c(-c2cccnc2N)nc2ccc(-c3ccccc3)nc21] | ¹H NMR (METHANOL-d₄ 400 MHz) δ_{H} = 8.22 (d, J = 8.4 Hz, 1H), 7.98-7.91 (m, 4H), 7.53-7.48 (m, 1H), 7.44- 7.34 (m, 5H), 7.23 (dd, J = 1.2, 7.2 Hz, 1H), 6.43-6.36 (m, 1H), 4.53-4.41 (m, 2H), 4.31-4.18 (m, 3H), 3.28-3.15 (m, 2H), 2.86-2.70 (m, 1H), 2.68-2.46 (m, 2H), 2.08 (s, 7H), 1.85-1.56 (m, 3H), 1.43- 1.31 (m, 1H), 1.18-0.87 (m, 1H). | MS ESI calcd. for 598.3, [M + H]⁺ 599.3, found 599.1. |
| 199 | I-208 | 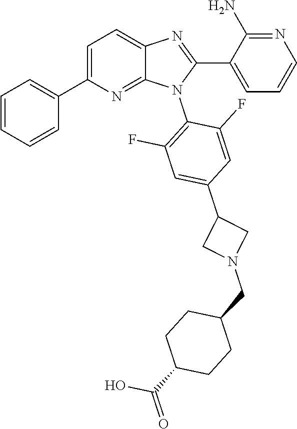 [SMILES: Nc1ncccc1-c1nc2ccc(-c3ccccc3)nc2n1-c1c(F)cc(C2CN(C[C@H]3CC[C@H](C(=O)O)CC3)C2)cc1F] | ¹H NMR (DMSO-d₆, 400 MHz) δ = 8.34 (d, J = 8.4 Hz, 1H), 8.09-7.96 (m, 4H), 7.52- 7.38 (m, 5H), 7.26-7.14 (m, 3H), 6.48 (dd, J = 7.6, 4.8 Hz, 1H), 3.78-3.68 (m, 1H), 3.59 (t, J = 7.2 Hz, 2H), 3.16 (t, J = 6.4 Hz, 2H), 2.29 (d, J = 6.8 Hz, 2H), 2.15-2.07 (m, 1H), 1.88 (d, J = 10.8 Hz, 2H), 1.79 (d, J = 11.2 Hz, 2H), 1.33-1.19 (m, 3.2H), 0.97-0.83 (m, 2H). | MS ESI calcd for 594.26 [M + H]⁺ 595.26, found 595.3. |
| 200 | I-209 | 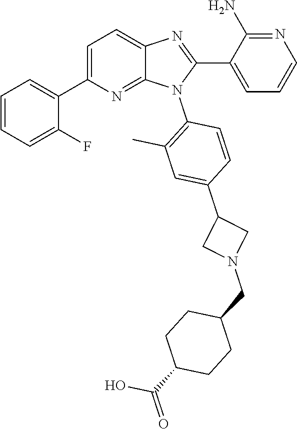 [SMILES: Cc1cc(C2CN(C[C@H]3CC[C@H](C(=O)O)CC3)C2)ccc1-n1c(-c2cccnc2N)nc2ccc(-c3ccccc3F)nc21] | ¹H NMR (400 MHz, DMSO- d₆) δ_{H} = 8.31 (d, J = 8.4 Hz, 1H), 8.18 (s, 1H), 7.99 (dd, J = 1.6, 4.8 Hz, 1H), 7.81- 7.67 (m, 2H), 7.49-7.39 (m, 2H), 7.37-7.22 (m, 6H), 7.05 (dd, J = 1.6. 7.6 Hz, 1H), 6.36 (dd, J = 4.8, 7.6 Hz, 1H), 3.67-3.63 (m, 3H), 3.13 (s, 2H), 2.33-2.28 (m, 2H), 2.16-2.04 (m, 1H), 1.97 (s, 3H), 1.93-1.83 (m, 2H), 1.79 (d, J = 10.4 Hz, 2H), 1.34- 1.17 (m, 3H), 0.98-0.83 (m, 2H) | MS ESI calcd. For 590.28, [M + H]⁺ 591.28, found 591.3. |
| 201 | I-210 | 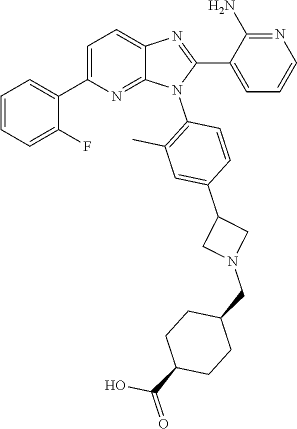 [SMILES: Cc1cc(C2CN(C[C@H]3CC[C@@H](C(=O)O)CC3)C2)ccc1-n1c(-c2cccnc2N)nc2ccc(-c3ccccc3F)nc21] | ¹H NMR (400 MHz, DMSO- d₆) δ_{H} = 8.31 (d, J = 8.4 Hz, 1H), 7.99 (dd, J] = 1.6, 4.8 Hz, 1H), 7.76 (dd, J = 2.0, 8.4 Hz, 1H), 7.74-7.68 (m, 1H), 7.48- 7.39 (m, 2H), 7.35-7.25 (m, 6H), 7.04 (dd, J = 1.6, 7.6 Hz, 1H), 6.35 (dd, J = 4.8, 7.6 Hz, 1H), 3.65-3.59 (m, 3H), 3.10- 3.05 (m, 2H), 2.46-2.38 (m, 1H), 2.31 (d, J = 6.8 Hz, 2H), 1.97 (s, 3H), 1.80 (dd, J = 4.0, 9.2 Hz, 2H), 1.58-1.36 (m, 5H), 1.27-1.12 (m, 2H) | MS ESI calcd. For 590.28, [M + H]⁺ 591.28, found 591.3. |
| 202 | I-211 | 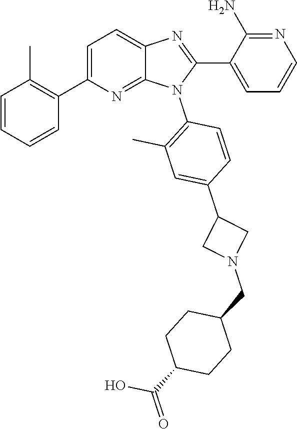 [SMILES: Cc1ccccc1-c1ccc2nc(-c3cccnc3N)n(-c3ccc(C4CN(C[C@H]5CC[C@H](C(=O)O)CC5)C4)cc3C)c2n1] | ¹H NMR (400 MHz, DMSO- d₆) δ = 8.28 (d, J = 8.4 Hz, 1H), 7.98 (dd, J = 4.8, 1.6 Hz, 1H), 7.50 (d, J = 8.0 Hz, 1H), 7.39-7.23 (m, 9H), 7.08 (dd, J = 8.0, 2.0 Hz, 1H), 6.36 (dd, J = 7.6, 4.8 Hz, 1H), 3.65- 3.57 (m, 3H), 3.10-3.03 (m, 2H), 2.29-2.22 (m, 5H), 2.14- 2.03 (m, 1H), 1.93 (s, 3H), 1.86 (d, J = 12.0 Hz, 2H), 1.78 (d, J = 10.4 Hz, 2H), 1.32-1.19 (m, 3H), 0.96-0.82 (m, 2H). | MS ESI calcd. for 586.31, [M + H]⁺ 587.31, found 587.2. |
| 203 | I-212 | 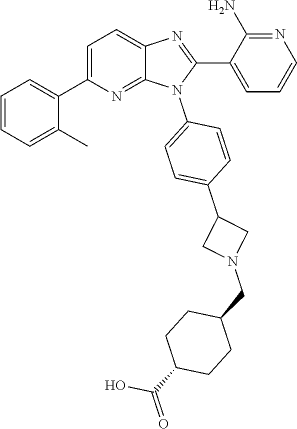 [SMILES: Cc1ccccc1-c1ccc2nc(-c3cccnc3N)n(-c3ccc(C4CN(C[C@H]5CC[C@H](C(=O)O)CC5)C4)cc3)c2n1] | ¹H NMR (400 MHz, DMSO- d₆) δ = 8.26 (d, J = 8.0 Hz, 1H), 8.23 (s, 1H), 7.99 (dd, J = 1.6, 4.8 Hz, 1H), 7.53- 7.44 (m, 3H), 7.42-7.35 (m, 3H), 7.32-7.24 (m, 3H), 7.24- 7.20 (m, 1H), 6.96 (s, 2H), 6.42 (dd, J = 4.8, 7.6 Hz, 1H), 3.65 (d, J] = 3.2 Hz, 3H), 3.17-3.11 (m, 2H), 2.35-2.28 (m, 5H), 2.13-2.05 (m, 1H), 1.89-1.70 (m, 4H), 1.31-1.19 (m, 3H), 0.94-0.83 (m, 2H). | MS ESI calcd. for 572.29 [M + H]⁺ 573.29, found 573.5. |
| 204 | I-213 | 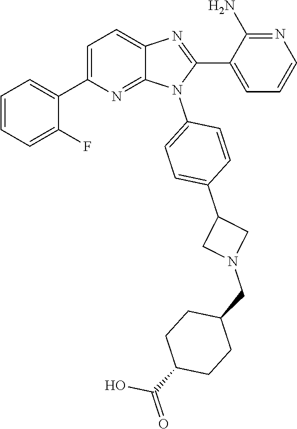 [SMILES: Nc1ncccc1-c1nc2ccc(-c3ccccc3F)nc2n1-c1ccc(C2CN(C[C@H]3CC[C@H](C(=O)O)CC3)C2)cc1] | ¹HNMR (400 MHz, DMSO- d₆) δ = 8.29 (d, J = 8.4 Hz, 1H), 8.18 (s, 1H), 8.00 (dd, J = 1.6, 4.8 Hz, 1H), 7.84- 7.73 (m, 2H), 7.53-7.42 (m, 4H), 7.37-7.26 (m, 2H), 7.21 (dd, J = 2.0, 7.6 Hz, 1H), 6.92 (s, 2H), 6.42 (dd, J = 4.8, 7.6 Hz, 1H), 3.68-3.60 (m, 3H), 3.15-3.09 (m, 2H), 2.30 (d, J = 6.8 Hz, 2H), 2.16-2.02 (m, 1H), 1.93-1.69 (m, 4H), 1.37-1.18 (dq, J = 3.2, 12.8 Hz, 3H), 0.98-0.81 (dq, J = 3.2, 12.8 Hz, 2H). ¹⁹F NMR (376.5 MHz, DMSO-d₆) δ = −117.220 | MS ESI calcd. For 576.26 [M + H]⁺ 577.26, found 577.2. |

TABLE 6
Compounds prepared in accordance with General Scheme 6: exemplified by Compound I-40

| | Compound | | | |
|---|---|---|---|---|
| Example | No | Structure | 1H NMR | LCMS |
| 205 | I-214 | 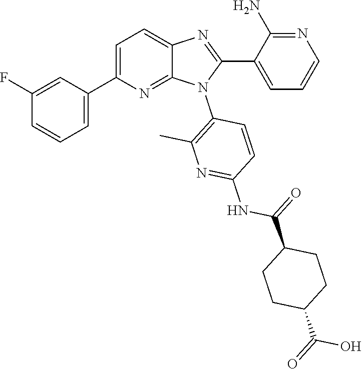 [SMILES: Cc1nc(NC(=O)[C@H]2CC[C@H](C(=O)O)CC2)ccc1-n1c(-c2cccnc2N)nc2ccc(-c3cccc(F)c3)nc21] | ¹HNMR (DMSO-d₆, 400 MHz,) δ = 10.68 (s, 1H), 8.35-8.29 (m, 1H), 8.12 (d, J = 8.8 Hz, 1H), 8.07 (d, J = 8.4 Hz, 1H), 8.03 (dd, J = 1.6, 4.8 Hz, 1H), 7.87 (d, J = 8.4 Hz, 2H), 7.82-7.76 (m, 1H), 7.55- 7.47 (m, 1H), 7.26-7.13 (m, 4H), 6.47 (dd, J = 5.2, 7.6 Hz, 1H), 2.57- 2.51 (m, 1H), 2.20 (tt, J = 3.2, 12.0 Hz, 1H), 2.06 (s, 3H), 1.99-1.87 (m, 4H), 1.53-1.40 (m, 2H), 1.39-1.27 (m, 2H). | MS ESI calcd. for 565.22 [M + H]⁺ 566.2, found 566.2. |
| 206 | I-215 | 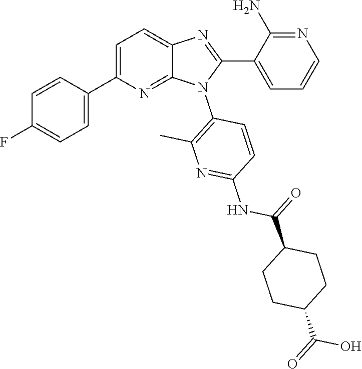 [SMILES: Cc1nc(NC(=O)[C@H]2CC[C@H](C(=O)O)CC2)ccc1-n1c(-c2cccnc2N)nc2ccc(-c3ccc(F)cc3)nc21] | ¹HNMR (DMSO-d₆, 400 MHz,) δ = 10.68 (s, 1H), 8.35-8.29 (m, 1H), 8.12 (d, J = 8.8 Hz, 1H), 8.07 (d, J = 8.4 Hz, 1H), 8.03 (dd, J = 1.6, 4.8 Hz, 1H), 7.87 (d, J = 8.4 Hz, 2H), 7.82-7.76 (m, 1H), 7.55- 7.47 (m, 1H), 7.26-7.13 (m, 4H), 6.47 (dd, J = 5.2, 7.6 Hz, 1H), 2.57- 2.51 (m, 1H), 2.20 (tt, J = 3.2, 12.0 Hz, 1H), 2.06 (s, 3H), 1.99-1.87 (m, 4H), 1.53-1.40 (m, 2H), 1.39-1.27 (m, 2H). | MS ESI calcd. for 565.22 [M + H]+ 566.2, found 566.2. |
| 207 | I-216 | 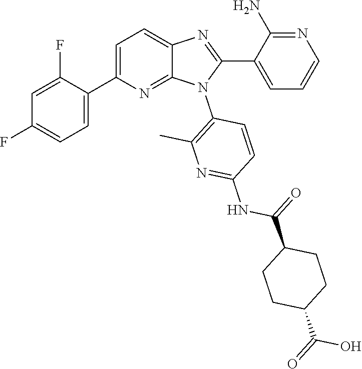 [SMILES: Cc1nc(NC(=O)[C@H]2CC[C@H](C(=O)O)CC2)ccc1-n1c(-c2cccnc2N)nc2ccc(-c3ccc(F)cc3F)nc21] | ¹HNMR (DMSO-d₆, 400 MHz) δ = 10.66 (s, 1H), 8.33 (d, J = 8.4 Hz, 1H), 8.12-8.00 (m, 2H), 7.88- 7.76 (m, 3H), 7.42-7.34 (m, 1H), 7.24-7.12 (m, 4H), 6.47 (dd, J = 4.8, 7.6 Hz, 1H), 2.56-2.51 (m, 1H), 2.24-2.14 (m, 1H), 2.06 (s, 3H), 1.98- 1.86 (m, 4H), 1.51-1.39 (m, 2H), 1.37-1.26 (m, 2H). | MS ESI calcd. for 583.21, [M + H]⁺ 584.21, found 584.2. |
| 208 | I-217 | 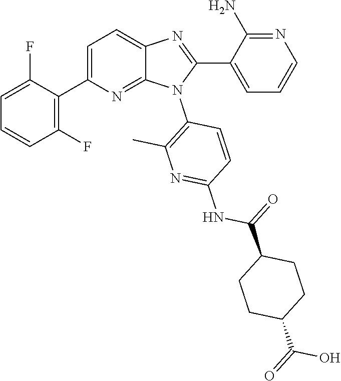 [SMILES: Cc1nc(NC(=O)[C@H]2CC[C@H](C(=O)O)CC2)ccc1-n1c(-c2cccnc2N)nc2ccc(-c3c(F)cccc3F)nc21] | ¹HNMR (400 MHz, MeOD) δ = 8.29 (d, J = 8.4 Hz, 1H), 8.10 (d, J = 8.8 Hz, 1H), 8.01 (dd, J = 2.0, 5.2 Hz, 1H), 7.70 (d, J = 8.8 Hz, 1H), 7.57 (d, J = 8.4 Hz, 1H), 7.44 (tt, J = 6.4, 8.4 Hz, 1H), 7.31 (dd, J = 1.6, 8.0 Hz, 1H), 7.12-7.00 (m, 2H), 6.52 (dd, J = 5.2, 7.6 Hz, 1H), 2.51-2.41 (m, 1H), 2.35-2.25 (m, 1H), 2.15 (s, 3H), 2.08 (d, J = 12.0 Hz, 2H), 2.00 (dt, J = 3.2, 6.8 Hz, 2H), 1.66-1.43 (m, 4H). | MS ESI calcd. for 583.21 [M + H]⁺ 584.21, found 584.2. |
| 209 | I-218 | 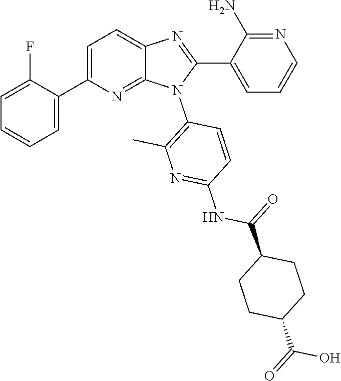 [SMILES: Cc1nc(NC(=O)[C@H]2CC[C@H](C(=O)O)CC2)ccc1-n1c(-c2cccnc2N)nc2ccc(-c3ccccc3F)nc21] | ¹HNMR (400 MHz, DMSO-d₆) δ = 10.67 (s, 1H), 8.34 (d, J = 8.4 Hz, 1H), 8.09 (d, J = 8.4 Hz, 1H), 8.03 (dd, J = 1.6, 4.8 Hz, 1H), 7.89-7.74 (m, 3H), 7.54-7.43 (m, 1H), 7.37-7.28 (m, 2H), 7.21 (dd, J = 1.6, 7.6 Hz, 1H), 7.15 (s, 2H), 6.48 (dd, J = 4.8, 7.6 Hz, 1H), 2.56-2.53(m, 1H), 2.25-2.15 (m, 1H), 2.08 (s, 3H), 1.99-1.85 (m, 4H), 1.52-1.39 (m, 2H), 1.34 (dt, J = 2.8, 12.4 Hz, 2H). | MS ESI calcd. for 565.22, [M + H]⁺ 566.22, found 566.3. |
| 210 | I-219 | 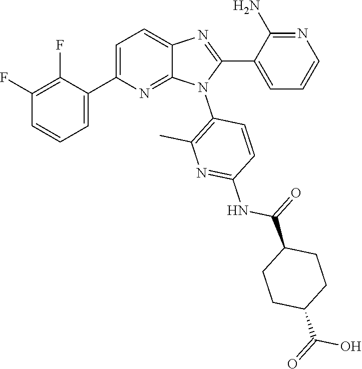 [SMILES: Cc1nc(NC(=O)[C@H]2CC[C@H](C(=O)O)CC2)ccc1-n1c(-c2cccnc2N)nc2ccc(-c3cccc(F)c3F)nc21] | ¹HNMR (400 MHz, DMSO-d₆) δ_{H} = 10.66 (s, 1H), 8.36 (d, J = 8.4 Hz, 1H), 8.09 (d, J = 8.8 Hz, 1H), 8.03 (dd, J = 1.6, 4.8 Hz, 1H), 7.87-7.80 (m, 2H), 7.57 (t, J = 7.2 Hz, 1H), 7.52-7.43 (m, 1H), 7.34-7.26 (m, 1H), 7.21 (dd, J = 1.6, 7.6 Hz, 1H), 7.15 (s, 2H), 6.47 (dd, J = 4.8, 7.6 Hz, 1H), 2.53 (s, 1H), 2.24-2.13 (m, 1H), 2.07 (s, 3H), 1.95 (d. J = 11.2 Hz, 2H), 1.88 (d, J = 10.4 Hz, 2H), 1.51-1.38 (m, 2H), 1.38-1.26 (m, 2H). | MS ESI calcd. For 583.21, [M + H]⁺ 584.21, found 584.3. |
| 211 | I-220 | 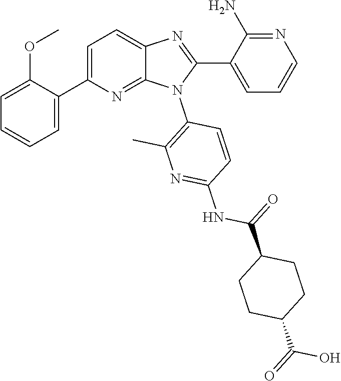 [SMILES: COc1ccccc1-c1ccc2nc(-c3cccnc3N)n(-c3ccc(NC(=O)[C@H]4CC[C@H](C(=O)O)CC4)nc3C)c2n1] | ¹H NMR (400 MHz, DMSO-d₆) δ = 8.20 (d, J = 8.4 Hz, 1H), 8.05- 7.98 (m, 2H), 7.79 (dd, J = 8.4, 14.0 Hz, 2H), 7.50 (dd, J = 1.6, 7.6 Hz, 1H), 7.39-7.32 (m, 1H), 7.18 (dd, J = 1.6, 7.6 Hz, 1H), 7.11 (d, J = 8.4 Hz, 1H), 6.99 (t, J = 7.6 Hz, 1H), 6.45 (dd, J = 4.8, 7.6 Hz, 1H), 3.78 (s, 3H), 2.48-2.41 (m, 1H), 2.19-2.11 (m, 1H), 2.04 (s, 3H), 1.95- 1.81 (m, 4H), 1.46-1.26 (m, 4H) | MS ESI calcd. for 577.24, [M + H]⁺ 578.24, found 578.2. |

### Example 212. Synthesis of (1r,4r)-4-((3-(4-(2-(2-aminopyridin-3-yl)-6-phenyl-1H-benzo[d]imidazol-1-yl)phenyl)azetidin-1-yl)methyl)cyclohexane-1-carboxylic acid (Compound I-221)

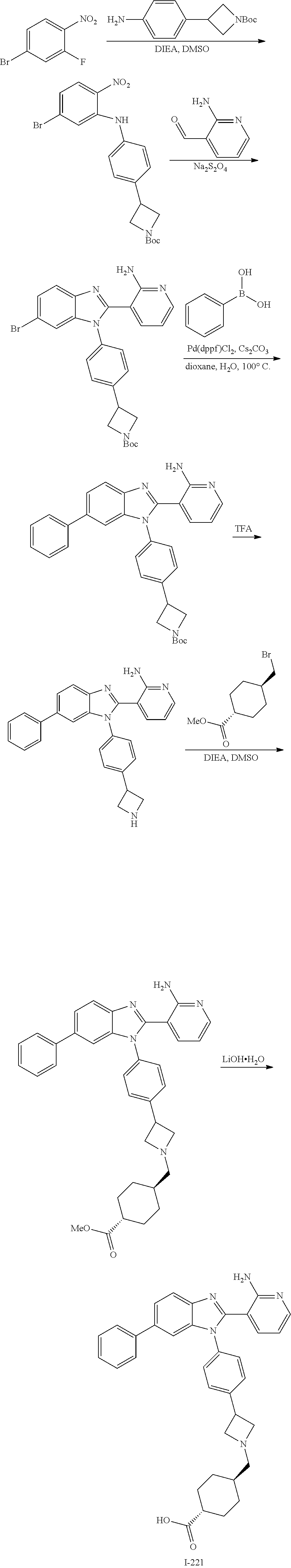

Step 1. To a stirred solution of 4-bromo-2-fluoro-1-nitrobenzene (2.00 g, 9.10 mmol) and tert-butyl 3-(4-aminophenyl)azetidine-1-carboxylate (3.00 g, 12.1 mmol) in DMSO (30.0 mL) was added DIEA (5.00 mL, 27.3 mmol). The reaction mixture was stirred at 95° C. under N₂ for 12 hrs. The reaction mixture was added H₂O (300 mL) and extracted with EtOAc (3×150 mL). The organic layer was dried over Na₂SO₄, filtered and concentrated in vacuum. The crude was purified by flash column (eluent of 0 50% EtOAc in PE) to give tert-butyl 3-(4-((5-bromo-2-nitrophenyl)amino)phenyl)azetidine-1-carboxylate (4.00 g, 98% yield) as a red solid. ¹HNMR (400 MHz, DMSO-d₆) δ=9.48 (s, 1H), 8.05 (d, J=9.2 Hz, 1H), 7.46-7.38 (m, 2H), 7 37-7.29 (m, 2H), 7.17 (d, J=2.0 Hz, 1H), 7.01 (dd, J=2.0, 9.2 Hz, 1H), 4.35-4.20 (m, 2H), 3.92-3.77 (m, 3H), 1.41 (s, 9H).

Step 2. To a stirred solution of 2-aminonicotinaldehyde (2.10 g, 17.8 mmol) and tert-butyl 3-(4-((5-bromo-2-nitrophenyl)amino)phenyl)azetidine-i-carboxylate (4 00 g, 8.90 mmol) in DMSO (70 mL) and methanol (8 mL) was added Na₂S₂O₄ (6.20 g, 35.7 mmol). The reaction mixture was stirred at 95° C. under 02 for 12 hrs. The reaction mixture was diluted with EtOAc (200 mL) and washed with water (100 mL) and brine (2×100 mL), dried over anhydrous sodium sulfate, filtered and the filtrate was concentrated to afford a residue. The crude product was purified by flash chromatography on silica gel (eluent of 0-95% EtOAc in PE) to afford tert-butyl 3-(4-(2-(2-aminopyridin-3-yl)-6-bromo-1H-benzo[d]imidazol-1-yl)phenyl)azetidine-1-carboxylate (2.00 g, 48% yield) as a yellow solid. ¹H NMR (400 MHz, DMSO-d₆) δ=7.97 (dd, J=2.0, 4.8 Hz, 1H), 7.76 (d, J=8.4 Hz, 1H), 7.61-7 39 (m, 5H), 7.30 (d, J=2.0 Hz, 1H), 7.15 (dd, J=1.6, 7.6 Hz, 1H), 6.97 (s, 2H), 6.39 (dd, J=4.8, 7.6 Hz, 1H), 4.36-4.23 (m, 2H), 3.95-3.85 (m, 3H), 1.41 (s, 9H).

Step 3. To a stirred solution of phenylboronic acid (94.0 mg, 0.770 mmol), tert-butyl 3-(4-(2-(2-aminopyridin-3-yl)-6-bromo-1H-benzo[d]imidazol-1-yl)phenyl)azetidine-i-carboxylate (200 mg, 0.380 mmol) and Cs₂CO₃ (501 mg, 1.54 mmol) in 1,4-Dioxane (6 mL) and water (1 mL) was added Pd(dppf)Cl₂·DCM (50.0 mg) under N₂. The reaction mixture was stirred at 100° C. under N₂ for 12 hrs. The reaction mixture was diluted with EtOAc (200 mL) and washed with water (100 mL) and brine (2×100 mL), dried over anhydrous sodium sulfate, filtered and the filtrate was concentrated to afford a residue. The crude product was purified by flash chromatography on silica gel (eluent of 0 5% MeOH in DCM) to afford tert-butyl 3-(4-(2-(2-aminopyridin-3-yl)-6-phenyl-1H-benzo[d]imidazol-1-yl)phenyl)azetidine-1-carboxylate (150 mg, 75% yield) as yellow oil. ¹HNMR (400 MHz, DMSO-d₆) 3=7.98 (dd, J=2.0, 4.8 Hz, 1H), 7.88 (d, J=8.4 Hz, 1H), 7.68-7.60 (m, 3H), 7.59-7.51 (m, 2H), 7.50-7.40 (m, 4H), 7.37-7.30 (m, 2H), 7.13 (dd, J=2.0, 7.6 Hz, 1H), 7.04 (s, 2H), 6.39 (dd, J=4.8, 7.6 Hz, 1H), 4 29 (s, 2H), 3 97-3.83 (m, 3H), 1.41 (s, 9H).

Step 4. To a s stirred solution of tert-butyl 3-(4-(2-(2-aminopyridin-3-yl)-6-phenyl-1H-benzo[d]imidazol-1-yl)phenyl)azetidine-1-carboxylate (150 mg, 0.290 mmol) in DCM (2 mL) was added TFA (1 mL). The reaction mixture was stirred at 25° C. under N₂ for 12 hrs. The reaction mixture was concentrated and diluted with DCM/MeOH=10/1 (50 mL) and washed with saturated aqueous NaHCO₃ (20 mL*2), dried over anhydrous sodium sulfate, filtered and concentrated to afford 3-(1-(4-(azetidin-3-yl)phenyl)-6-phenyl-1H-benzo[d]imidazol-2-yl)pyridin-2-amine (120 mg, 99% yield) as a yellow solid. ¹HNMR (400 MHz, DMSO-d₆) δ=7.98 (dd, J=2.0, 4.8 Hz, 1H), 7.88 (d, J=8.4 Hz, 1H), 7.69-7.51 (m, 6H), 7.49-7.41 (m, 4H), 7.38-7.28 (m, 2H), 7.14 (dd, J=2.0, 8.0 Hz, 1H), 7.03 (s, 2H), 6.39 (dd, J=4.8, 8.0 Hz, 1H), 4.00 (d, J=8.0 Hz, 1H), 3 90 (t, J=8.0 Hz, 2H), 3.75 (t, J=7.6 Hz, 2H).

Step 5. To a stirred solution of 3-(1-(4-(azetidin-3-yl)phenyl)-6-phenyl-1H-benzo[d]imidazol-2-yl)pyridin-2-amine (200 mg, 0479 mmol) and methyl (1r,4r)-4-(bromomethyl)cyclohexane-1-carboxylate (225 mg, 0.960 mmol) in DMSO (5 mL) was added DIEA (0.4 mL, 1.92 mmol). The reaction mixture was stirred at 80° C. under N₂ for 12 hrs. The reaction mixture was diluted with EtOAc (30 mL) and washed with water (10 mL) and brine (10 mL*2), dried over anhydrous sodium sulfate, filtered and concentrated. The crude product was purified by flash chromatography on silica gel (eluent of 0-8% MeOH in DCM) to afford methyl (1r,4r)-4-((3-(4-(2-(2-aminopyridin-3-yl)-6-phenyl-IH-benzo[d]imidazol-1-yl)phenyl)azetidin-1-yl)methyl)cyclohexane-1-carboxylate (100 mg, 37% yield) as yellow oil. LCMS Rₜ=0.837 min in 1.5 min chromatography, purity 100%, MS ESI calcd. for 571.29 [M+H]⁺ 572.29, found 572.3.

Step 6. To a stirred solution of methyl (1r,4r)-4-((3-(4-(2-(2-aminopyridin-3-yl)-6-phenyl-1H-benzo[d]imidazol-1-yl)phenyl)azetidin-1-yl)methyl)cyclohexane-1-carboxylate (100 mg, 0.180 mmol) in THF (1 mL) and water (1 mL) was added LiOH·H₂O (21.0 mg, 0.530 mmol). After stirring at 30° C. under N₂ for 12 hrs, the reaction mixture was concentrated and adjusted to around 5 with IN HCl. The mixture was purified by prep-HPLC (column: welch xtimate C18 150*30 mm*5 μm, method: water (FA)-CAN, begin B: 15, end B 45) to afford (1r,4r)-4-((3-(4-(2-(2-aminopyridin-3-yl)-6-phenyl-IH-benzo[d]imidazol-1-yl)phenyl)azetidin-1-yl)methyl)cyclohexane-1-carboxylic acid I-221 (33.0 mg, 34% yield) as a white solid. ¹HNMR (400 MHz, DMSO-d₆) δ=12.12 (s, 1H), 7.98 (dd, J=2.0, 4.8 Hz, 1H), 7.88 (d, J=8.4 Hz, 1H), 7.70-7.51 (m, 5H), 7.50-7.39 (m, 4H), 7.38-7.29 (m, 2H), 7.14 (dd, J=2.0, 8.0 Hz, 1H), 7.03 (s, 2H), 6.38 (dd, J=4.8, 7.6 Hz, 1H), 3.78 (s, 3H), 3.29-2.97 (m, 2H), 2.45 (d, J=6.4 Hz, 2H), 2.18-2.04 (m, 1H), 1.94-1.71 (m, 4H), 1.40-1 18 (m, 3H), 1.02-0.84 (m, 2H). HPLC Rₜ=2.585 min in 8 min chromatography, purity 99.2%. LCMS Rₜ=1.515 min in 4 min chromatography, purity 99.0%, MS ESI calcd for 557.28 [M+H]⁺ 558.28, found 558.4.

### Compounds Prepared in Accordance with Example 212

The compounds herein (such as a compound of Formula I or Formula II, or other compounds in Section I) may be prepared according to the synthesis provided for Example 212 (Compound I-221), for example, from an intermediate that is commercially available, an intermediate as prepared elsewhere in this application or an intermediate that can be synthesized via a method known to those skilled in the art. Compounds prepared in accordance with Example 212 are provided in Table 7 below.

TABLE 7

| | Compound | | | |
|---|---|---|---|---|
| Example | No. | Structure | 1H NMR | LCMS |
| 213 | I-222 | 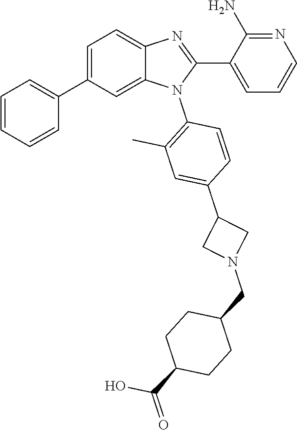 [SMILES: Cc1cc(C2CN(C[C@H]3CC[C@@H](C(=O)O)CC3)C2)ccc1-n1c(-c2cccnc2N)nc2ccc(-c3ccccc3)cc21] | ¹H NMR (400 MHz, DMSO- d₆) δ = 7.96 (dd, J] = 1.6, 4.8 Hz, 1H), 7.89 (d, J = 8.4 Hz, 1H), 7.65-7.57 (m, 3H), 7.48-7.28 (m, 8H), 7.09 (d, J = 1.2 Hz, 1H), 7.05 (dd, J = 1.6, 7.6 Hz, 1H), 6.33 (dd, J = 4.8, 7.6 Hz, 1H), 3.68-3.59 (m, 3H), 3.10 (t, J = 6.0 Hz, 2H), 2.46-2.39 (m, 1H), 2.31 (d, J = 6.8 Hz, 2H), 1.89-1.74 (m, 5H), 1.61-1.35 (m, 5H), 1.28- 1.15 (m, 2H). | MS ESI calcd. For 571.29, [M +H]⁺ 572.29, found 572.3. |
| 214 | I-223 | 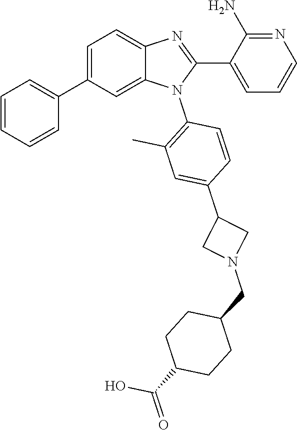 | ¹H NMR (400 MHz, DMSO- d₆) δ = 7.97 (dd, J = 1.6, 4.8 Hz, 1H), 7.89 (d, J = 8.4 Hz, 1H), 7.65-7.57 (m, 3H), 7.46-7.39 (m, 5H), 7.37-7.29 (m, 3H), 7.11-7.03 (m, 2H), 6.33 (dd, J = 4.8, 7.6 Hz, 1H), 3.70-3.59 (m, 3H), 3.11 (t, J = 5.6 Hz, 2H), 2.28 (d, J = 6.8 Hz, 2H), 2.14-2.04 (m, 1H), 1.91-1.76 (m, 7H), 1.26 (dq, J = 3.2, 12.4 Hz, 3H), 0.97-0.84 (m, 2H). | MS ESI calcd. for 571.29 [M + H]⁺ 572.29, found 572.4. |
| 215 | I-224 | 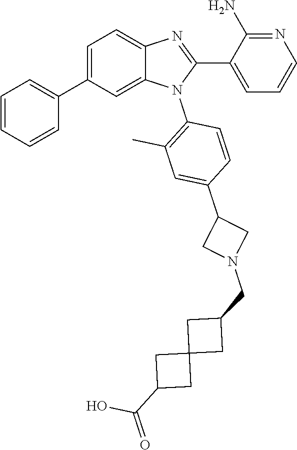 | ¹H NMR (400 MHz, DMSO- d₆) δ = 7.96 (dd, J = 1.6, 4.8 Hz, 1H), 7.89 (d, J = 8.4 Hz, 1H), 7.66-7.56 (m, 3H), 7.38 (d, J = 1.6 Hz, 8H), 7.09 (d, J = 1.2 Hz, 1H), 7.05 (dd, J = 1.6, 8.0 Hz, 1H), 6.33 (dd, J = 4.8, 7.6 Hz, 1H), 3.69- 3.56 (m, 3H), 3.08 (t, J = 6.4 Hz, 2H), 2.58-2.52 (m, 1H), 2.41 (d, J = 6.4 Hz, 2H), 2.14-1.94 (m, 5H), 1.92-1.78 (m, 5H), 1.70-1.58 (m, 1H), 1.53 (dd, J = 7.2, 11.2 Hz, 1H). | MS ESI calcd. for 583.29 [M + H]⁺ 584.29, found 584.3. |

### Compounds Prepared According to a Scheme Similar to Example 128

The compounds herein (such as a compound of Formula I or Formula II, or other compounds in Section I) may be prepared according to the scheme provided for Example 128, for example, from an intermediate that is commercially available, an intermediate as prepared elsewhere in this application or an intermediate that can be synthesized via a method known to those skilled in the art. An exemplary full synthesis is provided in Example 128 (Compound I-137). Compounds prepared in accordance with the scheme provided for Example 128 are provided in Table 8 below.

TABLE 8

| | Compound | | | |
|---|---|---|---|---|
| Example | No. | Structure | 1H NMR | LCMS |
| 216 | I-225 | 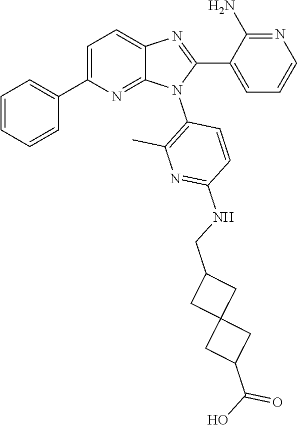 [SMILES: Cc1nc(NCC2CC3(C2)CC(C(=O)O)C3)ccc1-n1c(-c2cccnc2N)nc2ccc(-c3ccccc3)nc21] | ¹H NMR (400 MHz, DMSO-d₆) δ = 8.25 (d, J = 8.4 Hz, 1H), 8.07-7.92 (m, 4H), 7.51-7.33 (m, 4H), 7.26 (dd, J = 2.0, 7.6 Hz, 1H), 7.22 (s, 2H), 6.81 (t, J = 5.6 Hz, 1H), 6.55-6.37 (m, 2H), 3.27-3.21 (m, 2H), 2.90 (t, J = 8.4 Hz, 1H), 2.43- 2.31 (m, 1H), 2.25- 2.08 (m, 5H), 2.05- 1.95 (m, 1H), 1.88 (s, 3H), 1.83-1.65 (m, 2H) | MS ESI calcd. for 545.25 [M + H]⁺ 546.25, found 546.3. |
| 217 | I-226 | 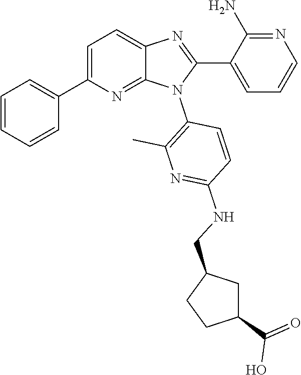 [SMILES: Cc1nc(NC[C@@H]2CC[C@H](C(=O)O)C2)ccc1-n1c(-c2cccnc2N)nc2ccc(-c3ccccc3)nc21] | ¹H NMR (400 MHz, DMSO-d₆) δ = 8.26 (d, J = 8.4 Hz, 1H), 8.04-7.92 (m, 4H), 7.50-7.45 (m, 2H), 7.42-7.36 (m, 2H), 7.29-7.19 (m, 3H), 6.89 (t, J = 5.2 Hz, 1H), 6.50-6.41 (m, 2H), 3.26-3.14 (m, 2H), 2.79-2.64 (m, 1H), 2.25-2.15 (m, 1H), 2.14-2.03 (m, 1H), 1.89 (s, 3H), 1.85-1.73 (m, 3H), 1.49-1.32 (m, 2H) | MS ESI calcd. For 519.24 [M + H]⁺ 520.24, found 520.4. |
| 218 | I-227 | 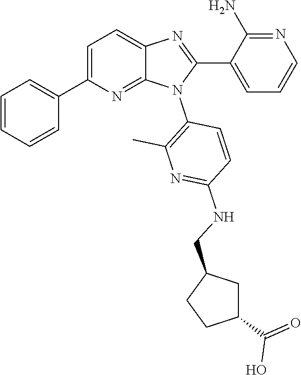 [SMILES: Cc1nc(NC[C@@H]2CC[C@@H](C(=O)O)C2)ccc1-n1c(-c2cccnc2N)nc2ccc(-c3ccccc3)nc21] | ¹H NMR (400 MHz, DMSO-d₆) δ = 8.26 (d, J = 8.4 Hz, 1H), 8.04-7.91 (m, 4H), 7.50-7.44 (m, 2H), 7.43-7.36 (m, 2H), 7.30-7.19 (m, 3H), 6.90 (t, J = 5.2 Hz, 1H), 6.51-6.41 (m, 2H), 3.25-3.19 (m, 2H), 2.75-2.65 (m, 1H), 2.26-2.15 (m, 1H), 2.13-2.03 (m, 1H), 1.89 (s, 3H), 1.85-1.73 (m, 3H), 1.50-1.33 (m, 2H). | MS ESI calcd. For 519.24 [M + H]⁺ 520.24, found 520.4. |

### Example 219 and 220. Synthesis of (1S,4s)-4-(((R)-3-(4-(2-(2-aminopyridin-3-yl)-5-(2-fluorophenyl)-3H-imidazo[4,5-b]pyridin-3-yl)phenyl)pyrrolidin-1-yl)methyl)cyclohexane-1-carboxylic acid

### Isomer 1: Compound I-228

### Isomer 2: Compound I-229

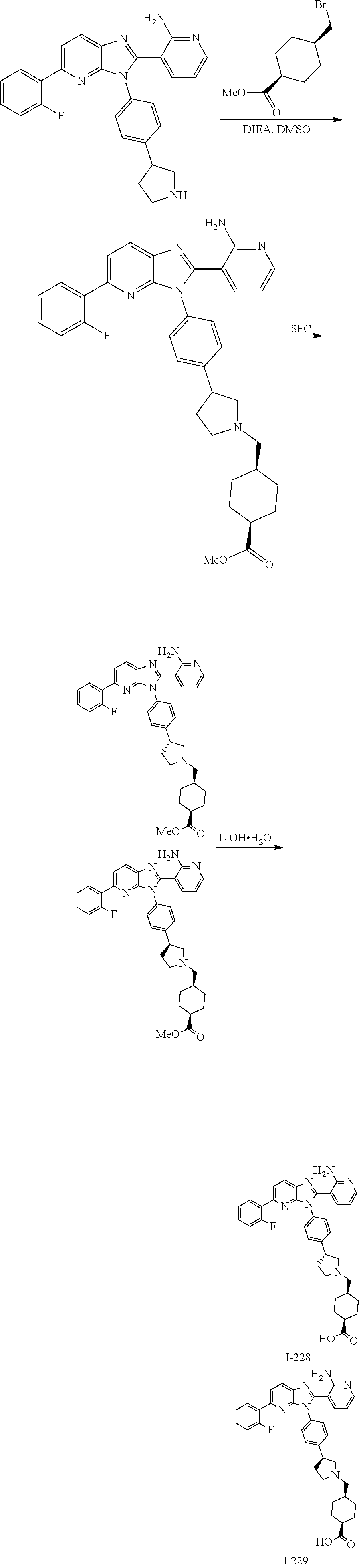

Step 1. To the solution of 3-[5-(2-fluorophenyl)-3-(4-pyrrolidin-3-ylphenyl)imidazo[4,5-b]pyridin-2-yl]pyridin-2-amine (200 mg, 0.444 mmol) in DMSO (3 mL) was added cis-methyl 4-(bromomethyl)cyclohexanecarboxylate (157 mg, 0.666 mmol) and DIEA (0.22 mL, 1.33 mmol) then stirred at 80° C. for 16 hrs under N₂. The reaction mixture was added H₂O (100 ml) and extracted with EtOAc (3×40 mL). The organic layer was washed with brine and dried over Na₂SO₄ and concentrated in vacuum to give a crude. The crude was purified by flash column (0-30% of MeOH in DCM) to give cis-methyl 4-[[3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylate (230 mg) as a yellow solid. The compound cis-methyl 4-[[3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylate was purified by prep-SFC (column: DAICEL CHIRALPAK AD 250 mm*30 mm, 10 um; mobile phase:CO2-i-PrOH(0.1% NH₃H₂O); B %:45%-45%, 150 min).

Isomer 1. cis-methyl 4-[[(3R)-3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylate (90.0 mg, 0.149 mmol, 34% yield) as white solid. ¹H NMR (400 MHz, DMSO-d₆) δ=8.28 (d, J=8.4 Hz, 1H), 8.00 (dd, J=2.0, 4.8 Hz, 1H), 7.83-7.74 (m, 2H), 7 50-7.37 (m, 5H), 7.36-7.28 (m, 2H), 7.19 (dd, J=1.6, 7.6 Hz, 1H), 6.94 (s, 2H), 6.41 (dd, J=5.2, 7.6 Hz, 1H), 3 59 (s, 3H), 3 44-3.35 (m, 1H), 2.97-2.81 (m, 1H), 2.70-2.59 (m, 2H), 2.36-2.18 (m, 3H), 1.90-1.72 (m, 3H), 1.65-1.44 (m, 5H), 1.27-1 11 (m, 2H), 1.04 (d, J=6.0 Hz, 2H).

Isomer 2. cis-methyl 4-1[(3S)-3-[4-[2-(2-amino-3-pyridyl)-5-(2-fluorophenyl)imidazo[4,5-b]pyridin-3-yl]phenyl]pyrrolidin--yl]methyl]cyclohexanecarboxylate (78.0 mg, 0.129 mmol, 29% yield) as a white solid. ¹H NMR (400 MHz, DMSO-d₆) δ=8.28 (d, J=8.4 Hz, 1H), 8.03-7 97 (m, 1H), 7.84-7.73 (m, 2H), 7.51-7.38 (m, 5H), 7.36-7.27 (m, 2H), 7.25-7.16 (m, 1H), 6.94 (s, 2H), 6.41 (dd, J=4.8, 7.6 Hz, 1H), 3.59 (s, 3H), 2.67 (s, 2H), 2.39-2.23 (m, 3H), 1.92-1.73 (m, 3H), 1.63-1.44 (m, 5H), 1.25-1.13 (m, 3H), 1.04 (d, J=6.0 Hz, 3H).

Step 2. To the solution of cis-methyl 4-[[(3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylate (90.0 mg, 0.149 mmol) in water (1 mL) and THF (1 mL) was added LIOH·H₂O (25.0 mg, 0.595 mmol) then stirred at 25° C. for 16 hrs. The reaction mixture was purified by prep-HPLC (column: Xtimate C18 150*30 mm*5 um, mobile phase: water (FA)-ACN, B %. 12%-42%, 30 min) to give

Isomer 1. cis-4-[[(3R)-3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylic acid I-228 (34.0 mg, 0.0564 mmol, 37.9% yield) as a yellow solid. ¹H NMR (400 MHz, DMSO-d₆) δ=8 31-8.23 (m, 1H), 7.99 (dd, J=1.6, 4.8 Hz, 1H), 7.82-7.70 (m, 2H), 7.52-7.36 (m, 5H), 7.35-7.26 (m, 2H), 7.19 (dd, J=1.6, 7.6 Hz, 1H), 6.41 (dd, J=4.8, 7.6 Hz, 1H), 3.44-3.33 (m, 1H), 3.03 (t, J=8.8 Hz, 1H), 2.82-2.71 (m, 2H), 2.60-2.55 (m, 1H), 2.47-2.35 (m, 3H), 2.34-2.21 (m, 1H), 1.89-1.75 (m, 3H), 1.63-1.42 (m, 5H), 1.26-1.11 (m, 2H). HPLC Rt=3.732 min in 15 min chromatography, purity 99%. LCMS Rₜ=2.666 min in 4 min chromatography, purity 99.5%, MS ESI calcd. For 590.28 [M+H]+ 591.28. found 591.4.

Isomer 2. cis-4-[[(3S)-3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylic acid I-229 (33.0 mg, 0.0554 mmol, 43% yield) as a yellow solid. ¹H NMR (400 MHz, DMSO-d₆) δ=8.28 (d, J=8.4 Hz, 1H), 7.99 (dd, J=1.6, 4.8 Hz, 1H), 7.81-7.74 (m, 2H), 7.49-7.36 (m, 5H), 7.35-7.26 (m, 2H), 7.19 (dd, J=2.0, 7.6 Hz, 1H), 6.41 (dd, J=4.8, 7.6 Hz, 1H), 3.42-3.34 (m, 2H), 2.99-2.89 (m, 1H), 2.75-2.62 (m, 2H), 2.46-2.41 (m, 1H), 2.40-2.23 (m, 3H), 1.89-1.72 (m, 3H), 1.65-1.40 (m, 5H), 1.25-1.13 (m, 2H). HPLC Rt=3.742 min in 15 min chromatography, purity 99.1%. LCMS Rₜ=2.665 min in 4 min chromatography, purity 99.3%, MS ESI calcd. For 590.28 [M+H]+ 591.28, found 591.4.

### Example 221 and 222. Synthesis of (trans-4-((3-(4-(2-(2-aminopyridin-3-yl)-5-(2-fluorophenyl)-3H-imidazo[4,5-b]pyridin-3-yl)phenyl)pyrrolidin-1-yl)methyl)cyclohexane-1-carboxylic acid

### Isomer 1. Compound I-230

### Isomer 2. Compound I-231

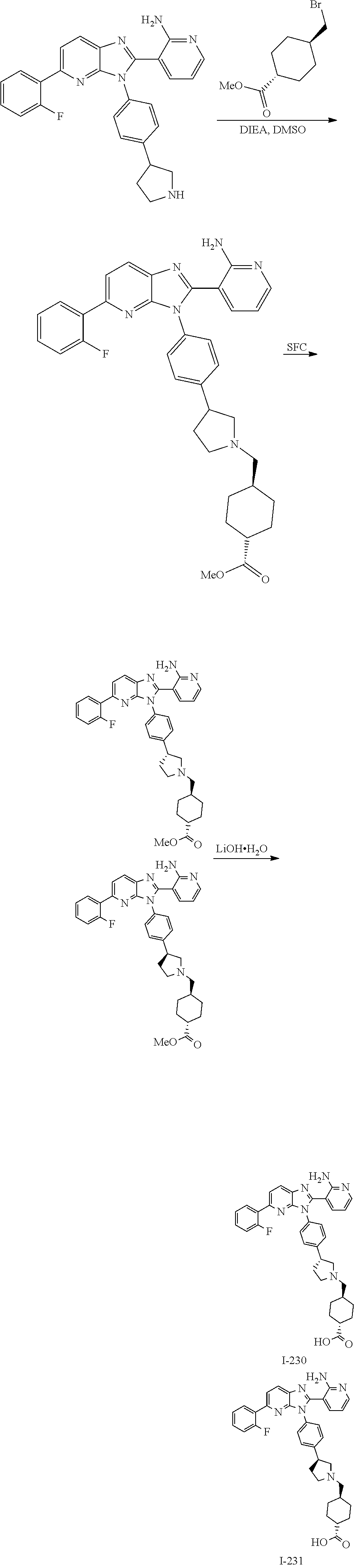

Step 1. To the solution of 3-[5-(2-fluorophenyl)-3-(4-pyrrolidin-3-ylphenyl)imidazo[4,5-b]pyridin-2-yl]pyridin-2-amine (400 mg, 0.888 mmol) in DMSO (3 mL) was added trans-methyl 4-(bromomethyl)cyclohexanecarboxylate (313 mg, 1 33 mmol) and DIEA (0.44 mL, 2.66 mmol) then stirred at 80° C. for 16 hrs under N₂. The reaction mixture was added H₂O (100 ml) and extracted with EtOAc (3×40 mL). The organic layer was washed with brine then dried over Na₂SO₄ and concentrated in vacuum to give a crude. The crude was purified by flash column (0-20% of MeOH in DCM) to give compound trans-methyl 4-[[3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylate (470 mg). The product was purified by prep-SFC (column: DAICEL CHIRALPAK AD 250 mm*30 mm, 10 um; mobile phase:CO₂-i-PrOH(0.1% NH₃H₂O); B %: 45%-45%, 150 min)

Isomer 1 methyl 4-[[(3R)-3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylate (130 mg, 0.215 mmol, 24% yield) as yellow solid. ¹H NMR (400 MHz, DMSO-d₆) δ=8.28 (d, J=8.0 Hz, 1H), 8.00 (dd, J=1.6, 4.8 Hz, 1H), 7.83-7.75 (m, 2H), 7.50-7.37 (m, 5H), 7.36-7.27 (m, 2H), 7.19 (dd, J=1.6, 7.6 Hz, 1H), 6.94 (s, 2H), 6.41 (dd, J=4.4, 7.2 Hz, 1H), 3.58 (s, 3H), 3.41-3.34 (m, 1H), 2.89 (t, J=8.4 Hz, 1H), 2.70-2.58 (m, 2H), 2.48-2 42 (m, 1H), 2.35-2.16 (m, 4H), 1.92-1.72 (m, 5H), 1.49-1.36 (m, 1H), 1.35-1.24 (m, 2H), 0.96-0.80 (m, 2H).

Isomer 2 trans-methyl 4-[[(3S)-3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylate (70.0 mg, 0.116 mmol, 58% yield) as a yellow solid. ¹H NMR (400 MHz, DMSO-d₆) δ=8.28 (d, J=8.4 Hz, 1H), 8.00 (dd, J=1.6, 4.8 Hz, 1H), 7.82-7.74 (m, 2H), 7.51-7.37 (m, 5H), 7.36-7.27 (m, 2H), 7.19 (dd, J=1.6, 7.6 Hz, 1H), 6.94 (s, 2H), 6.41 (dd, J=4.8, 7.6 Hz, 1H), 3.58 (s, 3H), 3.39 (s, 1H), 2.89 (t, J=8.4 Hz, 1H), 2.69-2.58 (m, 2H), 2.48-2.43 (m, 1H), 2.36-2.16 (m, 4H), 1.93-1.73 (m, 5H), 1.49-1.38 (m, 1H), 1.35-1.24 (m, 2H), 0.98-0.80 (m, 2H).

Step 2. To the solution of trans-methyl 4-[[(3R)-3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylate (130 mg, 0.215 mmol) in water (1 mL) and THF (1 mL) was added LiOH·H₂O (36.0 mg, 0.860 mmol) then stirred at 25° C. for 16 hrs. The reaction mixture was purified by prep-HPLC (column: Xtimate C18 150*30 mm*5 um; mobile phase: water (FA)-ACN; B %: 12%-42%, 30 min) to give

Isomer 1 trans-4-[[(3R)-3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylic acid I-230 (70.0 mg, 0.117 mmol, 55% yield) as a yellow solid. ¹H NMR (400 MHz, DMSO-d₆) δ=8.30-8.22 (m, 1H), 7.99 (dd, J=2.0, 4.8 Hz, 1H), 7.79-7.73 (m, 2H), 7.48-7.35 (m, 5H), 7.34-7.26 (m, 2H), 7.19 (dd, J=2.0, 7.6 Hz, 1H), 6.41 (dd, J=5.2, 8.0 Hz, 1H), 3.44-3.34 (m, 1H), 3 00 (t, J=8.8 Hz, 1H), 2.73 (t, J=7.6 Hz, 2H), 2.57-2.53 (m. IH), 2.41-2.24 (m, 3H), 2.17-2.07 (m, 1H), 1.91-1.73 (m, 5H), 1.48-1.36 (m, 1H), 1.33-1.21 (m, 2H), 0.97-0.82 (m, 2H). HPLC Rt=3.604 min in 15 min chromatography, purity 99.6%. LCMS Rₜ=2.590 min in 4 min chromatography, purity 99.5%, MS ESI calcd. For 590.28 [M+H]+ 591.28, found 591.4.

Isomer 2 trans-4-[[(3S)-3-[4-[2-(2-amino-3-pyridyl)-5-(2-fluorophenyl)imidazo[4,5-b]pyridin-3-yl]phenyl]pyrrolidin-1-yl]methyl]cyclohexanecarboxylic acid I-231 (35.0 mg, 0.0580 mmol, 50% yield) as a yellow solid. ¹H NMR (400 MHz, DMSO-d₆) δ=8.27 (d, J=8.4 Hz, 1H), 7.98 (dd, J=2.0, 4.8 Hz, 1H), 7.79-7.72 (m, 2H), 7.48-7.35 (m, 5H), 7.34-7.24 (m, 2H), 7.19 (dd, J=1.6, 7.6 Hz, 1H), 6.41 (dd, J=4 8, 7.6 Hz, 1H), 3.45-3.33 (m, 1H), 3.00 (t, J=8.8 Hz, 1H), 2.79-2.64 (m, 2H), 2.56-2.52 (m, 1H), 2.41-2.22 (m, 3H), 2.16-2.05 (m, 1H), 1.91-1.74 (m, 5H), 1.49-1.36 (m, 1H), 1 34-1.18 (m, 2H), 0.97-0.81 (m, 2H). HPLC Rt=3.634 min in 8 min chromatography, purity 99.0%. LCMS Rₜ=2.646 min in 4 min chromatography, purity 98.1%, MS ESI calcd. For 590.28 [M+H]+ 591.28, found 591.5.

### Example 223 and 224. Synthesis of trans-4-((3-(4-(2-(2-aminopyridin-3-yl)-5-phenyl-3H-imidazo[4,5-b]pyridin-3-yl)phenyl)pyrrolidin-1-yl)methyl)cyclohexane-1-carboxylic acid

### Isomer 1 (Compound I-232)

### Isomer 2 (Compound I-233)

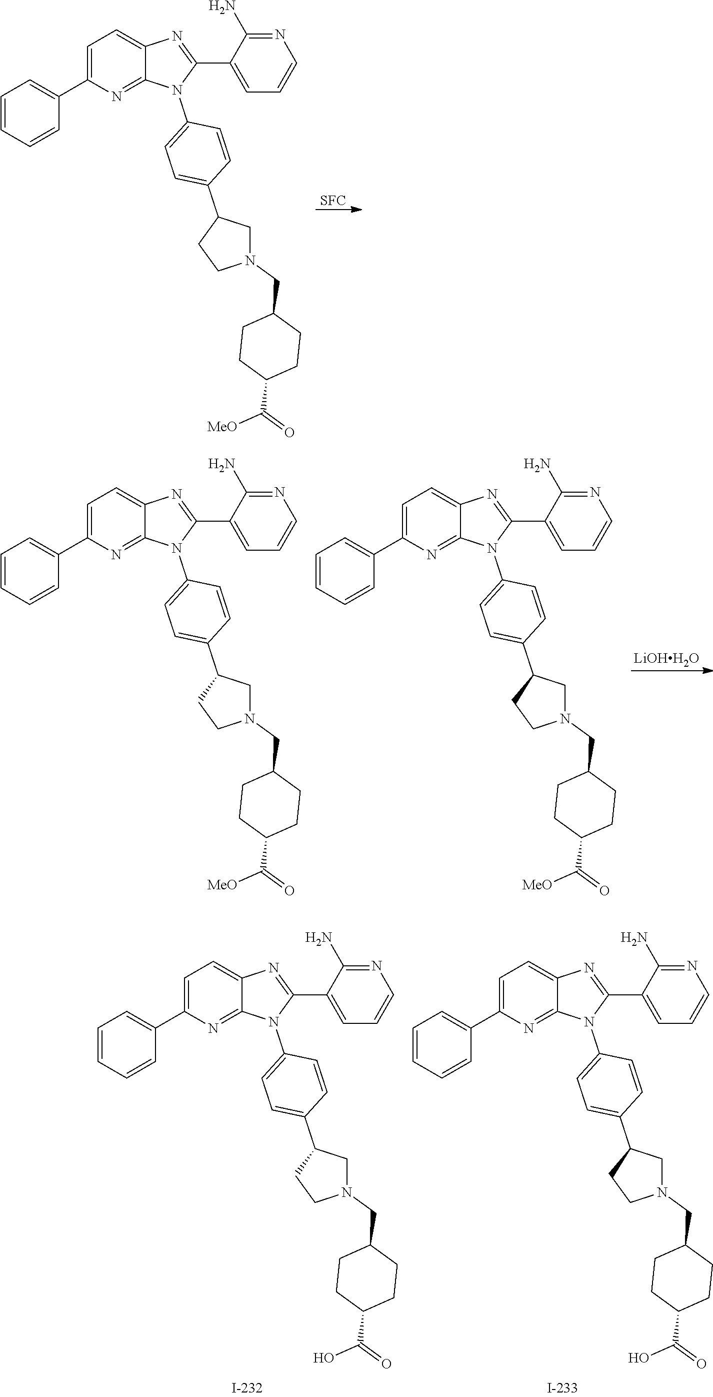

Step 1. Trans-methyl 4-[[3-[4-[2-(2-amino-3-pyridyl)-5-phenyl-imidazo[4,5-b]pyridin-3-yl]phenyl]pyrrolidin-1-yl]methyl]cyclohexanecarboxylate (700 mg, 1.19 mmol) was purified by SFC (Column DAICEL CHIRALPAK AD (250 mm*30 mm,10 um). Condition CO₂-i-PrOH (0.1% NH₃H₂O) Begin B 50% End B 50%)

Isomer 1: trans-methyl 4-[[(3R)-3-[4-[2-(2-amino-3-pyridyl)-5-phenyl-imidazo[4,5-b]pyridin-3-yl]phenyl]pyrrolidin-1-yl]methyl]cyclohexanecarboxylate (260 mg, 0.443 mmol, 37% yield) as a yellow solid. ¹H NMR (CHLOROFORM-d 400 MHz) δ_{H}=8.12 (d, J=8.4 Hz, 1H), 8.07 (dd, J=1.6, 4.8 Hz, 1H), 8.04-7.99 (m, 2H), 7.79 (d, J=8.4 Hz, 1H), 7.47-7.41 (m, 4H), 7.40-7.34 (m, 3H), 7.12 (dd, J=1.6, 7.6 Hz, 1H), 6.59 (br s, 2H), 6.39 (dd, J=4.8, 7 6 Hz, IH), 3.66 (s, 3H), 3.52-3.39 (m, 1H), 3.03 (br dd, J=6.4, 8.8 Hz, 1H), 2.85-2.53 (m, 3H), 2.46-2.20 (m, 4H), 2.08-1.94 (m, 4H), 1.80-1.61 (m, 1H), 1.54-1.37 (m, 3H), 1.04-0.87 (m, 2H). 0

Isomer 2: trans-methyl 4-[[(3S)-3-[4-[2-(2-amino-3-pyridyl)-5-phenyl-imidazo[4,5-b]pyridin-3-yl]phenyl]pyrrolidin-1-yl]methyl]cyclohexanecarboxylate (250 mg, 0.426 mmol, 36% yield) as a yellow solid. ¹H NMR (CHLOROFORM-d 400 MHz) δ_{H}=8.12 (d, J=8.4 Hz, 1H), 8.07 (dd, J=1.6, 4.8 Hz, 1H), 8.02 (d, J=7.2 Hz, 2H), 7.79 (d, J=8.4 Hz, 1H), 7.47-7.40 (m, 4H), 7 40-7.34 (m, 3H), 7.12 (dd, J=1.6, 7.6 Hz, 1H), 6.58 (br s, 2H), 6.39 (dd, =4 8, 7.6 Hz, 1H), 3.67 (s, 3H), 3.53-3.36 (m, 1H), 3.15-2.93 (m, 1H), 2.86-2.52 (m, 3H), 2.46-2.20 (m, 4H), 2.05-1.93 (m, 4H), 1.61 (br s, 1H), 1.52-1.37 (m, 3H), 1.07-0.86 (m, 2H

Step 2. To a solution of trans-methyl 4-[[(3R)-3-[4-[2-(2-amino-3-pyridyl)-5-phenyl-imidazo[4,5-b]pyridin-3-yl]phenyl]pyrrolidin-1-yl]methyl]cyclohexanecarboxylate (100 mg, 0.170 mmol) in THF (2 mL) and water (1 mL) was added LiOH H₂O (21.0 mg, 0 511 mmol). The mixture was stirred at 25° C. for 16 hrs. The mixture was concentrated. The residue was diluted with IM HCl and purified by prep-HPLC (Welch Xtimate C18 150*30 mm*5 um, water(FA)-CAN) to give

Isomer 1: trans-4-[[(3R)-3-[4-[2-(2-amino-3-pyridyl)-5-phenyl-imidazo[4,5-b]pyridin-3-yl]phenyl]pyrrolidin-1-yl]methyl]cyclohexanecarboxylic acid 1-232 (63.9 mg, 0.112 mmol, 65.4% yield) as a white solid. ¹H NMR (DMSO-d₆, 400 MHz) δ_{H}=8.25 (d, J=8.4 Hz, 1H), 8.17 (s, 1H), 8.04-7.94 (m, 4H), 7.52-7.36 (m, 7H), 7.19 (dd, J=1.6, 7.6 Hz, 1H), 6.42 (dd, J=4.8, 7.6 Hz, 1H), 3.48 (s, 1H), 3.31 (br t, J=7 6 Hz, 1H), 3.01 (br s, 2H), 2.82 (t, J=8 0 Hz, 1H), 2.62 (s, 21), 2.43-2.29 (m, 1H), 2.19-2.07 (m, 1H), 1.98-1.81 (m, 5H), 1.55 (d, J=7.2 Hz, 1H), 1.38-1.23 (m, 2H), 1.02-0.86 (m, 2H). HPLC Rt=4.232 min in 8 min chromatography, 0-60AB_1_T3_COL2.M, purity 100% LCMS Rₜ=4.075 min in 7 min chromatography, 0-60AB_7 min_220&254_Shimadzu.1 cm, purity 100%, MS ESI calcd. for 572.21 [M+H]⁺ 573.21, found 573.2.

Isomer 2: trans-4-1[(3S)-3-[4-[2-(2-amino-3-pyridyl)-5-phenyl-imidazo[4,5-b]pyridin-3-yl]phenyl]pyrrolidin-1-yl]methyl]cyclohexanecarboxylic acid 1-233 (59.7 mg, 0.104 mmol, 61% yield) as a white solid. ¹H NMR (METHANOL-d4 400 MHz) δ_{H}=8.51 (s, 1H), 8.18 (d, J=8.4 Hz, 1H), 8.04-7.99 (m, 2H), 7.98 (dd, J=1.6, 5.2 Hz, 1H), 7.93 (d, J=8.4 Hz, 1H), 7.57-7.51 (m, 2H), 7.51-7.46 (m, 2H), 7.45-7.30 (m, 4H), 6.48 (dd, J=5.2, 7.6 Hz, 1H), 3.88-3.64 (m, 2H), 3.55-3.44 (m, 2H), 3.29-3.23 (m, 1H), 3.05 (d, J=6.8 Hz, 2H), 2.62-2.48 (m, 1H), 2.30-2.12 (m, 2H), 2.07-1.98 (m, 2H), 1.97-1.86 (m, 2H), 1.82-1.69 (m, 1H), 1.54-1.39 (m, 2H), 1.16-1.02 (m, 2H). HPLC Rt=4.220 min in 8 min chromatography, 0-60AB_1_T3_COL2.M, purity 99.8%. LCMS Rₜ=4.066 min in 7 min chromatography, 0-60AB_7 min_220&254_Shimadzu.1 cm, purity 100%, MS ESI calcd. for 572.21 [M+H]+ 573.21, found 573.2.

### Example 225. Synthesis of Cis-4-((3-(4-(2-(2-aminopyridin-3-yl)-6-methyl-5-phenyl-3H-imidazo[4,5-b]pyridin-3-yl)phenyl)azetidin-1-yl)methyl)cyclohexane-1-carboxylic acid (Compound I-234)

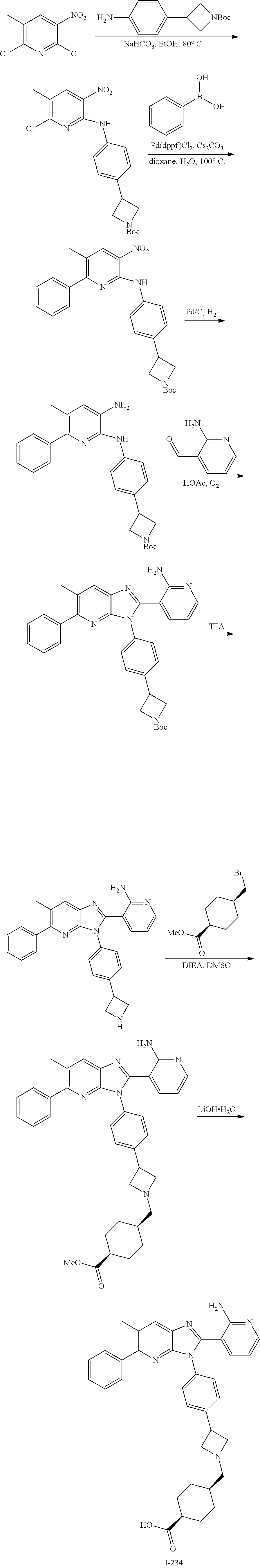

Step 1. To a solution of 2,6-dichloro-3-methyl-5-nitro-pyridine (1.00 g, 4.83 mmol) and tert-butyl 3-(4-aminophenyl)azetidine-1-carboxylate (1.20 g, 4.83 mmol) in ethanol (30 mL) was added NaHCO₃ (812 mg, 9.66 mmol). After stirring at 60° C. under N₂ for 4 h, the reaction was filtered, washed with DCM (30 mL*3) and the filtrate concentrated. The residue was purified by flash column (30-60% of DCM in PE) to give tert-butyl 3-[4-[(6-chloro-5-methyl-3-nitro-2-pyridyl)amino]phenyl]azetidine-1-carboxylate (540 mg, 1.29 mmol) as yellow oil. ¹H NMR (DMSO-d₆ 400 MHz) δ_{H}=9.97 (s, 1H), 8.56 (s, 1H), 7.59 (d, J=8.4 Hz, 2H), 7.35 (d, J=8.4 Hz, 2H), 4.39-4.14 (m, 2H), 3.94-3.72 (m, 3H), 2.30 (s, 3H), 1.41 (s, 9H).

Step 2. To a solution of tert-butyl 3-[4-[(6-chloro-5-methyl-3-nitro-2-pyridyl)amino]phenyl]azetidine-1-carboxylate (540 mg, 1.29 mmol) and phenylboronic acid (189 mg, 1.55 mmol) in 1,4-Dioxane (10 mL) and water (2 mL) was added Cs₂CO₃ (1.26 g, 3.87 mmol) and Pd(dppf)Cl₂ (94 0 mg, 0.129 mmol). The reaction was stirred at 100 C for 4 hrs under N₂. The reaction mixture was filtered and the water phase was separated. The organic layer was dried over Na₂SO₄ filtered and concentrated. The residue was purified by flash column (20% of EtOAc in PE) to give tert-butyl 3-[4-[(5-methyl-3-nitro-6-phenyl-2-pyridyl)amino]phenyl]azetidine-1-carboxylate (540 mg, 1.17 mmol) as yellow oil. ¹H NMR (DMSO-d₆ 400 MHz) δ_{H}=9.95 (s, 1-i), 8.52 (s, 1H), 7.71 (d, J=8.4 Hz, 2H), 7 67-7.60 (m, 2H), 7.56-7.44 (m, 3H), 7.29 (d, J=8.4 Hz, 2H), 4.28-4.14 (m, 2H), 3.89-3.73 (m, 3H), 2.33 (s, 3H), 1.39 (s, 9H).

Step 3. To a solution of tert-butyl 3-[4-[(5-methyl-3-nitro-6-phenyl-2-pyridyl)amino]phenyl]azetidine-1-carboxylate (540 mg, 1.17 mmol) in THF (10 mL) was added wet Pd/C (62 mg, 0.0586 mmol, 10%) under Ar. The suspension was degassed under vacuum and purged with Ar for three times. Then the suspension was degassed under vacuum and purged with H₂ for three times. The mixture was stirred at 20° C. under H₂ (15 psi) for 3 hrs. The mixture was filtered and washed with THF (3×10 mL). The filtrate was concentrated to give tert-butyl 3-[4-[(3-amino-5-methyl-6-phenyl-2-pyridyl)amino]phenyl]azetidine-1-carboxylate (500 mg, 1.16 mmol) as yellow oil. ¹H NMR (DMSO-d₆ 400 MHz) δ_{H}=7.75 (s, 1H), 7.67 (d, J=8.4 Hz, 2H), 7.54-7.48 (m, 2H), 7.43-7.36 (m, 2H), 7.32-7.25 (m, 1H), 7.16 (d, J=8.4 Hz, 2H), 6.82 (s, 1H), 5.13 (s, 2H), 4.30-4.09 (m, 2H), 3.83-3.64 (m, 3H), 2.20 (s, 3H), 1.40 (s, 9H).

Step 4. A mixture of tert-butyl 3-[4-[(3-amino-5-methyl-6-phenyl-2-pyridyl)amino]phenyl]azetidine-1-carboxylate (500 mg, 1.16 mmol) and 2-aminopyridine-3-carbaldehyde (170 mg, 1.39 mmol) in acetic acid (5 mL) was stirred at 90° C. under O₂ (15 mL) for 1 hr. The mixture was adjusted pH-9 with NaOH (2 M) and extracted with DCM (20 mL*2). The combined organic phase was washed with brine (50 mL), dried over Na₂SO₄, filtered and concentrated. The residue was purified by flash column (100% of EtOAc in PE) to give tert-butyl 3-[4-[2-(2-amino-3-pyridyl)-6-methyl-5-phenyl-imidazo[4,5-b]pyridin-3-yl]phenyl]azetidine-1-carboxylate (450 mg, 0.845 mmol) as a yellow solid. ¹H NMR (DMSO-d₆ 400 MHz) δ_{H}=8.14 (s, 1H), 8.03-7.93 (m, 1H), 7.55-7.33 (m, 9H), 7.21-7.12 (m, 1H), 6.97 (s, 2H), 6.48-6.33 (m, 1H), 4.34-4.17 (m, 2H), 3.95-3 76 (m, 3H), 2.42 (s, 3H), 1.39 (s, 9H).

Step 5. To a solution of tert-butyl 3-[4-[2-(2-amino-3-pyridyl)-6-methyl-5-phenyl-imidazo[4,5-b]pyridin-3-yl]phenyl]azetidine-1-carboxylate (450 mg, 0.845 mmol) in DCM (10 mL) was added TFA (3.0 mL). The mixture was stirred at 20° C. for 16 hrs. The mixture was adjust pH-~11 with NaOH (2 M) and extracted with DCM/MeOH(10/1) (3×50 mL). The combined organic phase was washed with brine(100 mL), dried over Na₂SO₄, filtered and concentrated to give 3-[3-[4-(azetidin-3-yl)phenyl]-6-methyl-5-phenyl-imidazo[4,5-b]pyridin-2-yl]pyridin-2-amine (350 mg, 0.809 mmol) as a yellow solid. ¹H NMR (DMSO-d₆, 400 MHz) δ_{H}=8.13 (s, 1H), 7.98 (dd, J=1.6, 4.8 Hz, 1H), 7.51-7.34 (m, 9H), 7.16 (dd, J=1.6, 7.6 Hz, 1H), 6.98 (s, 2H), 6.44-6.35 (m, 1H), 3.95-3.83 (m, 1H), 3.75 (t, J=7.6 Hz, 2H), 3.60 (t, J=7.6 Hz, 21), 2.42 (s, 3H)

Step 6. To a solution of 3-[3-[4-(azetidin-3-yl)phenyl]-6-methyl-5-phenyl-imidazo[4,5-b]pyridin-2-yl]pyridin-2-amine (350 mg, 0.809 mmol) and cis-methyl 4-(bromomethyl)cyclohexanecarboxylate (285 mg, 1.21 mmol) in DMSO (10 mL) was added DIEA (0.45 mL, 2.43 mmol). After stirring at 85° C. under N₂ for 16 hrs, the reaction mixture was poured into H₂O (20 mL) and extracted with EtOAc (3×30 mL). The organic layer was washed with brine (3×100 mL), dried over Na₂SO₄ and concentrated. The residue was purified by flash column (0~5% of MeOH in DCM) to give cis-methyl 4-[[3-[4-[2-(2-amino-3-pyridyl)-6-methyl-5-phenyl-imidazo[4,5-b]pyridin-3-yl]phenyl]azetidin-1-yl]methyl]cyclohexanecarboxylate (180 mg, 0.307 mmol) as a yellow solid. ¹H NMR (DMSO-d₆ 400 MHz) δ_{H}=8.13 (s, 1H), 7.98 (dd, J=2.0, 4.8 Hz, 1H), 7.54-7.32 (m, 9H), 7.16 (dd, J=1.6, 7.6 Hz, 1H), 6.96 (s, 2H), 6.46-6.35 (m, 1H), 3.59 (s, 6H), 3.04 (br t, J=5.6 Hz, 2H), 2.42 (s, 3H), 2.28 (d, J=7.2 Hz, 2H), 1.89-1 76 (m, 2H), 1.58-1.43 (m, 4H), 1.42-1.32 (m, 1H), 1.27-1.07 (m, 3H).

Step 7. To the solution of cis-methyl 4-[[3-[4-[2-(2-amino-3-pyridyl)-6-methyl-5-phenyl-imidazo[4,5-b]pyridin-3-yl]phenyl]azetidin-1-yl]methyl]cyclohexanecarboxylate (180 mg, 0.307 mmol) in water (2 mL) and THF (5 mL) was added LiOH·H₂O (39.0 mg, 0.920 mmol) then stirred at 25° C. for 16 hrs. THT was removed and the residue was purified by prep-HPLC(column: Xtimate C18 150*40 mm*5 um; mobile phase:water (FA)-ACN; B %:33%-63%, 60 min) to give cis-4-[[3-[4-[2-(2-amino-3-pyridyl)-6-methyl-5-phenyl-imidazo[4,5-b]pyridin-3-yl]phenyl]azetidin-1-yl]methyl]cyclohexanecarboxylic acid I-234 (124 mg, 0.216 mmol, 70% yield) as a yellow solid. ¹H NMR (DMSO-d₆ 400 MHz) δ_{H}=8.19 (s, 1H), 8.13 (s, 1H), 7.98 (dd, J=1.6, 4 8 Hz, 1H), 7.52-7.33 (m, 9H), 7 16 (dd, J=2.0, 7.6 Hz, 1H), 6.96 (s, 2H), 6.46-6.35 (m, 1H), 3.65-3.61 (m, 3H), 3.13-3.10 (m, 2H), 2.42 (s, 4H), 2.33 (br d, J=7.2 Hz, 2H), 1.85-1.75 (m, 2H), 1.56-1.36 (m, 5H), 1.25-1.13 (m, 2H). HPLC Rₜ=3.940 min in 8 min chromatography, 0-60AB, purity 99.3%. LCMS Rₜ=2.776 in 4 min chromatography, ShimNex HE C18-AQ 3 μm, 3.0*30 mm, purity 100%, MS ESI calcd. for 572.3 [M+H]⁺ 573.3, found 573.2.

### Example 226. Synthesis of trans-4-((3-(4-(2-(2-aminopyridin-3-yl)-6-methyl-5-phenyl-3H-imidazo[4,5-b]pyridin-3-yl)phenyl)azetidin-1-yl)methyl)cyclohexane-1-carboxylic acid (Compound I-235)

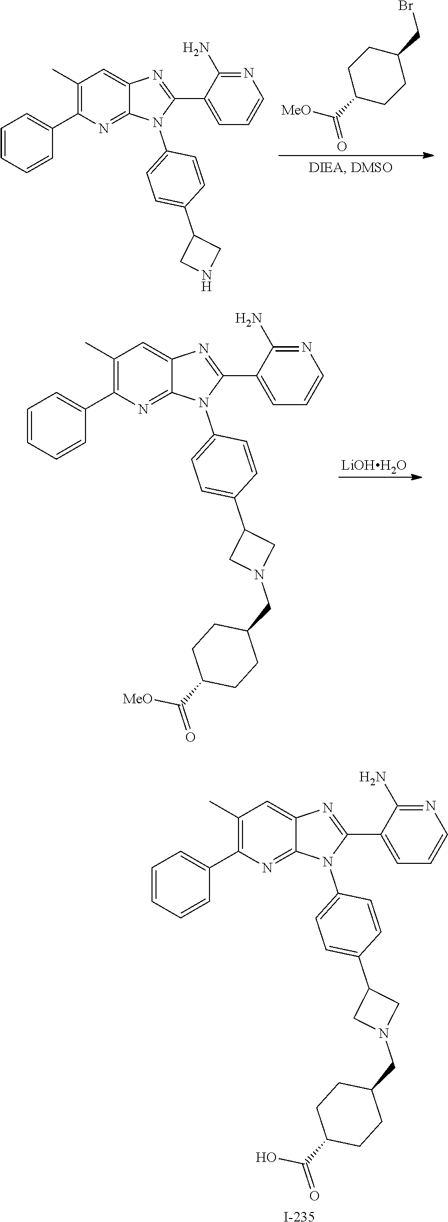

Step 1. To a solution of 3-[3-[4-(azetidin-3-yl)phenyl]-6-methyl-5-phenyl-imidazo[4,5-b]pyridin-2-yl]pyridin-2-amine (250 mg, 0.578 mmol) and trans-methyl 4-(bromomethyl)cyclohexanecarboxylate (204 mg, 0.867 mmol) in DMSO (10 mL) was added DIEA (0.32 mL, 1 73 mmol). After stirring at 85° C. under N₂ for 16 hrs The reaction mixture was poured into H₂O (20 mL) and extracted with EtOAc (3×30 mL). The organic layer was washed with brine (3×100 mL), dried over Na₂SO₄ and concentrated. The residue was purified by flash column (0-5% of MeOH in DCM) to give trans-methyl 4-[[3-[4-[2-(2-amino-3-pyridyl)-6-methyl-5-phenyl-imidazo[4,5-b]pyridin-3-yl]phenyl]azetidin-1-yl]methyl]cyclohexanecarboxylate (200 mg, 0.341 mmol) as a yellow solid. ¹H NMR (DMSO-d₆ 400 MHz) δ_{H}=8.13 (s, 1H), 7.98 (dd, J=2.0, 4.8 Hz, 1H), 7.49-7.34 (m, 9H), 7.15 (dd, J=2.0, 7.6 Hz, 1H), 6.96 (s, 2H), 6.43-6.35 (m, 1H), 3.63-3.52 (m, 614), 3.06 (br s, 2H), 2.41 (s, 3H), 2.30-2.21 (m, 2H), 1.91-1.82 (m, 2H), 1.81-1.71 (m, 2H), 1.39-1.13 (m, 4H), 0.96-0.82 (m, 2H).

Step 2. To the solution of trans-methyl 4-[[3-[4-[2-(2-amino-3-pyridyl)-6-methyl-5-phenyl-imidazo[4,5-b]pyridin-3-yl]phenyl]azetidin-1-yl]methyl]cyclohexanecarboxylate (200 mg, 0.341 mmol) in THF (5 mL) and water (2 mL) was added LiOH·H₂O (43 mg, 1.02 mmol) then stirred at 25° C. for 16 hrs. THT was removed and the residue was purified by prep-HPLC (column: Xtimate C18 150*40 mm*5 μm; condition:water (FA)-ACN; B:15%-35%) to give trans-4-[[3-[4-[2-(2-amino-3-pyridyl)-6-methyl-5-phenyl-imidazo[4,5-b]pyridin-3-yl]phenyl]azetidin-1-yl]methyl]cyclohexanecarboxylic acid I-235 (130 mg, 0.227 mmol, 67% yield) as a yellow solid. ¹H NMR (DMSO-d₆ 400 MHz) δ_{H}=8.19 (s, 1H), 8 13 (s, 1H), 7.98 (dd, J=1.6, 4.8 Hz, 1H), 7.50-7.35 (m, 9H), 7.16 (dd, J=1.6, 7.6 Hz, 1H), 6.97 (s, 2H), 6.43-6.36 (m, 1H), 3.64-3.56 (m, 3H), 3.11-3.05 (m, 2H), 2.42 (s, 31H), 2.27 (d, J=6.8 Hz, 2H), 2.15-2.03 (m, 1H), 1.93-1.82 (m, 2H), 1.81-1.72 (m, 2H), 1.31-1.17 (m, 3H), 0.95-0.81 (m, 2H). HPLC Rₜ=3.854 min in 8 min chromatography, 0-60AB, purity 100%. LCMS Rₜ=2.734 in 4 min chromatography, ShimNex HE C18-AQ 3 μm, 3.0*30 mm, purity 100%, MS ESI calcd. for 572.3 [M+H]⁺ 573.3, found 573.1.

### Example 227. Biological Activity Assay for Exemplary Compounds

AKT1 E17K kinase assay: Inhibitory effects of compounds on AKT1 E17K were undertaken in a 10-pt dose response kinase activity assay measuring phospho-peptide product formation based on TR-FRET signal as described in the following paragraph.

Purified human recombinant kinase full-length AKT1 E17K was expressed in insect cells and activated in vitro by PDPK1. Ulight™-CREBtide (PerkinElmer, catalog number TRF0107) was used as the peptide substrate. The reaction buffer consisted of 50 mM HEPES pH 7.5, 10 mM MgCl₂, 0.01% Triton X-100, 0.01% BSA, 2 mM DTT, 0.6 nM AKT1 E17K, 50 nM Ulight™-CREBtide and either 50 μM ATP or 2 mM ATP. The compound solution was prepared as 10 mM DMSO stock. For the assay, a 1.3 serial dilution 10-pt dose response of test compounds, in duplicate, for each concentration were dispensed into a 384-well plate (Corning, catalog number 4513). Final DMSO concentration in the assay was 1%. The enzyme mixture (5 μL, 1.2 nM) were added and the mixture was incubated for 15 min at RT prior to the start of the kinase reaction by the addition of 5 μL substrate mixture (100 nM Ulight™-CREBtide). The reaction was stopped after 60 min of incubation at RT by the addition of 10 μL of Detection Mix (PerkinElmer, #CR97-100) consisting of Europium-anti-phospho-CREB (Ser133) (Perkin Elmer, catalog number TRF0200). Incubation with the detection mix for a further 60 min allowed binding of antibody to the phospho-Ulight™ CREBtide. The fluorescence emissions at 620 nm and 665 nm after excitation at 350 nm was measured using Envision reader. The ratio of the emissions at 665 nm and at 620 nm provided the measure for the amount of phosphorylated CREBtide product of AKT1 E17K kinase activity. To determine the IC₅₀ values, the data was normalized (enzyme reaction without inhibitor=0% inhibition, all other assay components but no enzyme=100% inhibition) and IC₅₀ values were calculated by a 4-parameter fit using an in-house developed protocol.

Table 9 assigns each compound a code for potency in the AKT1 E 17K kinase assay: A, B, or C. According to the code, A represents an IC₅₀ value <500 nM; B represents an IC₅₀ value from ≥500 nM to <2000 nM; and C represents an IC₅₀ value ≥2000 nM.

TABLE 9

| | AKT1 E17K |
|---|---|
| Compound No. | Lance IC₅₀ |
| I-1 | A |
| I-2 | A |
| I-3 | A |
| I-4 | A |
| I-5 | B |
| I-6 | B |
| I-7 | C |
| I-8 | B |
| I-9 | B |
| I-10 | A |
| I-11 | A |
| I-12 | A |
| I-13 | C |
| I-14 | C |
| I-15 | C |
| I-16 | A |
| I-17 | B |
| I-18 | C |
| I-19 | C |
| I-20 | C |
| I-21 | C |
| I-22 | C |
| I-23 | C |
| I-24 | C |
| I-25 | B |
| I-26 | C |
| I-27 | B |
| I-28 | C |
| I-29 | B |
| I-30 | C |
| I-31 | B |
| I-32 | B |
| I-33 | C |
| I-34 | C |
| I-35 | C |
| I-36 | B |
| I-37 | A |
| I-38 | A |
| I-39 | C |
| I-40 | A |
| I-41 | A |
| I-43 | A |
| I-46 | A |
| I-47 | A |
| I-48 | A |
| I-52 | A |
| I-53 | A |
| I-57 | A |
| I-58 | A |
| I-59 | A |
| I-60 | B |
| I-61 | A |
| I-62 | B |
| I-63 | B |
| I-64 | C |
| I-65 | C |
| I-66 | B |
| I-67 | C |
| I-68 | C |
| I-69 | A |
| I-70 | A |
| I-71 | A |
| I-72 | C |
| I-73 | C |
| I-74 | C |
| I-75 | A |
| I-76 | B |
| I-77 | C |
| I-78 | B |
| I-79 | C |
| I-80 | C |
| I-81 | C |
| I-82 | C |
| I-83 | C |
| I-84 | C |
| I-85 | C |
| I-86 | B |
| I-87 | C |
| I-88 | C |
| I-89 | C |
| I-90 | A |
| I-91 | A |
| I-92 | C |
| I-93 | B |
| I-94 | A |
| I-95 | A |
| I-96 | A |
| I-97 | B |
| I-98 | C |
| I-99 | C |
| I-100 | B |
| I-101 | C |
| I-102 | B |
| I-103 | C |
| I-104 | B |
| I-105 | B |
| I-106 | B |
| I-107 | C |
| I-108 | B |
| I-109 | B |
| I-110 | C |
| I-111 | B |
| I-112 | A |
| I-113 | B |
| I-114 | B |
| I-115 | A |
| I-116 | B |
| I-117 | C |
| I-118 | A |
| I-119 | B |
| I-120 | B |
| I-121 | A |
| I-122 | A |
| I-123 | C |
| I-124 | C |
| I-125 | A |
| I-126 | C |
| I-127 | B |
| I-128 | B |
| I-129 | B |
| I-130 | C |
| I-131 | B |
| I-132 | B |
| I-133 | C |
| I-134 | B |
| I-135 | C |
| I-136 | B |
| I-137 | A |
| I-138 | A |
| I-139 | A |
| I-140 | B |
| I-141 | B |
| I-142 | B |
| I-143 | B |
| I-144 | A |
| I-145 | B |
| I-146 | A |
| I-147 | A |
| I-148 | A |
| I-149 | A |
| I-150 | A |
| I-151 | A |
| I-152 | B |
| I-153 | A |
| I-154 | B |
| I-155 | A |
| I-156 | B |
| I-157 | B |
| I-158 | B |
| I-159 | A |
| I-160 | B |
| I-161 | A |
| I-162 | C |
| I-163 | C |
| I-164 | B |
| I-165 | A |
| I-166 | C |
| I-167 | B |
| I-168 | C |
| I-169 | A |
| I-170 | A |
| I-171 | A |
| I-172 | A |
| I-173 | B |
| I-174 | B |
| I-175 | B |
| I-176 | B |
| I-177 | B |
| I-178 | C |
| I-179 | B |
| I-180 | C |
| I-181 | B |
| I-182 | A |
| I-183 | C |
| I-184 | B |
| I-185 | B |
| I-186 | C |
| I-187 | C |
| I-188 | C |
| I-189 | C |
| I-190 | B |
| I-191 | B |
| I-192 | B |
| I-193 | C |
| I-194 | B |
| I-195 | B |
| I-196 | C |
| I-197 | C |
| I-198 | A |
| I-199 | A |
| I-200 | B |
| I-201 | A |
| I-202 | A |
| I-203 | A |
| I-204 | A |
| I-205 | B |
| I-206 | B |
| I-207 | B |
| I-208 | B |
| I-209 | B |
| I-210 | B |
| I-211 | C |
| I-212 | B |
| I-213 | A |
| I-214 | A |
| I-215 | B |
| I-216 | C |
| I-217 | B |
| I-218 | B |
| I-219 | B |
| I-220 | B |
| I-221 | B |
| I-222 | B |
| I-223 | C |
| I-224 | C |
| I-225 | A |
| I-226 | B |
| I-227 | B |
| I-228 | B |
| I-229 | B |
| I-230 | B |
| I-231 | A |
| I-232 | B |
| I-233 | A |
| I-234 | A |
| I-235 | A |

### Example 228. AKT1 Kinase Biological Activity Assay for Exemplary Compounds

AKT1 kinase assay: Inhibitory effects of compounds on wildtype AKT1 were undertaken in a 10-pt dose response kinase activity assay measuring phospho-peptide product formation based on TR-FRET signal as described in the following paragraph.

Purified human recombinant kinase full-length wildtype AKT1 was expressed in insect cells and activated in vitro by PDPK1. Ulight™-CREBtide (PerkinElmer, catalog number TRF0107) was used as the peptide substrate. The reaction buffer consisted of 50 mM HEPES pH 7.5, 10 mM MgCl₂, 0.01% Triton X-100, 0.01% BSA, 2 mM DTT, 0.6 nM AKT1,50 nM Ulight™-CREBtide and either 50 μM ATP or 2 mM ATP. The compound solution was prepared as 10 mM DMSO stock. For the assay, a 1:3 serial dilution 10-pt dose response of test compounds, in duplicate, for each concentration were dispensed into a 384-well plate (Corning, catalog number 4513). Final DMSO concentration in the assay was 1%. The enzyme mixture (5 μL, 1.2 nM) were added and the mixture was incubated for 15 min at RT prior to the start of the kinase reaction by the addition of 5 μL substrate mixture (100 nM Ulight™-CREBtide). The reaction was stopped after 60 min of incubation at RT by the addition of 10 μL of Detection Mix (PerkinElmer, #CR97-100) consisting of Europium-anti-phospho-CREB (Ser133) (Perkin Elmer, catalog number TRF0200). Incubation with the detection mix for a further 60 min allowed binding of antibody to the phospho-Ulight™ CREBtide. The fluorescence emissions at 620 nm and 665 nm after excitation at 350 nm was measured using Envision reader. The ratio of the emissions at 665 nm and at 620 nm provided the measure for the amount of phosphorylated CREBtide product of AKT1 kinase activity. To determine the IC₅₀ values, the data was normalized (enzyme reaction without inhibitor=0% inhibition, all other assay components but no enzyme=100% inhibition) and IC₅₀ values were calculated by a 4-parameter fit using an in-house developed protocol.

Results are provided in Table 10. The symbol +++ represents an IC₅₀ value <20 μM; the symbol ++ represents an IC₅₀ value from ≥20 μM to 80 μM.

TABLE 10

| | AKT1 Lance |
|---|---|
| Compound No. | IC₅₀ |
| I-1 | +++ |
| I-2 | +++ |
| I-3 | +++ |
| I-4 | +++ |
| I-5 | +++ |
| I-6 | +++ |
| I-7 | ++ |
| I-8 | +++ |
| I-9 | +++ |
| I-10 | +++ |
| I-11 | +++ |
| I-12 | +++ |
| I-13 | +++ |
| I-14 | +++ |
| I-15 | +++ |
| I-16 | +++ |
| I-17 | +++ |
| I-18 | +++ |
| I-19 | +++ |
| I-20 | +++ |
| I-21 | +++ |
| I-22 | ++ |
| I-23 | ++ |
| I-24 | +++ |
| I-25 | +++ |
| I-26 | +++ |
| I-27 | +++ |
| I-28 | +++ |
| I-29 | +++ |
| I-30 | +++ |
| I-31 | +++ |
| I-32 | +++ |
| I-33 | +++ |
| I-34 | +++ |
| I-35 | +++ |
| I-36 | +++ |
| I-37 | +++ |
| I-38 | +++ |
| I-39 | +++ |
| I-40 | +++ |
| I-41 | +++ |
| I-43 | +++ |
| I-46 | +++ |
| I-47 | +++ |
| I-48 | +++ |
| I-52 | +++ |
| I-53 | +++ |
| I-57 | +++ |
| I-58 | +++ |
| I-59 | +++ |
| I-60 | +++ |
| I-61 | +++ |
| I-62 | +++ |
| I-63 | +++ |
| I-64 | +++ |
| I-65 | +++ |
| I-66 | +++ |
| I-67 | +++ |
| I-68 | +++ |
| I-69 | +++ |
| I-70 | +++ |
| I-71 | +++ |
| I-72 | +++ |
| I-73 | +++ |
| I-74 | +++ |
| I-75 | +++ |
| I-76 | +++ |
| I-77 | +++ |
| I-78 | +++ |
| I-79 | +++ |
| I-80 | +++ |
| I-81 | +++ |
| I-82 | +++ |
| I-83 | +++ |
| I-84 | +++ |
| I-85 | +++ |
| I-86 | +++ |
| I-87 | +++ |
| I-88 | +++ |
| I-89 | +++ |
| I-90 | +++ |
| I-91 | +++ |
| I-92 | +++ |
| I-93 | +++ |
| I-94 | +++ |
| I-95 | +++ |
| I-96 | +++ |
| I-97 | +++ |
| I-98 | +++ |
| I-99 | +++ |
| I-100 | +++ |
| I-101 | +++ |
| I-102 | +++ |
| I-103 | +++ |
| I-104 | +++ |
| I-105 | +++ |
| I-106 | +++ |
| I-107 | +++ |
| I-108 | +++ |
| I-109 | +++ |
| I-110 | +++ |
| I-111 | +++ |
| I-112 | +++ |
| I-113 | +++ |
| I-114 | +++ |
| I-115 | +++ |
| I-116 | +++ |
| I-117 | +++ |
| I-118 | +++ |
| I-119 | +++ |
| I-120 | +++ |
| I-121 | +++ |
| I-122 | +++ |
| I-123 | +++ |
| I-124 | +++ |
| I-125 | +++ |
| I-126 | +++ |
| I-127 | +++ |
| I-128 | +++ |
| I-129 | +++ |
| I-130 | +++ |
| I-131 | +++ |
| I-132 | +++ |
| I-133 | +++ |
| I-134 | +++ |
| I-135 | +++ |
| I-136 | +++ |
| I-137 | +++ |
| I-138 | +++ |
| I-139 | +++ |
| I-140 | +++ |
| I-141 | +++ |
| I-142 | +++ |
| I-143 | +++ |
| I-144 | +++ |
| I-145 | +++ |
| I-146 | +++ |
| I-147 | +++ |
| I-148 | +++ |
| I-149 | +++ |
| I-150 | +++ |
| I-151 | +++ |
| I-152 | +++ |
| I-153 | +++ |
| I-154 | +++ |
| I-155 | +++ |
| I-156 | +++ |
| I-157 | +++ |
| I-158 | +++ |
| I-159 | +++ |
| I-160 | +++ |
| I-161 | +++ |
| I-162 | +++ |
| I-163 | +++ |
| I-164 | +++ |
| I-165 | +++ |
| I-166 | +++ |
| I-167 | +++ |
| I-168 | +++ |
| I-169 | +++ |
| I-170 | +++ |
| I-171 | +++ |
| I-172 | +++ |
| I-173 | +++ |
| I-174 | +++ |
| I-175 | +++ |
| I-176 | +++ |
| I-177 | +++ |
| I-178 | +++ |
| I-179 | +++ |
| I-180 | +++ |
| I-181 | +++ |
| I-182 | +++ |
| I-183 | +++ |
| I-184 | +++ |
| I-185 | +++ |
| I-186 | +++ |
| I-187 | +++ |
| I-188 | +++ |
| I-189 | +++ |
| I-190 | +++ |
| I-191 | +++ |
| I-192 | +++ |
| I-193 | +++ |
| I-194 | +++ |
| I-195 | +++ |
| I-196 | +++ |
| I-197 | +++ |
| I-198 | +++ |
| I-199 | +++ |
| I-200 | +++ |
| I-201 | +++ |
| I-202 | +++ |
| I-203 | +++ |
| I-204 | +++ |
| I-205 | +++ |
| I-206 | +++ |
| I-207 | +++ |
| I-208 | +++ |
| I-209 | +++ |
| I-210 | +++ |
| I-211 | +++ |
| I-212 | +++ |
| I-213 | +++ |
| I-214 | +++ |
| I-215 | +++ |
| I-216 | +++ |
| I-217 | +++ |
| I-218 | +++ |
| I-219 | +++ |
| I-220 | +++ |
| I-221 | +++ |
| I-222 | +++ |
| I-223 | +++ |
| I-224 | +++ |
| I-225 | +++ |
| I-226 | +++ |
| I-227 | +++ |
| I-228 | +++ |
| I-229 | +++ |
| I-230 | +++ |
| I-231 | +++ |
| I-232 | +++ |
| I-233 | +++ |
| I-234 | +++ |
| I-235 | +++ |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 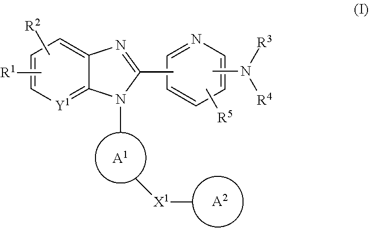
or a stereoisomer or pharmaceutically acceptable salt thereof; wherein:
R¹ is phenyl, a C₁₋₆ monocyclic cycloalkyl, a 5-6 membered heteroaryl containing 1 or 2 heteroatoms selected from nitrogen, a 5-6 membered saturated heterocyclyl containing 1 or 2 heteroatoms independently selected from nitrogen, oxygen, and sulfur, C₁₋₄ alkoxy or C₁₋₆ alkyl, wherein the phenyl, cycloalkyl, heteroaryl, heterocyclyl, and alkoxy are each substituted with m occurrences of R²;
R², R³, R⁴, R⁵, and R⁹ are each independently selected from hydrogen, C₁₋₄ alkyl, and C₁₋₄ haloalkyl;
R⁶ is halo or C₁₋₄ alkyl;
R⁷ is —(C₀₋₄ alkylene)-CO₂H, —CO₂(C₁₋₄ alkyl), S(O)₂N(R⁹)₂ or —C(O)N(R⁹)₂;
R⁸ represents independently for each occurrence C₁₋₄ alkyl, C₁₋₄ haloalkyl, or halo;
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R¹² represents independently for each occurrence halo, cyano, C₁₋₄ alkoxy or C₁₋₄ alkyl;
A¹ is phenylene or pyridinylene, each of which is substituted with 0, 1, or 2 occurrences of R⁶;
A² is a C₄₋₆ monocyclic cycloalkyl, C₅₋₈ bridged or fused bicyclic cycloalkyl, dioxo-cyclohexyl, C₇₋₁₀ spirocyclic saturated carbocyclyl, 7-10 membered spirocyclic saturated heterocyclyl containing 1 heteroatom selected from nitrogen or a 5-6 membered saturated heterocyclyl containing 1 or 2 heteroatoms independently selected from nitrogen, oxygen, and sulfur, wherein the cycloalkyl, carbocyclyl, and heterocyclyl are substituted with (i) 1 occurrence of R⁷ and (ii) n occurrences of R;
X¹ is —N(R⁹)C(O)-Ψ, —C(O)N(R⁹)-Ψ, -(3-6 membered saturated heterocyclylene containing 1 or 2 heteroatoms selected from nitrogen)-C(O)-Ψ, —N(R⁹)C(R¹⁰)(R¹¹)-Ψ, —N(R⁹)-Ψ, —C(O)-Ψ, —(C(R¹⁰)(R¹¹))ₚ-Ψ, -(3-6 membered saturated heterocyclylene containing 1 or 2 heteroatoms selected from nitrogen)-C(R¹⁰)(R¹¹)-Ψ, —(C(R¹⁰)(R¹¹))ₚ—N(R⁹)-Ψ, or a covalent bond; wherein Ψ is a bond to A², and wherein the 3-6 membered saturated heterocyclylene is substituted with 0, 1, or 2 occurrences of C₁₋₄ alkyl;
Y¹ is N or —C(H)—;
p is 1, 2, 3, 4, 5, or 6 and
m and n are independently 0, 1, 2, 3, or 4,
wherein when X¹ is a covalent bond, A² is not cyclobutyl.

2. The compound of claim 1, wherein the compound is represented by Formula I: 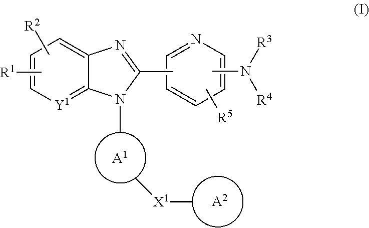
or a stereoisomer or pharmaceutically acceptable salt thereof; wherein:
R¹ is phenyl, a C₃₋₆ monocyclic cycloalkyl, a 5-6 membered heteroaryl containing 1 or 2 heteroatoms selected from nitrogen, a 5-6 membered saturated heterocyclyl containing 1 or 2 heteroatoms independently selected from nitrogen, oxygen, and sulfur, C₁₋₄ alkoxy or C₁₋₆ alkyl, wherein the phenyl, cycloalkyl, heteroaryl, heterocyclyl, and alkoxy are each substituted with m occurrences of R¹²;
R², R³, R⁴, R⁵, and R⁹ are independently hydrogen or C₁₋₄ alkyl;
R⁶ is halo or C₁₋₄ alkyl;
R⁷ is —(C₀₋₄ alkylene)-CO₂H, —CO₂(C₁₋₄ alkyl), or —C(O)NH₂;
R⁸ represents independently for each occurrence C₁₋₄ alkyl, C₁₋₄ haloalkyl, or halo;
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R² represents independently for each occurrence halo, cyano, or C₁₋₄ alkyl;
A¹ is phenylene or pyridinylene, each of which is substituted with 0, 1, or 2 occurrences of R⁶;
A² is a C₄₋₆ monocyclic cycloalkyl, C₅₋₇ bridged or fused bicyclic cycloalkyl, dioxo-cyclohexyl, C₇₋₁₀ spirocyclic saturated carbocyclyl, 7-10 membered spirocyclic saturated heterocyclyl containing 1 heteroatom selected from nitrogen or a 5-6 membered saturated heterocyclyl containing 1 or 2 heteroatoms independently selected from nitrogen, oxygen, and sulfur, wherein the cycloalkyl, carbocyclyl, and heterocyclyl are substituted with (i) 1 occurrence of R⁷ and (ii) n occurrences of R⁸;
X¹ is —N(R⁹)C(O)-Ψ, —C(O)N(R⁹)-Ψ, -(3-5 membered saturated heterocyclylene containing 1 or 2 heteroatoms selected from nitrogen)-C(O)-Ψ, —N(R⁹)C(R¹⁰)(R¹¹)-Ψ, —N(R⁹)-Ψ, —C(O)-Ψ, —C(R¹⁰)(R¹¹)-Ψ, -(3-6 membered saturated heterocyclylene containing 1 or 2 heteroatoms selected from nitrogen)-C(R¹⁰)(R¹¹)-Ψ, —C(R¹⁰)(R¹¹)—N(R⁹)-Ψ, or a covalent bond;
wherein Ψ is a bond to A²;
Y¹ is N or —C(H)—; and
m and n are independently 0, 1, 2, or 3,
wherein when X¹ is a covalent bond, A² is not cyclobutyl.

3. The compound of claim 1 or 2, wherein the compound is a compound of Formula I.

4. The compound of claim 1 or 2, wherein the compound is represented by Formula Ia or a stereoisomer or pharmaceutically acceptable salt thereof:

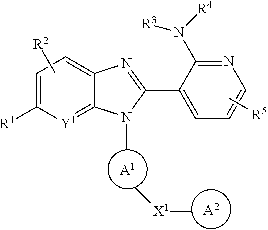

5. The compound of any one of claims 1-4, wherein Y¹ is N.

6. The compound of any one of claims 1-4, wherein Y¹ is —C(H)—.

7. The compound of any one of claims 1-6, wherein R¹ is phenyl.

8. The compound of any one of claims 1-6, wherein R¹ is C₁₋₆ alkyl.

9. The compound of any one of claims 1-6, wherein R¹ is a C₃₋₆ monocyclic cycloalkyl.

10. The compound of any one of claims 1-6, wherein R¹ is a 5-6 membered heteroaryl containing 1 or 2 heteroatoms selected from nitrogen.

11. The compound of any one of claims 1-6, wherein R¹ is a 5-6 membered saturated heterocyclyl containing 1 or 2 heteroatoms independently selected from nitrogen, oxygen, and sulfur.

12. The compound of any one of claims 1-6, wherein R¹ is a C₁₋₄ alkoxy.

13. The compound of any one of claims 1-12, wherein R² is hydrogen.

14. The compound of any one of claims 1-13, wherein R³ is hydrogen.

15. The compound of any one of claims 1-14, wherein R⁴ is hydrogen.

16. The compound of any one of claims 1-15, wherein R⁵ is hydrogen.

17. The compound of any one of claims 1-16, wherein X¹ is —N(R⁹)C(O)-Ψ.

18. The compound of any one of claims 1-16, wherein X¹ is —(C₁₋₃ alkylene)-N(R⁹)C(O)-Ψ.

19. The compound of any one of claims 1-16, wherein X¹ is —(CH₂)—N(R⁹)C(O)-Ψ.

20. The compound of any one of claims 1-16, wherein X¹ is a -(3-6 membered saturated heterocyclylene containing 1 or 2 heteroatoms selected from nitrogen)-C(O)-Ψ.

21. The compound of any one of claims 1-16, wherein X¹ is a -(4-membered saturated heterocyclylene containing 1 heteroatom selected from nitrogen)-C(O)-Ψ.

22. The compound of any one of claims 1-16, wherein X¹ is

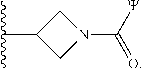

23. The compound of any one of claims 1-16, wherein X¹ is a -(3-6 membered saturated heterocyclylene containing 1 or 2 heteroatoms selected from nitrogen)-C(R¹⁰)(R¹¹)-Ψ.

24. The compound of any one of claims 1-19, wherein R⁹ is hydrogen.

25. The compound of any one of claims 1-19, wherein R⁹ is C₁₋₄ haloalkyl.

26. The compound of any one of claims 1-25, wherein A¹ is phenylene substituted with 0, 1, or 2 occurrences of R⁶.

27. The compound of any one of claims 1-25, wherein A¹ is phenylene substituted with 0 or 1 occurrence of R⁶.

28. The compound of any one of claims 1-25, wherein A¹ is phenylene.

29. The compound of any one of claims 1-25, wherein A¹ is pyridinylene.

30. The compound of any one of claims 1-29, wherein A² is cyclohexyl, cyclopentyl, or cyclobutyl, each of which is substituted with (i) 1 occurrence of R⁷ and (ii) n occurrences of R⁸.

31. The compound of any one of claims 1-29, wherein A² is cyclohexyl substituted with (i) 1 occurrence of R⁷ and (ii) n occurrences of R⁸.

32. The compound of any one of claims 1-29, wherein A² is cyclopentyl substituted with (i) 1 occurrence of R⁷ and (ii) n occurrences of R⁸.

33. The compound of any one of claims 1-29, wherein A² is C₅₋₈ bridged bicyclic cycloalkyl substituted with (i) 1 occurrence of R⁷ and (ii) n occurrences of R⁸.

34. The compound of any one of claims 1-29, wherein A² is a 5-6 membered saturated heterocyclyl containing 1 or 2 heteroatoms independently selected from nitrogen, oxygen, and sulfur, wherein the heterocyclyl is substituted with (i) 1 occurrence of R⁷ and (ii) n occurrences of R⁸.

35. The compound of any one of claims 1-29, wherein A² is piperidinyl or pyrrolidinyl, each of which is substituted with (i) 1 occurrence of R⁷ and (ii) n occurrences of R⁸.

36. The compound of any one of claims 1-29, wherein A² is a C₇₋₁₀ spirocyclic saturated carbocyclyl, wherein the carbocyclyl is substituted with (i) 1 occurrence of R⁷ and (ii) n occurrences of R⁸.

37. The compound of any one of claims 1-29, wherein A² is a 7-10 membered spirocyclic saturated heterocyclyl containing 1 heteroatom selected from nitrogen, wherein the heterocyclyl is substituted with (i) 1 occurrence of R⁷ and (ii) n occurrences of R⁸.

38. The compound of any one of claims 1-29, wherein A² is dioxo-cyclohexyl.

39. The compound of any one of claims 1-29, wherein A² is

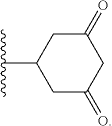 [SMILES: *C1CC(=O)CC(=O)C1]

40. The compound of any one of claims 1-29, wherein A² is one of the following

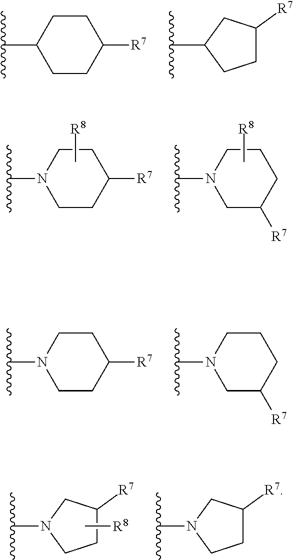

41. The compound of any one of claims 1-40, wherein n is 1.

42. The compound of any one of claims 1-40, wherein n is 0.

43. The compound of any one claims 1-40, wherein n is 4.

44. The compound of any one of claims 1-43, wherein m is 3.

45. The compound of any one of claims 1-43, wherein m is 2.

46. The compound of any one of claims 1-43, wherein m is 1.

47. The compound of any one of claims 1-43, wherein m is 0.

48. The compound of any one of claims 1-47, wherein p is 1.

49. The compound of any one of claims 1-47, wherein p is 2.

50. The compound of claim 1 or 2, wherein the compound is a compound of Formula Ib, Ic, Id, Ie, If, or Ig, or a stereoisomer or pharmaceutically acceptable salt thereof:

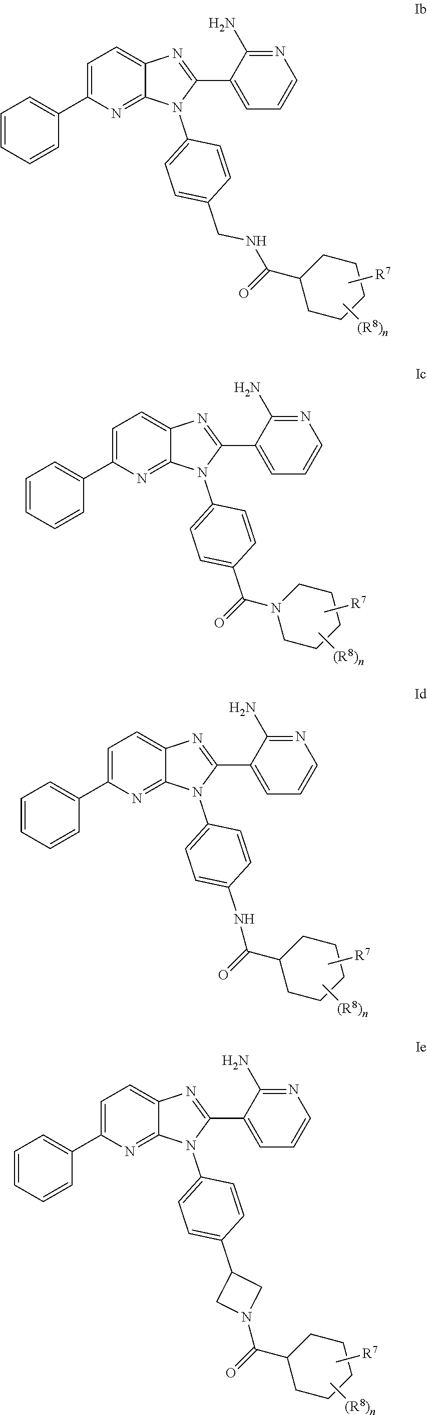

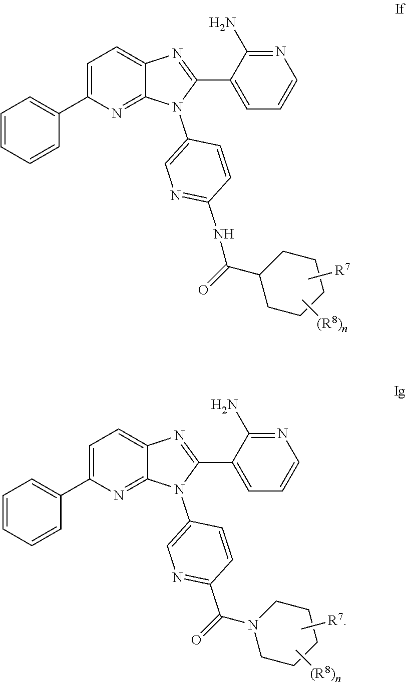

51. The compound of claim 1 or 2, wherein the compound is a compound of Formula Ih, Ii, Ij, Ik, Il, or Im, or a stereoisomer or pharmaceutically acceptable salt thereof:

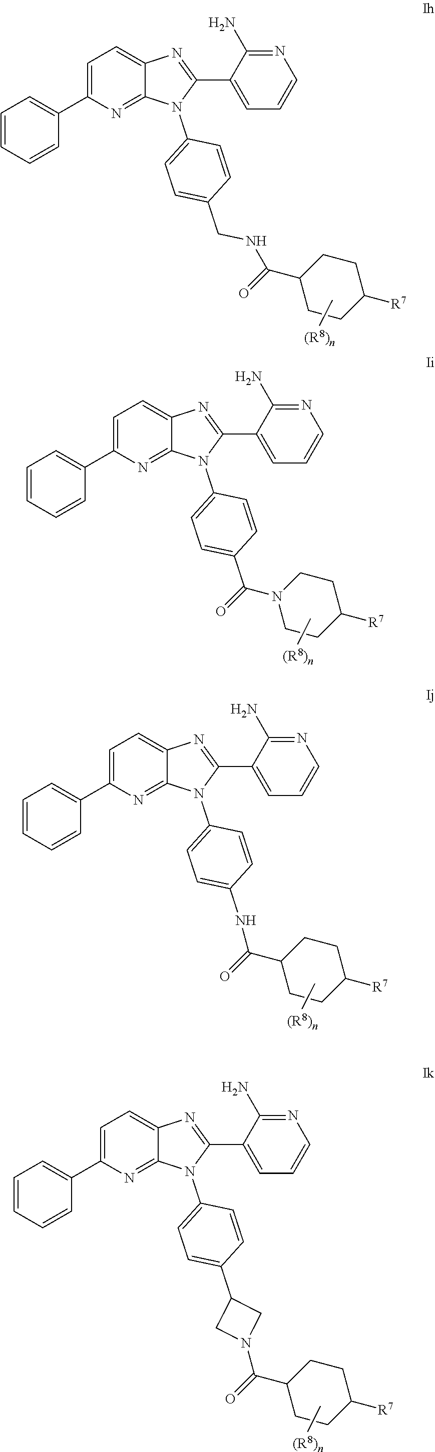

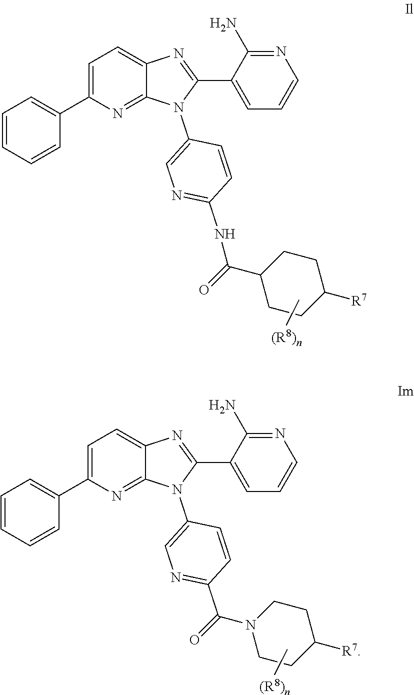

52. The compound of any one of claims 1-37 or 40-49, wherein R¹ represents independently for each occurrence C₁₋₄ alkyl.

53. The compound of any one of claims 1-37 or 40-49, wherein R¹ is methyl.

54. The compound of any one of claims 1-53, wherein R⁷ is —CO₂H.

55. The compound of any one of claims 1-53, wherein R⁷ is —(C₁₋₃ alkylene)-CO₂H.

56. The compound of any one of claims 1-53, wherein R⁷ is —CH₂CO₂H.

57. The compound of any one of claims 1-53, wherein R⁷ is —C(H)(CH₃)CO₂H.

58. The compound of any one of claims 1-53, wherein R⁷ is —CO₂CH₃.

59. The compound of any one of claims 1-53, wherein R⁷ is —C(O)NH₂.

60. The compound of any one of claims 1-53, wherein R⁷ is S(O)₂NHCH₃.

61. The compound of any one of claims 1-53, wherein R⁷ is C(O)NHCH₃.

62. A compound represented by Formula II: 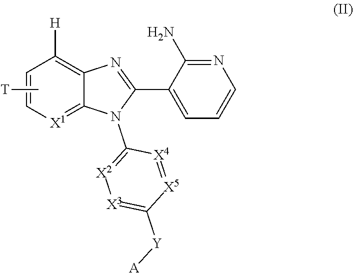
or a stereoisomer or pharmaceutically acceptable salt thereof, wherein:
T is hydrogen, halo, cyano, C(═O)NH₂, C₁₋₆alkyl, C₁₋₆ alkoxyl, C₃₋₂ cycloalkyl, 3-12 membered heterocyclyl, C₆₋₁₀ aryl, or 5-9 membered heteroaryl, wherein the C₁₋₆ alkyl, C₁₋₆ alkoxyl, C₆₋₁₀ aryl, or 5-9 membered heteroaryl are optionally substituted with one or more occurrences of R^{T};
R^{T} is independently for each occurrence halo, C₁₋₆ alkoxyl, C₁₋₆ alkyl, cyano, or NHC(═O)R^{T1};
R^{T1} is C₆₋₁₀ aryl or C₁₋₆ alkyl;
X¹ is N or CH;
X² and X³ are independently for each occurrence N or CR^{X};
X⁴ and X⁵ are CR^{X};
R^{X} is independently for each occurrence hydrogen, C₁₋₆ alkyl, or halo:
Y is absent, or Y is selected from the group consisting of —(C(R^{Y1})₂)_{q}—, —R^{Y2}C(R^{Y1})₂—*, —C(R^{Y1})₂R^{Y2}—*, —NR^{Y1}—, —(C(R^{Y1})₂)_{q}NR^{Y1}—*, —NR^{Y1}C(R^{Y1})₂—*, —CH₂NR^{Y1}(C═O)*, —(C═O)NR^{Y1}CH₂—*, —NR^{Y1}C(═O)—*, —C(═O)NR^{Y1}—*, —C(═O)—, —R^{Y1}C(═O)—*, and —C(═O)R^{Y2}—*, wherein * denotes an attachment to A:
R^{Y1} is independently for each occurrence hydrogen, C₁₋₆ alkyl, or C₁₋₆ haloalkyl;
R^{Y2} is 3-12 membered heterocyclyl, wherein the 3-12 membered heterocyclyl is optionally substituted with one or more occurrences of C₁₋₆ alkyl;
q is 1, 2, 3, 4,5 or 6;
A is C₃₋₁₂ cycloalkyl or 3-12 membered heterocyclyl, wherein the C₃₋₁₂ cycloalkyl or 3-12 membered heterocyclyl are substituted with one or more occurrences of R^{A};
R^{A} is independently for each occurrence oxo, halo, C₁₋₆ alkyl, —(C₀₋₆ alkylene)-C(═O)OR^{A1}, C(═O)N(R^{A1})₂, NHC(═O)R^{A1}, NHC(═O)OR^{A1}, OC(═O)N(R^{A1})₂, S(═O)₂OR^{A1}, or S(═O)₂N(R^{A1})₂; and
R^{A1} is independently for each occurrence hydrogen or C₁₋₆ alkyl,
wherein when Y is absent, A is not cyclobutyl, azetidinyl, or oxetanyl.

63. The compound of claim 62, wherein the compound is represented by Formula II: 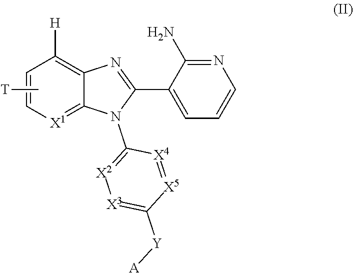
or a stereoisomer or pharmaceutically acceptable salt thereof, wherein:
T is hydrogen, halo, cyano, C(═O)NH₂, C₁₋₆ alkyl, C₁₋₆ alkoxyl, C₃₋₁₂ cycloalkyl, 3-12 membered heterocyclyl, C₆₋₁₀ aryl, or 5-9 membered heteroaryl, wherein the C₁₋₆ alkyl, C₁₋₆ alkoxyl, C₆₋₁₀ aryl, or 5-9 membered heteroaryl are optionally substituted with one or more occurrences of R^{T};
R^{T} is independently for each occurrence halo, C₁₋₆ alkyl, cyano, or NHC(═O)R^{T1};
R^{T1} is C₆₋₁₀ aryl or C₁₋₆ alkyl;
X¹ is N or CH;
X² and X³ are independently for each occurrence N or CR^{X};
X⁴ and X⁵ are CR^{X};
R^{X} is independently for each occurrence hydrogen, C₁₋₆ alkyl, or halo;
Y is absent, or Y is selected from the group consisting of —C(R^{Y1})₂—, —R^{Y2}C(R^{Y1})₂—*, —C(R^{Y1})₂R^{Y2}—*, —NR^{Y1}—, —C(R^{Y1})₂NR^{Y1}—*, —NR^{Y1}C(R^{Y1})₂—*, —CH₂NR^{Y1}(C═O)—*, —(C═O)NR^{Y1}CH₂—*, —NR^{Y1}C(═O)—*, —C(═O)NR^{Y1}—*, —C(═O)—, —R^{Y2}C(═O)—*, and —C(═O)R^{Y2}—*, wherein * denotes an attachment to A;
R^{Y1} is independently for each occurrence hydrogen or C₁₋₆ alkyl;
R^{Y2} is 3-12 membered heterocyclyl;
A is C₃₋₁₂cycloalkyl or 3-12 membered heterocyclyl, wherein the C₃₋₁₂ cycloalkyl or 3-12 membered heterocyclyl are substituted with one or more occurrences of R^{A};
R^{A} is independently for each occurrence oxo, halo, C₁₋₆ alkyl, —(C₀₋₆ alkylene)-C(═O)OR^{A1}, C(═O)N(R^{A1})₂, NHC(═O)R^{A1}, NHC(═O)OR^{A1}, OC(═O)N(R^{A1})₂, S(═O)₂OR^{A1}, or S(═O)₂N(R^{A1})₂; and
R^{A1} is independently for each occurrence hydrogen or C₁₋₆ alkyl,
wherein when Y is absent, A is not cyclobutyl, azetidinyl, or oxetanyl.

64. The compound of claim 62 or 63, wherein X¹ is N.

65. The compound of claim 62 or 63, wherein X¹ is CH.

66. The compound of any one of claims 62-65, wherein T is phenyl, C₁₋₄ alkyl, C₅₋₆ cycloalkyl, C₁₋₃ alkoxy, 5-6 membered heteroaryl, or 6-membered heterocyclyl, wherein the phenyl, C₁₋₃ alkoxy, or 5-6 membered heteroaryl are optionally substituted with one or more occurrences of R^{T}.

67. The compound of any one of claims 62-66, wherein T is phenyl, methyl, tert-butyl, cyclopentyl, cyclohexyl, methoxy, propoxy, morpholinyl, tetrahydropyranyl, pyrazolyl, or pyridinyl, wherein the phenyl, methoxy, or pyrazolyl are optionally substituted with one or more occurrences of R^{T}.

68. The compound of any one of claims 62-67, wherein T is phenyl optionally substituted with one or more occurrences of R^{T}.

69. The compound of any one of claims 62-68, wherein R^{T} is halo, C₁₋₆ alkoxyl, C₁₋₆ alkyl, or cyano.

70. The compound of any one of claims 62-69, wherein R^{T} is fluoro, methoxy, methyl, or cyano.

71. The compound of any one of claims 62-70, wherein X² is N and X³ is CR^{X}.

72. The compound of any one of claims 62-70, wherein X² is CR^{X} and X³ is N.

73. The compound of any one of claims 62-70, wherein X² is CR^{X} and X³ is CR^{X}.

74. The compound of any one of claims 62-73, wherein R^{X} is hydrogen, methyl, or fluoro.

75. The compound of any one of claims 62-74, wherein R^{Y1} is hydrogen, methyl, or iso-butyl.

76. The compound of any one of claims 62-75, wherein R^{Y2} is azetidinyl, piperidinyl, or pyrrolidinyl.

77. The compound of any one of claims 62-76, wherein Y is —NHC(═O)—*, —NHCH₂—*, —NH—, —C(═O)NH—*, —CH₂—, —CH₂CH₂—, —CH₂NH—*, —CH₂CH₂N(CH₃)—*, azetidinyl-CH₂—*, azetidinyl-(C═O)—*, —CH₂NH(C═O)—*, azetidinyl-CH(CH₃)—*, azetidinyl-CH(CH(CH₃)₂)—*, piperidinyl-CH₂—*, pyrrolidinyl-CH₂—*, —CH(CH₃)—*, —CH(CH₃)NH—*, —C(CH₃)₂NH—*, —CH₂N(CH₃)—, or —C(═O)—.

78. The compound of any one of claims 62-77, wherein q is 1 or 2.

79. The compound of any one of claims 62-78, wherein A is C₅₋₇ cycloalkyl or 5-7 membered heterocyclyl, wherein the C₅₋₇ cycloalkyl or 5-7 membered heterocyclyl are substituted with one or more occurrences of R^{A}.

80. The compound of any one of claims 62-79, wherein A is cyclopentyl, bicyclo[1.1.1]pentyl, cyclohexyl, bicyclo[2.1.1]hexyl, bicyclo[3.1.0]hexyl, spiro[3.3]heptyl, pyrrolidinyl, piperidinyl, or 2-azaspiro[3 3]heptyl, wherein the cyclopentyl, bicyclo[1.1.1]pentyl, bicyclo[2.1.1]hexyl, bicyclo[3.1.0]hexyl, spiro[3.3]heptyl, pyrrolidinyl, piperidinyl, or 2-azaspiro[3.3]heptyl are substituted with one or more occurrences of R^{A}.

81. The compound of any one of claims 62-76, wherein Y is absent.

82. The compound of any one of claims 62-78 and 81, wherein A is pyrrolidinyl, piperidinyl, or 2-azaspiro[3.3]heptyl, wherein the pyrrolidinyl, piperidinyl, or 2-azaspiro[3.3]heptyl are substituted with one or more occurrences of R^{A}.

83. The compound of any one of claims 62-82, wherein R^{A} is oxo, halo, C₁₋₆ alkyl, —(C₀₋₃ alkylene)-C(═O)OR^{A1}, or C(═O)N(R^{A1})₂.

84. The compound of any one of claims 62-83, wherein R^{A} is oxo, fluoro, methyl, —C(═O)OH, —CH(CH₃)C(═O)OH, —CH₂C(═O)OH, —C(CH₃)₂C(═O)OH, —C(═O)OCH₃, or C(═O)NH₂.

85. The compound of any one of claims 62-84, wherein R^{A1} is hydrogen or methyl.

86. The compound of any one of claims 62-85, wherein R^{A} is —C(═O)OH.

87. The compound of claim 62 or 63, wherein the compound is a compound of Formula IIa, IIb, IIc, or IId or a stereoisomer or pharmaceutically acceptable salt thereof:

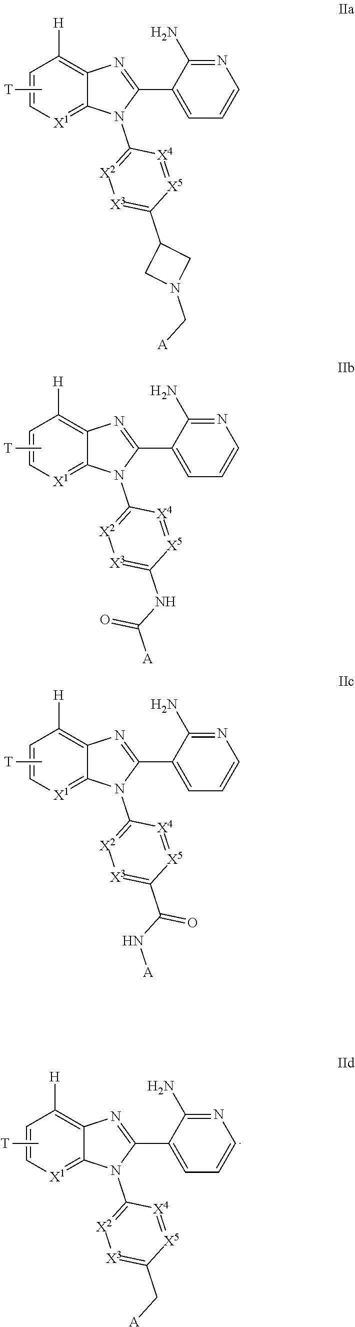

88. A compound in Table 1 herein, or a stereoisomer or pharmaceutically acceptable salt thereof.

89. A pharmaceutical composition comprising a compound of any one of claims 1-88 and a pharmaceutically acceptable carrier.

90. A method of treating a disease or disorder associated with aberrant AKT1 signaling, comprising administering to a subject in need thereof a therapeutically effective amount of a compound of any one of claims 1-88 to treat the disease or disorder.

91. The method of claim 90, wherein the disease or disorder associated with aberrant AKT1 signaling is a disease or disorder associated with an AKT E17K genetic mutation.

92. A method of treating cancer, comprising administering to a subject in need thereof a therapeutically effective amount of a compound of any one of claims 1-88 to treat the cancer.

93. The method of claim 92, wherein the cancer is a solid tumor.

94. The method of claim 92, wherein the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, leukemia, urothelial cancer, colorectal cancer, or glioblastoma multiforme.

95. The method of claim 92, wherein the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, uterine corpus endometrial carcinoma, cervical cancer, endocervical cancer, thyroid carcinoma, prostate adenocarcinoma, skin cutaneous melanoma, bladder urothelial carcinoma, head and neck squamous cell carcinoma, or stomach adenocarcinoma.

96. The method of claim 92, wherein the cancer is an adenocarcinoma, squamous cell carcinoma, epithelial neoplasm, glioma, ductal neoplasm, lobular neoplasm, cystic neoplasm, mucinous neoplasm, or serous neoplasm, acinar cell neoplasm, basal cell neoplasm, fibroepithelial neoplasm, transitional cell papilloma, or transitional cell carcinoma.

97. The method of claim 92, wherein the cancer is a cervical cancer, uterine cancer, breast cancer, thyroid cancer, prostate cancer, lung cancer, bladder cancer, skin cancer, stomach cancer, lymphoma, or leukemia.

98. The method of claim 92, wherein the cancer is a lymphoma or leukemia.

99. The method of any one of claims 92-98, wherein the cancer has an AKT1 mutation.

100. The method of any one of claims 92-98, wherein the cancer has an AKT1 E17K mutation.

101. A method of treating a disease or disorder associated with active PI3K signaling, comprising administering to a subject in need thereof a therapeutically effective amount of a compound of any one of claims 1-88 to treat the disease or disorder.

102. The method of any one of claims 90-101, wherein the subject is a human.

103. A method of inhibiting AKT1 activity, comprising contacting an AKT1 with an effective amount of a compound of any one of claims 1-88 to thereby inhibit the AKT1 activity.

104. The method of claim 103, wherein the AKT1 is an AKT1 E17K.
